(19) United States
(12) Patent Application Publication
(10) Pub. No.: US 2024/0383874 A1
(43) Pub. Date: Nov. 21, 2024

(54) BENZENE RING DERIVATIVE, AND COMPOSITION AND PHARMACEUTICAL USE THEREOF

(71) Applicant: Xizang Haisco Pharmaceutical Co., Ltd., Lhoka (CN)

(72) Inventors: Chen Zhang, Chengdu (CN); Yuting Liao, Chengdu (CN); Fei Ye, Chengdu (CN); Pingming Tang, Chengdu (CN); Xiaogang Chen, Chengdu (CN); Yonghua Lu, Chengdu (CN); Qiu Gao, Chengdu (CN); Yao Li, Chengdu (CN); Jia Ni, Chengdu (CN); Pangke Yan, Chengdu (CN)

(21) Appl. No.: 18/254,374

(22) PCT Filed: Nov. 24, 2021
(86) PCT No.: PCT/CN2021/132803

§ 371 Date: May 24, 2023

(30) Foreign Application Priority Data
Nov. 25, 2020 (CN) 202011329463.4
Jan. 29, 2021 (CN) 202110123667.0
Apr. 16, 2021 (CN) 202110403688.8
Nov. 1, 2021 (CN) 202110006143.3

(51) Int. Cl. C07D 401/14; A61K 31/444; A61K 31/506; C07D 417/14; C07D 487/04; C07D 487/10

(52) U.S. Cl. CPC C07D 401/14; A61K 31/444; A61K 31/506; C07D 417/14; C07D 487/04; C07D 487/10

(57) ABSTRACT

Disclosed are a compound as represented by general formula (I) or a stereoisomer, deuterated compound, solvate, prodrug, metabolite, pharmaceutically acceptable salt or co-crystal thereof, and an intermediate thereof, and the use thereof in AR or AR splicing mutant-related diseases such as cancer.

B-L-K (I)

The present application is based on and claims the right of priority for the application with the CN application no. being 202011329463.4 and the filing date being Nov. 25, 2020; the application with the CN application no. being 202110006143.3 and the filing date being Jan. 11, 2021; the application with the CN application no. being 202110123667.0 and the filing date being Jan. 29, 2021; and the application with the CN application no. being 202110403688.8 and the filing date being Apr. 16, 2021, and the disclosure of these CN applications is incorporated herein by reference in its entirety.

### TECHNICAL FIELD

The present invention relates to a compound as represented by general formula (I) or a stereoisomer, deuterated compound, solvate, prodrug, metabolite, pharmaceutically acceptable salt or co-crystal thereof, and an intermediate thereof and a preparation method therefor, and the use thereof in AR or AR splice variant-related diseases such as cancer or an autoimmune system disease.

### BACKGROUND ART

The androgen receptor (AR), a nuclear hormone receptor that is structurally comprised of an N-terminal domain (NTD), a DNA-binding domain (DBD) and a ligand-binding domain (LTD), can regulate the expression of genes that induce prostate cancer, and thus, inhibition of the androgen receptor is an effective method for treating prostate cancer. Currently marketed androgen receptor inhibitors such as enzalutamide and bicalutamide mainly exert an inhibitory effect by interacting with the ligand-binding domain (LTD) of the androgen receptor. However, some patients will develop drug resistance caused by androgen receptor splice variants (AR-Vs) with the deletion of LTD fragment during treatment. Preclinical studies have shown that androgen receptor splice variants can accelerate the progression of enzalutamide-resistant prostate cancer, and therefore, how to solve the problem of drug resistance has become the focus of clinical medicine.

PROTAC (proteolysis targeting chimera) molecules are a class of dual function compounds which are capable of binding to both targeted proteins and E3 ubiquitin ligases. This class of compounds can be recognized by proteasomes in a cell to cause the degradation of the targeted proteins, which can effectively reduce the contents of the targeted proteins in the cell. By introducing a ligand capable of binding to various targeted proteins into the PROTAC molecules, it is possible to apply the PROTAC technology to the treatment of various diseases, and this technology has attracted extensive attention in recent years.

Therefore, it is necessary to develop a novel androgen receptor (AR) or AR splice variant (AR-V) inhibitor and a PROTAC drug of E3 ubiquitin ligase for the treatment of AR or AR splice variant-related tumor diseases.

### SUMMARY OF THE INVENTION

An objective of the present invention is to provide a compound with a novel structure, good efficacy, high bioavailability and higher safety that can inhibit and degrade androgen receptor splice variants, for use in the treatment of AR or AR splice variant-related diseases such as an autoimmune disease, an inflammatory disease or cancer.

The compound of the present invention has good activity of inhibiting and/or degrading AR or AR splice variants, good pharmacokinetic performance and bioavailability, oral performance and good safety.

The present invention provides a compound or a stereoisomer, deuterated compound, solvate, prodrug, metabolite, pharmaceutically acceptable salt or co-crystal thereof, wherein the compound is selected from a compound as represented by general formula (I),

B-L-K (I);
in some embodiments, L is selected from a bond or C₁₋₅₀ hydrocarbyl, wherein 0 to 20 methylene units in the hydrocarbyl are replaced by -Ak- and -Cy-;
in some embodiments, each -Ak- is independently selected from Ak1, Ak2, Ak3, Ak4 or Ak5;
in some embodiments, each -Ak- is independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}, —(C≡C)_{q}—, —CH═CH—, —Si(R^{L})₂—, —Si(OH)(R^{L})—, —Si(OH)₂—, —P(═O)(OR^{L})—, —P(═O)(R^{L}), —S—, —S(═O), —S(═O)₂— or a bond;
in some embodiments, each -Cy- is independently selected from Cy1, Cy2, Cy3 or Cy4;
in some embodiments, each -Cy- is independently selected from a bond, 4- to 8-membered mono-heterocyclic ring, 4- to 10-membered fused heterocyclic ring, 5- to 12-membered spiro-heterocyclic ring, 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 heteroatoms selected from O, S or N;
in some embodiments, L is selected from -Cy1-Cy2-Cy3-Cy4-Ak1-Ak2-Ak3-Ak4-Ak5-, -Cy1-Ak1-Cy2-Ak2-Cy3-Ak3-Cy4-Ak4-Ak5-, -Ak1-Cy1-Ak2-Cy2-Ak3-Cy3-Ak4-Cy4-Ak5-, -Cy1-Ak1-Cy2-Ak2-Cy3-Cy4-Ak3-Ak4-Ak5-, -Cy1-Ak1-Cy2-Ak2-Ak3-Cy3-Cy4-Ak4-Ak5-, -Cy1-Ak1-Ak2-Ak3-Ak4-Ak5-Cy2-Cy3-Cy4-, -Cy1-Cy2-Ak1-Ak2-Ak3-Ak4-Ak5-Cy3-Cy4-, -Cy1-Cy2-Cy3-Ak1-Ak2-Ak3-Ak4-Ak5-Cy4-, -Cy1-Cy2-Cy3-Cy4-Ak1-Ak2-Ak3-Ak4-Ak5-, -Cy1-Ak1-Cy2-Cy3-Cy4-Ak2-Ak3-Ak4-Ak5-, -Cy1-Cy2-Ak1-Cy3-Cy4-Ak2-Ak3-Ak4-Ak5-, -Cy1-Cy2-Cy3-Ak1-Cy4-Ak2-Ak3-Ak4-Ak5-, -Cy1-Ak1-Ak2-Cy2-Cy3-Cy4-Ak3-Ak4-Ak5-, -Cy1-Cy2-Ak1-Ak2-Cy3-Cy4-Ak3-Ak4-Ak5-, -Cy1-Cy2-Cy3-Ak1-Ak2-Cy4-Ak3-Ak4-Ak5-, -Cy1-Ak1-Ak2-Ak3-Cy2-Cy3-Cy4-Ak4-Ak5-, -Cy1-Cy2-Ak1-Ak2-Ak3-Cy3-Cy4-Ak4-Ak5-, -Cy1-Cy2-Cy3-Ak1-Ak2-Ak3-Cy4-Ak4-Ak5-, -Cy1-Ak1-Ak2-Ak3-Ak4-Cy2-Cy3-Cy4-Ak5-, -Cy1-Cy2-Ak1-Ak2-Ak3-Ak4-Cy3-Cy4-Ak5-, -Cy1-Cy2-Cy3-Ak1-Ak2-Ak3-Ak4-Cy4-Ak5-, -Ak1-Ak2-Ak3-Ak4-Ak5-Cy1-Cy2-Cy3-Cy4-, -Ak1-Cy1-Cy2-Cy3-Cy4-Ak2-Ak3-Ak4-Ak5-, -Ak1-Ak2-Cy1-Cy2-Cy3-Cy4-Ak3-Ak4-Ak5-, -Ak1-Ak2-Ak3-Cy1-Cy2-Cy3-Cy4-Ak4-Ak5-, -Ak1-Ak2-Ak3-Ak4-Cy1-Cy2-Cy3-Cy4-Ak5-, -Ak1-Cy1-Ak2-Ak3-Ak4-Ak5-Cy2-Cy3-Cy4-, -Ak1-Cy1-Cy2-Ak2-Ak3-Ak4-Ak5-Cy3-Cy4-, -Ak1-Cy1-Cy2-Cy3-Ak2-Ak3-Ak4-Ak5-Cy4-, -Ak1-Ak2-Cy1-Ak3-Ak4-Ak5-Cy2-Cy3-Cy4-, -Ak1-Ak2-Cy1-Cy2-Ak3-Ak4-Ak5-Cy3-Cy4-, -Ak1-Ak2-Cy1-Cy2-Cy3-Ak3-Ak4-Ak5-Cy4-, -Ak1-Ak2-Ak3-Cy1-Ak4-Ak5-Cy2-Cy3-Cy4-, -Ak1-Ak2-Ak3-Cy1-Cy2-Ak4-Ak5-Cy3-Cy4-, -Ak1-Ak2-Ak3-Cy1-Cy2-Cy3-Ak4-Ak5-Cy4-, -Ak1-Ak2-Ak3-Ak4-Cy1-Ak5-Cy2-Cy3-Cy4-, -Ak1-Ak2-Ak3-Ak4-Cy1-Cy2-Ak5-Cy3-Cy4-, or -Ak1-Ak2-Ak3-Ak4-Cy1-Cy2-Cy3-Ak5-Cy4-;
in some embodiments, Ak1, Ak2, Ak3, Ak4, and Ak5 are each independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, C(O)—, —C(═O)—(CH₂)_{q}—NR^{L}—, (C≡C)_{q}— or a bond;
in some embodiments, L is selected from -Cy1-, -Cy1-Ak1-, -Cy1-Ak1-Ak2-, -Cy1-Ak1-Ak2-Ak3-, -Cy1-Ak1-Ak2-Ak3-Ak4-, -Cy1-Cy2-, -Cy1-Ak1-Cy2-, -Cy1-Cy2-Ak2-, -Cy1-Ak1-Cy2-Ak2-, -Cy1-Ak1-Cy2-Ak2-Ak3-, -Cy1-Ak1-Cy2-Ak2-Ak3-Ak4-, -Cy1-Cy2-Ak2-Ak3-, -Cy1-Cy2-Ak2-Ak3-Ak4-, -Cy1-Ak1-Cy2-Ak2-Ak3-Ak4-, -Cy1-Ak1-Ak2-Cy3-, -Cy1-Ak1-Ak2-Cy3-Ak3-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Ak1-Cy2-Cy3-Ak3-, -Cy1-Cy2-Ak2-Cy3-Ak3-, -Cy1-Ak1-Cy2-Ak2-Cy3-, -Cy1-Ak1-Cy2-Ak2-Cy3-Ak3-, -Cy1-Cy2-Cy3-Ak3-Ak4-, -Cy1-Cy2-Cy3-Cy4-, -Cy1-Ak1-Cy2-Cy3-Cy4-, -Cy1-Cy2-Ak2-Cy3-Cy4-, -Cy1-Cy2-Cy3-Ak3-Cy4-, -Cy1-Cy2-Cy3-Cy4-Ak4-, -Cy1-Ak1-Cy2-Ak2-Cy3-Ak3-Cy4-, -Cy1-Ak1-Cy2-Ak2-Cy3-Cy4-, -Ak1-Ak2-Cy3-Cy4-, -Ak1-Cy2-Ak2-Cy3-, -Ak1-Cy2-Ak2-, -Ak1-Ak2-Ak3-Ak4-, -Ak1-Ak2-Ak3-, -Ak1-Ak2-, -Ak1-Ak2-Ak3-Ak4-Ak5-, -Cy1-Cy2-Cy3-Ak3-Ak4-Ak5-, -Cy1-Cy2-Ak2-Cy3-Ak3-Ak4-Ak5-, -Cy1-Ak1-Cy2-Ak2-Ak3-Ak4-Ak5-, -Cy1-Cy2-Cy3-Cy4-Ak4-Ak5-, -Cy1-Ak1-Ak2-Ak3-Ak4-Ak5-, -Ak1-Cy2-Ak2-Ak3-Ak4-Ak5-, -Ak1-Cy2-Ak2-Ak3-Ak4-, or -Ak1-Cy2-Ak2-Ak3-;
in some embodiments, Ak1, Ak2, Ak3, Ak4, and Ak5 are each independently selected from —O—, —OCH₂—, —CH₂O—, —OCH₂CH₂—, —CH₂CH₂O—, —C≡C—, —CH₂—, —CH₂CH₂—, —CH₂CH₂CH₂—, —N(CH₃)—, —NH—, —CH₂N(CH₃)—, —CH₂NH—, —NHCH₂—, —CH₂CH₂N(CH₃)—, —CH₂CH₂NH—, —NHCH₂CH₂—, —C(═O)—, —C(═O)CH₂NH—, —CH₂C(═O)NH—, —C(═O)NH— or —NHC(═O)—;
in some embodiments, L is selected from -Cy1-Ak1-Cy2-Ak2-Cy3-Ak3-Cy4-Ak4-; in some embodiments, Ak1, Ak2, Ak3 and Ak4 are each independently selected from —(CH₂)_{q}—, 0, —(CH₂)_{q}—NR^{L}, NR^{L}C(═O), C(═O), C(═O)—(CH₂)_{q}—NR^{L}, C≡C or a bond;
in some embodiments, L is selected from -Cy1-, -Cy1-Ak1-, -Cy1-Cy2-, -Cy1-Ak1-Cy2-, -Cy1-Cy2-Ak2-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Cy2-Ak2-Cy3-Ak3- and -Cy1-Ak1-Cy2-Ak2-Cy3-; Ak1, Ak2 and Ak3 are each independently selected from —C≡C—, —CH₂— and —CH₂—N(CH₃)—; Cy1, Cy2 and Cy3 are each independently selected from 4- to 7-membered mono-heterocyclic ring, 4- to 10-membered fused heterocyclic ring, 5- to 12-membered spiro-heterocyclic ring, wherein the mono-heterocyclic ring, fused heterocyclic ring or spiro-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring or spiro-heterocyclic ring contains 1 to 4 heteroatoms selected from O, S, or N (preferably, N);
in some embodiments, L is selected from -Cy1-, -Cy1-Ak1-, -Cy1-Cy2-, -Cy1-Ak1-Cy2-, -Cy1-Cy2-Ak2-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Cy2-Ak2-Cy3-Ak3- and -Cy1-Ak1-Cy2-Ak2-Cy3-; Ak1, Ak2 and Ak3 are each independently selected from —C≡C—, —CH₂— and —CH₂—N(CH₃)—; Cy1, Cy2 and Cy3 are each independently selected from

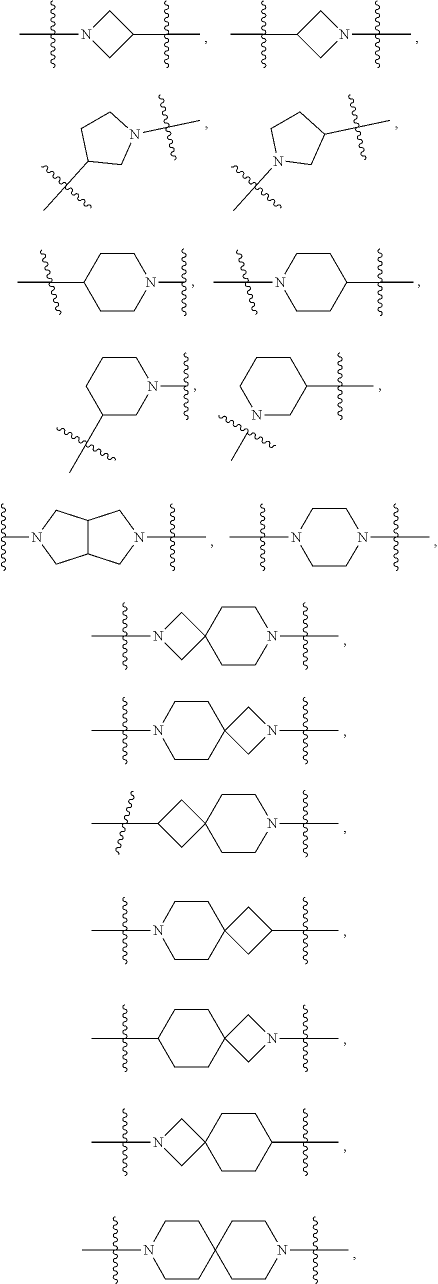

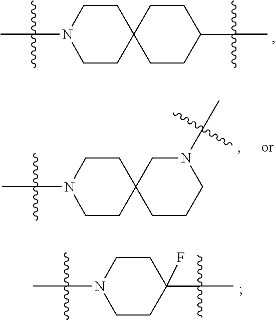

in some embodiments, each R^{L} is independently selected from H, C₁₋₆ alkyl, 3- to 7-membered heterocyclyl, 3- to 7-membered cycloalkyl, phenyl or 5- to 6-membered heteroaryl;
in some embodiments, each R^{L} is independently selected from H or C₁₋₆ alkyl;
in some embodiments, each R^{L} is independently selected from H or C₁₋₄ alkyl;
in some embodiments, each R^{L} is independently selected from H, methyl or ethyl;
in some embodiments, Cy1, Cy2, Cy3 and Cy4 are each independently selected from a bond, 4- to 7-membered mono-heterocyclic ring, 4- to 10-membered fused heterocyclic ring, 5- to 12-membered spiro-heterocyclic ring, 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 (for example, 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 (for example, 1, 2, 3 or 4) heteroatoms selected from O, S, or N;
in some embodiments, Cy1, Cy2, Cy3 and Cy4 are each independently selected from a bond, 4- to 7-membered (for example, 4-, 5-, 6- or 7-membered) nitrogen-containing mono-heterocyclic ring, 4- to 10-membered (for example, 4-, 5-, 6-, 7-, 8-, 9- or 10-membered) nitrogen-containing fused heterocyclic ring, 5- to 12-membered (for example, 5-, 6-, 7-, 8-, 9-, 10-, 11- or 12-membered) nitrogen-containing spiro-heterocyclic ring, 7- to 10-membered (for example, 7-, 8-, 9- or 10-membered) nitrogen-containing bridged-heterocyclic ring, 3- to 7-membered (for example, 3-, 4-, 5-, 6- or 7-membered) monocycloalkyl, 4- to 10-membered (for example, 4-, 5-, 6-, 7-, 8-, 9- or 10-membered) fused cycloalkyl, 5- to 12-membered (for example, 5-, 6-, 7-, 8-, 9-, 10-, 11- or 12-membered) spiro cycloalkyl, 7- to 10-membered (for example, 7-, 8-, 9- or 10-membered) bridged cycloalkyl, 5- to 10-membered (for example, 5-, 6-, 7-, 8-, 9- or 10-membered) heteroaryl or 6- to 10-membered (for example, 6-, 7-, 8-, 9- or 10-membered) aryl, wherein the mono-heterocyclic ring, fused heterocyclic ring, bridged-heterocyclic ring, spiro-heterocyclic ring, cycloalkyl, aryl or heteroaryl is optionally further substituted with 0 to 4 (for example, 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring, spiro-heterocyclic ring or heteroaryl contains 1 to 4 (for example, 1, 2, 3 or 4) heteroatoms selected from O, S, or N;
in some embodiments, Cy1, Cy2, Cy3 and Cy4 are each independently selected from a bond or one of the following substituted or unsubstituted groups: cyclopropyl, cyclobutyl, cyclopentyl, cyclohexyl, azetidinyl, azacyclopentyl, azacyclohexenyl, piperidine, morpholine, piperazine, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl-spiro-piperidine, azetidinyl-spiro-piperidine, azacyclopentyl-spiro-piperidine, azacyclohexyl-spiro-piperidine,

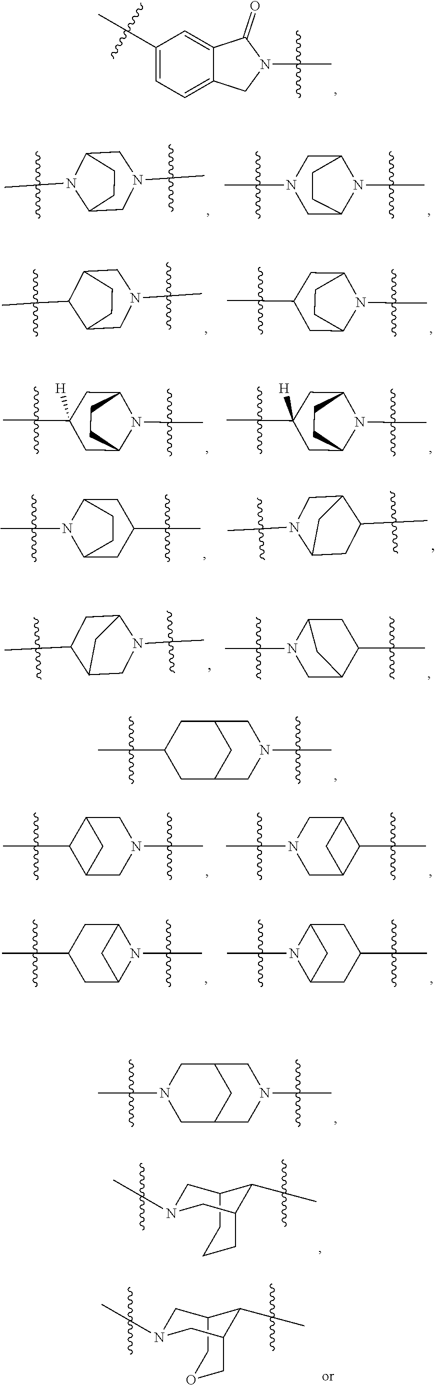

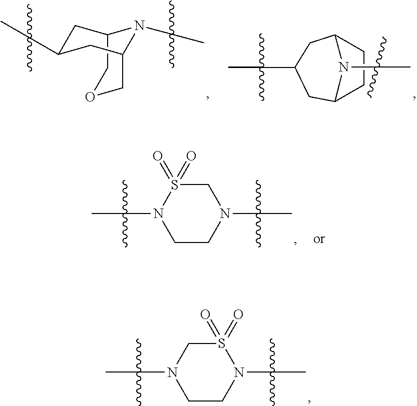 [SMILES: *C1CC2COCC(C1)N2* ; *C1CC2CCC(C1)N2* ; *N1CCN(*)S(=O)(=O)C1 ; *N1CCN(*)S(=O)(=O)C1]

which, when substituted, is optionally further substituted with 0 to 4 (for example, 0, 1, 2, 3 or 4) substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl or C₁₋₄ alkoxy;
in some embodiments, Cy1, Cy2, Cy3 and Cy4 are each independently selected from a bond or one of the following substituted or unsubstituted groups:

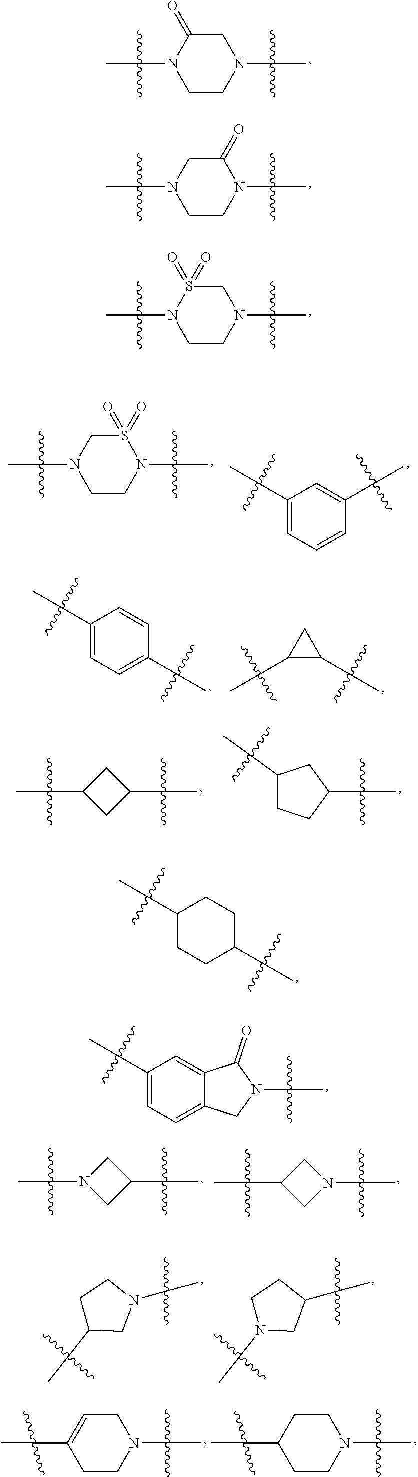

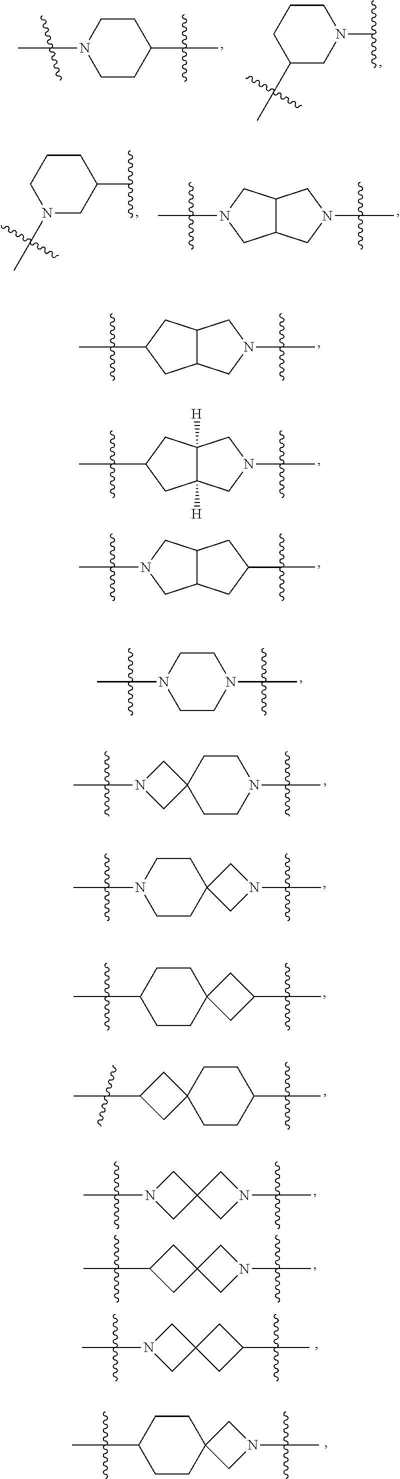

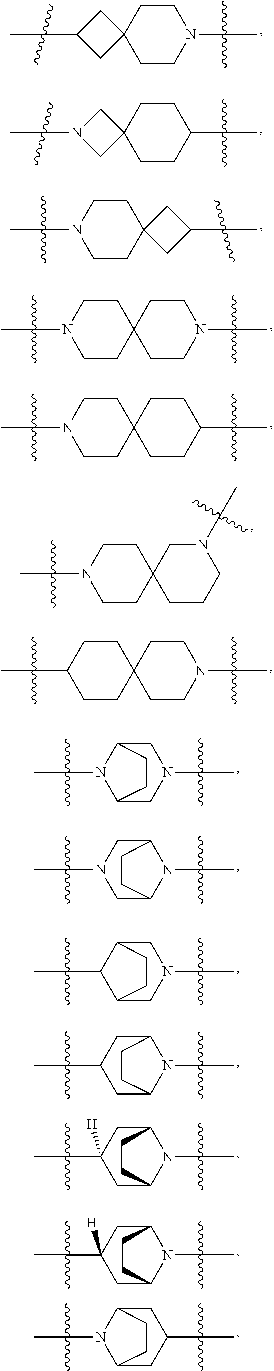

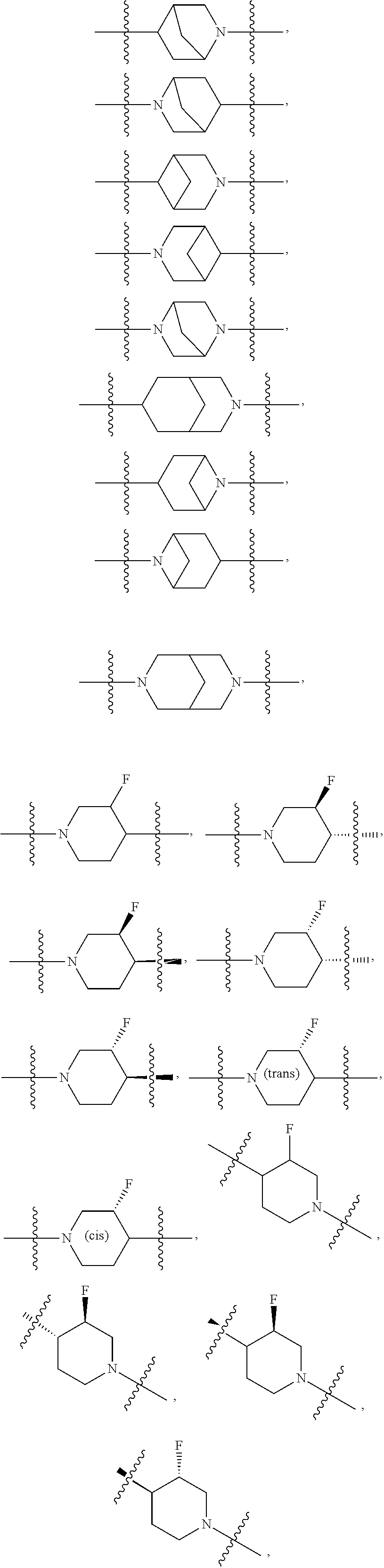

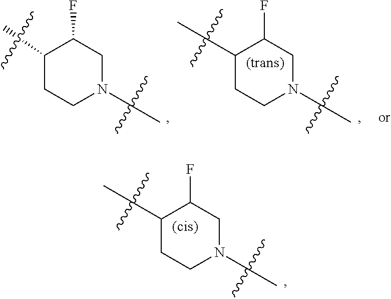

which, when substituted, is optionally further substituted with 0 to 4 (for example, 0, 1, 2, 3 or 4) substituents selected from H, F, CF₃, methyl, ═O, hydroxymethyl, COOH, CN or NH₂;
in some embodiments, L is selected from a bond,

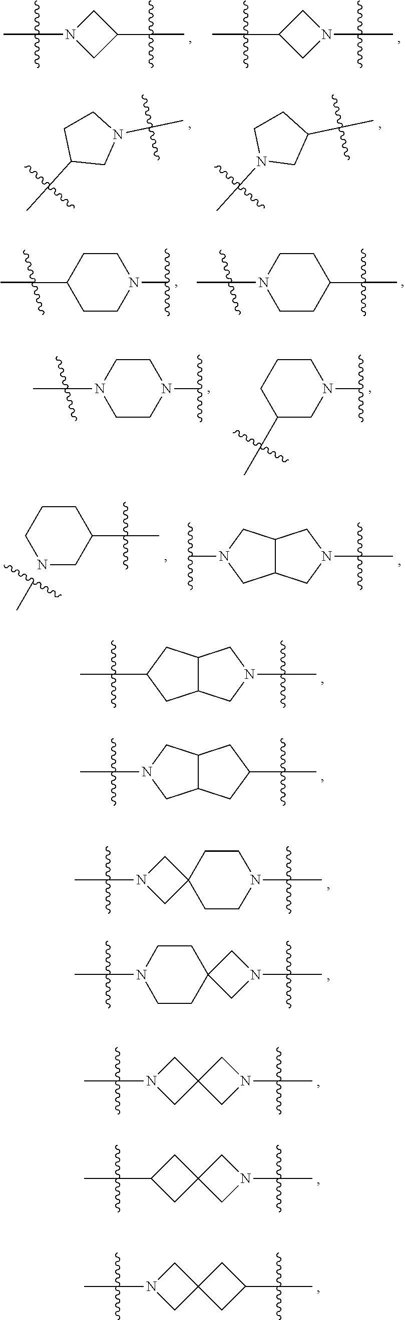

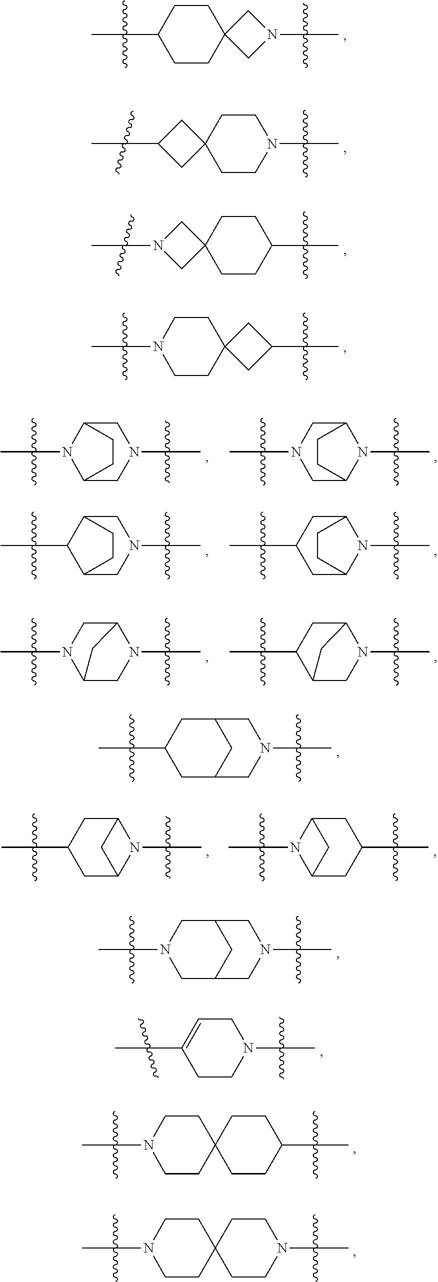

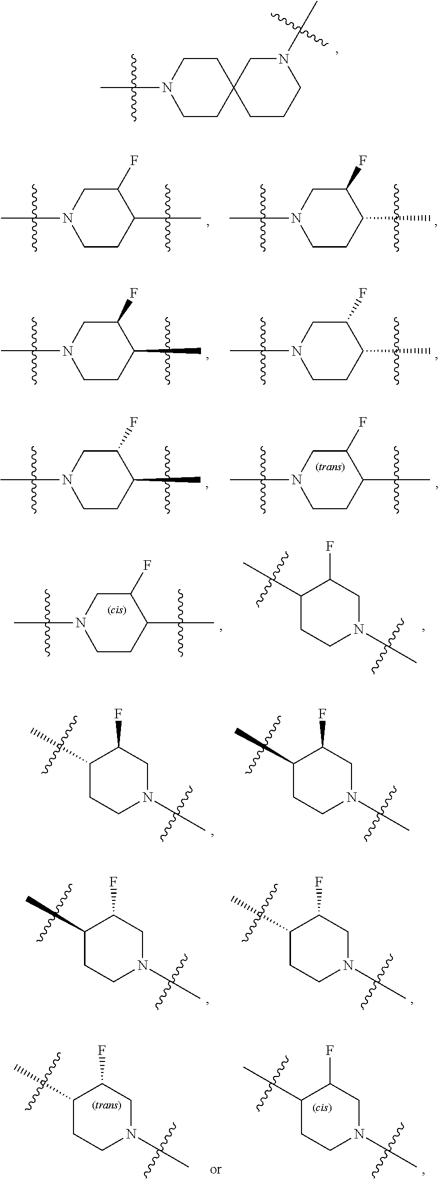

wherein the left side thereof is linked to B;
in some embodiments, L is selected from

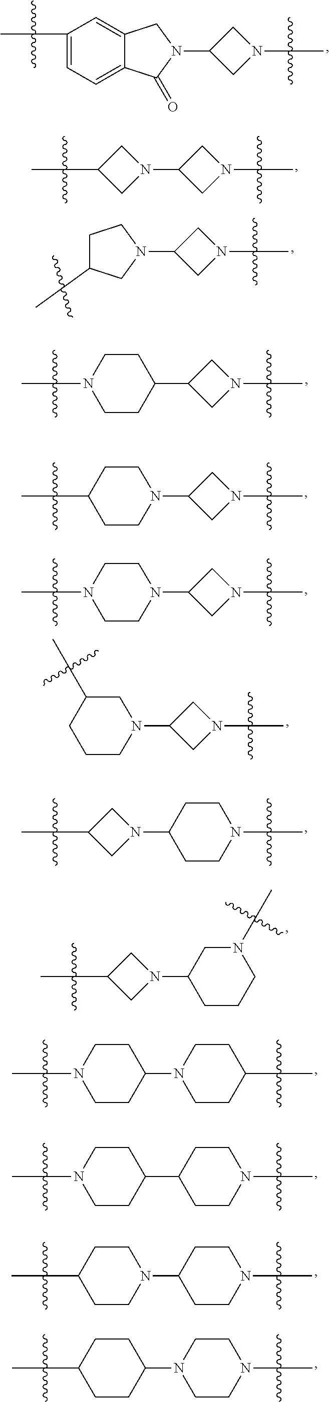

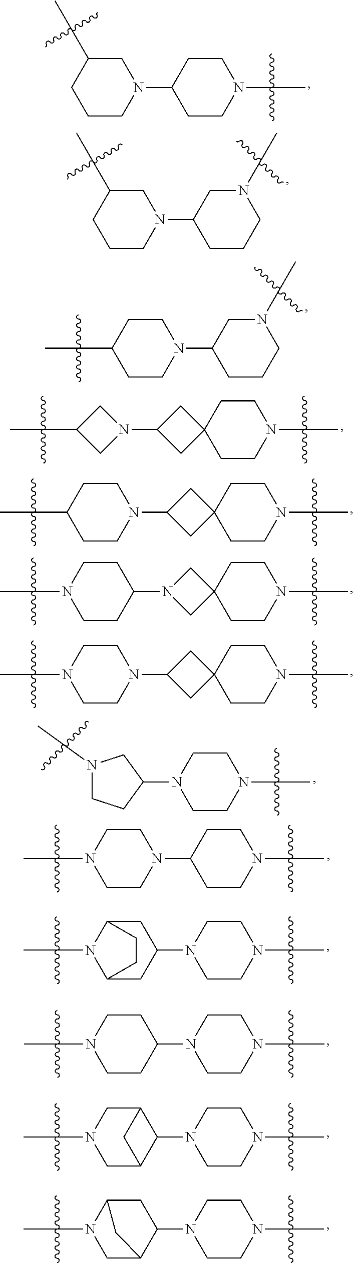

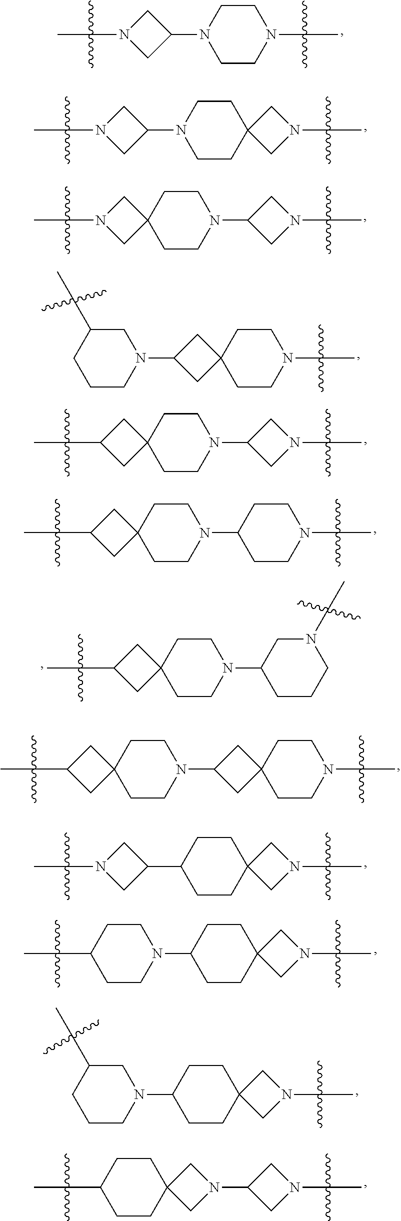

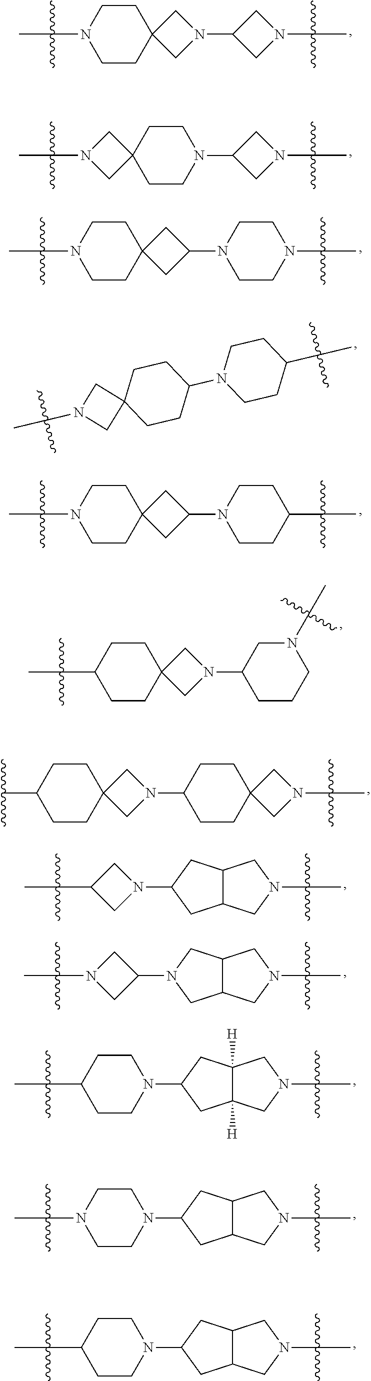

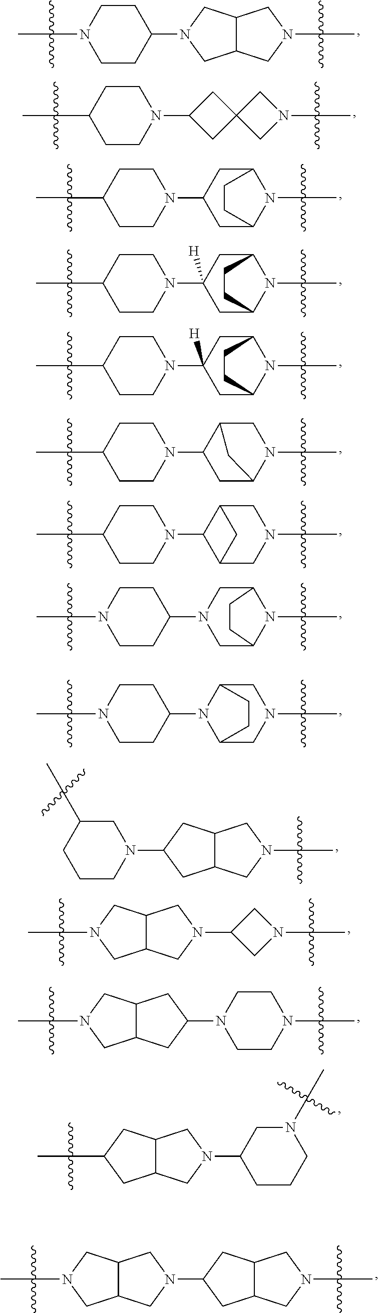

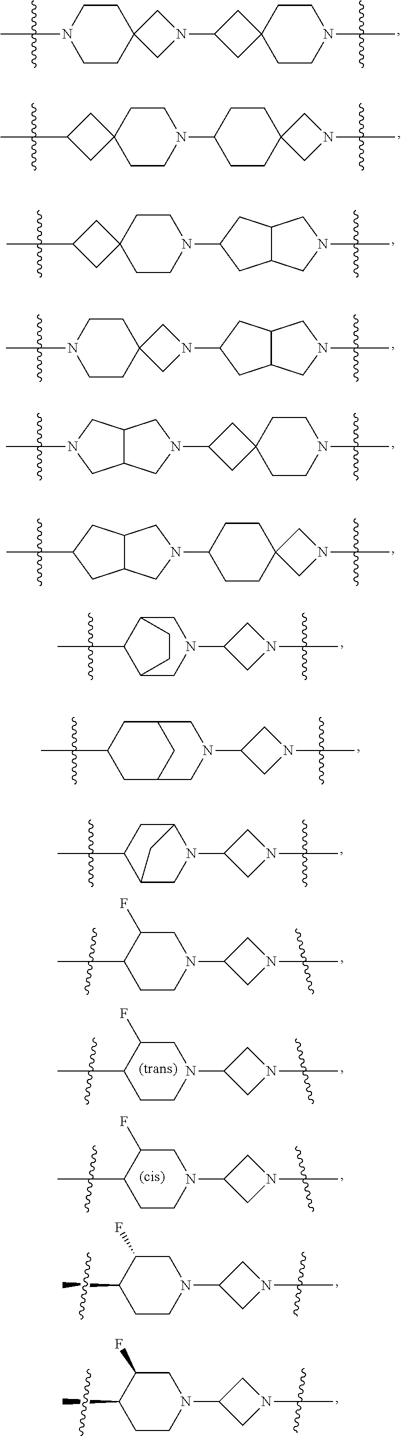

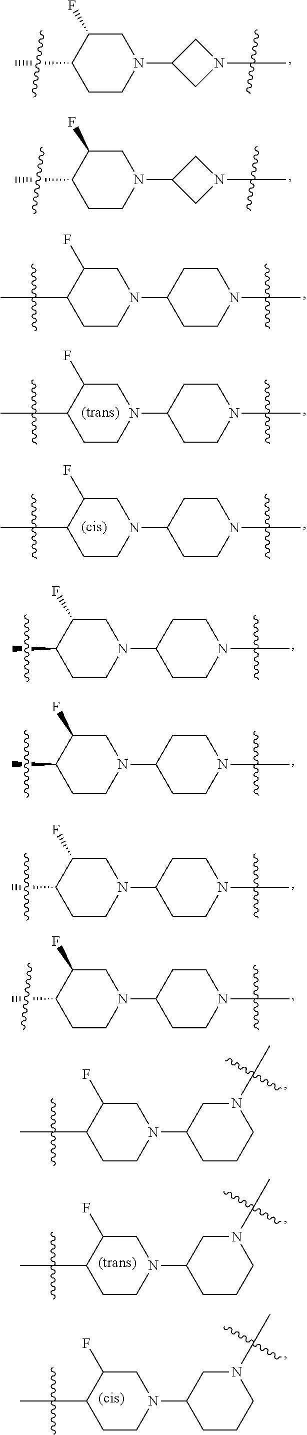

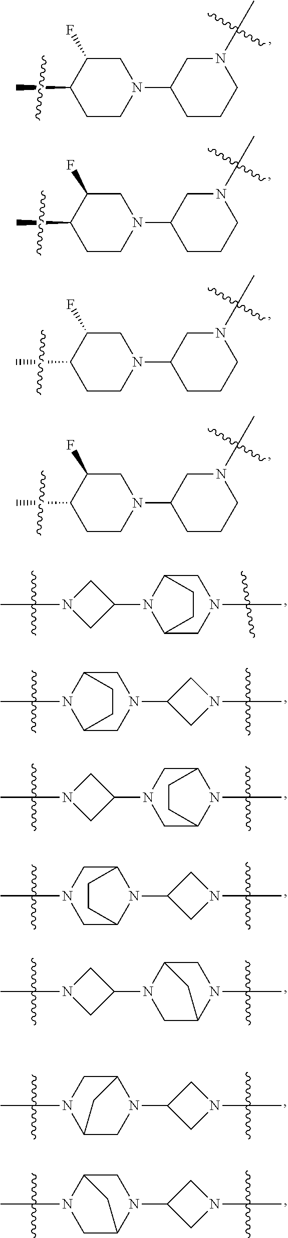

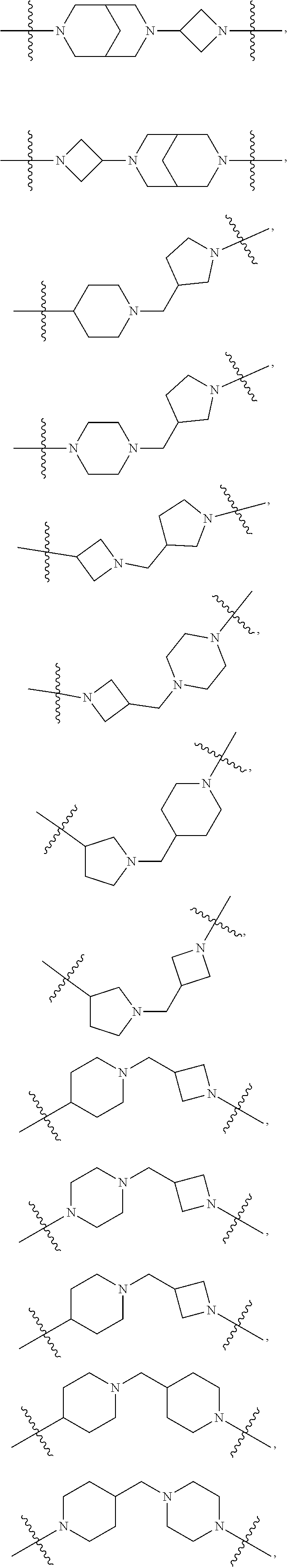

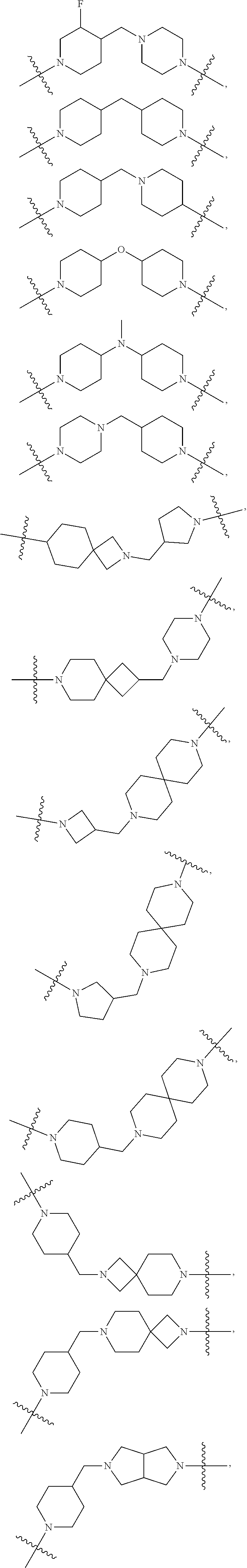

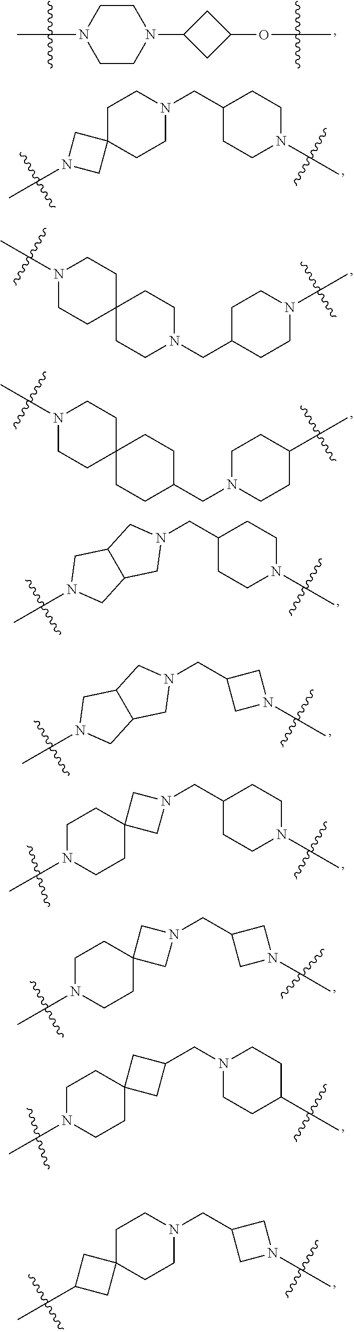

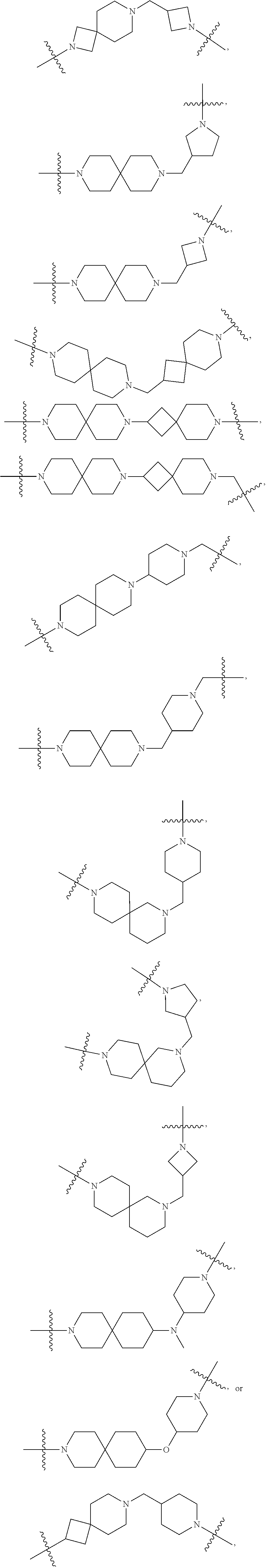

wherein the left side thereof is linked to B;
in some embodiments, L is selected from

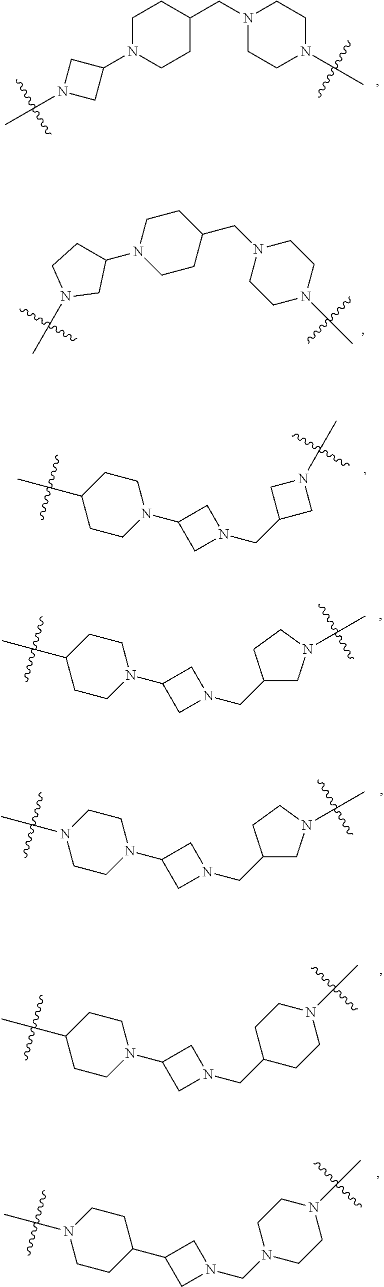

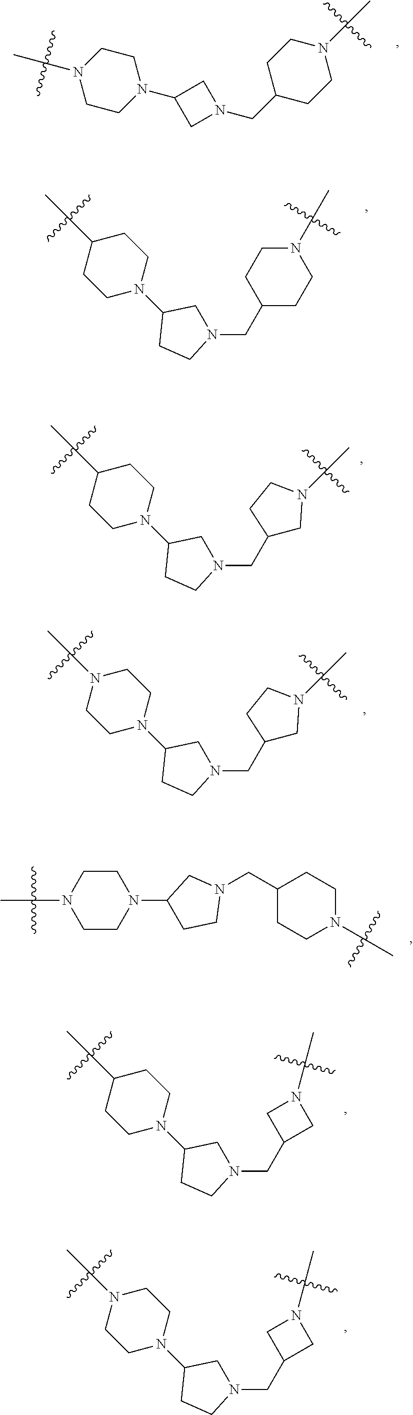

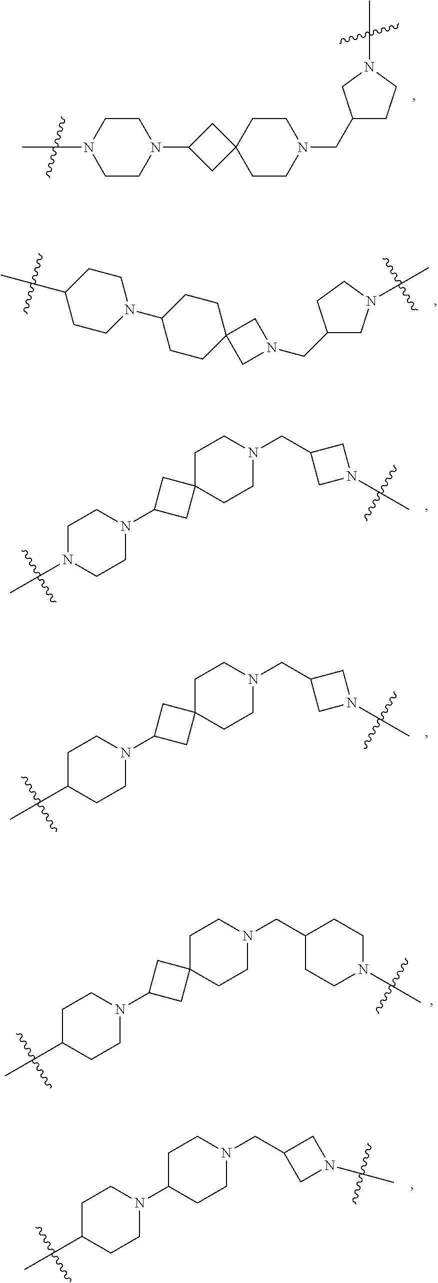 [SMILES: *N1CCN(C2CC3(CCN(CC4CCN(*)C4)CC3)C2)CC1 ; *C1CCN(C2CCC3(CC2)CN(CC2CCN(*)C2)C3)CC1 ; *N1CCN(C2CC3(CCN(CC4CN(*)C4)CC3)C2)CC1 ; *C1CCN(C2CC3(CCN(CC4CN(*)C4)CC3)C2)CC1 ; *C1CCN(C2CC3(CCN(CC4CCN(*)CC4)CC3)C2)CC1 ; *C1CCN(C2CCN(CC3CN(*)C3)CC2)CC1]

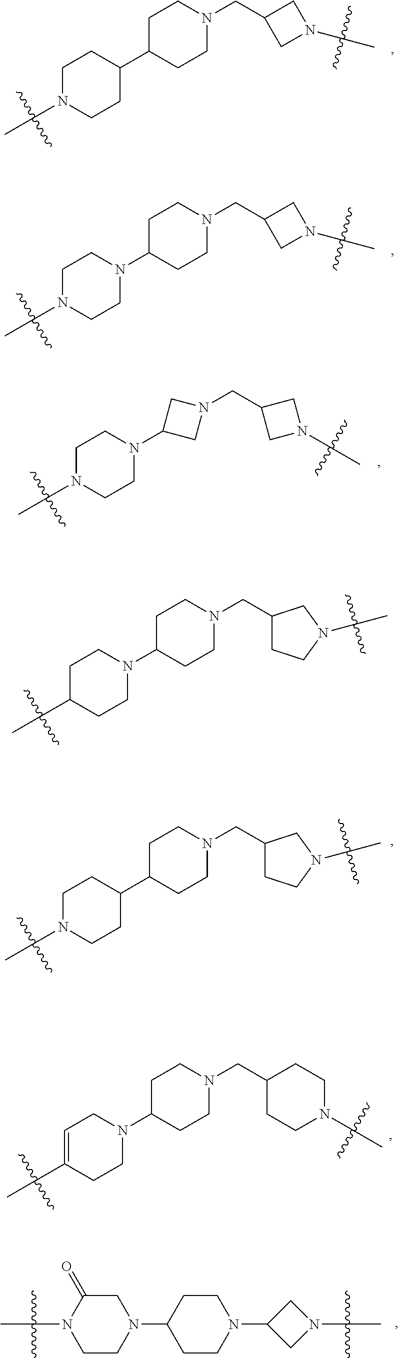

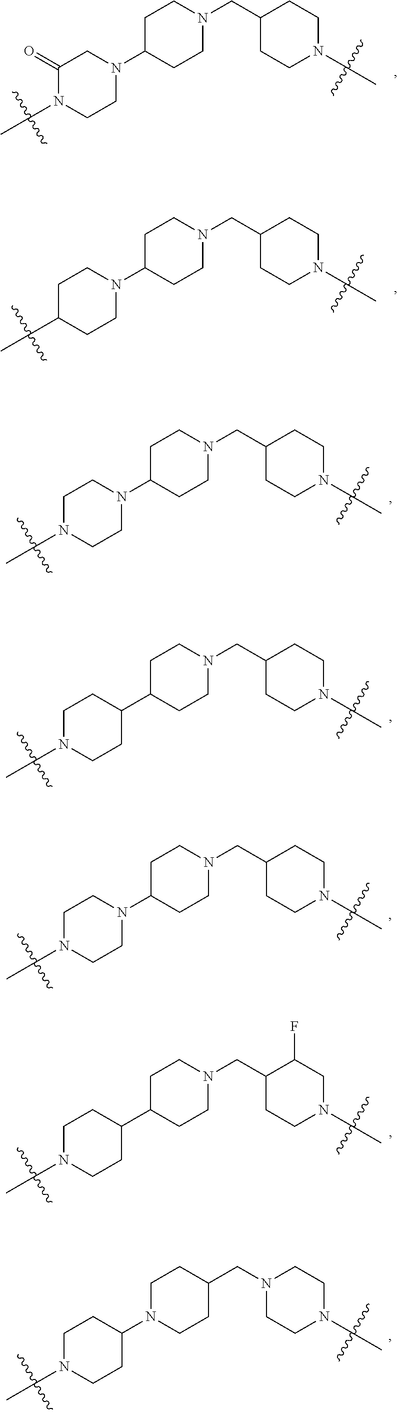

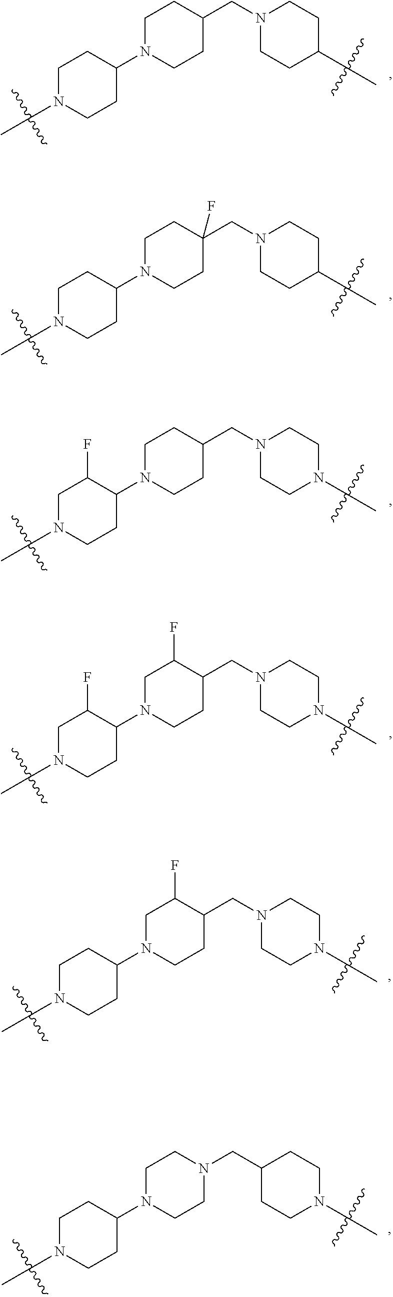 [SMILES: *C1CCN(CC2CCN(C3CCN(*)CC3)CC2)CC1 ; *C1CCN(CC2(F)CCN(C3CCN(*)CC3)CC2)CC1 ; *N1CCN(CC2CCN(C3CCN(*)CC3F)CC2)CC1 ; *N1CCN(CC2CCN(C3CCN(*)CC3F)CC2F)CC1 ; *N1CCC(N2CCC(CN3CCN(*)CC3)C(F)C2)CC1 ; *N1CCC(CN2CCN(C3CCN(*)CC3)CC2)CC1]

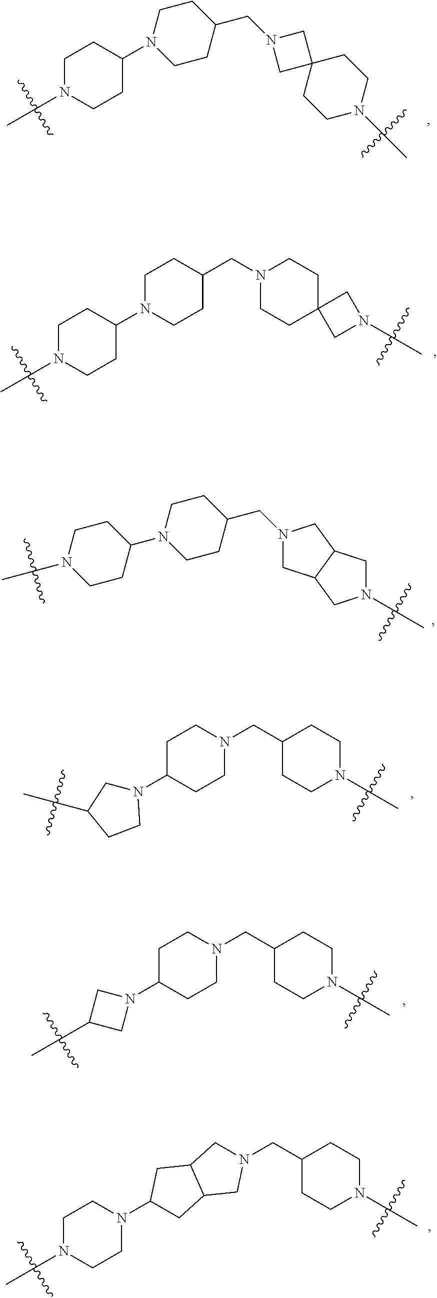 [SMILES: *N1CCC(N2CCC(CN3CC4(CCN(*)CC4)C3)CC2)CC1 ; *N1CCC(N2CCC(CN3CCC4(CC3)CN(*)C4)CC2)CC1 ; *N1CCC(N2CCC(CN3CC4CN(*)CC4C3)CC2)CC1 ; *C1CCN(C2CCN(CC3CCN(*)CC3)CC2)C1 ; *C1CN(C2CCN(CC3CCN(*)CC3)CC2)C1 ; *N1CCC(CN2CC3CC(N4CCN(*)CC4)CC3C2)CC1]

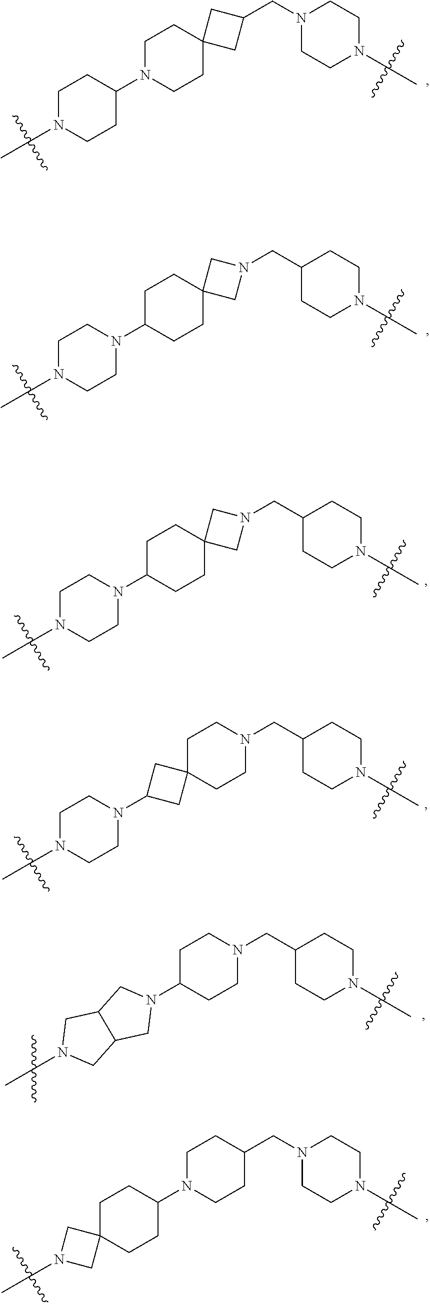 [SMILES: *N1CCC(N2CCC3(CC2)CC(CN2CCN(*)CC2)C3)CC1 ; *N1CCC(CN2CC3(CCC(N4CCN(*)CC4)CC3)C2)CC1 ; *N1CCC(CN2CC3(CCC(N4CCN(*)CC4)CC3)C2)CC1 ; *N1CCC(CN2CCC3(CC2)CC(N2CCN(*)CC2)C3)CC1 ; *N1CCC(CN2CCC(N3CC4CN(*)CC4C3)CC2)CC1 ; *N1CCN(CC2CCN(C3CCC4(CC3)CN(*)C4)CC2)CC1]

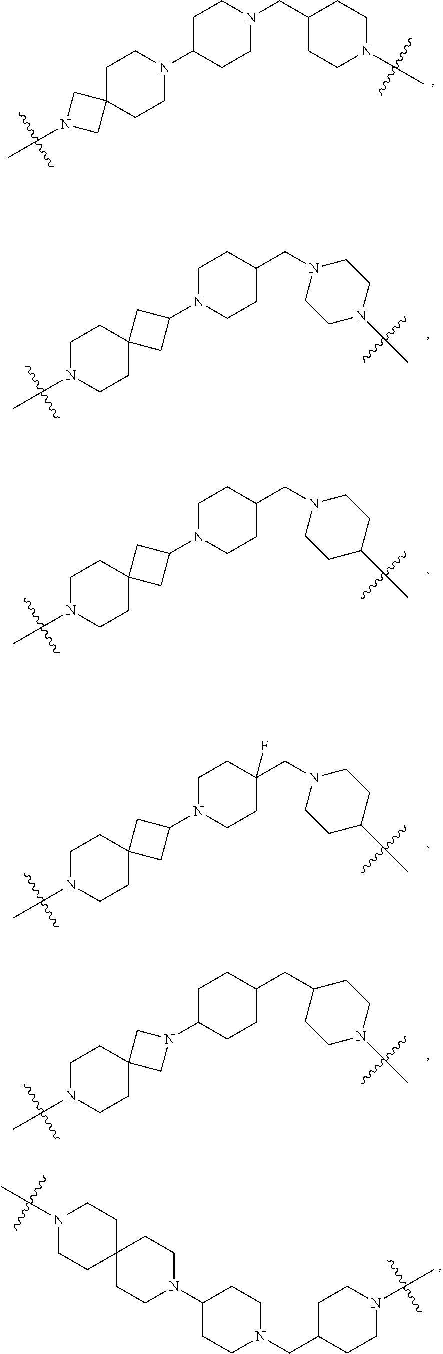 [SMILES: *N1CCC(CN2CCC(N3CCC4(CC3)CN(*)C4)CC2)CC1 ; *N1CCN(CC2CCN(C3CC4(CCN(*)CC4)C3)CC2)CC1 ; *C1CCN(CC2CCN(C3CC4(CCN(*)CC4)C3)CC2)CC1 ; *C1CCN(CC2(F)CCN(C3CC4(CCN(*)CC4)C3)CC2)CC1 ; *N1CCC(CC2CCC(N3CC4(CCN(*)CC4)C3)CC2)CC1 ; *N1CCC(CN2CCC(N3CCC4(CCN(*)CC4)CC3)CC2)CC1]

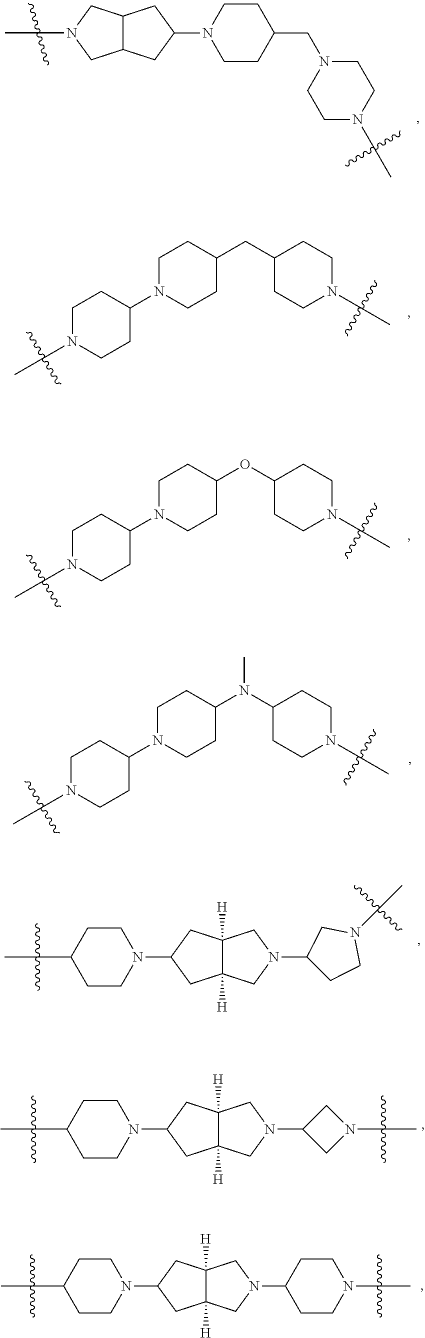

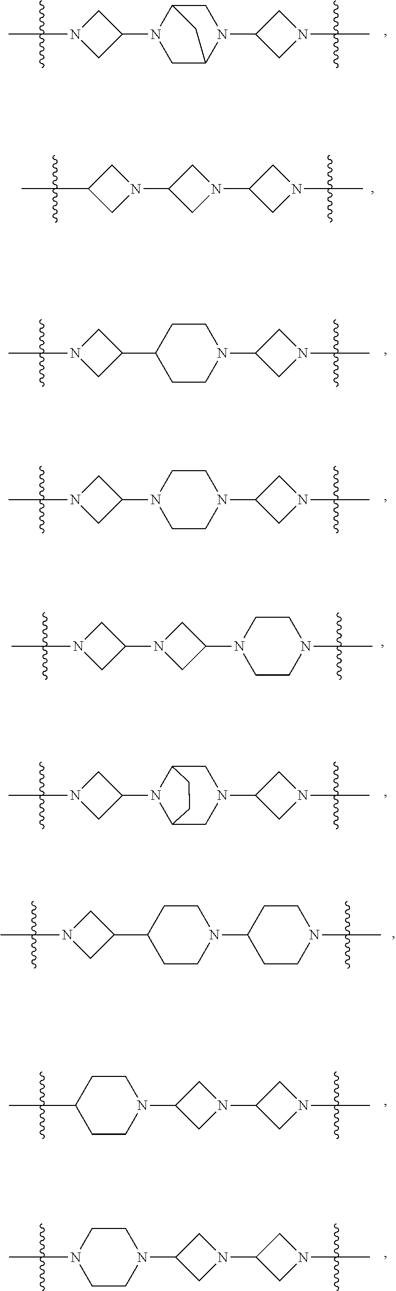

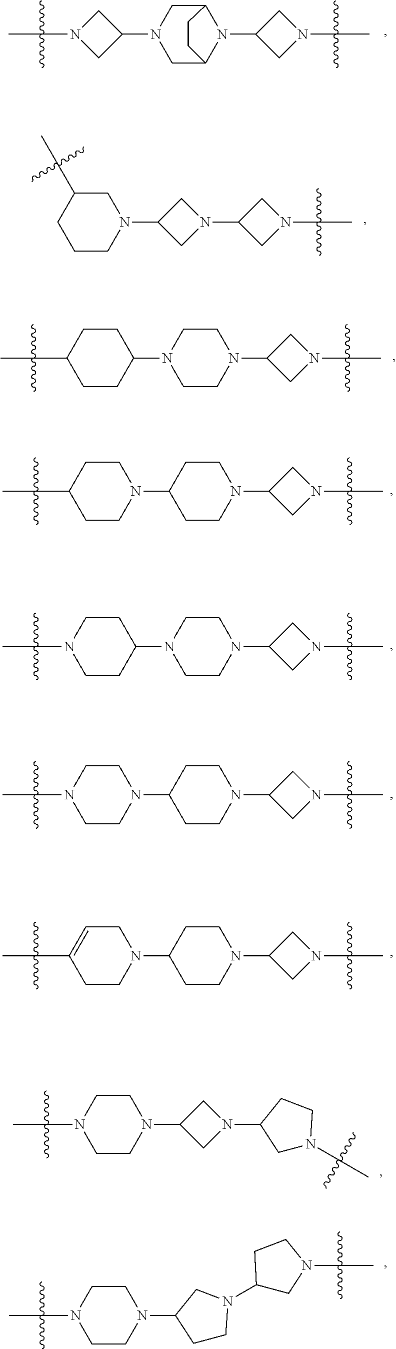

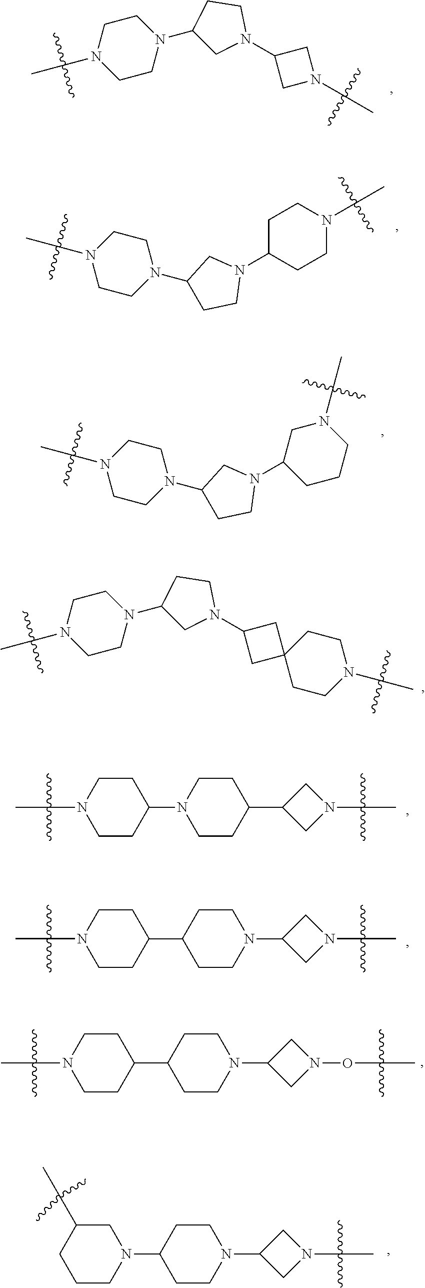

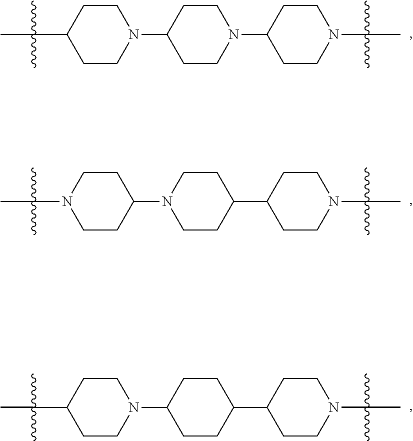 [SMILES: *C1CCN(C2CCN(C3CCN(*)CC3)CC2)CC1 ; *N1CCC(C2CCN(C3CCN(*)CC3)CC2)CC1 ; *C1CCN(C2CCC(C3CCN(*)CC3)CC2)CC1]

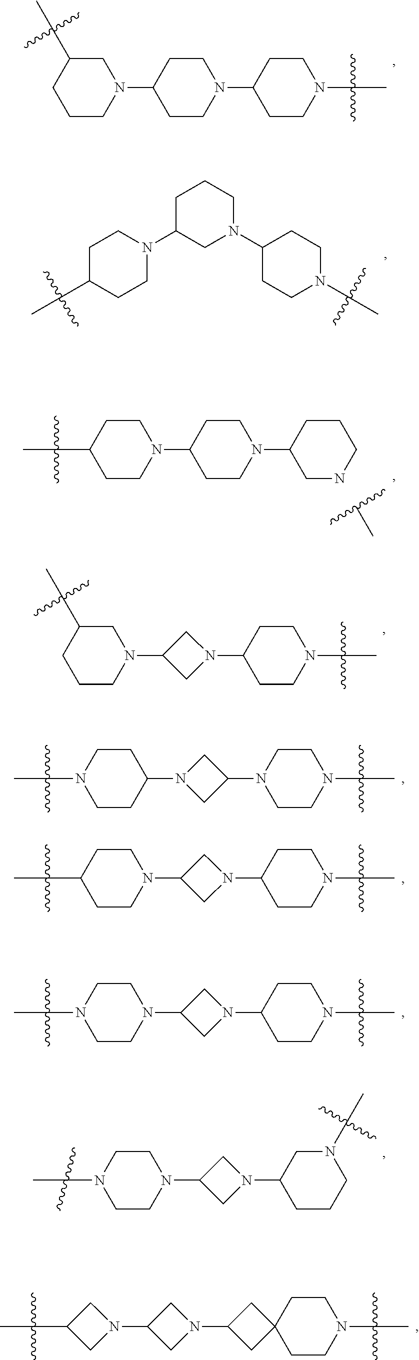

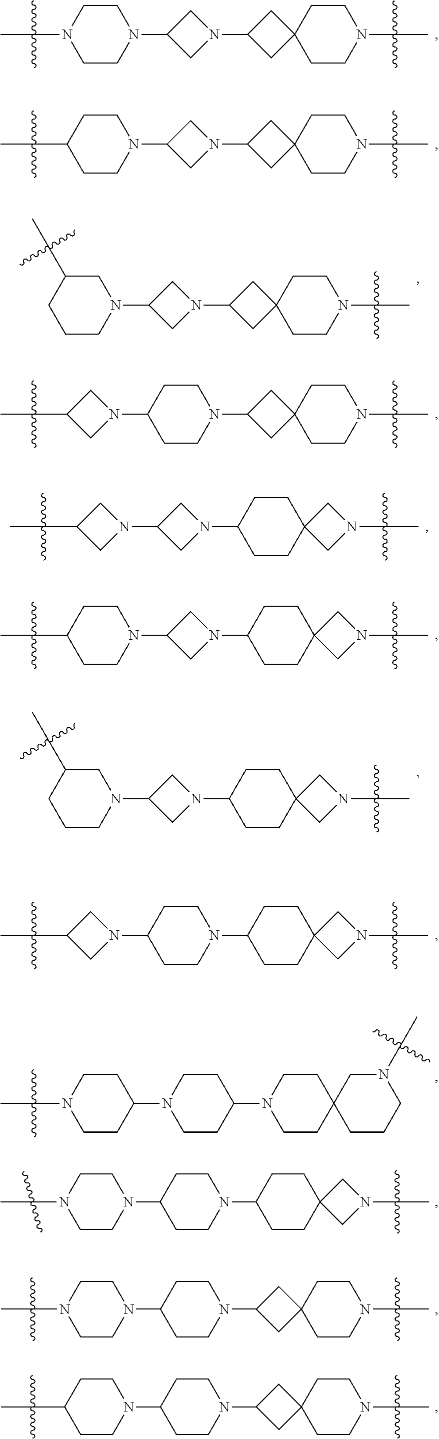

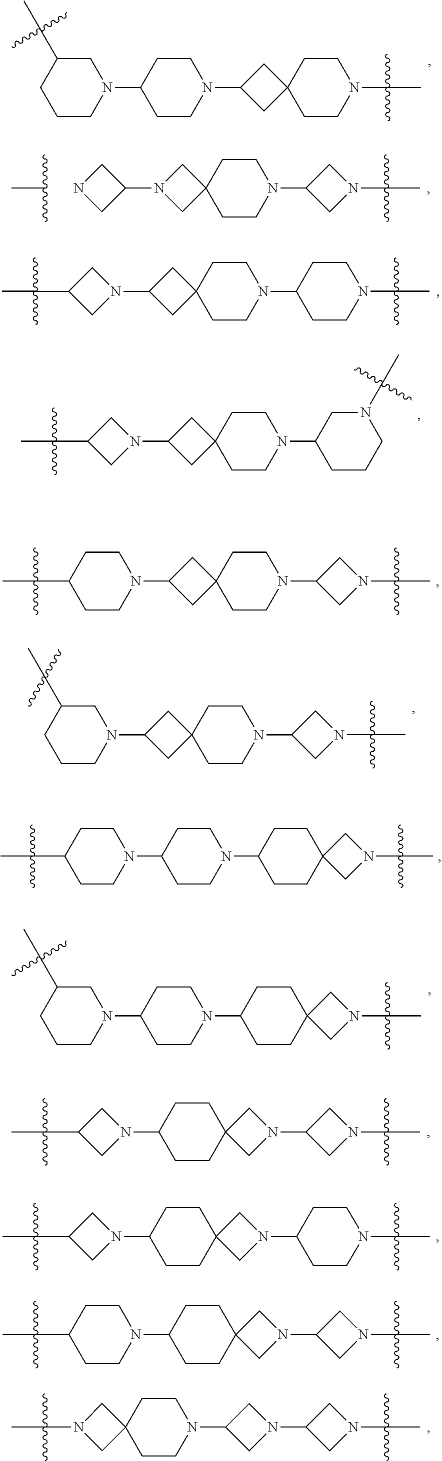

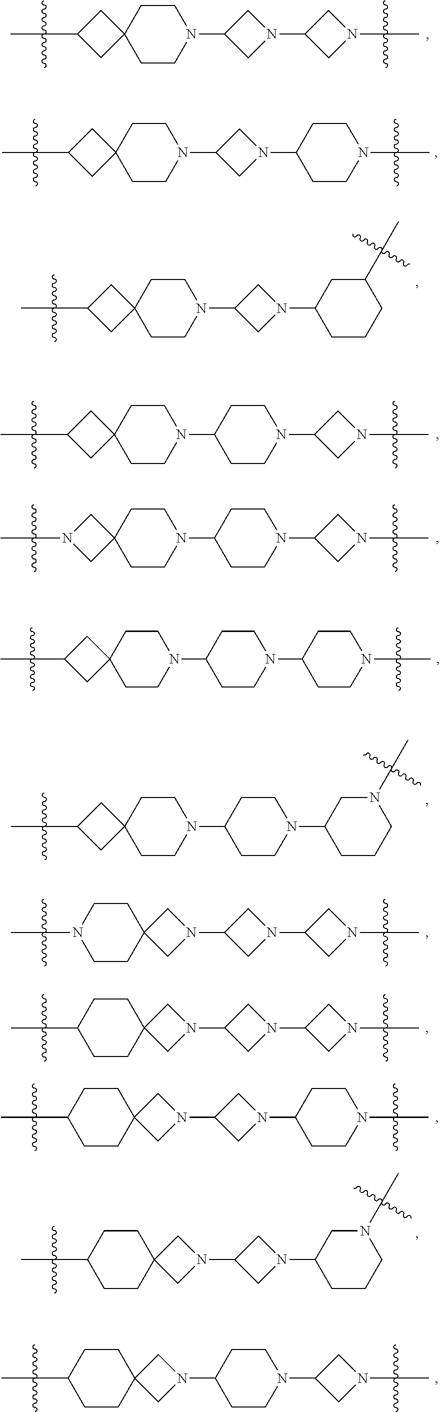

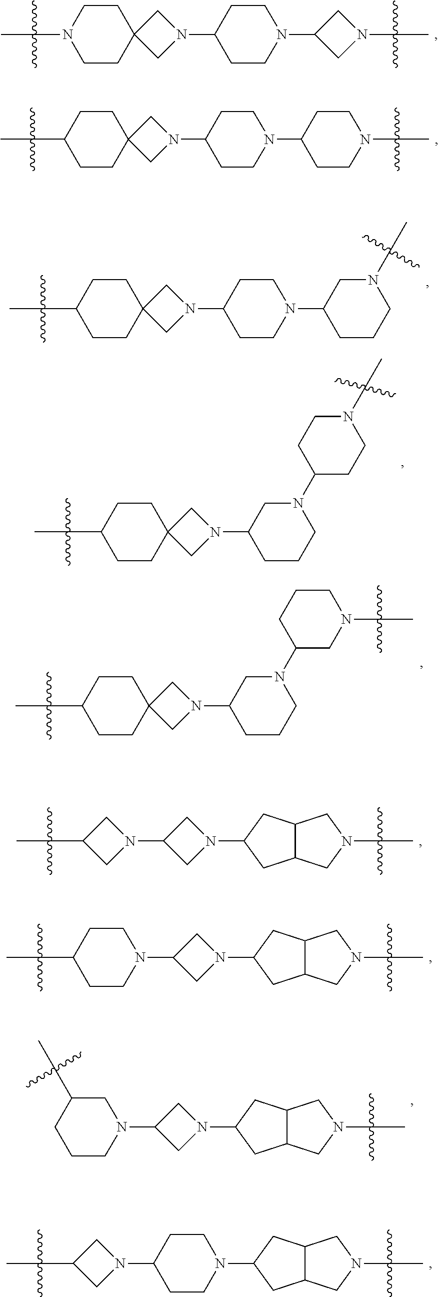

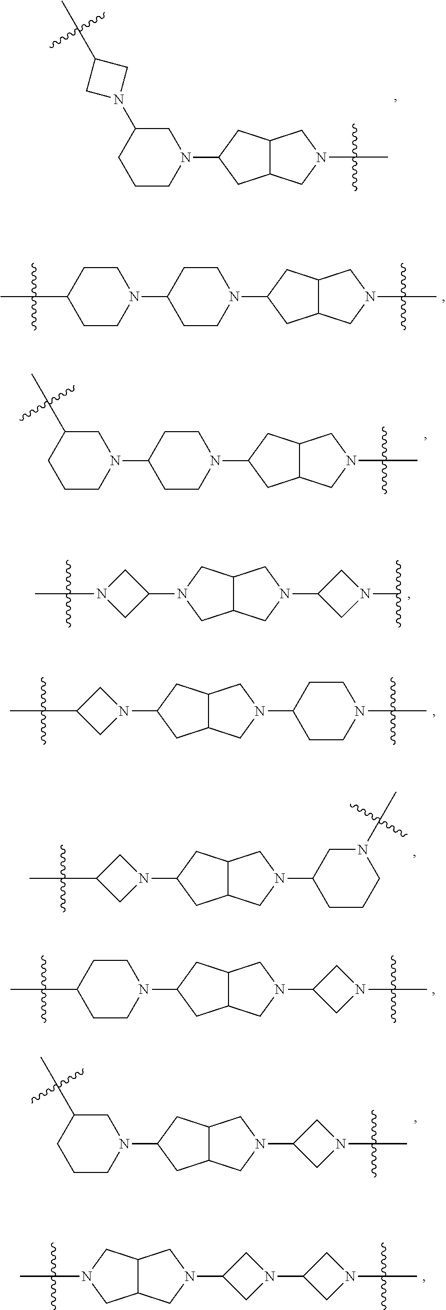

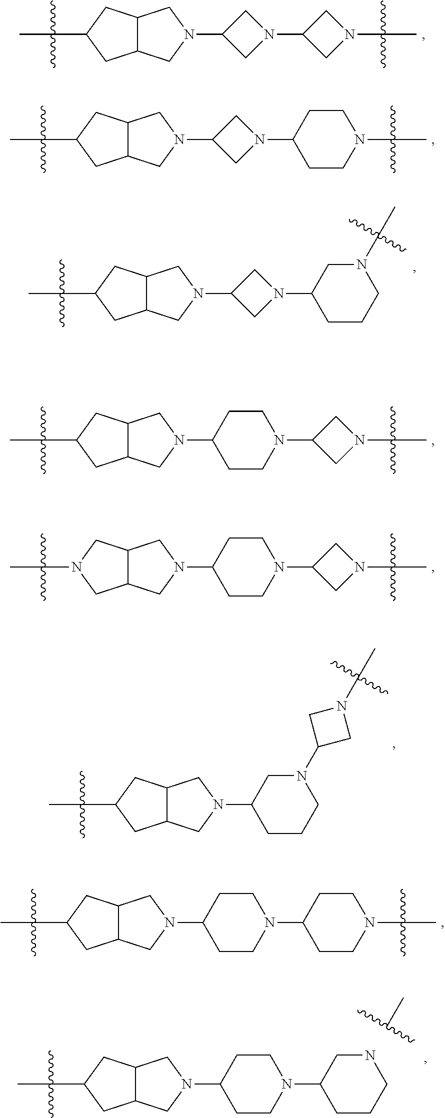

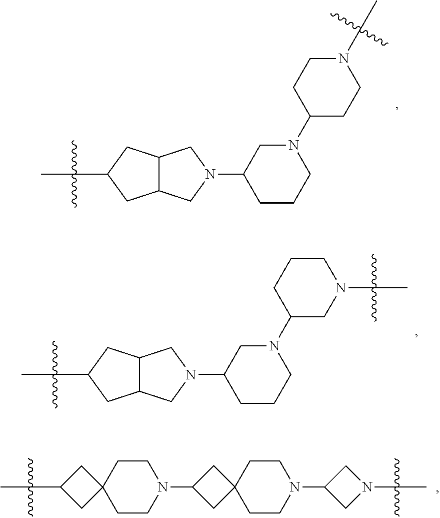 [SMILES: *C1CC2CN(C3CCCN(C4CCN(*)CC4)C3)CC2C1 ; *C1CC2CN(C3CCCN(C4CCCN(*)C4)C3)CC2C1 ; *C1CC2(CCN(C3CC4(CCN(C5CN(*)C5)CC4)C3)CC2)C1]

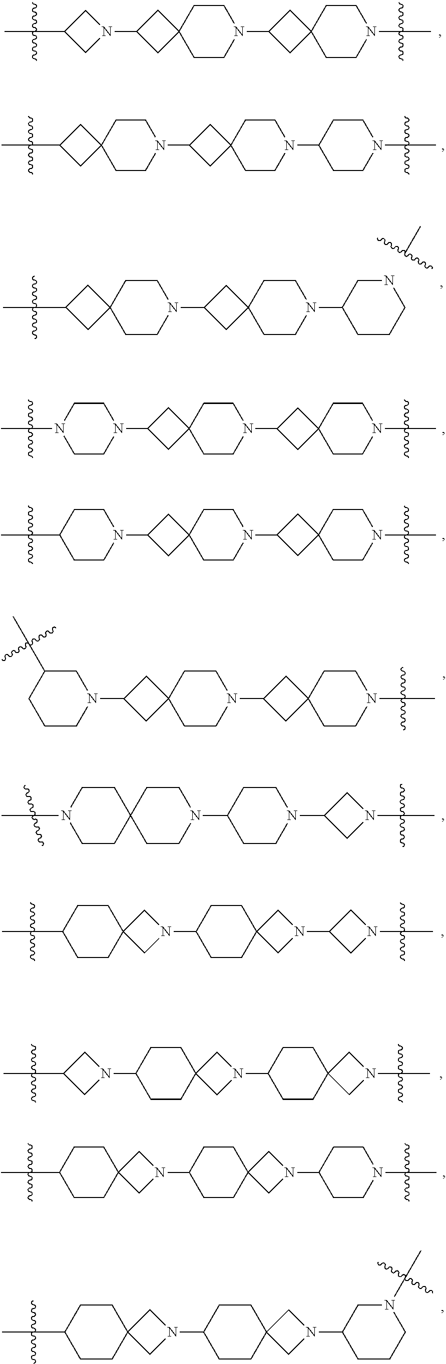

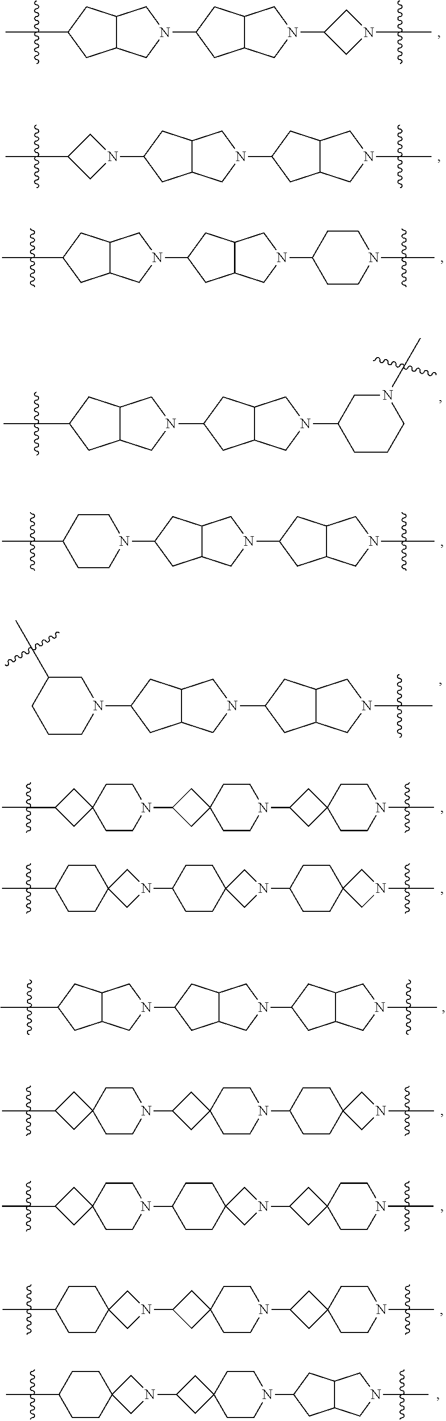

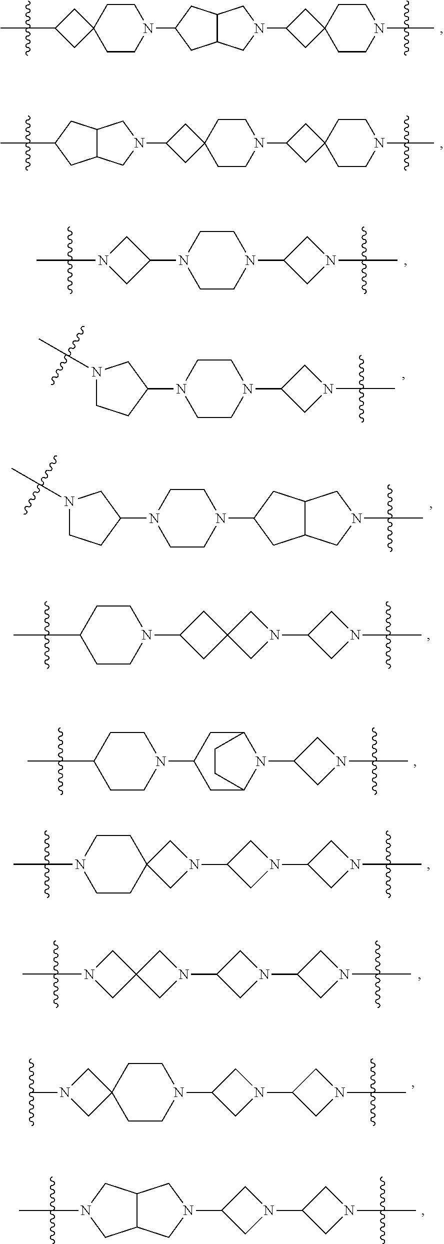

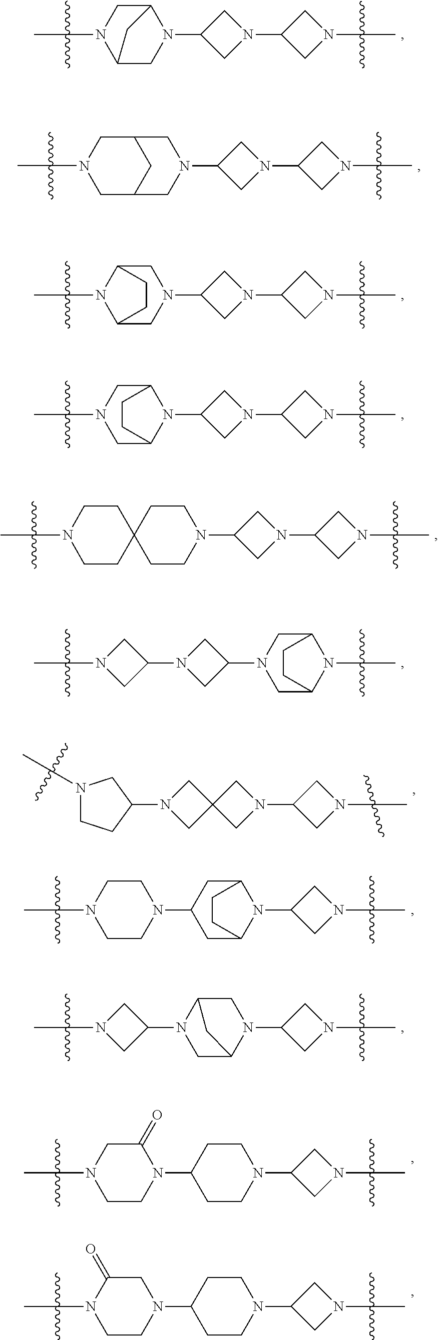

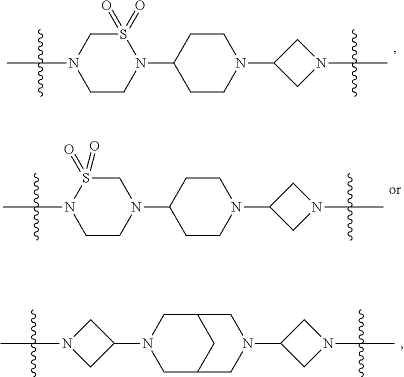 [SMILES: *N1CC(N2CCC(N3CCN(*)CS3(=O)=O)CC2)C1 ; *N1CC(N2CCC(N3CCN(*)S(=O)(=O)C3)CC2)C1 ; *N1CC(N2CC3CC(C2)CN(C2CN(*)C2)C3)C1]

wherein the left side thereof is linked to B;
in some embodiments, L is selected from

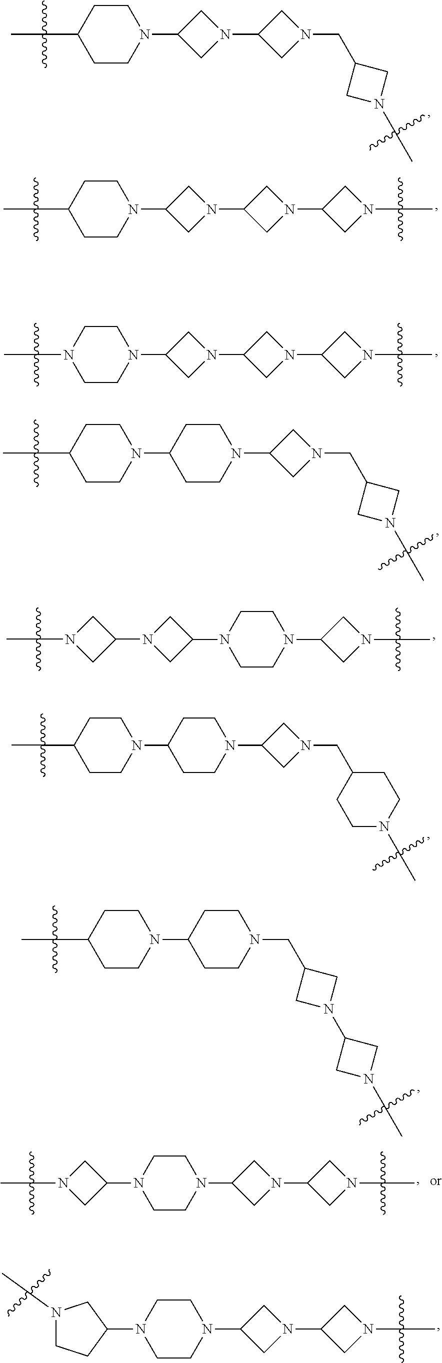

wherein the left side thereof is linked to B;
in some embodiments, L is selected from

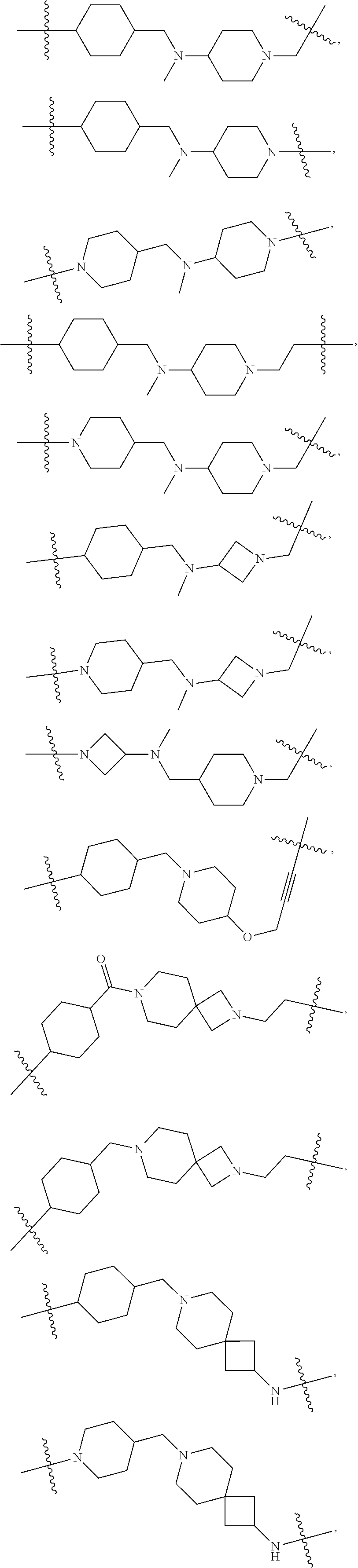

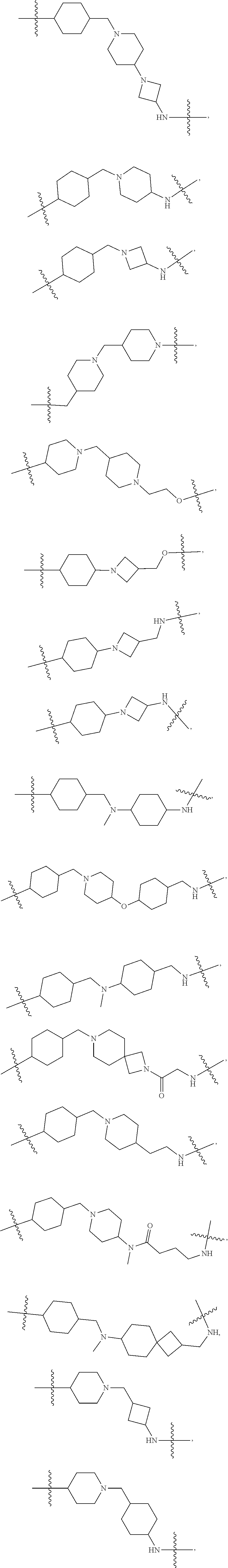

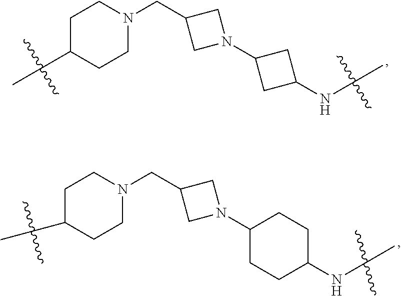 [SMILES: *NC1CC(N2CC(CN3CCC(*)CC3)C2)C1 ; *NC1CCC(N2CC(CN3CCC(*)CC3)C2)CC1]

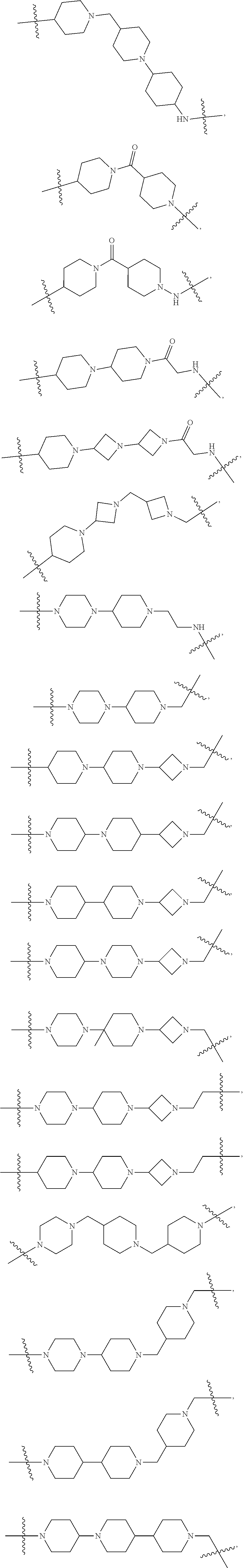

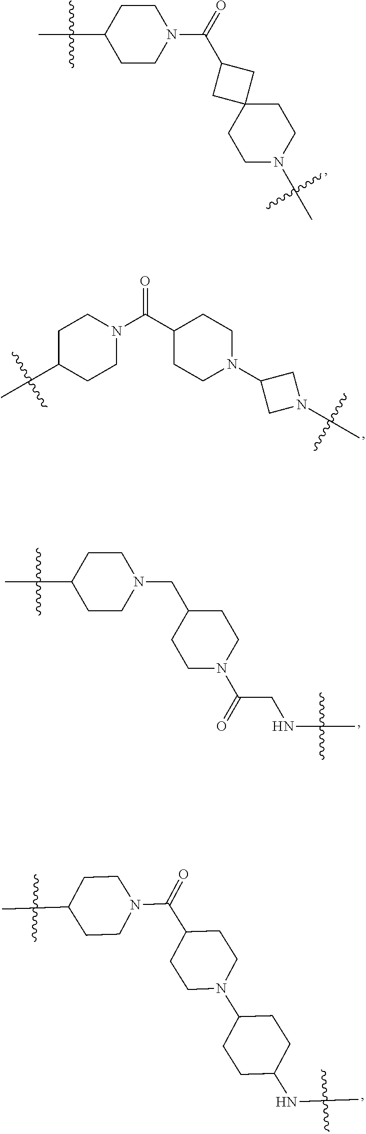

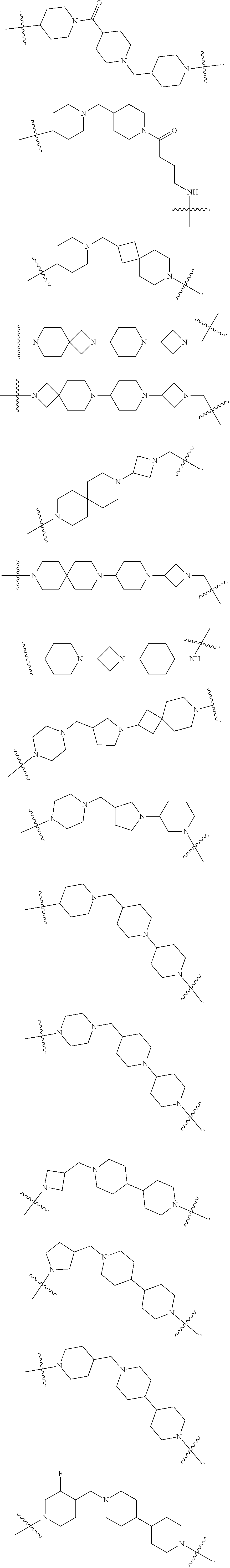

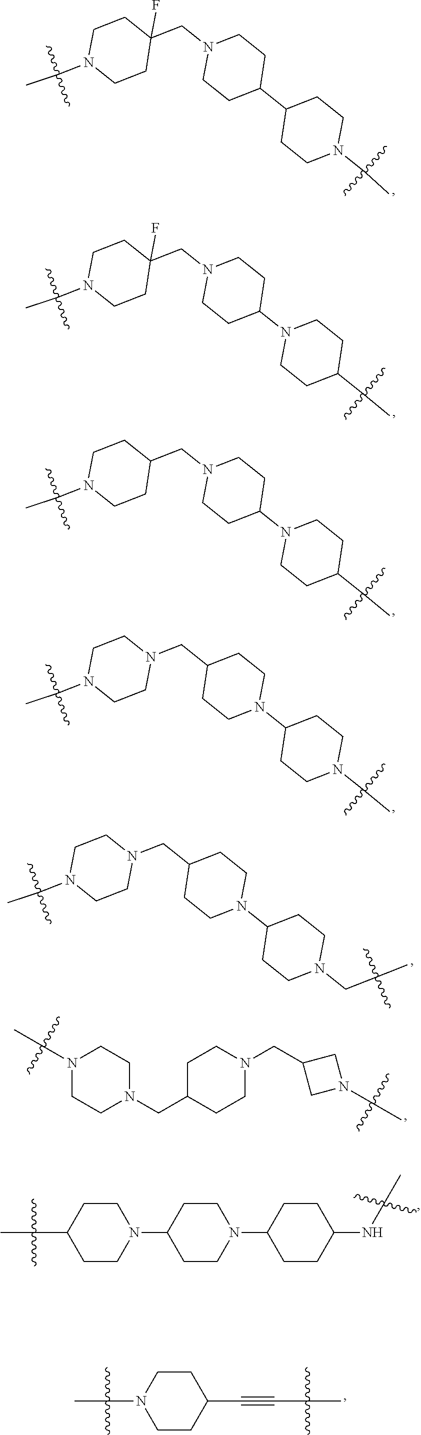

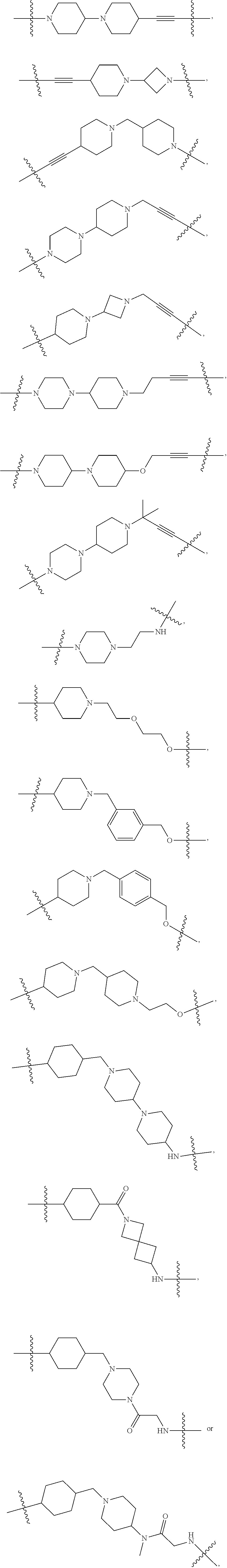

wherein the left side thereof is linked to B;
in some embodiments, L is selected from

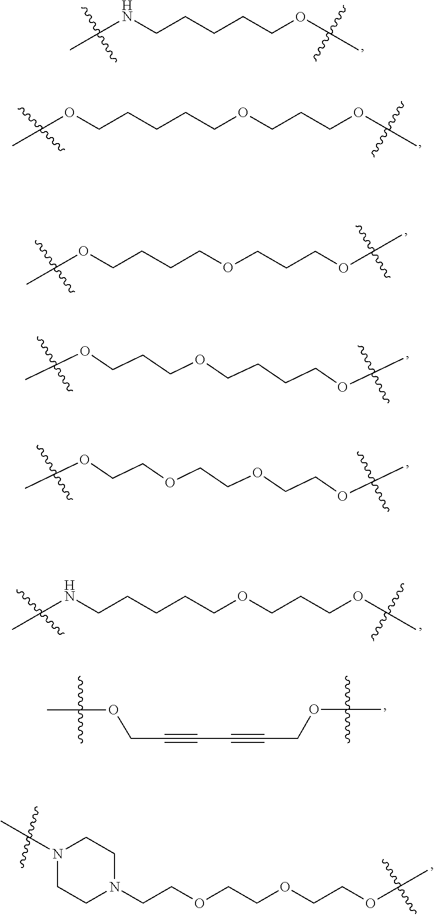

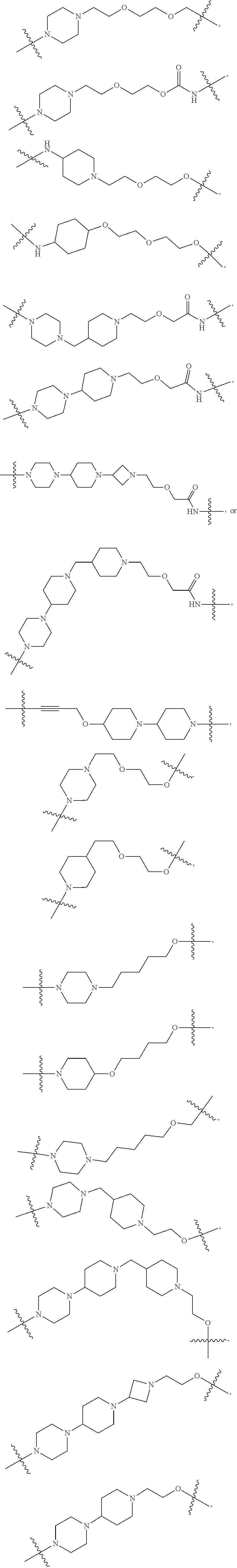

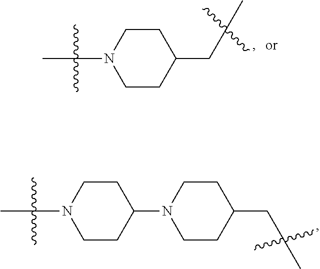 [SMILES: *CC1CCN(*)CC1 ; *CC1CCN(C2CCN(*)CC2)CC1]

wherein the left side thereof is linked to B;
in some embodiments, L is selected from one of the groups in Table L-1, wherein the left side thereof is linked to B;
in some embodiments, B is selected from

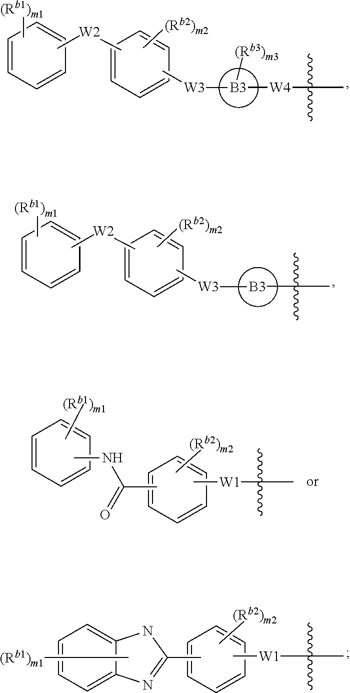

in some embodiments, B3 is selected from 5- to 6-membered heteroaryl, wherein the heteroaryl contains 1 to 4 (for example, 1, 2, 3 or 4) heteroatoms selected from O, S, or N;
in some embodiments, B3 is selected from pyrazolyl, oxazolyl, dioxazolyl, oxadiazolyl, imidazolyl, pyrrolyl, thienyl, thiazolyl, pyridyl, pyrazinyl, pyrimidinyl, or pyridazinyl;
in some embodiments, each W1 is independently selected from a bond, —O—, —S—, —NR^{w1}—, —NR^{w1}C(═O)—, —NR^{w1}S(═O)₂—, or —NR^{w1}S(═O)₂NR^{w1}—, wherein the left side thereof is directly linked to phenyl;
in some embodiments, each W1 is independently selected from a bond, —O—, —NH—, —NHC(═O)—, —NHS(═O)₂—, —NHS(═O)₂NH—, or —NHS(═O)₂N(CH₃)—, wherein the left side thereof is directly linked to phenyl;
in some embodiments, each W2 is independently selected from —NR^{w1}— or —(CR^{w2}R^{w3})ᵣ—, wherein the left side thereof is directly linked to phenyl;
in some embodiments, each W3 is independently selected from —O(CR^{w2}R^{w3})ₜ—, —S(CR^{w2}R^{w3})ₜ—, or —NR^{w1}(CR^{w2}R^{w3})ₜ—, wherein the left side thereof is directly linked to phenyl;
in some embodiments, W4 is selected from —O—, —S—, —NR^{w1}—, —NR^{w1}C(═O)—, —NR^{w1}S(═O)₂—, or —NR^{w1}S(═O)₂NR^{w1}—, wherein the left side thereof is directly linked to phenyl;
in some embodiments, each W4 is independently selected from —O—, —NH—, —NHC(═O)—, —NHS(═O)₂—, —NHS(═O)₂NH—, or —NHS(═O)₂N(CH₃)—, wherein the left side thereof is directly linked to phenyl;
in some embodiments, B is selected from

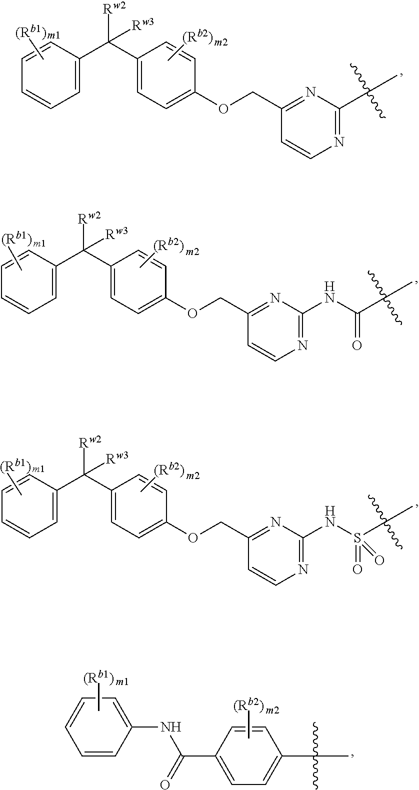

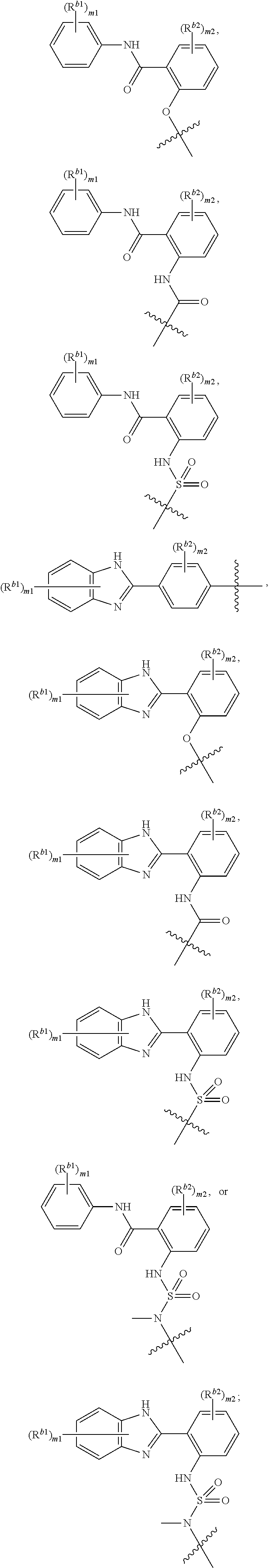

in some embodiments, B is selected from

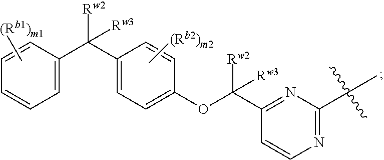

in some embodiments, each R^{w1} is independently selected from H, C₁₋₄ alkyl, or C₃₋₆ cycloalkyl;
in some embodiments, each R^{w1} is independently selected from H, methyl, ethyl, isopropyl, cyclopropyl or cyclobutyl;
in some embodiments, each R^{w2} or R^{w3} is independently selected from H, F, Cl, Br, I, OH, CN, COOH, C₁₋₄ alkyl, C₁₋₄ alkenyl, C₁₋₄ alkynyl, C₁₋₄ alkoxy, —N(R^{b21})₂, —C(═O)N(R^{b21})₂ or C₃₋₆ cycloalkyl, wherein the alkyl, alkoxy, alkenyl, alkynyl or cycloalkyl is optionally further substituted with 0 to 4 (for example, 0, 1, 2, 3 or 4) substituents selected from H, F, Cl, Br, I, OH, ═O, NH₂, CN, COOH, C₁₋₄ alkyl, C₁₋₄ alkoxy, halogen-substituted C₁₋₄ alkyl, or cyano-substituted C₁₋₄ alkyl;
in some embodiments, each R^{w2} or R^{w3} is independently selected from H, F, Cl, Br, I, OH, NH₂, CN, COOH, CONH₂, methyl, ethyl, isopropyl, ethenyl, ethynyl, methoxy, ethoxy, propoxy, cyclopropyl or cyclobutyl, wherein the methyl, ethyl, isopropyl, ethenyl, ethynyl, methoxy, ethoxy, propoxy, cyclopropyl or cyclobutyl is optionally further substituted with 0 to 4 (for example, 0, 1, 2, 3 or 4) substituents selected from H, F, Cl, Br, I, OH, or NH₂;
in some embodiments, R^{w2} and R^{w3} together with the carbon atoms to which they are attached form C₃₋₆ cycloalkyl or 3- to 8-membered heterocyclyl, wherein the cycloalkyl or heterocyclyl is optionally further substituted with 0 to 4 (for example, 0, 1, 2, 3 or 4) substituents selected from H, F, Cl, Br, I, OH, ═O, NH₂, CN, COOH, C₁₋₄ alkyl, C₁₋₄ alkoxy, halogen-substituted C₁₋₄ alkyl, or cyano-substituted C₁₋₄ alkyl, and the heterocyclyl contains 1 to 4 (for example, 1, 2, 3 or 4) heteroatoms selected from O, S, or N;
in some embodiments, R^{w2} and R^{w3} together with the carbon atoms to which they are attached form cyclopropyl, cyclobutyl, cyclopentyl, azetidinyl, oxetanyl, pyrrolidinyl, or piperidyl, wherein the cyclopropyl, cyclobutyl, cyclopentyl, azetidinyl, oxetanyl, pyrrolidinyl, or piperidyl is optionally further substituted with 0 to 4 (for example, 0, 1, 2, 3 or 4) substituents selected from H, F, Cl, Br, I, OH, NH₂, CN, CF₃, or CHF₂;
in some embodiments, each R^{b1} or R^{b2} is independently selected from H, F, Cl, Br, I, ═O, OH, CN, NO₂, COOH, C₁₋₄ alkyl, C₁₋₄ alkenyl, C₁₋₄ alkynyl, C₁₋₄ alkoxy, C₁₋₄ deuterated alkoxy, C₁₋₄ alkylthio, —(CH₂)ₙ—R^{b21}, —OR^{b21}, —N(R^{b21})₂, —C(═O)N(R^{b21})₂, —C(═O)OR^{b21}, —C(═O)R^{b22}, —NR^{b21}C(═O)R^{b22}, —NR^{b21}S(═O)₂R^{b22}, C₃₋₆ cycloalkyl, C₆₋₁₀ aryl, 5- to 10-membered heteroaryl or 4- to 10-membered heterocyclyl, wherein the alkyl, alkenyl, alkynyl, alkoxy, alkylthio, cycloalkyl, heterocyclyl, aryl or heteroaryl is optionally further substituted with 0 to 4 (for example, 0, 1, 2, 3 or 4) substituents selected from H, F, Cl, Br, I, OH, ═O, —N(R^{b21})₂, CN, COOH, C₁₋₄ alkyl, C₁₋₄ alkoxy, halogen-substituted C₁₋₄ alkyl, cyano-substituted C₁₋₄ alkyl, C₃₋₆ cycloalkyl, 5- to 10-membered heteroaryl or 4- to 10-membered heterocyclyl, and the heteroaryl or heterocyclyl contains 1 to 4 (for example, 1, 2, 3 or 4) heteroatoms selected from O, S, or N;
in some embodiments, each R^{b1} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, deuterated methoxy, methylthio, ethylthio, morpholinyl, piperazinyl, pyrrolidinyl, piperidyl or oxazolidinyl, wherein the methoxy, ethoxy, propoxy, isopropyloxy, methylthio, or ethylthio is optionally further substituted with 0 to 4 (for example, 0, 1, 2, 3 or 4) substituents selected from H, F, Cl, Br, I, OH, or NH₂;
in some embodiments, each R^{b1} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, OCD₃, methylthio, ethylthio, OCH₂CH₂Cl, or OCH₂CH₂CH₂Cl;
in some embodiments, each R^{b2} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, morpholinyl, piperazinyl, pyrrolidinyl, piperidyl or oxazolidinyl, wherein the methyl, ethyl, ethenyl, ethynyl, methoxy, ethoxy, propoxy, isopropyloxy, morpholinyl, piperazinyl, pyrrolidinyl, piperidyl or oxazolidinyl is optionally further substituted with 0 to 4 (for example, 0, 1, 2, 3 or 4) substituents selected from H, F, Cl, Br, I, OH, or NH₂;
in some embodiments, each R^{b3} is independently selected from H, F, Cl, Br, I, ═O, OH, CN, COOH, C₁₋₄ alkyl, C₁₋₄ alkenyl, C₁₋₄ alkynyl, C₁₋₄ alkoxy, C₁₋₄ alkylthio, —(CH₂)ₙ—R^{b21}, —OR^{b21}, —N(R^{b21})₂, —C(═O)N(R^{b21})₂, —NR^{b21}C(═O)R^{b22}, —S(═O)₂N(R^{b21})₂, —NR^{b21}S(═O)₂R^{b22}, —C(═O)R^{b22}, or —S(═O)₂R^{b22} wherein the alkyl, alkoxy, alkenyl, alkynyl or alkylthio is optionally further substituted with 0 to 4 (for example, 0, 1, 2, 3 or 4) substituents selected from H, F, Cl, Br, I, OH, ═O, —N(R^{b21})₂, CN, COOH, C₁₋₄ alkyl, C₁₋₄ alkoxy, halogen-substituted C₁₋₄ alkyl, or cyano-substituted C₁₋₄ alkyl;
in some embodiments, each R^{b3} is independently selected from H, F, Cl, Br, I, ═O, CF₃, CHF₂, OH, CN, COOH, NH₂, CONH₂, S(═O)₂NH₂, S(═O)₂CH₃, methyl, ethyl, isopropyl, ethenyl, ethynyl, methoxy, ethoxy, propoxy, methylthio or ethylthio, wherein the methyl, ethyl, isopropyl, ethenyl, ethynyl, methoxy, ethoxy, propoxy, methylthio or ethylthio is optionally further substituted with 0 to 4 (for example, 0, 1, 2, 3 or 4) substituents selected from H, F, Cl, Br, I, OH, or NH₂;
in some embodiments, each R^{b21} is independently selected from H, C₁₋₄ alkyl, C₃₋₆ cycloalkyl, C₆₋₁₀ aryl, 5- to 10-membered heteroaryl or 4- to 10-membered heterocyclyl, wherein the alkyl, alkoxy, cycloalkyl, aryl, heteroaryl or heterocyclyl is optionally further substituted with 0 to 4 (for example, 0, 1, 2, 3 or 4) substituents selected from H, F, Cl, Br, I, OH, ═O, NH₂, CN, CF₃, COOH, C₁₋₄ alkyl, C₃₋₆ cycloalkyl, or C₁₋₄ alkoxy, and the heteroaryl or heterocyclyl contains 1 to 4 (for example, 1, 2, 3 or 4) heteroatoms selected from O, S, or N;
in some embodiments, each R^{b21} is independently selected from H, C₁₋₃ alkyl, C₃₋₆ cycloalkyl, C₆₋₁₀ aryl, 5- to 6-membered heteroaryl or 4- to 6-membered heterocyclyl, wherein the alkyl, alkoxy, cycloalkyl, aryl, heteroaryl or heterocyclyl is optionally further substituted with 0 to 4 (for example, 0, 1, 2, 3 or 4) substituents selected from H, F, Cl, Br, I, OH, ═O, NH₂, CN, CF₃, COOH, C₁₋₄ alkyl, C₃₋₆ cycloalkyl, or C₁₋₄ alkoxy, and the heteroaryl or heterocyclyl contains 1 to 4 (for example, 1, 2, 3 or 4) heteroatoms selected from O, S, or N;
in some embodiments, each R^{b21} is independently selected from H, methyl, ethyl, isopropyl, cyclopropyl, cyclobutyl, pyrrolidinyl, piperidyl, morpholinyl, or piperazinyl, wherein the methyl, ethyl, isopropyl, cyclopropyl, cyclobutyl, pyrrolidinyl, piperidyl, morpholinyl, or piperazinyl is optionally further substituted with 0 to 4 (for example, 0, 1, 2, 3 or 4) substituents selected from H, F, Cl, or Br;
in some embodiments, each R^{b22} is independently selected from H, C₁₋₄ alkyl, C₁₋₄ alkenyl, C₁₋₄ alkynyl, C₁₋₄ alkoxy, or C₃₋₆ cycloalkyl, wherein the alkyl, alkoxy, cycloalkyl, alkenyl, or alkynyl is optionally further substituted with 0 to 4 (for example, 0, 1, 2, 3 or 4) substituents selected from H, F, Cl, Br, I, OH, ═O, NH₂, CN, CF₃, COOH, C₁₋₄ alkyl, C₃₋₆ cycloalkyl, or C₁₋₄ alkoxy;
in some embodiments, each R^{b22} is independently selected from H, methyl, ethyl, isopropyl, ethenyl, ethynyl, methoxy, ethoxy, propoxy, isopropyloxy, cyclopropyl, or cyclobutyl, wherein the methyl, ethyl, isopropyl, ethenyl, ethynyl, methoxy, ethoxy, propoxy, isopropyloxy, cyclopropyl, or cyclobutyl is optionally further substituted with 0 to 4 (for example, 0, 1, 2, 3 or 4) substituents selected from H, F, Cl, or Br;
in some embodiments, B is selected from

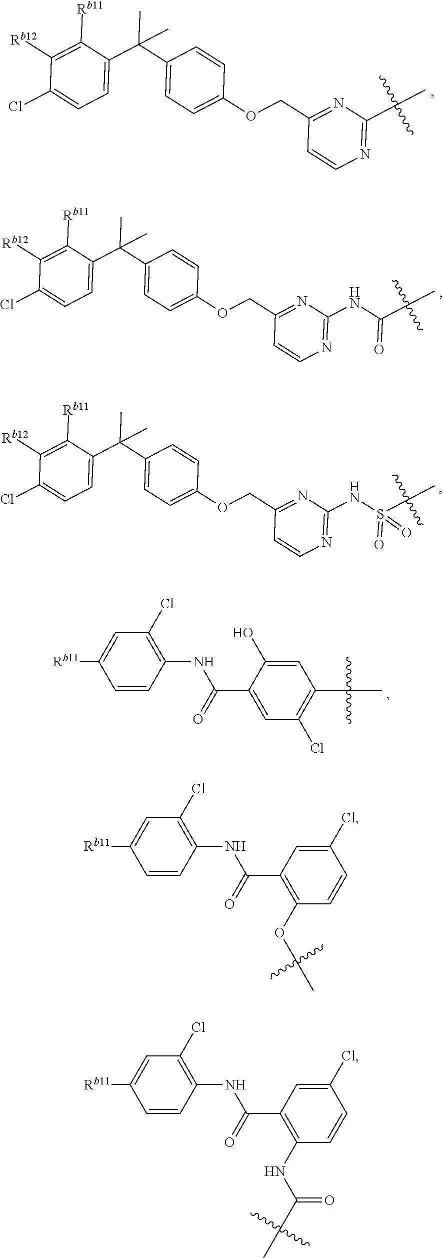

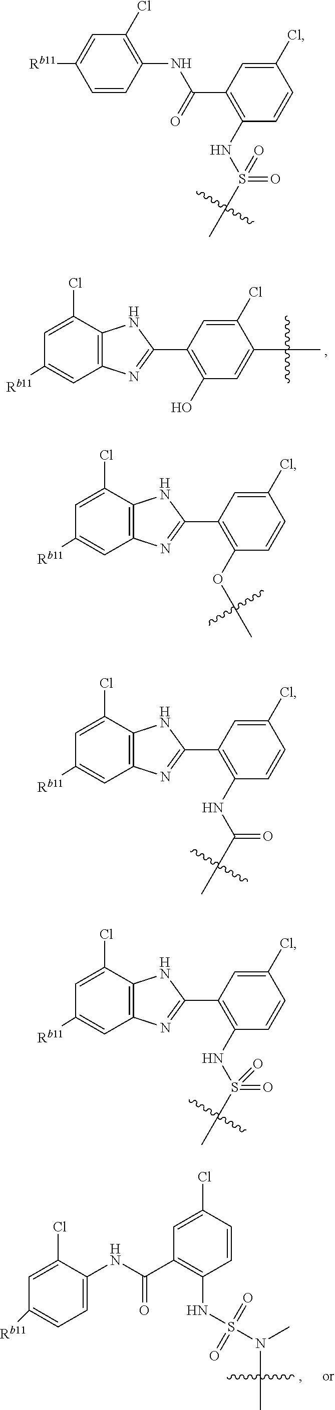

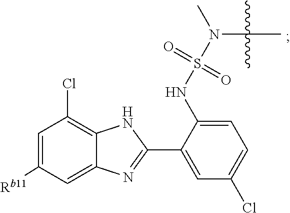

in some embodiments, B is selected from

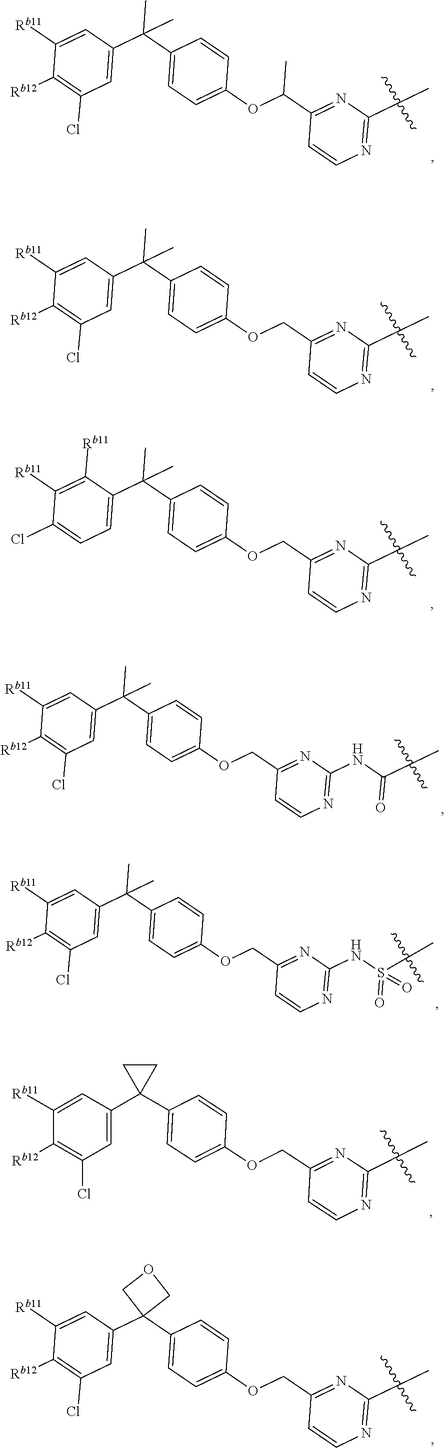

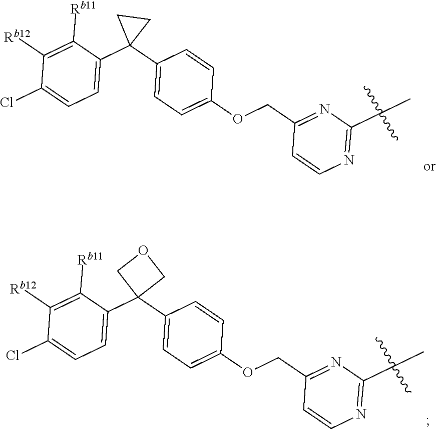

in some embodiments, each R^{b11} is independently selected from F, Cl, Br, CF₃, CN, or NO₂;
in some embodiments, each R^{b12} is independently selected from H, OCH₃, OCD₃, OCH₂CH₃, or OCH₂CH₂Cl;
in some embodiments, each R^{b12} is independently selected from H, OCH₃, OCH₂CH₃, OCH₂CH₂Cl, or OCH₂CH₂CH₂Cl;
in some embodiments, B is selected from

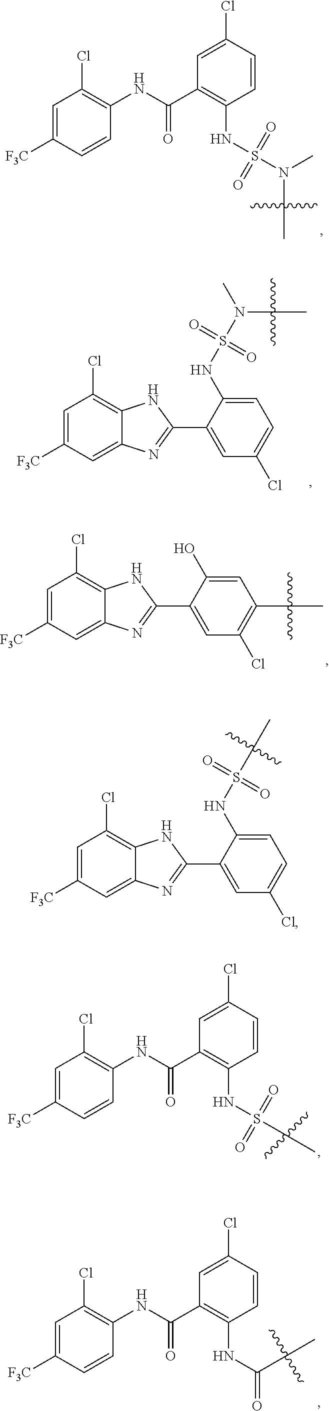 [SMILES: *N(C)S(=O)(=O)Nc1ccc(Cl)cc1C(=O)Nc1ccc(C(F)(F)F)cc1Cl ; *N(C)S(=O)(=O)Nc1ccc(Cl)cc1-c1nc2cc(C(F)(F)F)cc(Cl)c2[nH]1 ; *c1cc(O)c(-c2nc3cc(C(F)(F)F)cc(Cl)c3[nH]2)cc1Cl ; *S(=O)(=O)Nc1ccc(Cl)cc1-c1nc2cc(C(F)(F)F)cc(Cl)c2[nH]1 ; *S(=O)(=O)Nc1ccc(Cl)cc1C(=O)Nc1ccc(C(F)(F)F)cc1Cl ; *C(=O)Nc1ccc(Cl)cc1C(=O)Nc1ccc(C(F)(F)F)cc1Cl]

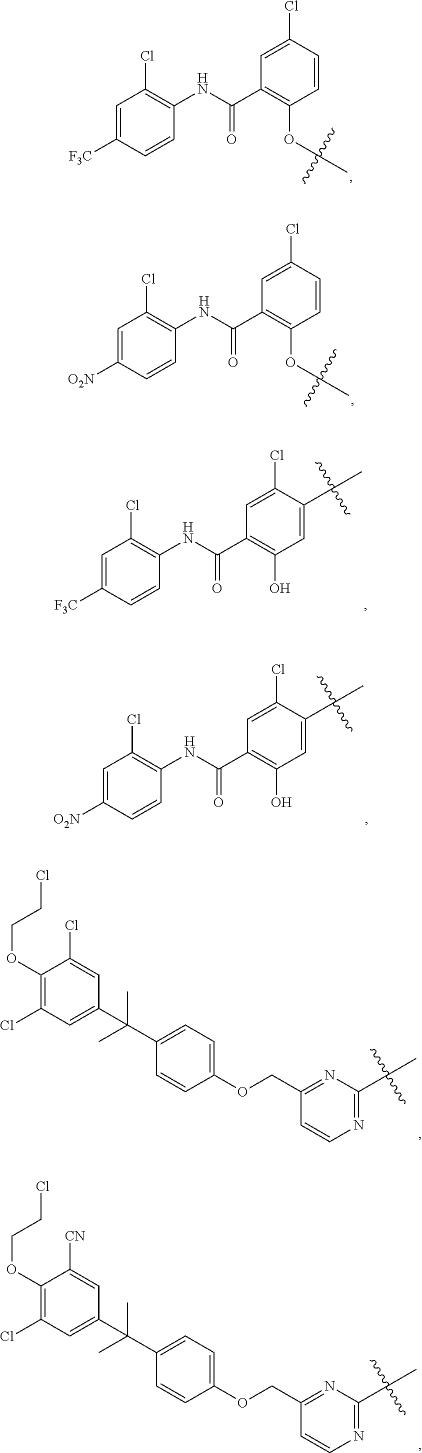 [SMILES: *Oc1ccc(Cl)cc1C(=O)Nc1ccc(C(F)(F)F)cc1Cl ; *Oc1ccc(Cl)cc1C(=O)Nc1ccc([N+](=O)[O-])cc1Cl ; *c1cc(O)c(C(=O)Nc2ccc(C(F)(F)F)cc2Cl)cc1Cl ; *c1cc(O)c(C(=O)Nc2ccc([N+](=O)[O-])cc2Cl)cc1Cl ; *c1nccc(COc2ccc(C(C)(C)c3cc(Cl)c(OCCCl)c(Cl)c3)cc2)n1 ; *c1nccc(COc2ccc(C(C)(C)c3cc(Cl)c(OCCCl)c(C#N)c3)cc2)n1]

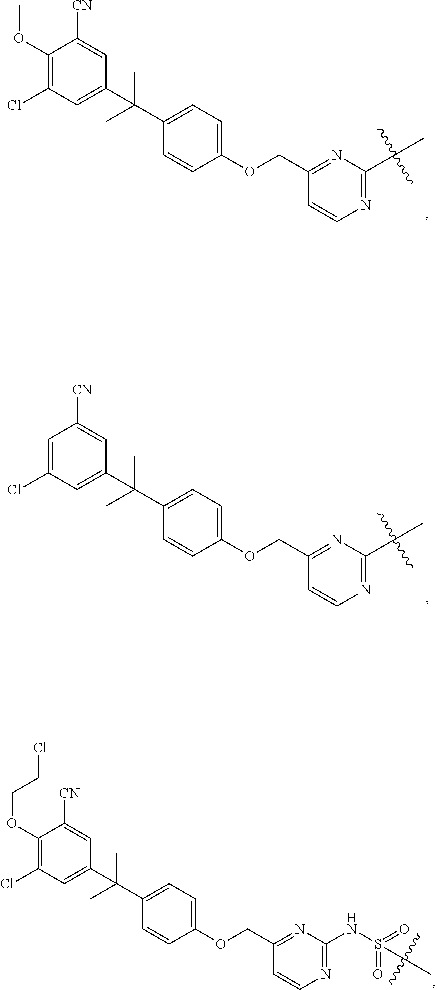 [SMILES: *c1nccc(COc2ccc(C(C)(C)c3cc(Cl)c(OC)c(C#N)c3)cc2)n1 ; *c1nccc(COc2ccc(C(C)(C)c3cc(Cl)cc(C#N)c3)cc2)n1 ; *S(=O)(=O)Nc1nccc(COc2ccc(C(C)(C)c3cc(Cl)c(OCCCl)c(C#N)c3)cc2)n1]

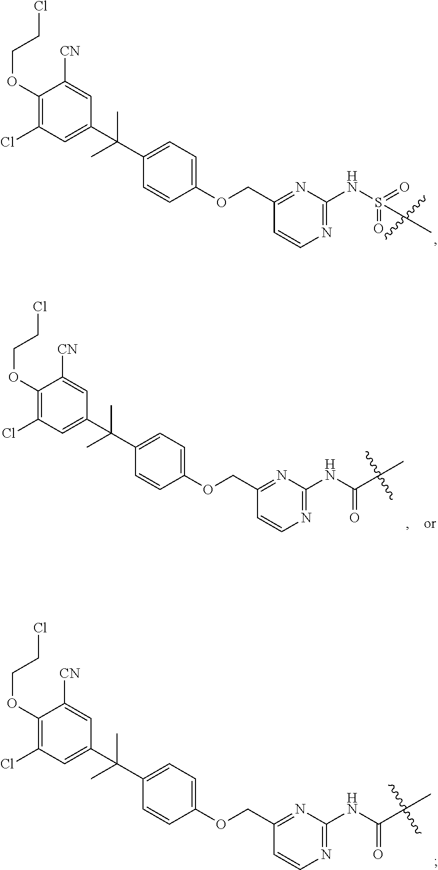 [SMILES: *S(=O)(=O)Nc1nccc(COc2ccc(C(C)(C)c3cc(Cl)c(OCCCl)c(C#N)c3)cc2)n1 ; *C(=O)Nc1nccc(COc2ccc(C(C)(C)c3cc(Cl)c(OCCCl)c(C#N)c3)cc2)n1 ; *C(=O)Nc1nccc(COc2ccc(C(C)(C)c3cc(Cl)c(OCCCl)c(C#N)c3)cc2)n1]

in some embodiments, B is selected from

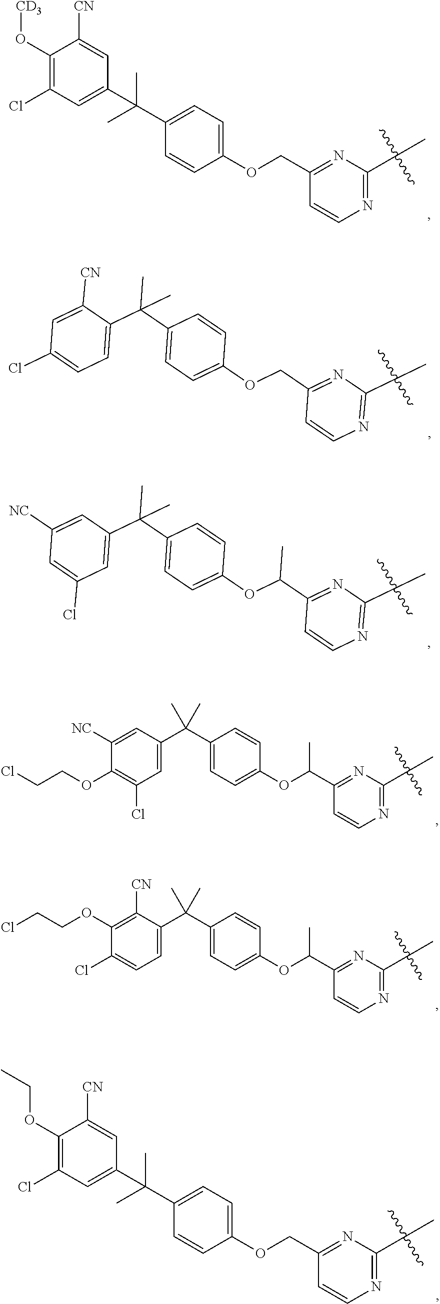 [SMILES: *c1nccc(COc2ccc(C(C)(C)c3cc(Cl)c(OC([2H])([2H])[2H])c(C#N)c3)cc2)n1 ; *c1nccc(COc2ccc(C(C)(C)c3ccc(Cl)cc3C#N)cc2)n1 ; *c1nccc(C(C)Oc2ccc(C(C)(C)c3cc(Cl)cc(C#N)c3)cc2)n1 ; *c1nccc(C(C)Oc2ccc(C(C)(C)c3cc(Cl)c(OCCCl)c(C#N)c3)cc2)n1 ; *c1nccc(C(C)Oc2ccc(C(C)(C)c3ccc(Cl)c(OCCCl)c3C#N)cc2)n1 ; *c1nccc(COc2ccc(C(C)(C)c3cc(Cl)c(OCC)c(C#N)c3)cc2)n1]

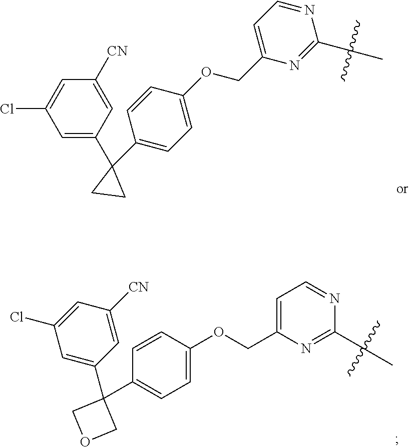 [SMILES: *c1nccc(COc2ccc(C3(c4cc(Cl)cc(C#N)c4)CC3)cc2)n1 ; *c1nccc(COc2ccc(C3(c4cc(Cl)cc(C#N)c4)COC3)cc2)n1]

in some embodiments, B is selected from one of the groups in Table B-1, wherein the right side thereof is directly linked to L;
in some embodiments, B is selected from one of the groups in Table B-2, wherein the right side thereof is directly linked to L;
in some embodiments, K is selected from

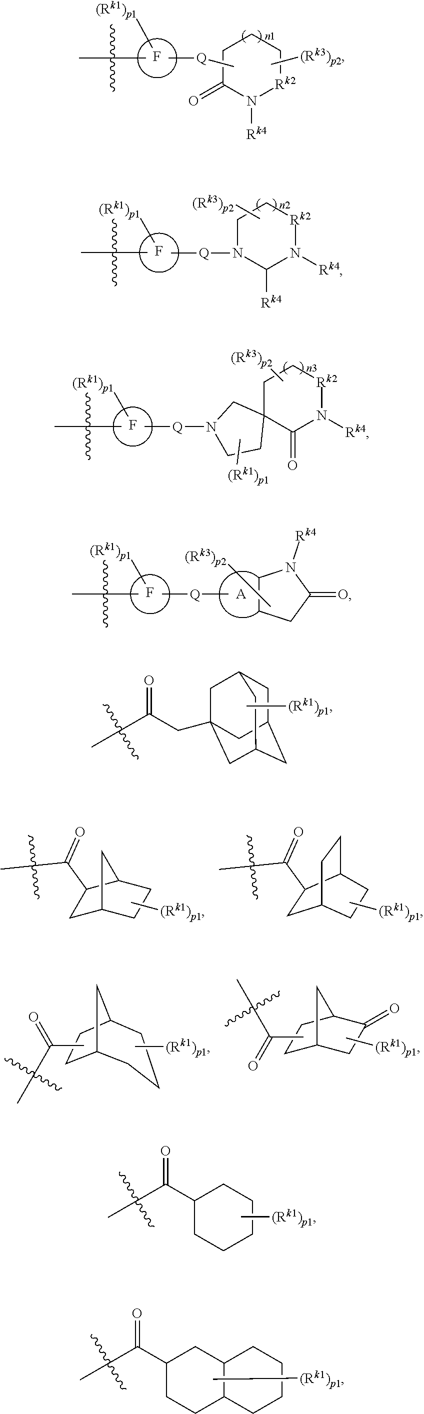

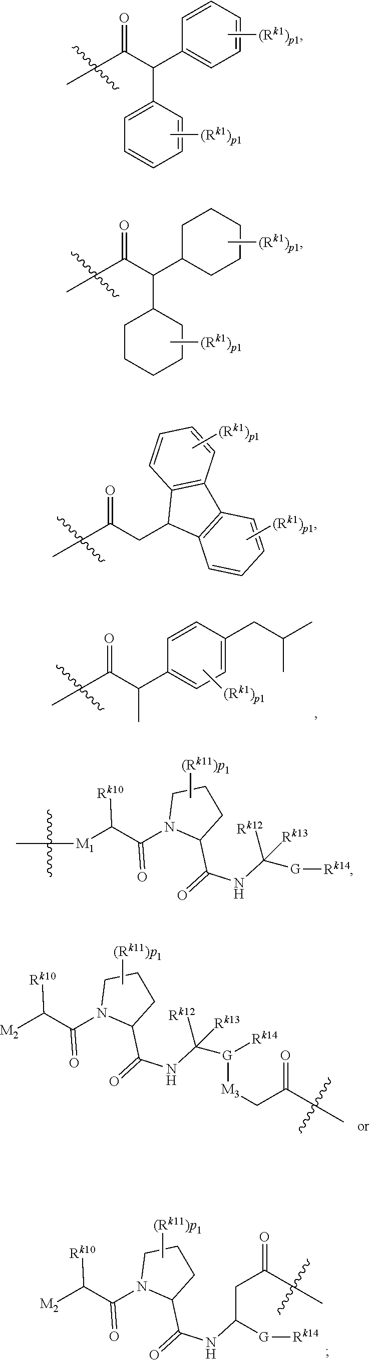

in some embodiments, K is selected from

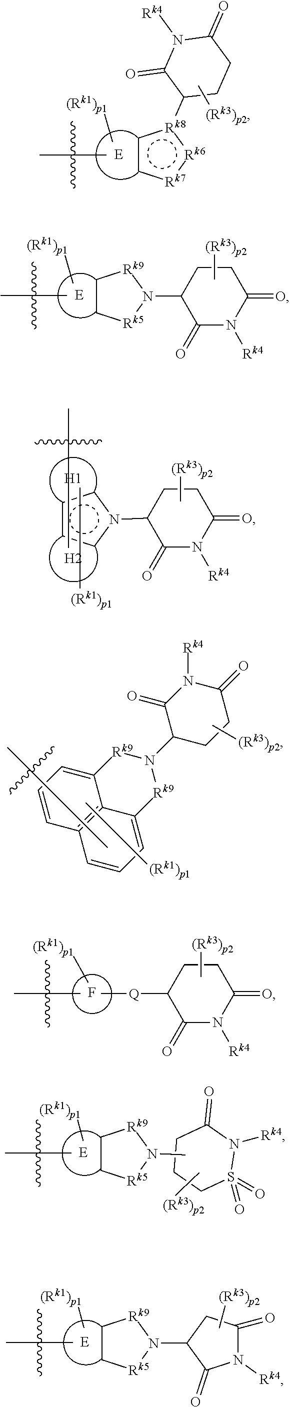

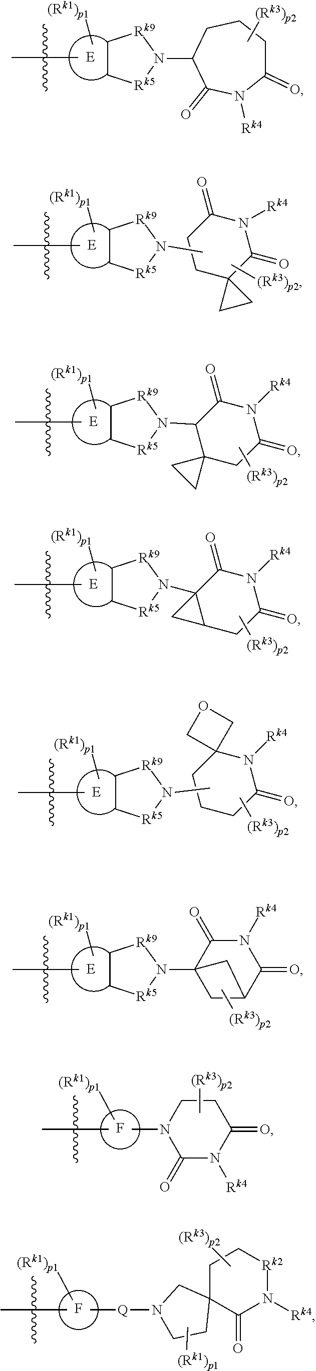

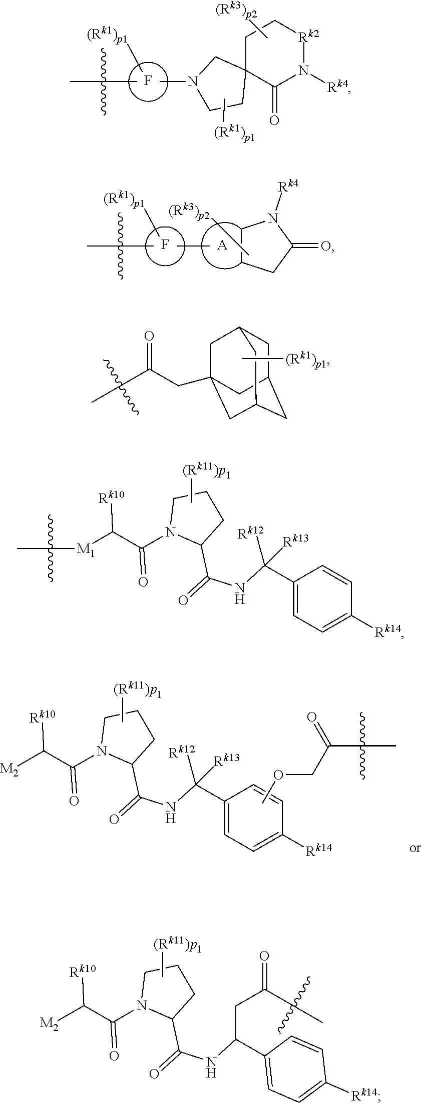

in some embodiments, K is selected from

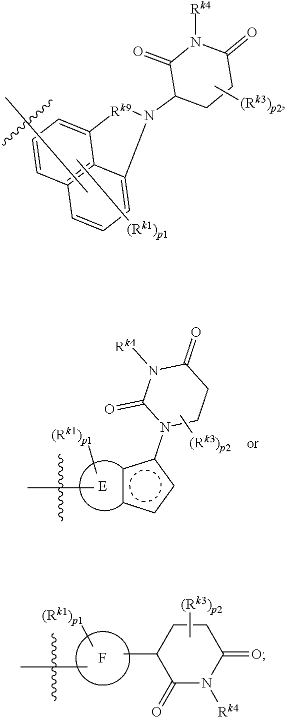

in some embodiments, K is selected from

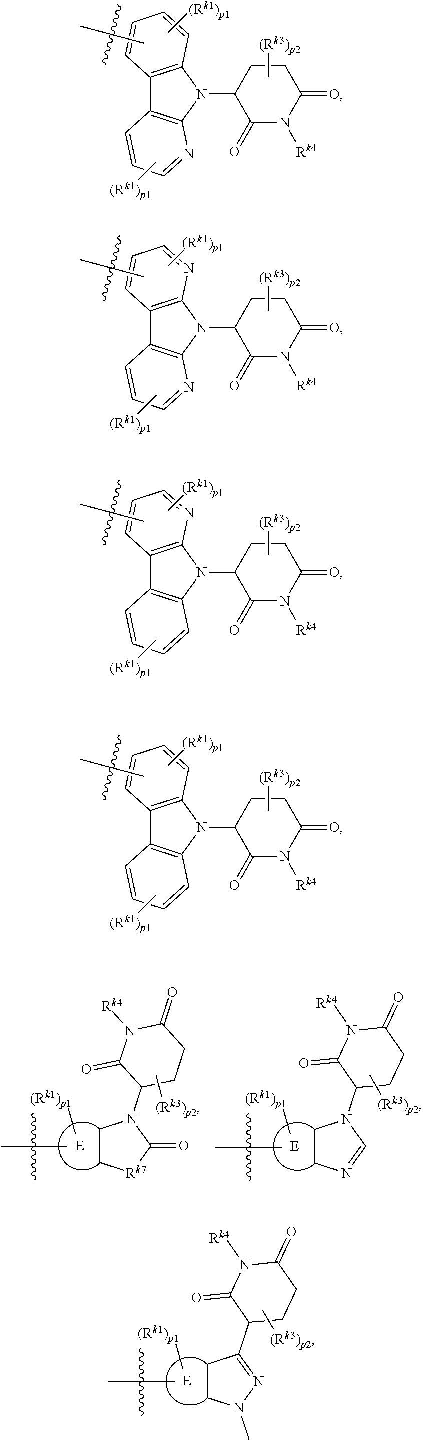

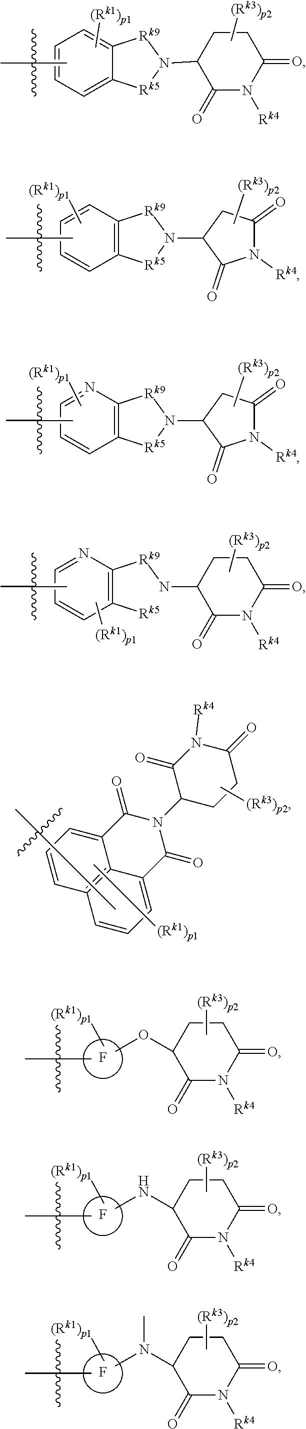

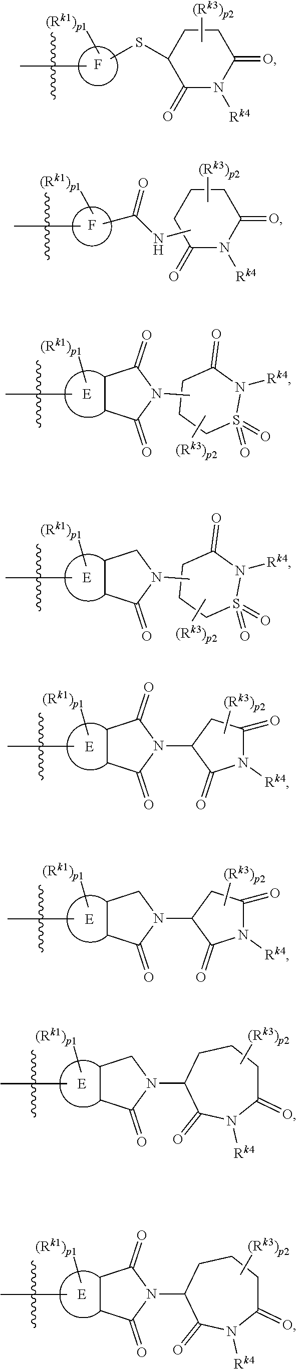

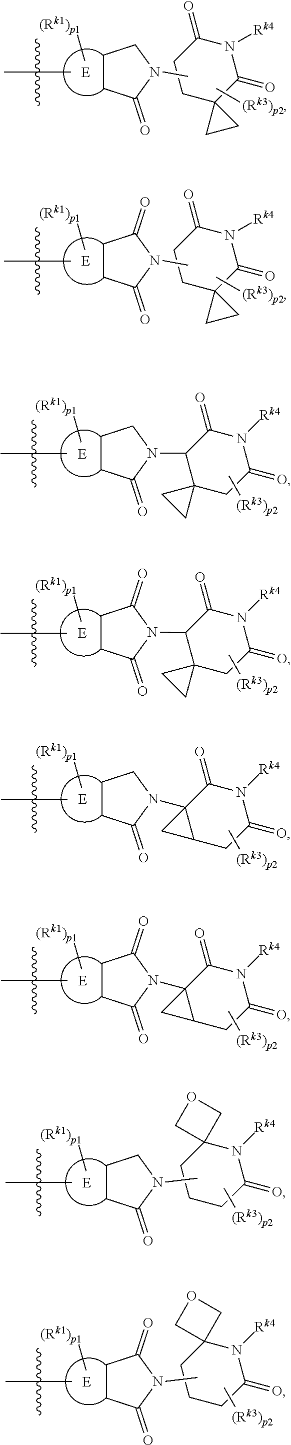

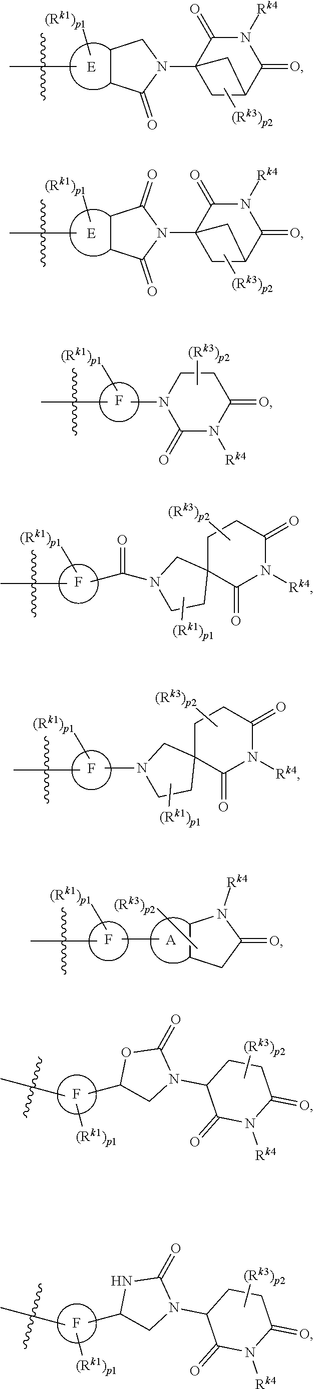

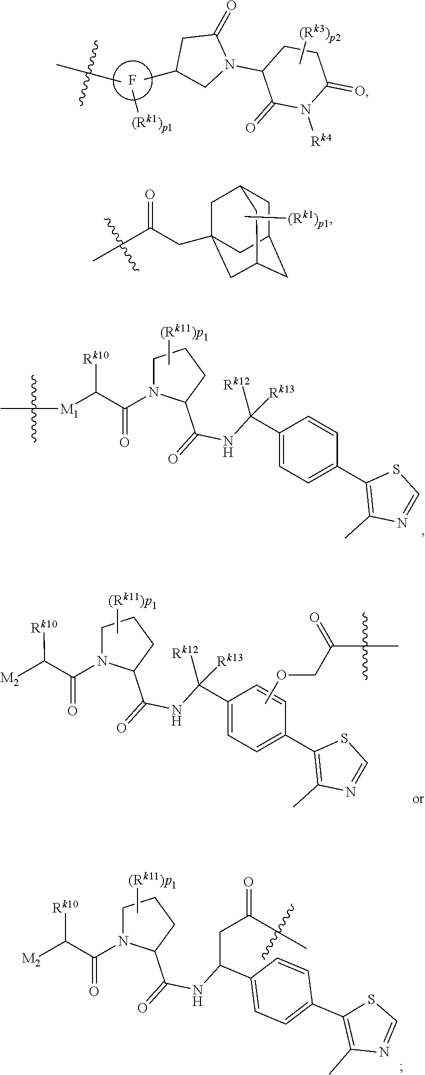

in some embodiments, K is selected from

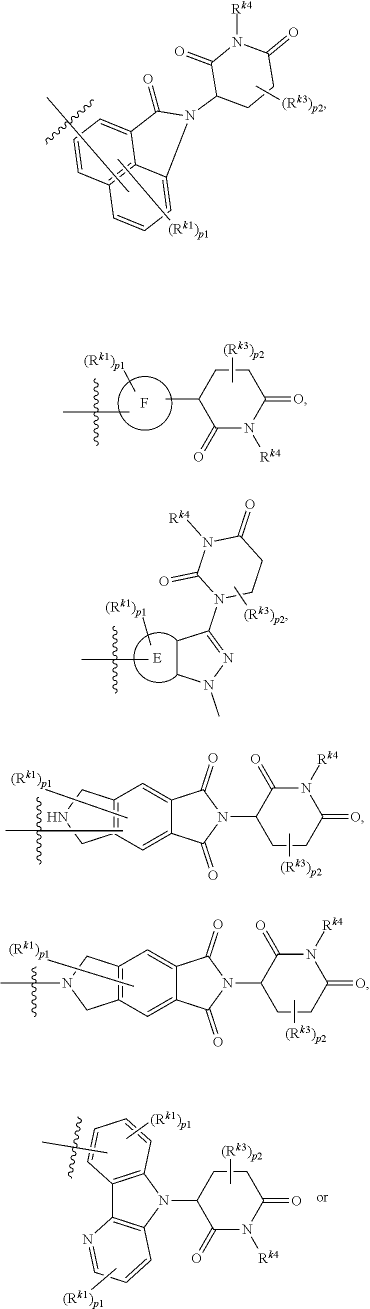

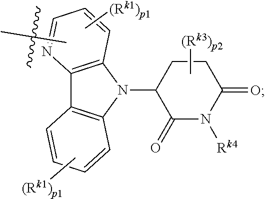

in some embodiments, each Q is independently selected from a bond, —O—, —S—, —CH₂—, —NR^{q}—, —CO—, —NR^{q}CO—, —CONR^{q} or 3- to 12-membered heterocyclyl, wherein the heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
in some embodiments, each Q is independently selected from —O—, —S—, —CH₂—, —NR^{q}—, —CO—, —NR^{q}CO, —CONR^{q}— or 4- to 7-membered heterocyclyl, wherein the heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
in some embodiments, R⁴ is selected from H or C₁₋₆ alkyl;
in some embodiments, R^{q} is selected from H or C₁₋₄ alkyl;
in some embodiments, R^{q} is selected from H, methyl, or ethyl;
in some embodiments, each E is independently selected from C₃₋₁₀ carbocyclyl, C₆₋₁₀ aryl, 3- to 12-membered heterocyclyl or 5- to 12-membered heteroaryl, wherein the heterocycle or heteroaryl contains 1 to 4 (for example, 1, 2, 3 or 4) heteroatoms selected from O, S, or N;
in some embodiments, each E is independently selected from C₃₋₁₀ carbocyclyl, C₆₋₁₀ aryl, 3- to 10-membered heterocyclyl or 5- to 10-membered heteroaryl, wherein the heterocycle or heteroaryl contains 1 to 4 (for example, 1, 2, 3 or 4) heteroatoms selected from O, S, or N;
in some embodiments, each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 (for example, 1, 2, 3 or 4) heteroatoms selected from O, S, or N;
in some embodiments, each E is independently selected from phenyl, pyridyl, pyridazinyl, pyrazinyl, pyrimidinyl, pyrrolyl, pyrazolyl, imidazolyl, thiazolyl, furyl, thienyl, oxazolyl, indolinyl, isoindolinyl, 1,2,3,4-tetrahydroquinolinyl or 1,2,3,4-tetrahydroisoquinolinyl;
in some embodiments, each E is independently selected from phenyl, pyridyl, pyridazinyl, pyrazinyl, pyrimidinyl, pyrrolyl, pyrazolyl, imidazoyl, thiazolyl, furyl, thienyl or oxazolyl;
in some embodiments, each E is independently selected from phenyl, pyridyl, pyridazinyl, pyrazinyl, or pyrimidinyl;
in some embodiments, each E is independently selected from a benzene ring or a pyridine ring;
in some embodiments, each A, H1 or H2 is independently selected from C₃₋₈ carbocycle, a benzene ring, 4- to 7-membered heterocycle or 5- to 6-membered heteroaryl, wherein the heterocycle or heteroaryl contains 1 to 4 heteroatoms selected from O, S, or N;
in some embodiments, each A, H1 or H2 is independently selected from phenyl, pyridyl, pyridazinyl, pyrazinyl, pyrimidinyl, pyrrolyl, pyrazolyl, imidazoyl, thiazolyl, furyl, thienyl or oxazolyl; in some embodiments, each A, H1 or H2 is independently selected from phenyl or pyridyl;
in some embodiments, each F is independently selected from C₃₋₂₀ carbocyclyl, C₆₋₂₀ aryl, 3- to 20-membered heterocyclyl or 5- to 20-membered heteroaryl, wherein the heterocyclyl or heteroaryl contains 1 to 4 (for example, 1, 2, 3 or 4) heteroatoms selected from O, S, or N;
in some embodiments, each F is independently selected from 3- to 7-membered monocycloalkyl, 4- to 10-membered fused cycloalkyl, 5- to 12-membered spiro cycloalkyl, 5- to 10-membered bridged cycloalkyl, 4- to 7-membered mono-heterocyclic ring, 4- to 10-membered fused heterocyclic ring, 5- to 12-membered spiro-heterocyclic ring, 5- to 10-membered bridged-heterocyclic ring, C₆₋₁₄ aryl or 5- to 10-membered heteroaryl, wherein the mono-heterocyclic ring, fused heterocyclic ring, spiro-heterocyclic ring, bridged-heterocyclic ring or heteroaryl contains 1 to 4 (for example, 1, 2, 3 or 4) heteroatoms selected from O, S, or N;
in some embodiments, each F is independently selected from cyclopropyl, cyclobutyl, cyclopentyl, cyclohexyl, phenyl, naphthyl, anthryl, phenanthrenyl, azetidinyl, azacyclopentyl, piperidyl, morpholinyl, pyridyl, pyrimidinyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidinyl, benzopyridazinyl, pyrrolopyrrolyl, pyrrolopyridinyl, pyrrolopyrimidinyl, pyrrolopyridazinyl, pyrrolopyrazinyl, imidazopyrimidinyl, imidazopyridinyl, imidazopyrazinyl, imidazopyridazinyl, pyrazolopyridinyl, pyrazolopyrimidinyl, pyrazolopyridazinyl, pyrazolopyrazinyl, pyrimidopyridyl, pyrimidopyrazinyl, pyrimidopyridazinyl, pyrimidopyrimidinyl, pyridopyridyl, pyridopyrazinyl, pyridopyridazinyl, pyridazopyridazinyl, pyridazopyrazinyl or pyrazinopyrazinyl;
in some embodiments, each R^{k2} is independently selected from a bond, —CO—, —SO₂—, —SO— or —C(R^{k3})₂—;
in some embodiments, each R^{k2} is independently selected from —CO—, —SO₂— or —C(R^{k3})₂—;
in some embodiments, each R^{k1} is independently selected from H, F, Cl, Br, I, OH, ═O, NH₂, CN, COOH, CONH₂, C₁₋₆ alkyl or C₁₋₆ alkoxy, wherein the alkyl or alkoxy is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy;
in some embodiments, each R^{k3} is independently selected from H, F, Cl, Br, I, OH, ═O, NH₂, CN, COOH, CONH₂, C₁₋₆ alkyl, C₁₋₆ alkoxy, C₃₋₈ cycloalkyl or 3- to 8-membered heterocyclyl, wherein the alkyl, alkoxy, cycloalkyl or heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
in some embodiments, R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 substituents selected from H, F, Cl, Br, I, OH, or NH₂;
in some embodiments, R^{k1} and R^{k3} are each independently selected from H, F, Cl, Br, I, OH, ═O, NH₂, CF₃, CN, COOH, CONH₂, methyl, ethyl, isopropyl, methoxy, ethoxy or isopropoxy, wherein the methyl, ethyl, isopropyl, methoxy, ethoxy or isopropoxy is optionally further substituted with 0 to 4 substituents selected from H, F, Cl, Br, I, OH, or NH₂;
in some embodiments, two R^{k3} together with the carbon atoms or ring backbones to which they are directly attached form 3- to 8-membered carbocycle or 3- to 8-membered heterocycle, wherein the carbocycle or heterocycle is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocycle contains 1 to 4 (for example, 1, 2, 3 or 4) heteroatoms selected from O, S, or N;
in some embodiments, two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocycle contains 1 to 4 (for example, 1, 2, 3 or 4) heteroatoms selected from O, S, or N;
in some embodiments, each R^{k4} is independently selected from H, OH, NH₂, CN, CONH₂, C₁₋₆ alkyl, C₃₋₈ cycloalkyl or 3- to 8-membered heterocyclyl, wherein the alkyl, cycloalkyl or heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
in some embodiments, each R^{k4} is independently selected from H, OH, NH₂, CF₃, CN, or C₁₋₄ alkyl;
in some embodiments, each R^{k5} is independently selected from CO, CH₂, SO₂ or

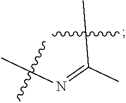 [SMILES: */N=C(\*)C]

in some embodiments, each R^{k6} is independently selected from CO, CH, SO, SO₂, CH₂ or N;
in some embodiments, each R^{k7} is independently selected from CO, CH, N, CH₂, O, S, N(CH₃) or NH;
in some embodiments, each R^{k7} is independently selected from CH₂, O, N(CH₃) or NH;
in some embodiments, each R^{k8} is independently selected from C, N or CH;
in some embodiments, each R^{k9} is independently selected from CO, SO₂ or CH₂;
in some embodiments, M₁ is selected from a bond, —CH₂—C(═O)NH— or —C(═O)CH₂NH—;
in some embodiments, M₂ is selected from —NHC(═O)—C₁₋₆ alkyl, —NHC(═O)—C₃₋₆ cycloalkyl or 4- to 10-membered heterocyclyl, wherein the alkyl, cycloalkyl or heterocyclyl is optionally further substituted with 0 to 4 (for example, 0, 1, 2, 3 or 4) substituents selected from H, F, Cl, Br, I, ═O, OH, NH₂, C₁₋₄ alkyl or C₁₋₄ alkoxy, and the heterocyclyl contains 1 to 4 (for example, 1, 2, 3 or 4) heteroatoms selected from 0, S, or N;
in some embodiments, M₃ is selected from —NH— or —O—;
in some embodiments, R^{k10} is selected from C₁₋₆ alkyl, wherein the alkyl is optionally further substituted with 0 to 4 (for example, 0, 1, 2, 3 or 4) substituents selected from H, F, Cl, Br, I, ═O, OH, C₁₋₆ alkyl or C₃₋₆ cycloalkyl;
in some embodiments, G is selected from 6- to 10-membered aryl or 5- to 10-membered heteroaryl, wherein the aryl or heteroaryl is optionally further substituted with 0 to 4 (for example, 0, 1, 2, 3 or 4) substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 (for example, 1, 2, 3 or 4) heteroatoms selected from N, O, or S;
in some embodiments, each R^{k11} is independently selected from H, F, Cl, Br, I, ═O, OH, SH, C₁₋₆ alkyl, C₁₋₆ alkoxy or C₁₋₆ alkylthio or —O—C(═O)—C₁₋₆ alkyl, wherein the alkyl, alkoxy or alkylthio is optionally further substituted with 0 to 4 (for example, 0, 1, 2, 3 or 4) substituents selected from H, F, Cl, Br, I, OH, C₁₋₄ alkyl or C₁₋₄ alkoxy;
in some embodiments, R^{k12} and R^{k13} are each independently selected from H, C₁₋₆ alkyl or C₃₋₆ cycloalkyl, wherein the alkyl or cycloalkyl is optionally further substituted with 0 to 4 (for example, 0, 1, 2, 3 or 4) substituents selected from H, F, Cl, Br, I, ═O, OH, NH₂, C₁₋₄ alkyl or C₁₋₄ alkoxy;
in some embodiments, R^{k14} is selected from 5- to 6-membered heteroaryl, wherein the heteroaryl is optionally further substituted with 0 to 4 (for example, 0, 1, 2, 3 or 4) substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 (for example, 1, 2, 3 or 4) heteroatoms selected from N, O, or S;
in some embodiments, K is selected from

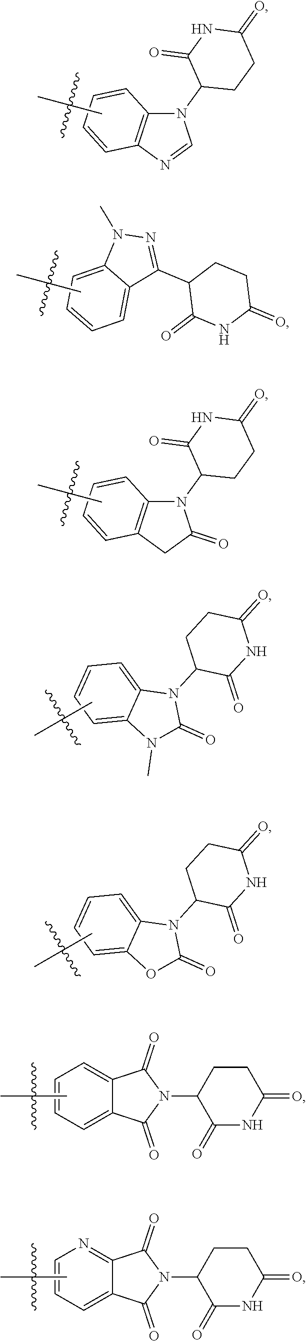

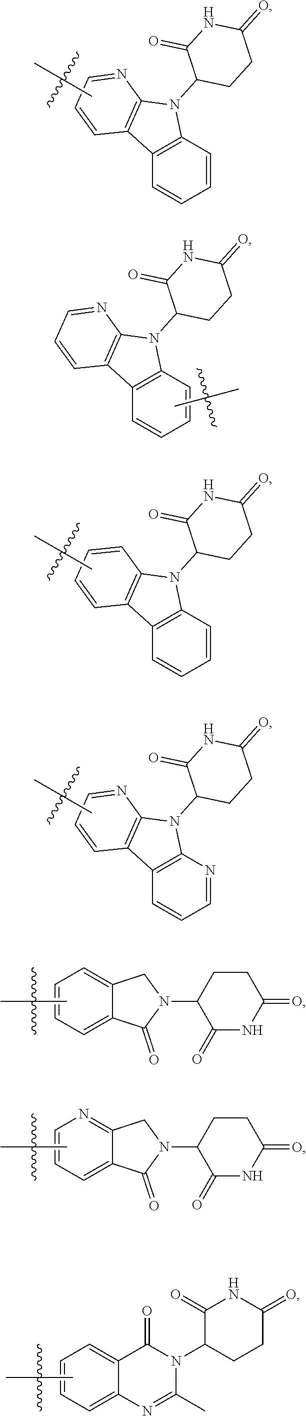

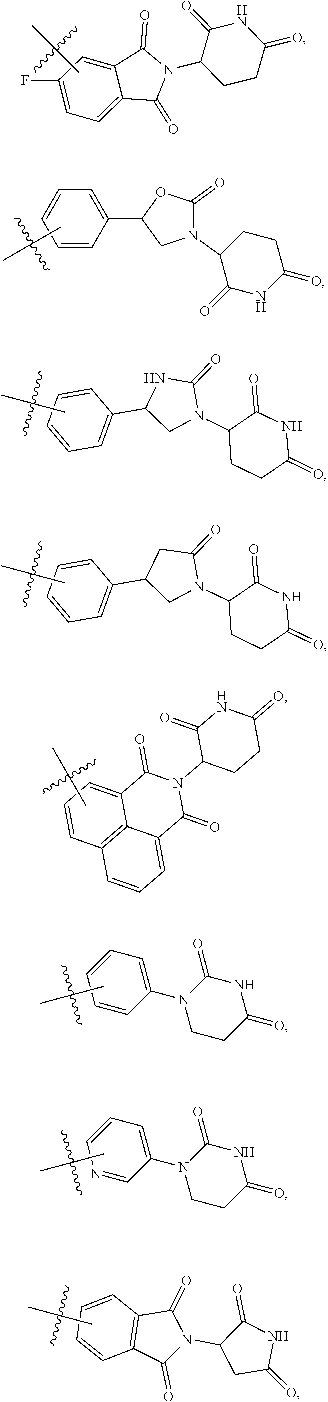

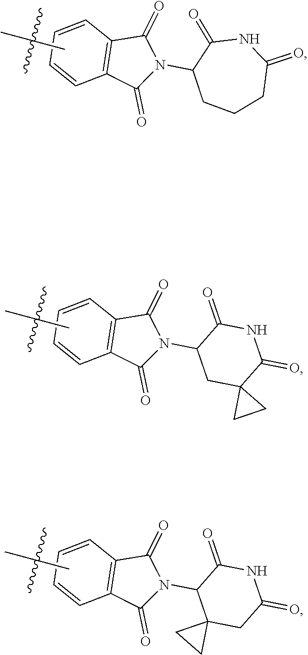

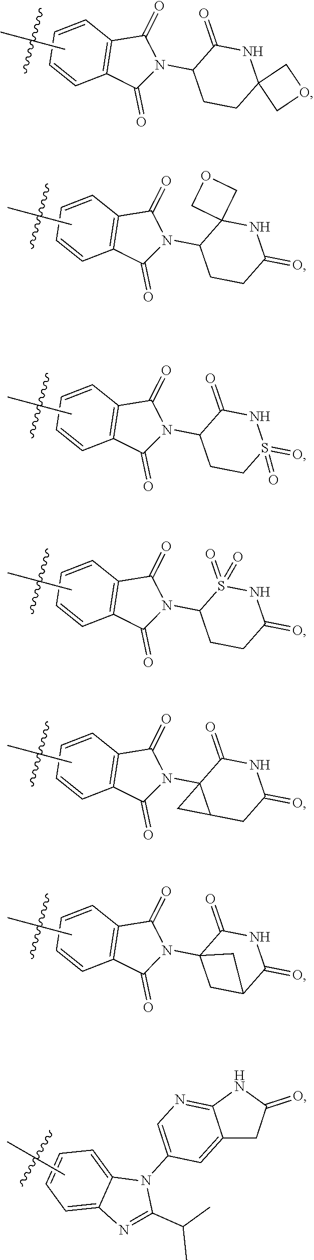

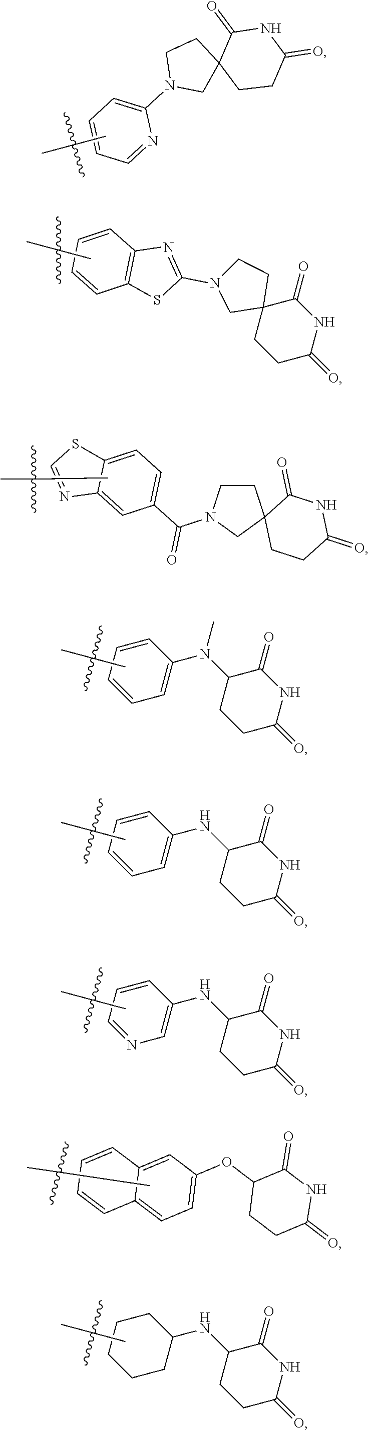

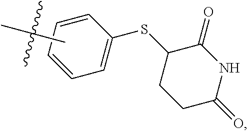

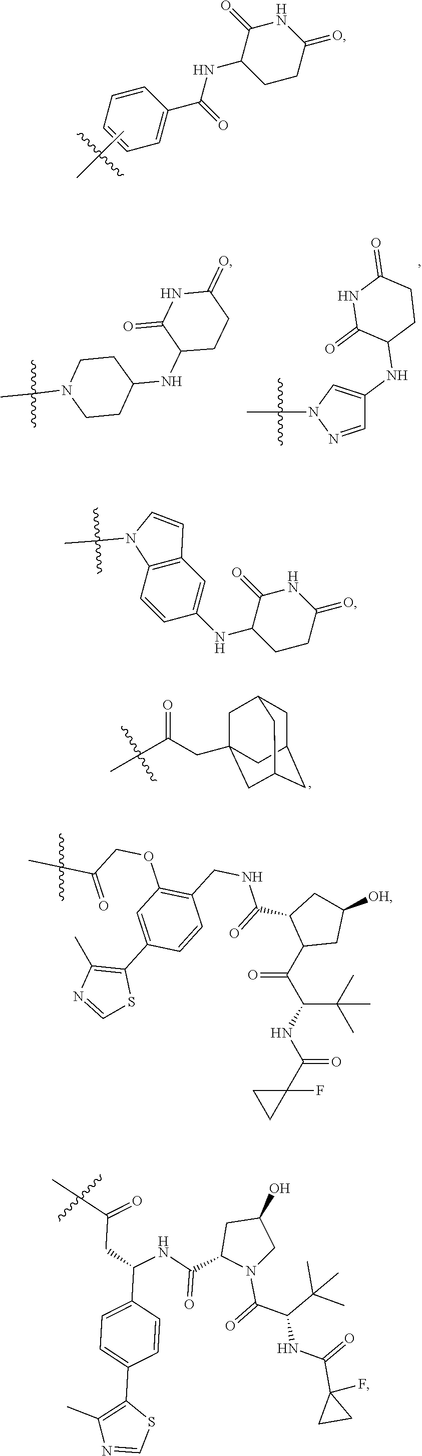

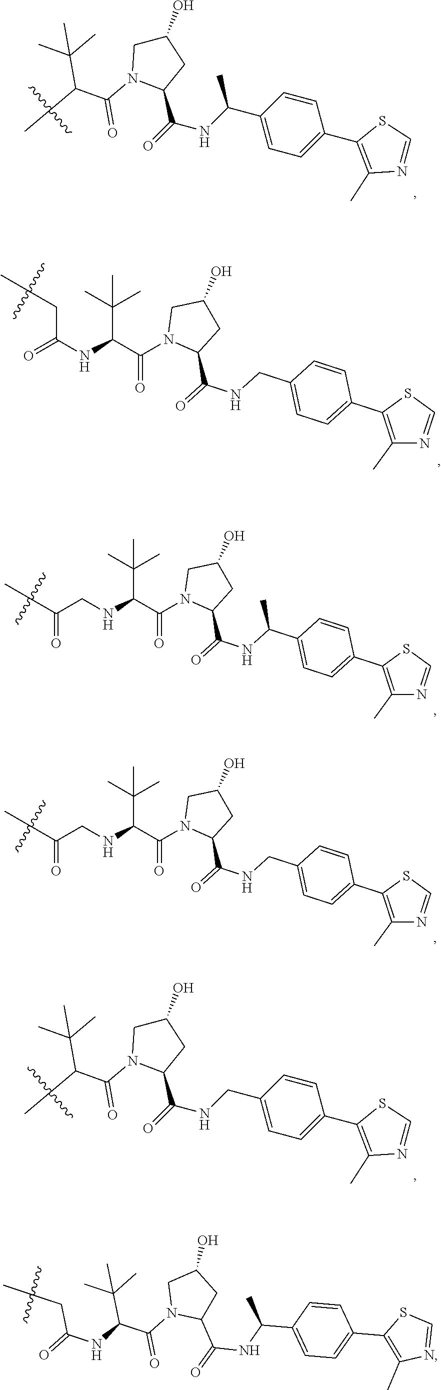 [SMILES: *C(C(=O)N1C[C@H](O)C[C@H]1C(=O)N[C@@H](C)c1ccc(-c2scnc2C)cc1)C(C)(C)C ; *CC(=O)N[C@H](C(=O)N1C[C@H](O)C[C@H]1C(=O)NCc1ccc(-c2scnc2C)cc1)C(C)(C)C ; *C(=O)CN[C@H](C(=O)N1C[C@H](O)C[C@H]1C(=O)N[C@@H](C)c1ccc(-c2scnc2C)cc1)C(C)(C)C ; *C(=O)CN[C@H](C(=O)N1C[C@H](O)C[C@H]1C(=O)NCc1ccc(-c2scnc2C)cc1)C(C)(C)C ; *C(C(=O)N1C[C@H](O)C[C@H]1C(=O)NCc1ccc(-c2scnc2C)cc1)C(C)(C)C ; *CC(=O)N[C@H](C(=O)N1C[C@H](O)CC1C(=O)N[C@@H](C)c1ccc(-c2scnc2C)cc1)C(C)(C)C]

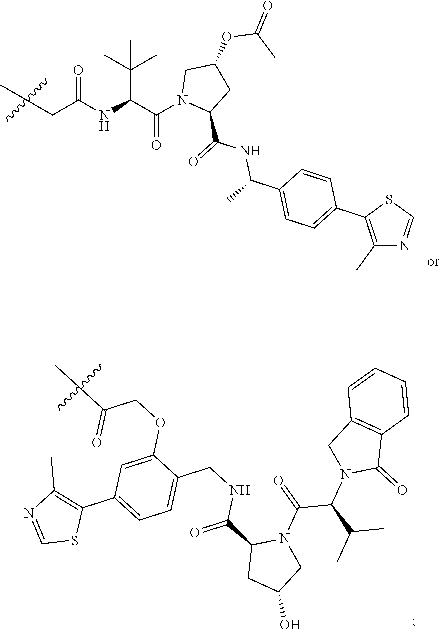 [SMILES: *CC(=O)N[C@H](C(=O)N1C[C@H](OC(C)=O)C[C@H]1C(=O)N[C@@H](C)c1ccc(-c2scnc2C)cc1)C(C)(C)C ; *C(=O)COc1cc(-c2scnc2C)ccc1CNC(=O)[C@@H]1C[C@@H](O)CN1C(=O)[C@H](C(C)C)N1Cc2ccccc2C1=O]

in some embodiments, K is selected from

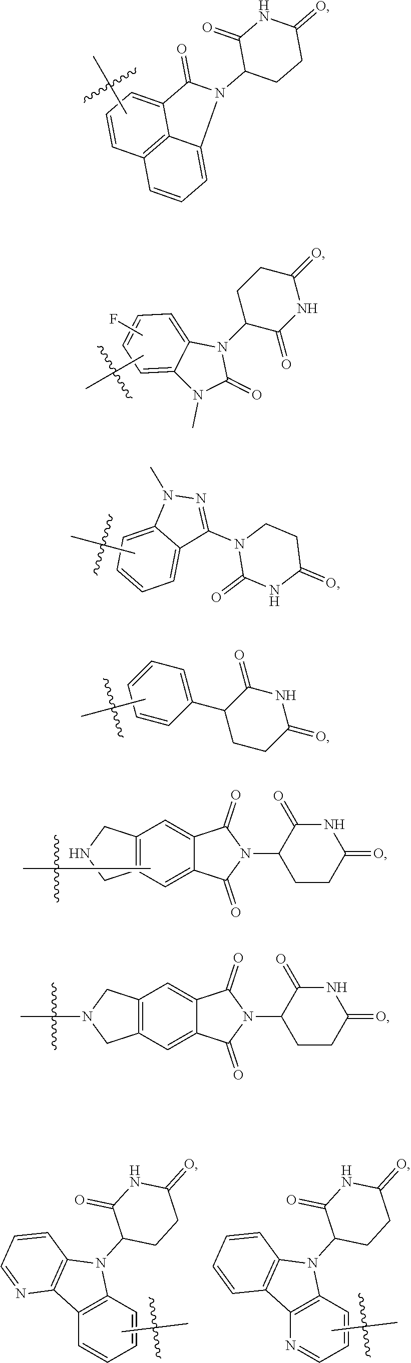

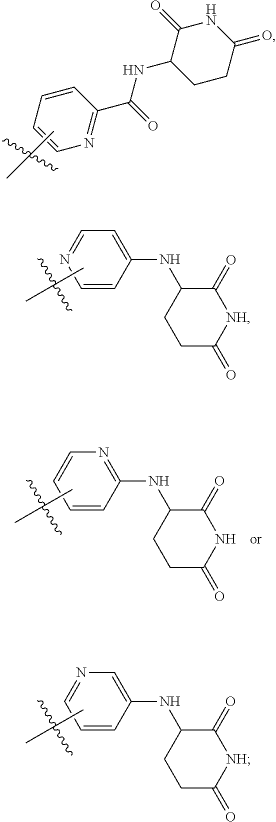

in some embodiments, K is selected from

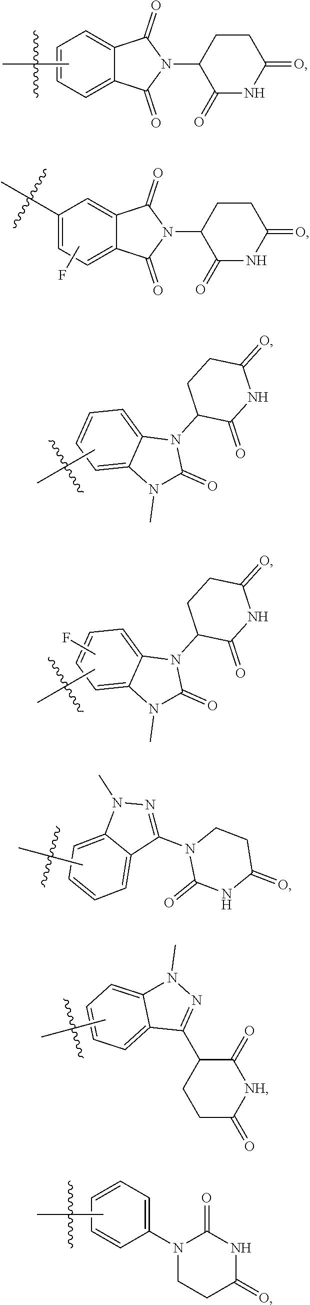

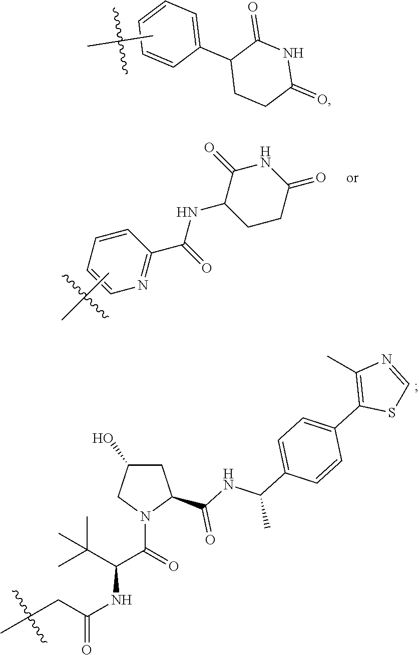

in some embodiments, K is selected from,

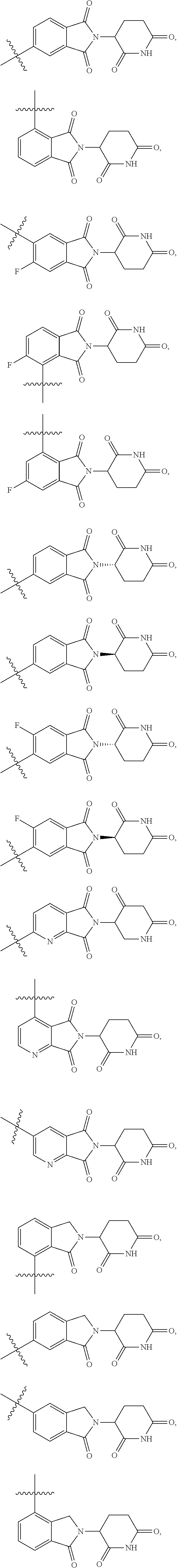

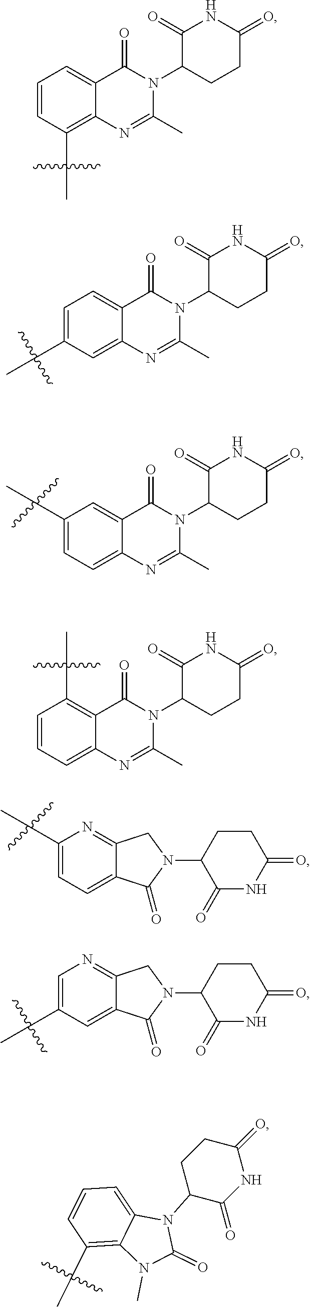

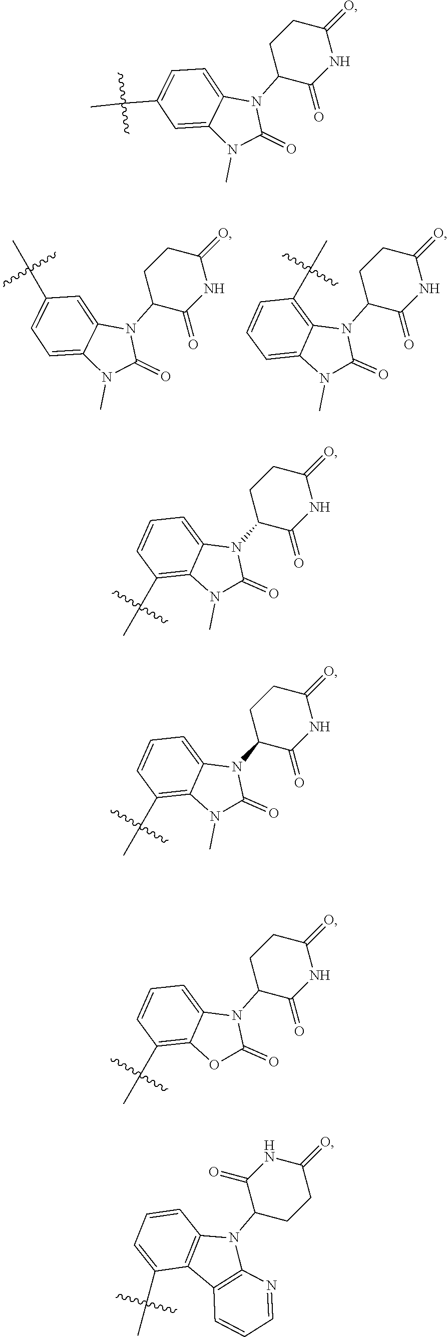

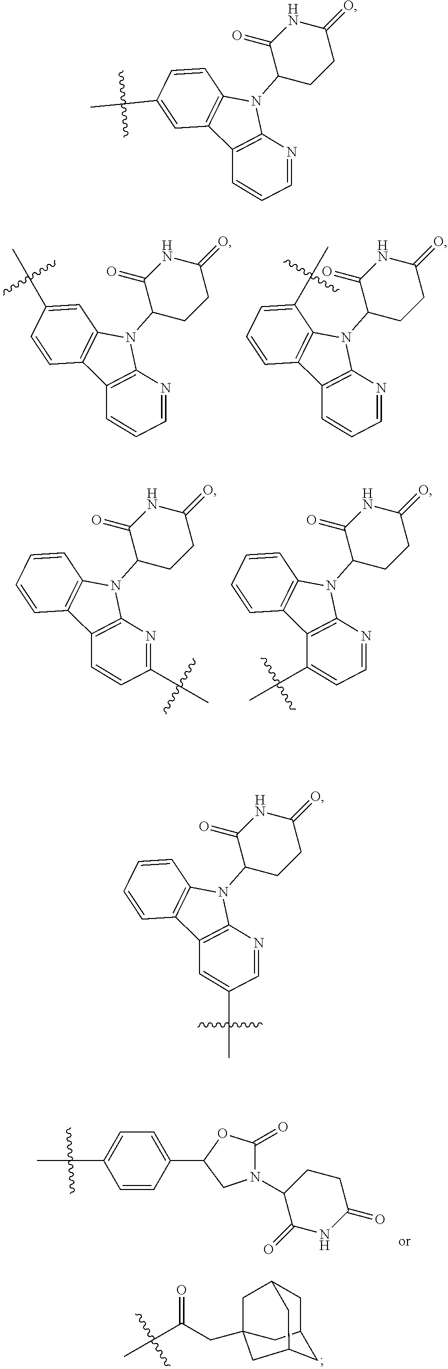

in some embodiments, K is selected from

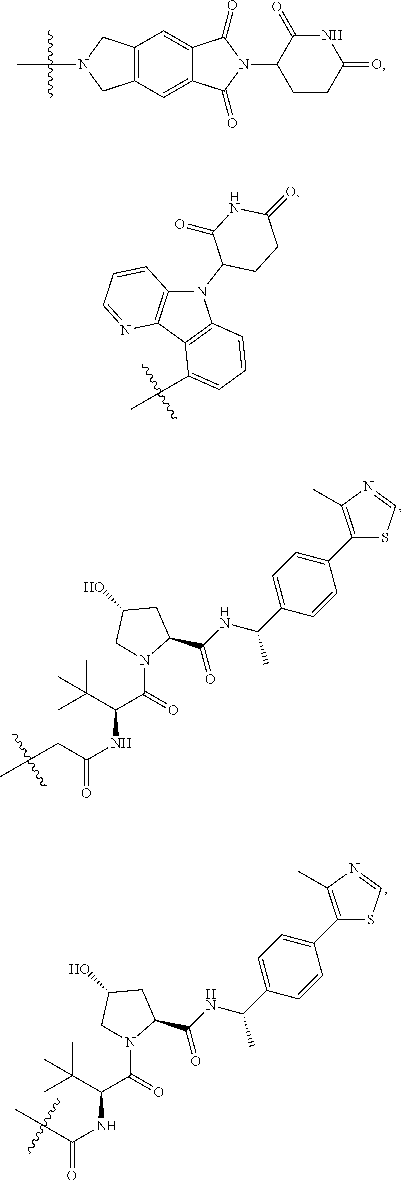 [SMILES: *N1Cc2cc3c(cc2C1)C(=O)N(C1CCC(=O)NC1=O)C3=O ; *c1cccc2c1c1ncccc1n2C1CCC(=O)NC1=O ; *CC(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

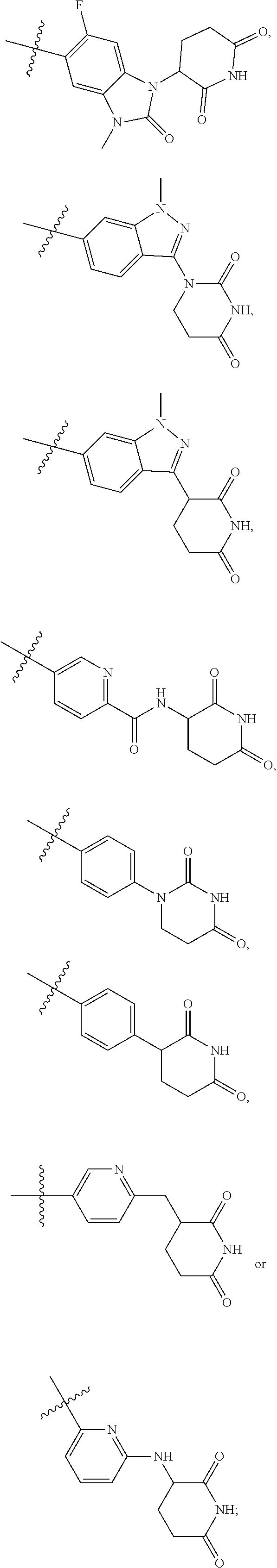

in some embodiments, K is selected from

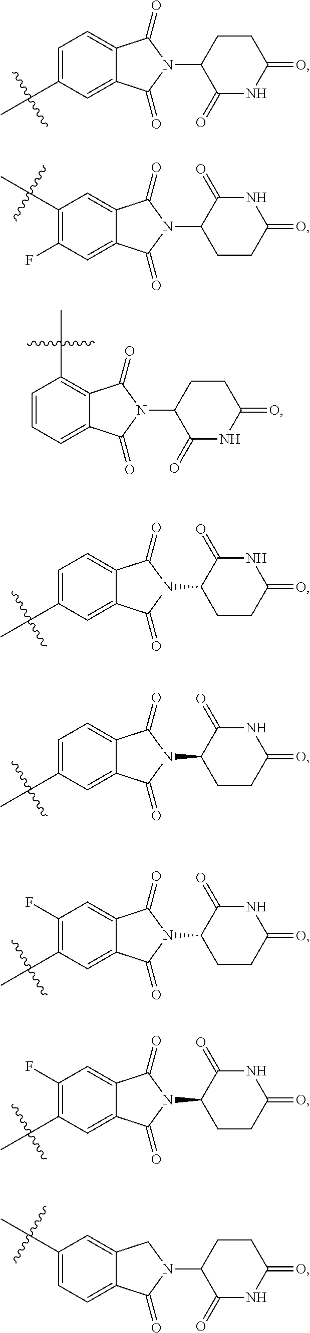

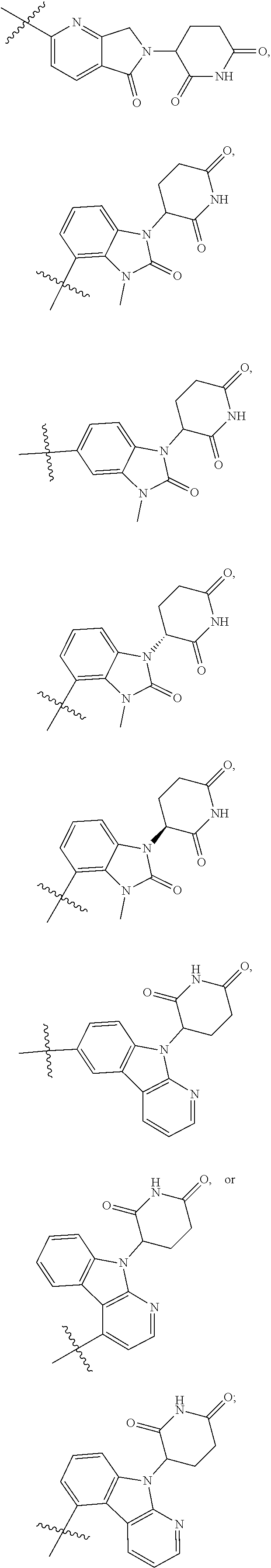

in some embodiments, K is selected from one of the groups in Table K-1, wherein the left side thereof is linked to L;
in some embodiments, each q is independently selected from 0, 1, 2, 3, 4, 5 or 6;
in some embodiments, n, q, r, and t are each independently selected from 0, 1, 2, 3 or 4;
in some embodiments, m1, m2, m3, n1, n2, and n3 are each independently selected from 0, 1, 2 or 3;
in some embodiments, r is selected from 1 or 2;
in some embodiments, each t is independently selected from 0, 1 or 2;
in some embodiments, each q is independently selected from 0, 1 or 2;
in some embodiments, each p1 or p2 is independently selected from 0, 1, 2, 3, 4 or 5;
in some embodiments, each p1 or p2 is independently selected from 0, 1 or 2;
in some embodiments, when B is selected from

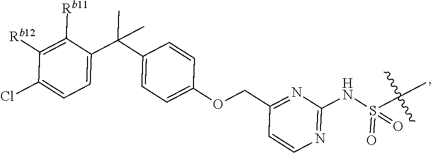

at least one of Cy1, Cy2, Cy3, and Cy4 cannot be a bond, and when three of them are selected from a bond, the remaining one cannot be selected from triazolyl;
in some embodiments, when B is selected from

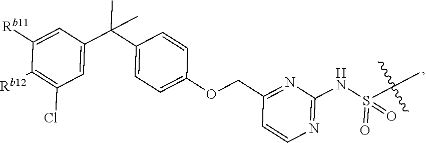

at least one of Cy1, Cy2, Cy3, and Cy4 cannot be a bond, and when three of them are selected from a bond, the remaining one cannot be selected from triazolyl.

As a first embodiment of the present invention, the above-mentioned compound as represented by general formula (I) or the stereoisomer, deuterated compound, solvate, prodrug, metabolite, pharmaceutically acceptable salt or co-crystal thereof, wherein
L is selected from a bond or C₁₋₅₀ hydrocarbyl, wherein 0 to 20 methylene units in the hydrocarbyl are replaced by -Ak- and -Cy-;
each -Ak- is independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}—, —(C≡C)_{q}—, —CH═CH—, —Si(R^{L})₂—, —Si(OH)(R^{L})—, —Si(OH)₂—, —P(═O)(OR^{L})—, —P(═O)(R^{L})—, —S(═O)—, —S(═O)₂— or a bond;
each q is independently selected from 0, 1, 2, 3, 4, 5 or 6;
each R^{L} is independently selected from H, C₁₋₆ alkyl, 3- to 7-membered heterocyclyl, 3- to 7-membered cycloalkyl, phenyl or 5- to 6-membered heteroaryl;
each -Cy- is independently selected from a bond, 4- to 8-membered mono-heterocyclic ring, 4- to 10-membered fused heterocyclic ring, 5- to 12-membered spiro-heterocyclic ring, 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 (for example, 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 (for example, 1, 2, 3 or 4) heteroatoms selected from O, S or N;
B is selected from

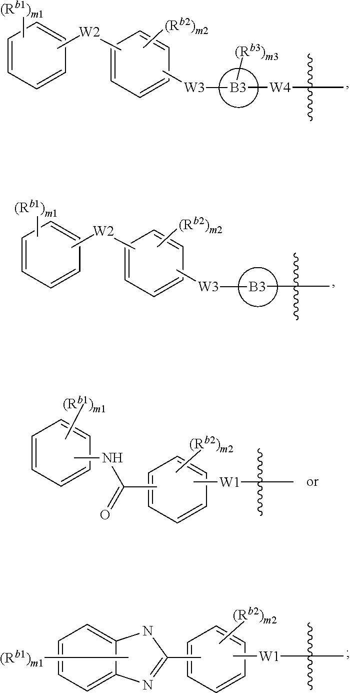

each W1 is independently selected from a bond, —O—, —S—, —NR^{w1}—, —NR^{w1}C(═O)—, —NR^{w1}S(═O)₂—, or —NR^{w1}S(═O)₂NR^{w1}—, wherein the left side thereof is directly linked to phenyl;
each W2 is independently selected from —NR^{w1}— or —(CR^{w2}R^{w3})ᵣ—, wherein the left side thereof is directly linked to phenyl;
each W3 is independently selected from —O(CR^{w2}R^{w3})ₜ—, —S(CR^{w2}R^{w3})ₜ—, or —NR^{w1}(CR^{w2}R^{w3})ₜ—, wherein the left side thereof is directly linked to phenyl;
W4 is selected from —O—, —S—, —NR^{w1}—, —NR^{w1}C(═O)—, —NR^{w1}S(═O)₂—, or —NR^{w1}S(═O)₂NR^{w1}—, wherein the left side thereof is directly linked to phenyl;
B3 is selected from 5- to 6-membered heteroaryl, wherein the heteroaryl contains 1 to 4 (for example, 1, 2, 3 or 4) heteroatoms selected from O, S, or N;
each R^{w1} is independently selected from H, C₁₋₄ alkyl, or C₃₋₆ cycloalkyl;
each R^{w2} or R^{w3} is independently selected from H, F, Cl, Br, I, OH, CN, COOH, C₁₋₄ alkyl, C₁₋₄ alkenyl, C₁₋₄ alkynyl, C₁₋₄ alkoxy, —N(R^{b21})₂, —C(═O)N(R^{b21})₂ or C₃₋₆ cycloalkyl, wherein the alkyl, alkoxy, alkenyl, alkynyl or cycloalkyl is optionally further substituted with 0 to 4 (for example, 0, 1, 2, 3 or 4) substituents selected from H, F, Cl, Br, I, OH, ═O, NH₂, CN, COOH, C₁₋₄ alkyl, C₁₋₄ alkoxy, halogen-substituted C₁₋₄ alkyl, or cyano-substituted C₁₋₄ alkyl;
alternatively, R^{w2} and R^{w3} together with the carbon atoms to which they are attached form C₃₋₆ cycloalkyl or 3- to 8-membered heterocyclyl, wherein the cycloalkyl or heterocyclyl is optionally further substituted with 0 to 4 (for example, 0, 1, 2, 3 or 4) substituents selected from H, F, Cl, Br, I, OH, ═O, NH₂, CN, COOH, C₁₋₄ alkyl, C₁₋₄ alkoxy, halogen-substituted C₁₋₄ alkyl, or cyano-substituted C₁₋₄ alkyl, and the heterocyclyl contains 1 to 4 (for example, 1, 2, 3 or 4) heteroatoms selected from O, S, or N;
each R^{b1} or R^{b2} is independently selected from H, F, Cl, Br, I, ═O, OH, CN, NO₂, COOH, C₁₋₄ alkyl, C₁₋₄ alkenyl, C₁₋₄ alkynyl, C₁₋₄ alkoxy, C₁₋₄ deuterated alkoxy, C₁₋₄ alkylthio, —(CH₂)ₙ—R^{b21}, —OR^{b21}, —N(R^{b21})₂, —C(═O)N(R^{b21})₂, —C(═O)OR^{b21}, —C(═O)R^{b22}, NR^{b21}C(═O)R^{b22}, —NR^{b21}S(═O)₂R^{b22}, C₃₋₆ cycloalkyl, C₆₋₁₀ aryl, 5- to 10-membered heteroaryl or 4- to 10-membered heterocyclyl, wherein the alkyl, alkenyl, alkynyl, alkoxy, alkylthio, cycloalkyl, heterocyclyl, aryl or heteroaryl is optionally further substituted with 0 to 4 (for example, 0, 1, 2, 3 or 4) substituents selected from H, F, Cl, Br, I, OH, ═O, —N(R^{b21})₂, CN, COOH, C₁₋₄ alkyl, C₁₋₄ alkoxy, halogen-substituted C₁₋₄ alkyl, cyano-substituted C₁₋₄ alkyl, C₃₋₆ cycloalkyl, 5- to 10-membered heteroaryl or 4- to 10-membered heterocyclyl, and the heteroaryl or heterocyclyl contains 1 to 4 (for example, 1, 2, 3 or 4) heteroatoms selected from O, S, or N;
each R^{b3} is independently selected from H, F, Cl, Br, I, ═O, OH, CN, COOH, C₁₋₄ alkyl, C₁₋₄ alkenyl, C₁₋₄ alkynyl, C₁₋₄ alkoxy, C₁₋₄ alkylthio, —(CH₂)ₙ—R^{b21}, —OR^{b21}, —N(R^{b21})₂, —C(═O)N(R^{b21})₂, —NR^{b21}C(═O)R^{b22}, —S(═O)₂N(R^{b21})₂, —NR^{b21}S(═O)₂R^{b22}, —C(═O)R^{b22}, or —S(═O)₂R^{b22}, wherein the alkyl, alkoxy, alkenyl, alkynyl or alkylthio is optionally further substituted with 0 to 4 (for example, 0, 1, 2, 3 or 4) substituents selected from H, F, Cl, Br, I, OH, ═O, —N(R^{b21})₂, CN, COOH, C₁₋₄ alkyl, C₁₋₄ alkoxy, halogen-substituted C₁₋₄ alkyl, or cyano-substituted C₁₋₄ alkyl;
each R^{b21} is independently selected from H, C₁₋₄ alkyl, C₃₋₆ cycloalkyl, C₆₋₁₀ aryl, 5- to 10-membered heteroaryl or 4- to 10-membered heterocyclyl, wherein the alkyl, cycloalkyl, aryl, heteroaryl or heterocyclyl is optionally further substituted with 0 to 4 (for example, 0, 1, 2, 3 or 4) substituents selected from H, F, Cl, Br, I, OH, ═O, NH₂, CN, CF₃, COOH, C₁₋₄ alkyl, C₃₋₆ cycloalkyl, or C₁₋₄ alkoxy, and the heteroaryl or heterocyclyl contains 1 to 4 (for example, 1, 2, 3 or 4) heteroatoms selected from O, S, or N;
each R^{b22} is independently selected from H, C₁₋₄ alkyl, C₁₋₄ alkenyl, C₁₋₄ alkynyl, C₁₋₄ alkoxy, or C₃₋₆ cycloalkyl, wherein the alkyl, alkoxy, cycloalkyl, alkenyl, or alkynyl is optionally further substituted with 0 to 4 (for example, 0, 1, 2, 3 or 4) substituents selected from H, F, Cl, Br, I, OH, ═O, NH₂, CN, CF₃, COOH, C₁₋₄ alkyl, C₃₋₆ cycloalkyl, or C₁₋₄ alkoxy;
K is selected from

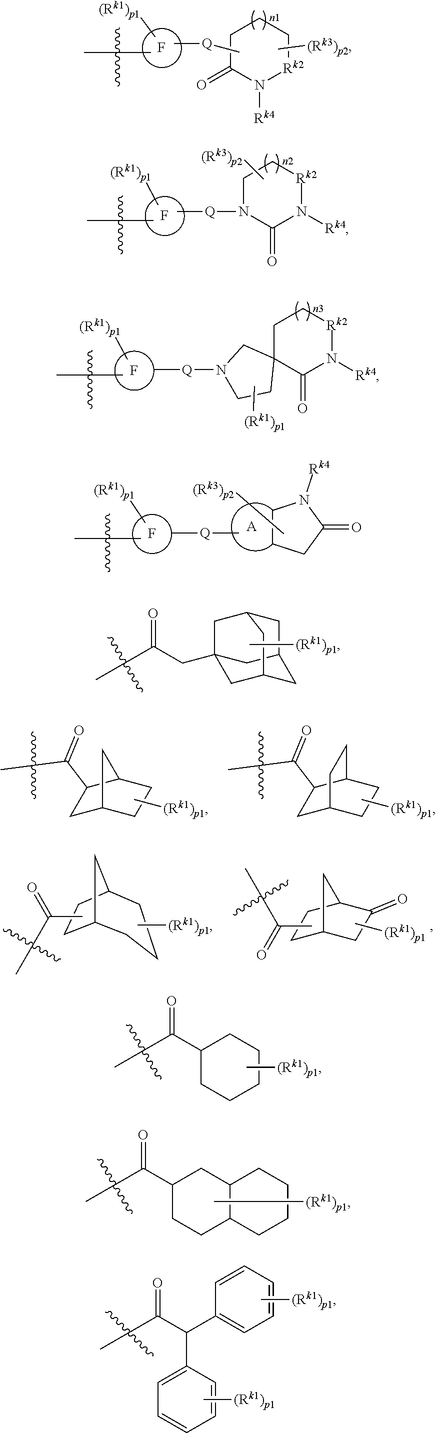

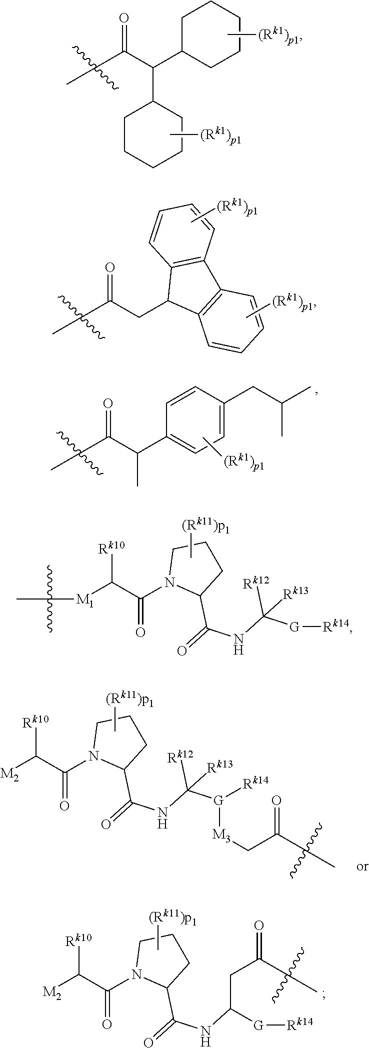

each Q is independently selected from a bond, —O—, —S—, —CH₂—, —NR^{q}—, —CO—, —NR^{q}CO—, —CONR^{q}— or 3- to 12-membered heterocyclyl, wherein the heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
R^{q} is selected from H or C₁₋₆ alkyl;
A is selected from C₃₋₁₀ carbocyclyl, C₆₋₁₀ aryl, 3- to 10-membered heterocyclyl or 5- to 10-membered heteroaryl, wherein the heterocycle or heteroaryl contains 1 to 4 (for example, 1, 2, 3 or 4) heteroatoms selected from O, S, or N;
each F is independently selected from C₃₋₂₀ carbocyclyl, C₆₋₂₀ aryl, 3- to 20-membered heterocyclyl or 5- to 20-membered heteroaryl, wherein the heterocyclyl or heteroaryl contains 1 to 4 (for example, 1, 2, 3 or 4) heteroatoms selected from O, S, or N;
each R^{k2} is independently selected from a bond, —CO—, —SO₂—, —SO— or —C(R^{k3})₂—;
each R^{k1} is independently selected from H, F, Cl, Br, I, OH, ═O, NH₂, CN, COOH, CONH₂, C₁₋₆ alkyl or C₁₋₆ alkoxy, wherein the alkyl or alkoxy is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy;
each R^{k3} is independently selected from H, F, Cl, Br, I, OH, ═O, NH₂, CN, COOH, CONH₂, C₁₋₆ alkyl, C₁₋₆ alkoxy, C₃₋₈ cycloalkyl or 3- to 8-membered heterocyclyl, wherein the alkyl, alkoxy, cycloalkyl or heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
or two R^{k3} together with the carbon atoms or ring backbones to which they are directly attached form 3- to 8-membered carbocycle or 3- to 8-membered heterocycle, wherein the carbocycle or heterocycle is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocycle contains 1 to 4 (for example, 1, 2, 3 or 4) heteroatoms selected from O, S, or N;
each R^{k4} is independently selected from H, OH, NH₂, CN, CONH₂, C₁₋₆ alkyl, C₃₋₈ cycloalkyl or 3- to 8-membered heterocyclyl, wherein the alkyl, cycloalkyl or heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
M₁ is selected from a bond, —CH₂—C(═O)NH— or —C(═O)CH₂NH—;
M₂ is selected from —NHC(═O)—C₁₋₆ alkyl, —NHC(═O)—C₃₋₆ cycloalkyl or 4- to 10-membered heterocyclyl, wherein the alkyl, cycloalkyl or heterocyclyl is optionally further substituted with 0 to 4 (for example, 0, 1, 2, 3 or 4) substituents selected from H, F, Cl, Br, I, ═O, OH, NH₂, C₁₋₄ alkyl or C₁₋₄ alkoxy, and the heterocyclyl contains 1 to 4 (for example, 1, 2, 3 or 4) heteroatoms selected from O, S, or N;
M₃ is selected from —NH— or —O—;
R^{k10} is selected from C₁₋₆ alkyl, wherein the alkyl is optionally further substituted with 0 to 4 (for example, 0, 1, 2, 3 or 4) substituents selected from H, F, Cl, Br, I, ═O, OH, C₁₋₆ alkyl or C₃₋₆ cycloalkyl;
each R^{k11} is independently selected from H, F, Cl, Br, I, ═O, OH, SH, C₁₋₆ alkyl, C₁₋₆ alkoxy or C₁₋₆ alkylthio or —O—C(═O)—C₁₋₆ alkyl, wherein the alkyl, alkoxy or alkylthio is optionally further substituted with 0 to 4 (for example, 0, 1, 2, 3 or 4) substituents selected from H, F, Cl, Br, I, OH, C₁₋₄ alkyl or C₁₋₄ alkoxy;
R^{k12} and R^{k13} are each independently selected from H, C₁₋₆ alkyl or C₃₋₆ cycloalkyl, wherein the alkyl or cycloalkyl is optionally further substituted with 0 to 4 (for example, 0, 1, 2, 3 or 4) substituents selected from H, F, Cl, Br, I, ═O, OH, NH₂, C₁₋₄ alkyl or C₁₋₄ alkoxy;
R^{k14} is selected from 5- to 6-membered heteroaryl, wherein the heteroaryl is optionally further substituted with 0 to 4 (for example, 0, 1, 2, 3 or 4) substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 (for example, 1, 2, 3 or 4) heteroatoms selected from N, O, or S;
G is selected from 6- to 10-membered aryl or 5- to 10-membered heteroaryl, wherein the aryl or heteroaryl is optionally further substituted with 0 to 4 (for example, 0, 1, 2, 3 or 4) substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 (for example, 1, 2, 3 or 4) heteroatoms selected from N, O, or S;
n, r, and t are each independently selected from 0, 1, 2, 3 or 4;
m1, m2, m3, n1, n2, and n3 are each independently selected from 0, 1, 2 or 3;
each p1 or p2 is independently selected from 0, 1, 2, 3, 4 or 5.

As a second embodiment of the present invention, the above-mentioned compound as represented by general formula (I) or the stereoisomer, deuterated compound, solvate, prodrug, metabolite, pharmaceutically acceptable salt or co-crystal thereof, wherein
L is selected from -Cy1-Ak1-Cy2-Ak2-Cy3-Ak3-Cy4-Ak4-;
Ak1, Ak2, Ak3 and Ak4 are each independently selected from —(CH₂)_{q}—, O, —(CH₂)_{q}—NR^{L}, NR^{L}C(═O), C(═O), C(═O)—(CH₂)_{q}—NR^{L}, —C≡C— or a bond;
each q is independently selected from 0, 1, 2, 3 or 4;
each R^{L} is independently selected from H or C₁₋₆ alkyl;
each Cy1, Cy2, Cy3 or Cy4 is independently selected from a bond, 4- to 7-membered mono-heterocyclic ring, 4- to 10-membered fused heterocyclic ring, 5- to 12-membered spiro-heterocyclic ring, 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 (for example, 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 (for example, 1, 2, 3 or 4) heteroatoms selected from O, S, or N;
provided that at least one of Cy1, Cy2, Cy3, and Cy4 cannot be a bond, and when three of them are selected from a bond, the remaining one cannot be selected from triazolyl;
the definitions of other groups are the same as those in the first embodiment of the present invention.

As a third embodiment of the present invention, the above-mentioned compound as represented by general formula (I) or the stereoisomer, deuterated compound, solvate, prodrug, metabolite, pharmaceutically acceptable salt or co-crystal thereof, wherein
Cy1, Cy2, Cy3 and Cy4 are each independently selected from a bond, 4- to 7-membered nitrogen-containing mono-heterocyclic ring, 4- to 10-membered nitrogen-containing fused heterocyclic ring, 5- to 12-membered nitrogen-containing spiro-heterocyclic ring, 7- to 10-membered nitrogen-containing bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the mono-heterocyclic ring, fused heterocyclic ring, bridged-heterocyclic ring, spiro-heterocyclic ring, cycloalkyl, aryl or heteroaryl is optionally further substituted with 0 to 4 (for example, 0, 1, 2, 3 or 4) substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring, spiro-heterocyclic ring or heteroaryl contains 1 to 4 (for example, 1, 2, 3 or 4) heteroatoms selected from O, S, or N;
each R^{L} is independently selected from H or C₁₋₄ alkyl;
each R^{w1} is independently selected from H, methyl, ethyl, isopropyl, cyclopropyl or cyclobutyl;
each R^{w2} or R^{w3} is independently selected from H, F, Cl, Br, I, OH, NH₂, CN, COOH, CONH₂, methyl, ethyl, isopropyl, ethenyl, ethynyl, methoxy, ethoxy, propoxy, cyclopropyl or cyclobutyl, wherein the methyl, ethyl, isopropyl, ethenyl, ethynyl, methoxy, ethoxy, propoxy, cyclopropyl or cyclobutyl is optionally further substituted with 0 to 4 (for example, 0, 1, 2, 3 or 4) substituents selected from H, F, Cl, Br, I, OH, or NH₂;
alternatively, R^{w2} and R^{w3} together with the carbon atoms to which they are attached form cyclopropyl, cyclobutyl, cyclopentyl, azetidinyl, oxetanyl, pyrrolidinyl, or piperidyl, wherein the cyclopropyl, cyclobutyl, cyclopentyl, azetidinyl, oxetanyl, pyrrolidinyl, or piperidyl is optionally further substituted with 0 to 4 (for example, 0, 1, 2, 3 or 4) substituents selected from H, F, Cl, Br, I, OH, NH₂, CN, CF₃, or CHF₂;
each R^{b1} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, deuterated methoxy, methylthio, ethylthio, morpholinyl, piperazinyl, pyrrolidinyl, piperidyl or oxazolidinyl, wherein the methoxy, ethoxy, propoxy, isopropyloxy, methylthio, or ethylthio is optionally further substituted with 0 to 4 (for example, 0, 1, 2, 3 or 4) substituents selected from H, F, Cl, Br, I, OH, or NH₂;
each R¹² is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, morpholinyl, piperazinyl, pyrrolidinyl, piperidyl or oxazolidinyl, wherein the methyl, ethyl, ethenyl, ethynyl, methoxy, ethoxy, propoxy, isopropyloxy, morpholinyl, piperazinyl, pyrrolidinyl, piperidyl or oxazolidinyl is optionally further substituted with 0 to 4 (for example, 0, 1, 2, 3 or 4) substituents selected from H, F, Cl, Br, I, OH, or NH₂;
each R^{b3} is independently selected from H, F, Cl, Br, I, ═O, CF₃, CHF₂, OH, CN, COOH, NH₂, CONH₂, S(═O)₂NH₂, S(═O)₂CH₃, methyl, ethyl, isopropyl, ethenyl, ethynyl, methoxy, ethoxy, propoxy, methylthio or ethylthio, wherein the methyl, ethyl, isopropyl, ethenyl, ethynyl, methoxy, ethoxy, propoxy, methylthio or ethylthio is optionally further substituted with 0 to 4 (for example, 0, 1, 2, 3 or 4) substituents selected from H, F, Cl, Br, I, OH, or NH₂;
r is selected from 1 or 2;
each t is independently selected from 0, 1 or 2;
K is selected from

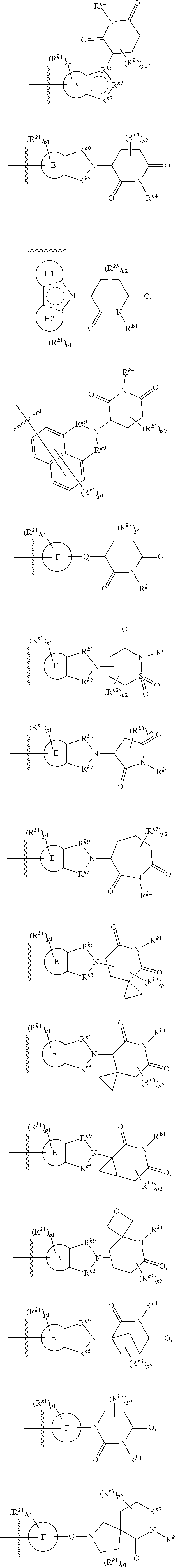

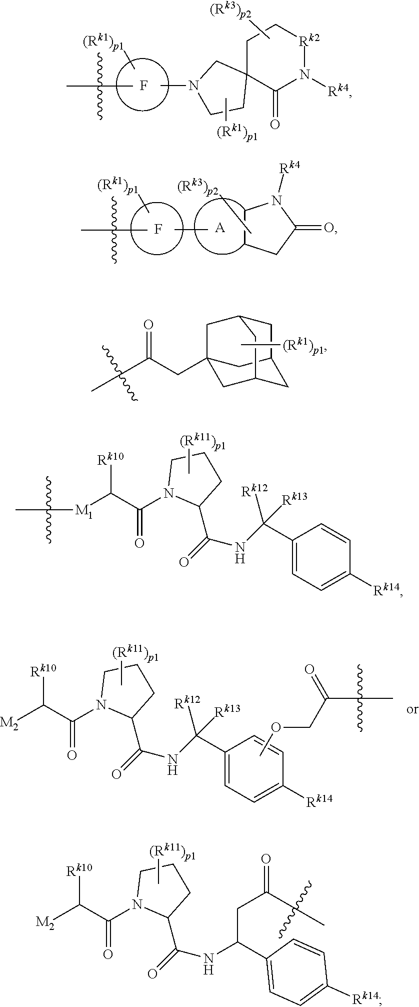

or K is selected from

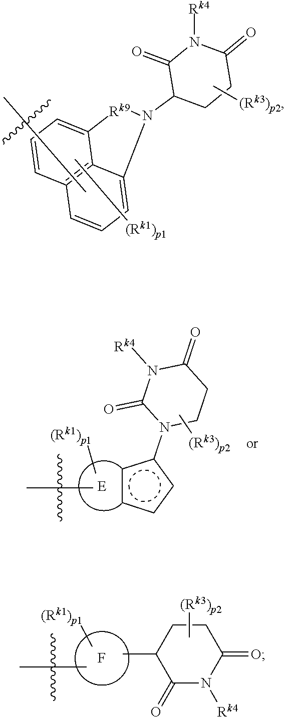

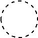

represents a ring selected from an aromatic ring or a non-aromatic ring;
each Q is independently selected from —O—, —S—, —CH₂—, —NR^{q}—, —CO—, —NR^{q}CO—, —CONR^{q}— or 4- to 7-membered heterocyclyl, wherein the heterocyclyl is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocyclyl contains 1 to 4 (for example, 1, 2, 3 or 4) heteroatoms selected from O, S, or N;
R^{q} is selected from H or C₁₋₄ alkyl;
R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 substituents selected from H, F, Cl, Br, I, OH, or NH₂;
or two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 (for example, 0, 1, 2, 3 or 4) substituents selected from H, F, Cl, Br, I, OH, ═O, NH₂, CN, COOH, CONH₂, C₁₋₄ alkyl or C₁₋₄ alkoxy, and the heterocycle contains 1 to 4 (for example, 1, 2, 3 or 4) heteroatoms selected from O, S, or N;
each R^{k4} is independently selected from H, OH, NH₂, CF₃, CN, or C₁₋₄ alkyl;
each R^{k8} is independently selected from CO, CH₂, SO₂₂ or

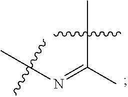 [SMILES: */N=C(\*)C]

each R^{k6} is independently selected from CO, CH, SO, SO₂, CH₂ or N;
each R^{k7} is independently selected from CO, CH, N, CH₂, O, S, N(CH₃) or NH;
each R^{k8} is independently selected from C, N or CH;
each R^{k9} is independently selected from CO, SO₂ or CH₂;
each A, H1 or H2 is independently selected from C₃₋₈ carbocycle, a benzene ring, 4- to 7-membered heterocycle or 5- to 6-membered heteroaryl, wherein the heterocycle or heteroaryl contains 1 to 4 heteroatoms selected from O, S, or N;
each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 (for example, 1, 2, 3 or 4) heteroatoms selected from O, S, or N;
each F is independently selected from 3- to 7-membered monocycloalkyl, 4- to 10-membered fused cycloalkyl, 5- to 12-membered spiro cycloalkyl, 5- to 10-membered bridged cycloalkyl, 4- to 7-membered mono-heterocyclic ring, 4- to 10-membered fused heterocyclic ring, 5- to 12-membered spiro-heterocyclic ring, 5- to 10-membered bridged-heterocyclic ring, C₆₋₁₄ aryl or 5- to 10-membered heteroaryl, wherein the mono-heterocyclic ring, fused heterocyclic ring, spiro-heterocyclic ring, bridged-heterocyclic ring or heteroaryl contains 1 to 4 (for example, 1, 2, 3 or 4) heteroatoms selected from O, S, or N;
the definitions of other groups are the same as those in any one of the first and second embodiments of the present invention.

As a fourth embodiment of the present invention, the above-mentioned compound as represented by general formula (I) or the stereoisomer, deuterated compound, solvate, prodrug, metabolite, pharmaceutically acceptable salt or co-crystal thereof, wherein
R^{L} is selected from H, methyl or ethyl;
each q is independently selected from 0, 1 or 2;
Cy1, Cy2, Cy3 and Cy4 are each independently selected from a bond or one of the following substituted or unsubstituted groups: cyclopropyl, cyclobutyl, cyclopentyl, cyclohexyl, azetidinyl, azacyclopentyl, azacyclohexenyl, piperidine, morpholine, piperazine, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl-spiro-piperidine, azetidinyl-spiro-piperidine, azacyclopentyl-spiro-piperidine, azacyclohexyl-spiro-piperidine,

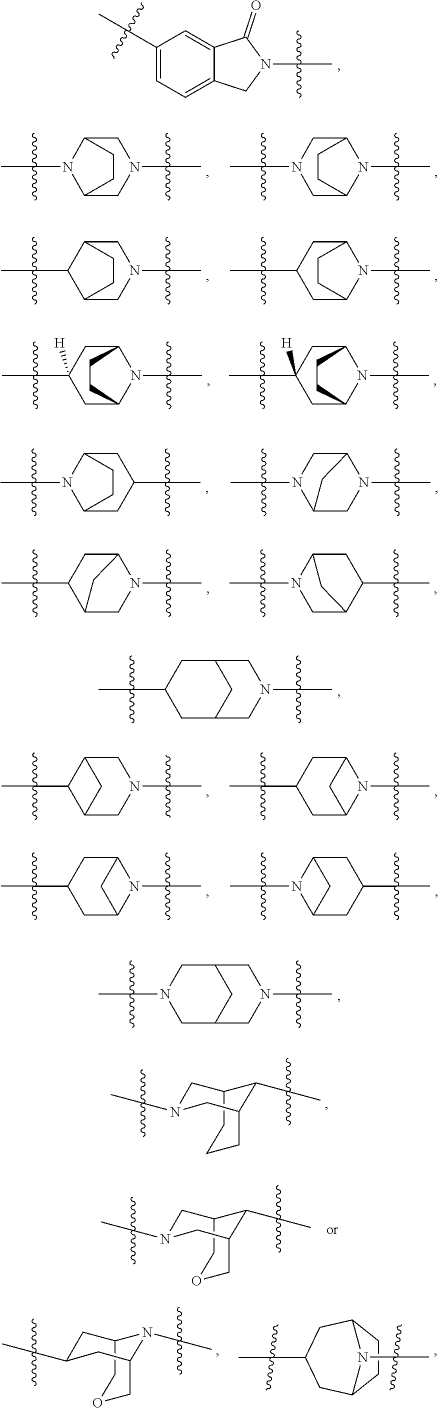

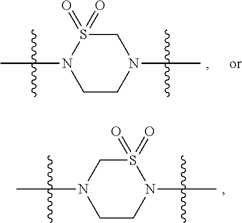

which, when substituted, is optionally further substituted with 0 to 4 (for example, 0, 1, 2, 3 or 4) substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl or C₁₋₄ alkoxy;
B is selected from

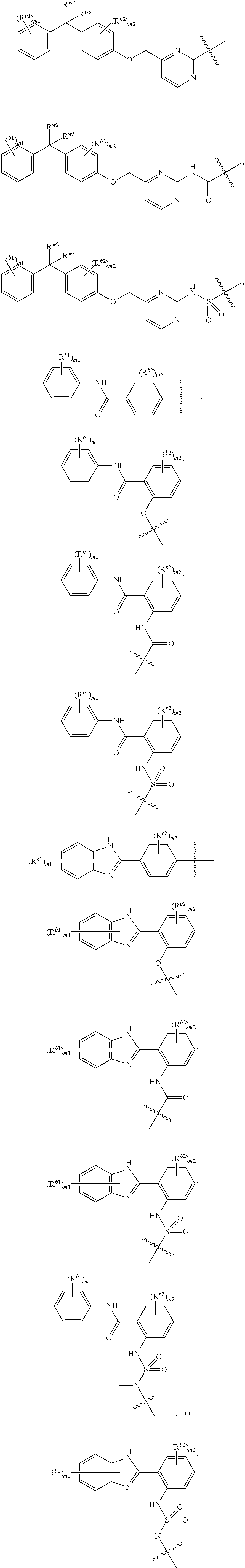

or B is selected from

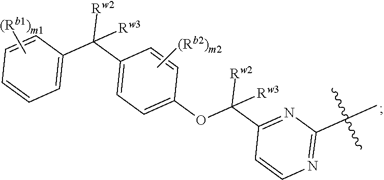

K is selected from

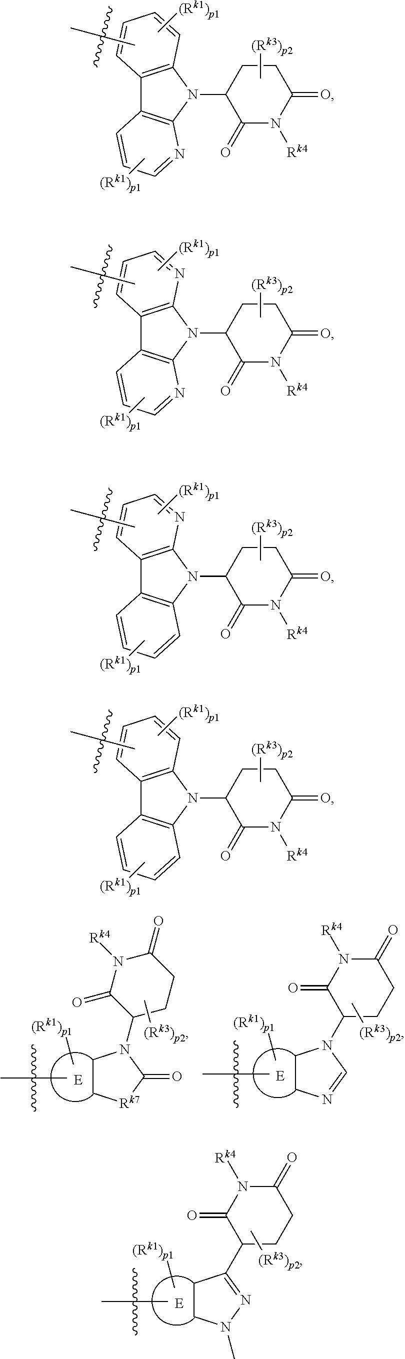

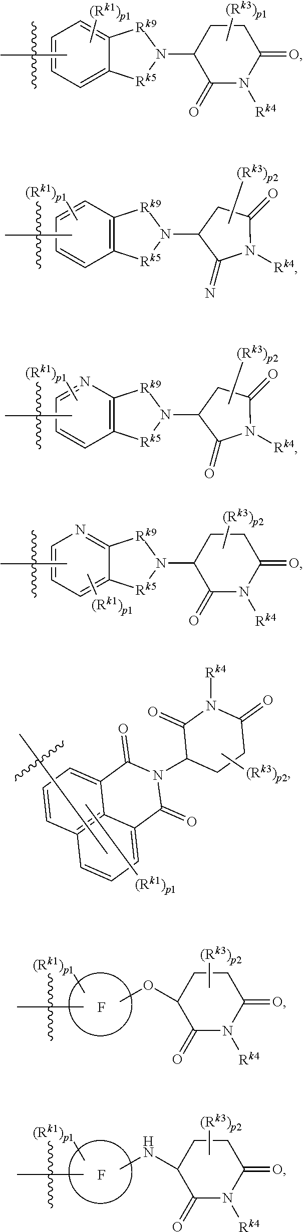

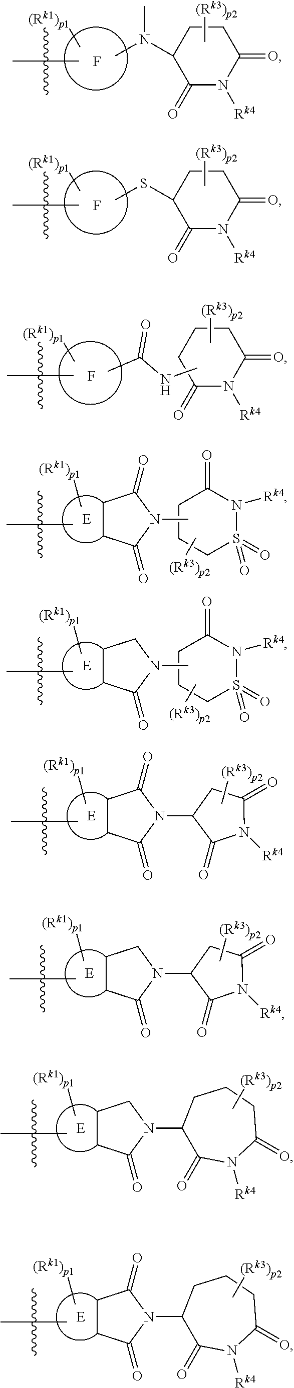

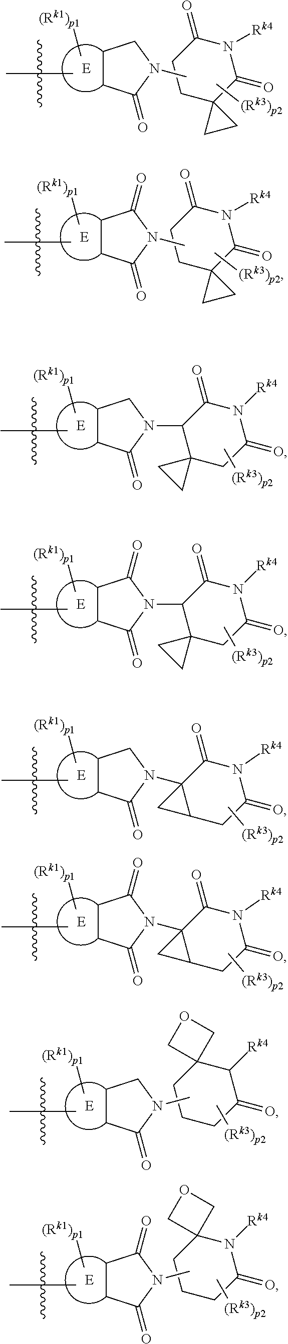

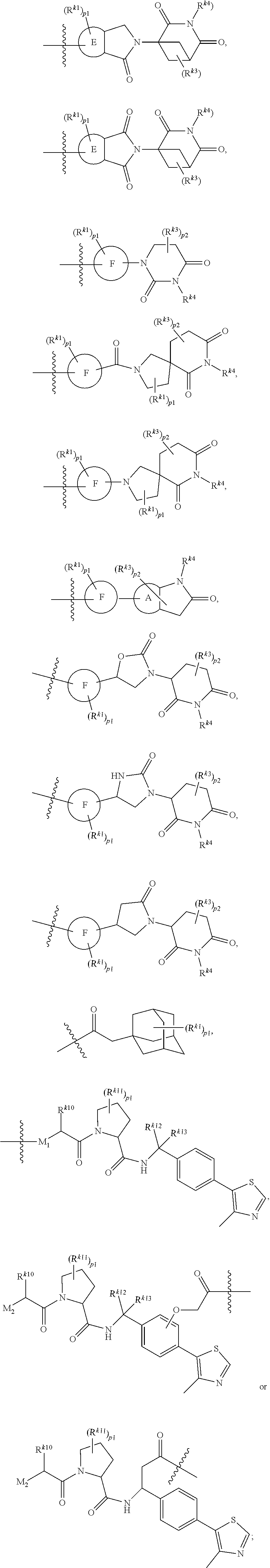

or K is selected from

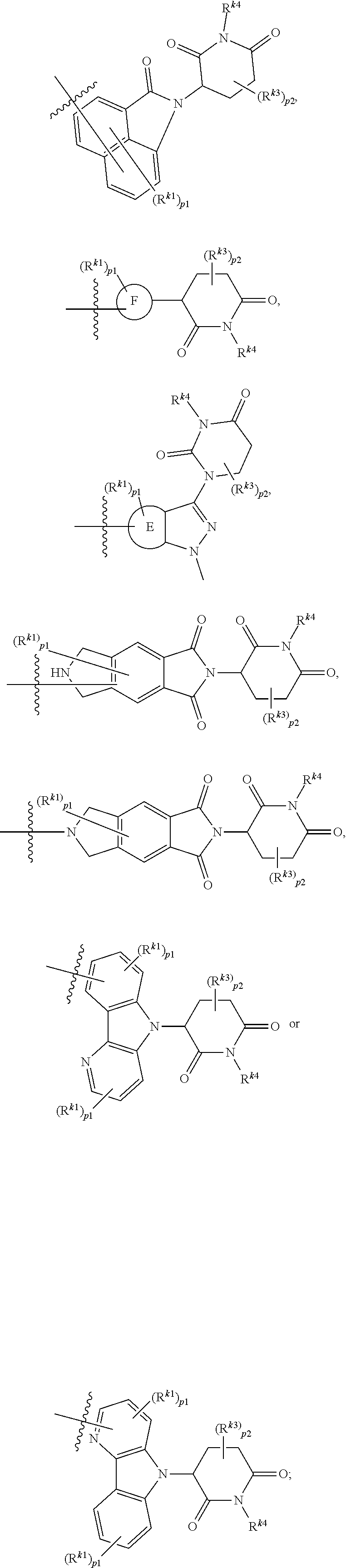

each E is independently selected from phenyl, pyridyl, pyridazinyl, pyrazinyl, pyrimidinyl, pyrrolyl, pyrazolyl, imidazoly, thiazolyl, furyl, thienyl or oxazolyl;
each A is independently selected from phenyl, pyridyl, pyridazinyl, pyrazinyl, pyrimidinyl, pyrrolyl, pyrazolyl, imidazoly, thiazolyl, furyl, thienyl or oxazolyl;
each F is independently selected from cyclopropyl, cyclobutyl, cyclopentyl, cyclohexyl, phenyl, naphthyl, anthryl, phenanthrenyl, azetidinyl, azacyclopentyl, piperidyl, morpholinyl, pyridyl, pyrimidinyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidinyl, benzopyridazinyl, pyrrolopyrrolyl, pyrrolopyridinyl, pyrrolopyrimidinyl, pyrrolopyridazinyl, pyrrolopyrazinyl, imidazopyrimidinyl, imidazopyridinyl, imidazopyrazinyl, imidazopyridazinyl, pyrazolopyridinyl, pyrazolopyrimidinyl, pyrazolopyridazinyl, pyrazolopyrazinyl, pyrimidopyridyl, pyrimidopyrazinyl, pyrimidopyridazinyl, pyrimidopyrimidinyl, pyridopyridyl, pyridopyrazinyl, pyridopyridazinyl, pyridazopyridazinyl, pyridazopyrazinyl or pyrazinopyrazinyl;
each R^{k7} is independently selected from CH₂, O, N(CH₃) or NH;
each p1 or p2 is independently selected from 0, 1 or 2;
the definitions of other groups are the same as those in any one of the first, second and third embodiments of the present invention.

As a fifth embodiment of the present invention, the above-mentioned compound as represented by general formula (I) or the stereoisomer, deuterated compound, solvate, prodrug, metabolite, pharmaceutically acceptable salt or co-crystal thereof, wherein
Cy1, Cy2, Cy3 and Cy4 are each independently selected from a bond or one of the following substituted or unsubstituted groups:

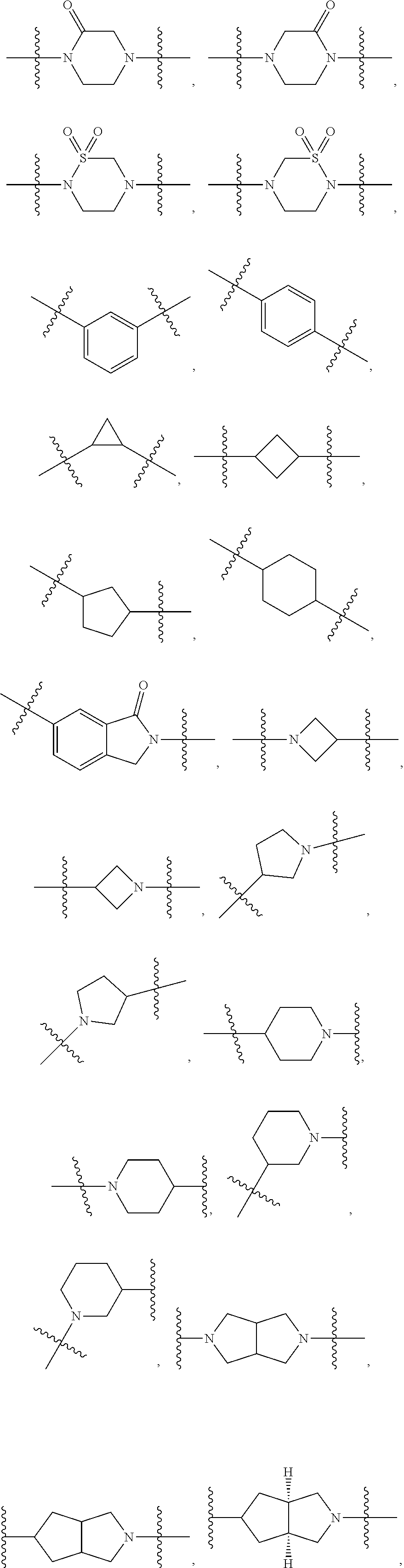

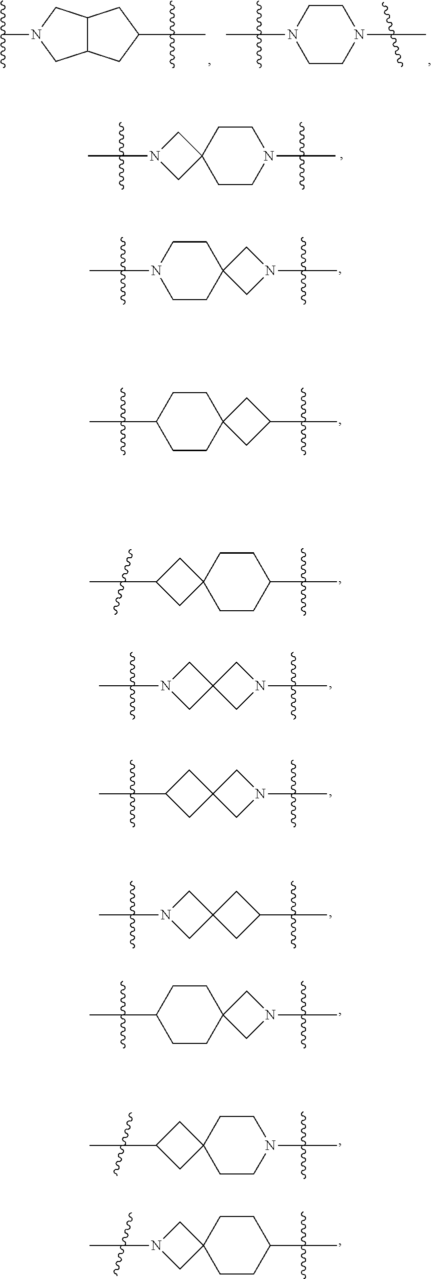

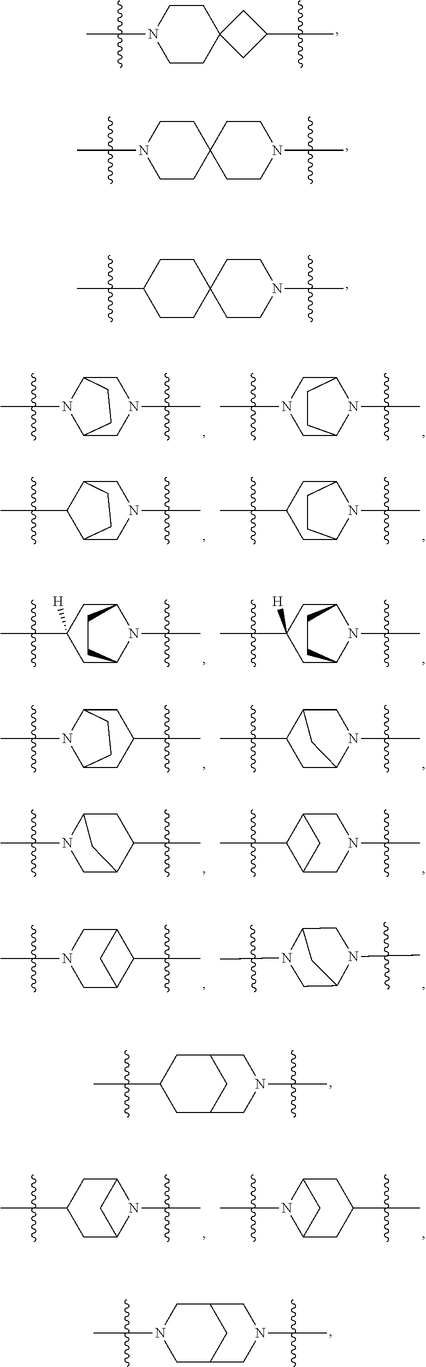

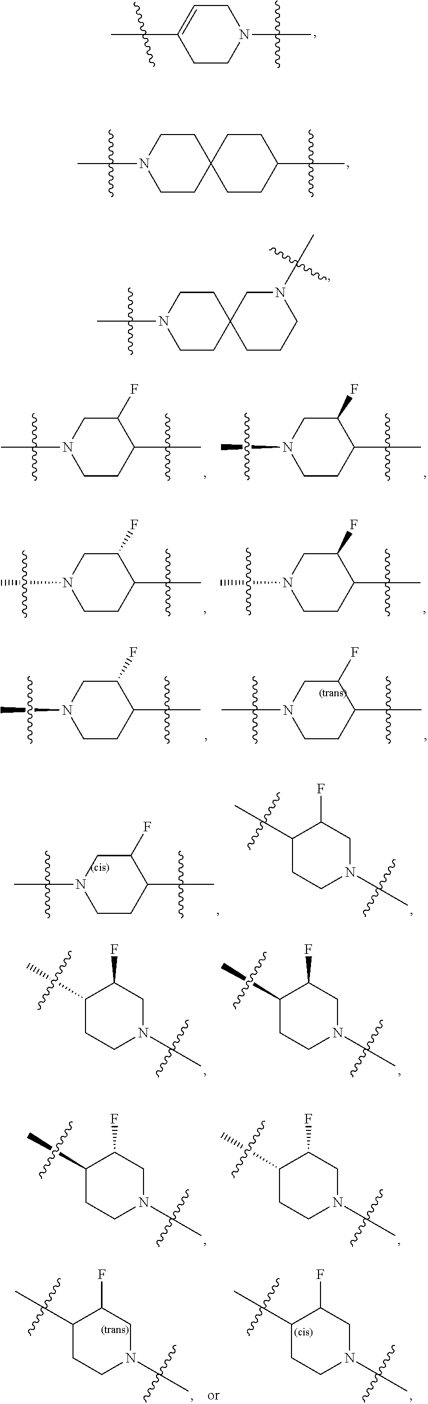

which, when substituted, is optionally further substituted with 0 to 4 (for example, 0, 1, 2, 3 or 4) substituents selected from H, F, CF₃, methyl, ═O, hydroxymethyl, COOH, CN or NH₂;
K is selected from

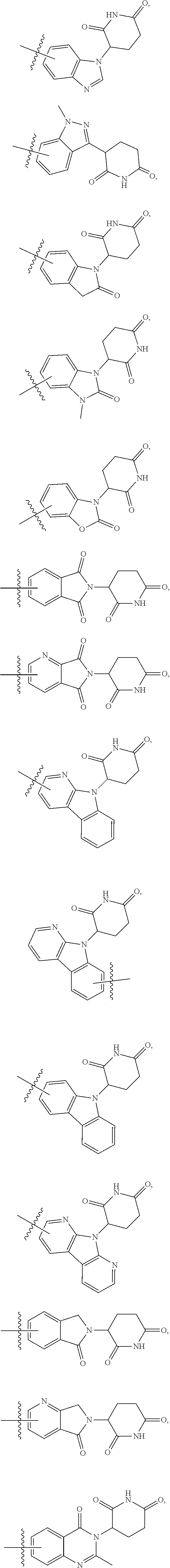

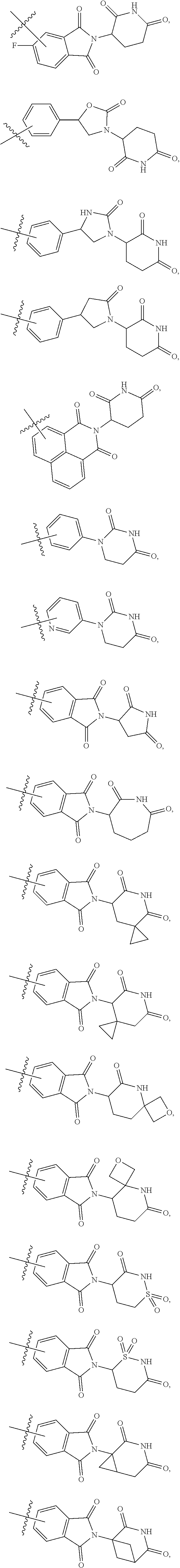

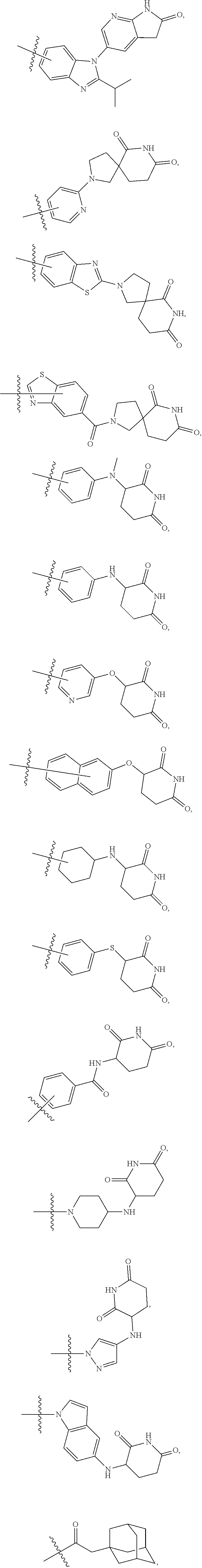

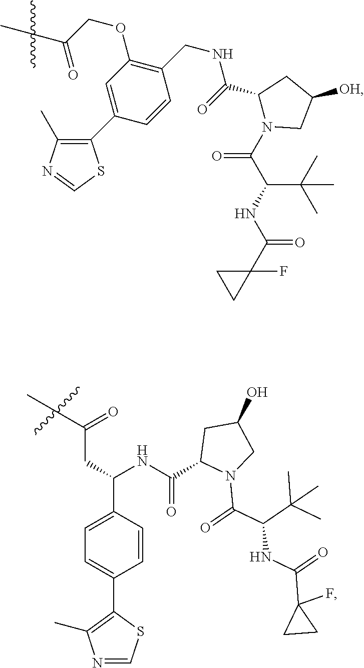 [SMILES: *C(=O)COc1cc(-c2scnc2C)ccc1CNC(=O)[C@@H]1C[C@@H](O)CN1C(=O)[C@@H](NC(=O)C1(F)CC1)C(C)(C)C ; *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1]

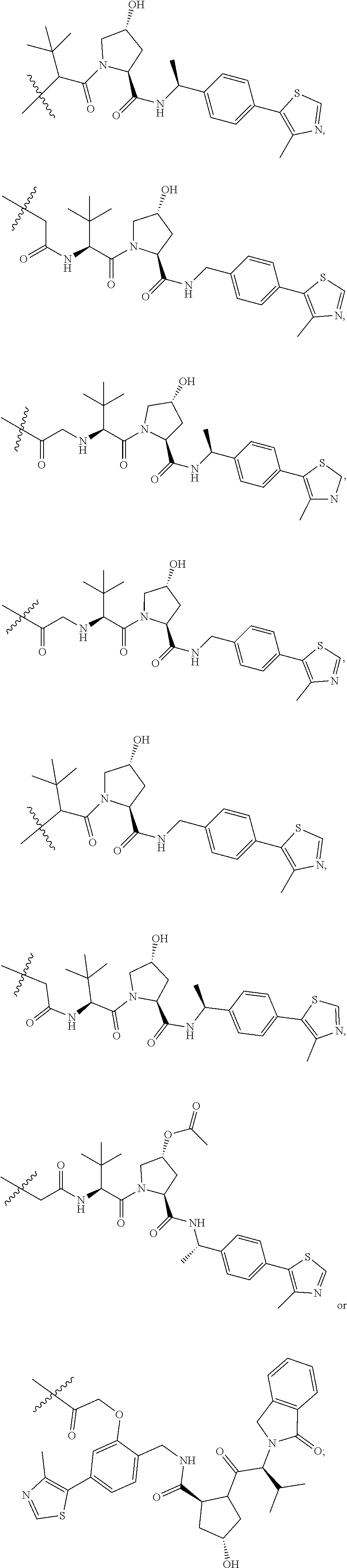

or K is selected from

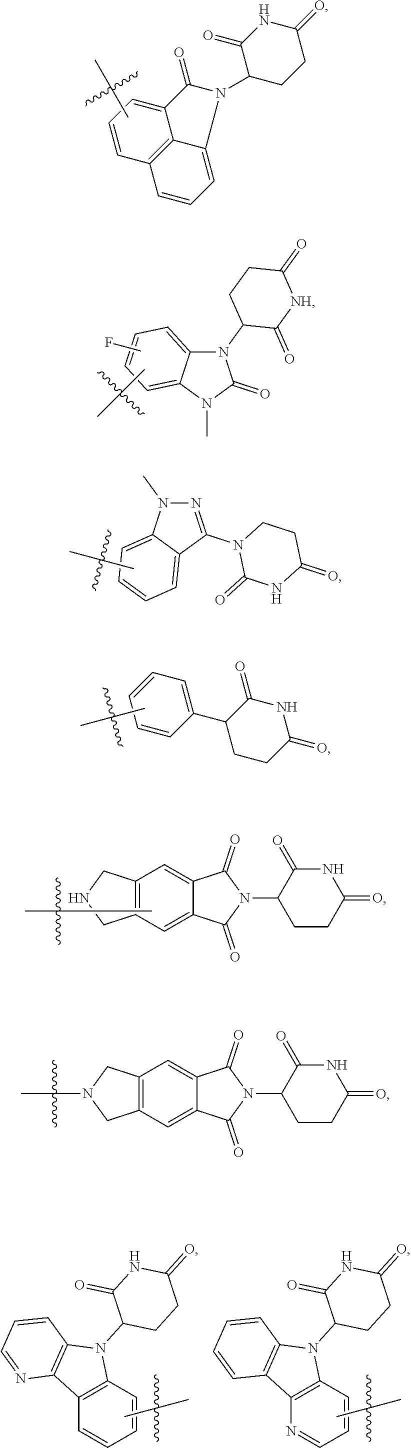

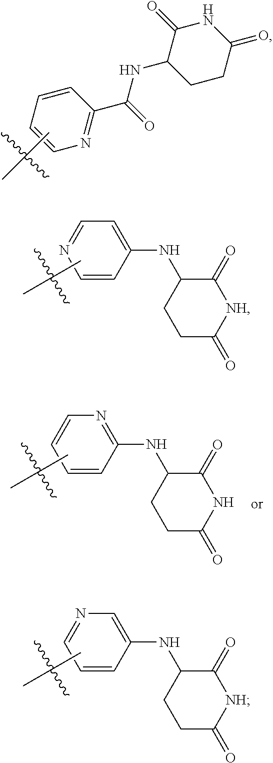

the definitions of other groups are the same as those in any one of the first, second and third embodiments of the present invention.

As a sixth embodiment of the present invention, the above-mentioned compound as represented by general formula (I) or the stereoisomer, deuterated compound, solvate, prodrug, metabolite, pharmaceutically acceptable salt or co-crystal thereof, wherein
L is selected from a bond,

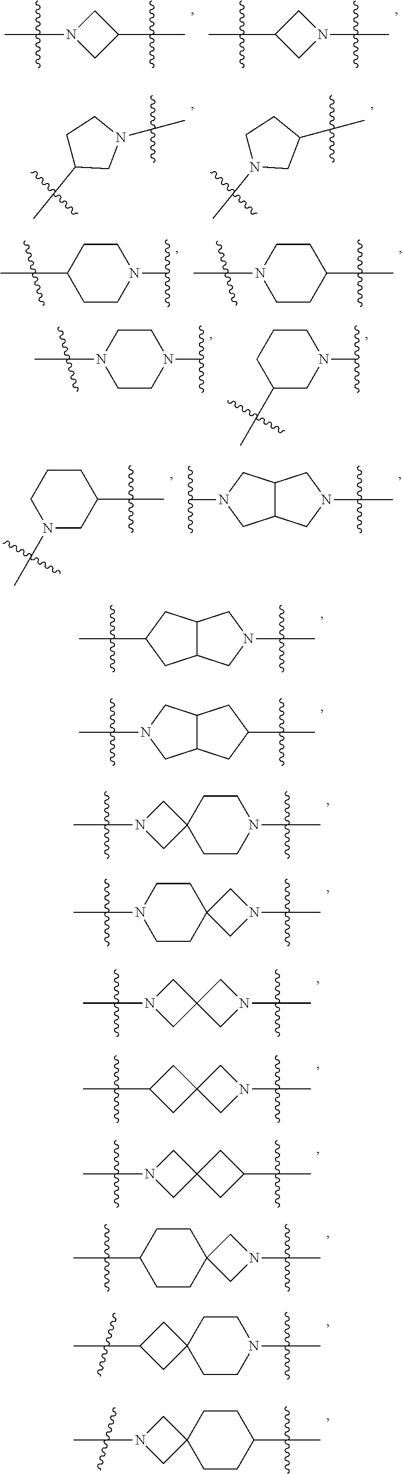

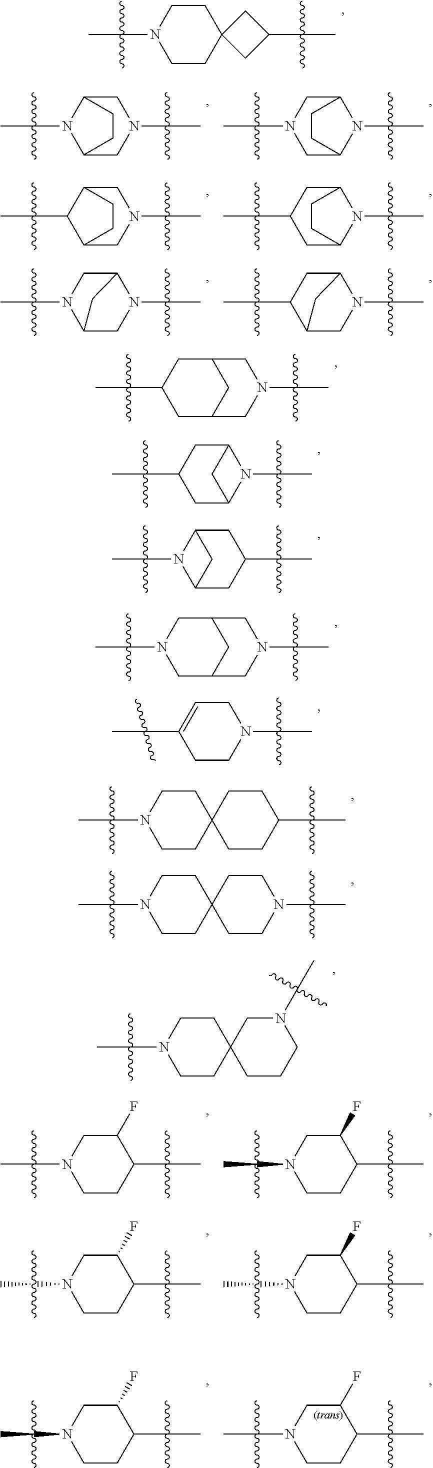

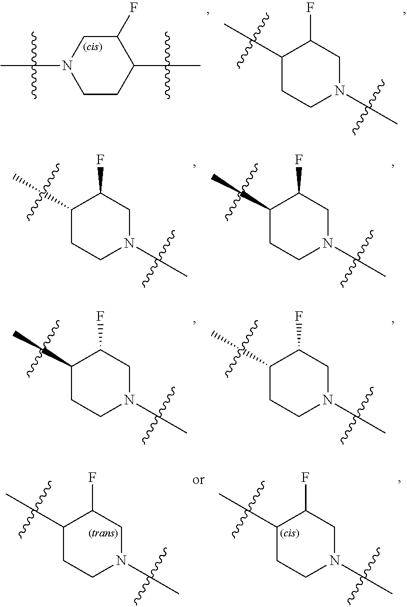

wherein the left side thereof is linked to B;
or L is selected from

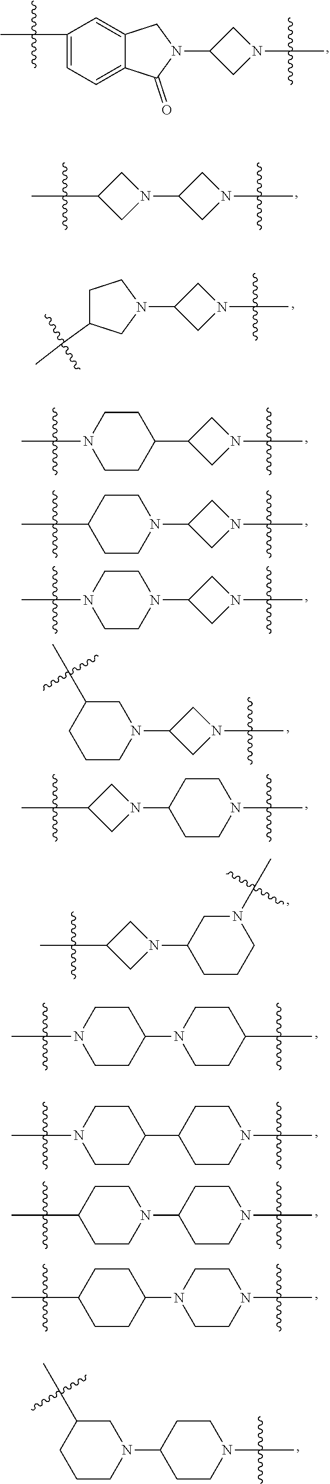

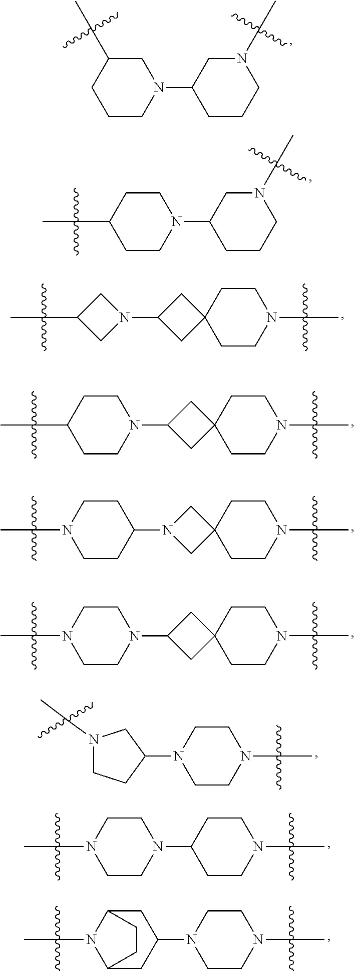

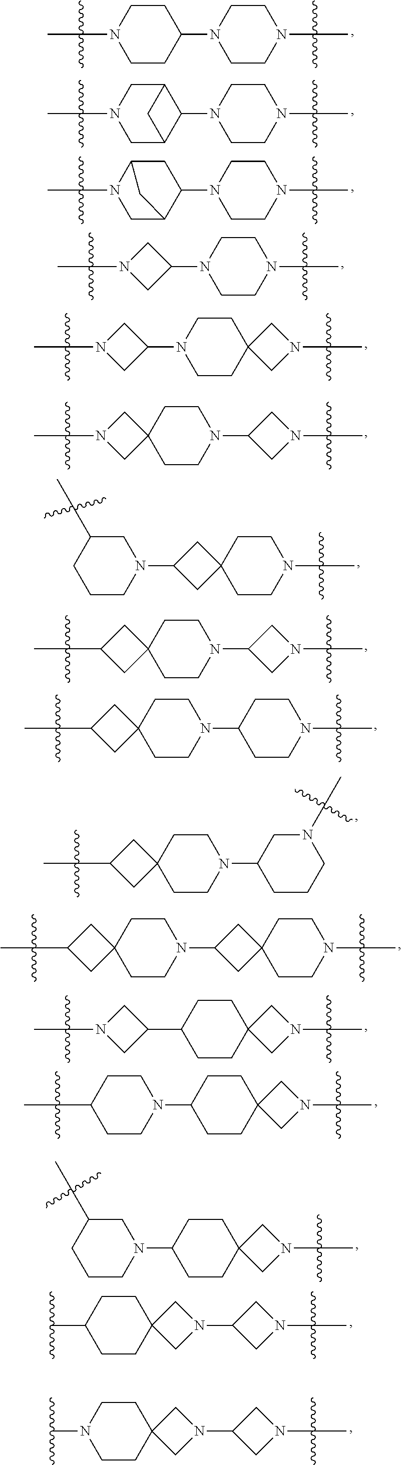

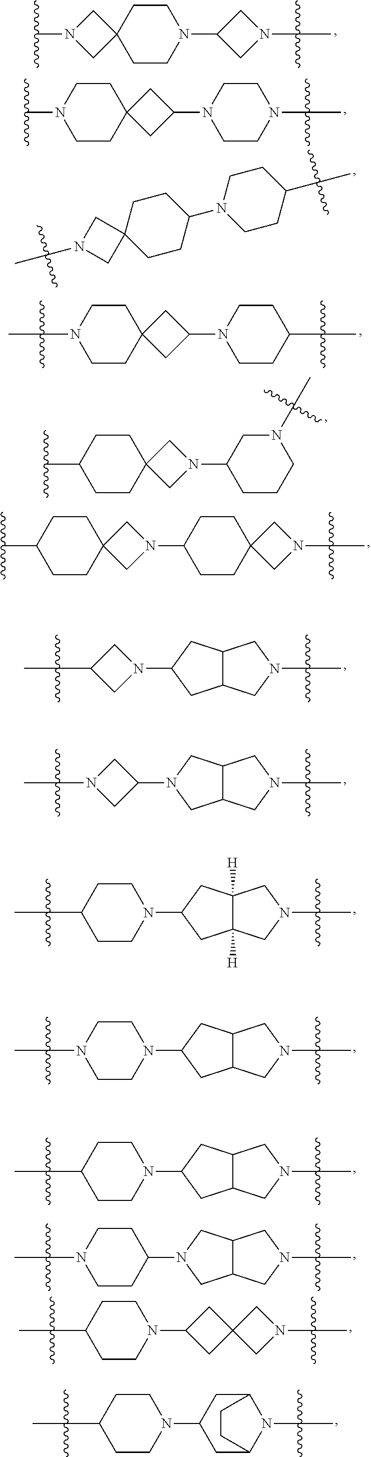

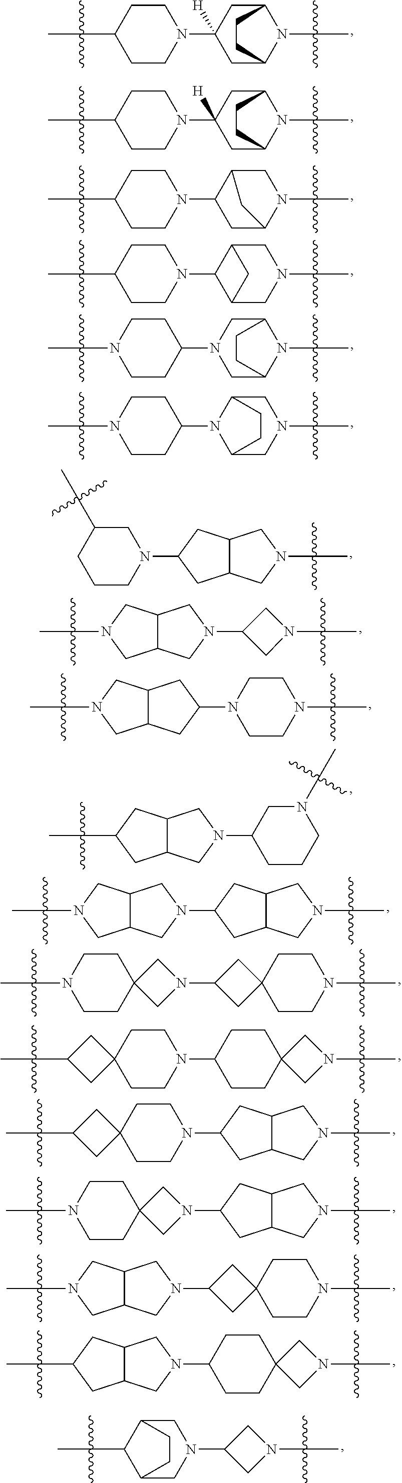

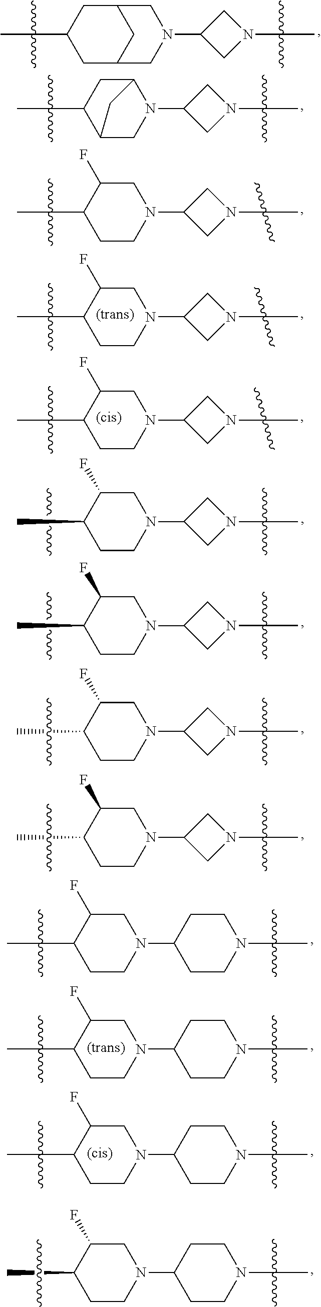

[chemical structure]

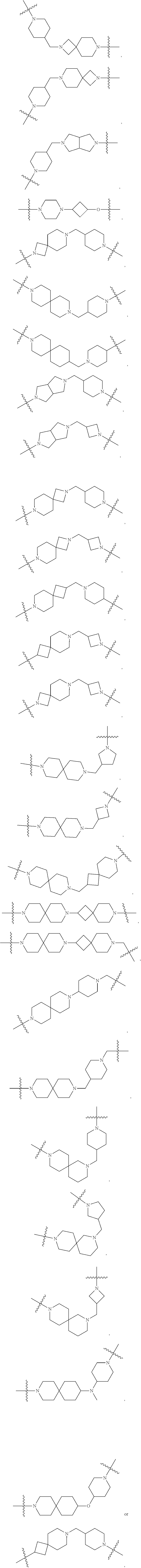

wherein the left side thereof is linked to B;
or L is selected from

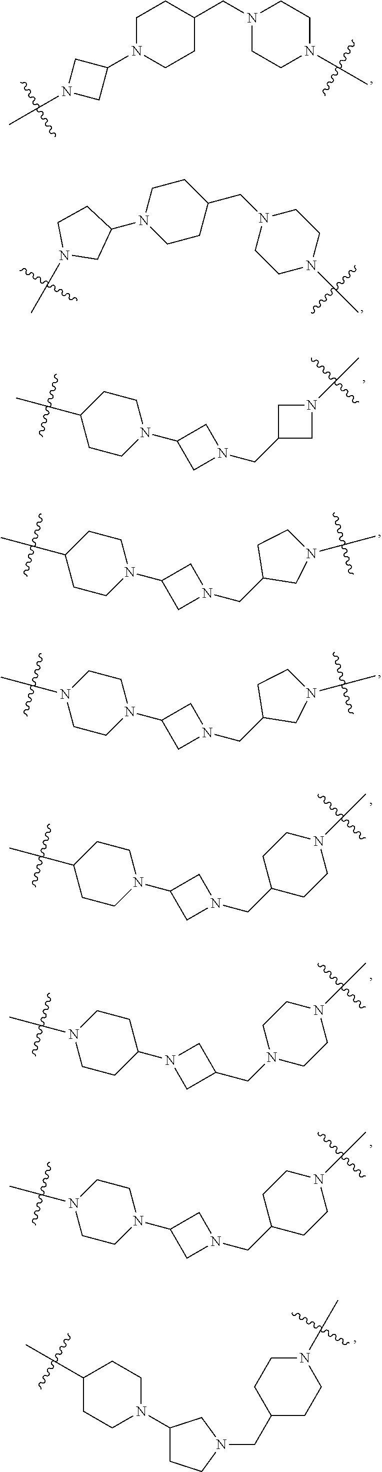

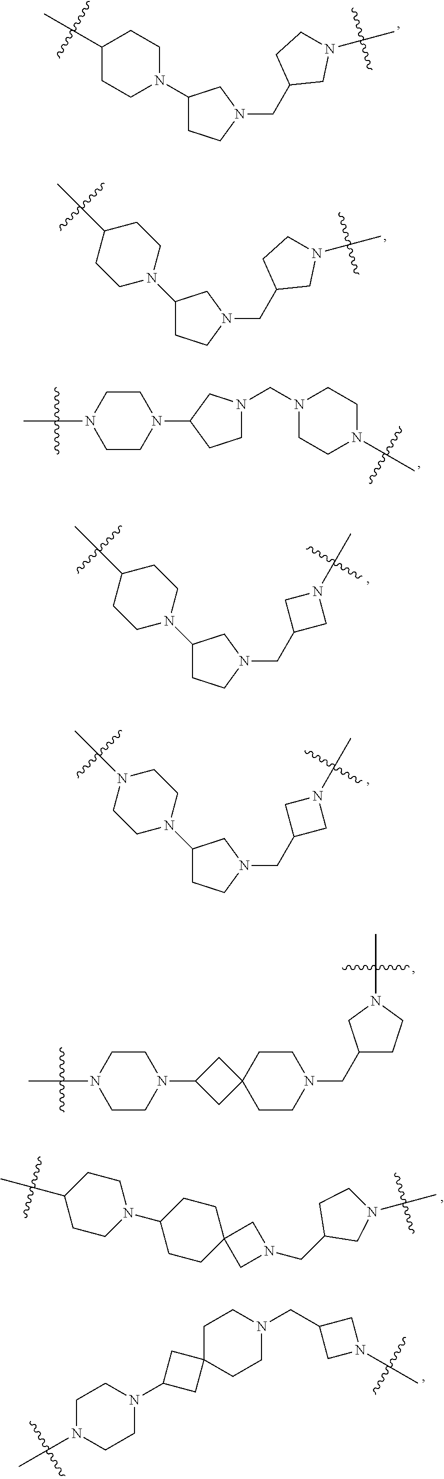

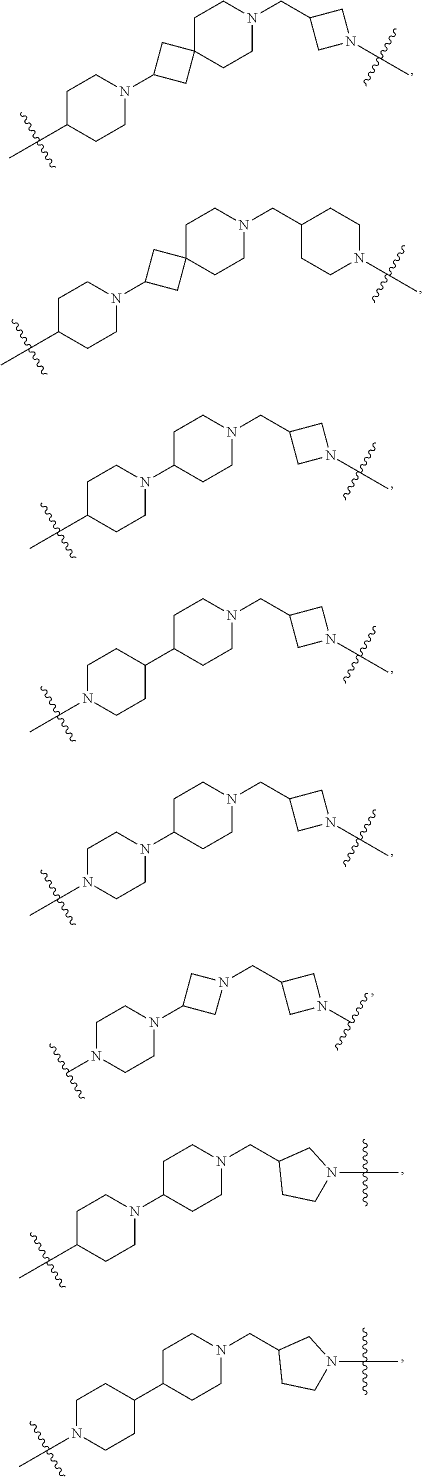

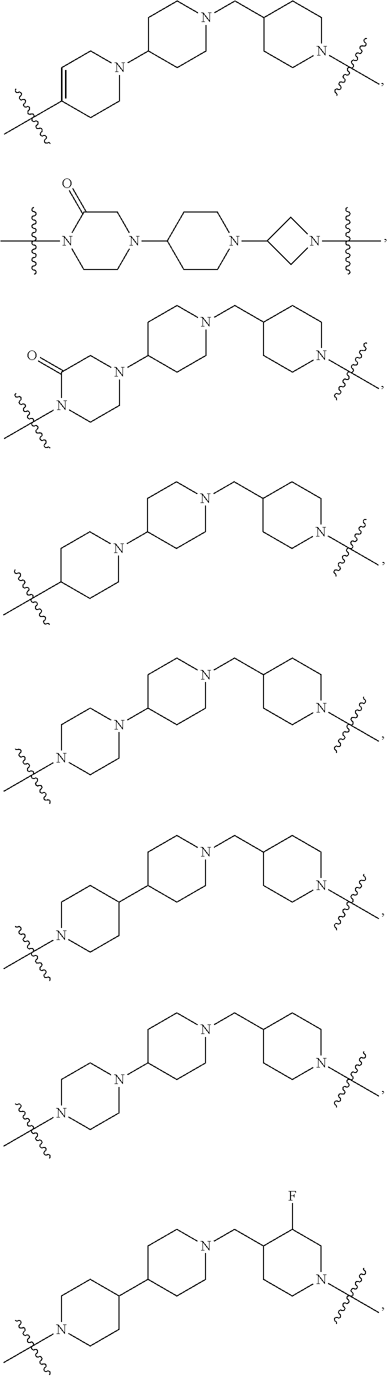

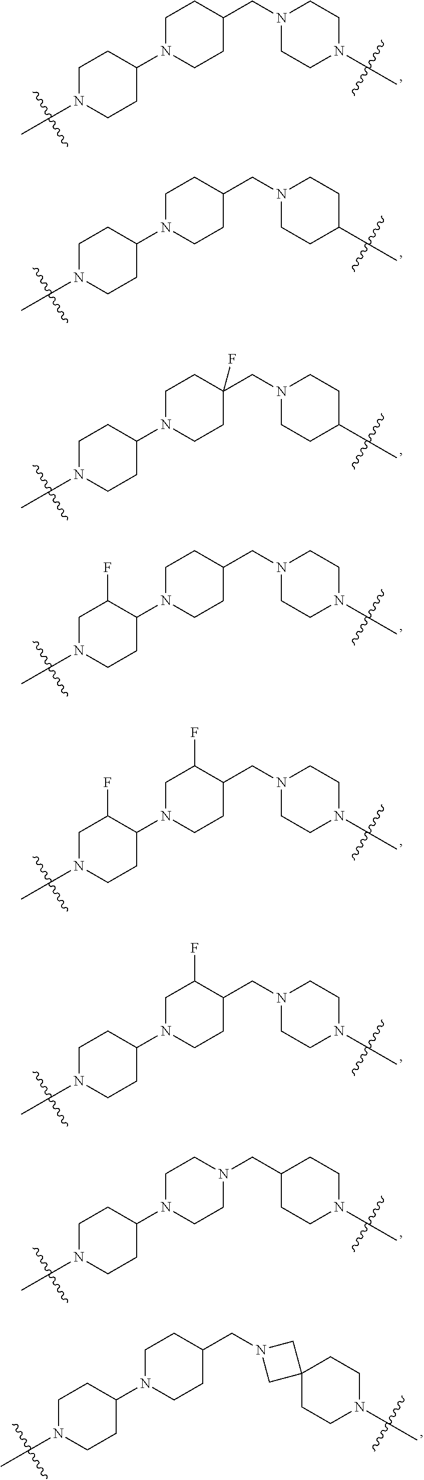

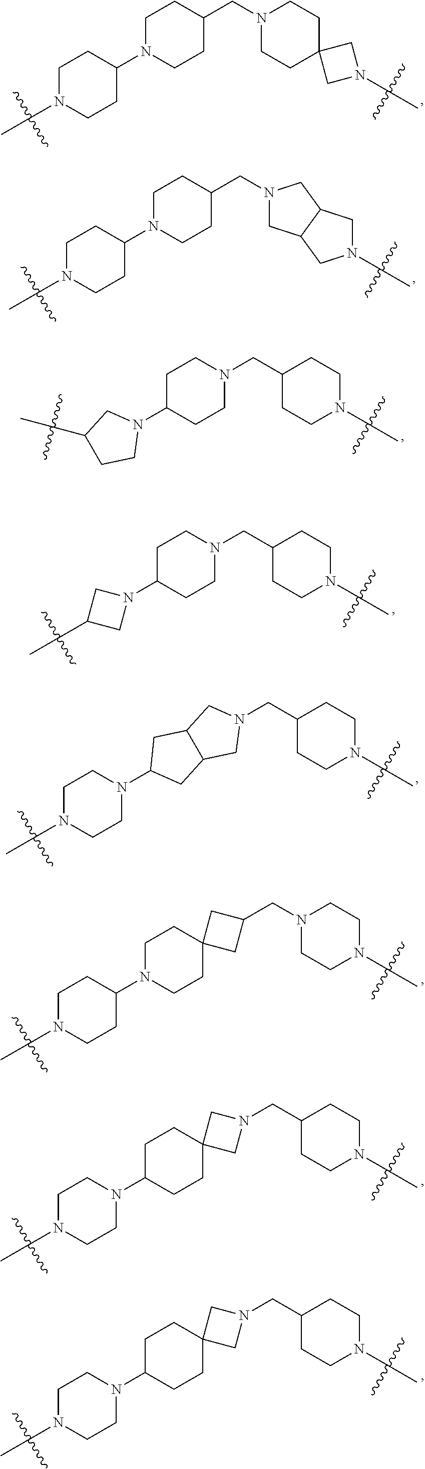

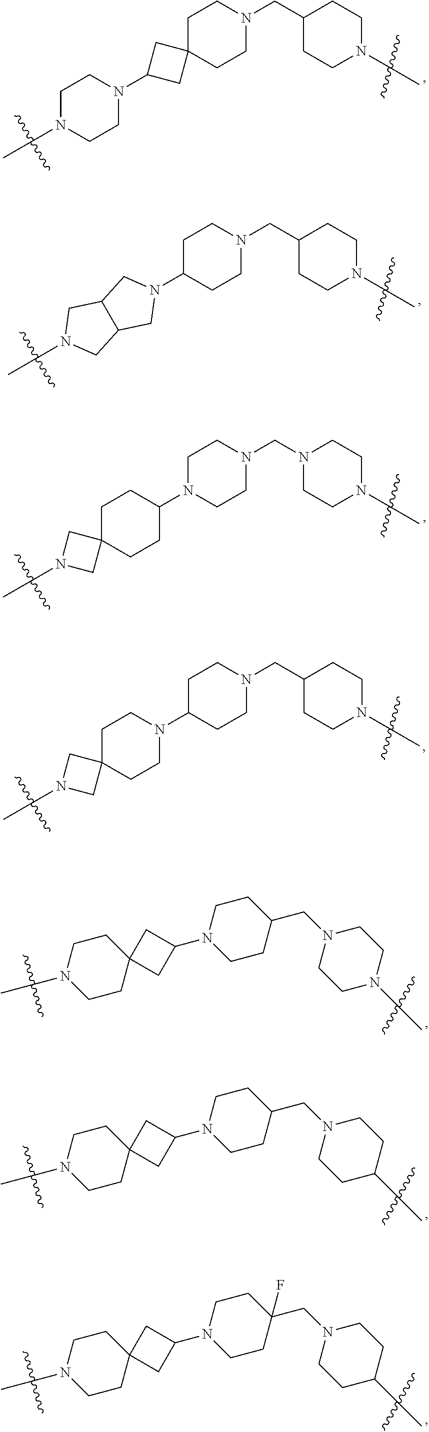

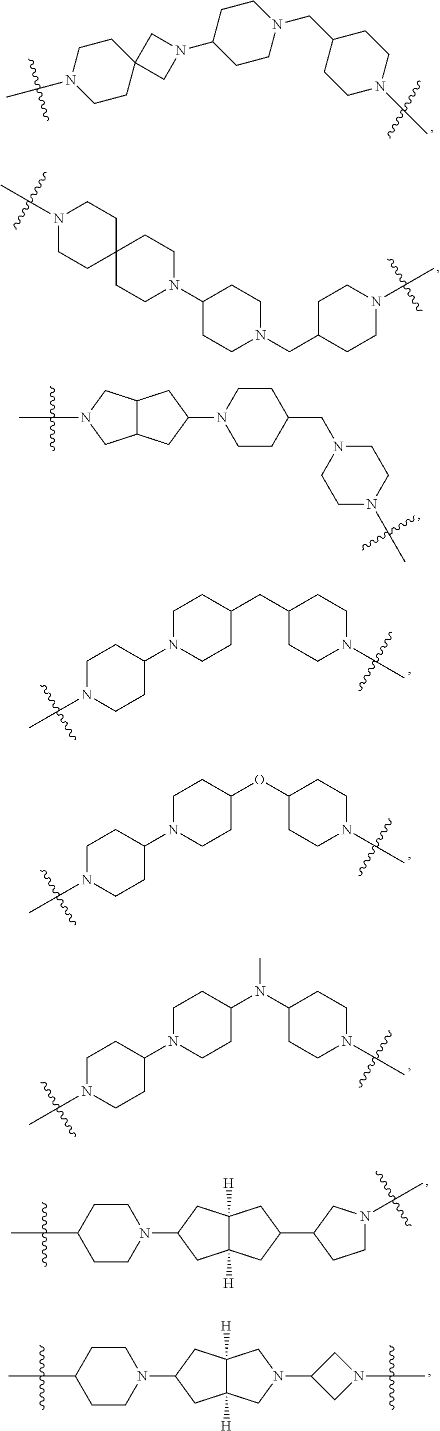

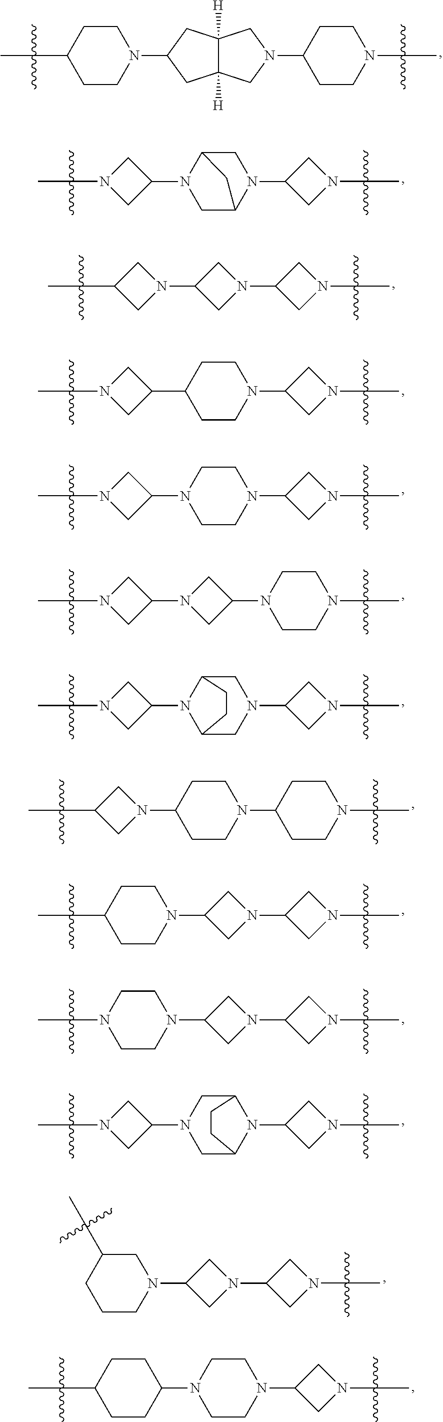

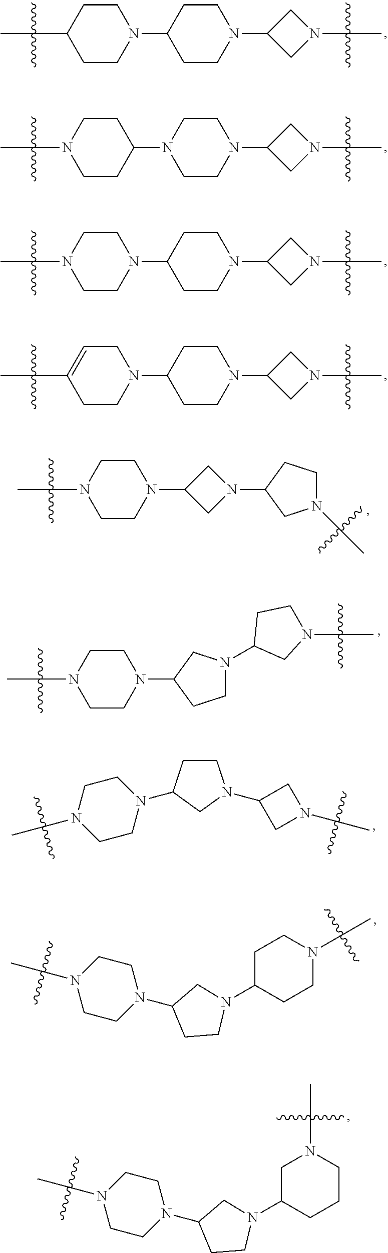

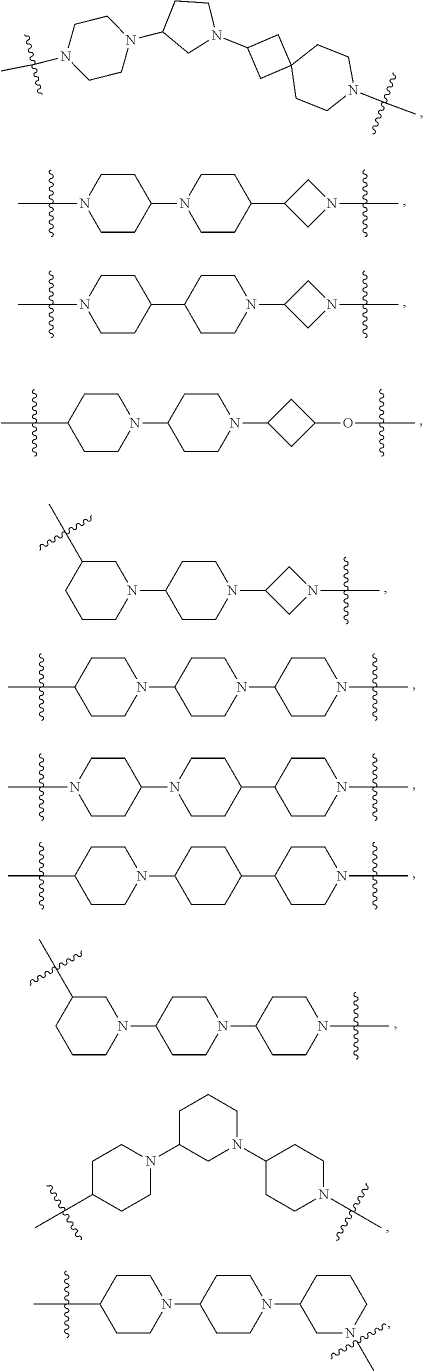

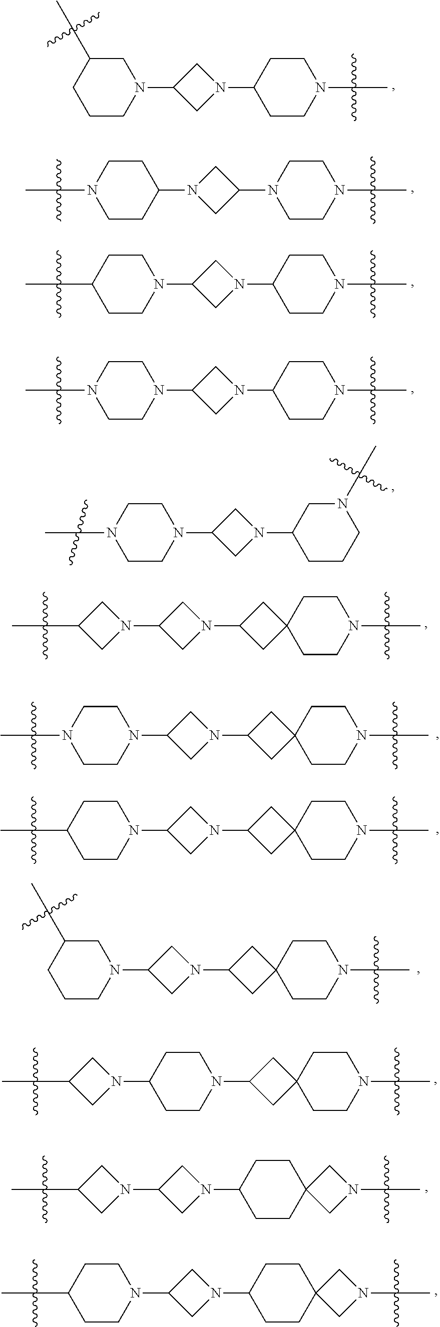

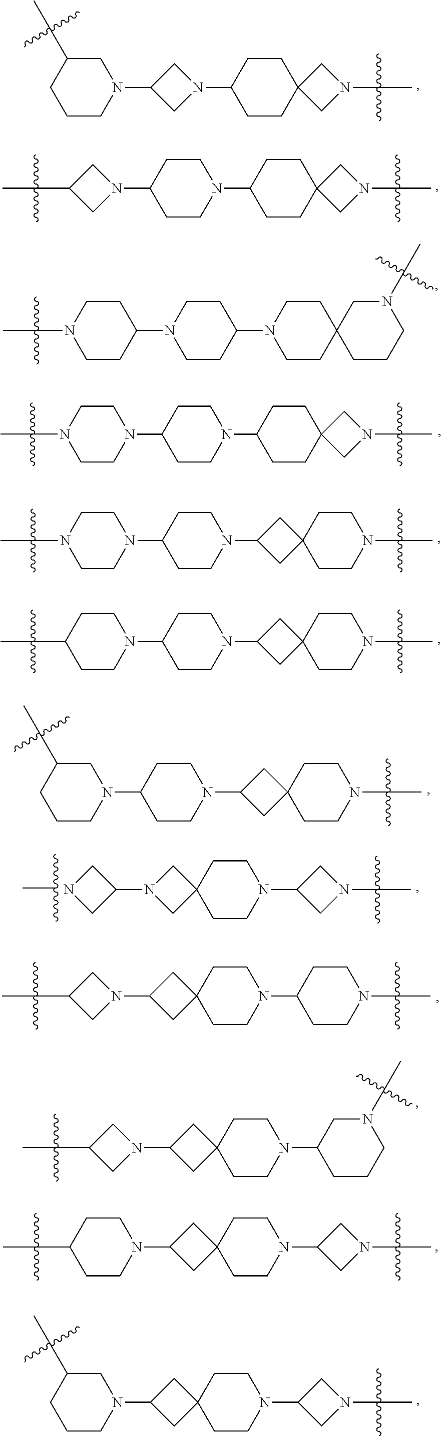

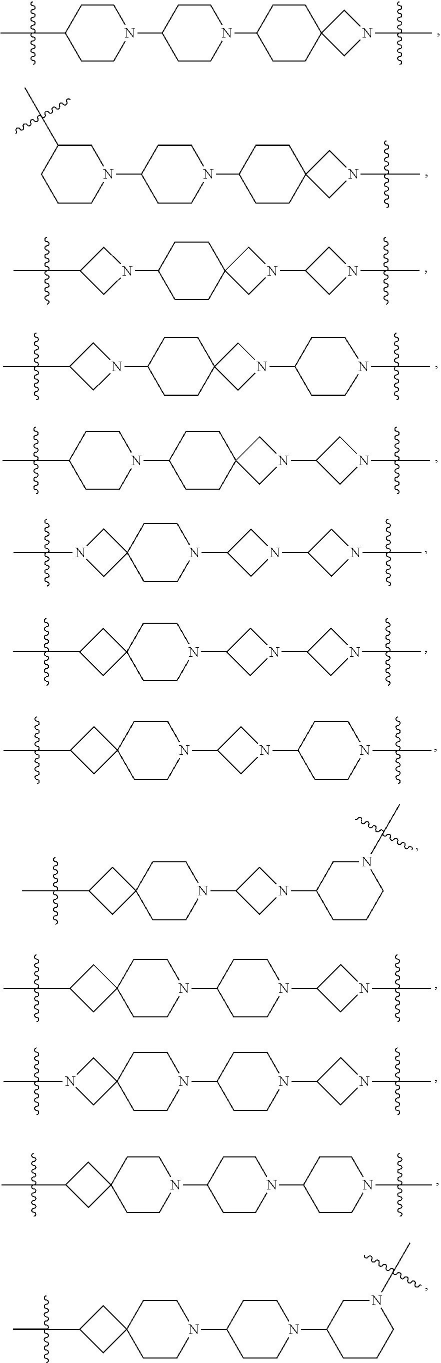

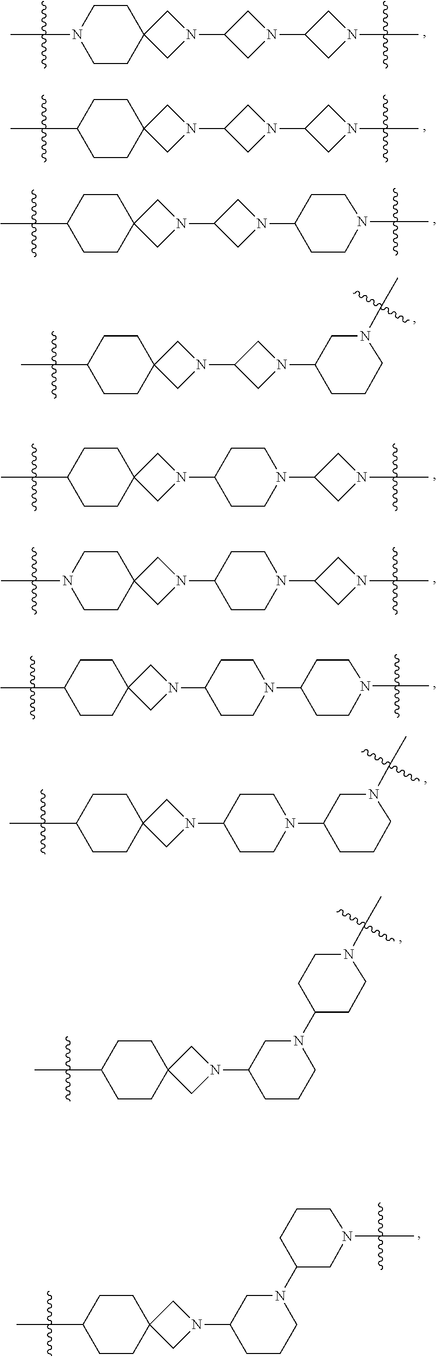

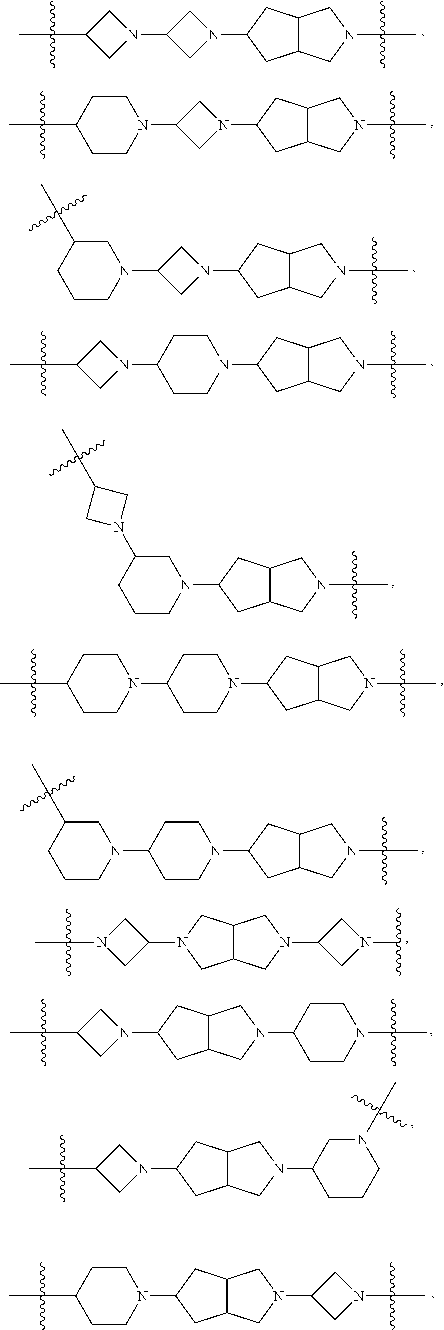

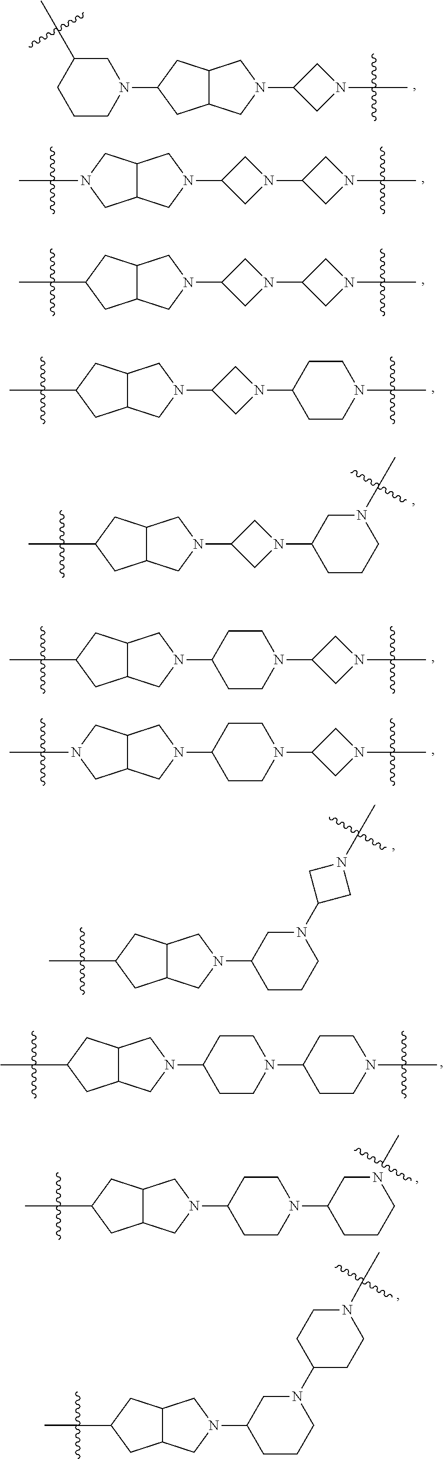

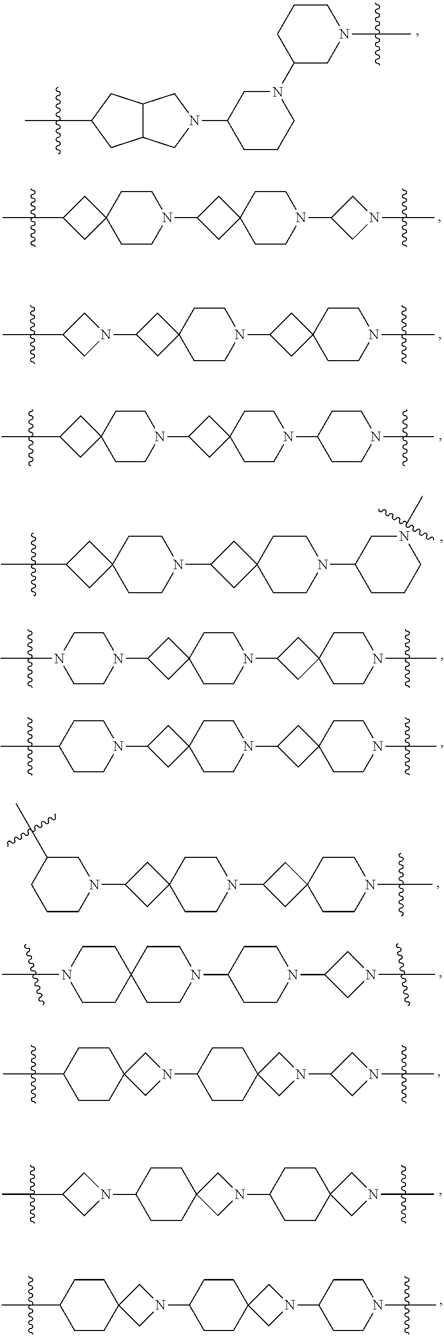

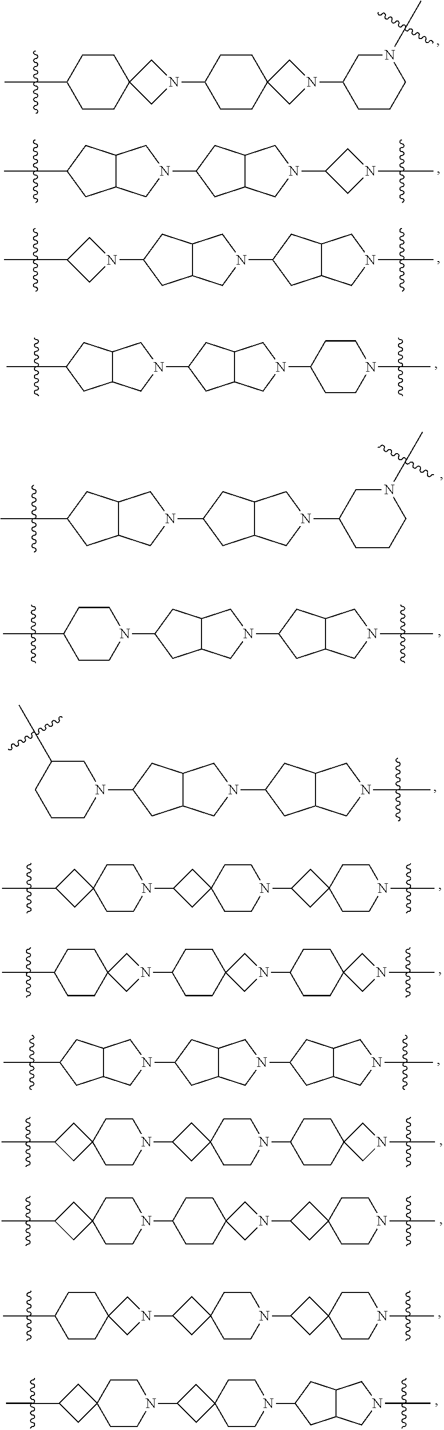

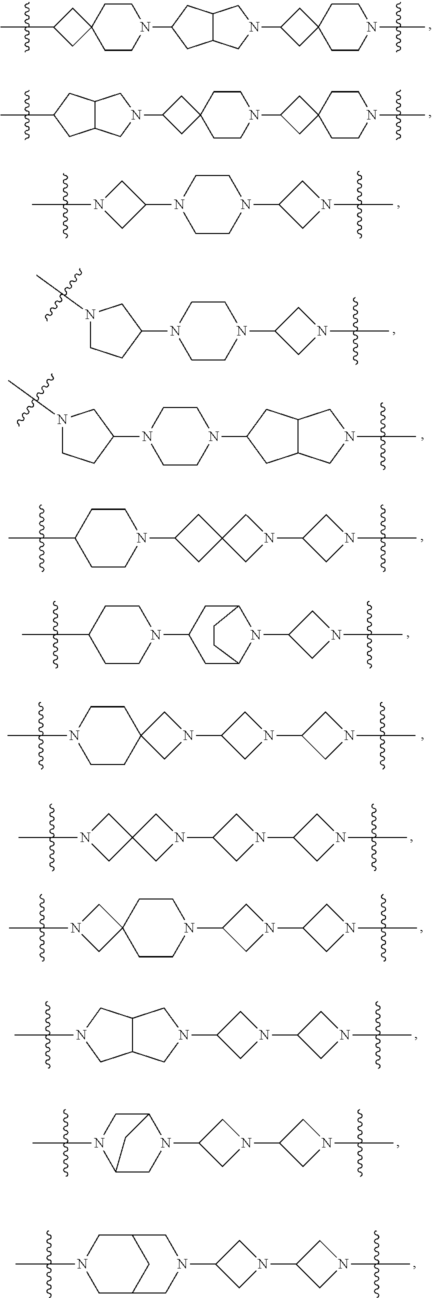

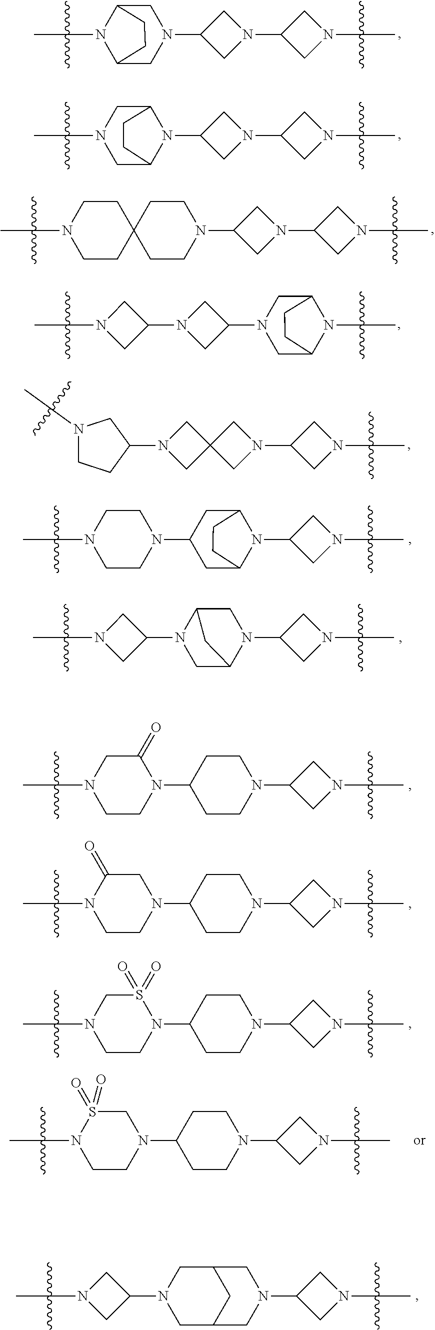

wherein the left side thereof is linked to B;
or L is selected from

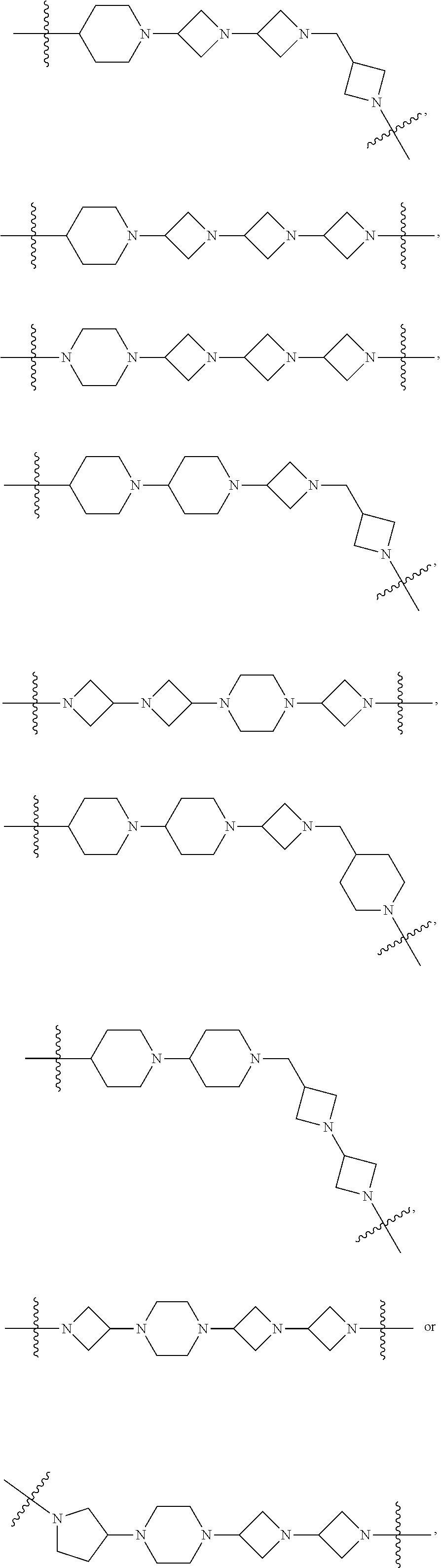

wherein the left side thereof is linked to B;
or L is selected from

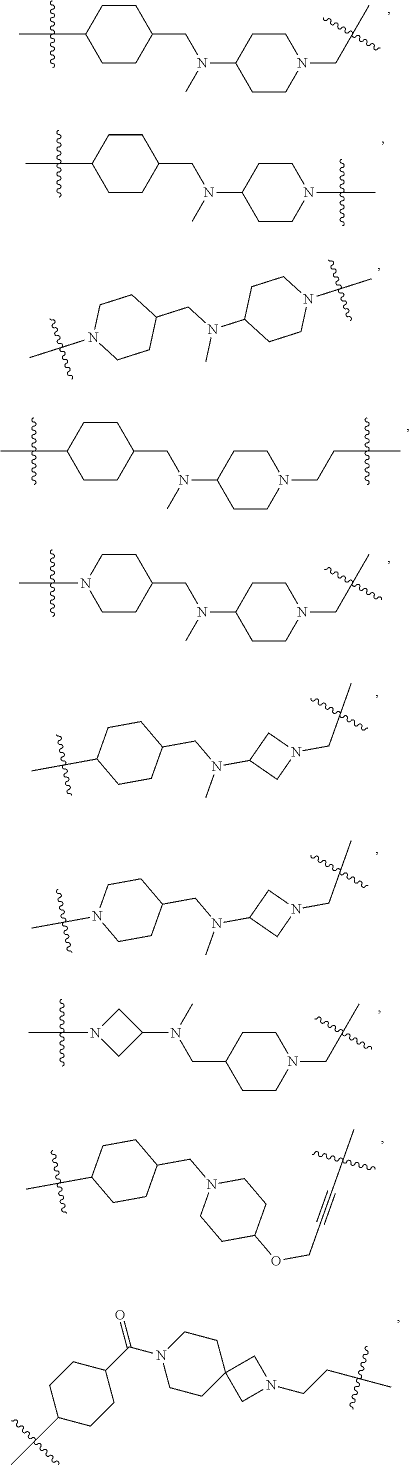

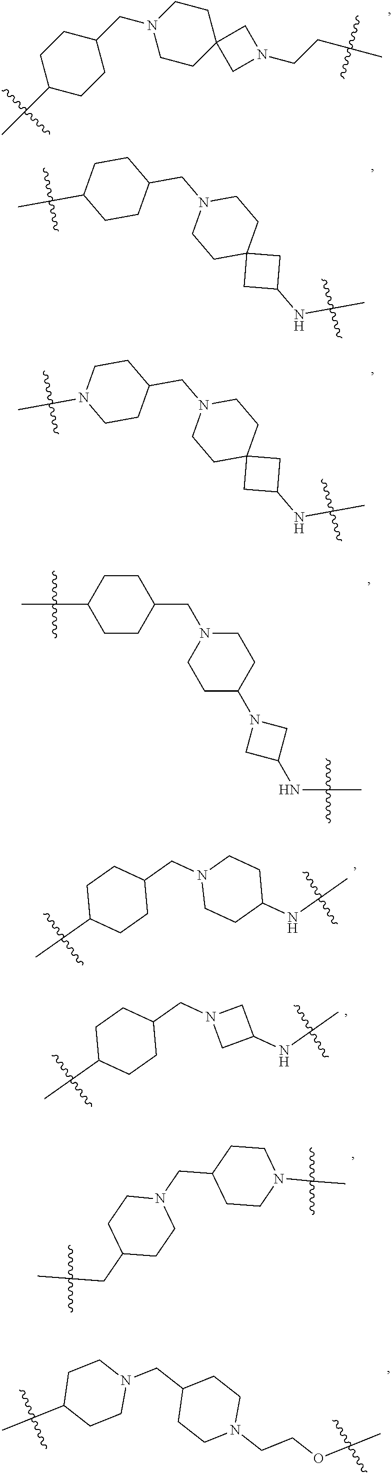

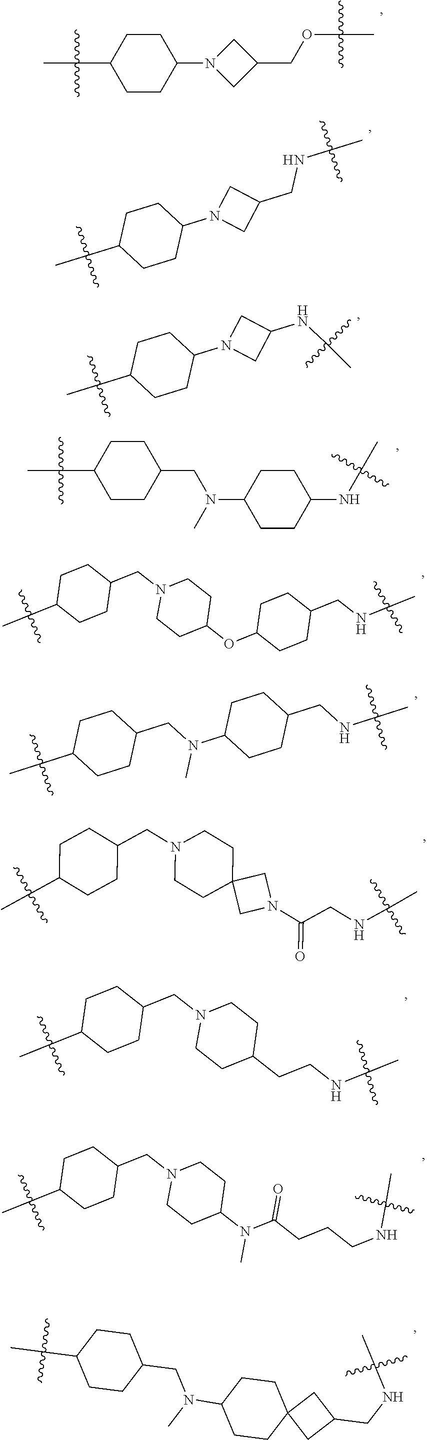

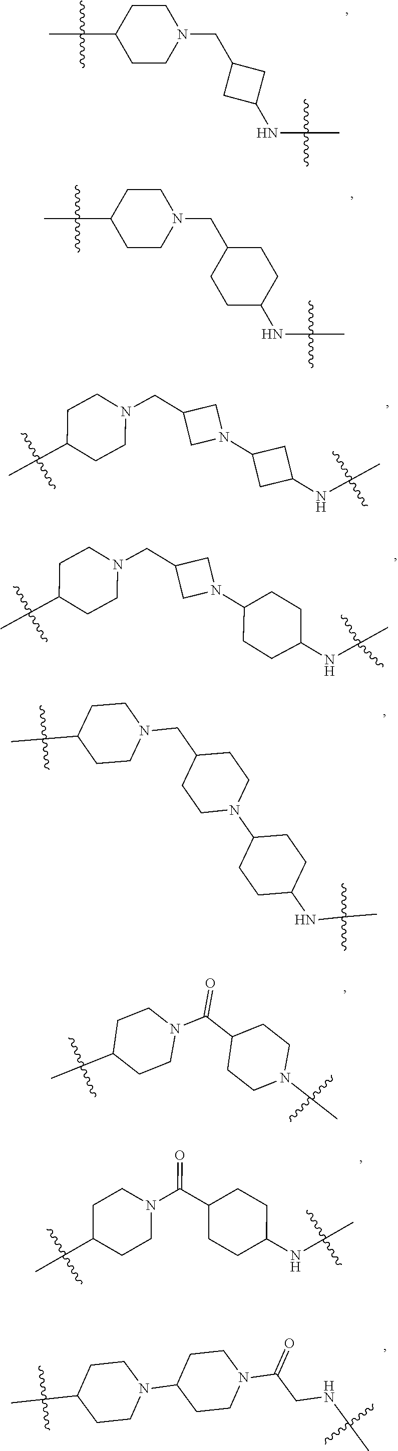

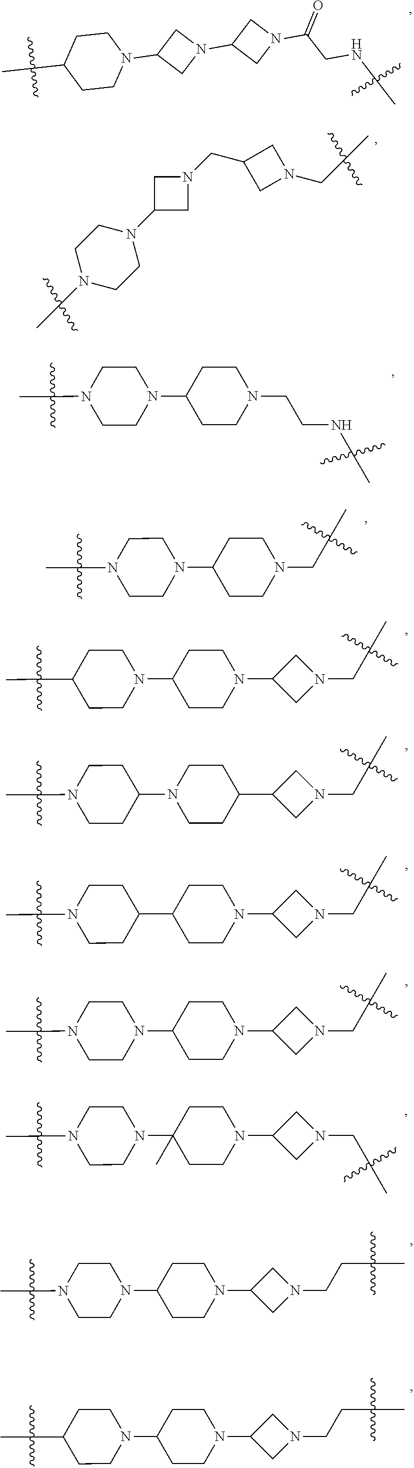

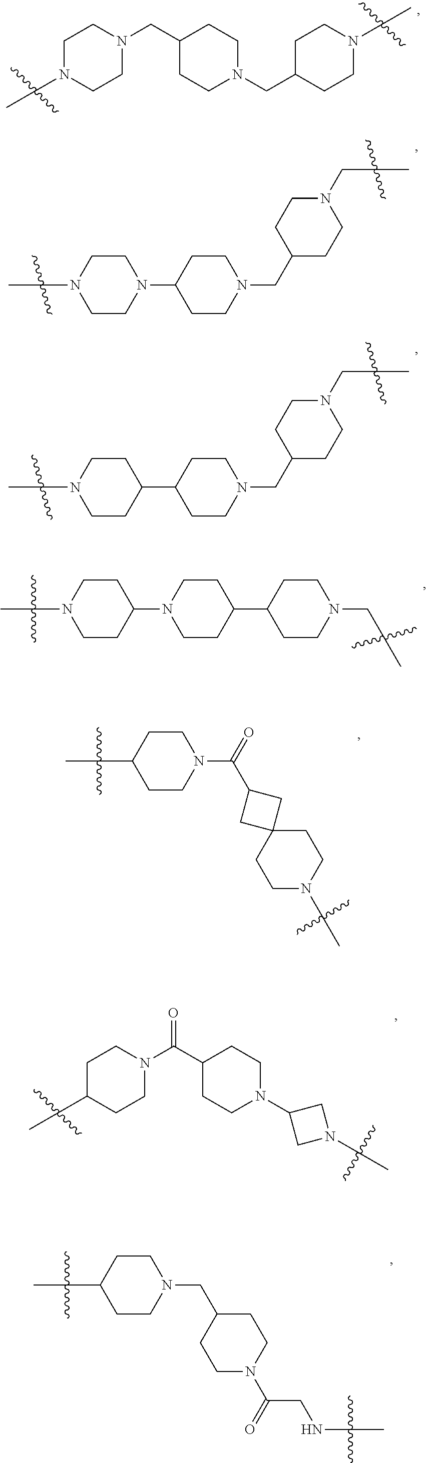

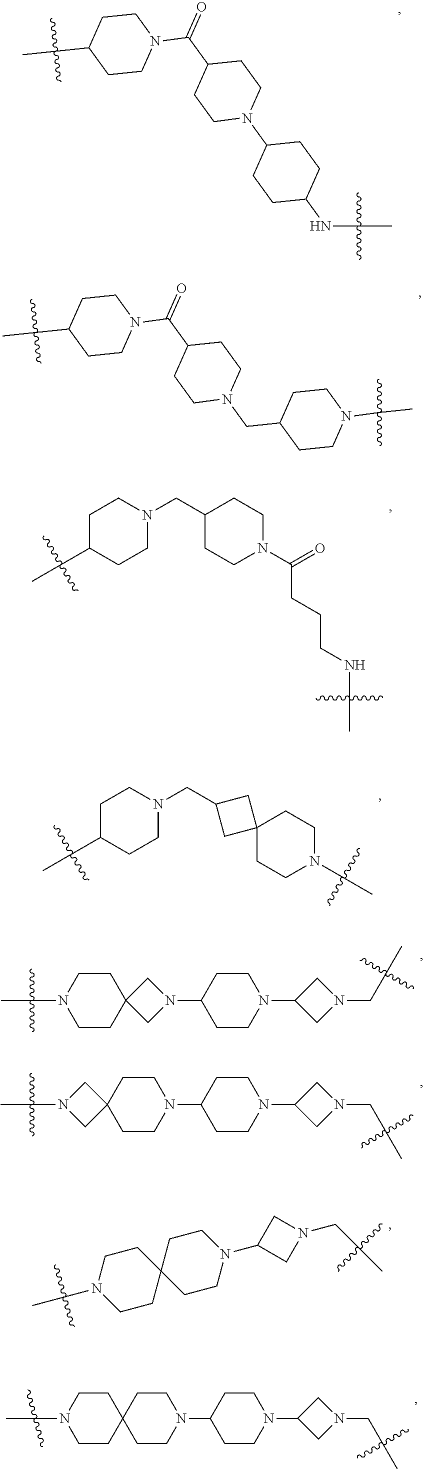

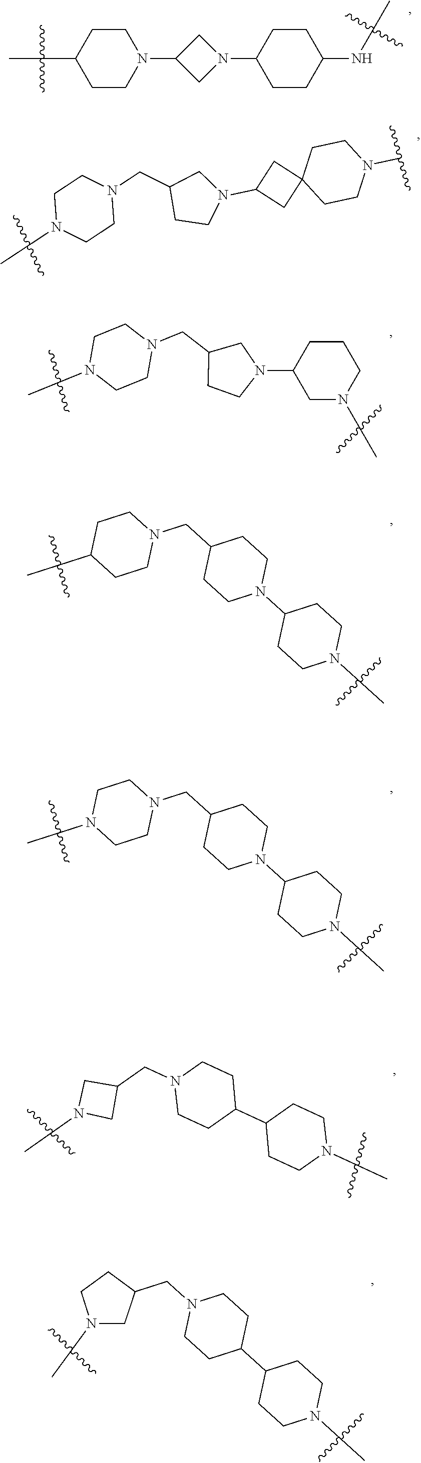

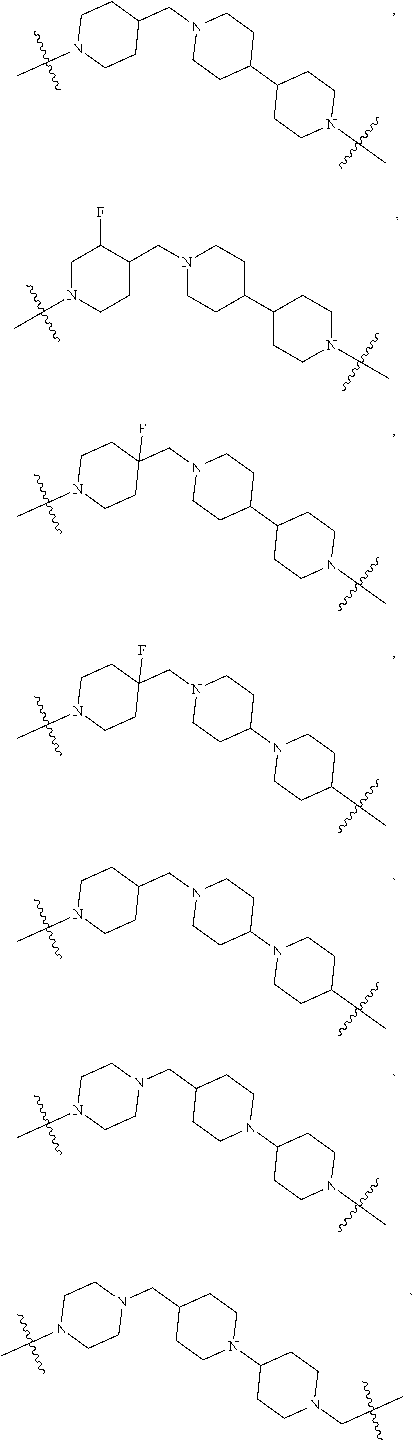

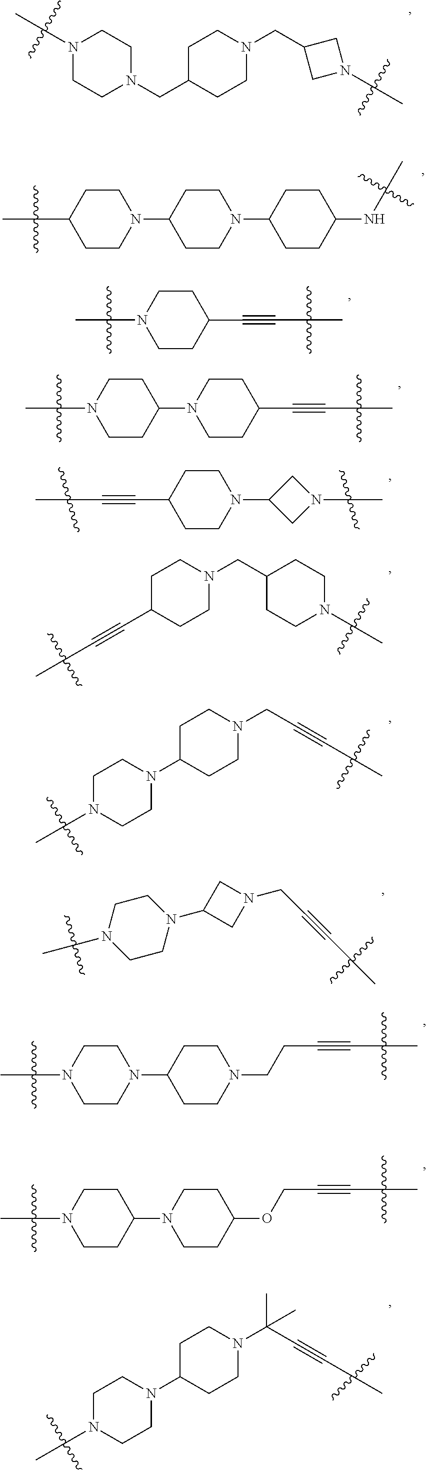

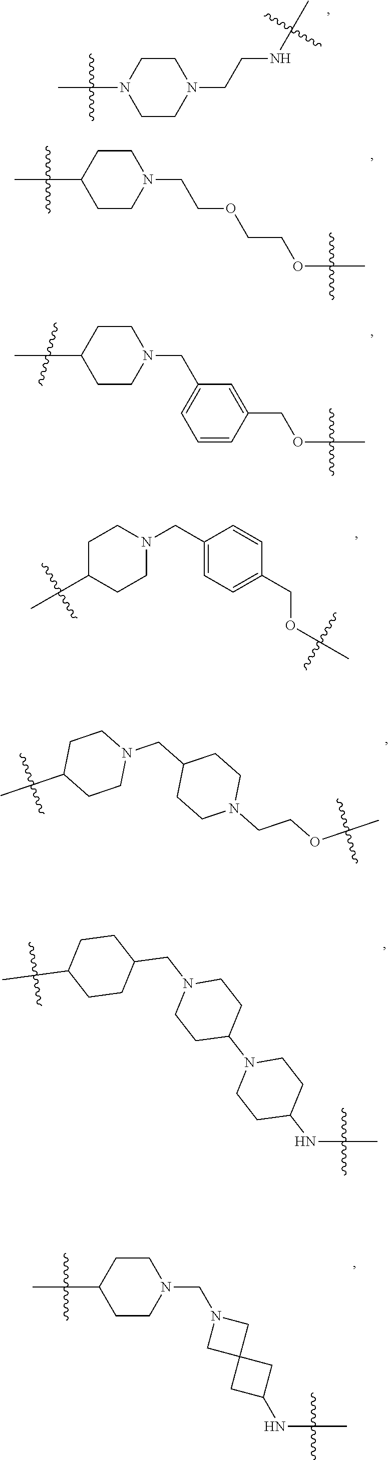

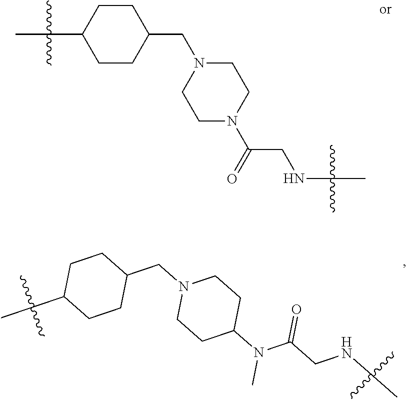 [SMILES: *NCC(=O)N1CCN(CC2CCC(*)CC2)CC1 ; *NCC(=O)N(C)C1CCN(CC2CCC(*)CC2)CC1]

wherein the left side thereof is linked to B;
the definitions of other groups are the same as those in any one of the first, second, third, fourth and fifth embodiments of the present invention.

As a seventh embodiment of the present invention, the above-mentioned compound as represented by general formula (I) or the stereoisomer, deuterated compound, solvate, prodrug, metabolite, pharmaceutically acceptable salt or co-crystal thereof, wherein
L is selected from -Cy1-Cy2-Cy3-Cy4-Ak1-Ak2-Ak3-Ak4-Ak5-, -Cy1-Ak1-Cy2-Ak2-Cy3-Ak3-Cy4-Ak4-Ak5-, -Ak1-Cy1-Ak2-Cy2-Ak3-Cy3-Ak4-Cy4-Ak5-, -Cy1-Ak1-Cy2-Ak2-Cy3-Cy4-Ak3-Ak4-Ak5-, -Cy1-Ak1-Cy2-Ak2-Ak3-Cy3-Cy4-Ak4-Ak5-, -Cy1-Ak1-Ak2-Ak3-Ak4-Ak5-Cy2-Cy3-Cy4-, -Cy1-Cy2-Ak1-Ak2-Ak3-Ak4-Ak5-Cy3-Cy4-, -Cy1-Cy2-Cy3-Ak1-Ak2-Ak3-Ak4-Ak5-Cy4-, -Cy1-Cy2-Cy3-Cy4-Ak1-Ak2-Ak3-Ak4-Ak5-, -Cy1-Ak1-Cy2-Cy3-Cy4-Ak2-Ak3-Ak4-Ak5-, -Cy1-Cy2-Ak1-Cy3-Cy4-Ak2-Ak3-Ak4-Ak5-, -Cy1-Cy2-Cy3-Ak1-Cy4-Ak2-Ak3-Ak4-Ak5-, -Cy1-Ak1-Ak2-Cy2-Cy3-Cy4-Ak3-Ak4-Ak5-, -Cy1-Cy2-Ak1-Ak2-Cy3-Cy4-Ak3-Ak4-Ak5-, -Cy1-Cy2-Cy3-Ak1-Ak2-Cy4-Ak3-Ak4-Ak5-, -Cy1-Ak1-Ak2-Ak3-Cy2-Cy3-Cy4-Ak4-Ak5-, -Cy1-Cy2-Ak1-Ak2-Ak3-Cy3-Cy4-Ak4-Ak5-, -Cy1-Cy2-Cy3-Ak1-Ak2-Ak3-Cy4-Ak4-Ak5-, -Cy1-Ak1-Ak2-Ak3-Ak4-Cy2-Cy3-Cy4-Ak5-, -Cy1-Cy2-Ak1-Ak2-Ak3-Ak4-Cy3-Cy4-Ak5-, -Cy1-Cy2-Cy3-Ak1-Ak2-Ak3-Ak4-Cy4-Ak5-, -Ak1-Ak2-Ak3-Ak4-Ak5-Cy1-Cy2-Cy3-Cy4-, -Ak1-Cy1-Cy2-Cy3-Cy4-Ak2-Ak3-Ak4-Ak5-, -Ak1-Ak2-Cy1-Cy2-Cy3-Cy4-Ak3-Ak4-Ak5-, -Ak1-Ak2-Ak3-Cy1-Cy2-Cy3-Cy4-Ak4-Ak5-, -Ak1-Ak2-Ak3-Ak4-Cy1-Cy2-Cy3-Cy4-Ak5-, -Ak1-Cy1-Ak2-Ak3-Ak4-Ak5-Cy2-Cy3-Cy4-, -Ak1-Cy1-Cy2-Ak2-Ak3-Ak4-Ak5-Cy3-Cy4-, -Ak1-Cy1-Cy2-Cy3-Ak2-Ak3-Ak4-Ak5-Cy4-, -Ak1-Ak2-Cy1-Ak3-Ak4-Ak5-Cy2-Cy3-Cy4-, -Ak1-Ak2-Cy1-Cy2-Ak3-Ak4-Ak5-Cy3-Cy4-, -Ak1-Ak2-Cy1-Cy2-Cy3-Ak3-Ak4-Ak5-Cy4-, -Ak1-Ak2-Ak3-Cy1-Ak4-Ak5-Cy2-Cy3-Cy4-, -Ak1-Ak2-Ak3-Cy1-Cy2-Ak4-Ak5-Cy3-Cy4-, -Ak1-Ak2-Ak3-Cy1-Cy2-Cy3-Ak4-Ak5-Cy4-, -Ak1-Ak2-Ak3-Ak4-Cy1-Ak5-Cy2-Cy3-Cy4-, -Ak1-Ak2-Ak3-Ak4-Cy1-Cy2-Ak5-Cy3-Cy4-, or -Ak1-Ak2-Ak3-Ak4-Cy1-Cy2-Cy3-Ak5-Cy4-;
Ak1, Ak2, Ak3, Ak4, and Ak5 are each independently selected from —(CH₂)_{q}—, —(CH₂)—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}, —(C≡C)_{q}— or a bond;
each q is independently selected from 0, 1, 2, 3 or 4;
each R^{L} is independently selected from H or C₁₋₆ alkyl;
the definitions of Cy1, Cy2, Cy3 and Cy4 are the same as those in the first embodiment of the present invention.

As an eighth embodiment of the present invention, the above-mentioned compound as represented by general formula (I) or the stereoisomer, deuterated compound, solvate, prodrug, metabolite, pharmaceutically acceptable salt or co-crystal thereof, wherein
the definition of Ak1, Ak2, Ak3, Ak4, Ak5, q, R^{L}, Cy1, Cy2, Cy3, Cy4, K, R^{b1}, R^{b2}, R^{b3}, R^{w1}, R^{w2}, R^{w3}, r or t is the same as that in any one of the first or third embodiment of the present invention.

As a ninth embodiment of the present invention, the above-mentioned compound as represented by general formula (I) or the stereoisomer, deuterated compound, solvate, prodrug, metabolite, pharmaceutically acceptable salt or co-crystal thereof, wherein
the definition of Ak1, Ak2, Ak3, Ak4, Ak5, q, r, t, R^{L}, Cy1, Cy2, Cy3, Cy4, K, R^{b1}, R^{b2}, R^{b3}, R^{w1}, R^{w2}, R^{w3}, B or K is the same as that in any one of the first, third or fourth embodiment of the present invention.

As a tenth embodiment of the present invention, the above-mentioned compound as represented by general formula (I) or the stereoisomer, deuterated compound, solvate, prodrug, metabolite, pharmaceutically acceptable salt or co-crystal thereof, wherein
the definition of Ak1, Ak2, Ak3, Ak4, Ak5, q, r, t, R^{L}, Cy1, Cy2, Cy3, Cy4, K, R^{b1}, R^{b2}, R^{b3}, R^{w1}, R^{w2}, R^{w3}, B or K is the same as that in any one of the first, third, fourth or fifth embodiment of the present invention.

As an eleventh embodiment of the present invention, the above-mentioned compound as represented by general formula (I) or the stereoisomer, deuterated compound, solvate, prodrug, metabolite, pharmaceutically acceptable salt or co-crystal thereof, wherein
L is selected from -Cy1-, -Cy1-Ak1-, -Cy1-Ak1-Ak2-, -Cy1-Ak1-Ak2-Ak3-, -Cy1-Ak1-Ak2-Ak3-Ak4-, -Cy1-Cy2-, -Cy1-Ak1-Cy2-, -Cy1-Cy2-Ak2-, -Cy1-Ak1-Cy2-Ak2-, -Cy1-Ak1-Cy2-Ak2-Ak3-, -Cy1-Ak1-Cy2-Ak2-Ak3-Ak4-, -Cy1-Cy2-Ak2-Ak3-, -Cy1-Cy2-Ak2-Ak3-Ak4-, -Cy1-Ak1-Cy2-Ak2-Ak3-Ak4-, -Cy1-Ak1-Ak2-Cy3-, -Cy1-Ak1-Ak2-Cy3-Ak3-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Ak1-Cy2-Cy3-Ak3-, -Cy1-Cy2-Ak2-Cy3-Ak3-, -Cy1-Ak1-Cy2-Ak2-Cy3-, -Cy1-Ak1-Cy2-Ak2-Cy3-Ak3-, -Cy1-Cy2-Cy3-Ak3-Ak4-, -Cy1-Cy2-Cy3-Cy4-, -Cy1-Ak1-Cy2-Cy3-Cy4-, -Cy1-Cy2-Ak2-Cy3-Cy4-, -Cy1-Cy2-Cy3-Ak3-Cy4-, -Cy1-Cy2-Cy3-Cy4-Ak4-, -Cy1-Ak1-Cy2-Ak2-Cy3-Ak3-Cy4-, -Cy1-Ak1-Cy2-Ak2-Cy3-Cy4-, -Ak1-Ak2-Cy3-Cy4-, -Ak1-Cy2-Ak2-Cy3-, -Ak1-Cy2-Ak2-, -Ak1-Ak2-Ak3-Ak4-, -Ak1-Ak2-Ak3-, -Ak1-Ak2-, -Ak1-Ak2-Ak3-Ak4-Ak5-, -Cy1-Cy2-Cy3-Ak3-Ak4-Ak5-, -Cy1-Cy2-Ak2-Cy3-Ak3-Ak4-Ak5-, -Cy1-Ak1-Cy2-Ak2-Ak3-Ak4-Ak5-, -Cy1-Cy2-Cy3-Cy4-Ak4-Ak5-, -Cy1-Ak1-Ak2-Ak3-Ak4-Ak5-, -Ak1-Cy2-Ak2-Ak3-Ak4-Ak5-, -Ak1-Cy2-Ak2-Ak3-Ak4-, or -Ak1-Cy2-Ak2-Ak3-;
Ak1, Ak2, Ak3, Ak4, and Ak5 are each independently selected from —O—, —OCH₂—, —CH₂O—, —OCH₂CH₂—, —CH₂CH₂O—, —C≡C—, —CH₂—, —CH₂CH₂—, —CH₂CH₂CH₂—, —N(CH₃)—, —NH—, —CH₂N(CH₃)—, —CH₂NH—, —NHCH₂—, —CH₂CH₂N(CH₃)—, —CH₂CH₂NH—, —NHCH₂CH₂—, —C(═O)—, —C(═O)CH₂NH—, —CH₂C(═O)NH—, —C(═O)NH— or —NHC(═O)—;
the definitions of other groups are the same as those in any one of the first, third, fourth and fifth embodiments of the present invention.

As a twelfth embodiment of the present invention, the above-mentioned compound as represented by general formula (I) or the stereoisomer, deuterated compound, solvate, prodrug, metabolite, pharmaceutically acceptable salt or co-crystal thereof, wherein
L is selected from

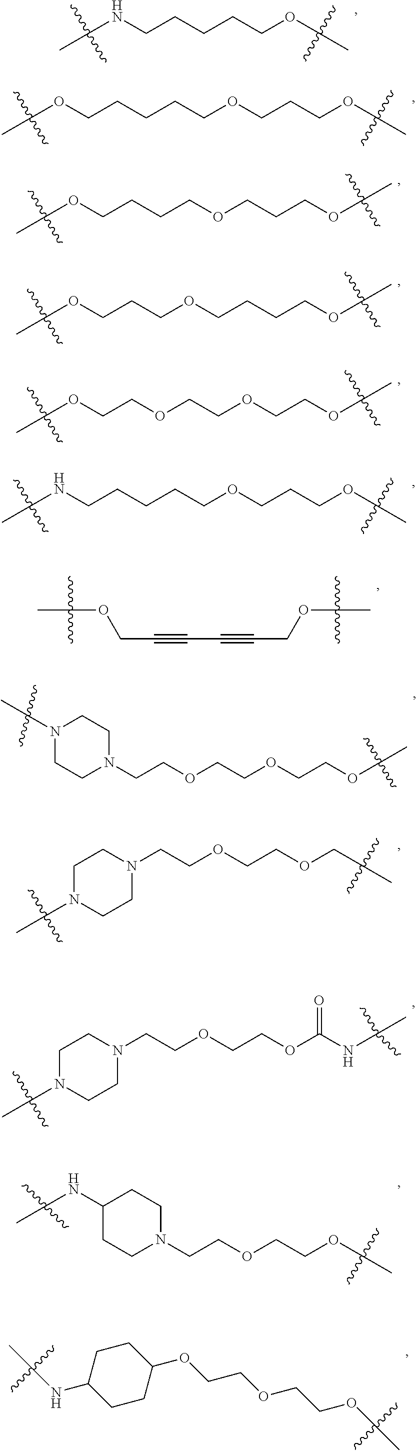

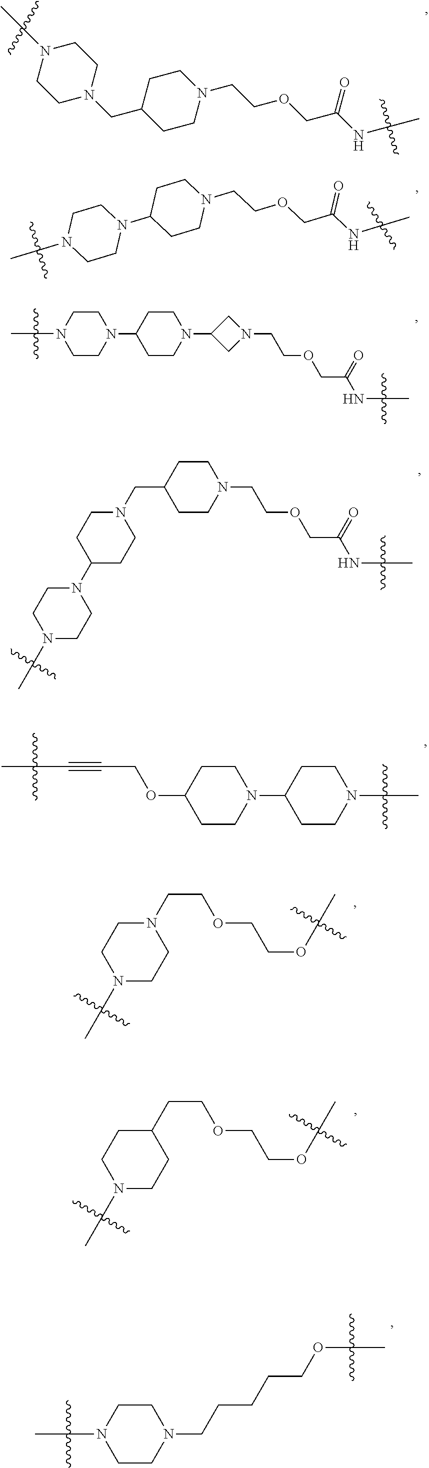

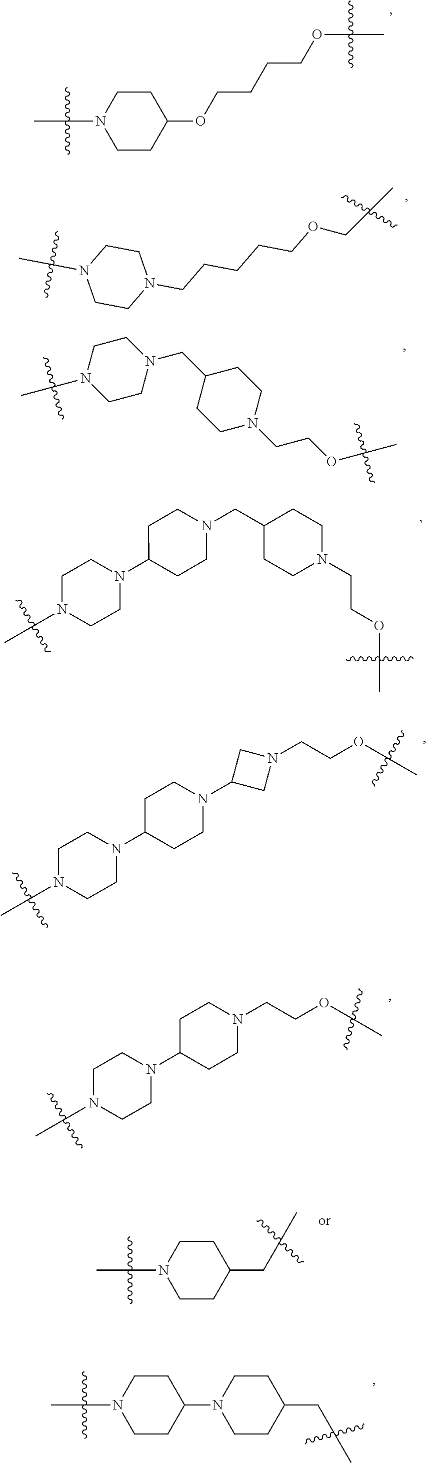

wherein the left side thereof is linked to B;
the definitions of other groups are the same as those in any one of the first, third, fourth, fifth and eleventh embodiments of the present invention.

As a thirteenth embodiment of the present invention, the above-mentioned compound as represented by general formula (I) or the stereoisomer, deuterated compound, solvate, prodrug, metabolite, pharmaceutically acceptable salt or co-crystal thereof, wherein
B is selected from

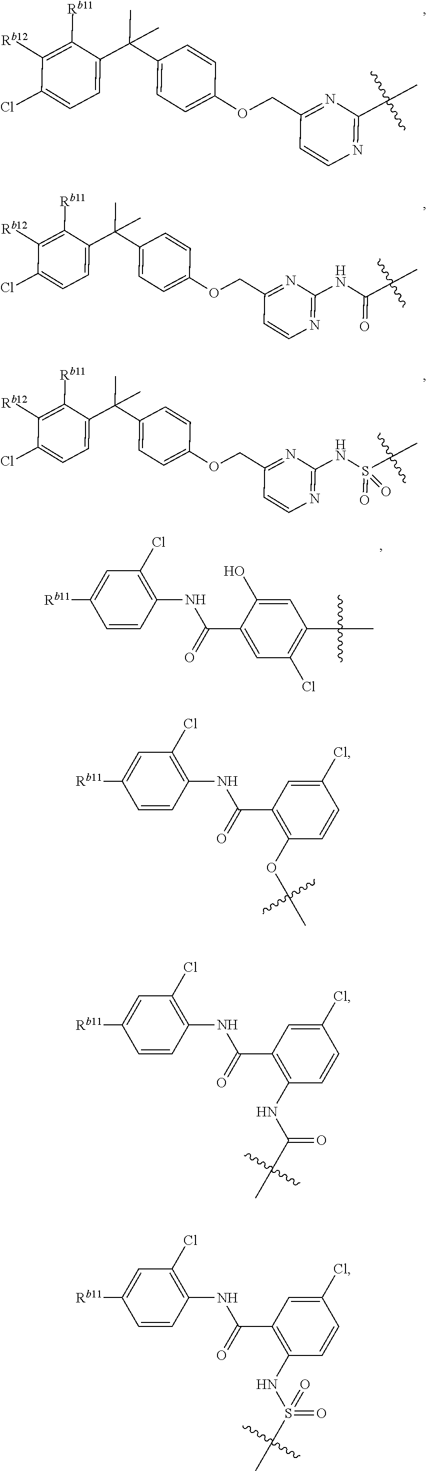

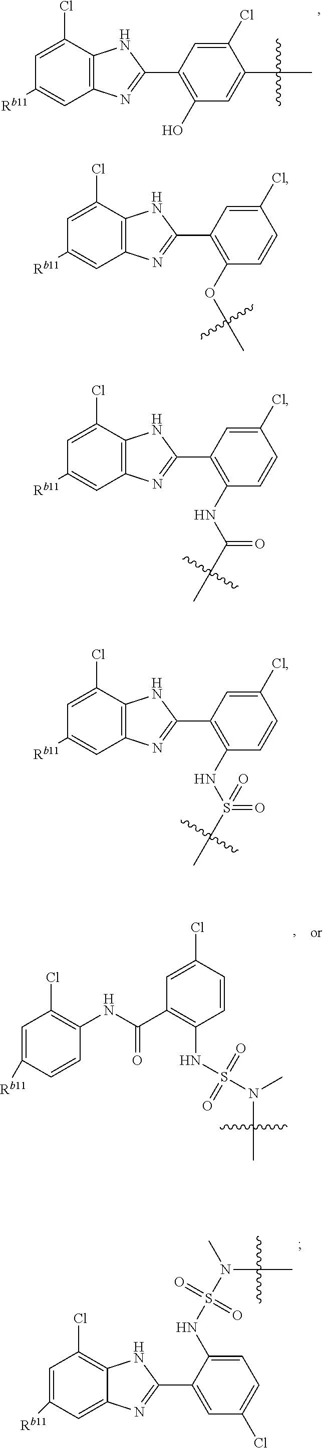

or B is selected from

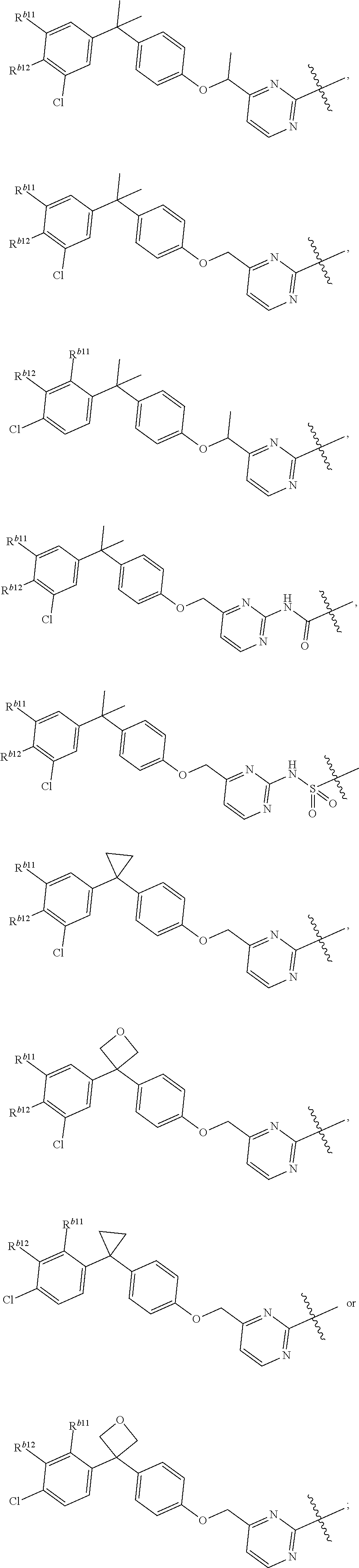

each R^{b11} is independently selected from F, Cl, Br, CF₃, CN, or NO₂;
each R^{b12} is independently selected from H, OCH₃, OCD₃, OCH₂CH₃, OCH₂CH₂Cl, or OCH₂CH₂CH₂Cl;
K is selected from

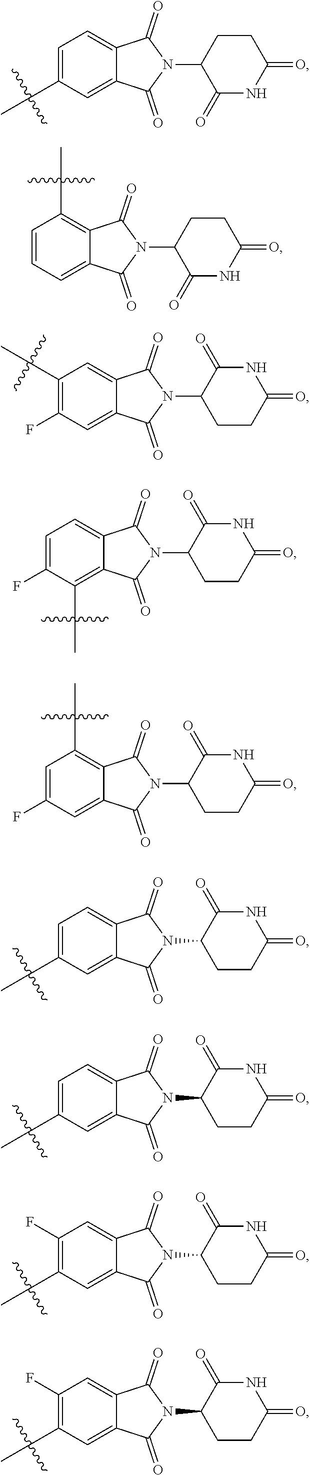

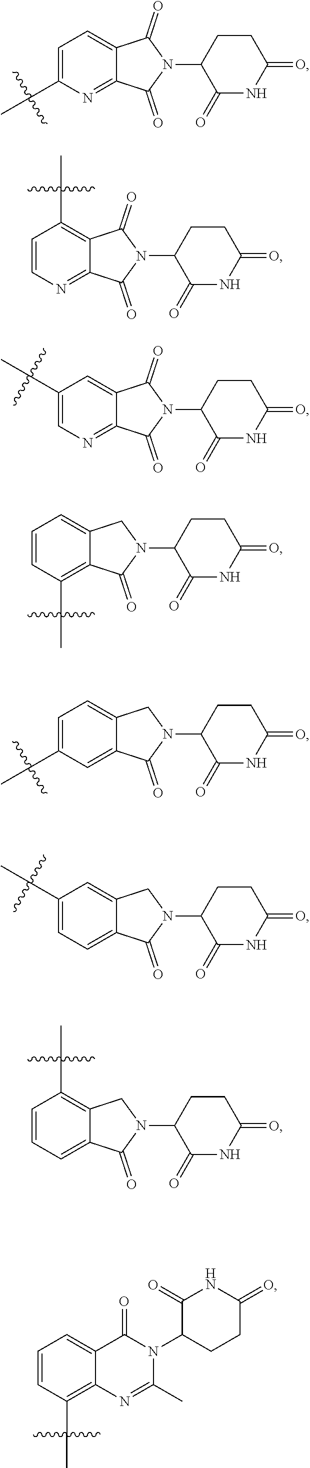

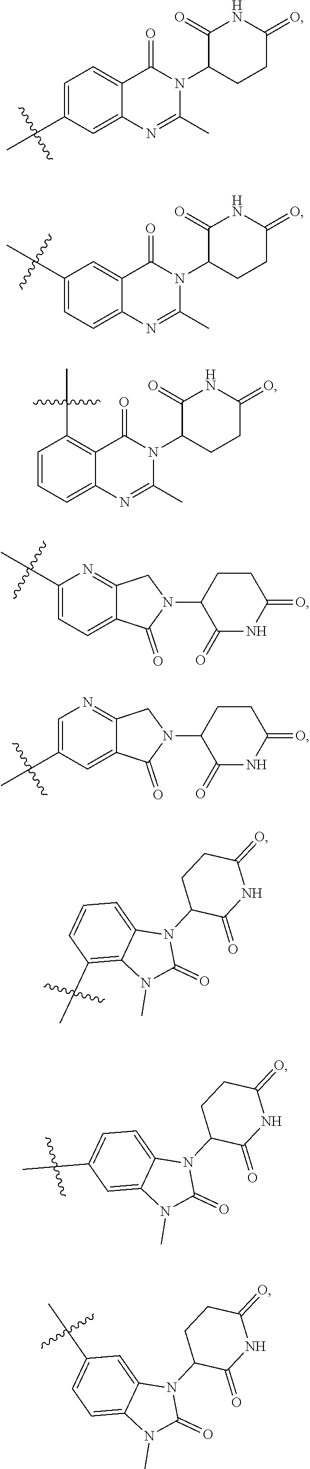

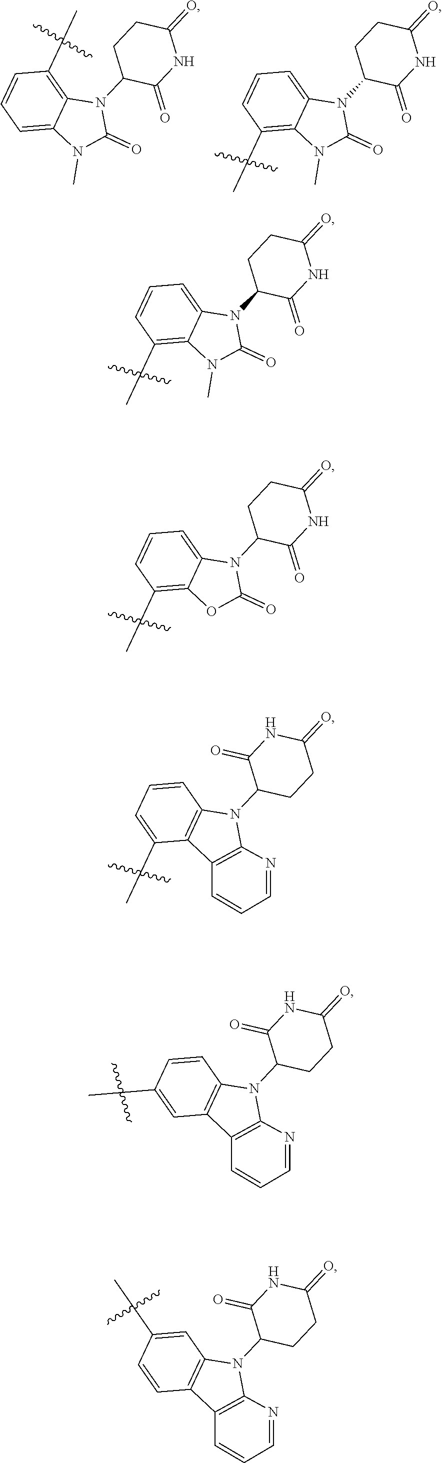

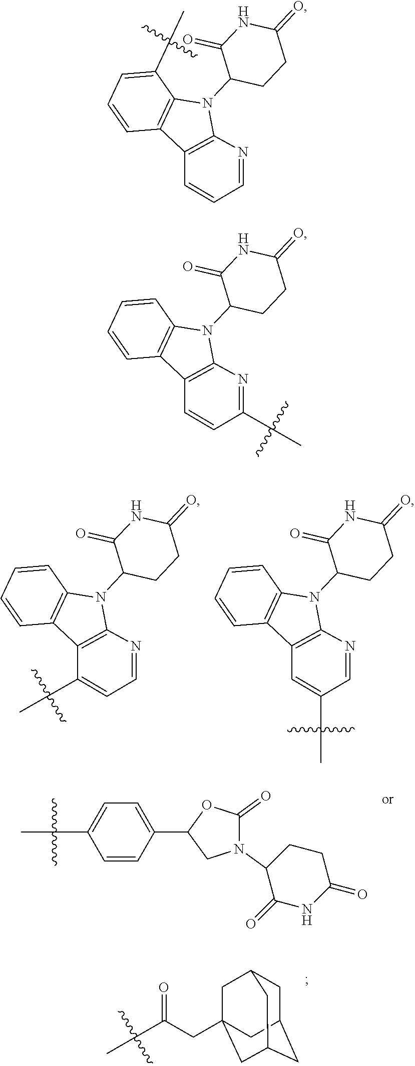

or K is selected from

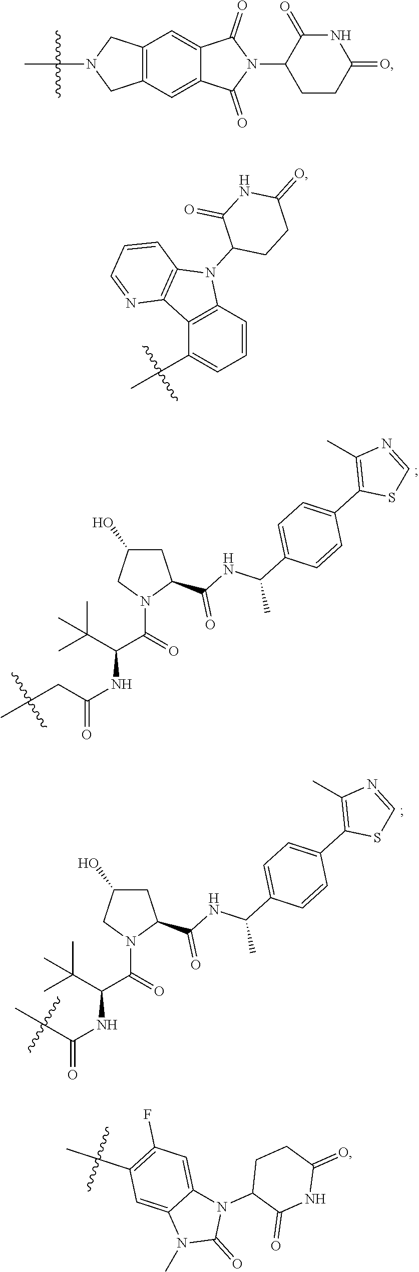 [SMILES: *N1Cc2cc3c(cc2C1)C(=O)N(C1CCC(=O)NC1=O)C3=O ; *c1cccc2c1c1ncccc1n2C1CCC(=O)NC1=O ; *CC(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C ; *c1cc2c(cc1F)n(C1CCC(=O)NC1=O)c(=O)n2C]

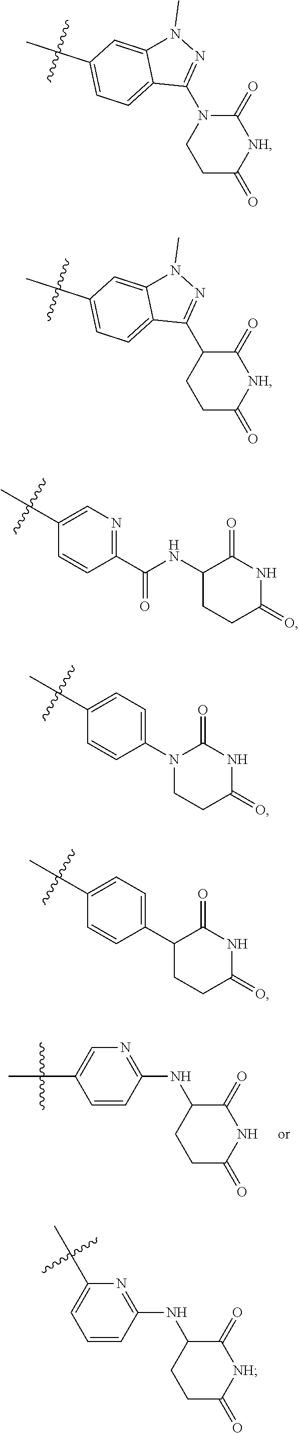

the definitions of other groups are the same as those in any one of the first, second, third, fourth, fifth, sixth, eleventh and twelfth embodiments of the present invention.

As a fourteenth embodiment of the present invention, the above-mentioned compound as represented by general formula (I) or the stereoisomer, deuterated compound, solvate, prodrug, metabolite, pharmaceutically acceptable salt or co-crystal thereof, wherein
B is selected from

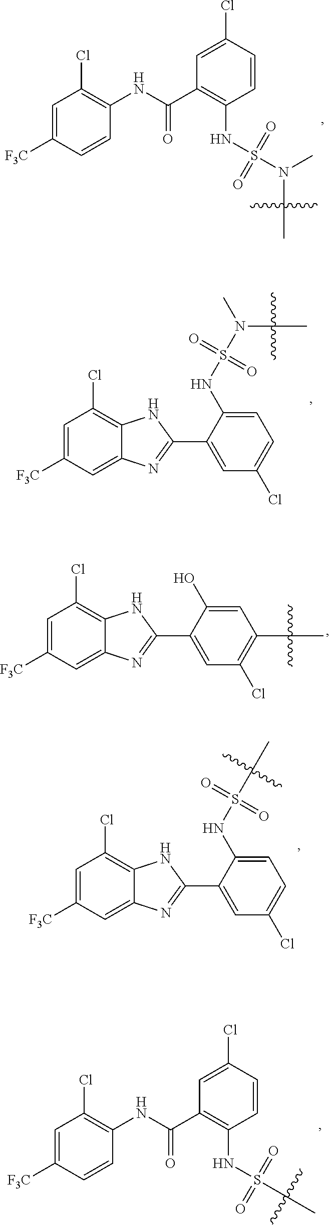 [SMILES: *N(C)S(=O)(=O)Nc1ccc(Cl)cc1C(=O)Nc1ccc(C(F)(F)F)cc1Cl ; *N(C)S(=O)(=O)Nc1ccc(Cl)cc1-c1nc2cc(C(F)(F)F)cc(Cl)c2[nH]1 ; *c1cc(O)c(-c2nc3cc(C(F)(F)F)cc(Cl)c3[nH]2)cc1Cl ; *S(=O)(=O)Nc1ccc(Cl)cc1-c1nc2cc(C(F)(F)F)cc(Cl)c2[nH]1 ; *S(=O)(=O)Nc1ccc(Cl)cc1C(=O)Nc1ccc(C(F)(F)F)cc1Cl]

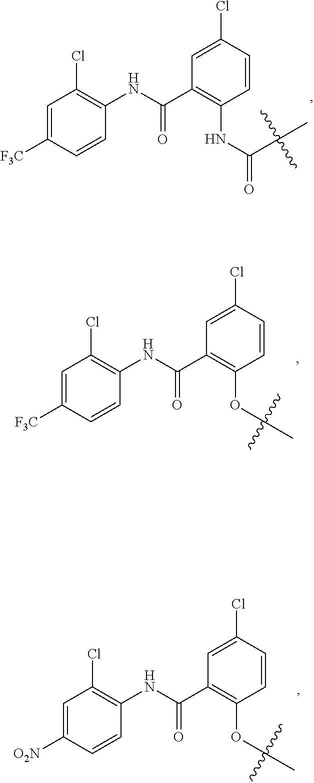 [SMILES: *C(=O)Nc1ccc(Cl)cc1C(=O)Nc1ccc(C(F)(F)F)cc1Cl ; *Oc1ccc(Cl)cc1C(=O)Nc1ccc(C(F)(F)F)cc1Cl ; *Oc1ccc(Cl)cc1C(=O)Nc1ccc([N+](=O)[O-])cc1Cl]

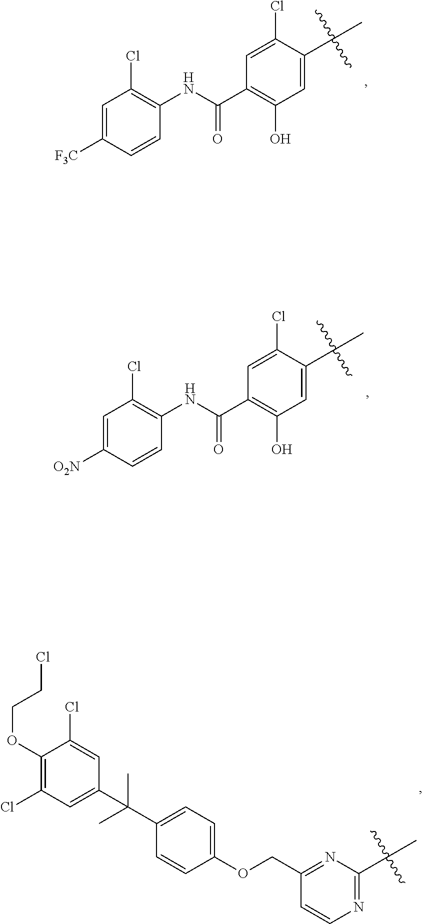 [SMILES: *c1cc(O)c(C(=O)Nc2ccc(C(F)(F)F)cc2Cl)cc1Cl ; *c1cc(O)c(C(=O)Nc2ccc([N+](=O)[O-])cc2Cl)cc1Cl ; *c1nccc(COc2ccc(C(C)(C)c3cc(Cl)c(OCCCl)c(Cl)c3)cc2)n1]

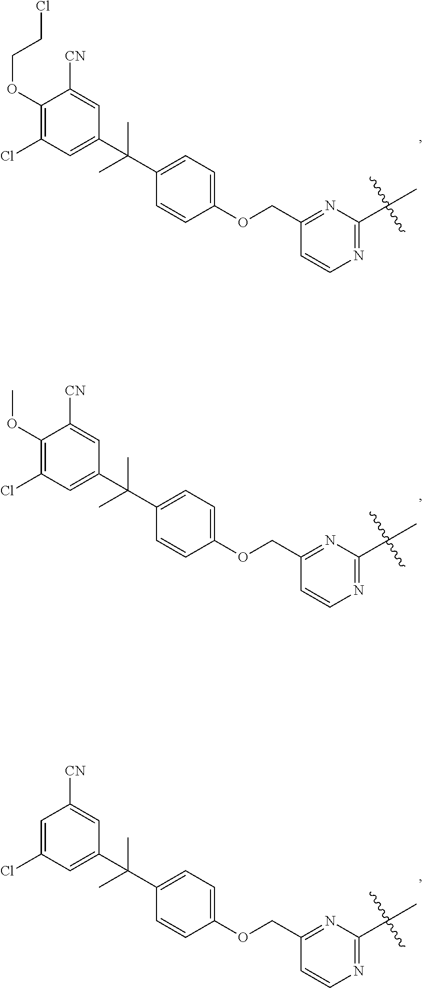 [SMILES: *c1nccc(COc2ccc(C(C)(C)c3cc(Cl)c(OCCCl)c(C#N)c3)cc2)n1 ; *c1nccc(COc2ccc(C(C)(C)c3cc(Cl)c(OC)c(C#N)c3)cc2)n1 ; *c1nccc(COc2ccc(C(C)(C)c3cc(Cl)cc(C#N)c3)cc2)n1]

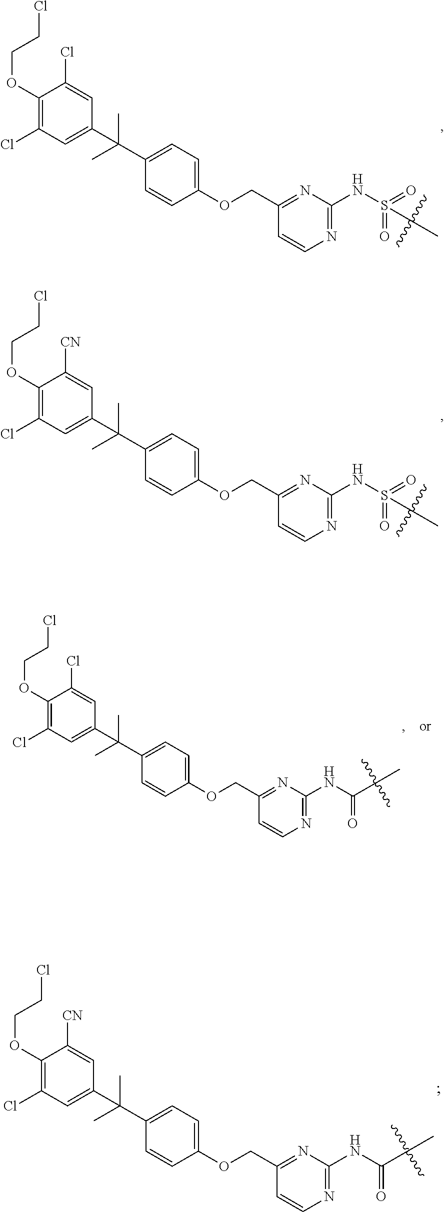 [SMILES: *S(=O)(=O)Nc1nccc(COc2ccc(C(C)(C)c3cc(Cl)c(OCCCl)c(Cl)c3)cc2)n1 ; *S(=O)(=O)Nc1nccc(COc2ccc(C(C)(C)c3cc(Cl)c(OCCCl)c(C#N)c3)cc2)n1 ; *C(=O)Nc1nccc(COc2ccc(C(C)(C)c3cc(Cl)c(OCCCl)c(Cl)c3)cc2)n1 ; *C(=O)Nc1nccc(COc2ccc(C(C)(C)c3cc(Cl)c(OCCCl)c(C#N)c3)cc2)n1]

or B is selected from

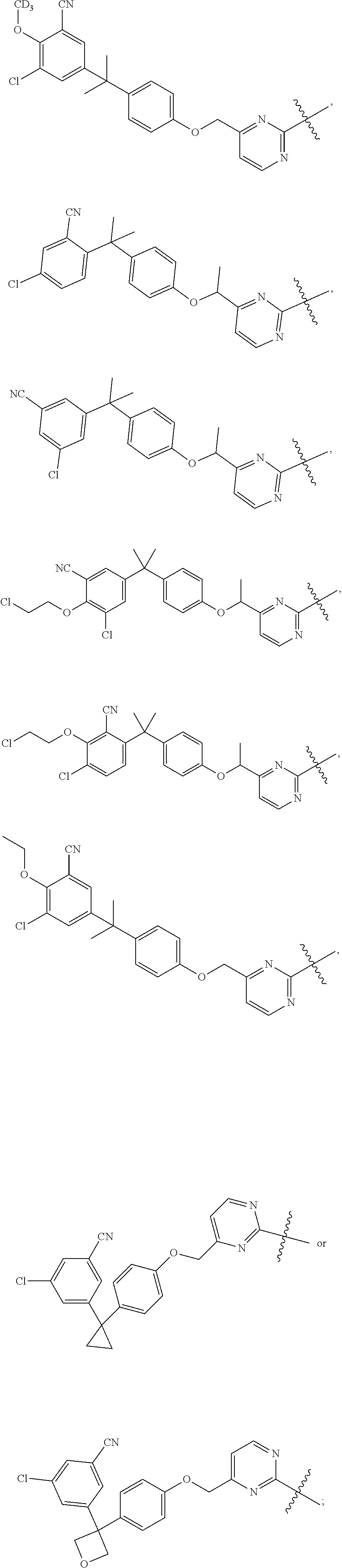

K is selected from

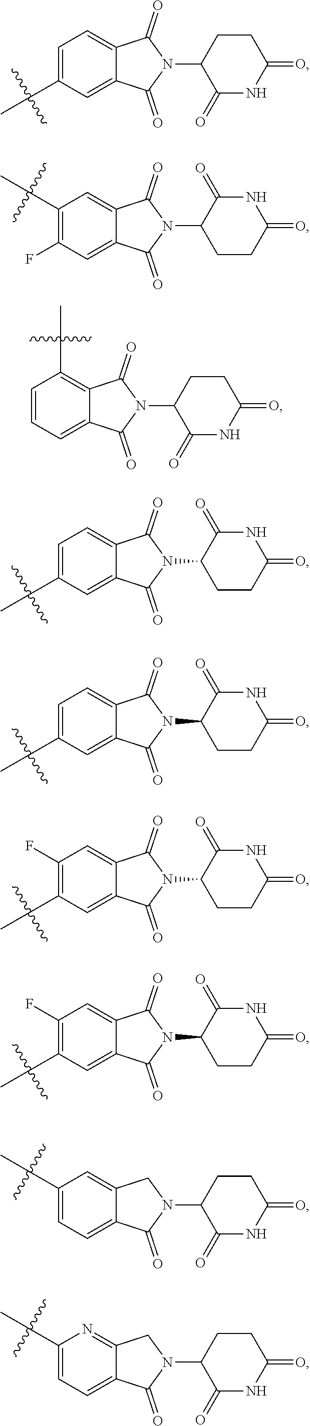

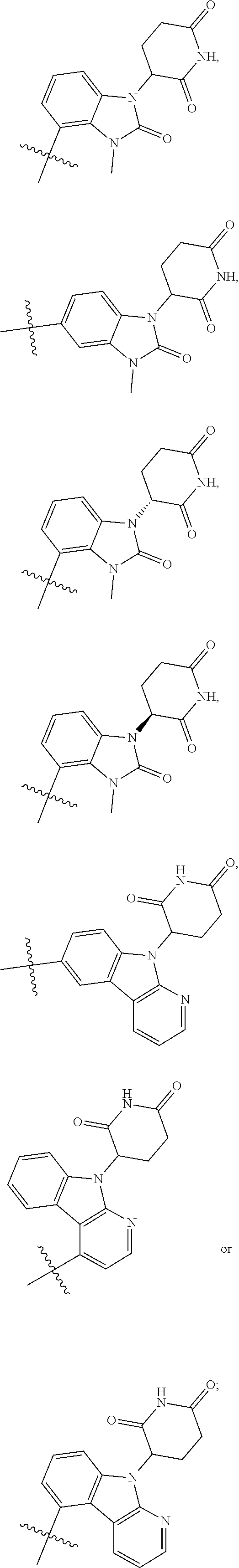

or K is selected from

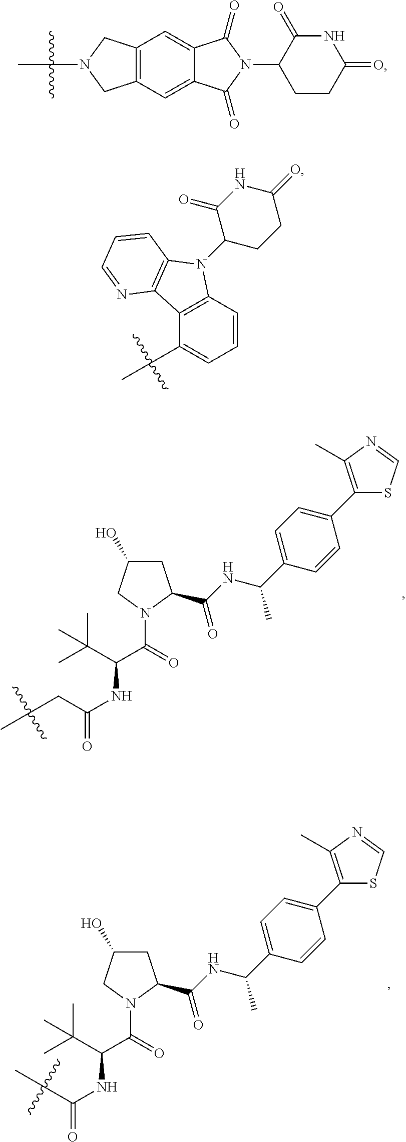 [SMILES: *N1Cc2cc3c(cc2C1)C(=O)N(C1CCC(=O)NC1=O)C3=O ; *c1cccc2c1c1ncccc1n2C1CCC(=O)NC1=O ; *CC(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

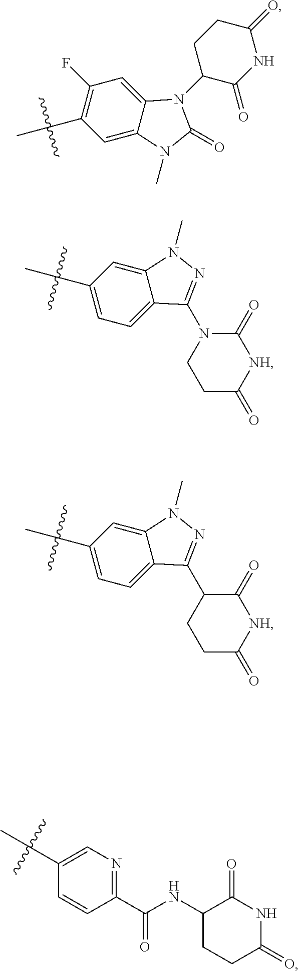

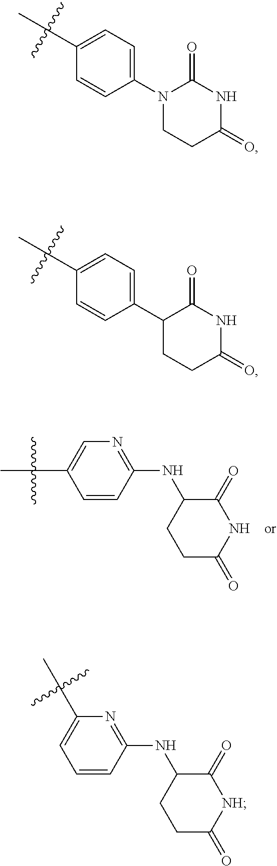 [SMILES: *c1ccc(N2CCC(=O)NC2=O)cc1 ; *c1ccc(C2CCC(=O)NC2=O)cc1 ; *c1ccc(NC2CCC(=O)NC2=O)nc1 ; *c1cccc(NC2CCC(=O)NC2=O)n1]

the definitions of other groups are the same as those in any one of the first, second, third, fourth, fifth, sixth, eleventh and twelfth embodiments of the present invention.

As a fifteenth embodiment of the present invention, the above-mentioned compound as represented by general formula (I) or the stereoisomer, deuterated compound, solvate, prodrug, metabolite, pharmaceutically acceptable salt or co-crystal thereof, wherein
each B is independently selected from one of the groups in Table B-1, preferably from one of the groups in Table B-2, wherein the right side thereof is linked to L;
each R^{b11} is independently selected from F, Cl, Br, CF₃, CN, or NO₂;
each R^{b12} is independently selected from H, OCH₃, OCD₃, OCH₂CH₃, or OCH₂CH₂Cl;
each L is independently selected from -Cy1-, -Cy1-Ak1-, -Cy1-Cy2-, -Cy1-Ak1-Cy2-, -Cy1-Cy2-Cy3-, -Cy1-Ak1-Cy2-Cy3-, -Cy1-Ak1-Cy2-Ak2-Cy3-, -Cy1-Cy2-Ak2-, -Cy1-Cy2-Ak2-Cy3-, -Cy1-Cy2-Cy3-Ak3-, or -Cy1-Cy2-Ak2-Cy3-Ak3-;
L is preferably selected from one of the groups in Table L-1, wherein the right side thereof is linked to K;
Ak1, Ak2, and Ak3 are each independently selected from —C≡C—, —CH₂—, —CH₂CH₂—, —N(CH₃)—, —NH—, —CH₂N(CH₃)— or —CH₂NH—;
Cy1, Cy2 and Cy3 are each independently selected from 4- to 7-membered mono-heterocyclic ring, 4- to 10-membered fused heterocyclic ring, 5- to 12-membered spiro-heterocyclic ring, wherein the mono-heterocyclic ring, fused heterocyclic ring or spiro-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring or spiro-heterocyclic ring contains 1 to 4 heteroatoms selected from O, S, or N (preferably, N);
each K is independently selected from one of the groups in Table K-1, wherein the left side thereof is linked to L.

As a sixteenth embodiment of the present invention, the above-mentioned compound as represented by general formula (I) or the stereoisomer, deuterated compound, solvate, prodrug, metabolite, pharmaceutically acceptable salt or co-crystal thereof, wherein
Cy1, Cy2, and Cy3 are each independently selected from one of the following groups:

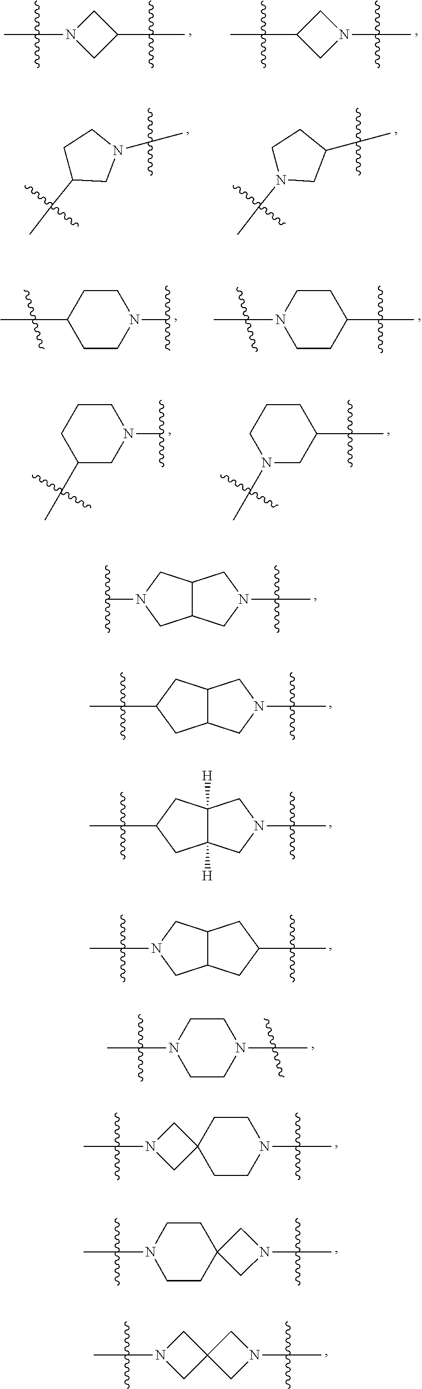

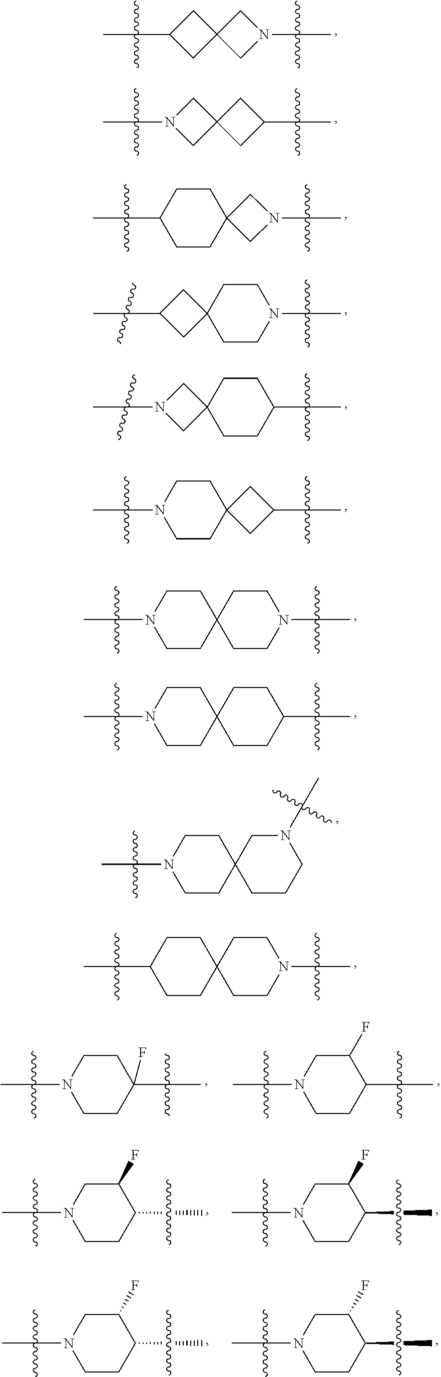

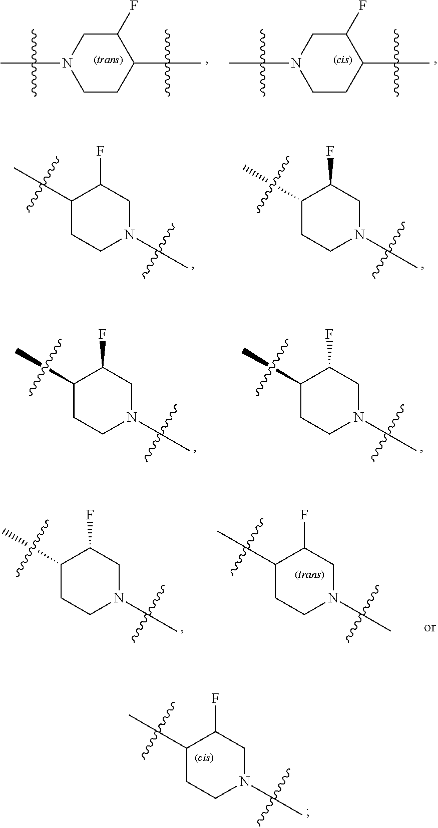

the definition of Ak1, Ak2, Ak3, R^{b11}, R^{b12}, L, B or K is the same as that in the fifteenth embodiment of the present invention.

As a seventeenth embodiment of the present invention, the above-mentioned compound as represented by general formula (I) or the stereoisomer, deuterated compound, solvate, prodrug, metabolite, pharmaceutically acceptable salt or co-crystal thereof, wherein
each B is independently selected from one of the groups in Table B-2, wherein the right side thereof is linked to L;
each L is independently selected from one of the groups in Table L-1, wherein the right side thereof is linked to K;
each K is independently selected from one of the groups in Table K-1, wherein the left side thereof is linked to L.

The present invention relates to a compound as described below or a stereoisomer, deuterated compound, solvate, prodrug, metabolite, pharmaceutically acceptable salt or co-crystal thereof, wherein the compound is of the structure selected from one of (Table D):

TABLE D

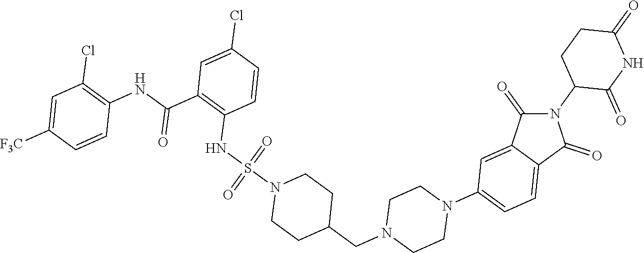 [SMILES: O=C1CCC(N2C(=O)c3ccc(N4CCN(CC5CCN(S(=O)(=O)Nc6ccc(Cl)cc6C(=O)Nc6ccc(C(F)(F)F)cc6Cl)CC5)CC4)cc3C2=O)C(=O)N1]

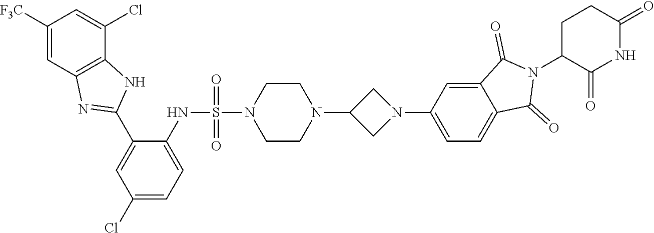 [SMILES: O=C1CCC(N2C(=O)c3ccc(N4CC(N5CCN(S(=O)(=O)Nc6ccc(Cl)cc6-c6nc7cc(C(F)(F)F)cc(Cl)c7[nH]6)CC5)C4)cc3C2=O)C(=O)N1]

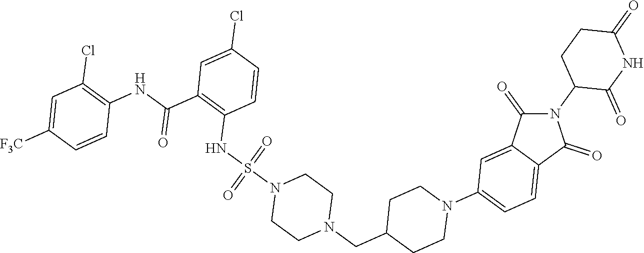 [SMILES: O=C1CCC(N2C(=O)c3ccc(N4CCC(CN5CCN(S(=O)(=O)Nc6ccc(Cl)cc6C(=O)Nc6ccc(C(F)(F)F)cc6Cl)CC5)CC4)cc3C2=O)C(=O)N1]

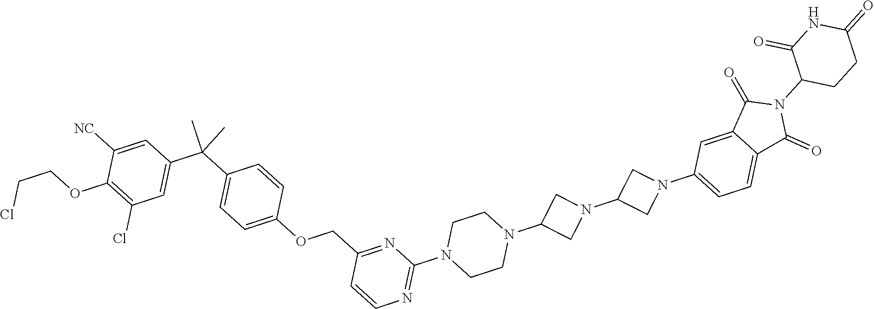 [SMILES: CC(C)(c1ccc(OCc2ccnc(N3CCN(C4CN(C5CN(c6ccc7c(c6)C(=O)N(C6CCC(=O)NC6=O)C7=O)C5)C4)CC3)n2)cc1)c1cc(Cl)c(OCCCl)c(C#N)c1]

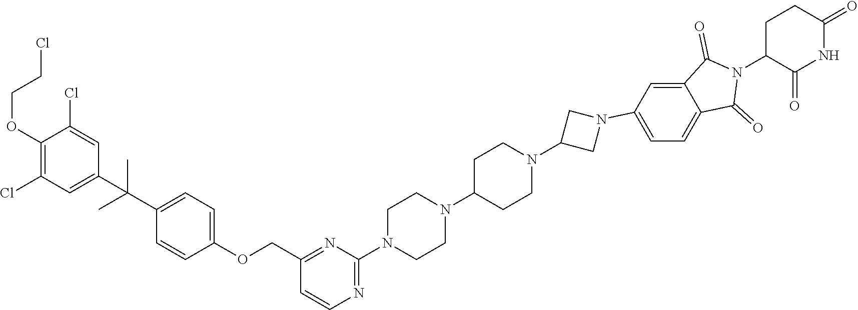 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)c(OCCCl)c(Cl)c1]

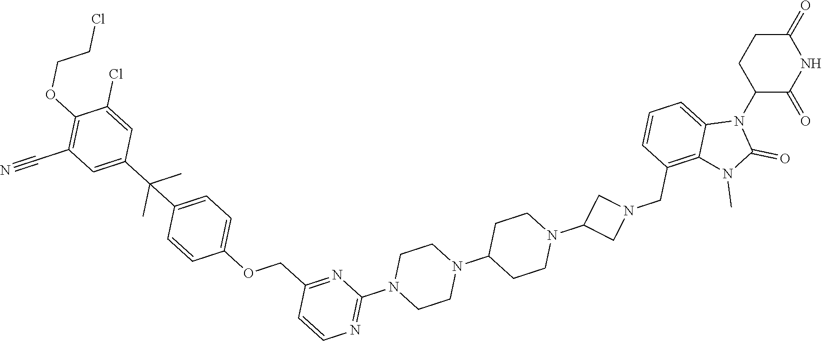 [SMILES: Cn1c(=O)n(C2CCC(=O)NC2=O)c2cccc(CN3CC(N4CCC(N5CCN(c6nccc(COc7ccc(C(C)(C)c8cc(Cl)c(OCCCl)c(C#N)c8)cc7)n6)CC5)CC4)C3)c21]

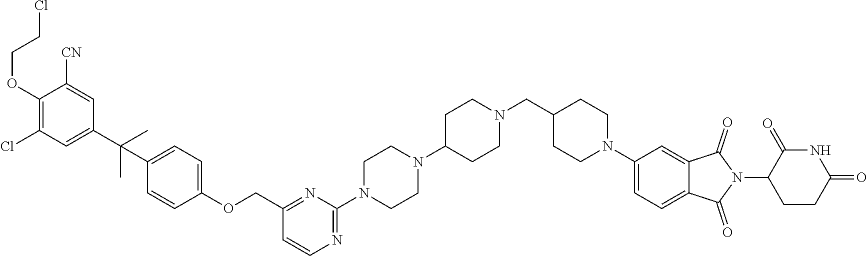 [SMILES: CC(C)(c1ccc(OCc2ccnc(N3CCN(C4CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

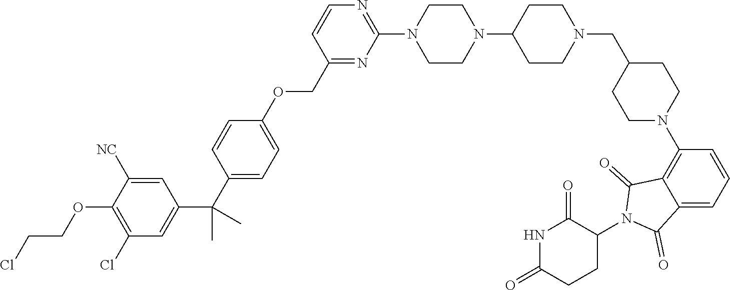 [SMILES: CC(C)(c1ccc(OCc2ccnc(N3CCN(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

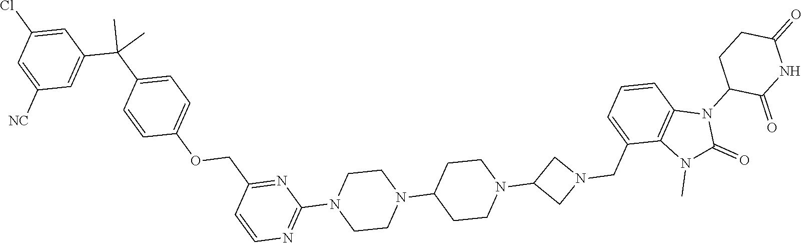 [SMILES: Cn1c(=O)n(C2CCC(=O)NC2=O)c2cccc(CN3CC(N4CCC(N5CCN(c6nccc(COc7ccc(C(C)(C)c8cc(Cl)cc(C#N)c8)cc7)n6)CC5)CC4)C3)c21]

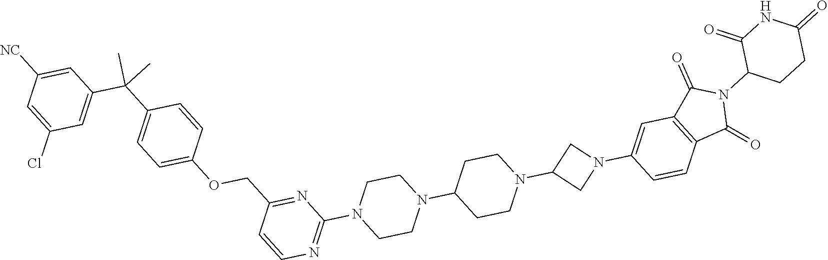 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)cc(C#N)c1]

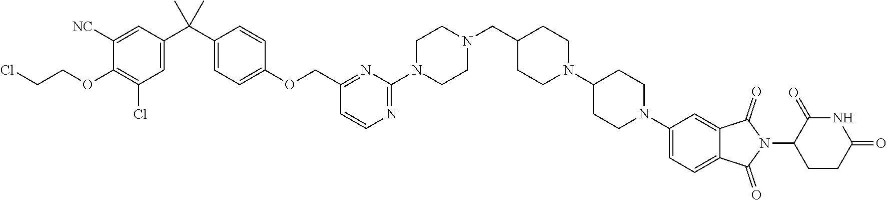 [SMILES: CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)c(OCCCl)c(C#N)c1]

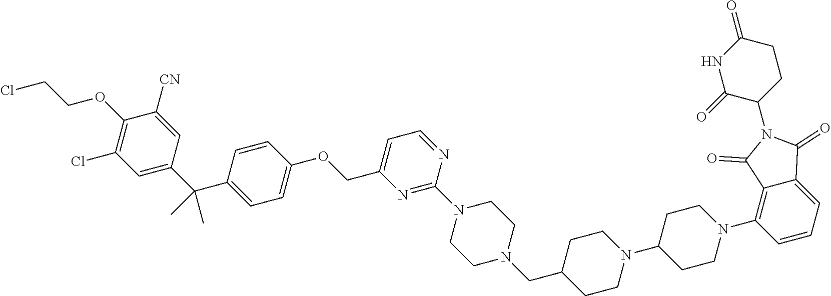 [SMILES: CC(C)(c1ccc(OCc2ccnc(N3CCN(CC4CCN(C5CCN(c6cccc7c6C(=O)N(C6CCC(=O)NC6=O)C7=O)CC5)CC4)CC3)n2)cc1)c1cc(Cl)c(OCCCl)c(C#N)c1]

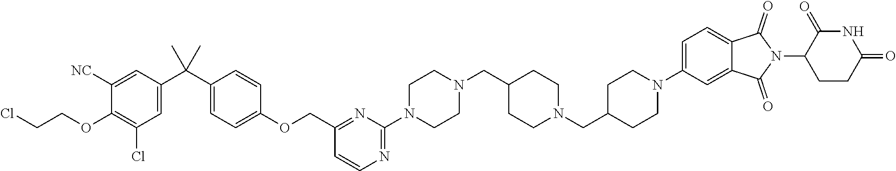 [SMILES: CC(C)(c1ccc(OCc2ccnc(N3CCN(CC4CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

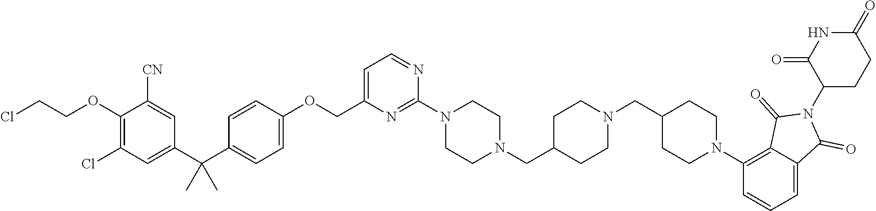 [SMILES: CC(C)(c1ccc(OCc2ccnc(N3CCN(CC4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

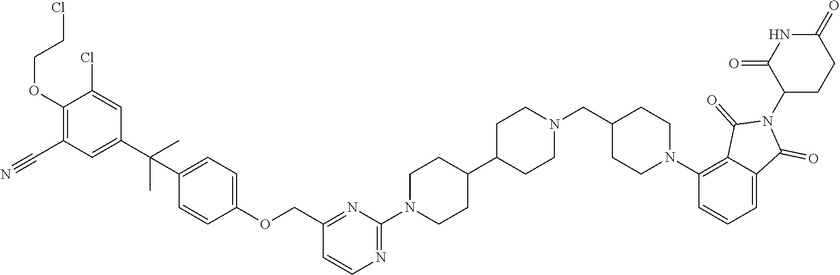 [SMILES: CC(C)(c1ccc(OCc2ccnc(N3CCC(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

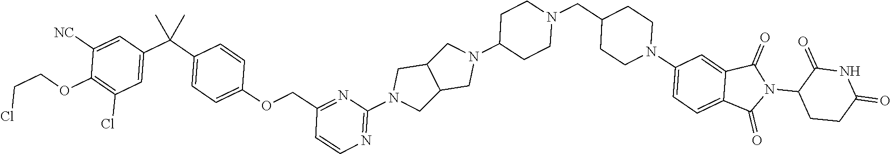 [SMILES: CC(C)(c1ccc(OCc2ccnc(N3CC4CN(C5CCN(CC6CCN(c7ccc8c(c7)C(=O)N(C7CCC(=O)NC7=O)C8=O)CC6)CC5)CC4C3)n2)cc1)c1cc(Cl)c(OCCCl)c(C#N)c1]

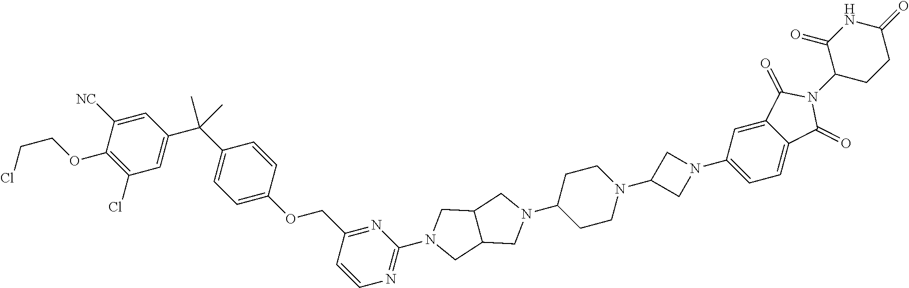 [SMILES: CC(C)(c1ccc(OCc2ccnc(N3CC4CN(C5CCN(C6CN(c7ccc8c(c7)C(=O)N(C7CCC(=O)NC7=O)C8=O)C6)CC5)CC4C3)n2)cc1)c1cc(Cl)c(OCCCl)c(C#N)c1]

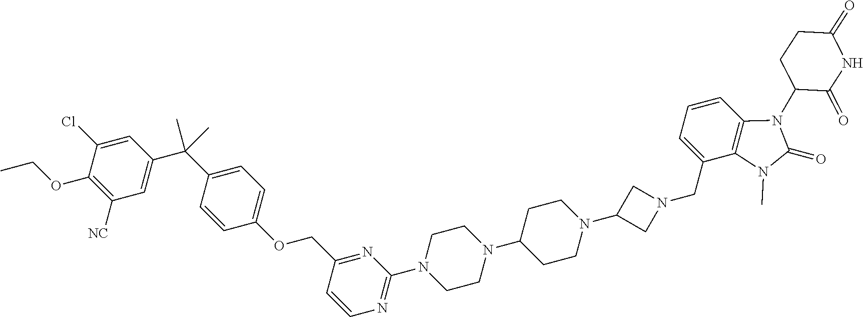 [SMILES: CCOc1c(Cl)cc(C(C)(C)c2ccc(OCc3ccnc(N4CCN(C5CCN(C6CN(Cc7cccc8c7n(C)c(=O)n8C7CCC(=O)NC7=O)C6)CC5)CC4)n3)cc2)cc1C#N]

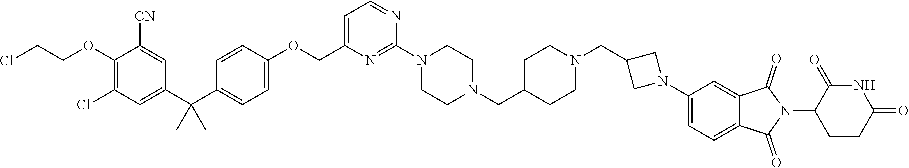 [SMILES: CC(C)(c1ccc(OCc2ccnc(N3CCN(CC4CCN(CC5CN(c6ccc7c(c6)C(=O)N(C6CCC(=O)NC6=O)C7=O)C5)CC4)CC3)n2)cc1)c1cc(Cl)c(OCCCl)c(C#N)c1]

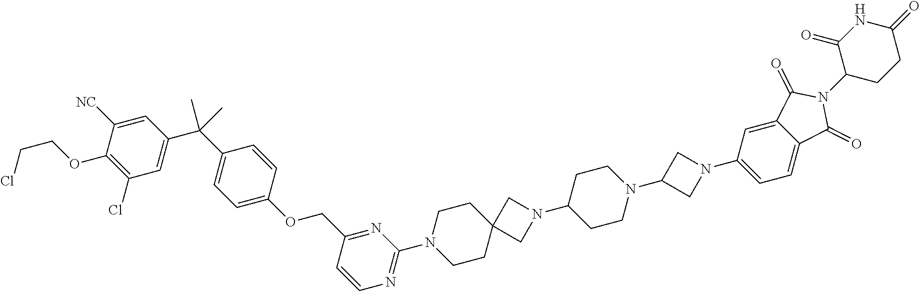 [SMILES: CC(C)(c1ccc(OCc2ccnc(N3CCC4(CC3)CN(C3CCN(C5CN(c6ccc7c(c6)C(=O)N(C6CCC(=O)NC6=O)C7=O)C5)CC3)C4)n2)cc1)c1cc(Cl)c(OCCCl)c(C#N)c1]

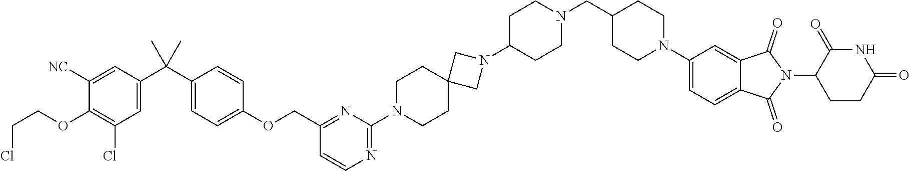 [SMILES: CC(C)(c1ccc(OCc2ccnc(N3CCC4(CC3)CN(C3CCN(CC5CCN(c6ccc7c(c6)C(=O)N(C6CCC(=O)NC6=O)C7=O)CC5)CC3)C4)n2)cc1)c1cc(Cl)c(OCCCl)c(C#N)c1]

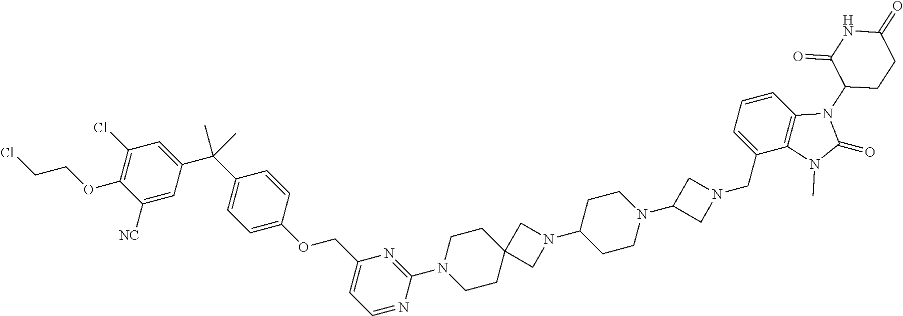 [SMILES: Cn1c(=O)n(C2CCC(=O)NC2=O)c2cccc(CN3CC(N4CCC(N5CC6(CCN(c7nccc(COc8ccc(C(C)(C)c9cc(Cl)c(OCCCl)c(C#N)c9)cc8)n7)CC6)C5)CC4)C3)c21]

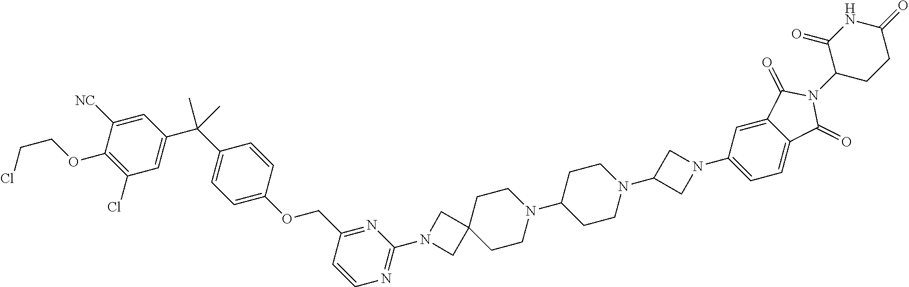 [SMILES: CC(C)(c1ccc(OCc2ccnc(N3CC4(CCN(C5CCN(C6CN(c7ccc8c(c7)C(=O)N(C7CCC(=O)NC7=O)C8=O)C6)CC5)CC4)C3)n2)cc1)c1cc(Cl)c(OCCCl)c(C#N)c1]

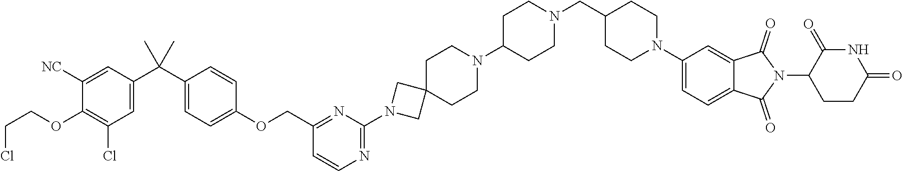 [SMILES: CC(C)(c1ccc(OCc2ccnc(N3CC4(CCN(C5CCN(CC6CCN(c7ccc8c(c7)C(=O)N(C7CCC(=O)NC7=O)C8=O)CC6)CC5)CC4)C3)n2)cc1)c1cc(Cl)c(OCCCl)c(C#N)c1]

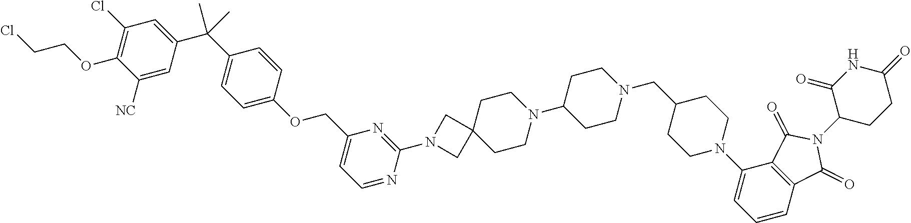 [SMILES: CC(C)(c1ccc(OCc2ccnc(N3CC4(CCN(C5CCN(CC6CCN(c7cccc8c7C(=O)N(C7CCC(=O)NC7=O)C8=O)CC6)CC5)CC4)C3)n2)cc1)c1cc(Cl)c(OCCCl)c(C#N)c1]

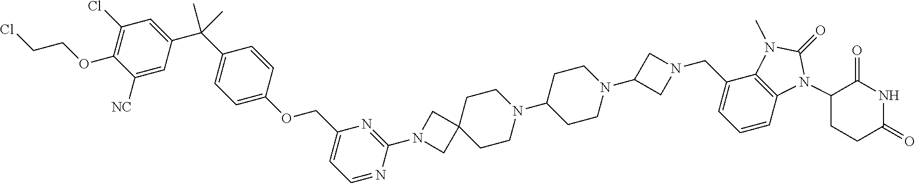 [SMILES: Cn1c(=O)n(C2CCC(=O)NC2=O)c2cccc(CN3CC(N4CCC(N5CCC6(CC5)CN(c5nccc(COc7ccc(C(C)(C)c8cc(Cl)c(OCCCl)c(C#N)c8)cc7)n5)C6)CC4)C3)c21]

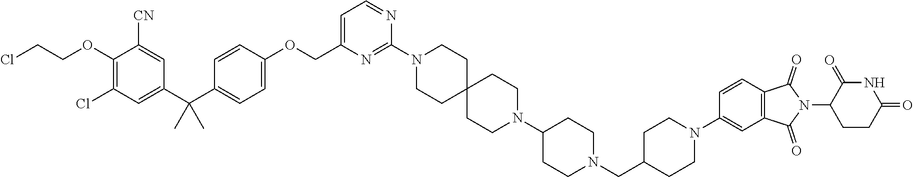 [SMILES: CC(C)(c1ccc(OCc2ccnc(N3CCC4(CC3)CCN(C3CCN(CC5CCN(c6ccc7c(c6)C(=O)N(C6CCC(=O)NC6=O)C7=O)CC5)CC3)CC4)n2)cc1)c1cc(Cl)c(OCCCl)c(C#N)c1]

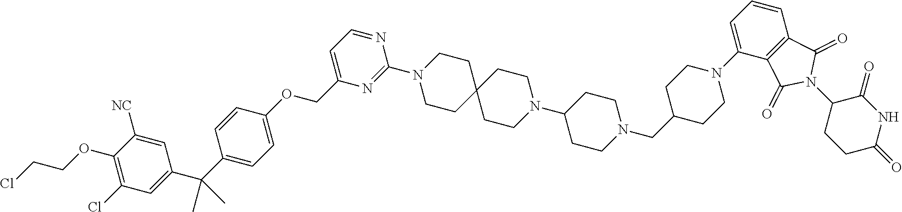 [SMILES: CC(C)(c1ccc(OCc2ccnc(N3CCC4(CC3)CCN(C3CCN(CC5CCN(c6cccc7c6C(=O)N(C6CCC(=O)NC6=O)C7=O)CC5)CC3)CC4)n2)cc1)c1cc(Cl)c(OCCCl)c(C#N)c1]

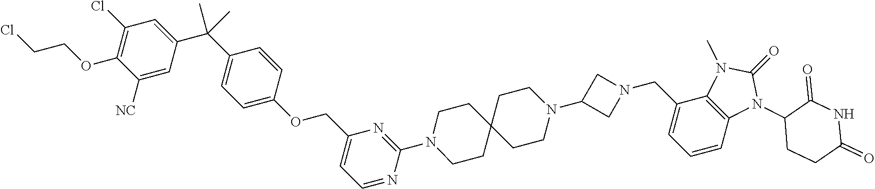 [SMILES: Cn1c(=O)n(C2CCC(=O)NC2=O)c2cccc(CN3CC(N4CCC5(CCN(c6nccc(COc7ccc(C(C)(C)c8cc(Cl)c(OCCCl)c(C#N)c8)cc7)n6)CC5)CC4)C3)c21]

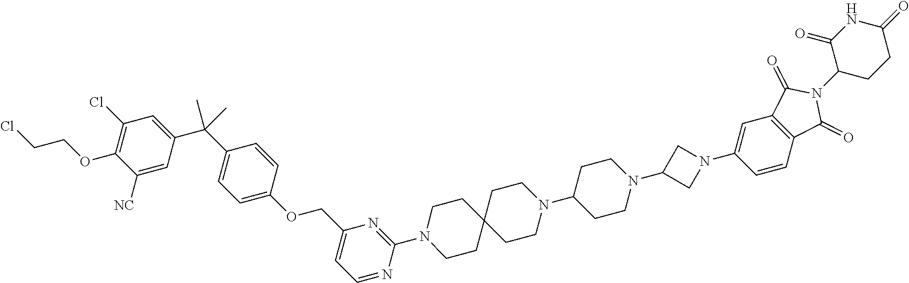 [SMILES: CC(C)(c1ccc(OCc2ccnc(N3CCC4(CC3)CCN(C3CCN(C5CN(c6ccc7c(c6)C(=O)N(C6CCC(=O)NC6=O)C7=O)C5)CC3)CC4)n2)cc1)c1cc(Cl)c(OCCCl)c(C#N)c1]

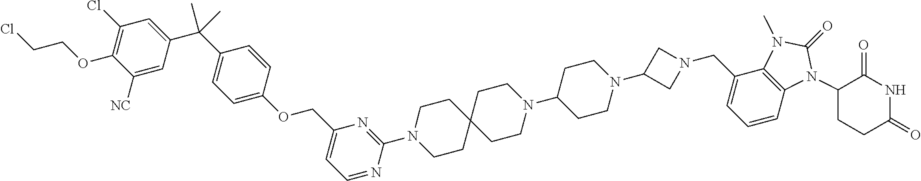 [SMILES: Cn1c(=O)n(C2CCC(=O)NC2=O)c2cccc(CN3CC(N4CCC(N5CCC6(CCN(c7nccc(COc8ccc(C(C)(C)c9cc(Cl)c(OCCCl)c(C#N)c9)cc8)n7)CC6)CC5)CC4)C3)c21]

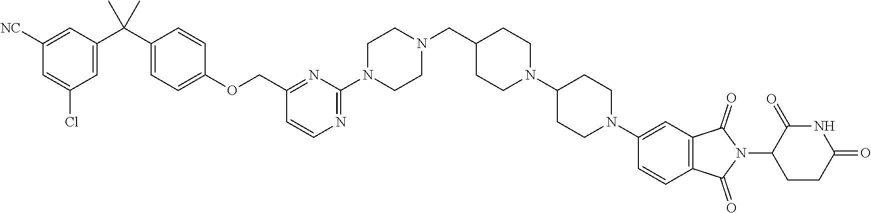 [SMILES: CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)cc(C#N)c1]

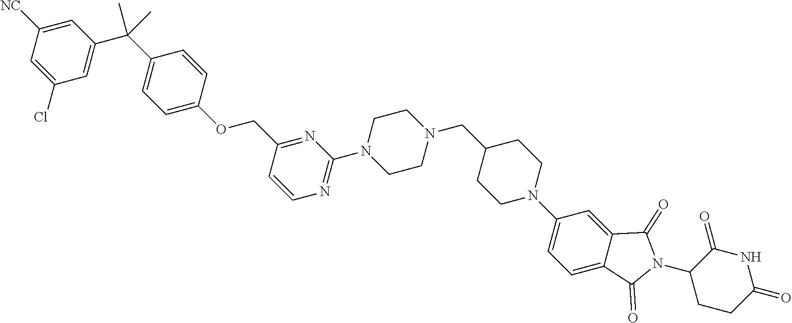 [SMILES: CC(C)(c1ccc(OCc2ccnc(N3CCN(CC4CCN(c5ccc6c(c5)C(=O)N(C5CCC(=O)NC5=O)C6=O)CC4)CC3)n2)cc1)c1cc(Cl)cc(C#N)c1]

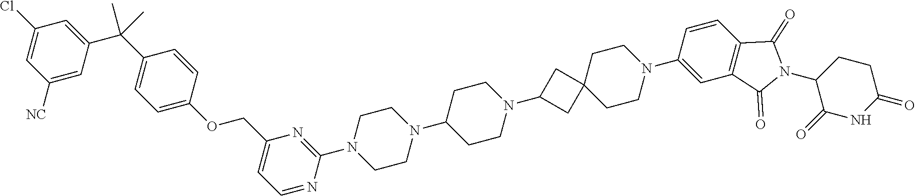 [SMILES: CC(C)(c1ccc(OCc2ccnc(N3CCN(C4CCN(C5CC6(CCN(c7ccc8c(c7)C(=O)N(C7CCC(=O)NC7=O)C8=O)CC6)C5)CC4)CC3)n2)cc1)c1cc(Cl)cc(C#N)c1]

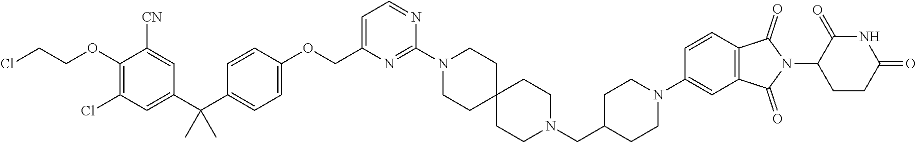 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

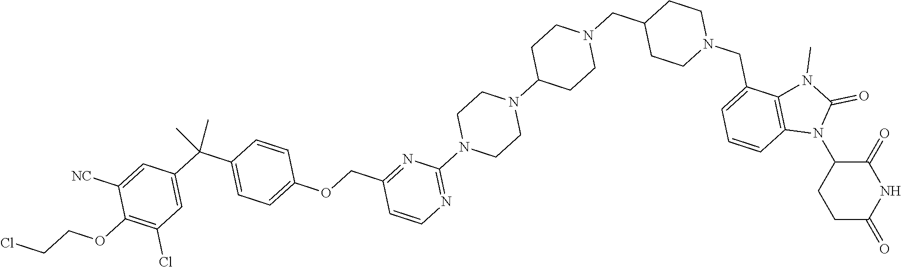 [SMILES: Cn1c(=O)n(C2CCC(=O)NC2=O)c2cccc(CN3CCC(CN4CCC(N5CCN(c6nccc(COc7ccc(C(C)(C)c8cc(Cl)c(OCCCl)c(C#N)c8)cc7)n6)CC5)CC4)CC3)c21]

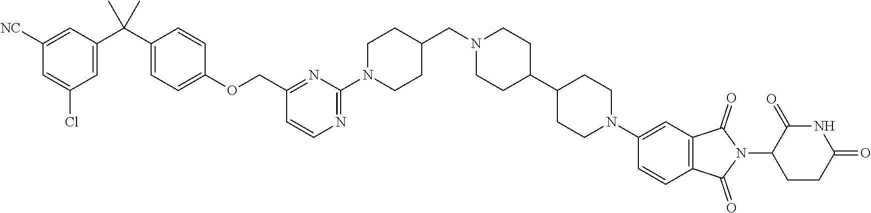 [SMILES: CC(C)(c1ccc(OCc2ccnc(N3CCC(CN4CCC(C5CCN(c6ccc7c(c6)C(=O)N(C6CCC(=O)NC6=O)C7=O)CC5)CC4)CC3)n2)cc1)c1cc(Cl)cc(C#N)c1]

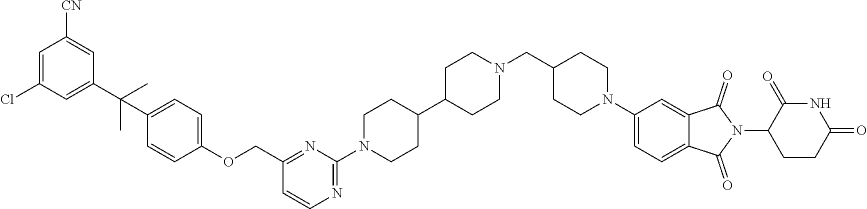 [SMILES: CC(C)(c1ccc(OCc2ccnc(N3CCC(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

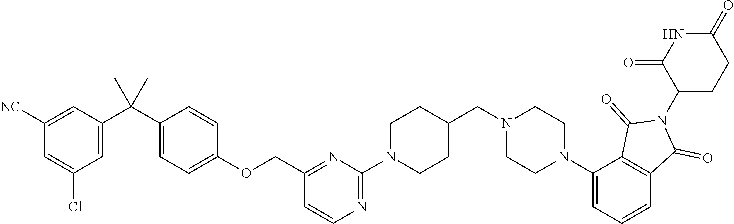 [SMILES: CC(C)(c1ccc(OCc2ccnc(N3CCC(CN4CCN(c5cccc6c5C(=O)N(C5CCC(=O)NC5=O)C6=O)CC4)CC3)n2)cc1)c1cc(Cl)cc(C#N)c1]

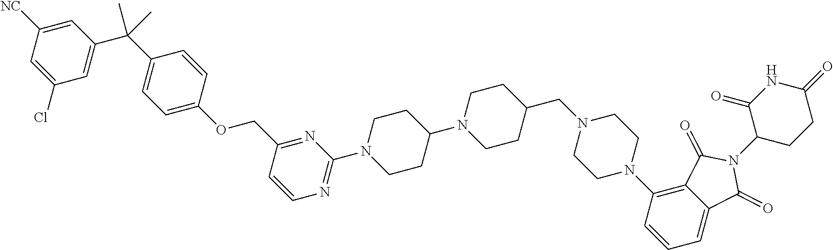 [SMILES: CC(C)(c1ccc(OCc2ccnc(N3CCC(N4CCC(CN5CCN(c6cccc7c6C(=O)N(C6CCC(=O)NC6=O)C7=O)CC5)CC4)CC3)n2)cc1)c1cc(Cl)cc(C#N)c1]

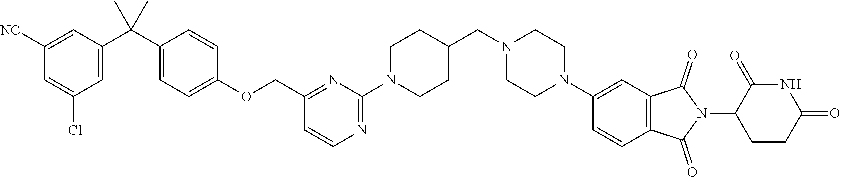 [SMILES: CC(C)(c1ccc(OCc2ccnc(N3CCC(CN4CCN(c5ccc6c(c5)C(=O)N(C5CCC(=O)NC5=O)C6=O)CC4)CC3)n2)cc1)c1cc(Cl)cc(C#N)c1]

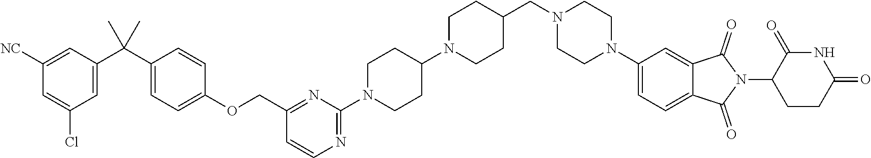 [SMILES: CC(C)(c1ccc(OCc2ccnc(N3CCC(N4CCC(CN5CCN(c6ccc7c(c6)C(=O)N(C6CCC(=O)NC6=O)C7=O)CC5)CC4)CC3)n2)cc1)c1cc(Cl)cc(C#N)c1]

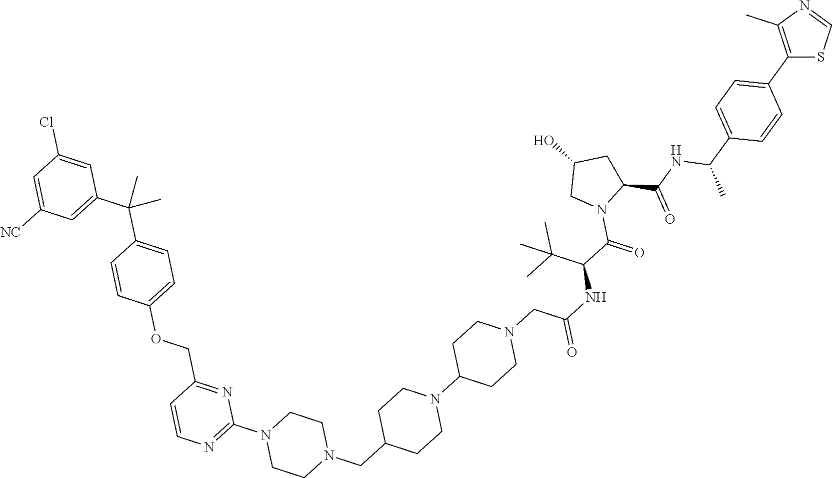 [SMILES: Cc1ncsc1-c1ccc([C@H](C)NC(=O)[C@@H]2C[C@@H](O)CN2C(=O)[C@@H](NC(=O)CN2CCC(N3CCC(CN4CCN(c5nccc(COc6ccc(C(C)(C)c7cc(Cl)cc(C#N)c7)cc6)n5)CC4)CC3)CC2)C(C)(C)C)cc1]

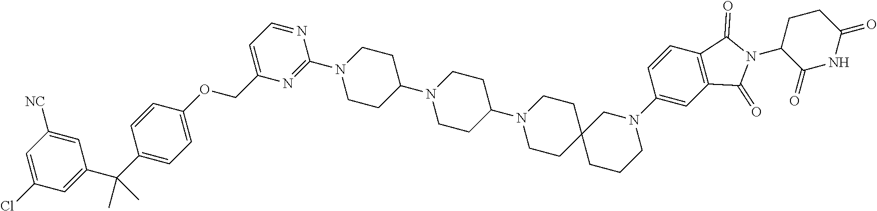 [SMILES: CC(C)(c1ccc(OCc2ccnc(N3CCC(N4CCC(N5CCC6(CCCN(c7ccc8c(c7)C(=O)N(C7CCC(=O)NC7=O)C8=O)C6)CC5)CC4)CC3)n2)cc1)c1cc(Cl)cc(C#N)c1]

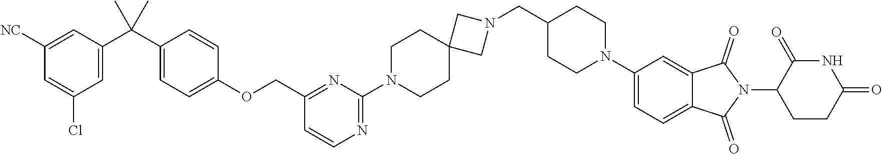 [SMILES: CC(C)(c1ccc(OCc2ccnc(N3CCC4(CC3)CN(CC3CCN(c5ccc6c(c5)C(=O)N(C5CCC(=O)NC5=O)C6=O)CC3)C4)n2)cc1)c1cc(Cl)cc(C#N)c1]

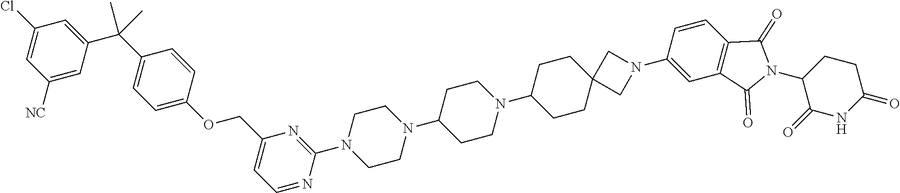 [SMILES: CC(C)(c1ccc(OCc2ccnc(N3CCN(C4CCN(C5CCC6(CC5)CN(c5ccc7c(c5)C(=O)N(C5CCC(=O)NC5=O)C7=O)C6)CC4)CC3)n2)cc1)c1cc(Cl)cc(C#N)c1]

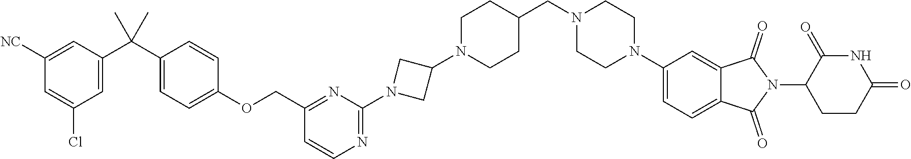 [SMILES: CC(C)(c1ccc(OCc2ccnc(N3CC(N4CCC(CN5CCN(c6ccc7c(c6)C(=O)N(C6CCC(=O)NC6=O)C7=O)CC5)CC4)C3)n2)cc1)c1cc(Cl)cc(C#N)c1]

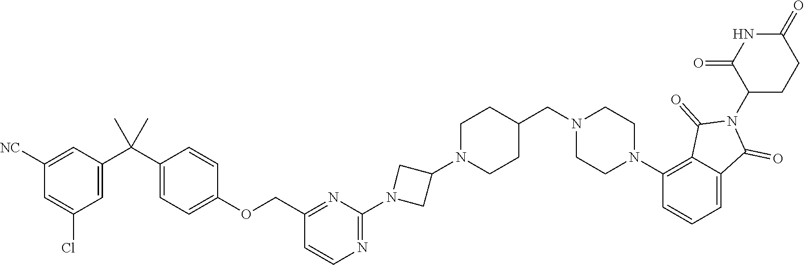 [SMILES: CC(C)(c1ccc(OCc2ccnc(N3CC(N4CCC(CN5CCN(c6cccc7c6C(=O)N(C6CCC(=O)NC6=O)C7=O)CC5)CC4)C3)n2)cc1)c1cc(Cl)cc(C#N)c1]

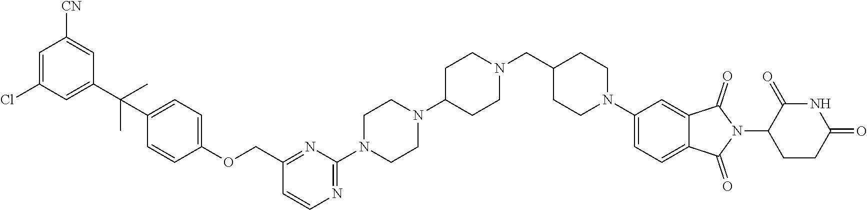 [SMILES: CC(C)(c1ccc(OCc2ccnc(N3CCN(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

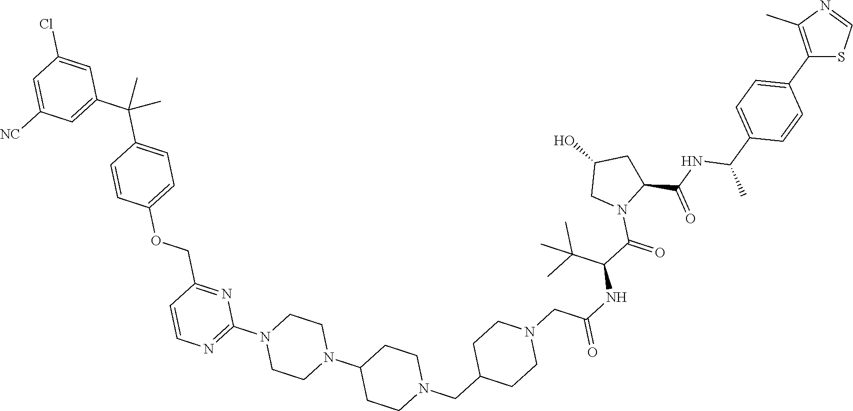 [SMILES: Cc1ncsc1-c1ccc([C@H](C)NC(=O)[C@@H]2C[C@@H](O)CN2C(=O)[C@@H](NC(=O)CN2CCC(CN3CCC(N4CCN(c5nccc(COc6ccc(C(C)(C)c7cc(Cl)cc(C#N)c7)cc6)n5)CC4)CC3)CC2)C(C)(C)C)cc1]

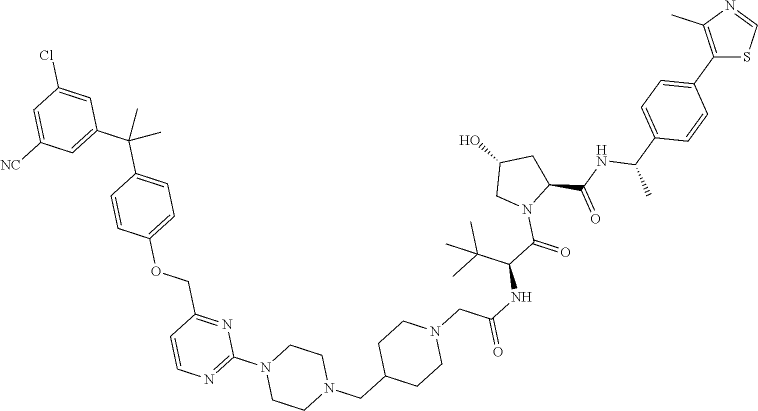 [SMILES: Cc1ncsc1-c1ccc([C@H](C)NC(=O)[C@@H]2C[C@@H](O)CN2C(=O)[C@@H](NC(=O)CN2CCC(CN3CCN(c4nccc(COc5ccc(C(C)(C)c6cc(Cl)cc(C#N)c6)cc5)n4)CC3)CC2)C(C)(C)C)cc1]

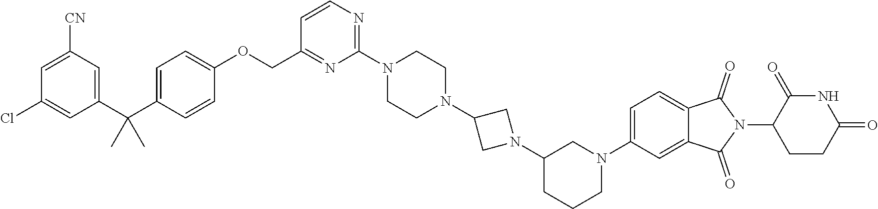 [SMILES: CC(C)(c1ccc(OCc2ccnc(N3CCN(C4CN(C5CCCN(c6ccc7c(c6)C(=O)N(C6CCC(=O)NC6=O)C7=O)C5)C4)CC3)n2)cc1)c1cc(Cl)cc(C#N)c1]

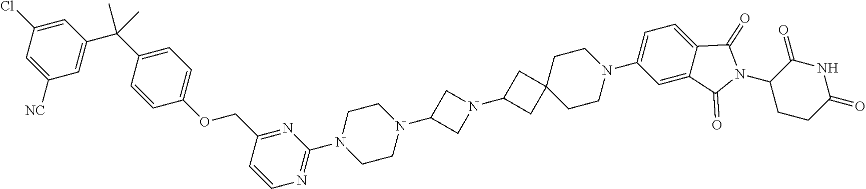 [SMILES: CC(C)(c1ccc(OCc2ccnc(N3CCN(C4CN(C5CC6(CCN(c7ccc8c(c7)C(=O)N(C7CCC(=O)NC7=O)C8=O)CC6)C5)C4)CC3)n2)cc1)c1cc(Cl)cc(C#N)c1]

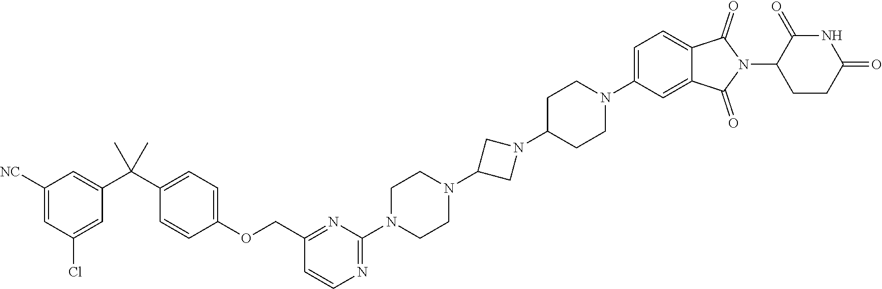 [SMILES: CC(C)(c1ccc(OCc2ccnc(N3CCN(C4CN(C5CCN(c6ccc7c(c6)C(=O)N(C6CCC(=O)NC6=O)C7=O)CC5)C4)CC3)n2)cc1)c1cc(Cl)cc(C#N)c1]

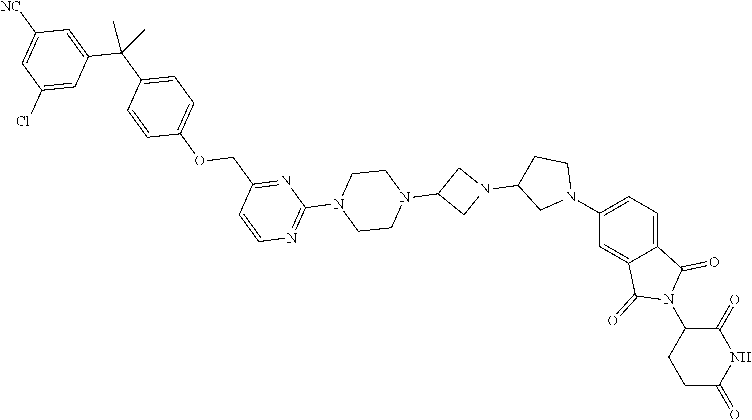 [SMILES: CC(C)(c1ccc(OCc2ccnc(N3CCN(C4CN(C5CCN(c6ccc7c(c6)C(=O)N(C6CCC(=O)NC6=O)C7=O)C5)C4)CC3)n2)cc1)c1cc(Cl)cc(C#N)c1]

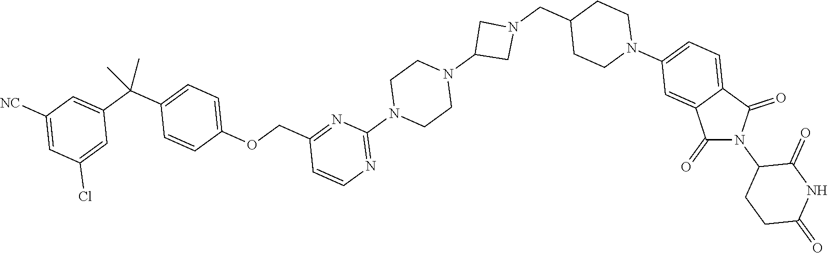 [SMILES: CC(C)(c1ccc(OCc2ccnc(N3CCN(C4CN(CC5CCN(c6ccc7c(c6)C(=O)N(C6CCC(=O)NC6=O)C7=O)CC5)C4)CC3)n2)cc1)c1cc(Cl)cc(C#N)c1]

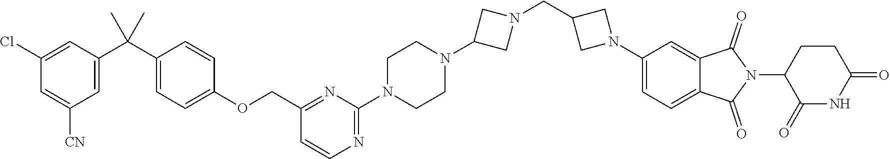 [SMILES: CC(C)(c1ccc(OCc2ccnc(N3CCN(C4CN(CC5CN(c6ccc7c(c6)C(=O)N(C6CCC(=O)NC6=O)C7=O)C5)C4)CC3)n2)cc1)c1cc(Cl)cc(C#N)c1]

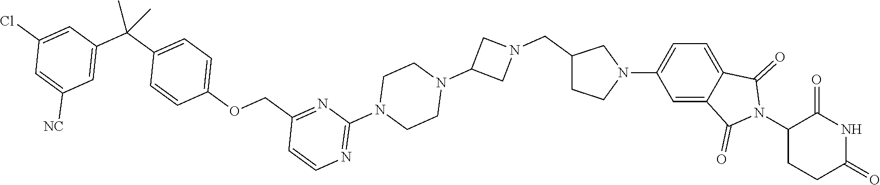 [SMILES: CC(C)(c1ccc(OCc2ccnc(N3CCN(C4CN(CC5CCN(c6ccc7c(c6)C(=O)N(C6CCC(=O)NC6=O)C7=O)C5)C4)CC3)n2)cc1)c1cc(Cl)cc(C#N)c1]

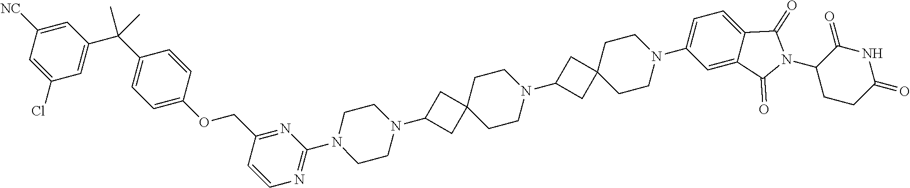 [SMILES: CC(C)(c1ccc(OCc2ccnc(N3CCN(C4CC5(CCN(C6CC7(CCN(c8ccc9c(c8)C(=O)N(C8CCC(=O)NC8=O)C9=O)CC7)C6)CC5)C4)CC3)n2)cc1)c1cc(Cl)cc(C#N)c1]

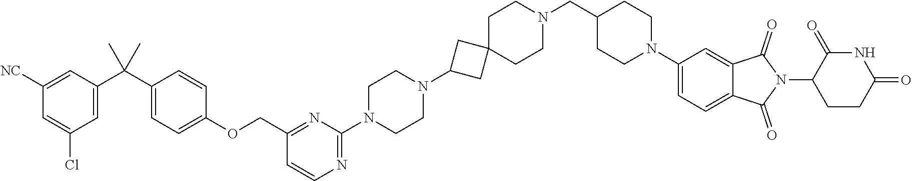 [SMILES: CC(C)(c1ccc(OCc2ccnc(N3CCN(C4CC5(CCN(CC6CCN(c7ccc8c(c7)C(=O)N(C7CCC(=O)NC7=O)C8=O)CC6)CC5)C4)CC3)n2)cc1)c1cc(Cl)cc(C#N)c1]

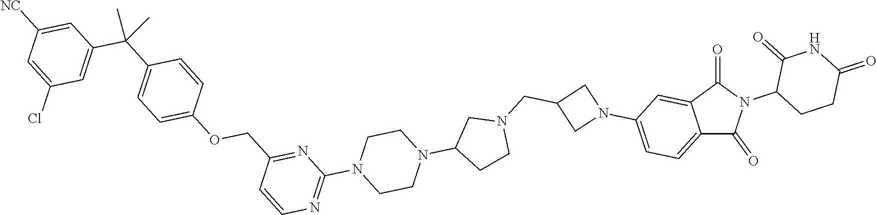 [SMILES: CC(C)(c1ccc(OCc2ccnc(N3CCN(C4CCN(CC5CN(c6ccc7c(c6)C(=O)N(C6CCC(=O)NC6=O)C7=O)C5)C4)CC3)n2)cc1)c1cc(Cl)cc(C#N)c1]

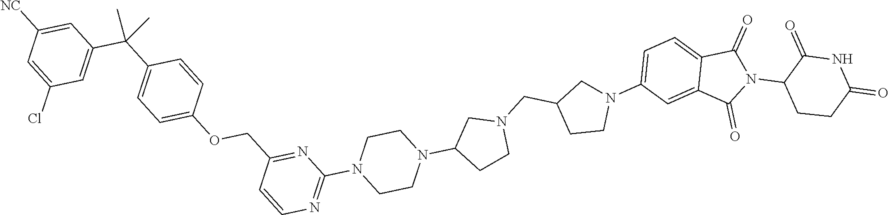 [SMILES: CC(C)(c1ccc(OCc2ccnc(N3CCN(C4CCN(CC5CCN(c6ccc7c(c6)C(=O)N(C6CCC(=O)NC6=O)C7=O)C5)C4)CC3)n2)cc1)c1cc(Cl)cc(C#N)c1]

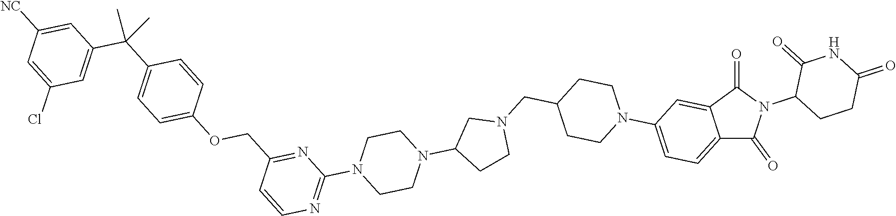 [SMILES: CC(C)(c1ccc(OCc2ccnc(N3CCN(C4CCN(CC5CCN(c6ccc7c(c6)C(=O)N(C6CCC(=O)NC6=O)C7=O)CC5)C4)CC3)n2)cc1)c1cc(Cl)cc(C#N)c1]

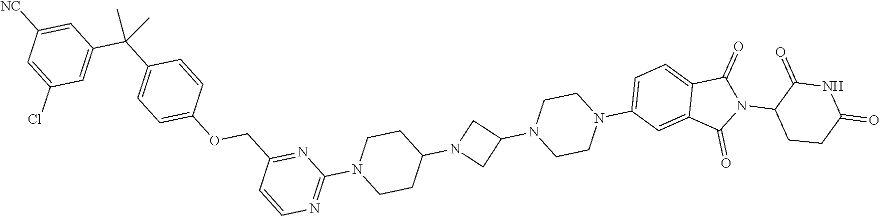 [SMILES: CC(C)(c1ccc(OCc2ccnc(N3CCC(N4CC(N5CCN(c6ccc7c(c6)C(=O)N(C6CCC(=O)NC6=O)C7=O)CC5)C4)CC3)n2)cc1)c1cc(Cl)cc(C#N)c1]

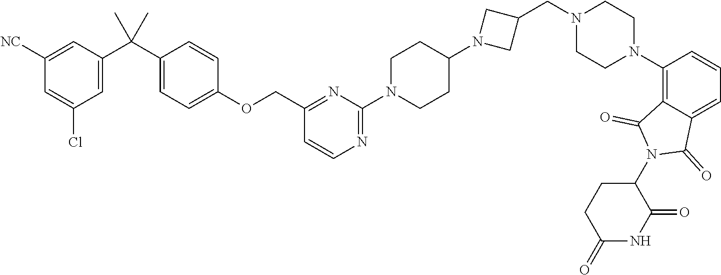 [SMILES: CC(C)(c1ccc(OCc2ccnc(N3CCC(N4CC(CN5CCN(c6cccc7c6C(=O)N(C6CCC(=O)NC6=O)C7=O)CC5)C4)CC3)n2)cc1)c1cc(Cl)cc(C#N)c1]

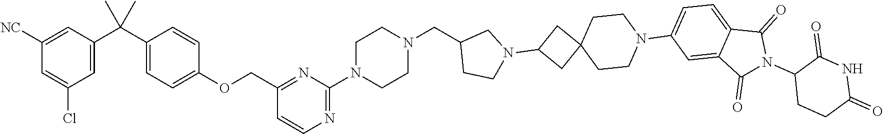 [SMILES: CC(C)(c1ccc(OCc2ccnc(N3CCN(CC4CCN(C5CC6(CCN(c7ccc8c(c7)C(=O)N(C7CCC(=O)NC7=O)C8=O)CC6)C5)C4)CC3)n2)cc1)c1cc(Cl)cc(C#N)c1]

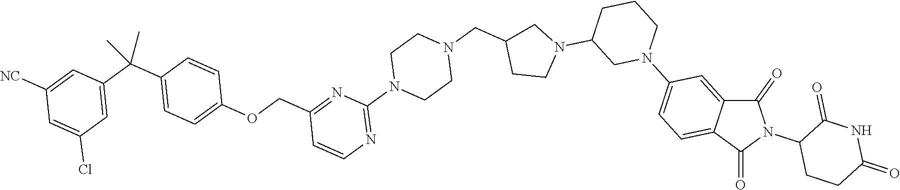 [SMILES: CC(C)(c1ccc(OCc2ccnc(N3CCN(CC4CCN(C5CCCN(c6ccc7c(c6)C(=O)N(C6CCC(=O)NC6=O)C7=O)C5)C4)CC3)n2)cc1)c1cc(Cl)cc(C#N)c1]

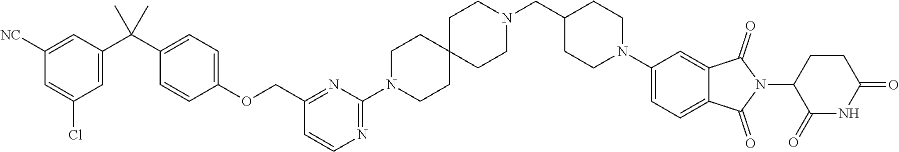 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)cc(C#N)c1]

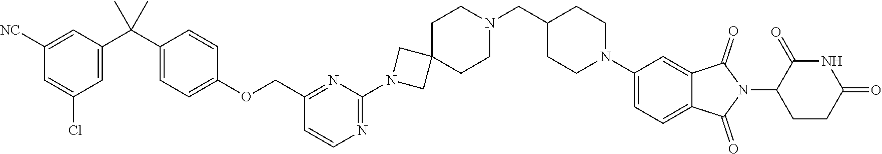 [SMILES: CC(C)(c1ccc(OCc2ccnc(N3CC4(CCN(CC5CCN(c6ccc7c(c6)C(=O)N(C6CCC(=O)NC6=O)C7=O)CC5)CC4)C3)n2)cc1)c1cc(Cl)cc(C#N)c1]

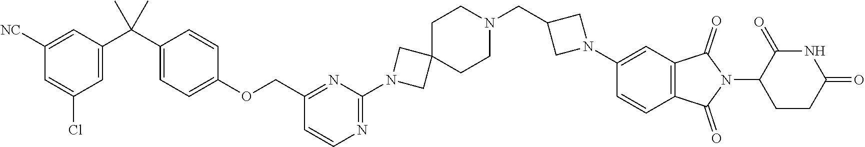 [SMILES: CC(C)(c1ccc(OCc2ccnc(N3CC4(CCN(CC5CN(c6ccc7c(c6)C(=O)N(C6CCC(=O)NC6=O)C7=O)C5)CC4)C3)n2)cc1)c1cc(Cl)cc(C#N)c1]

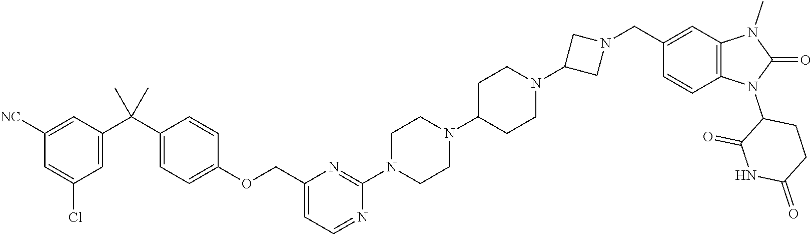 [SMILES: Cn1c(=O)n(C2CCC(=O)NC2=O)c2ccc(CN3CC(N4CCC(N5CCN(c6nccc(COc7ccc(C(C)(C)c8cc(Cl)cc(C#N)c8)cc7)n6)CC5)CC4)C3)cc21]

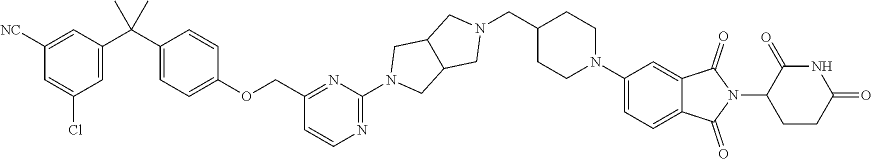 [SMILES: CC(C)(c1ccc(OCc2ccnc(N3CC4CN(CC5CCN(c6ccc7c(c6)C(=O)N(C6CCC(=O)NC6=O)C7=O)CC5)CC4C3)n2)cc1)c1cc(Cl)cc(C#N)c1]

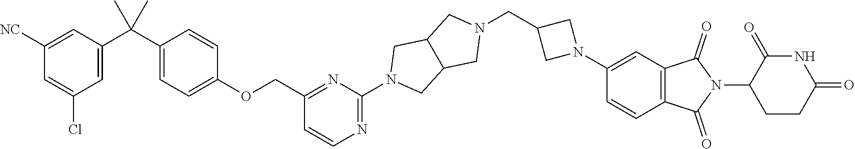 [SMILES: CC(C)(c1ccc(OCc2ccnc(N3CC4CN(CC5CN(c6ccc7c(c6)C(=O)N(C6CCC(=O)NC6=O)C7=O)C5)CC4C3)n2)cc1)c1cc(Cl)cc(C#N)c1]

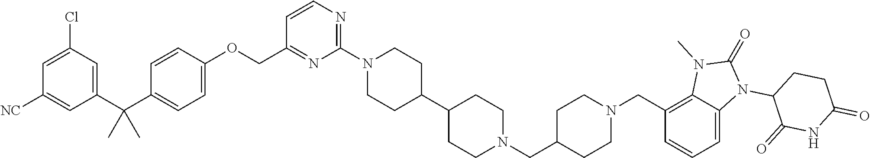 [SMILES: Cn1c(=O)n(C2CCC(=O)NC2=O)c2cccc(CN3CCC(CN4CCC(C5CCN(c6nccc(COc7ccc(C(C)(C)c8cc(Cl)cc(C#N)c8)cc7)n6)CC5)CC4)CC3)c21]

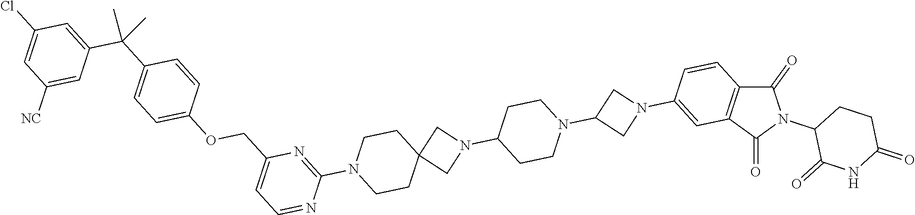 [SMILES: CC(C)(c1ccc(OCc2ccnc(N3CCC4(CC3)CN(C3CCN(C5CN(c6ccc7c(c6)C(=O)N(C6CCC(=O)NC6=O)C7=O)C5)CC3)C4)n2)cc1)c1cc(Cl)cc(C#N)c1]

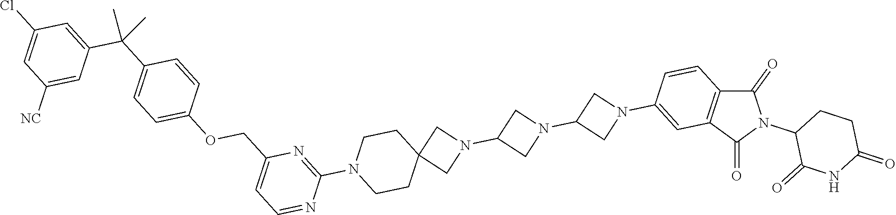 [SMILES: CC(C)(c1ccc(OCc2ccnc(N3CCC4(CC3)CN(C3CN(C5CN(c6ccc7c(c6)C(=O)N(C6CCC(=O)NC6=O)C7=O)C5)C3)C4)n2)cc1)c1cc(Cl)cc(C#N)c1]

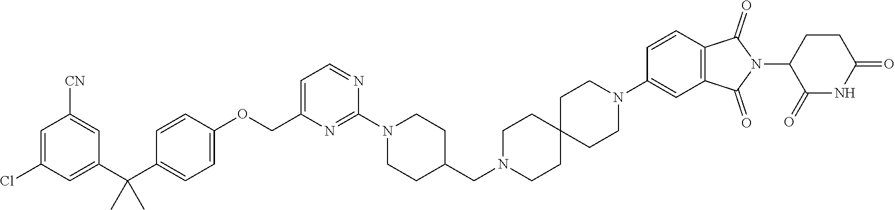 [SMILES: CC(C)(c1ccc(OCc2ccnc(N3CCC(CN4CCC5(CC4)CCN(c4ccc6c(c4)C(=O)N(C4CCC(=O)NC4=O)C6=O)CC5)CC3)n2)cc1)c1cc(Cl)cc(C#N)c1]

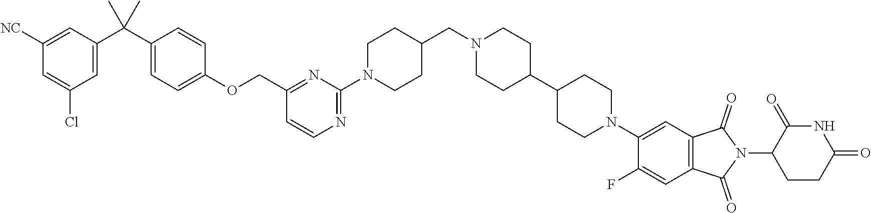 [SMILES: CC(C)(c1ccc(OCc2ccnc(N3CCC(CN4CCC(C5CCN(c6cc7c(cc6F)C(=O)N(C6CCC(=O)NC6=O)C7=O)CC5)CC4)CC3)n2)cc1)c1cc(Cl)cc(C#N)c1]

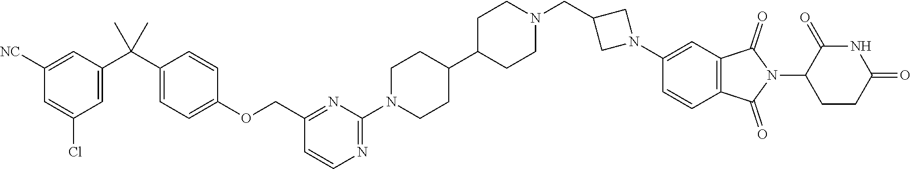 [SMILES: CC(C)(c1ccc(OCc2ccnc(N3CCC(C4CCN(CC5CN(c6ccc7c(c6)C(=O)N(C6CCC(=O)NC6=O)C7=O)C5)CC4)CC3)n2)cc1)c1cc(Cl)cc(C#N)c1]

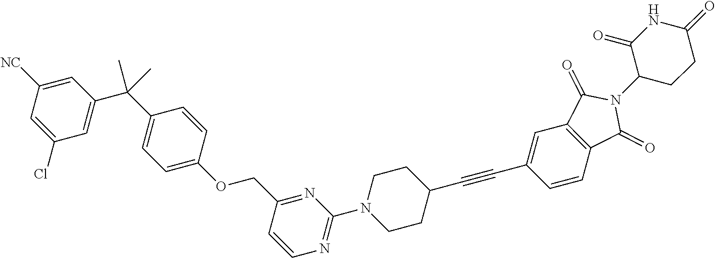 [SMILES: CC(C)(c1ccc(OCc2ccnc(N3CCC(C#Cc4ccc5c(c4)C(=O)N(C4CCC(=O)NC4=O)C5=O)CC3)n2)cc1)c1cc(Cl)cc(C#N)c1]

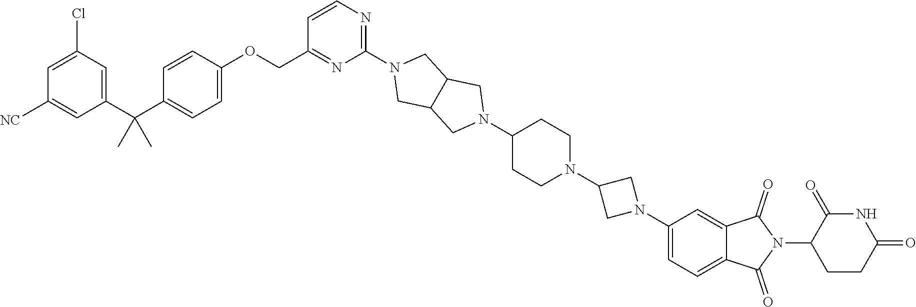 [SMILES: CC(C)(c1ccc(OCc2ccnc(N3CC4CN(C5CCN(C6CN(c7ccc8c(c7)C(=O)N(C7CCC(=O)NC7=O)C8=O)C6)CC5)CC4C3)n2)cc1)c1cc(Cl)cc(C#N)c1]

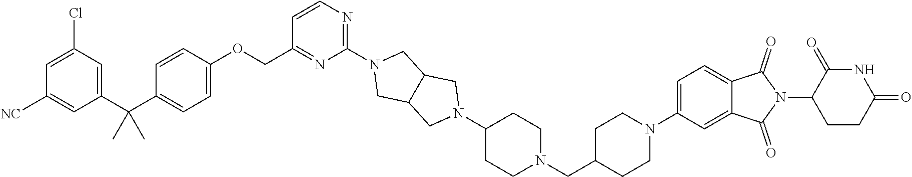 [SMILES: CC(C)(c1ccc(OCc2ccnc(N3CC4CN(C5CCN(CC6CCN(c7ccc8c(c7)C(=O)N(C7CCC(=O)NC7=O)C8=O)CC6)CC5)CC4C3)n2)cc1)c1cc(Cl)cc(C#N)c1]

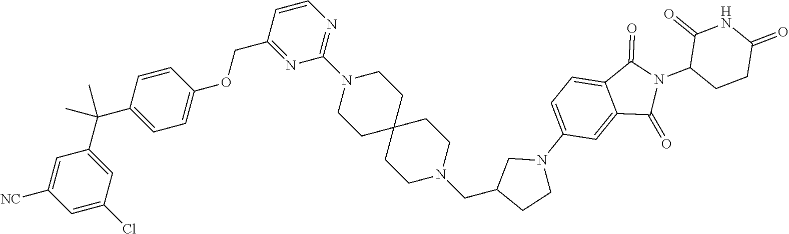 [SMILES: CC(C)(c1ccc(OCc2ccnc(N3CCC4(CCN(CC5CCN(c6ccc7c(c6)C(=O)N(C6CCC(=O)NC6=O)C7=O)C5)CC4)CC3)n2)cc1)c1cc(Cl)cc(C#N)c1]

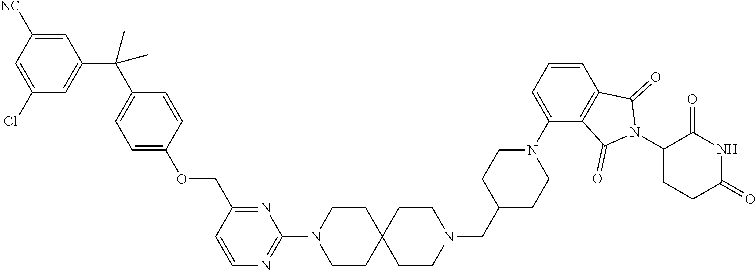 [SMILES: CC(C)(c1ccc(OCc2ccnc(N3CCC4(CCN(CC5CCN(c6cccc7c6C(=O)N(C6CCC(=O)NC6=O)C7=O)CC5)CC4)CC3)n2)cc1)c1cc(Cl)cc(C#N)c1]

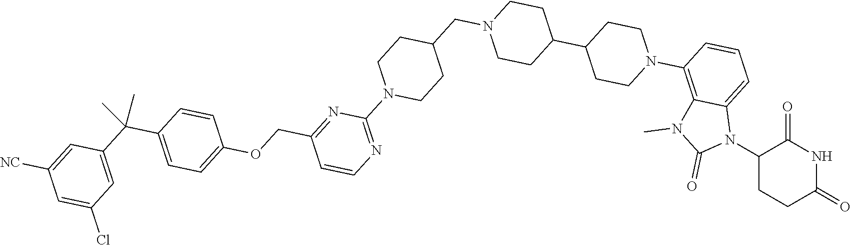 [SMILES: Cn1c(=O)n(C2CCC(=O)NC2=O)c2cccc(N3CCC(C4CCN(CC5CCN(c6nccc(COc7ccc(C(C)(C)c8cc(Cl)cc(C#N)c8)cc7)n6)CC5)CC4)CC3)c21]

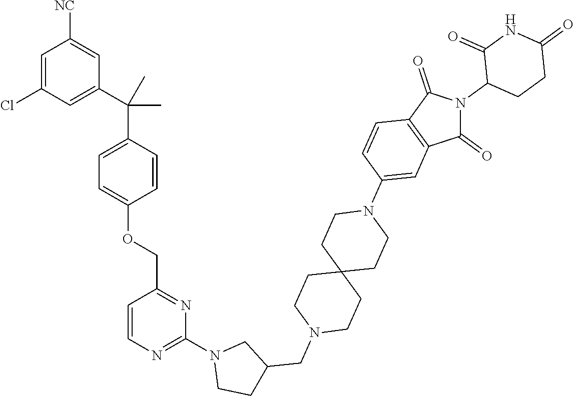 [SMILES: CC(C)(c1ccc(OCc2ccnc(N3CCC(CN4CCC5(CC4)CCN(c4ccc6c(c4)C(=O)N(C4CCC(=O)NC4=O)C6=O)CC5)C3)n2)cc1)c1cc(Cl)cc(C#N)c1]

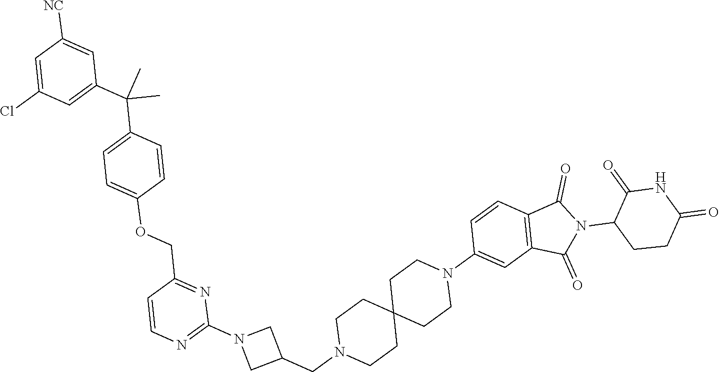 [SMILES: CC(C)(c1ccc(OCc2ccnc(N3CC(CN4CCC5(CC4)CCN(c4ccc6c(c4)C(=O)N(C4CCC(=O)NC4=O)C6=O)CC5)C3)n2)cc1)c1cc(Cl)cc(C#N)c1]

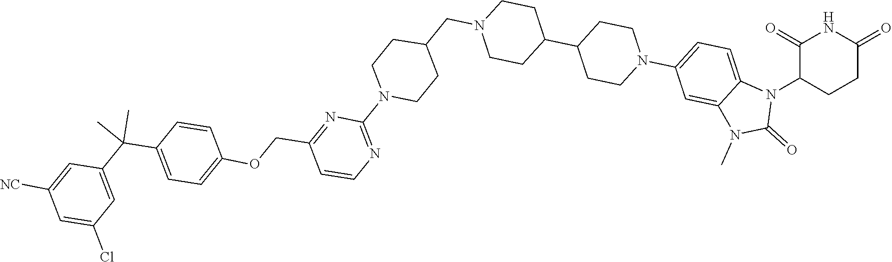 [SMILES: Cn1c(=O)n(C2CCC(=O)NC2=O)c2ccc(N3CCC(C4CCN(CC5CCN(c6nccc(COc7ccc(C(C)(C)c8cc(Cl)cc(C#N)c8)cc7)n6)CC5)CC4)CC3)cc21]

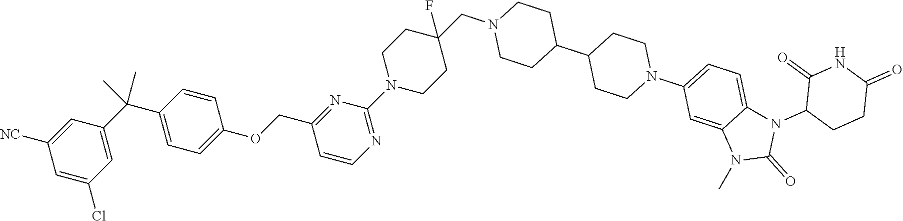 [SMILES: Cn1c(=O)n(C2CCC(=O)NC2=O)c2ccc(N3CCC(C4CCN(CC5(F)CCN(c6nccc(COc7ccc(C(C)(C)c8cc(Cl)cc(C#N)c8)cc7)n6)CC5)CC4)CC3)cc21]

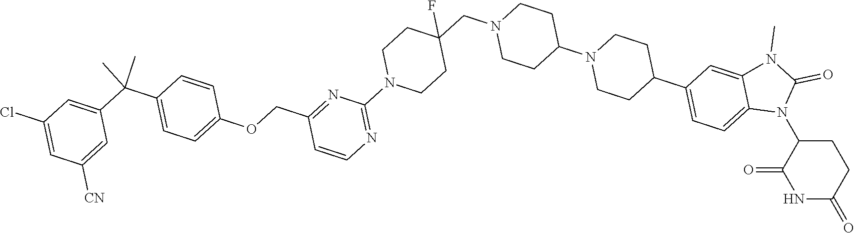 [SMILES: Cn1c(=O)n(C2CCC(=O)NC2=O)c2ccc(C3CCN(C4CCN(CC5(F)CCN(c6nccc(COc7ccc(C(C)(C)c8cc(Cl)cc(C#N)c8)cc7)n6)CC5)CC4)CC3)cc21]

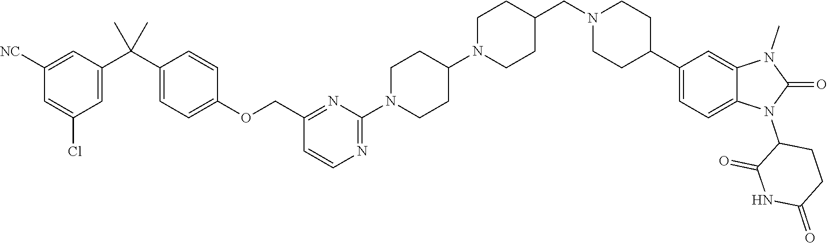 [SMILES: Cn1c(=O)n(C2CCC(=O)NC2=O)c2ccc(C3CCN(CC4CCN(C5CCN(c6nccc(COc7ccc(C(C)(C)c8cc(Cl)cc(C#N)c8)cc7)n6)CC5)CC4)CC3)cc21]

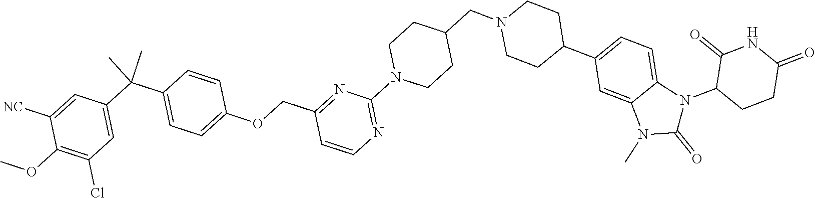 [SMILES: COc1c(Cl)cc(C(C)(C)c2ccc(OCc3ccnc(N4CCC(CN5CCC(c6ccc7c(c6)n(C)c(=O)n7C6CCC(=O)NC6=O)CC5)CC4)n3)cc2)cc1C#N]

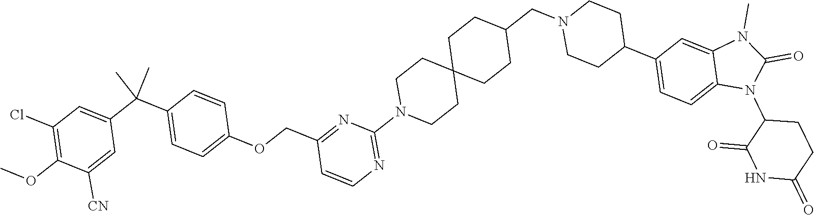 [SMILES: COc1c(Cl)cc(C(C)(C)c2ccc(OCc3ccnc(N4CCC5(CCC(CN6CCC(c7ccc8c(c7)n(C)c(=O)n8C7CCC(=O)NC7=O)CC6)CC5)CC4)n3)cc2)cc1C#N]

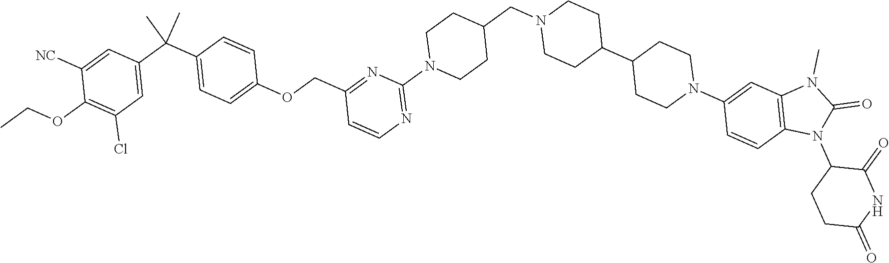 [SMILES: CCOc1c(Cl)cc(C(C)(C)c2ccc(OCc3ccnc(N4CCC(CN5CCC(C6CCN(c7ccc8c(c7)n(C)c(=O)n8C7CCC(=O)NC7=O)CC6)CC5)CC4)n3)cc2)cc1C#N]

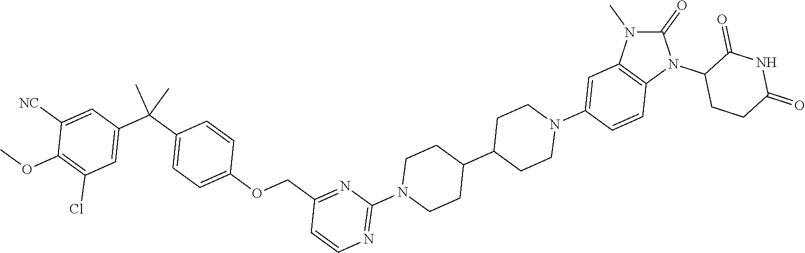 [SMILES: COc1c(Cl)cc(C(C)(C)c2ccc(OCc3ccnc(N4CCC(C5CCN(c6ccc7c(c6)n(C)c(=O)n7C6CCC(=O)NC6=O)CC5)CC4)n3)cc2)cc1C#N]

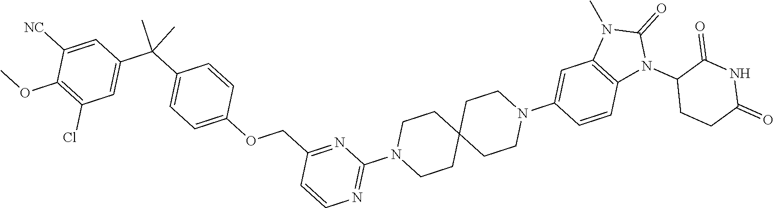 [SMILES: COc1c(Cl)cc(C(C)(C)c2ccc(OCc3ccnc(N4CCC5(CCN(c6ccc7c(c6)n(C)c(=O)n7C6CCC(=O)NC6=O)CC5)CC4)n3)cc2)cc1C#N]

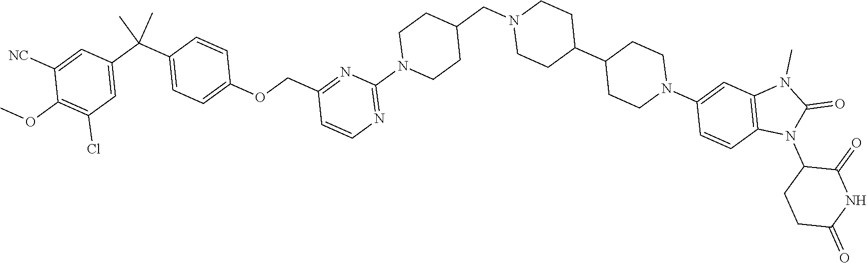 [SMILES: COc1c(Cl)cc(C(C)(C)c2ccc(OCc3ccnc(N4CCC(CN5CCC(C6CCN(c7ccc8c(c7)n(C)c(=O)n8C7CCC(=O)NC7=O)CC6)CC5)CC4)n3)cc2)cc1C#N]

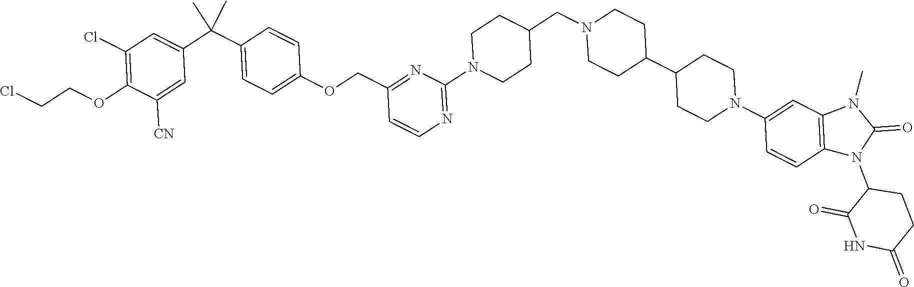 [SMILES: Cn1c(=O)n(C2CCC(=O)NC2=O)c2ccc(N3CCC(C4CCN(CC5CCN(c6nccc(COc7ccc(C(C)(C)c8cc(Cl)c(OCCCl)c(C#N)c8)cc7)n6)CC5)CC4)CC3)cc21]

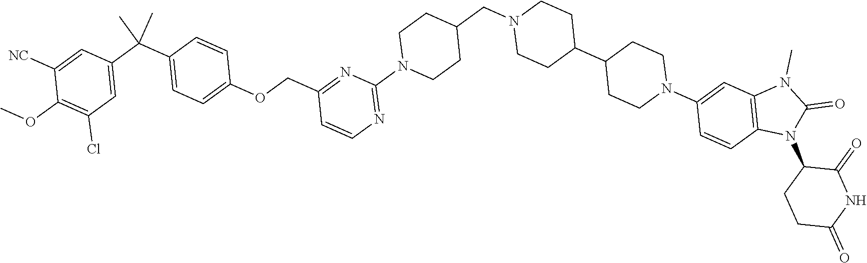 [SMILES: COc1c(Cl)cc(C(C)(C)c2ccc(OCc3ccnc(N4CCC(CN5CCC(C6CCN(c7ccc8c(c7)n(C)c(=O)n8[C@@H]7CCC(=O)NC7=O)CC6)CC5)CC4)n3)cc2)cc1C#N]

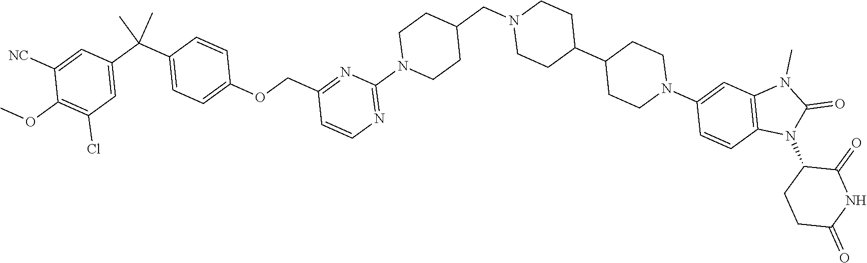 [SMILES: COc1c(Cl)cc(C(C)(C)c2ccc(OCc3ccnc(N4CCC(CN5CCC(C6CCN(c7ccc8c(c7)n(C)c(=O)n8[C@H]7CCC(=O)NC7=O)CC6)CC5)CC4)n3)cc2)cc1C#N]

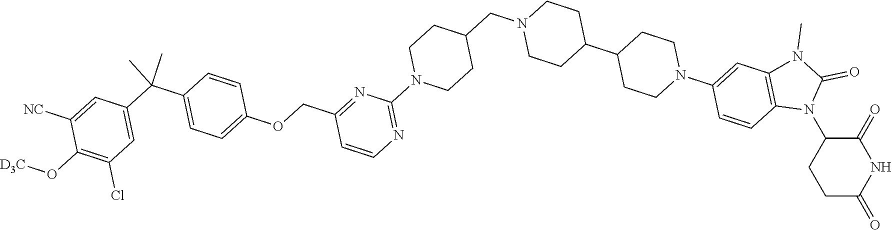 [SMILES: [2H]C([2H])([2H])Oc1c(Cl)cc(C(C)(C)c2ccc(OCc3ccnc(N4CCC(CN5CCC(C6CCN(c7ccc8c(c7)n(C)c(=O)n8C7CCC(=O)NC7=O)CC6)CC5)CC4)n3)cc2)cc1C#N]

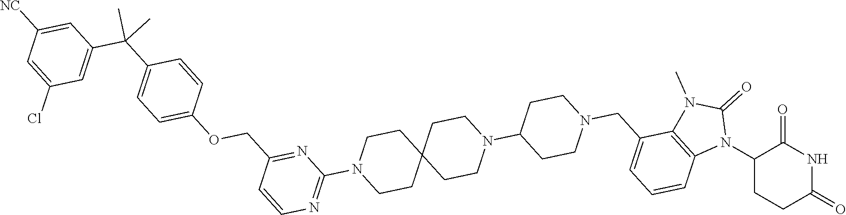 [SMILES: Cn1c(=O)n(C2CCC(=O)NC2=O)c2cccc(CN3CCC(N4CCC5(CCN(c6nccc(COc7ccc(C(C)(C)c8cc(Cl)cc(C#N)c8)cc7)n6)CC5)CC4)CC3)c21]

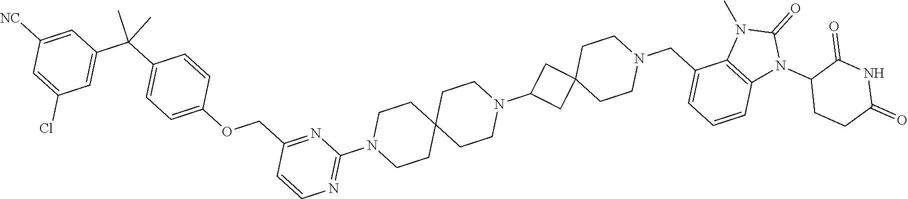 [SMILES: Cn1c(=O)n(C2CCC(=O)NC2=O)c2cccc(CN3CCC4(CC3)CC(N3CCC5(CCN(c6nccc(COc7ccc(C(C)(C)c8cc(Cl)cc(C#N)c8)cc7)n6)CC5)CC3)C4)c21]

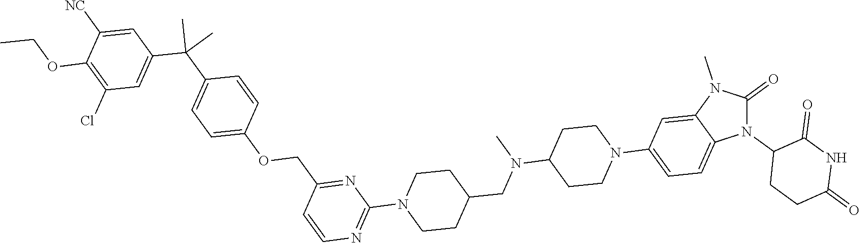 [SMILES: CCOc1c(Cl)cc(C(C)(C)c2ccc(OCc3ccnc(N4CCC(CN(C)C5CCN(c6ccc7c(c6)n(C)c(=O)n7C6CCC(=O)NC6=O)CC5)CC4)n3)cc2)cc1C#N]

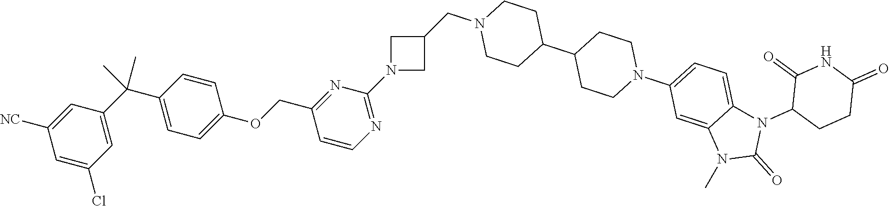 [SMILES: Cn1c(=O)n(C2CCC(=O)NC2=O)c2ccc(N3CCC(C4CCN(CC5CN(c6nccc(COc7ccc(C(C)(C)c8cc(Cl)cc(C#N)c8)cc7)n6)C5)CC4)CC3)cc21]

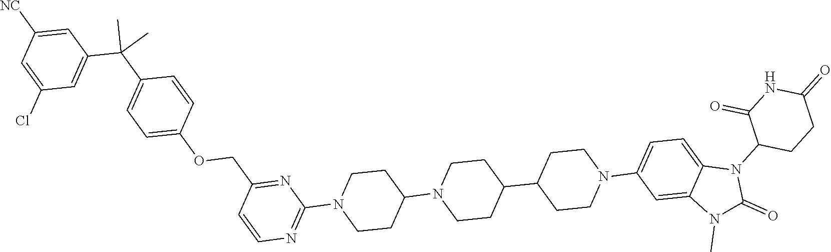 [SMILES: Cn1c(=O)n(C2CCC(=O)NC2=O)c2ccc(N3CCC(C4CCN(C5CCN(c6nccc(COc7ccc(C(C)(C)c8cc(Cl)cc(C#N)c8)cc7)n6)CC5)CC4)CC3)cc21]

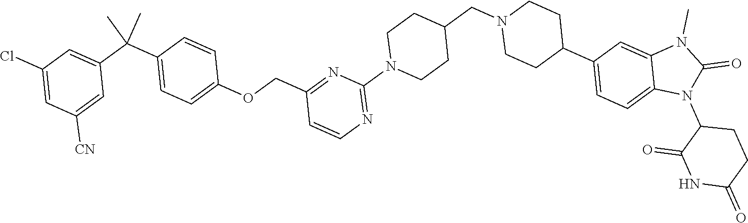 [SMILES: Cn1c(=O)n(C2CCC(=O)NC2=O)c2ccc(C3CCN(CC4CCN(c5nccc(COc6ccc(C(C)(C)c7cc(Cl)cc(C#N)c7)cc6)n5)CC4)CC3)cc21]

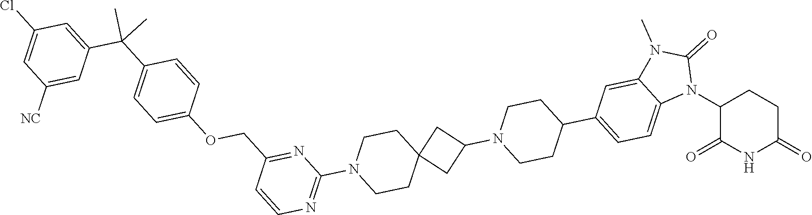 [SMILES: Cn1c(=O)n(C2CCC(=O)NC2=O)c2ccc(C3CCN(C4CC5(CCN(c6nccc(COc7ccc(C(C)(C)c8cc(Cl)cc(C#N)c8)cc7)n6)CC5)C4)CC3)cc21]

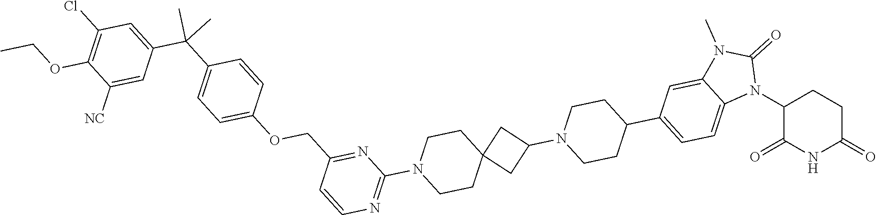 [SMILES: CCOc1c(Cl)cc(C(C)(C)c2ccc(OCc3ccnc(N4CCC5(CC4)CC(N4CCC(c6ccc7c(c6)n(C)c(=O)n7C6CCC(=O)NC6=O)CC4)C5)n3)cc2)cc1C#N]

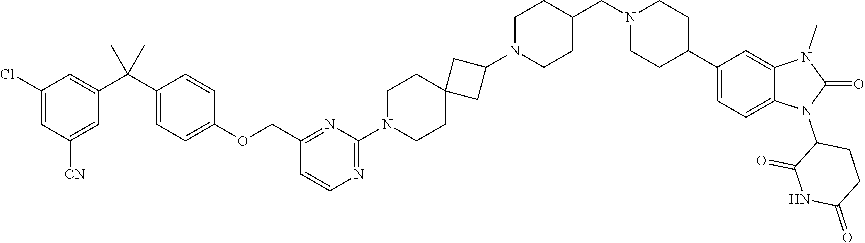 [SMILES: Cn1c(=O)n(C2CCC(=O)NC2=O)c2ccc(C3CCN(CC4CCN(C5CC6(CCN(c7nccc(COc8ccc(C(C)(C)c9cc(Cl)cc(C#N)c9)cc8)n7)CC6)C5)CC4)CC3)cc21]

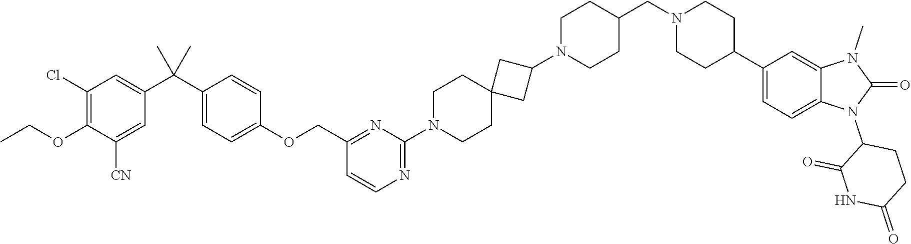 [SMILES: CCOc1c(Cl)cc(C(C)(C)c2ccc(OCc3ccnc(N4CCC5(CC4)CC(N4CCC(CN6CCC(c7ccc8c(c7)n(C)c(=O)n8C7CCC(=O)NC7=O)CC6)CC4)C5)n3)cc2)cc1C#N]

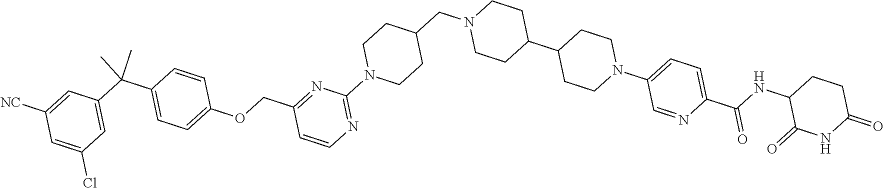 [SMILES: CC(C)(c1ccc(OCc2ccnc(N3CCC(CN4CCC(C5CCN(c6ccc(C(=O)NC7CCC(=O)NC7=O)nc6)CC5)CC4)CC3)n2)cc1)c1cc(Cl)cc(C#N)c1]

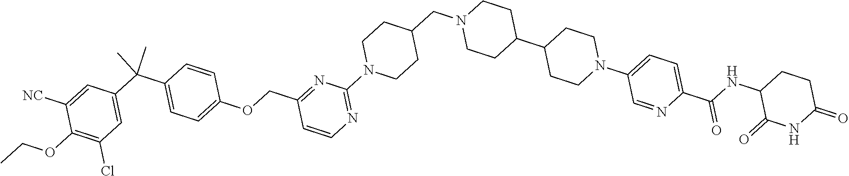 [SMILES: CCOc1c(Cl)cc(C(C)(C)c2ccc(OCc3ccnc(N4CCC(CN5CCC(C6CCN(c7ccc(C(=O)NC8CCC(=O)NC8=O)nc7)CC6)CC5)CC4)n3)cc2)cc1C#N]

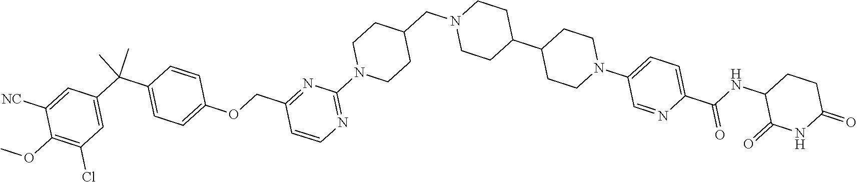 [SMILES: COc1c(Cl)cc(C(C)(C)c2ccc(OCc3ccnc(N4CCC(CN5CCC(C6CCN(c7ccc(C(=O)NC8CCC(=O)NC8=O)nc7)CC6)CC5)CC4)n3)cc2)cc1C#N]

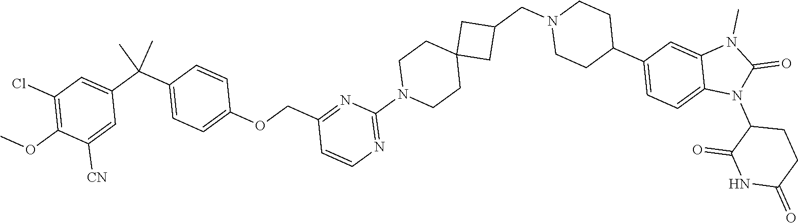 [SMILES: COc1c(Cl)cc(C(C)(C)c2ccc(OCc3ccnc(N4CCC5(CC4)CC(CN4CCC(c6ccc7c(c6)n(C)c(=O)n7C6CCC(=O)NC6=O)CC4)C5)n3)cc2)cc1C#N]

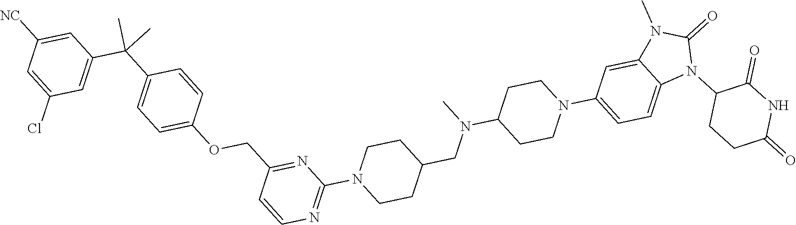 [SMILES: CN(CC1CCN(c2nccc(COc3ccc(C(C)(C)c4cc(Cl)cc(C#N)c4)cc3)n2)CC1)C1CCN(c2ccc3c(c2)n(C)c(=O)n3C2CCC(=O)NC2=O)CC1]

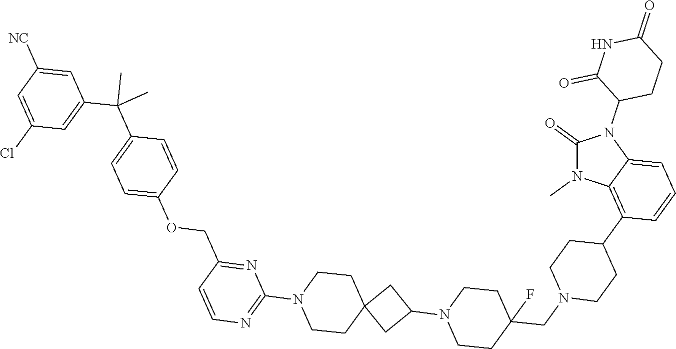 [SMILES: Cn1c(=O)n(C2CCC(=O)NC2=O)c2cccc(C3CCN(CC4(F)CCN(C5CC6(CCN(c7nccc(COc8ccc(C(C)(C)c9cc(Cl)cc(C#N)c9)cc8)n7)CC6)C5)CC4)CC3)c21]

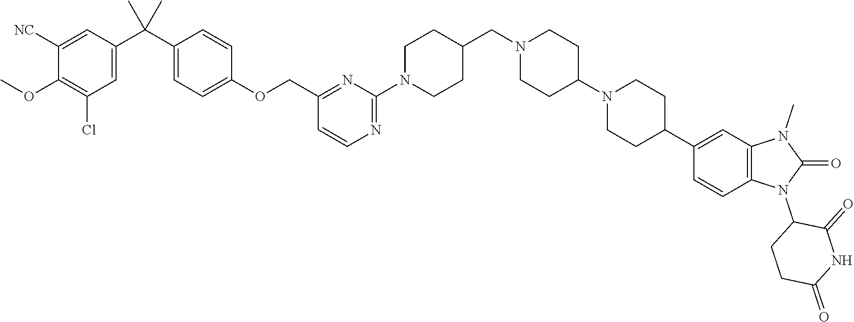 [SMILES: COc1c(Cl)cc(C(C)(C)c2ccc(OCc3ccnc(N4CCC(CN5CCC(N6CCC(c7ccc8c(c7)n(C)c(=O)n8C7CCC(=O)NC7=O)CC6)CC5)CC4)n3)cc2)cc1C#N]

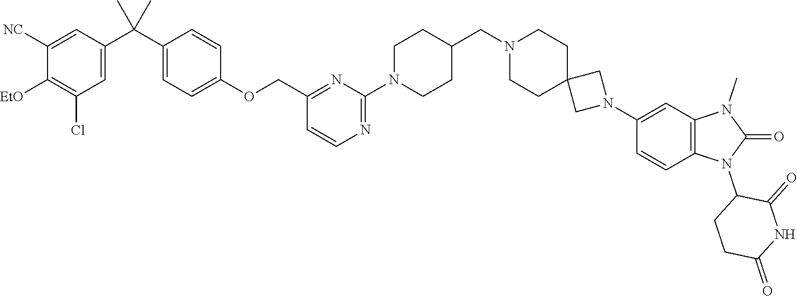 [SMILES: CCOc1c(Cl)cc(C(C)(C)c2ccc(OCc3ccnc(N4CCC(CN5CCC6(CC5)CN(c5ccc7c(c5)n(C)c(=O)n7C5CCC(=O)NC5=O)C6)CC4)n3)cc2)cc1C#N]

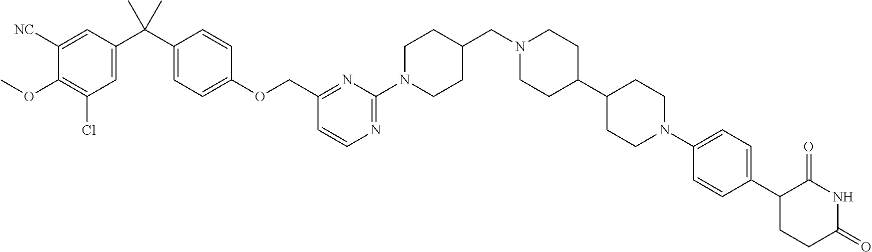 [SMILES: COc1c(Cl)cc(C(C)(C)c2ccc(OCc3ccnc(N4CCC(CN5CCC(C6CCN(c7ccc(C8CCC(=O)NC8=O)cc7)CC6)CC5)CC4)n3)cc2)cc1C#N]

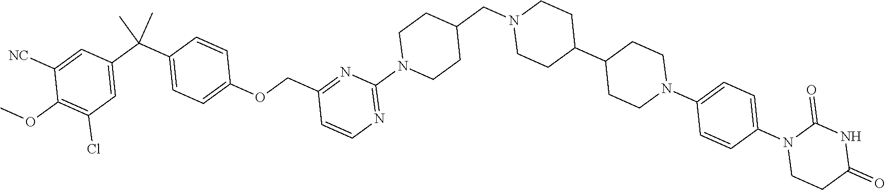 [SMILES: COc1c(Cl)cc(C(C)(C)c2ccc(OCc3ccnc(N4CCC(CN5CCC(C6CCN(c7ccc(N8CCC(=O)NC8=O)cc7)CC6)CC5)CC4)n3)cc2)cc1C#N]

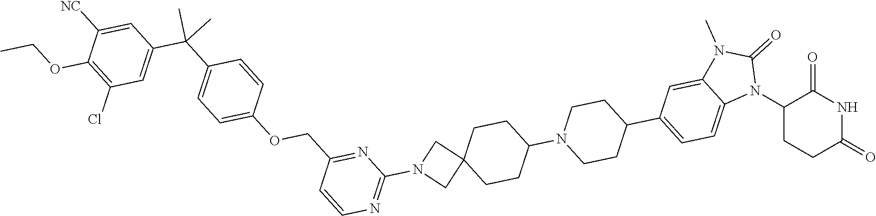 [SMILES: CCOc1c(Cl)cc(C(C)(C)c2ccc(OCc3ccnc(N4CC5(CCC(N6CCC(c7ccc8c(c7)n(C)c(=O)n8C7CCC(=O)NC7=O)CC6)CC5)C4)n3)cc2)cc1C#N]

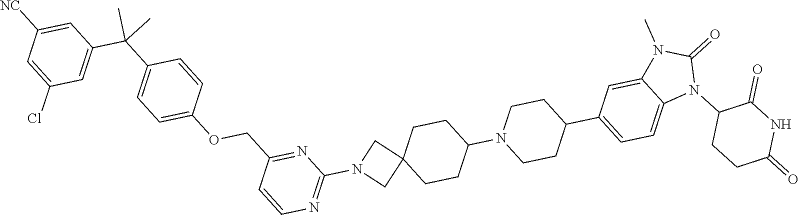 [SMILES: Cn1c(=O)n(C2CCC(=O)NC2=O)c2ccc(C3CCN(C4CCC5(CC4)CN(c4nccc(COc6ccc(C(C)(C)c7cc(Cl)cc(C#N)c7)cc6)n4)C5)CC3)cc21]

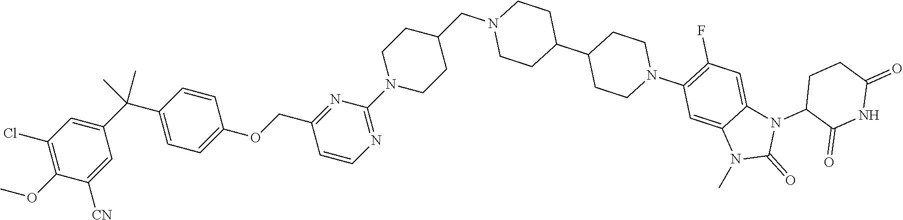 [SMILES: COc1c(Cl)cc(C(C)(C)c2ccc(OCc3ccnc(N4CCC(CN5CCC(C6CCN(c7cc8c(cc7F)n(C7CCC(=O)NC7=O)c(=O)n8C)CC6)CC5)CC4)n3)cc2)cc1C#N]

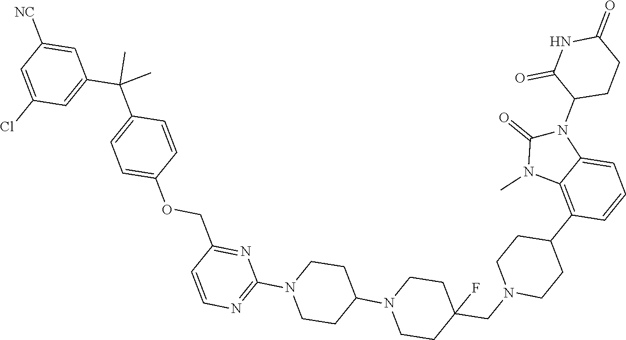 [SMILES: Cn1c(=O)n(C2CCC(=O)NC2=O)c2cccc(C3CCN(CC4(F)CCN(C5CCN(c6nccc(COc7ccc(C(C)(C)c8cc(Cl)cc(C#N)c8)cc7)n6)CC5)CC4)CC3)c21]

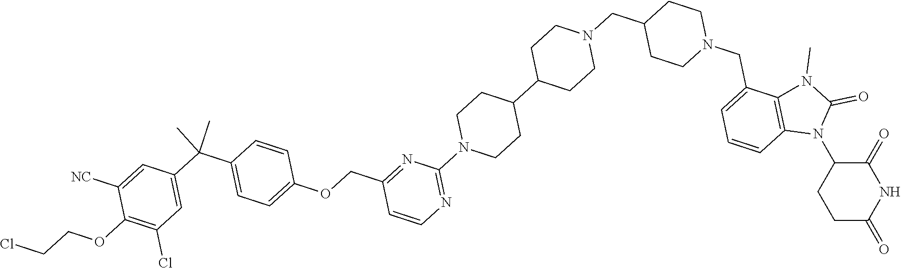 [SMILES: Cn1c(=O)n(C2CCC(=O)NC2=O)c2cccc(CN3CCC(CN4CCC(C5CCN(c6nccc(COc7ccc(C(C)(C)c8cc(Cl)c(OCCCl)c(C#N)c8)cc7)n6)CC5)CC4)CC3)c21]

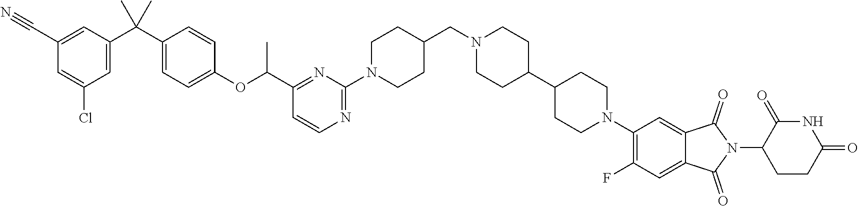 [SMILES: CC(Oc1ccc(C(C)(C)c2cc(Cl)cc(C#N)c2)cc1)c1ccnc(N2CCC(CN3CCC(C4CCN(c5cc6c(cc5F)C(=O)N(C5CCC(=O)NC5=O)C6=O)CC4)CC3)CC2)n1]

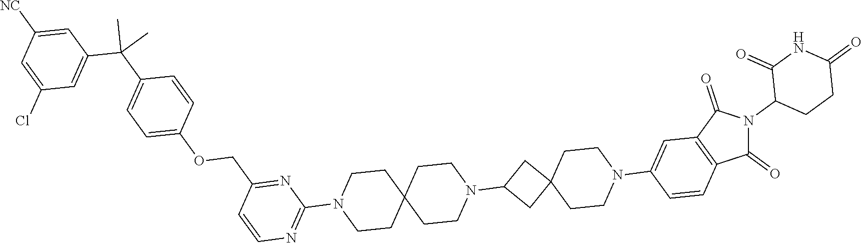 [SMILES: CC(C)(c1ccc(OCc2ccnc(N3CCC4(CC3)CCN(C3CC5(CCN(c6ccc7c(c6)C(=O)N(C6CCC(=O)NC6=O)C7=O)CC5)C3)CC4)n2)cc1)c1cc(Cl)cc(C#N)c1]

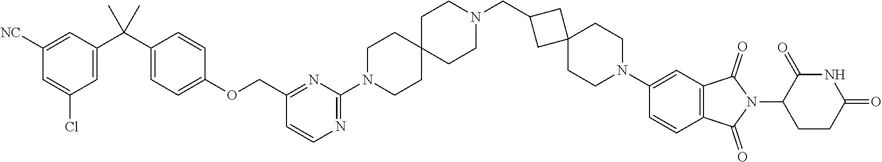 [SMILES: CC(C)(c1ccc(OCc2ccnc(N3CCC4(CCN(CC5CC6(CCN(c7ccc8c(c7)C(=O)N(C7CCC(=O)NC7=O)C8=O)CC6)C5)CC4)CC3)n2)cc1)c1cc(Cl)cc(C#N)c1]

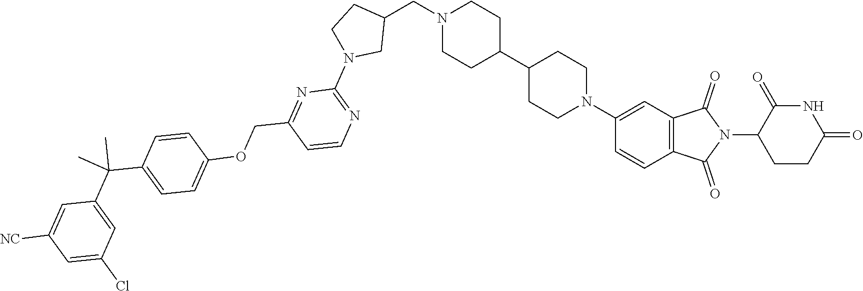 [SMILES: CC(C)(c1ccc(OCc2ccnc(N3CCC(CN4CCC(C5CCN(c6ccc7c(c6)C(=O)N(C6CCC(=O)NC6=O)C7=O)CC5)CC4)C3)n2)cc1)c1cc(Cl)cc(C#N)c1]

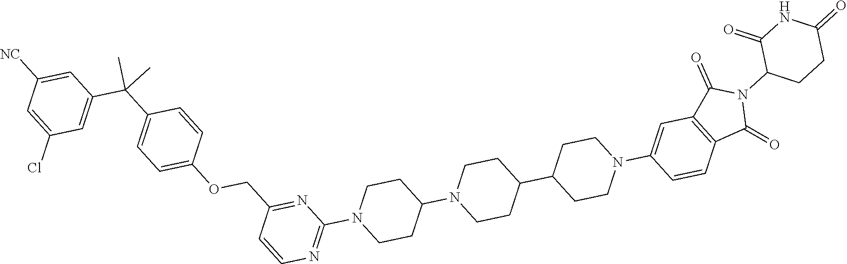 [SMILES: CC(C)(c1ccc(OCc2ccnc(N3CCC(N4CCC(C5CCN(c6ccc7c(c6)C(=O)N(C6CCC(=O)NC6=O)C7=O)CC5)CC4)CC3)n2)cc1)c1cc(Cl)cc(C#N)c1]

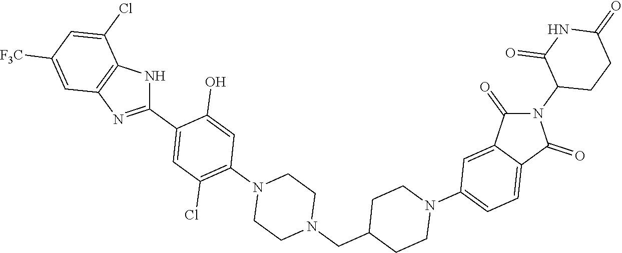 [SMILES: O=C1CCC(N2C(=O)c3ccc(N4CCC(CN5CCN(c6cc(O)c(-c7nc8cc(C(F)(F)F)cc(Cl)c8[nH]7)cc6Cl)CC5)CC4)cc3C2=O)C(=O)N1]

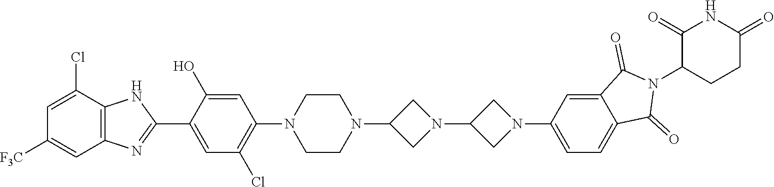 [SMILES: O=C1CCC(N2C(=O)c3ccc(N4CC(N5CC(N6CCN(c7cc(O)c(-c8nc9cc(C(F)(F)F)cc(Cl)c9[nH]8)cc7Cl)CC6)C5)C4)cc3C2=O)C(=O)N1]

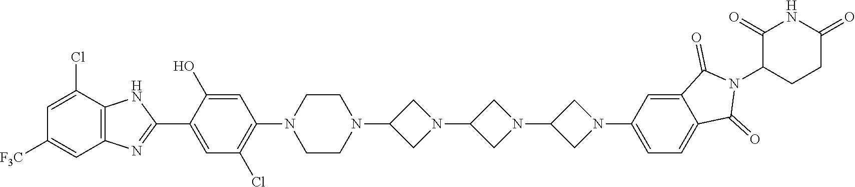 [SMILES: O=C1CCC(N2C(=O)c3ccc(N4CC(N5CC(N6CC(N7CCN(c8cc(O)c(-c9nc%10cc(C(F)(F)F)cc(Cl)c%10[nH]9)cc8Cl)CC7)C6)C5)C4)cc3C2=O)C(=O)N1]

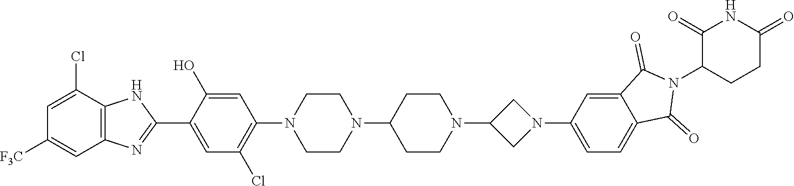 [SMILES: O=C1CCC(N2C(=O)c3ccc(N4CC(N5CCC(N6CCN(c7cc(O)c(-c8nc9cc(C(F)(F)F)cc(Cl)c9[nH]8)cc7Cl)CC6)CC5)C4)cc3C2=O)C(=O)N1]

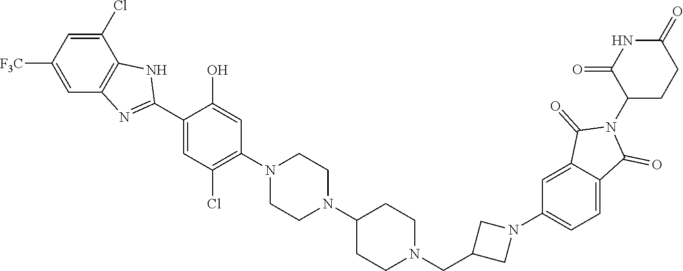 [SMILES: O=C1CCC(N2C(=O)c3ccc(N4CC(CN5CCC(N6CCN(c7cc(O)c(-c8nc9cc(C(F)(F)F)cc(Cl)c9[nH]8)cc7Cl)CC6)CC5)C4)cc3C2=O)C(=O)N1]

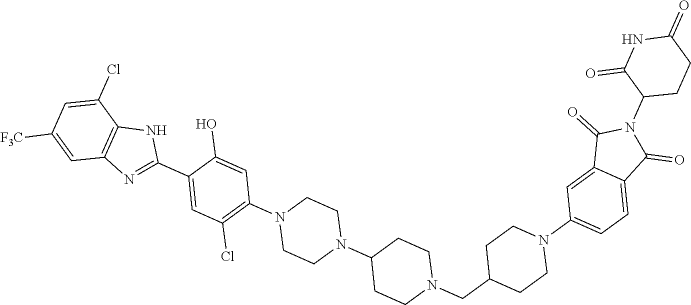 [SMILES: O=C1CCC(N2C(=O)c3ccc(N4CCC(CN5CCC(N6CCN(c7cc(O)c(-c8nc9cc(C(F)(F)F)cc(Cl)c9[nH]8)cc7Cl)CC6)CC5)CC4)cc3C2=O)C(=O)N1]

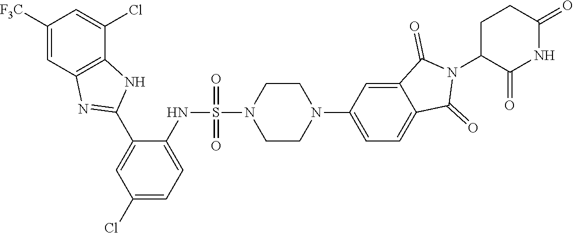 [SMILES: O=C1CCC(N2C(=O)c3ccc(N4CCN(S(=O)(=O)Nc5ccc(Cl)cc5-c5nc6cc(C(F)(F)F)cc(Cl)c6[nH]5)CC4)cc3C2=O)C(=O)N1]

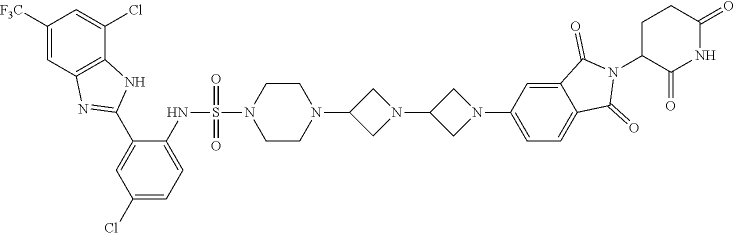 [SMILES: O=C1CCC(N2C(=O)c3ccc(N4CC(N5CC(N6CCN(S(=O)(=O)Nc7ccc(Cl)cc7-c7nc8cc(C(F)(F)F)cc(Cl)c8[nH]7)CC6)C5)C4)cc3C2=O)C(=O)N1]

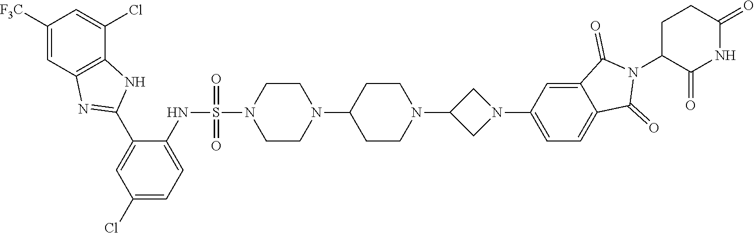 [SMILES: O=C1CCC(N2C(=O)c3ccc(N4CC(N5CCC(N6CCN(S(=O)(=O)Nc7ccc(Cl)cc7-c7nc8cc(C(F)(F)F)cc(Cl)c8[nH]7)CC6)CC5)C4)cc3C2=O)C(=O)N1]

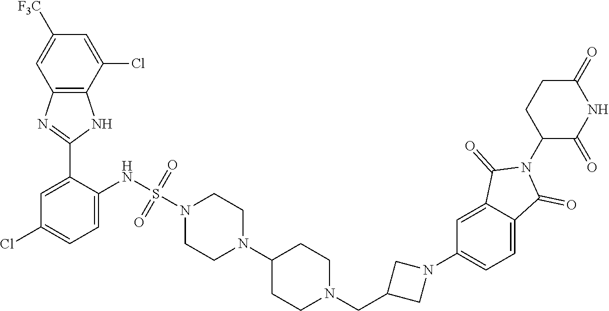 [SMILES: O=C1CCC(N2C(=O)c3ccc(N4CC(CN5CCC(N6CCN(S(=O)(=O)Nc7ccc(Cl)cc7-c7nc8cc(C(F)(F)F)cc(Cl)c8[nH]7)CC6)CC5)C4)cc3C2=O)C(=O)N1]

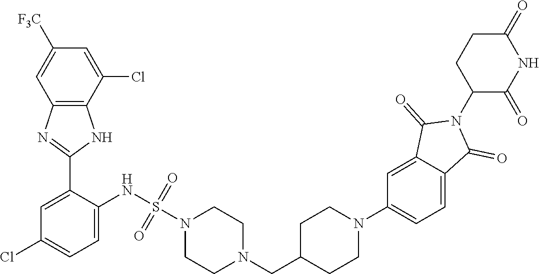 [SMILES: O=C1CCC(N2C(=O)c3ccc(N4CCC(CN5CCN(S(=O)(=O)Nc6ccc(Cl)cc6-c6nc7cc(C(F)(F)F)cc(Cl)c7[nH]6)CC5)CC4)cc3C2=O)C(=O)N1]

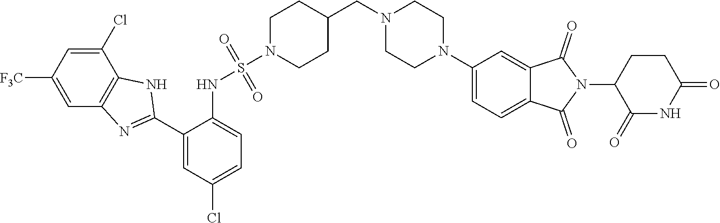 [SMILES: O=C1CCC(N2C(=O)c3ccc(N4CCN(CC5CCN(S(=O)(=O)Nc6ccc(Cl)cc6-c6nc7cc(C(F)(F)F)cc(Cl)c7[nH]6)CC5)CC4)cc3C2=O)C(=O)N1]

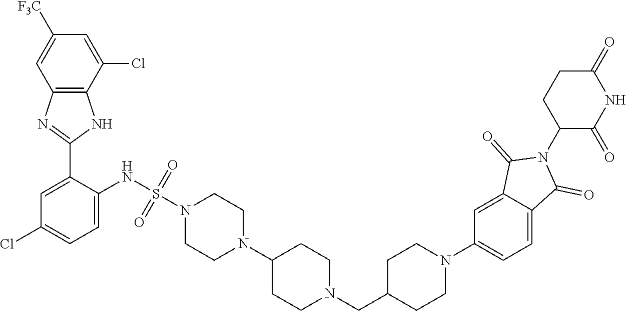 [SMILES: O=C1CCC(N2C(=O)c3ccc(N4CCC(CN5CCC(N6CCN(S(=O)(=O)Nc7ccc(Cl)cc7-c7nc8cc(C(F)(F)F)cc(Cl)c8[nH]7)CC6)CC5)CC4)cc3C2=O)C(=O)N1]

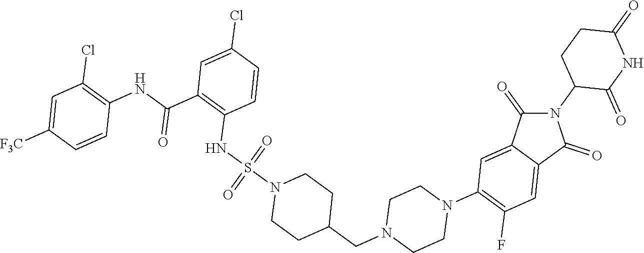 [SMILES: O=C1CCC(N2C(=O)c3cc(F)c(N4CCN(CC5CCN(S(=O)(=O)Nc6ccc(Cl)cc6C(=O)Nc6ccc(C(F)(F)F)cc6Cl)CC5)CC4)cc3C2=O)C(=O)N1]

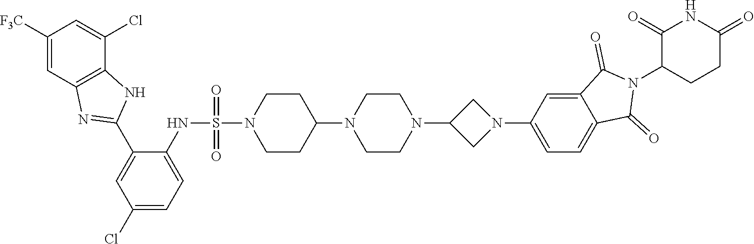 [SMILES: O=C1CCC(N2C(=O)c3ccc(N4CC(N5CCN(C6CCN(S(=O)(=O)Nc7ccc(Cl)cc7-c7nc8cc(C(F)(F)F)cc(Cl)c8[nH]7)CC6)CC5)C4)cc3C2=O)C(=O)N1]

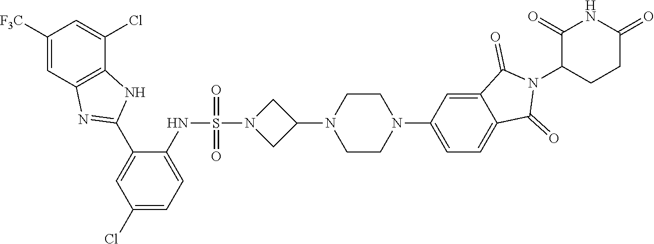 [SMILES: O=C1CCC(N2C(=O)c3ccc(N4CCN(C5CN(S(=O)(=O)Nc6ccc(Cl)cc6-c6nc7cc(C(F)(F)F)cc(Cl)c7[nH]6)C5)CC4)cc3C2=O)C(=O)N1]

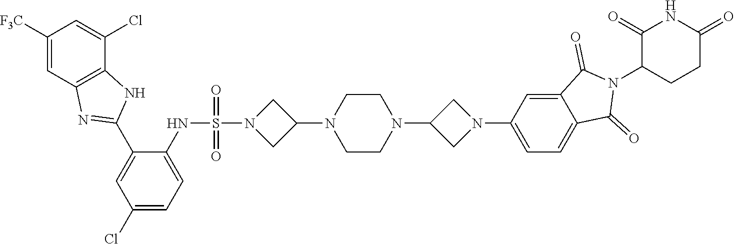 [SMILES: O=C1CCC(N2C(=O)c3ccc(N4CC(N5CCN(C6CN(S(=O)(=O)Nc7ccc(Cl)cc7-c7nc8cc(C(F)(F)F)cc(Cl)c8[nH]7)C6)CC5)C4)cc3C2=O)C(=O)N1]

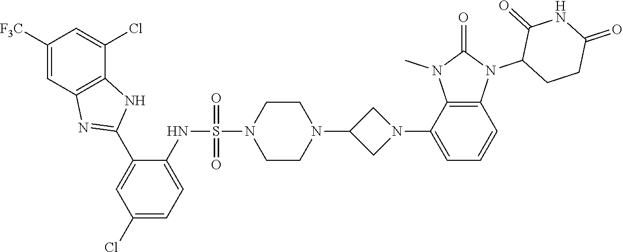 [SMILES: Cn1c(=O)n(C2CCC(=O)NC2=O)c2cccc(N3CC(N4CCN(S(=O)(=O)Nc5ccc(Cl)cc5-c5nc6cc(C(F)(F)F)cc(Cl)c6[nH]5)CC4)C3)c21]

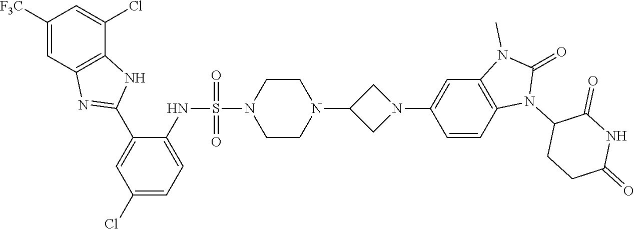 [SMILES: Cn1c(=O)n(C2CCC(=O)NC2=O)c2ccc(N3CC(N4CCN(S(=O)(=O)Nc5ccc(Cl)cc5-c5nc6cc(C(F)(F)F)cc(Cl)c6[nH]5)CC4)C3)cc21]

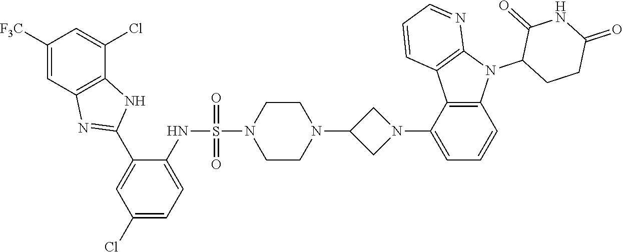 [SMILES: O=C1CCC(n2c3cccc(N4CC(N5CCN(S(=O)(=O)Nc6ccc(Cl)cc6-c6nc7cc(C(F)(F)F)cc(Cl)c7[nH]6)CC5)C4)c3c3cccnc32)C(=O)N1]

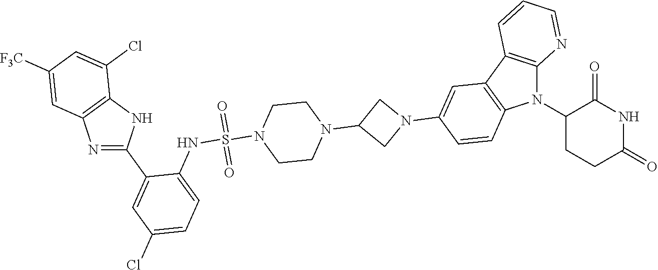 [SMILES: O=C1CCC(n2c3ccc(N4CC(N5CCN(S(=O)(=O)Nc6ccc(Cl)cc6-c6nc7cc(C(F)(F)F)cc(Cl)c7[nH]6)CC5)C4)cc3c3cccnc32)C(=O)N1]

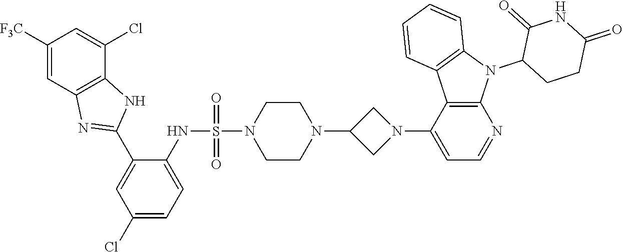 [SMILES: O=C1CCC(n2c3ccccc3c3c(N4CC(N5CCN(S(=O)(=O)Nc6ccc(Cl)cc6-c6nc7cc(C(F)(F)F)cc(Cl)c7[nH]6)CC5)C4)ccnc32)C(=O)N1]

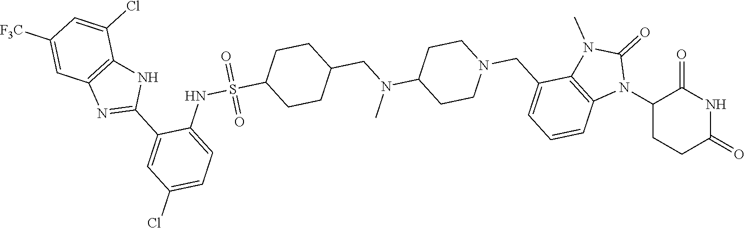 [SMILES: CN(CC1CCC(S(=O)(=O)Nc2ccc(Cl)cc2-c2nc3cc(C(F)(F)F)cc(Cl)c3[nH]2)CC1)C1CCN(Cc2cccc3c2n(C)c(=O)n3C2CCC(=O)NC2=O)CC1]

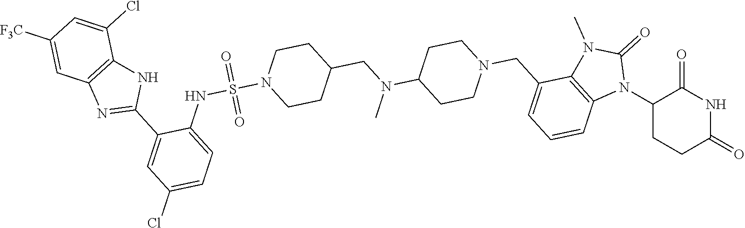 [SMILES: CN(CC1CCN(S(=O)(=O)Nc2ccc(Cl)cc2-c2nc3cc(C(F)(F)F)cc(Cl)c3[nH]2)CC1)C1CCN(Cc2cccc3c2n(C)c(=O)n3C2CCC(=O)NC2=O)CC1]

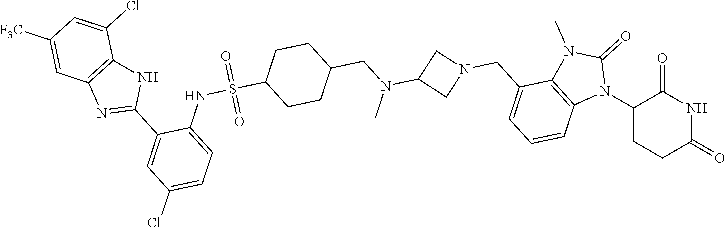 [SMILES: CN(CC1CCC(S(=O)(=O)Nc2ccc(Cl)cc2-c2nc3cc(C(F)(F)F)cc(Cl)c3[nH]2)CC1)C1CN(Cc2cccc3c2n(C)c(=O)n3C2CCC(=O)NC2=O)C1]

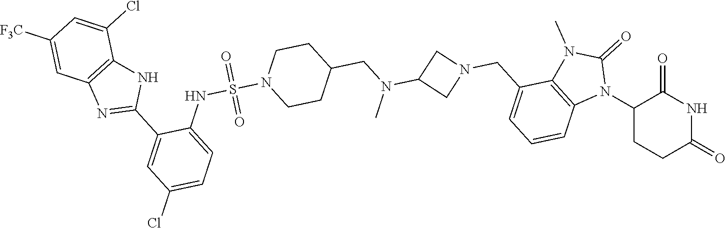 [SMILES: CN(CC1CCN(S(=O)(=O)Nc2ccc(Cl)cc2-c2nc3cc(C(F)(F)F)cc(Cl)c3[nH]2)CC1)C1CN(Cc2cccc3c2n(C)c(=O)n3C2CCC(=O)NC2=O)C1]

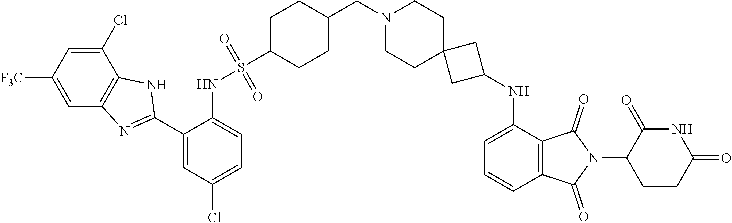 [SMILES: O=C1CCC(N2C(=O)c3cccc(NC4CC5(CCN(CC6CCC(S(=O)(=O)Nc7ccc(Cl)cc7-c7nc8cc(C(F)(F)F)cc(Cl)c8[nH]7)CC6)CC5)C4)c3C2=O)C(=O)N1]

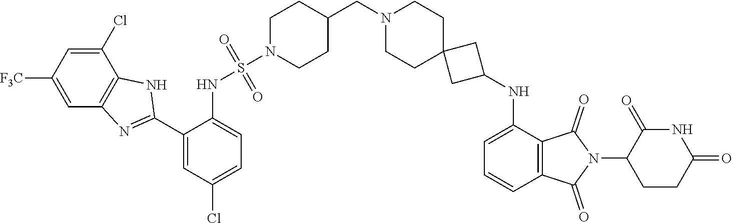 [SMILES: O=C1CCC(N2C(=O)c3cccc(NC4CC5(CCN(CC6CCN(S(=O)(=O)Nc7ccc(Cl)cc7-c7nc8cc(C(F)(F)F)cc(Cl)c8[nH]7)CC6)CC5)C4)c3C2=O)C(=O)N1]

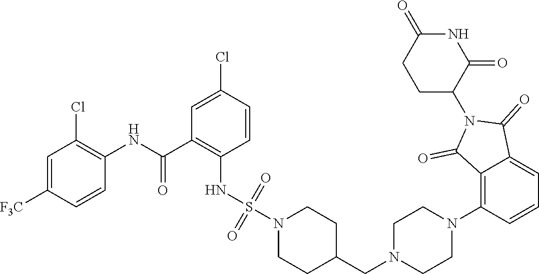 [SMILES: O=C1CCC(N2C(=O)c3cccc(N4CCN(CC5CCN(S(=O)(=O)Nc6ccc(Cl)cc6C(=O)Nc6ccc(C(F)(F)F)cc6Cl)CC5)CC4)c3C2=O)C(=O)N1]

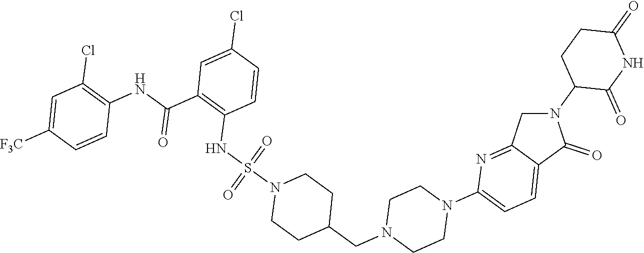 [SMILES: O=C1CCC(N2Cc3nc(N4CCN(CC5CCN(S(=O)(=O)Nc6ccc(Cl)cc6C(=O)Nc6ccc(C(F)(F)F)cc6Cl)CC5)CC4)ccc3C2=O)C(=O)N1]

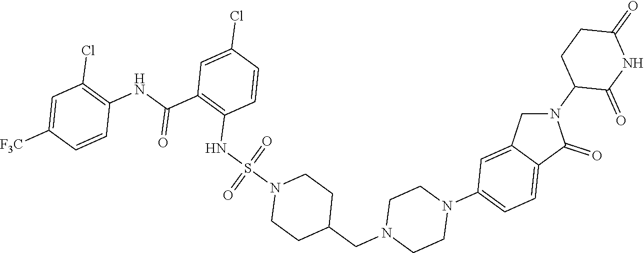 [SMILES: O=C1CCC(N2Cc3cc(N4CCN(CC5CCN(S(=O)(=O)Nc6ccc(Cl)cc6C(=O)Nc6ccc(C(F)(F)F)cc6Cl)CC5)CC4)ccc3C2=O)C(=O)N1]

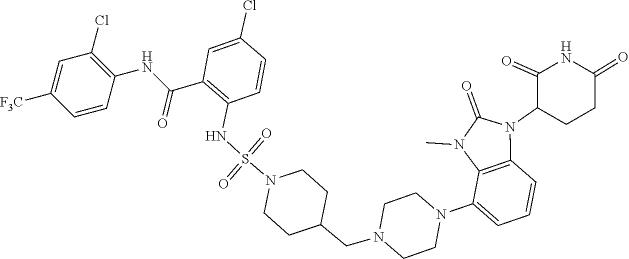 [SMILES: Cn1c(=O)n(C2CCC(=O)NC2=O)c2cccc(N3CCN(CC4CCN(S(=O)(=O)Nc5ccc(Cl)cc5C(=O)Nc5ccc(C(F)(F)F)cc5Cl)CC4)CC3)c21]

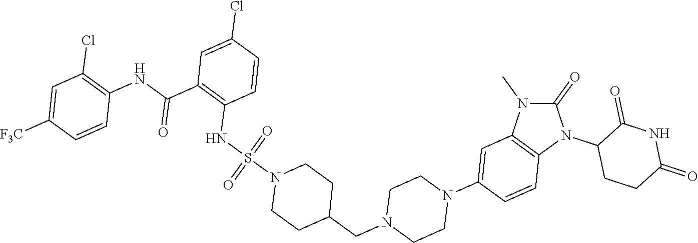 [SMILES: Cn1c(=O)n(C2CCC(=O)NC2=O)c2ccc(N3CCN(CC4CCN(S(=O)(=O)Nc5ccc(Cl)cc5C(=O)Nc5ccc(C(F)(F)F)cc5Cl)CC4)CC3)cc21]

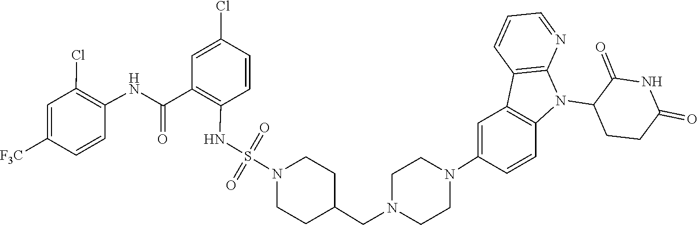 [SMILES: O=C1CCC(n2c3ccc(N4CCN(CC5CCN(S(=O)(=O)Nc6ccc(Cl)cc6C(=O)Nc6ccc(C(F)(F)F)cc6Cl)CC5)CC4)cc3c3cccnc32)C(=O)N1]

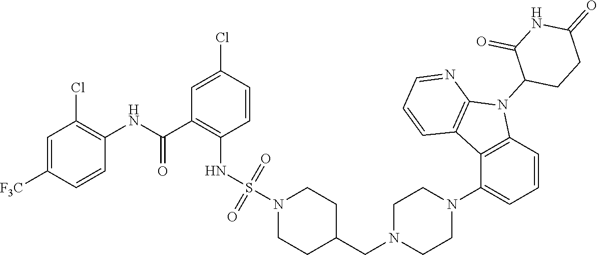 [SMILES: O=C1CCC(n2c3cccc(N4CCN(CC5CCN(S(=O)(=O)Nc6ccc(Cl)cc6C(=O)Nc6ccc(C(F)(F)F)cc6Cl)CC5)CC4)c3c3cccnc32)C(=O)N1]

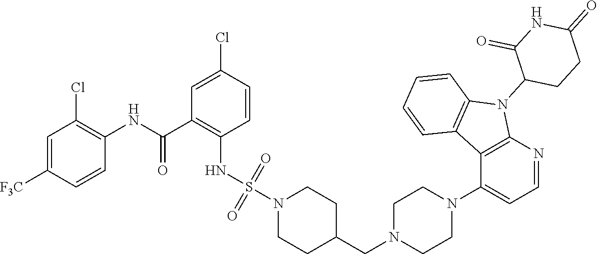 [SMILES: O=C1CCC(n2c3ccccc3c3c(N4CCN(CC5CCN(S(=O)(=O)Nc6ccc(Cl)cc6C(=O)Nc6ccc(C(F)(F)F)cc6Cl)CC5)CC4)ccnc32)C(=O)N1]

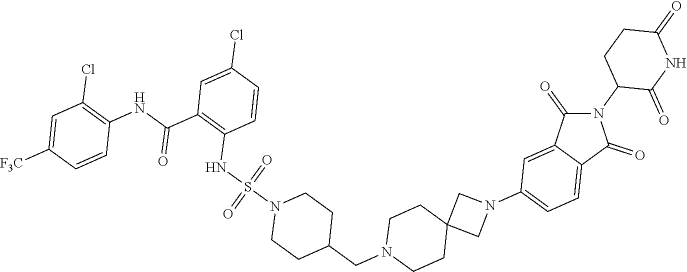 [SMILES: O=C1CCC(N2C(=O)c3ccc(N4CC5(CCN(CC6CCN(S(=O)(=O)Nc7ccc(Cl)cc7C(=O)Nc7ccc(C(F)(F)F)cc7Cl)CC6)CC5)C4)cc3C2=O)C(=O)N1]

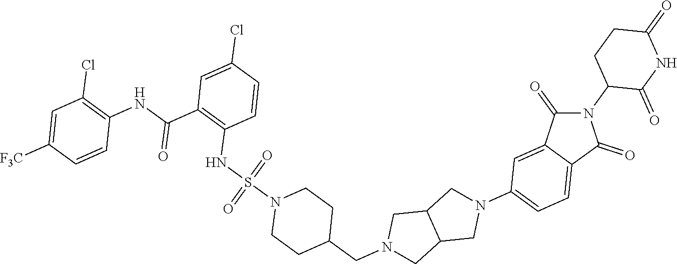 [SMILES: O=C1CCC(N2C(=O)c3ccc(N4CC5CN(CC6CCN(S(=O)(=O)Nc7ccc(Cl)cc7C(=O)Nc7ccc(C(F)(F)F)cc7Cl)CC6)CC5C4)cc3C2=O)C(=O)N1]

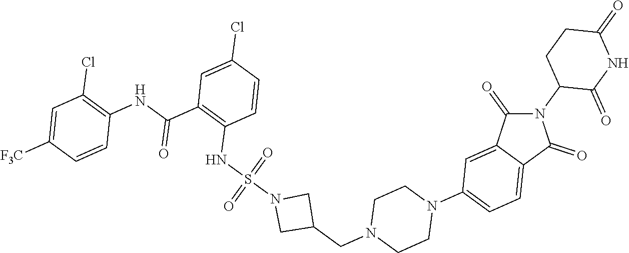 [SMILES: O=C1CCC(N2C(=O)c3ccc(N4CCN(CC5CN(S(=O)(=O)Nc6ccc(Cl)cc6C(=O)Nc6ccc(C(F)(F)F)cc6Cl)C5)CC4)cc3C2=O)C(=O)N1]

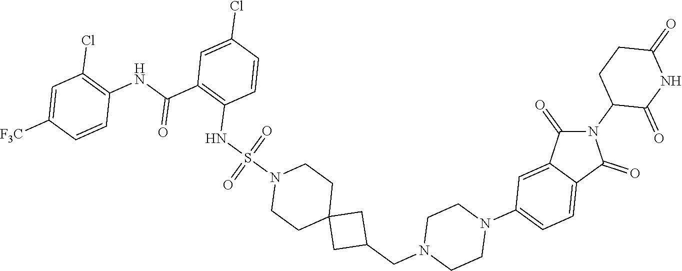 [SMILES: O=C1CCC(N2C(=O)c3ccc(N4CCN(CC5CC6(CCN(S(=O)(=O)Nc7ccc(Cl)cc7C(=O)Nc7ccc(C(F)(F)F)cc7Cl)CC6)C5)CC4)cc3C2=O)C(=O)N1]

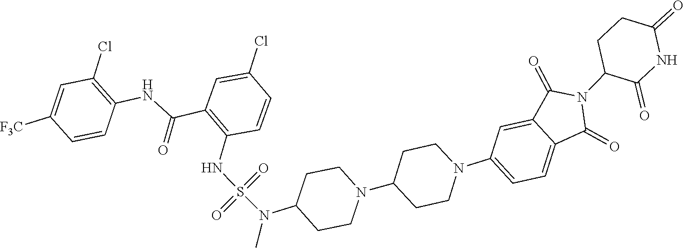 [SMILES: CN(C1CCN(C2CCN(c3ccc4c(c3)C(=O)N(C3CCC(=O)NC3=O)C4=O)CC2)CC1)S(=O)(=O)Nc1ccc(Cl)cc1C(=O)Nc1ccc(C(F)(F)F)cc1Cl]

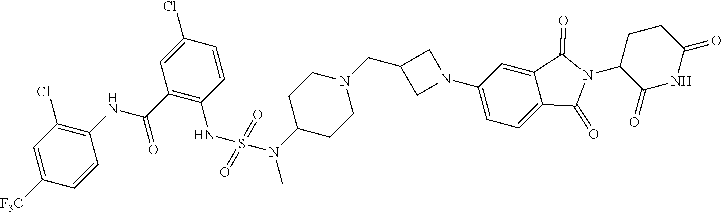 [SMILES: CN(C1CCN(CC2CN(c3ccc4c(c3)C(=O)N(C3CCC(=O)NC3=O)C4=O)C2)CC1)S(=O)(=O)Nc1ccc(Cl)cc1C(=O)Nc1ccc(C(F)(F)F)cc1Cl]

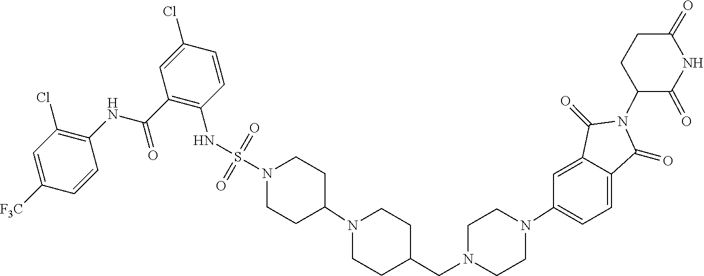 [SMILES: O=C1CCC(N2C(=O)c3ccc(N4CCN(CC5CCN(C6CCN(S(=O)(=O)Nc7ccc(Cl)cc7C(=O)Nc7ccc(C(F)(F)F)cc7Cl)CC6)CC5)CC4)cc3C2=O)C(=O)N1]

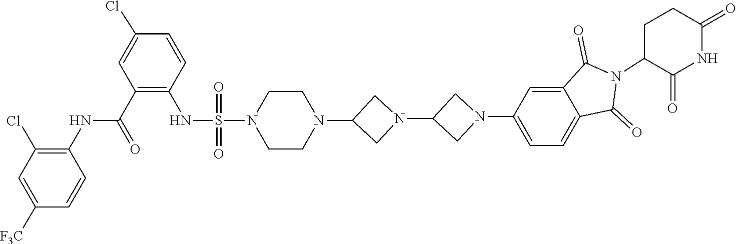 [SMILES: O=C1CCC(N2C(=O)c3ccc(N4CC(N5CC(N6CCN(S(=O)(=O)Nc7ccc(Cl)cc7C(=O)Nc7ccc(C(F)(F)F)cc7Cl)CC6)C5)C4)cc3C2=O)C(=O)N1]

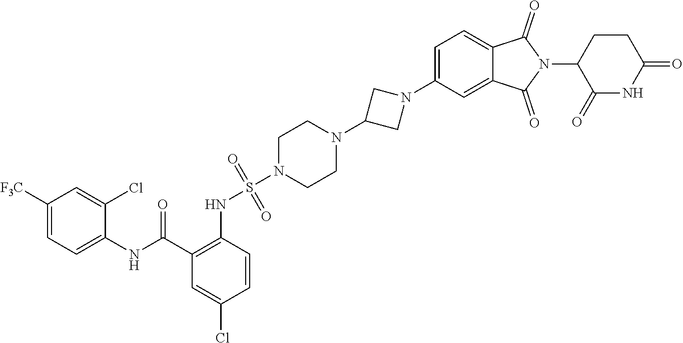 [SMILES: O=C1CCC(N2C(=O)c3ccc(N4CC(N5CCN(S(=O)(=O)Nc6ccc(Cl)cc6C(=O)Nc6ccc(C(F)(F)F)cc6Cl)CC5)C4)cc3C2=O)C(=O)N1]

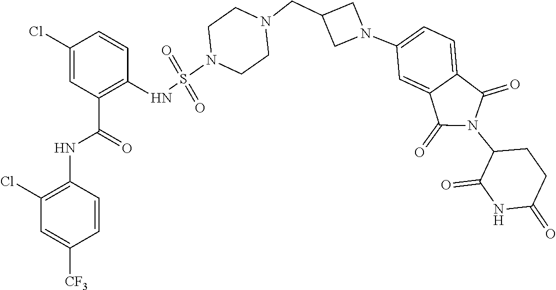 [SMILES: O=C1CCC(N2C(=O)c3ccc(N4CC(CN5CCN(S(=O)(=O)Nc6ccc(Cl)cc6C(=O)Nc6ccc(C(F)(F)F)cc6Cl)CC5)C4)cc3C2=O)C(=O)N1]

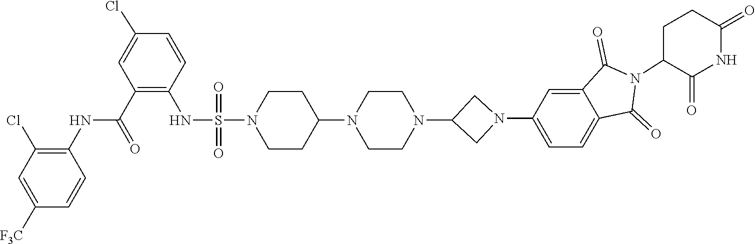 [SMILES: O=C1CCC(N2C(=O)c3ccc(N4CC(N5CCN(C6CCN(S(=O)(=O)Nc7ccc(Cl)cc7C(=O)Nc7ccc(C(F)(F)F)cc7Cl)CC6)CC5)C4)cc3C2=O)C(=O)N1]

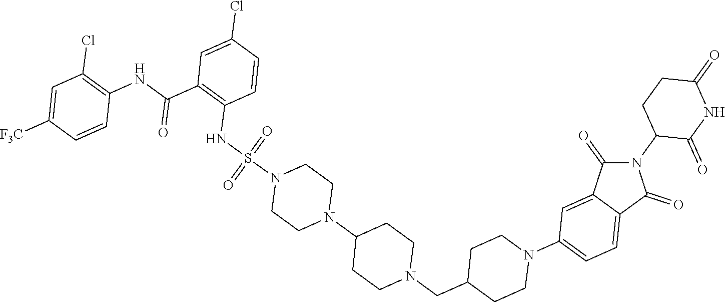 [SMILES: O=C1CCC(N2C(=O)c3ccc(N4CCC(CN5CCC(N6CCN(S(=O)(=O)Nc7ccc(Cl)cc7C(=O)Nc7ccc(C(F)(F)F)cc7Cl)CC6)CC5)CC4)cc3C2=O)C(=O)N1]

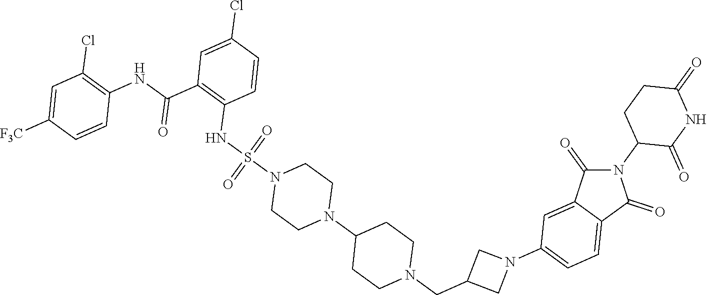 [SMILES: O=C1CCC(N2C(=O)c3ccc(N4CC(CN5CCC(N6CCN(S(=O)(=O)Nc7ccc(Cl)cc7C(=O)Nc7ccc(C(F)(F)F)cc7Cl)CC6)CC5)C4)cc3C2=O)C(=O)N1]

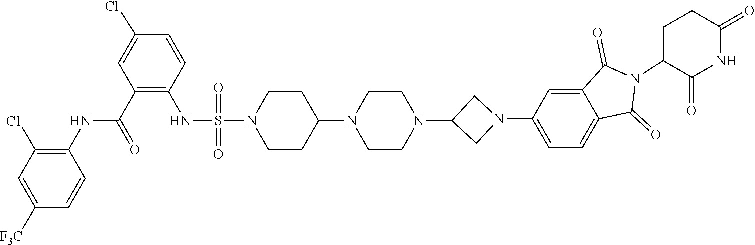 [SMILES: O=C1CCC(N2C(=O)c3ccc(N4CC(N5CCN(C6CCN(S(=O)(=O)Nc7ccc(Cl)cc7C(=O)Nc7ccc(C(F)(F)F)cc7Cl)CC6)CC5)C4)cc3C2=O)C(=O)N1]

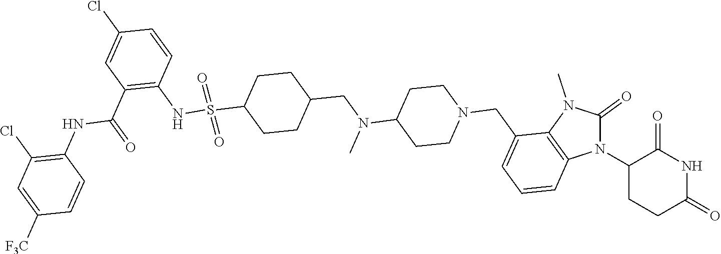 [SMILES: CN(CC1CCC(S(=O)(=O)Nc2ccc(Cl)cc2C(=O)Nc2ccc(C(F)(F)F)cc2Cl)CC1)C1CCN(Cc2cccc3c2n(C)c(=O)n3C2CCC(=O)NC2=O)CC1]

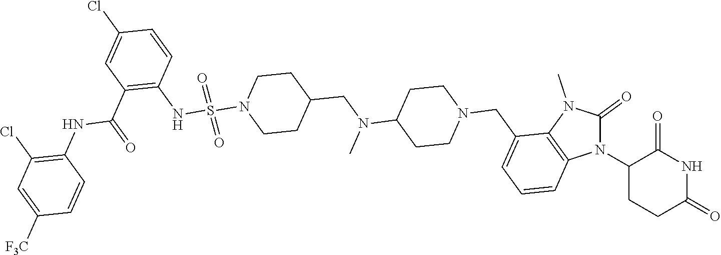 [SMILES: CN(CC1CCN(S(=O)(=O)Nc2ccc(Cl)cc2C(=O)Nc2ccc(C(F)(F)F)cc2Cl)CC1)C1CCN(Cc2cccc3c2n(C)c(=O)n3C2CCC(=O)NC2=O)CC1]

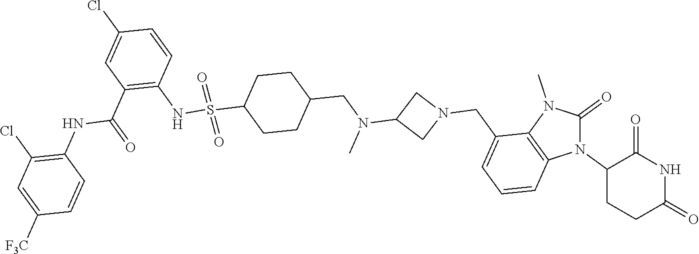 [SMILES: CN(CC1CCC(S(=O)(=O)Nc2ccc(Cl)cc2C(=O)Nc2ccc(C(F)(F)F)cc2Cl)CC1)C1CN(Cc2cccc3c2n(C)c(=O)n3C2CCC(=O)NC2=O)C1]

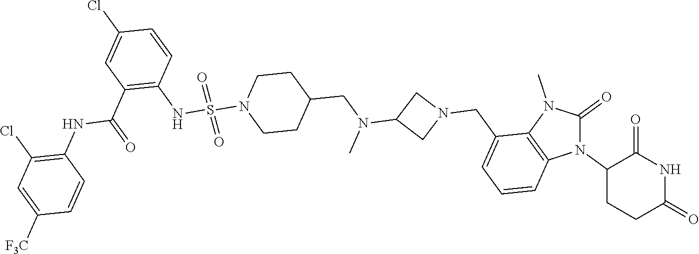 [SMILES: CN(CC1CCN(S(=O)(=O)Nc2ccc(Cl)cc2C(=O)Nc2ccc(C(F)(F)F)cc2Cl)CC1)C1CN(Cc2cccc3c2n(C)c(=O)n3C2CCC(=O)NC2=O)C1]

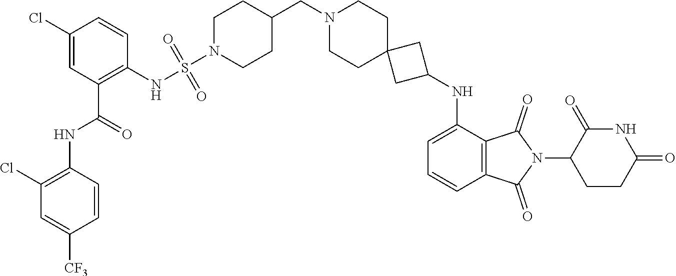 [SMILES: O=C1CCC(N2C(=O)c3cccc(NC4CC5(CCN(CC6CCN(S(=O)(=O)Nc7ccc(Cl)cc7C(=O)Nc7ccc(C(F)(F)F)cc7Cl)CC6)CC5)C4)c3C2=O)C(=O)N1]

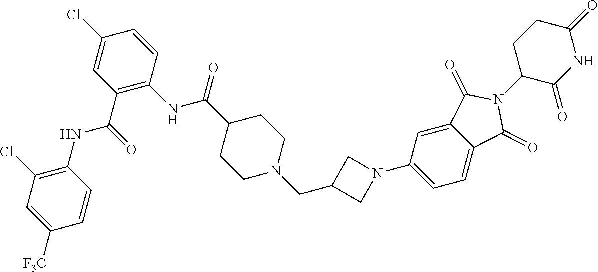 [SMILES: O=C1CCC(N2C(=O)c3ccc(N4CC(CN5CCC(C(=O)Nc6ccc(Cl)cc6C(=O)Nc6ccc(C(F)(F)F)cc6Cl)CC5)C4)cc3C2=O)C(=O)N1]

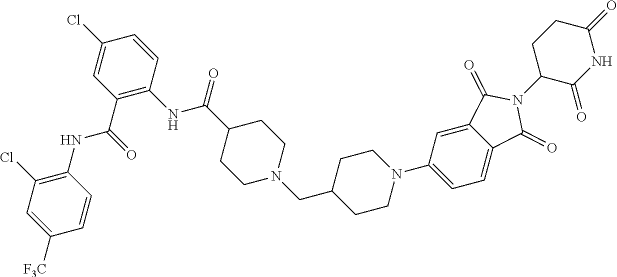 [SMILES: O=C1CCC(N2C(=O)c3ccc(N4CCC(CN5CCC(C(=O)Nc6ccc(Cl)cc6C(=O)Nc6ccc(C(F)(F)F)cc6Cl)CC5)CC4)cc3C2=O)C(=O)N1]

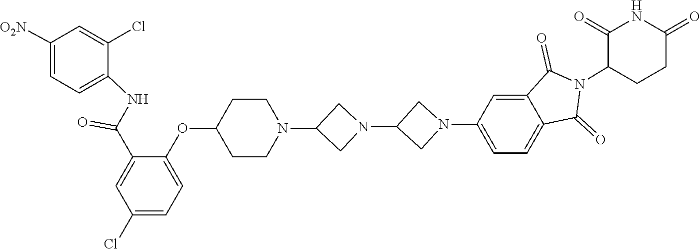 [SMILES: O=C1CCC(N2C(=O)c3ccc(N4CC(N5CC(N6CCC(Oc7ccc(Cl)cc7C(=O)Nc7ccc([N+](=O)[O-])cc7Cl)CC6)C5)C4)cc3C2=O)C(=O)N1]

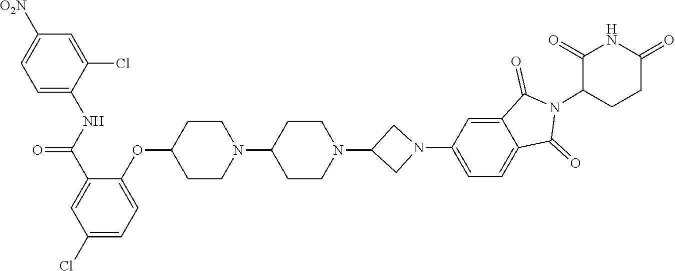 [SMILES: O=C1CCC(N2C(=O)c3ccc(N4CC(N5CCC(N6CCC(Oc7ccc(Cl)cc7C(=O)Nc7ccc([N+](=O)[O-])cc7Cl)CC6)CC5)C4)cc3C2=O)C(=O)N1]

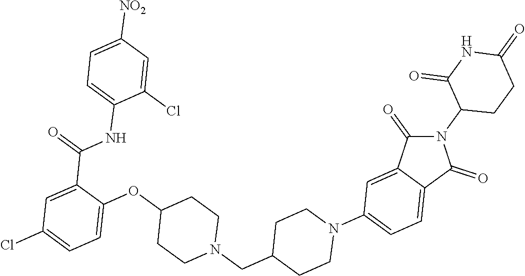 [SMILES: O=C1CCC(N2C(=O)c3ccc(N4CCC(CN5CCC(Oc6ccc(Cl)cc6C(=O)Nc6ccc([N+](=O)[O-])cc6Cl)CC5)CC4)cc3C2=O)C(=O)N1]

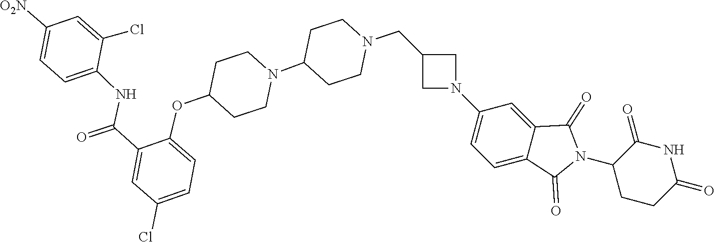 [SMILES: O=C1CCC(N2C(=O)c3ccc(N4CC(CN5CCC(N6CCC(Oc7ccc(Cl)cc7C(=O)Nc7ccc([N+](=O)[O-])cc7Cl)CC6)CC5)C4)cc3C2=O)C(=O)N1]

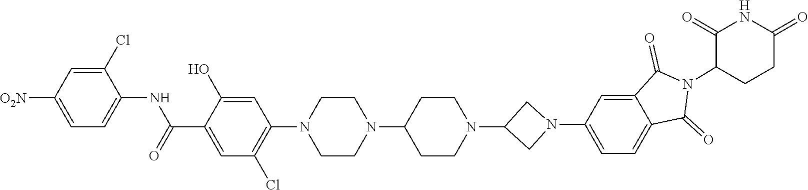 [SMILES: O=C1CCC(N2C(=O)c3ccc(N4CC(N5CCC(N6CCN(c7cc(O)c(C(=O)Nc8ccc([N+](=O)[O-])cc8Cl)cc7Cl)CC6)CC5)C4)cc3C2=O)C(=O)N1]

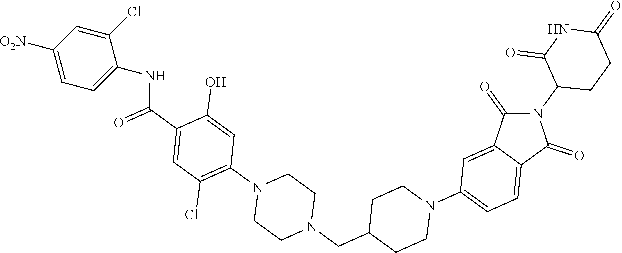 [SMILES: O=C1CCC(N2C(=O)c3ccc(N4CCC(CN5CCN(c6cc(O)c(C(=O)Nc7ccc([N+](=O)[O-])cc7Cl)cc6Cl)CC5)CC4)cc3C2=O)C(=O)N1]

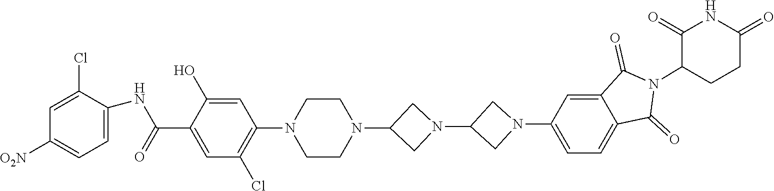 [SMILES: O=C1CCC(N2C(=O)c3ccc(N4CC(N5CC(N6CCN(c7cc(O)c(C(=O)Nc8ccc([N+](=O)[O-])cc8Cl)cc7Cl)CC6)C5)C4)cc3C2=O)C(=O)N1]

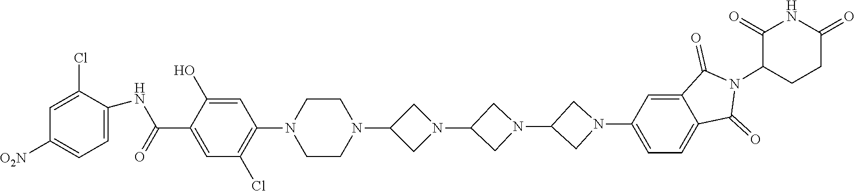 [SMILES: O=C1CCC(N2C(=O)c3ccc(N4CC(N5CC(N6CC(N7CCN(c8cc(O)c(C(=O)Nc9ccc([N+](=O)[O-])cc9Cl)cc8Cl)CC7)C6)C5)C4)cc3C2=O)C(=O)N1]

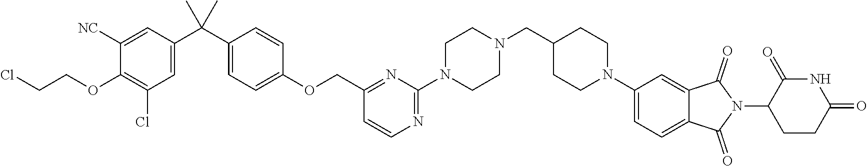 [SMILES: CC(C)(c1ccc(OCc2ccnc(N3CCN(CC4CCN(c5ccc6c(c5)C(=O)N(C5CCC(=O)NC5=O)C6=O)CC4)CC3)n2)cc1)c1cc(Cl)c(OCCCl)c(C#N)c1]

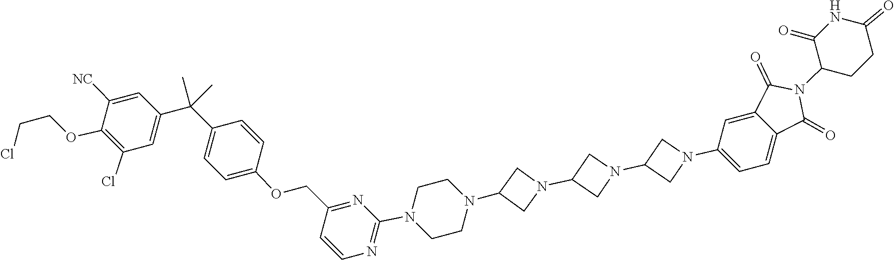 [SMILES: CC(C)(c1ccc(OCc2ccnc(N3CCN(C4CN(C5CN(C6CN(c7ccc8c(c7)C(=O)N(C7CCC(=O)NC7=O)C8=O)C6)C5)C4)CC3)n2)cc1)c1cc(Cl)c(OCCCl)c(C#N)c1]

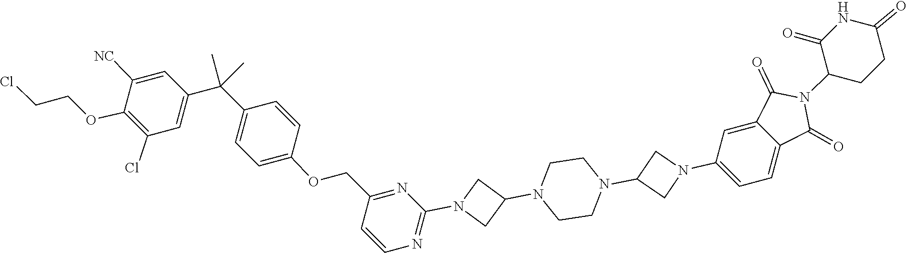 [SMILES: CC(C)(c1ccc(OCc2ccnc(N3CC(N4CCN(C5CN(c6ccc7c(c6)C(=O)N(C6CCC(=O)NC6=O)C7=O)C5)CC4)C3)n2)cc1)c1cc(Cl)c(OCCCl)c(C#N)c1]

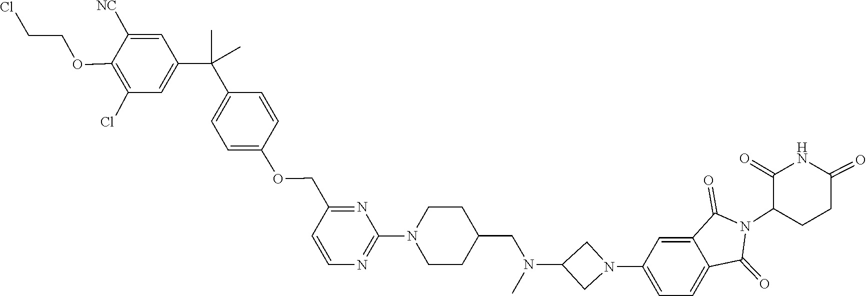 [SMILES: CN(CC1CCN(c2nccc(COc3ccc(C(C)(C)c4cc(Cl)c(OCCCl)c(C#N)c4)cc3)n2)CC1)C1CN(c2ccc3c(c2)C(=O)N(C2CCC(=O)NC2=O)C3=O)C1]

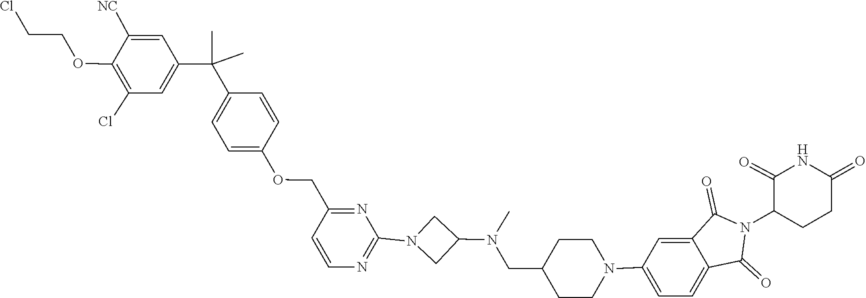 [SMILES: CN(CC1CCN(c2ccc3c(c2)C(=O)N(C2CCC(=O)NC2=O)C3=O)CC1)C1CN(c2nccc(COc3ccc(C(C)(C)c4cc(Cl)c(OCCCl)c(C#N)c4)cc3)n2)C1]

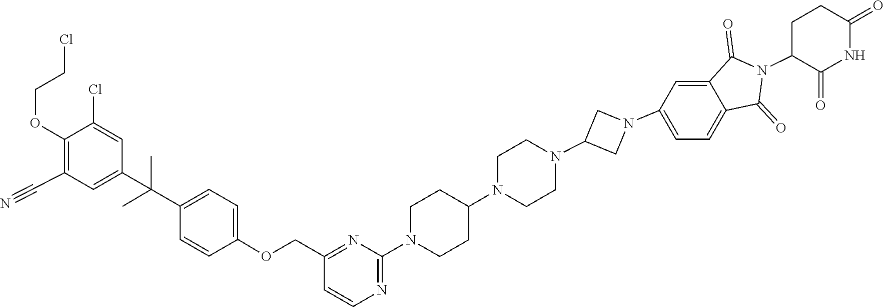 [SMILES: CC(C)(c1ccc(OCc2ccnc(N3CCC(N4CCN(C5CN(c6ccc7c(c6)C(=O)N(C6CCC(=O)NC6=O)C7=O)C5)CC4)CC3)n2)cc1)c1cc(Cl)c(OCCCl)c(C#N)c1]

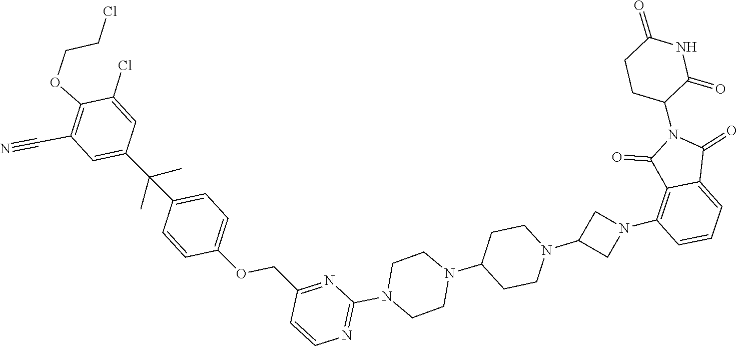 [SMILES: CC(C)(c1ccc(OCc2ccnc(N3CCN(C4CCN(C5CN(c6cccc7c6C(=O)N(C6CCC(=O)NC6=O)C7=O)C5)CC4)CC3)n2)cc1)c1cc(Cl)c(OCCCl)c(C#N)c1]

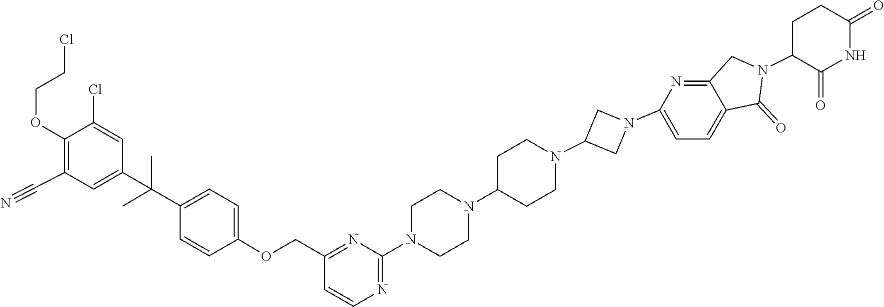 [SMILES: CC(C)(c1ccc(OCc2ccnc(N3CCN(C4CCN(C5CN(c6ccc7c(n6)CN(C6CCC(=O)NC6=O)C7=O)C5)CC4)CC3)n2)cc1)c1cc(Cl)c(OCCCl)c(C#N)c1]

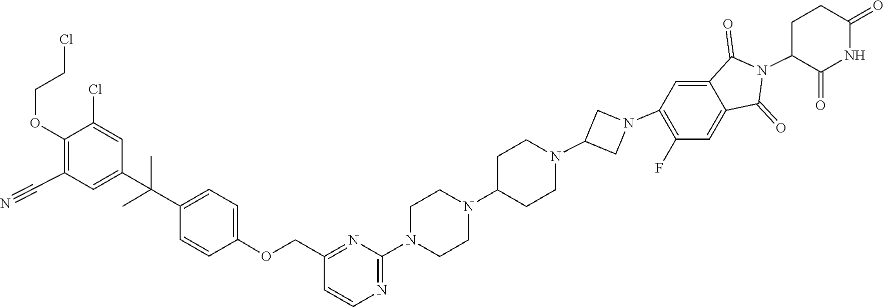 [SMILES: CC(C)(c1ccc(OCc2ccnc(N3CCN(C4CCN(C5CN(c6cc7c(cc6F)C(=O)N(C6CCC(=O)NC6=O)C7=O)C5)CC4)CC3)n2)cc1)c1cc(Cl)c(OCCCl)c(C#N)c1]

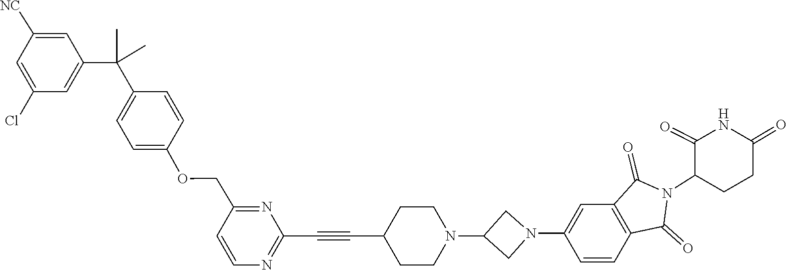 [SMILES: CC(C)(c1ccc(OCc2ccnc(C#CC3CCN(C4CN(c5ccc6c(c5)C(=O)N(C5CCC(=O)NC5=O)C6=O)C4)CC3)n2)cc1)c1cc(Cl)cc(C#N)c1]

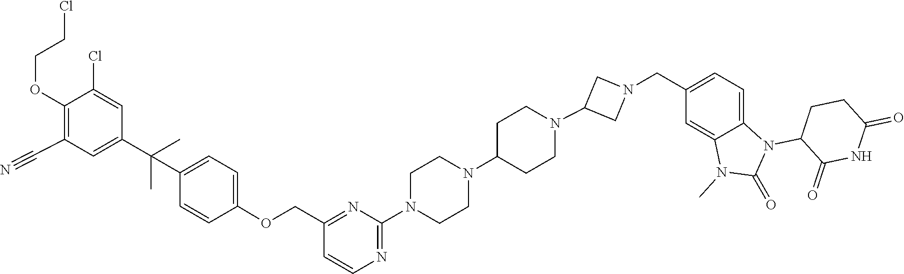 [SMILES: Cn1c(=O)n(C2CCC(=O)NC2=O)c2ccc(CN3CC(N4CCC(N5CCN(c6nccc(COc7ccc(C(C)(C)c8cc(Cl)c(OCCCl)c(C#N)c8)cc7)n6)CC5)CC4)C3)cc21]

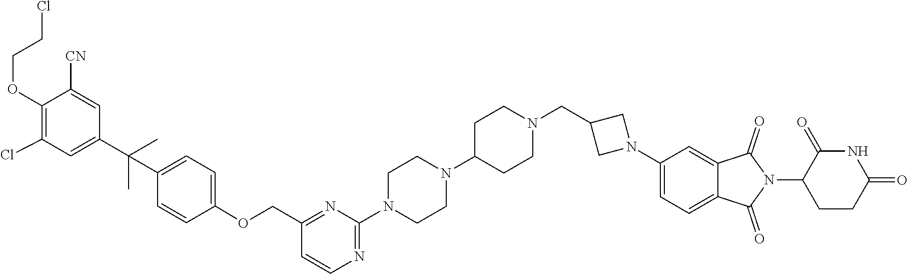 [SMILES: CC(C)(c1ccc(OCc2ccnc(N3CCN(C4CCN(CC5CN(c6ccc7c(c6)C(=O)N(C6CCC(=O)NC6=O)C7=O)C5)CC4)CC3)n2)cc1)c1cc(Cl)c(OCCCl)c(C#N)c1]

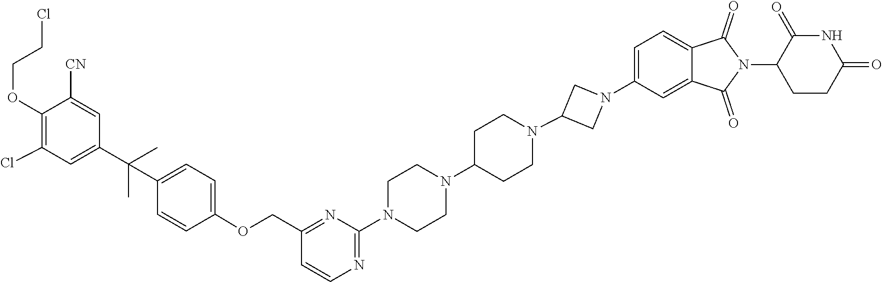 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)c(OCCCl)c(C#N)c1]

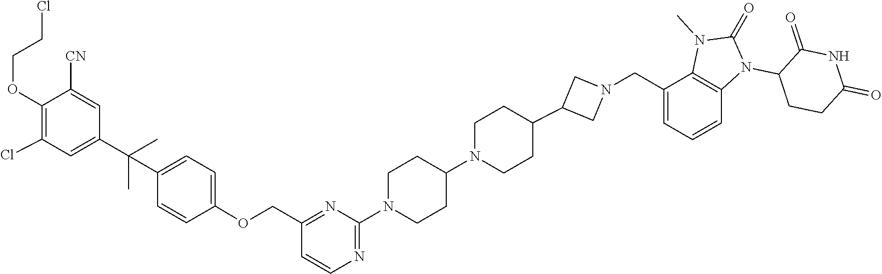 [SMILES: Cn1c(=O)n(C2CCC(=O)NC2=O)c2cccc(CN3CC(C4CCN(C5CCN(c6nccc(COc7ccc(C(C)(C)c8cc(Cl)c(OCCCl)c(C#N)c8)cc7)n6)CC5)CC4)C3)c21]

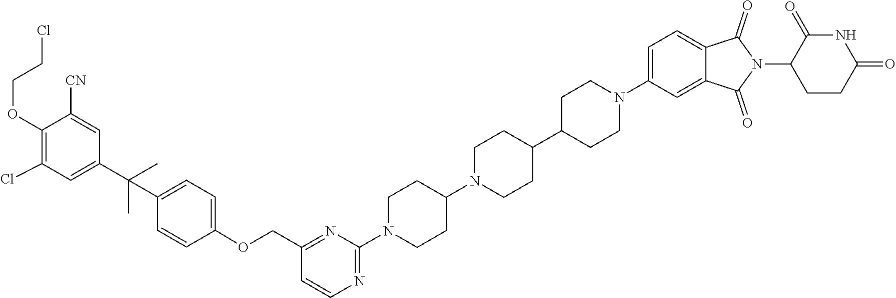 [SMILES: CC(C)(c1ccc(OCc2ccnc(N3CCC(N4CCC(C5CCN(c6ccc7c(c6)C(=O)N(C6CCC(=O)NC6=O)C7=O)CC5)CC4)CC3)n2)cc1)c1cc(Cl)c(OCCCl)c(C#N)c1]

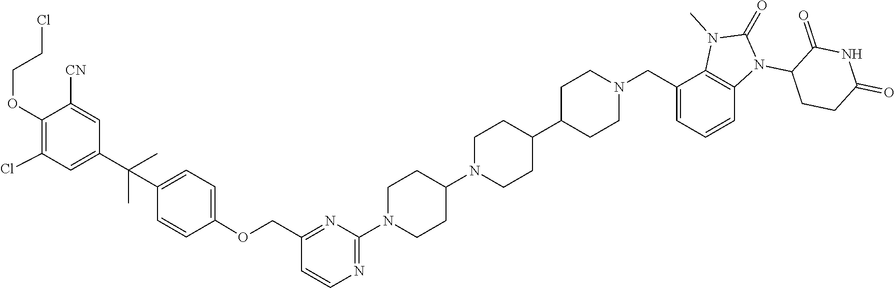 [SMILES: Cn1c(=O)n(C2CCC(=O)NC2=O)c2cccc(CN3CCC(C4CCN(C5CCN(c6nccc(COc7ccc(C(C)(C)c8cc(Cl)c(OCCCl)c(C#N)c8)cc7)n6)CC5)CC4)CC3)c21]

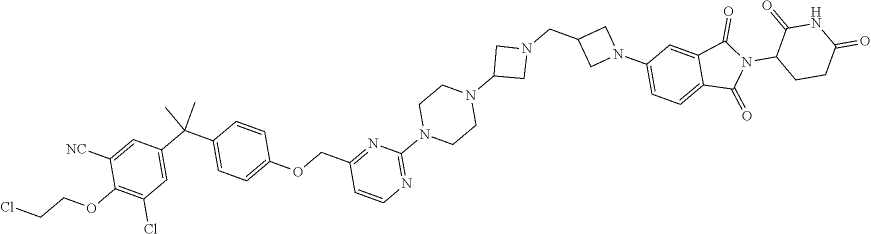 [SMILES: CC(C)(c1ccc(OCc2ccnc(N3CCN(C4CN(CC5CN(c6ccc7c(c6)C(=O)N(C6CCC(=O)NC6=O)C7=O)C5)C4)CC3)n2)cc1)c1cc(Cl)c(OCCCl)c(C#N)c1]

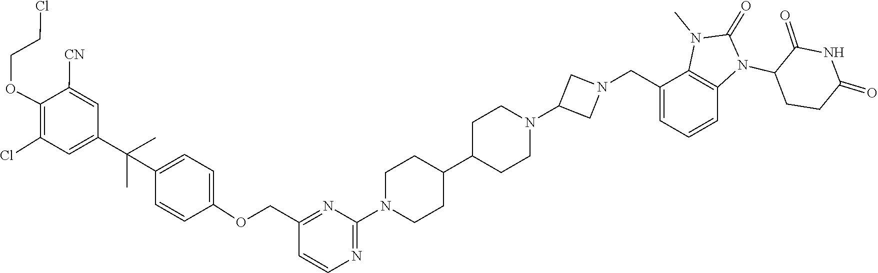 [SMILES: Cn1c(=O)n(C2CCC(=O)NC2=O)c2cccc(CN3CC(N4CCC(C5CCN(c6nccc(COc7ccc(C(C)(C)c8cc(Cl)c(OCCCl)c(C#N)c8)cc7)n6)CC5)CC4)C3)c21]

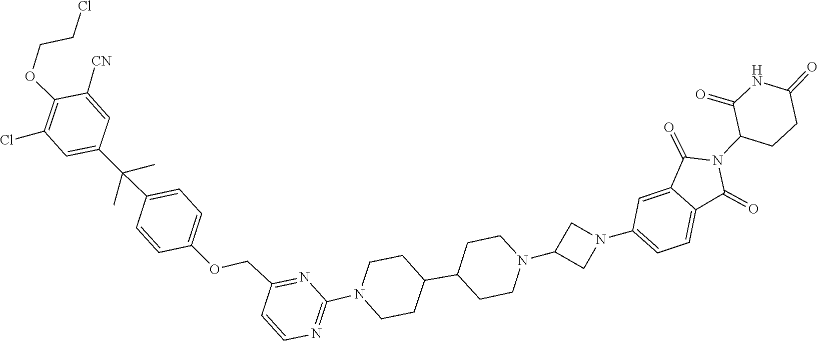 [SMILES: CC(C)(c1ccc(OCc2ccnc(N3CCC(C4CCN(C5CN(c6ccc7c(c6)C(=O)N(C6CCC(=O)NC6=O)C7=O)C5)CC4)CC3)n2)cc1)c1cc(Cl)c(OCCCl)c(C#N)c1]

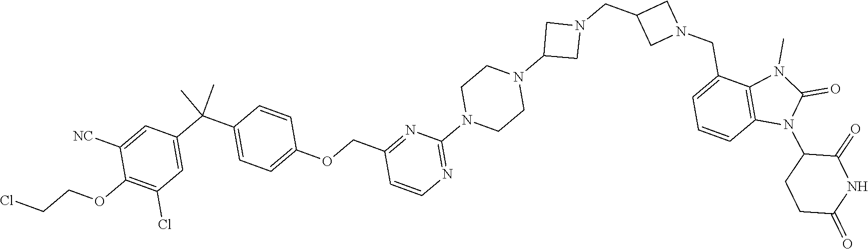 [SMILES: Cn1c(=O)n(C2CCC(=O)NC2=O)c2cccc(CN3CC(CN4CC(N5CCN(c6nccc(COc7ccc(C(C)(C)c8cc(Cl)c(OCCCl)c(C#N)c8)cc7)n6)CC5)C4)C3)c21]

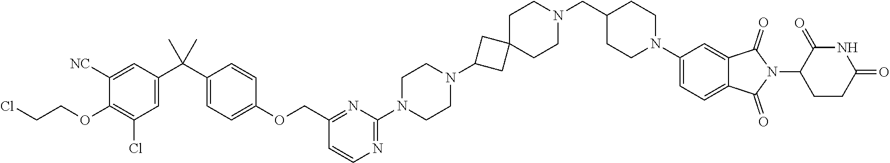 [SMILES: CC(C)(c1ccc(OCc2ccnc(N3CCN(C4CC5(CCN(CC6CCN(c7ccc8c(c7)C(=O)N(C7CCC(=O)NC7=O)C8=O)CC6)CC5)C4)CC3)n2)cc1)c1cc(Cl)c(OCCCl)c(C#N)c1]

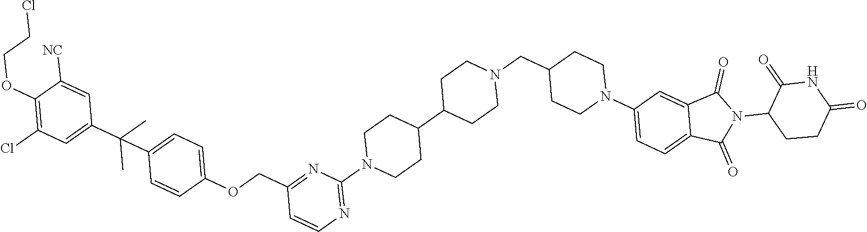 [SMILES: CC(C)(c1ccc(OCc2ccnc(N3CCC(C4CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

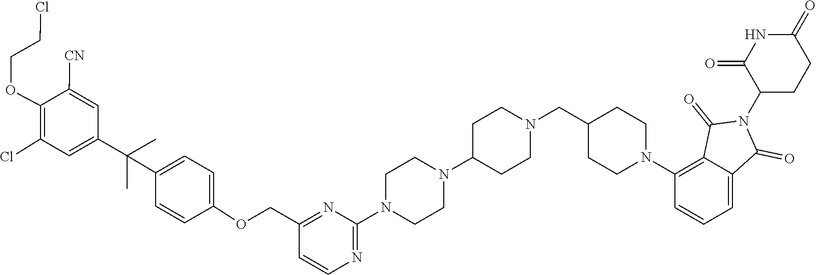 [SMILES: CC(C)(c1ccc(OCc2ccnc(N3CCN(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

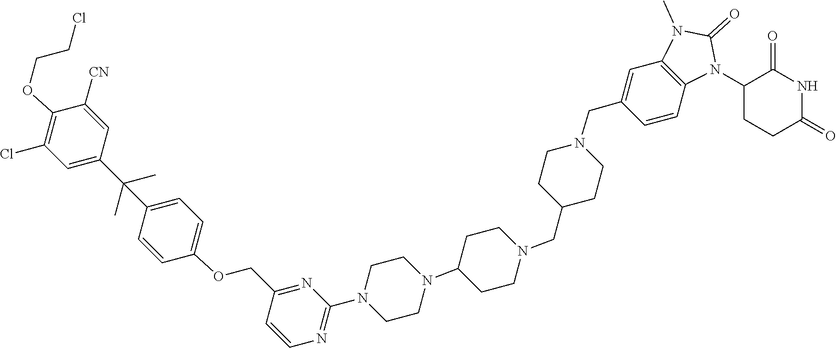 [SMILES: Cn1c(=O)n(C2CCC(=O)NC2=O)c2ccc(CN3CCC(CN4CCC(N5CCN(c6nccc(COc7ccc(C(C)(C)c8cc(Cl)c(OCCCl)c(C#N)c8)cc7)n6)CC5)CC4)CC3)cc21]

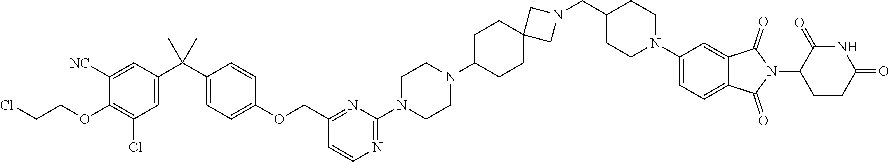 [SMILES: CC(C)(c1ccc(OCc2ccnc(N3CCN(C4CCC5(CC4)CN(CC4CCN(c6ccc7c(c6)C(=O)N(C6CCC(=O)NC6=O)C7=O)CC4)C5)CC3)n2)cc1)c1cc(Cl)c(OCCCl)c(C#N)c1]

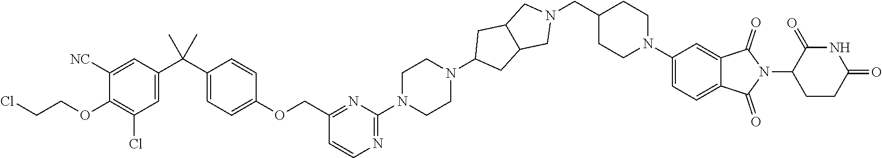 [SMILES: CC(C)(c1ccc(OCc2ccnc(N3CCN(C4CC5CN(CC6CCN(c7ccc8c(c7)C(=O)N(C7CCC(=O)NC7=O)C8=O)CC6)CC5C4)CC3)n2)cc1)c1cc(Cl)c(OCCCl)c(C#N)c1]

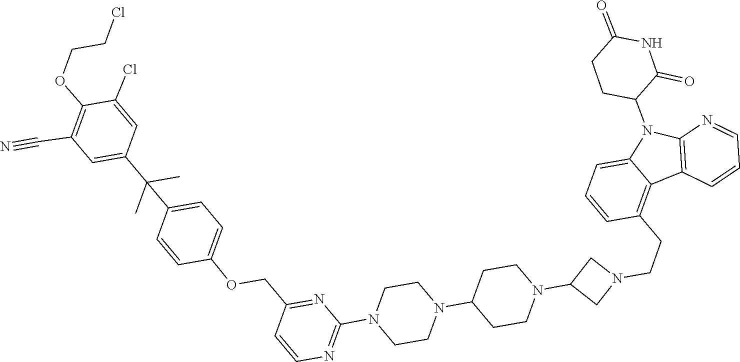 [SMILES: CC(C)(c1ccc(OCc2ccnc(N3CCN(C4CCN(C5CN(CCc6cccc7c6c6cccnc6n7C6CCC(=O)NC6=O)C5)CC4)CC3)n2)cc1)c1cc(Cl)c(OCCCl)c(C#N)c1]

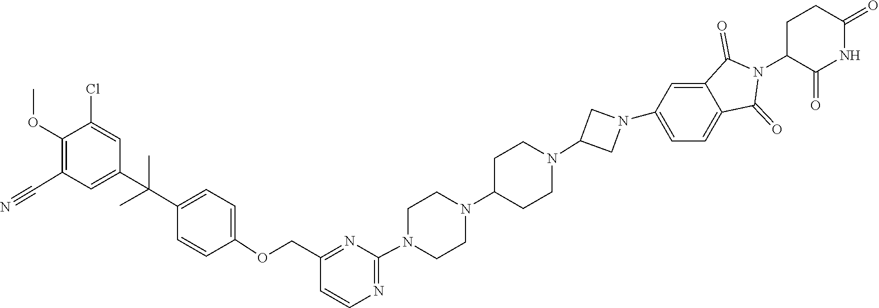 [SMILES: COc1c(Cl)cc(C(C)(C)c2ccc(OCc3ccnc(N4CCN(C5CCN(C6CN(c7ccc8c(c7)C(=O)N(C7CCC(=O)NC7=O)C8=O)C6)CC5)CC4)n3)cc2)cc1C#N]

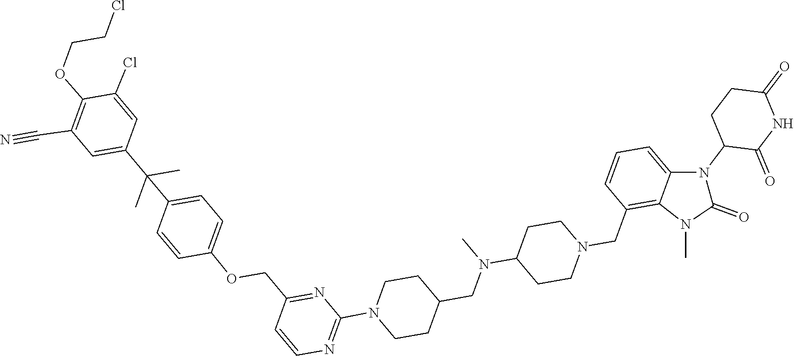 [SMILES: CN(CC1CCN(c2nccc(COc3ccc(C(C)(C)c4cc(Cl)c(OCCCl)c(C#N)c4)cc3)n2)CC1)C1CCN(Cc2cccc3c2n(C)c(=O)n3C2CCC(=O)NC2=O)CC1]

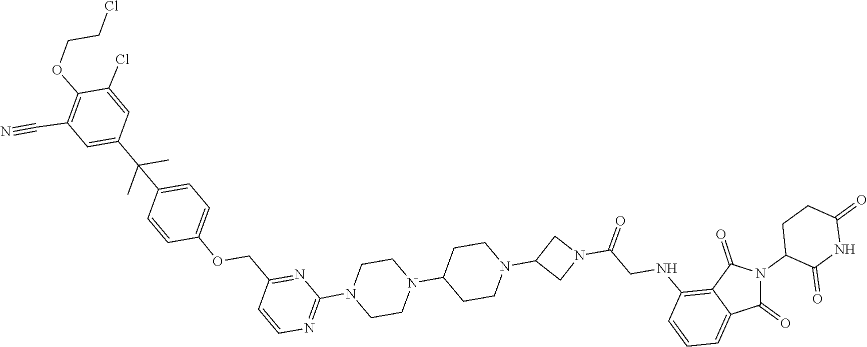 [SMILES: CC(C)(c1ccc(OCc2ccnc(N3CCN(C4CCN(C5CN(C(=O)CNc6cccc7c6C(=O)N(C6CCC(=O)NC6=O)C7=O)C5)CC4)CC3)n2)cc1)c1cc(Cl)c(OCCCl)c(C#N)c1]

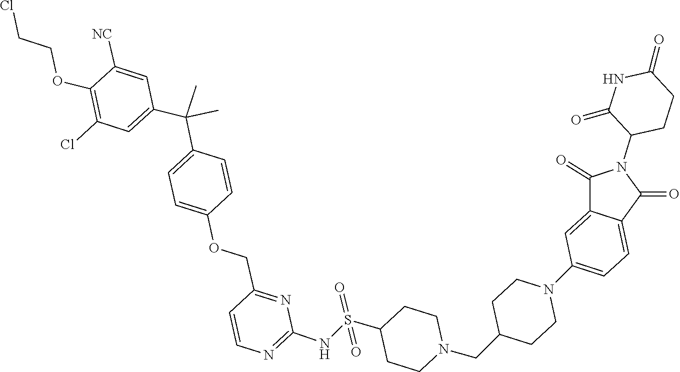 [SMILES: CC(C)(c1ccc(OCc2ccnc(NS(=O)(=O)C3CCN(CC4CCN(c5ccc6c(c5)C(=O)N(C5CCC(=O)NC5=O)C6=O)CC4)CC3)n2)cc1)c1cc(Cl)c(OCCCl)c(C#N)c1]

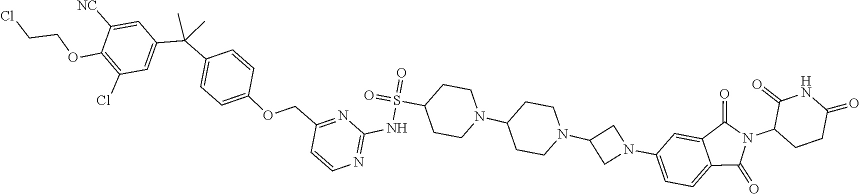 [SMILES: CC(C)(c1ccc(OCc2ccnc(NS(=O)(=O)C3CCN(C4CCN(C5CN(c6ccc7c(c6)C(=O)N(C6CCC(=O)NC6=O)C7=O)C5)CC4)CC3)n2)cc1)c1cc(Cl)c(OCCCl)c(C#N)c1]

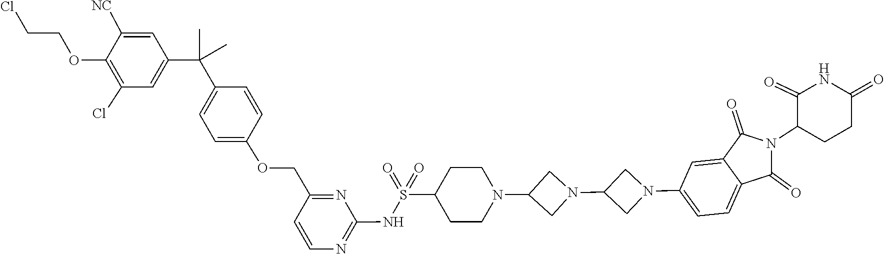 [SMILES: CC(C)(c1ccc(OCc2ccnc(NS(=O)(=O)C3CCN(C4CN(C5CN(c6ccc7c(c6)C(=O)N(C6CCC(=O)NC6=O)C7=O)C5)C4)CC3)n2)cc1)c1cc(Cl)c(OCCCl)c(C#N)c1]

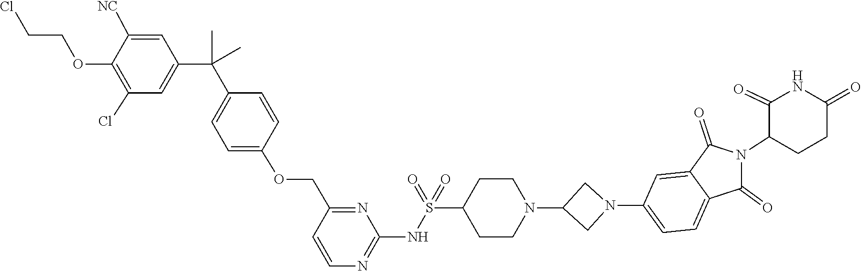 [SMILES: CC(C)(c1ccc(OCc2ccnc(NS(=O)(=O)C3CCN(C4CN(c5ccc6c(c5)C(=O)N(C5CCC(=O)NC5=O)C6=O)C4)CC3)n2)cc1)c1cc(Cl)c(OCCCl)c(C#N)c1]

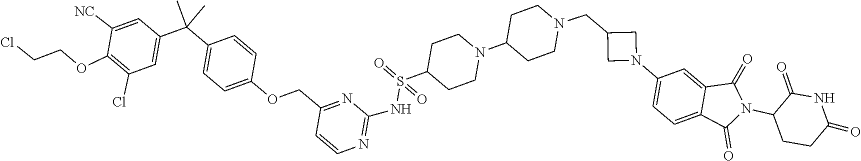 [SMILES: CC(C)(c1ccc(OCc2ccnc(NS(=O)(=O)C3CCN(C4CCN(CC5CN(c6ccc7c(c6)C(=O)N(C6CCC(=O)NC6=O)C7=O)C5)CC4)CC3)n2)cc1)c1cc(Cl)c(OCCCl)c(C#N)c1]

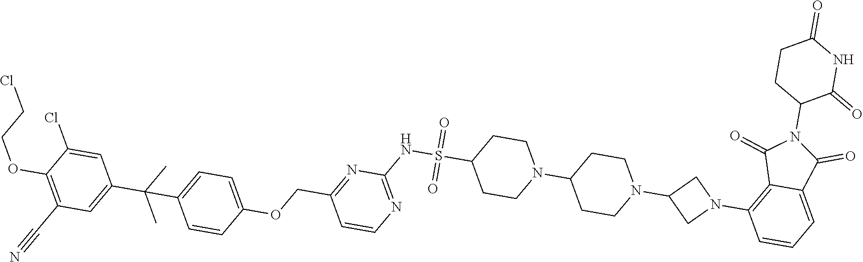 [SMILES: CC(C)(c1ccc(OCc2ccnc(NS(=O)(=O)C3CCN(C4CCN(C5CN(c6cccc7c6C(=O)N(C6CCC(=O)NC6=O)C7=O)C5)CC4)CC3)n2)cc1)c1cc(Cl)c(OCCCl)c(C#N)c1]

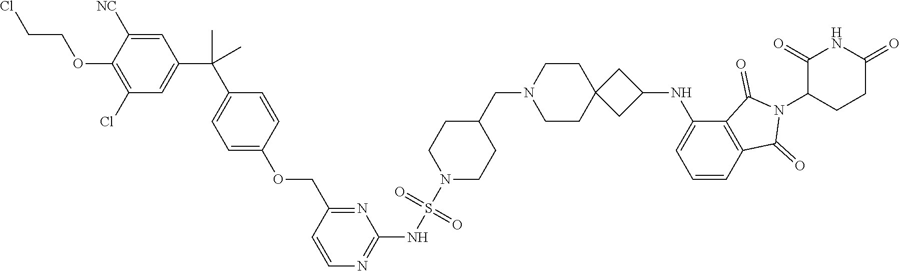 [SMILES: CC(C)(c1ccc(OCc2ccnc(NS(=O)(=O)N3CCC(CN4CCC5(CC4)CC(Nc4cccc6c4C(=O)N(C4CCC(=O)NC4=O)C6=O)C5)CC3)n2)cc1)c1cc(Cl)c(OCCCl)c(C#N)c1]

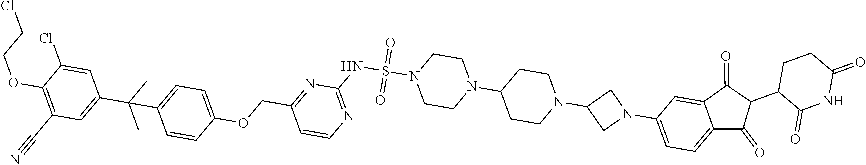 [SMILES: CC(C)(c1ccc(OCc2ccnc(NS(=O)(=O)N3CCN(C4CCN(C5CN(c6ccc7c(c6)C(=O)C(C6CCC(=O)NC6=O)C7=O)C5)CC4)CC3)n2)cc1)c1cc(Cl)c(OCCCl)c(C#N)c1]

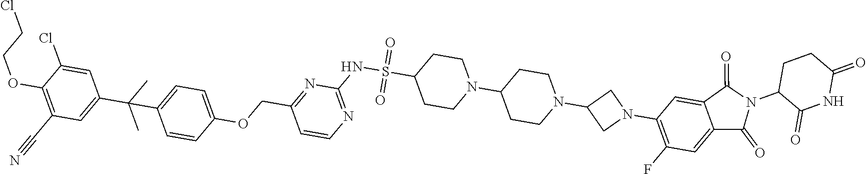 [SMILES: CC(C)(c1ccc(OCc2ccnc(NS(=O)(=O)C3CCN(C4CCN(C5CN(c6cc7c(cc6F)C(=O)N(C6CCC(=O)NC6=O)C7=O)C5)CC4)CC3)n2)cc1)c1cc(Cl)c(OCCCl)c(C#N)c1]

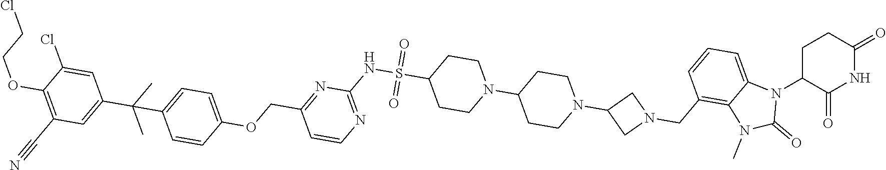 [SMILES: Cn1c(=O)n(C2CCC(=O)NC2=O)c2cccc(CN3CC(N4CCC(N5CCC(S(=O)(=O)Nc6nccc(COc7ccc(C(C)(C)c8cc(Cl)c(OCCCl)c(C#N)c8)cc7)n6)CC5)CC4)C3)c21]

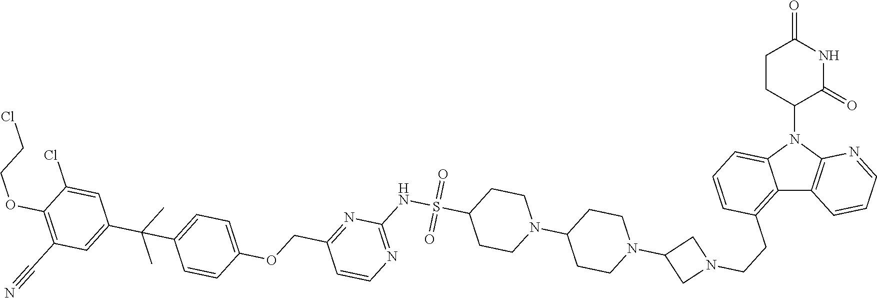 [SMILES: CC(C)(c1ccc(OCc2ccnc(NS(=O)(=O)C3CCN(C4CCN(C5CN(CCc6cccc7c6c6cccnc6n7C6CCC(=O)NC6=O)C5)CC4)CC3)n2)cc1)c1cc(Cl)c(OCCCl)c(C#N)c1]

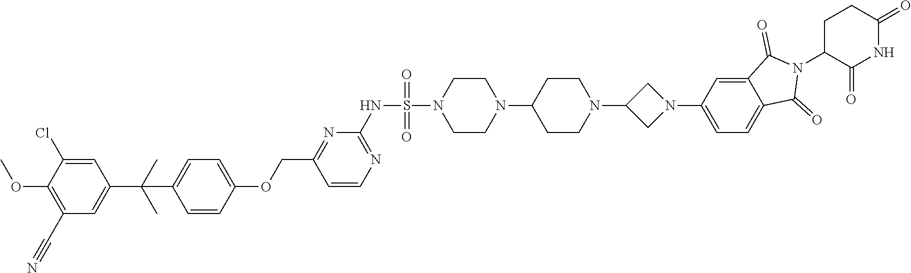 [SMILES: COc1c(Cl)cc(C(C)(C)c2ccc(OCc3ccnc(NS(=O)(=O)N4CCN(C5CCN(C6CN(c7ccc8c(c7)C(=O)N(C7CCC(=O)NC7=O)C8=O)C6)CC5)CC4)n3)cc2)cc1C#N]

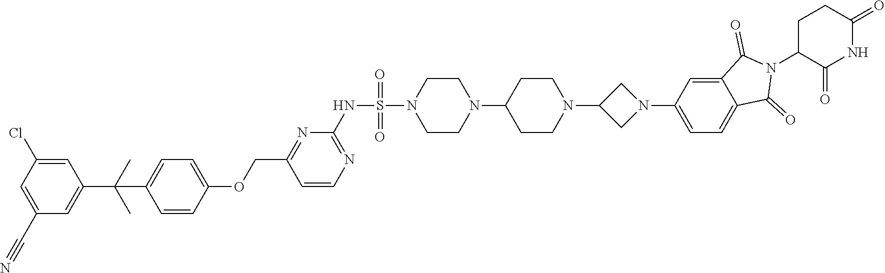 [SMILES: CC(C)(c1ccc(OCc2ccnc(NS(=O)(=O)N3CCN(C4CCN(C5CN(c6ccc7c(c6)C(=O)N(C6CCC(=O)NC6=O)C7=O)C5)CC4)CC3)n2)cc1)c1cc(Cl)cc(C#N)c1]

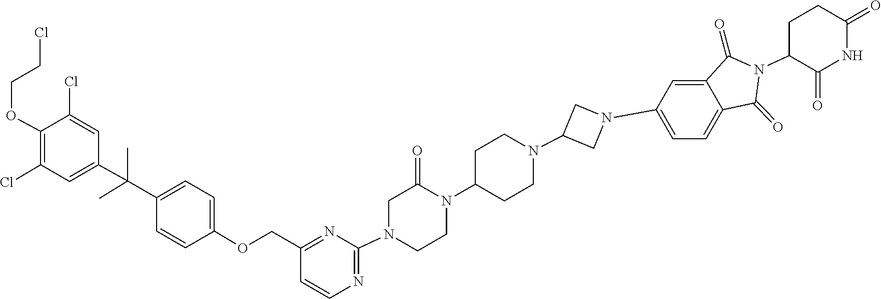 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)C(=O)C3)n2)cc1)c1cc(Cl)c(OCCCl)c(Cl)c1]

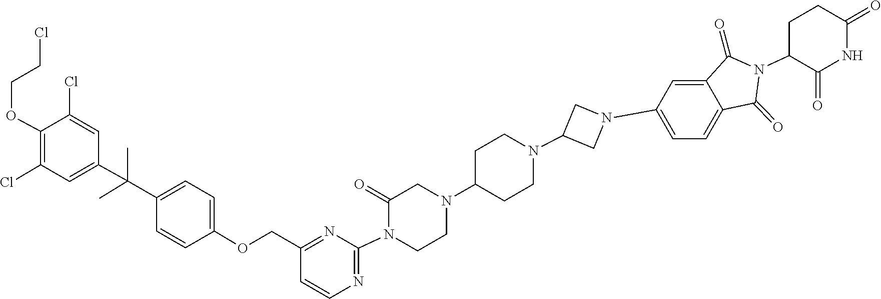 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3=O)n2)cc1)c1cc(Cl)c(OCCCl)c(Cl)c1]

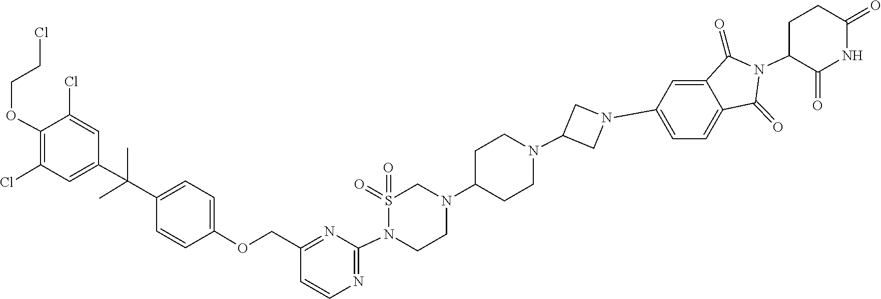 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CS3(=O)=O)n2)cc1)c1cc(Cl)c(OCCCl)c(Cl)c1]

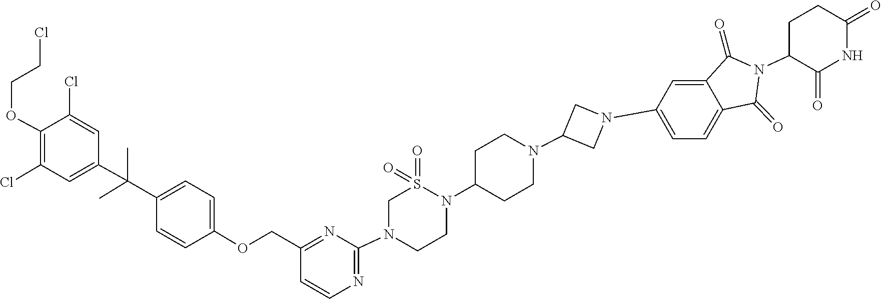 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)S(=O)(=O)C3)n2)cc1)c1cc(Cl)c(OCCCl)c(Cl)c1]

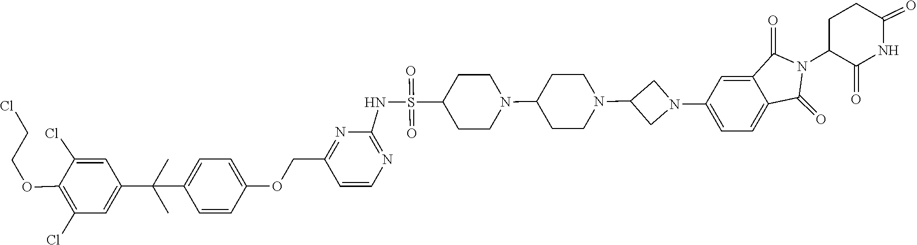 [SMILES: CC(C)(c1ccc(OCc2ccnc(NS(=O)(=O)C3CCN(C4CCN(C5CN(c6ccc7c(c6)C(=O)N(C6CCC(=O)NC6=O)C7=O)C5)CC4)CC3)n2)cc1)c1cc(Cl)c(OCCCl)c(Cl)c1]

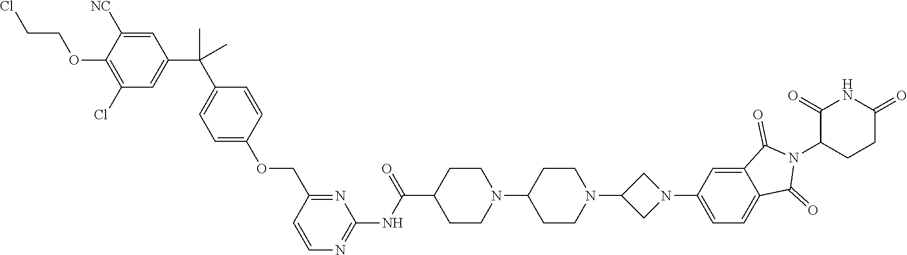 [SMILES: CC(C)(c1ccc(OCc2ccnc(NC(=O)C3CCN(C4CCN(C5CN(c6ccc7c(c6)C(=O)N(C6CCC(=O)NC6=O)C7=O)C5)CC4)CC3)n2)cc1)c1cc(Cl)c(OCCCl)c(C#N)c1]

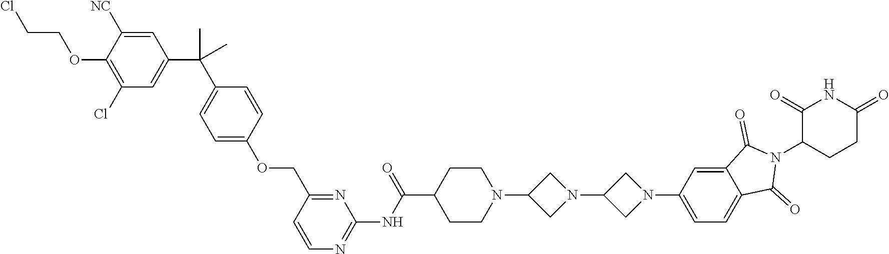 [SMILES: CC(C)(c1ccc(OCc2ccnc(NC(=O)C3CCN(C4CN(C5CN(c6ccc7c(c6)C(=O)N(C6CCC(=O)NC6=O)C7=O)C5)C4)CC3)n2)cc1)c1cc(Cl)c(OCCCl)c(C#N)c1]

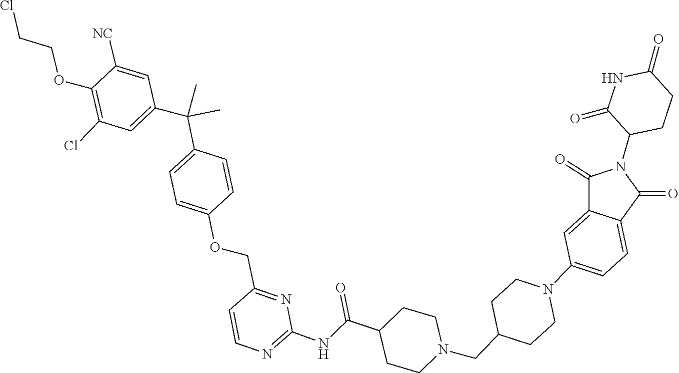 [SMILES: CC(C)(c1ccc(OCc2ccnc(NC(=O)C3CCN(CC4CCN(c5ccc6c(c5)C(=O)N(C5CCC(=O)NC5=O)C6=O)CC4)CC3)n2)cc1)c1cc(Cl)c(OCCCl)c(C#N)c1]

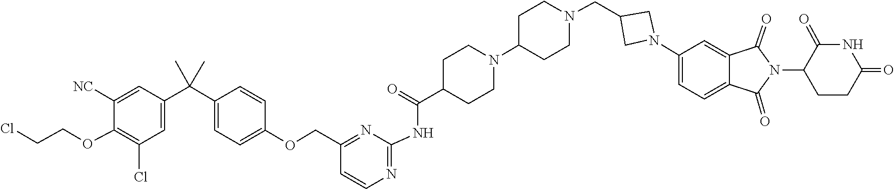 [SMILES: CC(C)(c1ccc(OCc2ccnc(NC(=O)C3CCN(C4CCN(CC5CN(c6ccc7c(c6)C(=O)N(C6CCC(=O)NC6=O)C7=O)C5)CC4)CC3)n2)cc1)c1cc(Cl)c(OCCCl)c(C#N)c1]

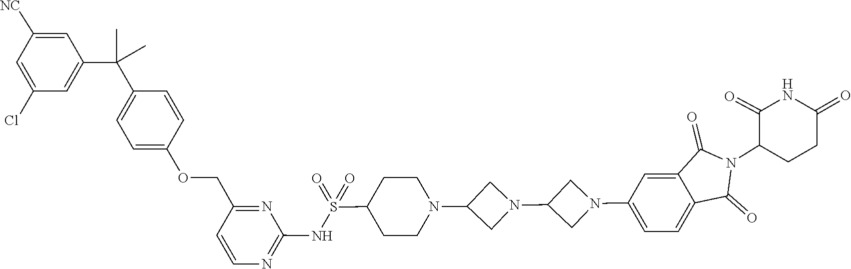 [SMILES: CC(C)(c1ccc(OCc2ccnc(NS(=O)(=O)C3CCN(C4CN(C5CN(c6ccc7c(c6)C(=O)N(C6CCC(=O)NC6=O)C7=O)C5)C4)CC3)n2)cc1)c1cc(Cl)cc(C#N)c1]

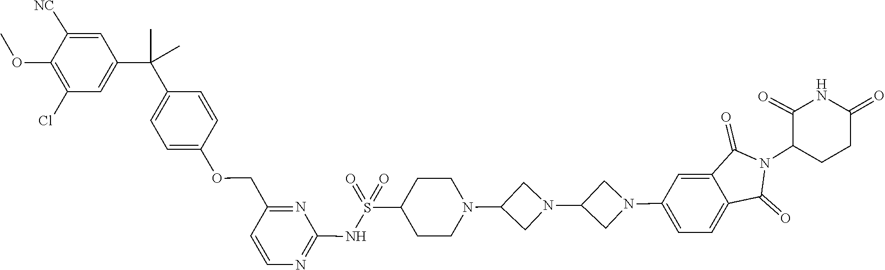 [SMILES: COc1c(Cl)cc(C(C)(C)c2ccc(OCc3ccnc(NS(=O)(=O)C4CCN(C5CN(C6CN(c7ccc8c(c7)C(=O)N(C7CCC(=O)NC7=O)C8=O)C6)C5)CC4)n3)cc2)cc1C#N]

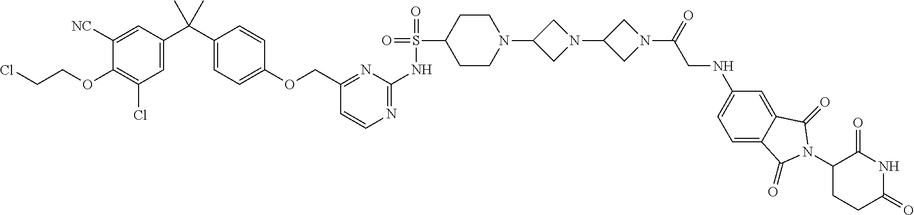 [SMILES: CC(C)(c1ccc(OCc2ccnc(NS(=O)(=O)C3CCN(C4CN(C5CN(C(=O)CNc6ccc7c(c6)C(=O)N(C6CCC(=O)NC6=O)C7=O)C5)C4)CC3)n2)cc1)c1cc(Cl)c(OCCCl)c(C#N)c1]

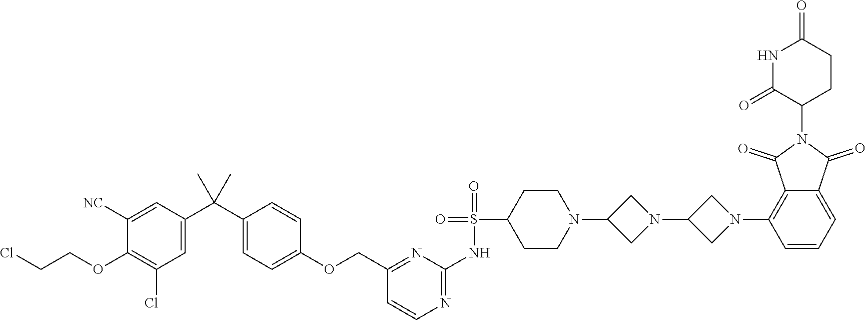 [SMILES: CC(C)(c1ccc(OCc2ccnc(NS(=O)(=O)C3CCN(C4CN(C5CN(c6cccc7c6C(=O)N(C6CCC(=O)NC6=O)C7=O)C5)C4)CC3)n2)cc1)c1cc(Cl)c(OCCCl)c(C#N)c1]

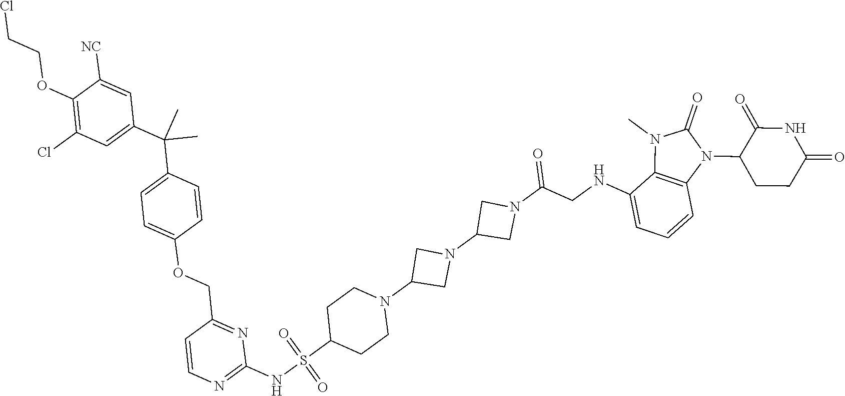 [SMILES: Cn1c(=O)n(C2CCC(=O)NC2=O)c2cccc(NCC(=O)N3CC(N4CC(N5CCC(S(=O)(=O)Nc6nccc(COc7ccc(C(C)(C)c8cc(Cl)c(OCCCl)c(C#N)c8)cc7)n6)CC5)C4)C3)c21]

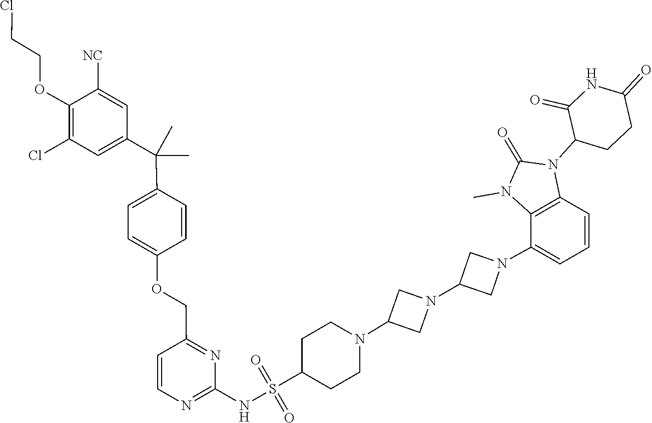 [SMILES: Cn1c(=O)n(C2CCC(=O)NC2=O)c2cccc(N3CC(N4CC(N5CCC(S(=O)(=O)Nc6nccc(COc7ccc(C(C)(C)c8cc(Cl)c(OCCCl)c(C#N)c8)cc7)n6)CC5)C4)C3)c21]

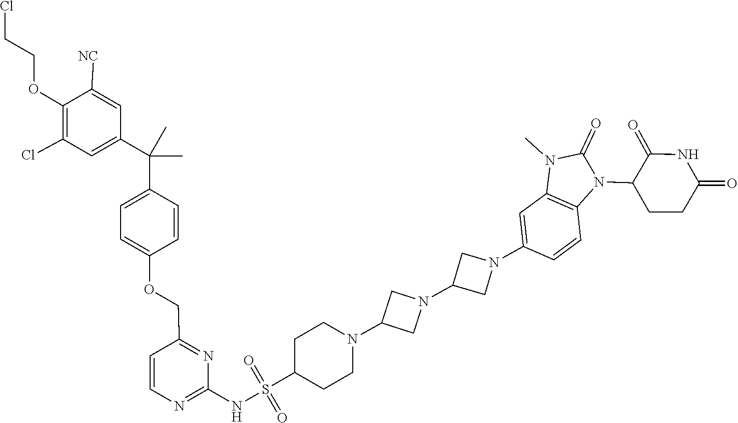 [SMILES: Cn1c(=O)n(C2CCC(=O)NC2=O)c2ccc(N3CC(N4CC(N5CCC(S(=O)(=O)Nc6nccc(COc7ccc(C(C)(C)c8cc(Cl)c(OCCCl)c(C#N)c8)cc7)n6)CC5)C4)C3)cc21]

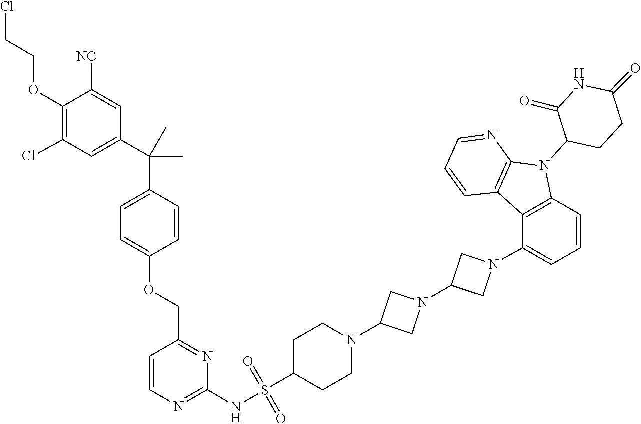 [SMILES: CC(C)(c1ccc(OCc2ccnc(NS(=O)(=O)C3CCN(C4CN(C5CN(c6cccc7c6c6cccnc6n7C6CCC(=O)NC6=O)C5)C4)CC3)n2)cc1)c1cc(Cl)c(OCCCl)c(C#N)c1]

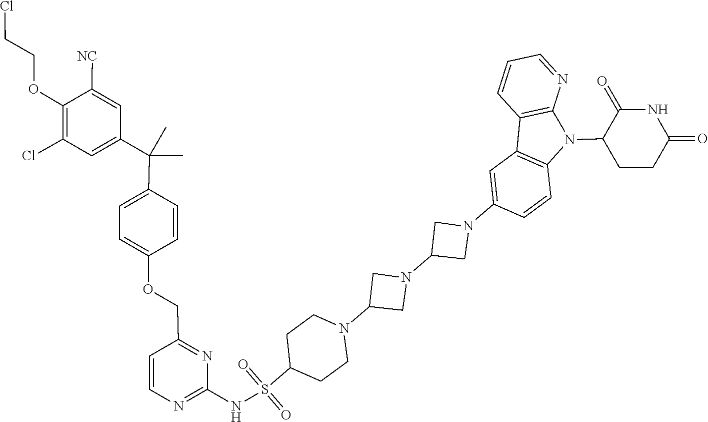 [SMILES: CC(C)(c1ccc(OCc2ccnc(NS(=O)(=O)C3CCN(C4CN(C5CN(c6ccc7c(c6)c6cccnc6n7C6CCC(=O)NC6=O)C5)C4)CC3)n2)cc1)c1cc(Cl)c(OCCCl)c(C#N)c1]

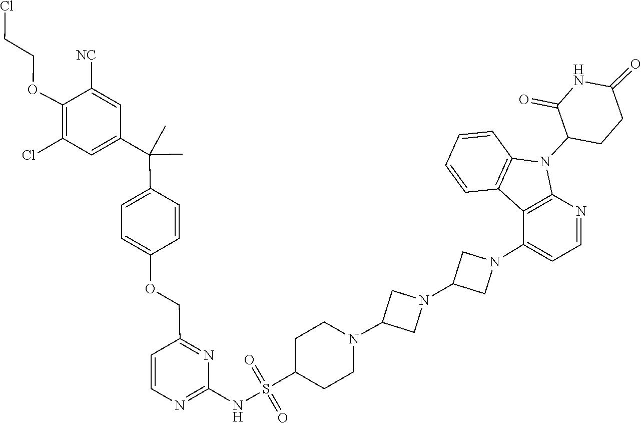 [SMILES: CC(C)(c1ccc(OCc2ccnc(NS(=O)(=O)C3CCN(C4CN(C5CN(c6ccnc7c6c6ccccc6n7C6CCC(=O)NC6=O)C5)C4)CC3)n2)cc1)c1cc(Cl)c(OCCCl)c(C#N)c1]

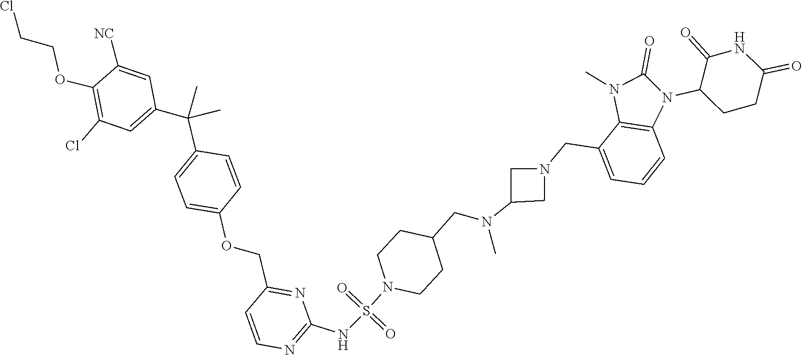 [SMILES: CN(CC1CCN(S(=O)(=O)Nc2nccc(COc3ccc(C(C)(C)c4cc(Cl)c(OCCCl)c(C#N)c4)cc3)n2)CC1)C1CN(Cc2cccc3c2n(C)c(=O)n3C2CCC(=O)NC2=O)C1]

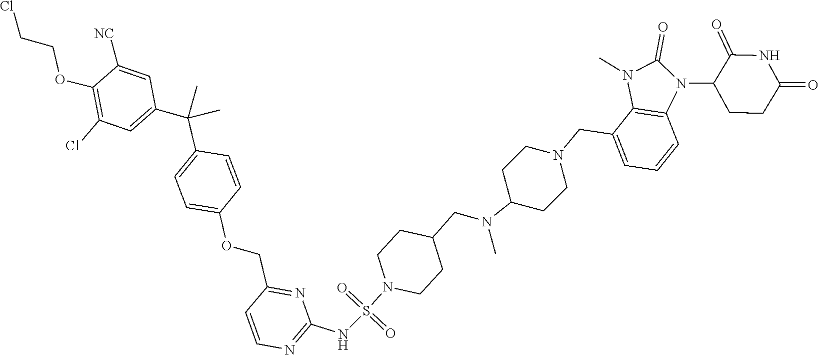 [SMILES: CN(CC1CCN(S(=O)(=O)Nc2nccc(COc3ccc(C(C)(C)c4cc(Cl)c(OCCCl)c(C#N)c4)cc3)n2)CC1)C1CCN(Cc2cccc3c2n(C)c(=O)n3C2CCC(=O)NC2=O)CC1]

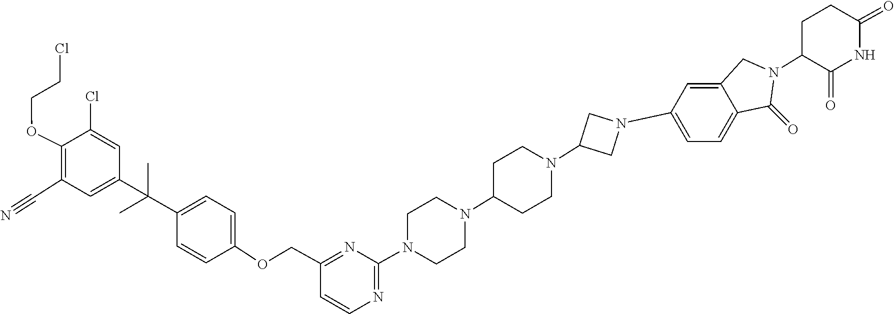 [SMILES: CC(C)(c1ccc(OCc2ccnc(N3CCN(C4CCN(C5CN(c6ccc7c(c6)CN(C6CCC(=O)NC6=O)C7=O)C5)CC4)CC3)n2)cc1)c1cc(Cl)c(OCCCl)c(C#N)c1]

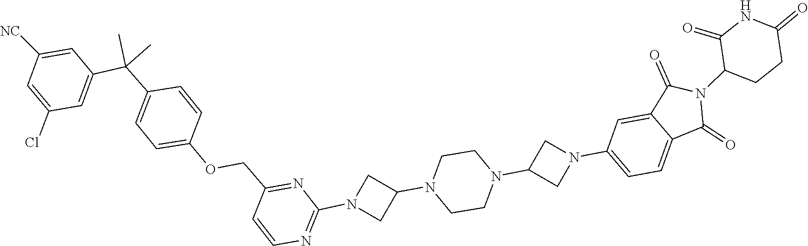 [SMILES: CC(C)(c1ccc(OCc2ccnc(N3CC(N4CCN(C5CN(c6ccc7c(c6)C(=O)N(C6CCC(=O)NC6=O)C7=O)C5)CC4)C3)n2)cc1)c1cc(Cl)cc(C#N)c1]

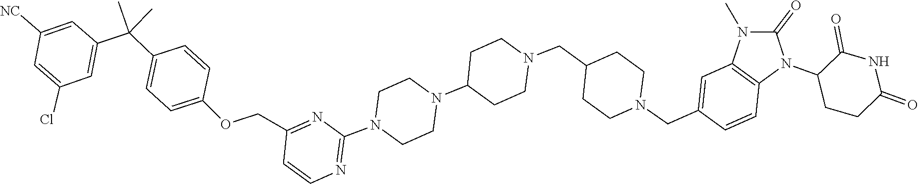 [SMILES: Cn1c(=O)n(C2CCC(=O)NC2=O)c2ccc(CN3CCC(CN4CCC(N5CCN(c6nccc(COc7ccc(C(C)(C)c8cc(Cl)cc(C#N)c8)cc7)n6)CC5)CC4)CC3)cc21]

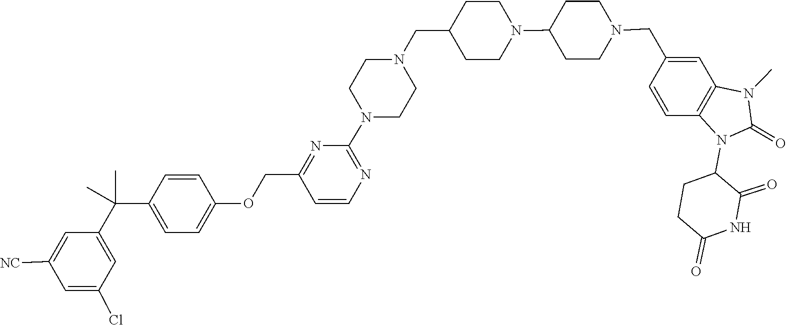 [SMILES: Cn1c(=O)n(C2CCC(=O)NC2=O)c2ccc(CN3CCC(N4CCC(CN5CCN(c6nccc(COc7ccc(C(C)(C)c8cc(Cl)cc(C#N)c8)cc7)n6)CC5)CC4)CC3)cc21]

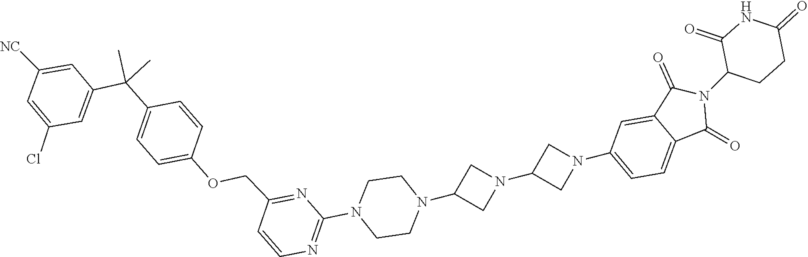 [SMILES: CC(C)(c1ccc(OCc2ccnc(N3CCN(C4CN(C5CN(c6ccc7c(c6)C(=O)N(C6CCC(=O)NC6=O)C7=O)C5)C4)CC3)n2)cc1)c1cc(Cl)cc(C#N)c1]

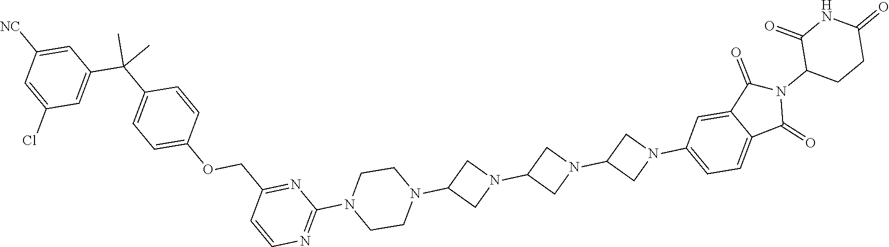 [SMILES: CC(C)(c1ccc(OCc2ccnc(N3CCN(C4CN(C5CN(C6CN(c7ccc8c(c7)C(=O)N(C7CCC(=O)NC7=O)C8=O)C6)C5)C4)CC3)n2)cc1)c1cc(Cl)cc(C#N)c1]

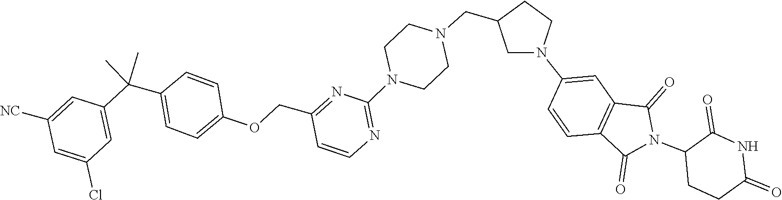 [SMILES: CC(C)(c1ccc(OCc2ccnc(N3CCN(CC4CCN(c5ccc6c(c5)C(=O)N(C5CCC(=O)NC5=O)C6=O)C4)CC3)n2)cc1)c1cc(Cl)cc(C#N)c1]

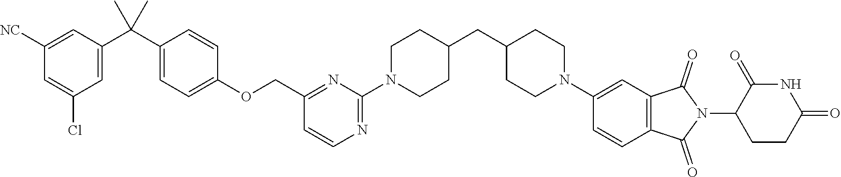 [SMILES: CC(C)(c1ccc(OCc2ccnc(N3CCC(CC4CCN(c5ccc6c(c5)C(=O)N(C5CCC(=O)NC5=O)C6=O)CC4)CC3)n2)cc1)c1cc(Cl)cc(C#N)c1]

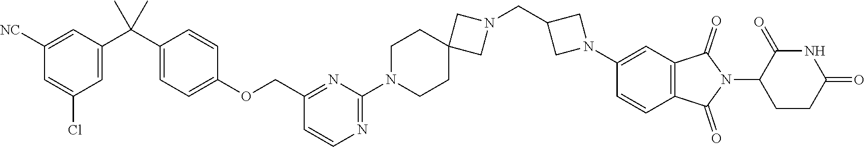 [SMILES: CC(C)(c1ccc(OCc2ccnc(N3CCC4(CC3)CN(CC3CN(c5ccc6c(c5)C(=O)N(C5CCC(=O)NC5=O)C6=O)C3)C4)n2)cc1)c1cc(Cl)cc(C#N)c1]

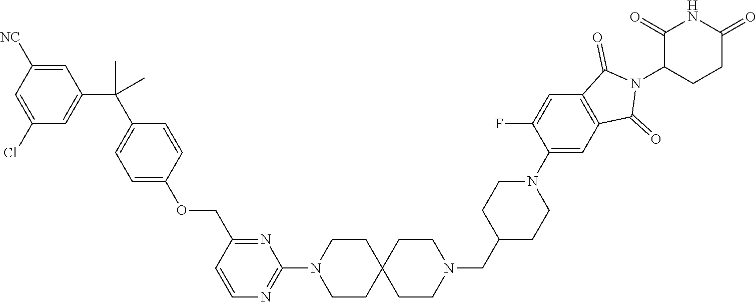 [SMILES: CC(C)(c1ccc(OCc2ccnc(N3CCC4(CCN(CC5CCN(c6cc7c(cc6F)C(=O)N(C6CCC(=O)NC6=O)C7=O)CC5)CC4)CC3)n2)cc1)c1cc(Cl)cc(C#N)c1]

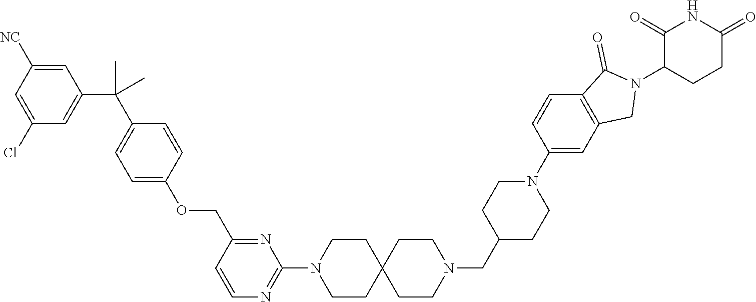 [SMILES: CC(C)(c1ccc(OCc2ccnc(N3CCC4(CCN(CC5CCN(c6ccc7c(c6)CN(C6CCC(=O)NC6=O)C7=O)CC5)CC4)CC3)n2)cc1)c1cc(Cl)cc(C#N)c1]

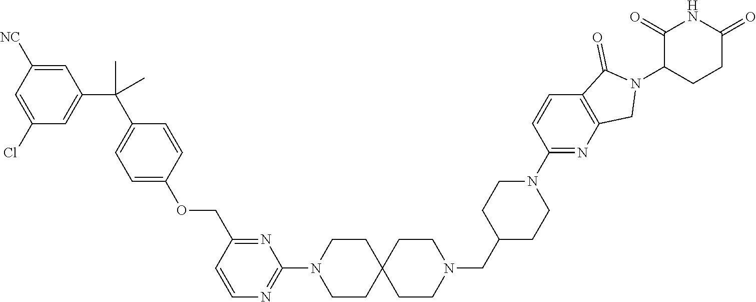 [SMILES: CC(C)(c1ccc(OCc2ccnc(N3CCC4(CCN(CC5CCN(c6ccc7c(n6)CN(C6CCC(=O)NC6=O)C7=O)CC5)CC4)CC3)n2)cc1)c1cc(Cl)cc(C#N)c1]

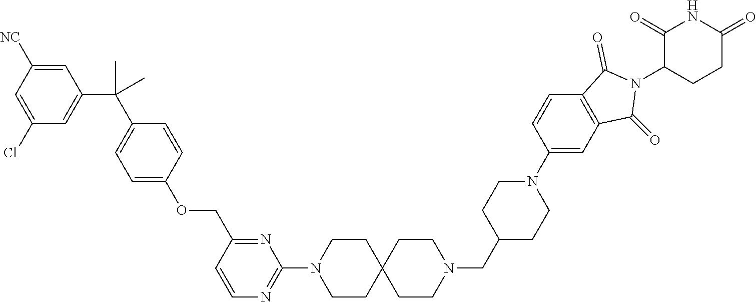 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)cc(C#N)c1]

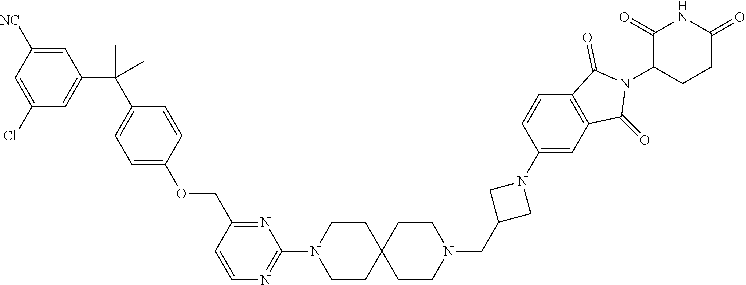 [SMILES: CC(C)(c1ccc(OCc2ccnc(N3CCC4(CCN(CC5CN(c6ccc7c(c6)C(=O)N(C6CCC(=O)NC6=O)C7=O)C5)CC4)CC3)n2)cc1)c1cc(Cl)cc(C#N)c1]

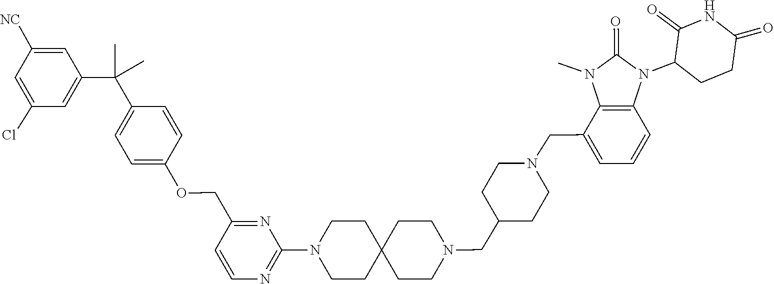 [SMILES: Cn1c(=O)n(C2CCC(=O)NC2=O)c2cccc(CN3CCC(CN4CCC5(CC4)CCN(c4nccc(COc6ccc(C(C)(C)c7cc(Cl)cc(C#N)c7)cc6)n4)CC5)CC3)c21]

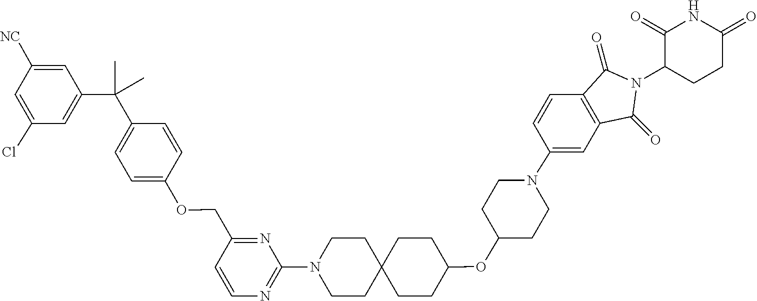 [SMILES: CC(C)(c1ccc(OCc2ccnc(N3CCC4(CCC(OC5CCN(c6ccc7c(c6)C(=O)N(C6CCC(=O)NC6=O)C7=O)CC5)CC4)CC3)n2)cc1)c1cc(Cl)cc(C#N)c1]

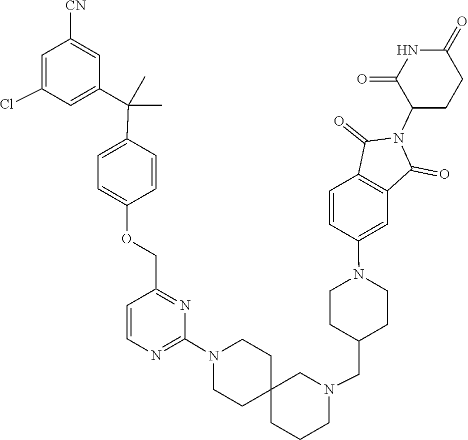 [SMILES: CC(C)(c1ccc(OCc2ccnc(N3CCC4(CCCN(CC5CCN(c6ccc7c(c6)C(=O)N(C6CCC(=O)NC6=O)C7=O)CC5)C4)CC3)n2)cc1)c1cc(Cl)cc(C#N)c1]

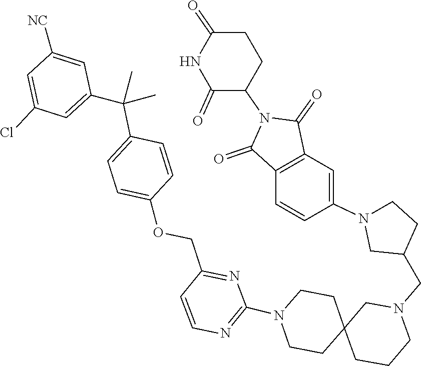 [SMILES: CC(C)(c1ccc(OCc2ccnc(N3CCC4(CCCN(CC5CCN(c6ccc7c(c6)C(=O)N(C6CCC(=O)NC6=O)C7=O)C5)C4)CC3)n2)cc1)c1cc(Cl)cc(C#N)c1]

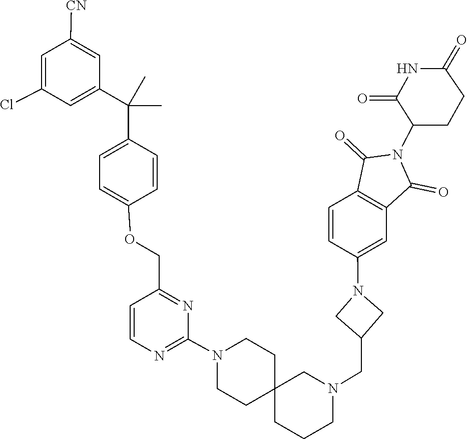 [SMILES: CC(C)(c1ccc(OCc2ccnc(N3CCC4(CCCN(CC5CN(c6ccc7c(c6)C(=O)N(C6CCC(=O)NC6=O)C7=O)C5)C4)CC3)n2)cc1)c1cc(Cl)cc(C#N)c1]

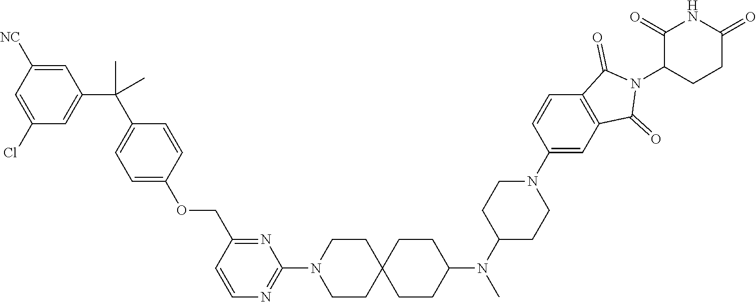 [SMILES: CN(C1CCN(c2ccc3c(c2)C(=O)N(C2CCC(=O)NC2=O)C3=O)CC1)C1CCC2(CC1)CCN(c1nccc(COc3ccc(C(C)(C)c4cc(Cl)cc(C#N)c4)cc3)n1)CC2]

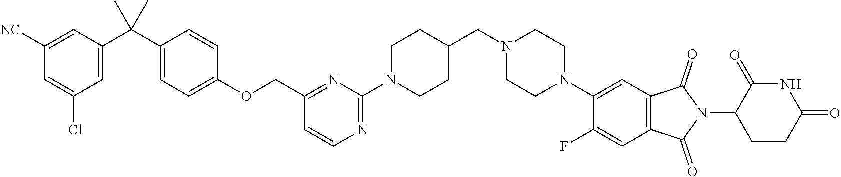 [SMILES: CC(C)(c1ccc(OCc2ccnc(N3CCC(CN4CCN(c5cc6c(cc5F)C(=O)N(C5CCC(=O)NC5=O)C6=O)CC4)CC3)n2)cc1)c1cc(Cl)cc(C#N)c1]

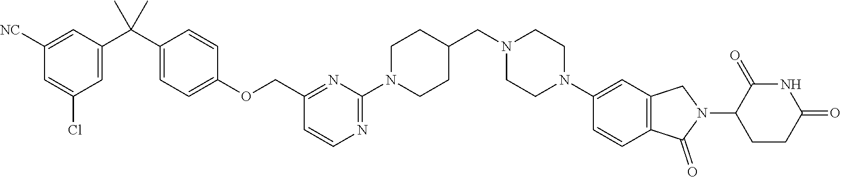 [SMILES: CC(C)(c1ccc(OCc2ccnc(N3CCC(CN4CCN(c5ccc6c(c5)CN(C5CCC(=O)NC5=O)C6=O)CC4)CC3)n2)cc1)c1cc(Cl)cc(C#N)c1]

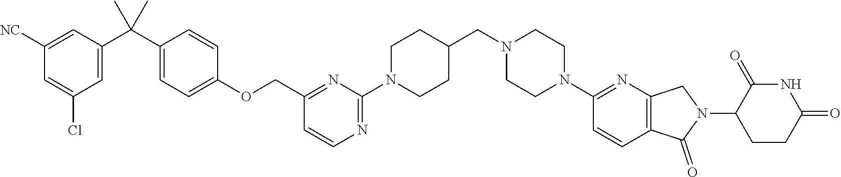 [SMILES: CC(C)(c1ccc(OCc2ccnc(N3CCC(CN4CCN(c5ccc6c(n5)CN(C5CCC(=O)NC5=O)C6=O)CC4)CC3)n2)cc1)c1cc(Cl)cc(C#N)c1]

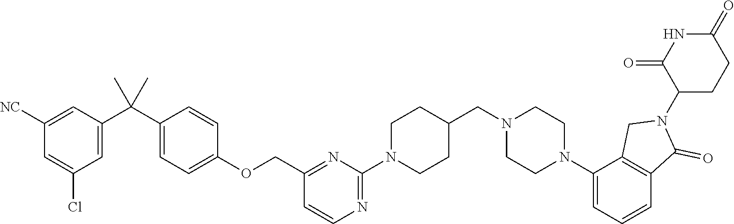 [SMILES: CC(C)(c1ccc(OCc2ccnc(N3CCC(CN4CCN(c5cccc6c5CN(C5CCC(=O)NC5=O)C6=O)CC4)CC3)n2)cc1)c1cc(Cl)cc(C#N)c1]

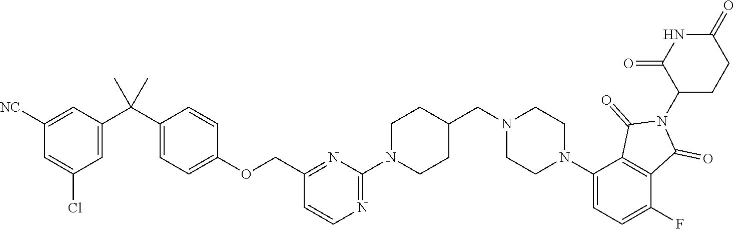 [SMILES: CC(C)(c1ccc(OCc2ccnc(N3CCC(CN4CCN(c5ccc(F)c6c5C(=O)N(C5CCC(=O)NC5=O)C6=O)CC4)CC3)n2)cc1)c1cc(Cl)cc(C#N)c1]

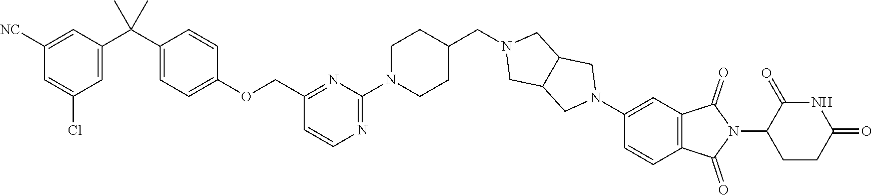 [SMILES: CC(C)(c1ccc(OCc2ccnc(N3CCC(CN4CC5CN(c6ccc7c(c6)C(=O)N(C6CCC(=O)NC6=O)C7=O)CC5C4)CC3)n2)cc1)c1cc(Cl)cc(C#N)c1]

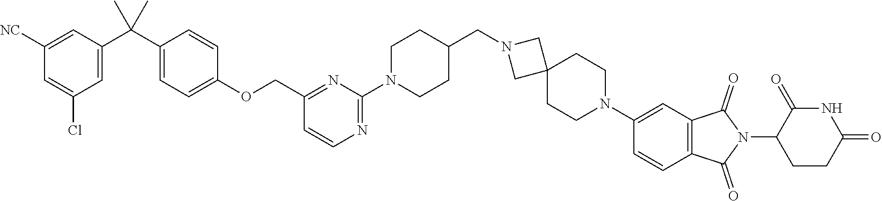 [SMILES: CC(C)(c1ccc(OCc2ccnc(N3CCC(CN4CC5(CCN(c6ccc7c(c6)C(=O)N(C6CCC(=O)NC6=O)C7=O)CC5)C4)CC3)n2)cc1)c1cc(Cl)cc(C#N)c1]

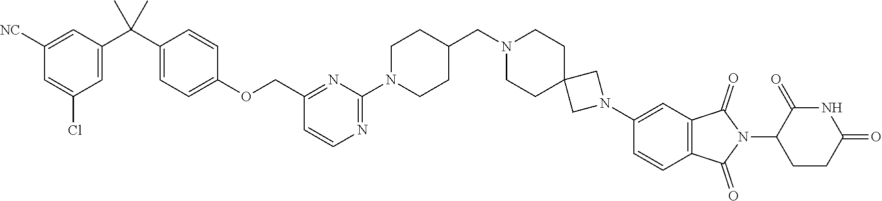 [SMILES: CC(C)(c1ccc(OCc2ccnc(N3CCC(CN4CCC5(CC4)CN(c4ccc6c(c4)C(=O)N(C4CCC(=O)NC4=O)C6=O)C5)CC3)n2)cc1)c1cc(Cl)cc(C#N)c1]

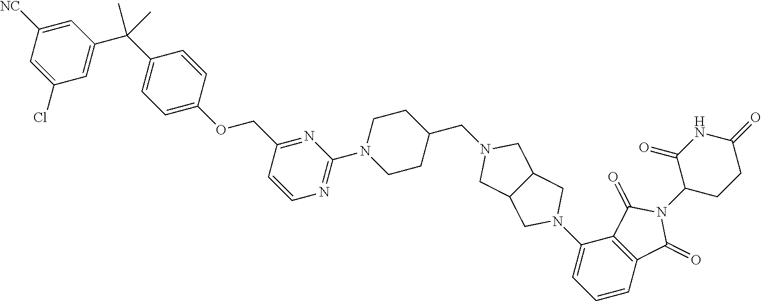 [SMILES: CC(C)(c1ccc(OCc2ccnc(N3CCC(CN4CC5CN(c6cccc7c6C(=O)N(C6CCC(=O)NC6=O)C7=O)CC5C4)CC3)n2)cc1)c1cc(Cl)cc(C#N)c1]

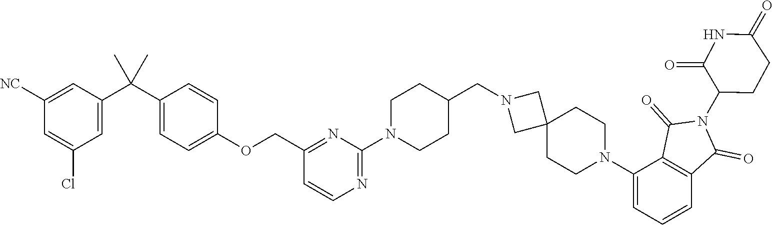 [SMILES: CC(C)(c1ccc(OCc2ccnc(N3CCC(CN4CC5(CCN(c6cccc7c6C(=O)N(C6CCC(=O)NC6=O)C7=O)CC5)C4)CC3)n2)cc1)c1cc(Cl)cc(C#N)c1]

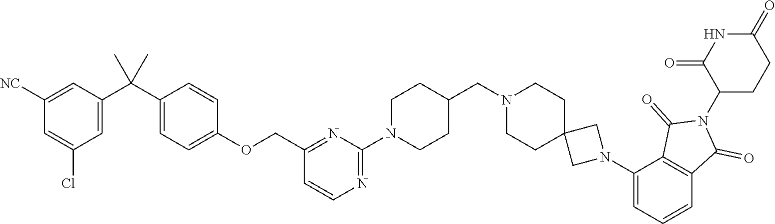 [SMILES: CC(C)(c1ccc(OCc2ccnc(N3CCC(CN4CCC5(CC4)CN(c4cccc6c4C(=O)N(C4CCC(=O)NC4=O)C6=O)C5)CC3)n2)cc1)c1cc(Cl)cc(C#N)c1]

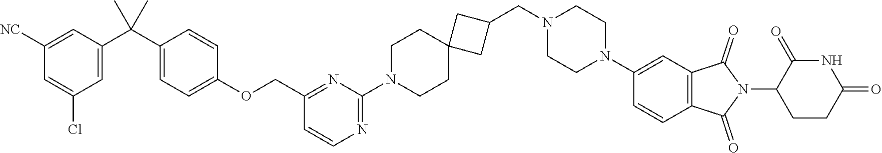 [SMILES: CC(C)(c1ccc(OCc2ccnc(N3CCC4(CC3)CC(CN3CCN(c5ccc6c(c5)C(=O)N(C5CCC(=O)NC5=O)C6=O)CC3)C4)n2)cc1)c1cc(Cl)cc(C#N)c1]

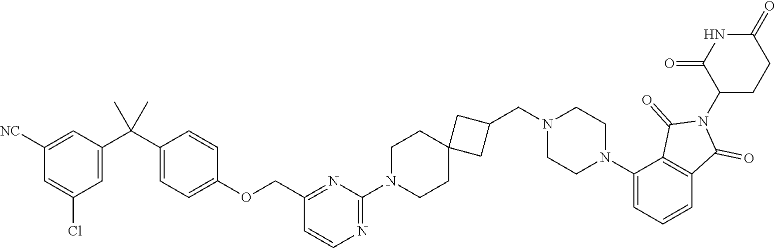 [SMILES: CC(C)(c1ccc(OCc2ccnc(N3CCC4(CC3)CC(CN3CCN(c5cccc6c5C(=O)N(C5CCC(=O)NC5=O)C6=O)CC3)C4)n2)cc1)c1cc(Cl)cc(C#N)c1]

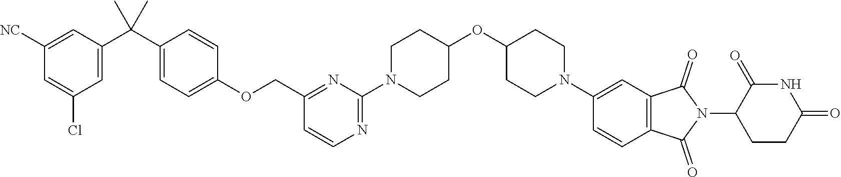 [SMILES: CC(C)(c1ccc(OCc2ccnc(N3CCC(OC4CCN(c5ccc6c(c5)C(=O)N(C5CCC(=O)NC5=O)C6=O)CC4)CC3)n2)cc1)c1cc(Cl)cc(C#N)c1]

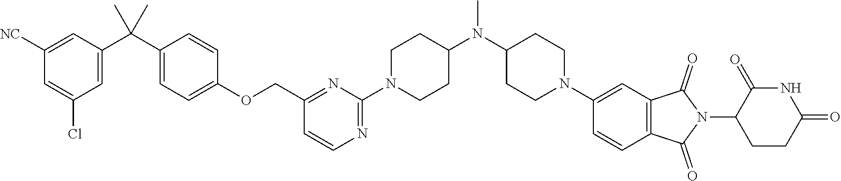 [SMILES: CN(C1CCN(c2ccc3c(c2)C(=O)N(C2CCC(=O)NC2=O)C3=O)CC1)C1CCN(c2nccc(COc3ccc(C(C)(C)c4cc(Cl)cc(C#N)c4)cc3)n2)CC1]

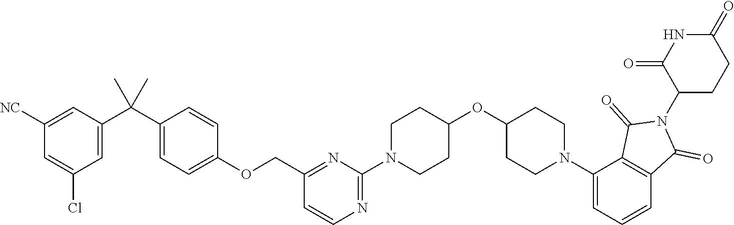 [SMILES: CC(C)(c1ccc(OCc2ccnc(N3CCC(OC4CCN(c5cccc6c5C(=O)N(C5CCC(=O)NC5=O)C6=O)CC4)CC3)n2)cc1)c1cc(Cl)cc(C#N)c1]

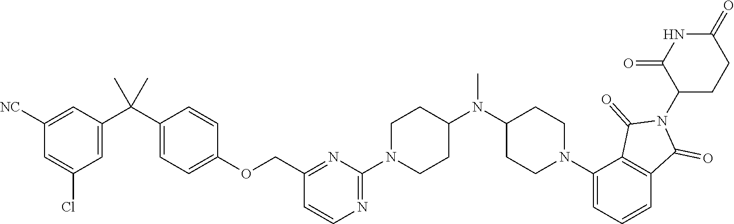 [SMILES: CN(C1CCN(c2nccc(COc3ccc(C(C)(C)c4cc(Cl)cc(C#N)c4)cc3)n2)CC1)C1CCN(c2cccc3c2C(=O)N(C2CCC(=O)NC2=O)C3=O)CC1]

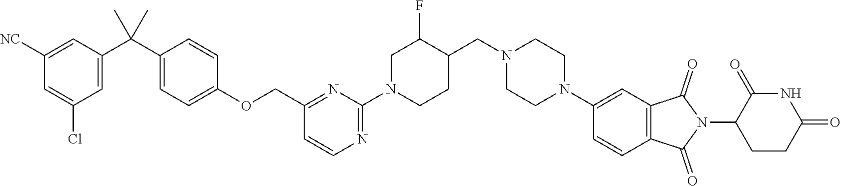 [SMILES: CC(C)(c1ccc(OCc2ccnc(N3CCC(CN4CCN(c5ccc6c(c5)C(=O)N(C5CCC(=O)NC5=O)C6=O)CC4)C(F)C3)n2)cc1)c1cc(Cl)cc(C#N)c1]

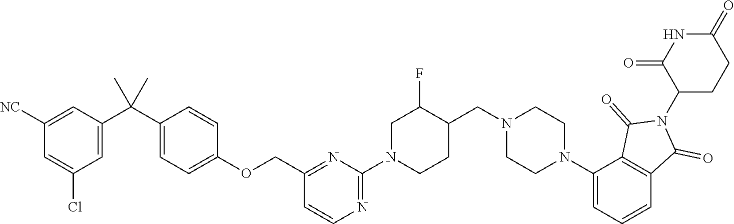 [SMILES: CC(C)(c1ccc(OCc2ccnc(N3CCC(CN4CCN(c5cccc6c5C(=O)N(C5CCC(=O)NC5=O)C6=O)CC4)C(F)C3)n2)cc1)c1cc(Cl)cc(C#N)c1]

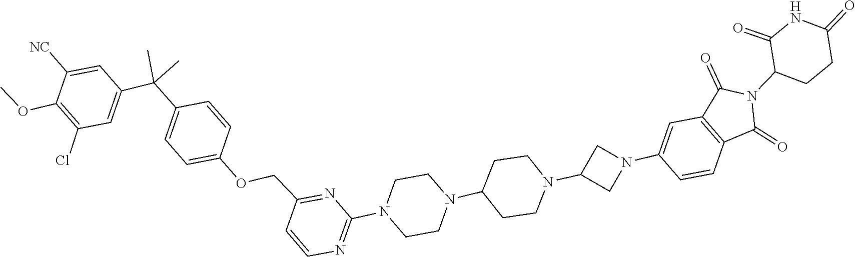 [SMILES: COc1c(Cl)cc(C(C)(C)c2ccc(OCc3ccnc(N4CCN(C5CCN(C6CN(c7ccc8c(c7)C(=O)N(C7CCC(=O)NC7=O)C8=O)C6)CC5)CC4)n3)cc2)cc1C#N]

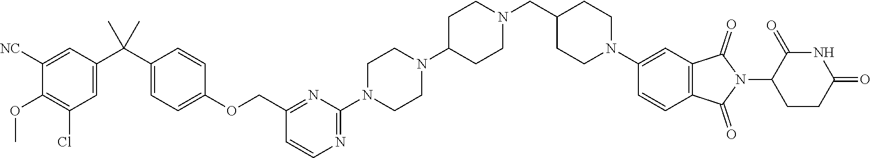 [SMILES: COc1c(Cl)cc(C(C)(C)c2ccc(OCc3ccnc(N4CCN(C5CCN(CC6CCN(c7ccc8c(c7)C(=O)N(C7CCC(=O)NC7=O)C8=O)CC6)CC5)CC4)n3)cc2)cc1C#N]

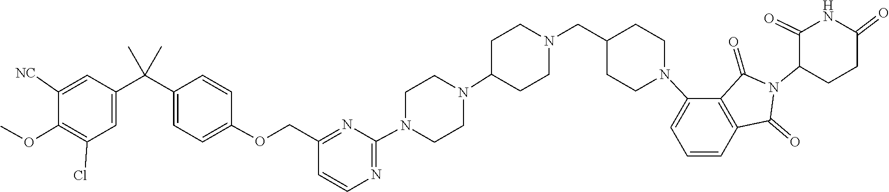 [SMILES: COc1c(Cl)cc(C(C)(C)c2ccc(OCc3ccnc(N4CCN(C5CCN(CC6CCN(c7cccc8c7C(=O)N(C7CCC(=O)NC7=O)C8=O)CC6)CC5)CC4)n3)cc2)cc1C#N]

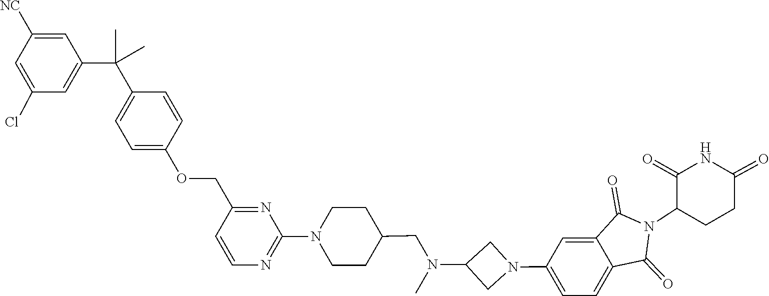 [SMILES: CN(CC1CCN(c2nccc(COc3ccc(C(C)(C)c4cc(Cl)cc(C#N)c4)cc3)n2)CC1)C1CN(c2ccc3c(c2)C(=O)N(C2CCC(=O)NC2=O)C3=O)C1]

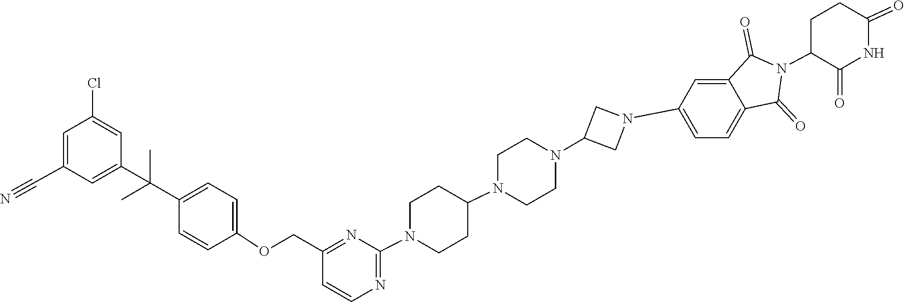 [SMILES: CC(C)(c1ccc(OCc2ccnc(N3CCC(N4CCN(C5CN(c6ccc7c(c6)C(=O)N(C6CCC(=O)NC6=O)C7=O)C5)CC4)CC3)n2)cc1)c1cc(Cl)cc(C#N)c1]

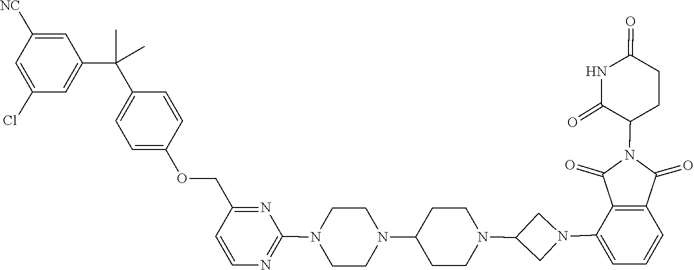 [SMILES: CC(C)(c1ccc(OCc2ccnc(N3CCN(C4CCN(C5CN(c6cccc7c6C(=O)N(C6CCC(=O)NC6=O)C7=O)C5)CC4)CC3)n2)cc1)c1cc(Cl)cc(C#N)c1]

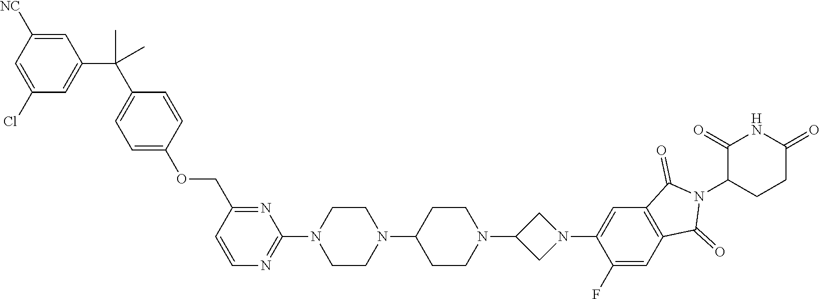 [SMILES: CC(C)(c1ccc(OCc2ccnc(N3CCN(C4CCN(C5CN(c6cc7c(cc6F)C(=O)N(C6CCC(=O)NC6=O)C7=O)C5)CC4)CC3)n2)cc1)c1cc(Cl)cc(C#N)c1]

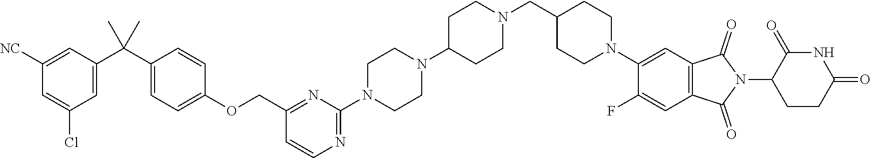 [SMILES: CC(C)(c1ccc(OCc2ccnc(N3CCN(C4CCN(CC5CCN(c6cc7c(cc6F)C(=O)N(C6CCC(=O)NC6=O)C7=O)CC5)CC4)CC3)n2)cc1)c1cc(Cl)cc(C#N)c1]

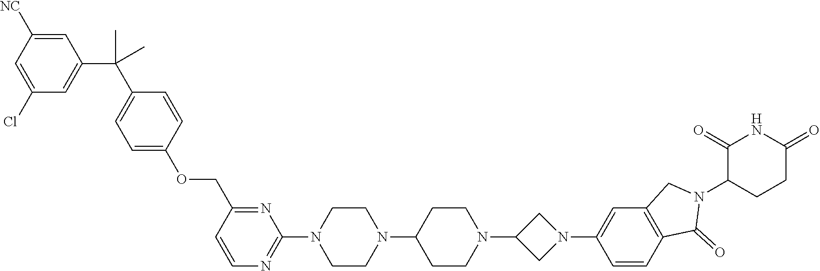 [SMILES: CC(C)(c1ccc(OCc2ccnc(N3CCN(C4CCN(C5CN(c6ccc7c(c6)CN(C6CCC(=O)NC6=O)C7=O)C5)CC4)CC3)n2)cc1)c1cc(Cl)cc(C#N)c1]

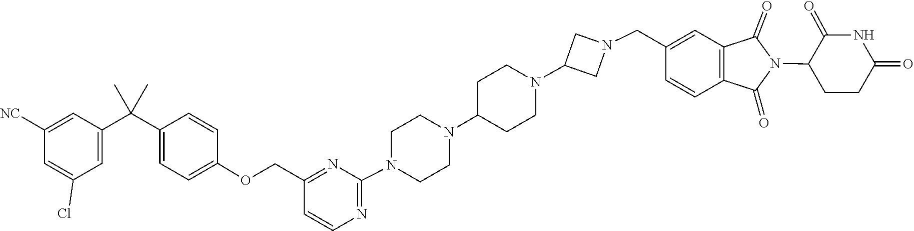 [SMILES: CC(C)(c1ccc(OCc2ccnc(N3CCN(C4CCN(C5CN(Cc6ccc7c(c6)C(=O)N(C6CCC(=O)NC6=O)C7=O)C5)CC4)CC3)n2)cc1)c1cc(Cl)cc(C#N)c1]

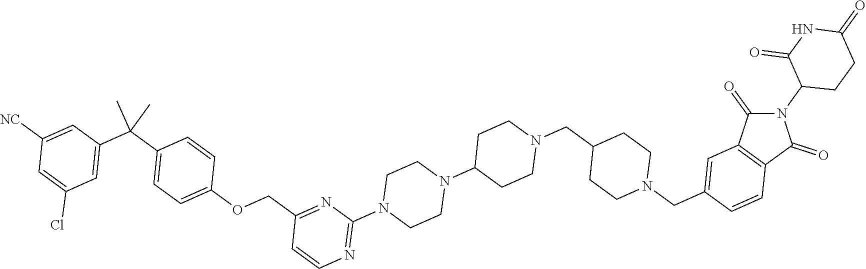 [SMILES: CC(C)(c1ccc(OCc2ccnc(N3CCN(C4CCN(CC5CCN(Cc6ccc7c(c6)C(=O)N(C6CCC(=O)NC6=O)C7=O)CC5)CC4)CC3)n2)cc1)c1cc(Cl)cc(C#N)c1]

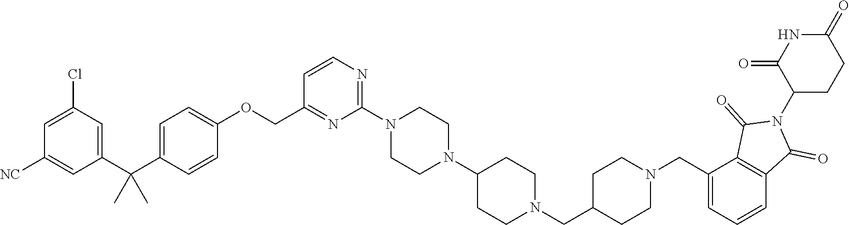 [SMILES: CC(C)(c1ccc(OCc2ccnc(N3CCN(C4CCN(CC5CCN(Cc6cccc7c6C(=O)N(C6CCC(=O)NC6=O)C7=O)CC5)CC4)CC3)n2)cc1)c1cc(Cl)cc(C#N)c1]

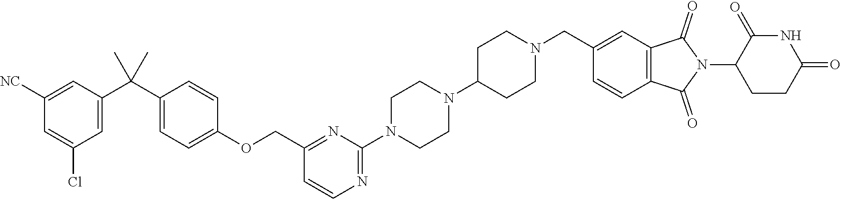 [SMILES: CC(C)(c1ccc(OCc2ccnc(N3CCN(C4CCN(Cc5ccc6c(c5)C(=O)N(C5CCC(=O)NC5=O)C6=O)CC4)CC3)n2)cc1)c1cc(Cl)cc(C#N)c1]

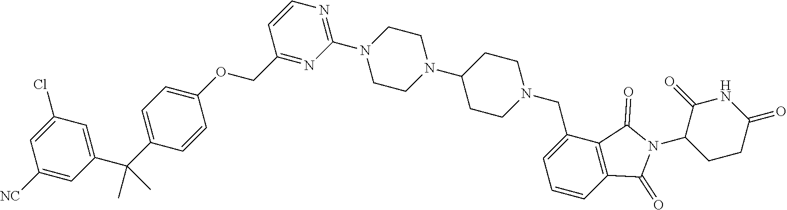 [SMILES: CC(C)(c1ccc(OCc2ccnc(N3CCN(C4CCN(Cc5cccc6c5C(=O)N(C5CCC(=O)NC5=O)C6=O)CC4)CC3)n2)cc1)c1cc(Cl)cc(C#N)c1]

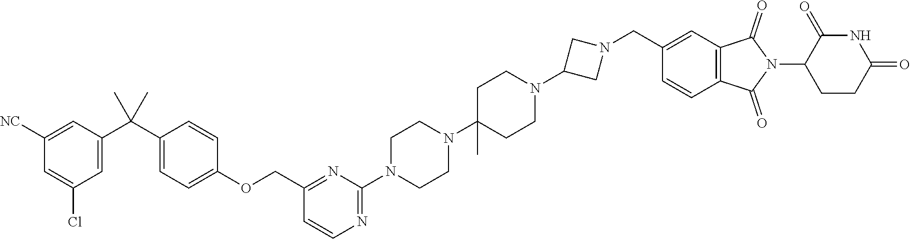 [SMILES: CC(C)(c1ccc(OCc2ccnc(N3CCN(C4(C)CCN(C5CN(Cc6ccc7c(c6)C(=O)N(C6CCC(=O)NC6=O)C7=O)C5)CC4)CC3)n2)cc1)c1cc(Cl)cc(C#N)c1]

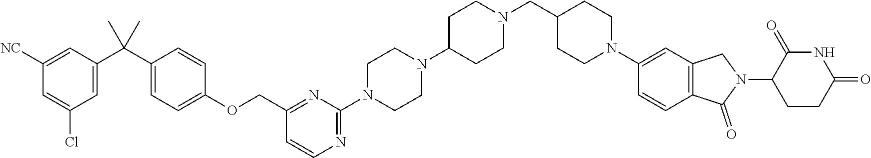 [SMILES: CC(C)(c1ccc(OCc2ccnc(N3CCN(C4CCN(CC5CCN(c6ccc7c(c6)CN(C6CCC(=O)NC6=O)C7=O)CC5)CC4)CC3)n2)cc1)c1cc(Cl)cc(C#N)c1]

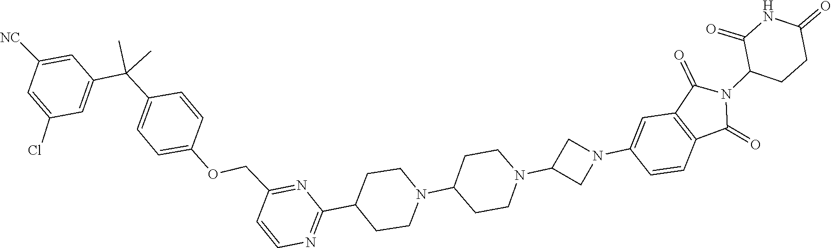 [SMILES: CC(C)(c1ccc(OCc2ccnc(C3CCN(C4CCN(C5CN(c6ccc7c(c6)C(=O)N(C6CCC(=O)NC6=O)C7=O)C5)CC4)CC3)n2)cc1)c1cc(Cl)cc(C#N)c1]

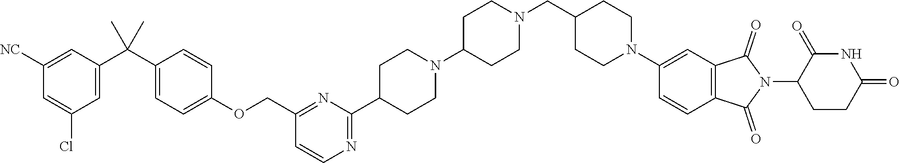 [SMILES: CC(C)(c1ccc(OCc2ccnc(C3CCN(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

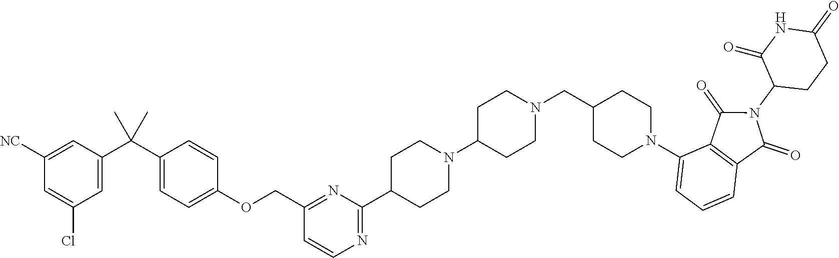 [SMILES: CC(C)(c1ccc(OCc2ccnc(C3CCN(C4CCN(CC5CCN(c6cccc7c6C(=O)N(C6CCC(=O)NC6=O)C7=O)CC5)CC4)CC3)n2)cc1)c1cc(Cl)cc(C#N)c1]

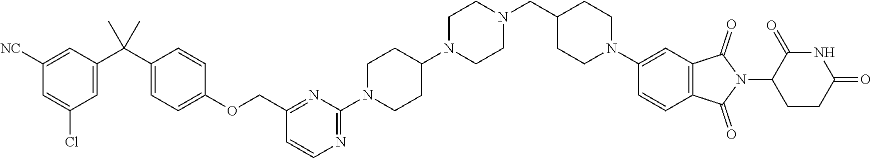 [SMILES: CC(C)(c1ccc(OCc2ccnc(N3CCC(N4CCN(CC5CCN(c6ccc7c(c6)C(=O)N(C6CCC(=O)NC6=O)C7=O)CC5)CC4)CC3)n2)cc1)c1cc(Cl)cc(C#N)c1]

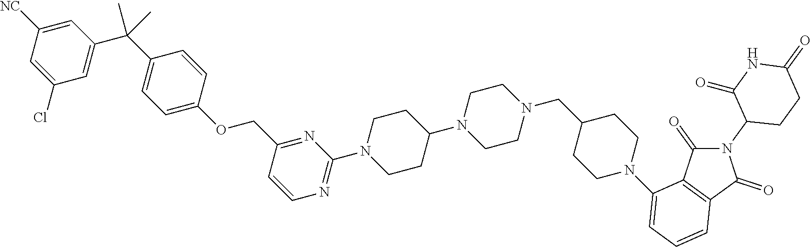 [SMILES: CC(C)(c1ccc(OCc2ccnc(N3CCC(N4CCN(CC5CCN(c6cccc7c6C(=O)N(C6CCC(=O)NC6=O)C7=O)CC5)CC4)CC3)n2)cc1)c1cc(Cl)cc(C#N)c1]

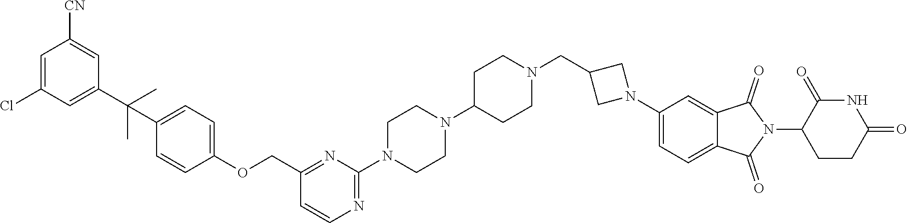 [SMILES: CC(C)(c1ccc(OCc2ccnc(N3CCN(C4CCN(CC5CN(c6ccc7c(c6)C(=O)N(C6CCC(=O)NC6=O)C7=O)C5)CC4)CC3)n2)cc1)c1cc(Cl)cc(C#N)c1]

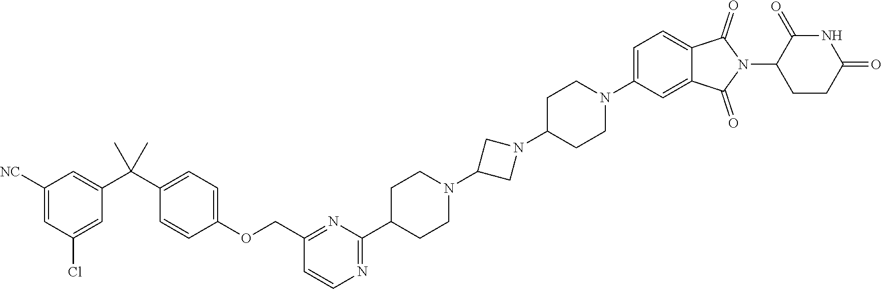 [SMILES: CC(C)(c1ccc(OCc2ccnc(C3CCN(C4CN(C5CCN(c6ccc7c(c6)C(=O)N(C6CCC(=O)NC6=O)C7=O)CC5)C4)CC3)n2)cc1)c1cc(Cl)cc(C#N)c1]

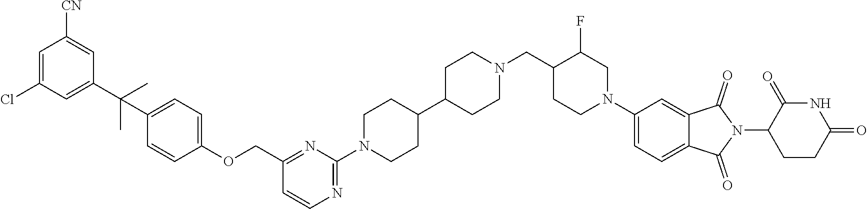 [SMILES: CC(C)(c1ccc(OCc2ccnc(N3CCC(C4CCN(CC5CCN(c6ccc7c(c6)C(=O)N(C6CCC(=O)NC6=O)C7=O)CC5F)CC4)CC3)n2)cc1)c1cc(Cl)cc(C#N)c1]

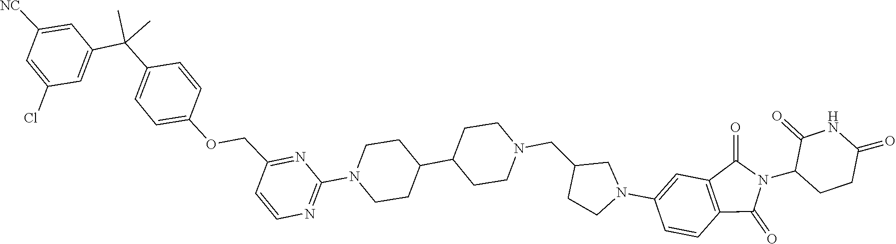 [SMILES: CC(C)(c1ccc(OCc2ccnc(N3CCC(C4CCN(CC5CCN(c6ccc7c(c6)C(=O)N(C6CCC(=O)NC6=O)C7=O)C5)CC4)CC3)n2)cc1)c1cc(Cl)cc(C#N)c1]

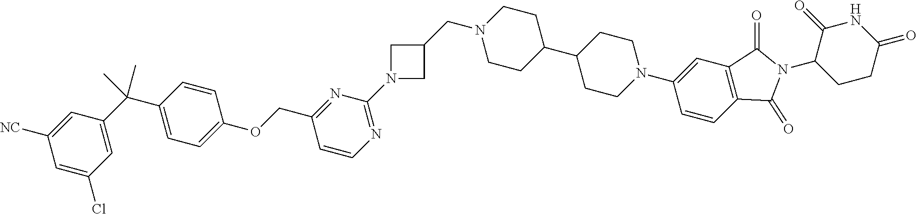 [SMILES: CC(C)(c1ccc(OCc2ccnc(N3CC(CN4CCC(C5CCN(c6ccc7c(c6)C(=O)N(C6CCC(=O)NC6=O)C7=O)CC5)CC4)C3)n2)cc1)c1cc(Cl)cc(C#N)c1]

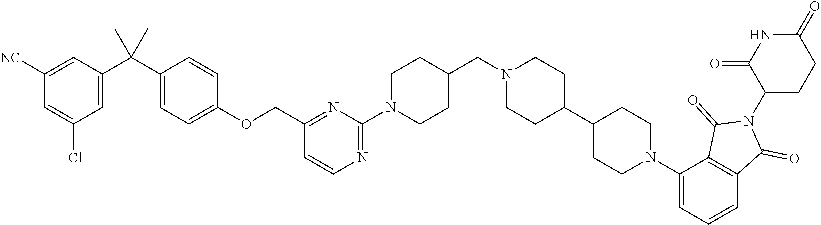 [SMILES: CC(C)(c1ccc(OCc2ccnc(N3CCC(CN4CCC(C5CCN(c6cccc7c6C(=O)N(C6CCC(=O)NC6=O)C7=O)CC5)CC4)CC3)n2)cc1)c1cc(Cl)cc(C#N)c1]

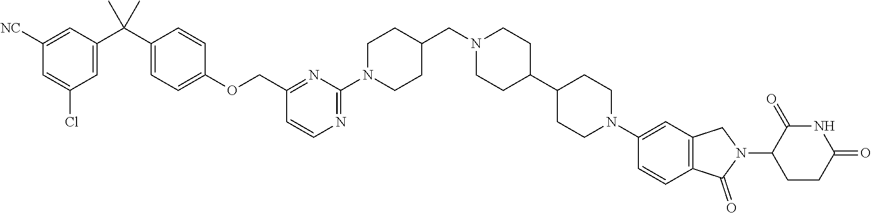 [SMILES: CC(C)(c1ccc(OCc2ccnc(N3CCC(CN4CCC(C5CCN(c6ccc7c(c6)CN(C6CCC(=O)NC6=O)C7=O)CC5)CC4)CC3)n2)cc1)c1cc(Cl)cc(C#N)c1]

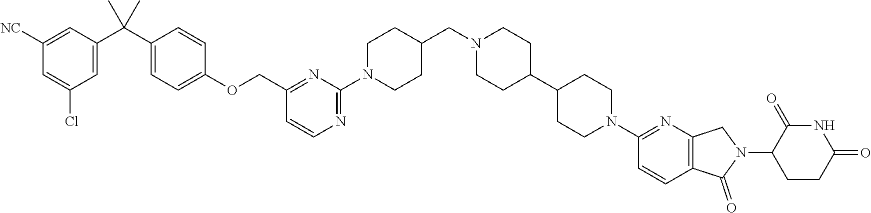 [SMILES: CC(C)(c1ccc(OCc2ccnc(N3CCC(CN4CCC(C5CCN(c6ccc7c(n6)CN(C6CCC(=O)NC6=O)C7=O)CC5)CC4)CC3)n2)cc1)c1cc(Cl)cc(C#N)c1]

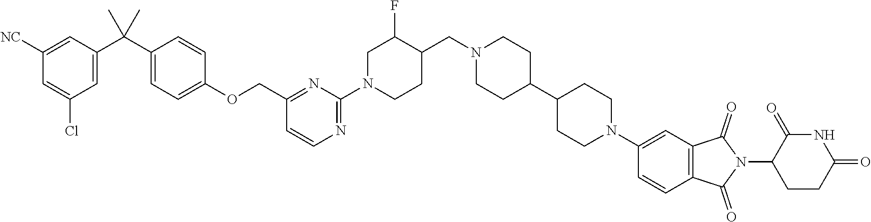 [SMILES: CC(C)(c1ccc(OCc2ccnc(N3CCC(CN4CCC(C5CCN(c6ccc7c(c6)C(=O)N(C6CCC(=O)NC6=O)C7=O)CC5)CC4)C(F)C3)n2)cc1)c1cc(Cl)cc(C#N)c1]

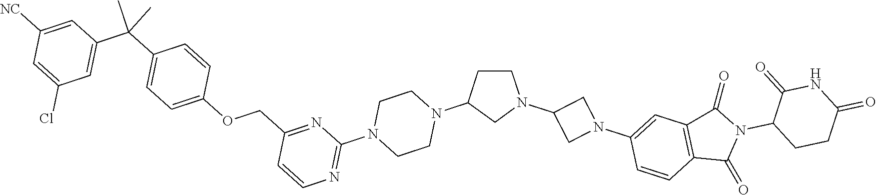 [SMILES: CC(C)(c1ccc(OCc2ccnc(N3CCN(C4CCN(C5CN(c6ccc7c(c6)C(=O)N(C6CCC(=O)NC6=O)C7=O)C5)C4)CC3)n2)cc1)c1cc(Cl)cc(C#N)c1]

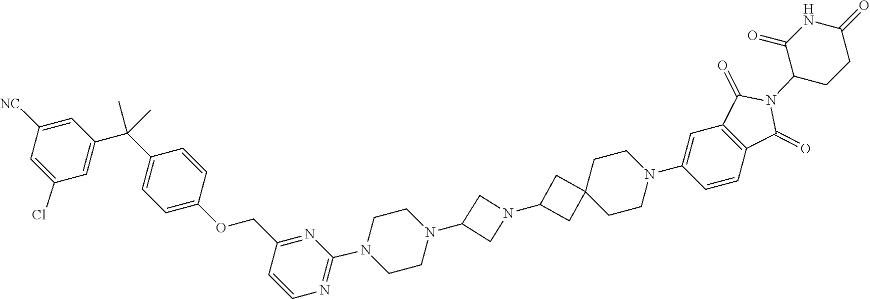 [SMILES: CC(C)(c1ccc(OCc2ccnc(N3CCN(C4CN(C5CC6(CCN(c7ccc8c(c7)C(=O)N(C7CCC(=O)NC7=O)C8=O)CC6)C5)C4)CC3)n2)cc1)c1cc(Cl)cc(C#N)c1]

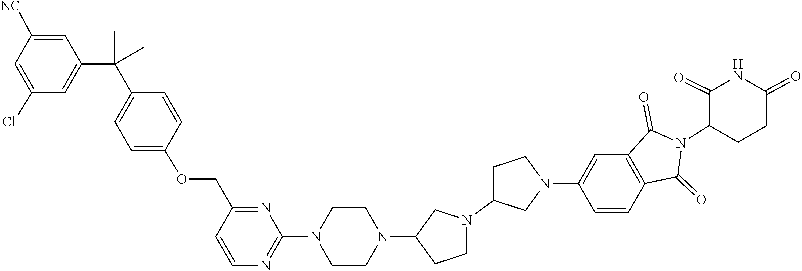 [SMILES: CC(C)(c1ccc(OCc2ccnc(N3CCN(C4CCN(C5CCN(c6ccc7c(c6)C(=O)N(C6CCC(=O)NC6=O)C7=O)C5)C4)CC3)n2)cc1)c1cc(Cl)cc(C#N)c1]

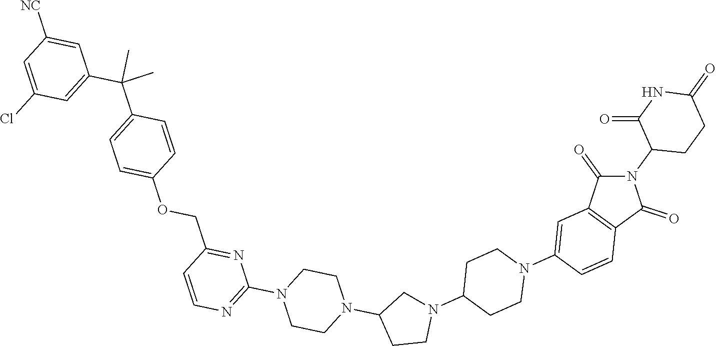 [SMILES: CC(C)(c1ccc(OCc2ccnc(N3CCN(C4CCN(C5CCN(c6ccc7c(c6)C(=O)N(C6CCC(=O)NC6=O)C7=O)CC5)C4)CC3)n2)cc1)c1cc(Cl)cc(C#N)c1]

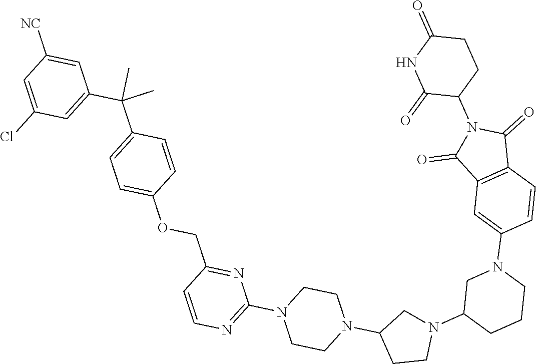 [SMILES: CC(C)(c1ccc(OCc2ccnc(N3CCN(C4CCN(C5CCCN(c6ccc7c(c6)C(=O)N(C6CCC(=O)NC6=O)C7=O)C5)C4)CC3)n2)cc1)c1cc(Cl)cc(C#N)c1]

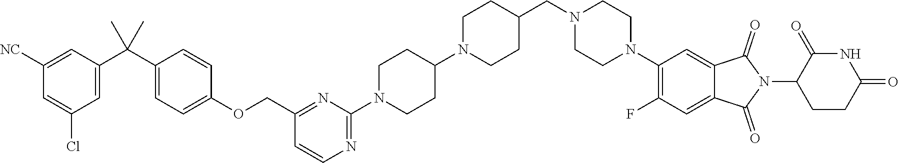 [SMILES: CC(C)(c1ccc(OCc2ccnc(N3CCC(N4CCC(CN5CCN(c6cc7c(cc6F)C(=O)N(C6CCC(=O)NC6=O)C7=O)CC5)CC4)CC3)n2)cc1)c1cc(Cl)cc(C#N)c1]

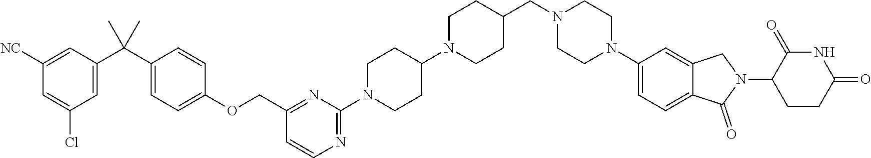 [SMILES: CC(C)(c1ccc(OCc2ccnc(N3CCC(N4CCC(CN5CCN(c6ccc7c(c6)CN(C6CCC(=O)NC6=O)C7=O)CC5)CC4)CC3)n2)cc1)c1cc(Cl)cc(C#N)c1]

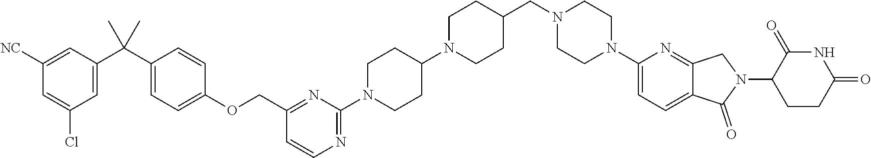 [SMILES: CC(C)(c1ccc(OCc2ccnc(N3CCC(N4CCC(CN5CCN(c6ccc7c(n6)CN(C6CCC(=O)NC6=O)C7=O)CC5)CC4)CC3)n2)cc1)c1cc(Cl)cc(C#N)c1]

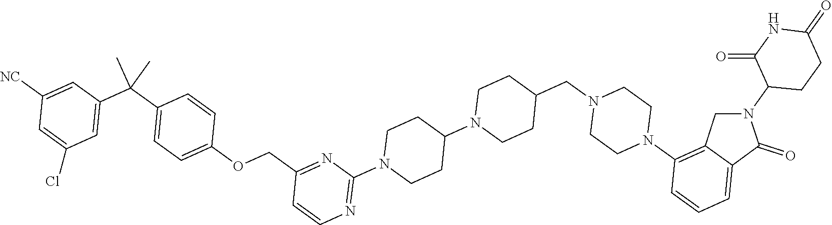 [SMILES: CC(C)(c1ccc(OCc2ccnc(N3CCC(N4CCC(CN5CCN(c6cccc7c6CN(C6CCC(=O)NC6=O)C7=O)CC5)CC4)CC3)n2)cc1)c1cc(Cl)cc(C#N)c1]

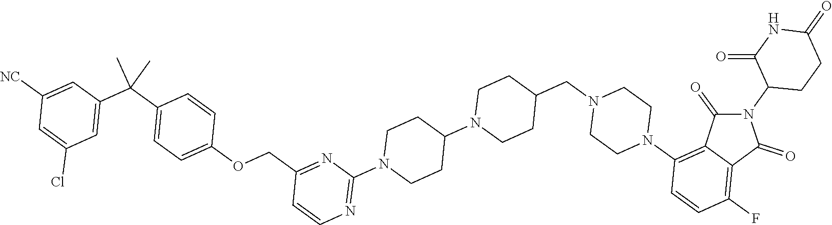 [SMILES: CC(C)(c1ccc(OCc2ccnc(N3CCC(N4CCC(CN5CCN(c6ccc(F)c7c6C(=O)N(C6CCC(=O)NC6=O)C7=O)CC5)CC4)CC3)n2)cc1)c1cc(Cl)cc(C#N)c1]

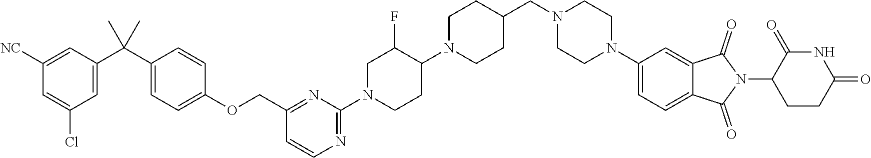 [SMILES: CC(C)(c1ccc(OCc2ccnc(N3CCC(N4CCC(CN5CCN(c6ccc7c(c6)C(=O)N(C6CCC(=O)NC6=O)C7=O)CC5)CC4)C(F)C3)n2)cc1)c1cc(Cl)cc(C#N)c1]

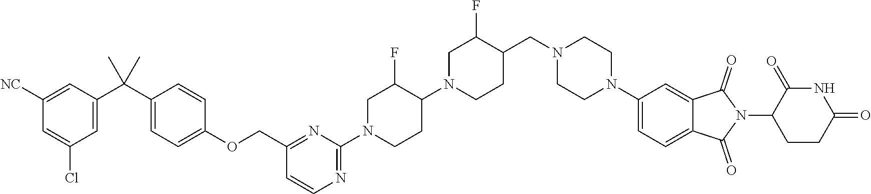 [SMILES: CC(C)(c1ccc(OCc2ccnc(N3CCC(N4CCC(CN5CCN(c6ccc7c(c6)C(=O)N(C6CCC(=O)NC6=O)C7=O)CC5)C(F)C4)C(F)C3)n2)cc1)c1cc(Cl)cc(C#N)c1]

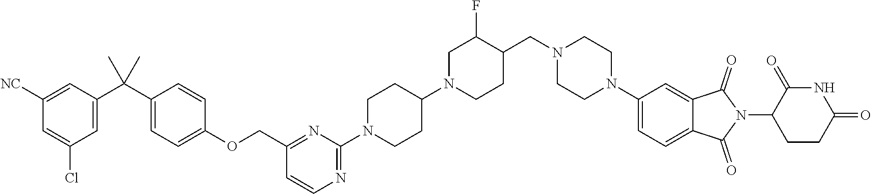 [SMILES: CC(C)(c1ccc(OCc2ccnc(N3CCC(N4CCC(CN5CCN(c6ccc7c(c6)C(=O)N(C6CCC(=O)NC6=O)C7=O)CC5)C(F)C4)CC3)n2)cc1)c1cc(Cl)cc(C#N)c1]

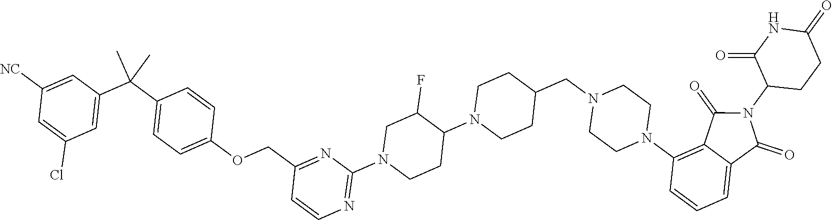 [SMILES: CC(C)(c1ccc(OCc2ccnc(N3CCC(N4CCC(CN5CCN(c6cccc7c6C(=O)N(C6CCC(=O)NC6=O)C7=O)CC5)CC4)C(F)C3)n2)cc1)c1cc(Cl)cc(C#N)c1]

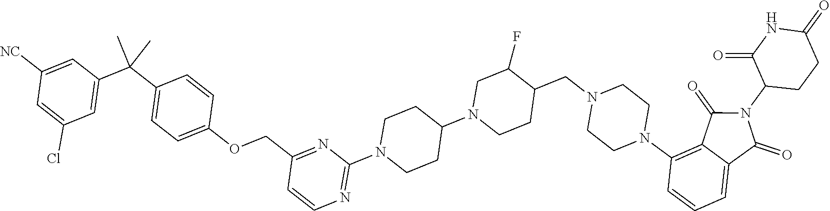 [SMILES: CC(C)(c1ccc(OCc2ccnc(N3CCC(N4CCC(CN5CCN(c6cccc7c6C(=O)N(C6CCC(=O)NC6=O)C7=O)CC5)C(F)C4)CC3)n2)cc1)c1cc(Cl)cc(C#N)c1]

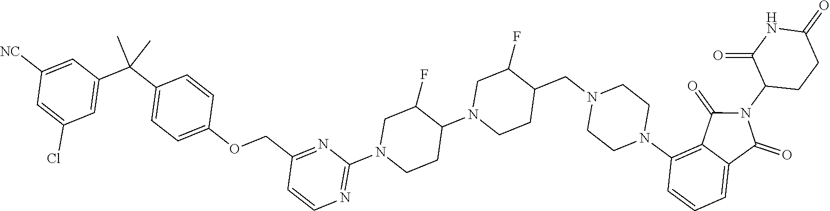 [SMILES: CC(C)(c1ccc(OCc2ccnc(N3CCC(N4CCC(CN5CCN(c6cccc7c6C(=O)N(C6CCC(=O)NC6=O)C7=O)CC5)C(F)C4)C(F)C3)n2)cc1)c1cc(Cl)cc(C#N)c1]

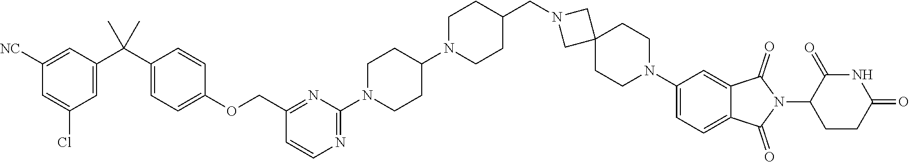 [SMILES: CC(C)(c1ccc(OCc2ccnc(N3CCC(N4CCC(CN5CC6(CCN(c7ccc8c(c7)C(=O)N(C7CCC(=O)NC7=O)C8=O)CC6)C5)CC4)CC3)n2)cc1)c1cc(Cl)cc(C#N)c1]

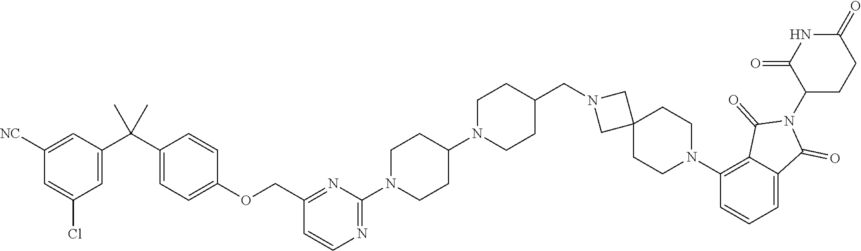 [SMILES: CC(C)(c1ccc(OCc2ccnc(N3CCC(N4CCC(CN5CC6(CCN(c7cccc8c7C(=O)N(C7CCC(=O)NC7=O)C8=O)CC6)C5)CC4)CC3)n2)cc1)c1cc(Cl)cc(C#N)c1]

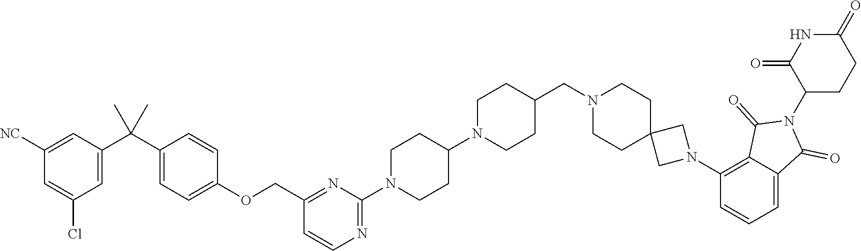 [SMILES: CC(C)(c1ccc(OCc2ccnc(N3CCC(N4CCC(CN5CCC6(CC5)CN(c5cccc7c5C(=O)N(C5CCC(=O)NC5=O)C7=O)C6)CC4)CC3)n2)cc1)c1cc(Cl)cc(C#N)c1]

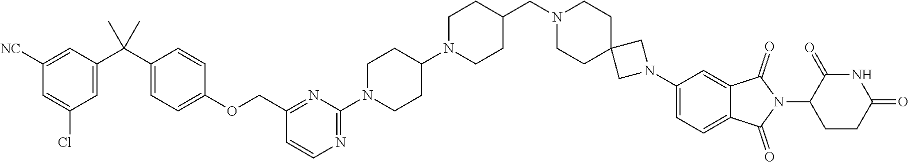 [SMILES: CC(C)(c1ccc(OCc2ccnc(N3CCC(N4CCC(CN5CCC6(CC5)CN(c5ccc7c(c5)C(=O)N(C5CCC(=O)NC5=O)C7=O)C6)CC4)CC3)n2)cc1)c1cc(Cl)cc(C#N)c1]

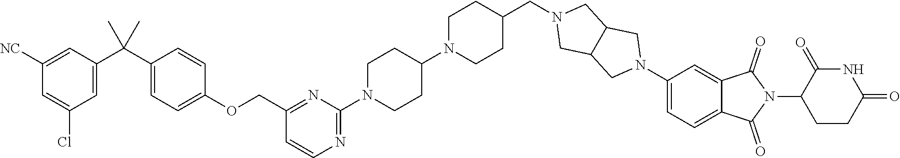 [SMILES: CC(C)(c1ccc(OCc2ccnc(N3CCC(N4CCC(CN5CC6CN(c7ccc8c(c7)C(=O)N(C7CCC(=O)NC7=O)C8=O)CC6C5)CC4)CC3)n2)cc1)c1cc(Cl)cc(C#N)c1]

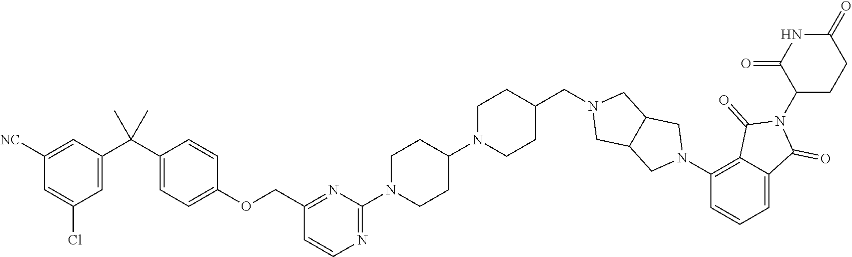 [SMILES: CC(C)(c1ccc(OCc2ccnc(N3CCC(N4CCC(CN5CC6CN(c7cccc8c7C(=O)N(C7CCC(=O)NC7=O)C8=O)CC6C5)CC4)CC3)n2)cc1)c1cc(Cl)cc(C#N)c1]

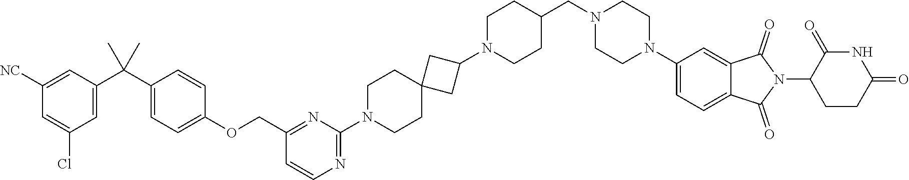 [SMILES: CC(C)(c1ccc(OCc2ccnc(N3CCC4(CC3)CC(N3CCC(CN5CCN(c6ccc7c(c6)C(=O)N(C6CCC(=O)NC6=O)C7=O)CC5)CC3)C4)n2)cc1)c1cc(Cl)cc(C#N)c1]

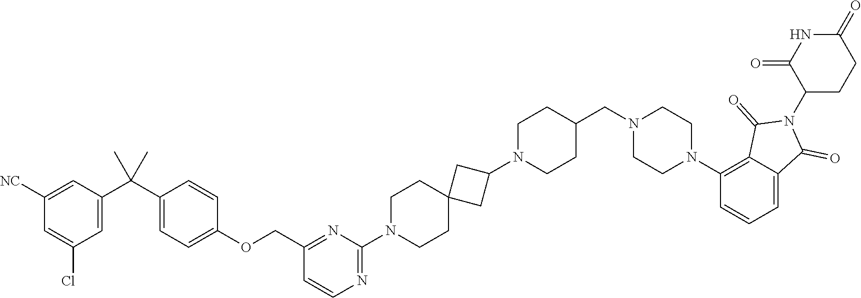 [SMILES: CC(C)(c1ccc(OCc2ccnc(N3CCC4(CC3)CC(N3CCC(CN5CCN(c6cccc7c6C(=O)N(C6CCC(=O)NC6=O)C7=O)CC5)CC3)C4)n2)cc1)c1cc(Cl)cc(C#N)c1]

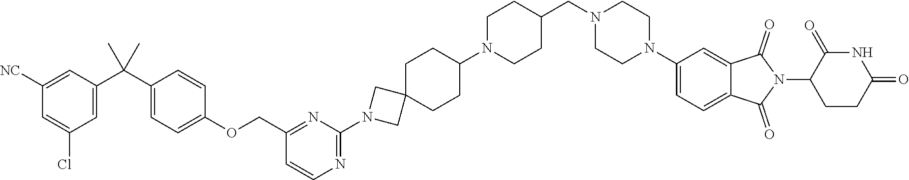 [SMILES: CC(C)(c1ccc(OCc2ccnc(N3CC4(CCC(N5CCC(CN6CCN(c7ccc8c(c7)C(=O)N(C7CCC(=O)NC7=O)C8=O)CC6)CC5)CC4)C3)n2)cc1)c1cc(Cl)cc(C#N)c1]

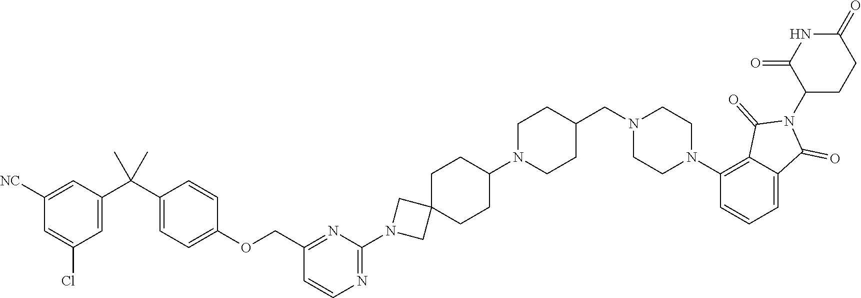 [SMILES: CC(C)(c1ccc(OCc2ccnc(N3CC4(CCC(N5CCC(CN6CCN(c7cccc8c7C(=O)N(C7CCC(=O)NC7=O)C8=O)CC6)CC5)CC4)C3)n2)cc1)c1cc(Cl)cc(C#N)c1]

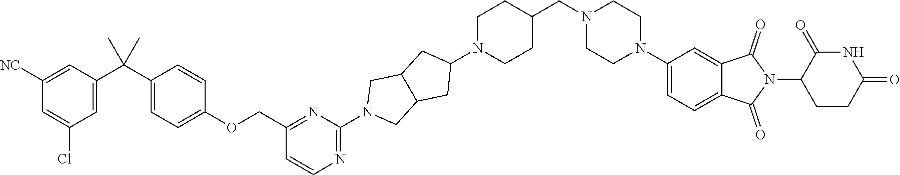 [SMILES: CC(C)(c1ccc(OCc2ccnc(N3CC4CC(N5CCC(CN6CCN(c7ccc8c(c7)C(=O)N(C7CCC(=O)NC7=O)C8=O)CC6)CC5)CC4C3)n2)cc1)c1cc(Cl)cc(C#N)c1]

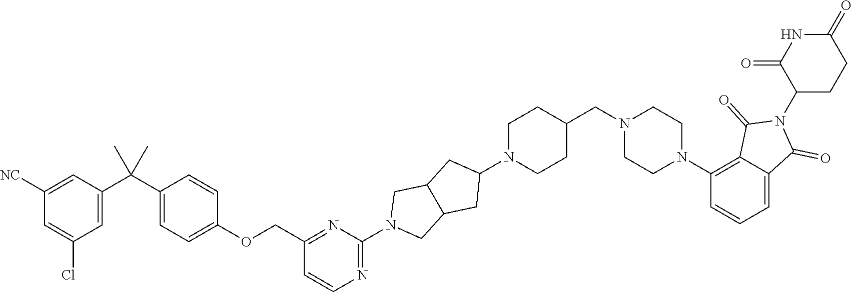 [SMILES: CC(C)(c1ccc(OCc2ccnc(N3CC4CC(N5CCC(CN6CCN(c7cccc8c7C(=O)N(C7CCC(=O)NC7=O)C8=O)CC6)CC5)CC4C3)n2)cc1)c1cc(Cl)cc(C#N)c1]

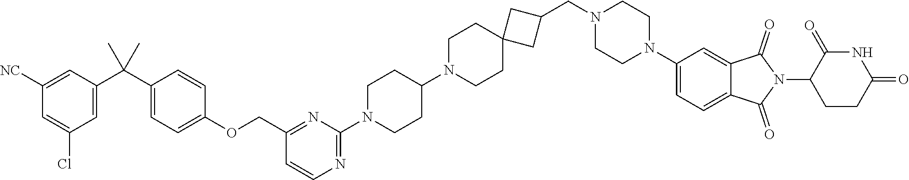 [SMILES: CC(C)(c1ccc(OCc2ccnc(N3CCC(N4CCC5(CC4)CC(CN4CCN(c6ccc7c(c6)C(=O)N(C6CCC(=O)NC6=O)C7=O)CC4)C5)CC3)n2)cc1)c1cc(Cl)cc(C#N)c1]

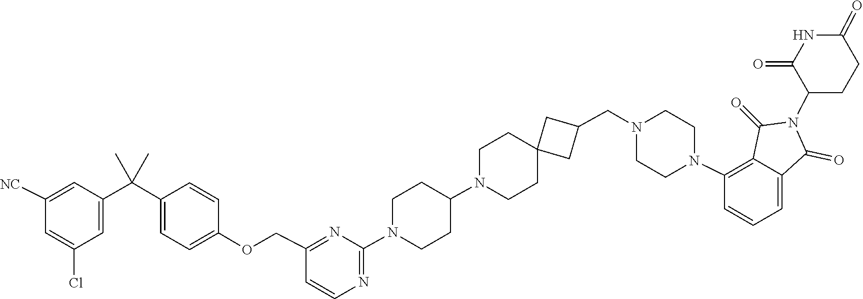 [SMILES: CC(C)(c1ccc(OCc2ccnc(N3CCC(N4CCC5(CC4)CC(CN4CCN(c6cccc7c6C(=O)N(C6CCC(=O)NC6=O)C7=O)CC4)C5)CC3)n2)cc1)c1cc(Cl)cc(C#N)c1]

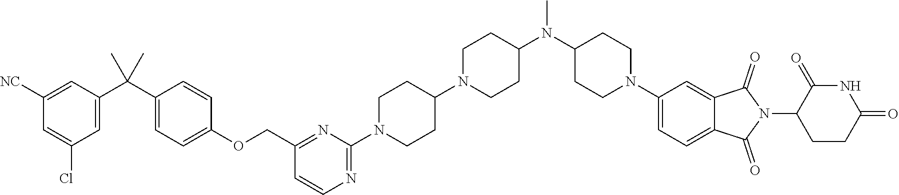 [SMILES: CN(C1CCN(c2ccc3c(c2)C(=O)N(C2CCC(=O)NC2=O)C3=O)CC1)C1CCN(C2CCN(c3nccc(COc4ccc(C(C)(C)c5cc(Cl)cc(C#N)c5)cc4)n3)CC2)CC1]

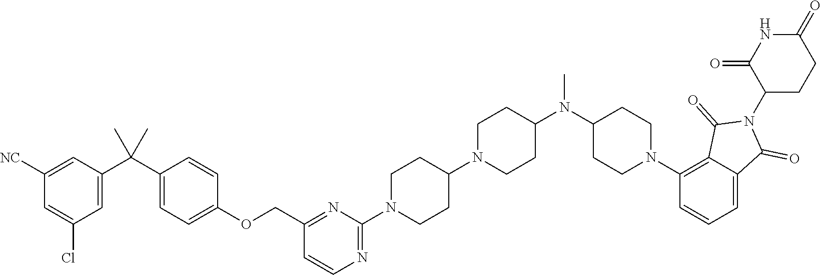 [SMILES: CN(C1CCN(c2cccc3c2C(=O)N(C2CCC(=O)NC2=O)C3=O)CC1)C1CCN(C2CCN(c3nccc(COc4ccc(C(C)(C)c5cc(Cl)cc(C#N)c5)cc4)n3)CC2)CC1]

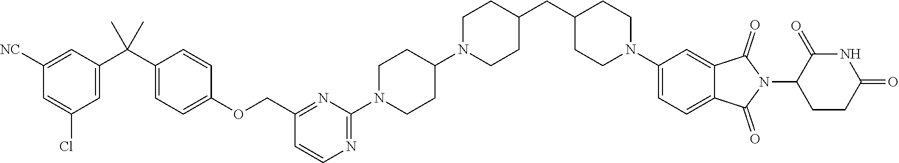 [SMILES: CC(C)(c1ccc(OCc2ccnc(N3CCC(N4CCC(CC5CCN(c6ccc7c(c6)C(=O)N(C6CCC(=O)NC6=O)C7=O)CC5)CC4)CC3)n2)cc1)c1cc(Cl)cc(C#N)c1]

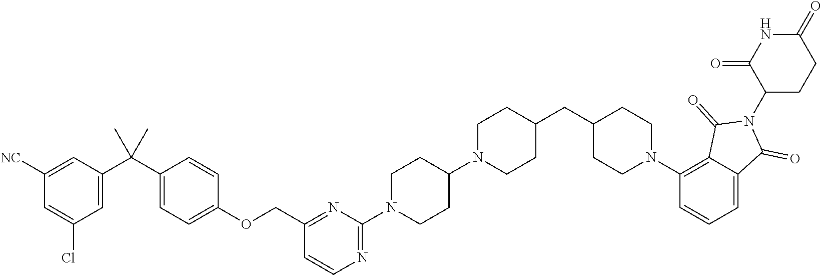 [SMILES: CC(C)(c1ccc(OCc2ccnc(N3CCC(N4CCC(CC5CCN(c6cccc7c6C(=O)N(C6CCC(=O)NC6=O)C7=O)CC5)CC4)CC3)n2)cc1)c1cc(Cl)cc(C#N)c1]

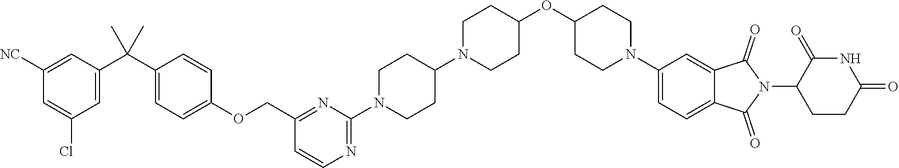 [SMILES: CC(C)(c1ccc(OCc2ccnc(N3CCC(N4CCC(OC5CCN(c6ccc7c(c6)C(=O)N(C6CCC(=O)NC6=O)C7=O)CC5)CC4)CC3)n2)cc1)c1cc(Cl)cc(C#N)c1]

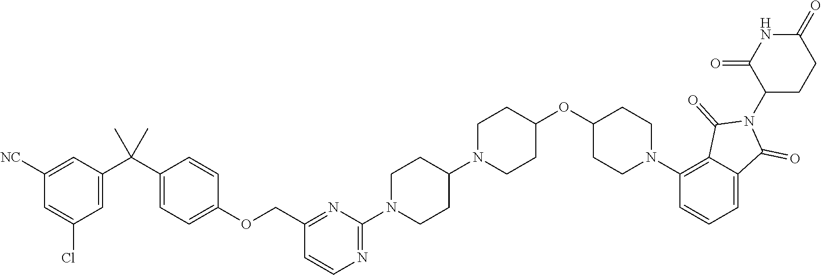 [SMILES: CC(C)(c1ccc(OCc2ccnc(N3CCC(N4CCC(OC5CCN(c6cccc7c6C(=O)N(C6CCC(=O)NC6=O)C7=O)CC5)CC4)CC3)n2)cc1)c1cc(Cl)cc(C#N)c1]

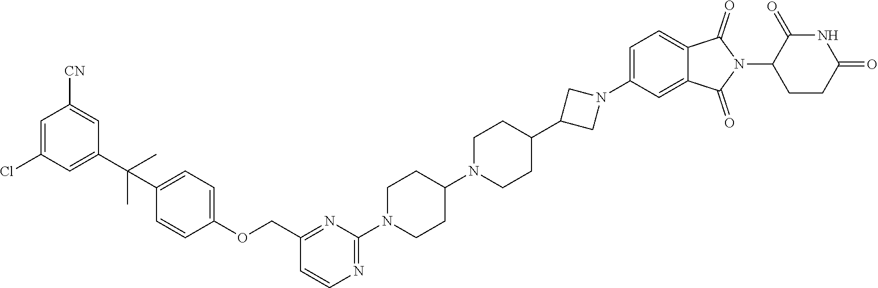 [SMILES: CC(C)(c1ccc(OCc2ccnc(N3CCC(N4CCC(C5CN(c6ccc7c(c6)C(=O)N(C6CCC(=O)NC6=O)C7=O)C5)CC4)CC3)n2)cc1)c1cc(Cl)cc(C#N)c1]

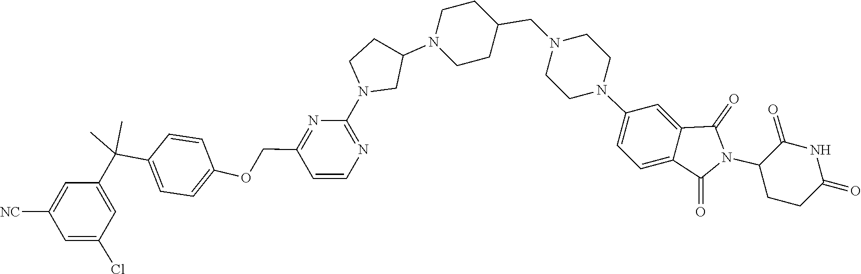 [SMILES: CC(C)(c1ccc(OCc2ccnc(N3CCC(N4CCC(CN5CCN(c6ccc7c(c6)C(=O)N(C6CCC(=O)NC6=O)C7=O)CC5)CC4)C3)n2)cc1)c1cc(Cl)cc(C#N)c1]

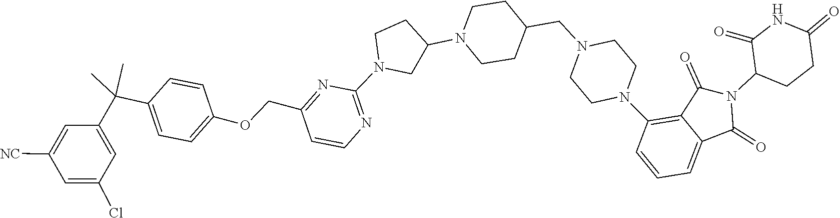 [SMILES: CC(C)(c1ccc(OCc2ccnc(N3CCC(N4CCC(CN5CCN(c6cccc7c6C(=O)N(C6CCC(=O)NC6=O)C7=O)CC5)CC4)C3)n2)cc1)c1cc(Cl)cc(C#N)c1]

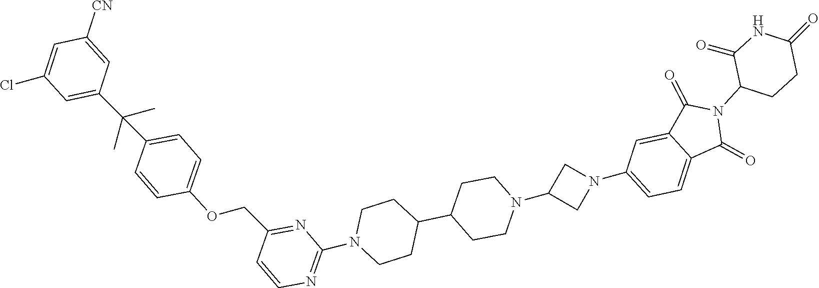 [SMILES: CC(C)(c1ccc(OCc2ccnc(N3CCC(C4CCN(C5CN(c6ccc7c(c6)C(=O)N(C6CCC(=O)NC6=O)C7=O)C5)CC4)CC3)n2)cc1)c1cc(Cl)cc(C#N)c1]

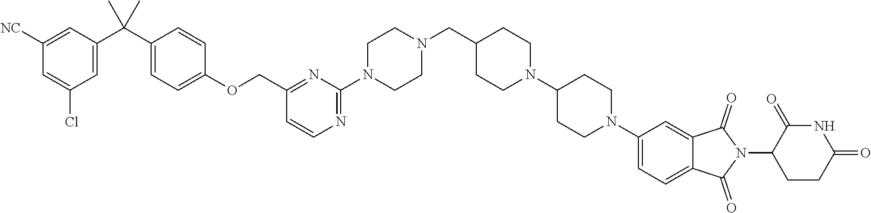 [SMILES: CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)cc(C#N)c1]

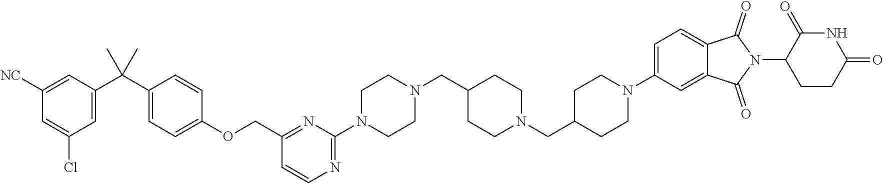 [SMILES: CC(C)(c1ccc(OCc2ccnc(N3CCN(CC4CCN(CC5CCN(c6ccc7c(c6)C(=O)N(C6CCC(=O)NC6=O)C7=O)CC5)CC4)CC3)n2)cc1)c1cc(Cl)cc(C#N)c1]

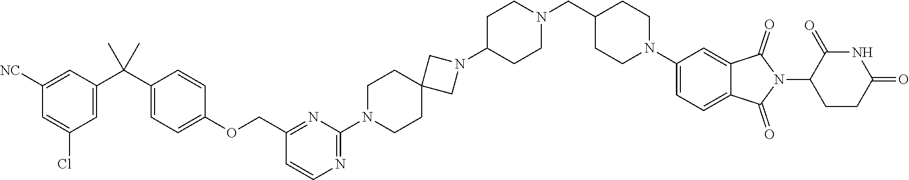 [SMILES: CC(C)(c1ccc(OCc2ccnc(N3CCC4(CC3)CN(C3CCN(CC5CCN(c6ccc7c(c6)C(=O)N(C6CCC(=O)NC6=O)C7=O)CC5)CC3)C4)n2)cc1)c1cc(Cl)cc(C#N)c1]

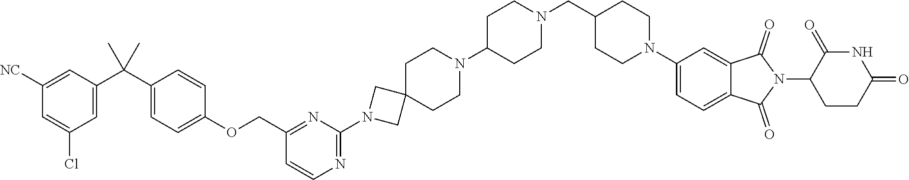 [SMILES: CC(C)(c1ccc(OCc2ccnc(N3CC4(CCN(C5CCN(CC6CCN(c7ccc8c(c7)C(=O)N(C7CCC(=O)NC7=O)C8=O)CC6)CC5)CC4)C3)n2)cc1)c1cc(Cl)cc(C#N)c1]

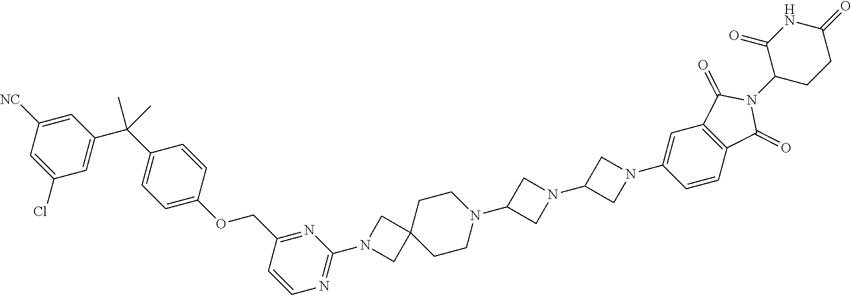 [SMILES: CC(C)(c1ccc(OCc2ccnc(N3CC4(CCN(C5CN(C6CN(c7ccc8c(c7)C(=O)N(C7CCC(=O)NC7=O)C8=O)C6)C5)CC4)C3)n2)cc1)c1cc(Cl)cc(C#N)c1]

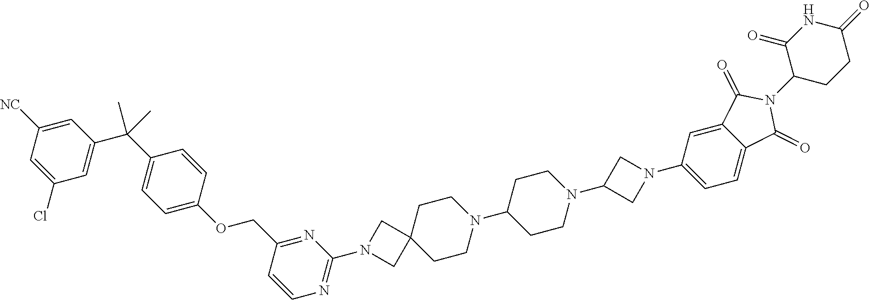 [SMILES: CC(C)(c1ccc(OCc2ccnc(N3CC4(CCN(C5CCN(C6CN(c7ccc8c(c7)C(=O)N(C7CCC(=O)NC7=O)C8=O)C6)CC5)CC4)C3)n2)cc1)c1cc(Cl)cc(C#N)c1]

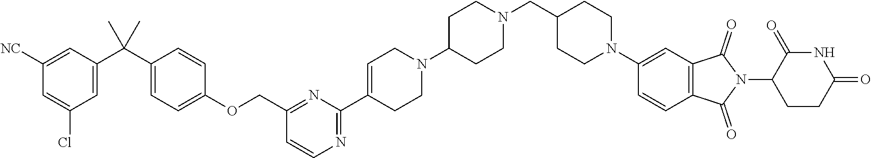 [SMILES: CC(C)(c1ccc(OCc2ccnc(C3=CCN(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

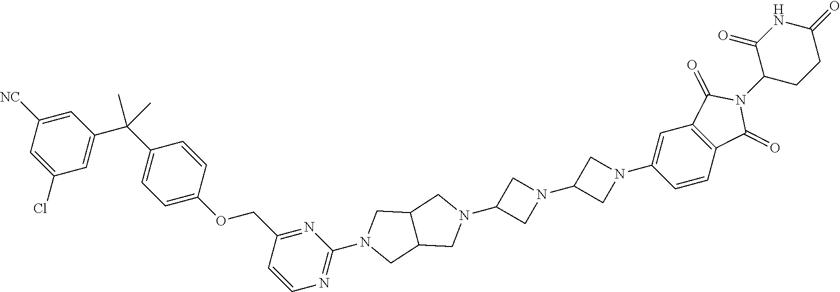 [SMILES: CC(C)(c1ccc(OCc2ccnc(N3CC4CN(C5CN(C6CN(c7ccc8c(c7)C(=O)N(C7CCC(=O)NC7=O)C8=O)C6)C5)CC4C3)n2)cc1)c1cc(Cl)cc(C#N)c1]

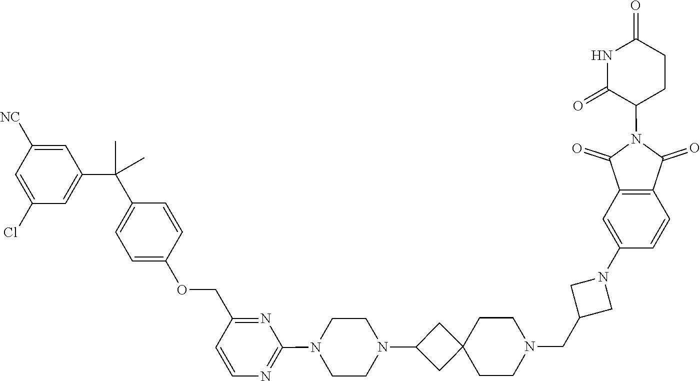 [SMILES: CC(C)(c1ccc(OCc2ccnc(N3CCN(C4CC5(CCN(CC6CN(c7ccc8c(c7)C(=O)N(C7CCC(=O)NC7=O)C8=O)C6)CC5)C4)CC3)n2)cc1)c1cc(Cl)cc(C#N)c1]

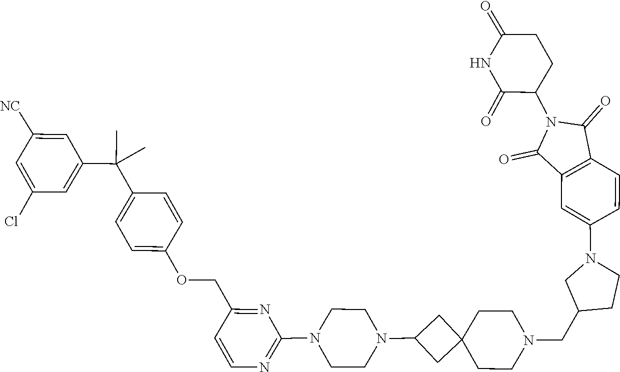 [SMILES: CC(C)(c1ccc(OCc2ccnc(N3CCN(C4CC5(CCN(CC6CCN(c7ccc8c(c7)C(=O)N(C7CCC(=O)NC7=O)C8=O)C6)CC5)C4)CC3)n2)cc1)c1cc(Cl)cc(C#N)c1]

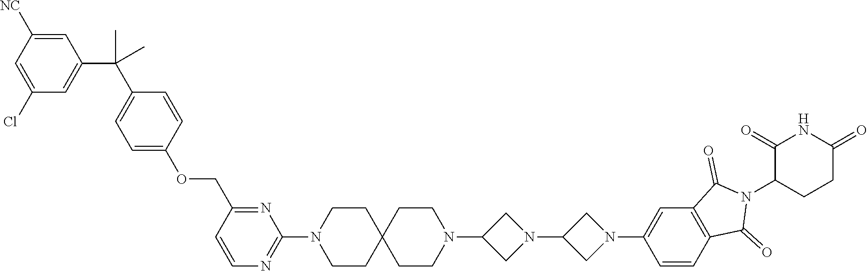 [SMILES: CC(C)(c1ccc(OCc2ccnc(N3CCC4(CC3)CCN(C3CN(C5CN(c6ccc7c(c6)C(=O)N(C6CCC(=O)NC6=O)C7=O)C5)C3)CC4)n2)cc1)c1cc(Cl)cc(C#N)c1]

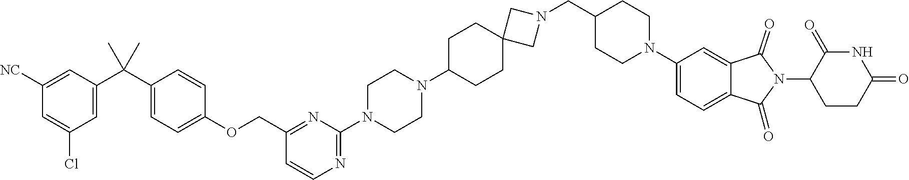 [SMILES: CC(C)(c1ccc(OCc2ccnc(N3CCN(C4CCC5(CC4)CN(CC4CCN(c6ccc7c(c6)C(=O)N(C6CCC(=O)NC6=O)C7=O)CC4)C5)CC3)n2)cc1)c1cc(Cl)cc(C#N)c1]

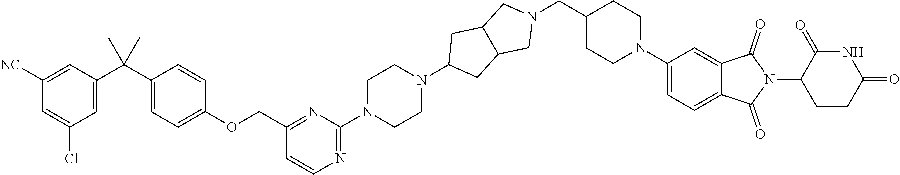 [SMILES: CC(C)(c1ccc(OCc2ccnc(N3CCN(C4CC5CN(CC6CCN(c7ccc8c(c7)C(=O)N(C7CCC(=O)NC7=O)C8=O)CC6)CC5C4)CC3)n2)cc1)c1cc(Cl)cc(C#N)c1]

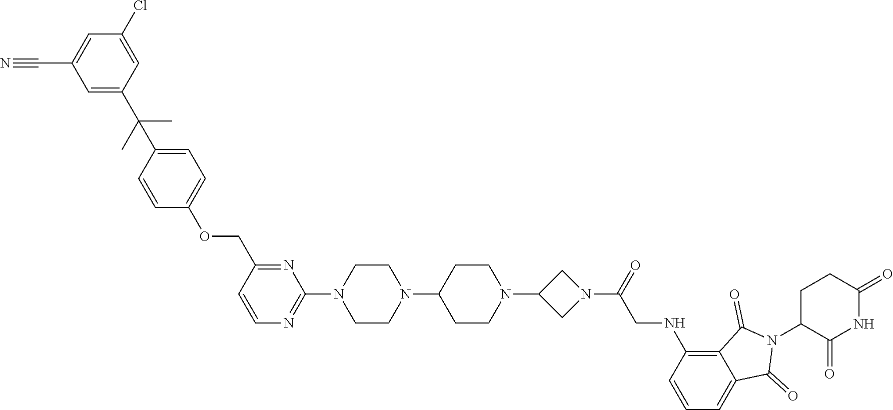 [SMILES: CC(C)(c1ccc(OCc2ccnc(N3CCN(C4CCN(C5CN(C(=O)CNc6cccc7c6C(=O)N(C6CCC(=O)NC6=O)C7=O)C5)CC4)CC3)n2)cc1)c1cc(Cl)cc(C#N)c1]

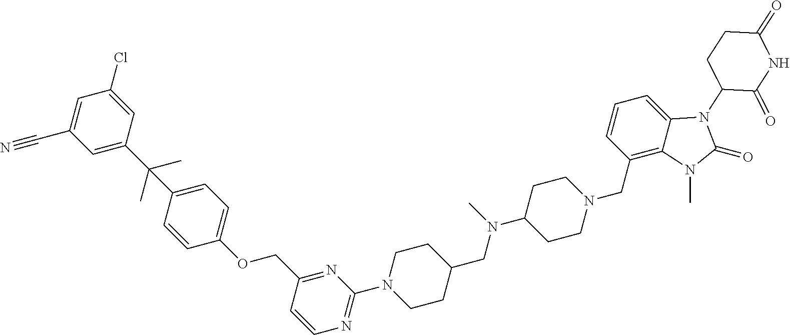 [SMILES: CN(CC1CCN(c2nccc(COc3ccc(C(C)(C)c4cc(Cl)cc(C#N)c4)cc3)n2)CC1)C1CCN(Cc2cccc3c2n(C)c(=O)n3C2CCC(=O)NC2=O)CC1]

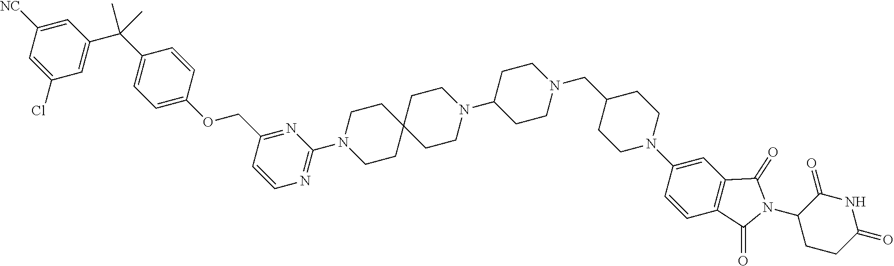 [SMILES: CC(C)(c1ccc(OCc2ccnc(N3CCC4(CC3)CCN(C3CCN(CC5CCN(c6ccc7c(c6)C(=O)N(C6CCC(=O)NC6=O)C7=O)CC5)CC3)CC4)n2)cc1)c1cc(Cl)cc(C#N)c1]

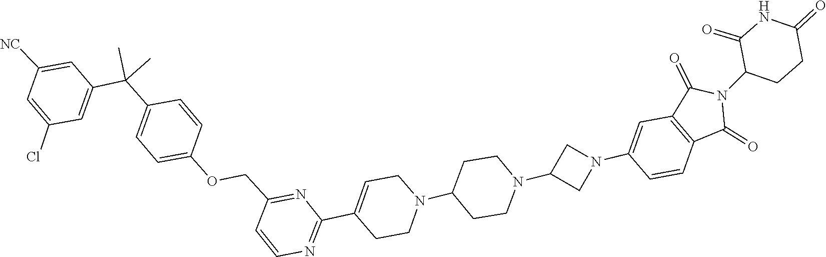 [SMILES: CC(C)(c1ccc(OCc2ccnc(C3=CCN(C4CCN(C5CN(c6ccc7c(c6)C(=O)N(C6CCC(=O)NC6=O)C7=O)C5)CC4)CC3)n2)cc1)c1cc(Cl)cc(C#N)c1]

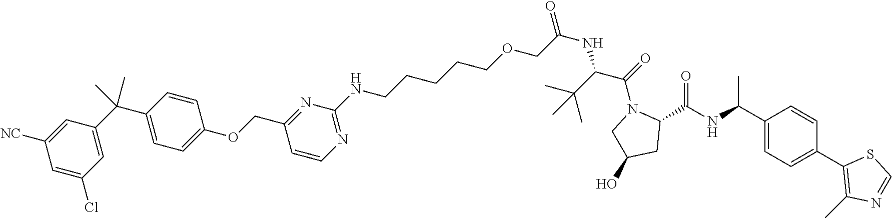 [SMILES: Cc1ncsc1-c1ccc([C@H](C)NC(=O)[C@@H]2C[C@@H](O)CN2C(=O)[C@@H](NC(=O)COCCCCCNc2nccc(COc3ccc(C(C)(C)c4cc(Cl)cc(C#N)c4)cc3)n2)C(C)(C)C)cc1]

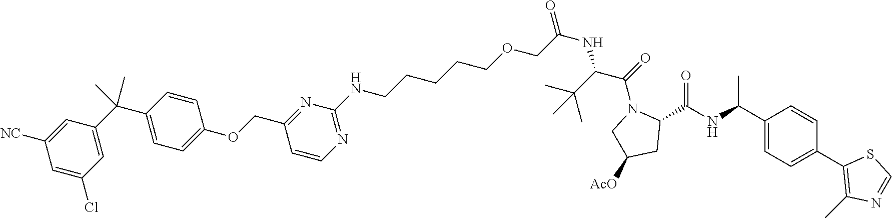 [SMILES: CC(=O)O[C@@H]1C[C@@H](C(=O)N[C@@H](C)c2ccc(-c3scnc3C)cc2)N(C(=O)[C@@H](NC(=O)COCCCCCNc2nccc(COc3ccc(C(C)(C)c4cc(Cl)cc(C#N)c4)cc3)n2)C(C)(C)C)C1]

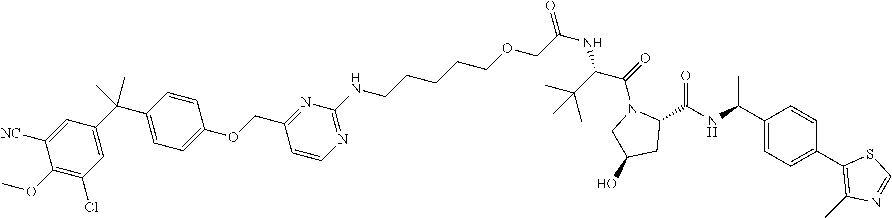 [SMILES: COc1c(Cl)cc(C(C)(C)c2ccc(OCc3ccnc(NCCCCCOCC(=O)N[C@H](C(=O)N4C[C@H](O)C[C@H]4C(=O)N[C@@H](C)c4ccc(-c5scnc5C)cc4)C(C)(C)C)n3)cc2)cc1C#N]

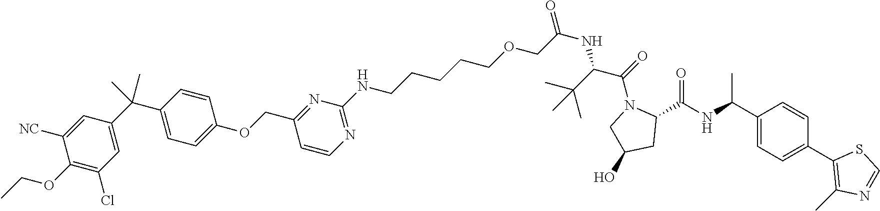 [SMILES: CCOc1c(Cl)cc(C(C)(C)c2ccc(OCc3ccnc(NCCCCCOCC(=O)N[C@H](C(=O)N4C[C@H](O)C[C@H]4C(=O)N[C@@H](C)c4ccc(-c5scnc5C)cc4)C(C)(C)C)n3)cc2)cc1C#N]

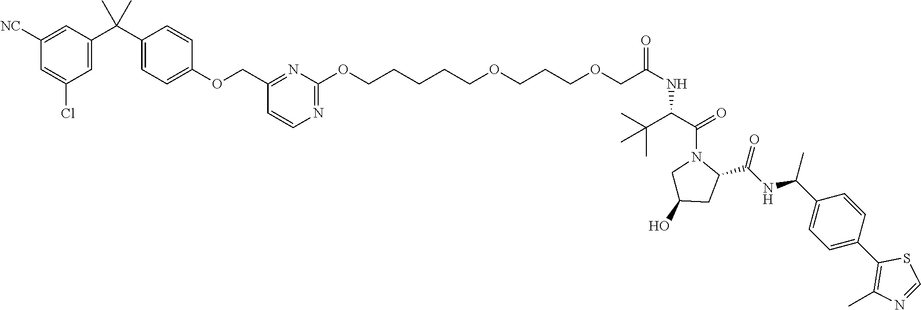 [SMILES: Cc1ncsc1-c1ccc([C@H](C)NC(=O)[C@@H]2C[C@@H](O)CN2C(=O)[C@@H](NC(=O)COCCCOCCCCCOc2nccc(COc3ccc(C(C)(C)c4cc(Cl)cc(C#N)c4)cc3)n2)C(C)(C)C)cc1]

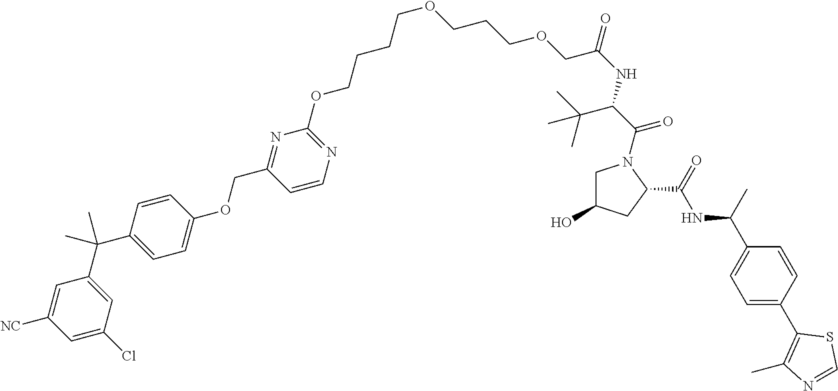 [SMILES: Cc1ncsc1-c1ccc([C@H](C)NC(=O)[C@@H]2C[C@@H](O)CN2C(=O)[C@@H](NC(=O)COCCCOCCCCOc2nccc(COc3ccc(C(C)(C)c4cc(Cl)cc(C#N)c4)cc3)n2)C(C)(C)C)cc1]

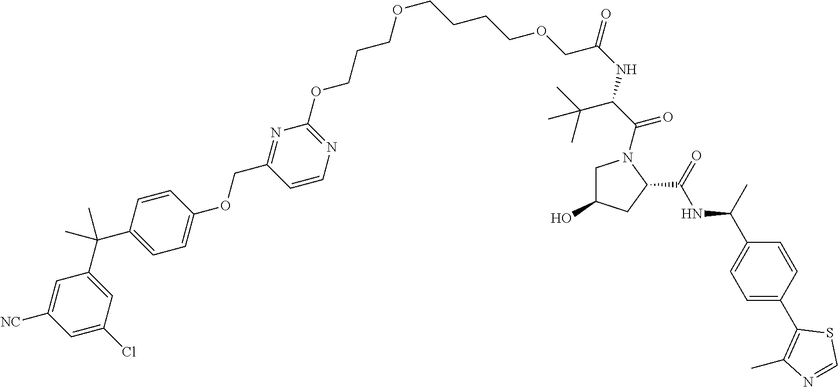 [SMILES: Cc1ncsc1-c1ccc([C@H](C)NC(=O)[C@@H]2C[C@@H](O)CN2C(=O)[C@@H](NC(=O)COCCCCOCCCOc2nccc(COc3ccc(C(C)(C)c4cc(Cl)cc(C#N)c4)cc3)n2)C(C)(C)C)cc1]

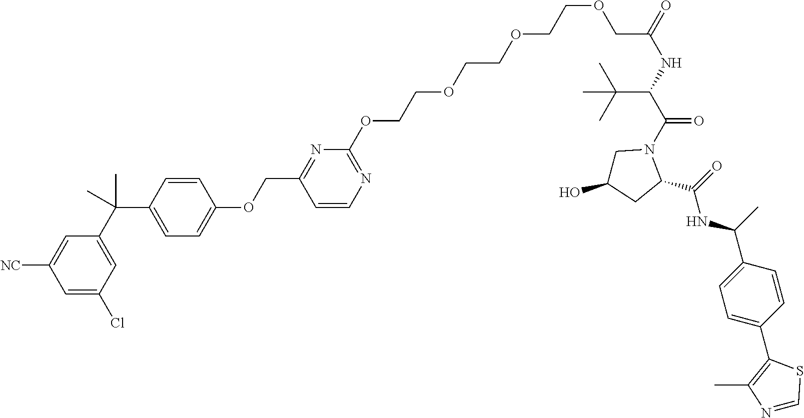 [SMILES: Cc1ncsc1-c1ccc([C@H](C)NC(=O)[C@@H]2C[C@@H](O)CN2C(=O)[C@@H](NC(=O)COCCOCCOCCOc2nccc(COc3ccc(C(C)(C)c4cc(Cl)cc(C#N)c4)cc3)n2)C(C)(C)C)cc1]

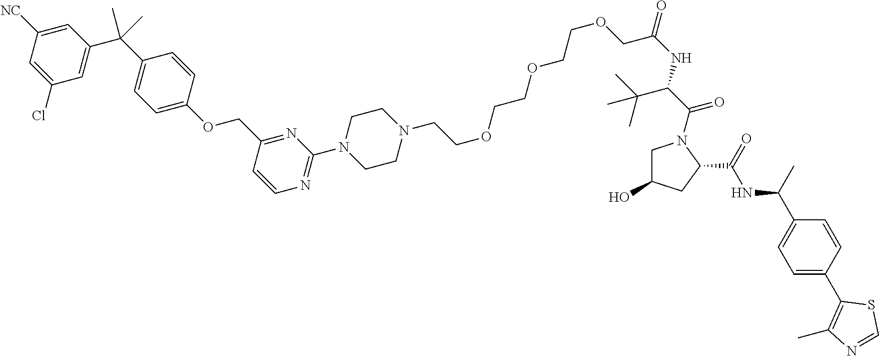 [SMILES: Cc1ncsc1-c1ccc([C@H](C)NC(=O)[C@@H]2C[C@@H](O)CN2C(=O)[C@@H](NC(=O)COCCOCCOCCN2CCN(c3nccc(COc4ccc(C(C)(C)c5cc(Cl)cc(C#N)c5)cc4)n3)CC2)C(C)(C)C)cc1]

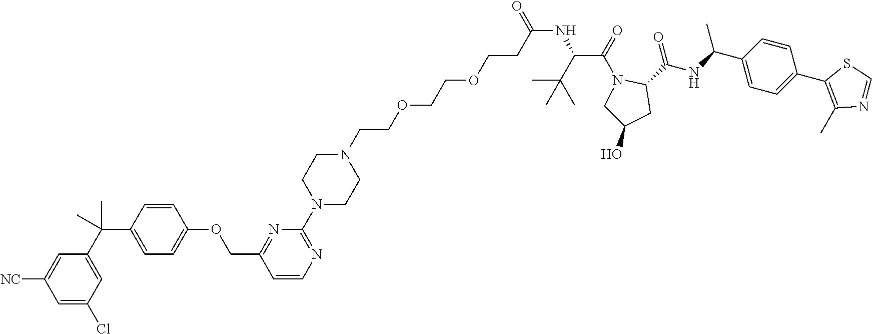 [SMILES: Cc1ncsc1-c1ccc([C@H](C)NC(=O)[C@@H]2C[C@@H](O)CN2C(=O)[C@@H](NC(=O)CCOCCOCCN2CCN(c3nccc(COc4ccc(C(C)(C)c5cc(Cl)cc(C#N)c5)cc4)n3)CC2)C(C)(C)C)cc1]

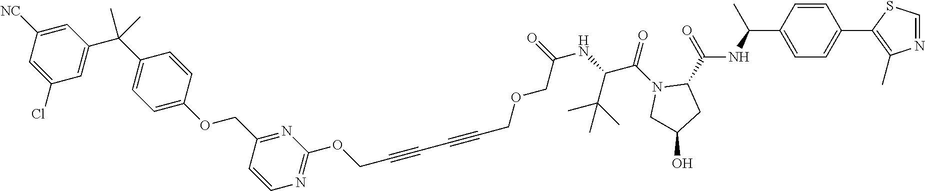 [SMILES: Cc1ncsc1-c1ccc([C@H](C)NC(=O)[C@@H]2C[C@@H](O)CN2C(=O)[C@@H](NC(=O)COCC#CC#CCOc2nccc(COc3ccc(C(C)(C)c4cc(Cl)cc(C#N)c4)cc3)n2)C(C)(C)C)cc1]

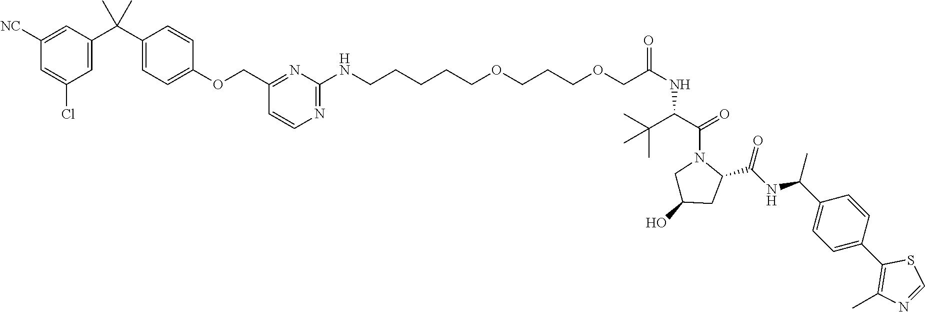 [SMILES: Cc1ncsc1-c1ccc([C@H](C)NC(=O)[C@@H]2C[C@@H](O)CN2C(=O)[C@@H](NC(=O)COCCCOCCCCCNc2nccc(COc3ccc(C(C)(C)c4cc(Cl)cc(C#N)c4)cc3)n2)C(C)(C)C)cc1]

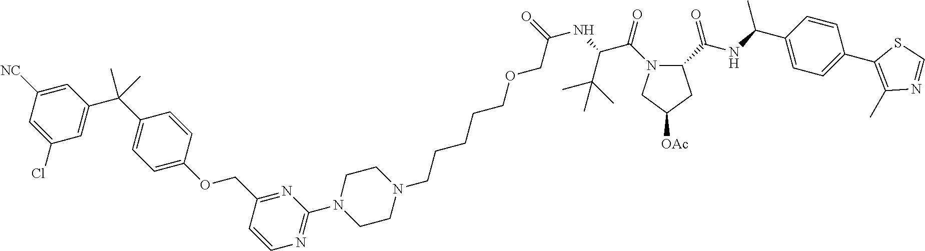 [SMILES: CC(=O)O[C@@H]1C[C@@H](C(=O)N[C@@H](C)c2ccc(-c3scnc3C)cc2)N(C(=O)[C@@H](NC(=O)COCCCCCN2CCN(c3nccc(COc4ccc(C(C)(C)c5cc(Cl)cc(C#N)c5)cc4)n3)CC2)C(C)(C)C)C1]

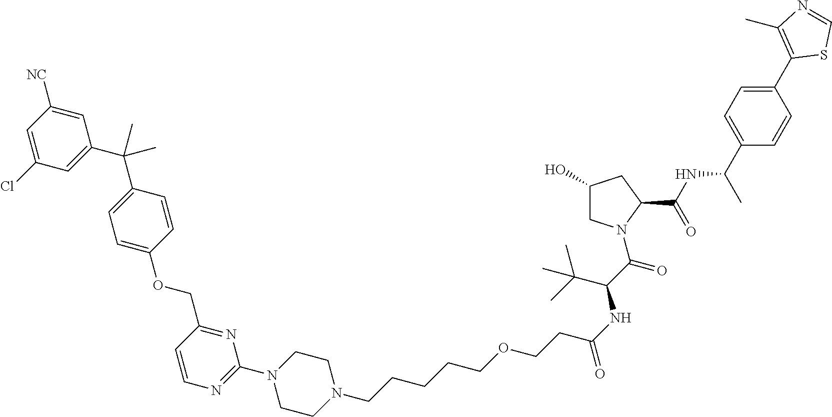 [SMILES: Cc1ncsc1-c1ccc([C@H](C)NC(=O)[C@@H]2C[C@@H](O)CN2C(=O)[C@@H](NC(=O)CCOCCCCCN2CCN(c3nccc(COc4ccc(C(C)(C)c5cc(Cl)cc(C#N)c5)cc4)n3)CC2)C(C)(C)C)cc1]

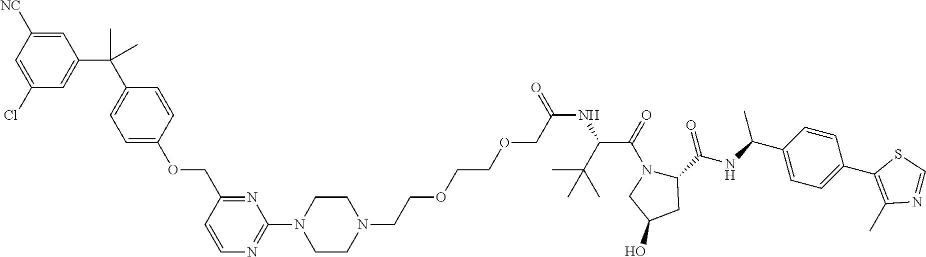 [SMILES: Cc1ncsc1-c1ccc([C@H](C)NC(=O)[C@@H]2C[C@@H](O)CN2C(=O)[C@@H](NC(=O)COCCOCCN2CCN(c3nccc(COc4ccc(C(C)(C)c5cc(Cl)cc(C#N)c5)cc4)n3)CC2)C(C)(C)C)cc1]

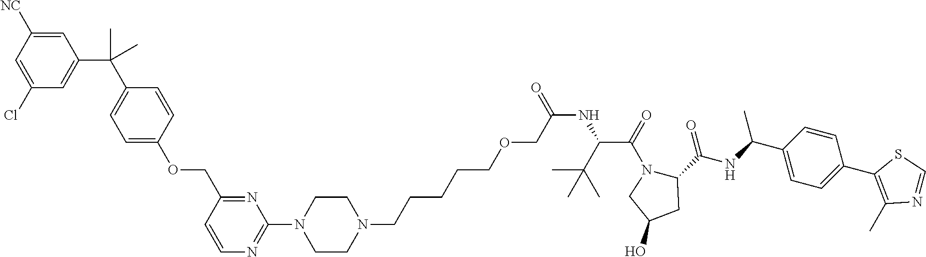 [SMILES: Cc1ncsc1-c1ccc([C@H](C)NC(=O)[C@@H]2C[C@@H](O)CN2C(=O)[C@@H](NC(=O)COCCCCCN2CCN(c3nccc(COc4ccc(C(C)(C)c5cc(Cl)cc(C#N)c5)cc4)n3)CC2)C(C)(C)C)cc1]

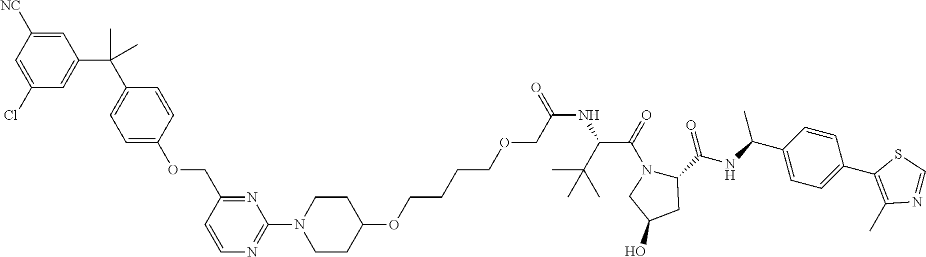 [SMILES: Cc1ncsc1-c1ccc([C@H](C)NC(=O)[C@@H]2C[C@@H](O)CN2C(=O)[C@@H](NC(=O)COCCCCOC2CCN(c3nccc(COc4ccc(C(C)(C)c5cc(Cl)cc(C#N)c5)cc4)n3)CC2)C(C)(C)C)cc1]

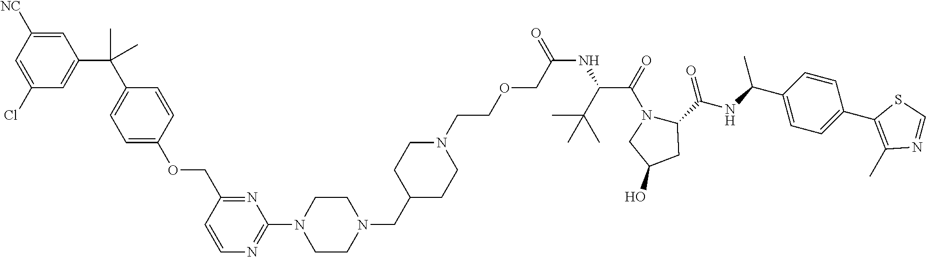 [SMILES: Cc1ncsc1-c1ccc([C@H](C)NC(=O)[C@@H]2C[C@@H](O)CN2C(=O)[C@@H](NC(=O)COCCN2CCC(CN3CCN(c4nccc(COc5ccc(C(C)(C)c6cc(Cl)cc(C#N)c6)cc5)n4)CC3)CC2)C(C)(C)C)cc1]

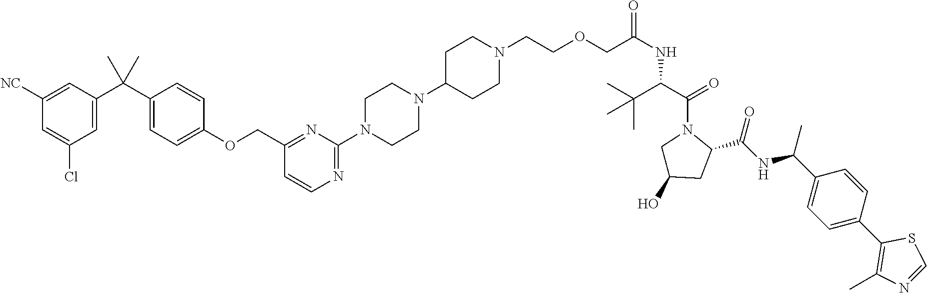 [SMILES: Cc1ncsc1-c1ccc([C@H](C)NC(=O)[C@@H]2C[C@@H](O)CN2C(=O)[C@@H](NC(=O)COCCN2CCC(N3CCN(c4nccc(COc5ccc(C(C)(C)c6cc(Cl)cc(C#N)c6)cc5)n4)CC3)CC2)C(C)(C)C)cc1]

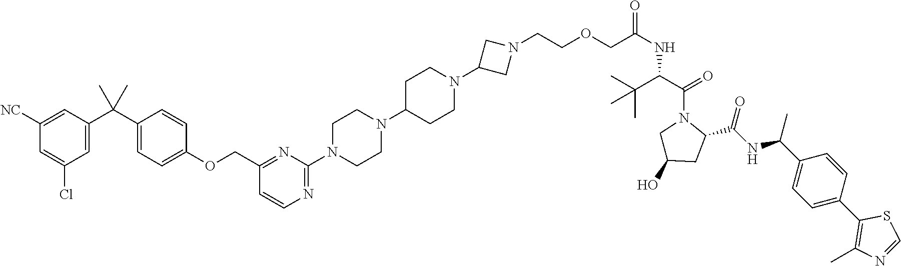 [SMILES: Cc1ncsc1-c1ccc([C@H](C)NC(=O)[C@@H]2C[C@@H](O)CN2C(=O)[C@@H](NC(=O)COCCN2CC(N3CCC(N4CCN(c5nccc(COc6ccc(C(C)(C)c7cc(Cl)cc(C#N)c7)cc6)n5)CC4)CC3)C2)C(C)(C)C)cc1]

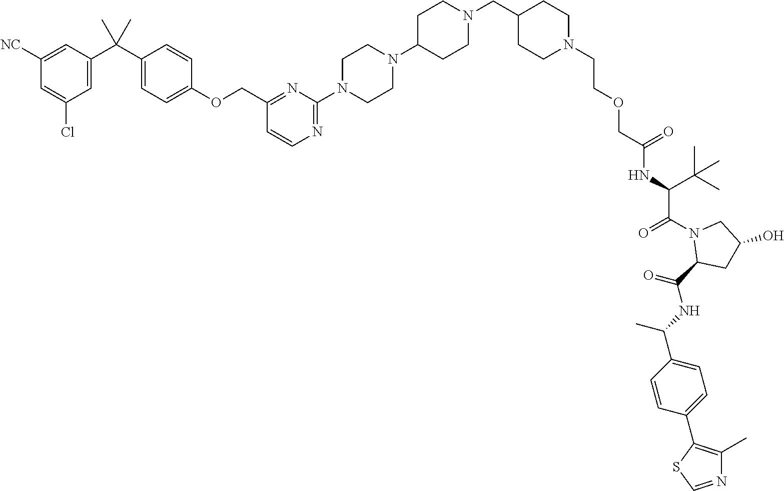 [SMILES: Cc1ncsc1-c1ccc([C@H](C)NC(=O)[C@@H]2C[C@@H](O)CN2C(=O)[C@@H](NC(=O)COCCN2CCC(CN3CCC(N4CCN(c5nccc(COc6ccc(C(C)(C)c7cc(Cl)cc(C#N)c7)cc6)n5)CC4)CC3)CC2)C(C)(C)C)cc1]

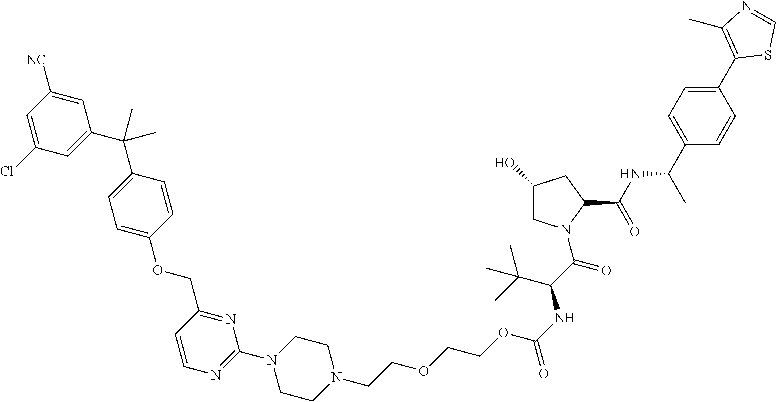

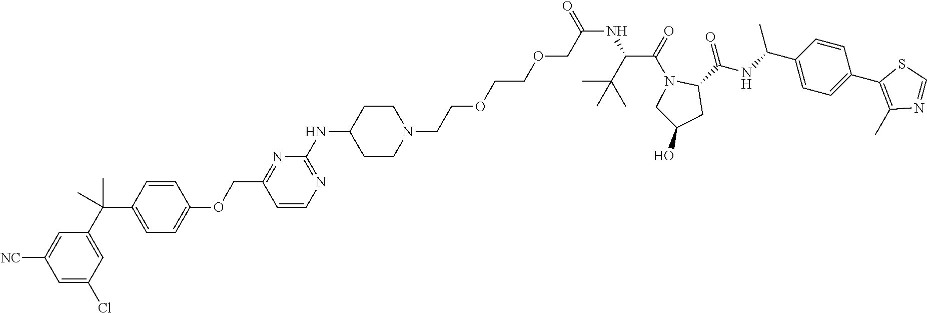 [SMILES: Cc1ncsc1-c1ccc([C@@H](C)NC(=O)[C@@H]2C[C@@H](O)CN2C(=O)[C@@H](NC(=O)COCCOCCN2CCC(Nc3nccc(COc4ccc(C(C)(C)c5cc(Cl)cc(C#N)c5)cc4)n3)CC2)C(C)(C)C)cc1]

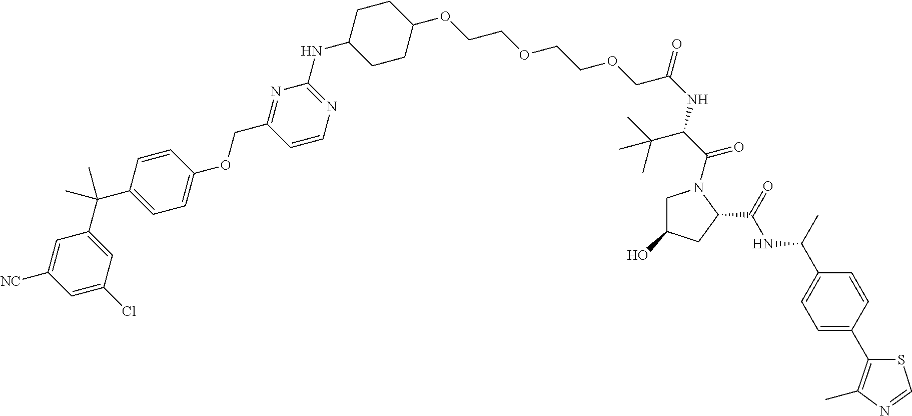 [SMILES: Cc1ncsc1-c1ccc([C@@H](C)NC(=O)[C@@H]2C[C@@H](O)CN2C(=O)[C@@H](NC(=O)COCCOCCOC2CCC(Nc3nccc(COc4ccc(C(C)(C)c5cc(Cl)cc(C#N)c5)cc4)n3)CC2)C(C)(C)C)cc1]

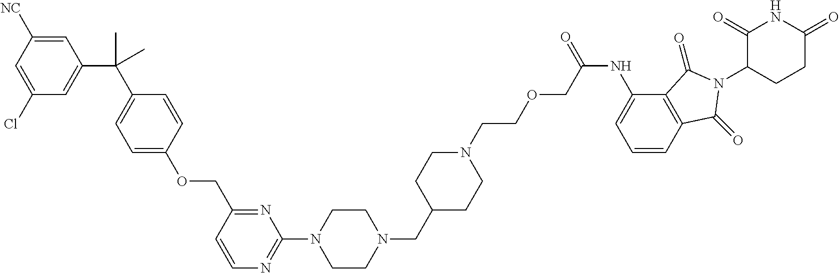 [SMILES: CC(C)(c1ccc(OCc2ccnc(N3CCN(CC4CCN(CCOCC(=O)Nc5cccc6c5C(=O)N(C5CCC(=O)NC5=O)C6=O)CC4)CC3)n2)cc1)c1cc(Cl)cc(C#N)c1]

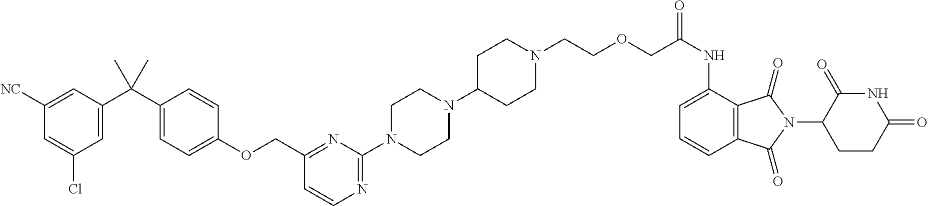 [SMILES: CC(C)(c1ccc(OCc2ccnc(N3CCN(C4CCN(CCOCC(=O)Nc5cccc6c5C(=O)N(C5CCC(=O)NC5=O)C6=O)CC4)CC3)n2)cc1)c1cc(Cl)cc(C#N)c1]

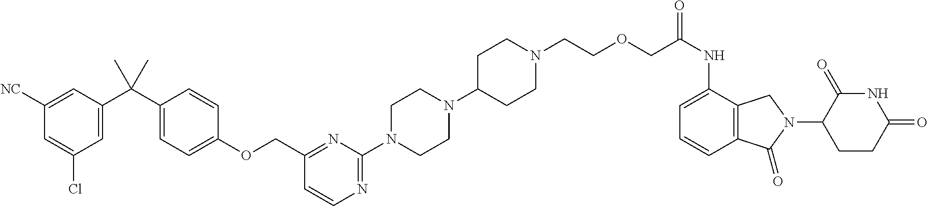 [SMILES: CC(C)(c1ccc(OCc2ccnc(N3CCN(C4CCN(CCOCC(=O)Nc5cccc6c5CN(C5CCC(=O)NC5=O)C6=O)CC4)CC3)n2)cc1)c1cc(Cl)cc(C#N)c1]

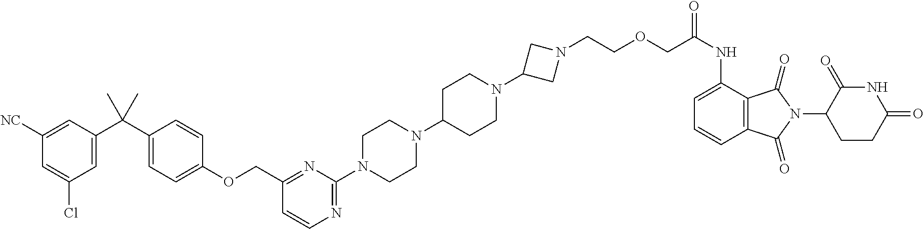 [SMILES: CC(C)(c1ccc(OCc2ccnc(N3CCN(C4CCN(C5CN(CCOCC(=O)Nc6cccc7c6C(=O)N(C6CCC(=O)NC6=O)C7=O)C5)CC4)CC3)n2)cc1)c1cc(Cl)cc(C#N)c1]

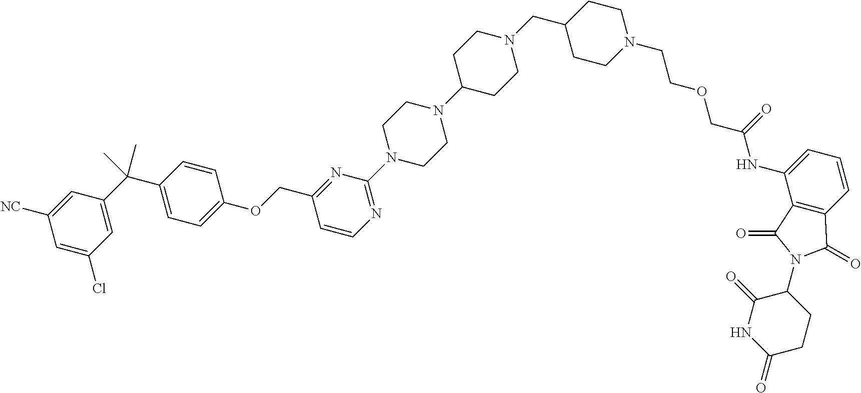 [SMILES: CC(C)(c1ccc(OCc2ccnc(N3CCN(C4CCN(CC5CCN(CCOCC(=O)Nc6cccc7c6C(=O)N(C6CCC(=O)NC6=O)C7=O)CC5)CC4)CC3)n2)cc1)c1cc(Cl)cc(C#N)c1]

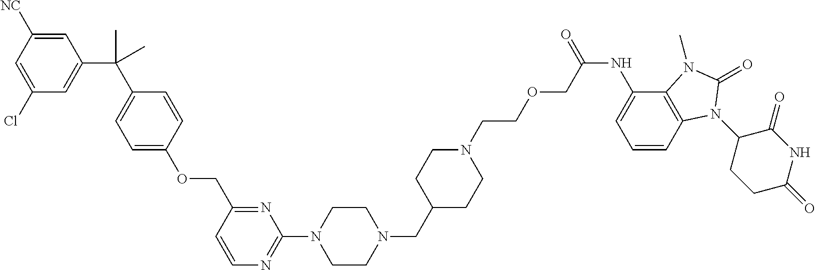 [SMILES: Cn1c(=O)n(C2CCC(=O)NC2=O)c2cccc(NC(=O)COCCN3CCC(CN4CCN(c5nccc(COc6ccc(C(C)(C)c7cc(Cl)cc(C#N)c7)cc6)n5)CC4)CC3)c21]

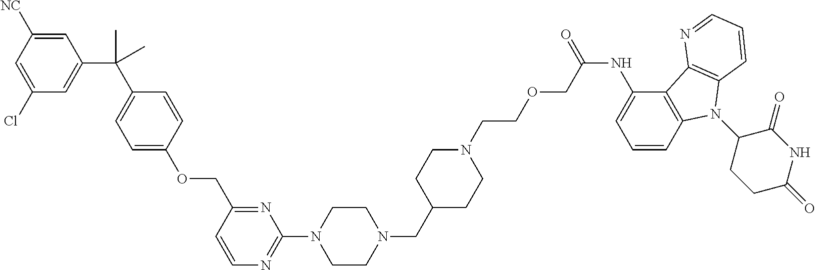 [SMILES: CC(C)(c1ccc(OCc2ccnc(N3CCN(CC4CCN(CCOCC(=O)Nc5cccc6c5c5ncccc5n6C5CCC(=O)NC5=O)CC4)CC3)n2)cc1)c1cc(Cl)cc(C#N)c1]

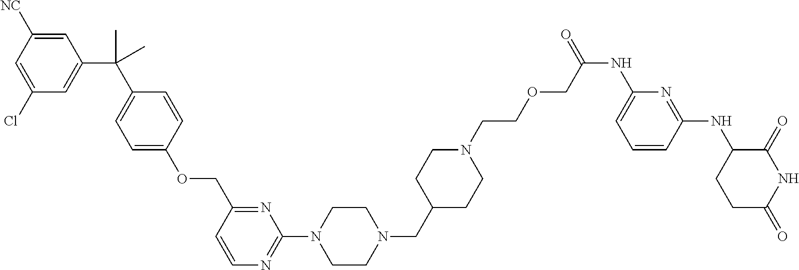 [SMILES: CC(C)(c1ccc(OCc2ccnc(N3CCN(CC4CCN(CCOCC(=O)Nc5cccc(NC6CCC(=O)NC6=O)n5)CC4)CC3)n2)cc1)c1cc(Cl)cc(C#N)c1]

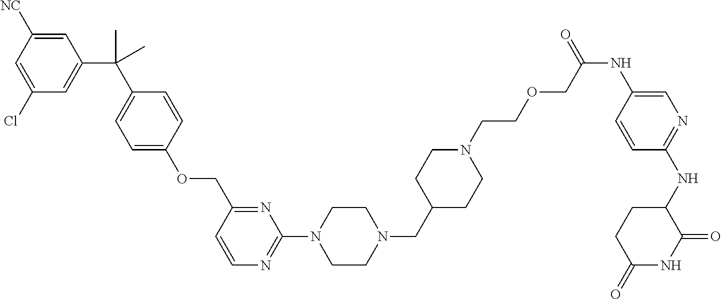 [SMILES: CC(C)(c1ccc(OCc2ccnc(N3CCN(CC4CCN(CCOCC(=O)Nc5ccc(NC6CCC(=O)NC6=O)nc5)CC4)CC3)n2)cc1)c1cc(Cl)cc(C#N)c1]

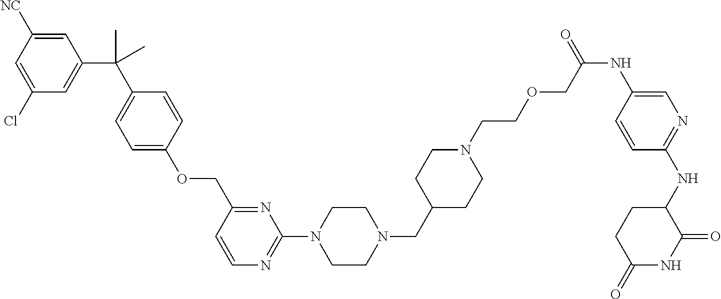 [SMILES: CC(C)(c1ccc(OCc2ccnc(N3CCN(CC4CCN(CCOCC(=O)Nc5ccc(NC6CCC(=O)NC6=O)nc5)CC4)CC3)n2)cc1)c1cc(Cl)cc(C#N)c1]

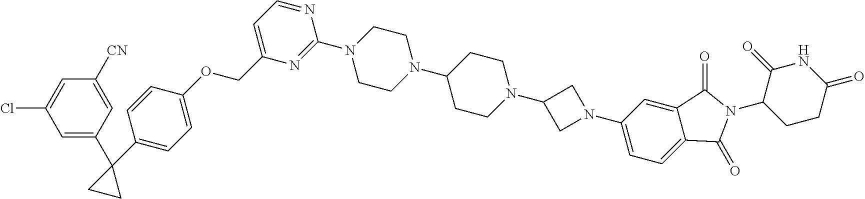 [SMILES: N#Cc1cc(Cl)cc(C2(c3ccc(OCc4ccnc(N5CCN(C6CCN(C7CN(c8ccc9c(c8)C(=O)N(C8CCC(=O)NC8=O)C9=O)C7)CC6)CC5)n4)cc3)CC2)c1]

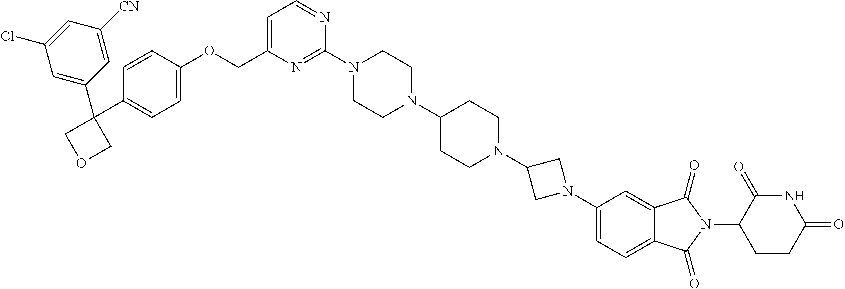 [SMILES: N#Cc1cc(Cl)cc(C2(c3ccc(OCc4ccnc(N5CCN(C6CCN(C7CN(c8ccc9c(c8)C(=O)N(C8CCC(=O)NC8=O)C9=O)C7)CC6)CC5)n4)cc3)COC2)c1]

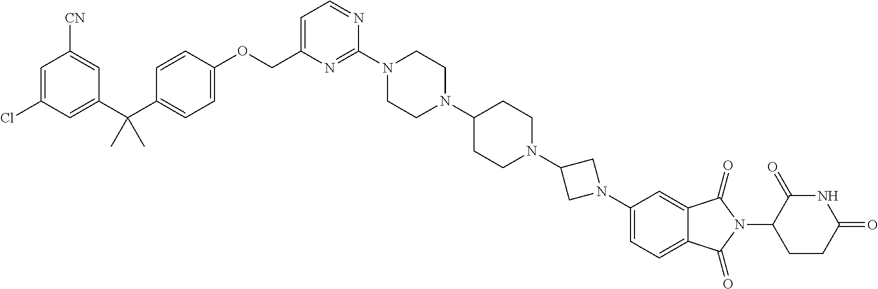 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)cc(C#N)c1]

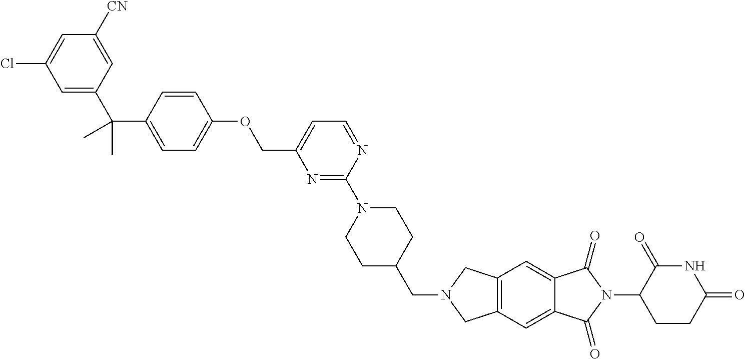 [SMILES: CC(C)(c1ccc(OCc2ccnc(N3CCC(CN4Cc5cc6c(cc5C4)C(=O)N(C4CCC(=O)NC4=O)C6=O)CC3)n2)cc1)c1cc(Cl)cc(C#N)c1]

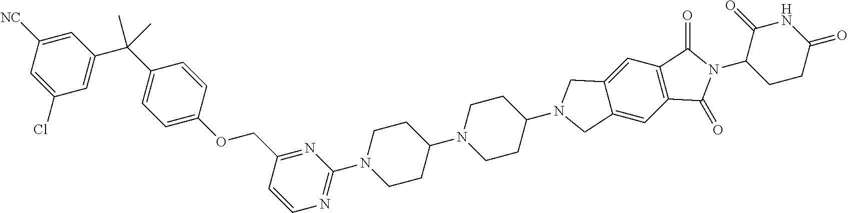 [SMILES: CC(C)(c1ccc(OCc2ccnc(N3CCC(N4CCC(N5Cc6cc7c(cc6C5)C(=O)N(C5CCC(=O)NC5=O)C7=O)CC4)CC3)n2)cc1)c1cc(Cl)cc(C#N)c1]

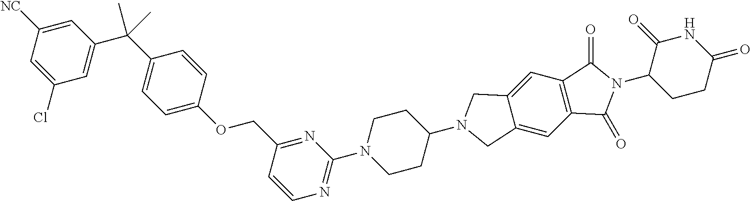 [SMILES: CC(C)(c1ccc(OCc2ccnc(N3CCC(N4Cc5cc6c(cc5C4)C(=O)N(C4CCC(=O)NC4=O)C6=O)CC3)n2)cc1)c1cc(Cl)cc(C#N)c1]

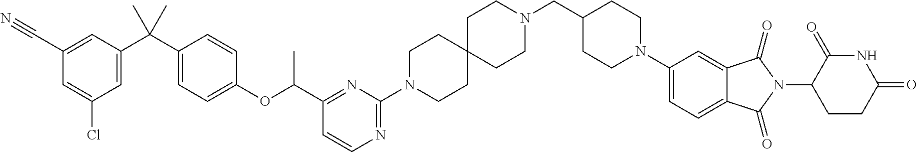 [SMILES: CC(Oc1ccc(C(C)(C)c2cc(Cl)cc(C#N)c2)cc1)c1ccnc(N2CCC3(CCN(CC4CCN(c5ccc6c(c5)C(=O)N(C5CCC(=O)NC5=O)C6=O)CC4)CC3)CC2)n1]

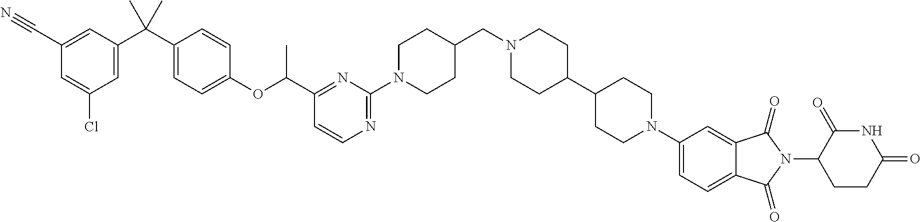 [SMILES: CC(Oc1ccc(C(C)(C)c2cc(Cl)cc(C#N)c2)cc1)c1ccnc(N2CCC(CN3CCC(C4CCN(c5ccc6c(c5)C(=O)N(C5CCC(=O)NC5=O)C6=O)CC4)CC3)CC2)n1]

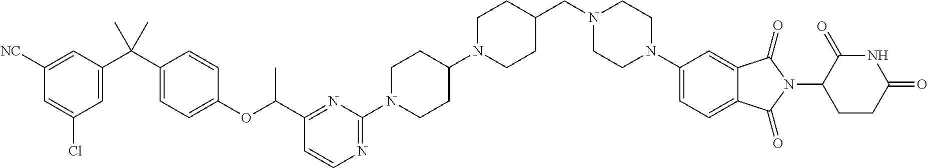 [SMILES: CC(Oc1ccc(C(C)(C)c2cc(Cl)cc(C#N)c2)cc1)c1ccnc(N2CCC(N3CCC(CN4CCN(c5ccc6c(c5)C(=O)N(C5CCC(=O)NC5=O)C6=O)CC4)CC3)CC2)n1]

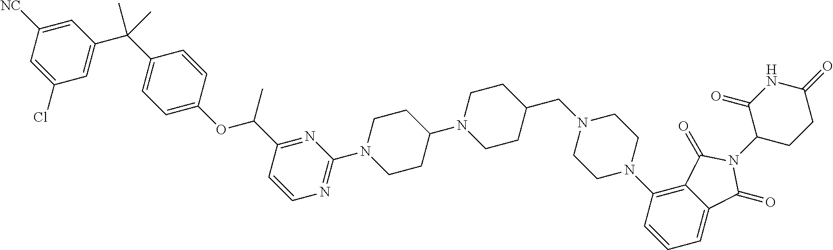 [SMILES: CC(Oc1ccc(C(C)(C)c2cc(Cl)cc(C#N)c2)cc1)c1ccnc(N2CCC(N3CCC(CN4CCN(c5cccc6c5C(=O)N(C5CCC(=O)NC5=O)C6=O)CC4)CC3)CC2)n1]

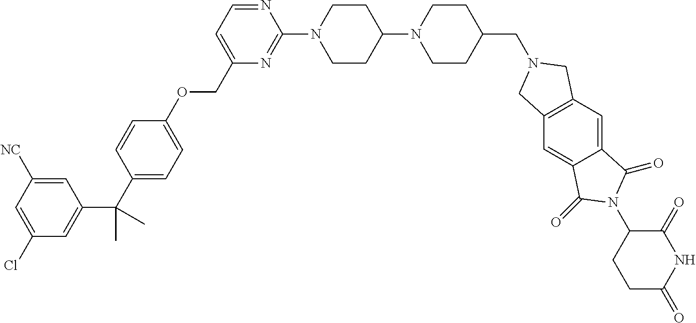 [SMILES: CC(C)(c1ccc(OCc2ccnc(N3CCC(N4CCC(CN5Cc6cc7c(cc6C5)C(=O)N(C5CCC(=O)NC5=O)C7=O)CC4)CC3)n2)cc1)c1cc(Cl)cc(C#N)c1]

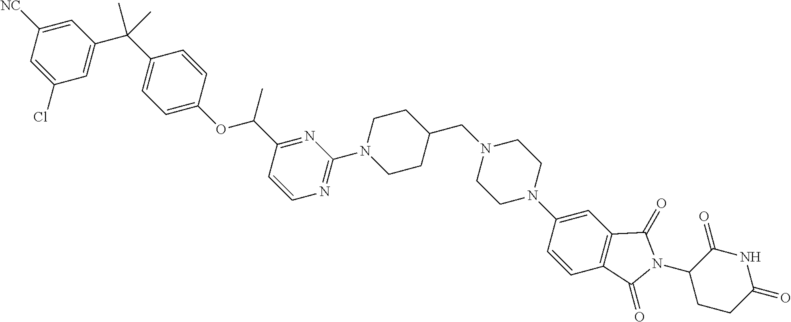 [SMILES: CC(Oc1ccc(C(C)(C)c2cc(Cl)cc(C#N)c2)cc1)c1ccnc(N2CCC(CN3CCN(c4ccc5c(c4)C(=O)N(C4CCC(=O)NC4=O)C5=O)CC3)CC2)n1]

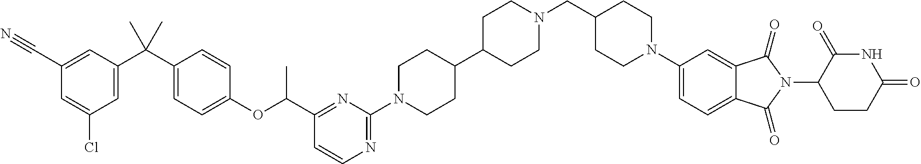 [SMILES: CC(Oc1ccc(C(C)(C)c2cc(Cl)cc(C#N)c2)cc1)c1ccnc(N2CCC(C3CCN(CC4CCN(c5ccc6c(c5)C(=O)N(C5CCC(=O)NC5=O)C6=O)CC4)CC3)CC2)n1]

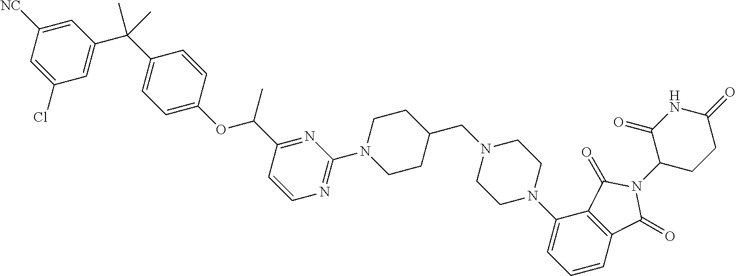 [SMILES: CC(Oc1ccc(C(C)(C)c2cc(Cl)cc(C#N)c2)cc1)c1ccnc(N2CCC(CN3CCN(c4cccc5c4C(=O)N(C4CCC(=O)NC4=O)C5=O)CC3)CC2)n1]

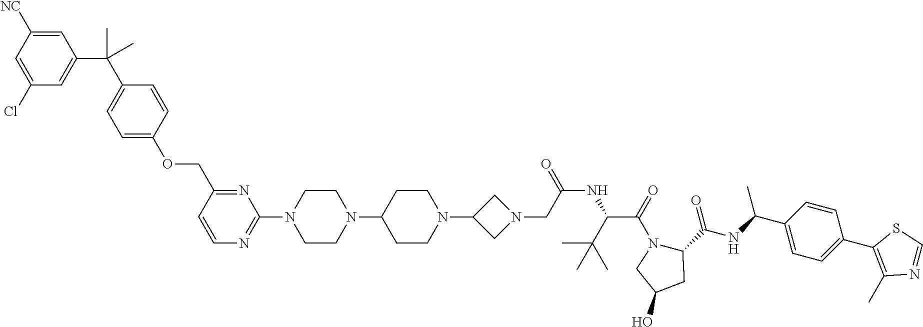 [SMILES: Cc1ncsc1-c1ccc([C@H](C)NC(=O)[C@@H]2C[C@@H](O)CN2C(=O)[C@@H](NC(=O)CN2CC(N3CCC(N4CCN(c5nccc(COc6ccc(C(C)(C)c7cc(Cl)cc(C#N)c7)cc6)n5)CC4)CC3)C2)C(C)(C)C)cc1]

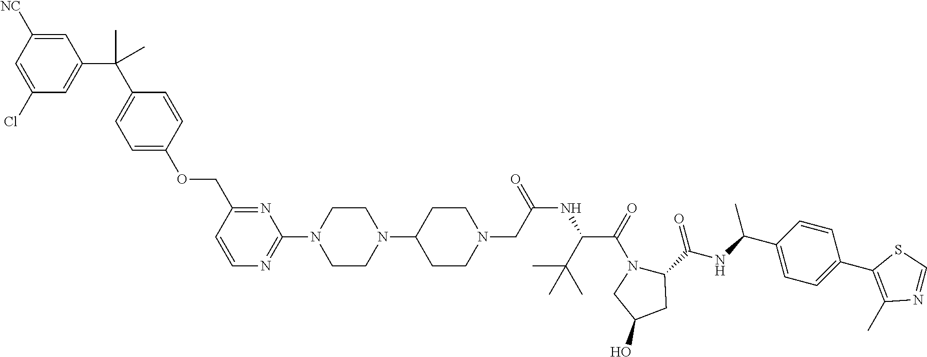 [SMILES: Cc1ncsc1-c1ccc([C@H](C)NC(=O)[C@@H]2C[C@@H](O)CN2C(=O)[C@@H](NC(=O)CN2CCC(N3CCN(c4nccc(COc5ccc(C(C)(C)c6cc(Cl)cc(C#N)c6)cc5)n4)CC3)CC2)C(C)(C)C)cc1]

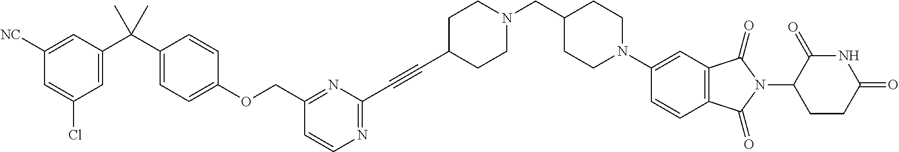 [SMILES: CC(C)(c1ccc(OCc2ccnc(C#CC3CCN(CC4CCN(c5ccc6c(c5)C(=O)N(C5CCC(=O)NC5=O)C6=O)CC4)CC3)n2)cc1)c1cc(Cl)cc(C#N)c1]

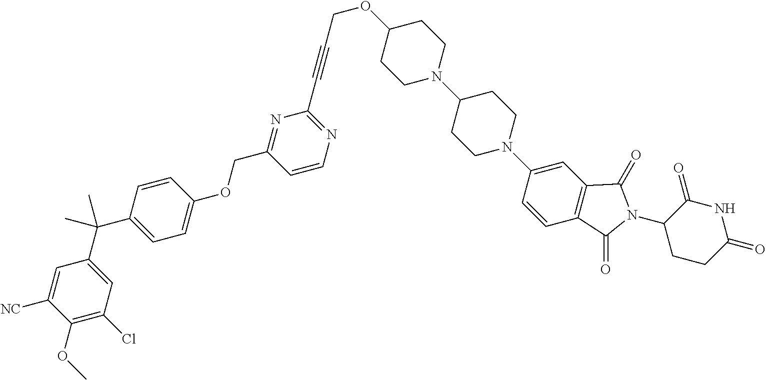 [SMILES: COc1c(Cl)cc(C(C)(C)c2ccc(OCc3ccnc(C#CCOC4CCN(C5CCN(c6ccc7c(c6)C(=O)N(C6CCC(=O)NC6=O)C7=O)CC5)CC4)n3)cc2)cc1C#N]

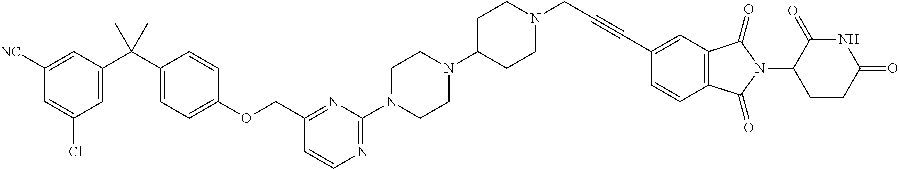 [SMILES: CC(C)(c1ccc(OCc2ccnc(N3CCN(C4CCN(CC#Cc5ccc6c(c5)C(=O)N(C5CCC(=O)NC5=O)C6=O)CC4)CC3)n2)cc1)c1cc(Cl)cc(C#N)c1]

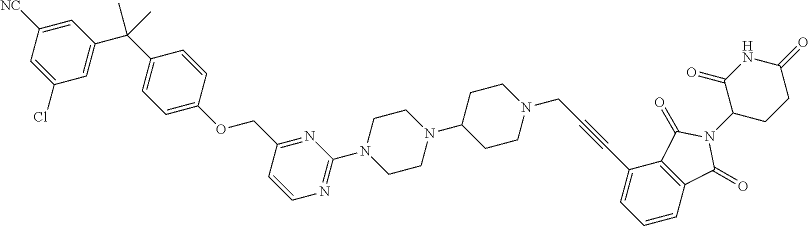 [SMILES: CC(C)(c1ccc(OCc2ccnc(N3CCN(C4CCN(CC#Cc5cccc6c5C(=O)N(C5CCC(=O)NC5=O)C6=O)CC4)CC3)n2)cc1)c1cc(Cl)cc(C#N)c1]

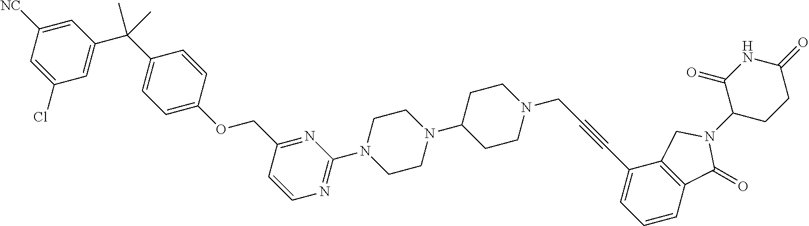 [SMILES: CC(C)(c1ccc(OCc2ccnc(N3CCN(C4CCN(CC#Cc5cccc6c5CN(C5CCC(=O)NC5=O)C6=O)CC4)CC3)n2)cc1)c1cc(Cl)cc(C#N)c1]

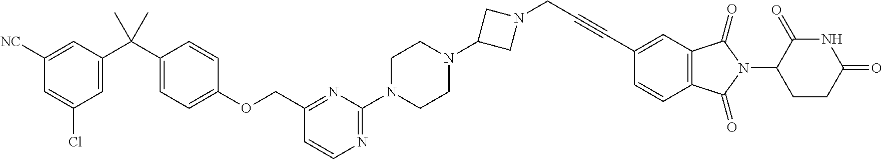 [SMILES: CC(C)(c1ccc(OCc2ccnc(N3CCN(C4CN(CC#Cc5ccc6c(c5)C(=O)N(C5CCC(=O)NC5=O)C6=O)C4)CC3)n2)cc1)c1cc(Cl)cc(C#N)c1]

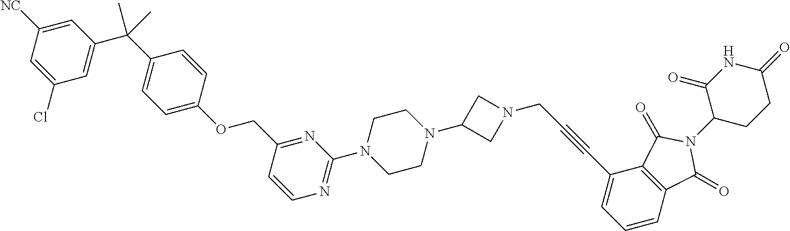 [SMILES: CC(C)(c1ccc(OCc2ccnc(N3CCN(C4CN(CC#Cc5cccc6c5C(=O)N(C5CCC(=O)NC5=O)C6=O)C4)CC3)n2)cc1)c1cc(Cl)cc(C#N)c1]

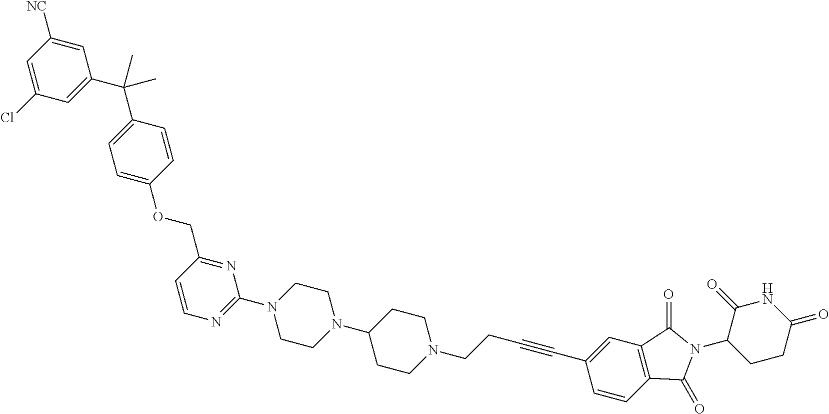 [SMILES: CC(C)(c1ccc(OCc2ccnc(N3CCN(C4CCN(CCC#Cc5ccc6c(c5)C(=O)N(C5CCC(=O)NC5=O)C6=O)CC4)CC3)n2)cc1)c1cc(Cl)cc(C#N)c1]

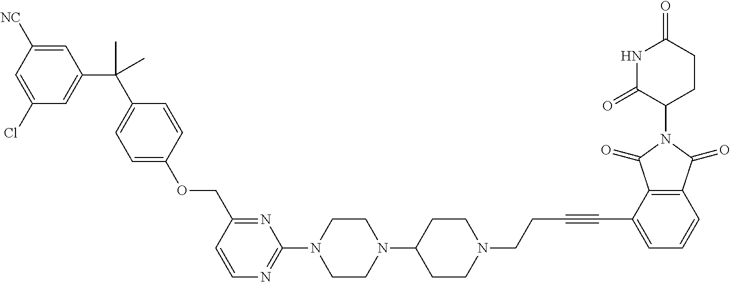 [SMILES: CC(C)(c1ccc(OCc2ccnc(N3CCN(C4CCN(CCC#Cc5cccc6c5C(=O)N(C5CCC(=O)NC5=O)C6=O)CC4)CC3)n2)cc1)c1cc(Cl)cc(C#N)c1]

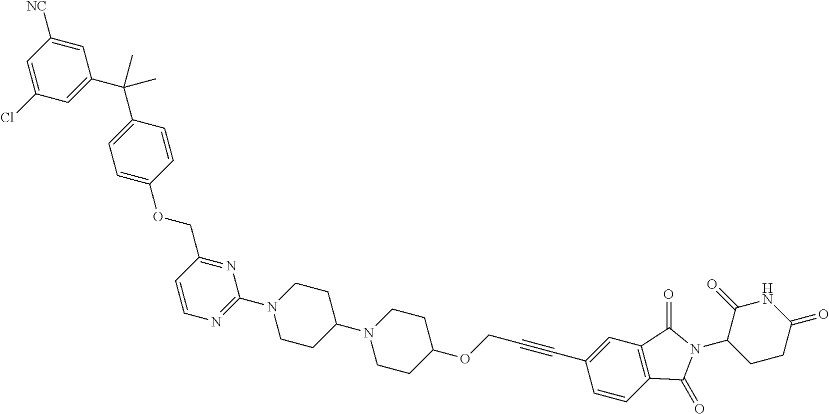 [SMILES: CC(C)(c1ccc(OCc2ccnc(N3CCC(N4CCC(OCC#Cc5ccc6c(c5)C(=O)N(C5CCC(=O)NC5=O)C6=O)CC4)CC3)n2)cc1)c1cc(Cl)cc(C#N)c1]

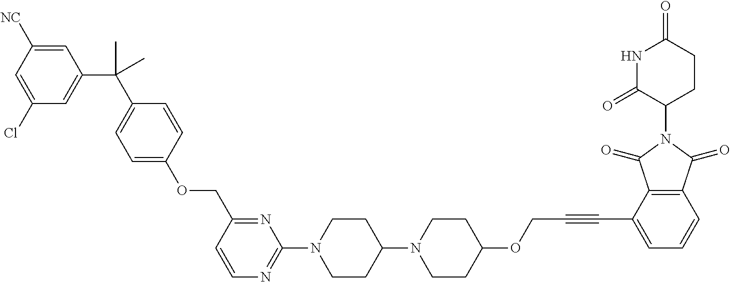 [SMILES: CC(C)(c1ccc(OCc2ccnc(N3CCC(N4CCC(OCC#Cc5cccc6c5C(=O)N(C5CCC(=O)NC5=O)C6=O)CC4)CC3)n2)cc1)c1cc(Cl)cc(C#N)c1]

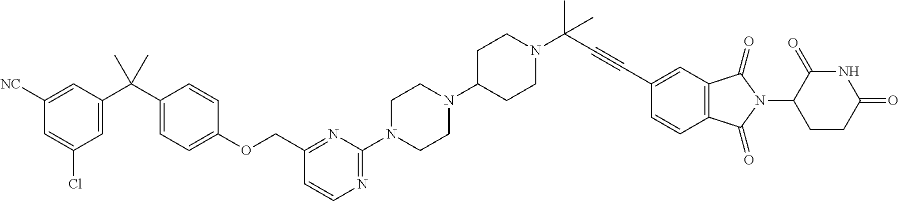 [SMILES: CC(C)(c1ccc(OCc2ccnc(N3CCN(C4CCN(C(C)(C)C#Cc5ccc6c(c5)C(=O)N(C5CCC(=O)NC5=O)C6=O)CC4)CC3)n2)cc1)c1cc(Cl)cc(C#N)c1]

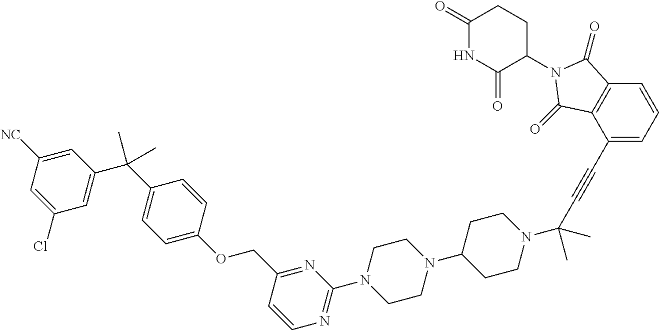 [SMILES: CC(C)(c1ccc(OCc2ccnc(N3CCN(C4CCN(C(C)(C)C#Cc5cccc6c5C(=O)N(C5CCC(=O)NC5=O)C6=O)CC4)CC3)n2)cc1)c1cc(Cl)cc(C#N)c1]

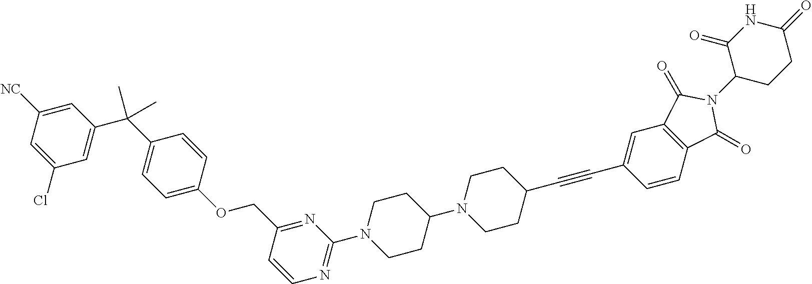 [SMILES: CC(C)(c1ccc(OCc2ccnc(N3CCC(N4CCC(C#Cc5ccc6c(c5)C(=O)N(C5CCC(=O)NC5=O)C6=O)CC4)CC3)n2)cc1)c1cc(Cl)cc(C#N)c1]

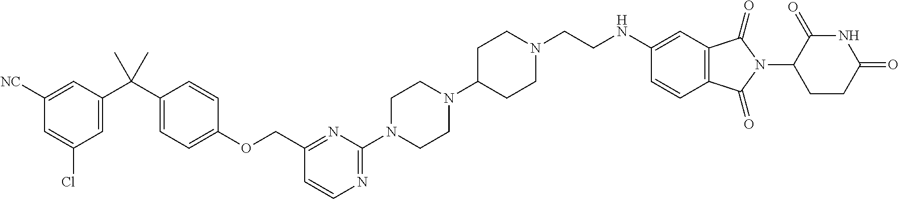 [SMILES: CC(C)(c1ccc(OCc2ccnc(N3CCN(C4CCN(CCNc5ccc6c(c5)C(=O)N(C5CCC(=O)NC5=O)C6=O)CC4)CC3)n2)cc1)c1cc(Cl)cc(C#N)c1]

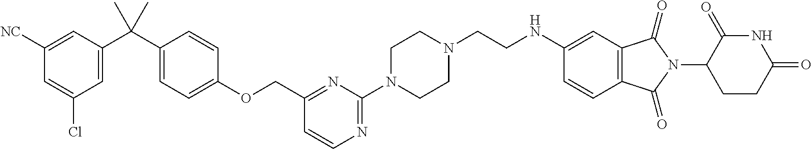 [SMILES: CC(C)(c1ccc(OCc2ccnc(N3CCN(CCNc4ccc5c(c4)C(=O)N(C4CCC(=O)NC4=O)C5=O)CC3)n2)cc1)c1cc(Cl)cc(C#N)c1]

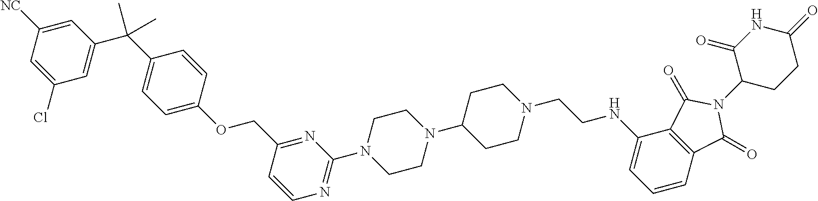 [SMILES: CC(C)(c1ccc(OCc2ccnc(N3CCN(C4CCN(CCNc5cccc6c5C(=O)N(C5CCC(=O)NC5=O)C6=O)CC4)CC3)n2)cc1)c1cc(Cl)cc(C#N)c1]

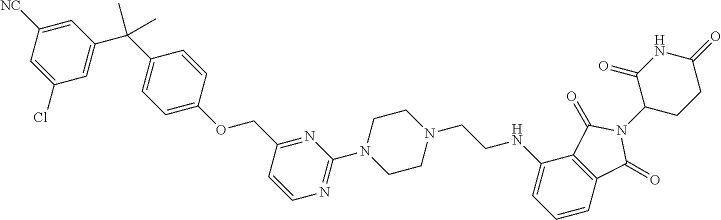 [SMILES: CC(C)(c1ccc(OCc2ccnc(N3CCN(CCNc4cccc5c4C(=O)N(C4CCC(=O)NC4=O)C5=O)CC3)n2)cc1)c1cc(Cl)cc(C#N)c1]

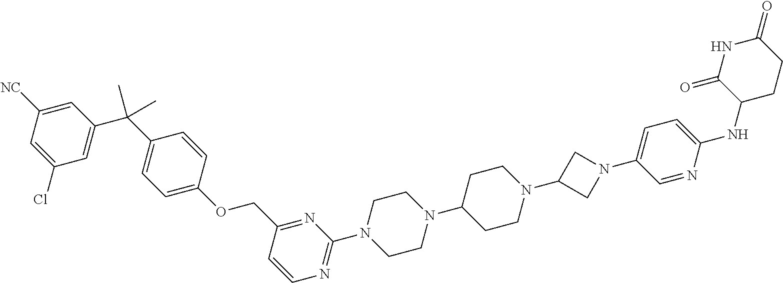 [SMILES: CC(C)(c1ccc(OCc2ccnc(N3CCN(C4CCN(C5CN(c6ccc(NC7CCC(=O)NC7=O)nc6)C5)CC4)CC3)n2)cc1)c1cc(Cl)cc(C#N)c1]

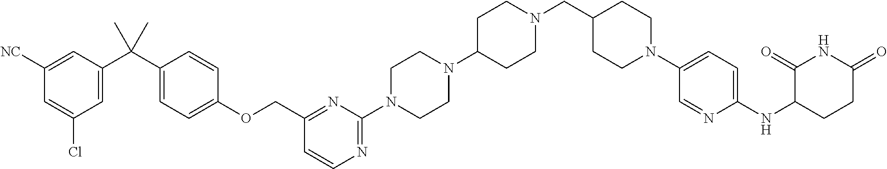 [SMILES: CC(C)(c1ccc(OCc2ccnc(N3CCN(C4CCN(CC5CCN(c6ccc(NC7CCC(=O)NC7=O)nc6)CC5)CC4)CC3)n2)cc1)c1cc(Cl)cc(C#N)c1]

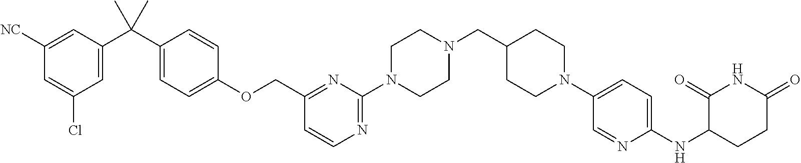 [SMILES: CC(C)(c1ccc(OCc2ccnc(N3CCN(CC4CCN(c5ccc(NC6CCC(=O)NC6=O)nc5)CC4)CC3)n2)cc1)c1cc(Cl)cc(C#N)c1]

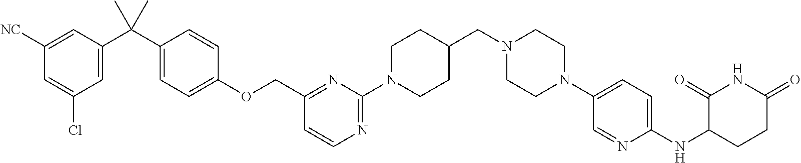 [SMILES: CC(C)(c1ccc(OCc2ccnc(N3CCC(CN4CCN(c5ccc(NC6CCC(=O)NC6=O)nc5)CC4)CC3)n2)cc1)c1cc(Cl)cc(C#N)c1]

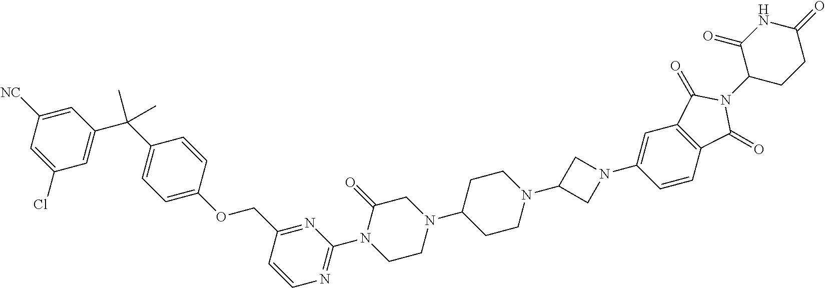 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3=O)n2)cc1)c1cc(Cl)cc(C#N)c1]

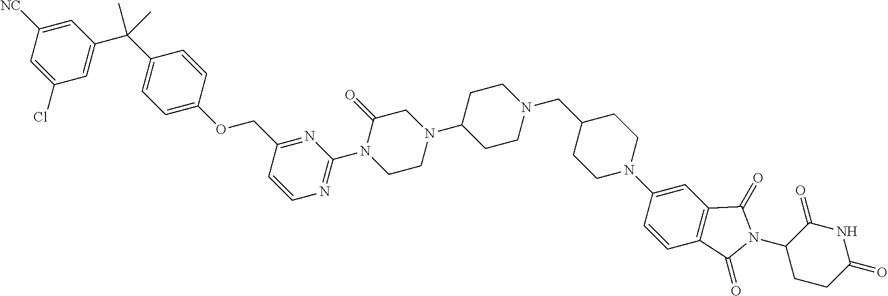 [SMILES: CC(C)(c1ccc(OCc2ccnc(N3CCN(C4CCN(CC5CCN(c6ccc7c(c6)C(=O)N(C6CCC(=O)NC6=O)C7=O)CC5)CC4)CC3=O)n2)cc1)c1cc(Cl)cc(C#N)c1]

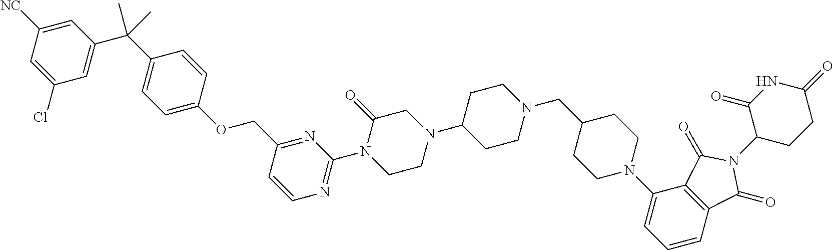 [SMILES: CC(C)(c1ccc(OCc2ccnc(N3CCN(C4CCN(CC5CCN(c6cccc7c6C(=O)N(C6CCC(=O)NC6=O)C7=O)CC5)CC4)CC3=O)n2)cc1)c1cc(Cl)cc(C#N)c1]

TABLE L-1

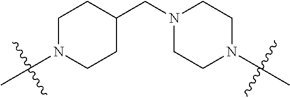 [SMILES: *N1CCC(CN2CCN(*)CC2)CC1]

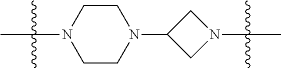 [SMILES: *N1CCN(C2CN(*)C2)CC1]

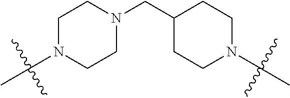 [SMILES: *N1CCC(CN2CCN(*)CC2)CC1]

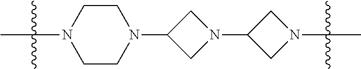 [SMILES: *N1CCN(C2CN(C3CN(*)C3)C2)CC1]

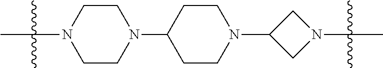 [SMILES: *N1CCN(C2CCN(C3CN(*)C3)CC2)CC1]

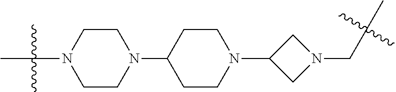 [SMILES: *CN1CC(N2CCC(N3CCN(*)CC3)CC2)C1]

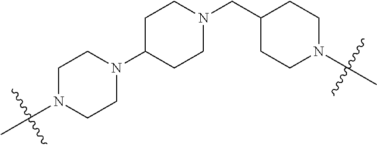 [SMILES: *N1CCC(CN2CCC(N3CCN(*)CC3)CC2)CC1]

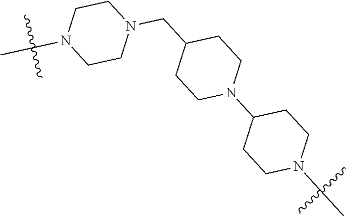 [SMILES: *N1CCC(N2CCC(CN3CCN(*)CC3)CC2)CC1]

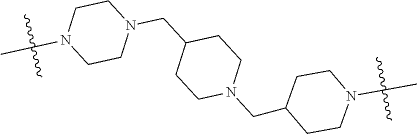 [SMILES: *N1CCC(CN2CCC(CN3CCN(*)CC3)CC2)CC1]

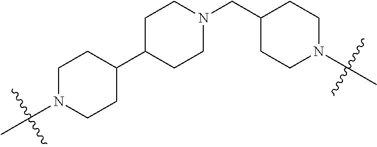 [SMILES: *N1CCC(CN2CCC(C3CCN(*)CC3)CC2)CC1]

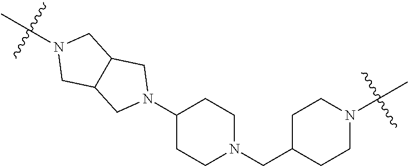 [SMILES: *N1CCC(CN2CCC(N3CC4CN(*)CC4C3)CC2)CC1]

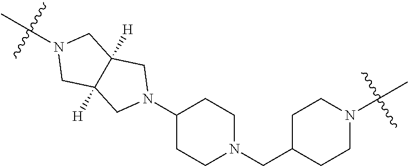 [SMILES: *N1CCC(CN2CCC(N3C[C@H]4CN(*)C[C@H]4C3)CC2)CC1]

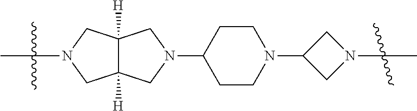 [SMILES: *N1CC(N2CCC(N3C[C@H]4CN(*)C[C@H]4C3)CC2)C1]

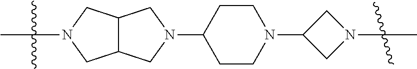 [SMILES: *N1CC2CN(C3CCN(C4CN(*)C4)CC3)CC2C1]

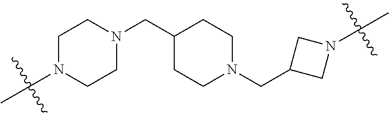 [SMILES: *N1CCN(CC2CCN(CC3CN(*)C3)CC2)CC1]

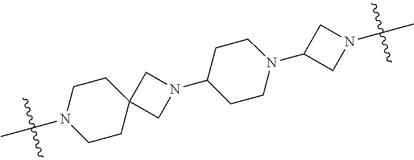 [SMILES: *N1CCC2(CC1)CN(C1CCN(C3CN(*)C3)CC1)C2]

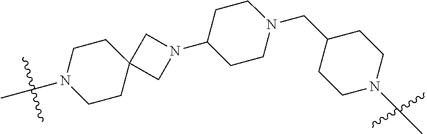 [SMILES: *N1CCC(CN2CCC(N3CC4(CCN(*)CC4)C3)CC2)CC1]

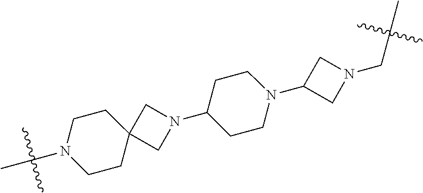 [SMILES: *CN1CC(N2CCC(N3CC4(CCN(*)CC4)C3)CC2)C1]

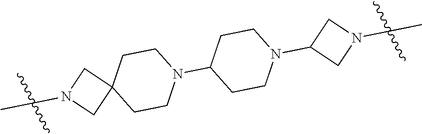 [SMILES: *N1CC(N2CCC(N3CCC4(CC3)CN(*)C4)CC2)C1]

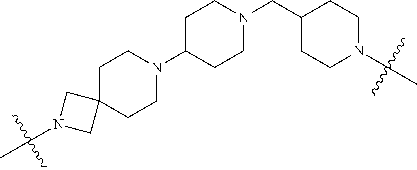 [SMILES: *N1CCC(CN2CCC(N3CCC4(CC3)CN(*)C4)CC2)CC1]

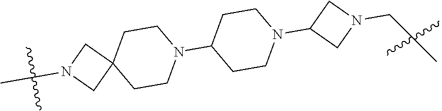 [SMILES: *CN1CC(N2CCC(N3CCC4(CC3)CN(*)C4)CC2)C1]

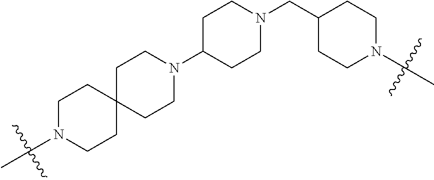 [SMILES: *N1CCC(CN2CCC(N3CCC4(CCN(*)CC4)CC3)CC2)CC1]

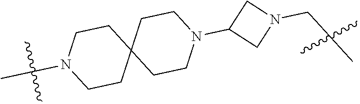 [SMILES: *CN1CC(N2CCC3(CCN(*)CC3)CC2)C1]

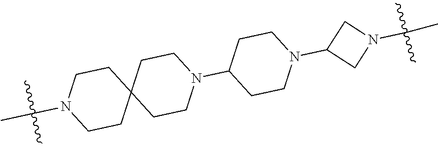 [SMILES: *N1CCC2(CC1)CCN(C1CCN(C3CN(*)C3)CC1)CC2]

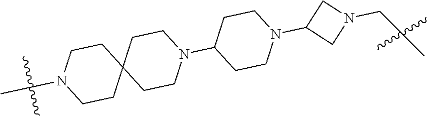 [SMILES: *CN1CC(N2CCC(N3CCC4(CCN(*)CC4)CC3)CC2)C1]

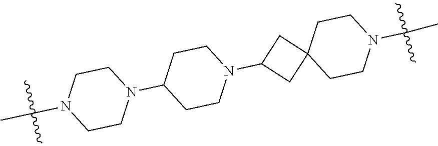 [SMILES: *N1CCN(C2CCN(C3CC4(CCN(*)CC4)C3)CC2)CC1]

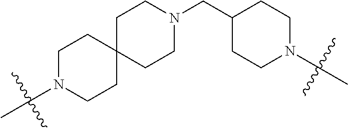 [SMILES: *N1CCC(CN2CCC3(CCN(*)CC3)CC2)CC1]

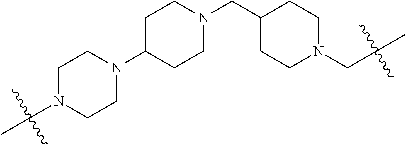 [SMILES: *CN1CCC(CN2CCC(N3CCN(*)CC3)CC2)CC1]

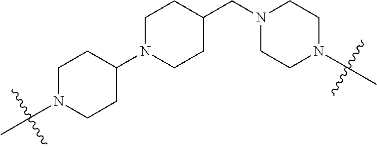 [SMILES: *N1CCC(N2CCC(CN3CCN(*)CC3)CC2)CC1]

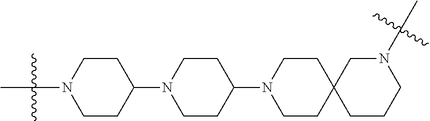 [SMILES: *N1CCC(N2CCC(N3CCC4(CCCN(*)C4)CC3)CC2)CC1]

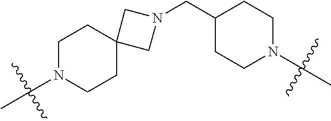 [SMILES: *N1CCC(CN2CC3(CCN(*)CC3)C2)CC1]

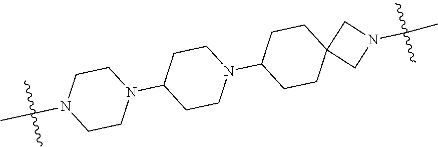 [SMILES: *N1CCN(C2CCN(C3CCC4(CC3)CN(*)C4)CC2)CC1]

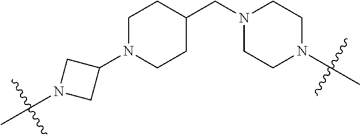 [SMILES: *N1CCN(CC2CCN(C3CN(*)C3)CC2)CC1]

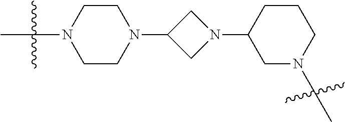 [SMILES: *N1CCN(C2CN(C3CCCN(*)C3)C2)CC1]

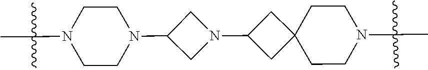 [SMILES: *N1CCN(C2CN(C3CC4(CCN(*)CC4)C3)C2)CC1]

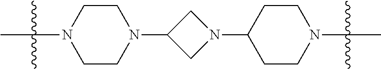 [SMILES: *N1CCC(N2CC(N3CCN(*)CC3)C2)CC1]

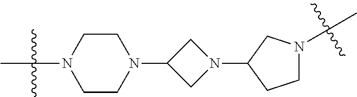 [SMILES: *N1CCN(C2CN(C3CCN(*)C3)C2)CC1]

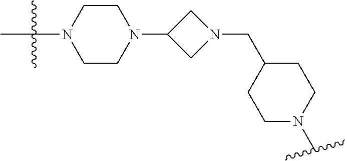 [SMILES: *N1CCC(CN2CC(N3CCN(*)CC3)C2)CC1]

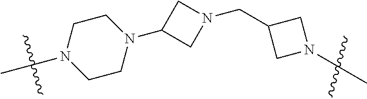 [SMILES: *N1CCN(C2CN(CC3CN(*)C3)C2)CC1]

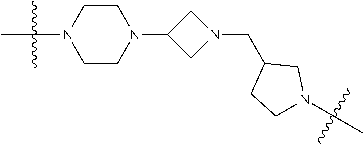 [SMILES: *N1CCN(C2CN(CC3CCN(*)C3)C2)CC1]

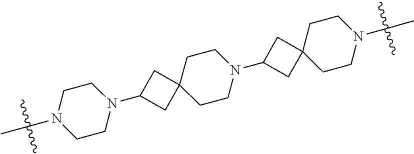 [SMILES: *N1CCN(C2CC3(CCN(C4CC5(CCN(*)CC5)C4)CC3)C2)CC1]

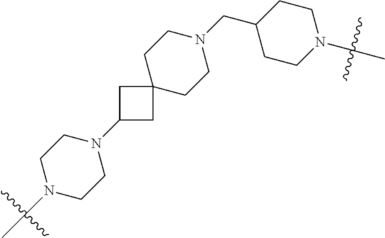 [SMILES: *N1CCC(CN2CCC3(CC2)CC(N2CCN(*)CC2)C3)CC1]

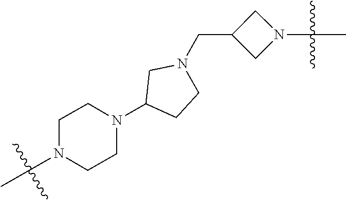 [SMILES: *N1CCN(C2CCN(CC3CN(*)C3)C2)CC1]

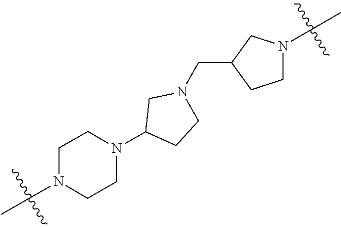 [SMILES: *N1CCN(C2CCN(CC3CCN(*)C3)C2)CC1]

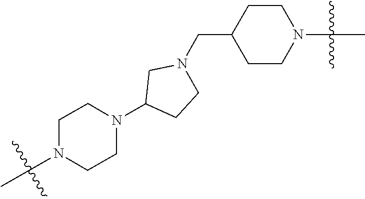 [SMILES: *N1CCC(CN2CCC(N3CCN(*)CC3)C2)CC1]

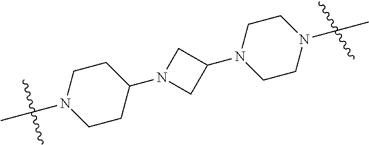 [SMILES: *N1CCC(N2CC(N3CCN(*)CC3)C2)CC1]

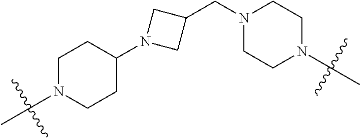 [SMILES: *N1CCC(N2CC(CN3CCN(*)CC3)C2)CC1]

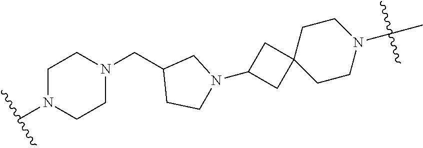 [SMILES: *N1CCN(CC2CCN(C3CC4(CCN(*)CC4)C3)C2)CC1]

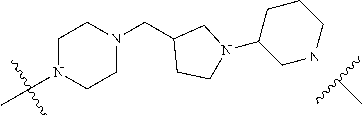

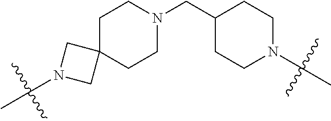 [SMILES: *N1CCC(CN2CCC3(CC2)CN(*)C3)CC1]

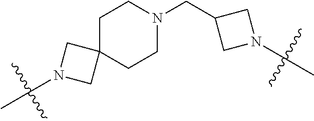 [SMILES: *N1CC(CN2CCC3(CC2)CN(*)C3)C1]

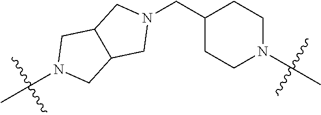 [SMILES: *N1CCC(CN2CC3CN(*)CC3C2)CC1]

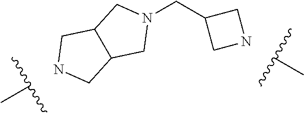

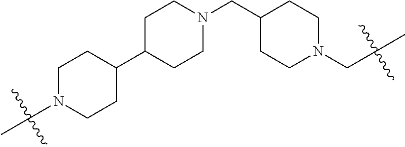 [SMILES: *CN1CCC(CN2CCC(C3CCN(*)CC3)CC2)CC1]

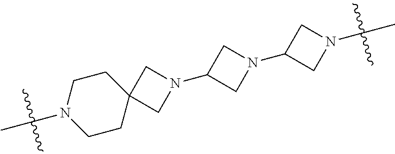 [SMILES: *N1CCC2(CC1)CN(C1CN(C3CN(*)C3)C1)C2]

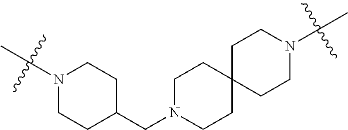 [SMILES: *N1CCC(CN2CCC3(CCN(*)CC3)CC2)CC1]

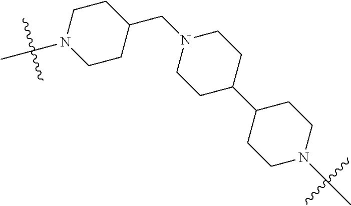 [SMILES: *N1CCC(CN2CCC(C3CCN(*)CC3)CC2)CC1]

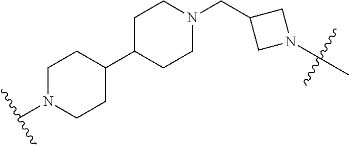 [SMILES: *N1CCC(C2CCN(CC3CN(*)C3)CC2)CC1]

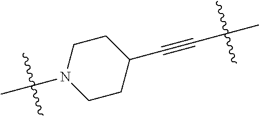 [SMILES: *C#CC1CCN(*)CC1]

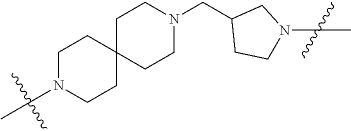 [SMILES: *N1CCC2(CC1)CCN(CC1CCN(*)C1)CC2]

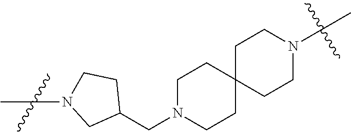 [SMILES: *N1CCC2(CC1)CCN(CC1CCN(*)C1)CC2]

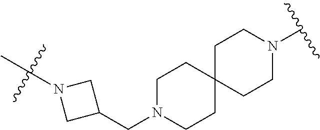 [SMILES: *N1CCC2(CC1)CCN(CC1CN(*)C1)CC2]

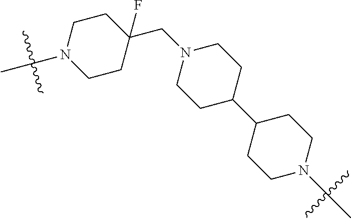 [SMILES: *N1CCC(C2CCN(CC3(F)CCN(*)CC3)CC2)CC1]

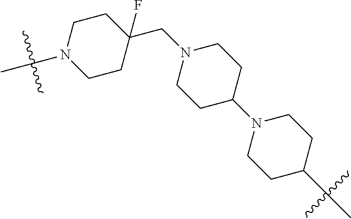 [SMILES: *C1CCN(C2CCN(CC3(F)CCN(*)CC3)CC2)CC1]

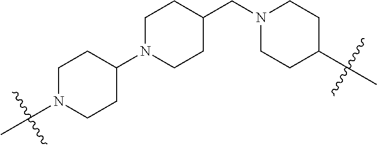 [SMILES: *C1CCN(CC2CCN(C3CCN(*)CC3)CC2)CC1]

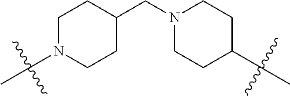 [SMILES: *C1CCN(CC2CCN(*)CC2)CC1]

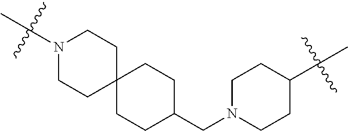 [SMILES: *C1CCN(CC2CCC3(CC2)CCN(*)CC3)CC1]

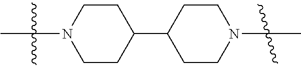 [SMILES: *N1CCC(C2CCN(*)CC2)CC1]

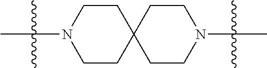 [SMILES: *N1CCC2(CC1)CCN(*)CC2]

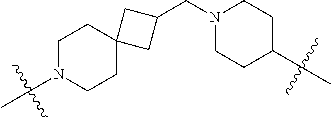 [SMILES: *C1CCN(CC2CC3(CCN(*)CC3)C2)CC1]

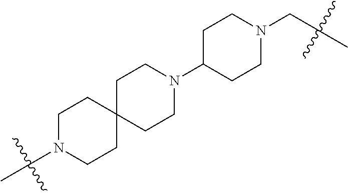 [SMILES: *CN1CCC(N2CCC3(CCN(*)CC3)CC2)CC1]

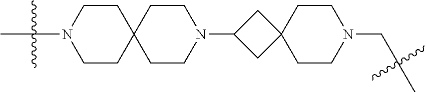 [SMILES: *CN1CCC2(CC1)CC(N1CCC3(CCN(*)CC3)CC1)C2]

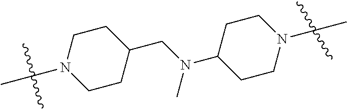 [SMILES: *N1CCC(CN(C)C2CCN(*)CC2)CC1]

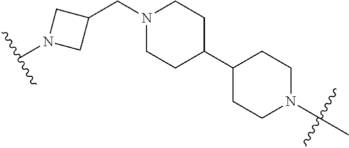 [SMILES: *N1CCC(C2CCN(CC3CN(*)C3)CC2)CC1]

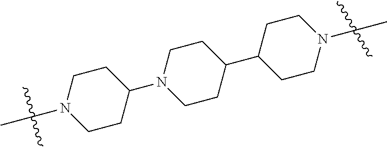 [SMILES: *N1CCC(C2CCN(C3CCN(*)CC3)CC2)CC1]

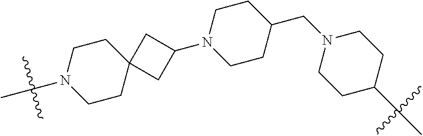 [SMILES: *C1CCN(CC2CCN(C3CC4(CCN(*)CC4)C3)CC2)CC1]

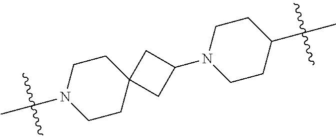 [SMILES: *C1CCN(C2CC3(CCN(*)CC3)C2)CC1]

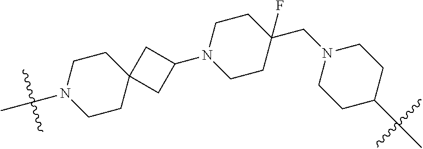 [SMILES: *C1CCN(CC2(F)CCN(C3CC4(CCN(*)CC4)C3)CC2)CC1]

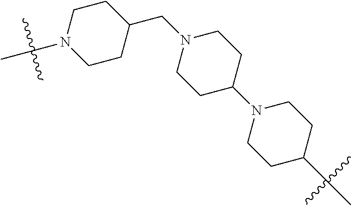 [SMILES: *C1CCN(C2CCN(CC3CCN(*)CC3)CC2)CC1]

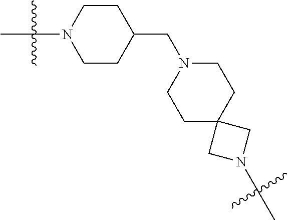 [SMILES: *N1CCC(CN2CCC3(CC2)CN(*)C3)CC1]

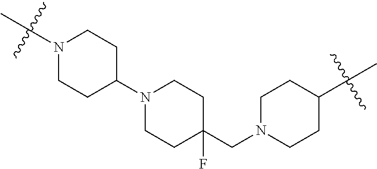 [SMILES: *C1CCN(CC2(F)CCN(C3CCN(*)CC3)CC2)CC1]

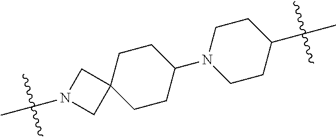 [SMILES: *C1CCN(C2CCC3(CC2)CN(*)C3)CC1]

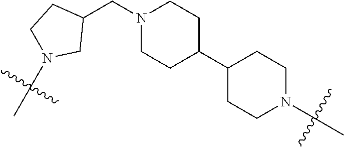 [SMILES: *N1CCC(C2CCN(CC3CCN(*)C3)CC2)CC1]

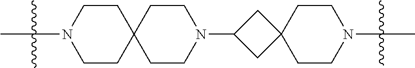 [SMILES: *N1CCC2(CC1)CCN(C1CC3(CCN(*)CC3)C1)CC2]

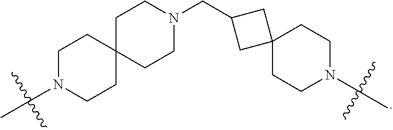 [SMILES: *N1CCC2(CC1)CCN(CC1CC3(CCN(*)CC3)C1)CC2]

TABLE B-1

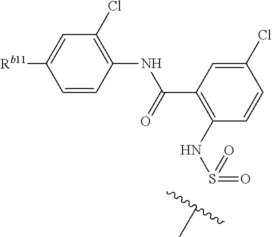

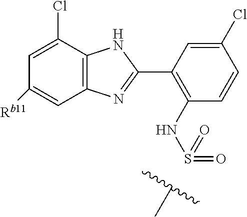

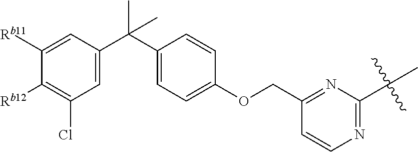

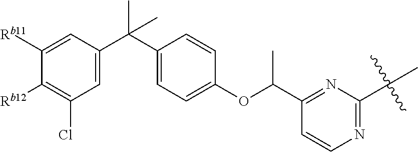

For the compounds in Table B-1, each R^{b11} is independently selected from F, Cl, Br, CF₃, CN, or NO₂, preferably CF₃ or CN; each R^{b12} is independently selected from H, OCH₃, OCD₃, OCH₂CH₃, or OCH₂CH₂Cl.

TABLE B-2

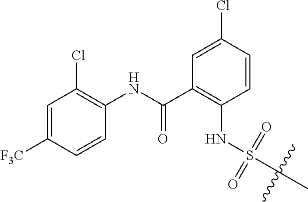 [SMILES: *S(=O)(=O)Nc1ccc(Cl)cc1C(=O)Nc1ccc(C(F)(F)F)cc1Cl]

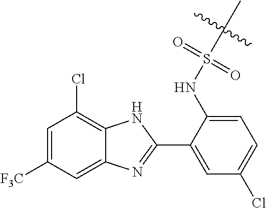 [SMILES: *S(=O)(=O)Nc1ccc(Cl)cc1-c1nc2cc(C(F)(F)F)cc(Cl)c2[nH]1]

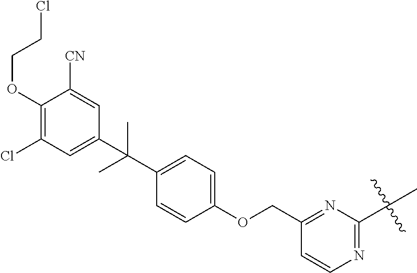 [SMILES: *c1nccc(COc2ccc(C(C)(C)c3cc(Cl)c(OCCCl)c(C#N)c3)cc2)n1]

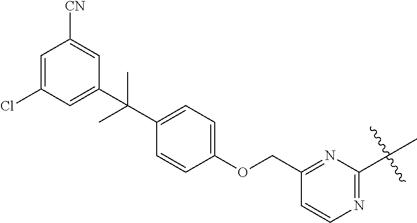 [SMILES: *c1nccc(COc2ccc(C(C)(C)c3cc(Cl)cc(C#N)c3)cc2)n1]

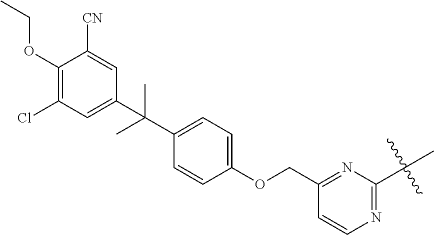 [SMILES: *c1nccc(COc2ccc(C(C)(C)c3cc(Cl)c(OCC)c(C#N)c3)cc2)n1]

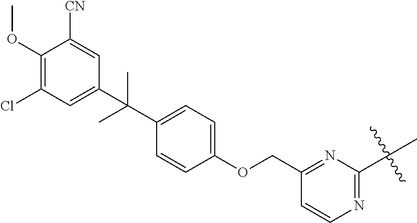 [SMILES: *c1nccc(COc2ccc(C(C)(C)c3cc(Cl)c(OC)c(C#N)c3)cc2)n1]

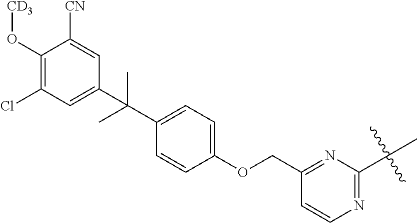 [SMILES: *c1nccc(COc2ccc(C(C)(C)c3cc(Cl)c(OC([2H])([2H])[2H])c(C#N)c3)cc2)n1]

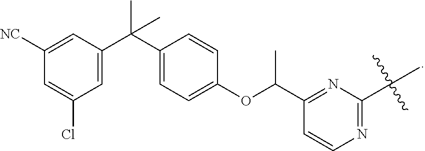 [SMILES: *c1nccc(C(C)Oc2ccc(C(C)(C)c3cc(Cl)cc(C#N)c3)cc2)n1]

TABLE K-1

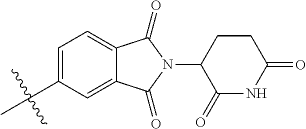 [SMILES: *c1ccc2c(c1)C(=O)N(C1CCC(=O)NC1=O)C2=O]

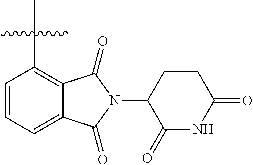 [SMILES: *c1cccc2c1C(=O)N(C1CCC(=O)NC1=O)C2=O]

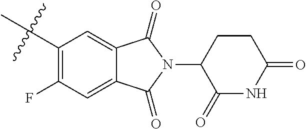 [SMILES: *c1cc2c(cc1F)C(=O)N(C1CCC(=O)NC1=O)C2=O]

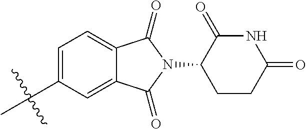 [SMILES: *c1ccc2c(c1)C(=O)N([C@H]1CCC(=O)NC1=O)C2=O]

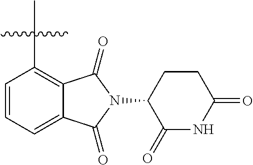 [SMILES: *c1cccc2c1C(=O)N([C@@H]1CCC(=O)NC1=O)C2=O]

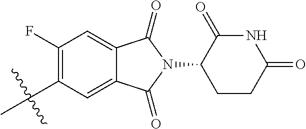 [SMILES: *c1cc2c(cc1F)C(=O)N([C@H]1CCC(=O)NC1=O)C2=O]

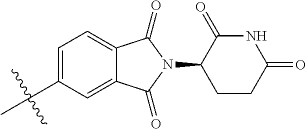 [SMILES: *c1ccc2c(c1)C(=O)N([C@@H]1CCC(=O)NC1=O)C2=O]

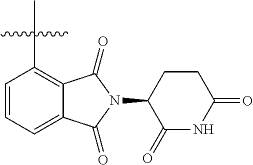 [SMILES: *c1cccc2c1C(=O)N([C@H]1CCC(=O)NC1=O)C2=O]

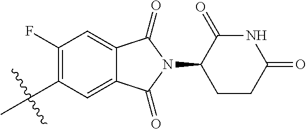 [SMILES: *c1cc2c(cc1F)C(=O)N([C@@H]1CCC(=O)NC1=O)C2=O]

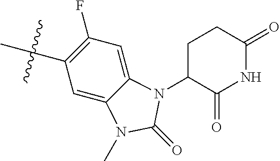 [SMILES: *c1cc2c(cc1F)n(C1CCC(=O)NC1=O)c(=O)n2C]

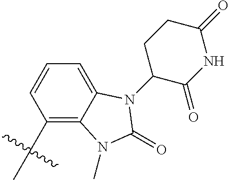 [SMILES: *c1cccc2c1n(C)c(=O)n2C1CCC(=O)NC1=O]

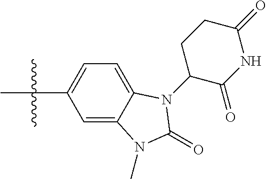 [SMILES: *c1ccc2c(c1)n(C)c(=O)n2C1CCC(=O)NC1=O]

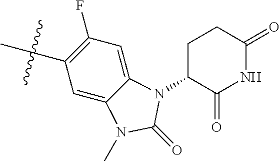 [SMILES: *c1cc2c(cc1F)n([C@@H]1CCC(=O)NC1=O)c(=O)n2C]

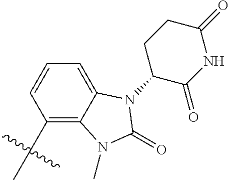 [SMILES: *c1cccc2c1n(C)c(=O)n2[C@@H]1CCC(=O)NC1=O]

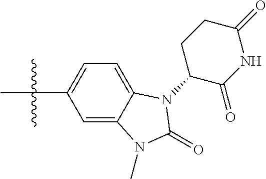 [SMILES: *c1ccc2c(c1)n(C)c(=O)n2[C@@H]1CCC(=O)NC1=O]

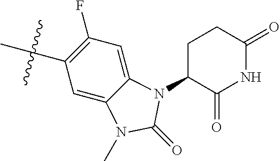 [SMILES: *c1cc2c(cc1F)n([C@H]1CCC(=O)NC1=O)c(=O)n2C]

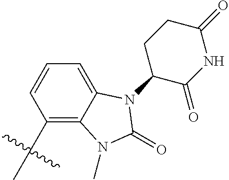 [SMILES: *c1cccc2c1n(C)c(=O)n2[C@H]1CCC(=O)NC1=O]

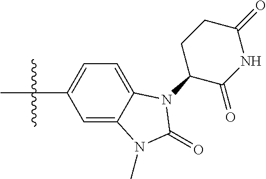 [SMILES: *c1ccc2c(c1)n(C)c(=O)n2[C@H]1CCC(=O)NC1=O]

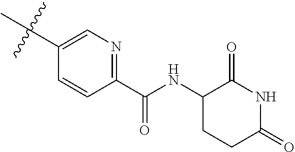 [SMILES: *c1ccc(C(=O)NC2CCC(=O)NC2=O)nc1]

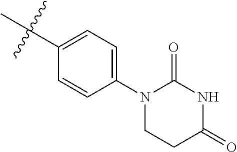 [SMILES: *c1ccc(N2CCC(=O)NC2=O)cc1]

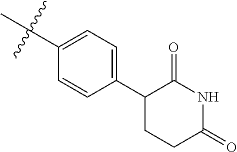 [SMILES: *c1ccc(C2CCC(=O)NC2=O)cc1]

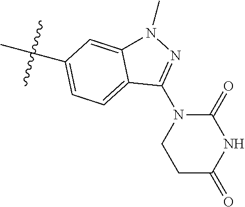 [SMILES: *c1ccc2c(N3CCC(=O)NC3=O)nn(C)c2c1]

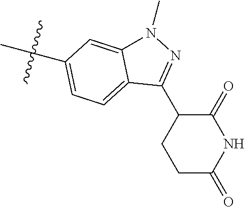 [SMILES: *c1ccc2c(C3CCC(=O)NC3=O)nn(C)c2c1]

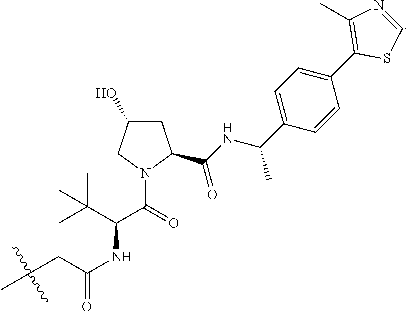 [SMILES: *CC(=O)N[C@H](C(=O)N1C[C@H](O)C[C@H]1C(=O)N[C@@H](C)c1ccc(-c2scnc2C)cc1)C(C)(C)C]

In some embodiments, the pharmaceutically acceptable salt of the compound is trifluoroacetate thereof.

The present invention relates to a pharmaceutical composition, comprising the above-mentioned compound in the present invention or the stereoisomer, deuterated compound, solvate, prodrug, metabolite, pharmaceutically acceptable salt or co-crystal thereof, and a pharmaceutically acceptable carrier.

The present invention relates to the use of the above-mentioned compound in the present invention or the stereoisomer, deuterated compound, solvate, prodrug, metabolite, pharmaceutically acceptable salt or co-crystal thereof in the preparation of a medicament for treating a disease related to the activity or expression level of an AR or AR splice variant.

The present invention relates to the use of the above-mentioned compound in the present invention or the stereoisomer, deuterated compound, solvate, prodrug, metabolite, pharmaceutically acceptable salt or co-crystal thereof in the preparation of a medicament for treating a disease related to the inhibition or degradation of an AR or AR splice variant.

The present invention relates to the use of the above-mentioned compound in the present invention or the stereoisomer, deuterated compound, solvate, prodrug, metabolite, pharmaceutically acceptable salt or co-crystal thereof, characterized in that the disease is selected from an autoimmune disease, an inflammatory disease or cancer (preferably, prostate cancer).

The present invention relates to a method for the inhibition or degradation of an AR or AR splice variant, comprising a step of contacting the above-mentioned compound or the stereoisomer, deuterated compound, solvate, prodrug, metabolite, pharmaceutically acceptable salt or co-crystal thereof with a cell.

In some embodiments, the cell is from a subject.

The present invention relates to a method for treating a disease related to abnormal activity or expression level of an AR or AR splice variant, comprising a step of administering to a subject in need thereof an effective amount of the above-mentioned compound or the stereoisomer, deuterated compound, solvate, prodrug, metabolite, pharmaceutically acceptable salt or co-crystal thereof.

In some embodiments, the disease is selected from an autoimmune disease, an inflammatory disease or cancer. In some embodiments, the cancer is prostate cancer, such as enzalutamide-resistant prostate cancer.

The present invention relates to the above-mentioned compound or the stereoisomer, deuterated compound, solvate, prodrug, metabolite, pharmaceutically acceptable salt or co-crystal thereof, for use in the inhibition or degradation of an AR or AR splice variant.

The present invention relates to the above-mentioned compound or the stereoisomer, deuterated compound, solvate, prodrug, metabolite, pharmaceutically acceptable salt or co-crystal thereof, for use in the treatment of a disease related to abnormal activity or expression level of an AR or AR splice variant.

In some embodiments, the disease is selected from an autoimmune disease, an inflammatory disease or cancer. In some embodiments, the cancer is prostate cancer, such as enzalutamide-resistant prostate cancer.

Unless stated to the contrary, the terms used in the description and claims have the following meanings.

The carbon, hydrogen, oxygen, sulfur, nitrogen or F, Cl, Br, I involved in the groups and compounds of the present invention all comprise their isotopes, and the carbon, hydrogen, oxygen, sulfur or nitrogen involved in the groups and compounds of the present invention is optionally further substituted with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

“Halogen” refers to F, Cl, Br or I.

“Halogen-substituted” refers to F, Cl, Br or I substitution, including but not limited to a substitution with 1 to 10 substituents selected from F, Cl, Br or I, a substitution with 1 to 6 substituents selected from F, Cl, Br or I, or a substitution with 1 to 4 substituents selected from F, Cl, Br or I. “Halogen-substituted” is referred to simply as “halo”.

“Alkyl” refers to a substituted or unsubstituted linear or branched saturated aliphatic hydrocarbyl group, including but not limited to an alkyl group of 1 to 20 carbon atoms, an alkyl group of 1 to 8 carbon atoms, an alkyl group of 1 to 6 carbon atoms, or an alkyl group of 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof. The definition of the “alkyl” herein is consistent with this definition. Alkyl group can be monovalent, divalent, trivalent or tetravalent.

“Hydrocarbyl” refers to a substituted or unsubstituted linear or branched saturated or unsaturated group consisting of carbon and hydrogen atoms. Hydrocarbyl group can be monovalent, divalent, trivalent or tetravalent.

“Heteroalkyl” refers to a substituted or unsubstituted alkyl group in which one or more (including but not limited to 2, 3, 4, 5 or 6) carbon atoms are replaced by heteroatoms (including but not limited to N, O or S). Non-limiting examples include —X(CH₂)v-X(CH₂)v-X(CH₂)v-H (v is an integer from 1 to 5; X is independently selected from bonds or heteroatoms; heteroatoms include but are not limited to N, O or S; at least one X is selected from heteroatoms; and N or S in heteroatoms can be oxidized to various oxidation states). Heteroalkyl group can be monovalent, divalent, trivalent or tetravalent.

“Alkylene” refers to a substituted or unsubstituted linear or branched divalent saturated hydrocarbyl group, including —(CH₂)ᵥ— (v is an integer from 1 to 10), and examples of alkylene include, but are not limited to, methylene, ethylene, propylene, butylene, etc.

“Heteroalkylene” refers to a substituted or unsubstituted alkylene group in which one or more (including but not limited to 2, 3, 4, 5 or 6) carbon atoms are replaced by heteroatoms (including but not limited to N, O or S). Non-limiting examples include —X(CH₂)v-X(CH₂)v-X(CH₂)v-, wherein v is an integer from 1 to 5, each X is independently selected from a bond, N, O or S, and at least one X is selected from N, O or S.

“Cycloalkyl” refers to a substituted or unsubstituted saturated carbocyclic hydrocarbyl group, usually having from 3 to 10 carbon atoms, and non-limiting examples include cyclopropyl, cyclobutyl, cyclopentyl, cyclohexyl, cycloheptyl, etc. The “cycloalkyl” herein is as defined above. Cycloalkyl group can be monovalent, divalent, trivalent or tetravalent.

“Heterocycloalkyl” refers to a substituted or unsubstituted saturated heteroatom-containing cyclic hydrocarbyl group, including but not limited to 3 to 10 atoms, 3 to 8 atoms, or 1 to 3 heteroatoms selected from N, O or S. N and S selectively substituted in the heterocycloalkyl ring can be oxidized to various oxidation states. Heterocycloalkyl can be connected to a heteroatom or a carbon atom; heterocycloalkyl can be connected to an aromatic ring or a non-aromatic ring; and heterocycloalkyl can be connected to a bridged ring or a spiro ring. Non-limiting examples include oxiranyl, azacyclopropyl, oxetanyl, azetidinyl, tetrahydrofuranyl, tetrahydro-2H-pyranyl, dioxolanyl, dioxanyl, pyrrolidinyl, piperidinyl, imidazolidinyl, oxazolidinyl, oxazinanyl, morpholinyl, hexahydropyrimidinyl or piperazinyl. Heterocycloalkyl group can be monovalent, divalent, trivalent or tetravalent.

“Alkenyl” refers to a substituted or unsubstituted linear or branched unsaturated hydrocarbyl group, having at least 1, usually 1, 2 or 3 carbon-carbon double bonds, with a main chain including but not limited to 2 to 10, 2 to 6, or 2 to 4 carbon atoms. Examples of alkenyl include, but are not limited to, ethenyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, etc. The definition of the “alkenyl” herein is consistent with this definition. Alkenyl group can be monovalent, divalent, trivalent or tetravalent.

“Alkynyl” refers to a substituted or unsubstituted linear or branched monovalent unsaturated hydrocarbyl group, having at least 1, usually 1, 2 or 3 carbon-carbon triple bonds, with a main chain including but not limited to 2 to 10 carbon atoms, 2 to 6 carbon atoms, or 2 to 4 carbon atoms. Examples of alkynyl include, but are not limited to, ethynyl, propargyl, 1-propynyl, 2-propynyl, 1-butynyl, 2-butynyl, 3-butynyl, 1-pentynyl, 2-pentynyl, 3-pentynyl, 4-pentynyl, 1-methyl-1-butynyl, 2-methyl-1-butynyl, 2-methyl-3-butynyl, 1-hexynyl, 2-hexynyl, 3-hexynyl, 4-hexynyl, 5-hexynyl, 1-methyl-1-pentynyl, 2-methyl-1-pentynyl, 1-heptynyl, 2-heptynyl, 3-heptynyl, 4-heptynyl, 1-octynyl, 3-octynyl, 1-nonynyl, 3-nonynyl, 1-decynyl, 4-decynyl, etc. Alkynyl group can be monovalent, divalent, trivalent or tetravalent.

“Alkoxy” refers to a substituted or unsubstituted —O-alkyl group. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy.

“Carbocyclyl” or “carbocycle” refers to a substituted or unsubstituted saturated or unsaturated aromatic ring or non-aromatic ring, wherein the aromatic ring or non-aromatic ring can be 3- to 8-membered monocyclic ring, 4- to 12-membered bicyclic ring or 10- to 15-membered tricyclic ring system. Carbocyclyl can be connected to an aromatic ring or a non-aromatic ring, wherein the aromatic ring or non-aromatic ring is optionally a monocyclic ring, a bridged ring or a spiro ring. Non-limiting examples include cyclopropane, cyclobutane, cyclopentane, cyclohexane, cycloheptane, 1-cyclopentyl-1-enyl, 1-cyclopentyl-2-enyl, 1-cyclopentyl-3-enyl, cyclohexyl, 1-cyclohexyl-2-enyl, 1-cyclohexyl-3-enyl, cyclohexenyl, a benzene ring, a naphthalene ring,

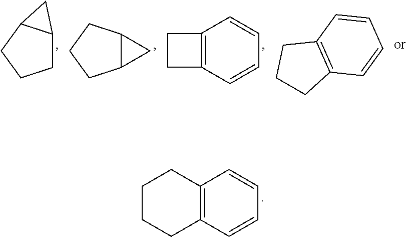

“Carbocyclyl” or “carbocycle” can be monovalent, divalent, trivalent or tetravalent.

“Heterocyclyl” or “heterocycle” refers to a substituted or unsubstituted saturated or unsaturated aromatic ring or non-aromatic ring, wherein the aromatic ring or non-aromatic ring can be 3- to 8-membered monocyclic ring, 4- to 12-membered bicyclic ring or 10- to 15-membered tricyclic ring system, and contains one or more (including but not limited to 2, 3, 4 or 5) heteroatoms selected from N, O or S, and the selectively substituted N and S in the heterocyclyl ring can be oxidized to various oxidation states. Heterocyclyl can be connected to a heteroatom or a carbon atom; heterocyclyl can be connected to an aromatic ring or a non-aromatic ring; and heterocyclyl can be connected to a bridged ring or a spiro ring. Non-limiting examples include oxiranyl, azacyclopropyl, oxetanyl, azetidinyl, 1,3-dioxolanyl, 1,4-dioxolanyl, 1,3-dioxanyl, azacycloheptyl, pyridyl, furyl, thienyl, pyranyl, N-alkylpyrrolyl, pyrimidinyl, pyrazinyl, pyridazinyl, imidazolyl, piperidyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, tetrahydrothiazolyl, tetrahydropyranyl, benzoimidazolyl, benzopyridyl, pyrrolopyridinyl, benzodihydrofuryl, pyrrolyl, pyrazolyl, thiazolyl, oxazolyl, pyrazinyl, indazolyl, benzothienyl, benzofuryl, benzopyrrolyl, benzoimidazolyl, benzothiazolyl, benzoxazolyl, benzopyridyl, benzopyrimidinyl, benzopyrazinyl, piperazinyl, azabicyclo[3.2.1]octanyl, azabicyclo[5.2.0]nonanyl, oxatricyclo[5.3.1.1]dodecyl, azaadamantyl, oxaspiro[3.3]heptanyl,

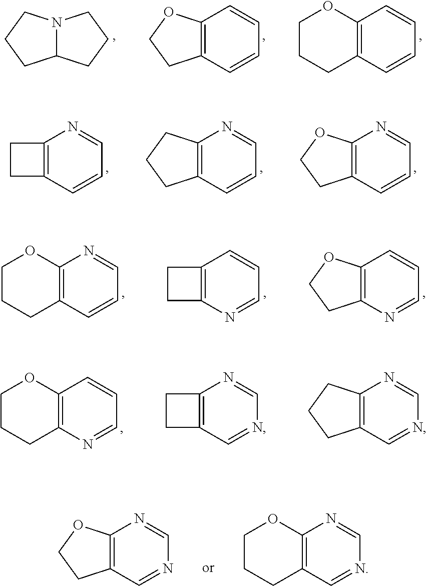

“Heterocyclyl” or “heterocycle” can be monovalent, divalent, trivalent or tetravalent.

“Spiro ring” or “spiro ring group” refers to a polycyclic group that shares one atom (called a spiro atom) between substituted or unsubstituted monocyclic rings. The number of ring atoms in the spiro ring system includes but is not limited to 5 to 20, 6 to 14, 6 to 12, or 6 to 10, wherein one or more rings may contain 0 or more (including but not limited to 1, 2, 3 or 4) double bonds, and can optionally contain 0 to 5 heteroatoms selected from N, O or S(═O)ₙ.

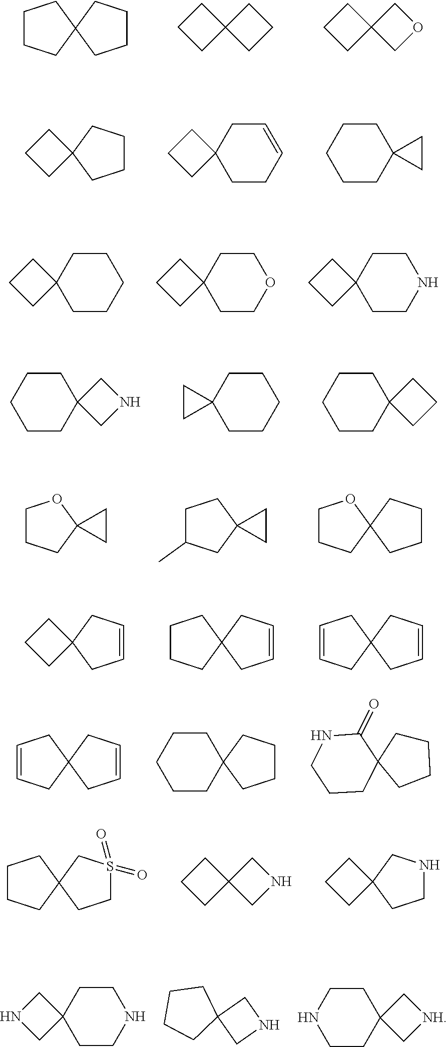

“Spiro ring” or “spiro ring group” can be monovalent, divalent, trivalent or tetravalent.

“Fused ring” or “fused ring group” refers to a polycyclic group in which each ring in the system shares an adjacent pair of atoms with other rings in the system, wherein one or more rings may contain 0 or more (including but not limited to 1, 2, 3 or 4) double bonds, and may be substituted or unsubstituted, and each ring in the fused ring system may contain 0 to 5 heteroatoms or groups containing heteroatoms (including but not limited to N, S(═O)ₙ or O, wherein n is 0, 1 or 2). The number of ring atoms in the fused ring system includes but is not limited to 5 to 20, 5 to 14, 5 to 12, or 5 to 10. Non-limiting examples include:

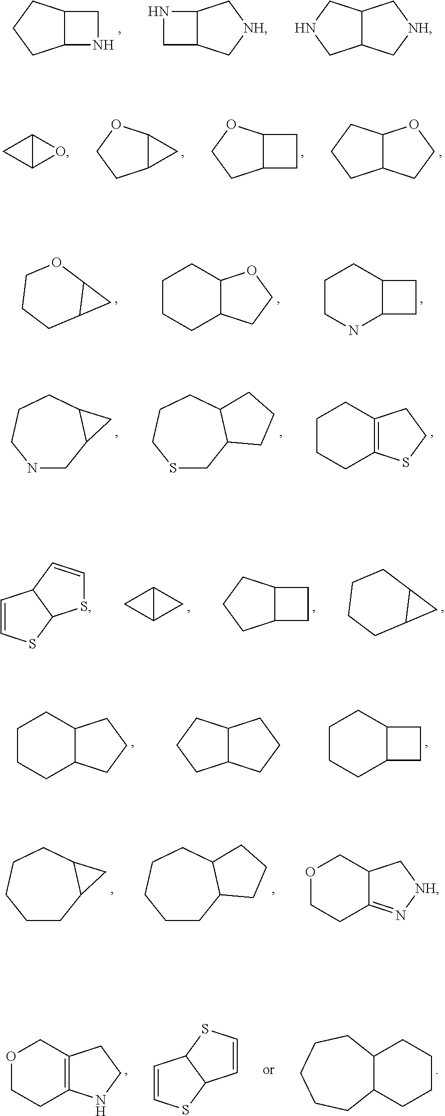

“Fused ring” or “fused ring group” can be monovalent, divalent, trivalent or tetravalent.

“Bridged ring” or “bridged ring group” refers to a substituted or unsubstituted polycyclic group containing any two atoms that are not directly connected, and may contain 0 or more double bonds. Any ring in the fused ring system may contain 0 to 5 groups selected from heteroatoms or groups containing heteroatoms (including but not limited to N, S(═O)n or O, wherein n is 0, 1 or 2). The number of ring atoms includes but is not limited to 5 to 20, 5 to 14, 5 to 12 or 5 to 10. Non-limiting examples include

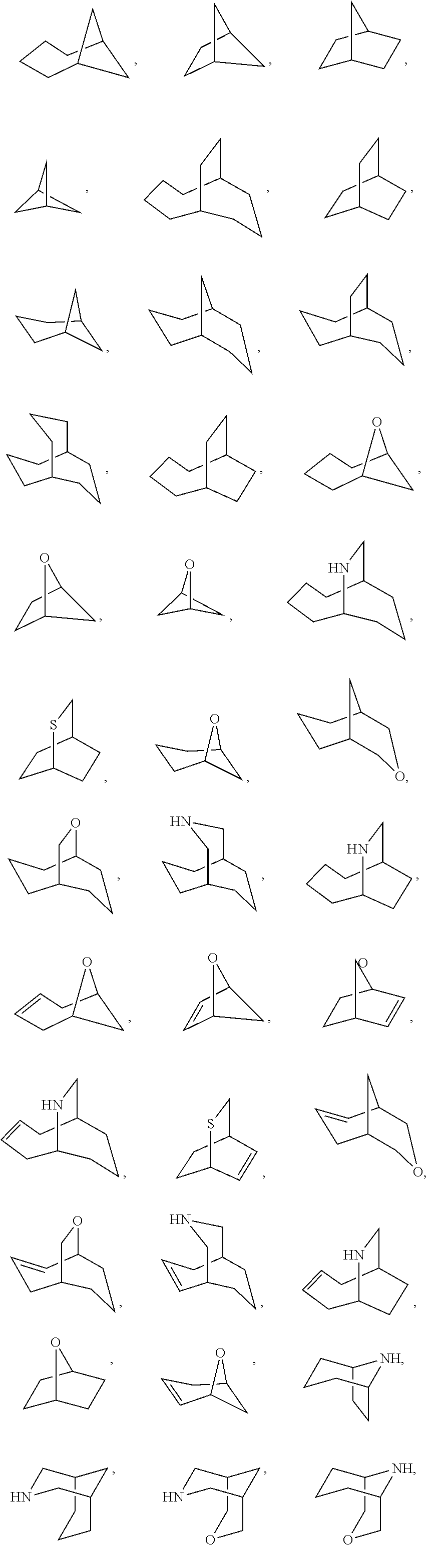

cubane or adamantane. “Bridged ring” or “bridged ring group” can be monovalent, divalent, trivalent or tetravalent.

“Carbospiro ring”, “spiro ring carbocyclyl”, “spirocarbocyclyl” or “carbospiro ring group” refers to a “spiro ring” with a ring system consisting only of carbon atoms. The definition of the “carbospiro ring”, “spiro ring carbocyclyl”, “spirocarbocyclyl” or “carbospiro ring group” herein is consistent with that of a spiro ring.

“Carbo-fused ring”, “fused ring carbocyclyl”, “fused carbocyclyl” or “carbo-fused ring group” refers to a “fused ring” with a ring system consisting only of carbon atoms. The definition of the “carbo-fused ring”, “fused ring carbocyclyl”, “fused carbocyclyl” or “carbo-fused ring group” herein is consistent with that of a fused ring.

“Carbo-bridged ring”, “bridged ring carbocyclyl”, “bridged carbocyclyl” or “carbo-bridged ring group” refers to a “bridged ring” with a ring system consisting only of carbon atoms. The definition of the “carbo-bridged ring”, “bridged ring carbocyclyl”, “bridged carbocyclyl” or “carbo-bridged ring group” herein is consistent with that of a bridged ring.

“Mono-heterocyclic ring”, “monocyclic heterocyclyl” or “mono-heterocyclic ring group” refers to “heterocyclyl” or “heterocycle” with a monocyclic system. The definition of the “heterocyclyl”, “monocyclic heterocyclyl” or “mono-heterocyclic ring group” herein is consistent with that of heterocycle.

“Fused heterocyclic ring”, “fused heterocyclic ring group”, “fused ring heterocyclyl” or “fused heterocyclic ring group” refers to a “fused ring” containing a heteroatom. The definition of the “fused heterocyclic ring”, “fused heterocyclic ring group”, “fused ring heterocyclyl” or “fused heterocyclic ring group” herein is consistent with that of a fused ring.

“Spiro-heterocyclic ring”, “spiro-heterocyclic ring group”, “spiro ring heterocyclyl” or “spiro-heterocyclic ring group” refers to a “spiro ring” containing a heteroatom. The definition of the “spiro-heterocyclic ring”, “spiro-heterocyclic ring group”, “spiro ring heterocyclyl” or “spiro-heterocyclic ring group” herein is consistent with that of a spiro ring.

“Bridged-heterocyclic ring”, “bridged-heterocyclic ring group”, “bridged ring heterocyclyl” or “bridged-heterocyclic ring group” refers to a “bridged ring” containing a heteroatom. The definition of the “bridged-heterocyclic ring”, “bridged-heterocyclic ring group”, “bridged ring heterocyclyl” or “bridged-heterocyclic ring group” herein is consistent with that of a bridged ring.

“Aryl” or “aromatic ring” refers to a substituted or unsubstituted aromatic hydrocarbyl group with a monocyclic ring or a fused ring, wherein the number of ring atoms in the aromatic ring includes but is not limited to 6 to 18, 6 to 12 or 6 to 10 carbon atoms. The aryl ring may be fused to a saturated or unsaturated carbocycle or heterocycle, wherein the ring connected to the parent structure is an aryl ring. Non-limiting examples include a benzene ring, a naphthalene ring, or

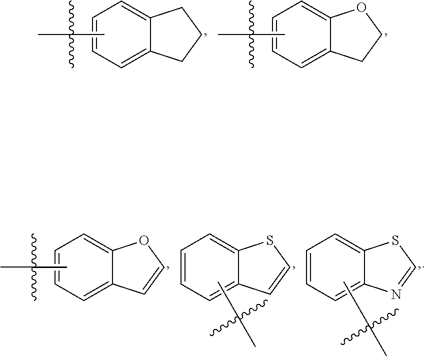

“Aryl” or “aromatic ring” can be monovalent, divalent, trivalent or tetravalent. When divalent, trivalent or tetravalent, the point of connection is on the aryl ring.

“Heteroaryl” or “heteroaromatic ring” refers to a substituted or unsubstituted aromatic hydrocarbyl group containing 1 to 5 heteroatoms or groups containing heteroatoms (including but not limited to N, O or S(═O)ₙ, wherein n is 0, 1 or 2), wherein the number of ring atoms in the heteroaromatic ring includes but is not limited to 5-15, 5-10 or 5-6. Non-limiting examples of heteroaryl include, but are not limited to pyridyl, furyl, thienyl, pyridyl, pyranyl, N-alkylpyrrolyl, pyrimidinyl, pyrazinyl, pyridazinyl, imidazolyl, benzopyrazole, benzimidazole, benzopyridine, pyrrolopyridine, etc. The heteroaryl ring may be fused to a saturated or unsaturated carbocycle or heterocycle, wherein the ring connected to the parent structure is an heteroaryl ring. Non-limiting examples include

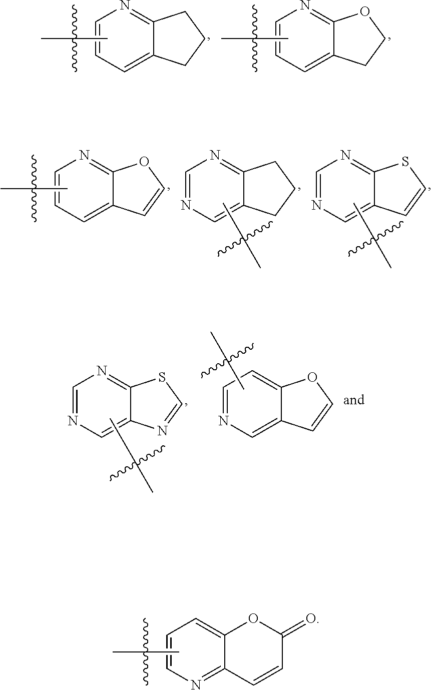

The definition of the “heteroaryl” herein is consistent with this definition. Heteroaryl group can be monovalent, divalent, trivalent or tetravalent. When divalent, trivalent or tetravalent, the point of connection is on the heteroaryl ring.

“5-membered ring fused 5-membered heteroaromatic ring” refers to a 5 fused 5-membered fused heteroaromatic ring, wherein at least one of the two fused rings contains at least one heteroatom (including but not limited to O, S or N), and the entire group is aromatic. Non-limiting examples include a pyrrolopyrrole ring, a pyrazolopyrrole ring, a pyrazolopyrazole ring, a pyrrolofuran ring, a pyrazolofuran ring, a pyrrolothiophene ring and a pyrazolothiophene ring.

“5 fused 6-membered heteroaromatic ring” refers to a 5 fused 6-membered fused heteroaromatic ring, wherein at least one of the two fused rings contains at least one heteroatom (including but not limited to O, S or N), and the entire group is aromatic. Non-limiting examples include a benzo 5-membered heteroaryl and 6-membered heteroaromatic ring fused 5-membered heteroaromatic ring.

“Substitution” or “substituted” refers to a substitution with 1 or more (including but not limited to 2, 3, 4 or 5) substituents including but not limited to H, F, Cl, Br, I, alkyl, cycloalkyl, alkoxy, haloalkyl, mercaptan, hydroxyl, nitro, mercapto, amino, cyano, isocyano, aryl, heteroaryl, heterocyclyl, bridged ring group, spiro ring group, fused ring group, hydroxyalkyl, ═O, carbonyl, aldehyde, carboxylic acid, carboxylate, —(CH₂)ₘ—C(═O)—R^{a}, —O—(CH₂)ₘ—C(═O)—R^{a}, —(CH₂)ₘ—C(═O)—NR^{b}R^{c}, —(CH₂)ₘS(═O)ₙR^{a}, —(CH₂)ₘ-alkenyl-R^{a}, OR^{d} or —(CH₂)ₘ-alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, R^{b} and R^{c} may form a five- or six-membered cycloalkyl or heterocyclyl; each R^{a} or R^{d} is independently selected from aryl, heteroaryl, alkyl, alkoxy, cycloalkyl, heterocyclyl, carbonyl, ester group, bridged ring group, spiro ring group or fused ring group.

“Containing 1 to 5 heteroatoms selected from O, S or N” means containing 1, 2, 3, 4 or 5 heteroatoms selected from O, S or N.

“Substituted with 0 to X substituents selected from . . . ” means substituted with 0, 1, 2, 3 . . . X substituents selected from . . . , wherein X is selected from any integer between 1 and 10. For example, “substituted with 0 to 4 substituents selected from . . . ” means substituted with 0, 1, 2, 3 or 4 substituents selected from . . . For example, “substituted with 0 to 5 substituents selected from . . . ” means substituted with 0, 1, 2, 3, 4 or 5 substituents selected from. . . . For example, “bridged-heterocyclic ring is optionally further substituted with 0 to 4 substituents selected from H or F” means that the bridged-heterocyclic ring is optionally further substituted with 0, 1, 2, 3 or 4 substituents selected from H or F.

An X- to Y-membered ring (X is selected from an integer less than Y and greater than or equal to 3, and Y is selected from any integer between 4 and 12) includes X+1-, X+2-, X+3-, X+4-, . . . , Y-membered rings. Rings include a heterocycle, a carbocycle, an aromatic ring, aryl, heteroaryl, cycloalkyl, a mono-heterocyclic ring, a fused heterocyclic ring, a spiro-heterocyclic ring or a bridged-heterocyclic ring. For example, a “4- to 7-membered mono-heterocyclic ring” refers to a 4-, 5-, 6- or 7-membered mono-heterocyclic ring, and a “5- to 10-membered fused heterocyclic ring” refers to a 5-, 6-, 7-, 8-, 9- or 10-membered fused heterocyclic ring.

“Pharmaceutically acceptable salt” or “pharmaceutically acceptable salt thereof” refers to a salt of the compound of the present invention, which salt maintains the biological effectiveness and characteristics of a free acid or a free base, and is obtained by reacting the free acid with a non-toxic inorganic base or organic base, or reacting the free base with a non-toxic inorganic acid or organic acid.

“Pharmaceutical composition” refers to a mixture of one or more compounds of the present invention, or stereoisomers, tautomers, deuterated compounds, solvates, prodrugs, metabolites, pharmaceutically acceptable salts or co-crystals thereof and other chemical components, wherein “other chemical components” refer to pharmaceutically acceptable carriers, excipients and/or one or more other therapeutic agents.

“Carrier” refers to a material that does not cause significant irritation to an organism and does not eliminate the biological activity and characteristics of a compound administered.

“Excipient” refers to an inert substance added to a pharmaceutical composition to facilitate the administration of a compound. Non-limiting examples include calcium carbonate, calcium phosphate, sugar, starch, cellulose derivatives (including microcrystalline cellulose), gelatin, vegetable oils, polyethylene glycols, diluents, granulating agents, lubricants, adhesives and disintegrants.

“Prodrug” refers to a compound that can be converted into the compound of the present invention with the biological activity by metabolism in vivo. The prodrug of the present invention is prepared by modifying an amino or carboxyl group in the compound of the present invention, and the modification can be removed by conventional operations or in vivo to obtain a parent compound. When the prodrug of the present invention is administered to a mammalian individual, the prodrug is split to form a free amino or carboxyl group.

“Co-crystal” refers to a crystal formed by the combination of active pharmaceutical ingredient (API) and co-crystal former (CCF) under the action of hydrogen bonds or other non-covalent bonds. The pure states of API and CCF are both solid at room temperature, and there is a fixed stoichiometric ratio among various components. The co-crystal is a multi-component crystal, which includes both a binary co-crystal formed between two neutral solids and a multi-element co-crystal formed between a neutral solid and a salt or solvate.

“Animal” is meant to include mammals, such as humans, companion animals, zoo animals, and domestic animals, preferably humans, horses, or dogs.

“Stereoisomer” refers to an isomer produced as a result of different spatial arrangement of atoms in molecules, including cis-trans isomers, enantiomers and conformational isomers.

“Tautomer” refers to a functional group isomer produced by the rapid movement of an atom in two positions in a molecule, such as keto-enol isomerization and amide-imino alcohol isomerization.

“Optional” or “optionally” or “selective” or “selectively” means that the events or conditions subsequently described may but not necessarily occur, and the description includes the case where the events or conditions occur and do not occur. For example, “heterocyclyl selectively substituted with alkyl” means that the alkyl may but not necessarily exist, and the description includes the case where the heterocyclyl is substituted with alkyl and the case where the heterocyclyl is not substituted with alkyl.

“IC₅₀” refers to the concentration of a medicament or inhibitor required to inhibit half of a given biological process (or a component of the process such as an enzyme, a receptor and a cell).

### DETAILED DESCRIPTION OF EMBODIMENTS

To achieve the objectives of the present invention, according to organic synthesis techniques known to those skilled in the art, and starting from commercially available chemicals and/or compounds described in chemical documents, the prepared compounds, “commercially available chemicals”, for use in the reactions described herein are obtained from standard commercial sources, including Shanghai Aladdin Bio-Chem Technology Co., Ltd., Shanghai Macklin Biochemical Co., Ltd., Sigma-Aldrich, Alfa Aesar (China) Chemical Co., Ltd., Tokyo Chemical Industry (Shanghai) Co., Ltd., Energy Chemical Co., Ltd., Shanghai Titan Scientific Co., Ltd., Kelong Chemical Co., Ltd., J&K Scientific and the like.

The technical solutions of the present invention will be described in detail by the following examples, but the scope of protection of the present invention includes but is not limited thereto.

The compounds used in the reactions described herein are prepared according to organic synthesis techniques known to those skilled in the art, and starting from commercially available chemicals and(or) compounds described in chemical documents. “Commercially available chemicals” are obtained from regular commercial sources, and suppliers include: Titan Scientific Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific and the like.

The structures of the compounds are determined by nuclear magnetic resonance (NMR) or (and) mass spectrometry (MS). The NMR shift (δ) is given in the unit of 10⁻⁶ (ppm). NMR is determined with (Bruker Avance III 400 and Bruker Avance 300) NMR instrument; the solvent for determination is deuterated dimethyl sulfoxide (DMSO-d₆), deuterated chloroform (CDCl₃), and deuterated methanol (CD₃OD); and the internal standard is tetramethylsilane (TMS).

MS is determined with Agilent 6120B (ESI) and Agilent 6120B (APCI).

HPLC is determined with Agilent 1260DAD high pressure liquid chromatograph (Zorbax SB-C18 100×4.6 mm, 3.5 μM).

Yantai Huanghai HSGF254 or Qingdao GF254 silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm.

For the column chromatography, Yantai Huanghai silica gel of 200-300 mesh silica gel is generally used as a carrier.

SEM:

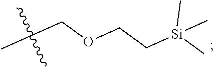 [SMILES: *COCC[Si](C)(C)C]

### THP:

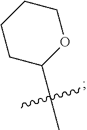 [SMILES: *C1CCCCO1]

Boc: tert-butoxycarbonyl; Ms:

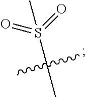 [SMILES: *S(C)(=O)=O]

### TBS:

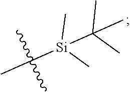 [SMILES: *[Si](C)(C)C(C)(C)C]

MTBE: methyl tert-butyl ether; Bn:

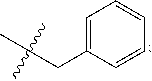 [SMILES: *Cc1ccccc1]

DIPEA: N,N-diisopropylethylamine; DMAc: N,N-dimethylacetamide; DMSO: dimethyl sulfoxide; DCM: dichloromethane; Cbz:

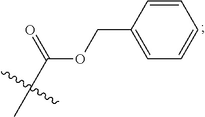 [SMILES: *C(=O)OCc1ccccc1]

NMP: N-methylpyrrolidone; DCE: dichloroethane; THF: tetrahydrofuran; DMF: N,N-dimethylformamide.

### Intermediate 1

### tert-butyl 3-(4-(piperazin-1-yl)piperidin-1-yl)azetidine-1-carboxylate (Intermediate 1)

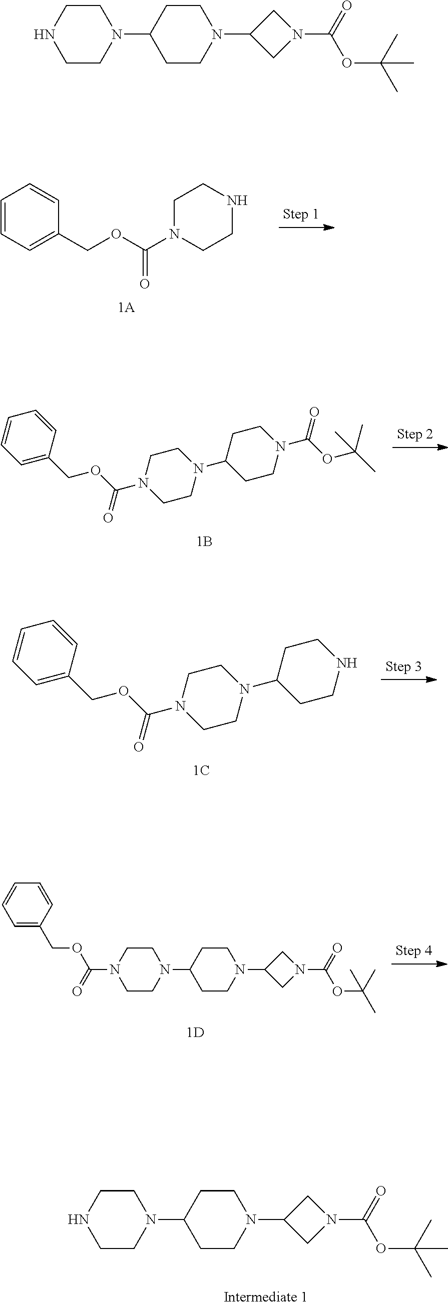 [SMILES: CC(C)(C)OC(=O)N1CC(N2CCC(N3CCNCC3)CC2)C1 ; O=C(OCc1ccccc1)N1CCNCC1 ; CC(C)(C)OC(=O)N1CCC(N2CCN(C(=O)OCc3ccccc3)CC2)CC1 ; O=C(OCc1ccccc1)N1CCN(C2CCNCC2)CC1 ; CC(C)(C)OC(=O)N1CC(N2CCC(N3CCN(C(=O)OCc4ccccc4)CC3)CC2)C1 ; CC(C)(C)OC(=O)N1CC(N2CCC(N3CCNCC3)CC2)C1]

### Step 1: benzyl 4-(1-(tert-butoxycarbonyl)piperidin-4-yl)piperazine-1-carboxylate (1B)

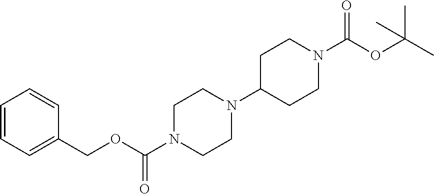 [SMILES: CC(C)(C)OC(=O)N1CCC(N2CCN(C(=O)OCc3ccccc3)CC2)CC1]

Benzyl piperazine-1-carboxylate (2.20 g, 10 mmol) (1A) was dissolved in 25 mL of 1,2-dichloroethane, and tert-butyl 4-oxopiperidine-1-carboxylate (3.0 g, 15 mmol) and 2 mL of acetic acid were successively added. The mixture was stirred at room temperature for 1 h, and then sodium triacetoxyborohydride (6.4 g, 30 mmol) was added. The resulting mixture was stirred at room temperature for 16 h. To the reaction liquid was slowly added 50 mL of saturated sodium bicarbonate solution, and the mixture was extracted with 60 mL of dichloromethane. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=15:1), to afford benzyl 4-(1-(tert-butoxycarbonyl)piperidin-4-yl)piperazine-1-carboxylate (1B) (2.8 g, yield: 69%).

LCMS m/z=404.3 [M+1]⁺

### Step 2: benzyl 4-(piperidin-4-yl)piperazine-1-carboxylate (1C)

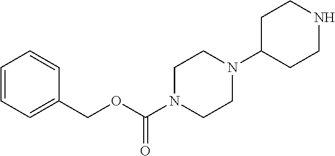 [SMILES: O=C(OCc1ccccc1)N1CCN(C2CCNCC2)CC1]

Benzyl 4-(1-(tert-butoxycarbonyl)piperidin-4-yl)piperazine-1-carboxylate (1B) (2.8 g, 6.94 mmol) was dissolved in 20 mL of dichloromethane, and 20 mL of trifluoroacetic acid was added. The mixture was stirred at room temperature for 1 h. The reaction system was concentrated under reduced pressure, and 50 mL of dichloromethane was added. The mixture was adjusted to pH 9 with a saturated sodium bicarbonate solution, extracted with 50 mL of dichloromethane, dried over anhydrous sodium sulfate, and concentrated under reduced pressure, to afford crude benzyl 4-(piperidin-4-yl)piperazine-1-carboxylate (1C) (1.8 g).

LCMS m/z=304.2 [M+1]⁺

### Step 3: benzyl 4-(1-(1-(tert-butoxycarbonyl)azetidin-3-yl)piperidin-4-yl)piperazine-1-carboxylate (1D)

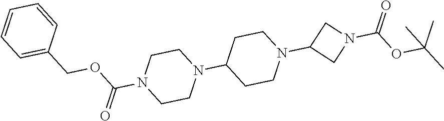 [SMILES: CC(C)(C)OC(=O)N1CC(N2CCC(N3CCN(C(=O)OCc4ccccc4)CC3)CC2)C1]

The above crude benzyl 4-(piperidin-4-yl)piperazine-1-carboxylate (1C) (1.8 g) was dissolved in 20 mL of DCE, and tert-butyl 3-oxoazetidine-1-carboxylate (2.3 g, 13.5 mmol) and 1.5 mL of acetic acid were successively added. The mixture was stirred at room temperature for 60 min, and then sodium triacetoxyborohydride (4.2 g, 19.8 mmol) was added. The resulting mixture was stirred at room temperature for 16 h. To the reaction system was slowly added 50 mL of saturated sodium bicarbonate solution, and the mixture was extracted twice with 60 mL of dichloromethane. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford benzyl 4-(1-(1-(tert-butoxycarbonyl) azetidin-3-yl)piperidin-4-yl)piperazine-1-carboxylate (1D) (2.1 g, two-step total yield from compound 1B: 66%).

LCMS m/z=459.3 [M+1]⁺

### Step 4: tert-butyl 3-(4-(piperazin-1-yl)piperidin-1-yl)azetidine-1-carboxylate (Intermediate 1)

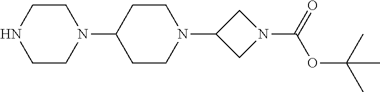 [SMILES: CC(C)(C)OC(=O)N1CC(N2CCC(N3CCNCC3)CC2)C1]

Benzyl 4-(1-(1-(tert-butoxycarbonyl)azetidin-3-yl)piperidin-4-yl)piperazine-1-carboxylate (1D) (1.0 g, 2.18 mmol) was dissolved in 25 mL of methanol, and 0.2 g of 10% palladium on carbon was added. The mixture was stirred at room temperature under hydrogen atmosphere for 20 h. The reaction liquid was filtered and concentrated under reduced pressure, to afford crude tert-butyl 3-(4-(piperazin-1-yl)piperidin-1-yl)azetidine-1-carboxylate (intermediate 1) (0.7 g).

LCMS m/z=325.4 [M+1]⁺

### Intermediate 2

### tert-butyl 4-((4-(piperazin-1-yl)piperidin-1-yl)methyl)piperidine-1-carboxylate (Intermediate 2)

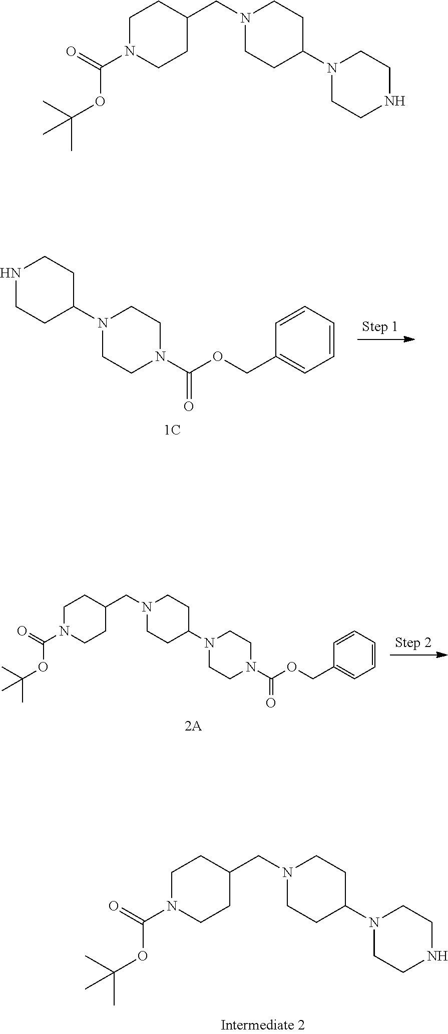 [SMILES: CC(C)(C)OC(=O)N1CCC(CN2CCC(N3CCNCC3)CC2)CC1 ; O=C(OCc1ccccc1)N1CCN(C2CCNCC2)CC1 ; CC(C)(C)OC(=O)N1CCC(CN2CCC(N3CCN(C(=O)OCc4ccccc4)CC3)CC2)CC1 ; CC(C)(C)OC(=O)N1CCC(CN2CCC(N3CCNCC3)CC2)CC1]

### Step 1: benzyl 4-(1-((1-(tert-butoxycarbonyl)piperidin-4-yl)methyl)piperidin-4-yl)piperazine-1-carboxylate (2A)

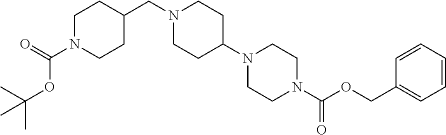 [SMILES: CC(C)(C)OC(=O)N1CCC(CN2CCC(N3CCN(C(=O)OCc4ccccc4)CC3)CC2)CC1]

The above crude benzyl 4-(piperidin-4-yl)piperazine-1-carboxylate (1C) (0.90 g) was dissolved in 15 mL of DCE, and tert-butyl 4-formylpiperidine-1-carboxylate (1.2 g, 5.63 mmol) and 1 mL of acetic acid were successively added. The mixture was stirred at room temperature for 60 min, and then sodium triacetoxyborohydride (2.1 g, 9.9 mmol) was added. The resulting mixture was reacted at room temperature for 16 h. To the reaction system was slowly added 50 mL of saturated sodium bicarbonate solution, and the mixture was extracted twice with 60 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford benzyl 4-(1-((1-(tert-butoxycarbonyl) piperidin-4-yl)methyl)piperidin-4-yl)piperazine-1-carboxylate (2A) (1.1 g, two-step yield from compound 1B: 63%).

LCMS m/z=501.3 [M+1]⁺

### Step 2: tert-butyl 4-((4-(piperazin-1-yl)piperidin-1-yl)methyl)piperidine-1-carboxylate (Intermediate 2)

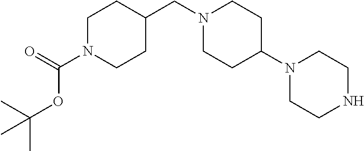 [SMILES: CC(C)(C)OC(=O)N1CCC(CN2CCC(N3CCNCC3)CC2)CC1]

Benzyl 4-(1-((1-(tert-butoxycarbonyl)piperidin-4-yl)methyl)piperidin-4-yl)piperazine-1-carboxylate (2A) (1.1 g, 2.2 mmol) was dissolved in 25 mL of methanol, and 0.2 g of 10% palladium on carbon was added. The mixture was reacted at room temperature under hydrogen atmosphere for 20 h. The reaction system was filtered by suction, and the filtrate was concentrated under reduced pressure, to afford crude tert-butyl 4-((4-(piperazin-1-yl)piperidin-1-yl)methyl)piperidine-1-carboxylate (intermediate 2) (0.62 g).

LCMS m/z=367.2 [M+1]⁺

### Example 1

### 5-chloro-N-(2-chloro-4-(trifluoromethyl)phenyl)-2-{[(4-[(4-(2-(2,6-dioxopiperidin-3-yl)-1,3-dioxo-2,3-dihydro-1H-isoindol-5-yl)piperazin-1-yl)methyl]piperidin-1-yl)sulfonyl]amino}benzamide (Compound 1)

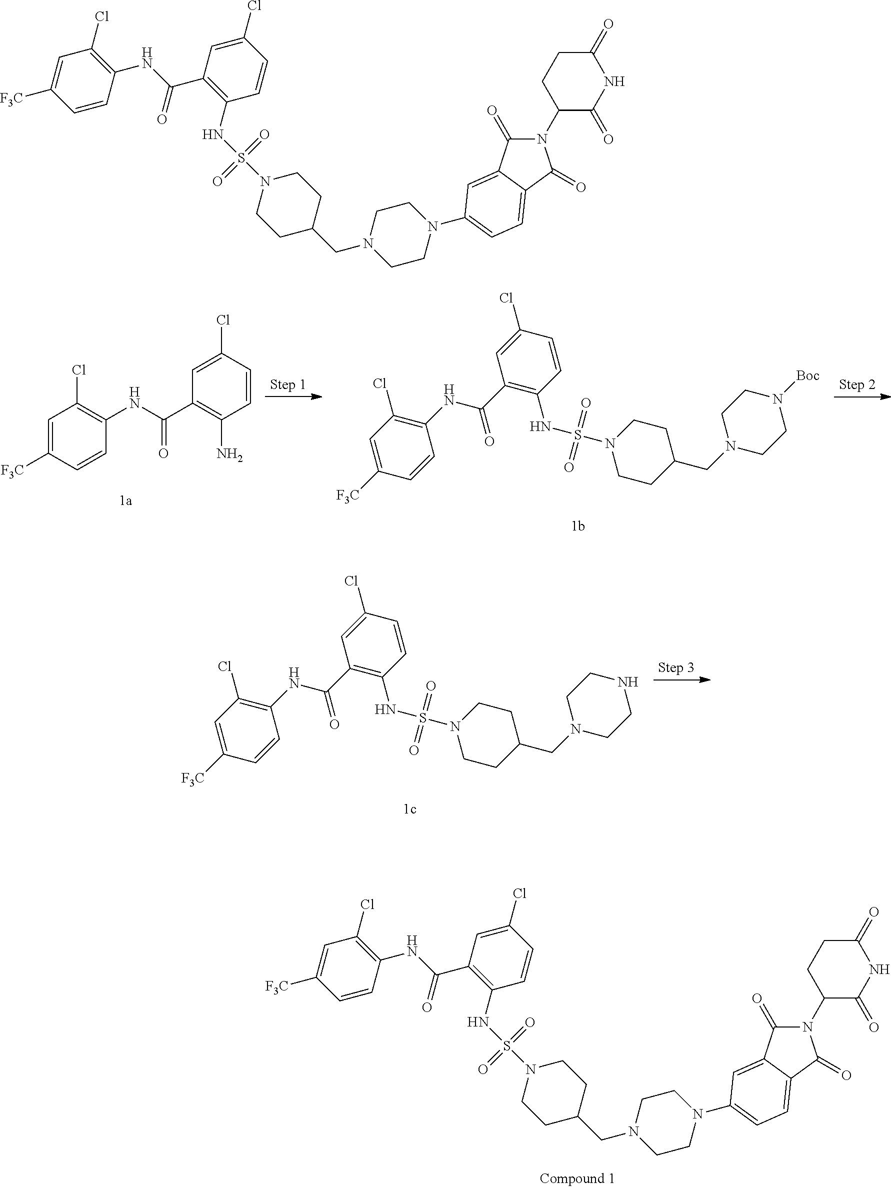 [SMILES: O=C1CCC(N2C(=O)c3ccc(N4CCN(CC5CCN(S(=O)(=O)Nc6ccc(Cl)cc6C(=O)Nc6ccc(C(F)(F)F)cc6Cl)CC5)CC4)cc3C2=O)C(=O)N1 ; Nc1ccc(Cl)cc1C(=O)Nc1ccc(C(F)(F)F)cc1Cl ; CC(C)(C)OC(=O)N1CCN(CC2CCN(S(=O)(=O)Nc3ccc(Cl)cc3C(=O)Nc3ccc(C(F)(F)F)cc3Cl)CC2)CC1 ; O=C(Nc1ccc(C(F)(F)F)cc1Cl)c1cc(Cl)ccc1NS(=O)(=O)N1CCC(CN2CCNCC2)CC1 ; O=C1CCC(N2C(=O)c3ccc(N4CCN(CC5CCN(S(=O)(=O)Nc6ccc(Cl)cc6C(=O)Nc6ccc(C(F)(F)F)cc6Cl)CC5)CC4)cc3C2=O)C(=O)N1]

Step 1: tert-butyl 4-[1-[(4-chloro-2-[(2-chloro-4-(trifluoromethyl)phenyl)carbamoyl]phenyl)sulfamoyl]piperidin-4-yl)methyl]piperazine-1-carboxylate (1b)

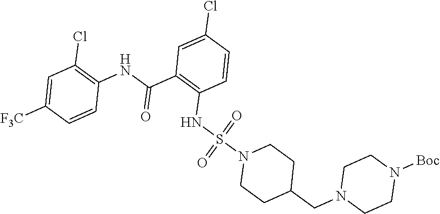 [SMILES: CC(C)(C)OC(=O)N1CCN(CC2CCN(S(=O)(=O)Nc3ccc(Cl)cc3C(=O)Nc3ccc(C(F)(F)F)cc3Cl)CC2)CC1]

1,1′-sulfonyldiimidazole (4.0 g, 20.2 mmol) was dissolved in 50 mL of dichloromethane, and the mixture was cooled to 0° C. under nitrogen protection. Methyl trifluoromethanesulfonate (3.65 g, 22.2 mmol) was slowly added, and after the addition, the mixture was warmed to room temperature and reacted for 3 h. The reaction liquid was concentrated under reduced pressure, and the residue was dissolved with 50 mL of acetonitrile. Tert-butyl 4-(piperidin-4-ylmethyl)piperazine-1-carboxylate (4.0 g, 14.1 mmol) (see WO 2019195609 for the synthetic method) was added, and the mixture was reacted at room temperature for 18 h. The reaction liquid was concentrated under reduced pressure, and to the residue were added 200 mL of dichloromethane and 50 mL of water. Liquid separation was performed. The organic layer was washed with 50 mL of saturated sodium chloride, dried over anhydrous sodium sulfate, and concentrated under reduced pressure. The resulting crude was then separated and purified by silica gel column chromatography (petroleum ether/ethyl acetate (v/v)=4:1-1:4), to afford 2.4 g of intermediate. The intermediate (0.6 g) was dissolved in 10 mL of dichloromethane, and the mixture was cooled to 0° C. under nitrogen protection. Methyl trifluoromethanesulfonate (0.26 g, 1.58 mmol) was added, and after the addition, the mixture was warmed to room temperature and reacted for 3 h. The reaction liquid was concentrated under reduced pressure, and the residue was dissolved with 10 mL of acetonitrile. 2-amino-5-chloro-N-(2-chloro-4-(trifluoromethyl)phenyl)benzamide (1a) (0.350 g, 1.00 mmol) (see WO 2019179436 for the synthetic method) was added, and the reaction was warmed to 90° C. and stirred for 18 h. The reaction liquid was cooled to room temperature, and concentrated under reduced pressure. The crude product was then separated and purified by silica gel column chromatography (petroleum ether/ethyl acetate (v/v)=3:2), to afford 1b (0.1 g, yield from compound 1a: 14%).

LCMS m/z=694.1 [M+1]⁺

### Step 2: 5-chloro-N-(2-chloro-4-(trifluoromethyl)phenyl)-2-{[(4-[(piperazin-1-yl)methyl]piperidin-1-yl)sulfonyl]amino}benzamide (1c)

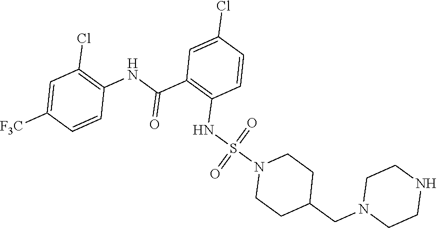 [SMILES: O=C(Nc1ccc(C(F)(F)F)cc1Cl)c1cc(Cl)ccc1NS(=O)(=O)N1CCC(CN2CCNCC2)CC1]

1b (0.1 g, 0.144 mmol) was dissolved in 4 mL of dichloromethane, and 2 mL of trifluoroacetic acid was added. After the addition, the mixture was stirred at room temperature for 2 h. The reaction liquid was concentrated under reduced pressure, and to the residue was added 20 mL of dichloromethane. The mixture was adjusted to pH 8 with 2 mol/L sodium hydroxide solution. Liquid separation was performed. The aqueous layer was extracted with 10 mL of dichloromethane, and the organic layers were combined, dried over anhydrous sodium sulfate, and concentrated under reduced pressure, to afford crude 1c (0.08 g).

### Step 3: 5-chloro-N-(2-chloro-4-(trifluoromethyl)phenyl)-2-{[(4-[(4-(2-(2,6-dioxopiperidin-3-yl)-1,3-dioxo-2,3-dihydro-1H-isoindol-5-yl)piperazin-1-yl)methyl]piperidin-1-yl)sulfonyl]amino}benzamide (Compound 1)

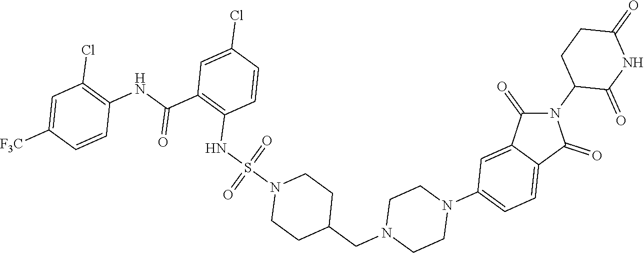 [SMILES: O=C1CCC(N2C(=O)c3ccc(N4CCN(CC5CCN(S(=O)(=O)Nc6ccc(Cl)cc6C(=O)Nc6ccc(C(F)(F)F)cc6Cl)CC5)CC4)cc3C2=O)C(=O)N1]

The above crude 1c (0.08 g) was dissolved in 2 mL of DMSO, and diisopropylethylamine (0.09 g, 0.7 mmol) and 2-(2,6-dioxopiperidin-3-yl)-5-fluoroisoindoline-1,3-dione (see WO 2017197056 for the synthetic method) (0.058 g, 0.21 mmol) were added. The mixture was heated to 90° C. and reacted for 7 h. The reaction liquid was cooled to room temperature, and 20 mL of water and 20 mL of ethyl acetate were added. Liquid separation was performed. The organic layer was washed with a saturated sodium chloride solution (20 mL×3), dried over anhydrous sodium sulfate, and concentrated under reduced pressure. The crude product was then separated and purified by silica gel column chromatography (ethyl acetate), to afford compound 1 (0.036 g, two-step yield from compound 1b: 29%).

¹H NMR (400 MHz, CDCl₃) δ 10.06 (s, 1H), 8.57 (d, 1H), 8.49 (s, 1H), 7.99 (s, 1H), 7.76-7.59 (m, 5H), 7.51 (dd, 1H), 7.29-7.24 (m, 1H), 7.04 (dd, 1H), 4.93 (dd, 1H), 3.89-3.78 (m, 2H), 3.51-3.36 (m, 4H), 2.95-2.67 (m, 5H), 2.67-2.48 (m, 4H), 2.34-2.23 (m, 2H), 2.17-2.07 (m, 1H), 1.92-1.81 (m, 2H), 1.77-1.61 (m, 1H), 0.92-0.81 (m, 2H).

LCMS m/z=850.2 [M+1]⁺

### Example 2

### N-(4-chloro-2-(7-chloro-5-(trifluoromethyl)-1H-1,3-benzodiazol-2-yl)phenyl)-4-(1-(2-(2,6-dioxopiperidin-3-yl)-1,3-dioxo-2,3-dihydro-1H-isoindol-5-yl)azetidin-3-yl)piperazine-1-sulfonamide (Compound 2)

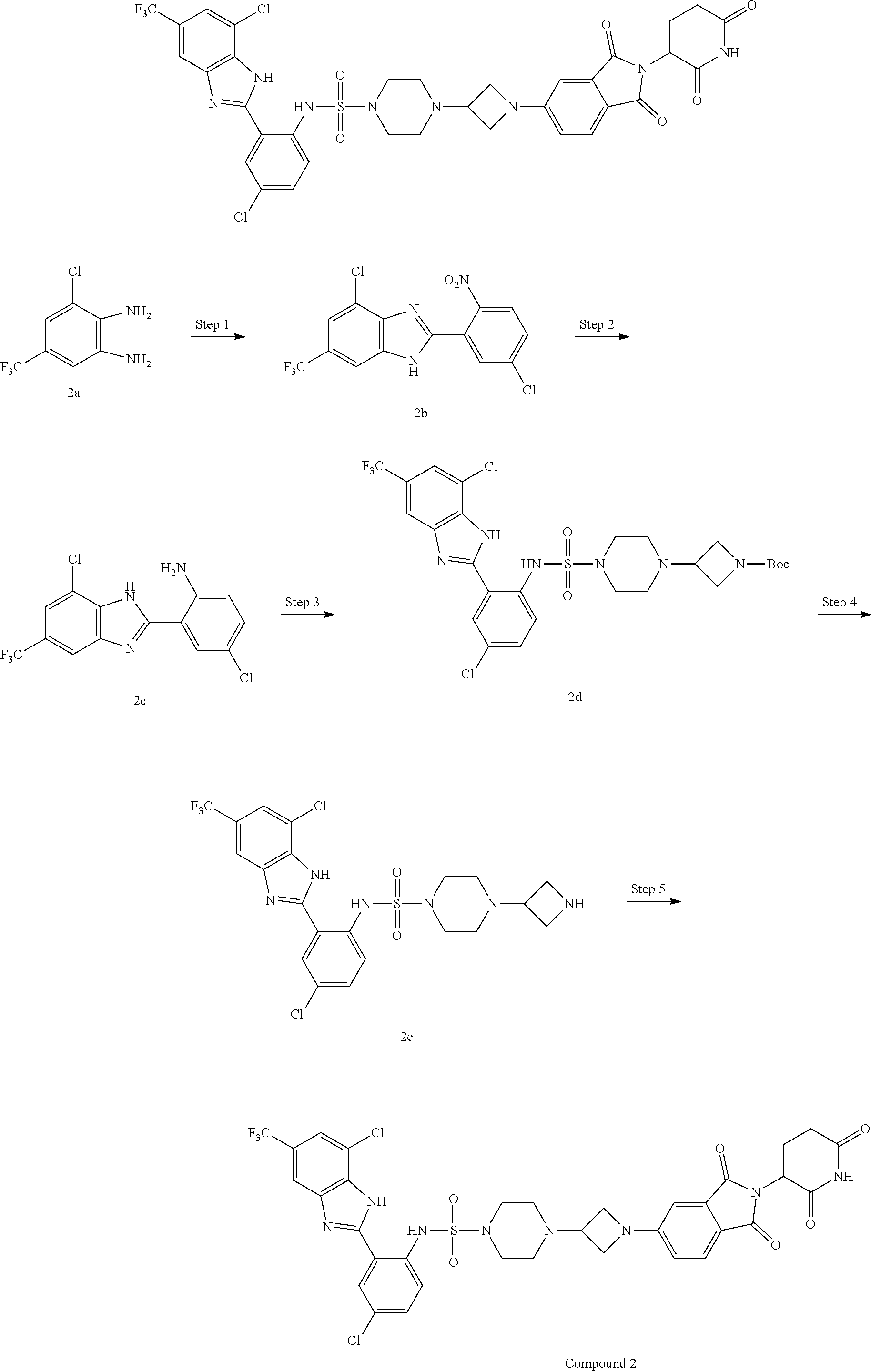

### Step 1: 7-chloro-2-(5-chloro-2-nitrophenyl)-5-(trifluoromethyl)-1H-1,3-benzodiazole (2b)

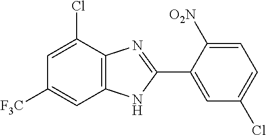 [SMILES: O=[N+]([O-])c1ccc(Cl)cc1-c1nc2c(Cl)cc(C(F)(F)F)cc2[nH]1]

5-chloro-2-nitrobenzoic acid (4.79 g, 23.76 mmol) was suspended in 90 mL of phosphorus oxychloride, and 3-chloro-5-(trifluoromethyl)benzene-1,2-diamine (2a) (5.00 g, 23.74 mmol) was added. After the addition, the mixture was reacted at 100° C. for 18 h. The reaction liquid was cooled to room temperature, slowly added dropwise to 100 mL of water cooled in an ice bath, and filtered. The filtrate was extracted with 100 mL of ethyl acetate, and the filter cake was dissolved with 200 mL of ethyl acetate. The ethyl acetate solutions were combined, washed with 100 mL of saturated sodium chloride, dried over anhydrous sodium sulfate, and concentrated under reduced pressure, to afford crude 2b (8.5 g).

### Step 2: 4-chloro-2-(7-chloro-5-(trifluoromethyl)-1H-1,3-benzodiazol-2-yl)aniline (2c)

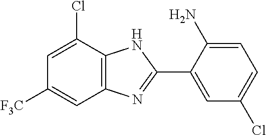 [SMILES: Nc1ccc(Cl)cc1-c1nc2cc(C(F)(F)F)cc(Cl)c2[nH]1]

The above crude 2b (8.5 g) was dissolved in 160 mL of anhydrous ethanol, and 40 mL of water and solid ammonium chloride (6.04 g, 112.9 mmol) were successively added, and then iron powder (6.31 g, 112.7 mmol) was added. After the addition, the mixture was warmed to 90° C. and reacted for 2 h. The reaction liquid was cooled to room temperature and filtered through celite. To the filtrate were added 200 mL of water and 500 mL of ethyl acetate. Liquid separation was performed. The organic layer was washed with saturated sodium chloride (200 mL×3), dried over anhydrous sodium sulfate, and concentrated under reduced pressure. The resulting crude was then separated and purified by silica gel column chromatography (petroleum ether/ethyl acetate (v/v)=20:1), to afford 2c (3.70 g, two-step total yield from compound 2a: 45%).

LCMS m/z=346.0 [M+1]⁺

### Step 3: tert-butyl 3-(4-[(4-chloro-2-(7-chloro-5-(trifluoromethyl)-1H-1,3-benzodiazol-2-yl)phenyl) sulfamoyl]piperazin-1-yl)azetidine-1-carboxylate (2d)

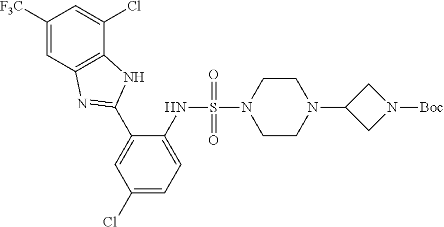 [SMILES: CC(C)(C)OC(=O)N1CC(N2CCN(S(=O)(=O)Nc3ccc(Cl)cc3-c3nc4cc(C(F)(F)F)cc(Cl)c4[nH]3)CC2)C1]

1,1′-sulfonyldiimidazole (0.105 g, 0.53 mmol) was dissolved in 10 mL of dichloromethane, and the mixture was cooled to 0° C. under nitrogen protection. Methyl trifluoromethanesulfonate (0.095 g, 0.58 mmol) was added, and after the addition, the mixture was warmed to room temperature and reacted for 3 h. The reaction liquid was concentrated under reduced pressure, and the residue was dissolved with 10 mL of acetonitrile. Tert-butyl 3-(piperazin-1-yl)azetidine-1-carboxylate (see WO 2019195609 for the synthetic method) (0.200 g, 0.83 mmol) was added, and the mixture was reacted at room temperature for 18 h. The reaction liquid was concentrated under reduced pressure, and 50 mL of dichloromethane and 30 mL of water were added. Liquid separation was performed. The aqueous layer was extracted with dichloromethane (20 mL×2), and the organic layers were combined, washed with 20 mL of saturated sodium chloride, dried over anhydrous sodium sulfate, and concentrated under reduced pressure. The crude product was dissolved in 10 mL of dichloromethane, and the mixture was cooled to 0° C. under nitrogen protection. Methyl trifluoromethanesulfonate (0.095 g, 0.58 mmol) was added, and after the addition, the mixture was warmed to room temperature and reacted for 3 h. The reaction liquid was concentrated under reduced pressure, and the residue was dissolved with 10 mL of acetonitrile. 2c (0.18 g, 0.52 mmol) was added, and the mixture was reacted at 90° C. for 18 h. The reaction liquid was concentrated under reduced pressure, and to the residue were added 30 mL of dichloromethane and 20 mL of water. Liquid separation was performed. The aqueous layer was extracted with 20 mL of dichloromethane, and the organic layers were combined, washed with 20 mL of saturated sodium chloride solution, dried over anhydrous sodium sulfate, and concentrated under reduced pressure. The crude product was then separated and purified by silica gel column chromatography (petroleum ether/ethyl acetate (v/v)=3:2), to afford 2d (0.16 g, yield from compound 2c: 47%).

### Step 4: 4-(azetidin-3-yl)-N-(4-chloro-2-(7-chloro-5-(trifluoromethyl)-1H-1,3-benzodiazol-2-yl)phenyl)piperazine-1-sulfonamide (2e)

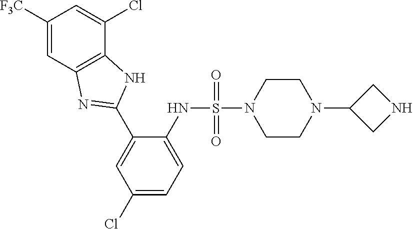 [SMILES: O=S(=O)(Nc1ccc(Cl)cc1-c1nc2cc(C(F)(F)F)cc(Cl)c2[nH]1)N1CCN(C2CNC2)CC1]

2d (0.15 g, 0.23 mmol) was dissolved in 4 mL of dichloromethane, and 2 mL of trifluoroacetic acid was added. After the addition, the mixture was stirred at room temperature for 2 h. The reaction liquid was concentrated under reduced pressure, and to the residue was added 20 mL of dichloromethane. The mixture was adjusted to pH 8 with 2 mol/L sodium hydroxide solution. Liquid separation was performed. The aqueous layer was extracted with 10 mL of dichloromethane, and the organic layers were combined, dried over anhydrous sodium sulfate, and concentrated under reduced pressure, to afford crude 2e (0.12 g).

LCMS m/z=549.1 [M+1]⁺

### Step 5: N-(4-chloro-2-(7-chloro-5-(trifluoromethyl)-1H-1,3-benzodiazol-2-yl)phenyl)-4-(1-(2-(2,6-dioxopiperidin-3-yl)-1,3-dioxo-2,3-dihydro-1H-isoindol-5-yl)azetidin-3-yl)piperazine-1-sulfonamide (Compound 2)

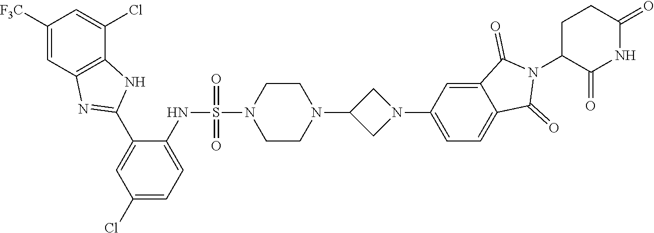 [SMILES: O=C1CCC(N2C(=O)c3ccc(N4CC(N5CCN(S(=O)(=O)Nc6ccc(Cl)cc6-c6nc7cc(C(F)(F)F)cc(Cl)c7[nH]6)CC5)C4)cc3C2=O)C(=O)N1]

The above crude 2e (0.12 g) was dissolved in 2 mL of DMSO, and diisopropylethylamine (0.140 g, 1.08 mmol) and 2-(2,6-dioxopiperidin-3-yl)-5-fluoroisoindoline-1,3-dione (see WO 2017197056 for the synthetic method) (0.091 g, 0.330 mmol) were successively added. After the addition, the mixture was heated to 90° C. and reacted for 3 h. The reaction liquid was cooled to room temperature, and 20 mL of water and 20 mL of ethyl acetate were added. Liquid separation was performed. The organic layer was washed with saturated sodium chloride (20 mL×3), dried over anhydrous sodium sulfate, and concentrated under reduced pressure. The resulting crude was then separated and purified by silica gel column chromatography (ethyl acetate), to afford compound 2 (0.08 g, two-step total yield from compound 2d: 43%).

¹H NMR (400 MHz, DMSO-d₆) δ 14.05 (br.s, 1H), 12.50 (br.s, 1H), 11.04 (s, 1H), 8.35 (br.s, 1H), 7.93 (br.s, 1H), 7.82-7.72 (m, 2H), 7.68-7.58 (m, 2H), 6.72 (d, 1H), 6.58 (dd, 1H), 5.04 (dd, 1H), 4.08-3.97 (m, 2H), 3.81-3.70 (m, 2H), 3.39-3.31 (m, 1H), 3.26-3.12 (m, 4H), 2.94-2.79 (m, 1H), 2.64-2.44 (m, 2H), 2.41-2.28 (m, 4H), 2.05-1.93 (m, 1H).

LCMS m/z=805.1 [M+1]⁺

### Example 3

### 5-chloro-N-(2-chloro-4-(trifluoromethyl)phenyl)-2-((4-((1-(2-(2,6-dioxopiperidin-3-yl)-1,3-dioxoisoindolin-5-yl)piperidin-4-yl)methyl)piperazine)-1-sulfonamido)benzamide (Compound 3)

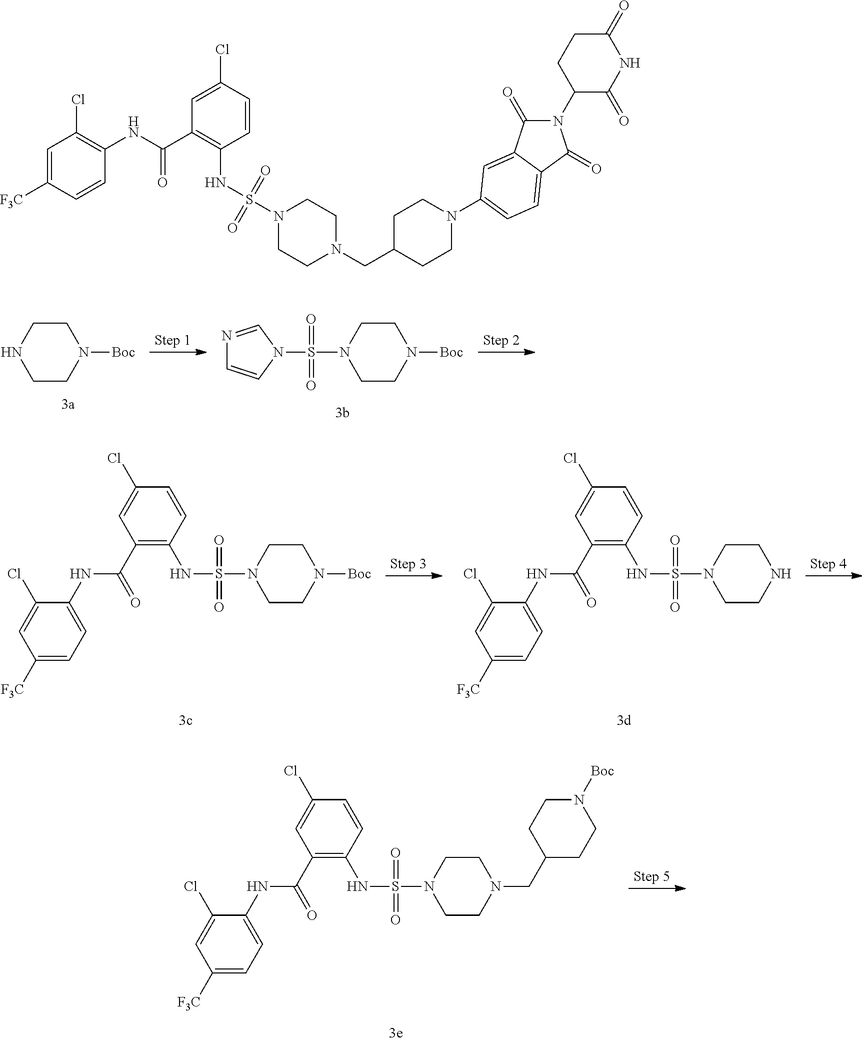 [SMILES: O=C1CCC(N2C(=O)c3ccc(N4CCC(CN5CCN(S(=O)(=O)Nc6ccc(Cl)cc6C(=O)Nc6ccc(C(F)(F)F)cc6Cl)CC5)CC4)cc3C2=O)C(=O)N1 ; CC(C)(C)OC(=O)N1CCNCC1 ; CC(C)(C)OC(=O)N1CCN(S(=O)(=O)n2ccnc2)CC1 ; CC(C)(C)OC(=O)N1CCN(S(=O)(=O)Nc2ccc(Cl)cc2C(=O)Nc2ccc(C(F)(F)F)cc2Cl)CC1 ; O=C(Nc1ccc(C(F)(F)F)cc1Cl)c1cc(Cl)ccc1NS(=O)(=O)N1CCNCC1 ; CC(C)(C)OC(=O)N1CCC(CN2CCN(S(=O)(=O)Nc3ccc(Cl)cc3C(=O)Nc3ccc(C(F)(F)F)cc3Cl)CC2)CC1]

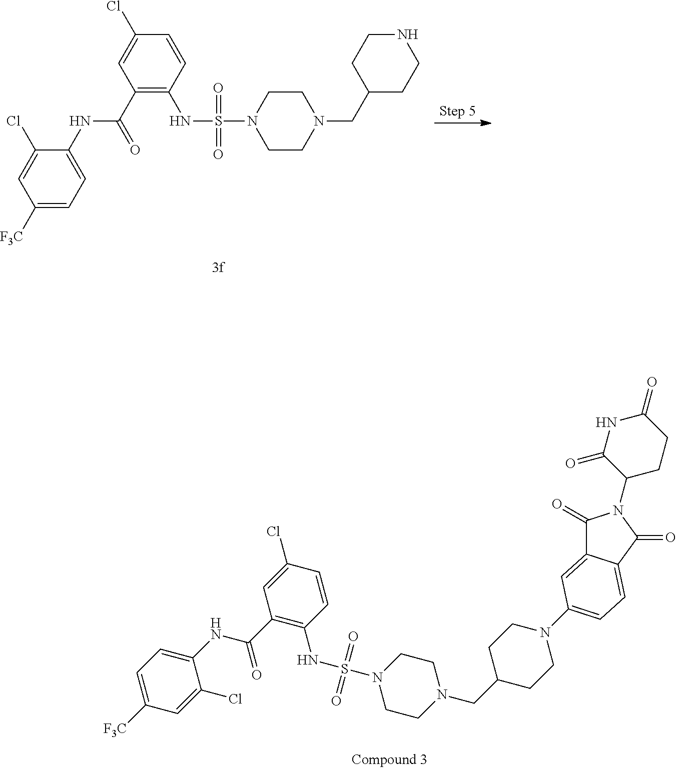 [SMILES: O=C(Nc1ccc(C(F)(F)F)cc1Cl)c1cc(Cl)ccc1NS(=O)(=O)N1CCN(CC2CCNCC2)CC1 ; O=C1CCC(N2C(=O)c3ccc(N4CCC(CN5CCN(S(=O)(=O)Nc6ccc(Cl)cc6C(=O)Nc6ccc(C(F)(F)F)cc6Cl)CC5)CC4)cc3C2=O)C(=O)N1]

### Step 1: tert-butyl 4-((1H-imidazol-1-yl)sulfonyl)piperazine-1-carboxylate (3b)

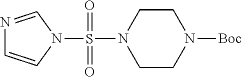 [SMILES: CC(C)(C)OC(=O)N1CCN(S(=O)(=O)n2ccnc2)CC1]

1,1′-sulfonyldiimidazole (5.00 g, 25.23 mmol) was dissolved in 15 mL of DCM, and methyl trifluoromethanesulfonate (4.55 g, 27.73 mmol) was added under an ice bath. The mixture was warmed to room temperature and reacted with stirring for 3 h. The reaction liquid was concentrated under reduced pressure, and the residue was dissolved in 20 mL of acetonitrile. Tert-butyl piperazine-1-carboxylate (3a) (4.70 g, 25.23 mmol) was added, and the mixture was reacted at room temperature for 16 h. The reaction liquid was concentrated under reduced pressure, and the crude product was separated and purified by silica gel column chromatography (petroleum ether/ethyl acetate (v/v)=10:1-4:1), to afford 3b (3.5 g, yield: 44%).

### Step 2: tert-butyl 4-(N-(4-chloro-2-((2-chloro-4-(trifluoromethyl)phenyl)carbamoyl)phenyl) sulfamoyl) piperazine-1-carboxylate (3c)

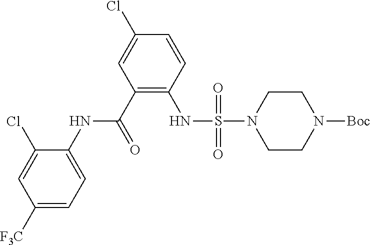 [SMILES: CC(C)(C)OC(=O)N1CCN(S(=O)(=O)Nc2ccc(Cl)cc2C(=O)Nc2ccc(C(F)(F)F)cc2Cl)CC1]

3b (700 mg, 2.21 mmol) was dissolved in 8 mL of DCM, and methyl trifluoromethanesulfonate (798 mg, 4.86 mmol) was added under an ice bath. The mixture was reacted at room temperature for 3 h. The reaction liquid was concentrated under reduced pressure, and the residue was dissolved in 15 mL of acetonitrile. 2-amino-5-chloro-N-(2-chloro-4-(trifluoromethyl)phenyl)benzamide (1a) (0.350 g, 1.00 mmol) (384 mg, 1.10 mmol) (see WO 2019179436 for the synthetic method) was then added, and the mixture was refluxed and reacted for 16 h. The reaction liquid was cooled to room temperature, and concentrated under reduced pressure. The crude product was then separated and purified by silica gel column chromatography (petroleum ether/ethyl acetate (v/v)=8:1-2:1), to afford 3c (180 mg, yield from compound 1a: 27%).

### Step 3: 5-chloro-N-(2-chloro-4-(trifluoromethyl)phenyl)-2-(piperazine-1-sulfonamido)benzamide (3d)

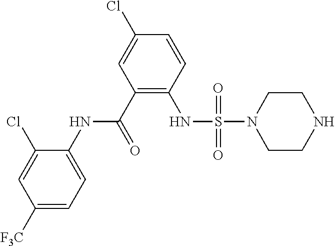 [SMILES: O=C(Nc1ccc(C(F)(F)F)cc1Cl)c1cc(Cl)ccc1NS(=O)(=O)N1CCNCC1]

3c (180 mg, 0.30 mmol) was dissolved in 3 mL of dichloromethane, and 1 mL of trifluoroacetic acid was added. The mixture was reacted at room temperature for 2.5 h. The reaction liquid was adjusted to pH 10 with 2 mol/L aqueous sodium hydroxide solution. The aqueous phase was extracted with dichloromethane (10 mL×3), and the organic phases were combined, dried over anhydrous sodium sulfate, and concentrated under reduced pressure, to afford 3d (150 mg, yield: >99%).

### Step 4: tert-butyl 4-((4-(N-(4-chloro-2-((2-chloro-4-(trifluoromethyl)phenyl)carbamoyl)phenyl) sulfamoyl)piperazin-1-yl)methyl)piperidine-1-carboxylate (3e)

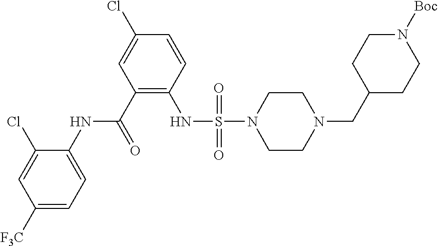 [SMILES: CC(C)(C)OC(=O)N1CCC(CN2CCN(S(=O)(=O)Nc3ccc(Cl)cc3C(=O)Nc3ccc(C(F)(F)F)cc3Cl)CC2)CC1]

3d (150 mg, 0.30 mmol) was dissolved in 5 mL of DCE, and N-Boc-piperidine-4-carbaldehyde (128 mg, 0.60 mmol), glacial acetic acid (0.10 mL, 1.75 mmol) and anhydrous sodium sulfate (200 mg) were successively added at room temperature. The mixture was stirred for 30 min, and then sodium triacetoxyborohydride (213 mg, 1.01 mmol) was added. The resulting mixture was stirred at room temperature for 16 h. To the reaction liquid was added 20 mL of water, and the mixture was adjusted to pH 10 with a saturated aqueous sodium bicarbonate solution, and extracted with dichloromethane (30 mL×3). The organic phases were combined, washed with water (20 mL×2), dried over anhydrous sodium sulfate, and concentrated under reduced pressure. The crude product was separated and purified by silica gel column chromatography (dichloromethane/methanol (v/v)=10:1-8:1), to afford 3e (146 mg, yield: 70%).

### Step 5: 5-chloro-N-(2-chloro-4-(trifluoromethyl)phenyl)-2-((4-(piperidin-4-ylmethyl) piperazine)-1-sulfonamido)benzamide (3f)

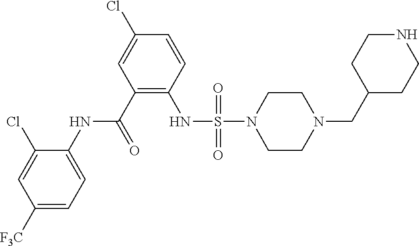 [SMILES: O=C(Nc1ccc(C(F)(F)F)cc1Cl)c1cc(Cl)ccc1NS(=O)(=O)N1CCN(CC2CCNCC2)CC1]

3e (146 mg, 0.21 mmol) was dissolved in 3 mL of DCM, and 1 mL of 2 mol/L hydrochloric acid 1,4-dioxane solution was added. The mixture was reacted at room temperature for 2.5 h. The reaction liquid was adjusted to pH 7 with a saturated aqueous sodium bicarbonate solution. The aqueous phase was extracted with dichloromethane (10 mL×3), and the organic phases were combined, dried over anhydrous sodium sulfate, and concentrated under reduced pressure, to afford crude 3f (124 mg).

### Step 6: 5-chloro-N-(2-chloro-4-(trifluoromethyl)phenyl)-2-((4-((1-(2-(2,6-dioxopiperidin-3-yl)-1,3-dioxoisoindolin-5-yl)piperidin-4-yl)methyl)piperazine)-1-sulfonamido)benzamide (Compound 3)

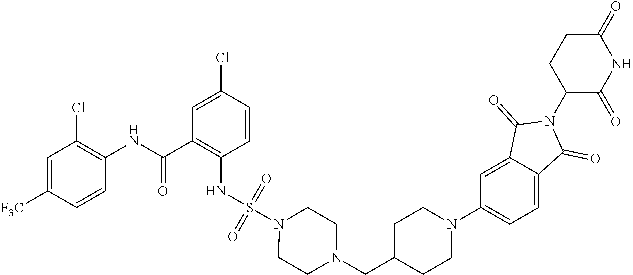 [SMILES: O=C1CCC(N2C(=O)c3ccc(N4CCC(CN5CCN(S(=O)(=O)Nc6ccc(Cl)cc6C(=O)Nc6ccc(C(F)(F)F)cc6Cl)CC5)CC4)cc3C2=O)C(=O)N1]

The above crude 3f (124 mg) was dissolved in 8 mL of DMSO, and 2-(2,6-dioxopiperidin-3-yl)-5-fluoroisoindoline-1,3-dione (see WO 2017197056 for the synthetic method) (69 mg, 0.25 mmol) and diisopropylethylamine (140 mg, 1.08 mmol) were added at room temperature. The mixture was warmed to 80° C. and reacted for 3.5 h. The reaction liquid was cooled to room temperature, and to the reaction liquid was poured 20 mL of water. The aqueous phase was extracted with dichloromethane/methanol (v/v)=10:1 (30 mL×3), and the organic phases were combined, washed with 50 mL of water, dried over anhydrous sodium sulfate, and concentrated under reduced pressure. The crude product was then separated and purified by silica gel column chromatography (dichloromethane/methanol (v/v)=15:1-8:1), to afford compound 3 (18 mg, two-step total yield from compound 3e: 10%).

¹H NMR (400 MHz, DMSO-d₆) δ 10.10 (br.s, 1H), 8.59-8.47 (m, 2H), 8.24 (s, 1H), 7.78-7.71 (m, 2H), 7.68-7.58 (m, 3H), 7.51 (dd, 1H), 7.26-7.24 (m, 1H), 7.01 (dd, 1H), 4.92 (dd, 1H), 3.98-3.86 (m, 2H), 3.38-3.22 (m, 4H), 3.00-2.64 (m, 5H), 2.54-2.37 (m, 4H), 2.27-2.05 (m, 3H), 1.89-1.68 (m, 3H), 1.35-1.15 (m, 2H).

LCMS m/z=850.2 [M+1]⁺

### Example 4

### 3-chloro-2-(2-chloroethoxy)-5-(2-(4-((2-(4-(1′-(2-(2,6-dioxopiperidin-3-yl)-1,3-dioxoisoindolin-5-yl)-[1,3′-biazetidin]-3-yl)piperazin-1-yl)pyrimidin-4-yl)methoxy)phenyl)propan-2-yl)benzonitrile (Compound 4)

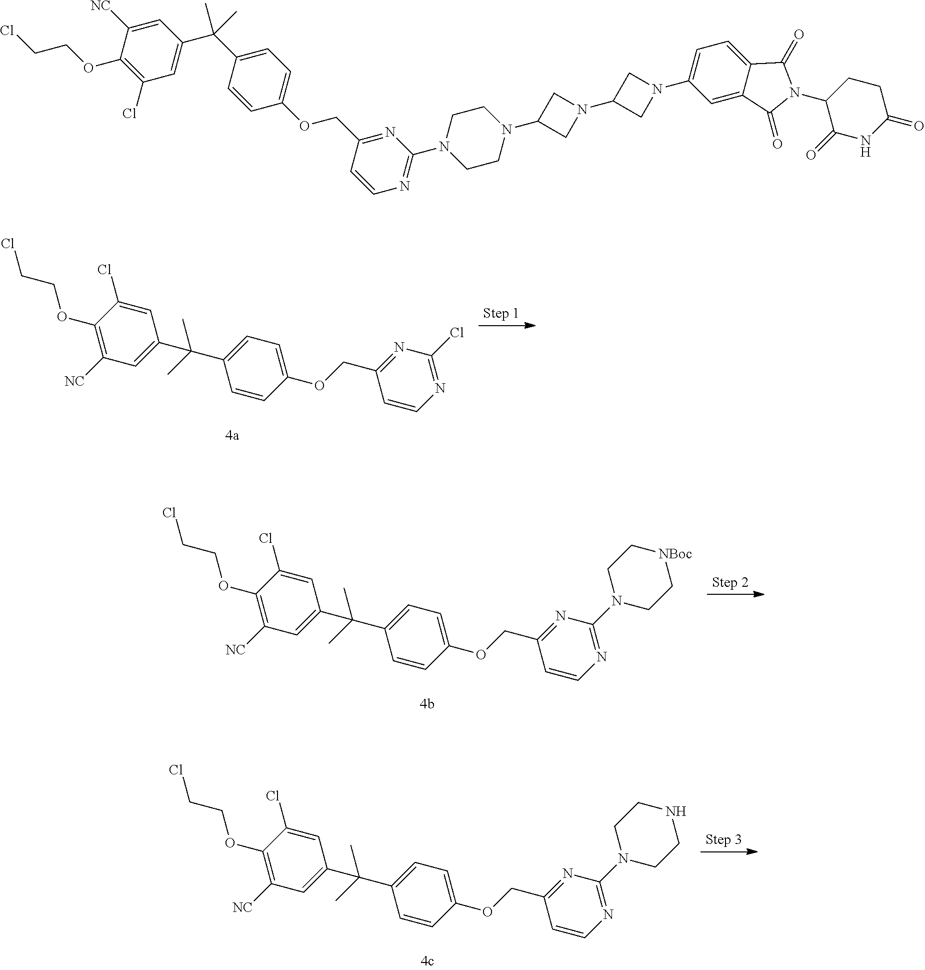 [SMILES: CC(C)(c1ccc(OCc2ccnc(N3CCN(C4CN(C5CN(c6ccc7c(c6)C(=O)N(C6CCC(=O)NC6=O)C7=O)C5)C4)CC3)n2)cc1)c1cc(Cl)c(OCCCl)c(C#N)c1 ; CC(C)(c1ccc(OCc2ccnc(Cl)n2)cc1)c1cc(Cl)c(OCCCl)c(C#N)c1 ; CC(C)(C)OC(=O)N1CCN(c2nccc(COc3ccc(C(C)(C)c4cc(Cl)c(OCCCl)c(C#N)c4)cc3)n2)CC1 ; CC(C)(c1ccc(OCc2ccnc(N3CCNCC3)n2)cc1)c1cc(Cl)c(OCCCl)c(C#N)c1]

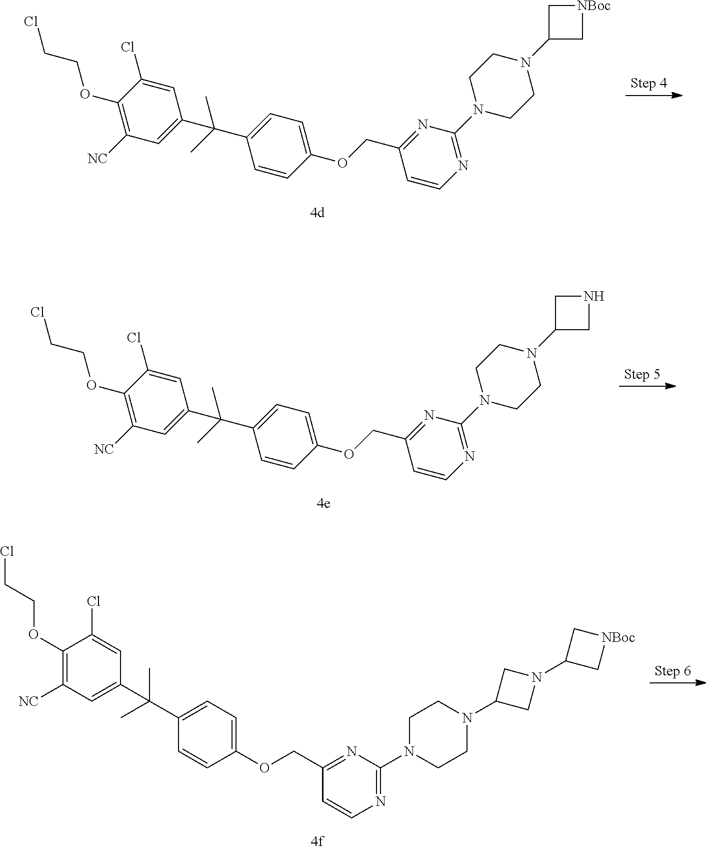 [SMILES: CC(C)(C)OC(=O)N1CC(N2CCN(c3nccc(COc4ccc(C(C)(C)c5cc(Cl)c(OCCCl)c(C#N)c5)cc4)n3)CC2)C1 ; CC(C)(c1ccc(OCc2ccnc(N3CCN(C4CNC4)CC3)n2)cc1)c1cc(Cl)c(OCCCl)c(C#N)c1 ; CC(C)(C)OC(=O)N1CC(N2CC(N3CCN(c4nccc(COc5ccc(C(C)(C)c6cc(Cl)c(OCCCl)c(C#N)c6)cc5)n4)CC3)C2)C1]

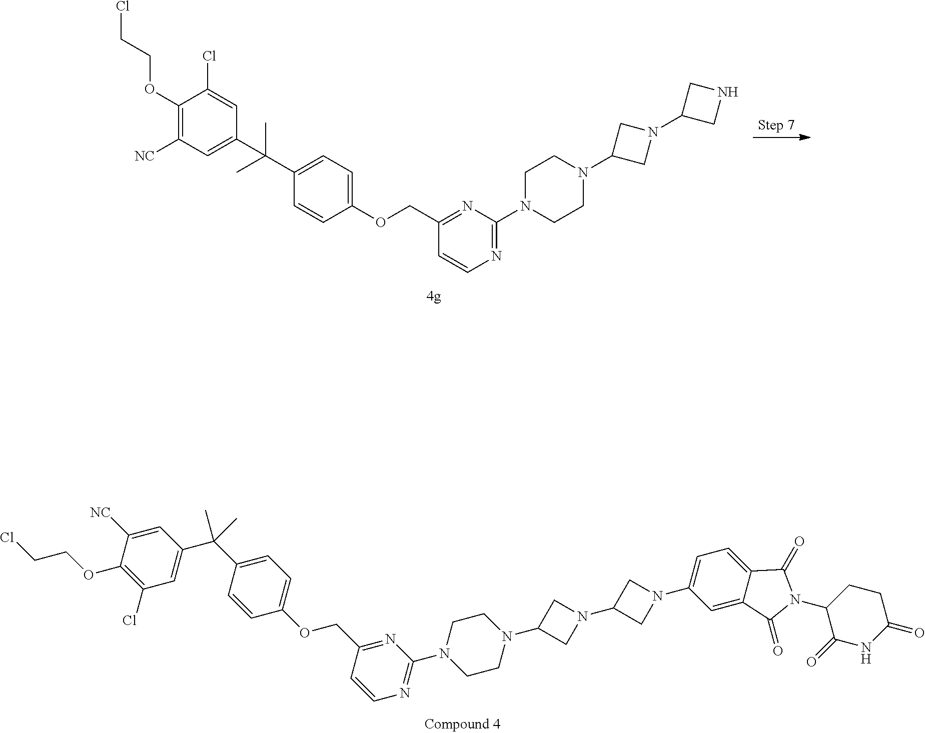 [SMILES: CC(C)(c1ccc(OCc2ccnc(N3CCN(C4CN(C5CNC5)C4)CC3)n2)cc1)c1cc(Cl)c(OCCCl)c(C#N)c1 ; CC(C)(c1ccc(OCc2ccnc(N3CCN(C4CN(C5CN(c6ccc7c(c6)C(=O)N(C6CCC(=O)NC6=O)C7=O)C5)C4)CC3)n2)cc1)c1cc(Cl)c(OCCCl)c(C#N)c1]

### Step 1: tert-butyl 4-(4-((4-(2-(3-chloro-4-(2-chloroethoxy)-5-cyanophenyl)propan-2-yl) phenoxy)methyl)pyrimidin-2-yl)piperazine-1-carboxylate (4b)

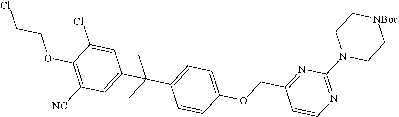 [SMILES: CC(C)(C)OC(=O)N1CCN(c2nccc(COc3ccc(C(C)(C)c4cc(Cl)c(OCCCl)c(C#N)c4)cc3)n2)CC1]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (1.68 g, 3.52 mmol) was dissolved in 60 mL of N,N′-dimethylformamide, and tert-butyl piperazine-1-carboxylate (0.98 g, 5.26 mmol) and potassium bicarbonate (0.70 mg, 7.0 mmol) were successively added. The mixture was warmed to 90° C. and stirred for 2 h. The reaction liquid was cooled to room temperature, and to the reaction liquid was slowly added 100 mL of water. The mixture was extracted with 120 mL of ethyl acetate, and the organic phase was washed with 50 mL of saturated sodium chloride solution, dried over anhydrous sodium sulfate, and concentrated under reduced pressure, to afford 4b (1.98 g, yield: 90%).

LCMS m/z=626.2 [M+1]⁺

### Step 2: 3-chloro-2-(2-chloroethoxy)-5-(2-(4-((2-(piperazin-1-yl)pyrimidin-4-yl)methoxy)phenyl)propan-2-yl)benzonitrile (4c)

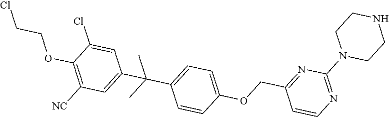 [SMILES: CC(C)(c1ccc(OCc2ccnc(N3CCNCC3)n2)cc1)c1cc(Cl)c(OCCCl)c(C#N)c1]

4b (1.98 g, 3.17 mmol) was dissolved in 5 mL of DCM, and 5 mL of trifluoroacetic acid was added.

The mixture was stirred at room temperature for 4 h. The reaction liquid was concentrated under reduced pressure, and the residue was dissolved with 50 mL of DCM, and adjusted to pH 9 with a saturated sodium bicarbonate solution. Liquid separation was performed. The aqueous phase was extracted with 100 mL of dichloromethane, and the organic phases were combined, dried over anhydrous sodium sulfate, and concentrated under reduced pressure, to afford crude 4c (1.6 g).

LCMS m/z=526.1 [M+1]⁺

### Step 3: tert-butyl 3-(4-(4-((4-(2-(3-chloro-4-(2-chloroethoxy)-5-cyanophenyl)propan-2-yl)phenoxy)methyl)pyrimidin-2-yl)piperazin-1-yl)azetidine-1-carboxylate (4d)

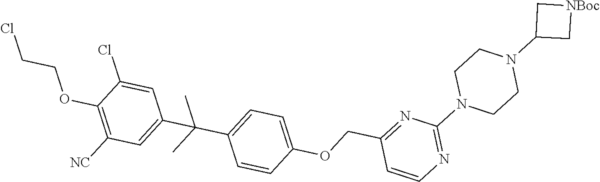 [SMILES: CC(C)(C)OC(=O)N1CC(N2CCN(c3nccc(COc4ccc(C(C)(C)c5cc(Cl)c(OCCCl)c(C#N)c5)cc4)n3)CC2)C1]

The above crude 4c (810 mg) was dissolved in 25 mL of 1,2-dichloroethane, and 1-Boc-3-azetidinone (530 mg, 3.1 mmol) was added. The mixture was stirred at room temperature for 1 h, and then sodium triacetoxyborohydride (980 mg, 4.62 mmol) was added. The resulting mixture was reacted at room temperature for 16 h. To the reaction liquid was slowly added 20 mL of saturated sodium bicarbonate solution, and the mixture was extracted with 120 mL of ethyl acetate. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 4d (950 mg, two-step total yield from compound 4b: 87%).

LCMS m/z=681.3 [M+1]⁺

### Step 4: 5-(2-(4-((2-(4-(azetidin-3-yl)piperazin-1-yl)pyrimidin-4-yl)methoxy)phenyl)propan-2-yl)-3-chloro-2-(2-chloroethoxy)benzonitrile (4e)

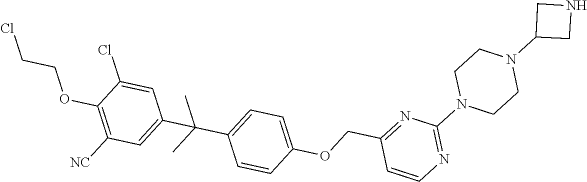 [SMILES: CC(C)(c1ccc(OCc2ccnc(N3CCN(C4CNC4)CC3)n2)cc1)c1cc(Cl)c(OCCCl)c(C#N)c1]

4d (1.1 g, 1.62 mmol) was dissolved in 15 mL of DCM, and 10 mL of trifluoroacetic acid was added. The mixture was stirred at room temperature for 4 h. The reaction liquid was concentrated under reduced pressure, dissolved with 50 mL of DCM, and adjusted to pH 9 with a saturated sodium bicarbonate solution. Liquid separation was performed. The aqueous phase was extracted with 100 mL of dichloromethane, and the organic phases were combined, dried over anhydrous sodium sulfate, and concentrated under reduced pressure, to afford crude 4e (900 mg).

LCMS m/z=581.2 [M+1]⁺

### Step 5: tert-butyl 3-(4-(4-((4-(2-(3-chloro-4-(2-chloroethoxy)-5-cyanophenyl)propan-2-yl)phenoxy)methyl)pyrimidin-2-yl)piperazin-1-yl)-[1,3′-biazetidine]-1′-carboxylate (4f)

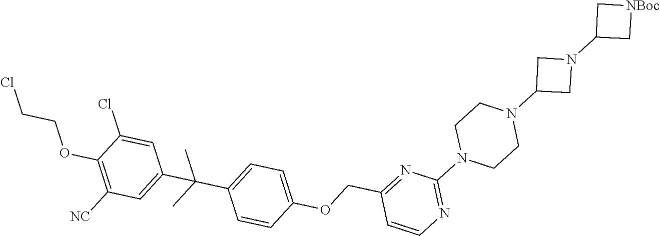 [SMILES: CC(C)(C)OC(=O)N1CC(N2CC(N3CCN(c4nccc(COc5ccc(C(C)(C)c6cc(Cl)c(OCCCl)c(C#N)c6)cc5)n4)CC3)C2)C1]

The above crude 4e (900 mg) was dissolved in 25 mL of DCE, and 1-Boc-3-azetidinone (530 mg, 3.1 mmol) was added. The mixture was stirred at room temperature for 60 min, and then sodium triacetoxyborohydride (980 mg, 4.62 mmol) was added. The resulting mixture was stirred at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 4f (700 mg, two-step total yield from compound 4d: 59%).

LCMS m/z=736.2 [M+1]⁺

### Step 6: 5-(2-(4-((2-(4-([1,3′-biazetidin]-3-yl)piperazin-1-yl)pyrimidin-4-yl)methoxy)phenyl) propan-2-yl)-3-chloro-2-(2-chloroethoxy)benzonitrile (4g)

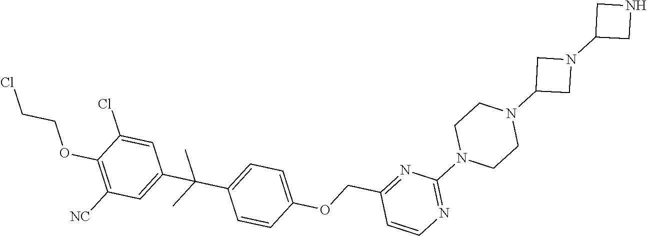 [SMILES: CC(C)(c1ccc(OCc2ccnc(N3CCN(C4CN(C5CNC5)C4)CC3)n2)cc1)c1cc(Cl)c(OCCCl)c(C#N)c1]

4f (700 mg, 0.95 mmol) was dissolved in 15 mL of DCM, and 6 mL of trifluoroacetic acid was added. The mixture was stirred at room temperature for 4 h. The reaction liquid was concentrated under reduced pressure, dissolved with 50 mL of DCM, and adjusted to pH 9 with a saturated sodium bicarbonate solution. Liquid separation was performed. The aqueous phase was extracted with 100 mL of dichloromethane, and the organic phases were combined, dried over anhydrous sodium sulfate, and concentrated under reduced pressure, to afford crude 4g (460 mg).

LCMS m/z=636.3 [M+1]⁺

### Step 7: 3-chloro-2-(2-chloroethoxy)-5-(2-(4-((2-(4-(1′-(2-(2,6-dioxopiperidin-3-yl)-1,3-dioxoisoindolin-5-yl)-[1,3′-biazetidin]-3-yl)piperazin-1-yl)pyrimidin-4-yl)methoxy)phenyl) propan-2-yl)benzonitrile (Compound 4)

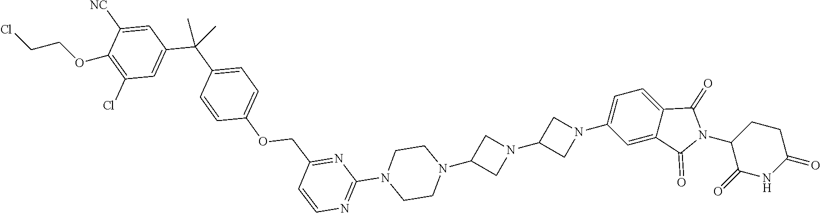 [SMILES: CC(C)(c1ccc(OCc2ccnc(N3CCN(C4CN(C5CN(c6ccc7c(c6)C(=O)N(C6CCC(=O)NC6=O)C7=O)C5)C4)CC3)n2)cc1)c1cc(Cl)c(OCCCl)c(C#N)c1]

The above crude 4g (150 mg) was dissolved in 10 mL of DMSO, and 0.2 mL of DIPEA and 2-(2,6-dioxopiperidin-3-yl)-5-fluoroisoindoline-1,3-dione (see WO 2017197056 for the synthetic method) (66 mg, 0.24 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure. The crude product was then separated and purified by silica gel column chromatography (dichloromethane/methanol (v/v)=15:1), to afford compound 4 (45 mg, two-step total yield from compound 4f: 16%).

¹H NMR (400 MHz, CDCl₃) δ 8.32 (d, 1H), 8.04 (s, 1H), 7.65 (d, 1H), 7.44 (d, 1H), 7.31 (d, 1H), 7.12-7.06 (m, 2H), 6.91-6.85 (m, 2H), 6.79 (d, 1H), 6.75 (d, 1H), 6.54 (dd, 1H), 4.98-4.88 (m, 3H), 4.41 (t, 2H), 4.11-4.03 (m, 2H), 4.00-3.53 (m, 11H), 3.38-3.02 (m, 3H), 2.93-2.69 (m, 3H), 2.60-2.26 (m, 4H), 2.18-2.08 (m, 1H), 1.63 (s, 6H).

LCMS m/z=446.7 [M/2+1]⁺

### Example 5

### 3-chloro-2-(2-chloroethoxy)-5-(2-(4-((2-(4-(1-(1-(2-(2,6-dioxopiperidin-3-yl)-1,3-dioxoisoindolin-5-yl)azetidin-3-yl)piperidin-4-yl)piperazin-1-yl)pyrimidin-4-yl)methoxy)phenyl)propan-2-yl)benzonitrile (Compound 5) trifluoroacetate

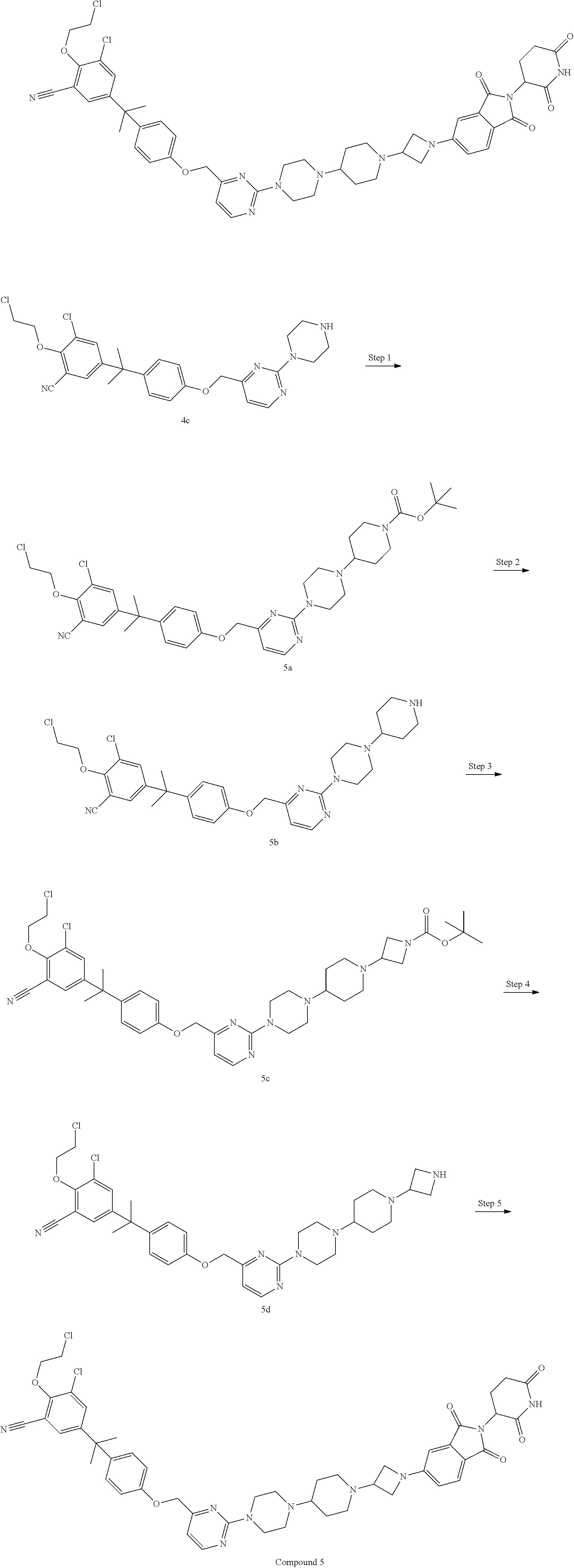

### Step 1: tert-butyl 4-(4-(4-((4-(2-(3-chloro-4-(2-chloroethoxy)-5-cyanophenyl)propan-2-yl)phenoxy)methyl)pyrimidin-2-yl)piperazin-1-yl)piperidine-1-carboxylate (5a)

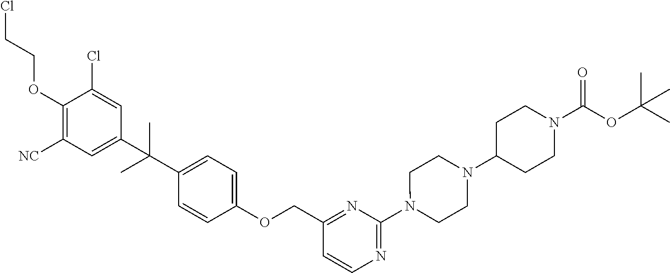 [SMILES: CC(C)(C)OC(=O)N1CCC(N2CCN(c3nccc(COc4ccc(C(C)(C)c5cc(Cl)c(OCCCl)c(C#N)c5)cc4)n3)CC2)CC1]

The above crude 4c (300 mg) was dissolved in 15 mL of 1,2-dichloroethane, and tert-butyl 4-oxopiperidine-1-carboxylate (230 mg, 1.16 mmol) was added. The mixture was stirred at room temperature for 1 h, and then sodium triacetoxyborohydride (360 mg, 1.70 mmol) was added. The resulting mixture was reacted at room temperature for 16 h. To the reaction liquid was slowly added 20 mL of saturated sodium bicarbonate solution, and the mixture was extracted with 120 mL of dichloromethane. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 5a (200 mg, two-step total yield from compound 4b: 48%).

LCMS m/z=709.3 [M+1]⁺

### Step 2: 3-chloro-2-(2-chloroethoxy)-5-(2-(4-((2-(4-(piperidin-4-yl)piperazin-1-yl)pyrimidin-4-yl)methoxy)phenyl)propan-2-yl)benzonitrile (5b)

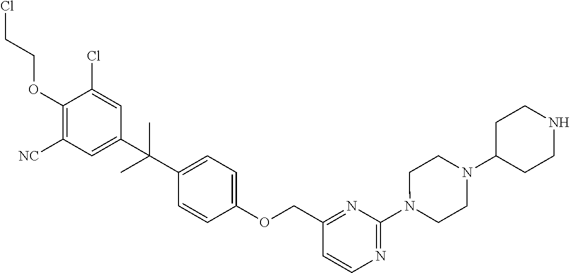 [SMILES: CC(C)(c1ccc(OCc2ccnc(N3CCN(C4CCNCC4)CC3)n2)cc1)c1cc(Cl)c(OCCCl)c(C#N)c1]

5a (0.20 g, 0.28 mmol) was dissolved in 2 mL of DCM, and 1 mL of trifluoroacetic acid was added.

The mixture was stirred at room temperature for 4 h. The reaction liquid was concentrated under reduced pressure, and the residue was dissolved with 20 mL of DCM, and adjusted to pH 8 with a saturated sodium bicarbonate solution. Liquid separation was performed. The aqueous phase was extracted with 100 mL of dichloromethane, and the organic phases were combined, dried over anhydrous sodium sulfate, and concentrated under reduced pressure, to afford crude 5b (0.15 g).

LCMS m/z=609.2 [M+1]⁺

### Step 3: tert-butyl 3-(4-(4-(4-((4-(2-(3-chloro-4-(2-chloroethoxy)-5-cyanophenyl)propan-2-yl)phenoxy)methyl)pyrimidin-2-yl)piperazin-1-yl)piperidin-1-yl)azetidine-1-carboxylate (5c)

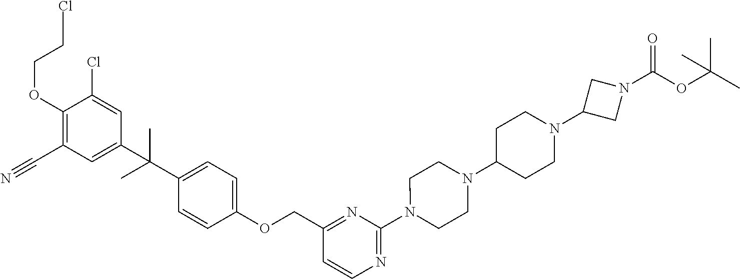 [SMILES: Cl ; CCCOc1c(Cl)cc(C(C)(C)c2ccc(OCc3ccnc(N4CCN(C5CCN(C6CN(C(=O)OC(C)(C)C)C6)CC5)CC4)n3)cc2)cc1C#N]

The above crude 3-chloro-2-(2-chloroethoxy)-5-(2-(4-((2-(4-(piperidin-4-yl)piperazin-1-yl)pyrimidin-4-yl)methoxy)phenyl)propan-2-yl)benzonitrile (5b) (150 mg) was dissolved in 15 mL of DCE, and 1-Boc-3-azetidinone (86 mg, 0.5 mmol) was added. The mixture was stirred at room temperature for 60 min, and then sodium triacetoxyborohydride (160 mg, 0.75 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 5c (180 mg, two-step total yield from compound 5a: 84%).

LCMS m/z=764.2 [M+1]⁺

### Step 4: 5-(2-(4-((2-(4-(1-(azetidin-3-yl)piperidin-4-yl)piperazin-1-yl)pyrimidin-4-yl) methoxy)phenyl)propan-2-yl)-3-chloro-2-(2-chloroethoxy)benzonitrile (5d) trifluoroacetate

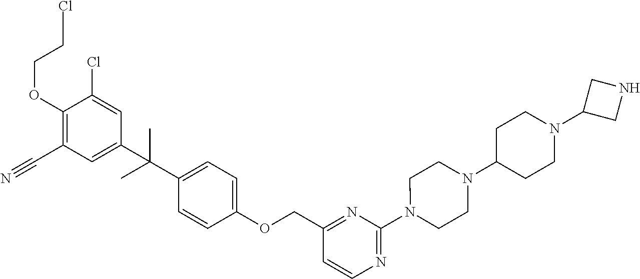 [SMILES: Cl ; CCCOc1c(Cl)cc(C(C)(C)c2ccc(OCc3ccnc(N4CCN(C5CCN(C6CNC6)CC5)CC4)n3)cc2)cc1C#N]

5c (180 mg, 0.24 mmol) was dissolved in 2 mL of DCM, and 1 mL of trifluoroacetic acid was added. The mixture was stirred at room temperature for 4 h. The reaction liquid was concentrated under reduced pressure, to afford crude 5d trifluoroacetate (185 mg).

LCMS m/z=664.3 [M+1]⁺

### Step 5: 3-chloro-2-(2-chloroethoxy)-5-(2-(4-((2-(4-(1-(1-(2-(2,6-dioxopiperidin-3-yl)-1,3-dioxoisoindolin-5-yl)azetidin-3-yl)piperidin-4-yl)piperazin-1-yl)pyrimidin-4-yl)methoxy)phenyl)propan-2-yl)benzonitrile (Compound 5) trifluoroacetate

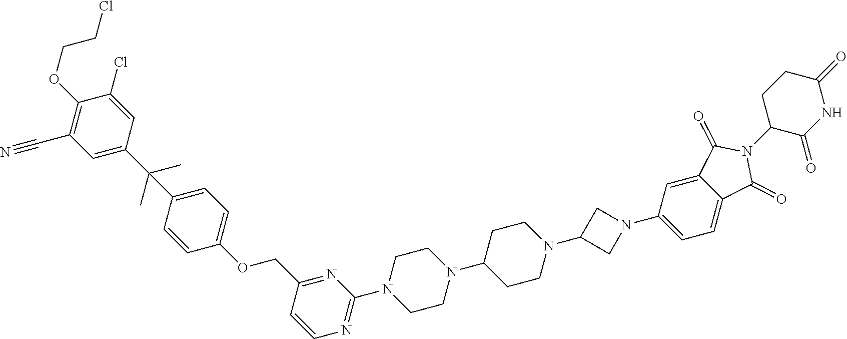 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)c(OCCCl)c(C#N)c1]

The above crude 5d trifluoroacetate (185 mg) was dissolved in 10 mL of DMSO, and solid sodium bicarbonate (100 mg, 1.19 mmol), 0.2 mL of DIPEA and 2-(2,6-dioxopiperidin-3-yl)-5-fluoroisoindoline-1,3-dione (see WO 2017197056 for the synthetic method) (66 mg, 0.24 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 5 trifluoroacetate (50 mg).

¹H NMR (400 MHz, CD₃OD) δ 8.40 (d, 1H), 7.68 (d, 1H), 7.52 (d, 1H), 7.44 (d, 1H), 7.19-7.14 (m, 2H), 6.96-6.87 (m, 4H), 6.74 (dd, 1H), 5.06 (dd, 1H), 5.02 (s, 2H), 4.47-3.93 (m, 10H), 3.93-3.84 (m, 3H), 3.56-3.34 (m, 7H), 2.92-2.80 (m, 1H), 2.78-2.67 (m, 2H), 2.67-2.56 (m, 2H), 2.44-2.34 (m, 2H), 2.15-2.05 (m, 1H), 2.04-1.91 (m, 2H), 1.65 (s, 6H).

LCMS m/z=460.8 [M/2+1]⁺.

### Example 6

### 3-chloro-2-(2-chloroethoxy)-5-(2-(4-((2-(4-(1-(1-((1-(2,6-dioxopiperidin-3-yl)-3-methyl-2-oxo-2,3-dihydro-1H-benzol[d]imidazol-4-yl)methyl)azetidin-3-yl)piperidin-4-yl)piperazin-1-yl)pyrimidin-4-yl)methoxy)phenyl)propan-2-yl)benzonitrile (Compound 6) trifluoroacetate

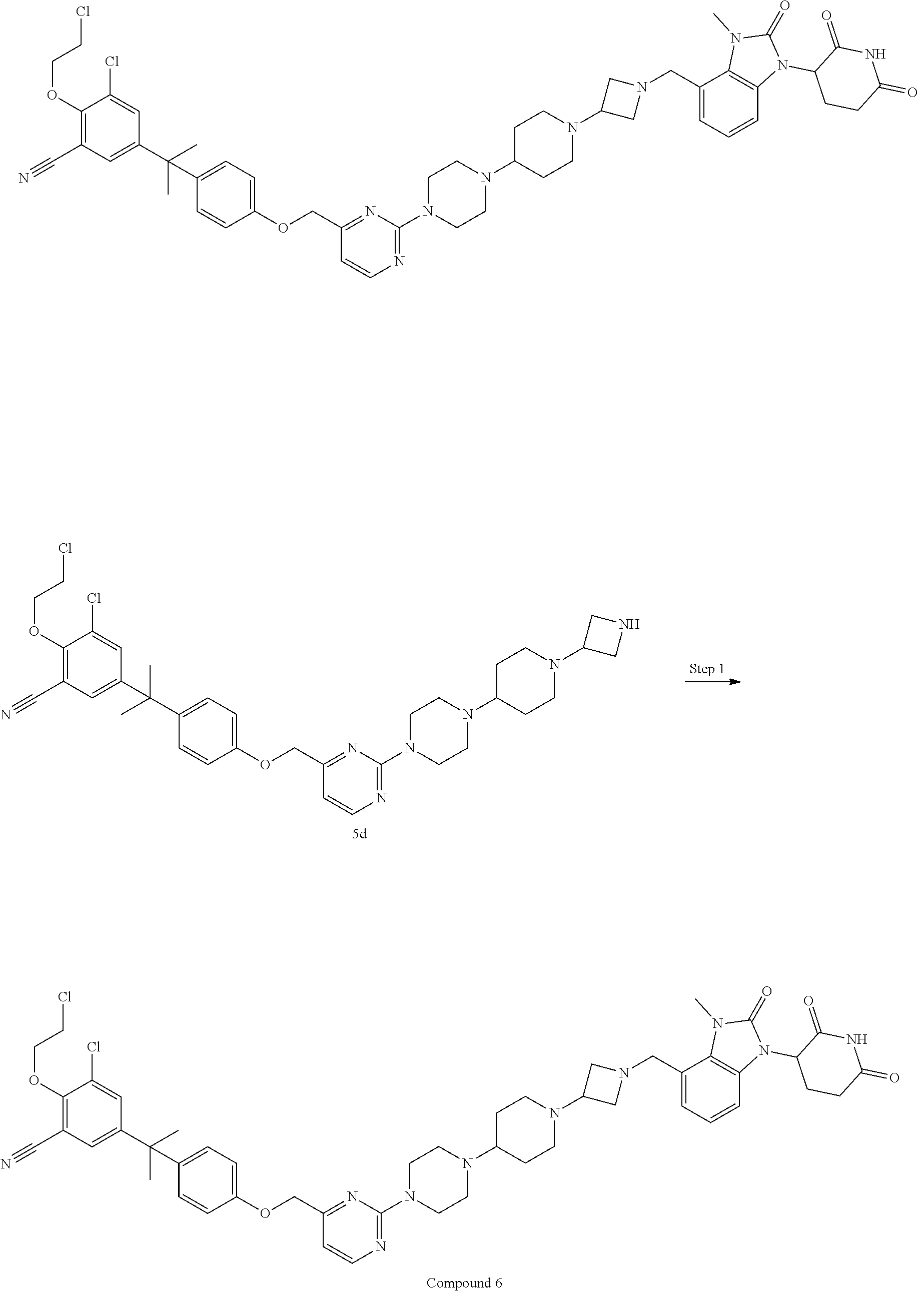 [SMILES: Cn1c(=O)n(C2CCC(=O)NC2=O)c2cccc(CN3CC(N4CCC(N5CCN(c6nccc(COc7ccc(C(C)(C)c8cc(Cl)c(OCCCl)c(C#N)c8)cc7)n6)CC5)CC4)C3)c21 ; CC(C)(c1ccc(OCc2ccnc(N3CCN(C4CCN(C5CNC5)CC4)CC3)n2)cc1)c1cc(Cl)c(OCCCl)c(C#N)c1 ; Cn1c(=O)n(C2CCC(=O)NC2=O)c2cccc(CN3CC(N4CCC(N5CCN(c6nccc(COc7ccc(C(C)(C)c8cc(Cl)c(OCCCl)c(C#N)c8)cc7)n6)CC5)CC4)C3)c21]

5d trifluoroacetate (50 mg) was dissolved in 5 mL of DMAc, and 1-(2,6-dioxopiperidin-3-yl)-3-methyl-2-oxo-2,3-dihydro-1H-benzo[d]imidazole-4-carbaldehyde (see WO 2020113233 for the synthetic method) (43 mg, 0.15 mmol) was added. The mixture was stirred at room temperature for 60 min, and then sodium triacetoxyborohydride (340 mg, 1.6 mmol) was added. The resulting mixture was stirred at room temperature for 16 h. To the reaction system was slowly added 30 mL of saturated sodium bicarbonate solution, and the mixture was extracted with ethyl acetate (60 mL×2). The organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 6 trifluoroacetate (10 mg).

¹H NMR (400 MHz, CD₃OD) δ 8.40 (d, 1H), 7.52 (d, 1H), 7.44 (d, 1H), 7.29-7.11 (m, 5H), 6.97-6.86 (m, 3H), 5.42-5.34 (m, 1H), 5.02 (s, 2H), 4.82-4.57 (m, 6H), 4.42 (t, 2H), 4.33-4.23 (m, 2H), 4.17-4.06 (m, 2H), 3.89 (t, 2H), 3.68 (s, 3H), 3.60-3.30 (m, 5H), 3.30-3.23 (m, 1H), 3.13-3.01 (m, 2H), 2.99-2.73 (m, 3H), 2.29-2.12 (m, 3H), 2.12-2.01 (m, 2H), 1.90-1.74 (m, 2H), 1.65 (s, 6H).

LCMS m/z=468.3 [M/2+1]⁺

### Example 7

### 3-chloro-2-(2-chloroethoxy)-5-(2-(4-((2-(4-(1-((1-(2-(2,6-dioxopiperidin-3-yl)-1,3-dioxoisoindolin-5-yl)piperidin-4-yl)methyl)piperidin-4-yl)piperazin-1-yl)pyrimidin-4-yl)methoxy)phenyl)propan-2-yl)benzonitrile (Compound 7)

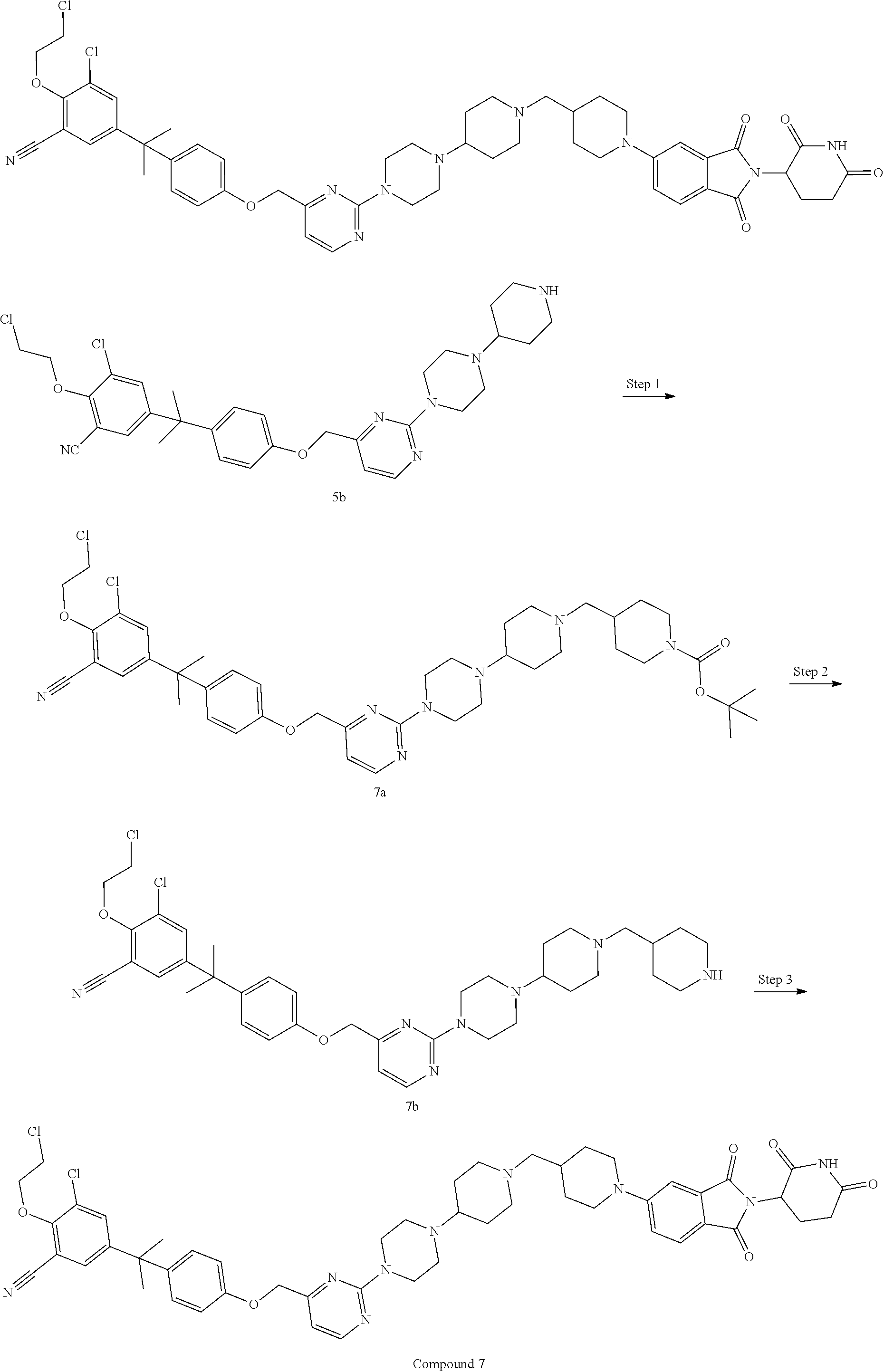

### Step 1: tert-butyl 4-((4-(4-(4-((4-(2-(3-chloro-4-(2-chloroethoxy)-5-cyanophenyl)propan-2-yl)phenoxy)methyl)pyrimidin-2-yl)piperazin-1-yl)piperidin-1-yl)methyl)piperidine-1-carboxylate (7a)

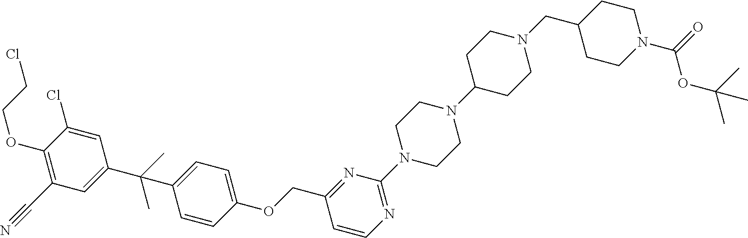 [SMILES: CC(C)(C)OC(=O)N1CCC(CN2CCC(N3CCN(c4nccc(COc5ccc(C(C)(C)c6cc(Cl)c(OCCCl)c(C#N)c6)cc5)n4)CC3)CC2)CC1]

The above crude 5b (150 mg) was dissolved in 15 mL of DCE, and tert-butyl 4-formylpiperidine-1-carboxylate (106.5 mg, 0.5 mmol) was added. The mixture was stirred at room temperature for 60 min, and then sodium triacetoxyborohydride (160 mg, 0.75 mmol) was added. The resulting mixture was reacted at room temperature for 16 h. To the reaction system was slowly added 30 mL of saturated sodium bicarbonate solution, and the mixture was extracted with 120 mL of dichloromethane. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 7a (180 mg, two-step total yield from compound 5a: 80%).

LCMS m/z=806.3 [M+1]⁺

### Step 2: 3-chloro-2-(2-chloroethoxy)-5-(2-(4-((2-(4-(1-(piperidin-4-ylmethyl)piperidin-4-yl)piperazin-1-yl)pyrimidin-4-yl)methoxy)phenyl)propan-2-yl)benzonitrile (7b) trifluoroacetate

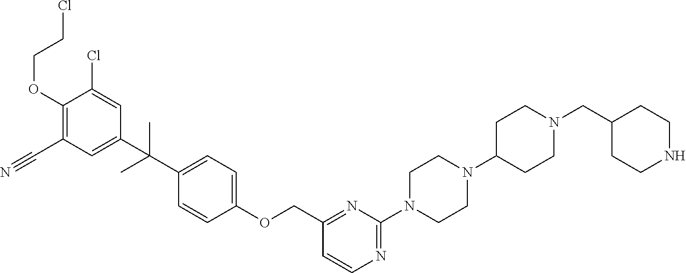 [SMILES: CC(C)(c1ccc(OCc2ccnc(N3CCN(C4CCN(CC5CCNCC5)CC4)CC3)n2)cc1)c1cc(Cl)c(OCCCl)c(C#N)c1]

7a (180 mg, 0.22 mmol) was dissolved in 2 mL of DCM, and 1 mL of trifluoroacetic acid was added. The mixture was stirred at room temperature for 4 h. The reaction liquid was concentrated under reduced pressure, to afford crude 7b trifluoroacetate (185 mg).

### Step 3: 3-chloro-2-(2-chloroethoxy)-5-(2-(4-((2-(4-(1-((1-(2-(2,6-dioxopiperidin-3-yl)-1,3-dioxoisoindolin-5-yl)piperidin-4-yl)methyl)piperidin-4-yl)piperazin-1-yl)pyrimidin-4-yl)methoxy)phenyl)propan-2-yl)benzonitrile (Compound 7)

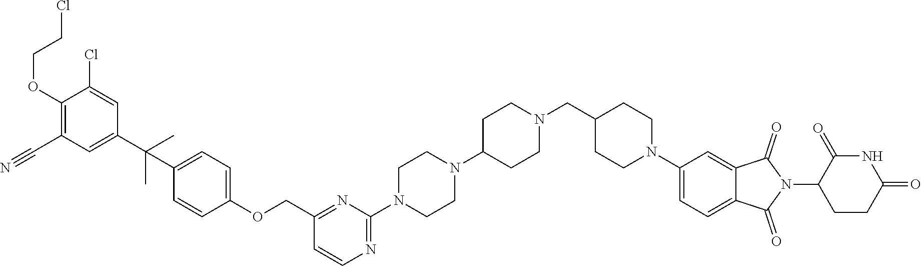 [SMILES: CC(C)(c1ccc(OCc2ccnc(N3CCN(C4CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

The above crude 7b trifluoroacetate (90 mg) was dissolved in 5 mL of DMSO, and solid sodium bicarbonate (100 mg, 1.19 mmol), 0.2 mL of DIPEA and 2-(2,6-dioxopiperidin-3-yl)-5-fluoroisoindoline-1,3-dione (see WO 2017197056 for the synthetic method) (33 mg, 0.12 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure. The resulting crude was separated and purified by a thick preparative plate (methanol/dichloromethane (v/v)=1:15), to afford compound 7 (50 mg, two-step total yield from compound 7a: 49%).

¹H NMR (400 MHz, CDCl₃) δ 8.31 (d, 1H), 8.17 (br.s, 1H), 7.66 (d, 1H), 7.44 (d, 1H), 7.32 (d, 1H), 7.27 (d, 1H), 7.12-7.00 (m, 3H), 6.92-6.84 (m, 2H), 6.71 (d, 1H), 5.00-4.88 (m, 3H), 4.42 (t, 2H), 4.00-3.75 (m, 8H), 3.05-2.92 (m, 4H), 2.92-2.60 (m, 7H), 2.45-2.28 (m, 1H), 2.28-2.15 (m, 2H), 2.15-2.07 (m, 1H), 2.06-1.94 (m, 2H), 1.94-1.85 (m, 2H), 1.73-1.61 (m, 9H), 1.35-1.17 (m, 4H).

LCMS m/z=481.8 [M/2+1]⁺

### Example 8

### 3-chloro-2-(2-chloroethoxy)-5-(2-(4-((2-(4-(1-((1-(2-(2,6-dioxopiperidin-3-yl)-1,3-dioxoisoindolin-4-yl)piperidin-4-yl)methyl)piperidin-4-yl)piperazin-1-yl)pyrimidin-4-yl)methoxy)phenyl)propan-2-yl)benzonitrile (Compound 8)

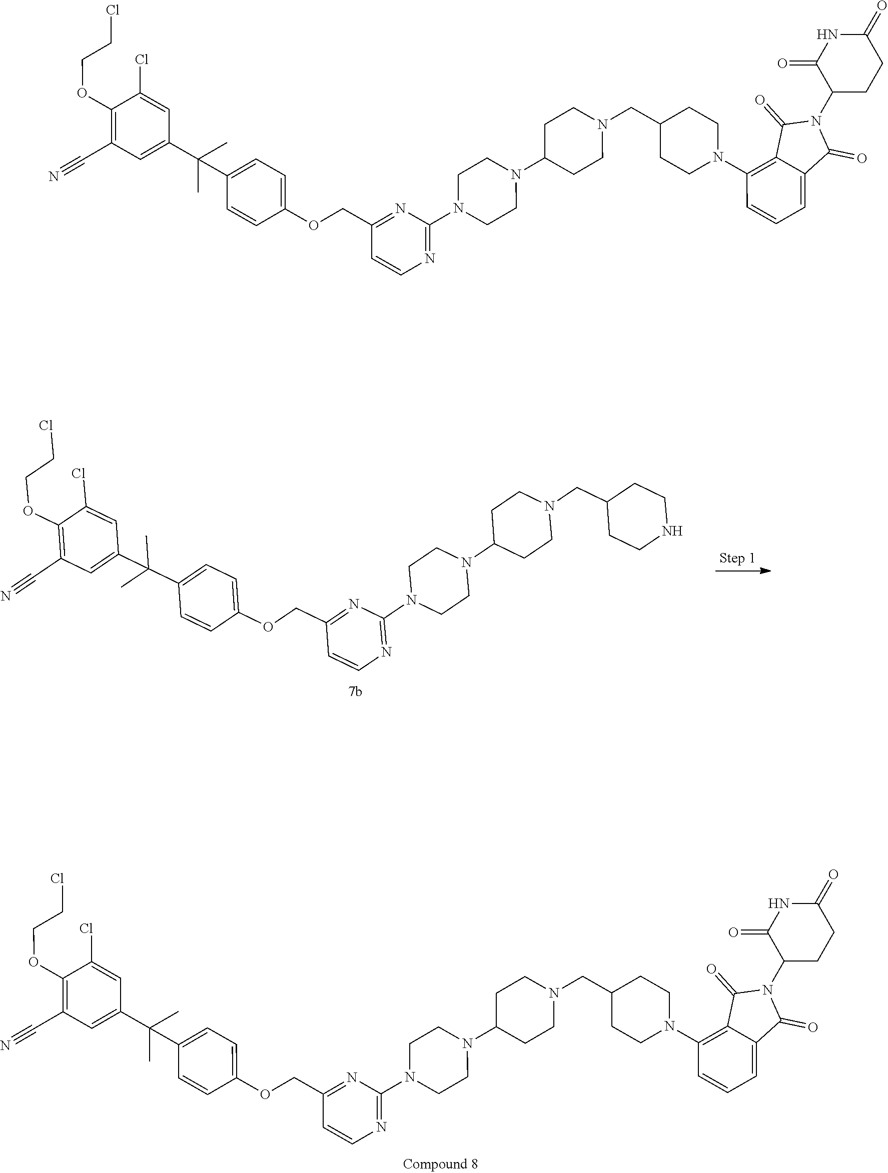 [SMILES: CC(C)(c1ccc(OCc2ccnc(N3CCN(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1 ; CC(C)(c1ccc(OCc2ccnc(N3CCN(C4CCN(CC5CCNCC5)CC4)CC3)n2)cc1)c1cc(Cl)c(OCCCl)c(C#N)c1 ; CC(C)(c1ccc(OCc2ccnc(N3CCN(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

The above crude 7b trifluoroacetate (90 mg) was dissolved in 5 mL of DMSO, and solid sodium bicarbonate (100 mg, 1.19 mmol), 0.2 mL of DIPEA and 2-(2,6-dioxopiperidin-3-yl)-4-fluoroisoindoline-1,3-dione (see WO 2017197056 for the synthetic method) (33 mg, 0.12 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure. The resulting crude was separated and purified by a thick preparative plate (methanol/dichloromethane (v/v)=1:15), to afford compound 8 (45 mg, two-step total yield from compound 7a: 44%).

¹H NMR (400 MHz, CDCl₃) δ 8.31 (d, 1H), 7.98 (br.s, 1H), 7.59-7.53 (m, 1H), 7.44 (d, 1H), 7.36 (d, 1H), 7.32 (d, 1H), 7.17 (d, 1H), 7.14-7.04 (m, 2H), 6.93-6.84 (m, 2H), 6.71 (d, 1H), 5.01-4.91 (m, 3H), 4.41 (t, 2H), 3.96-3.68 (m, 8H), 3.06-2.85 (m, 4H), 2.85-2.56 (m, 7H), 2.43-2.18 (m, 3H), 2.17-2.06 (m, 1H), 2.04-1.78 (m, 5H), 1.78-1.54 (m, 10H), 1.54-1.42 (m, 2H).

LCMS m/z=481.8 [M/2+1]⁺

### Example 9

### 3-chloro-5-(2-(4-((2-(4-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)piperazin-1-yl)pyrimidin-4-yl)methoxy)phenyl)propan-2-yl)benzonitrile (Compound 9) trifluoroacetate

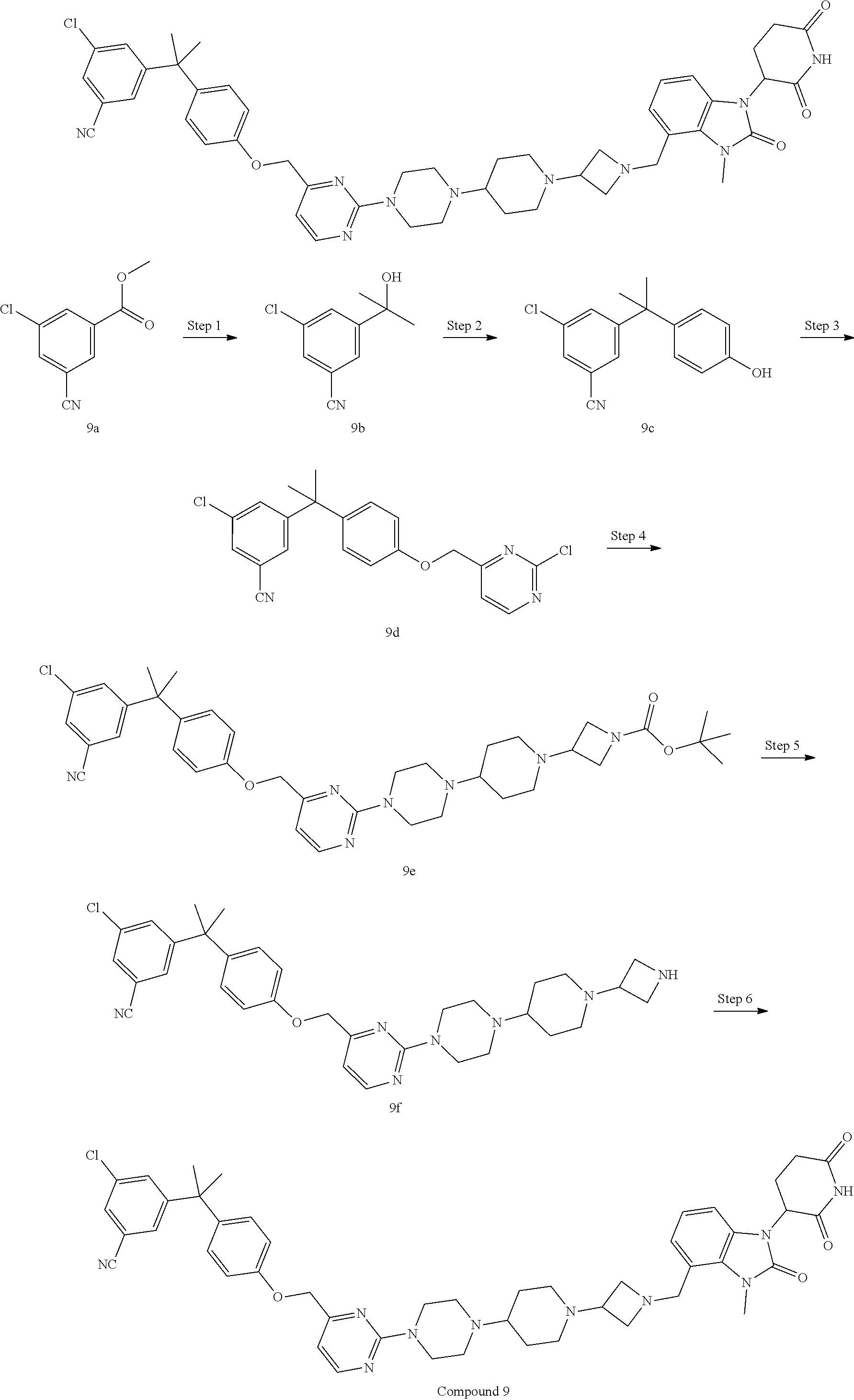

Step 1: 3-chloro-5-(2-hydroxypropan-2-yl)benzonitrile (9b)

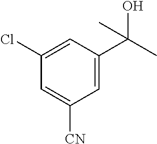

4 mL of 3 mol/L methylmagnesium bromide solution was added to 3.5 mL of tetrahydrofuran, and the mixture was cooled to 0° C. Then a solution of methyl 3-chloro-5-cyanobenzoate (9a) (0.85 g, 4.34 mmol) in tetrahydrofuran (7 mL) was added dropwise, and the mixture was stirred at 0° C. for further 2 h. At 0° C., to the reaction liquid was added dropwise 30 mL of saturated ammonium chloride solution. After the addition, the mixture was warmed to room temperature, extracted with 50 mL of ethyl acetate, washed with 50 mL of saturated sodium chloride solution, dried over anhydrous sodium sulfate, and concentrated under reduced pressure, to afford crude 3-chloro-5-(2-hydroxypropan-2-yl)benzonitrile (9b) (0.80 g).

### Step 2: 3-chloro-5-(2-(4-hydroxyphenyl)propan-2-yl)benzonitrile (9c)

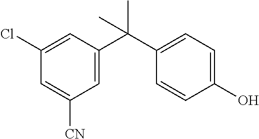 [SMILES: CC(C)(c1ccc(O)cc1)c1cc(Cl)cc(C#N)c1]

The above crude 3-chloro-5-(2-hydroxypropan-2-yl)benzonitrile (9b) (0.80 g) was dissolved in 15 mL of carbon tetrachloride, and phenol (0.42 g, 4.5 mmol) was added. The mixture was cooled to 0° C., and boron trifluoride diethyl etherate (1.16 g, 8.17 mmol) was added. The resulting mixture was then warmed to room temperature and stirred for 3 h. To the reaction liquid was slowly added 20 mL of water, and the mixture was extracted with 30 mL of dichloromethane. The organic phase was washed with 50 mL of saturated sodium chloride solution, dried over anhydrous sodium sulfate, and concentrated under reduced pressure. The crude product was separated and purified by silica gel column chromatography (ethyl acetate/petroleum ether (v/v)=0:1-1:5), to afford 3-chloro-5-(2-(4-hydroxyphenyl) propan-2-yl)benzonitrile (9c) (0.50 g, two-step total yield from compound 9a: 42%).

### Step 3: 3-chloro-5-(2-(4-((2-chloropyrimidin-4-yl)methoxy)phenyl)propan-2-yl)benzonitrile (9d)

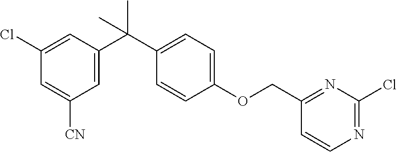 [SMILES: CC(C)(c1ccc(OCc2ccnc(Cl)n2)cc1)c1cc(Cl)cc(C#N)c1]

9c (0.50 g, 1.84 mmol) was dissolved in 15 mL of acetonitrile, and 2-chloro-4-(chloromethyl)pyrimidine (0.30 g, 1.84 mmol) and potassium carbonate (0.51 g, 3.69 mmol) were successively added. The mixture was warmed to 70° C. and stirred for 20 h. The reaction liquid was cooled to room temperature and filtered. The filtrate was concentrated under reduced pressure. The crude product was separated and purified by silica gel column chromatography (ethyl acetate/petroleum ether (v/v)=0: 1-1:5), to afford 9d (0.46 g, yield: 63%).

LCMS m/z=398.2 [M+1]⁺

### Step 4: tert-butyl 3-(4-(4-(4-((4-(2-(3-chloro-5-cyanophenyl)propan-2-yl)phenoxy)methyl)pyrimidin-2-yl)piperazin-1-yl)piperidin-1-yl)azetidine-1-carboxylate (9e)

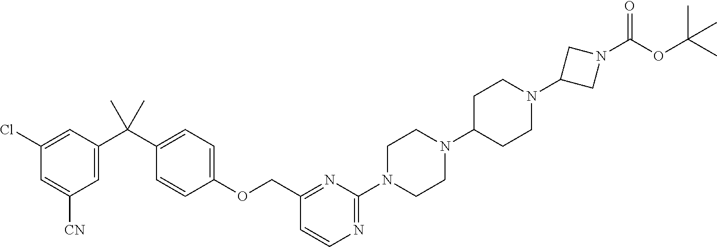 [SMILES: CC(C)(C)OC(=O)N1CC(N2CCC(N3CCN(c4nccc(COc5ccc(C(C)(C)c6cc(Cl)cc(C#N)c6)cc5)n4)CC3)CC2)C1]

9d (250 mg, 0.63 mmol) was dissolved in 10 mL of DMF, and the above crude tert-butyl 3-(4-(piperazin-1-yl)piperidin-1-yl)azetidine-1-carboxylate (intermediate 1) (200 mg) and solid potassium bicarbonate (133 mg, 1.33 mmol) were successively added. The mixture was warmed to 90° C. and stirred for 4 h. The mixture was cooled to room temperature, and 30 mL of water was added. The resulting mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 9e (280 mg, yield from compound 9d: 65%).

LCMS m/z=686.3 [M+1]⁺

### Step 5: 3-(2-(4-((2-(4-(1-(azetidin-3-yl)piperidin-4-yl)piperazin-1-yl)pyrimidin-4-yl)methoxy)phenyl)propan-2-yl)-5-chlorobenzonitrile (9f)

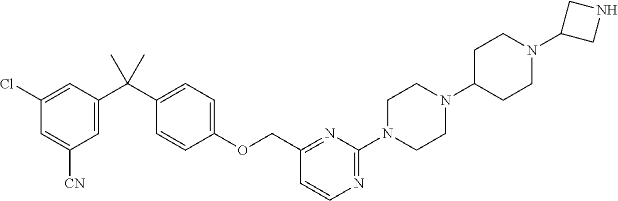 [SMILES: CC(C)(c1ccc(OCc2ccnc(N3CCN(C4CCN(C5CNC5)CC4)CC3)n2)cc1)c1cc(Cl)cc(C#N)c1]

9e (280 mg, 0.41 mmol) was dissolved in 5 mL of dichloromethane, and 2 mL of trifluoroacetic acid was added. The mixture was stirred at room temperature for 4 h. The reaction system was concentrated under reduced pressure, and 50 mL of dichloromethane was added. The mixture was adjusted to pH 9 with a saturated sodium bicarbonate solution, extracted with 50 mL of dichloromethane, dried over anhydrous sodium sulfate, and concentrated under reduced pressure, to afford crude 9f (200 mg).

LCMS m/z=586.3 [M+1]⁺

### Step 6: 3-chloro-5-(2-(4-((2-(4-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)piperazin-1-yl)pyrimidin-4-yl)methoxy)phenyl)propan-2-yl)benzonitrile (Compound 9) trifluoroacetate

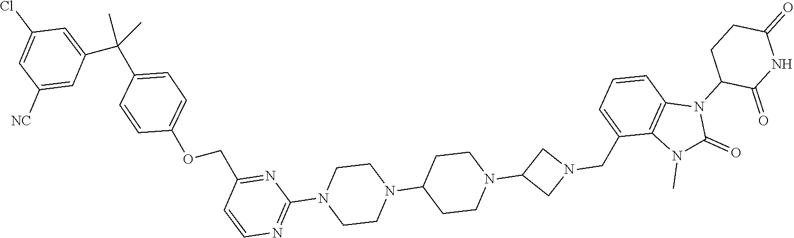 [SMILES: Cn1c(=O)n(C2CCC(=O)NC2=O)c2cccc(CN3CC(N4CCC(N5CCN(c6nccc(COc7ccc(C(C)(C)c8cc(Cl)cc(C#N)c8)cc7)n6)CC5)CC4)C3)c21]

The above crude 9f (46 mg) was dissolved in 5 mL of DMAc, and 1-(2,6-dioxopiperidin-3-yl)-3-methyl-2-oxo-2,3-dihydro-1H-benzo[d]imidazole-4-carbaldehyde (see WO 2020113233 for the synthetic method) (23 mg, 0.08 mmol) was added. The mixture was stirred at room temperature for 60 min, and then sodium triacetoxyborohydride (230 mg, 1.09 mmol) was added. The resulting mixture was stirred at room temperature for 16 h. To the reaction system was slowly added 30 mL of saturated sodium bicarbonate solution, and the mixture was extracted twice with 60 mL of ethyl acetate. The organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 9 trifluoroacetate (8 mg).

¹H NMR (400 MHz, DMSO-d₆) δ 11.11 (s, 1H), 8.46 (d, 1H), 7.86-7.82 (m, 1H), 7.68-7.62 (m, 1H), 7.56-7.50 (m, 1H), 7.32 (s, 1H), 7.26-7.12 (m, 4H), 6.99-6.91 (m, 2H), 6.85 (d, 1H), 5.40 (dd, 1H), 5.02 (s, 2H), 4.98-4.44 (m, 4H), 4.40 (s, 2H), 4.19-4.01 (m, 4H), 3.53-3.41 (m, 1H), 3.36 (s, 3H), 3.34-2.98 (m, 9H), 2.98-2.84 (m, 1H), 2.81-2.58 (m, 2H), 2.22-1.96 (m, 3H), 1.79-1.58 (m, 8H).

LCMS m/z=429.3 [M/2+1]⁺

### Example 10

### 3-chloro-5-(2-(4-((2-(4-(1-(1-(2-(2,6-dioxopiperidin-3-yl)-1,3-dioxoisoindolin-5-yl) azetidin-3-yl)piperidin-4-yl)piperazin-1-yl)pyrimidin-4-yl)methoxy)phenyl)propan-2-yl)benzonitrile (Compound 10)

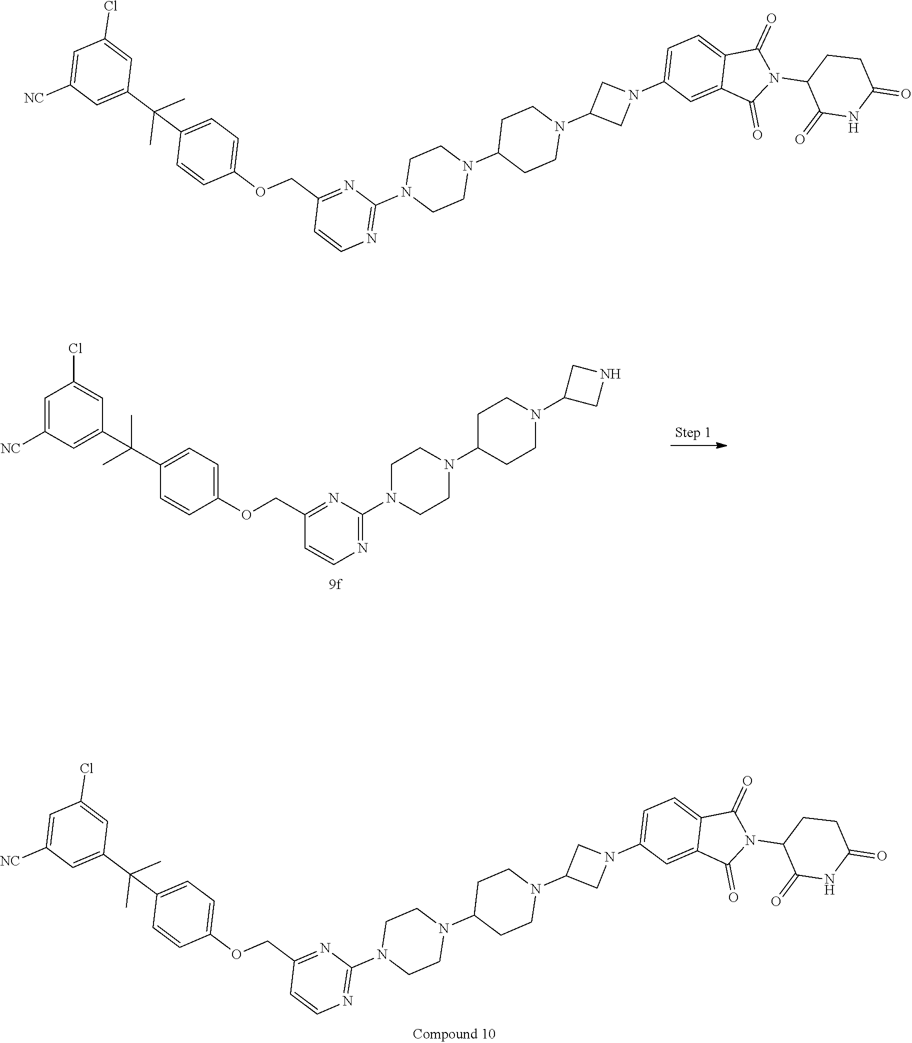 [SMILES: CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)cc(C#N)c1 ; CC(C)(c1ccc(OCc2ccnc(N3CCN(C4CCN(C5CNC5)CC4)CC3)n2)cc1)c1cc(Cl)cc(C#N)c1 ; CC(C)(c1ccc(OCc2ccnc(N3CCN(C4CCN(C5CN(c6ccc7c(c6)C(=O)N(C6CCC(=O)NC6=O)C7=O)C5)CC4)CC3)n2)cc1)c1cc(Cl)cc(C#N)c1]

The above crude 9f (50 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (66 mg, 0.24 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure. The resulting crude product was separated and purified by silica gel column chromatography (dichloromethane/methanol (v/v)=15:1), to afford compound 10 (16 mg, two-step total yield from compound 9e: 19%).

¹H NMR (400 MHz, CDCl₃) δ 8.23 (d, 1H), 8.08-7.98 (m, 1H), 7.57 (d, 1H), 7.39-7.33 (m, 2H), 7.32-7.27 (m, 1H), 7.06-6.96 (m, 2H), 6.85-6.77 (m, 2H), 6.71 (d, 1H), 6.64 (d, 1H), 6.45 (dd, 1H), 4.93-4.77 (m, 3H), 4.03 (t, 2H), 3.94-3.60 (m, 6H), 3.34-3.22 (m, 1H), 2.95-2.85 (m, 2H), 2.85-2.48 (m, 7H), 2.45-2.26 (m, 1H), 2.11-2.00 (m, 1H), 1.94-1.79 (m, 2H), 1.70-1.45 (m, 10H).

LCMS m/z=842.3 [M+1]⁺

### Example 11

### 3-chloro-2-(2-chloroethoxy)-5-(2-(4-((2-(4-((1′-(2-(2,6-dioxopiperidin-3-yl)-1,3-dioxoisoindolin-5-yl)-[1,4′-bipiperidin]-4-yl)methyl)piperazin-1-yl)pyrimidin-4-yl)methoxy)phenyl)propan-2-yl)benzonitrile (Compound 11)

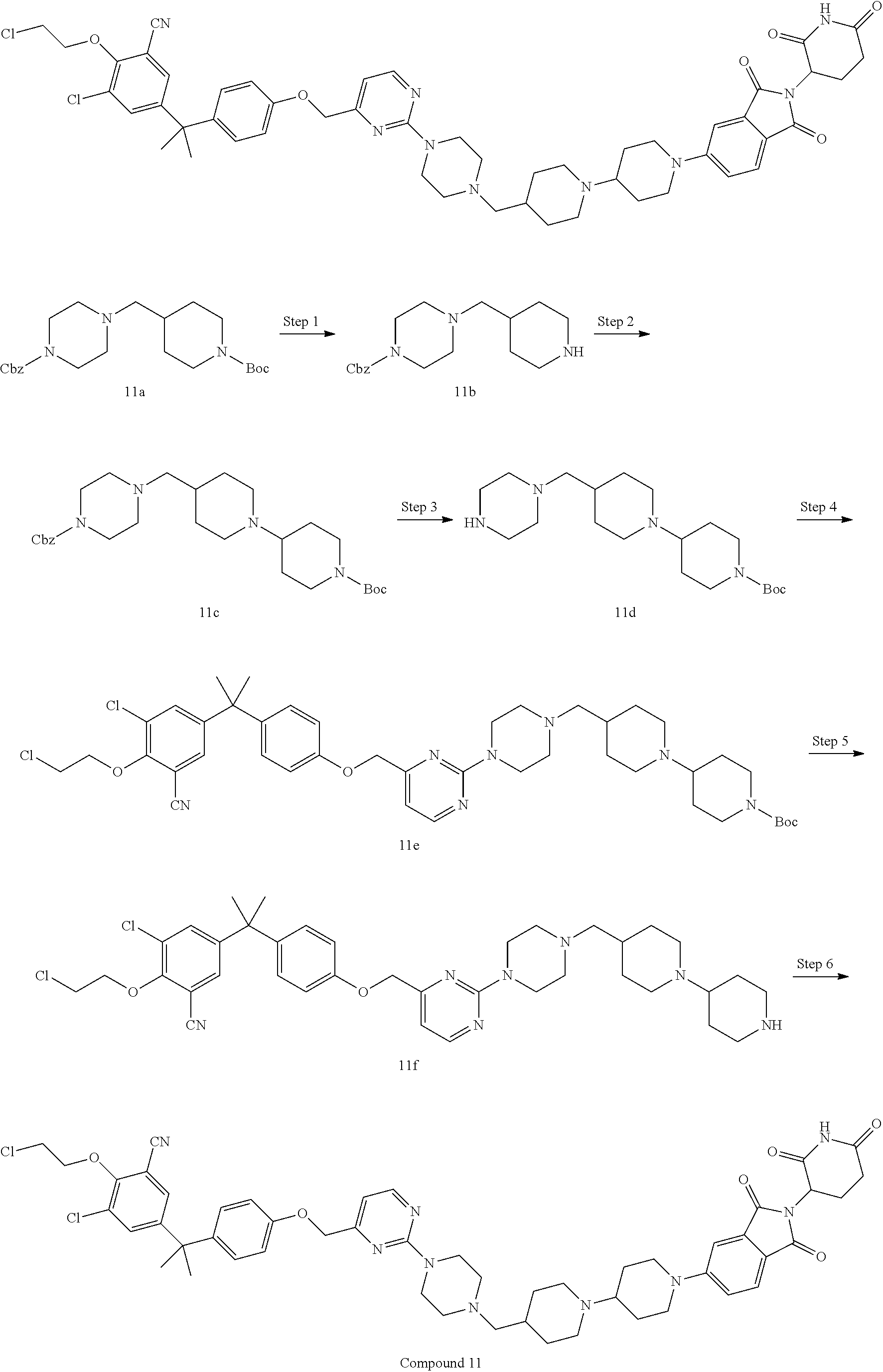

### Step 1: Benzyl 4-(piperidin-4-ylmethyl)piperazine-1-carboxylate (11b)

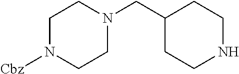 [SMILES: O=C(OCc1ccccc1)N1CCN(CC2CCNCC2)CC1]

Benzyl 4-((1-(tertbutoxycarbonyl)piperidin-4-yl)methyl)piperazine-1-carboxylate (11a) (see WO 2017095758 for the synthetic method) (5.5 g, 13.17 mmol) was dissolved in 100 mL of dichloromethane, and 20 mL of trifluoroacetic acid was added. The mixture was stirred at room temperature for 2 h. The reaction system was concentrated under reduced pressure, and to the residue was added 100 mL of aqueous sodium bicarbonate solution. The mixture was extracted with dichloromethane (80 mL×3), and the organic phase was washed with 100 mL of water, dried over anhydrous sodium sulfate, and concentrated under reduced pressure, to afford crude 11b (3.4 g).

LCMS m/z=318.2 [M+1]⁺

### Step 2: tert-butyl 4-((4-((benzyloxy)carbonyl)piperazin-1-yl)methyl)-[1,4′-bipiperidine]-1′-carboxylate (11c)

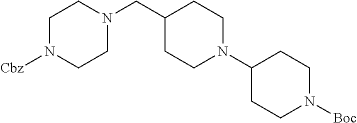 [SMILES: CC(C)(C)OC(=O)N1CCC(N2CCC(CN3CCN(C(=O)OCc4ccccc4)CC3)CC2)CC1]

The above crude 11b (1.0 g) was dissolved in 35 mL of DCE, and tert-butyl 4-oxopiperidine-1-carboxylate (1.38 g, 6.94 mmol) was added. The mixture was stirred at room temperature for 30 min, and then sodium triacetoxyborohydride (1.47 g, 6.94 mmol) was added. The resulting mixture was stirred at room temperature for 16 h. To the reaction system was slowly added 50 mL of saturated aqueous sodium bicarbonate solution, and the mixture was extracted with dichloromethane (60 mL×3). The organic phase was washed with 50 mL of water, dried over anhydrous sodium sulfate, and concentrated under reduced pressure. The crude product was separated and purified by silica gel column chromatography (dichloromethane/methanol (v/v)=15:1-10:1), to afford 11c (1.2 g, two-step total yield from compound 11a: 62%).

LCMS m/z=501.3 [M+1]⁺

### Step 3: tert-butyl 4-(piperazin-1-ylmethyl)-[1,4′-bipiperidine]-1′-carboxylate (11d)

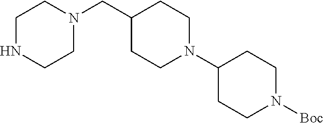 [SMILES: CC(C)(C)OC(=O)N1CCC(N2CCC(CN3CCNCC3)CC2)CC1]

11c (600 mg, 1.20 mmol) was dissolved in 25 mL of methanol, and 0.15 g of 10% palladium on carbon was added. The mixture was stirred at room temperature under hydrogen atmosphere for 16 h. The reaction liquid was filtered, and the filter cake was washed with methanol (5 mL×3). The filtrate was collected and concentrated under reduced pressure, to afford crude 11d (0.45 g).

LCMS m/z=367.3 [M+1]⁺

### Step 4: tert-butyl 4-((4-(4-((4-(2-(3-chloro-4-(2-chloroethoxy)-5-cyanophenyl)propan-2-yl)phenoxy)methyl)pyrimidin-2-yl)piperazin-1-yl)methyl)-[1,4′-bipiperidine]-1′-carboxylate (11e)

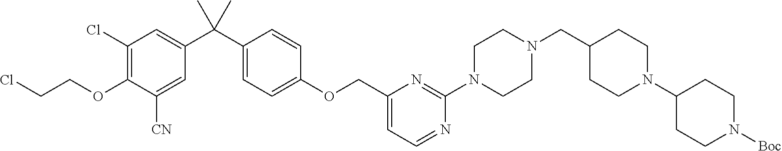 [SMILES: CC(C)(C)OC(=O)N1CCC(N2CCC(CN3CCN(c4nccc(COc5ccc(C(C)(C)c6cc(Cl)c(OCCCl)c(C#N)c6)cc5)n4)CC3)CC2)CC1]

The above crude 3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (250 mg, 0.524 mmol) was dissolved in 15 ml of DMF, and solid potassium bicarbonate (105 mg, 1.05 mmol) and the crude 11d from the previous step (225 mg) were added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 11e (300 mg, two-step total yield from compound 11c: 62%).

LCMS m/z=806.3 [M+1]⁺

### Step 5: 5-(2-(4-((2-(4-([1,4′-bipiperidin]-4-ylmethyl)piperazin-1-yl)pyrimidin-4-yl)methoxy)phenyl)propan-2-yl)-3-chloro-2-(2-chloroethoxy)benzonitrile (11f) trifluoroacetate

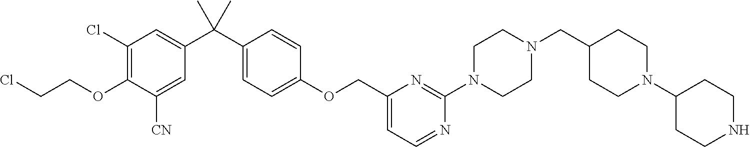 [SMILES: CC(C)(c1ccc(OCc2ccnc(N3CCN(CC4CCN(C5CCNCC5)CC4)CC3)n2)cc1)c1cc(Cl)c(OCCCl)c(C#N)c1]

11e (60 mg, 0.074 mmol) was dissolved in 5 mL of DCM, and 2 mL of trifluoroacetic acid was added. The mixture was stirred at room temperature for 2 h. The reaction liquid was concentrated under reduced pressure, to afford crude 11f trifluoroacetate (70 mg).

LCMS m/z=706.3 [M+1]⁺

### Step 6: 3-chloro-2-(2-chloroethoxy)-5-(2-(4-((2-(4-((1′-(2-(2,6-dioxopiperidin-3-yl)-1,3-dioxoisoindolin-5-yl)-[1,4′-bipiperidin]-4-yl)methyl)piperazin-1-yl)pyrimidin-4-yl)methoxy)phenyl)propan-2-yl)benzonitrile (Compound 11)

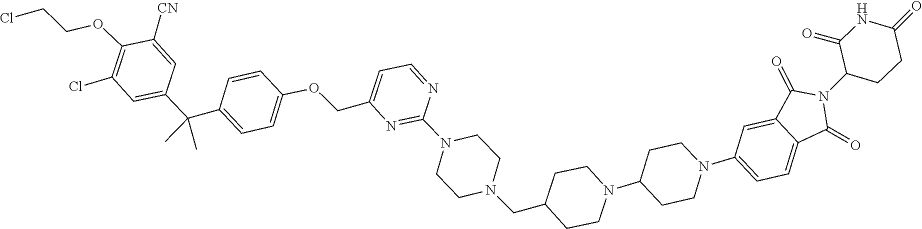 [SMILES: CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)c(OCCCl)c(C#N)c1]

The above crude 11f trifluoroacetate (70 mg) was dissolved in 4 mL of DMSO, and solid sodium bicarbonate (32 mg, 0.38 mmol) was added. The mixture was stirred at room temperature for 10 min, and then 0.5 mL of DIPEA and 2-(2,6-dioxopiperidin-3-yl)-5-fluoroisoindoline-1,3-dione (see WO 2017197056 for the synthetic method) (41 mg, 0.15 mmol) were added. The resulting mixture was warmed to 80° C. and stirred for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=15:1), to afford compound 11 (25 mg, two-step total yield from compound 11e: 35%).

¹H NMR (400 MHz, CDCl₃) δ 8.30 (d, 1H), 8.01 (br.s, 1H), 7.68 (d, 1H), 7.44 (d, 1H), 7.32 (d, 1H), 7.28 (d, 1H), 7.12-7.02 (m, 3H), 6.91-6.85 (m, 2H), 6.70 (d, 1H), 4.98-4.89 (m, 3H), 4.41 (t, 2H), 4.07-3.97 (m, 2H), 3.90-3.76 (m, 6H), 3.20-2.65 (m, 8H), 2.50-2.40 (m, 4H), 2.31-2.20 (m, 3H), 2.18-1.98 (m, 3H), 1.96-1.82 (m, 2H), 1.82-1.48 (m, 12H).

LCMS m/z=962.3 [M+1]⁺

### Example 12

### 3-chloro-2-(2-chloroethoxy)-5-(2-(4-((2-(4-((1′-(2-(2,6-dioxopiperidin-3-yl)-1,3-dioxoisoindolin-4-yl)-[1,4′-bipiperidin]-4-yl)methyl)piperazin-1-yl)pyrimidin-4-yl)methoxy)phenyl)propan-2-yl)benzonitrile (Compound 12)

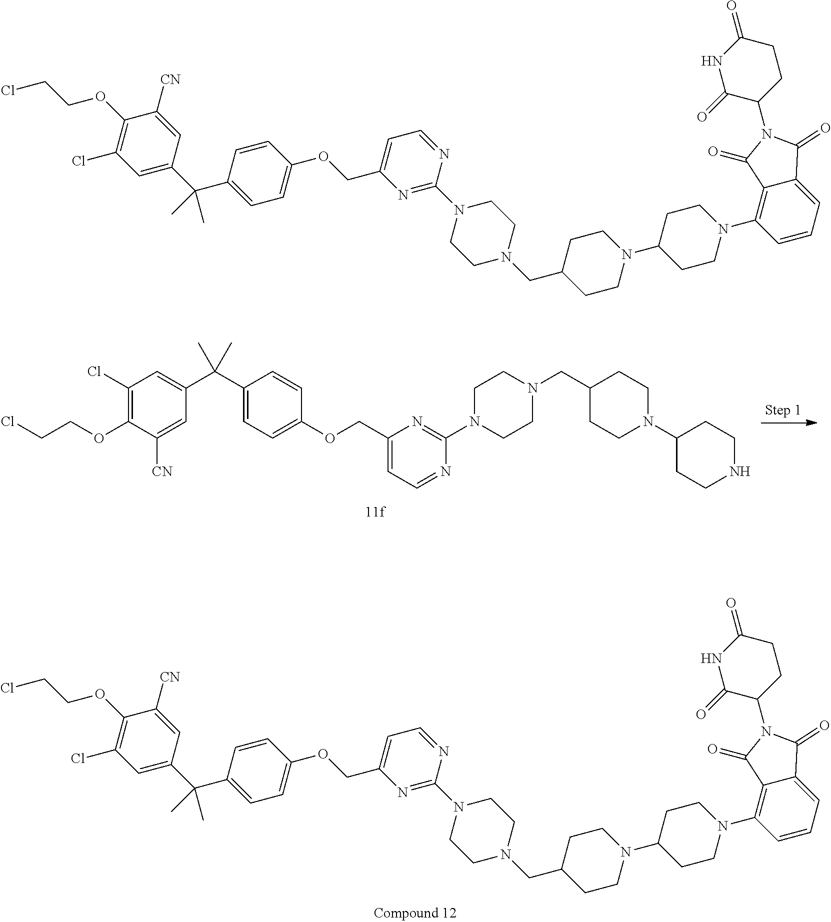 [SMILES: CC(C)(c1ccc(OCc2ccnc(N3CCN(CC4CCN(C5CCN(c6cccc7c6C(=O)N(C6CCC(=O)NC6=O)C7=O)CC5)CC4)CC3)n2)cc1)c1cc(Cl)c(OCCCl)c(C#N)c1 ; CC(C)(c1ccc(OCc2ccnc(N3CCN(CC4CCN(C5CCNCC5)CC4)CC3)n2)cc1)c1cc(Cl)c(OCCCl)c(C#N)c1 ; CC(C)(c1ccc(OCc2ccnc(N3CCN(CC4CCN(C5CCN(c6cccc7c6C(=O)N(C6CCC(=O)NC6=O)C7=O)CC5)CC4)CC3)n2)cc1)c1cc(Cl)c(OCCCl)c(C#N)c1]

The above crude 11f trifluoroacetate (60 mg) was dissolved in 4 mL of DMSO, and solid sodium bicarbonate (26 mg, 0.31 mmol) was added. The mixture was stirred at room temperature for 10 min, and then 0.5 mL of DIPEA and 2-(2,6-dioxopiperidin-3-yl)-4-fluoroisoindoline-1,3-dione (see WO 2017197056 for the synthetic method) (34 mg, 0.12 mmol) were added. The resulting mixture was warmed to 80° C. and stirred for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=15:1), to afford compound 12 (20 mg, two-step total yield from compound 11e: 33%).

¹H NMR (400 MHz, CDCl₃) δ 8.30 (d, 1H), 8.00 (br.s, 1H), 7.62-7.54 (m, 1H), 7.46-7.36 (m, 2H), 7.32 (d, 1H), 7.16 (d, 1H), 7.12-7.06 (m, 2H), 6.92-6.85 (m, 2H), 6.70 (d, 1H), 5.01-4.91 (m, 3H), 4.42 (t, 2H), 3.96-3.75 (m, 8H), 3.25-2.65 (m, 8H), 2.54-2.38 (m, 5H), 2.34-2.06 (m, 5H), 2.04-1.75 (m, 5H), 1.75-1.48 (m, 9H).

LCMS m/z=962.3 [M+1]⁺

### Example 13

### 3-chloro-2-(2-chloroethoxy)-5-(2-(4-((2-(4-((1-((1-(2-(2,6-dioxopiperidin-3-yl)-1,3-dioxoisoindolin-5-yl)piperidin-4-yl)methyl)piperidin-4-yl)methyl)piperazin-1-yl)pyrimidin-4-yl)methoxy)phenyl)propan-2-yl)benzonitrile (Compound 13)

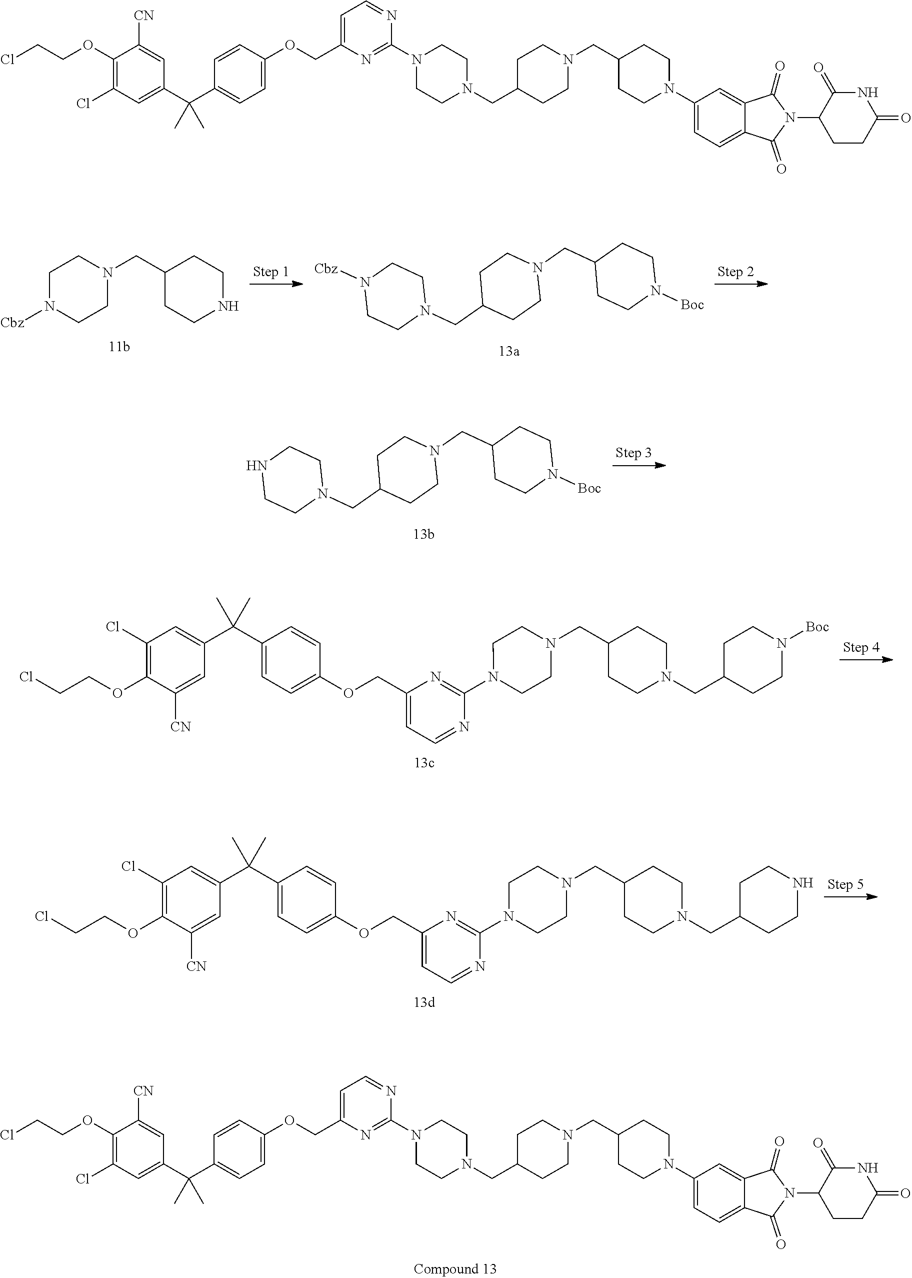

### Step 1: benzyl 4-((1-((1-(tert-butoxycarbonyl)piperidin-4-yl)methyl)piperidin-4-yl)methyl) piperazine-1-carboxylate (13a)

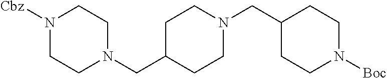 [SMILES: CC(C)(C)OC(=O)N1CCC(CN2CCC(CN3CCN(C(=O)OCc4ccccc4)CC3)CC2)CC1]

The above crude benzyl 4-(piperidin-4-ylmethyl)piperazine-1-carboxylate (11b) (0.75 g) was dissolved in 35 mL of DCE, and tert-butyl 4-formylpiperidine-1-carboxylate (1.05 g, 4.92 mmol) was added. The mixture was stirred at room temperature for 30 min, and then sodium triacetoxyborohydride (1.02 g, 4.81 mmol) was added. The resulting mixture was stirred at room temperature for 16 h. To the reaction system was slowly added 50 mL of saturated aqueous sodium bicarbonate solution, and the mixture was extracted with dichloromethane (60 mL×3). The organic phase was washed with 50 mL of water, dried over anhydrous sodium sulfate, and concentrated under reduced pressure. The crude product was separated and purified by silica gel column chromatography (dichloromethane/methanol (v/v)=15:1-10:1), to afford 13a (1.0 g, two-step total yield from compound 11a: 67%).

LCMS m/z=515.3 [M+1]⁺

### Step 2: tert-butyl 4-((4-(piperazin-1-ylmethyl)piperidin-1-yl)methyl)piperidine-1-carboxylate (13b)

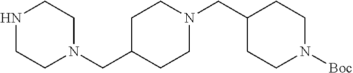 [SMILES: CC(C)(C)OC(=O)N1CCC(CN2CCC(CN3CCNCC3)CC2)CC1]

13a (600 mg, 1.17 mmol) was dissolved in 25 mL of methanol, and 0.15 g of 10% palladium on carbon was added. The mixture was stirred at room temperature under hydrogen atmosphere for 16 h. The reaction liquid was filtered, and the filter cake was washed with methanol (5 mL×3). The filtrate was collected and concentrated under reduced pressure, to afford crude 13b (0.43 g).

LCMS m/z=381.3 [M+1]⁺

### Step 3: tert-butyl 4-((4-((4-(4-((4-(2-(3-chloro-4-(2-chloroethoxy)-5-cyanophenyl)propan-2-yl)phenoxy)methyl)pyrimidin-2-yl)piperazin-1-yl)methyl)piperidin-1-yl)methyl) piperidine-1-carboxylate (13c)

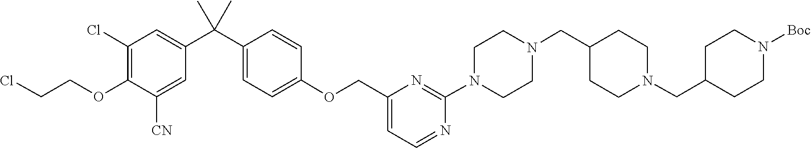 [SMILES: CC(C)(C)OC(=O)N1CCC(CN2CCC(CN3CCN(c4nccc(COc5ccc(C(C)(C)c6cc(Cl)c(OCCCl)c(C#N)c6)cc5)n4)CC3)CC2)CC1]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (250 mg, 0.524 mmol) was dissolved in 15 mL of DMF, and solid potassium bicarbonate (105 mg, 1.05 mmol) and the crude 13b from the previous step (230 mg) were added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 13c (290 mg, two-step total yield from compound 13a: 57%).

LCMS m/z=820.3 [M+1]⁺

### Step 4: 3-chloro-2-(2-chloroethoxy)-5-(2-(4-((2-(4-((1-(piperidin-4-ylmethyl)piperidin-4-yl)methyl)piperazin-1-yl)pyrimidin-4-yl)methoxy)phenyl)propan-2-yl)benzonitrile (13d) trifluoroacetate

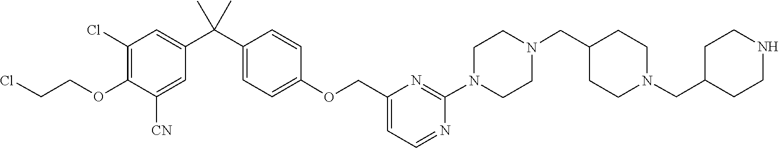 [SMILES: CC(C)(c1ccc(OCc2ccnc(N3CCN(CC4CCN(CC5CCNCC5)CC4)CC3)n2)cc1)c1cc(Cl)c(OCCCl)c(C#N)c1]

13c (50 mg, 0.061 mmol) was dissolved in 5 mL of DCM, and 2 mL of trifluoroacetic acid was added. The mixture was stirred at room temperature for 2 h. The reaction liquid was concentrated under reduced pressure, to afford crude 13d trifluoroacetate (60 mg).

LCMS m/z=720.3 [M+1]⁺

### Step 5: 3-chloro-2-(2-chloroethoxy)-5-(2-(4-((2-(4-((1-((1-(2-(2,6-dioxopiperidin-3-yl)-1,3-dioxoisoindolin-5-yl)piperidin-4-yl)methyl)piperidin-4-yl)methyl)piperazin-1-yl)pyrimidin-4-yl)methoxy)phenyl)propan-2-yl)benzonitrile (Compound 13)

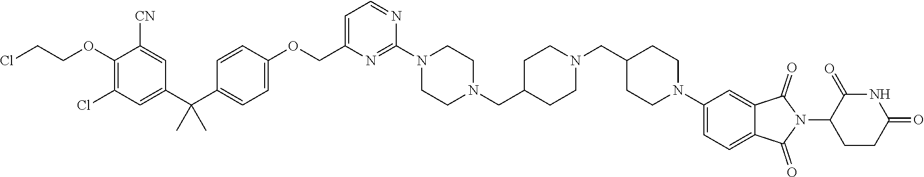 [SMILES: CC(C)(c1ccc(OCc2ccnc(N3CCN(CC4CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

The above crude 13d trifluoroacetate (60 mg) was dissolved in 4 mL of DMSO, and solid sodium bicarbonate (26 mg, 0.31 mmol) was added. The mixture was stirred at room temperature for 10 min, and then 0.5 mL of DIPEA and 2-(2,6-dioxopiperidin-3-yl)-5-fluoroisoindoline-1,3-dione (see WO 2017197056 for the synthetic method) (34 mg, 0.12 mmol) were added. The resulting mixture was warmed to 80° C. and stirred for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=15:1), to afford compound 13 (25 mg, two-step total yield from compound 13c: 42%).

¹H NMR (400 MHz, CDCl₃) δ 8.30 (d, 1H), 7.96 (br.s, 1H), 7.67 (d, 1H), 7.44 (d, 1H), 7.32 (d, 1H), 7.30-7.26 (m, 1H), 7.13-7.01 (m, 3H), 6.91-6.85 (m, 2H), 6.70 (d, 1H), 4.98-4.88 (m, 3H), 4.42 (t, 2H), 4.00-3.90 (m, 2H), 3.90-3.75 (m, 6H), 3.07-2.93 (m, 2H), 2.93-2.66 (m, 5H), 2.51-2.39 (m, 4H), 2.30-2.17 (m, 3H), 2.17-2.08 (m, 2H), 2.03-1.70 (m, 6H), 1.64 (s, 6H), 1.61-1.47 (m, 6H).

LCMS m/z=976.3 [M+1]⁺

### Example 14

### 3-chloro-2-(2-chloroethoxy)-5-(2-(4-((2-(4-((1-((1-(2-(2,6-dioxopiperidin-3-yl)-1,3-dioxoisoindolin-4-yl)piperidin-4-yl)methyl)piperidin-4-yl)methyl)piperazin-1-yl)pyrimidin-4-yl)methoxy)phenyl)propan-2-yl)benzonitrile (Compound 14)

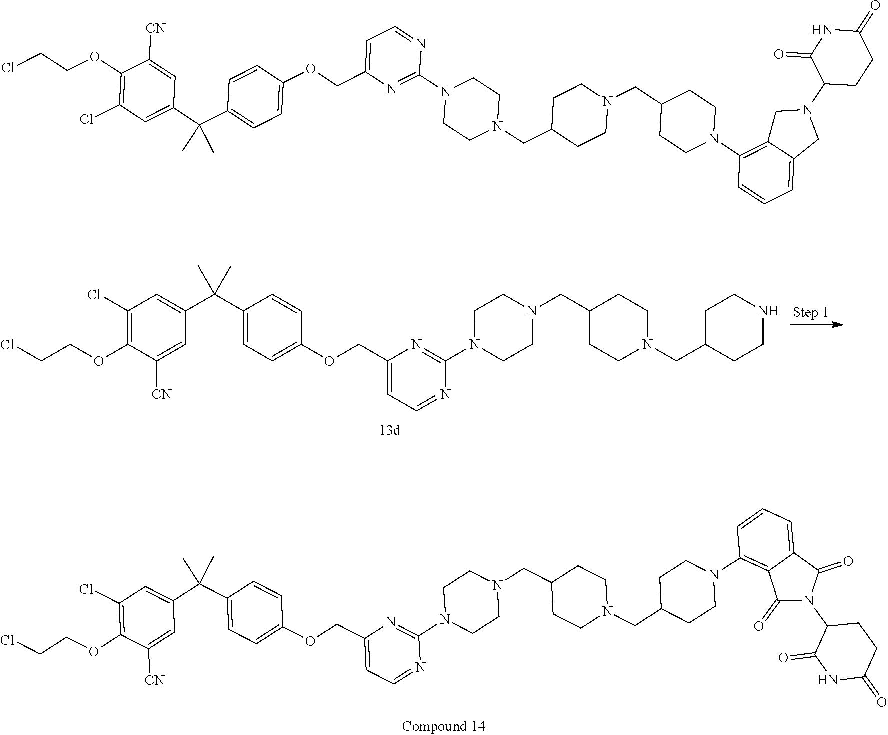 [SMILES: CC(C)(c1ccc(OCc2ccnc(N3CCN(CC4CCN(CC5CCN(c6cccc7c6CN(C6CCC(=O)NC6=O)C7)CC5)CC4)CC3)n2)cc1)c1cc(Cl)c(OCCCl)c(C#N)c1 ; CC(C)(c1ccc(OCc2ccnc(N3CCN(CC4CCN(CC5CCNCC5)CC4)CC3)n2)cc1)c1cc(Cl)c(OCCCl)c(C#N)c1 ; CC(C)(c1ccc(OCc2ccnc(N3CCN(CC4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

The above crude 13d trifluoroacetate (60 mg) was dissolved in 4 mL of DMSO, and solid sodium bicarbonate (26 mg, 0.31 mmol) was added. The mixture was stirred at room temperature for 10 min, and then 0.5 mL of DIPEA and 2-(2,6-dioxopiperidin-3-yl)-4-fluoroisoindoline-1,3-dione (see WO 2017197056 for the synthetic method) (34 mg, 0.12 mmol) were added. The resulting mixture was warmed to 80° C. and stirred for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=15:1), to afford compound 14 (20 mg, two-step total yield from compound 13c: 34%).

¹H NMR (400 MHz, CDCl₃) δ 8.30 (d, 1H), 7.99 (br.s, 1H), 7.60-7.51 (m, 1H), 7.44 (d, 1H), 7.36 (d, 1H), 7.32 (d, 1H), 7.17 (d, 1H), 7.13-7.04 (m, 2H), 6.93-6.84 (m, 2H), 6.70 (d, 1H), 5.02-4.90 (m, 3H), 4.42 (t, 2H), 3.92-3.68 (m, 8H), 3.00-2.65 (m, 7H), 2.52-2.40 (m, 4H), 2.40-2.17 (m, 4H), 2.17-2.06 (m, 1H), 2.04-1.86 (m, 3H), 1.86-1.70 (m, 3H), 1.70-1.42 (m, 12H).

LCMS m/z=976.3 [M+1]⁺

### Example 15

### 3-chloro-2-(2-chloroethoxy)-5-(2-(4-((2-(1′-((1-(2-(2,6-dioxopiperidin-3-yl)-1,3-dioxoisoindolin-4-yl)piperidin-4-yl)methyl)-[4,4′-bipiperidin]-1-yl)pyrimidin-4-yl)methoxy)phenyl)propan-2-yl)benzonitrile (Compound 15) trifluoroacetate

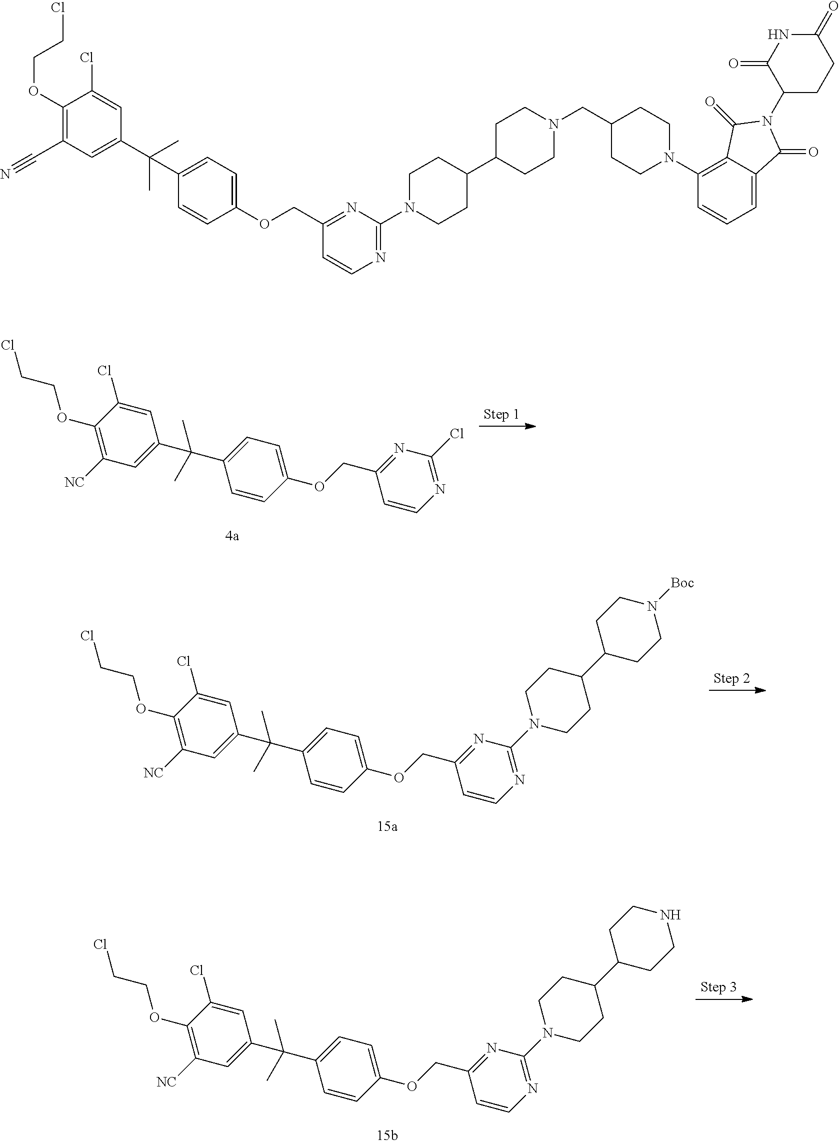 [SMILES: CC(C)(c1ccc(OCc2ccnc(N3CCC(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1 ; CC(C)(c1ccc(OCc2ccnc(Cl)n2)cc1)c1cc(Cl)c(OCCCl)c(C#N)c1 ; CC(C)(C)OC(=O)N1CCC(C2CCN(c3nccc(COc4ccc(C(C)(C)c5cc(Cl)c(OCCCl)c(C#N)c5)cc4)n3)CC2)CC1 ; CC(C)(c1ccc(OCc2ccnc(N3CCC(C4CCNCC4)CC3)n2)cc1)c1cc(Cl)c(OCCCl)c(C#N)c1]

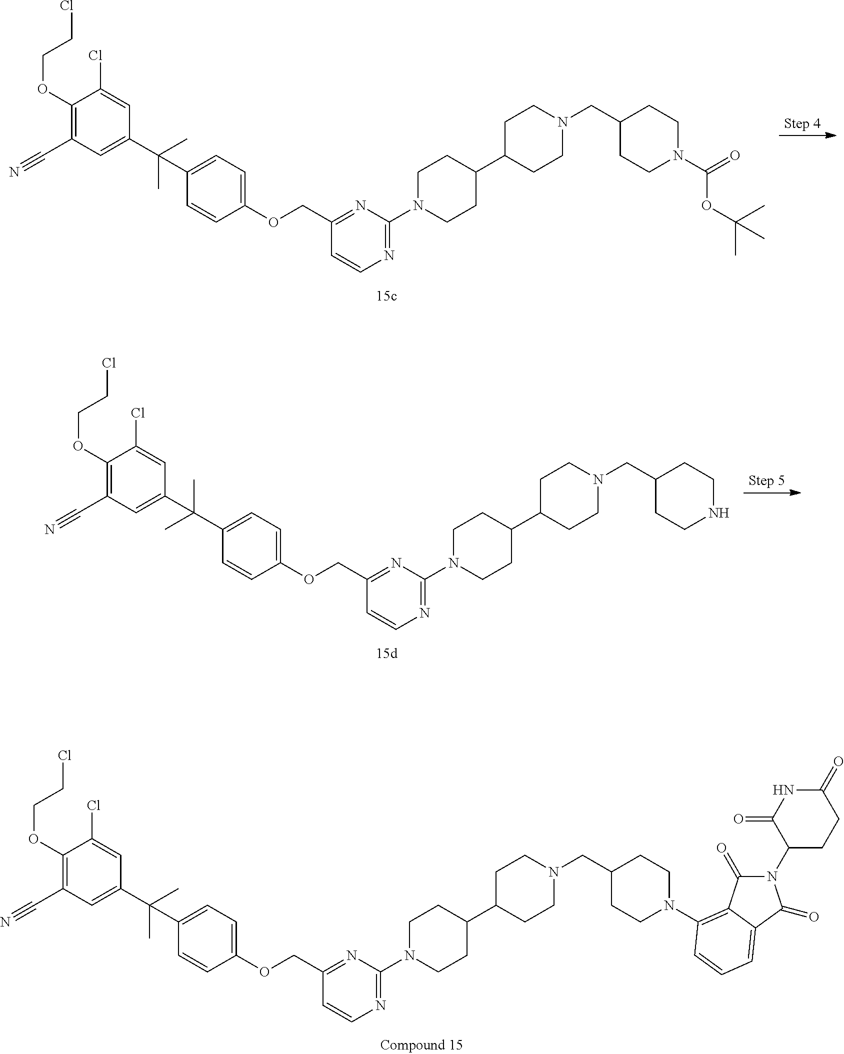 [SMILES: CC(C)(C)OC(=O)N1CCC(CN2CCC(C3CCN(c4nccc(COc5ccc(C(C)(C)c6cc(Cl)c(OCCCl)c(C#N)c6)cc5)n4)CC3)CC2)CC1 ; CC(C)(c1ccc(OCc2ccnc(N3CCC(C4CCN(CC5CCNCC5)CC4)CC3)n2)cc1)c1cc(Cl)c(OCCCl)c(C#N)c1 ; CC(C)(c1ccc(OCc2ccnc(N3CCC(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

### Step 1: tert-butyl 1′-(4-((4-(2-(3-chloro-4-(2-chloroethoxy)-5-cyanophenyl)propan-2-yl)phenoxy)methyl)pyrimidin-2-yl)-[4,4′-bipiperidine]-1-carboxylate (15a)

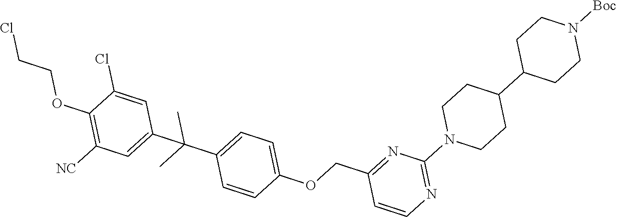 [SMILES: CC(C)(C)OC(=O)N1CCC(C2CCN(c3nccc(COc4ccc(C(C)(C)c5cc(Cl)c(OCCCl)c(C#N)c5)cc4)n3)CC2)CC1]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (1.70 g, 3.57 mmol) was dissolved in 60 mL of N,N′-dimethylformamide, and tert-butyl [4,4′-bipiperidine]-1-carboxylate (1.41 g, 5.25 mmol) and potassium bicarbonate (0.70 mg, 7.0 mmol) were successively added. The mixture was warmed to 90° C. and stirred for 2 h. The reaction liquid was cooled to room temperature, and to the reaction liquid was slowly added 100 mL of water. The mixture was extracted with 120 mL of ethyl acetate, and the organic phase was washed with 50 mL of saturated sodium chloride solution, dried over anhydrous sodium sulfate, and concentrated under reduced pressure, to afford 15a (1.98 g, yield: 78%).

LCMS m/z=708.2 [M+1]⁺

### Step 2: 5-(2-(4-((2-([4,4′-bipiperidin]-1-yl)pyrimidin-4-yl)methoxy)phenyl)propan-2-yl)-3-chloro-2-(2-chloroethoxy)benzonitrile (15b)

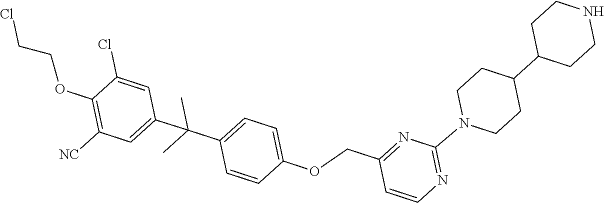 [SMILES: CC(C)(c1ccc(OCc2ccnc(N3CCC(C4CCNCC4)CC3)n2)cc1)c1cc(Cl)c(OCCCl)c(C#N)c1]

15a (1.98 g, 2.80 mmol) was dissolved in 5 mL of DCM, and 5 mL of trifluoroacetic acid was added. The mixture was stirred at room temperature for 4 h. The reaction liquid was concentrated under reduced pressure, and the residue was dissolved with 50 mL of DCM, and adjusted to pH 9 with a saturated sodium bicarbonate solution. Liquid separation was performed. The aqueous phase was extracted with 100 mL of dichloromethane, and the organic phases were combined, dried over anhydrous sodium sulfate, and concentrated under reduced pressure, to afford crude 15b (1.5 g).

LCMS m/z=608.3 [M+1]⁺

### Step 3: tert-butyl 4-((1′-(4-((4-(2-(3-chloro-4-(2-chloroethoxy)-5-cyanophenyl)propan-2-yl)phenoxy)methyl)pyrimidin-2-yl)-[4,4′-bipiperidin]-1-yl)methyl)piperidine-1-carboxylate (15c)

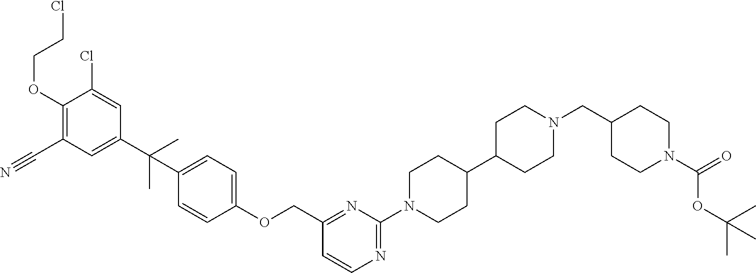 [SMILES: CC(C)(C)OC(=O)N1CCC(CN2CCC(C3CCN(c4nccc(COc5ccc(C(C)(C)c6cc(Cl)c(OCCCl)c(C#N)c6)cc5)n4)CC3)CC2)CC1]

The above crude 15b (150 mg) was dissolved in 15 mL of DCE, and tert-butyl 4-formylpiperidine-1-carboxylate (106.5 mg, 0.5 mmol) was added. The mixture was stirred at room temperature for 60 min, and then sodium triacetoxyborohydride (160 mg, 0.75 mmol) was added. The resulting mixture was reacted at room temperature for 16 h. To the reaction system was slowly added 30 mL of saturated sodium bicarbonate solution, and the mixture was extracted with 120 mL of dichloromethane. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 15c (120 mg, two-step yield from compound 15a: 53%).

LCMS m/z=805.3 [M+1]⁺

### Step 4: 3-chloro-2-(2-chloroethoxy)-5-(2-(4-((2-(1′-(piperidin-4-ylmethyl)-[4,4′-bipiperidin]-1-yl)pyrimidin-4-yl)methoxy)phenyl)propan-2-yl)benzonitrile (15d)

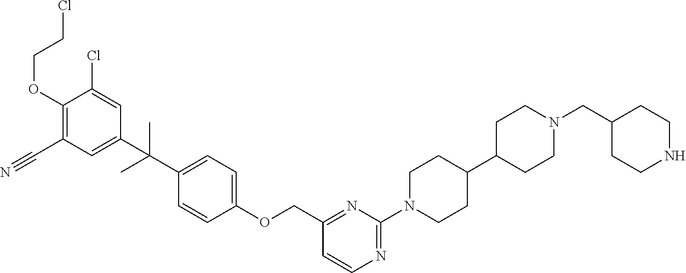 [SMILES: CC(C)(c1ccc(OCc2ccnc(N3CCC(C4CCN(CC5CCNCC5)CC4)CC3)n2)cc1)c1cc(Cl)c(OCCCl)c(C#N)c1]

15c (120 mg, 0.15 mmol) was dissolved in 2 mL of DCM, and 1 mL of trifluoroacetic acid was added. The mixture was stirred at room temperature for 4 h. The reaction liquid was concentrated under reduced pressure, and the residue was dissolved with 50 mL of DCM, and adjusted to pH 9 with a saturated sodium bicarbonate solution. Liquid separation was performed. The aqueous phase was extracted with 100 mL of dichloromethane, and the organic phases were combined, dried over anhydrous sodium sulfate, and concentrated under reduced pressure, to afford crude 15d (80 mg).

LCMS m/z=705.3 [M+1]⁺

### Step 5: 3-chloro-2-(2-chloroethoxy)-5-(2-(4-((2-(1′-((1-(2-(2,6-dioxopiperidin-3-yl)-1,3-dioxoisoindolin-4-yl)piperidin-4-yl)methyl)-[4,4′-bipiperidin]-1-yl)pyrimidin-4-yl)methoxy)phenyl)propan-2-yl)benzonitrile (Compound 15) trifluoroacetate

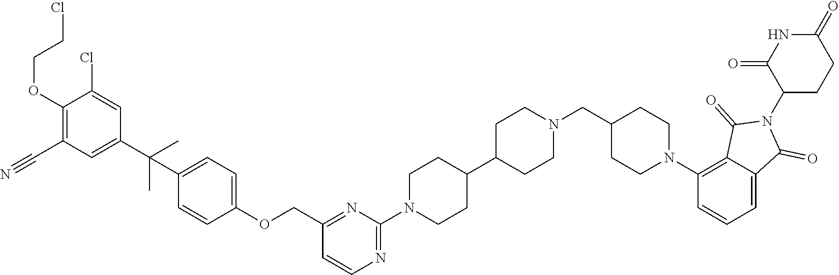 [SMILES: CC(C)(c1ccc(OCc2ccnc(N3CCC(C4CCN(CC5CCN(c6cccc7c6C(=O)N(C6CCC(=O)NC6=O)C7=O)CC5)CC4)CC3)n2)cc1)c1cc(Cl)c(OCCCl)c(C#N)c1]

The above crude 15d (80 mg) was dissolved in 5 mL of DMSO, and 0.2 mL of DIPEA and 2-(2,6-dioxopiperidin-3-yl)-4-fluoroisoindoline-1,3-dione (see WO 2017197056 for the synthetic method) (33 mg, 0.12 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 15 trifluoroacetate (10 mg).

¹H NMR (400 MHz, CDCl₃) δ 8.29 (d, 1H), 8.04 (br.s, 1H), 7.60-7.51 (m, 1H), 7.43 (d, 1H), 7.38-7.30 (m, 2H), 7.16 (d, 1H), 7.12-7.05 (m, 2H), 6.92-6.85 (m, 2H), 6.65 (d, 1H), 5.00-4.89 (m, 3H), 4.87-4.76 (m, 2H), 4.41 (t, 2H), 3.87 (t, 2H), 3.80-3.67 (m, 2H), 3.05-2.60 (m, 9H), 2.32-2.06 (m, 3H), 2.00-1.84 (m, 3H), 1.83-1.75 (m, 3H), 1.74-1.66 (m, 2H), 1.63 (s, 6H), 1.55-1.44 (m, 2H), 1.30-1.18 (m, 4H), 1.18-1.09 (m, 1H), 0.93-0.87 (m, 2H).

LCMS m/z=481.3 [M/2+1]⁺

### Example 16

### 3-chloro-2-(2-chloroethoxy)-5-(2-(4-((2-((3aR,6aS)-5-(1-((1-(2-(2,6-dioxopiperidin-3-yl)-1,3-dioxoisoindolin-5-yl)piperidin-4-yl)methyl)piperidin-4-yl)hexahydropyrrolo[3,4-c]pyrrol-2(1H)-yl)pyrimidin-4-yl)methoxy)phenyl)propan-2-yl)benzonitrile (Compound 16)

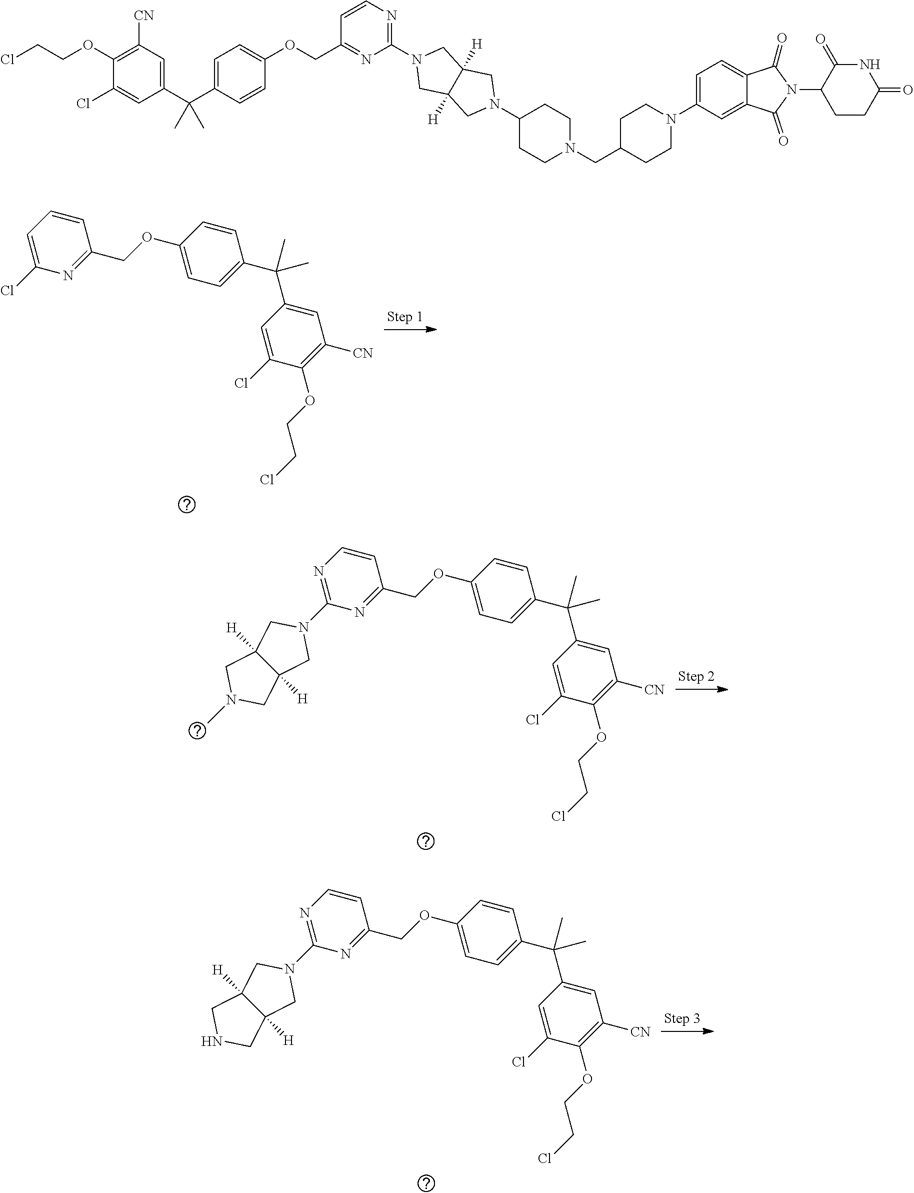 [SMILES: CC(C)(c1ccc(OCc2ccnc(N3C[C@@H]4CN(C5CCN(CC6CCN(c7ccc8c(c7)C(=O)N(C7CCC(=O)NC7=O)C8=O)CC6)CC5)C[C@@H]4C3)n2)cc1)c1cc(Cl)c(OCCCl)c(C#N)c1 ; CC(C)(c1ccc(OCc2cccc(Cl)n2)cc1)c1cc(Cl)c(OCCCl)c(C#N)c1 ; CN1C[C@@H]2CN(c3nccc(COc4ccc(C(C)(C)c5cc(Cl)c(OCCCl)c(C#N)c5)cc4)n3)C[C@@H]2C1 ; CC(C)(c1ccc(OCc2ccnc(N3C[C@H]4CNC[C@H]4C3)n2)cc1)c1cc(Cl)c(OCCCl)c(C#N)c1]

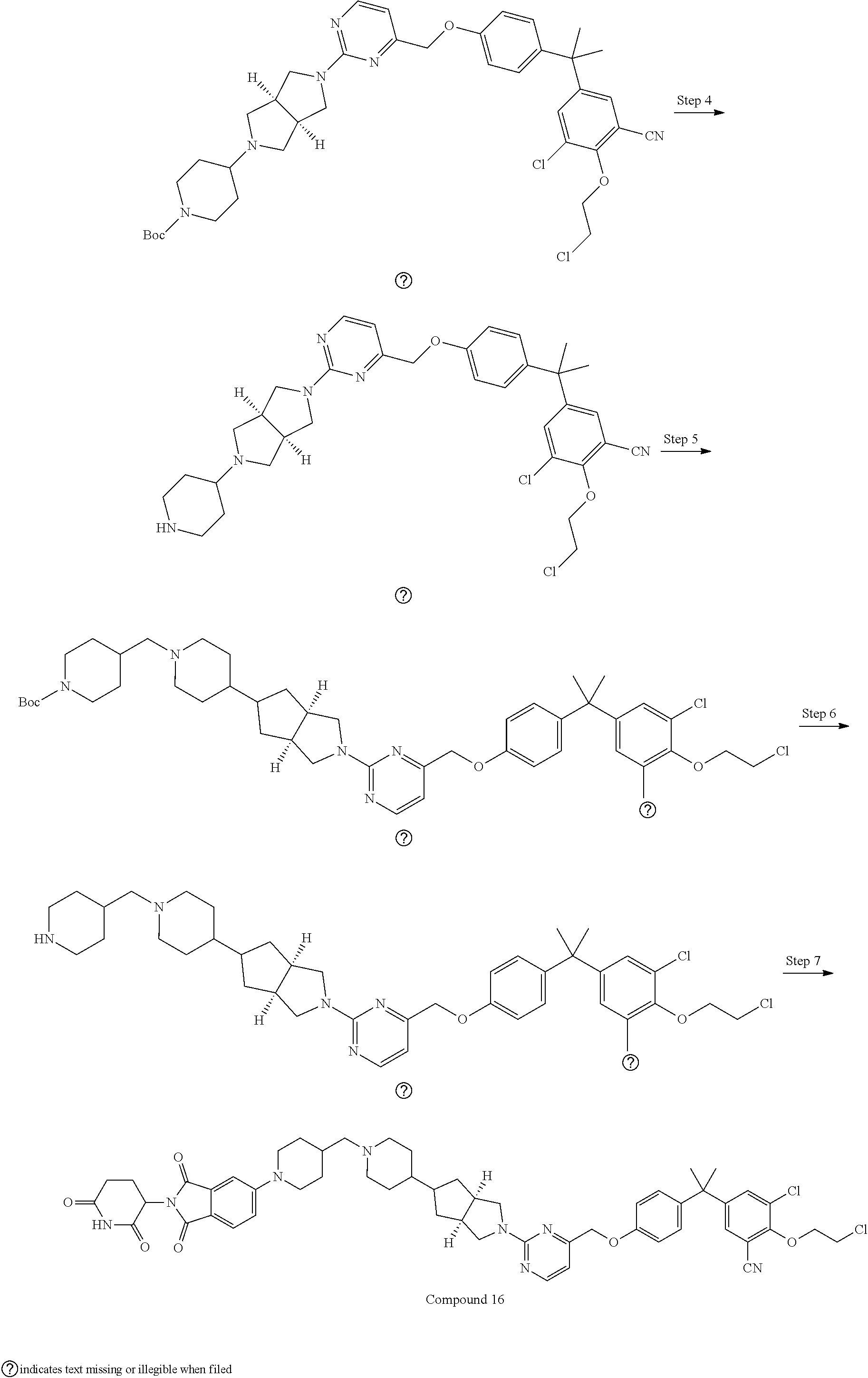

### Step 1: tert-butyl (3aR,6aS)-5-(4-((4-(2-(3-chloro-4-(2-chloroethoxy)-5-cyanophenyl) propan-2-yl)phenoxy)methyl)pyrimidin-2-yl)hexahydropyrrolo[3,4-c]pyrrole-2(1H)-carboxylate (16a)

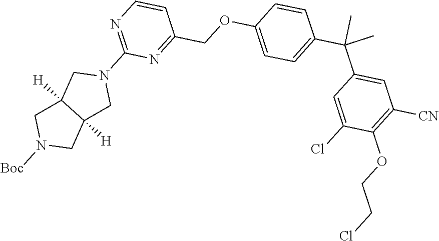 [SMILES: CC(C)(C)OC(=O)N1C[C@@H]2CN(c3nccc(COc4ccc(C(C)(C)c5cc(Cl)c(OCCCl)c(C#N)c5)cc4)n3)C[C@@H]2C1]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (300 mg, 0.63 mmol) and tert-butyl (3aR,6aS)-hexahydropyrrolo[3,4-c]pyrrole-2(1H)-carboxylate (130 mg, 0.61 mmol) were dissolved in 15 mL of DMF, and potassium bicarbonate (130 mg, 1.30 mmol) was added at room temperature. The mixture was warmed to 60° C. and reacted for 3 h. The reaction liquid was cooled to room temperature and filtered. To the filtrate was added 20 mL of water, and the mixture was extracted with 30 mL of ethyl acetate, and washed with a saturated sodium chloride solution (15 mL×2). The organic phase was collected, and dried over anhydrous sodium sulfate. The crude product was separated and purified by silica gel column chromatography (petroleum ether/ethyl acetate (v/v)=10:1-5:1), to afford 16a (0.31 g, yield: 75%).

### Step 2: 3-chloro-2-(2-chloroethoxy)-5-(2-(4-((2-((3aR,6aS)-hexahydropyrrolo[3,4-c]pyrrol-2(1H)-yl)pyrimidin-4-yl)methoxy)phenyl)propan-2-yl)benzonitrile (16b)

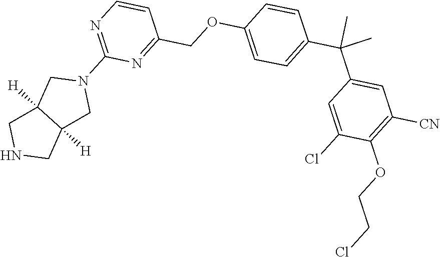 [SMILES: CC(C)(c1ccc(OCc2ccnc(N3C[C@H]4CNC[C@H]4C3)n2)cc1)c1cc(Cl)c(OCCCl)c(C#N)c1]

16a (148 mg, 0.23 mmol) was dissolved in 4 mL of DCM, and 2 mL of trifluoroacetic acid was added. The mixture was reacted at room temperature for 2 h. The reaction liquid was concentrated under reduced pressure, and adjusted to pH 10 with a saturated aqueous sodium bicarbonate solution. The aqueous phase was extracted with dichloromethane (10 mL×3), and the organic phases were combined, dried over anhydrous sodium sulfate, and concentrated under reduced pressure, to afford crude 16b (120 mg).

### Step 3: tert-butyl 4-((3aR,6aS)-5-(4-((4-(2-(3-chloro-4-(2-chloroethoxy)-5-cyanophenyl) propan-2-yl)phenoxy)methyl)pyrimidin-2-yl)hexahydropyrrolo[3,4-c]pyrrol-2(1H)-yl)piperidine-1-carboxylate (16c)

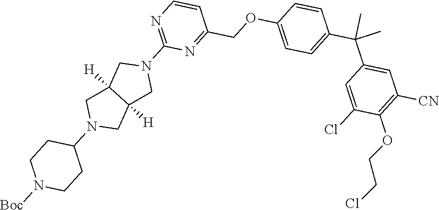 [SMILES: CC(C)(C)OC(=O)N1CCC(N2C[C@H]3CN(c4nccc(COc5ccc(C(C)(C)c6cc(Cl)c(OCCCl)c(C#N)c6)cc5)n4)C[C@H]3C2)CC1]

The above crude 16b (120 mg) and N-Boc-piperidone (88 mg, 0.44 mmol) were dissolved in 8 mL of trichloromethane, and acetic acid (40 mg, 0.66 mmol) and anhydrous magnesium sulfate (100 mg) were successively added. The mixture was warmed to 70° C. and reacted for 3 h. The mixture was cooled to room temperature, and sodium triacetoxyborohydride (230 mg, 1.09 mmol) was added. The resulting mixture was reacted at room temperature for 16 h. To the reaction liquid was added 20 mL of water, and the mixture was adjusted to pH 10 with a saturated aqueous sodium bicarbonate solution, and extracted with dichloromethane (30 mL×3). The organic phases were combined, washed with water (20 mL×2), dried over anhydrous sodium sulfate, and concentrated. The crude product was separated and purified by silica gel column chromatography (dichloromethane/methanol (v/v)=20:1-8:1), to afford 16c (102 mg, two-step yield from compound 16a: 60%).

Step 4: 3-chloro-2-(2-chloroethoxy)-5-(2-(4-((2-((3aR,6aS)-5-(piperidin-4-yl)hexahydropyrrolo[3,4-c]pyrrol-2(1H)-yl)pyrimidin-4-yl)methoxy)phenyl)propan-2-yl)benzonitrile (16d)

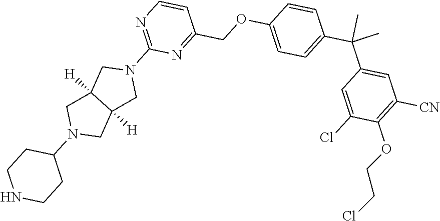 [SMILES: CC(C)(c1ccc(OCc2ccnc(N3C[C@@H]4CN(C5CCNCC5)C[C@@H]4C3)n2)cc1)c1cc(Cl)c(OCCCl)c(C#N)c1]

16c (102 mg, 0.14 mmol) was dissolved in 3 mL of DCM, and 1.5 mL of trifluoroacetic acid was added. The mixture was reacted at room temperature for 2 h. The reaction liquid was concentrated under reduced pressure, and adjusted to pH 10 with a saturated aqueous sodium bicarbonate solution. The aqueous phase was extracted with dichloromethane (10 mL×3), and the organic phases were combined, dried over anhydrous sodium sulfate, and concentrated under reduced pressure, to afford crude 16d (85 mg).

### Step 5: tert-butyl 4-((4-((3aR,6aS)-5-(4-((4-(2-(3-chloro-4-(2-chloroethoxy)-5-cyanophenyl)propan-2-yl)phenoxy)methyl)pyrimidin-2-yl)hexahydropyrrolo[3,4-c]pyrrol-2(1H)-yl)piperidin-1-yl)methyl)piperidine-1-carboxylate (16e)

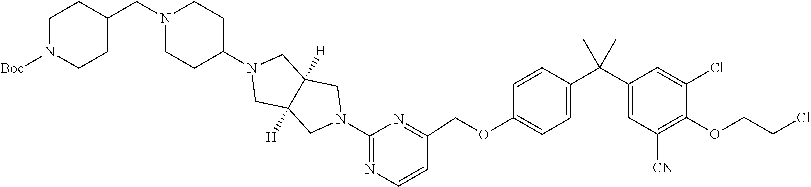 [SMILES: CC(C)(C)OC(=O)N1CCC(CN2CCC(N3C[C@H]4CN(c5nccc(COc6ccc(C(C)(C)c7cc(Cl)c(OCCCl)c(C#N)c7)cc6)n5)C[C@H]4C3)CC2)CC1]

The above crude 16d (85 mg) was dissolved in 8 mL of trichloromethane, and acetic acid (10 mg, 0.167 mmol) and anhydrous magnesium sulfate (80 mg) were added. The mixture was warmed to 70° C. and reacted for 3 h. The mixture was cooled to room temperature, and sodium triacetoxyborohydride (140 mg, 0.66 mmol) was added. The resulting mixture was reacted at room temperature for 16 h. To the reaction liquid was added 20 mL of water, and the mixture was adjusted to pH 10 with a saturated aqueous sodium bicarbonate solution, and extracted with dichloromethane (30 mL×3). The organic phases were combined, washed with water (20 mL×2), dried over anhydrous sodium sulfate, and concentrated under reduced pressure. The crude product was separated and purified by silica gel column chromatography (dichloromethane/methanol (v/v)=20:1-8:1), to afford 16e (51 mg, two-step yield from compound 16c: 44%).

### Step 6: 3-chloro-2-(2-chloroethoxy)-5-(2-(4-((2-((3aR,6aS)-5-(1-(piperidin-4-ylmethyl)piperidin-4-yl)hexahydropyrrolo[3,4-c]pyrrol-2(1H)-yl)pyrimidin-4-yl)methoxy)phenyl)propan-2-yl)benzonitrile (16f)

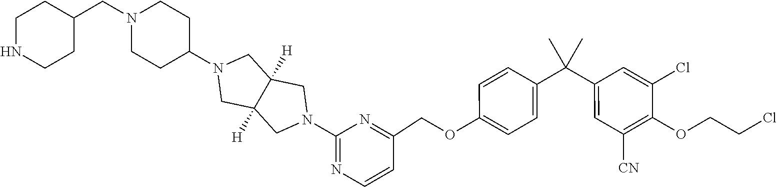 [SMILES: CC(C)(c1ccc(OCc2ccnc(N3C[C@@H]4CN(C5CCN(CC6CCNCC6)CC5)C[C@@H]4C3)n2)cc1)c1cc(Cl)c(OCCCl)c(C#N)c1]

16e (51 mg, 0.061 mmol) was dissolved in 3 mL of DCM, and 1 mL of trifluoroacetic acid was added. The mixture was reacted at room temperature for 2.5 h. The reaction liquid was adjusted to pH 10 with a saturated aqueous sodium bicarbonate solution. The aqueous phase was extracted with dichloromethane (10 mL×3), and the organic phases were combined, dried over anhydrous sodium sulfate, and concentrated under reduced pressure, to afford crude 16f (49 mg).

### Step 7: 3-chloro-2-(2-chloroethoxy)-5-(2-(4-((2-((3aR,6aS)-5-(1-((1-(2-(2,6-dioxopiperidin-3-yl)-1,3-dioxoisoindolin-5-yl)piperidin-4-yl)methyl)piperidin-4-yl)hexahydropyrrolo[3,4-c]pyrrol-2(1H)-yl)pyrimidin-4-yl)methoxy)phenyl)propan-2-yl)benzonitrile (Compound 16)

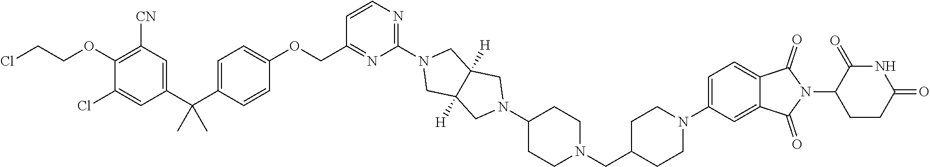 [SMILES: CC(C)(c1ccc(OCc2ccnc(N3C[C@@H]4CN(C5CCN(CC6CCN(c7ccc8c(c7)C(=O)N(C7CCC(=O)NC7=O)C8=O)CC6)CC5)C[C@@H]4C3)n2)cc1)c1cc(Cl)c(OCCCl)c(C#N)c1]

The above crude 16f (49 mg) was dissolved in 8 mL of DMSO, and 2-(2,6-dioxopiperidin-3-yl)-5-fluoroisoindoline-1,3-dione (see WO 2017197056 for the synthetic method) (19 mg, 0.069 mmol) and diisopropylethylamine (43 mg, 0.33 mmol) were added at room temperature. The mixture was warmed to 80° C. and reacted for 3.5 h. The mixture was cooled to room temperature, and the reaction liquid was poured into 30 mL of ethyl acetate, and washed with a saturated aqueous sodium chloride solution (30 mL×3). The organic phase was dried over anhydrous sodium sulfate, and concentrated under reduced pressure. The crude product was then separated and purified by silica gel column chromatography (dichloromethane/methanol (v/v)=15:1-8:1), to afford compound 16 (51 mg, two-step yield from compound 16e: 84%).

¹H NMR (400 MHz, CDCl₃) δ 8.36-8.20 (m, 2H), 7.66 (d, 1H), 7.44 (d, 1H), 7.32 (d, 1H), 7.29-7.26 (m, 1H), 7.13-6.99 (m, 3H), 6.93-6.86 (m, 2H), 6.73 (d, 1H), 5.00-4.88 (m, 3H), 4.41 (t, 2H), 3.98-3.82 (m, 4H), 3.80-3.68 (m, 2H), 3.63-3.50 (m, 2H), 3.10-2.65 (m, 11H), 2.55-2.43 (m, 2H), 2.28-2.07 (m, 4H), 2.06-1.93 (m, 2H), 1.93-1.81 (m, 4H), 1.81-1.72 (m, 1H), 1.70-1.52 (m, 8H), 1.30-1.24 (m, 2H).

LCMS m/z=988.3 [M+1]⁺

### Example 17: 3-chloro-2-(2-chloroethoxy)-5-(2-(4-((2-((3aR,6aS)-5-(1-(1-(2-(2,6-dioxopiperidin-3-yl)-1,3-dioxoisoindolin-5-yl)azetidin-3-yl)piperidin-4-yl)hexahydropyrrolo[3,4-c]pyrrol-2(1H)-yl)pyrimidin-4-yl)methoxy)phenyl)propan-2-yl)benzonitrile (Compound 17)

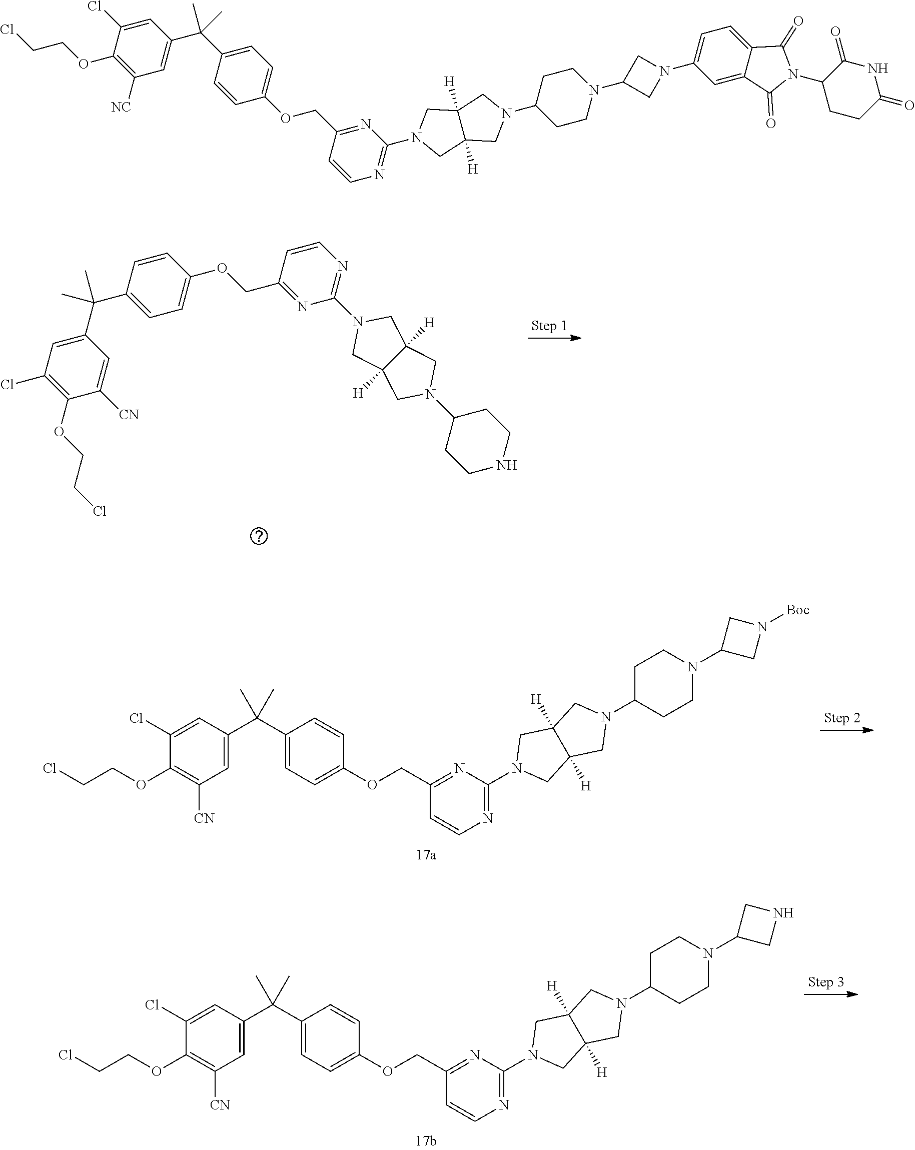 [SMILES: CC(C)(c1ccc(OCc2ccnc(N3C[C@@H]4CN(C5CCN(C6CN(c7ccc8c(c7)C(=O)N(C7CCC(=O)NC7=O)C8=O)C6)CC5)C[C@@H]4C3)n2)cc1)c1cc(Cl)c(OCCCl)c(C#N)c1 ; CC(C)(c1ccc(OCc2ccnc(N3C[C@@H]4CN(C5CCNCC5)C[C@@H]4C3)n2)cc1)c1cc(Cl)c(OCCCl)c(C#N)c1 ; CC(C)(C)OC(=O)N1CC(N2CCC(N3C[C@H]4CN(c5nccc(COc6ccc(C(C)(C)c7cc(Cl)c(OCCCl)c(C#N)c7)cc6)n5)C[C@H]4C3)CC2)C1 ; CC(C)(c1ccc(OCc2ccnc(N3C[C@@H]4CN(C5CCN(C6CNC6)CC5)C[C@@H]4C3)n2)cc1)c1cc(Cl)c(OCCCl)c(C#N)c1]

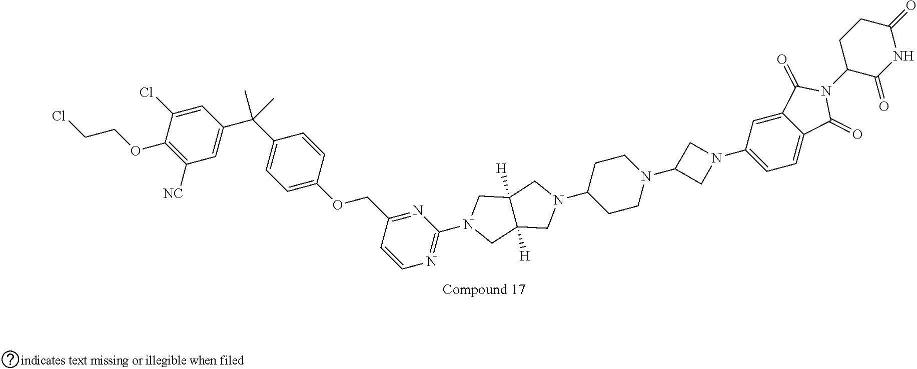 [SMILES: CC(C)(c1ccc(OCc2ccnc(N3C[C@@H]4CN(C5CCN(C6CN(c7ccc8c(c7)C(=O)N(C7CCC(=O)NC7=O)C8=O)C6)CC5)C[C@@H]4C3)n2)cc1)c1cc(Cl)c(OCCCl)c(C#N)c1]

### Step 1: tert-butyl 3-(4-((3aR,6aS)-5-(4-((4-(2-(3-chloro-4-(2-chloroethoxy)-5-cyanophenyl)propan-2-yl)phenoxy)methyl)pyrimidin-2-yl)hexahydropyrrolo[3,4-c]pyrrol-2(1H)-yl)piperidin-1-yl)azetidine-1-carboxylate (17a)

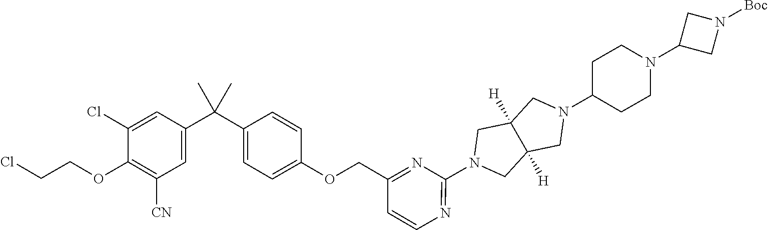 [SMILES: CC(C)(C)OC(=O)N1CC(N2CCC(N3C[C@H]4CN(c5nccc(COc6ccc(C(C)(C)c7cc(Cl)c(OCCCl)c(C#N)c7)cc6)n5)C[C@H]4C3)CC2)C1]

The above crude 16d (85 mg) was dissolved in 10 mL of trichloromethane, and acetic acid (11 mg, 0.183 mmol) and anhydrous magnesium sulfate (120 mg) were successively added. The mixture was warmed to 70° C. and reacted for 3 h. The mixture was cooled to room temperature, and sodium triacetoxyborohydride (141 mg, 0.67 mmol) was added. The resulting mixture was reacted at room temperature for 16 h. To the reaction liquid was added 20 mL of water, and the mixture was adjusted to pH 10 with a saturated aqueous sodium bicarbonate solution, and extracted with dichloromethane (30 mL×3). The organic phases were combined, washed with water (20 mL×2), dried over anhydrous sodium sulfate, and concentrated under reduced pressure. The crude product was separated and purified by silica gel column chromatography (dichloromethane/methanol (v/v)=20:1-8:1), to afford 17a (73 mg, two-step yield from compound 16c: 66%).

### Step 2: 5-(2-(4-((2-((3aR,6aS)-5-(1-(azetidin-3-yl)piperidin-4-yl)hexahydropyrrolo[3,4-c]pyrrol-2(1H)-yl)pyrimidin-4-yl)methoxy)phenyl)propan-2-yl)-3-chloro-2-(2-chloroethoxy)benzonitrile (17b)

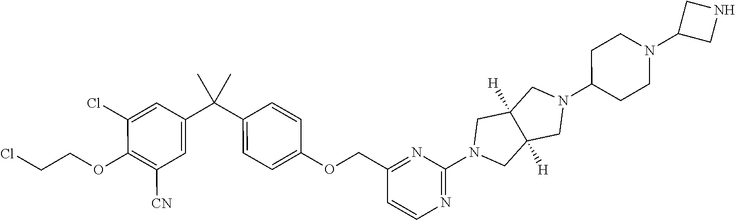 [SMILES: CC(C)(c1ccc(OCc2ccnc(N3C[C@@H]4CN(C5CCN(C6CNC6)CC5)C[C@@H]4C3)n2)cc1)c1cc(Cl)c(OCCCl)c(C#N)c1]

17a (73 mg, 0.092 mmol) was dissolved in 4 mL of DCM, and 1.5 mL of trifluoroacetic acid was added. The mixture was reacted at room temperature for 1.5 h. The reaction liquid was adjusted to pH 10 with a saturated aqueous sodium bicarbonate solution. The aqueous phase was extracted with dichloromethane (10 mL×3), and the organic phases were combined, dried over anhydrous sodium sulfate, and concentrated under reduced pressure, to afford crude 17b (60 mg).

### Step 3: 3-chloro-2-(2-chloroethoxy)-5-(2-(4-((2-((3aR,6aS)-5-(1-(1-(2-(2,6-dioxopiperidin-3-yl)-1,3-dioxoisoindolin-5-yl)azetidin-3-yl)piperidin-4-yl)hexahydropyrrolo[3,4-c]pyrrol-2(1H)-yl)pyrimidin-4-yl)methoxy)phenyl)propan-2-yl)benzonitrile (Compound 17)

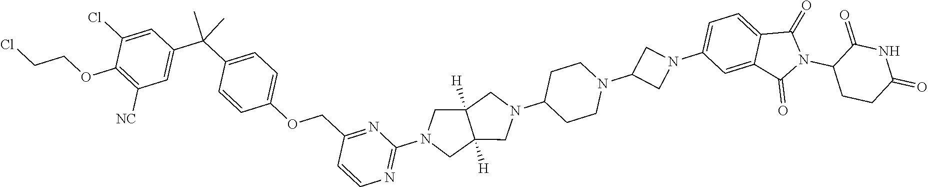 [SMILES: CC(C)(c1ccc(OCc2ccnc(N3C[C@@H]4CN(C5CCN(C6CN(c7ccc8c(c7)C(=O)N(C7CCC(=O)NC7=O)C8=O)C6)CC5)C[C@@H]4C3)n2)cc1)c1cc(Cl)c(OCCCl)c(C#N)c1]

The above crude 17b (60 mg) was dissolved in 5 mL of DMSO, and 2-(2,6-dioxopiperidin-3-yl)-5-fluoroisoindoline-1,3-dione (see WO 2017197056 for the synthetic method) (24 mg, 0.087 mmol) and diisopropylethylamine (56 mg, 0.43 mmol) were added at room temperature. The mixture was warmed to 80° C. and reacted for 3.5 h. The mixture was cooled to room temperature, and the reaction liquid was poured into 35 mL of ethyl acetate, and washed with a saturated aqueous sodium chloride solution (20 mL×3). The organic phase was dried over anhydrous sodium sulfate, and concentrated under reduced pressure. The crude product was separated and purified by silica gel column chromatography (dichloromethane/methanol (v/v)=15:1-8:1), to afford compound 17 (52 mg, two-step yield from compound 17a: 60%).

¹H NMR (400 MHz, CDCl₃) δ 8.32 (d, 1H), 8.00 (s, 1H), 7.63 (d, 1H), 7.44 (d, 1H), 7.31 (d, 1H), 7.13-7.04 (m, 2H), 6.93-6.84 (m, 2H), 6.81-6.70 (m, 2H), 6.51 (dd, 1H), 5.00-4.85 (m, 3H), 4.41 (t, 2H), 4.07 (t, 2H), 3.91-3.81 (m, 4H), 3.80-3.45 (m, 4H), 3.42-3.27 (m, 1H), 3.20-2.65 (m, 8H), 2.65-2.40 (m, 2H), 2.18-2.06 (m, 1H), 2.06-1.84 (m, 4H), 1.74-1.46 (m, 10H).

LCMS m/z=946.6 [M+1]⁺

### Example 18

### 3-chloro-5-(2-(4-((2-(4-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)piperazin-1-yl)pyrimidin-4-yl)methoxy)phenyl)propan-2-yl)-2-ethoxybenzonitrile (Compound 18)

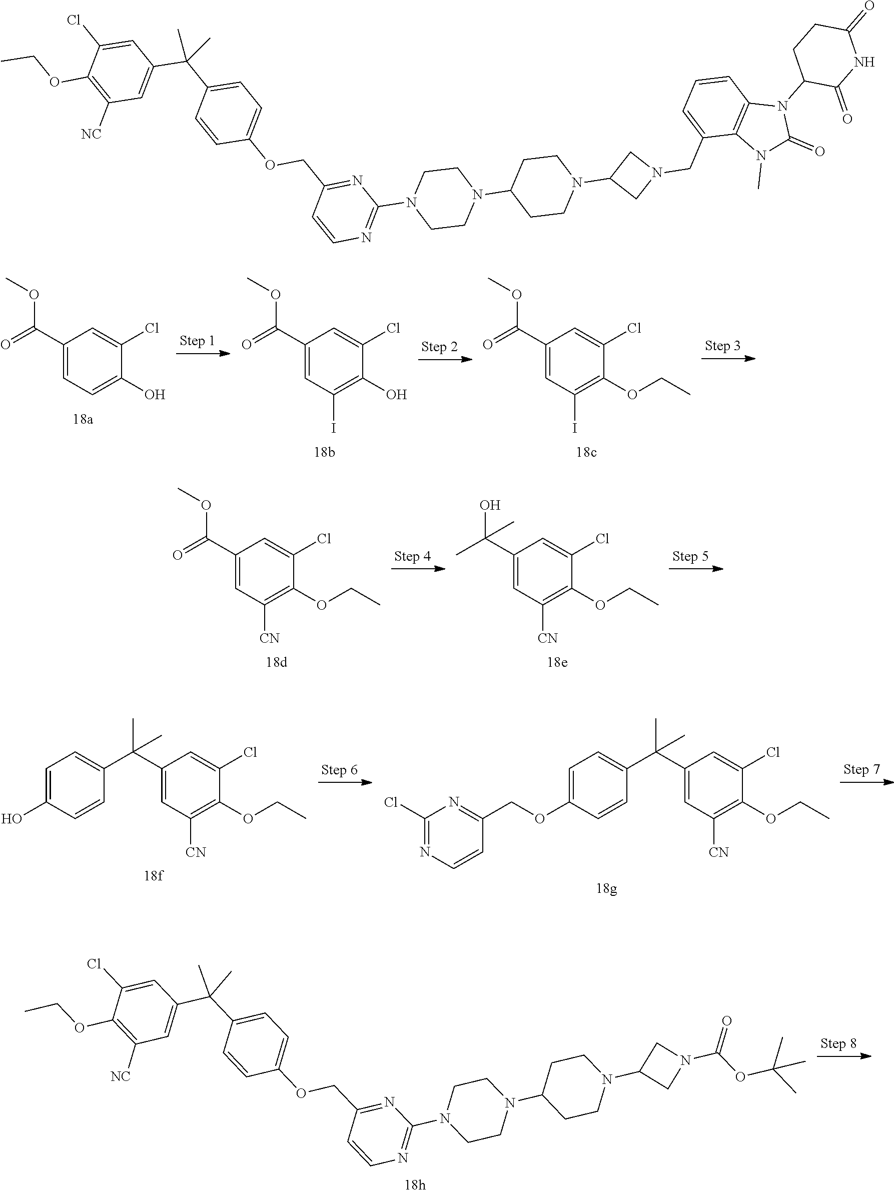

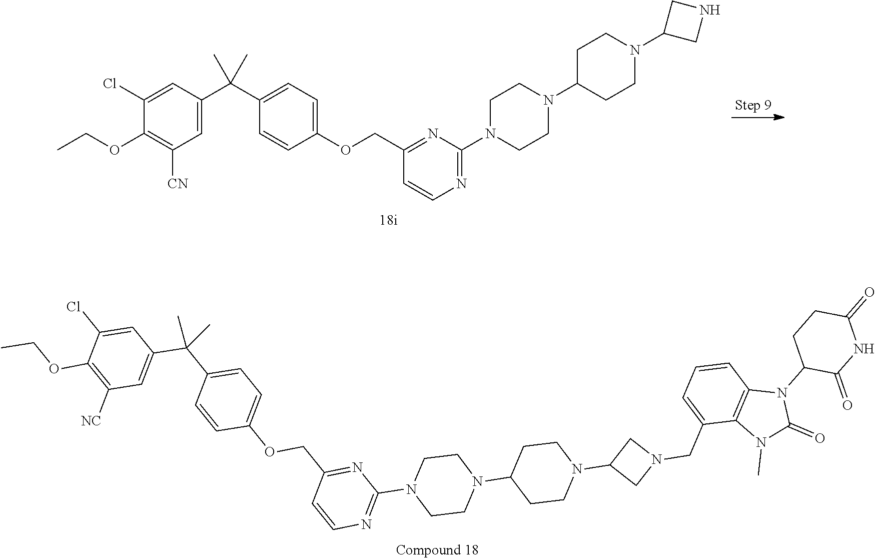 [SMILES: CCOc1c(Cl)cc(C(C)(C)c2ccc(OCc3ccnc(N4CCN(C5CCN(C6CNC6)CC5)CC4)n3)cc2)cc1C#N ; CCOc1c(Cl)cc(C(C)(C)c2ccc(OCc3ccnc(N4CCN(C5CCN(C6CN(Cc7cccc8c7n(C)c(=O)n8C7CCC(=O)NC7=O)C6)CC5)CC4)n3)cc2)cc1C#N]

### Step 1: methyl 3-chloro-4-hydroxy-5-iodobenzoate (18b)

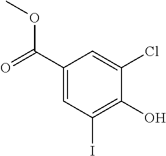

Methyl 3-chloro-4-hydroxybenzoate (18a) (8.0 g, 42.9 mmol) was dissolved in 100 mL of dichloromethane, and cooled to 0° C. N-iodosuccinimide (9.6 g, 42.7 mmol) was added, and the mixture was reacted at room temperature for 20 h. To the reaction liquid was added dropwise 100 mL of water, and layer separation was performed. The mixture was extracted with 50 mL of ethyl acetate, washed with 100 mL of saturated aqueous sodium thiosulfate solution, dried over anhydrous sodium sulfate, and concentrated under reduced pressure, to afford crude 18b (12 g).

### Step 2: methyl 3-chloro-4-ethoxy-5-iodobenzoate (18c)

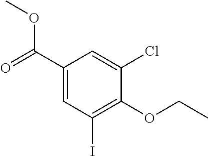

The above crude 18b (12 g) was dissolved in 100 mL of DMF, and iodoethane (9.0 g, 57.7 mmol) and potassium carbonate (10.66 g, 77.1 mmol) were added. The mixture was warmed to 60° C. and reacted for 2 h. The mixture was cooled to room temperature, and to the reaction liquid was slowly added 200 mL of water. The resulting mixture was extracted with 200 mL of ethyl acetate, and the organic phase was washed with a saturated sodium chloride solution (50 mL×2), dried over anhydrous sodium sulfate, and concentrated under reduced pressure, to afford 18c (9 g, two-step yield from compound 18a: 62%).

### Step 3: 3-chloro-2-ethoxy-5-(2-hydroxypropan-2-yl)benzonitrile (18d)

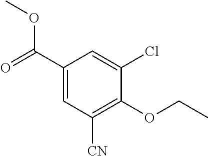 [SMILES: CCOc1c(Cl)cc(C(=O)OC)cc1C#N]

18c (9.0 g, 26.4 mmol) was dissolved in 100 mL of N-methylpyrrolidone, and copper cyanide (2.6 g, 29.03 mmol) was added. The mixture was warmed to 160° C. and reacted for 2 h. The reaction liquid was cooled to room temperature and filtered. To the filtrate was slowly added 100 mL of water, and the mixture was extracted with 200 mL of ethyl acetate. The organic phase was washed with a saturated sodium chloride solution (50 mL×2), dried over anhydrous sodium sulfate, and concentrated under reduced pressure, to afford 18d (5.0 g, yield: 79%).

### Step 4: 3-chloro-2-ethoxy-5-(2-hydroxypropan-2-yl)benzonitrile (18e)

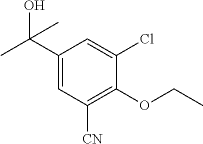 [SMILES: CCOc1c(Cl)cc(C(C)(C)O)cc1C#N]

8 mL of 3 mol/L solution of methylmagnesium bromide in 2-methyl tetrahydrofuran was added to 7 mL of tetrahydrofuran, and the mixture was cooled to 0° C. A solution of 18d (1.4 g, 5.84 mmol) in tetrahydrofuran (14 mL) was then added dropwise, and the mixture was reacted at 0° C. for further 2 h. To the reaction liquid was added dropwise 60 mL of saturated ammonium chloride solution, and the mixture was extracted with 50 mL of ethyl acetate, washed with 50 mL of saturated sodium chloride solution, dried over anhydrous sodium sulfate, and concentrated under reduced pressure, to afford crude 18e (1.3 g).

### Step 5: 3-chloro-2-ethoxy-5-(2-(4-hydroxyphenyl)propan-2-yl)benzonitrile (18f)

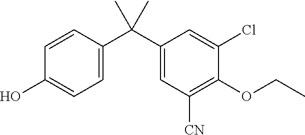 [SMILES: CCOc1c(Cl)cc(C(C)(C)c2ccc(O)cc2)cc1C#N]

The above crude 18e (1.3 g) was dissolved in 15 mL of carbon tetrachloride, and phenol (0.57 g, 6.06 mmol) was added. The mixture was cooled to 0° C., and boron trifluoride diethyl etherate (1.58 g, 11.13 mmol) was added. The resulting mixture was then warmed to room temperature and reacted for 3 h. To the reaction liquid was slowly added 20 mL of water, and the mixture was extracted with 30 mL of dichloromethane. The organic phase was washed with 50 mL of saturated sodium chloride solution, dried over anhydrous sodium sulfate, and concentrated under reduced pressure. The crude product was separated and purified by silica gel column chromatography (ethyl acetate/petroleum ether (v/v)=0:1-3:7), to afford 18f (1.50 g, two-step yield from compound 18d: 81%).

### Step 6: 3-chloro-5-(2-(4-((2-chloropyrimidin-4-yl)methoxy)phenyl)propan-2-yl)-2-ethoxybenzonitrile (18g)

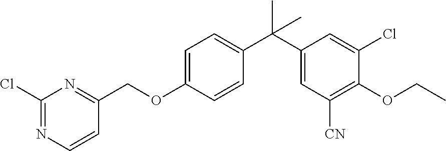 [SMILES: CCOc1c(Cl)cc(C(C)(C)c2ccc(OCc3ccnc(Cl)n3)cc2)cc1C#N]

18f (0.58 g, 1.84 mmol) was dissolved in 15 mL of acetonitrile, and 2-chloro-4-(chloromethyl)pyrimidine (0.30 g, 1.84 mmol) and potassium carbonate (0.51 g, 3.69 mmol) were successively added. The mixture was warmed to 70° C. and reacted for 20 h. The reaction liquid was cooled to room temperature, filtered, and concentrated under reduced pressure. The crude product was separated and purified by silica gel column chromatography (ethyl acetate/petroleum ether (v/v)=0:1-3:7), to afford 18g (0.38 g, yield: 47%).

LCMS m/z=442.3 [M+1]⁺

### Step 7: tert-butyl 3-(4-(4-(4-((4-(2-(3-chloro-5-cyano-4-ethoxyphenyl)propan-2-yl)phenoxy)methyl)pyrimidin-2-yl)piperazin-1-yl)piperidin-1-yl)azetidine-1-carboxylate (18h)

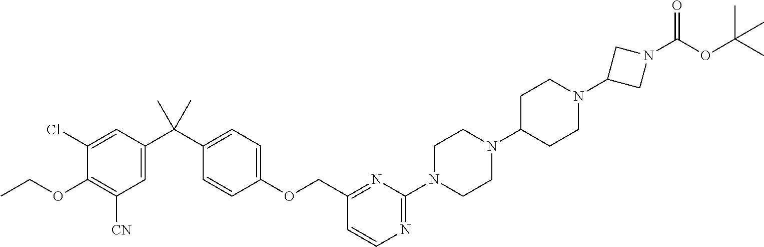 [SMILES: CCOc1c(Cl)cc(C(C)(C)c2ccc(OCc3ccnc(N4CCN(C5CCN(C6CN(C(=O)OC(C)(C)C)C6)CC5)CC4)n3)cc2)cc1C#N]

18g (270 mg, 0.61 mmol) was dissolved in 10 mL of DMF, and the above crude tert-butyl 3-(4-(piperazin-1-yl)piperidin-1-yl)azetidine-1-carboxylate (intermediate 1) (200 mg) and solid potassium bicarbonate (133 mg, 1.33 mmol) were successively added. The mixture was warmed to 90° C. and stirred for 4 h. The mixture was cooled to room temperature, and 30 mL of water was added. The resulting mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 18h (380 mg, yield from compound 18g: 85%).

LCMS m/z=730.5 [M+1]⁺

### Step 8: 5-(2-(4-((2-(4-(1-(azetidin-3-yl)piperidin-4-yl)piperazin-1-yl)pyrimidin-4-yl)methoxy)phenyl)propan-2-yl)-3-chloro-2-ethoxybenzonitrile (18i)

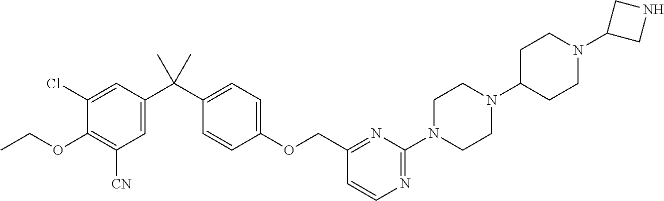 [SMILES: CCOc1c(Cl)cc(C(C)(C)c2ccc(OCc3ccnc(N4CCN(C5CCN(C6CNC6)CC5)CC4)n3)cc2)cc1C#N]

18h (380 mg, 0.52 mmol) was dissolved in 5 mL of DCM, and 2 mL of trifluoroacetic acid was added. The mixture was stirred at room temperature for 4 h. The reaction system was concentrated under reduced pressure, dissolved with 50 mL of DCM, adjusted to pH 10 with a saturated sodium bicarbonate solution, extracted with 50 mL of dichloromethane, dried over anhydrous sodium sulfate, and concentrated under reduced pressure, to afford crude 18i (300 mg).

LCMS m/z=630.3 [M+1]⁺

### Step 9: 3-chloro-5-(2-(4-((2-(4-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)piperazin-1-yl)pyrimidin-4-yl)methoxy)phenyl)propan-2-yl)-2-ethoxybenzonitrile (Compound 18)

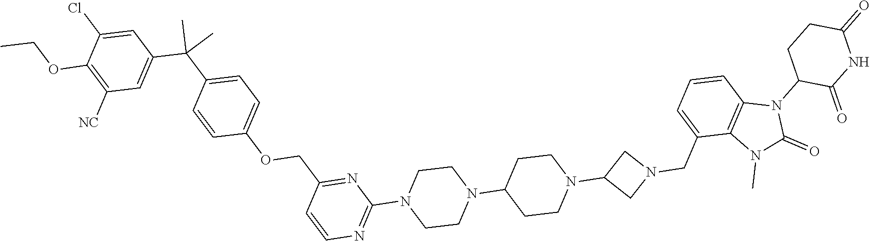 [SMILES: CCOc1c(Cl)cc(C(C)(C)c2ccc(OCc3ccnc(N4CCN(C5CCN(C6CN(Cc7cccc8c7n(C)c(=O)n8C7CCC(=O)NC7=O)C6)CC5)CC4)n3)cc2)cc1C#N]

The above crude 18i (100 mg) was dissolved in 10 mL of DMAc, and 1-(2,6-dioxopiperidin-3-yl)-3-methyl-2-oxo-2,3-dihydro-1H-benzo[d]imidazole-4-carbaldehyde (see WO 2020113233 for the synthetic method) (46 mg, 0.16 mmol), 0.1 mL of acetic acid and 1 g of molecular sieve powder were successively added. The mixture was warmed to 80° C. and reacted for 120 min, and sodium triacetoxyborohydride (230 mg, 1.085 mmol) was added. The mixture was cooled to room temperature, and to the reaction system was slowly added 30 mL of saturated sodium bicarbonate solution. The mixture was extracted twice with 60 mL of ethyl acetate, and the organic phase was washed with 50 mL of water, dried over anhydrous sodium sulfate, and concentrated under reduced pressure. The crude product was separated and purified by silica gel column chromatography (dichloromethane/methanol (v/v)=20:1-9:1), to afford compound 18 (10 mg, two-step yield from compound 18h: 6%).

¹H NMR (400 MHz, CDCl₃) δ 8.30-8.04 (m, 2H), 7.35 (s, 1H), 7.23 (s, 1H), 7.10-6.58 (m, 8H), 5.20-5.05 (m, 1H), 4.87 (s, 2H), 4.28-4.10 (m, 2H), 3.98-3.55 (m, 8H), 3.48-3.30 (m, 2H), 3.10-2.21 (m, 12H), 2.20-2.06 (m, 1H), 1.95-1.67 (m, 5H), 1.67-1.45 (m, 9H), 1.45-1.37 (m, 3H).

LCMS m/z=451.4 [M/2+1]⁺

### Example 19

### 3-chloro-2-(2-chloroethoxy)-5-(2-(4-((2-(4-((1-((1-(2-(2,6-dioxopiperidin-3-yl)-1,3-dioxoisoindolin-5-yl)azetidin-3-yl)methyl)piperidin-4-yl)methyl)piperazin-1-yl)pyrimidin-4-yl)methoxy)phenyl)propan-2-yl)benzonitrile (Compound 19) trifluoroacetate

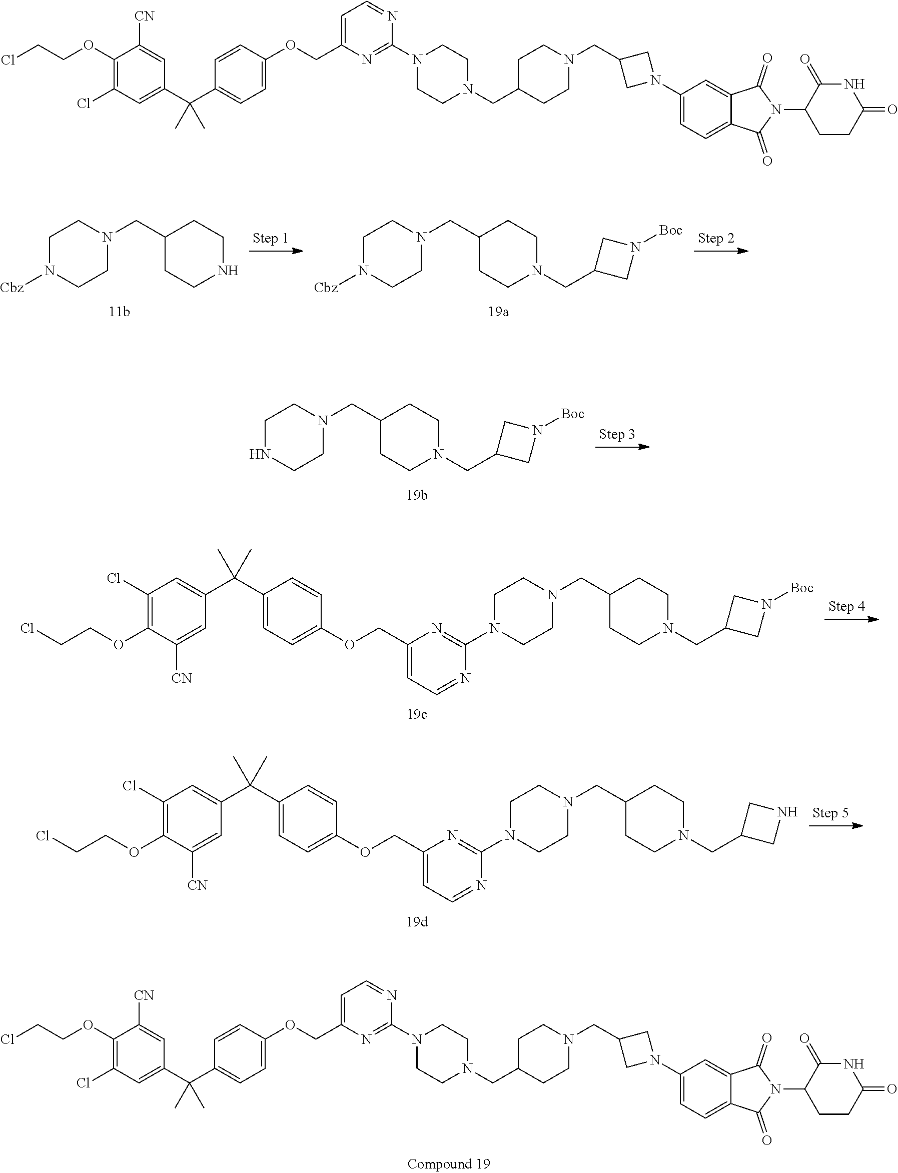

### Step 1: benzyl 4-((1-((1-(tert-butoxycarbonyl)azetidin-3-yl)methyl)piperidin-4-yl)methyl) piperazine-1-carboxylate (19a)

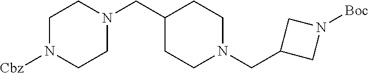 [SMILES: CC(C)(C)OC(=O)N1CC(CN2CCC(CN3CCN(C(=O)OCc4ccccc4)CC3)CC2)C1]

The crude benzyl 4-(piperidin-4-ylmethyl)piperazine-1-carboxylate (11b) (1.0 g) was dissolved in 15 mL of DCE, and tert-butyl 3-formylazetidine-1-carboxylate (875 mg, 4.72 mmol) and 0.5 mL of acetic acid were successively added. The mixture was stirred at room temperature for 60 min, and then sodium triacetoxyborohydride (1.34 g, 6.32 mmol) was added. The resulting mixture was reacted at room temperature for 16 h. To the reaction system was slowly added 80 mL of saturated sodium bicarbonate solution, and the mixture was extracted with 200 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 19a (1.2 g, two-step yield from compound 11a: 64%).

LCMS m/z=487.2 [M+1]⁺

### Step 2: tert-butyl 3-((4-(piperazin-1-ylmethyl)piperidin-1-yl)methyl)azetidine-1-carboxylate (19b)

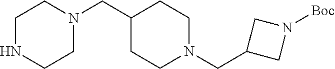 [SMILES: CC(C)(C)OC(=O)N1CC(CN2CCC(CN3CCNCC3)CC2)C1]

19a (1.2 g, 2.47 mmol) was dissolved in 20 mL of methanol, and 0.2 g of 10% palladium on carbon was added. The mixture was stirred at room temperature under hydrogen atmosphere for 16 h. The reaction liquid was filtered, and the filtrate was concentrated under reduced pressure, to afford crude 19b (860 mg).

LCMS m/z=353.2 [M+1]⁺

### Step 3: tert-butyl 3-((4-((4-(4-((4-(2-(3-chloro-4-(2-chloroethoxy)-5-cyanophenyl)propan-2-yl)phenoxy)methyl)pyrimidin-2-yl)piperazin-1-yl)methyl)piperidin-1-yl)methyl) azetidine-1-carboxylate (19c)

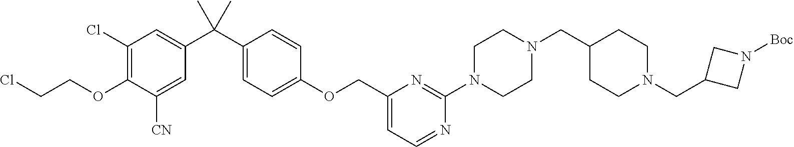 [SMILES: CC(C)(C)OC(=O)N1CC(CN2CCC(CN3CCN(c4nccc(COc5ccc(C(C)(C)c6cc(Cl)c(OCCCl)c(C#N)c6)cc5)n4)CC3)CC2)C1]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (382 mg, 0.80 mmol) was dissolved in mL of DMF, and the above crude tert-butyl 3-((4-(piperazin-1-ylmethyl)piperidin-1-yl)methyl)azetidine-1-carboxylate (19b) (320 mg) and potassium bicarbonate (200 mg, 2.00 mmol) were added. The mixture was warmed to 60° C. and reacted for 3 h. The mixture was cooled to room temperature, and to the reaction system was added 50 mL of water. The aqueous phase was extracted with ethyl acetate (50 mL×3), and the organic phase was washed with water (50 mL×2), dried over anhydrous sodium sulfate, and concentrated under reduced pressure. The crude product was separated and purified by silica gel column chromatography (dichloromethane/methanol (v/v)=15:1), to afford 19c (270 mg, yield from compound 4a: 43%).

LCMS m/z=792.3 [M+1]⁺

### Step 4: 5-(2-(4-((2-(4-((1-(azetidin-3-ylmethyl)piperidin-4-yl)methyl)piperazin-1-yl)pyrimidin-4-yl)methoxy)phenyl)propan-2-yl)-3-chloro-2-(2-chloroethoxy)benzonitrile (19d) trifluoroacetate

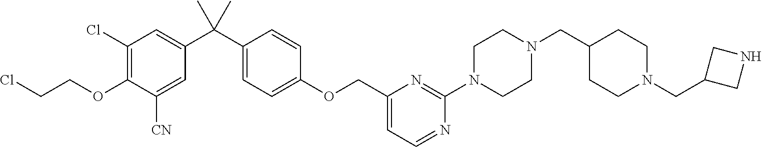 [SMILES: CC(C)(c1ccc(OCc2ccnc(N3CCN(CC4CCN(CC5CNC5)CC4)CC3)n2)cc1)c1cc(Cl)c(OCCCl)c(C#N)c1]

19c (60 mg, 0.076 mmol) was dissolved in 5 mL of DCM, and 2 mL of trifluoroacetic acid was added. The mixture was stirred at room temperature for 2 h. The reaction liquid was concentrated under reduced pressure, to afford crude 19d trifluoroacetate (75 mg).

LC-MS m/z=692.3 [M+1]⁺

### Step 5: 3-chloro-2-(2-chloroethoxy)-5-(2-(4-((2-(4-((1-((1-(2-(2,6-dioxopiperidin-3-yl)-1,3-dioxoisoindolin-5-yl)azetidin-3-yl)methyl)piperidin-4-yl)methyl)piperazin-1-yl)pyrimidin-4-yl)methoxy)phenyl)propan-2-yl)benzonitrile (Compound 19) trifluoroacetate

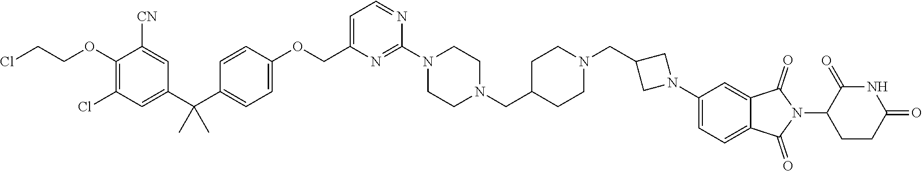 [SMILES: CC(C)(c1ccc(OCc2ccnc(N3CCN(CC4CCN(CC5CN(c6ccc7c(c6)C(=O)N(C6CCC(=O)NC6=O)C7=O)C5)CC4)CC3)n2)cc1)c1cc(Cl)c(OCCCl)c(C#N)c1]

The above crude 19d trifluoroacetate (75 mg) was dissolved in 4 mL of DMSO, and solid sodium bicarbonate (32 mg, 0.38 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (42 mg, 0.15 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 19 trifluoroacetate (10 mg).

¹H NMR (400 MHz, CDCl₃) δ 9.25 (br.s, 1H), 8.34 (d, 1H), 7.55 (d, 1H), 7.45 (d, 1H), 7.28 (d, 1H), 7.09 (d, 2H), 6.94-6.79 (m, 3H), 6.70-6.58 (m, 1H), 6.48-6.34 (m, 1H), 5.04-4.85 (m, 3H), 4.40 (t, 2H), 4.23-4.07 (m, 2H), 3.86 (t, 2H), 3.78-3.63 (m, 2H), 3.62-3.47 (m, 2H), 3.45-3.13 (m, 7H), 3.13-2.94 (m, 4H), 2.94-2.78 (m, 4H), 2.78-2.60 (m, 3H), 2.51-2.36 (m, 1H), 2.24-2.00 (m, 3H), 2.00-1.75 (m, 2H), 1.63 (s, 6H).

LCMS m/z=948.3 [M+1]⁺.

### Example 20

### 3-chloro-2-(2-chloroethoxy)-5-(2-(4-((2-(2-(1-(1-(2-(2,6-dioxopiperidin-3-yl)-1,3-dioxoisoindolin-5-yl)azetidin-3-yl)piperidin-4-yl)-2,7-diazaspiro[3.5]nonan-7-yl)pyrimidin-4-yl)methoxy)phenyl)propan-2-yl)benzonitrile (Compound 20)

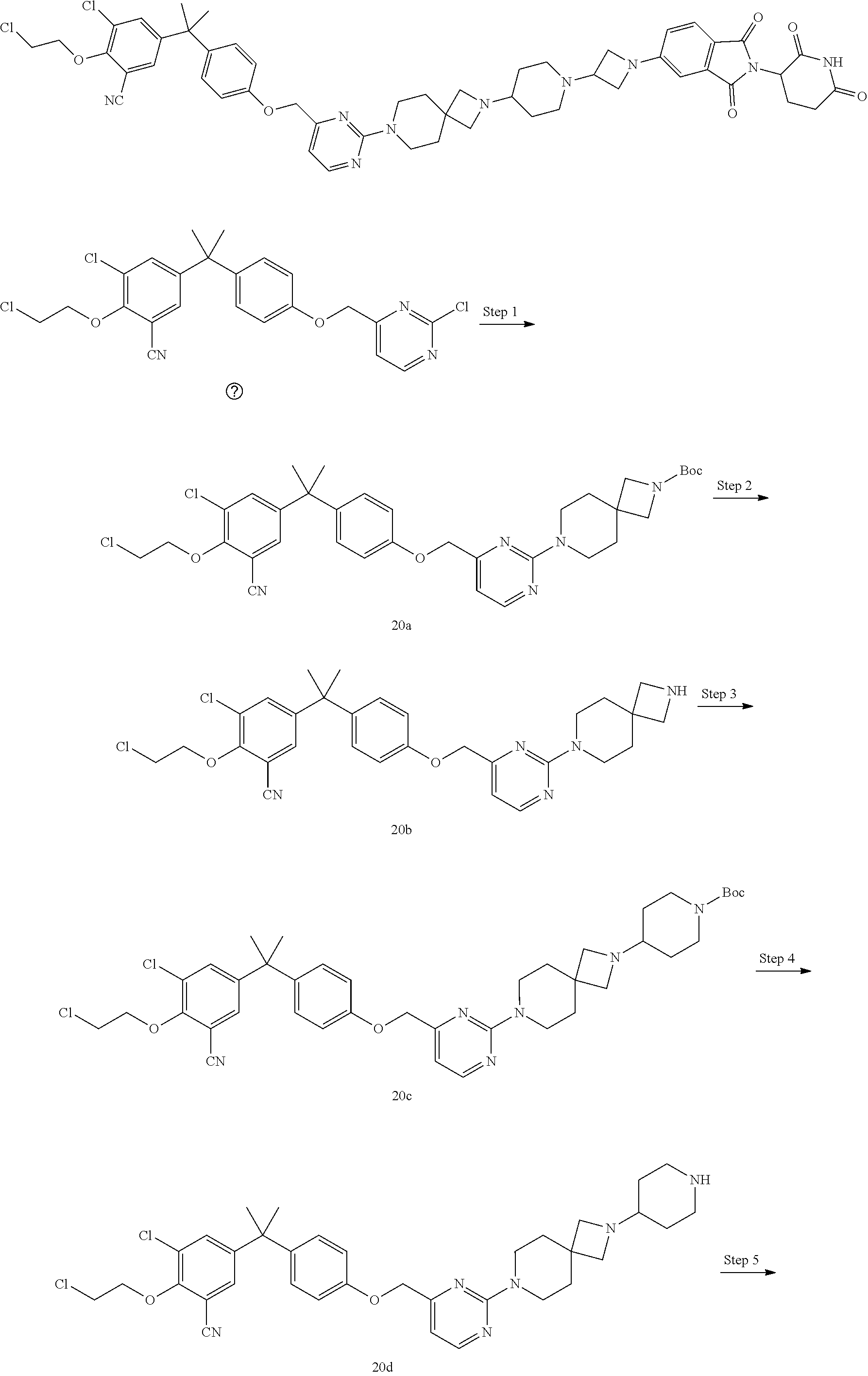

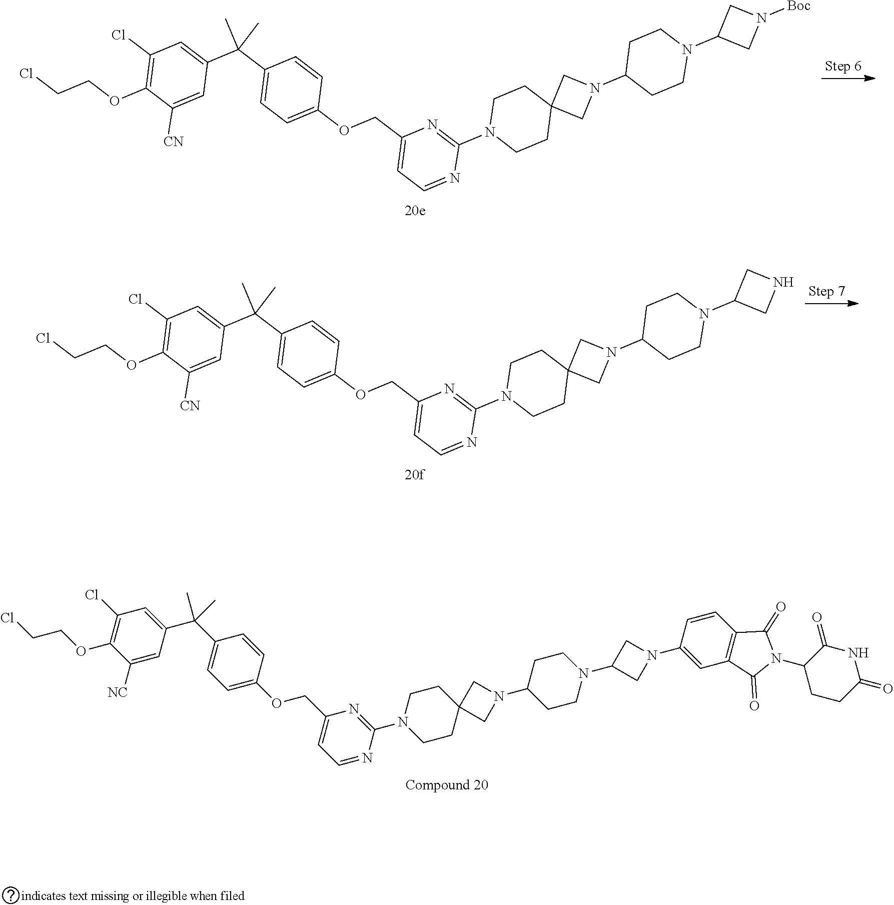 [SMILES: CC(C)(C)OC(=O)N1CC(N2CCC(N3CC4(CCN(c5nccc(COc6ccc(C(C)(C)c7cc(Cl)c(OCCCl)c(C#N)c7)cc6)n5)CC4)C3)CC2)C1 ; CC(C)(c1ccc(OCc2ccnc(N3CCC4(CC3)CN(C3CCN(C5CNC5)CC3)C4)n2)cc1)c1cc(Cl)c(OCCCl)c(C#N)c1 ; CC(C)(c1ccc(OCc2ccnc(N3CCC4(CC3)CN(C3CCN(C5CN(c6ccc7c(c6)C(=O)N(C6CCC(=O)NC6=O)C7=O)C5)CC3)C4)n2)cc1)c1cc(Cl)c(OCCCl)c(C#N)c1]

### Step 1: tert-butyl 7-(4-((4-(2-(3-chloro-4-(2-chloroethoxy)-5-cyanophenyl)propan-2-yl)phenoxy)methyl)pyrimidin-2-yl)-2,7-diazaspiro[3.5]nonane-2-carboxylate (20a)

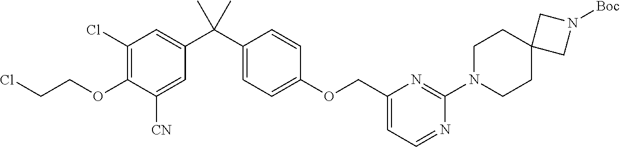 [SMILES: CC(C)(C)OC(=O)N1CC2(CCN(c3nccc(COc4ccc(C(C)(C)c5cc(Cl)c(OCCCl)c(C#N)c5)cc4)n3)CC2)C1]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (300 mg, 0.63 mmol) was dissolved in 20 mL of DMF, and tert-butyl 2,7-diazaspiro[3.5]nonane-2-carboxylate (171 mg, 0.76 mmol) and potassium bicarbonate (126 mg, 1.26 mmol) were successively added. The mixture was warmed to 60° C. and reacted for 3 h. The mixture was cooled to room temperature, and to the reaction system was added 50 mL of water. The mixture was extracted with ethyl acetate (40 mL×3), and the organic phase was washed with water (50 mL×2), dried over anhydrous sodium sulfate, and concentrated under reduced pressure. The crude product was separated and purified by silica gel column chromatography (petroleum ether/ethyl acetate (v/v)=2:1), to afford 20a (370 mg, yield: 88%).

LCMS m/z=666.2 [M+1]⁺

### Step 2: 5-(2-(4-((2-(2,7-diazaspiro[3.5]nonan-7-yl)pyrimidin-4-yl)methoxy)phenyl)propan-2-yl)-3-chloro-2-(2-chloroethoxy)benzonitrile (20b) trifluoroacetate

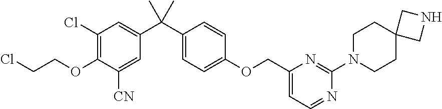 [SMILES: CC(C)(c1ccc(OCc2ccnc(N3CCC4(CC3)CNC4)n2)cc1)c1cc(Cl)c(OCCCl)c(C#N)c1]

20a (0.37 g, 0.56 mmol) was dissolved in 10 mL of DCM, and 4 mL of trifluoroacetic acid was added. The mixture was stirred at room temperature for 2 h. The reaction liquid was concentrated under reduced pressure, to afford crude 20b trifluoroacetate (0.44 g).

LCMS m/z=566.2 [M+1]⁺

### Step 3: tert-butyl 4-(7-(4-((4-(2-(3-chloro-4-(2-chloroethoxy)-5-cyanophenyl)propan-2-yl)phenoxy)methyl)pyrimidin-2-yl)-2,7-diazaspiro[3.5]nonan-2-yl)piperidine-1-carboxylate (20c)

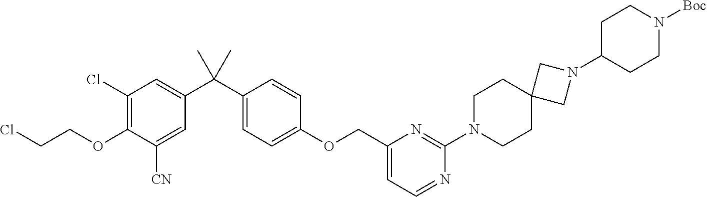 [SMILES: CC(C)(C)OC(=O)N1CCC(N2CC3(CCN(c4nccc(COc5ccc(C(C)(C)c6cc(Cl)c(OCCCl)c(C#N)c6)cc5)n4)CC3)C2)CC1]

The above crude 20b trifluoroacetate (440 mg) was dissolved in 15 mL of DCE, and solid sodium bicarbonate (132 mg, 1.57 mmol) was added. The mixture was stirred at room temperature for 20 min, and then N-Boc-4-piperidone (210 mg, 1.06 mmol) and 0.1 mL of acetic acid were successively added. The mixture was stirred at room temperature for 60 min, and then sodium triacetoxyborohydride (220 mg, 1.04 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 20c (400 mg, two-step yield from compound 20a: 95%).

LCMS m/z=749.3 [M+1]⁺

### Step 4: 3-chloro-2-(2-chloroethoxy)-5-(2-(4-((2-(2-(piperidin-4-yl)-2,7-diazaspiro[3.5]nonan-7-yl)pyrimidin-4-yl)methoxy)phenyl)propan-2-yl)benzonitrile (20d) trifluoroacetate

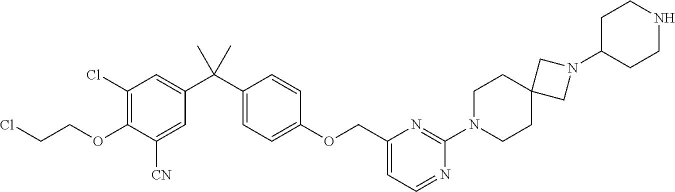 [SMILES: CC(C)(c1ccc(OCc2ccnc(N3CCC4(CC3)CN(C3CCNCC3)C4)n2)cc1)c1cc(Cl)c(OCCCl)c(C#N)c1]

20c (200 mg, 0.27 mmol) was dissolved in 10 mL of DCM, and 4 mL of trifluoroacetic acid was added. The mixture was stirred at room temperature for 2 h. The reaction liquid was concentrated under reduced pressure, to afford crude 20d trifluoroacetate (240 mg).

LCMS m/z=649.3 [M+1]⁺

### Step 5: tert-butyl 3-(4-(7-(4-((4-(2-(3-chloro-4-(2-chloroethoxy)-5-cyanophenyl)propan-2-yl)phenoxy)methyl)pyrimidin-2-yl)-2,7-diazaspiro[3.5]nonan-2-yl)piperidin-1-yl)azetidine-1-carboxylate (20e)

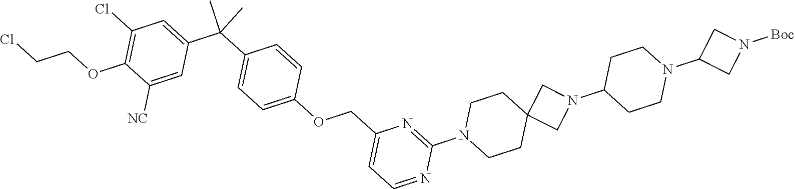 [SMILES: CC(C)(C)OC(=O)N1CC(N2CCC(N3CC4(CCN(c5nccc(COc6ccc(C(C)(C)c7cc(Cl)c(OCCCl)c(C#N)c7)cc6)n5)CC4)C3)CC2)C1]

The above crude 20d trifluoroacetate (240 mg) was dissolved in 15 mL of DCE, and solid sodium bicarbonate (112 mg, 1.33 mmol) was added. The mixture was stirred at room temperature for 20 min, and then 1-Boc-3-azetidinone (91 mg, 0.53 mmol) and 0.05 mL of acetic acid were successively added. The mixture was stirred at room temperature for 60 min, and then sodium triacetoxyborohydride (115 mg, 0.54 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 20e (200 mg, two-step yield from compound 20c: 92%).

LCMS m/z=804.3 [M+1]⁺

### Step 6: 5-(2-(4-((2-(2-(1-(azetidin-3-yl)piperidin-4-yl)-2,7-diazaspiro[3.5]nonan-7-yl)pyrimidin-4-yl)methoxy)phenyl)propan-2-yl)-3-chloro-2-(2-chloroethoxy)benzonitrile (20f) trifluoroacetate

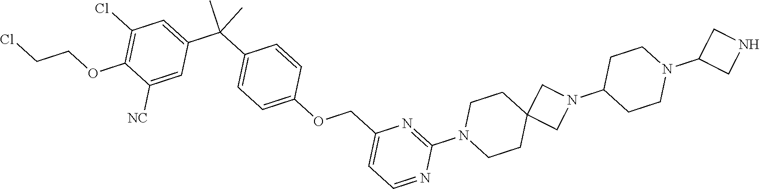 [SMILES: CC(C)(c1ccc(OCc2ccnc(N3CCC4(CC3)CN(C3CCN(C5CNC5)CC3)C4)n2)cc1)c1cc(Cl)c(OCCCl)c(C#N)c1]

20e (50 mg, 0.062 mmol) was dissolved in 5 mL of DCM, and 2 mL of trifluoroacetic acid was added. The mixture was stirred at room temperature for 2 h. The reaction liquid was concentrated under reduced pressure, to afford crude 20f trifluoroacetate (60 mg).

LCMS m/z=704.3 [M+1]⁺

### Step 7: 3-chloro-2-(2-chloroethoxy)-5-(2-(4-((2-(2-(1-(1-(2-(2,6-dioxopiperidin-3-yl)-1,3-dioxoisoindolin-5-yl)azetidin-3-yl)piperidin-4-yl)-2,7-diazaspiro[3.5]nonan-7-yl)pyrimidin-4-yl)methoxy)phenyl)propan-2-yl)benzonitrile (Compound 20)

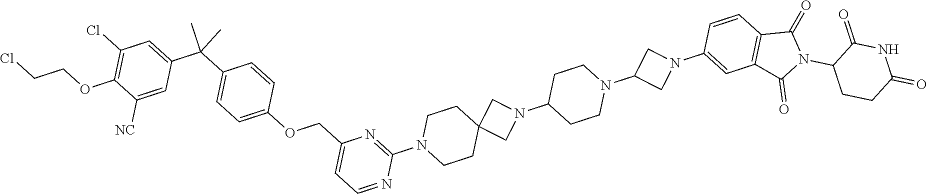 [SMILES: CC(C)(c1ccc(OCc2ccnc(N3CCC4(CC3)CN(C3CCN(C5CN(c6ccc7c(c6)C(=O)N(C6CCC(=O)NC6=O)C7=O)C5)CC3)C4)n2)cc1)c1cc(Cl)c(OCCCl)c(C#N)c1]

The above crude 20f trifluoroacetate (60 mg) was dissolved in 4 mL of DMSO, and solid sodium bicarbonate (25 mg, 0.30 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 20 (15 mg, two-step yield from compound 20e: 25%).

¹H NMR (400 MHz, CDCl₃) δ 8.29 (d, 1H), 8.04 (br.s, 1H), 7.64 (d, 1H), 7.44 (d, 1H), 7.31 (d, 1H), 7.13-7.04 (m, 2H), 6.92-6.84 (m, 2H), 6.78 (d, 1H), 6.68 (d, 1H), 6.52 (dd, 1H), 5.00-4.85 (m, 3H), 4.41 (t, 2H), 4.15-4.02 (m, 2H), 3.94-3.82 (m, 4H), 3.82-3.70 (m, 4H), 3.43-3.33 (m, 1H), 3.33-2.94 (m, 4H), 2.94-2.65 (m, 5H), 2.29-2.07 (m, 2H), 2.07-1.95 (m, 2H), 1.93-1.70 (m, 6H), 1.70-1.52 (m, 8H).

LCMS m/z=960.2 [M+1]⁺.

### Example 21

### 3-chloro-2-(2-chloroethoxy)-5-(2-(4-((2-(2-(1-((1-(2-(2,6-dioxopiperidin-3-yl)-1,3-dioxoisoindolin-5-yl)piperidin-4-yl)methyl)piperidin-4-yl)-2,7-diazaspiro[3.5]nonan-7-yl)pyrimidin-4-yl)methoxy)phenyl)propan-2-yl)benzonitrile (Compound 21)

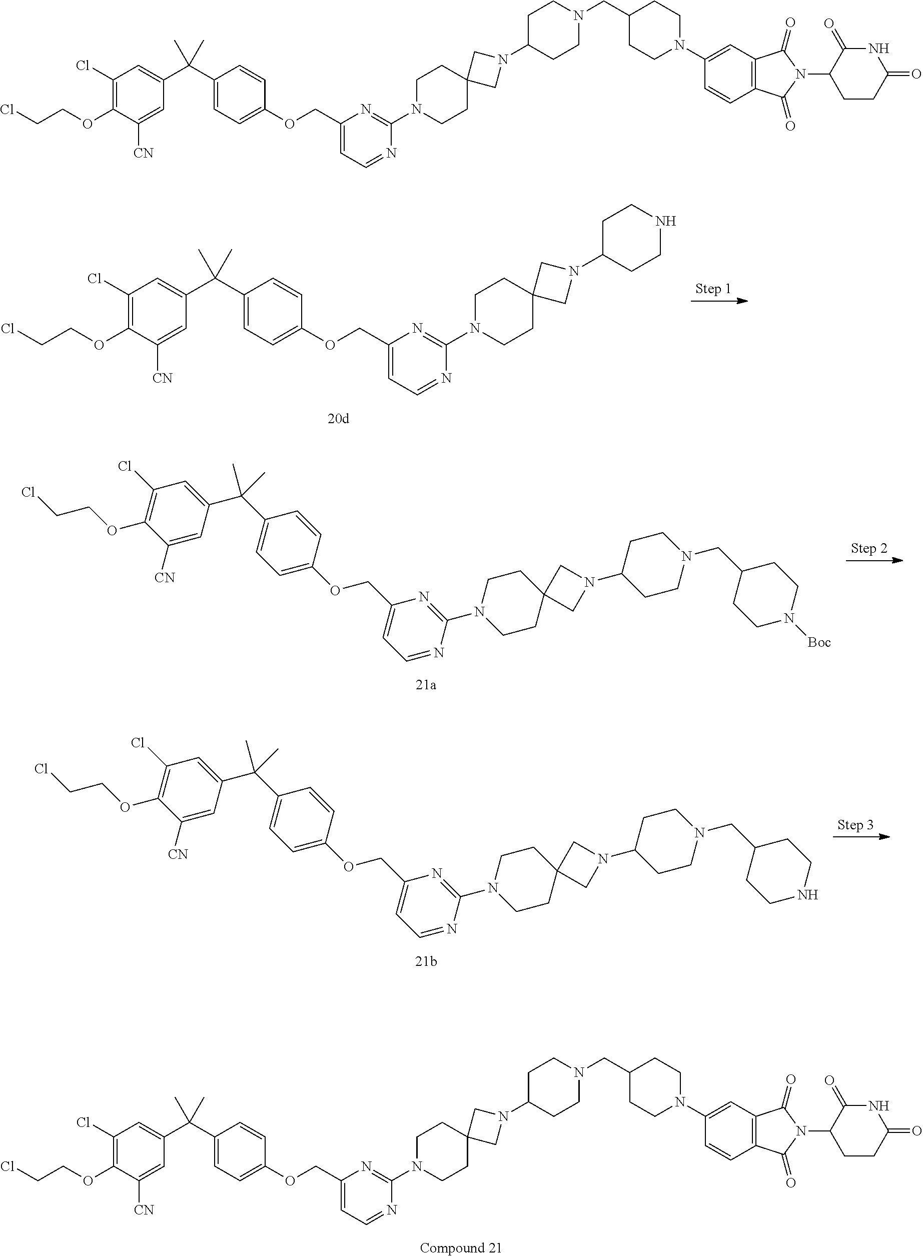

### Step 1: tert-butyl 4-((4-(7-(4-((4-(2-(3-chloro-4-(2-chloroethoxy)-5-cyanophenyl)propan-2-yl)phenoxy)methyl)pyrimidin-2-yl)-2,7-diazaspiro[3.5]nonan-2-yl)piperidin-1-yl)methyl) piperidine-1-carboxylate (21a)

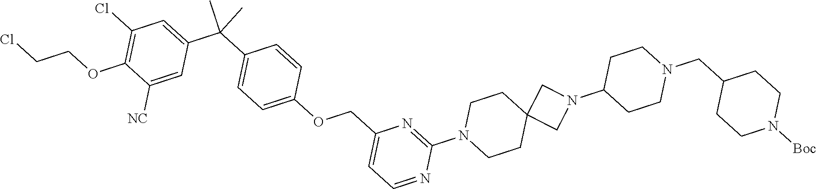 [SMILES: CC(C)(C)OC(=O)N1CCC(CN2CCC(N3CC4(CCN(c5nccc(COc6ccc(C(C)(C)c7cc(Cl)c(OCCCl)c(C#N)c7)cc6)n5)CC4)C3)CC2)CC1]

The above crude 20d trifluoroacetate (240 mg) was dissolved in 15 mL of DCE, and solid sodium bicarbonate (112 mg, 1.33 mmol) was added. The mixture was stirred at room temperature for 20 min, and then tert-butyl 4-formylpiperidine-1-carboxylate (114 mg, 0.53 mmol) and 0.05 mL of acetic acid were successively added. The mixture was stirred at room temperature for 60 min, and then sodium triacetoxyborohydride (115 mg, 0.54 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 21a (210 mg, two-step yield from compound 20c: 92%).

LCMS m/z=846.4 [M+1]⁺

### Step 2: 3-chloro-2-(2-chloroethoxy)-5-(2-(4-((2-(2-(1-(piperidin-4-ylmethyl)piperidin-4-yl)-2,7-diazaspiro[3.5]nonan-7-yl)pyrimidin-4-yl)methoxy)phenyl)propan-2-yl)benzonitrile (21b) trifluoroacetate

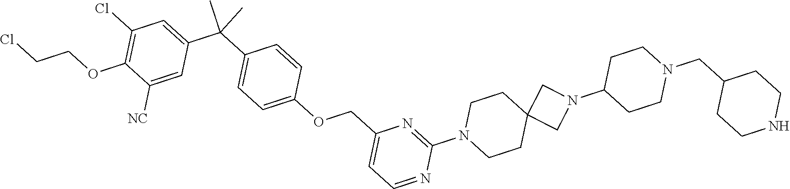 [SMILES: CC(C)(c1ccc(OCc2ccnc(N3CCC4(CC3)CN(C3CCN(CC5CCNCC5)CC3)C4)n2)cc1)c1cc(Cl)c(OCCCl)c(C#N)c1]

21a (50 mg, 0.059 mmol) was dissolved in 5 mL of DCM, and 2 mL of trifluoroacetic acid was added. The mixture was stirred at room temperature for 2 h. The reaction liquid was concentrated under reduced pressure, to afford crude 21b trifluoroacetate (60 mg).

LC-MS m/z=746.4 [M+1]⁺

### Step 3: 3-chloro-2-(2-chloroethoxy)-5-(2-(4-((2-(2-(1-((1-(2-(2,6-dioxopiperidin-3-yl)-1,3-dioxoisoindolin-5-yl)piperidin-4-yl)methyl)piperidin-4-yl)-2,7-diazaspiro[3.5]nonan-7-yl)pyrimidin-4-yl)methoxy)phenyl)propan-2-yl)benzonitrile (Compound 21)

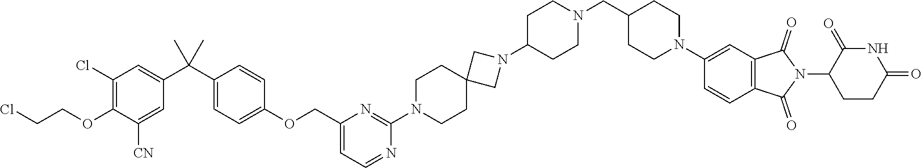 [SMILES: CC(C)(c1ccc(OCc2ccnc(N3CCC4(CC3)CN(C3CCN(CC5CCN(c6ccc7c(c6)C(=O)N(C6CCC(=O)NC6=O)C7=O)CC5)CC3)C4)n2)cc1)c1cc(Cl)c(OCCCl)c(C#N)c1]

The above crude 21b trifluoroacetate (60 mg) was dissolved in 4 mL of DMSO, and solid sodium bicarbonate (25 mg, 0.30 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 21 (20 mg, two-step yield from compound 21a: 34%).

¹H NMR (400 MHz, CDCl₃) δ 8.29 (d, 1H), 8.19 (br.s, 1H), 7.66 (d, 1H), 7.44 (d, 1H), 7.32 (d, 1H), 7.29-7.26 (m, 1H), 7.16-6.98 (m, 3H), 6.94-6.82 (m, 2H), 6.68 (d, 1H), 5.00-4.86 (m, 3H), 4.14 (t, 2H), 4.02-3.82 (m, 4H), 3.82-3.70 (m, 4H), 3.40-2.55 (m, 11H), 2.32-2.08 (m, 4H), 2.07-1.93 (m, 2H), 1.93-1.84 (m, 2H), 1.84-1.77 (m, 4H), 1.77-1.67 (m, 3H), 1.67-1.56 (m, 7H), 1.54-1.34 (m, 1H), 1.34-1.19 (m, 2H).

LCMS m/z=1002.3 [M+1]⁺.

### Example 22

### 3-chloro-2-(2-chloroethoxy)-5-(2-(4-((2-(2-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)-2,7-diazaspiro[3.5]nonan-7-yl)pyrimidin-4-yl)methoxy)phenyl)propan-2-yl)benzonitrile (Compound 22) trifluoroacetate

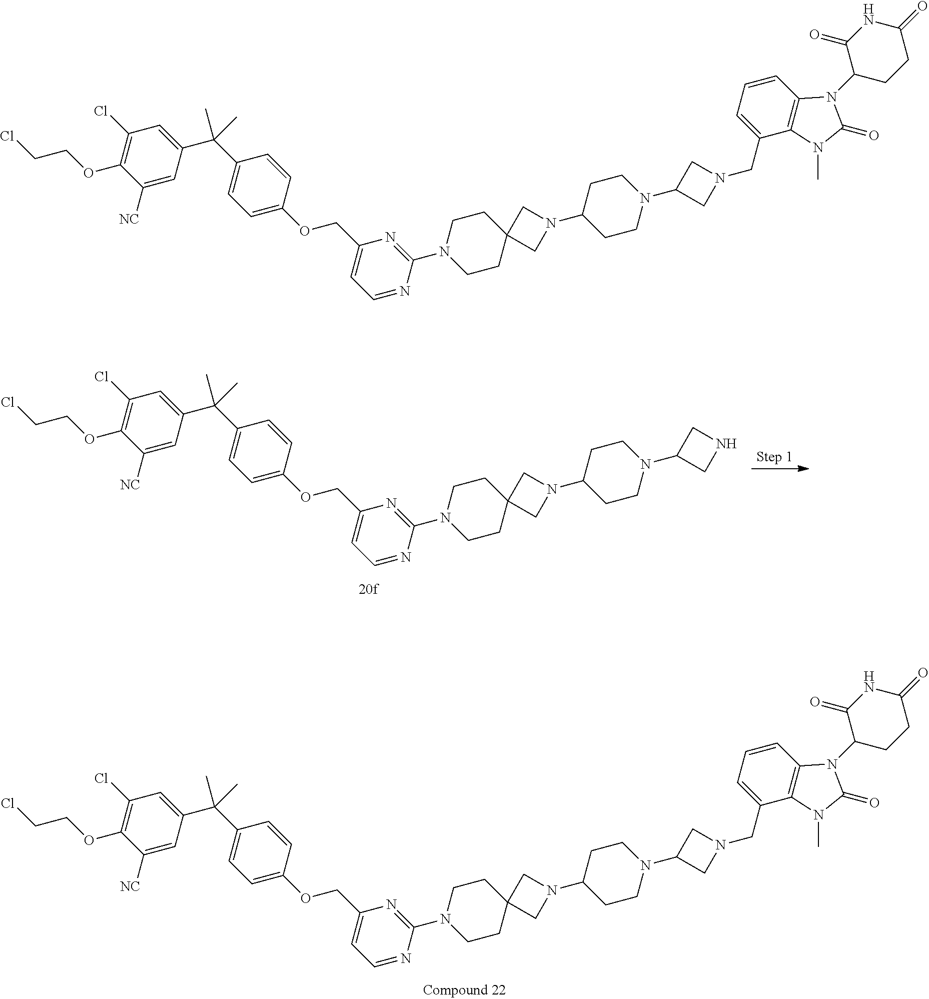 [SMILES: Cn1c(=O)n(C2CCC(=O)NC2=O)c2cccc(CN3CC(N4CCC(N5CC6(CCN(c7nccc(COc8ccc(C(C)(C)c9cc(Cl)c(OCCCl)c(C#N)c9)cc8)n7)CC6)C5)CC4)C3)c21 ; CC(C)(c1ccc(OCc2ccnc(N3CCC4(CC3)CN(C3CCN(C5CNC5)CC3)C4)n2)cc1)c1cc(Cl)c(OCCCl)c(C#N)c1 ; Cn1c(=O)n(C2CCC(=O)NC2=O)c2cccc(CN3CC(N4CCC(N5CC6(CCN(c7nccc(COc8ccc(C(C)(C)c9cc(Cl)c(OCCCl)c(C#N)c9)cc8)n7)CC6)C5)CC4)C3)c21]

The above crude 20f trifluoroacetate (60 mg) was dissolved in 4 mL of anhydrous THF, and solid sodium bicarbonate (25 mg, 0.30 mmol) was added. The mixture was stirred at room temperature for 10 min, and then 1-(2,6-dioxopiperidin-3-yl)-3-methyl-2-oxo-2,3-dihydro-1H-benzo[d]imidazole-4-carbaldehyde (see WO 2020113233 for the synthetic method) (40 mg, 0.14 mmol) and acetic acid (0.02 mL, 0.35 mmol) were successively added. The mixture was stirred at room temperature for 2 h, and then sodium triacetoxyborohydride (26 mg, 0.12 mmol) was added. The resulting mixture was reacted at room temperature for 16 h. To the reaction liquid was added solid sodium bicarbonate (100 mg, 1.19 mmol), and the mixture was stirred at room temperature for 15 min and then filtered. The filtrate was concentrated under reduced pressure, and the resulting crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 22 trifluoroacetate (12 mg).

¹H NMR (400 MHz, DMSO-d₆) δ 11.11 (s, 1H), 8.36 (d, 1H), 7.61 (d, 1H), 7.57 (d, 1H), 7.27-7.05 (m, 5H), 6.97-6.90 (m, 2H), 6.70 (d, 1H), 5.42 (dd, 1H), 4.97 (s, 2H), 4.80-4.60 (m, 2H), 4.45-4.38 (m, 2H), 4.25-4.13 (m, 2H), 4.08-3.90 (m, 8H), 3.85-3.73 (m, 2H), 3.73-3.63 (m, 2H), 3.59 (s, 3H), 3.51-3.35 (m, 1H), 3.35-3.17 (m, 2H), 3.14-2.97 (m, 2H), 2.97-2.80 (m, 1H), 2.79-2.58 (m, 2H), 2.08-1.90 (m, 4H), 1.90-1.80 (m, 2H), 1.80-1.70 (m, 2H), 1.63 (s, 6H), 1.50-1.28 (m, 2H).

LCMS m/z=975.4 [M+1]⁺.

### Example 23

### 3-chloro-2-(2-chloroethoxy)-5-(2-(4-((2-(7-(1-(1-(2-(2,6-dioxopiperidin-3-yl)-1,3-dioxoisoindolin-5-yl)azetidin-3-yl)piperidin-4-yl)-2,7-diazaspiro[3.5]nonan-2-yl)pyrimidin-4-yl)methoxy)phenyl)propan-2-yl)benzonitrile (Compound 23)

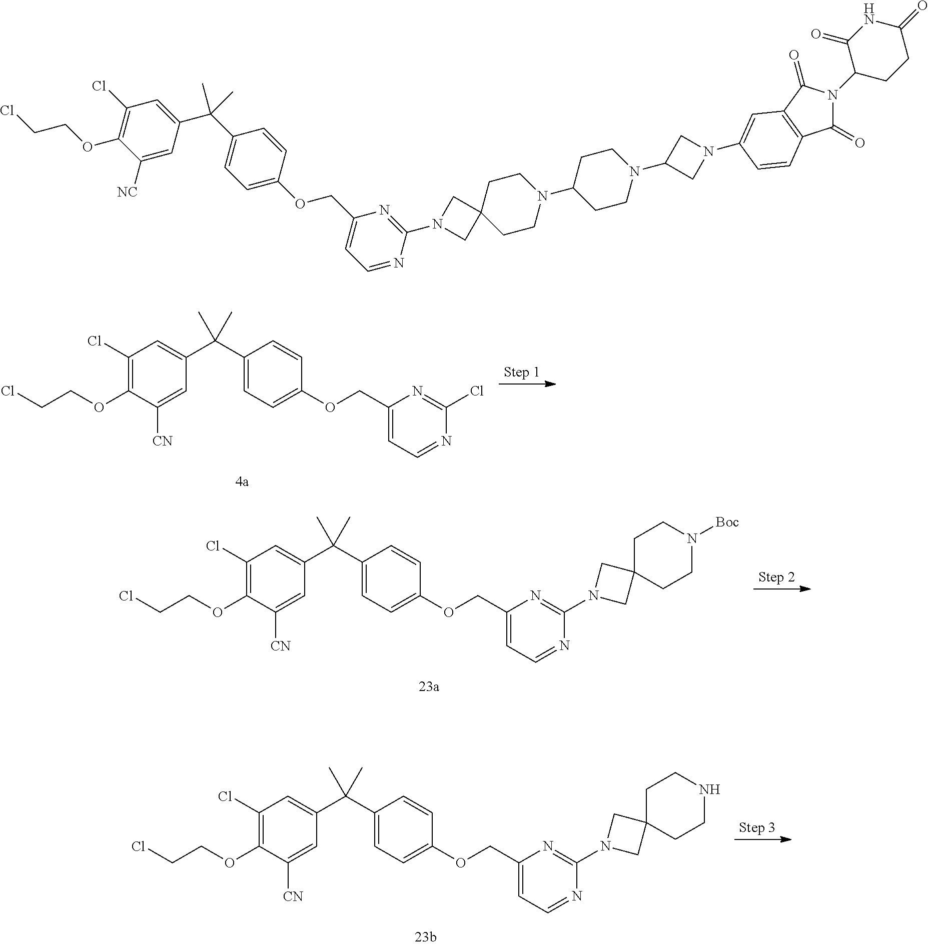 [SMILES: CC(C)(c1ccc(OCc2ccnc(N3CC4(CCN(C5CCN(C6CN(c7ccc8c(c7)C(=O)N(C7CCC(=O)NC7=O)C8=O)C6)CC5)CC4)C3)n2)cc1)c1cc(Cl)c(OCCCl)c(C#N)c1 ; CC(C)(c1ccc(OCc2ccnc(Cl)n2)cc1)c1cc(Cl)c(OCCCl)c(C#N)c1 ; CC(C)(C)OC(=O)N1CCC2(CC1)CN(c1nccc(COc3ccc(C(C)(C)c4cc(Cl)c(OCCCl)c(C#N)c4)cc3)n1)C2 ; CC(C)(c1ccc(OCc2ccnc(N3CC4(CCNCC4)C3)n2)cc1)c1cc(Cl)c(OCCCl)c(C#N)c1]

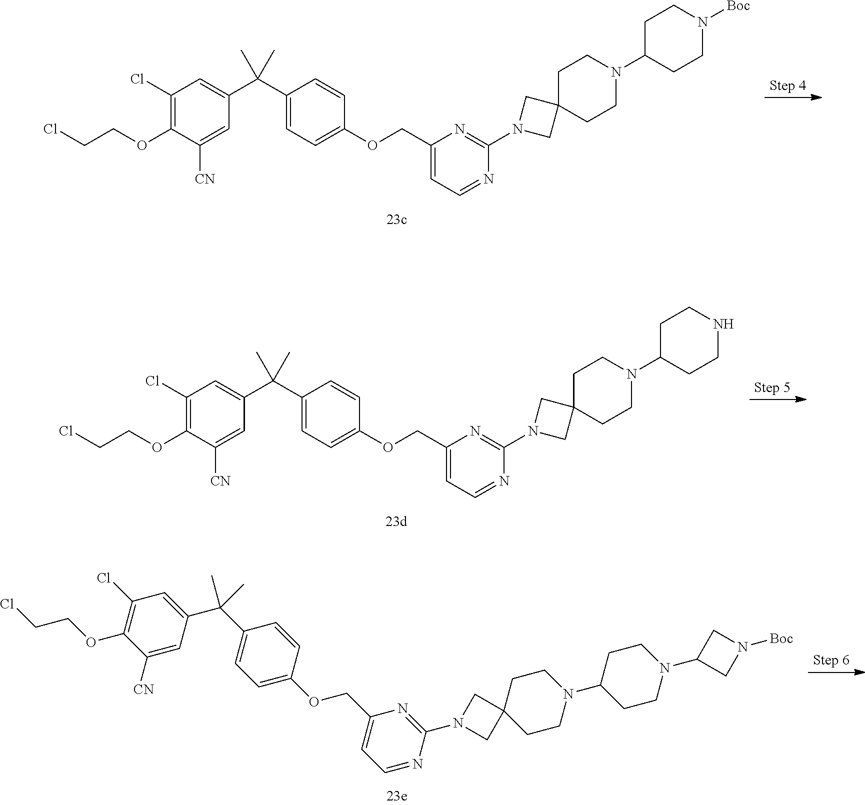 [SMILES: CC(C)(C)OC(=O)N1CCC(N2CCC3(CC2)CN(c2nccc(COc4ccc(C(C)(C)c5cc(Cl)c(OCCCl)c(C#N)c5)cc4)n2)C3)CC1 ; CC(C)(c1ccc(OCc2ccnc(N3CC4(CCN(C5CCNCC5)CC4)C3)n2)cc1)c1cc(Cl)c(OCCCl)c(C#N)c1 ; CC(C)(C)OC(=O)N1CC(N2CCC(N3CCC4(CC3)CN(c3nccc(COc5ccc(C(C)(C)c6cc(Cl)c(OCCCl)c(C#N)c6)cc5)n3)C4)CC2)C1]

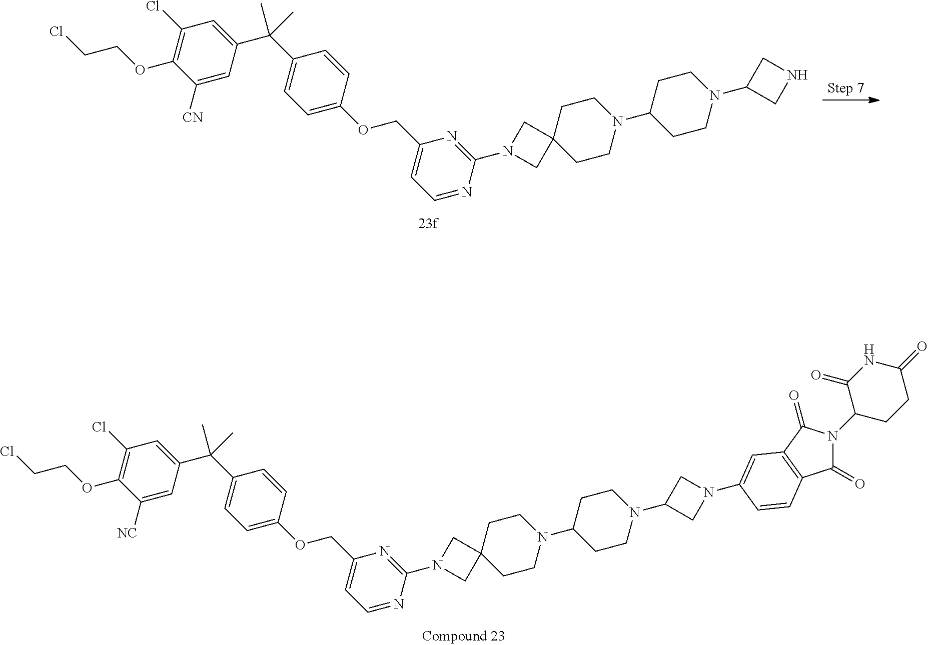 [SMILES: CC(C)(c1ccc(OCc2ccnc(N3CC4(CCN(C5CCN(C6CNC6)CC5)CC4)C3)n2)cc1)c1cc(Cl)c(OCCCl)c(C#N)c1 ; CC(C)(c1ccc(OCc2ccnc(N3CC4(CCN(C5CCN(C6CN(c7ccc8c(c7)C(=O)N(C7CCC(=O)NC7=O)C8=O)C6)CC5)CC4)C3)n2)cc1)c1cc(Cl)c(OCCCl)c(C#N)c1]

### Step 1: tert-butyl 2-(4-((4-(2-(3-chloro-4(2-chloroethoxy)-5-cyanophenyl)propan-2-yl)phenoxy)methyl)pyrimidin-2-yl)-2,7-diazaspiro[3.5]nonane-7-carboxylate (23a)

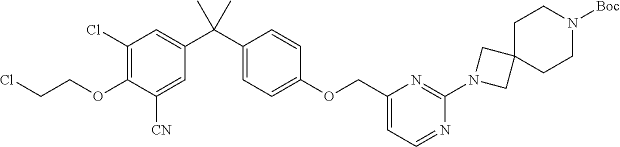 [SMILES: CC(C)(C)OC(=O)N1CCC2(CC1)CN(c1nccc(COc3ccc(C(C)(C)c4cc(Cl)c(OCCCl)c(C#N)c4)cc3)n1)C2]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (300 mg, 0.63 mmol) was dissolved in 20 mL of DMF, and tert-butyl 2,7-diazaspiro[3.5]nonane-7-carboxylate (171 mg, 0.76 mmol) and potassium bicarbonate (126 mg, 1.26 mmol) were successively added. The mixture was warmed to 60° C. and reacted for 3 h. The mixture was cooled to room temperature, and to the reaction system was added 50 mL of water. The mixture was extracted with ethyl acetate (40 mL×3), and the organic phase was washed with water (50 mL×2), dried over anhydrous sodium sulfate, and concentrated under reduced pressure. The crude product was separated and purified by silica gel column chromatography (petroleum ether/ethyl acetate (v/v)=2:1), to afford 23a (400 mg, yield: 95%).

LCMS m/z=666.2 [M+1]⁺

### Step 2: 5-(2-(4-((2-(2,7-diazaspiro[3.5]nonan-2-yl)pyrimidin-4-yl)methoxy)phenyl)propan-2-yl)-3-chloro-2-(2-chloroethoxy)benzonitrile (23b) trifluoroacetate

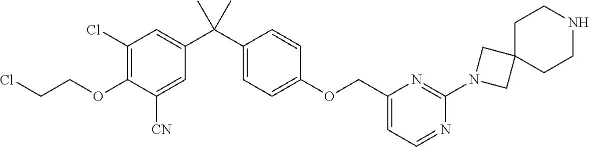 [SMILES: CC(C)(c1ccc(OCc2ccnc(N3CC4(CCNCC4)C3)n2)cc1)c1cc(Cl)c(OCCCl)c(C#N)c1]

23a (0.30 g, 0.45 mmol) was dissolved in 10 mL of DCM, and 4 mL of trifluoroacetic acid was added. The mixture was stirred at room temperature for 2 h. The reaction liquid was concentrated under reduced pressure, to afford crude 23b trifluoroacetate (0.36 g).

LCMS m/z=566.2 [M+1]⁺

### Step 3: tert-butyl 4-(2-(4-((4-(2-(3-chloro-4-(2-chloroethoxy)-5-cyanophenyl)propan-2-yl)phenoxy)methyl)pyrimidin-2-yl)-2,7-diazaspiro[3.5]nonan-7-yl)piperidine-1-carboxylate (23c)

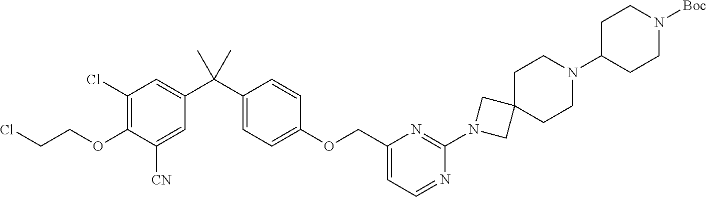 [SMILES: CC(C)(C)OC(=O)N1CCC(N2CCC3(CC2)CN(c2nccc(COc4ccc(C(C)(C)c5cc(Cl)c(OCCCl)c(C#N)c5)cc4)n2)C3)CC1]

The above crude 23b trifluoroacetate (0.36 g) was dissolved in 15 mL of DCE, and solid sodium bicarbonate (115 mg, 1.37 mmol) was added. The mixture was stirred at room temperature for 20 min, and then N-Boc-4-piperidone (180 mg, 0.90 mmol) and 0.1 mL of acetic acid were added. The mixture was stirred at room temperature for 60 min, and then sodium triacetoxyborohydride (191 mg, 0.90 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 23c (320 mg, two-step yield from compound 23a: 95%).

LCMS m/z=749.3 [M+1]⁺

### Step 4: 3-chloro-2-(2-chloroethoxy)-5-(2-(4-((2-(7-(piperidin-4-yl)-2,7-diazaspiro[3.5]nonan-2-yl)pyrimidin-4-yl)methoxy)phenyl)propan-2-yl)benzonitrile (23d) trifluoroacetate

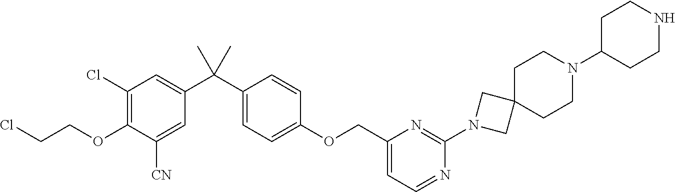 [SMILES: CC(C)(c1ccc(OCc2ccnc(N3CC4(CCN(C5CCNCC5)CC4)C3)n2)cc1)c1cc(Cl)c(OCCCl)c(C#N)c1]

23c (160 mg, 0.21 mmol) was dissolved in 10 mL of DCM, and 4 mL of trifluoroacetic acid was added. The mixture was stirred at room temperature for 2 h. The reaction liquid was concentrated under reduced pressure, to afford crude 23d trifluoroacetate (200 mg).

LCMS m/z=649.3 [M+1]⁺

### Step 5: tert-butyl 3-(4-(2-(4-((4-(2-(3-chloro-4-(2-chloroethoxy)-5-cyanophenyl)propan-2-yl)phenoxy)methyl)pyrimidin-2-yl)-2,7-diazaspiro[3.5]nonan-7-yl)piperidin-1-yl)azetidine-1-carboxylate (23e)

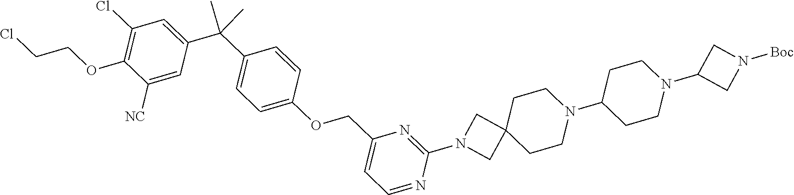 [SMILES: CC(C)(C)OC(=O)N1CC(N2CCC(N3CCC4(CC3)CN(c3nccc(COc5ccc(C(C)(C)c6cc(Cl)c(OCCCl)c(C#N)c6)cc5)n3)C4)CC2)C1]

The crude 23d trifluoroacetate (200 mg) was dissolved in 15 mL of DCE, and solid sodium bicarbonate (74 mg, 0.88 mmol) was added. The mixture was stirred at room temperature for 20 min, and then 1-Boc-3-azetidinone (75 mg, 0.44 mmol) and 0.05 mL of acetic acid were successively added. The mixture was stirred at room temperature for 60 min, and then sodium triacetoxyborohydride (94 mg, 0.44 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 23e (140 mg, two-step yield from compound 23c: 83%).

LCMS m/z=804.3 [M+1]⁺

### Step 6: 5-(2-(4-((2-(7-(1-(azetidin-3-yl)piperidin-4-yl)-2,7-diazaspiro[3.5]nonan-2-yl)pyrimidin-4-yl)methoxy)phenyl)propan-2-yl)-3-chloro-2-(2-chloroethoxy)benzonitrile (23f) trifluoroacetate

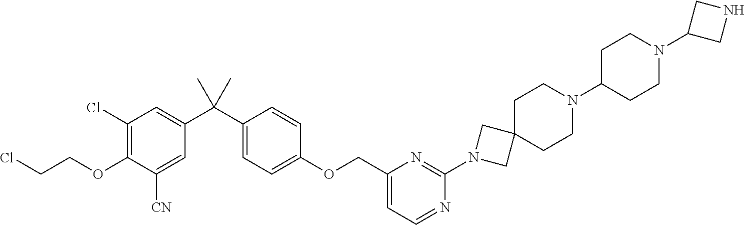 [SMILES: CC(C)(c1ccc(OCc2ccnc(N3CC4(CCN(C5CCN(C6CNC6)CC5)CC4)C3)n2)cc1)c1cc(Cl)c(OCCCl)c(C#N)c1]

23e (50 mg, 0.062 mmol) was dissolved in 5 mL of DCM, and 2 mL of trifluoroacetic acid was added. The mixture was stirred at room temperature for 2 h. The reaction liquid was concentrated under reduced pressure, to afford crude 23f trifluoroacetate (60 mg).

LC-MS m/z=704.3 [M+1]⁺

### Step 7: 3-chloro-2-(2-chloroethoxy)-5-(2-(4-((2-(7-(1-(1-(2-(2,6-dioxopiperidin-3-yl)-1,3-dioxoisoindolin-5-yl)azetidin-3-yl)piperidin-4-yl)-2,7-diazaspiro[3.5]nonan-2-yl)pyrimidin-4-yl)methoxy)phenyl)propan-2-yl)benzonitrile (Compound 23)

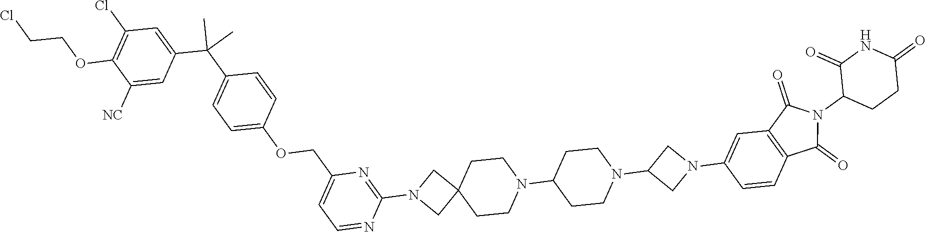 [SMILES: CC(C)(c1ccc(OCc2ccnc(N3CC4(CCN(C5CCN(C6CN(c7ccc8c(c7)C(=O)N(C7CCC(=O)NC7=O)C8=O)C6)CC5)CC4)C3)n2)cc1)c1cc(Cl)c(OCCCl)c(C#N)c1]

The above crude 23f trifluoroacetate (60 mg) was dissolved in 4 mL of DMSO, and solid sodium bicarbonate (25 mg, 0.30 mmol) was added. The mixture was stirred at room temperature for 10 min, and 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 23 (20 mg, two-step yield from compound 23e: 34%).

¹H NMR (400 MHz, CDCl₃) δ 8.31 (d, 1H), 8.04 (br.s, 1H), 7.64 (d, 1H), 7.44 (d, 1H), 7.31 (d, 1H), 7.15-7.05 (m, 2H), 6.94-6.73 (m, 4H), 6.52 (dd, 1H), 5.00-4.87 (m, 3H), 4.41 (t, 2H), 4.18-4.03 (m, 2H), 3.95-3.79 (m, 8H), 3.42-3.29 (m, 1H), 3.02-2.92 (m, 2H), 2.92-2.66 (m, 4H), 2.66-2.30 (m, 4H), 2.21-2.08 (m, 1H), 2.02-1.78 (m, 6H), 1.75-1.51 (m, 10H).

LCMS m/z=960.2 [M+1]⁺.

### Example 24

### 3-chloro-2-(2-chloroethoxy)-5-(2-(4-((2-(7-(1-((1-(2-(2,6-dioxopiperidin-3-yl)-1,3-dioxoisoindolin-5-yl)piperidin-4-yl)methyl)piperidin-4-yl)-2,7-diazaspiro[3.5]nonan-2-yl)pyrimidin-4-yl)methoxy)phenyl)propan-2-yl)benzonitrile (Compound 24)

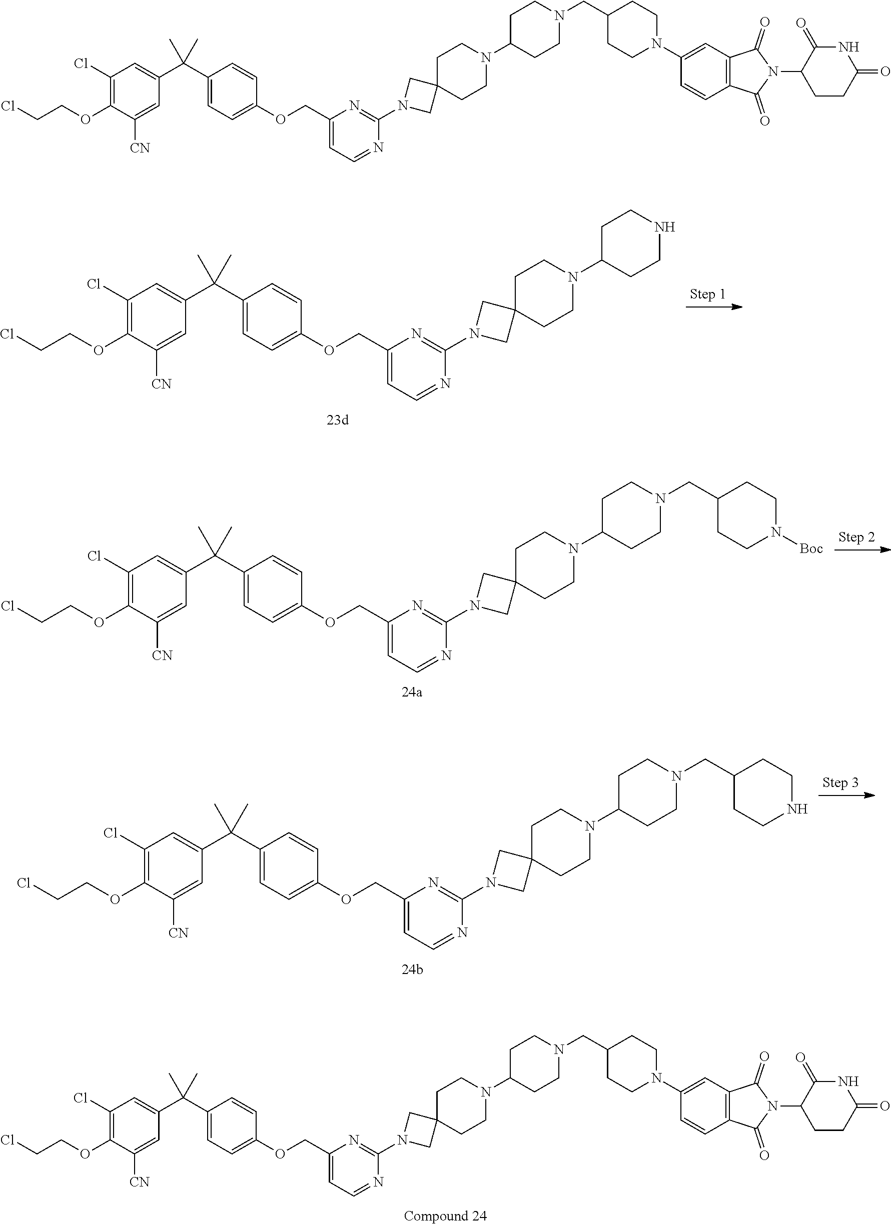

### Step 1: tert-butyl 4-((4-(2-(4-((4-(2-(3-chloro-4-(2-chloroethoxy)-5-cyanophenyl)propan-2-yl)phenoxy)methyl)pyrimidin-2-yl)-2,7-diazaspiro[3.5]nonan-7-yl)piperidin-1-yl)methyl) piperidine-1-carboxylate (24a)

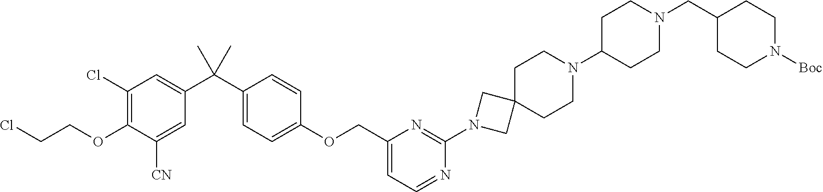 [SMILES: CC(C)(C)OC(=O)N1CCC(CN2CCC(N3CCC4(CC3)CN(c3nccc(COc5ccc(C(C)(C)c6cc(Cl)c(OCCCl)c(C#N)c6)cc5)n3)C4)CC2)CC1]

The above crude 23d trifluoroacetate (185 mg) was dissolved in 15 mL of DCE, and solid sodium bicarbonate (74 mg, 0.88 mmol) was added. The mixture was stirred at room temperature for 20 min, and then tert-butyl 4-formylpiperidine-1-carboxylate (91 mg, 0.46 mmol) and 0.05 mL of acetic acid were successively added. The mixture was stirred at room temperature for 60 min, and then sodium triacetoxyborohydride (94 mg, 0.44 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 24a (180 mg, two-step yield from compound 23c: >99%).

LCMS m/z=846.4 [M+1]⁺

### Step 2: 3-chloro-2-(2-chloroethoxy)-5-(2-(4-((2-(7-(1-(piperidin-4-ylmethyl)piperidin-4-yl)-2,7-diazaspiro[3.5]nonan-2-yl)pyrimidin-4-yl)methoxy)phenyl)propan-2-yl)benzonitrile (24b) trifluoroacetate

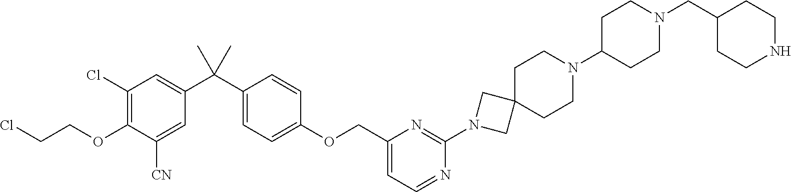 [SMILES: CC(C)(c1ccc(OCc2ccnc(N3CC4(CCN(C5CCN(CC6CCNCC6)CC5)CC4)C3)n2)cc1)c1cc(Cl)c(OCCCl)c(C#N)c1]

24a (50 mg, 0.059 mmol) was dissolved in 5 mL of DCM, and 2 mL of trifluoroacetic acid was added. The mixture was stirred at room temperature for 2 h. The reaction liquid was concentrated under reduced pressure, to afford crude 24b trifluoroacetate (60 mg).

LC-MS m/z=746.4 [M+1]⁺

### Step 3: 3-chloro-2-(2-chloroethoxy)-5-(2-(4-((2-(7-(1-((1-(2-(2,6-dioxopiperidin-3-yl)-1,3-dioxoisoindolin-5-yl)piperidin-4-yl)methyl)piperidin-4-yl)-2,7-diazaspiro[3.5]nonan-2-yl)pyrimidin-4-yl)methoxy)phenyl)propan-2-yl)benzonitrile (Compound 24)

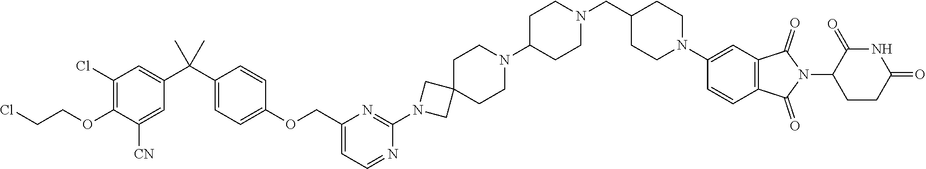 [SMILES: CC(C)(c1ccc(OCc2ccnc(N3CC4(CCN(C5CCN(CC6CCN(c7ccc8c(c7)C(=O)N(C7CCC(=O)NC7=O)C8=O)CC6)CC5)CC4)C3)n2)cc1)c1cc(Cl)c(OCCCl)c(C#N)c1]

The above crude 24b trifluoroacetate (60 mg) was dissolved in 4 mL of DMSO, and solid sodium bicarbonate (25 mg, 0.30 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 24 (25 mg, two-step yield from compound 24a: 42%).

¹H NMR (400 MHz, CDCl₃) δ 8.32 (d, 1H), 7.67 (d, 1H), 7.44 (d, 1H), 7.31 (d, 1H), 7.29-7.24 (m, 2H), 7.14-6.98 (m, 3H), 6.93-6.83 (m, 2H), 6.79 (m, 1H), 4.99-4.87 (m, 3H), 4.42 (t, 2H), 4.00-3.70 (m, 8H), 3.15-2.55 (m, 16H), 2.30-2.20 (m, 2H), 2.18-2.09 (m, 1H), 2.04-1.97 (m, 2H), 1.94-1.83 (m, 4H), 1.83-1.68 (m, 3H), 1.64 (s, 6H), 1.33-1.21 (m, 2H).

LCMS m/z=501.9 [M/2+1]⁺.

### Example 25: 3-chloro-2-(2-chloroethoxy)-5-(2-(4-((2-(7-(1-((1-(2-(2,6-dioxopiperidin-3-yl)-1,3-dioxoisoindolin-4-yl)piperidin-4-yl)methyl)piperidin-4-yl)-2,7-diazaspiro[3.5]nonan-2-yl)pyrimidin-4-yl)methoxy)phenyl)propan-2-yl)benzonitrile (Compound 25)

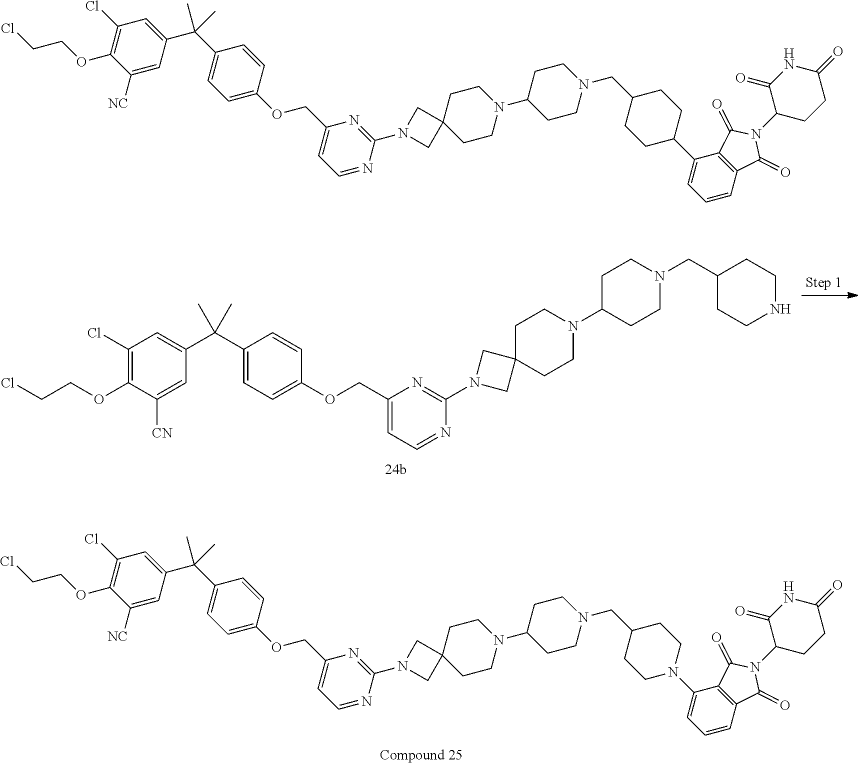 [SMILES: CC(C)(c1ccc(OCc2ccnc(N3CC4(CCN(C5CCN(CC6CCC(c7cccc8c7C(=O)N(C7CCC(=O)NC7=O)C8=O)CC6)CC5)CC4)C3)n2)cc1)c1cc(Cl)c(OCCCl)c(C#N)c1 ; CC(C)(c1ccc(OCc2ccnc(N3CC4(CCN(C5CCN(CC6CCNCC6)CC5)CC4)C3)n2)cc1)c1cc(Cl)c(OCCCl)c(C#N)c1 ; CC(C)(c1ccc(OCc2ccnc(N3CC4(CCN(C5CCN(CC6CCN(c7cccc8c7C(=O)N(C7CCC(=O)NC7=O)C8=O)CC6)CC5)CC4)C3)n2)cc1)c1cc(Cl)c(OCCCl)c(C#N)c1]

The above crude 24b trifluoroacetate (60 mg) was dissolved in 4 mL of DMSO, and solid sodium bicarbonate (25 mg, 0.30 mmol) was added. The mixture was stirred at room temperature for 10 min, and then 0.2 mL of DIPEA and 2-(2,6-dioxopiperidin-3-yl)-4-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 25 (20 mg, two-step yield from compound 24a: 34%).

¹H NMR (400 MHz, CDCl₃) δ 8.31 (d, 1H), 7.97 (s, 1H), 7.61-7.51 (m, 1H), 7.44 (d, 1H), 7.36 (d, 1H), 7.31 (d, 1H), 7.17 (d, 1H), 7.13-7.05 (m, 2H), 6.92-6.84 (m, 2H), 6.81-6.73 (m, 1H), 5.00-4.90 (m, 3H), 4.40-3.64 (m, 8H), 3.15-2.42 (m, 12H), 2.32-2.17 (m, 2H), 2.17-2.06 (m, 2H), 2.06-1.76 (m, 8H), 1.74-1.52 (m, 11H), 1.52-1.38 (m, 3H).

LCMS m/z=1002.3 [M+1]⁺.

### Example 26: 3-chloro-2-(2-chloroethoxy)-5-(2-(4-((2-(7-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)-2,7-diazaspiro[3.5]nonan-2-yl)pyrimidin-4-yl)methoxy)phenyl)propan-2-yl)benzonitrile (Compound 26)

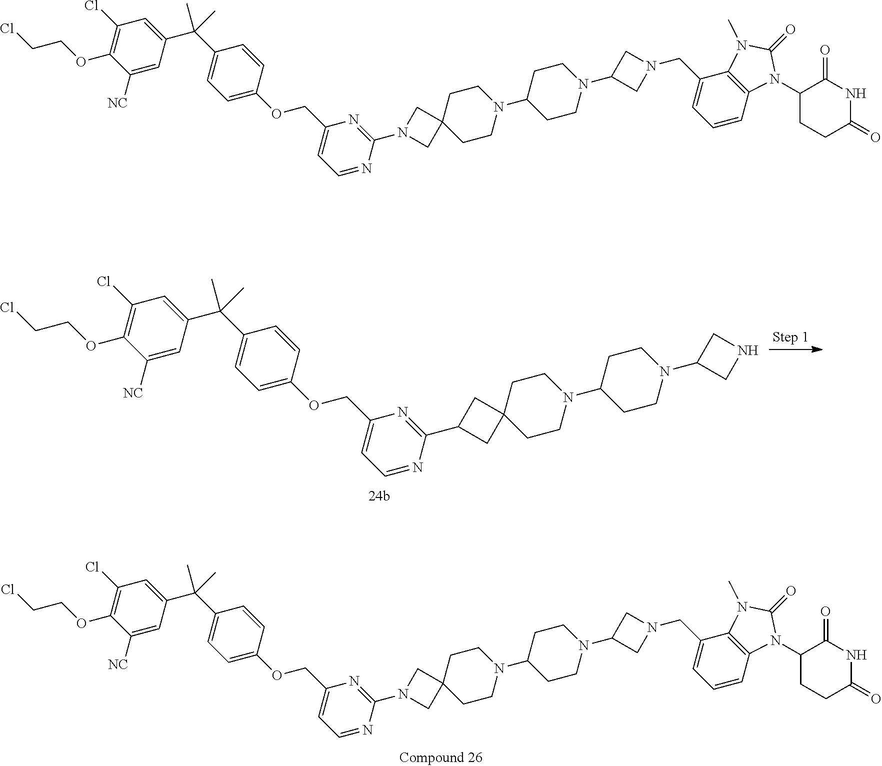 [SMILES: Cn1c(=O)n(C2CCC(=O)NC2=O)c2cccc(CN3CC(N4CCC(N5CCC6(CC5)CN(c5nccc(COc7ccc(C(C)(C)c8cc(Cl)c(OCCCl)c(C#N)c8)cc7)n5)C6)CC4)C3)c21 ; CC(C)(c1ccc(OCc2ccnc(C3CC4(CCN(C5CCN(C6CNC6)CC5)CC4)C3)n2)cc1)c1cc(Cl)c(OCCCl)c(C#N)c1 ; Cn1c(=O)n(C2CCC(=O)NC2=O)c2cccc(CN3CC(N4CCC(N5CCC6(CC5)CN(c5nccc(COc7ccc(C(C)(C)c8cc(Cl)c(OCCCl)c(C#N)c8)cc7)n5)C6)CC4)C3)c21]

The above crude 24b trifluoroacetate (60 mg) was dissolved in 4 mL of anhydrous THF, and solid sodium bicarbonate (25 mg, 0.30 mmol) was added. The mixture was stirred at room temperature for 10 min, and then 1-(2,6-dioxopiperidin-3-yl)-3-methyl-2-oxo-2,3-dihydro-1H-benzo[d]imidazole-4-carbaldehyde (see WO 2020113233 for the synthetic method) (40 mg, 0.14 mmol) and acetic acid (0.02 mL, 0.35 mmol) were successively added. The mixture was stirred at room temperature for 2 h, and then sodium triacetoxyborohydride (26 mg, 0.12 mmol) was added. The resulting mixture was reacted at room temperature for 16 h. To the reaction liquid was added solid sodium bicarbonate (100 mg, 1.19 mmol), and the mixture was stirred at room temperature for 15 min and then filtered. The filtrate was concentrated under reduced pressure, and the resulting crude product was separated and purified by a thick preparative plate (methanol/dichloromethane (v/v)=1:12), to afford compound 26 (15 mg, two-step yield from compound 24a: 26%).

¹H NMR (400 MHz, DMSO-d₆) δ 10.07 (s, 1H), 8.32 (d, 1H), 7.62 (d, 1H), 7.56 (d, 1H), 7.23-7.12 (m, 2H), 7.09-7.00 (m, 1H), 7.00-6.87 (m, 4H), 6.72 (d, 1H), 5.36 (dd, 1H), 4.96 (s, 2H), 4.47-4.37 (m, 2H), 3.99-3.91 (m, 2H), 3.86-3.66 (m, 6H), 3.63 (s, 3H), 3.38-3.30 (m, 2H), 2.97-2.78 (m, 4H), 2.78-2.57 (m, 5H), 2.57-2.51 (m, 2H), 2.49-2.35 (m, 2H), 2.35-2.06 (m, 2H), 2.06-1.95 (m, 1H), 1.86-1.66 (m, 6H), 1.62 (s, 6H), 1.52-1.36 (m, 2H).

LCMS m/z=975.4 [M+1]⁺

### Example 27

### 3-chloro-2-(2-chloroethoxy)-5-(2-(4-((2-(9-(1-((1-(2-(2,6-dioxopiperidin-3-yl)-1,3-dioxoisoindolin-5-yl)piperidin-4-yl)methyl)piperidin-4-yl)-3,9-diazaspiro[5.5]undecan-3-yl)pyrimidin-4-yl)methoxy)phenyl)propan-2-yl)benzonitrile (Compound 27)

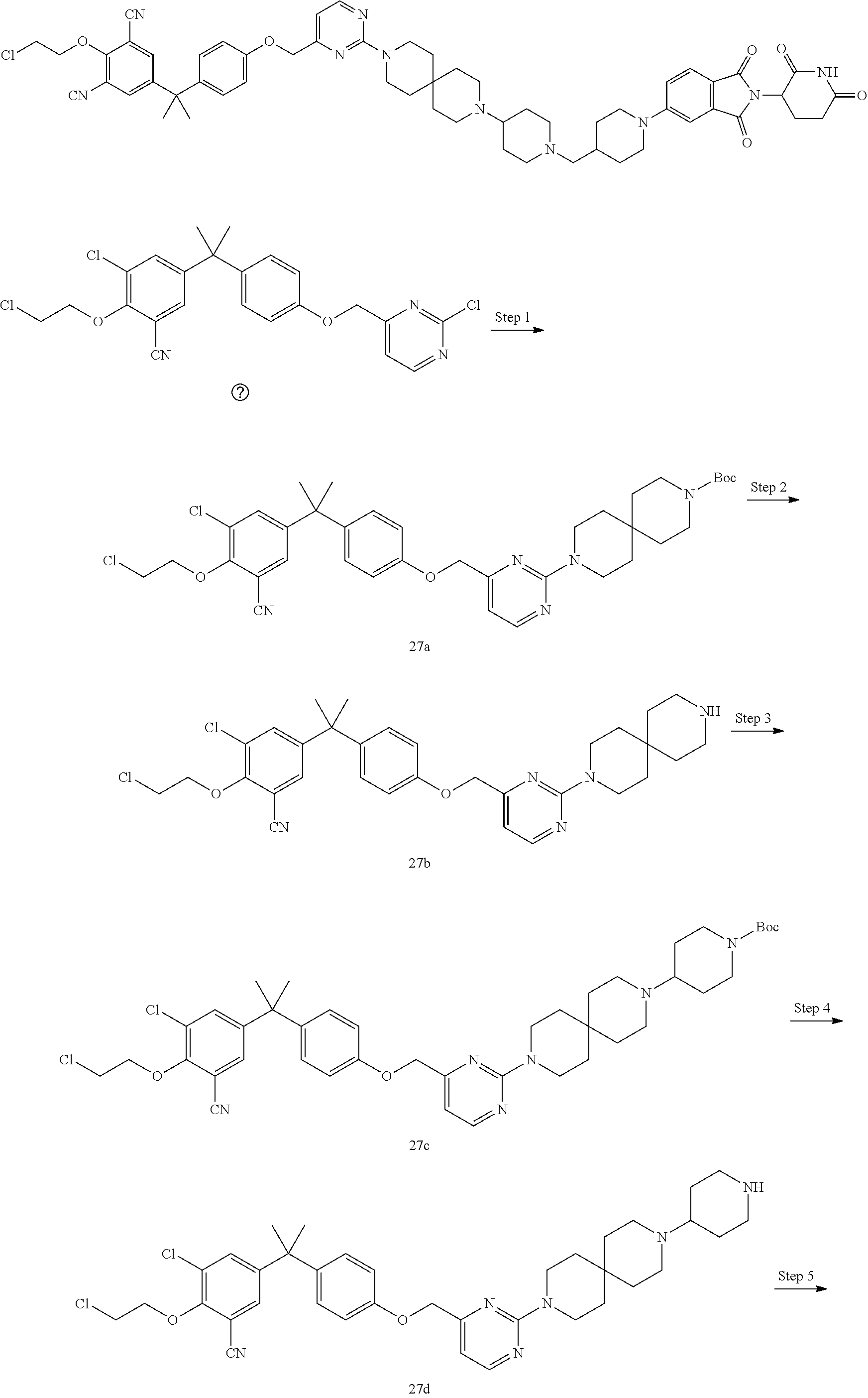

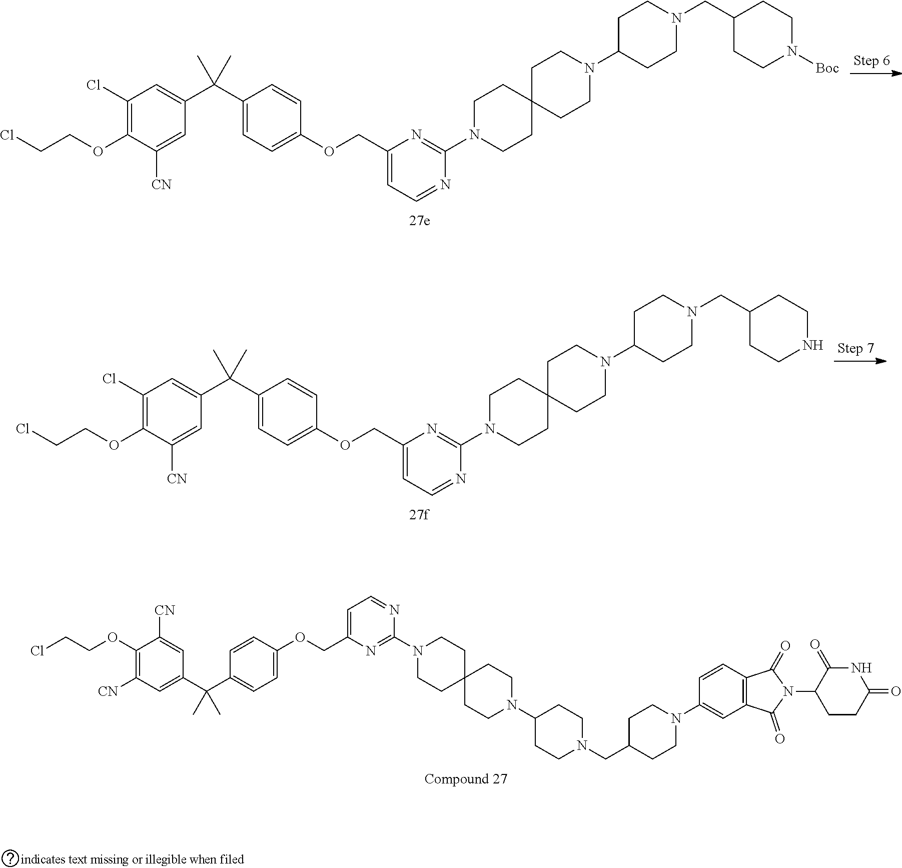

### Step 1: tert-butyl 9-(4-((4-(2-(3-chloro-4-(2-chloroethoxy)-5-cyanophenyl)propan-2-yl)phenoxy)methyl)pyrimidin-2-yl)-3,9-diazaspiro[5.5]undecane-3-carboxylate (27a)

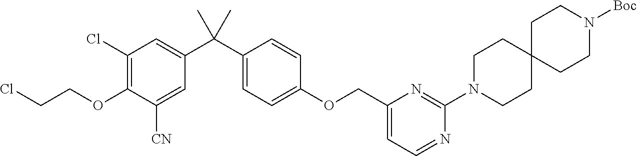 [SMILES: CC(C)(C)OC(=O)N1CCC2(CC1)CCN(c1nccc(COc3ccc(C(C)(C)c4cc(Cl)c(OCCCl)c(C#N)c4)cc3)n1)CC2]

3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (350 mg, 0.73 mmol) was dissolved in 20 mL of DMF, and tert-butyl 3,9-diazaspiro[5.5]undecane-3-carboxylate (320 mg, 1.26 mmol) and potassium bicarbonate (147 mg, 1.47 mmol) were successively added. The mixture was warmed to 60° C. and reacted for 3 h. The mixture was cooled to room temperature, and to the reaction system was added 50 mL of water. The mixture was extracted with ethyl acetate (40 mL×3), and the organic phase was washed with water (50 mL×2), dried over anhydrous sodium sulfate, and concentrated under reduced pressure. The crude product was separated and purified by silica gel column chromatography (petroleum ether/ethyl acetate (v/v)=2:1), to afford 27a (480 mg, yield: 95%).

LCMS m/z=694.2 [M+1]⁺

### Step 2: 5-(2-(4-((2-(3,9-diazaspiro[5.5]undecan-3-yl)pyrimidin-4-yl)methoxy)phenyl) propan-2-yl)-3-chloro-2-(2-chloroethoxy)benzonitrile (27b) trifluoroacetate

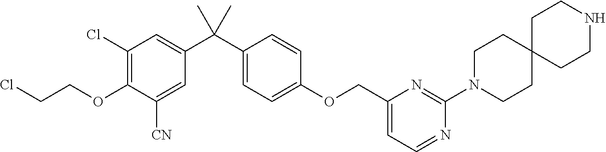 [SMILES: CC(C)(c1ccc(OCc2ccnc(N3CCC4(CCNCC4)CC3)n2)cc1)c1cc(Cl)c(OCCCl)c(C#N)c1]

27a (0.25 g, 0.36 mmol) was dissolved in 10 mL of DCM, and 4 mL of trifluoroacetic acid was added. The mixture was stirred at room temperature for 2 h. The reaction liquid was concentrated under reduced pressure, to afford crude 27b trifluoroacetate (0.29 g).

LCMS m/z=594.2 [M+1]⁺

### Step 3: tert-butyl 4-(9-(4-((4-(2-(3-chloro-4-(2-chloroethoxy)-5-cyanophenyl)propan-2-yl)phenoxy)methyl)pyrimidin-2-yl)-3,9-diazaspiro[5.5]undecan-3-yl)piperidine-1-carboxylate (27c)

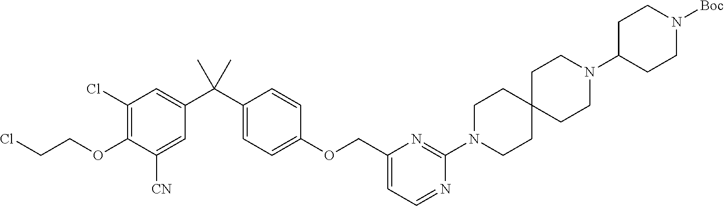 [SMILES: CC(C)(C)OC(=O)N1CCC(N2CCC3(CCN(c4nccc(COc5ccc(C(C)(C)c6cc(Cl)c(OCCCl)c(C#N)c6)cc5)n4)CC3)CC2)CC1]

The crude 27b trifluoroacetate (0.29 g) was dissolved in 15 mL of DCE, and solid sodium bicarbonate (91 mg, 1.08 mmol) was added. The mixture was stirred at room temperature for 20 min, and then N-Boc-4-piperidone (143 mg, 0.72 mmol) and 0.1 mL of acetic acid were added. The mixture was stirred at room temperature for 60 min, and then sodium triacetoxyborohydride (153 mg, 0.72 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 27c (270 mg, two-step yield from compound 27a: 97%).

LCMS m/z=777.3 [M+1]⁺

### Step 4: 3-chloro-2-(2-chloroethoxy)-5-(2-(4-((2-(9-(piperidin-4-yl)-3,9-diazaspiro[5.5]undecan-3-yl)pyrimidin-4-yl)methoxy)phenyl)propan-2-yl)benzonitrile (27d) trifluoroacetate

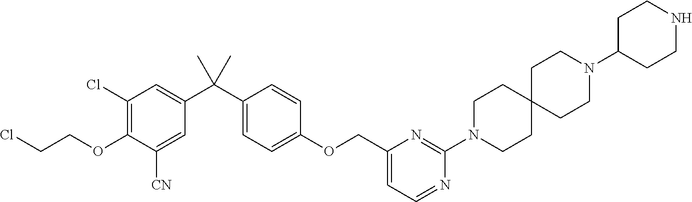 [SMILES: CC(C)(c1ccc(OCc2ccnc(N3CCC4(CC3)CCN(C3CCNCC3)CC4)n2)cc1)c1cc(Cl)c(OCCCl)c(C#N)c1]

27c (100 mg, 0.13 mmol) was dissolved in 10 mL of DCM, and 4 mL of trifluoroacetic acid was added. The mixture was stirred at room temperature for 2 h. The reaction liquid was concentrated under reduced pressure, to afford crude 27d trifluoroacetate (120 mg).

LCMS m/z=677.3 [M+1]⁺

### Step 5: tert-butyl 4-((4-(9-(4-((4-(2-(3-chloro-4-(2-chloroethoxy)-5-cyanophenyl)propan-2-yl)phenoxy)methyl)pyrimidin-2-yl)-3,9-diazaspiro[5.5]undecan-3-yl)piperidin-1-yl)methyl) piperidine-1-carboxylate (27e)

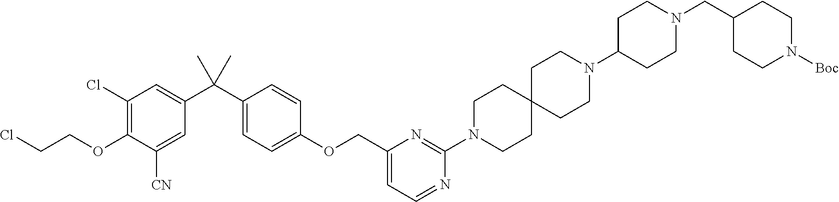 [SMILES: CC(C)(C)OC(=O)N1CCC(CN2CCC(N3CCC4(CCN(c5nccc(COc6ccc(C(C)(C)c7cc(Cl)c(OCCCl)c(C#N)c7)cc6)n5)CC4)CC3)CC2)CC1]

The above crude 27d trifluoroacetate (120 mg) was dissolved in 15 mL of DCE, and solid sodium bicarbonate (45 mg, 0.54 mmol) was added. The mixture was stirred at room temperature for 20 min, and then tert-butyl 4-formylpiperidine-1-carboxylate (55 mg, 0.26 mmol) and 0.05 mL of acetic acid were successively added. The mixture was stirred at room temperature for 60 min, and then sodium triacetoxyborohydride (55 mg, 0.26 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 27e (110 mg, two-step yield from compound 27c: 97%).

LCMS m/z=874.4 [M+1]⁺

### Step 6: 3-chloro-2-(2-chloroethoxy)-5-(2-(4-((2-(9-(1-(piperidin-4-ylmethyl)piperidin-4-yl)-3,9-diazaspiro[5.5]undecan-3-yl)pyrimidin-4-yl)methoxy)phenyl)propan-2-yl)benzonitrile (27f) trifluoroacetate

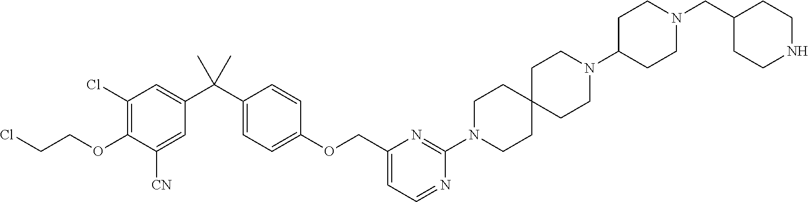 [SMILES: CC(C)(c1ccc(OCc2ccnc(N3CCC4(CC3)CCN(C3CCN(CC5CCNCC5)CC3)CC4)n2)cc1)c1cc(Cl)c(OCCCl)c(C#N)c1]

27e (100 mg, 0.11 mmol) was dissolved in 5 mL of DCM, and 2 mL of trifluoroacetic acid was added. The mixture was stirred at room temperature for 2 h. The reaction liquid was concentrated under reduced pressure, to afford crude 27f trifluoroacetate (120 mg).

LC-MS m/z=774.4 [M+1]⁺

### Step 7: 3-chloro-2-(2-chloroethoxy)-5-(2-(4-((2-(9-(1-((1-(2-(2,6-dioxopiperidin-3-yl)-1,3-dioxoisoindolin-5-yl)piperidin-4-yl)methyl)piperidin-4-yl)-3,9-diazaspiro[5.5]undecan-3-yl)pyrimidin-4-yl)methoxy)phenyl)propan-2-yl)benzonitrile (Compound 27)

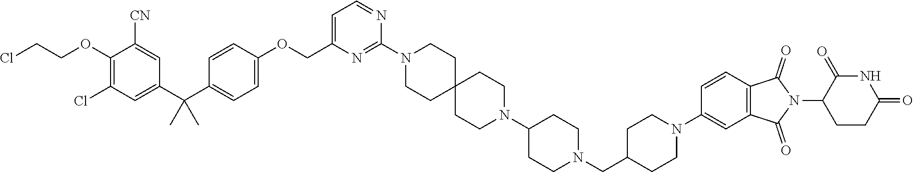 [SMILES: CC(C)(c1ccc(OCc2ccnc(N3CCC4(CC3)CCN(C3CCN(CC5CCN(c6ccc7c(c6)C(=O)N(C6CCC(=O)NC6=O)C7=O)CC5)CC3)CC4)n2)cc1)c1cc(Cl)c(OCCCl)c(C#N)c1]

The above crude 27f trifluoroacetate (60 mg) was dissolved in 4 mL of DMSO, and solid sodium bicarbonate (25 mg, 0.30 mmol) was added. The mixture was stirred at room temperature for 10 min, and 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 27 (25 mg, two-step yield from compound 27e: 44%).

¹H NMR (400 MHz, CDCl₃) δ 8.30 (d, 1H), 8.07 (br.s, 1H), 7.66 (d, 1H), 7.44 (d, 1H), 7.31 (d, 1H), 7.29-7.26 (m, 1H), 7.15-6.97 (m, 3H), 6.94-6.83 (m, 2H), 6.68 (d, 1H), 4.99-4.87 (m, 3H), 4.42 (t, 2H), 4.00-3.72 (m, 8H), 3.05-2.37 (m, 12H), 2.25-2.07 (m, 3H), 2.04-1.82 (m, 7H), 1.82-1.60 (m, 12H), 1.60-1.47 (m, 4H), 1.33-1.20 (m, 2H).

LCMS m/z=1030.3 [M+1]⁺.

### Example 28

### 3-chloro-2-(2-chloroethoxy)-5-(2-(4-((2-(9-(1-((1-(2-(2,6-dioxopiperidin-3-yl)-1,3-dioxoisoindolin-4-yl)piperidin-4-yl)methyl)piperidin-4-yl)-3,9-diazaspiro[5.5]undecan-3-yl)pyrimidin-4-yl)methoxy)phenyl)propan-2-yl)benzonitrile (Compound 28)

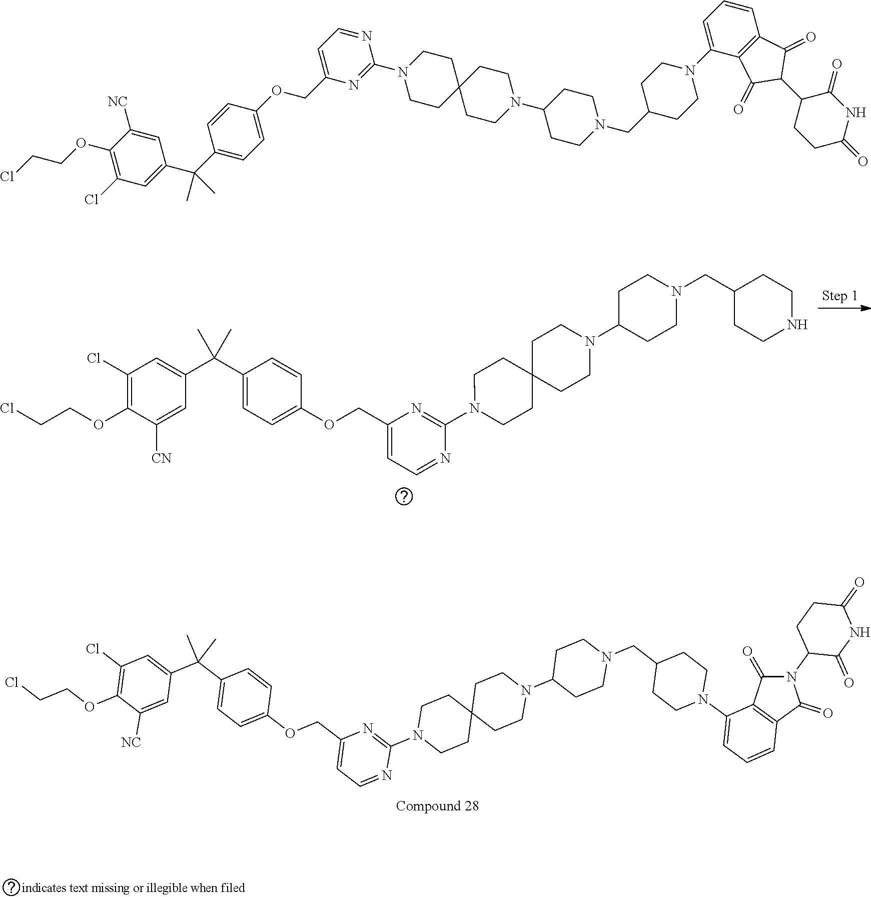 [SMILES: CC(C)(c1ccc(OCc2ccnc(N3CCC4(CC3)CCN(C3CCN(CC5CCN(c6cccc7c6C(=O)C(C6CCC(=O)NC6=O)C7=O)CC5)CC3)CC4)n2)cc1)c1cc(Cl)c(OCCCl)c(C#N)c1 ; CC(C)(c1ccc(OCc2ccnc(N3CCC4(CC3)CCN(C3CCN(CC5CCNCC5)CC3)CC4)n2)cc1)c1cc(Cl)c(OCCCl)c(C#N)c1 ; CC(C)(c1ccc(OCc2ccnc(N3CCC4(CC3)CCN(C3CCN(CC5CCN(c6cccc7c6C(=O)N(C6CCC(=O)NC6=O)C7=O)CC5)CC3)CC4)n2)cc1)c1cc(Cl)c(OCCCl)c(C#N)c1]

The above crude 27f trifluoroacetate (60 mg) was dissolved in 4 mL of DMSO, and solid sodium bicarbonate (25 mg, 0.30 mmol) was added. The mixture was stirred at room temperature for 10 min, and 0.2 mL of DIPEA and 2-(2,6-dioxopiperidin-3-yl)-4-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The resulting crude product was separated and purified by a thick preparative plate (methanol/dichloromethane (v/v)=1:12), to afford compound 28 (25 mg, two-step yield from compound 27e: 44%).

¹H NMR (400 MHz, CDCl₃) δ 8.29 (d, 1H), 8.04 (br.s, 1H), 7.60-7.50 (m, 1H), 7.44 (d, 1H), 7.35 (d, 1H), 7.32 (d, 1H), 7.17 (d, 1H), 7.13-7.04 (m, 2H), 6.92-6.83 (m, 2H), 6.67 (d, 1H), 5.00-4.90 (m, 3H), 4.41 (t, 2H), 3.92-3.66 (m, 8H), 3.05-2.32 (m, 12H), 2.30-2.17 (m, 2H), 2.16-2.06 (m, 1H), 2.04-1.80 (m, 7H), 1.79-1.59 (m, 12H), 1.59-1.49 (m, 4H), 1.49-1.37 (m, 2H).

LCMS m/z=1030.4 [M+1]⁺.

### Example 29

### 3-chloro-2-(2-chloroethoxy)-5-(2-(4-((2-(9-(1-((1-(2,6-dioxopiperidin-3-yl)-3-methyl-2-oxo-2,3-dihydro-1H-benzo[d]imidazol-4-yl)methyl)azetidin-3-yl)-3,9-diazaspiro[5.5]undecan-3-yl)pyrimidin-4-yl)methoxy)phenyl)propan-2-yl)benzonitrile (Compound 29) trifluoroacetate

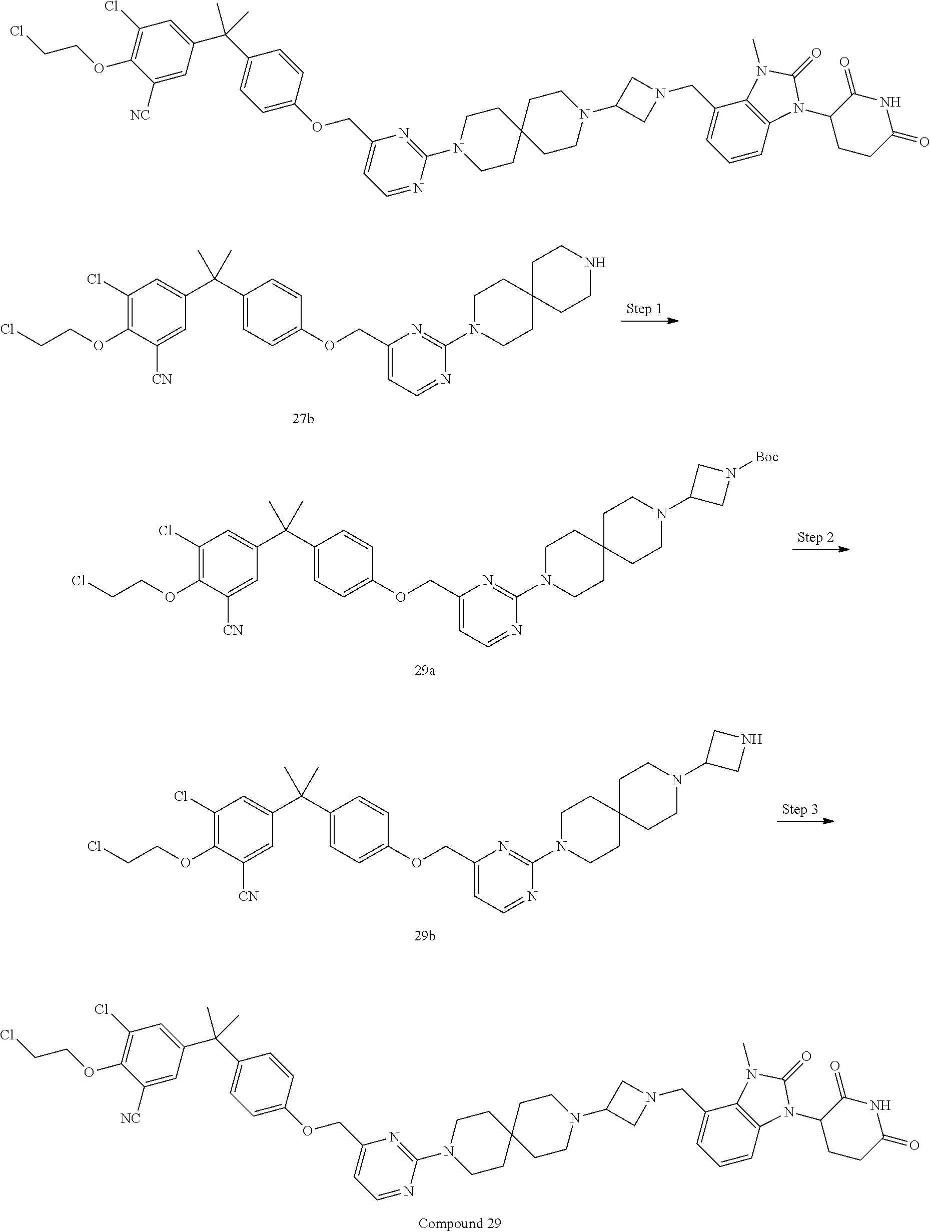 [SMILES: Cn1c(=O)n(C2CCC(=O)NC2=O)c2cccc(CN3CC(N4CCC5(CCN(c6nccc(COc7ccc(C(C)(C)c8cc(Cl)c(OCCCl)c(C#N)c8)cc7)n6)CC5)CC4)C3)c21 ; CC(C)(c1ccc(OCc2ccnc(N3CCC4(CCNCC4)CC3)n2)cc1)c1cc(Cl)c(OCCCl)c(C#N)c1 ; CC(C)(C)OC(=O)N1CC(N2CCC3(CCN(c4nccc(COc5ccc(C(C)(C)c6cc(Cl)c(OCCCl)c(C#N)c6)cc5)n4)CC3)CC2)C1 ; CC(C)(c1ccc(OCc2ccnc(N3CCC4(CC3)CCN(C3CNC3)CC4)n2)cc1)c1cc(Cl)c(OCCCl)c(C#N)c1 ; Cn1c(=O)n(C2CCC(=O)NC2=O)c2cccc(CN3CC(N4CCC5(CCN(c6nccc(COc7ccc(C(C)(C)c8cc(Cl)c(OCCCl)c(C#N)c8)cc7)n6)CC5)CC4)C3)c21]

### Step 1: tert-butyl 3-(9-(4-((4-(2-(3-chloro-4-(2-chloroethoxy)-5-cyanophenyl)propan-2-yl)phenoxy)methyl)pyrimidin-2-yl)-3,9-diazaspiro[5.5]undecan-3-yl)azetidine-1-carboxylate (29a)

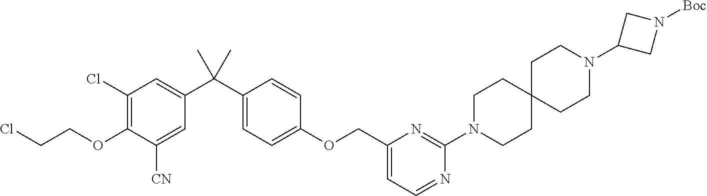 [SMILES: CC(C)(C)OC(=O)N1CC(N2CCC3(CCN(c4nccc(COc5ccc(C(C)(C)c6cc(Cl)c(OCCCl)c(C#N)c6)cc5)n4)CC3)CC2)C1]

The 27b trifluoroacetate (0.12 g) was dissolved in 15 mL of DCE, and solid sodium bicarbonate (36 mg, 0.43 mmol) was added. The mixture was stirred at room temperature for 20 min, and then N-Boc-3-azetidinone (50 mg, 0.29 mmol) and 0.05 mL of acetic acid were successively added. The mixture was stirred at room temperature for 60 min, and then sodium triacetoxyborohydride (61 mg, 0.29 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 29a (110 mg, two-step yield from compound 27a: 99%).

LCMS m/z=749.3 [M+1]⁺

### Step 2: 5-(2-(4-((2-(9-(azetidin-3-yl)-3,9-diazaspiro[5.5]undecan-3-yl)pyrimidin-4-yl)methoxy)phenyl)propan-2-yl)-3-chloro-2-(2-chloroethoxy)benzonitrile (29b) trifluoroacetate

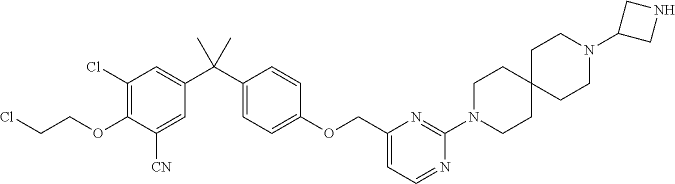 [SMILES: CC(C)(c1ccc(OCc2ccnc(N3CCC4(CC3)CCN(C3CNC3)CC4)n2)cc1)c1cc(Cl)c(OCCCl)c(C#N)c1]

29a (50 mg, 0.067 mmol) was dissolved in 5 mL of DCM, and 2 mL of trifluoroacetic acid was added. The mixture was stirred at room temperature for 2 h. The reaction liquid was concentrated under reduced pressure, to afford crude 29b trifluoroacetate (65 mg).

LCMS m/z=649.3 [M+1]⁺

### Step 3: 3-chloro-2-(2-chloroethoxy)-5-(2-(4-((2-(9-(1-((1-(2,6-dioxopiperidin-3-yl)-3-methyl-2-oxo-2,3-dihydro-1H-benzo[d]imidazol-4-yl)methyl)azetidin-3-yl)-3,9-diazaspiro[5.5]undecan-3-yl)pyrimidin-4-yl)methoxy)phenyl)propan-2-yl)benzonitrile (Compound 29) trifluoroacetate

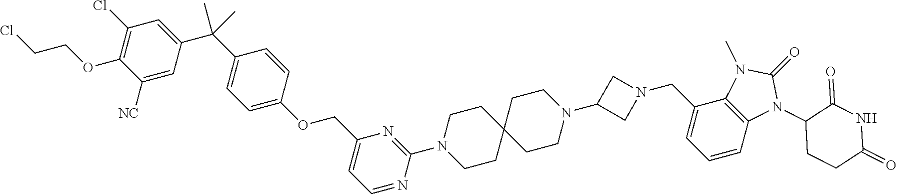 [SMILES: Cn1c(=O)n(C2CCC(=O)NC2=O)c2cccc(CN3CC(N4CCC5(CCN(c6nccc(COc7ccc(C(C)(C)c8cc(Cl)c(OCCCl)c(C#N)c8)cc7)n6)CC5)CC4)C3)c21]

The above crude 29b trifluoroacetate (65 mg) was dissolved in 4 mL of anhydrous THF, and solid sodium bicarbonate (25 mg, 0.30 mmol) was added. The mixture was stirred at room temperature for 10 min, and then 1-(2,6-dioxopiperidin-3-yl)-3-methyl-2-oxo-2,3-dihydro-1H-benzo[d]imidazole-4-carbaldehyde (see WO 2020113233 for the synthetic method) (40 mg, 0.14 mmol) and acetic acid (0.02 mL, 0.35 mmol) were successively added. The mixture was stirred at room temperature for 2 h, and then sodium triacetoxyborohydride (28 mg, 0.13 mmol) was added. The mixture was reacted at room temperature for 16 h, and then solid sodium bicarbonate (100 mg, 1.19 mmol) was added. The resulting mixture was stirred at room temperature for 15 min and then filtered. The filtrate was concentrated under reduced pressure, and the crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 29 trifluoroacetate (15 mg).

¹H NMR (400 MHz, DMSO-d₆) δ 11.10 (s, 1H), 8.34 (d, 1H), 7.61 (d, 1H), 7.56 (d, 1H), 7.28-6.89 (m, 7H), 6.67 (d, 1H), 5.41 (dd, 1H), 4.97 (s, 2H), 4.53-4.28 (m, 4H), 4.24-3.84 (m, 7H), 3.82-3.68 (m, 4H), 3.62 (s, 3H), 3.25-2.96 (m, 4H), 2.96-2.82 (m, 1H), 2.81-2.57 (m, 2H), 2.07-1.93 (m, 1H), 1.93-1.40 (m, 14H).

LCMS m/z=920.2 [M+1]⁺.

### Example 30

### 3-chloro-2-(2-chloroethoxy)-5-(2-(4-((2-(9-(1-(1-(2-(2,6-dioxopiperidin-3-yl)-1,3-dioxoisoindolin-5-yl)azetidin-3-yl)piperidin-4-yl)-3,9-diazaspiro[5.5]undecan-3-yl)pyrimidin-4-yl)methoxy)phenyl)propan-2-yl)benzonitrile (Compound 30) trifluoroacetate

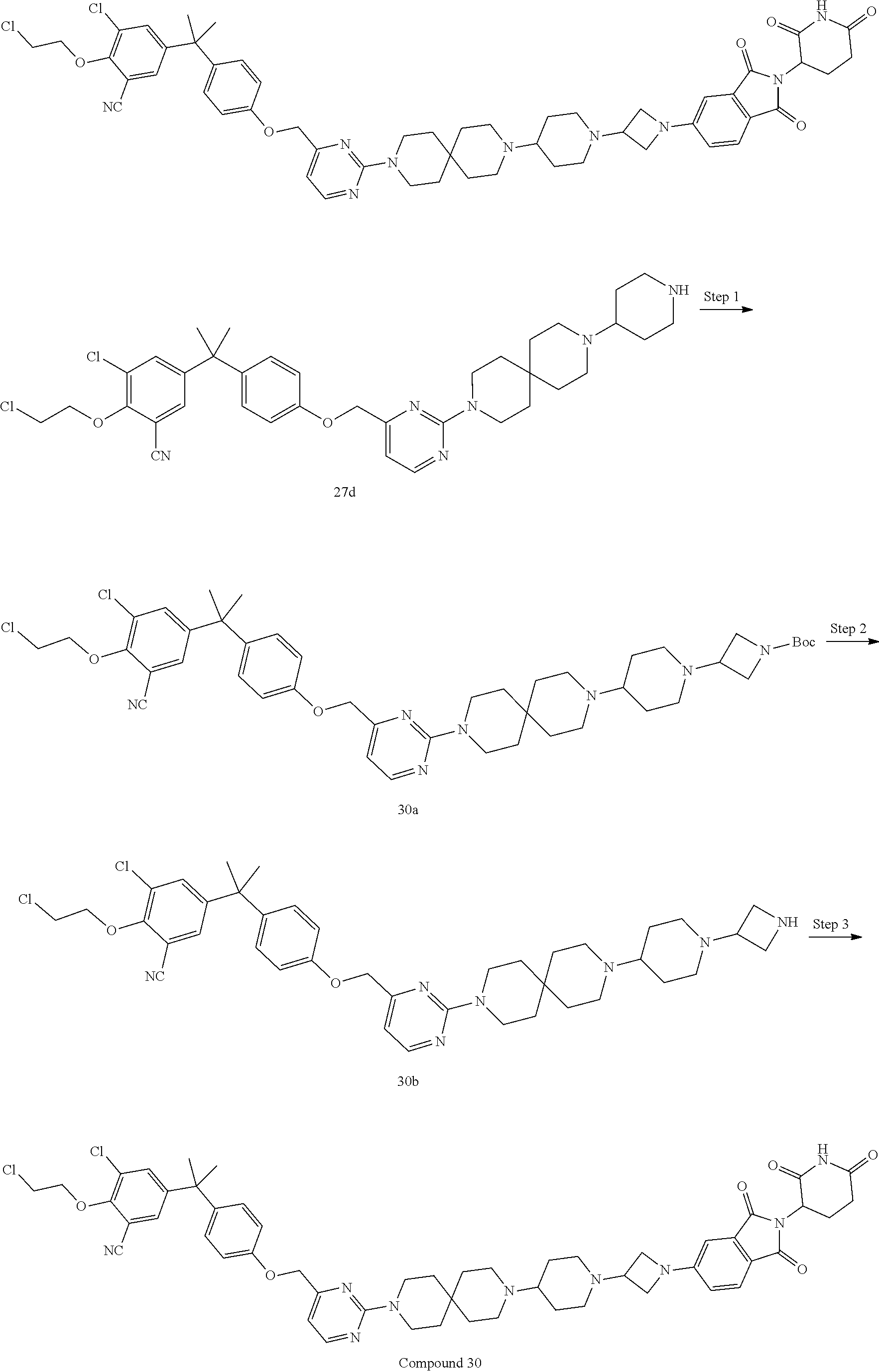

### Step 1: tert-butyl 3-(4-(9-(4-((4-(2-(3-chloro-4-(2-chloroethoxy)-5-cyanophenyl)propan-2-yl)phenoxy)methyl)pyrimidin-2-yl)-3,9-diazaspiro[5.5]undecan-3-yl)piperidin-yl)azetidine-1-carboxylate (30a)

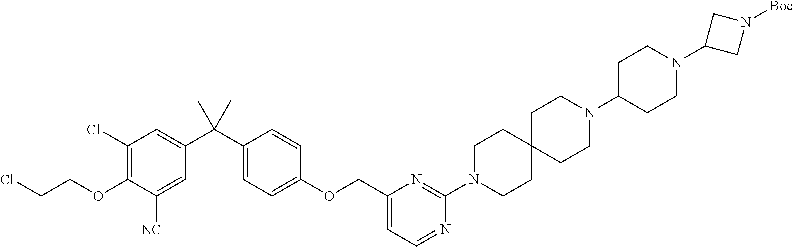 [SMILES: CC(C)(C)OC(=O)N1CC(N2CCC(N3CCC4(CCN(c5nccc(COc6ccc(C(C)(C)c7cc(Cl)c(OCCCl)c(C#N)c7)cc6)n5)CC4)CC3)CC2)C1]

The above crude 27d trifluoroacetate (190 mg) was dissolved in 15 mL of DCE, and solid sodium bicarbonate (64 mg, 0.76 mmol) was added. The mixture was stirred at room temperature for 20 m, and then 1-Boc-3-azetidinone (66 mg, 0.39 mmol) and 0.05 mL of acetic acid were successively added. The mixture was stirred at room temperature for 60 min, and then sodium triacetoxyborohydride (81 mg, 0.38 mmol) was added. The resulting mixture was reacted at room temperature for 16 h. To the reaction system was slowly added 30 mL of saturated sodium bicarbonate solution, and the mixture was extracted with 120 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 30a (160 mg, two-step yield from compound 27c: 94%).

LCMS m/z=832.3 [M+1]⁺

### Step 2: 5-(2-(4-((2-(9-(1-(azetidin-3-yl)piperidin-4-yl)-3,9-diazaspiro[5.5]undecan-3-yl)pyrimidin-4-yl)methoxy)phenyl)propan-2-yl)-3-chloro-2-(2-chloroethoxy)benzonitrile (30b) trifluoroacetate

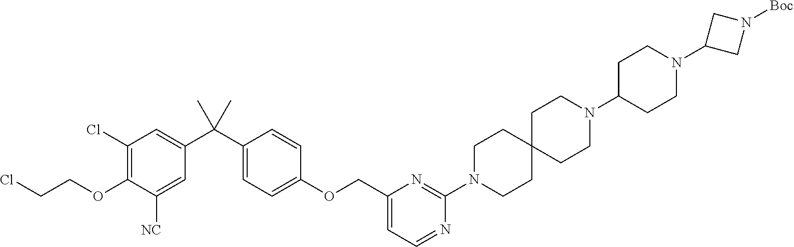 [SMILES: CC(C)(C)OC(=O)N1CC(N2CCC(N3CCC4(CCN(c5nccc(COc6ccc(C(C)(C)c7cc(Cl)c(OCCCl)c(C#N)c7)cc6)n5)CC4)CC3)CC2)C1]

30a (50 mg, 0.07 mmol) was dissolved in 5 mL of DCM, and 2 mL of trifluoroacetic acid was added. The mixture was stirred at room temperature for 2 h. The reaction liquid was concentrated under reduced pressure, to afford crude 30b trifluoroacetate (60 mg).

LC-MS m/z=732.3 [M+1]⁺

### Step 3: 3-chloro-2-(2-chloroethoxy)-5-(2-(4-((2-(9-(1-(1-(2-(2,6-dioxopiperidin-3-yl)-1,3-dioxoisoindolin-5-yl)azetidin-3-yl)piperidin-4-yl)-3,9-diazaspiro[5.5]undecan-3-yl)pyrimidin-4-yl)methoxy)phenyl)propan-2-yl)benzonitrile (Compound 30) trifluoroacetate

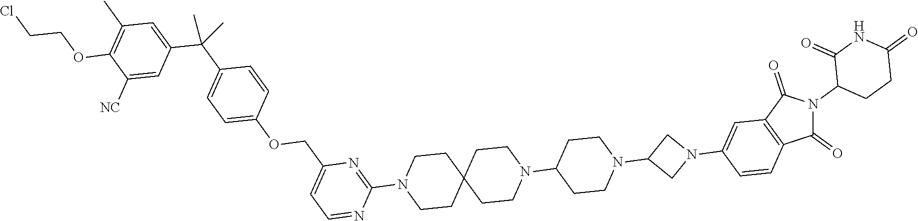 [SMILES: Cc1cc(C(C)(C)c2ccc(OCc3ccnc(N4CCC5(CC4)CCN(C4CCN(C6CN(c7ccc8c(c7)C(=O)N(C7CCC(=O)NC7=O)C8=O)C6)CC4)CC5)n3)cc2)cc(C#N)c1OCCCl]

The above crude 30b trifluoroacetate (60 mg) was dissolved in 4 mL of DMSO, and solid sodium bicarbonate (25 mg, 0.30 mmol) was added. The mixture was stirred at room temperature for 10 min, and 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 30 trifluoroacetate (15 mg).

¹H NMR (400 MHz, DMSO-d₆) δ 11.07 (s, 1H), 8.35 (d, 1H), 7.72 (d, 1H), 7.65-7.53 (m, 2H), 7.22-7.10 (m, 2H), 6.98-6.90 (m, 2H), 6.90-6.85 (m, 1H), 6.79-6.70 (m, 1H), 6.67 (d, 1H), 5.07 (dd, 1H), 4.97 (s, 2H), 4.45-4.37 (m, 2H), 4.36-4.26 (m, 2H), 4.25-4.15 (m, 2H), 4.15-4.04 (m, 1H), 4.00-3.91 (m, 2H), 3.85-3.67 (m, 4H), 3.48-3.30 (m, 4H), 3.22-3.00 (m, 3H), 2.97-2.68 (m, 3H), 2.65-2.52 (m, 2H), 2.43-2.28 (m, 2H), 2.12-1.76 (m, 5H), 1.75-1.50 (m, 10H), 1.46-1.32 (m, 2H).

LCMS m/z=988.3 [M+1]⁺.

### Example 31

### 3-chloro-2-(2-chloroethoxy)-5-(2-(4-((2-(9-(1-(1-((1-(2,6-dioxopiperidin-3-yl)-3-methyl-2-oxo-2,3-dihydro-1H-benzo[d]imidazol-4-yl)methyl)azetidin-3-yl)piperidin-4-yl)-3,9-diazaspiro[5.5]undecan-3-yl)pyrimidin-4-yl)methoxy)phenyl)propan-2-yl)benzonitrile (Compound 31) trifluoroacetate

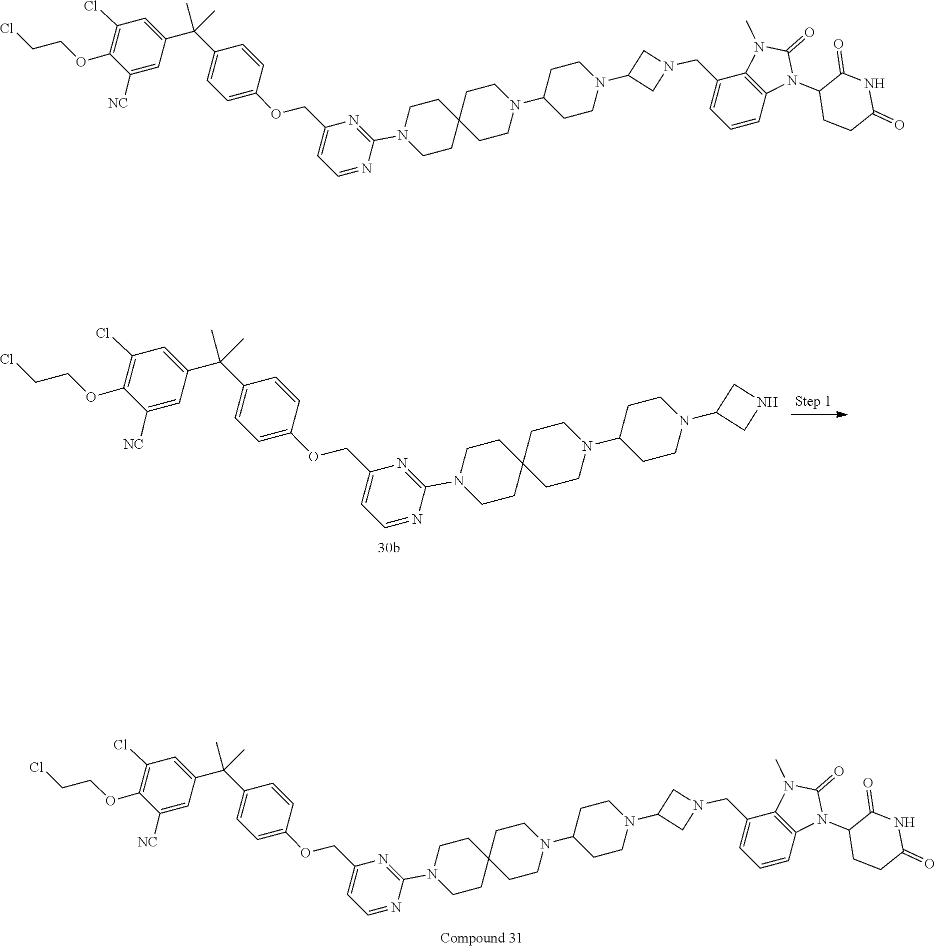

The above crude 30b trifluoroacetate (60 mg) was dissolved in 4 mL of anhydrous THF, and solid sodium bicarbonate (25 mg, 0.30 mmol) was added. The mixture was stirred at room temperature for 10 min, and then 1-(2,6-dioxopiperidin-3-yl)-3-methyl-2-oxo-2,3-dihydro-1H-benzo[d]imidazole-4-carbaldehyde (see WO 2020113233 for the synthetic method) (40 mg, 0.14 mmol) and acetic acid (0.02 mL, 0.35 mmol) were successively added. The mixture was stirred at room temperature for 2 h, and then sodium triacetoxyborohydride (26 mg, 0.12 mmol) was added. The mixture was reacted at room temperature for 16 h, and then solid sodium bicarbonate (100 mg, 1.19 mmol) was added. The resulting mixture was stirred at room temperature for 15 min and then filtered. The filtrate was concentrated under reduced pressure, and the crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 31 trifluoroacetate (10 mg).

¹H NMR (400 MHz, DMSO-d₆) δ 11.11 (s, 1H), 8.35 (d, 1H), 7.61 (d, 1H), 7.57 (d, 1H), 7.30-7.05 (m, 5H), 7.04-6.88 (m, 2H), 6.67 (d, 1H), 5.42 (dd, 1H), 4.97 (s, 2H), 4.74-4.60 (m, 2H), 4.45-4.38 (m, 2H), 4.23-4.09 (m, 2H), 4.09-3.98 (m, 2H), 3.98-3.91 (m, 2H), 3.82-3.68 (m, 4H), 3.60 (s, 3H), 3.52-3.32 (m, 3H), 3.32-3.18 (m, 1H), 3.17-3.02 (m, 2H), 2.98-2.83 (m, 1H), 2.80-2.58 (m, 2H), 2.54-2.50 (m, 4H), 2.25-1.85 (m, 7H), 1.76-1.50 (m, 10H), 1.44-1.30 (m, 2H).

LCMS m/z=1003.4 [M+1]⁺.

### Example 32: 3-chloro-5-(2-(4-((2-(4-((1′-(2-(2,6-dioxopiperidin-3-yl)-1,3-dioxoisoindolin-5-yl)-[1,4′-bipiperidin]-4-yl)methyl)piperazin-1-yl)pyrimidin-4-yl)methoxy)phenyl)propan-2-yl)benzonitrile (Compound 32)

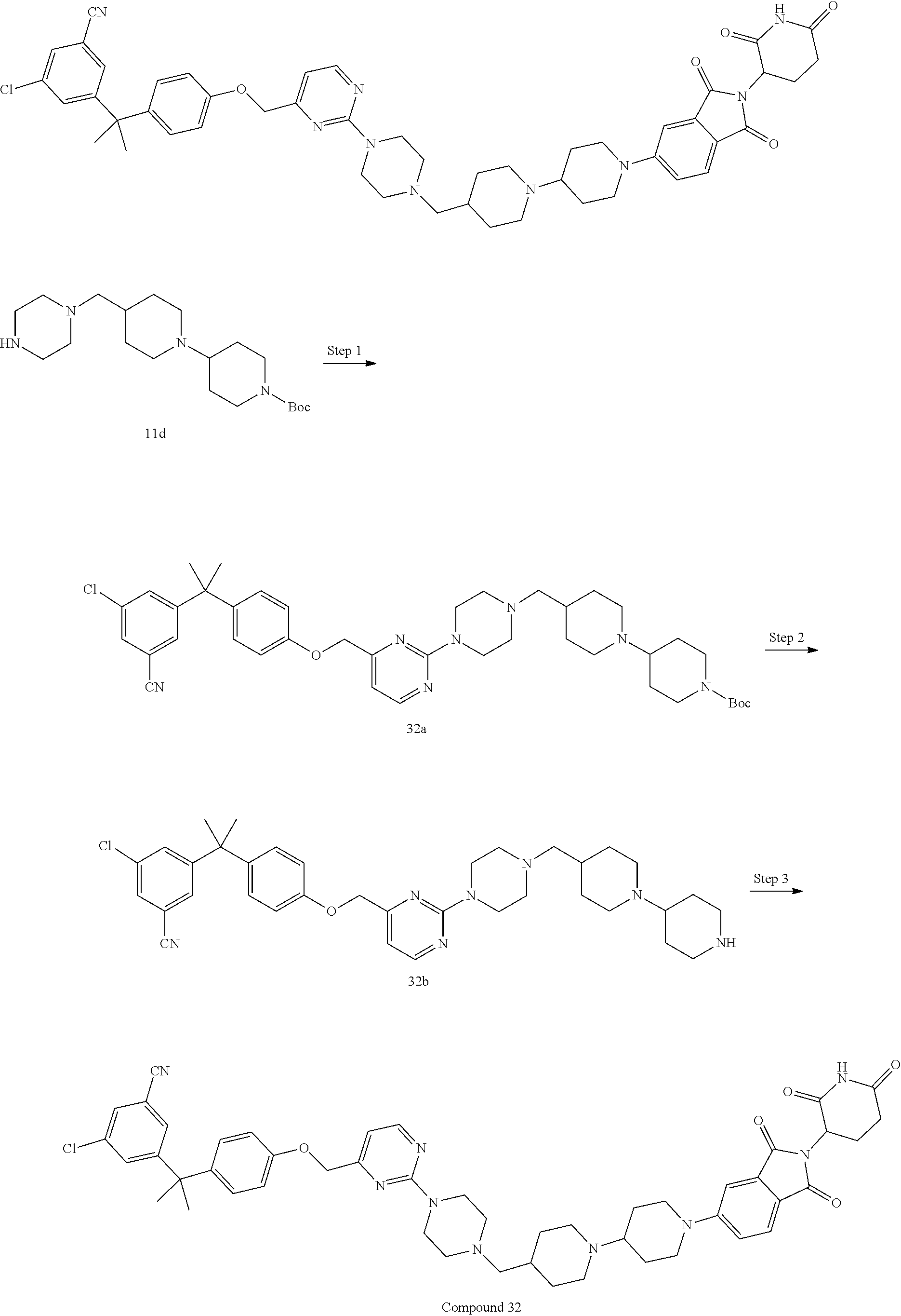 [SMILES: CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)cc(C#N)c1 ; CC(C)(C)OC(=O)N1CCC(N2CCC(CN3CCNCC3)CC2)CC1 ; CC(C)(C)OC(=O)N1CCC(N2CCC(CN3CCN(c4nccc(COc5ccc(C(C)(C)c6cc(Cl)cc(C#N)c6)cc5)n4)CC3)CC2)CC1 ; CC(C)(c1ccc(OCc2ccnc(N3CCN(CC4CCN(C5CCNCC5)CC4)CC3)n2)cc1)c1cc(Cl)cc(C#N)c1 ; CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)cc(C#N)c1]

### Step 1: tert-butyl 4-((4-(4-((4-(2-(3-chloro-5-cyanophenyl)propan-2-yl)phenoxy)methyl)pyrimidin-2-yl)piperazin-1-yl)methyl)-[1,4′-bipiperidine]-1′-carboxylate (32a)

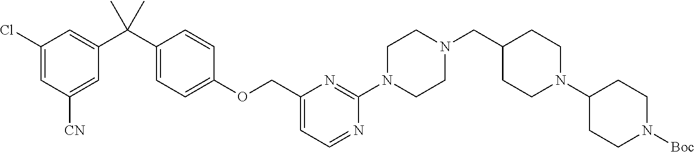 [SMILES: CC(C)(C)OC(=O)N1CCC(N2CCC(CN3CCN(c4nccc(COc5ccc(C(C)(C)c6cc(Cl)cc(C#N)c6)cc5)n4)CC3)CC2)CC1]

3-chloro-5-(2-(4-((2-chloropyrimidin-4-yl)methoxy)phenyl)propan-2-yl)benzonitrile (9d) (250 mg, 0.63 mmol) was dissolved in 15 mL of DMF, and solid potassium bicarbonate (105 mg, 1.05 mmol) and the above crude tert-butyl 4-(piperazin-1-ylmethyl)-[1,4′-bipiperidine]-1′-carboxylate (11d) (250 mg) were successively added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 32a (300 mg, yield from compound 9d: 65%).

LCMS m/z=728.3 [M+1]⁺

### Step 2: 3-(2-(4-((2-(4-([1,4′-bipiperidin]-4-ylmethyl)piperazin-1-yl)pyrimidin-4-yl)methoxy)phenyl)propan-2-yl)-5-chlorobenzonitrile (32b) trifluoroacetate

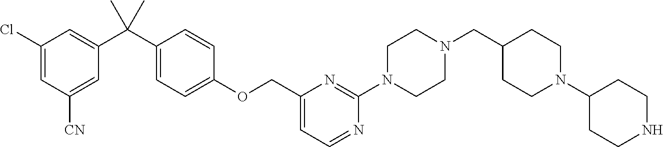 [SMILES: CC(C)(c1ccc(OCc2ccnc(N3CCN(CC4CCN(C5CCNCC5)CC4)CC3)n2)cc1)c1cc(Cl)cc(C#N)c1]

32a (60 mg, 0.08 mmol) was dissolved in 5 mL of DCM, and 2 mL of trifluoroacetic acid was added. The mixture was stirred at room temperature for 2 h. The reaction liquid was concentrated under reduced pressure, to afford crude 32b trifluoroacetate (70 mg).

LCMS m/z=628.3 [M+1]⁺

### Step 3: 3-chloro-5-(2-(4-((2-(4-((1′-(2-(2,6-dioxopiperidin-3-yl)-1,3-dioxoisoindolin-5-yl)-[1,4′-bipiperidin]-4-yl)methyl)piperazin-1-yl)pyrimidin-4-yl)methoxy)phenyl)propan-2-yl)benzonitrile (Compound 32)

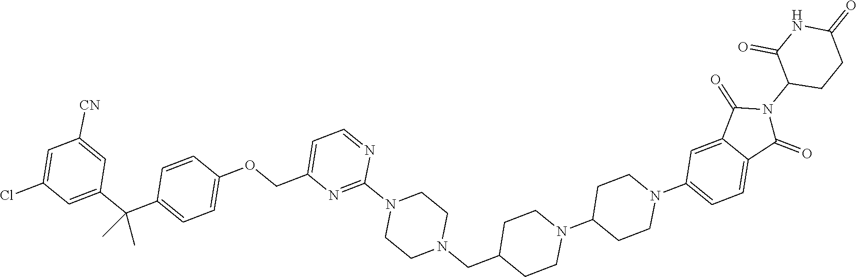 [SMILES: CC(C)(c1ccc(OCc2ccnc(N3CCN(CC4CCN(C5CCN(c6ccc7c(c6)C(=O)N(C6CCC(=O)NC6=O)C7=O)CC5)CC4)CC3)n2)cc1)c1cc(Cl)cc(C#N)c1]

The above crude 32b trifluoroacetate (70 mg) was dissolved in 4 mL of DMSO, and solid sodium bicarbonate (32 mg, 0.38 mmol) was added. The mixture was stirred at room temperature for 10 min, and then 0.5 mL of DIPEA and 2-(2,6-dioxopiperidin-3-yl)-5-fluoroisoindoline-1,3-dione (see WO 2017197056 for the synthetic method) (41 mg, 0.15 mmol) were added. The resulting mixture was warmed to 80° C. and reacted for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=15:1), to afford compound 32 (20 mg, two-step yield from compound 32a: 28%).

¹H NMR (400 MHz, CDCl₃) δ 8.30 (d, 1H), 8.08 (br.s, 1H), 7.67 (d, 1H), 7.46-7.41 (m, 2H), 7.39-7.35 (m, 1H), 7.31-7.26 (m, 1H), 7.13-7.00 (m, 3H), 6.94-6.83 (m, 2H), 6.70 (d, 1H), 5.00-4.88 (m, 3H), 4.06-3.94 (m, 2H), 3.88-3.76 (m, 4H), 3.07-2.93 (m, 4H), 2.93-2.65 (m, 3H), 2.61-2.51 (m, 1H), 2.50-2.39 (m, 4H), 2.28-2.16 (m, 4H), 2.16-2.08 (m, 1H), 2.02-1.90 (m, 2H), 1.89-1.76 (m, 2H), 1.73-1.60 (m, 8H), 1.60-1.46 (m, 1H), 1.34-1.18 (m, 2H).

LCMS m/z=442.8 [M/2+1]⁺

### Example 33

### 3-chloro-5-(2-(4-((2-(4-((1-(2-(2,6-dioxopiperidin-3-yl)-1,3-dioxoisoindolin-5-yl)piperidin-4-yl)methyl)piperazin-1-yl)pyrimidin-4-yl)methoxy)phenyl)propan-2-yl)benzonitrile (Compound 33)

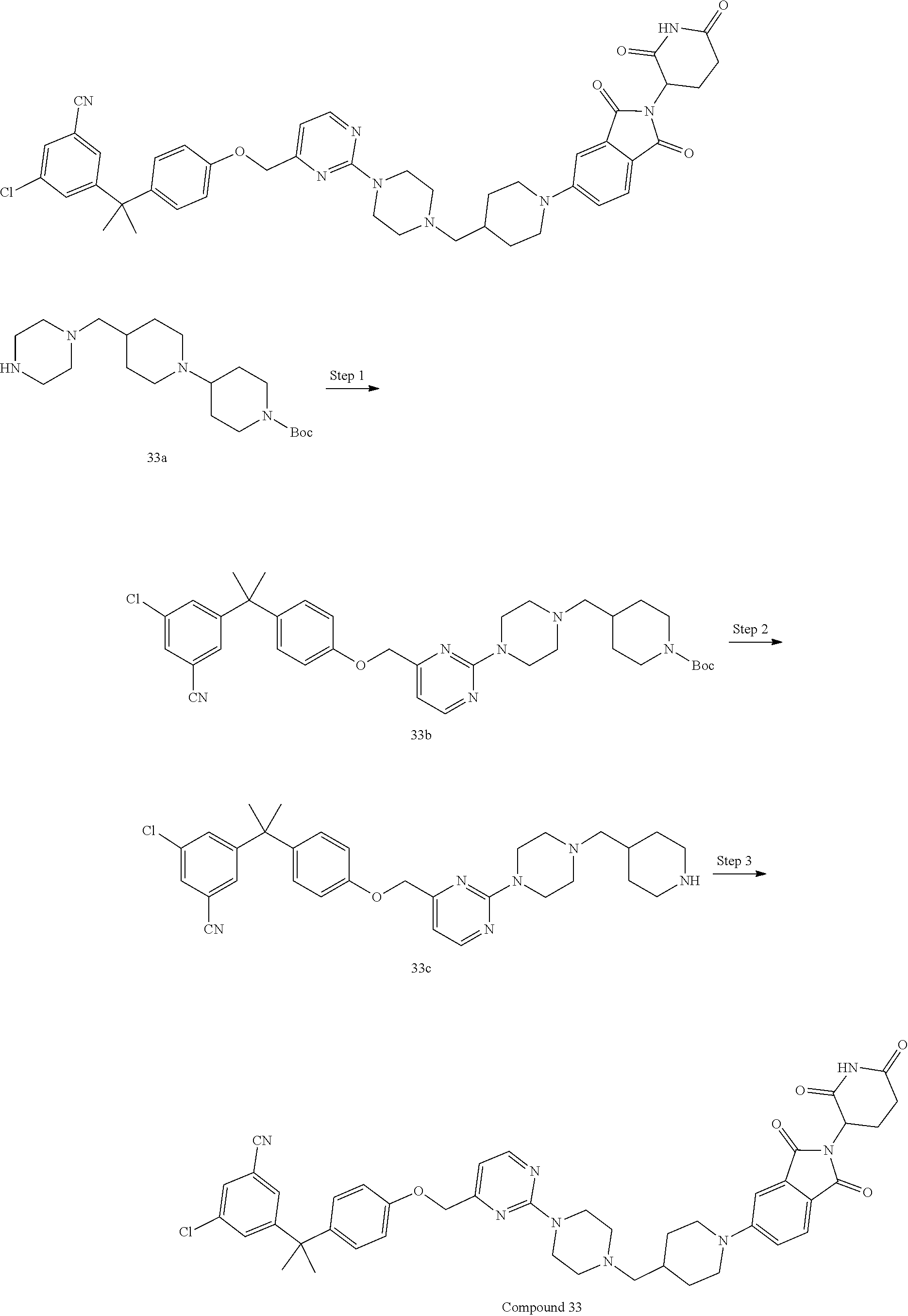 [SMILES: CC(C)(c1ccc(OCc2ccnc(N3CCN(CC4CCN(c5ccc6c(c5)C(=O)N(C5CCC(=O)NC5=O)C6=O)CC4)CC3)n2)cc1)c1cc(Cl)cc(C#N)c1 ; CC(C)(C)OC(=O)N1CCC(N2CCC(CN3CCNCC3)CC2)CC1 ; CC(C)(C)OC(=O)N1CCC(CN2CCN(c3nccc(COc4ccc(C(C)(C)c5cc(Cl)cc(C#N)c5)cc4)n3)CC2)CC1 ; CC(C)(c1ccc(OCc2ccnc(N3CCN(CC4CCNCC4)CC3)n2)cc1)c1cc(Cl)cc(C#N)c1 ; CC(C)(c1ccc(OCc2ccnc(N3CCN(CC4CCN(c5ccc6c(c5)C(=O)N(C5CCC(=O)NC5=O)C6=O)CC4)CC3)n2)cc1)c1cc(Cl)cc(C#N)c1]

### Step 1: tert-butyl 4-((4-(4-((4-(2-(3-chloro-5-cyanophenyl)propan-2-yl)phenoxy)methyl)pyrimidin-2-yl)piperazin-1-yl)methyl)piperidine-1-carboxylate (33b)

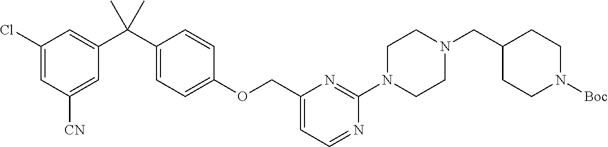 [SMILES: CC(C)(C)OC(=O)N1CCC(CN2CCN(c3nccc(COc4ccc(C(C)(C)c5cc(Cl)cc(C#N)c5)cc4)n3)CC2)CC1]

3-chloro-5-(2-(4-((2-chloropyrimidin-4-yl)methoxy)phenyl)propan-2-yl)benzonitrile (9d) (250 mg, 0.63 mmol) was dissolved in 15 ml of DMF, and solid potassium bicarbonate (105 mg, 1.05 mmol) and tert-butyl 4-(piperazin-1-ylmethyl)piperidine-1-carboxylate (33a) (see WO 2015086693 for the synthetic method) (250 mg, 0.882 mmol) were successively added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 33b (300 mg, yield from compound 9d: 74%).

LCMS m/z=645.3 [M+1]⁺

### Step 2: 3-chloro-5-(2-(4-((2-(4-(piperidin-4-ylmethyl)piperazin-1-yl)pyrimidin-4-yl)methoxy)phenyl)propan-2-yl)benzonitrile (33c) trifluoroacetate

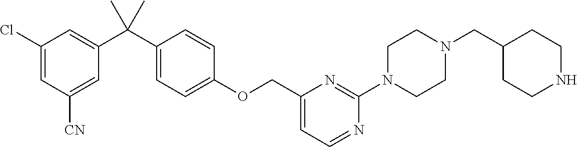 [SMILES: CC(C)(c1ccc(OCc2ccnc(N3CCN(CC4CCNCC4)CC3)n2)cc1)c1cc(Cl)cc(C#N)c1]

33b (60 mg, 0.09 mmol) was dissolved in 5 mL of DCM, and 2 mL of trifluoroacetic acid was added. The mixture was stirred at room temperature for 2 h. The reaction liquid was concentrated under reduced pressure, to afford crude 33c trifluoroacetate (70 mg).

LCMS m/z=545.3 [M+1]⁺

### Step 3: 3-chloro-5-(2-(4-((2-(4-((1-(2-(2,6-dioxopiperidin-3-yl)-1,3-dioxoisoindolin-5-yl)piperidin-4-yl)methyl)piperazin-1-yl)pyrimidin-4-yl)methoxy)phenyl)propan-2-yl)benzonitrile (Compound 33)

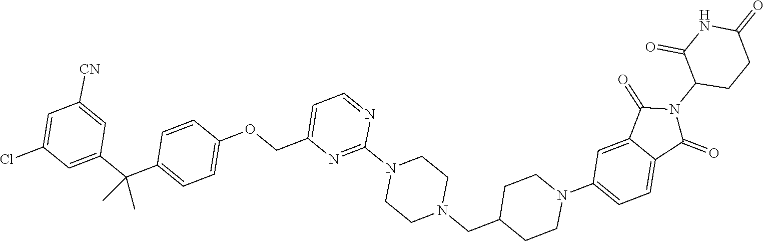 [SMILES: CC(C)(c1ccc(OCc2ccnc(N3CCN(CC4CCN(c5ccc6c(c5)C(=O)N(C5CCC(=O)NC5=O)C6=O)CC4)CC3)n2)cc1)c1cc(Cl)cc(C#N)c1]

The above crude 33c trifluoroacetate (70 mg) was dissolved in 4 mL of DMSO, and solid sodium bicarbonate (32 mg, 0.38 mmol) was added. The mixture was stirred at room temperature for 10 min, and then 0.5 mL of DIPEA and 2-(2,6-dioxopiperidin-3-yl)-5-fluoroisoindoline-1,3-dione (see WO 2017197056 for the synthetic method) (41 mg, 0.15 mmol) were added. The resulting mixture was warmed to 80° C. and stirred for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=15:1), to afford compound 33 (28 mg, two-step yield from compound 33b: 39%).

¹H NMR (400 MHz, CDCl₃) δ 8.31 (d, 1H), 8.04 (s, 1H), 7.67 (d, 1H), 7.47-7.41 (m, 2H), 7.40-7.34 (m, 1H), 7.29 (d, 1H), 7.14-7.01 (m, 3H), 6.92-6.84 (m, 2H), 6.71 (d, 1H), 5.01-4.89 (m, 3H), 4.05-3.70 (m, 6H), 3.07-2.93 (m, 2H), 2.93-2.65 (m, 3H), 2.56-2.39 (m, 4H), 2.35-2.19 (m, 2H), 2.19-2.07 (m, 1H), 2.00-1.89 (m, 2H), 1.89-1.75 (m, 1H), 1.65 (s, 6H), 1.38-1.24 (m, 2H).

LCMS m/z=801.3 [M+1]⁺

### Example 34

### 3-chloro-5-(2-(4-((2-(4-(1-(7-(2-(2,6-dioxopiperidin-3-yl)-1,3-dioxoisoindolin-5-yl)-7-azaspiro[3.5]nonan-2-yl)piperidin-4-yl)piperazin-1-yl)pyrimidin-4-yl)methoxy)phenyl) propan-2-yl)benzonitrile (Compound 34) trifluoroacetate

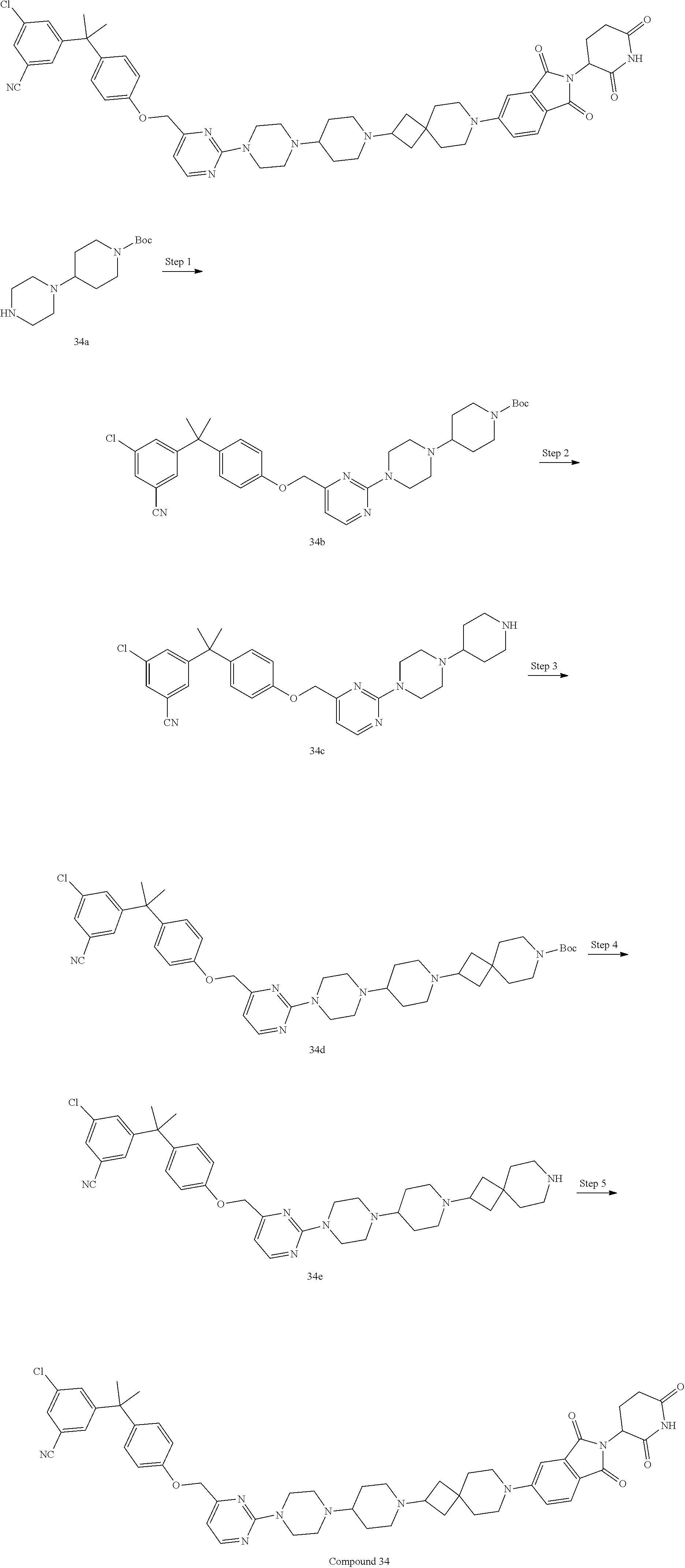

### Step 1: tert-butyl 4-(4-(4-((4-(2-(3-chloro-5-cyanophenyl)propan-2-yl)phenoxy)methyl)pyrimidin-2-yl)piperazin-1-yl)piperidine-1-carboxylate (34b)

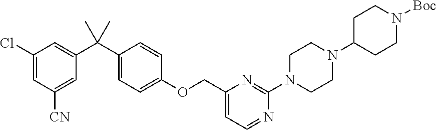 [SMILES: CC(C)(C)OC(=O)N1CCC(N2CCN(c3nccc(COc4ccc(C(C)(C)c5cc(Cl)cc(C#N)c5)cc4)n3)CC2)CC1]

3-chloro-5-(2-(4-((2-chloropyrimidin-4-yl)methoxy)phenyl)propan-2-yl)benzonitrile (9d) (400 mg, 1.00 mmol) was dissolved in 20 mL of DMF, and tert-butyl 4-(piperazin-1-yl)piperidine-1-carboxylate (34a) (see WO 2013163262 for the synthetic method) (325 mg, 1.20 mmol) and potassium bicarbonate (201 mg, 2.00 mmol) were successively added. The mixture was warmed to 60° C. and reacted for 3 h. The reaction liquid was cooled to room temperature, and 50 mL of water was slowly added. The mixture was extracted with ethyl acetate (50 mL×3), and the organic phase was washed with water (50 mL×2), dried over anhydrous sodium sulfate, and concentrated under reduced pressure. The crude product was separated and purified by silica gel column chromatography (dichloromethane/methanol (v/v)=20:1), to afford 34b (540 mg, yield from compound 9d: 86%).

LCMS m/z=631.3 [M+1]⁺

### Step 2: 3-chloro-5-(2-(4-((2-(4-(piperidin-4-yl)piperazin-1-yl)pyrimidin-4-yl)methoxy)phenyl)propan-2-yl)benzonitrile (34c) trifluoroacetate

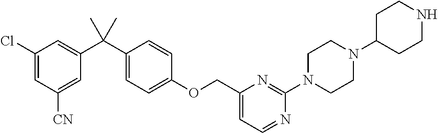 [SMILES: CC(C)(c1ccc(OCc2ccnc(N3CCN(C4CCNCC4)CC3)n2)cc1)c1cc(Cl)cc(C#N)c1]

34b (60 mg, 0.095 mmol) was dissolved in 10 mL of DCM, and 4 mL of trifluoroacetic acid was added. The mixture was stirred at room temperature for 2 h. The reaction liquid was concentrated under reduced pressure, to afford crude 34c trifluoroacetate (75 mg).

LCMS m/z=531.3 [M+1]⁺

### Step 3: tert-butyl 2-(4-(4-(4-((4-(2-(3-chloro-5-cyanophenyl)propan-2-yl)phenoxy)methyl)pyrimidin-2-yl)piperazin-1-yl)piperidin-1-yl)-7-azaspiro[3.5]nonane-7-carboxylate (34d)

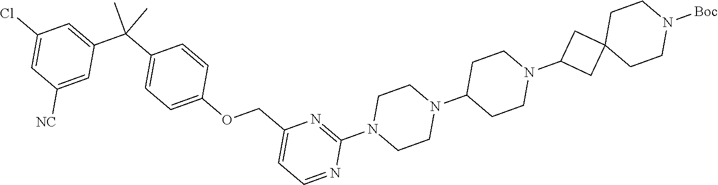 [SMILES: CC(C)(C)OC(=O)N1CCC2(CC1)CC(N1CCC(N3CCN(c4nccc(COc5ccc(C(C)(C)c6cc(Cl)cc(C#N)c6)cc5)n4)CC3)CC1)C2]

The above crude 34c trifluoroacetate (70 mg) was dissolved in 15 mL of DCE, and solid sodium bicarbonate (32 mg, 0.38 mmol) was added. The mixture was stirred at room temperature for 20 min, and then tert-butyl 2-oxo-7-azaspiro[3.5]nonane-7-carboxylate (45 mg, 0.19 mmol) and 0.05 mL of acetic acid were successively added. The mixture was stirred at room temperature for 60 min, and then sodium triacetoxyborohydride (41 mg, 0.19 mmol) was added. The resulting mixture was reacted at room temperature for 16 h. To the reaction system was slowly added 20 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=8:1), to afford 34d (55 mg, two-step yield from compound 34b: 82%).

LCMS m/z=754.4 [M+1]⁺

### Step 4: 3-(2-(4-((2-(4-(1-(7-azaspiro[3.5]nonan-2-yl)piperidin-4-yl)piperazin-1-yl)pyrimidin-4-yl)methoxy)phenyl)propan-2-yl)-5-chlorobenzonitrile (34e) trifluoroacetate

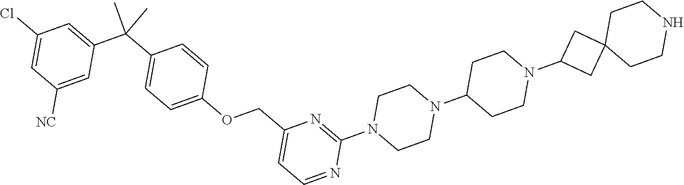 [SMILES: CC(C)(c1ccc(OCc2ccnc(N3CCN(C4CCN(C5CC6(CCNCC6)C5)CC4)CC3)n2)cc1)c1cc(Cl)cc(C#N)c1]

34d (55 mg, 0.073 mmol) was dissolved in 5 mL of DCM, and 2 mL of trifluoroacetic acid was added. The mixture was stirred at room temperature for 2 h. The reaction liquid was concentrated under reduced pressure, to afford crude 34e trifluoroacetate (70 mg).

LCMS m/z=654.4 [M+1]⁺

### Step 5: 3-chloro-5-(2-(4-((2-(4-(1-(7-(2-(2,6-dioxopiperidin-3-yl)-1,3-dioxoisoindolin-5-yl)-7-azaspiro[3.5]nonan-2-yl)piperidin-4-yl)piperazin-1-yl)pyrimidin-4-yl)methoxy)phenyl)propan-2-yl)benzonitrile (Compound 34) trifluoroacetate

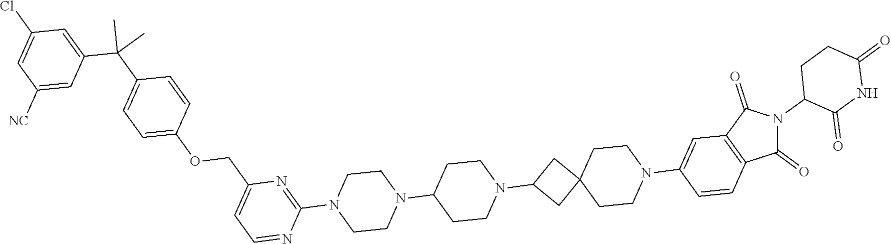 [SMILES: CC(C)(c1ccc(OCc2ccnc(N3CCN(C4CCN(C5CC6(CCN(c7ccc8c(c7)C(=O)N(C7CCC(=O)NC7=O)C8=O)CC6)C5)CC4)CC3)n2)cc1)c1cc(Cl)cc(C#N)c1]

The above crude 34e trifluoroacetate (70 mg) was dissolved in 4 mL of DMSO, and solid sodium bicarbonate (30 mg, 0.36 mmol) was added. The mixture was stirred at room temperature for 10 min, and 0.2 mL of DIPEA and 2-(2,6-dioxopiperidin-3-yl)-5-fluoroisoindoline-1,3-dione (see WO 2017197056 for the synthetic method) (40 mg, 0.14 mmol) were added. The reaction was stirred at 80° C. for 5 h. The reaction liquid was cooled to room temperature, and 50 mL of water was added. The mixture was filtered, and the filter cake was washed with 10 mL of water, dissolved with 50 mL of dichloromethane, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 34 trifluoroacetate (10 mg).

¹H NMR (400 MHz, DMSO-d₆) δ 11.05 (s, 1H), 8.46 (d, 1H), 7.87-7.83 (m, 1H), 7.70-7.62 (m, 2H), 7.56-7.50 (m, 1H), 7.37-7.32 (m, 1H), 7.30-7.23 (m, 1H), 7.21-7.14 (m, 2H), 7.00-6.91 (m, 2H), 6.86 (d, 1H), 5.12-4.98 (m, 3H), 4.60-4.30 (m, 4H), 3.78-3.65 (m, 1H), 3.65-3.53 (m, 2H), 3.53-3.45 (m, 2H), 3.45-3.37 (m, 2H), 3.35-2.96 (m, 4H), 2.96-2.72 (m, 3H), 2.65-2.52 (m, 3H), 2.45-2.30 (m, 2H), 2.30-2.15 (m, 2H), 2.12-1.96 (m, 3H), 1.96-1.80 (m, 2H), 1.76-1.52 (m, 10H).

LCMS m/z=910.3 [M+1]⁺.

### Example 35

### 3-chloro-2-(2-chloroethoxy)-5-(2-(4-((2-(9-((1-(2-(2,6-dioxopiperidin-3-yl)-1,3-dioxoisoindolin-5-yl)piperidin-4-yl)methyl)-3,9-diazaspiro[5.5]undecan-3-yl)pyrimidin-4-yl)methoxy)phenyl)propan-2-yl)benzonitrile (Compound 35)

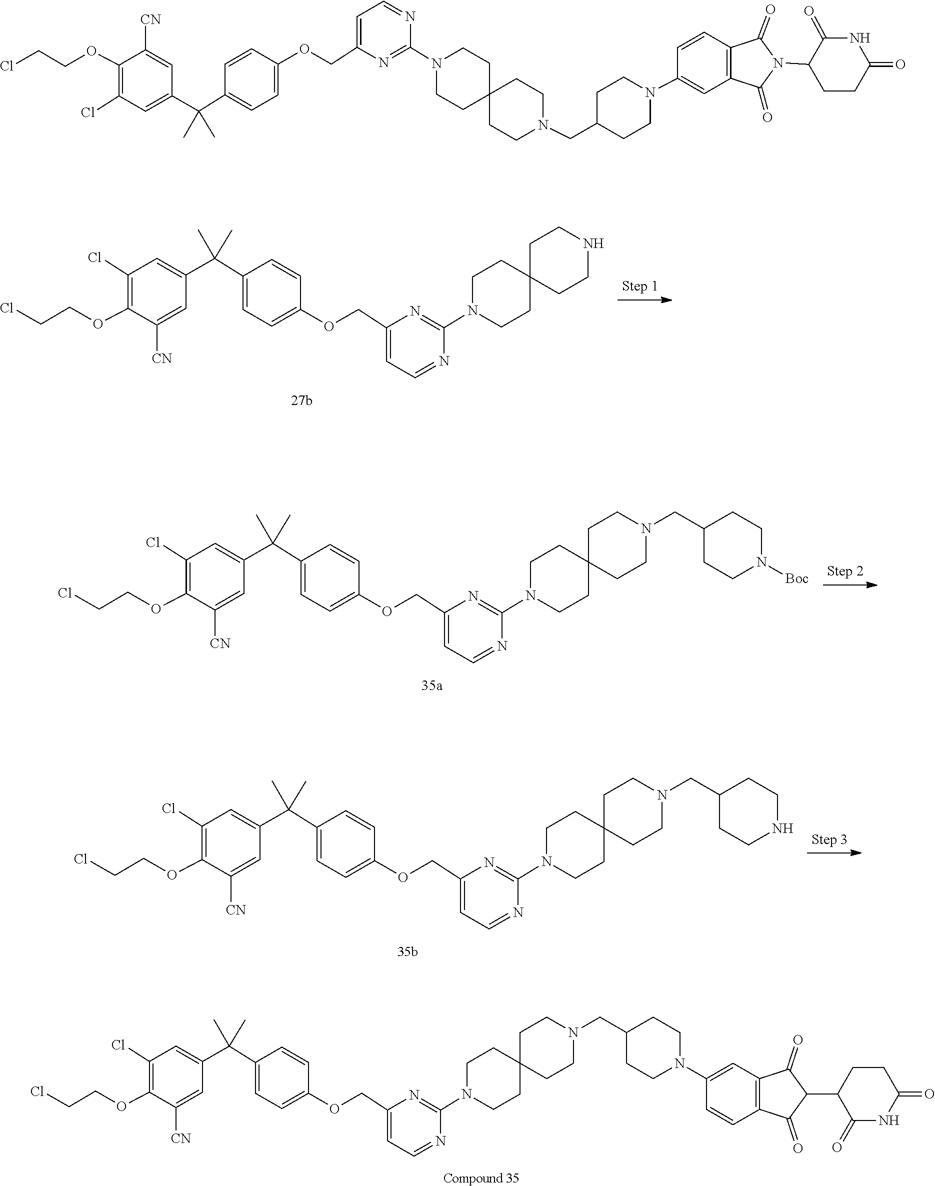 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1 ; CC(C)(c1ccc(OCc2ccnc(N3CCC4(CCNCC4)CC3)n2)cc1)c1cc(Cl)c(OCCCl)c(C#N)c1 ; CC(C)(C)OC(=O)N1CCC(CN2CCC3(CC2)CCN(c2nccc(COc4ccc(C(C)(C)c5cc(Cl)c(OCCCl)c(C#N)c5)cc4)n2)CC3)CC1 ; CC(C)(c1ccc(OCc2ccnc(N3CCC4(CCN(CC5CCNCC5)CC4)CC3)n2)cc1)c1cc(Cl)c(OCCCl)c(C#N)c1 ; CC(C)(c1ccc(OCc2ccnc(N3CCC4(CCN(CC5CCN(c6ccc7c(c6)C(=O)C(C6CCC(=O)NC6=O)C7=O)CC5)CC4)CC3)n2)cc1)c1cc(Cl)c(OCCCl)c(C#N)c1]

### Step 1: tert-butyl 4-((9-(4-((4-(2-(3-chloro-4-(2-chloroethoxy)-5-cyanophenyl)propan-2-yl)phenoxy)methyl)pyrimidin-2-yl)-3,9-diazaspiro[5.5]undecan-3-yl)methyl)piperidine-1-carboxylate (35a)

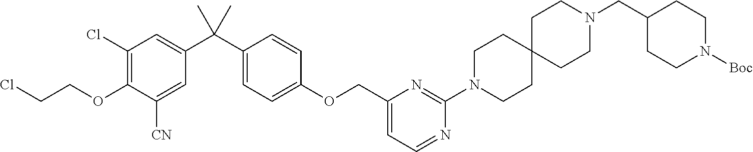 [SMILES: CC(C)(C)OC(=O)N1CCC(CN2CCC3(CC2)CCN(c2nccc(COc4ccc(C(C)(C)c5cc(Cl)c(OCCCl)c(C#N)c5)cc4)n2)CC3)CC1]

The above crude 27b trifluoroacetate (70 mg) was dissolved in 15 mL of DCE, and solid sodium bicarbonate (15 mg, 0.18 mmol) was added. The mixture was stirred at room temperature for 20 min, and then tert-butyl 4-formylpiperidine-1-carboxylate (30 mg, 0.14 mmol) and 0.05 mL of acetic acid were successively added. The mixture was stirred at room temperature for 60 min, and then sodium triacetoxyborohydride (30 mg, 0.14 mmol) was added. The resulting mixture was reacted at room temperature for 16 h. To the reaction system was slowly added 30 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 35a (50 mg, two-step yield from compound 27a: 73%).

LCMS m/z=791.3 [M+1]⁺

### Step 2: 3-chloro-2-(2-chloroethoxy)-5-(2-(4-((2-(9-(piperidin-4-ylmethyl)-3,9-diazaspiro[5.5]undecan-3-yl)pyrimidin-4-yl)methoxy)phenyl)propan-2-yl)benzonitrile (35b) trifluoroacetate

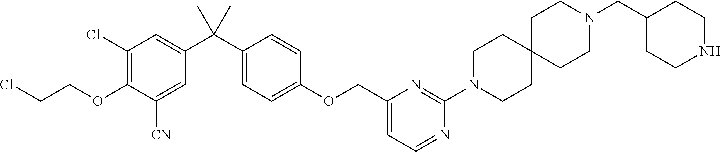 [SMILES: CC(C)(c1ccc(OCc2ccnc(N3CCC4(CCN(CC5CCNCC5)CC4)CC3)n2)cc1)c1cc(Cl)c(OCCCl)c(C#N)c1]

35a (50 mg, 0.06 mmol) was dissolved in 5 mL of DCM, and 2 mL of trifluoroacetic acid was added. The mixture was stirred at room temperature for 2 h. The reaction liquid was concentrated under reduced pressure, to afford crude 35b trifluoroacetate (60 mg).

LC-MS m/z=691.3 [M+1]⁺

### Step 3: 3-chloro-2-(2-chloroethoxy)-5-(2-(4-((2-(9-((1-(2-(2,6-dioxopiperidin-3-yl)-1,3-dioxoisoindolin-5-yl)piperidin-4-yl)methyl)-3,9-diazaspiro[5.5]undecan-3-yl)pyrimidin-4-yl)methoxy)phenyl)propan-2-yl)benzonitrile (Compound 35)

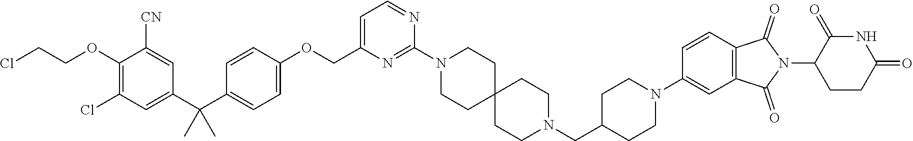 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)c(OCCCl)c(C#N)c1]

The above crude 35b trifluoroacetate (60 mg) was dissolved in 4 mL of DMSO, and solid sodium bicarbonate (25 mg, 0.30 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (34 mg, 0.12 mmol) were added. The resulting mixture was warmed to 80° C. and reacted for 5 h. The reaction liquid was cooled to room temperature, and 20 mL of water was added. The mixture was filtered, and the filter cake was washed with 10 mL of water, dissolved with 50 mL of dichloromethane, dried over anhydrous sodium sulfate, and concentrated under reduced pressure. The crude product was then separated and purified by a preparative plate (dichloromethane/methanol (v/v)=12:1), to afford compound 35 (25 mg, two-step yield from compound 35a: 44%).

¹H NMR (400 MHz, CDCl₃) δ 8.30 (d, 1H), 8.09 (br.s, 1H), 7.67 (d, 1H), 7.44 (d, 1H), 7.32 (d, 1H), 7.28 (d, 1H), 7.13-6.99 (m, 3H), 6.93-6.84 (m, 2H), 6.67 (d, 1H), 4.99-4.88 (m, 3H), 4.42 (t, 2H), 4.03-3.70 (m, 8H), 3.08-2.92 (m, 2H), 2.92-2.66 (m, 3H), 2.60-2.30 (m, 4H), 2.30-2.06 (m, 3H), 2.00-1.83 (m, 2H), 1.74-1.44 (m, 15H), 1.40-1.17 (m, 2H).

LCMS m/z=947.5 [M+1]⁺.

### Example 36

### 3-chloro-2-(2-chloroethoxy)-5-(2-(4-((2-(4-(1-((1-((1-(2,6-dioxopiperidin-3-yl)-3-methyl-2-oxo-2,3-dihydro-1H-benzo[d]imidazol-4-yl)methyl)piperidin-4-yl)methyl)piperidin-4-yl)piperazin-1-yl)pyrimidin-4-yl)methoxy)phenyl)propan-2-yl)benzonitrile (Compound 36) trifluoroacetate

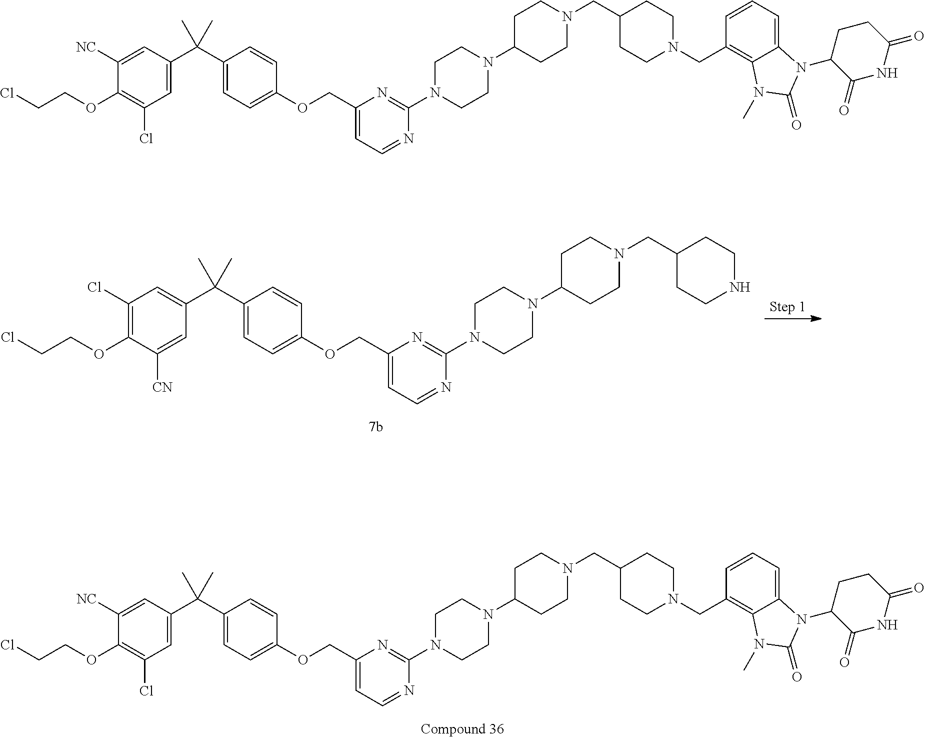 [SMILES: Cn1c(=O)n(C2CCC(=O)NC2=O)c2cccc(CN3CCC(CN4CCC(N5CCN(c6nccc(COc7ccc(C(C)(C)c8cc(Cl)c(OCCCl)c(C#N)c8)cc7)n6)CC5)CC4)CC3)c21 ; CC(C)(c1ccc(OCc2ccnc(N3CCN(C4CCN(CC5CCNCC5)CC4)CC3)n2)cc1)c1cc(Cl)c(OCCCl)c(C#N)c1 ; Cn1c(=O)n(C2CCC(=O)NC2=O)c2cccc(CN3CCC(CN4CCC(N5CCN(c6nccc(COc7ccc(C(C)(C)c8cc(Cl)c(OCCCl)c(C#N)c8)cc7)n6)CC5)CC4)CC3)c21]

The above crude 7b trifluoroacetate (50 mg) and 1-(2,6-dioxopiperidin-3-yl)-3-methyl-2-oxo-2,3-dihydro-1H-benzo[d]imidazole-4-carbaldehyde (see WO 2020113233 for the synthetic method) (20 mg, 0.07 mmol) were dissolved in 4 mL of DMAc, and 0.02 mL of acetic acid was added. The mixture was stirred at room temperature for 2 h, and sodium triacetoxyborohydride (75 mg, 0.35 mmol) was added. The resulting mixture was stirred at room temperature for 12 h. To the reaction liquid was added 30 mL of saturated aqueous sodium bicarbonate solution, and the mixture was extracted with ethyl acetate (50 mL×3), dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 36 trifluoroacetate (10 mg).

¹H NMR (400 MHz, DMSO-d₆) δ 11.12 (s, 1H), 8.34 (d, 1H), 7.68-7.52 (m, 2H), 7.35-7.05 (m, 5H), 6.94 (d, 2H), 6.66 (d, 1H), 5.50-5.40 (m, 1H), 4.97 (s, 2H), 4.80-4.66 (m, 2H), 4.66-4.55 (m, 2H), 4.46-4.38 (m, 2H), 4.03-3.90 (m, 2H), 3.70-3.42 (m, 6H), 3.37-3.02 (m, 2H), 3.02-2.56 (m, 7H), 2.54-2.47 (m, 3H), 2.21-1.81 (m, 5H), 1.80-1.68 (m, 2H), 1.63 (s, 6H), 1.57-1.28 (m, 6H), 1.19-1.00 (m, 2H).

LCMS m/z=488.8 [M/2+1]+

LCMS m/z=488.8 [M/2+1]⁺

### Example 37

### 3-chloro-5-(2-(4-((2-(4-((1′-(2-(2,6-dioxopiperidin-3-yl)-1,3-dioxoisoindolin-5-yl)-[4,4′-bipiperidin]-1-yl)methyl)piperidin-1-yl)pyrimidin-4-yl)methoxy)phenyl)propan-2-yl)benzonitrile (Compound 37) trifluoroacetate

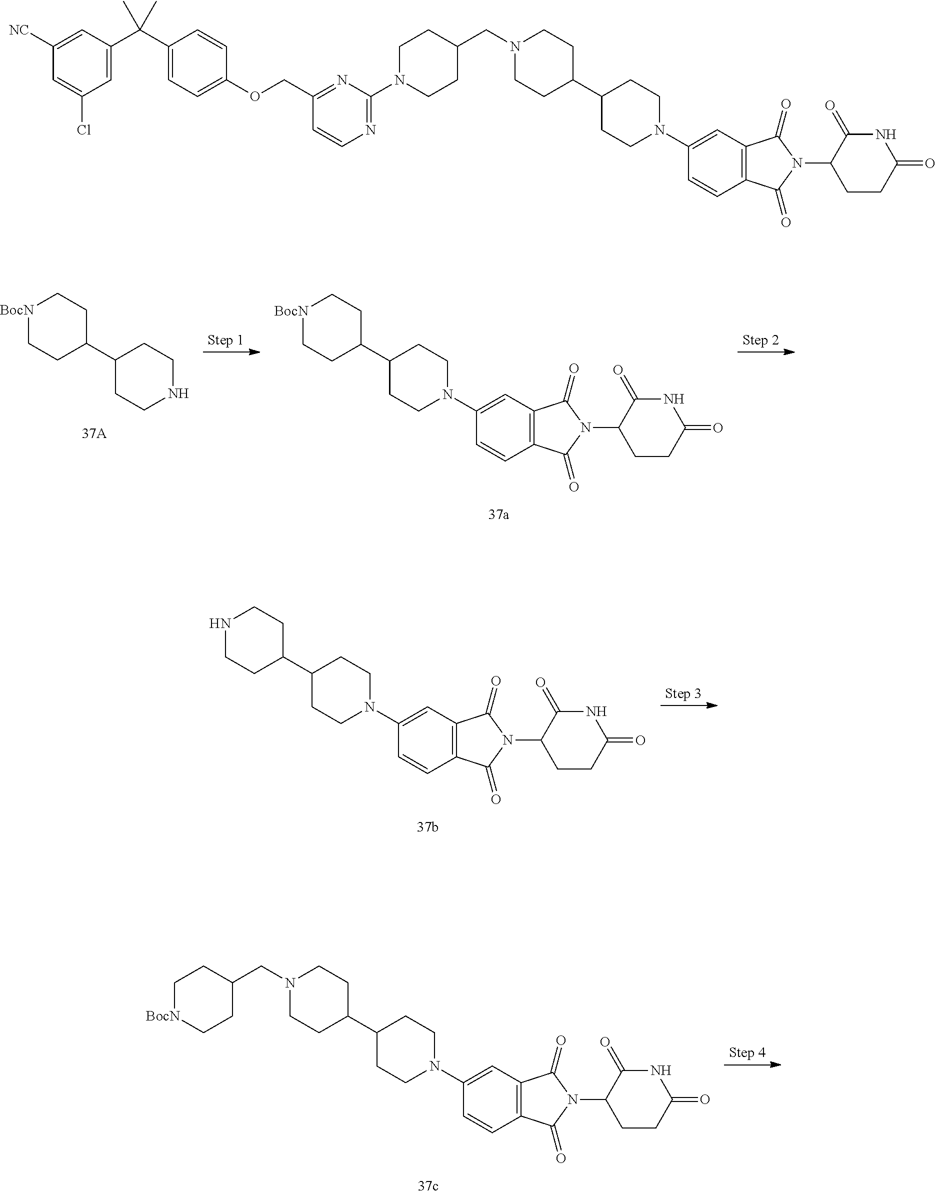 [SMILES: CC(C)(c1ccc(OCc2ccnc(N3CCC(CN4CCC(C5CCN(c6ccc7c(c6)C(=O)N(C6CCC(=O)NC6=O)C7=O)CC5)CC4)CC3)n2)cc1)c1cc(Cl)cc(C#N)c1 ; CC(C)(C)OC(=O)N1CCC(C2CCNCC2)CC1 ; CC(C)(C)OC(=O)N1CCC(C2CCN(c3ccc4c(c3)C(=O)N(C3CCC(=O)NC3=O)C4=O)CC2)CC1 ; O=C1CCC(N2C(=O)c3ccc(N4CCC(C5CCNCC5)CC4)cc3C2=O)C(=O)N1 ; CC(C)(C)OC(=O)N1CCC(CN2CCC(C3CCN(c4ccc5c(c4)C(=O)N(C4CCC(=O)NC4=O)C5=O)CC3)CC2)CC1]

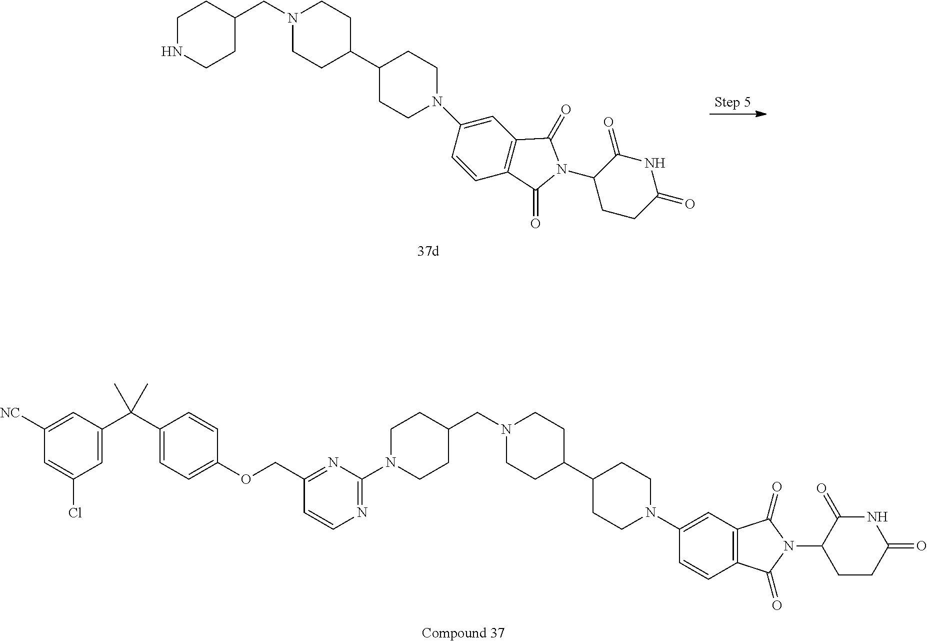 [SMILES: O=C1CCC(N2C(=O)c3ccc(N4CCC(C5CCN(CC6CCNCC6)CC5)CC4)cc3C2=O)C(=O)N1 ; CC(C)(c1ccc(OCc2ccnc(N3CCC(CN4CCC(C5CCN(c6ccc7c(c6)C(=O)N(C6CCC(=O)NC6=O)C7=O)CC5)CC4)CC3)n2)cc1)c1cc(Cl)cc(C#N)c1]

### Step 1: tert-butyl 1′-(2-(2,6-dioxopiperidin-3-yl)-1,3-dioxoisoindolin-5-yl)-[4,4′-bipiperidine]-1-carboxylate (37a)

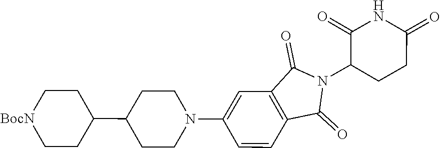 [SMILES: CC(C)(C)OC(=O)N1CCC(C2CCN(c3ccc4c(c3)C(=O)N(C3CCC(=O)NC3=O)C4=O)CC2)CC1]

2-(2,6-dioxopiperidin-3-yl)-5-fluoroisoindoline-1,3-dione (see WO 2017197056 for the synthetic method) (300 mg, 1.09 mmol) was dissolved in 10 mL of DMF, and tert-butyl [4,4′-bipiperidine]-1-carboxylate (37A) (290 mg, 1.08 mmol) and solid potassium bicarbonate (266 mg, 2.66 mmol) were successively added. The mixture was warmed to 90° C. and stirred for 4 h. The mixture was cooled to room temperature, and 30 mL of water was added. The resulting mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 37a (500 mg, yield: 87%).

### Step 2: 5-([4,4′-bipiperidin]-1-yl)-2-(2,6-dioxopiperidin-3-yl)isoindoline-1,3-dione (37b)

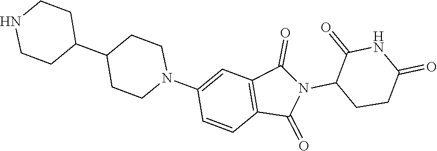 [SMILES: O=C1CCC(N2C(=O)c3ccc(N4CCC(C5CCNCC5)CC4)cc3C2=O)C(=O)N1]

37a (500 mg, 0.95 mmol) was dissolved in 5 mL of dichloromethane, and 2 mL of trifluoroacetic acid was added. The mixture was stirred at room temperature for 4 h. The reaction system was concentrated under reduced pressure, and 50 mL of dichloromethane was added. The mixture was adjusted to pH 10 with a saturated sodium bicarbonate solution, extracted with 50 mL of dichloromethane, dried over anhydrous sodium sulfate, and concentrated under reduced pressure, to afford crude 37b (300 mg).

Step 3: tert-butyl 4-((1′-(2-(2,6-dioxopiperidin-3-yl)-1,3-dioxoisoindolin-5-yl)-[4,4′-bipiperidin]-1-yl)methyl)piperidine-1-carboxylate (37c)

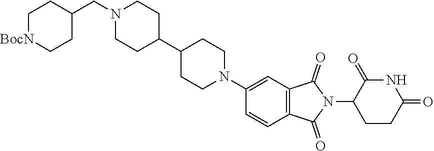 [SMILES: CC(C)(C)OC(=O)N1CCC(CN2CCC(C3CCN(c4ccc5c(c4)C(=O)N(C4CCC(=O)NC4=O)C5=O)CC3)CC2)CC1]

The above crude 37b (250 mg) was dissolved in 15 mL of 1,2-dichloroethane, and tert-butyl 4-formylpiperidine-1-carboxylate (200 mg, 0.94 mmol) was added. The mixture was stirred for 1 h, and then sodium triacetoxyborohydride (300 mg, 1.42 mmol) was added. The resulting mixture was reacted at room temperature for 16 h. To the reaction liquid was slowly added 20 mL of saturated sodium bicarbonate solution, and the mixture was extracted twice with 60 mL of ethyl acetate. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 37c (300 mg, two-step yield from compound 37a: 61%).

LCMS m/z=622.3 [M+1]⁺

### Step 4: 2-(2,6-dioxopiperidin-3-yl)-5-(1′-(piperidin-4-ylmethyl)-[4,4′-bipiperidin]-1-yl)isoindoline-1,3-dione (37d)

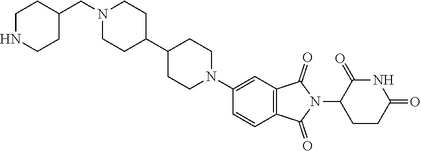 [SMILES: O=C1CCC(N2C(=O)c3ccc(N4CCC(C5CCN(CC6CCNCC6)CC5)CC4)cc3C2=O)C(=O)N1]

37c (300 mg, 0.48 mmol) was dissolved in 2 mL of DCM, and 1 mL of trifluoroacetic acid was added. The mixture was stirred at room temperature for 4 h. The reaction system was concentrated under reduced pressure, dissolved with 50 mL of DCM, adjusted to pH 10 with a saturated sodium bicarbonate solution, extracted with 50 mL of dichloromethane, dried over anhydrous sodium sulfate, and concentrated under reduced pressure, to afford crude 37d (200 mg).

LCMS m/z=522.2 [M+1]⁺

### Step 5: 3-chloro-5-(2-(4-((2-(4-((1′-(2-(2,6-dioxopiperidin-3-yl)-1,3-dioxoisoindolin-5-yl)-[4,4′-bipiperidin]-1-yl)methyl)piperidin-1-yl)pyrimidin-4-yl)methoxy)phenyl)propan-2-yl)benzonitrile (Compound 37) trifluoroacetate

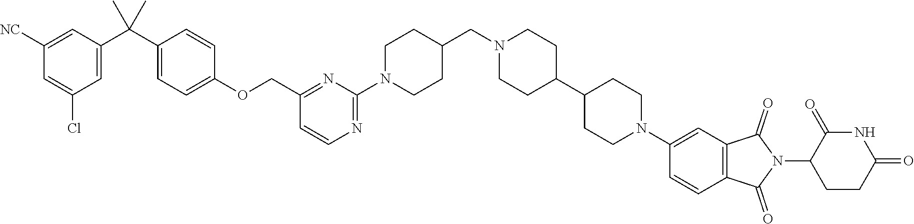 [SMILES: CC(C)(c1ccc(OCc2ccnc(N3CCC(CN4CCC(C5CCN(c6ccc7c(c6)C(=O)N(C6CCC(=O)NC6=O)C7=O)CC5)CC4)CC3)n2)cc1)c1cc(Cl)cc(C#N)c1]

The above crude 37d (200 mg) was dissolved in 15 mL of DMF, and 3-chloro-5-(2-(4-((2-chloropyrimidin-4-yl)methoxy)phenyl)propan-2-yl)benzonitrile (9d) (150 mg, 0.38 mmol) and solid potassium bicarbonate (110 mg, 1.10 mmol) were successively added. The mixture was warmed to 90° C. and stirred for 4 h. The mixture was cooled to room temperature, and 30 mL of water was added. The mixture was extracted twice with 30 mL of ethyl acetate, and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 37 trifluoroacetate (75 mg).

¹H NMR (400 MHz, CD₃OD) δ 8.29 (d, 1H), 7.66 (d, 1H), 7.62-7.56 (m, 1H), 7.54-7.45 (m, 2H), 7.34 (d, 1H), 7.26-7.10 (m, 3H), 7.00-6.87 (m, 2H), 6.77 (d, 1H), 5.13-4.95 (m, 3H), 4.82-4.73 (m, 2H), 4.17-4.00 (m, 2H), 3.75-3.59 (m, 2H), 3.10-2.62 (m, 11H), 2.29-1.99 (m, 4H), 1.99-1.78 (m, 4H), 1.72-1.20 (m, 14H).

LCMS m/z=442.3 [M/2+1]⁺

### Example 38: 3-chloro-5-(2-(4-((2-(1′-((1-(2-(2,6-dioxopiperidin-3-yl)-1,3-dioxoisoindolin-5-yl)piperidin-4-yl)methyl)-[4,4′-bipiperidin]-1-yl)pyrimidin-4-yl)methoxy)phenyl)propan-2-yl)benzonitrile (compound 38) trifluoroacetate

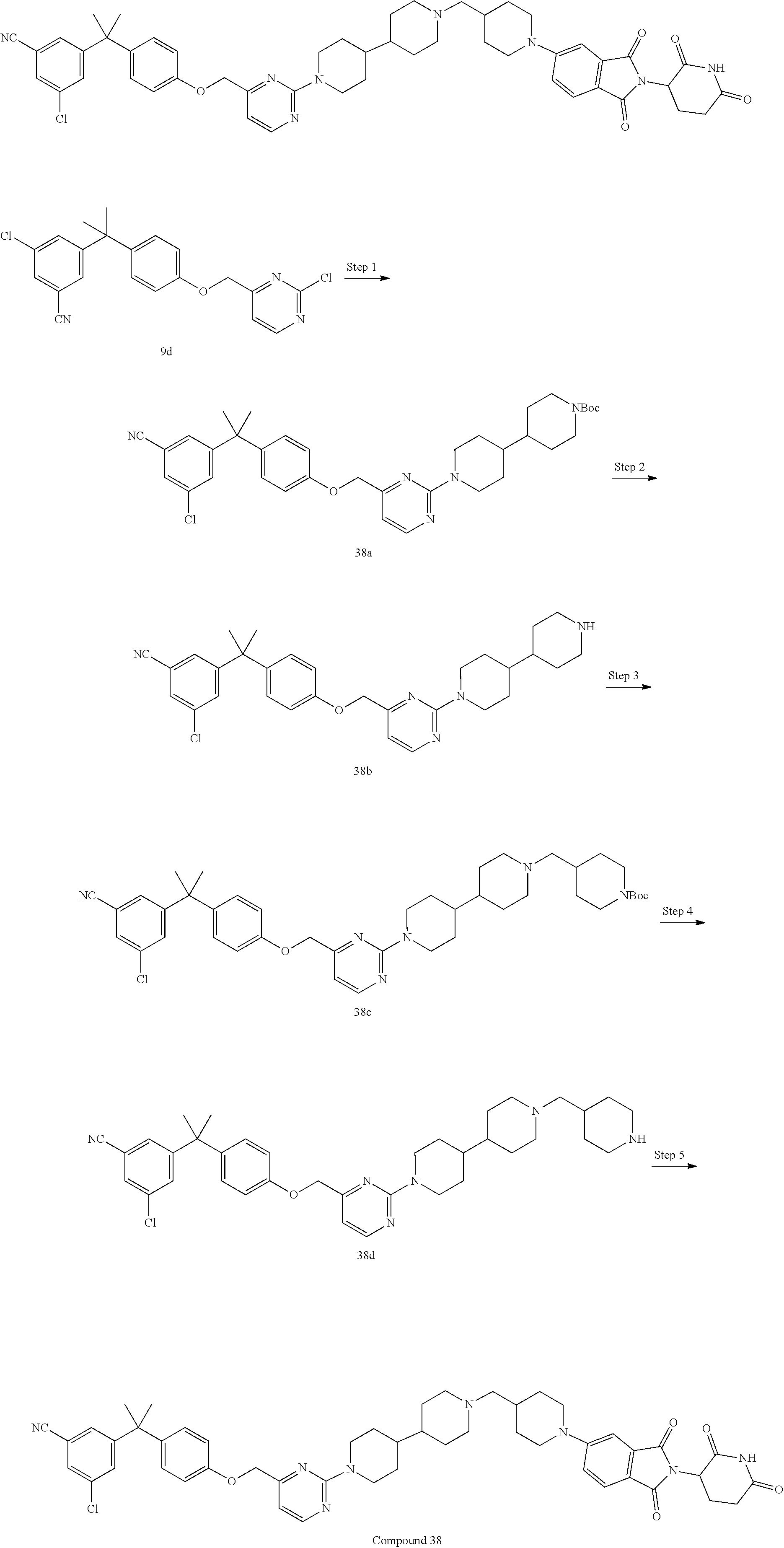

### Step 1: tert-butyl 1′-(4-((4-(2-(3-chloro-5-cyanophenyl)propan-2-yl)phenoxy)methyl)pyrimidin-2-yl)-[4,4′-bipiperidine]-1-carboxylate (38a)

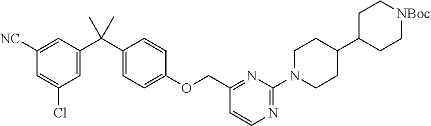 [SMILES: CC(C)(C)OC(=O)N1CCC(C2CCN(c3nccc(COc4ccc(C(C)(C)c5cc(Cl)cc(C#N)c5)cc4)n3)CC2)CC1]

3-chloro-5-(2-(4-((2-chloropyrimidin-4-yl)methoxy)phenyl)propan-2-yl)benzonitrile (9d) (600 mg, 1.51 mmol) was dissolved in 20 mL of DMF, and tert-butyl [4,4′-bipiperidine]-1-carboxylate (490 mg, 1.82 mmol) and solid potassium bicarbonate (300 mg, 3.00 mmol) were successively added. The mixture was warmed to 90° C. and stirred for 4 h. The mixture was cooled to room temperature, and 50 mL of water was added. The resulting mixture was extracted twice with 50 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 38a (800 mg, yield: 84%).

LCMS m/z=630.2 [M+1]⁺

### Step 2: 3-(2-(4-((2-([4,4′-bipiperidin]-1-yl)pyrimidin-4-yl)methoxy)phenyl)propan-2-yl)-5-chlorobenzonitrile (38b)

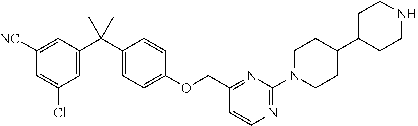 [SMILES: CC(C)(c1ccc(OCc2ccnc(N3CCC(C4CCNCC4)CC3)n2)cc1)c1cc(Cl)cc(C#N)c1]

38a (800 mg, 1.27 mmol) was dissolved in 15 mL of dichloromethane, and 5 mL of trifluoroacetic acid was added. The mixture was stirred at room temperature for 4 h. The reaction system was concentrated under reduced pressure, and 100 mL of dichloromethane was added. The mixture was adjusted to pH 9 with a saturated sodium bicarbonate solution, extracted with 100 mL of dichloromethane, dried over anhydrous sodium sulfate, and concentrated under reduced pressure, to afford crude 38b (600 mg).

LCMS m/z=530.3 [M+1]⁺

### Step 3: tert-butyl 4-((1′-(4-((4-(2-(3-chloro-5-cyanophenyl)propan-2-yl)phenoxy)methyl)pyrimidin-2-yl)-[4,4′-bipiperidin]-1-yl)methyl)piperidine-1-carboxylate (38c)

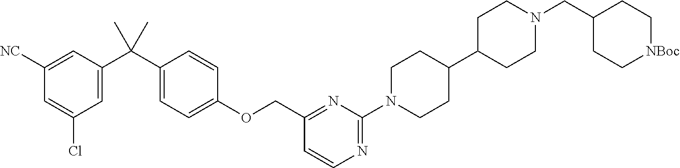 [SMILES: CC(C)(C)OC(=O)N1CCC(CN2CCC(C3CCN(c4nccc(COc5ccc(C(C)(C)c6cc(Cl)cc(C#N)c6)cc5)n4)CC3)CC2)CC1]

The above crude 38b (190 mg) was dissolved in 15 mL of 1,2-dichloroethane, and tert-butyl 4-formylpiperidine-1-carboxylate (150 mg, 0.70 mmol) was added. The mixture was stirred for 1 h, and then sodium triacetoxyborohydride (220 mg, 1.04 mmol) was added. The resulting mixture was reacted at room temperature for 16 h. To the reaction liquid was slowly added 50 mL of saturated sodium bicarbonate solution, and the mixture was extracted twice with 100 mL of ethyl acetate. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 38c (150 mg, two-step yield from compound 38a: 51%).

LCMS m/z=727.3 [M+1]⁺

### Step 4: 3-chloro-5-(2-(4-((2-(1′-(piperidin-4-ylmethyl)-[4,4′-bipiperidin]-1-yl)pyrimidin-4-yl)methoxy)phenyl)propan-2-yl)benzonitrile (38d) trifluoroacetate

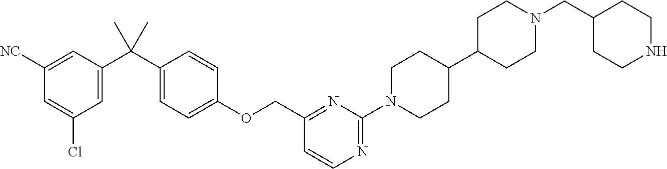 [SMILES: CC(C)(c1ccc(OCc2ccnc(N3CCC(C4CCN(CC5CCNCC5)CC4)CC3)n2)cc1)c1cc(Cl)cc(C#N)c1]

38c (0.15 g, 0.21 mmol) was dissolved in 2 mL of DCM, and 1 mL of trifluoroacetic acid was added.

The mixture was stirred at room temperature for 2 h. The reaction system was concentrated under reduced pressure, to afford crude 38d trifluoroacetate (155 mg).

LCMS m/z=627.2 [M+1]⁺

### Step 6: 3-chloro-5-(2-(4-((2-(1′-((1-(2-(2,6-dioxopiperidin-3-yl)-1,3-dioxoisoindolin-5-yl)piperidin-4-yl)methyl)-[4,4′-bipiperidin]-1-yl)pyrimidin-4-yl)methoxy)phenyl)propan-2-yl)benzonitrile (compound 38) trifluoroacetate

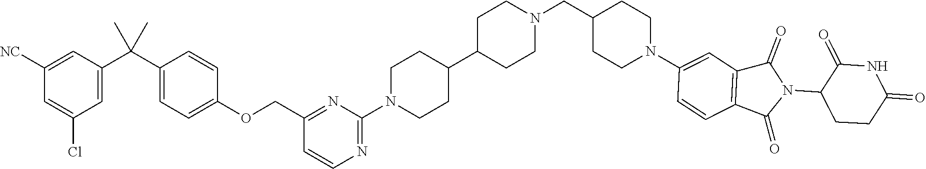 [SMILES: CC(C)(c1ccc(OCc2ccnc(N3CCC(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

The above crude 38d trifluoroacetate (155 mg) was dissolved in 5 mL of DMSO, and 1 mL of DIPEA and 2-(2,6-dioxopiperidin-3-yl)-5-fluoroisoindoline-1,3-dione (see WO 2017197056 for the synthetic method) (60 mg, 0.22 mmol) were added. The reaction was stirred at 80° C. for 8 h. The mixture was cooled to room temperature, and to the reaction system was slowly added 30 mL of saturated sodium bicarbonate solution. The mixture was extracted twice with 60 mL of ethyl acetate, and the organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 38 trifluoroacetate (15 mg).

¹H NMR (400 MHz, CDCl₃) δ 8.35-8.24 (m, 1H), 8.03 (br.s, 1H), 7.66 (d, 1H), 7.49-7.34 (m, 3H), 7.30-7.25 (m, 1H), 7.13-7.00 (m, 3H), 6.93-6.83 (m, 2H), 6.66 (d, 1H), 4.99-4.88 (m, 3H), 4.87-4.75 (m, 2H), 4.00-3.88 (m, 2H), 3.07-2.65 (m, 9H), 2.28-2.07 (m, 3H), 2.00-1.62 (m, 15H), 1.62-1.47 (m, 4H), 1.40-1.05 (m, 4H).

LCMS m/z=442.3 [M/2+1]⁺

### Example 39

### 3-chloro-5-(2-(4-((2-(4-((4-(2-(2,6-dioxopiperidin-3-yl)-1,3-dioxoisoindolin-4-yl)piperazin-1-yl)methyl)piperidin-1-yl)pyrimidin-4-yl)methoxy)phenyl)propan-2-yl)benzonitrile (Compound 39) trifluoroacetate

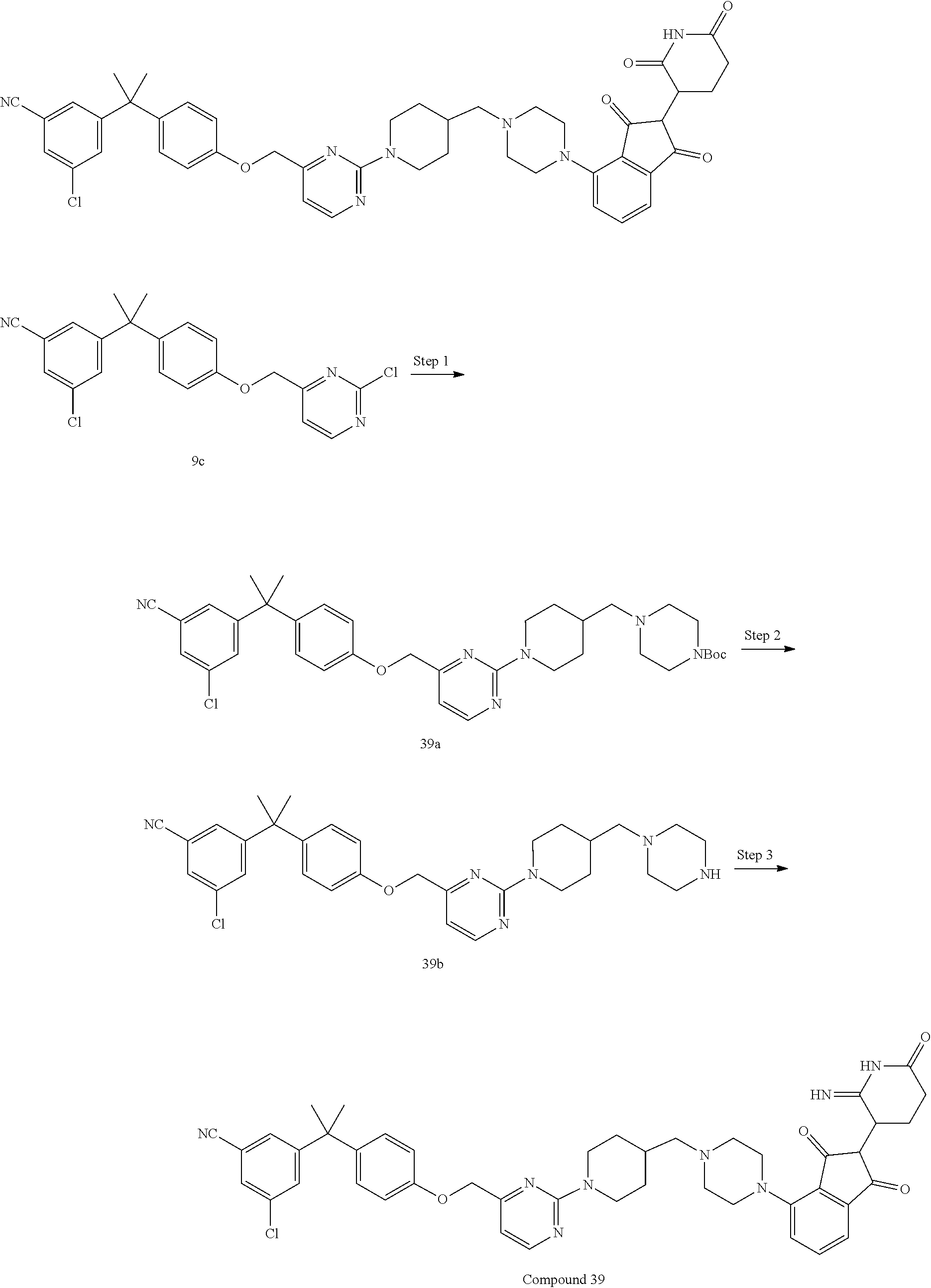 [SMILES: CC(C)(c1ccc(OCc2ccnc(N3CCC(CN4CCN(c5cccc6c5C(=O)C(C5CCC(=O)NC5=O)C6=O)CC4)CC3)n2)cc1)c1cc(Cl)cc(C#N)c1 ; CC(C)(c1ccc(OCc2ccnc(Cl)n2)cc1)c1cc(Cl)cc(C#N)c1 ; CC(C)(C)OC(=O)N1CCN(CC2CCN(c3nccc(COc4ccc(C(C)(C)c5cc(Cl)cc(C#N)c5)cc4)n3)CC2)CC1 ; CC(C)(c1ccc(OCc2ccnc(N3CCC(CN4CCNCC4)CC3)n2)cc1)c1cc(Cl)cc(C#N)c1 ; CC(C)(c1ccc(OCc2ccnc(N3CCC(CN4CCN(c5cccc6c5C(=O)C(C5CCC(=O)NC5=N)C6=O)CC4)CC3)n2)cc1)c1cc(Cl)cc(C#N)c1]

### Step 1: tert-butyl 4-((1-(4-((4-(2-(3-chloro-5-cyanophenyl)propan-2-yl)phenoxy)methyl)pyrimidin-2-yl)piperidin-4-yl)methyl)piperazine-1-carboxylate (39a)

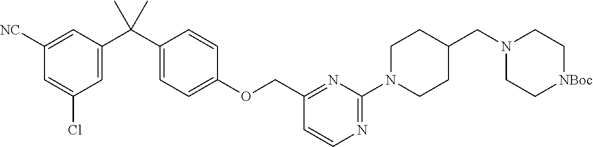 [SMILES: CC(C)(C)OC(=O)N1CCN(CC2CCN(c3nccc(COc4ccc(C(C)(C)c5cc(Cl)cc(C#N)c5)cc4)n3)CC2)CC1]

9d (200 mg, 0.5 mmol) was dissolved in 5 mL of DMF, and tert-butyl 4-(piperidin-4-ylmethyl)piperazine-1-carboxylate (see WO 2020201080 for the synthetic method) (200 mg, 0.7 mmol) and solid potassium bicarbonate (138 mg, 1.38 mmol) were successively added. The mixture was warmed to 70° C. and reacted for 2 h. The mixture was cooled to room temperature, and 30 mL of water was added. The resulting mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 39a (250 mg, yield: 78%).

LCMS m/z=645.3 [M+1]⁺

### Step 2: 3-chloro-5-(2-(4-((2-(4-(piperazin-1-ylmethyl)piperidin-1-yl)pyrimidin-4-yl)methoxy)phenyl)propan-2-yl)benzonitrile (39b)

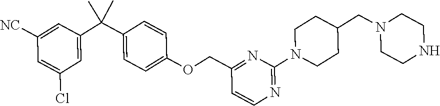 [SMILES: CC(C)(c1ccc(OCc2ccnc(N3CCC(CN4CCNCC4)CC3)n2)cc1)c1cc(Cl)cc(C#N)c1]

39a (250 mg, 0.39 mmol) was dissolved in 8 mL of dichloromethane, and 2 mL of trifluoroacetic acid was added. The mixture was stirred at room temperature for 2 h. The reaction system was concentrated under reduced pressure, and 30 mL of dichloromethane was added. The mixture was adjusted to pH 9 with a saturated sodium bicarbonate solution, extracted with 30 mL of dichloromethane, dried over anhydrous sodium sulfate, and concentrated under reduced pressure, to afford crude 39b (180 mg).

LCMS m/z=545.3 [M+1]⁺

### Step 3: 3-chloro-5-(2-(4-((2-(4-((4-(2-(2,6-dioxopiperidin-3-yl)-1,3-dioxoisoindolin-4-yl)piperazin-1-yl)methyl)piperidin-1-yl)pyrimidin-4-yl)methoxy)phenyl)propan-2-yl)benzonitrile (Compound 39) trifluoroacetate

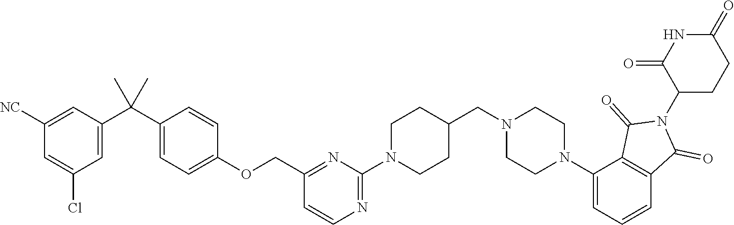 [SMILES: CC(C)(c1ccc(OCc2ccnc(N3CCC(CN4CCN(c5cccc6c5C(=O)N(C5CCC(=O)NC5=O)C6=O)CC4)CC3)n2)cc1)c1cc(Cl)cc(C#N)c1]

The above crude 39b (90 mg) was dissolved in 5 mL of DMSO, and 0.1 mL of DIPEA and 2-(2,6-dioxopiperidin-3-yl)-4-fluoroisoindoline-1,3-dione (see WO 2017197056 for the synthetic method) (0.1 g, 0.36 mmol) were added. The mixture was warmed to 85° C. and reacted for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 39 trifluoroacetate (19 mg).

¹H NMR (400 MHz, DMSO-d₆) δ 11.09 (s, 1H), 8.37 (d, 1H), 7.88-7.74 (m, 2H), 7.68-7.62 (m, 1H), 7.57-7.39 (m, 3H), 7.23-7.12 (m, 2H), 7.00-6.90 (m, 2H), 6.70 (d, 1H), 5.12 (dd, 1H), 4.98 (s, 2H), 4.75-4.58 (m, 2H), 4.02-3.76 (m, 4H), 3.47-3.09 (m, 6H), 3.03-2.82 (m, 3H), 2.69-2.54 (m, 2H), 2.28-2.12 (m, 1H), 2.11-1.99 (m, 1H), 1.92-1.78 (m, 2H), 1.64 (s, 6H), 1.28-1.10 (m, 2H).

LCMS m/z=801.3 [M+1]⁺

### Example 40

### 3-chloro-5-(2-(4-((2-(4-((4-(2-(2,6-dioxopiperidin-3-yl)-1,3-dioxoisoindolin-4-yl)piperazin-1-yl)methyl)-[1,4′-bipiperidin]-1′-yl)pyrimidin-4-yl)methoxy)phenyl)propan-2-yl)benzonitrile (Compound 40) trifluoroacetate

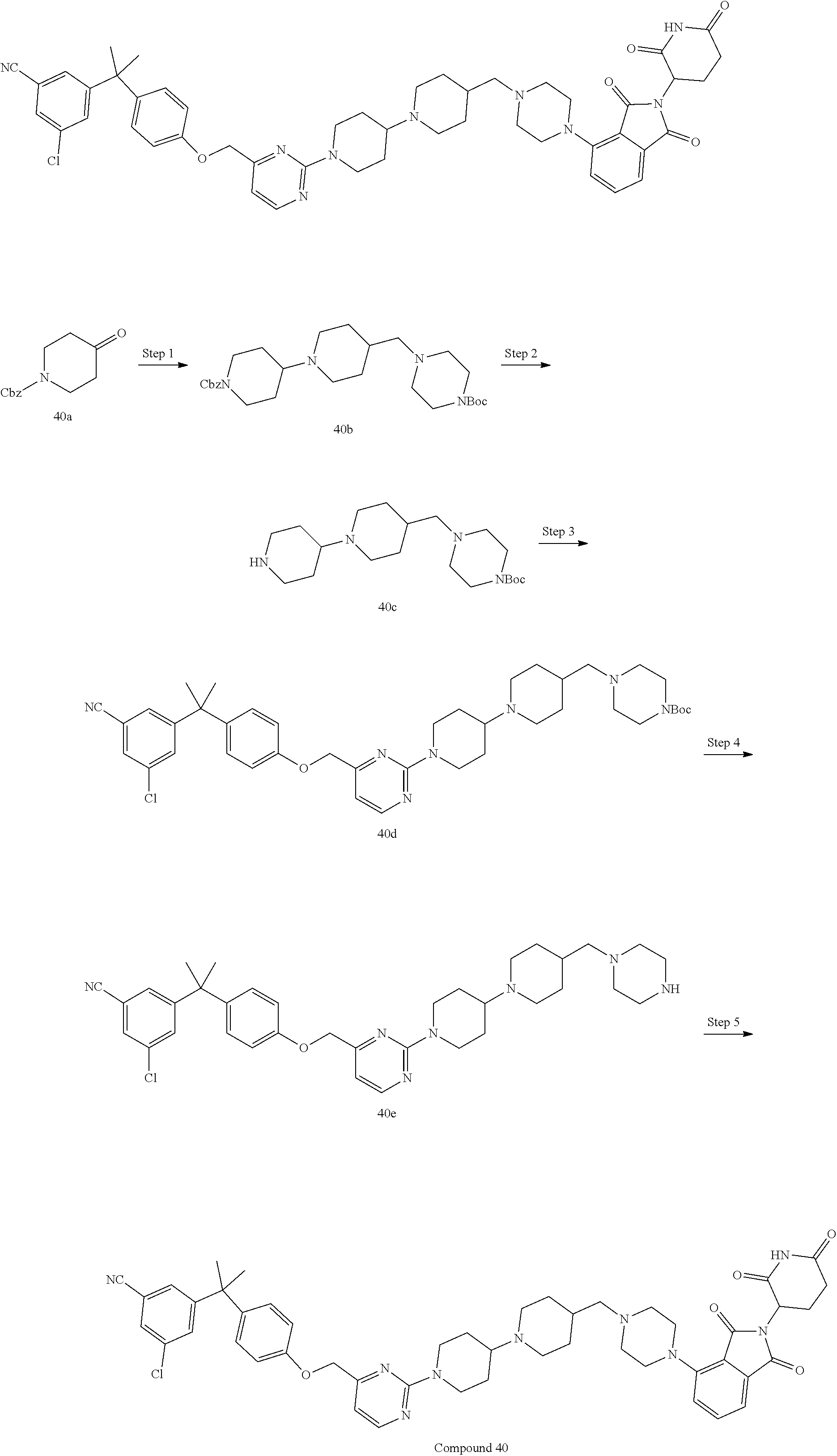

Step 1: benzyl 4-((4-tert-butoxycarbonyl)piperazin-1-yl)methyl)-[1,4′-bipiperidine]-1′-carboxylate(40b)

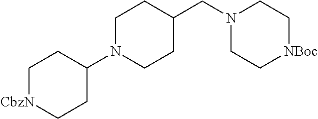 [SMILES: CC(C)(C)OC(=O)N1CCN(CC2CCN(C3CCN(C(=O)OCc4ccccc4)CC3)CC2)CC1]

Tert-butyl 4-(piperidin-4-ylmethyl)piperazine-1-carboxylate (see WO 2020201080 for the synthetic method) (300 mg, 1.06 mmol) and benzyl 4-oxopiperidine-1-carboxylate (40a) (350 mg, 1.5 mmol) were added to 20 mL of dichloromethane, respectively, and the mixture was reacted at ambient temperature for 2 h. Sodium triacetoxyborohydride (317 mg, 1.5 mmol) was added, and the resulting mixture was reacted at room temperature for 12 h. To the reaction liquid were added 50 mL of dichloromethane and 50 mL of 1 mol/L aqueous sodium hydroxide solution. The organic layer was separated, dried over anhydrous sodium sulfate, and concentrated under reduced pressure. The crude product was separated and purified by silica gel column chromatography (petroleum ether/ethyl acetate (v/v)=10:1-1:1), to afford 40b (400 mg, yield: 75%).

LCMS m/z=501.4 [M+H]⁺

### Step 2: tert-butyl 4-([1,4′-bipiperidin]-4-ylmethyl)piperazine-1-carboxylate (40c)

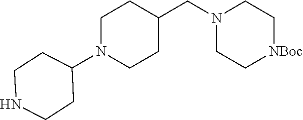 [SMILES: CC(C)(C)OC(=O)N1CCN(CC2CCN(C3CCNCC3)CC2)CC1]

40b (400 mg, 0.8 mmol) was dissolved in methanol (10 mL), and 10% palladium on carbon (100 mg) was added. The mixture was reacted at room temperature under hydrogen atmosphere for 16 h. The reaction liquid was filtered, and the filtrate was concentrated under reduced pressure, to afford crude 40c (200 mg).

Step 3: tert-butyl 4-((1′-(4-((4-(2-(3-chloro-5-cyanophenyl)propan-2-yl)phenoxy)methyl)pyrimidin-2-yl)-[1,4′-bipiperidin]-4-yl)methyl)piperazine-1-carboxylate (40d)

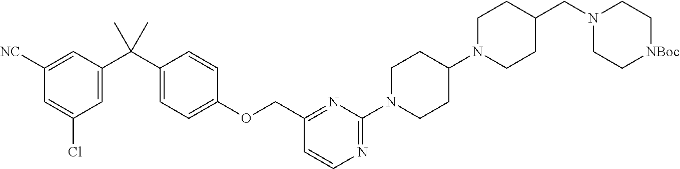 [SMILES: CC(C)(C)OC(=O)N1CCN(CC2CCN(C3CCN(c4nccc(COc5ccc(C(C)(C)c6cc(Cl)cc(C#N)c6)cc5)n4)CC3)CC2)CC1]

9d (200 mg, 0.5 mmol) was dissolved in 5 mL of DMF, and the above crude tert-butyl 4-([1,4′-bipiperidin]-4-ylmethyl)piperazine-1-carboxylate (40c) (200 mg) and solid potassium bicarbonate (138 mg, 1.38 mmol) were successively added. The mixture was warmed to 75° C. and reacted for 4 h. The mixture was cooled to room temperature, and 30 mL of water was added. The resulting mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 40d (250 mg, yield from compound 9d: 69%).

LCMS m/z=728.4 [M+1]⁺

### Step 4: 3-chloro-5-(2-(4-((2-(4-(piperazin-1-ylmethyl)-[1,4′-bipiperidin]-1′-yl)pyrimidin-4-yl)methoxy)phenyl)propan-2-yl)benzonitrile (40e)

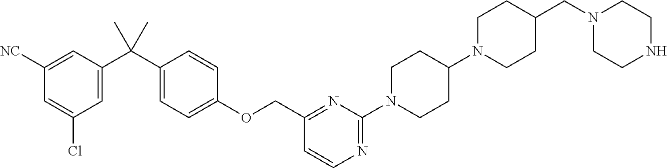 [SMILES: CC(C)(c1ccc(OCc2ccnc(N3CCC(N4CCC(CN5CCNCC5)CC4)CC3)n2)cc1)c1cc(Cl)cc(C#N)c1]

40d (250 mg, 0.34 mmol) was dissolved in 8 mL of dichloromethane, and 2 mL of trifluoroacetic acid was added. The mixture was stirred at room temperature for 2 h. The reaction system was concentrated under reduced pressure, and 30 mL of dichloromethane was added. The mixture was adjusted to pH 10 with a saturated sodium bicarbonate solution, extracted with 30 mL of dichloromethane, dried over anhydrous sodium sulfate, and concentrated under reduced pressure, to afford crude 40e (200 mg).

### Step 5: 3-chloro-5-(2-(4-((2-(4-((4-(2-(2,6-dioxopiperidin-3-yl)-1,3-dioxoisoindolin-4-yl)piperazin-1-yl)methyl)-[1,4′-bipiperidin]-1′-yl)pyrimidin-4-yl)methoxy)phenyl)propan-2-yl)benzonitrile (compound 40) trifluoroacetate

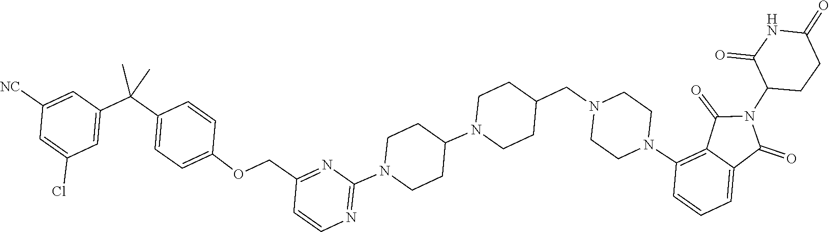 [SMILES: CC(C)(c1ccc(OCc2ccnc(N3CCC(N4CCC(CN5CCN(c6cccc7c6C(=O)N(C6CCC(=O)NC6=O)C7=O)CC5)CC4)CC3)n2)cc1)c1cc(Cl)cc(C#N)c1]

The above crude 40e (100 mg) was dissolved in 5 mL of DMSO, and 0.1 mL of DIPEA and 2-(2,6-dioxopiperidin-3-yl)-4-fluoroisoindoline-1,3-dione (see WO 2017197056 for the synthetic method) (0.1 g, 0.36 mmol) were added. The mixture was warmed to 85° C. and reacted for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 40 trifluoroacetate (30 mg).

¹H NMR (400 MHz, DMSO-d₆) δ 11.08 (s, 1H), 8.40 (d, 1H), 7.87-7.73 (m, 2H), 7.67-7.62 (m, 1H), 7.57-7.39 (m, 3H), 7.23-7.10 (m, 2H), 7.00-6.90 (m, 2H), 6.75 (d, 1H), 5.11 (dd, 1H), 4.99 (s, 2H), 4.90-4.75 (m, 2H), 4.10-3.65 (m, 4H), 3.64-3.36 (m, 2H), 3.36-2.79 (m, 12H), 2.71-2.51 (m, 2H), 2.28-1.88 (m, 6H), 1.76-1.34 (m, 10H).

LCMS m/z=884.4 [M+1]⁺

### Example 41

### 3-chloro-5-(2-(4-((2-(4-((4-(2-(2,6-dioxopiperidin-3-yl)-1,3-dioxoisoindolin-5-yl)piperazin-1-yl)methyl)piperidin-1-yl)pyrimidin-4-yl)methoxy)phenyl)propan-2-yl)benzonitrile (Compound 41) trifluoroacetate

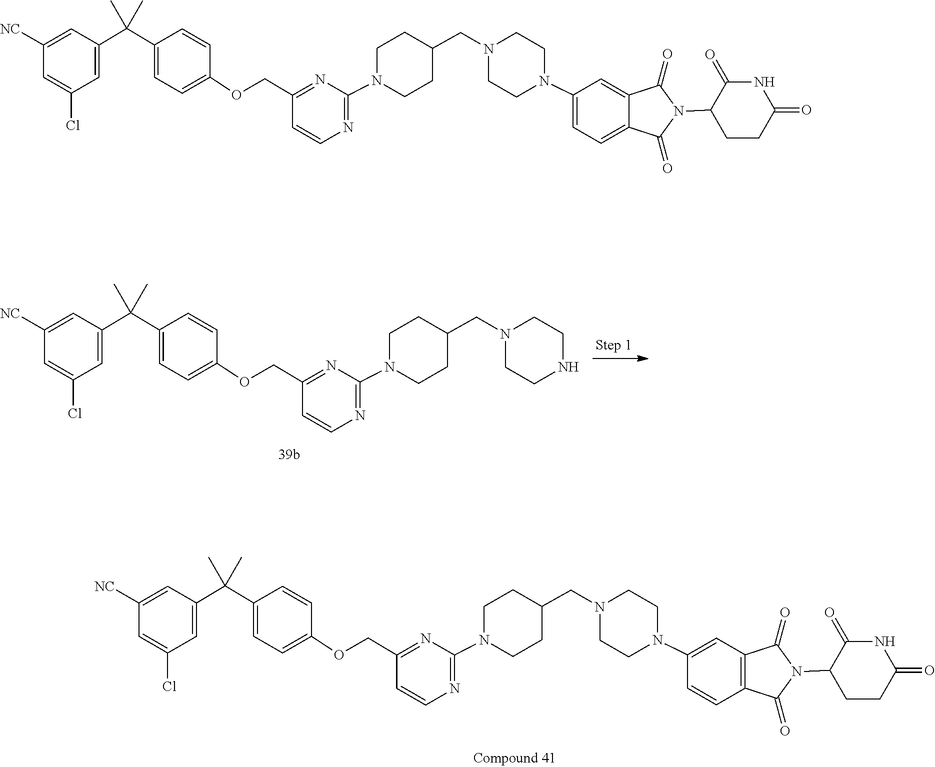 [SMILES: CC(C)(c1ccc(OCc2ccnc(N3CCC(CN4CCN(c5ccc6c(c5)C(=O)N(C5CCC(=O)NC5=O)C6=O)CC4)CC3)n2)cc1)c1cc(Cl)cc(C#N)c1 ; CC(C)(c1ccc(OCc2ccnc(N3CCC(CN4CCNCC4)CC3)n2)cc1)c1cc(Cl)cc(C#N)c1 ; CC(C)(c1ccc(OCc2ccnc(N3CCC(CN4CCN(c5ccc6c(c5)C(=O)N(C5CCC(=O)NC5=O)C6=O)CC4)CC3)n2)cc1)c1cc(Cl)cc(C#N)c1]

The above crude 39b (90 mg) was dissolved in 5 mL of DMSO, and 0.1 mL of DIPEA and 2-(2,6-dioxopiperidin-3-yl)-5-fluoroisoindoline-1,3-dione (see WO 2017197056 for the synthetic method) (0.1 g, 0.36 mmol) were added. The mixture was warmed to 85° C. and reacted for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 41 trifluoroacetate (21 mg).

¹H NMR (400 MHz, DMSO-d₆) δ 11.09 (s, 1H), 8.37 (d, 1H), 7.87-7.74 (m, 2H), 7.67-7.63 (m, 1H), 7.57-7.47 (m, 2H), 7.42-7.34 (m, 1H), 7.24-7.12 (m, 2H), 7.00-6.90 (m, 2H), 6.70 (d, 1H), 5.11 (dd, 1H), 4.98 (s, 2H), 4.75-4.59 (m, 2H), 4.39-4.04 (m, 2H), 3.75-3.05 (m, 8H), 3.05-2.80 (m, 3H), 2.68-2.52 (m, 2H), 2.27-2.11 (m, 1H), 2.10-1.98 (m, 1H), 1.93-1.77 (m, 2H), 1.65 (s, 6H), 1.27-1.09 (m, 2H).

LCMS m/z=801.3 [M+1]⁺

### Example 42

### 3-chloro-5-(2-(4-((2-(4-((4-(2-(2,6-dioxopiperidin-3-yl)-1,3-dioxoisoindolin-5-yl)piperazin-1-yl)methyl)-[1,4′-bipiperidin]-1′-yl)pyrimidin-4-yl)methoxy)phenyl)propan-2-yl)benzonitrile (Compound 42) trifluoroacetate

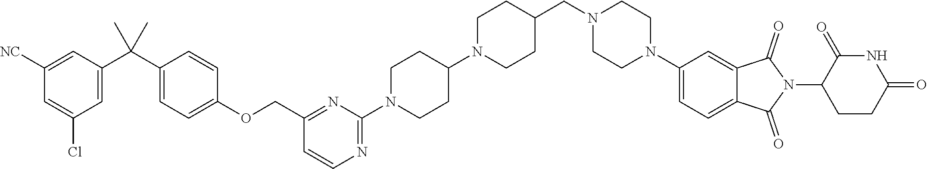 [SMILES: CC(C)(c1ccc(OCc2ccnc(N3CCC(N4CCC(CN5CCN(c6ccc7c(c6)C(=O)N(C6CCC(=O)NC6=O)C7=O)CC5)CC4)CC3)n2)cc1)c1cc(Cl)cc(C#N)c1]

The above crude 40e (100 mg) was dissolved in 5 mL of DMSO, and 0.1 mL of DIPEA and 2-(2,6-dioxopiperidin-3-yl)-5-fluoroisoindoline-1,3-dione (see WO 2017197056 for the synthetic method) (0.1 g, 0.36 mmol) were added. The mixture was warmed to 85° C. and reacted for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 42 trifluoroacetate (25 mg).

¹H NMR (400 MHz, DMSO-d₆) δ 11.08 (s, 1H), 8.40 (d, 1H), 7.89-7.73 (m, 2H), 7.68-7.62 (m, 1H), 7.58-7.47 (m, 2H), 7.42-7.34 (m, 1H), 7.24-7.12 (m, 2H), 7.00-6.91 (m, 2H), 6.75 (d, 1H), 5.10 (dd, 1H), 4.99 (s, 2H), 4.92-4.75 (m, 2H), 3.80-3.46 (m, 7H), 3.46-3.08 (m, 6H), 3.06-2.82 (m, 5H), 2.66-2.52 (m, 2H), 2.26-1.96 (m, 6H), 1.74-1.39 (m, 10H).

LCMS m/z=884.4 [M+1]⁺

### Example 43

### (2S,4R)-1-((S)-2-(2-(4-((4-(4-((4-(2-(3-chloro-5-cyanophenyl)propan-2-yl)phenoxy)methyl)pyrimidin-2-yl)piperazin-1-yl)methyl)-[1,4′-bipiperidin]-1′-yl)acetamido)-3,3-dimethylbutanoyl)-4-hydroxy-N-((S)-1-(4-(4-methylthiazol-5-yl)phenyl)ethyl)pyrrolidine-2-carboxamide (Compound 43) trifluoroacetate

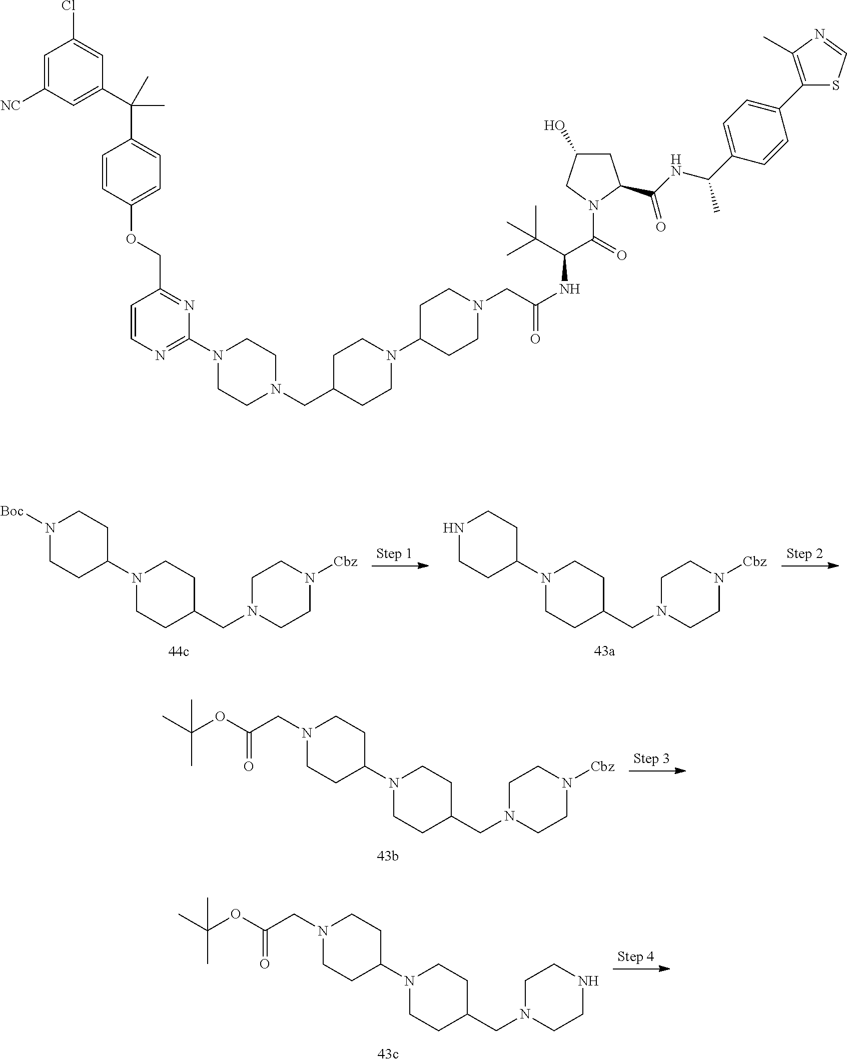 [SMILES: Cc1ncsc1-c1ccc([C@H](C)NC(=O)[C@@H]2C[C@@H](O)CN2C(=O)[C@@H](NC(=O)CN2CCC(N3CCC(CN4CCN(c5nccc(COc6ccc(C(C)(C)c7cc(Cl)cc(C#N)c7)cc6)n5)CC4)CC3)CC2)C(C)(C)C)cc1 ; CC(C)(C)OC(=O)N1CCC(N2CCC(CN3CCN(C(=O)OCc4ccccc4)CC3)CC2)CC1 ; O=C(OCc1ccccc1)N1CCN(CC2CCN(C3CCNCC3)CC2)CC1 ; CC(C)(C)OC(=O)CN1CCC(N2CCC(CN3CCN(C(=O)OCc4ccccc4)CC3)CC2)CC1 ; CC(C)(C)OC(=O)CN1CCC(N2CCC(CN3CCNCC3)CC2)CC1]

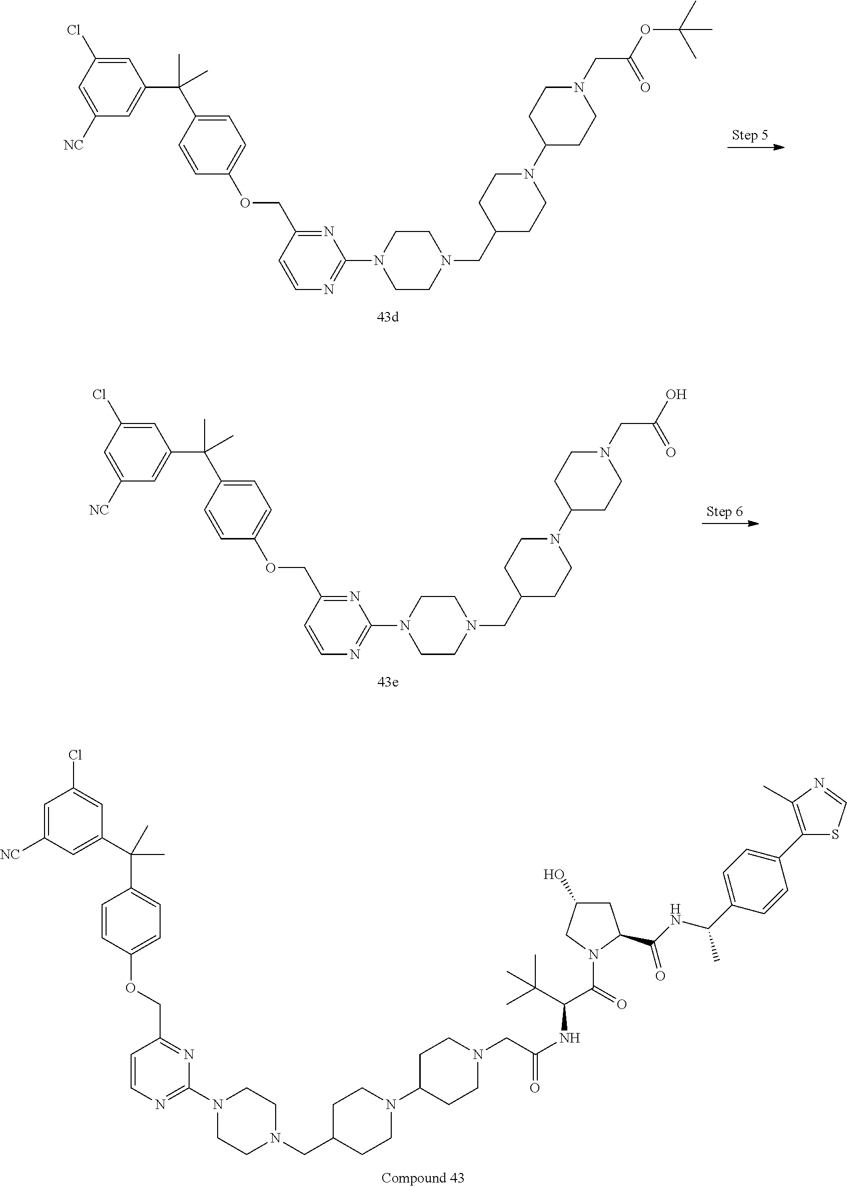 [SMILES: CC(C)(C)OC(=O)CN1CCC(N2CCC(CN3CCN(c4nccc(COc5ccc(C(C)(C)c6cc(Cl)cc(C#N)c6)cc5)n4)CC3)CC2)CC1 ; CC(C)(c1ccc(OCc2ccnc(N3CCN(CC4CCN(C5CCN(CC(=O)O)CC5)CC4)CC3)n2)cc1)c1cc(Cl)cc(C#N)c1 ; Cc1ncsc1-c1ccc([C@H](C)NC(=O)[C@@H]2C[C@@H](O)CN2C(=O)[C@@H](NC(=O)CN2CCC(N3CCC(CN4CCN(c5nccc(COc6ccc(C(C)(C)c7cc(Cl)cc(C#N)c7)cc6)n5)CC4)CC3)CC2)C(C)(C)C)cc1]

### Step 2: benzyl 4-([1,4′-bipiperidin]-4-yl)methyl) piperazine-1-carboxylate (43a)

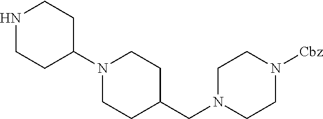 [SMILES: O=C(OCc1ccccc1)N1CCN(CC2CCN(C3CCNCC3)CC2)CC1]

11c (2.0 g, 4.0 mmol) was dissolved in 20 mL of DCM, and 20 mL of trifluoroacetic acid was added. The mixture was stirred at room temperature for 2 h. The reaction liquid was concentrated under reduced pressure, and 50 mL of saturated aqueous sodium bicarbonate solution was added. The mixture was extracted with ethyl acetate (50 mL×3), dried over anhydrous sodium sulfate, and concentrated under reduced pressure, to afford crude 43a (1.0 g).

LCMS m/z=401.3[M+1]⁺

### Step 2: benzyl 4-((1′-(2-(tert-butoxy)-2-oxoethyl)-[1,4′-bipiperidin]-4-yl)methyl)piperazine-1-carboxylate (43b)

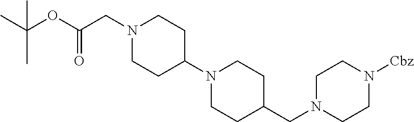 [SMILES: CC(C)(C)OC(=O)CN1CCC(N2CCC(CN3CCN(C(=O)OCc4ccccc4)CC3)CC2)CC1]

The above crude 43a (1.0 g) was dissolved in 10 mL of DMF, and potassium carbonate (0.69 g, 5.0 mmol) and tert-butyl bromoacetate (0.585 g, 3.0 mmol) were added. The mixture was reacted at room temperature for 12 h. To the reaction liquid was added 50 mL of water, and the mixture was extracted with ethyl acetate (50 mL×3), dried over anhydrous sodium sulfate, and concentrated under reduced pressure. The crude product was separated and purified by silica gel column chromatography (ethyl acetate/petroleum ether (v/v)=1:100-1:1), to afford 43b (0.6 g, two-step yield from compound 11c: 29%).

LCMS m/z=515.2 [M+1]⁺

### Step 3: tert-butyl 2-(4-(piperazin-1-ylmethyl)-[1,4′-bipiperidin]-1′-yl)acetate (43c)

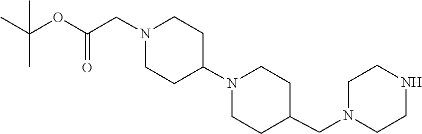 [SMILES: CC(C)(C)OC(=O)CN1CCC(N2CCC(CN3CCNCC3)CC2)CC1]

43b (0.6 g, 1.2 mmol) was dissolved in 20 mL of methanol, and 0.2 g of 10% palladium on carbon was added. The mixture was reacted at room temperature under hydrogen atmosphere for 16 h. The reaction liquid was filtered, and the filtrate was concentrated under reduced pressure, to afford crude 43c (0.44 g).

LCMS m/z=381.3 [M+1]⁺

### Step 4: tert-butyl 2-(4-((4-(4-((4-(2-(3-chloro-5-cyanophenyl)propan-2-yl)phenoxy)methyl)pyrimidin-2-yl)piperazin-1-yl)methyl)-[1,4′-bipiperidin]-1′-yl)acetate (43d)

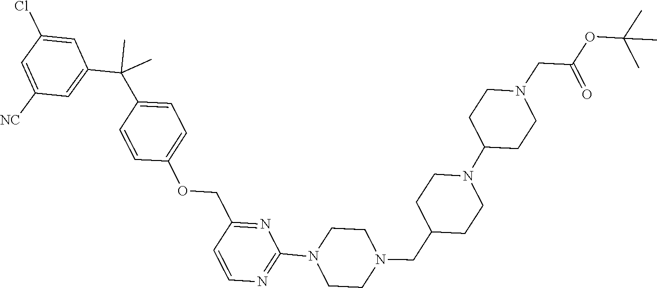 [SMILES: CC(C)(C)OC(=O)CN1CCC(N2CCC(CN3CCN(c4nccc(COc5ccc(C(C)(C)c6cc(Cl)cc(C#N)c6)cc5)n4)CC3)CC2)CC1]

3-chloro-5-(2-(4-((2-chloropyrimidin-4-yl)methoxy)phenyl)propan-2-yl)benzonitrile (9d) (0.26 g, 0.65 mmol) was dissolved in 10 mL of DMF, and solid potassium bicarbonate (105 mg, 1.05 mmol) and the above crude tert-butyl 2-(4-(piperazin-1-ylmethyl)-[1,4′-bipiperidin]-1′-yl)acetate (43c) (0.25 g) were successively added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 43d (200 mg, yield from compound 9d: 42%).

LCMS m/z=742.2 [M+1]⁺

### Step 5: 2-(4-((4-(4-((4-(2-(3-chloro-5-cyanophenyl)propan-2-yl)phenoxy)methyl)pyrimidin-2-yl)piperazin-1-yl)methyl)-[1,4′-bipiperidin]-1′-yl)acetic acid (43e) trifluoroacetate

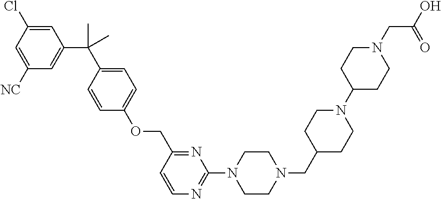 [SMILES: CC(C)(c1ccc(OCc2ccnc(N3CCN(CC4CCN(C5CCN(CC(=O)O)CC5)CC4)CC3)n2)cc1)c1cc(Cl)cc(C#N)c1]

43d (200 mg, 0.27 mmol) was dissolved in 2 mL of DCM, and 2 mL of trifluoroacetic acid was added. The mixture was stirred at room temperature for 12 h. The reaction liquid was concentrated under reduced pressure, to afford crude 43e trifluoroacetate (200 mg).

LCMS m/z=686.3 [M+1]⁺

### Step 6: (2S,4R)-1-((S)-2-(2-(4-((4-(4-((4-(2-(3-chloro-5-cyanophenyl)propan-2-yl)phenoxy)methyl)pyrimidin-2-yl)piperazin-1-yl)methyl)-[1,4′-bipiperidin]-1′-yl)acetamido)-3,3-dimethylbutanoyl)-4-hydroxy-N-((S)-1-(4-(4-methylthiazol-5-yl)phenyl)ethyl)pyrrolidine-2-carboxamide (Compound 43) trifluoroacetate

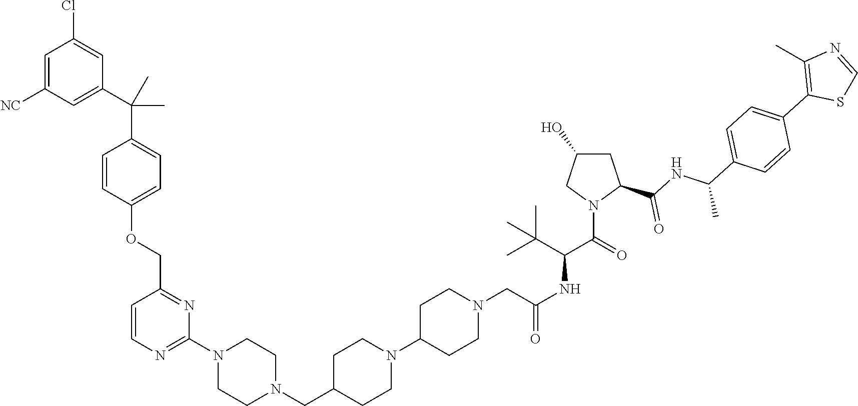 [SMILES: Cc1ncsc1-c1ccc([C@H](C)NC(=O)[C@@H]2C[C@@H](O)CN2C(=O)[C@@H](NC(=O)CN2CCC(N3CCC(CN4CCN(c5nccc(COc6ccc(C(C)(C)c7cc(Cl)cc(C#N)c7)cc6)n5)CC4)CC3)CC2)C(C)(C)C)cc1]

The above crude 43e trifluoroacetate (200 mg) was dissolved in 10 mL of DMF, and DIPEA (250 mg, 1.93 mmol) and HATU (185 mg, 0.49 mmol) were added. The mixture was stirred at room temperature for 30 min, and then (2S,4R)-1-((S)-2-amino-3,3-dimethylbutanoyl)-4-hydroxyl-N-((S)-1-(4-(4-methylthiazol-5-yl)phenyl)ethyl)pyrrolidine-2-carboxamide (see J. Med. Chem. 2019, 62, 1420-1442 for the synthetic method) (170 mg, 0.38 mmol) was added. The resulting mixture was stirred at room temperature for 16 h. To the reaction liquid was added 30 mL of water, and the mixture was extracted with 100 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 43 trifluoroacetate (30 mg).

¹H NMR (400 MHz, CD₃OD) δ 8.97-8.91 (m, 1H), 8.40 (d, 1H), 7.61-7.56 (m, 1H), 7.52-7.35 (m, 6H), 7.20-7.13 (m, 2H), 6.98-6.86 (m, 3H), 5.07-4.97 (m, 3H), 4.68-4.52 (m, 2H), 4.49-4.34 (m, 1H), 4.10-3.83 (m, 4H), 3.82-3.47 (m, 8H), 3.47-3.31 (m, 4H), 3.28-3.06 (m, 7H), 2.48 (s, 3H), 2.47-2.10 (m, 8H), 2.07-1.91 (m, 1H), 1.84-1.69 (m, 2H), 1.67 (s, 6H), 1.64-1.48 (m, 3H), 1.11-1.01 (m, 9H).

LCMS m/z=371.8 [M/3+1]⁺

### Example 44

### 3-chloro-5-(2-(4-((2-(4-(2-(2-(2,6-dioxopiperidin-3-yl)-1,3-dioxoisoindolin-5-yl)-2,9-diazaspiro[5.5]undecan-9-yl)-[1,4′-bipiperidin]-1′-yl)pyrimidin-4-yl)methoxy)phenyl) propan-2-yl)benzonitrile (Compound 44) trifluoroacetate

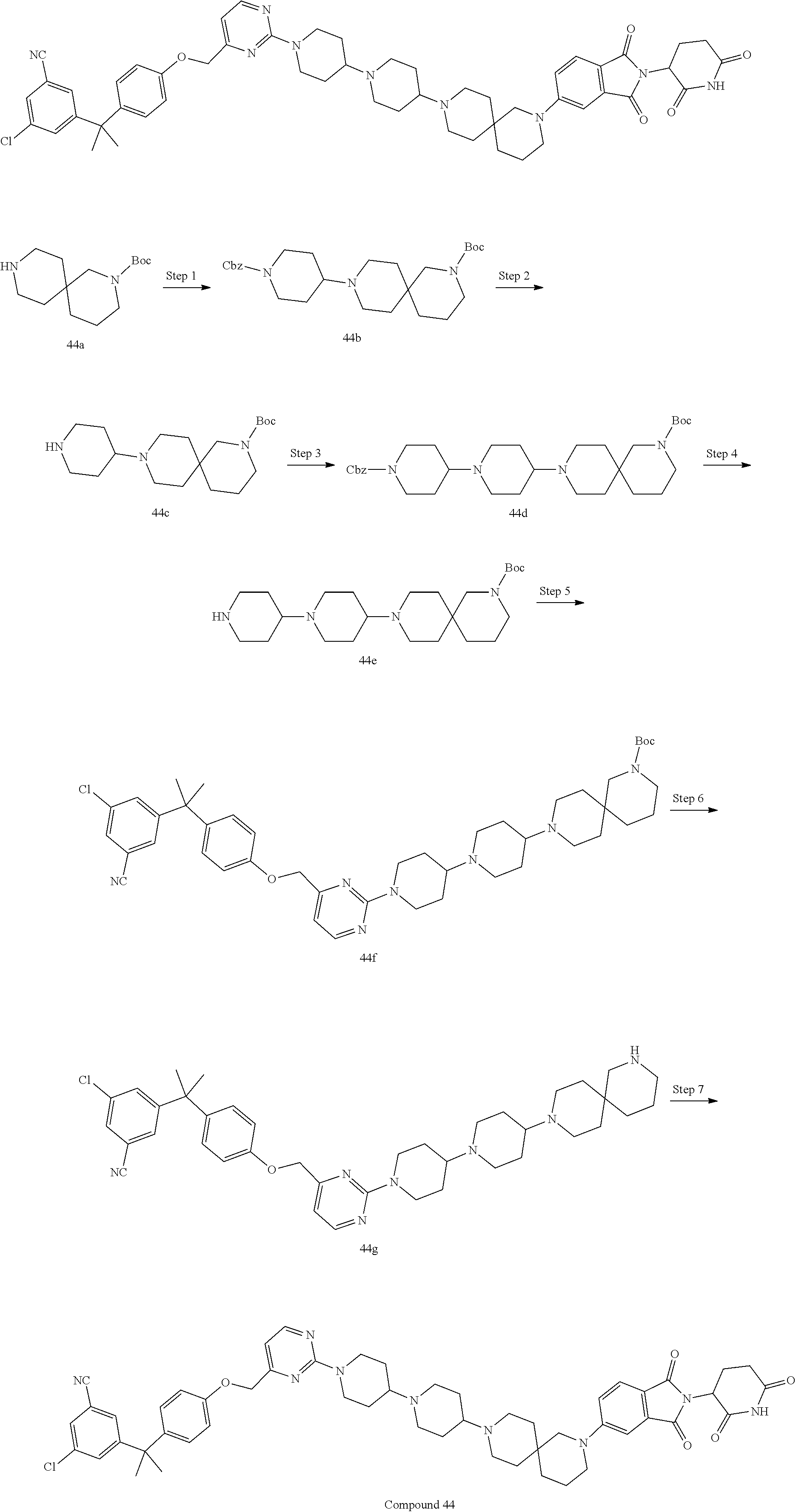

### Step 1: tert-butyl 9-(1-((benzyloxy)carbonyl)piperidin-4-yl)-2,9-diazaspiro[5.5]undecane-2-carboxylate (44b)

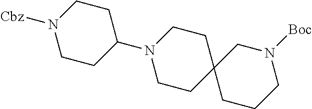 [SMILES: CC(C)(C)OC(=O)N1CCCC2(CCN(C3CCN(C(=O)OCc4ccccc4)CC3)CC2)C1]

44a (2.54 g, 10.00 mmol) was dissolved in 35 mL of DCE, and benzyl 4-oxopiperidine-1-carboxylate (2.80 g, 12.00 mmol) and 2.0 ml of acetic acid were successively added. The mixture was stirred at room temperature for 60 min, and then sodium triacetoxyborohydride (3.18 g, 15.0 mmol) was added. The resulting mixture was reacted at room temperature for 16 h. To the reaction system was slowly added 60 mL of saturated sodium bicarbonate solution, and the mixture was extracted with 200 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 44b (4.0 g, yield: 85%).

LCMS m/z=472.4 [M+1]⁺

### Step 2: tert-butyl 9-(piperidin-4-yl)-2,9-diazaspiro[5.5]undecane-2-carboxylate (44c)

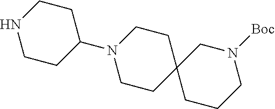 [SMILES: CC(C)(C)OC(=O)N1CCCC2(CCN(C3CCNCC3)CC2)C1]

44b (2.0 g, 4.24 mmol) was dissolved in 10 mL of methanol, and 0.4 g of 10% Pd/C was added. The mixture was stirred at room temperature under hydrogen atmosphere for 3 h. The reaction system was filtered by suction, and the filtrate was concentrated under reduced pressure, to afford crude 44c (1.4 g).

LCMS m/z=338.3 [M+1]⁺

### Step 3: tert-butyl 9-(1′-((benzyloxy)carbonyl)-[1,4′-bipiperidin]-4-yl)-2,9-diazaspiro[5.5]undecane-2-carboxylate (44d)

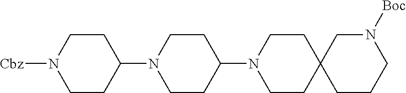 [SMILES: CC(C)(C)OC(=O)N1CCCC2(CCN(C3CCN(C4CCN(C(=O)OCc5ccccc5)CC4)CC3)CC2)C1]

The above crude 44c (0.8 g) was dissolved in 15 mL of DCE, and benzyl 4-oxopiperidine-1-carboxylate (666 mg, 2.86 mmol) and 1.0 mL of acetic acid were successively added. The mixture was stirred at room temperature for 60 min, and then sodium triacetoxyborohydride (1.0 g, 4.72 mmol) was added. The resulting mixture was reacted at room temperature for 16 h. To the reaction system was slowly added 60 mL of saturated sodium bicarbonate solution, and the mixture was extracted with 200 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=20:1), to afford 44d (1.0 g, two-step yield from compound 44b: 74%).

LCMS m/z=555.4 [M+1]⁺

### Step 4: tert-butyl 9-([1,4′-bipiperidin]-4-yl)-2,9-diazaspiro[5.5]undecane-2-carboxylate (44e)

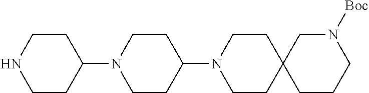 [SMILES: CC(C)(C)OC(=O)N1CCCC2(CCN(C3CCN(C4CCNCC4)CC3)CC2)C1]

44d (0.6 g, 1.08 mmol) was dissolved in 10 mL of methanol, and 0.2 g of 10% Pd/C was added. The mixture was stirred at room temperature under hydrogen atmosphere for 3 h. The reaction system was filtered by suction, and the filtrate was concentrated under reduced pressure, to afford crude 44e (0.46 g).

LCMS m/z=421.3 [M+1]⁺

### Step 5: tert-butyl 9-(1′-(4-((4-(2-(3-chloro-5-cyanophenyl)propan-2-yl)phenoxy)methyl)pyrimidin-2-yl)-[1,4′-bipiperidin]-4-yl)-2,9-diazaspiro[5.5]undecane-2-carboxylate (44f)

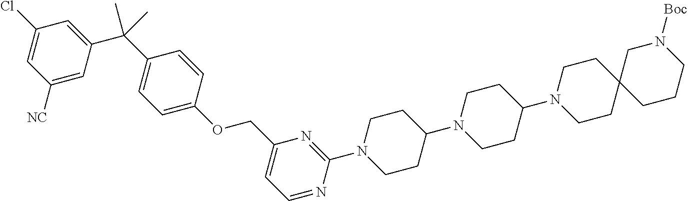 [SMILES: CC(C)(C)OC(=O)N1CCCC2(CCN(C3CCN(C4CCN(c5nccc(COc6ccc(C(C)(C)c7cc(Cl)cc(C#N)c7)cc6)n5)CC4)CC3)CC2)C1]

3-chloro-5-(2-(4-((2-chloropyrimidin-4-yl)methoxy)phenyl)propan-2-yl)benzonitrile (9d) (100 mg, 0.25 mmol) was dissolved in 20 mL of DMF, and the above crude 44e (126 mg) and solid potassium bicarbonate (50 mg, 0.50 mmol) were successively added. The mixture was warmed to 60° C. and reacted for 3 h. The reaction system was cooled to room temperature, and 50 mL of water was added. The mixture was extracted with ethyl acetate (40 mL×3), and the organic phase was washed with water (50 mL×2), dried over anhydrous sodium sulfate, and concentrated under reduced pressure. The crude product was separated and purified by silica gel column chromatography (dichloromethane/methanol (v/v)=20:1), to afford 44f (80 mg, yield from compound 9d: 41%).

LCMS m/z=782.4 [M+1]⁺

### Step 6: 3-(2-(4-((2-(4-(2,9-diazaspiro[5.5]undecan-9-yl)-[1,4′-bipiperidin]-1′-yl)pyrimidin-4-yl)methoxy)phenyl)propan-2-yl)-5-chlorobenzonitrile (44g) trifluoroacetate

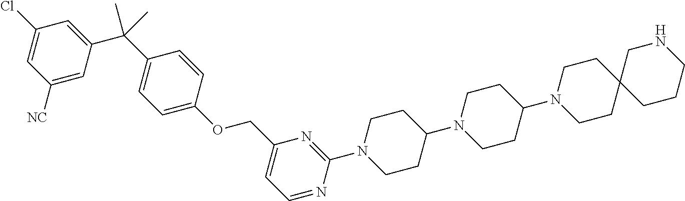 [SMILES: CC(C)(c1ccc(OCc2ccnc(N3CCC(N4CCC(N5CCC6(CCCNC6)CC5)CC4)CC3)n2)cc1)c1cc(Cl)cc(C#N)c1]

44f (80 mg, 0.10 mmol) was dissolved in 10 mL of DCM, and 4 mL of trifluoroacetic acid was added. The mixture was stirred at room temperature for 2 h. The reaction liquid was concentrated under reduced pressure, to afford crude 44g trifluoroacetate (0.10 g).

LCMS m/z=682.5 [M+1]⁺

### Step 7: 3-chloro-5-(2-(4-((2-(4-(2-(2-(2,6-dioxopiperidin-3-yl)-1,3-dioxoisoindolin-5-yl)-2,9-diazaspiro[5.5]undecan-9-yl)-[1,4′-bipiperidin]-1′-yl)pyrimidin-4-yl)methoxy)phenyl) propan-2-yl)benzonitrile (Compound 44) trifluoroacetate

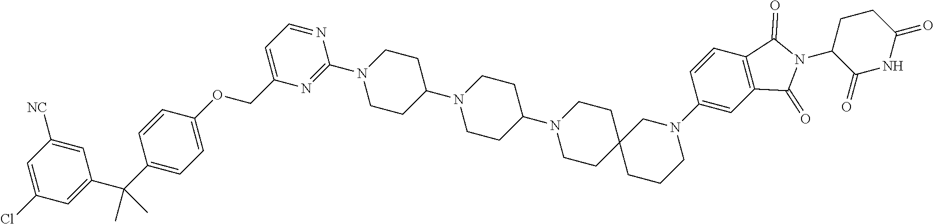 [SMILES: CC(C)(c1ccc(OCc2ccnc(N3CCC(N4CCC(N5CCC6(CCCN(c7ccc8c(c7)C(=O)N(C7CCC(=O)NC7=O)C8=O)C6)CC5)CC4)CC3)n2)cc1)c1cc(Cl)cc(C#N)c1]

The above crude 44g trifluoroacetate (0.1 g) was dissolved in 4 mL of DMSO, and solid sodium bicarbonate (42 mg, 0.50 mmol) was added. The mixture was stirred at room temperature for 10 min, and 0.2 mL of DIPEA and 2-(2,6-dioxopiperidin-3-yl)-5-fluoroisoindoline-1,3-dione (see WO 2017197056 for the synthetic method) (55 mg, 0.20 mmol) were added. The reaction was stirred at 80° C. for 5 h. The reaction liquid was cooled to room temperature, and 50 mL of water was added. The mixture was filtered, and the solid was collected, washed with 10 mL of water, dissolved with 50 mL of DCM, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 44 trifluoroacetate (25 mg).

¹H NMR (400 MHz, DMSO-d₆) δ 11.05 (s, 1H), 8.39 (d, 1H), 7.87-7.82 (m, 1H), 7.72-7.62 (m, 2H), 7.55-7.50 (m, 1H), 7.38-7.32 (m, 1H), 7.30-7.21 (m, 1H), 7.20-7.09 (m, 2H), 7.00-6.90 (m, 2H), 6.75 (d, 1H), 5.06 (dd, 1H), 4.98 (s, 2H), 4.90-4.75 (m, 2H), 3.75-3.25 (m, 10H), 3.15-2.75 (m, 7H), 2.70-2.51 (m, 2H), 2.50-2.46 (m, 2H), 2.45-2.21 (m, 2H), 2.16-1.72 (m, 9H), 1.70-1.22 (m, 10H).

LCMS m/z=938.4 [M+1]⁺.

### Example 45

### 3-chloro-5-(2-(4-((2-(2-((1-(2-(2,6-dioxopiperidin-3-yl)-1,3-dioxoisoindolin-5-yl)piperidin-4-yl)methyl)-2,7-diazaspiro[3.5]nonan-7-yl)pyrimidin-4-yl)methoxy)phenyl)propan-2-yl)benzonitrile (Compound 45)

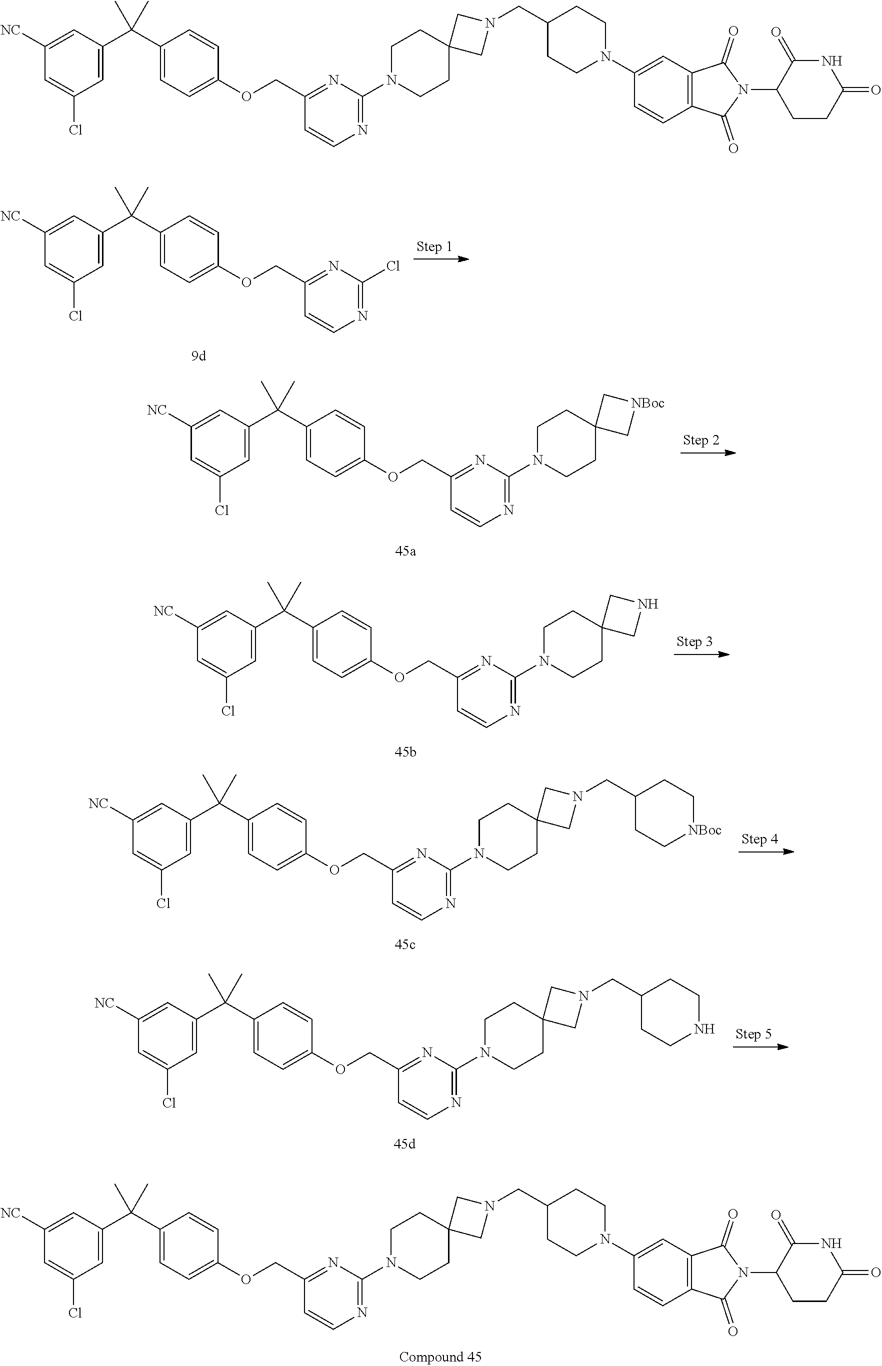

### Step 1: tert-butyl 7-(4-((4-(2-(3-chloro-5-cyanophenyl)propan-2-yl)phenoxy)methyl)pyrimidin-2-yl)-2,7-diazaspiro[3.5]nonane-2-carboxylate (45a)

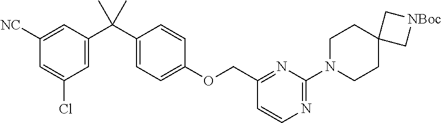 [SMILES: CC(C)(C)OC(=O)N1CC2(CCN(c3nccc(COc4ccc(C(C)(C)c5cc(Cl)cc(C#N)c5)cc4)n3)CC2)C1]

3-chloro-5-(2-(4-((2-chloropyrimidin-4-yl)methoxy)phenyl)propan-2-yl)benzonitrile (9d) (0.25 g, 0.63 mmol) was dissolved in 5 mL of N,N-dimethylformamide, and tert-butyl 2,7-diazaspiro[3.5]nonane-2-carboxylate (0.22 g, 0.97 mmol) and solid potassium bicarbonate (0.16 g, 1.60 mmol) were added, respectively. The mixture was heated to 60° C. and stirred for 2 h. The reaction liquid was cooled to room temperature, and 30 mL of water was added. The mixture was extracted with ethyl acetate (30 mL×3), and the organic phase was washed with 100 mL of saturated sodium chloride solution, dried over anhydrous sodium sulfate, and concentrated under reduced pressure. The crude product was separated and purified by silica gel column chromatography (dichloromethane/methanol (v/v)=99:1), to afford 45a (0.249 g, yield: 67%).

### Step 2: 3-(2-(4-((2-(2,7-diazaspiro[3.5]nonan-7-yl)pyrimidin-4-yl)methoxy)phenyl)propan-2-yl)-5-chlorobenzonitrile (45b) trifluoroacetate

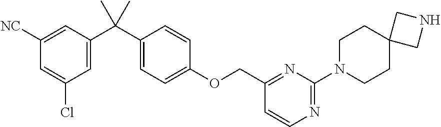 [SMILES: CC(C)(c1ccc(OCc2ccnc(N3CCC4(CC3)CNC4)n2)cc1)c1cc(Cl)cc(C#N)c1]

45a (0.249 g, 0.42 mmol) was dissolved in 2 mL of dichloromethane, and trifluoroacetic acid (3.06 g, 26.84 mmol) was added. The mixture was stirred at room temperature for 2 h. The reaction system was concentrated under reduced pressure, to afford crude 45b trifluoroacetate (0.26 g).

### Step 3: tert-butyl 4-((7-(4-((4-(2-(3-chloro-5-cyanophenyl)propan-2-yl)phenoxy)methyl)pyrimidin-2-yl)-2,7-diazaspiro[3.5]nonan-2-yl)methyl)piperidine-1-carboxylate (45c)

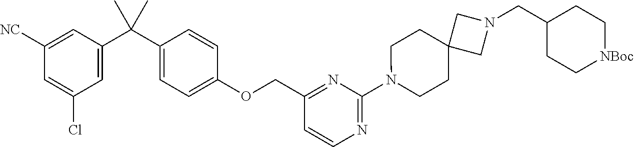 [SMILES: CC(C)(C)OC(=O)N1CCC(CN2CC3(CCN(c4nccc(COc5ccc(C(C)(C)c6cc(Cl)cc(C#N)c6)cc5)n4)CC3)C2)CC1]

The above crude 45b trifluoroacetate (0.13 g) was dissolved in 5 mL of dichloromethane, and solid sodium bicarbonate (0.11 g, 1.30 mmol) was added. The mixture was stirred at room temperature for 0.5 h, and then to the reaction system were respectively added tert-butyl 4-formylpiperidine-1-carboxylate (0.064 g, 0.30 mmol) and sodium triacetoxyborohydride (0.11 g, 0.52 mmol). The resulting mixture was reacted at room temperature for 16 h. To the reaction system was added 5 mL of saturated sodium bicarbonate solution to quench the reaction. The mixture was extracted with ethyl acetate (10 mL×3), and the organic phase was washed with 20 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 45c (0.16 g, two-step yield from compound 45a: >99%).

LCMS m/z=685.3 [M+1]⁺

### Step 4: 3-chloro-5-(2-(4-((2-(2-(piperidin-4-ylmethyl)-2,7-diazaspiro[3.5]nonan-7-yl)pyrimidin-4-yl)methoxy)phenyl)propan-2-yl)benzonitrile (45d) trifluoroacetate

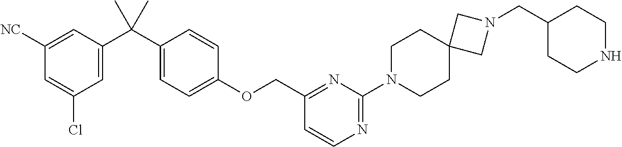 [SMILES: CC(C)(c1ccc(OCc2ccnc(N3CCC4(CC3)CN(CC3CCNCC3)C4)n2)cc1)c1cc(Cl)cc(C#N)c1]

45c (0.16 g, 0.23 mmol) was dissolved in 2 mL of dichloromethane, and trifluoroacetic acid (3.06 g, 26.84 mmol) was added. The mixture was stirred at room temperature for 2 h. The reaction system was concentrated under reduced pressure, to afford crude 45d trifluoroacetate (0.20 g).

Step 5: 3-chloro-5-(2-(4-((2-(2-((1-(2-(2,6-dioxopiperidin-3-yl)-1,3-dioxoisoindolin-5-yl)piperidin-4-yl)methyl)-2,7-diazaspiro[3.5]nonan-7-yl)pyrimidin-4-yl)methoxy)phenyl) propan-2-yl)benzonitrile (Compound 45)

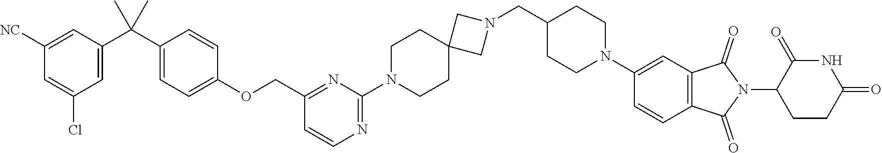 [SMILES: CC(C)(c1ccc(OCc2ccnc(N3CCC4(CC3)CN(CC3CCN(c5ccc6c(c5)C(=O)N(C5CCC(=O)NC5=O)C6=O)CC3)C4)n2)cc1)c1cc(Cl)cc(C#N)c1]

To a reaction flask were successively added the above crude 45d trifluoroacetate (0.20 g), 2-(2,6-dioxopiperidin-3-yl)-5-fluoroisoindoline-1,3-dione (see WO 2017197056 for the synthetic method) (0.14 g, 0.51 mmol) and diisopropylethylamine (0.21 mL). 5 mL of dimethyl sulfoxide was added, and then the mixture was warmed to 80° C. and reacted for 4 h. The reaction liquid was cooled to room temperature, and 20 mL of water was added. The mixture was extracted with dichloromethane (20 mL×3), and the organic phase was washed with 20 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 45 trifluoroacetate (67 mg). To compound 45 trifluoroacetate (40 mg) were successively added dichloromethane (5 mL), methanol (1 mL) and 5 mL of ammonia water, and the mixture was stirred at room temperature for 5 min. The organic phase was then separated, and the aqueous phase was extracted with dichloromethane (10 mL×2). The organic phases were combined, dried over anhydrous sodium sulfate, and concentrated under reduced pressure, to afford compound 45 (25 mg, two-step yield from compound 45c: 22%).

¹H NMR (400 MHz, CDCl₃) δ 8.29 (d, 1H), 7.66 (d, 1H), 7.46-7.40 (m, 2H), 7.39-7.34 (m, 1H), 7.28 (d, 1H), 7.12-7.00 (m, 3H), 6.92-6.83 (m, 2H), 6.68 (d, 1H), 4.99-4.88 (m, 3H), 4.02-3.87 (m, 2H), 3.82-3.68 (m, 4H), 3.15 (br.s, 4H), 3.03-2.64 (m, 5H), 2.57-2.37 (m, 2H), 2.18-2.08 (m, 1H), 1.94-1.74 (m, 6H), 1.74-1.50 (m, 7H), 1.38-1.23 (m, 2H).

LCMS m/z=841.3 [M+1]⁺.

### Example 46

### 3-chloro-5-(2-(4-((2-(4-(1-(2-(2-(2,6-dioxopiperidin-3-yl)-1,3-dioxoisoindolin-5-yl)-2-azaspiro[3.5]nonan-7-yl)piperidin-4-yl)piperazin-1-yl)pyrimidin-4-yl)methoxy)phenyl) propan-2-yl)benzonitrile (Compound 46) trifluoroacetate

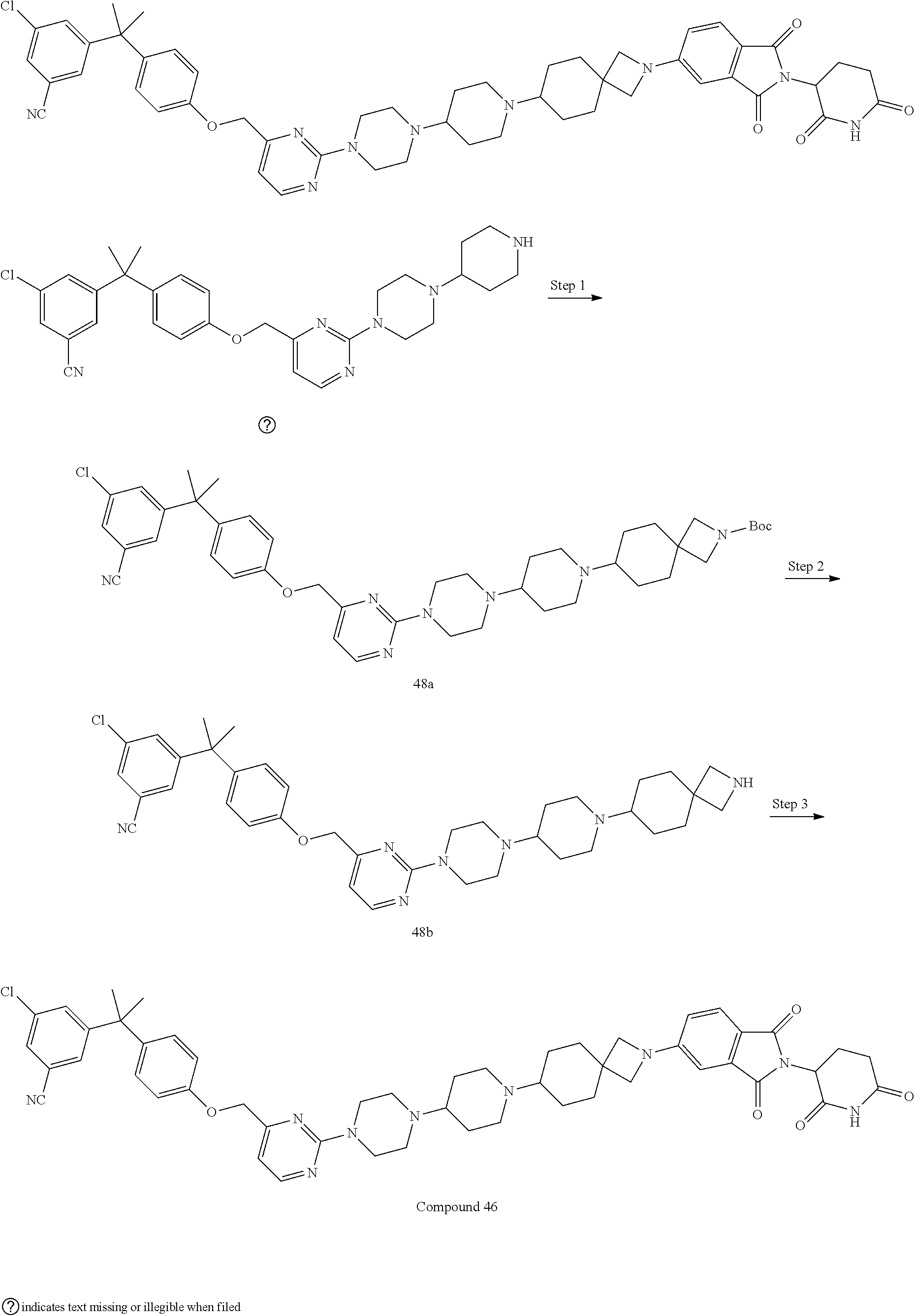 [SMILES: CC(C)(c1ccc(OCc2ccnc(N3CCN(C4CCN(C5CCC6(CC5)CN(c5ccc7c(c5)C(=O)N(C5CCC(=O)NC5=O)C7=O)C6)CC4)CC3)n2)cc1)c1cc(Cl)cc(C#N)c1 ; CC(C)(c1ccc(OCc2ccnc(N3CCN(C4CCNCC4)CC3)n2)cc1)c1cc(Cl)cc(C#N)c1 ; CC(C)(C)OC(=O)N1CC2(CCC(N3CCC(N4CCN(c5nccc(COc6ccc(C(C)(C)c7cc(Cl)cc(C#N)c7)cc6)n5)CC4)CC3)CC2)C1 ; CC(C)(c1ccc(OCc2ccnc(N3CCN(C4CCN(C5CCC6(CC5)CNC6)CC4)CC3)n2)cc1)c1cc(Cl)cc(C#N)c1 ; CC(C)(c1ccc(OCc2ccnc(N3CCN(C4CCN(C5CCC6(CC5)CN(c5ccc7c(c5)C(=O)N(C5CCC(=O)NC5=O)C7=O)C6)CC4)CC3)n2)cc1)c1cc(Cl)cc(C#N)c1]

### Step 1: tert-butyl 7-(4-(4-(4-((4-(2-(3-chloro-5-cyanophenyl)propan-2-yl)phenoxy)methyl)pyrimidin-2-yl)piperazin-1-yl)piperidin-1-yl)-2-azaspiro[3.5]nonane-2-carboxylate (46a)

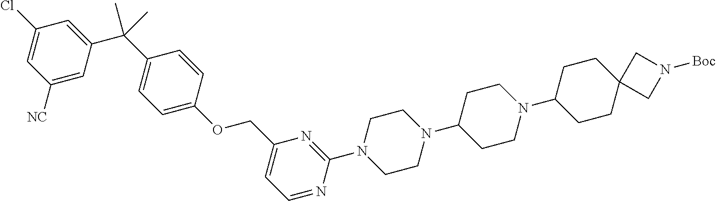 [SMILES: CC(C)(C)OC(=O)N1CC2(CCC(N3CCC(N4CCN(c5nccc(COc6ccc(C(C)(C)c7cc(Cl)cc(C#N)c7)cc6)n5)CC4)CC3)CC2)C1]

The above crude 34c trifluoroacetate (120 mg) was dissolved in 15 mL of DCE, and solid sodium bicarbonate (54 mg, 0.64 mmol) was added. The mixture was stirred at room temperature for 20 min, and then tert-butyl 7-oxo-2-azaspiro[3.5]nonane-2-carboxylate (77 mg, 0.32 mmol) and 0.05 mL of acetic acid were successively added. The mixture was stirred at room temperature for 60 min, and then sodium triacetoxyborohydride (68 mg, 0.32 mmol) was added. The resulting mixture was reacted at room temperature for 16 h. To the reaction system was slowly added 20 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=8:1), to afford 46a (60 mg, two-step yield from compound 34b: 52%).

LCMS m/z=754.4 [M+1]⁺

### Step 2: 3-(2-(4-((2-(4-(1-(2-azaspiro[3.5]nonan-7-yl)piperidin-4-yl)piperazin-1-yl)pyrimidin-4-yl)methoxy)phenyl)propan-2-yl)-5-chlorobenzonitrile (46b) trifluoroacetate

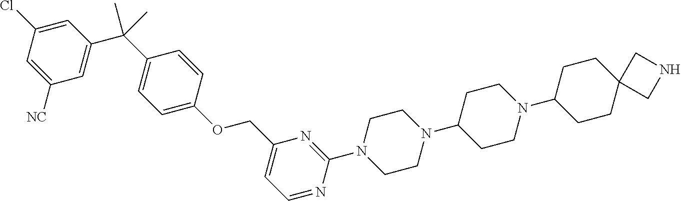 [SMILES: CC(C)(c1ccc(OCc2ccnc(N3CCN(C4CCN(C5CCC6(CC5)CNC6)CC4)CC3)n2)cc1)c1cc(Cl)cc(C#N)c1]

46a (60 mg, 0.08 mmol) was dissolved in 5 mL of DCM, and 2 mL of trifluoroacetic acid was added. The mixture was stirred at room temperature for 2 h. The reaction liquid was concentrated under reduced pressure, to afford crude 46b trifluoroacetate (75 mg).

LCMS m/z=654.4 [M+1]⁺

### Step 3: 3-chloro-5-(2-(4-((2-(4-(1-(2-(2-(2,6-dioxopiperidin-3-yl)-1,3-dioxoisoindolin-5-yl)-2-azaspiro[3.5]nonan-7-yl)piperidin-4-yl)piperazin-1-yl)pyrimidin-4-yl)methoxy)phenyl)propan-2-yl)benzonitrile (Compound 46) trifluoroacetate

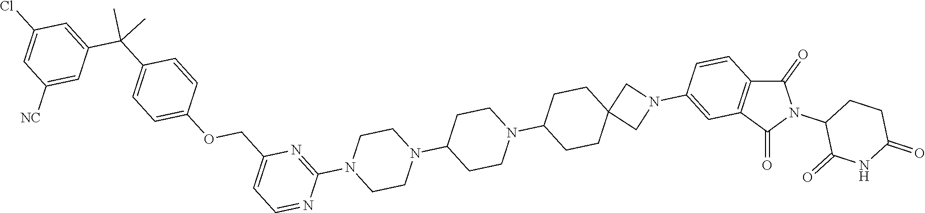 [SMILES: CC(C)(c1ccc(OCc2ccnc(N3CCN(C4CCN(C5CCC6(CC5)CN(c5ccc7c(c5)C(=O)N(C5CCC(=O)NC5=O)C7=O)C6)CC4)CC3)n2)cc1)c1cc(Cl)cc(C#N)c1]

The above crude 46b trifluoroacetate (75 mg) was dissolved in 4 mL of DMSO, and solid sodium bicarbonate (35 mg, 0.42 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (45 mg, 0.16 mmol) were added. The reaction was stirred at 80° C. for 5 h. The reaction liquid was cooled to room temperature, and 50 mL of water was added. The mixture was filtered, and the solid was collected, washed with 10 mL of water, dissolved with 50 mL of DCM, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 46 trifluoroacetate (8 mg).

¹H NMR (400 MHz, DMSO-d₆) δ 11.05 (s, 1H), 8.50-8.41 (m, 1H), 7.87-7.82 (m, 1H), 7.69-7.61 (m, 2H), 7.56-7.49 (m, 1H), 7.21-7.11 (m, 2H), 6.99-6.91 (m, 2H), 6.88-6.80 (m, 1H), 6.80-6.72 (m, 1H), 6.68-6.60 (m, 1H), 5.12-4.96 (m, 3H), 3.85-3.55 (m, 6H), 3.28-2.78 (m, 13H), 2.70-2.52 (m, 2H), 2.40-2.20 (m, 2H), 2.15-1.85 (m, 7H), 1.70-1.42 (m, 10H).

LCMS m/z=910.3 [M+1]⁺.

### Example 47

### 3-chloro-5-(2-(4-((2-(3-(4-((4-(2-(2,6-dioxopiperidin-3-yl)-1,3-dioxoisoindolin-5-yl) piperazin-1-yl)methyl)piperidin-1-yl)azetidin-1-yl)pyrimidin-4-yl)methoxy)phenyl) propan-2-yl)benzonitrile (Compound 47) trifluoroacetate

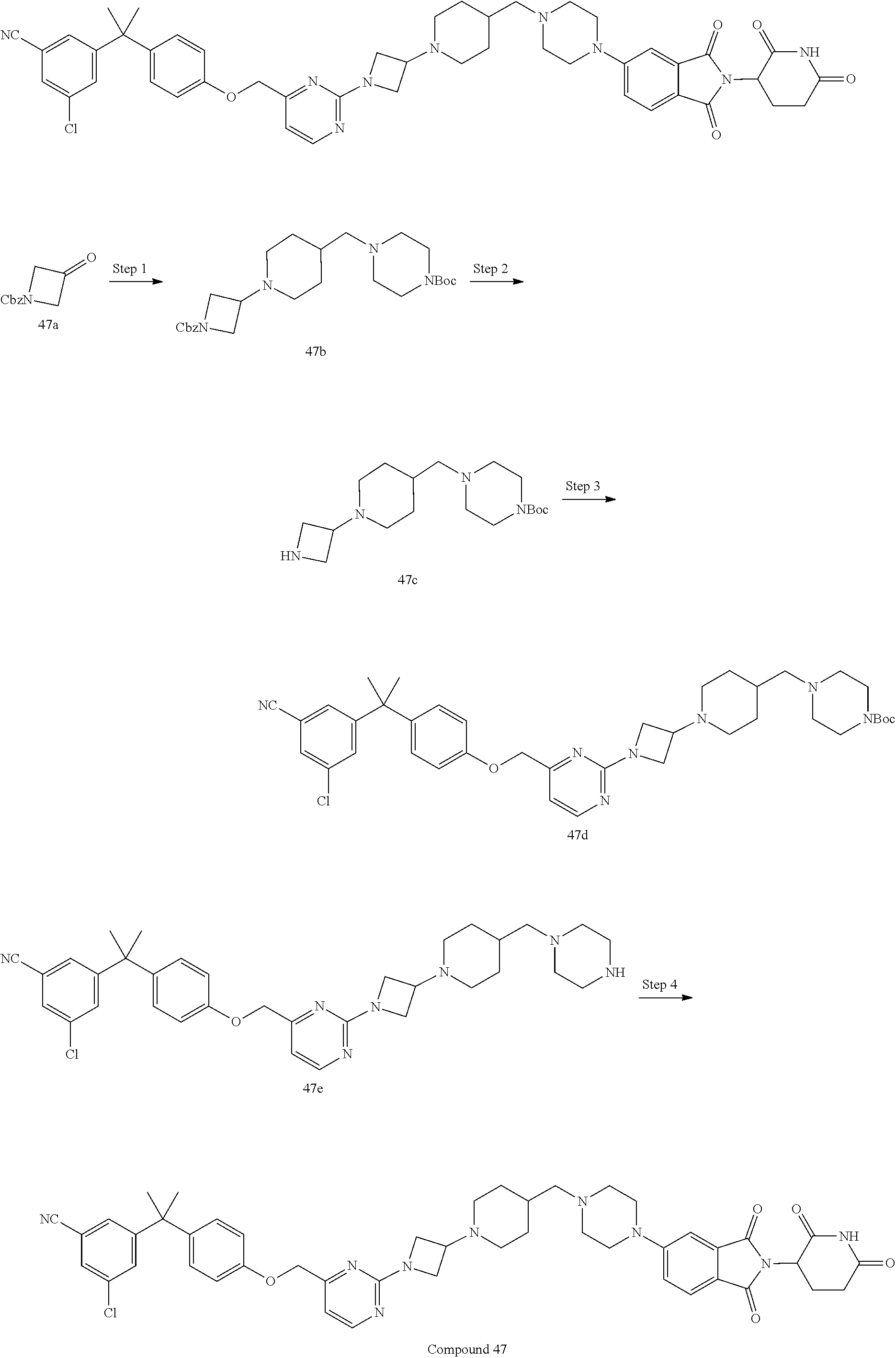

### Step 1: tert-butyl 4-((1-(1-((benzyloxy)carbonyl)azetidin-3-yl)piperidin-4-yl)methyl) piperazine-1-carboxylate (47b)

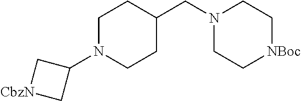 [SMILES: CC(C)(C)OC(=O)N1CCN(CC2CCN(C3CN(C(=O)OCc4ccccc4)C3)CC2)CC1]

Tert-butyl 4-(piperidin-4-ylmethyl)piperazine-1-carboxylate (see WO 2020201080 for the synthetic method) (300 mg, 1.06 mmol) and benzyl 3-oxaazetidine-1-carboxylate (307 mg, 1.5 mmol) were added to 20 mL of dichloromethane, respectively, and the mixture was stirred at room temperature for 2 h. Sodium triacetoxyborohydride (317 mg, 1.5 mmol) was then added, and the resulting mixture was reacted at room temperature for 12 h. To the reaction liquid were added 50 mL of dichloromethane and 50 mL of 1 mol/L aqueous sodium hydroxide solution. The organic phase was separated, dried over anhydrous sodium sulfate, and concentrated under reduced pressure. The crude product was separated and purified by silica gel column chromatography (dichloromethane/methanol (v/v)=10:1), to afford 47b (350 mg, yield: 70%).

LCMS m/z=473.3 [M+1]⁺

### Step 2: tert-butyl 4-((1-(azetidin-3-yl)piperidin-4-yl)methyl)piperazine-1-carboxylate (47c)

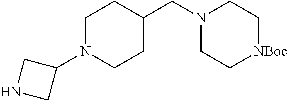 [SMILES: CC(C)(C)OC(=O)N1CCN(CC2CCN(C3CNC3)CC2)CC1]

47b (350 mg, 0.74 mmol) was dissolved in methanol (10 mL), and 100 mg of 10% palladium on carbon was added. The mixture was stirred at room temperature under hydrogen atmosphere for 16 h. The reaction system was filtered by suction, and the filtrate was concentrated under reduced pressure, to afford crude 47c (220 mg).

LCMS m/z=339.3 [M+1]⁺

### Step 3: tert-butyl 4-((1-(1-(4-((4-(2-(3-chloro-5-cyanophenyl)propan-2-yl)phenoxy)methyl)pyrimidin-2-yl)azetidin-3-yl)piperidin-4-yl)methyl)piperazine-1-carboxylate (47d)

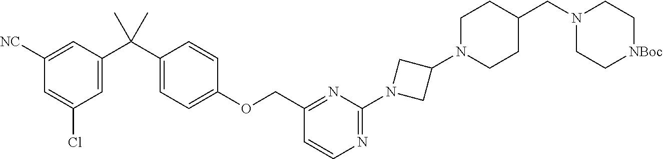 [SMILES: CC(C)(C)OC(=O)N1CCN(CC2CCN(C3CN(c4nccc(COc5ccc(C(C)(C)c6cc(Cl)cc(C#N)c6)cc5)n4)C3)CC2)CC1]

3-chloro-5-(2-(4-((2-chloropyrimidin-4-yl)methoxy)phenyl)propan-2-yl)benzonitrile (9d) (200 mg, 0.5 mmol) was dissolved in 5 mL of DMF, and the above crude 47c (220 mg) and solid potassium bicarbonate (138 mg, 1.38 mmol) were successively added. The mixture was warmed to 75° C. and reacted for 4 h. The mixture was cooled to room temperature, and 30 mL of water was added. The resulting mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 47d (280 mg, yield from compound 9d: 81%).

LCMS m/z=700.3 [M+1]⁺

### Step 4: 3-chloro-5-(2-(4-((2-(3-(4-(piperazin-1-ylmethyl)piperidin-1-yl)azetidin-1-yl)pyrimidin-4-yl)methoxy)phenyl)propan-2-yl)benzonitrile (47e)

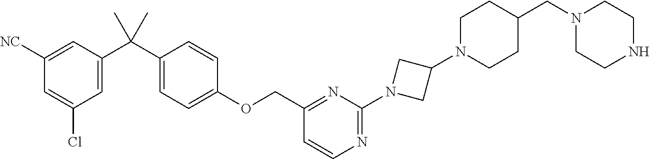 [SMILES: CC(C)(c1ccc(OCc2ccnc(N3CC(N4CCC(CN5CCNCC5)CC4)C3)n2)cc1)c1cc(Cl)cc(C#N)c1]

47d (280 mg, 0.4 mmol) was dissolved in 8 mL of dichloromethane, and 2 mL of trifluoroacetic acid was added. The mixture was stirred at room temperature for 2 h. The reaction system was concentrated under reduced pressure, and 30 mL of dichloromethane was added. The mixture was adjusted to pH 9 with a saturated sodium bicarbonate solution, extracted with 30 mL of dichloromethane, dried over anhydrous sodium sulfate, and concentrated under reduced pressure, to afford crude 47e (220 mg).

### Step 5: 3-chloro-5-(2-(4-((2-(3-(4-((4-(2-(2,6-dioxopiperidin-3-yl)-1,3-dioxoisoindolin-5-yl)piperazin-1-yl)methyl)piperidin-1-yl)azetidin-1-yl)pyrimidin-4-yl)methoxy)phenyl) propan-2-yl)benzonitrile (Compound 47) trifluoroacetate

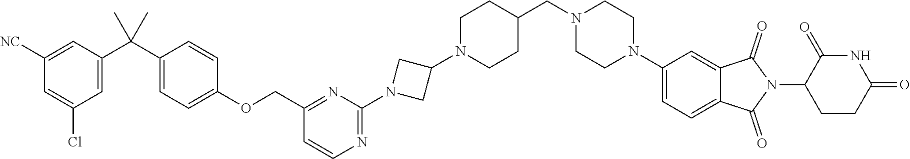 [SMILES: CC(C)(c1ccc(OCc2ccnc(N3CC(N4CCC(CN5CCN(c6ccc7c(c6)C(=O)N(C6CCC(=O)NC6=O)C7=O)CC5)CC4)C3)n2)cc1)c1cc(Cl)cc(C#N)c1]

The above crude 47e (110 mg) was dissolved in 5 mL of DMSO, and 0.1 mL of DIPEA and 2-(2,6-dioxopiperidin-3-yl)-5-fluoroisoindoline-1,3-dione (see WO 2017197056 for the synthetic method) (0.1 g, 0.36 mmol) were added. The mixture was warmed to 85° C. and reacted for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 47 trifluoroacetate (30 mg).

¹H NMR (400 MHz, DMSO-d₆) δ 11.08 (s, 1H), 8.43 (d, 1H), 7.88-7.73 (m, 2H), 7.69-7.61 (m, 1H), 7.57-7.45 (m, 2H), 7.42-7.32 (m, 1H), 7.23-7.12 (m, 2H), 7.00-6.84 (m, 3H), 5.17-4.96 (m, 3H), 4.40-4.08 (m, 5H), 3.77-3.51 (m, 5H), 3.35-2.75 (m, 10H), 2.70-2.51 (m, 2H), 2.30-1.93 (m, 4H), 1.64 (s, 6H), 1.55-1.30 (m, 2H).

LCMS m/z=856.3 [M+1]⁺

### Example 48

### 3-chloro-5-(2-(4-((2-(3-(4-((4-(2-(2,6-dioxopiperidin-3-yl)-1,3-dioxoisoindolin-4-yl)piperazin-1-yl)methyl)piperidin-1-yl)azetidin-1-yl)pyrimidin-4-yl)methoxy)phenyl) propan-2-yl)benzonitrile (Compound 48) trifluoroacetate

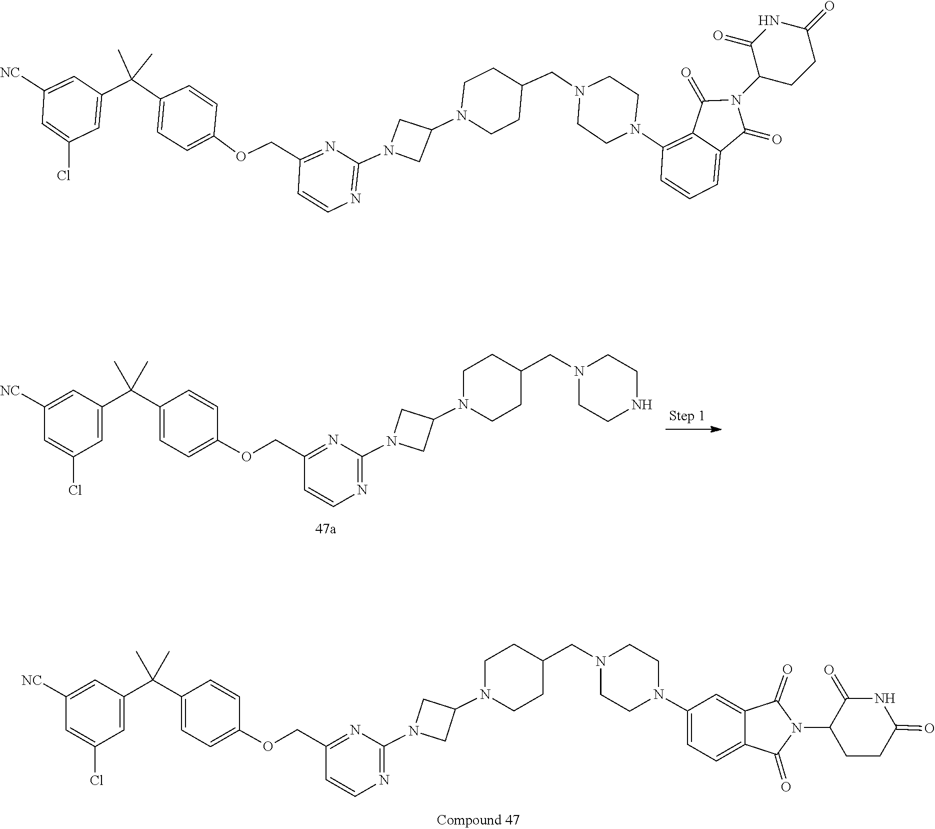 [SMILES: CC(C)(c1ccc(OCc2ccnc(N3CC(N4CCC(CN5CCN(c6cccc7c6C(=O)N(C6CCC(=O)NC6=O)C7=O)CC5)CC4)C3)n2)cc1)c1cc(Cl)cc(C#N)c1 ; CC(C)(c1ccc(OCc2ccnc(N3CC(N4CCC(CN5CCNCC5)CC4)C3)n2)cc1)c1cc(Cl)cc(C#N)c1 ; CC(C)(c1ccc(OCc2ccnc(N3CC(N4CCC(CN5CCN(c6ccc7c(c6)C(=O)N(C6CCC(=O)NC6=O)C7=O)CC5)CC4)C3)n2)cc1)c1cc(Cl)cc(C#N)c1]

The above crude 47e (110 mg) was dissolved in 5 mL of DMSO, and 0.1 mL of DIPEA and 2-(2,6-dioxopiperidin-3-yl)-4-fluoroisoindoline-1,3-dione (see WO 2017197056 for the synthetic method) (0.1 g, 0.36 mmol) were added. The mixture was warmed to 85° C. and reacted for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 48 trifluoroacetate (20 mg).

¹H NMR (400 MHz, DMSO-d₆) δ 11.08 (s, 1H), 8.43 (d, 1H), 7.88-7.73 (m, 2H), 7.67-7.62 (m, 1H), 7.55-7.51 (m, 1H), 7.50-7.40 (m, 2H), 7.22-7.12 (m, 2H), 7.00-6.92 (m, 2H), 6.88 (d, 1H), 5.11 (dd, 1H), 5.00 (s, 2H), 4.40-4.12 (m, 5H), 4.02-3.61 (m, 6H), 3.46-3.06 (m, 6H), 3.00-2.78 (m, 3H), 2.71-2.52 (m, 2H), 2.30-1.96 (m, 4H), 1.65 (s, 6H), 1.55-1.33 (m, 2H).

LCMS m/z=856.3 [M+1]⁺

### Example 49

### 3-chloro-5-(2-(4-((2-(4-(1-((1-(2-(2,6-dioxopiperidin-3-yl)-1,3-dioxoisoindolin-5-yl)piperidin-4-yl)methyl)piperidin-4-yl)piperazin-1-yl)pyrimidin-4-yl)methoxy)phenyl) propan-2-yl)benzonitrile (Compound 49) trifluoroacetate

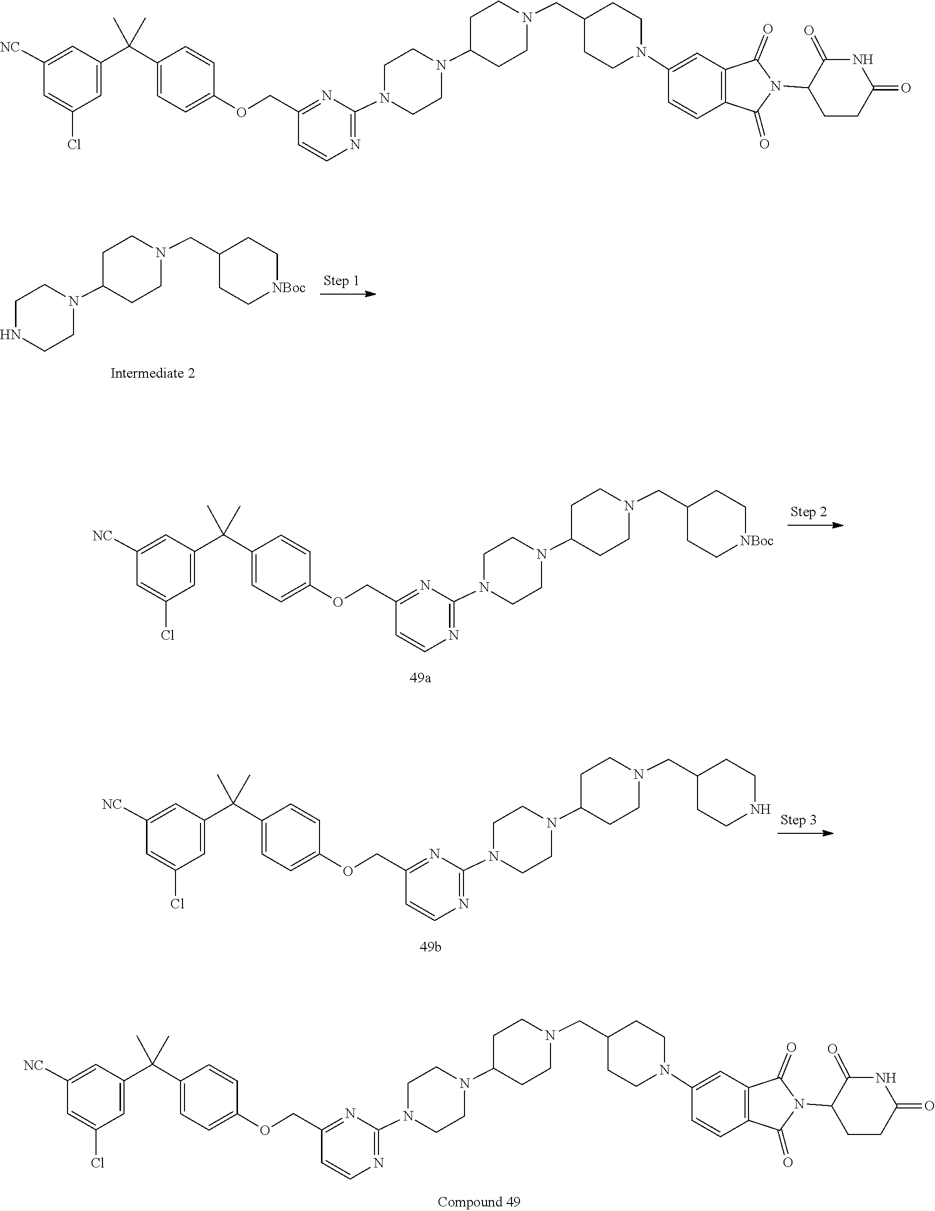 [SMILES: CC(C)(c1ccc(OCc2ccnc(N3CCN(C4CCN(CC5CCN(c6ccc7c(c6)C(=O)N(C6CCC(=O)NC6=O)C7=O)CC5)CC4)CC3)n2)cc1)c1cc(Cl)cc(C#N)c1 ; CC(C)(C)OC(=O)N1CCC(CN2CCC(N3CCNCC3)CC2)CC1 ; CC(C)(C)OC(=O)N1CCC(CN2CCC(N3CCN(c4nccc(COc5ccc(C(C)(C)c6cc(Cl)cc(C#N)c6)cc5)n4)CC3)CC2)CC1 ; CC(C)(c1ccc(OCc2ccnc(N3CCN(C4CCN(CC5CCNCC5)CC4)CC3)n2)cc1)c1cc(Cl)cc(C#N)c1 ; CC(C)(c1ccc(OCc2ccnc(N3CCN(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

### Step 1: tert-butyl 4-((4-(4-(4-((4-(2-(3-chloro-5-cyanophenyl)propan-2-yl)phenoxy)methyl)pyrimidin-2-yl)piperazin-1-yl)piperidin-1-yl)methyl)piperidine-1-carboxylate (49a)

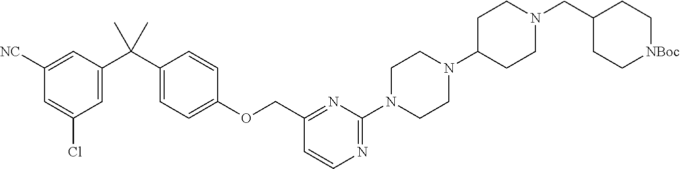 [SMILES: CC(C)(C)OC(=O)N1CCC(CN2CCC(N3CCN(c4nccc(COc5ccc(C(C)(C)c6cc(Cl)cc(C#N)c6)cc5)n4)CC3)CC2)CC1]

3-chloro-5-(2-(4-((2-chloropyrimidin-4-yl)methoxy)phenyl)propan-2-yl)benzonitrile (9d) (450 mg, 1.13 mmol) was dissolved in 10 mL of DMF, and the above crude tert-butyl 4-((4-(piperazin-1-yl)piperidin-1-yl)methyl)piperidine-1-carboxylate (intermediate 2) (460 mg) and solid potassium bicarbonate (230 mg, 2.30 mmol) were successively added. The mixture was warmed to 90° C. and reacted for 4 h. The reaction liquid was cooled to room temperature, and 30 mL of water was added. The mixture was extracted twice with 50 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 49a (500 mg, yield from compound 9d: 61%).

LCMS m/z=728.3 [M+1]⁺

### Step 2: 3-chloro-5-(2-(4-((2-(4-(1-(piperidin-4-ylmethyl)piperidin-4-yl)piperazin-1-yl)pyrimidin-4-yl)methoxy)phenyl)propan-2-yl)benzonitrile (49b) trifluoroacetate

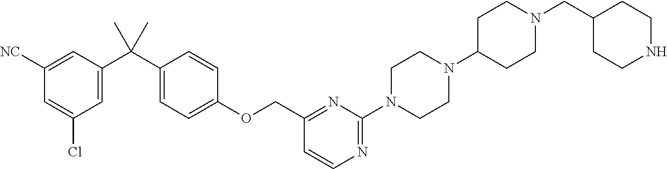 [SMILES: CC(C)(c1ccc(OCc2ccnc(N3CCN(C4CCN(CC5CCNCC5)CC4)CC3)n2)cc1)c1cc(Cl)cc(C#N)c1]

49a (280 mg, 0.385 mmol) was dissolved in 2 mL of DCM, and 2 mL of trifluoroacetic acid was added. The mixture was stirred at room temperature for 4 h. The reaction system was concentrated under reduced pressure, to afford crude 49b trifluoroacetate (510 mg).

LCMS m/z=628.2 [M+1]⁺

### Step 3: 3-chloro-5-(2-(4-((2-(4-(1-((1-(2-(2,6-dioxopiperidin-3-yl)-1,3-dioxoisoindolin-5-yl)piperidin-4-yl)methyl)piperidin-4-yl)piperazin-1-yl)pyrimidin-4-yl)methoxy)phenyl) propan-2-yl)benzonitrile (Compound 49) trifluoroacetate

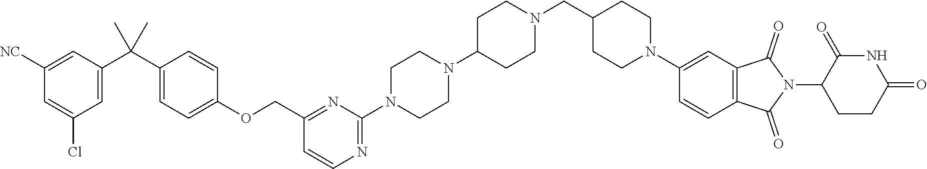 [SMILES: CC(C)(c1ccc(OCc2ccnc(N3CCN(C4CCN(CC5CCN(c6ccc7c(c6)C(=O)N(C6CCC(=O)NC6=O)C7=O)CC5)CC4)CC3)n2)cc1)c1cc(Cl)cc(C#N)c1]

The above crude 49b trifluoroacetate (100 mg) was dissolved in 5 mL of DMSO, and 1 mL of DIPEA and 2-(2,6-dioxopiperidin-3-yl)-5-fluoroisoindoline-1,3-dione (see WO 2017197056 for the synthetic method) (39 mg, 0.14 mmol) were added. The reaction was stirred at 80° C. for 8 h. The reaction liquid was cooled to room temperature, and 20 mL of saturated sodium bicarbonate solution was slowly added. The mixture was extracted twice with 60 mL of ethyl acetate, and the organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 49 trifluoroacetate (20 mg).

¹H NMR (400 MHz, CDCl₃) δ 8.24 (d, 1H), 7.99 (br.s, 1H), 7.60 (d, 1H), 7.42-7.19 (m, 4H), 7.07-6.91 (m, 3H), 6.87-6.75 (m, 2H), 6.64 (d, 1H), 4.96-4.80 (m, 3H), 3.96-3.66 (m, 6H), 3.00-2.47 (m, 11H), 2.38-2.00 (m, 4H), 1.98-1.68 (m, 4H), 1.68-1.40 (m, 9H), 1.30-1.10 (m, 4H).

LCMS m/z=442.8 [M/2+1]⁺

### Example 50

### (2S,4R)-1-((S)-2-(2-(4-((4-(4-(4-((4-(2-(3-chloro-5-cyanophenyl)propan-2-yl)phenoxy)methyl)pyrimidin-2-yl)piperazin-1-yl)piperidin-1-yl)methyl)piperidin-1-yl)acetamido)-3,3-dimethylbutanoyl)-4-hydroxy-N-((S)-1-(4-(4-methylthiazol-5-yl)phenyl)ethyl)pyrrolidine-2-carboxamide (Compound 50) trifluoroacetate

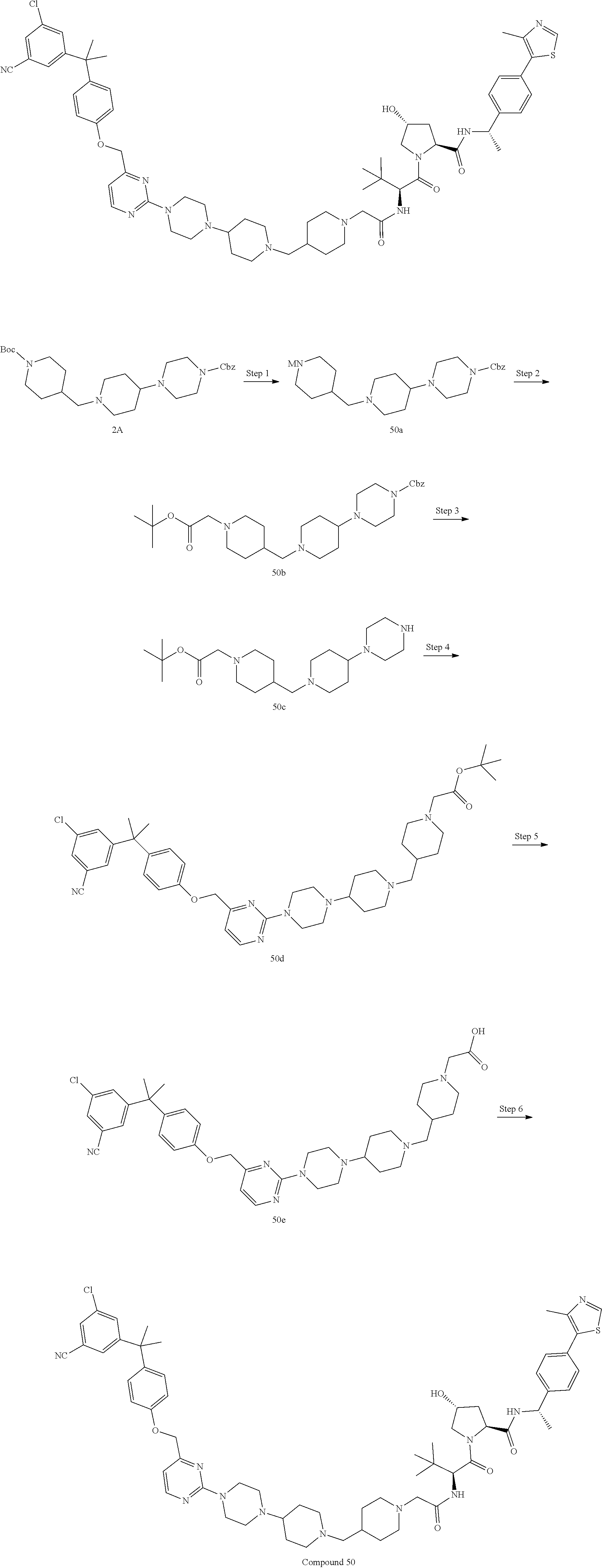

### Step 1: benzyl 4-(1-(piperidin-4-ylmethyl)piperidin-4-yl)piperazine-1-carboxylate (50a)

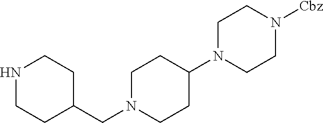 [SMILES: O=C(OCc1ccccc1)N1CCN(C2CCN(CC3CCNCC3)CC2)CC1]

2A (2.0 g, 4.0 mmol) was dissolved in 20 mL of DCM, and 20 mL of trifluoroacetic acid was added. The mixture was stirred at room temperature for 2 h. The reaction liquid was concentrated under reduced pressure, and 50 mL of saturated aqueous sodium bicarbonate solution was added. The mixture was extracted with ethyl acetate (50 mL×3), dried over anhydrous sodium sulfate, and concentrated under reduced pressure, to afford crude 50a (1.2 g).

LCMS m/z=401.3 [M+1]⁺

### Step 2: benzyl 4-(1-((1-(2-(tert-butoxy)-2-oxoethyl)piperidin-4-yl)methyl)piperidin-4-yl)piperazine-1-carboxylate (50b)

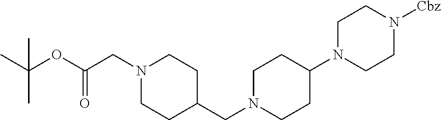 [SMILES: CC(C)(C)OC(=O)CN1CCC(CN2CCC(N3CCN(C(=O)OCc4ccccc4)CC3)CC2)CC1]

The above crude 50a (1.0 g) was dissolved in 10 mL of DMF, and solid potassium carbonate (0.69 g, 5.0 mmol) and tert-butyl bromoacetate (0.585 g, 3.0 mmol) were added. The mixture was reacted at room temperature for 12 h. The reaction liquid was added to 50 mL of water, and the mixture was extracted with ethyl acetate (50 mL×3), dried over anhydrous sodium sulfate, and concentrated under reduced pressure. The crude product was separated and purified by silica gel column chromatography (ethyl acetate/petroleum ether (v/v)=1:100-1:1), to afford 50b (0.6 g, two-step yield from compound 2A: 35%).

LCMS m/z=515.2 [M+1]⁺

### Step 3: tert-butyl 2-(4-((4-(piperazin-1-yl)piperidin-1-yl)methyl)piperidin-1-yl)acetate (50c)

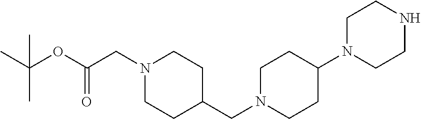 [SMILES: CC(C)(C)OC(=O)CN1CCC(CN2CCC(N3CCNCC3)CC2)CC1]

50b (0.6 g, 1.2 mmol) was dissolved in 20 mL of methanol, and 0.2 g of 10% palladium on carbon was added. The mixture was reacted at room temperature under hydrogen atmosphere for 16 h. The reaction system was filtered by suction, and the filtrate was concentrated under reduced pressure, to afford crude 50c (0.44 g).

LCMS m/z=381.3 [M+1]⁺

### Step 4: tert-butyl 2-(4-((4-(4-(4-((4-(2-(3-chloro-5-cyanophenyl)propan-2-yl)phenoxy)methyl)pyrimidin-2-yl)piperazin-1-yl)piperidin-1-yl)methyl)piperidin-1-yl)acetate (50d)

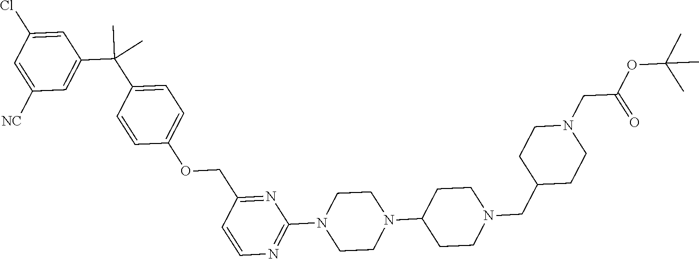 [SMILES: CC(C)(C)OC(=O)CN1CCC(CN2CCC(N3CCN(c4nccc(COc5ccc(C(C)(C)c6cc(Cl)cc(C#N)c6)cc5)n4)CC3)CC2)CC1]

3-chloro-5-(2-(4-((2-chloropyrimidin-4-yl)methoxy)phenyl)propan-2-yl)benzonitrile (9d) (0.26 g, 0.65 mmol) was dissolved in 10 ml of DMF, and solid potassium bicarbonate (105 mg, 1.05 mmol) and the above crude 50c (0.25 g) were successively added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 50d (200 mg, yield from compound 9d: 42%).

LCMS m/z=742.2 [M+1]⁺

### Step 5: 2-(4-((4-(4-(4-((4-(2-(3-chloro-5-cyanophenyl)propan-2-yl)phenoxy)methyl)pyrimidin-2-yl)piperazin-1-yl)piperidin-1-yl)methyl)piperidin-1-yl)acetic acid (50e) trifluoroacetate

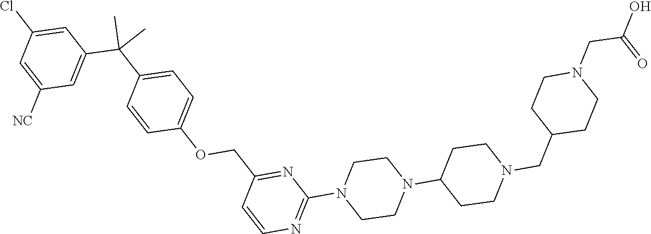 [SMILES: CC(C)(c1ccc(OCc2ccnc(N3CCN(C4CCN(CC5CCN(CC(=O)O)CC5)CC4)CC3)n2)cc1)c1cc(Cl)cc(C#N)c1]

50d (200 mg, 0.27 mmol) was dissolved in 2 mL of DCM, and 2 mL of trifluoroacetic acid was added. The mixture was stirred at room temperature for 12 h. The reaction liquid was concentrated under reduced pressure, to afford crude 50e trifluoroacetate (200 mg).

LCMS m/z=686.3 [M+1]⁺

### Step 6: (2S,4R)-1-((S)-2-(2-(4-((4-(4-(4-((4-(2-(3-chloro-5-cyanophenyl)propan-2-yl)phenoxy)methyl)pyrimidin-2-yl)piperazin-1-yl)piperidin-1-yl)methyl)piperidin-1-yl)acetamido)-3,3-dimethylbutanoyl)-4-hydroxy-N-((S)-1-(4-(4-methylthiazol-5-yl)phenyl)ethyl)pyrrolidine-2-carboxamide (Compound 50) trifluoroacetate

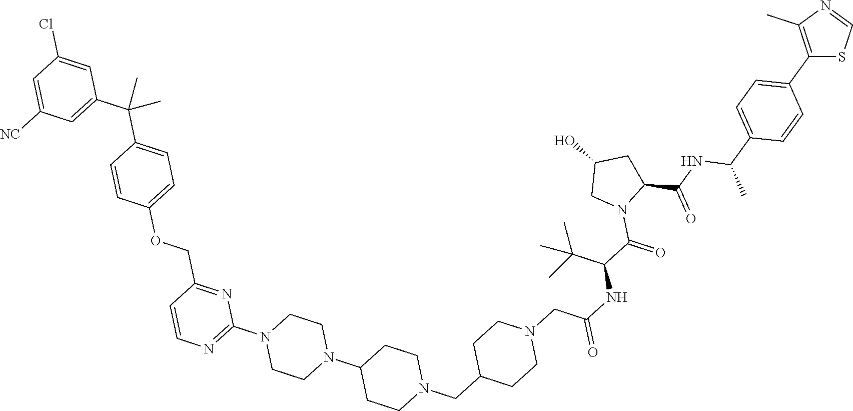 [SMILES: Cc1ncsc1-c1ccc([C@H](C)NC(=O)[C@@H]2C[C@@H](O)CN2C(=O)[C@@H](NC(=O)CN2CCC(CN3CCC(N4CCN(c5nccc(COc6ccc(C(C)(C)c7cc(Cl)cc(C#N)c7)cc6)n5)CC4)CC3)CC2)C(C)(C)C)cc1]

The above crude 50e trifluoroacetate (200 mg) was dissolved in 10 mL of DMF, and DIPEA (250 mg, 1.93 mmol) and HATU (185 mg, 0.49 mmol) were added. The mixture was stirred at room temperature for 30 min, and then (2S,4R)-1-((S)-2-amino-3,3-dimethylbutanoyl)-4-hydroxyl-N-((S)-1-(4-(4-methylthiazol-5-yl)phenyl)ethyl)pyrrolidine-2-carboxamide (see J. Med. Chem. 2019, 62, 1420-1442 for the synthetic method) (170 mg, 0.38 mmol) was added. The resulting mixture was stirred at room temperature for 16 h. To the reaction liquid was added 30 mL of water, and the mixture was extracted with 100 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 50 trifluoroacetate (50 mg).

¹H NMR (400 MHz, CD₃OD) δ 9.03-8.88 (m, 1H), 8.40 (d, 1H), 7.62-7.56 (m, 1H), 7.52-7.33 (m, 6H), 7.20-7.12 (m, 2H), 6.97-6.86 (m, 3H), 5.06-4.96 (m, 3H), 4.68-4.52 (m, 2H), 4.48-3.86 (m, 8H), 3.86-3.55 (m, 6H), 3.54-3.34 (m, 4H), 3.25-3.00 (m, 6H), 2.54-2.40 (m, 5H), 2.35-1.91 (m, 7H), 1.79-1.46 (m, 11H), 1.13-0.98 (m, 9H).

LCMS m/z=371.7 [M/3+1]⁺

### Example 51

### (2S,4R)-1-((S)-2-(2-(4-((4-(4-((4-(2-(3-chloro-5-cyanophenyl)propan-2-yl)phenoxy)methyl)pyrimidin-2-yl)piperazin-1-yl)methyl)piperidin-1-yl)acetamido)-3,3-dimethylbutanoyl)-4-hydroxy-N-((S)-1-(4-(4-methylthiazol-5-yl)phenyl)ethyl)pyrrolidine-2-carboxamide (Compound 51) trifluoroacetate

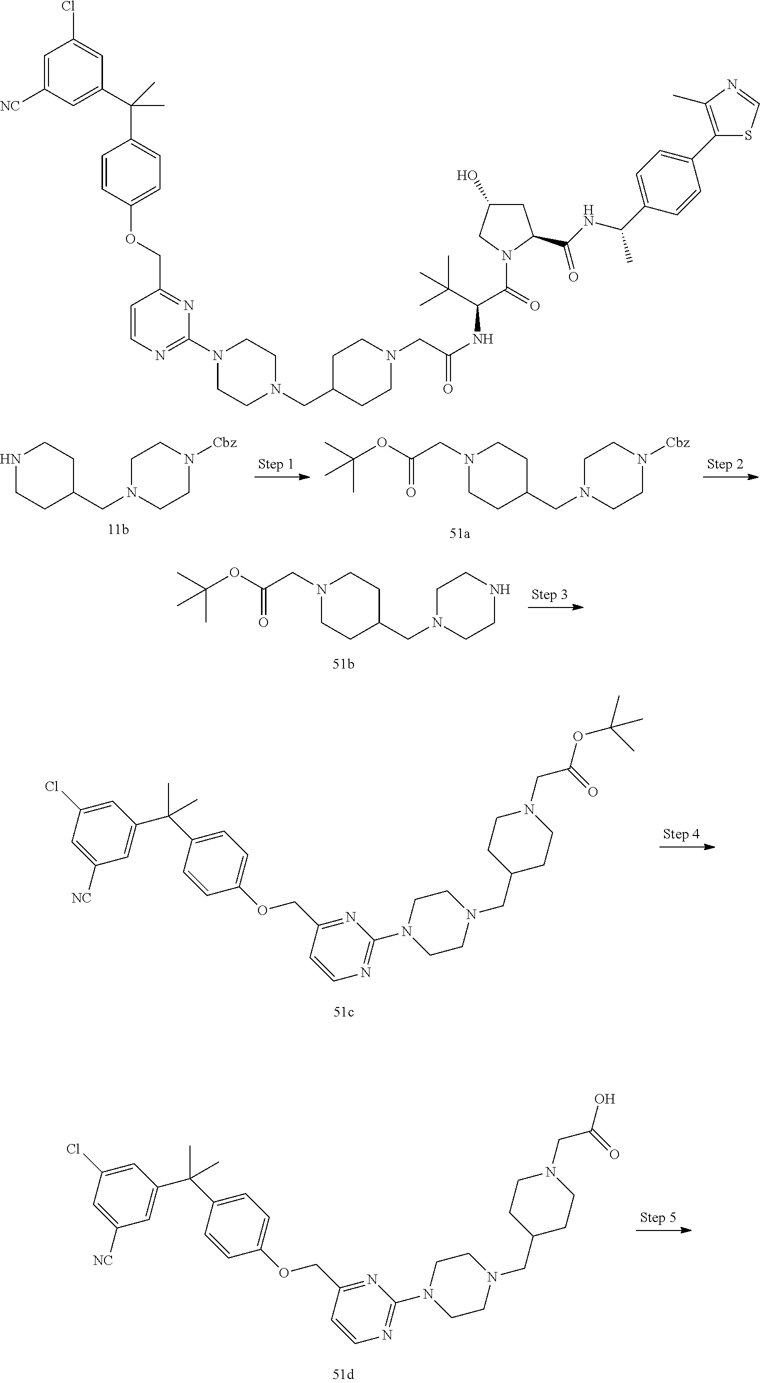

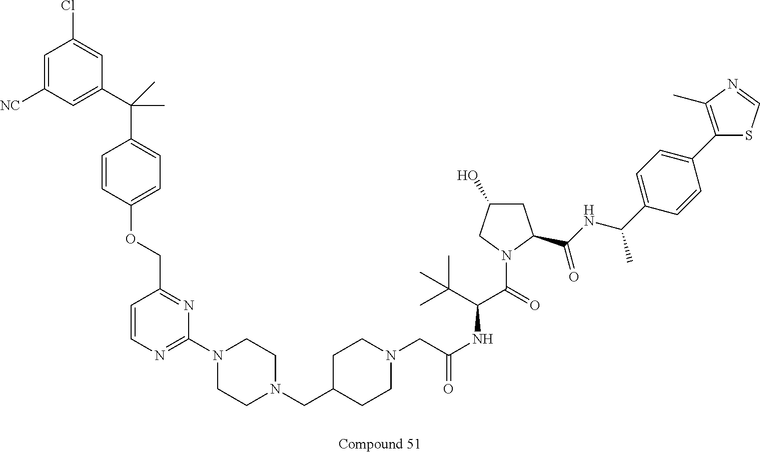 [SMILES: Cc1ncsc1-c1ccc([C@H](C)NC(=O)[C@@H]2C[C@@H](O)CN2C(=O)[C@@H](NC(=O)CN2CCC(CN3CCN(c4nccc(COc5ccc(C(C)(C)c6cc(Cl)cc(C#N)c6)cc5)n4)CC3)CC2)C(C)(C)C)cc1]

### Step 1: benzyl 4-((1-(2-(tert-butoxy)-2-oxoethyl)piperidin-4-yl)methyl)piperazine-1-carboxylate (51a)

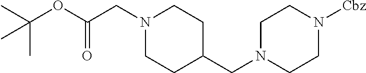 [SMILES: CC(C)(C)OC(=O)CN1CCC(CN2CCN(C(=O)OCc3ccccc3)CC2)CC1]

The above crude 11b (1.0 g) was dissolved in 10 mL of DMF, and solid potassium carbonate (0.69 g, 5.0 mmol) and tert-butyl bromoacetate (0.61 g, 3.13 mmol) were added. The mixture was reacted at room temperature for 12 h. The reaction liquid was added to 50 mL of water, and the mixture was extracted with ethyl acetate (50 mL×3), dried over anhydrous sodium sulfate, and concentrated under reduced pressure. The crude product was separated and purified by silica gel column chromatography (ethyl acetate/petroleum ether (v/v)=1:100-1:1), to afford 51a (0.6 g, two-step yield from compound 11a: 45%).

LCMS m/z=432.3 [M+1]⁺

### Step 2: tert-butyl 2-(4-(piperazin-1-ylmethyl)piperidin-1-yl)acetate (51b)

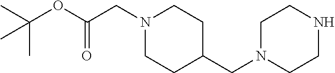 [SMILES: CC(C)(C)OC(=O)CN1CCC(CN2CCNCC2)CC1]

51a (0.6 g, 1.4 mmol) was dissolved in 20 mL of methanol, and 0.2 g of 10% palladium on carbon was added. The mixture was reacted at room temperature under hydrogen atmosphere for 16 h. The reaction system was filtered by suction, and the filtrate was concentrated under reduced pressure, to afford crude 51b (0.41 g).

LCMS m/z=298.3 [M+1]⁺

### Step 3: tert-butyl 2-(4-((4-(4-((4-(2-(3-chloro-5-cyanophenyl)propan-2-yl)phenoxy)methyl)pyrimidin-2-yl)piperazin-1-yl)methyl)piperidin-1-yl)acetate (51c)

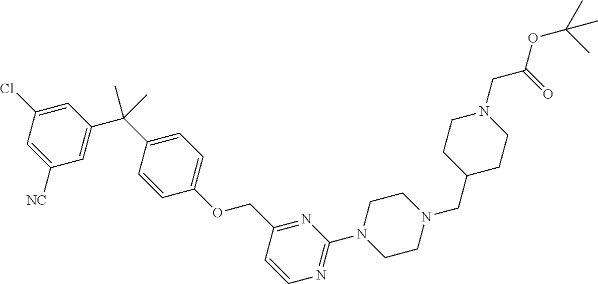 [SMILES: CC(C)(C)OC(=O)CN1CCC(CN2CCN(c3nccc(COc4ccc(C(C)(C)c5cc(Cl)cc(C#N)c5)cc4)n3)CC2)CC1]

3-chloro-5-(2-(4-((2-chloropyrimidin-4-yl)methoxy)phenyl)propan-2-yl)benzonitrile (9d) (0.33 g, 0.83 mmol) was dissolved in 10 mL of DMF, and solid potassium bicarbonate (105 mg, 1.05 mmol) and the above crude 51b (0.25 g) were successively added. The mixture was warmed to 60° C. and reacted for 3 h. The reaction liquid was cooled to room temperature, and 20 mL of water was slowly added. The mixture was extracted with ethyl acetate (50 mL×3), and the organic phase was washed with water (50 mL×3), dried over anhydrous sodium sulfate, and concentrated under reduced pressure. The crude product was separated and purified by silica gel column chromatography (dichloromethane/methanol (v/v)=15:1), to afford 51c (200 mg, yield from compound 9d: 37%).

LCMS m/z=659.2 [M+1]⁺

### Step 4: 2-(4-((4-(4-((4-(2-(3-chloro-5-cyanophenyl)propan-2-yl)phenoxy)methyl)pyrimidin-2-yl)piperazin-1-yl)methyl)piperidin-1-yl)acetic acid (51d) trifluoroacetate

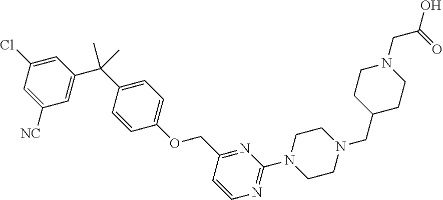 [SMILES: CC(C)(c1ccc(OCc2ccnc(N3CCN(CC4CCN(CC(=O)O)CC4)CC3)n2)cc1)c1cc(Cl)cc(C#N)c1]

51c (200 mg, 0.3 mmol) was dissolved in 2 mL of DCM, and 2 mL of trifluoroacetic acid was added. The mixture was stirred at room temperature for 12 h. The reaction liquid was concentrated under reduced pressure, to afford crude 51d trifluoroacetate (200 mg).

LCMS m/z=603.2 [M+1]⁺

### Step 5: (2S,4R)-1-((S)-2-(2-(4-((4-(4-((4-(2-(3-chloro-5-cyanophenyl)propan-2-yl)phenoxy)methyl)pyrimidin-2-yl)piperazin-1-yl)methyl)piperidin-1-yl)acetamido)-3,3-dimethylbutanoyl)-4-hydroxy-N-((S)-1-(4-(4-methylthiazol-5-yl)phenyl)ethyl)pyrrolidine-2-carboxamide (Compound 51) trifluoroacetate

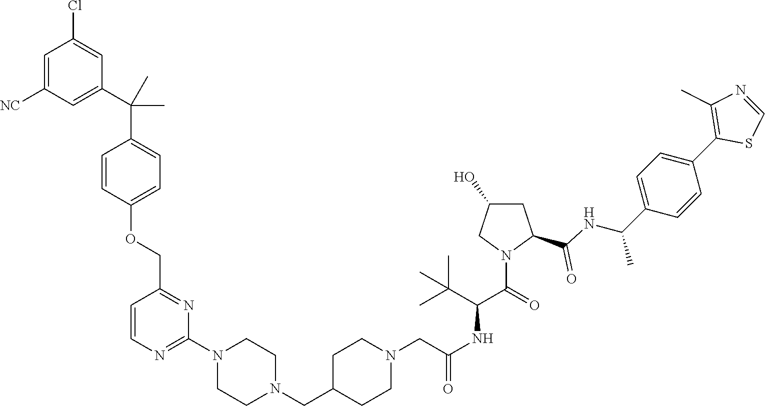 [SMILES: Cc1ncsc1-c1ccc([C@H](C)NC(=O)[C@@H]2C[C@@H](O)CN2C(=O)[C@@H](NC(=O)CN2CCC(CN3CCN(c4nccc(COc5ccc(C(C)(C)c6cc(Cl)cc(C#N)c6)cc5)n4)CC3)CC2)C(C)(C)C)cc1]

The above crude 51d trifluoroacetate (200 mg) was dissolved in 10 mL of DMF, and DIPEA (250 mg, 1.93 mmol) and HATU (185 mg, 0.49 mmol) were added. The mixture was stirred at room temperature for 30 min, and then (2S,4R)-1-((S)-2-amino-3,3-dimethylbutanoyl)-4-hydroxyl-N-((S)-1-(4-(4-methylthiazol-5-yl)phenyl)ethyl)pyrrolidine-2-carboxamide (see J. Med. Chem. 2019, 62, 1420-1442 for the synthetic method) (170 mg, 0.38 mmol) was added. The resulting mixture was stirred at room temperature for 16 h. To the reaction liquid was added 30 mL of water, and the mixture was extracted with 100 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 51 trifluoroacetate (45 mg).

¹H NMR (400 MHz, CD₃OD) δ 9.01-8.92 (m, 1H), 8.45-8.33 (m, 1H), 7.62-7.54 (m, 1H), 7.53-7.34 (m, 6H), 7.20-7.10 (m, 2H), 6.98-6.84 (m, 3H), 5.07-4.97 (m, 3H), 4.69-4.54 (m, 2H), 4.50-4.35 (m, 1H), 4.12-2.90 (m, 18H), 2.53-2.45 (m, 3H), 2.44-2.07 (m, 4H), 2.05-1.90 (m, 1H), 1.84-1.46 (m, 11H), 1.14-0.98 (m, 9H).

LCMS m/z=344.0 [M/3+1]⁺

### Example 52

### 3-chloro-5-(2-(4-((2-(4-(1-(1-(2-(2,6-dioxopiperidin-3-yl)-1,3-dioxoisoindolin-5-yl)piperidin-3-yl)azetidin-3-yl)piperazin-1-yl)pyrimidin-4-yl)methoxy)phenyl)propan-2-yl)benzonitrile (Compound 52) trifluoroacetate

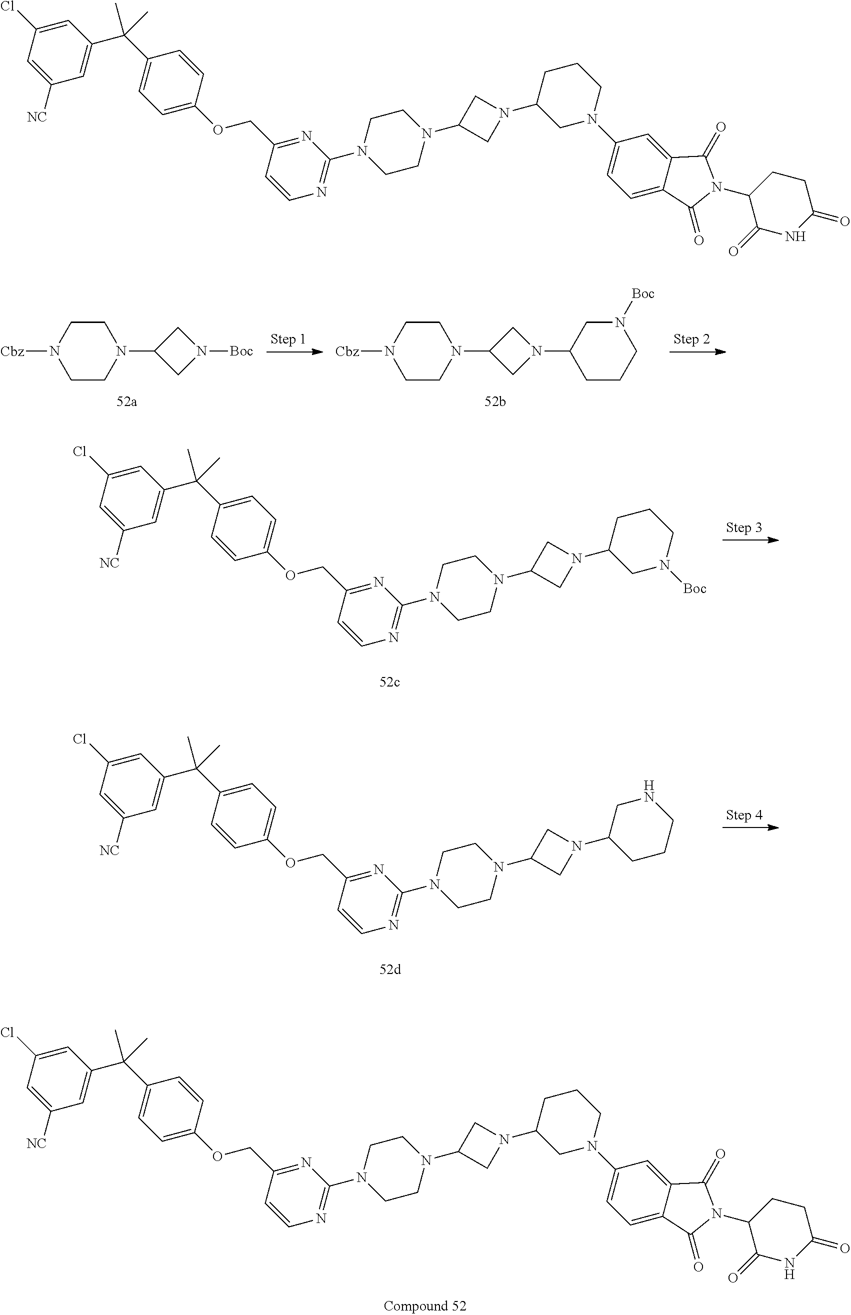

### Step 1: benzyl 4-(1-(1-(tert-butoxycarbonyl)piperidin-3-yl)azetidin-3-yl)piperazine-1-carboxylate (52b)

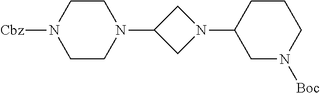 [SMILES: CC(C)(C)OC(=O)N1CCCC(N2CC(N3CCN(C(=O)OCc4ccccc4)CC3)C2)C1]

Benzyl 4-(1-(tert-butoxycarbonyl)azetidine-3-yl)piperazine-1-carboxylate (52a) (see WO 2019195609 for the synthetic method) (310 mg, 0.83 mmol) was dissolved in 4 mL of dichloromethane, and 2 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (210 mg, 2.5 mmol) was added, and the mixture was stirred at room temperature for 30 min. Tert-butyl 3-oxopiperidine-1-carboxylate (170 mg, 0.89 mmol) was then added, and the mixture was stirred at room temperature for 1 h. Sodium triacetoxyborohydride (700 mg, 3.3 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 52b (291 mg, yield: 77%).

LCMS m/z=459.3 [M+1]⁺

### Step 2: tert-butyl 3-(3-(4-(4-((4-(2-(3-chloro-5-cyanophenyl)propan-2-yl)phenoxy)methyl)pyrimidin-2-yl)piperazin-1-yl)azetidin-1-yl)piperidine-1-carboxylate (52c)

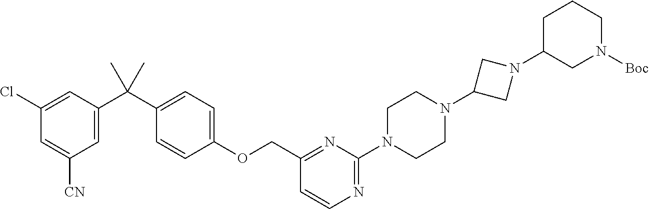 [SMILES: CC(C)(C)OC(=O)N1CCCC(N2CC(N3CCN(c4nccc(COc5ccc(C(C)(C)c6cc(Cl)cc(C#N)c6)cc5)n4)CC3)C2)C1]

52b (0.15 g, 0.33 mmol) was dissolved in 5 mL of methanol, and 15 mg of 10% palladium on carbon was added. The mixture was reacted at room temperature under hydrogen atmosphere for 4 h. The reaction liquid was filtered, the filtrate was concentrated under reduced pressure, and the residue was dissolved with 5 mL of DMF. Solid potassium bicarbonate (62 mg, 0.62 mmol) and 3-chloro-5-(2-(4-((2-chloropyrimidin-4-yl)methoxy)phenyl)propan-2-yl)benzonitrile (9d) (120 mg, 0.30 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 52c (64 mg, yield from compound 9d: 31%).

LCMS m/z=686.3 [M+1]⁺

### Step 3: 3-chloro-5-(2-(4-((2-(4-(1-(piperidin-3-yl)azetidin-3-yl)piperazin-1-yl)pyrimidin-4-yl)methoxy)phenyl)propan-2-yl)benzonitrile (52d) trifluoroacetate

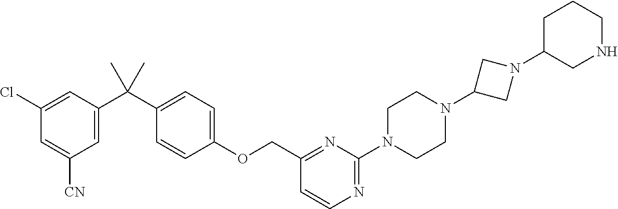 [SMILES: CC(C)(c1ccc(OCc2ccnc(N3CCN(C4CN(C5CCCNC5)C4)CC3)n2)cc1)c1cc(Cl)cc(C#N)c1]

52c (96 mg, 0.14 mmol) was dissolved in 4 mL of dichloromethane, and 2 mL of trifluoroacetic acid was added. The mixture was stirred at room temperature for 4 h. The reaction liquid was concentrated under reduced pressure, to afford crude 52d trifluoroacetate (110 mg).

### Step 4: 3-chloro-5-(2-(4-((2-(4-(1-(1-(2-(2,6-dioxopiperidin-3-yl)-1,3-dioxoisoindolin-5-yl)piperidin-3-yl)azetidin-3-yl)piperazin-1-yl)pyrimidin-4-yl)methoxy)phenyl)propan-2-yl)benzonitrile (Compound 52) trifluoroacetate

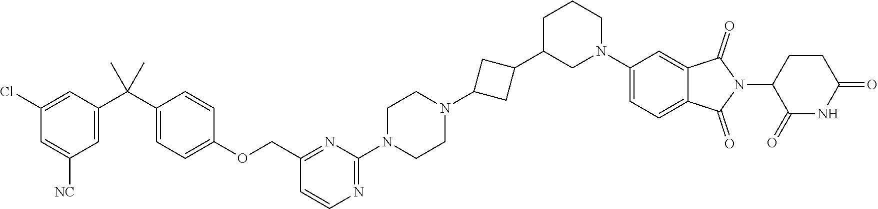 [SMILES: CC(C)(c1ccc(OCc2ccnc(N3CCN(C4CC(C5CCCN(c6ccc7c(c6)C(=O)N(C6CCC(=O)NC6=O)C7=O)C5)C4)CC3)n2)cc1)c1cc(Cl)cc(C#N)c1]

The above crude 52d trifluoroacetate (110 mg) was dissolved in 11 mL of DMSO, and solid sodium bicarbonate (59 mg, 0.70 mmol), 0.15 mL of DIPEA and 2-(2,6-dioxopiperidin-3-yl)-5-fluoroisoindoline-1,3-dione (see WO 2017197056 for the synthetic method) (46 mg, 0.17 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 52 trifluoroacetate (39 mg).

¹H NMR (400 MHz, CDCl₃) δ 8.90 (br.s, 1H), 8.36 (d, 1H), 7.66 (d, 1H), 7.48-7.26 (m, 4H), 7.14-7.02 (m, 3H), 6.93-6.82 (m, 3H), 5.02-4.90 (m, 3H), 4.66-4.46 (m, 4H), 4.28-4.04 (m, 5H), 3.94-3.81 (m, 1H), 3.71-3.58 (m, 1H), 3.50-3.38 (m, 1H), 3.22-2.65 (m, 9H), 2.20-1.90 (m, 3H), 1.75-1.55 (m, 8H).

LCMS m/z=842.5 [M+1]⁺.

### Example 53

### 3-chloro-5-(2-(4-((2-(4-(1-(7-(2-(2,6-dioxopiperidin-3-yl)-1,3-dioxoisoindolin-5-yl)-7-azaspiro[3.5]nonan-2-yl)azetidin-3-yl)piperazin-1-yl)pyrimidin-4-yl)methoxy)phenyl) propan-2-yl)benzonitrile (Compound 53) trifluoroacetate

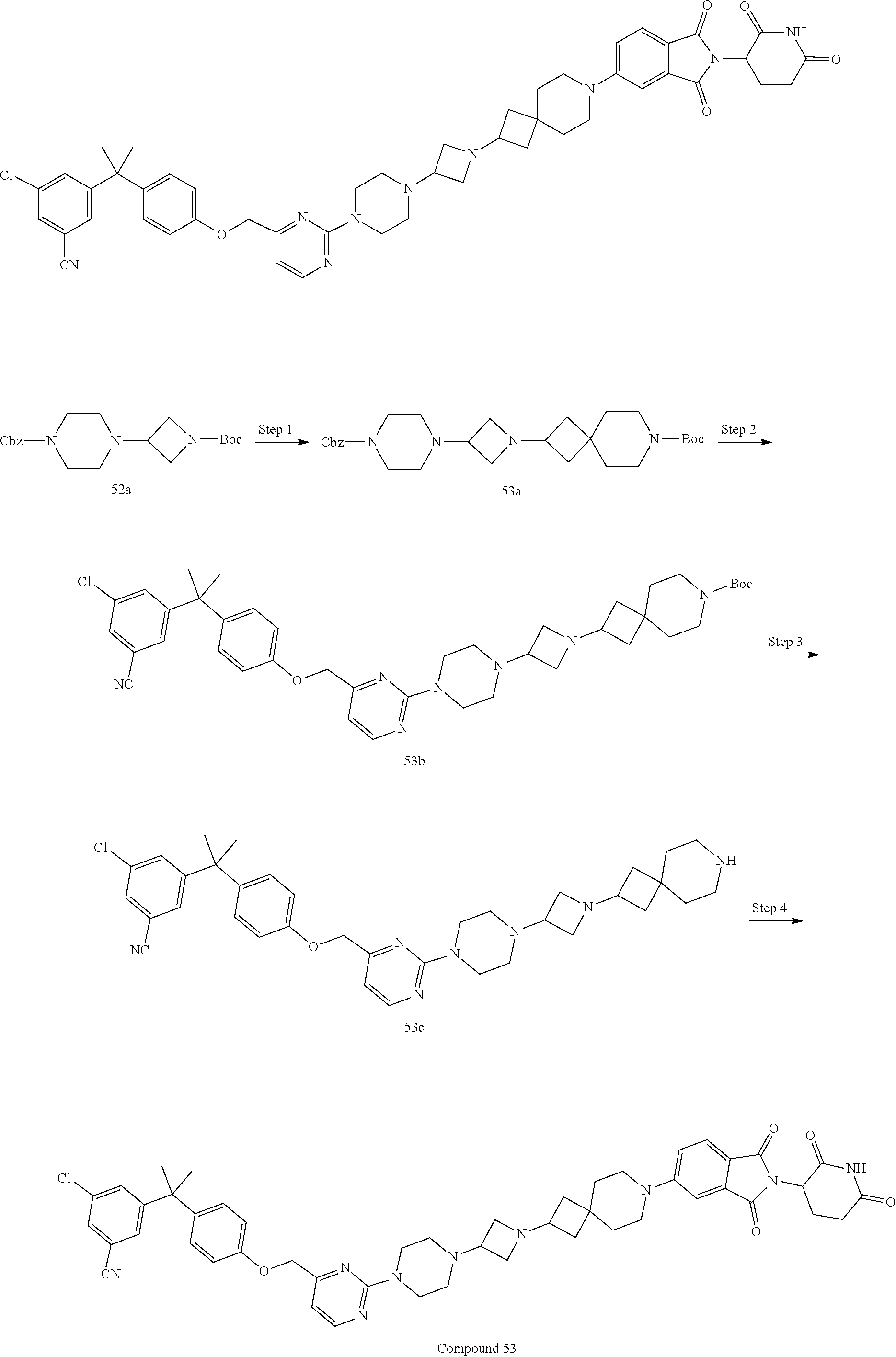

### Step 1: tert-butyl 2-(3-(4-((benzyloxy)carbonyl)piperazin-1-yl)azetidin-1-yl)-7-azaspiro[3.5]nonane-7-carboxylate (53a)

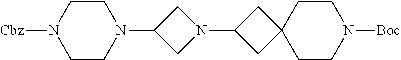 [SMILES: CC(C)(C)OC(=O)N1CCC2(CC1)CC(N1CC(N3CCN(C(=O)OCc4ccccc4)CC3)C1)C2]

52a (see WO 2019195609 for the synthetic method) (310 mg, 0.83 mmol) was dissolved in 4 mL of dichloromethane, and 2 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (210 mg, 2.5 mmol) was added, and the mixture was stirred at room temperature for 30 min. Tert-butyl 2-oxo-7-azaspiro[3.5]nonane-7-carboxylate (400 mg, 1.67 mmol) was then added, and the mixture was stirred at room temperature for 1 h. Sodium triacetoxyborohydride (700 mg, 3.3 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 53a (301 mg, yield: 73%).

LCMS m/z=499.3 [M+1]⁺

### Step 2: tert-butyl 2-(3-(4-(4-((4-(2-(3-chloro-5-cyanophenyl)propan-2-yl)phenoxy)methyl)pyrimidin-2-yl)piperazin-1-yl)azetidin-1-yl)-7-azaspiro[3.5]nonane-7-carboxylate (53b)

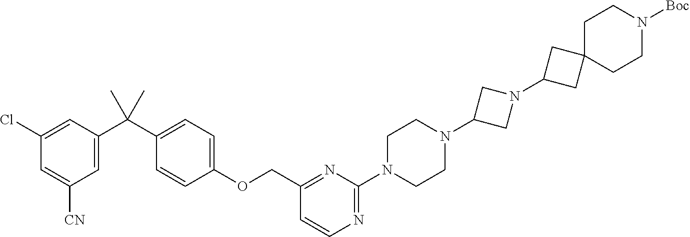 [SMILES: CC(C)(C)OC(=O)N1CCC2(CC1)CC(N1CC(N3CCN(c4nccc(COc5ccc(C(C)(C)c6cc(Cl)cc(C#N)c6)cc5)n4)CC3)C1)C2]

53a (0.13 g, 0.26 mmol) was dissolved in 5 mL of methanol, and 13 mg of 10% palladium on carbon was added. The mixture was reacted at room temperature under hydrogen atmosphere for 4 h. The reaction liquid was filtered, the filtrate was concentrated under reduced pressure, and the residue was dissolved with 5 mL of DMF. Solid potassium bicarbonate (51 mg, 0.51 mmol) and 3-chloro-5-(2-(4-((2-chloropyrimidin-4-yl)methoxy)phenyl)propan-2-yl)benzonitrile (9d) (100 mg, 0.25 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 53b (51 mg, yield: 27%).

LCMS m/z=726.3 [M+1]⁺

### Step 3: 3-(2-(4-((2-(4-(1-(7-azaspiro[3.5]nonan-2-yl)azetidin-3-yl)piperazin-1-yl)pyrimidin-4-yl)methoxy)phenyl)propan-2-yl)-5-chlorobenzonitrile (53c) trifluoroacetate

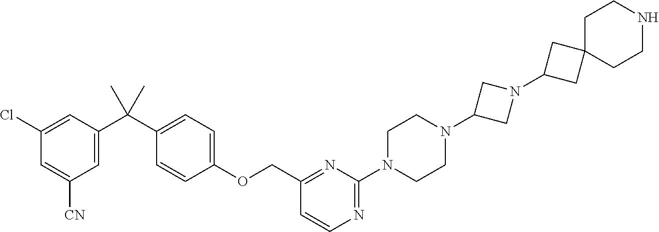 [SMILES: CC(C)(c1ccc(OCc2ccnc(N3CCN(C4CN(C5CC6(CCNCC6)C5)C4)CC3)n2)cc1)c1cc(Cl)cc(C#N)c1]

53b (50 mg, 0.07 mmol) was dissolved in 4 mL of dichloromethane, and 2 mL of trifluoroacetic acid was added. The mixture was stirred at room temperature for 4 h. The reaction liquid was concentrated under reduced pressure, to afford crude 53c trifluoroacetate (66 mg).

### Step 4: 3-chloro-5-(2-(4-((2-(4-(1-(7-(2-(2,6-dioxopiperidin-3-yl)-1,3-dioxoisoindolin-5-yl)-7-azaspiro[3.5]nonan-2-yl)azetidin-3-yl)piperazin-1-yl)pyrimidin-4-yl)methoxy)phenyl) propan-2-yl)benzonitrile (Compound 53) trifluoroacetate

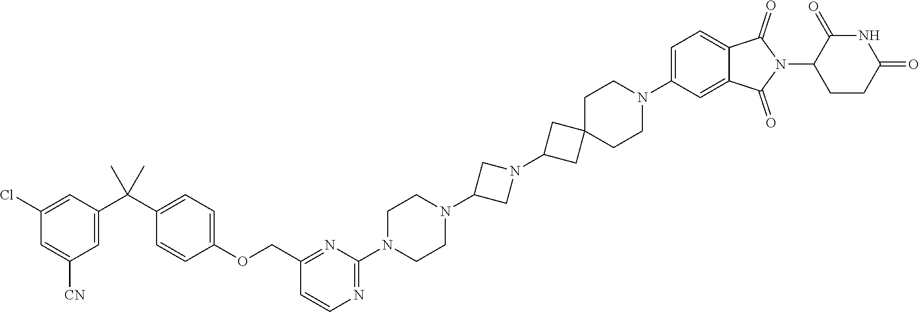 [SMILES: CC(C)(c1ccc(OCc2ccnc(N3CCN(C4CN(C5CC6(CCN(c7ccc8c(c7)C(=O)N(C7CCC(=O)NC7=O)C8=O)CC6)C5)C4)CC3)n2)cc1)c1cc(Cl)cc(C#N)c1]

The above crude 53c trifluoroacetate (66 mg) was dissolved in 5 mL of DMSO, and solid sodium bicarbonate (29 mg, 0.35 mmol), 0.10 mL of DIPEA and 2-(2,6-dioxopiperidin-3-yl)-5-fluoroisoindoline-1,3-dione (see WO 2017197056 for the synthetic method) (23 mg, 0.083 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 53 trifluoroacetate (25 mg).

¹H NMR (400 MHz, CDCl₃) δ 8.38 (d, 1H), 8.01 (br.s, 1H), 7.68 (d, 1H), 7.47-7.42 (m, 2H), 7.36-7.32 (m, 1H), 7.29-7.26 (m, 1H), 7.15-7.00 (m, 3H), 6.93-6.83 (m, 3H), 5.00-4.88 (m, 3H), 4.60-3.95 (m, 9H), 3.45-3.28 (m, 4H), 3.10-2.65 (m, 8H), 2.34-2.22 (m, 2H), 2.18-1.99 (m, 3H), 1.81-1.70 (m, 4H), 1.65 (s, 6H).

LCMS m/z=882.7 [M+1]⁺.

### Example 54: 3-chloro-5-(2-(4-((2-(4-(1-(1-(2-(2,6-dioxopiperidin-3-yl)-1,3-dioxoisoindolin-5-yl)piperidin-4-yl)azetidin-3-yl)piperazin-1-yl)pyrimidin-4-yl)methoxy)phenyl)propan-2-yl)benzonitrile (Compound 54)

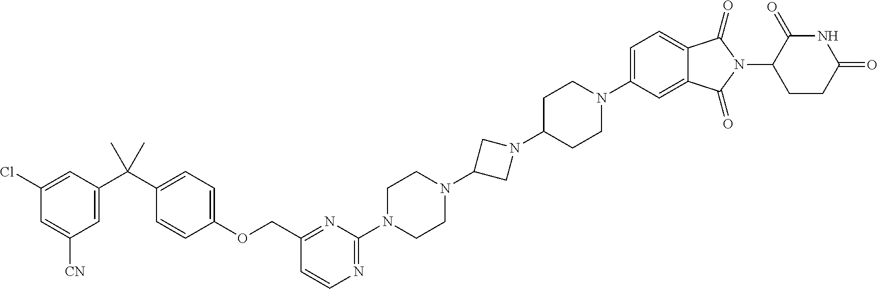 [SMILES: CC(C)(c1ccc(OCc2ccnc(N3CCN(C4CN(C5CCN(c6ccc7c(c6)C(=O)N(C6CCC(=O)NC6=O)C7=O)CC5)C4)CC3)n2)cc1)c1cc(Cl)cc(C#N)c1]

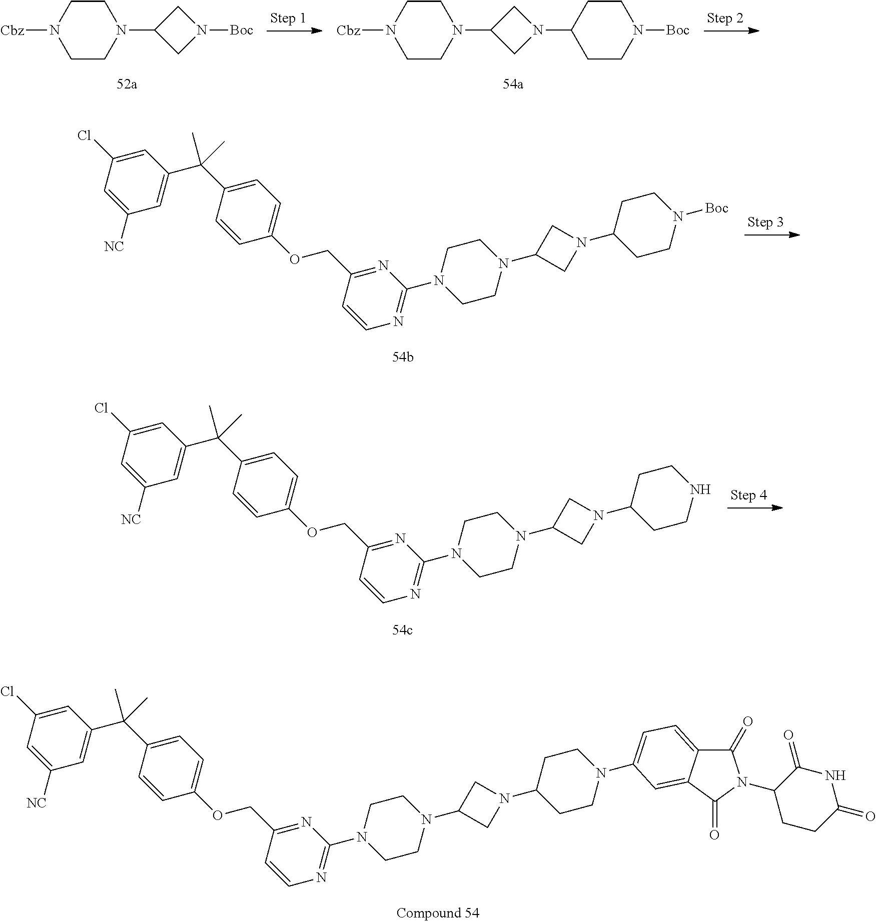 [SMILES: CC(C)(C)OC(=O)N1CC(N2CCN(C(=O)OCc3ccccc3)CC2)C1 ; CC(C)(C)OC(=O)N1CCC(N2CC(N3CCN(C(=O)OCc4ccccc4)CC3)C2)CC1 ; CC(C)(C)OC(=O)N1CCC(N2CC(N3CCN(c4nccc(COc5ccc(C(C)(C)c6cc(Cl)cc(C#N)c6)cc5)n4)CC3)C2)CC1 ; CC(C)(c1ccc(OCc2ccnc(N3CCN(C4CN(C5CCNCC5)C4)CC3)n2)cc1)c1cc(Cl)cc(C#N)c1 ; CC(C)(c1ccc(OCc2ccnc(N3CCN(C4CN(C5CCN(c6ccc7c(c6)C(=O)N(C6CCC(=O)NC6=O)C7=O)CC5)C4)CC3)n2)cc1)c1cc(Cl)cc(C#N)c1]

### Step 1: benzyl 4-(1-(1-(tert-butoxycarbonyl)piperidin-4-yl)azetidin-3-yl)piperazine-1-carboxylate (54a)

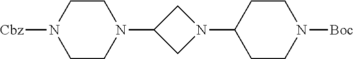 [SMILES: CC(C)(C)OC(=O)N1CCC(N2CC(N3CCN(C(=O)OCc4ccccc4)CC3)C2)CC1]

Benzyl 4-(1-(tert-butoxycarbonyl)azetidine-3-yl)piperazine-1-carboxylate (52a) (see WO 2019195609 for the synthetic method) (110 mg, 0.29 mmol) was dissolved in 2 mL of dichloromethane, and 1 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (71 mg, 0.84 mmol) was added, and the mixture was stirred at room temperature for 30 min. Tert-butyl 4-oxopiperidine-1-carboxylate (110 mg, 0.55 mmol) was then added, and the mixture was stirred at room temperature for 1 h. Sodium triacetoxyborohydride (240 mg, 1.13 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 54a (78 mg, yield: 59%).

LCMS m/z=459.3 [M+1]⁺

### Step 2: tert-butyl 4-(3-(4-(4-((4-(2-(3-chloro-5-cyanophenyl)propan-2-yl)phenoxy)methyl)pyrimidin-2-yl)piperazin-1-yl)azetidin-1-yl)piperidine-1-carboxylate (54b)

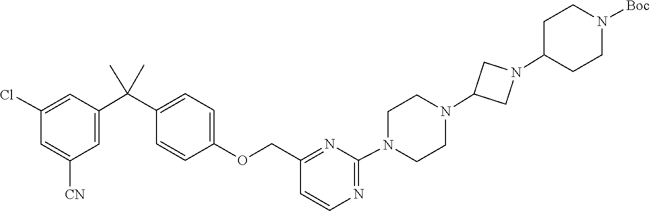 [SMILES: CC(C)(C)OC(=O)N1CCC(N2CC(N3CCN(c4nccc(COc5ccc(C(C)(C)c6cc(Cl)cc(C#N)c6)cc5)n4)CC3)C2)CC1]

54a (0.12 g, 0.26 mmol) was dissolved in 5 mL of methanol, and 20 mg of 10% palladium on carbon was added. The mixture was reacted at room temperature under hydrogen atmosphere for 4 h. The reaction liquid was filtered, the filtrate was concentrated under reduced pressure, and the residue was dissolved with 5 mL of DMF. Solid potassium bicarbonate (54 mg, 0.54 mmol) and 3-chloro-5-(2-(4-((2-chloropyrimidin-4-yl)methoxy)phenyl)propan-2-yl)benzonitrile (9d) (110 mg, 0.28 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 54b (82 mg, yield from compound 9d: 43%).

LCMS m/z=686.3 [M+1]⁺

### Step 3: 3-chloro-5-(2-(4-((2-(4-(1-(piperidin-4-yl)azetidin-3-yl)piperazin-1-yl)pyrimidin-4-yl)methoxy)phenyl)propan-2-yl)benzonitrile (54c) trifluoroacetate

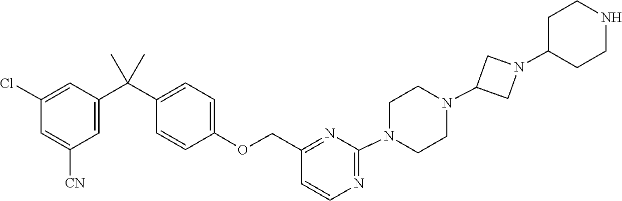 [SMILES: CC(C)(c1ccc(OCc2ccnc(N3CCN(C4CN(C5CCNCC5)C4)CC3)n2)cc1)c1cc(Cl)cc(C#N)c1]

54b (67 mg, 0.098 mmol) was dissolved in 3 mL of dichloromethane, and 1.5 mL of trifluoroacetic acid was added. The mixture was stirred at room temperature for 4 h. The reaction liquid was concentrated under reduced pressure, to afford crude 54c trifluoroacetate (78 mg).

Step 4: 3-chloro-5-(2-(4-((2-(4-(1-(1-(2-(2,6-dioxopiperidin-3-yl)-1,3-dioxoisoindolin-5-yl)piperidin-4-yl)azetidin-3-yl)piperazin-1-yl)pyrimidin-4-yl)methoxy)phenyl)propan-2-yl)benzonitrile (Compound 54)

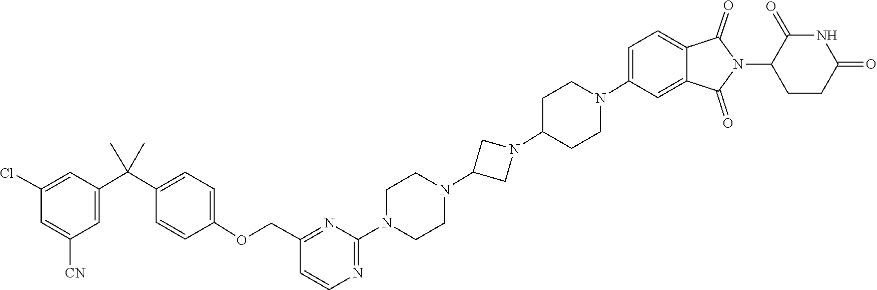 [SMILES: CC(C)(c1ccc(OCc2ccnc(N3CCN(C4CN(C5CCN(c6ccc7c(c6)C(=O)N(C6CCC(=O)NC6=O)C7=O)CC5)C4)CC3)n2)cc1)c1cc(Cl)cc(C#N)c1]

The above crude 54c trifluoroacetate (78 mg) was dissolved in 6 mL of DMSO, and solid sodium bicarbonate (41 mg, 0.49 mmol), 0.1 mL of DIPEA and 2-(2,6-dioxopiperidin-3-yl)-5-fluoroisoindoline-1,3-dione (see WO 2017197056 for the synthetic method) (32 mg, 0.12 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure. The crude product was separated and purified by silica gel column chromatography (dichloromethane/methanol (v/v)=1:0-9:1), to afford compound 54 (21 mg, two-step yield from compound 54b: 25%).

¹H NMR (400 MHz, CDCl₃) δ 8.31 (d, 1H), 8.16-8.09 (m, 1H), 7.66 (d, 1H), 7.46-7.40 (m, 2H), 7.39-7.34 (m, 1H), 7.30-7.26 (m, 1H), 7.14-7.00 (m, 3H), 6.93-6.83 (m, 2H), 6.72 (d, 1H), 4.98-4.88 (m, 3H), 3.92-3.72 (m, 6H), 3.66-3.44 (m, 2H), 3.17-2.65 (m, 8H), 2.48-2.30 (m, 5H), 2.20-2.06 (m, 1H), 1.91-1.76 (m, 2H), 1.65 (s, 6H), 1.53-1.38 (m, 2H).

LCMS m/z=842.3 [M+1]⁺.

### Example 55

### 3-chloro-5-(2-(4-((2-(4-(1-(1-(2-(2,6-dioxopiperidin-3-yl)-1,3-dioxoisoindolin-5-yl)pyrrolidin-3-yl)azetidin-3-yl)piperazin-1-yl)pyrimidin-4-yl)methoxy)phenyl)propan-2-yl)benzonitrile (Compound 55) trifluoroacetate

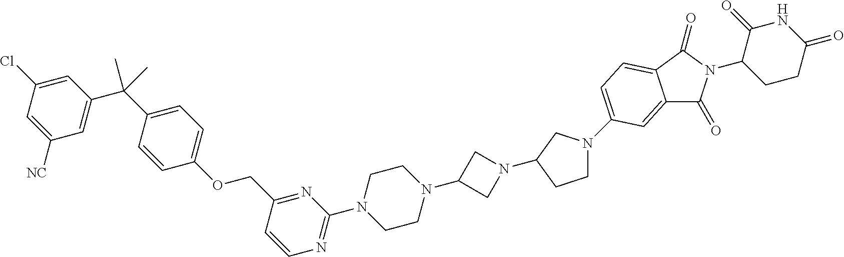 [SMILES: CC(C)(c1ccc(OCc2ccnc(N3CCN(C4CN(C5CCN(c6ccc7c(c6)C(=O)N(C6CCC(=O)NC6=O)C7=O)C5)C4)CC3)n2)cc1)c1cc(Cl)cc(C#N)c1]

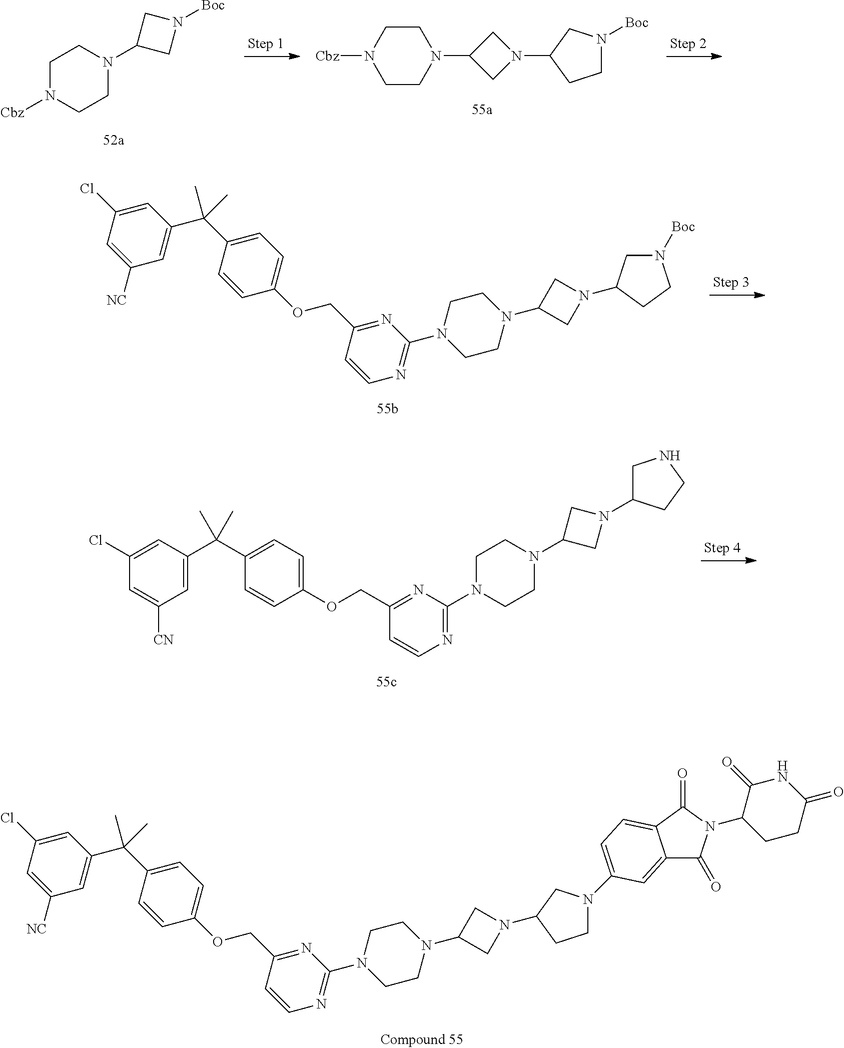 [SMILES: CC(C)(C)OC(=O)N1CC(N2CCN(C(=O)OCc3ccccc3)CC2)C1 ; CC(C)(C)OC(=O)N1CCC(N2CC(N3CCN(C(=O)OCc4ccccc4)CC3)C2)C1 ; CC(C)(C)OC(=O)N1CCC(N2CC(N3CCN(c4nccc(COc5ccc(C(C)(C)c6cc(Cl)cc(C#N)c6)cc5)n4)CC3)C2)C1 ; CC(C)(c1ccc(OCc2ccnc(N3CCN(C4CN(C5CCNC5)C4)CC3)n2)cc1)c1cc(Cl)cc(C#N)c1 ; CC(C)(c1ccc(OCc2ccnc(N3CCN(C4CN(C5CCN(c6ccc7c(c6)C(=O)N(C6CCC(=O)NC6=O)C7=O)C5)C4)CC3)n2)cc1)c1cc(Cl)cc(C#N)c1]

### Step 1: benzyl 4-(1-(1-(tert-butoxycarbonyl)pyrrolidin-3-yl)azetidin-3-yl)piperazine-1-carboxylate (55a)

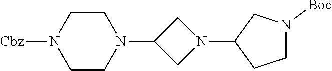 [SMILES: CC(C)(C)OC(=O)N1CCC(N2CC(N3CCN(C(=O)OCc4ccccc4)CC3)C2)C1]

Benzyl 4-(1-(tert-butoxycarbonyl)azetidine-3-yl)piperazine-1-carboxylate (52a) (see WO 2019195609 for the synthetic method) (310 mg, 0.83 mmol) was dissolved in 4 mL of dichloromethane, and 2 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (210 mg, 2.5 mmol) was added, and the mixture was stirred at room temperature for 30 min. Tert-butyl 3-oxopyrrolidine-1-carboxylate (310 mg, 1.67 mmol) was then added, and the mixture was stirred at room temperature for 1 h. Sodium triacetoxyborohydride (700 mg, 3.3 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 55a (321 mg, yield: 87%).

LCMS m/z=445.3 [M+1]⁺

### Step 2: tert-butyl 3-(3-(4-(4-((4-(2-(3-chloro-5-cyanophenyl)propan-2-yl)phenoxy)methyl)pyrimidin-2-yl)piperazin-1-yl)azetidin-1-yl)pyrrolidine-1-carboxylate (55b)

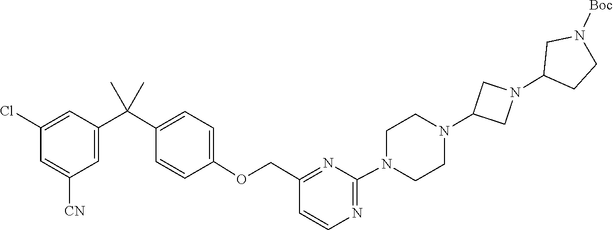 [SMILES: CC(C)(C)OC(=O)N1CCC(N2CC(N3CCN(c4nccc(COc5ccc(C(C)(C)c6cc(Cl)cc(C#N)c6)cc5)n4)CC3)C2)C1]

55a (0.143 g, 0.32 mmol) was dissolved in 5 mL of methanol, and 20 mg of 10% palladium on carbon was added. The mixture was reacted at room temperature under hydrogen atmosphere for 4 h. The reaction liquid was filtered, the filtrate was concentrated under reduced pressure, and the residue was dissolved with 5 mL of DMF. Solid potassium bicarbonate (64 mg, 0.64 mmol) and 3-chloro-5-(2-(4-((2-chloropyrimidin-4-yl)methoxy)phenyl)propan-2-yl)benzonitrile (9d) (130 mg, 0.33 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 55b (101 mg, yield from compound 9d: 46%).

LCMS m/z=672.3 [M+1]⁺

### Step 3: 3-chloro-5-(2-(4-((2-(4-(1-(pyrrolidin-3-yl)azetidin-3-yl)piperazin-1-yl)pyrimidin-4-yl)methoxy)phenyl)propan-2-yl)benzonitrile (55c) trifluoroacetate

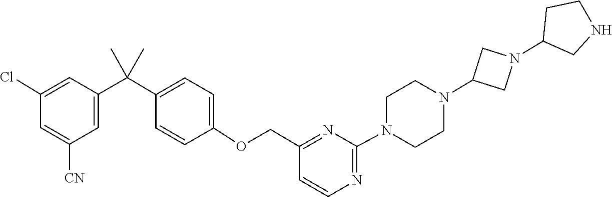 [SMILES: CC(C)(c1ccc(OCc2ccnc(N3CCN(C4CN(C5CCNC5)C4)CC3)n2)cc1)c1cc(Cl)cc(C#N)c1]

55b (100 mg, 0.15 mmol) was dissolved in 3 mL of dichloromethane, and 1.5 mL of trifluoroacetic acid was added. The mixture was stirred at room temperature for 4 h. The reaction liquid was concentrated under reduced pressure, to afford crude 55c trifluoroacetate (118 mg).

Step 4: 3-chloro-5-(2-(4-((2-(4-(1-(1-(2-(2,6-dioxopiperidin-3-yl)-1,3-dioxoisoindolin-5-yl)pyrrolidin-3-yl)azetidin-3-yl)piperazin-1-yl)pyrimidin-4-yl)methoxy)phenyl)propan-2-yl)benzonitrile (Compound 55) trifluoroacetate

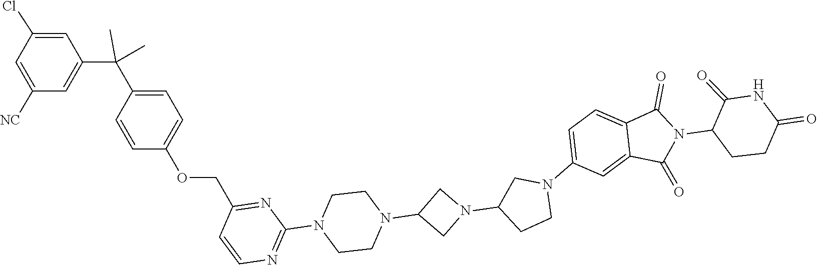 [SMILES: CC(C)(c1ccc(OCc2ccnc(N3CCN(C4CN(C5CCN(c6ccc7c(c6)C(=O)N(C6CCC(=O)NC6=O)C7=O)C5)C4)CC3)n2)cc1)c1cc(Cl)cc(C#N)c1]

The above crude 55c trifluoroacetate (118 mg) was dissolved in 10 mL of DMSO, and solid sodium bicarbonate (63 mg, 0.75 mmol), 0.15 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 55 trifluoroacetate (36 mg).

¹H NMR (400 MHz, CDCl₃) δ 8.47-8.32 (m, 2H), 7.68 (d, 1H), 7.48-7.42 (m, 2H), 7.36-7.31 (m, 1H), 7.16-7.04 (m, 2H), 6.98-6.82 (m, 4H), 6.75-6.66 (m, 1H), 5.01-4.89 (m, 3H), 4.70-4.47 (m, 4H), 4.36-4.05 (m, 6H), 3.80-3.64 (m, 2H), 3.60-3.44 (m, 2H), 3.14-2.96 (m, 4H), 2.96-2.66 (m, 3H), 2.51-2.36 (m, 1H), 2.30-2.10 (m, 2H), 1.65 (s, 6H).

LCMS m/z=828.3 [M+1]⁺.

### Example 56

### 3-chloro-5-(2-(4-((2-(4-(1-((1-(2-(2,6-dioxopiperidin-3-yl)-1,3-dioxoisoindolin-5-yl)piperidin-4-yl)methyl)azetidin-3-yl)piperazin-1-yl)pyrimidin-4-yl)methoxy)phenyl) propan-2-yl)benzonitrile (Compound 56) trifluoroacetate

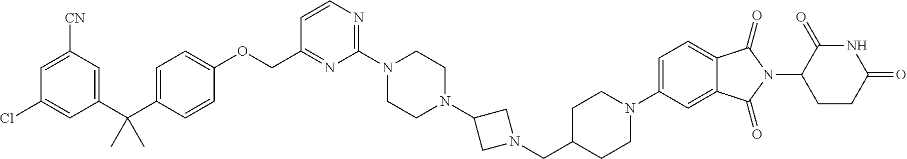 [SMILES: CC(C)(c1ccc(OCc2ccnc(N3CCN(C4CN(CC5CCN(c6ccc7c(c6)C(=O)N(C6CCC(=O)NC6=O)C7=O)CC5)C4)CC3)n2)cc1)c1cc(Cl)cc(C#N)c1]

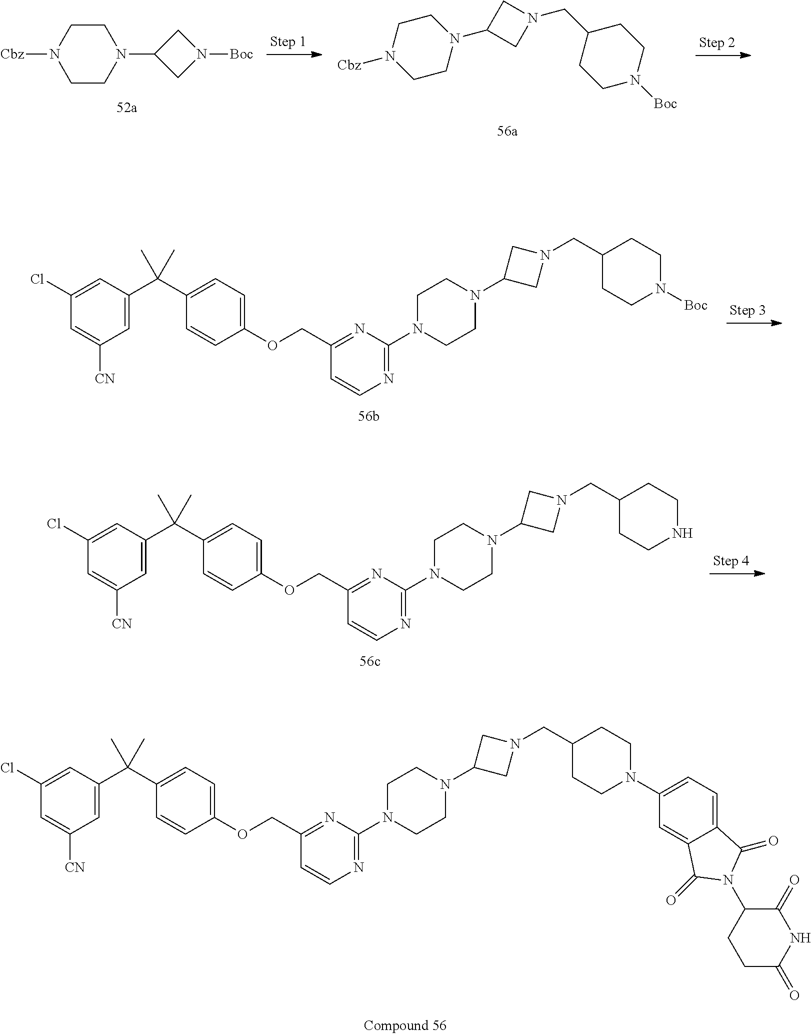 [SMILES: CC(C)(C)OC(=O)N1CC(N2CCN(C(=O)OCc3ccccc3)CC2)C1 ; CC(C)(C)OC(=O)N1CCC(CN2CC(N3CCN(C(=O)OCc4ccccc4)CC3)C2)CC1 ; CC(C)(C)OC(=O)N1CCC(CN2CC(N3CCN(c4nccc(COc5ccc(C(C)(C)c6cc(Cl)cc(C#N)c6)cc5)n4)CC3)C2)CC1 ; CC(C)(c1ccc(OCc2ccnc(N3CCN(C4CN(CC5CCNCC5)C4)CC3)n2)cc1)c1cc(Cl)cc(C#N)c1 ; CC(C)(c1ccc(OCc2ccnc(N3CCN(C4CN(CC5CCN(c6ccc7c(c6)C(=O)N(C6CCC(=O)NC6=O)C7=O)CC5)C4)CC3)n2)cc1)c1cc(Cl)cc(C#N)c1]

### Step 1: benzyl 4-(1-((1-(tert-butoxycarbonyl)piperidin-4-yl)methyl)azetidin-3-yl)piperazine-1-carboxylate

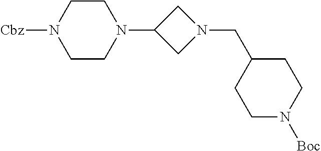 [SMILES: CC(C)(C)OC(=O)N1CCC(CN2CC(N3CCN(C(=O)OCc4ccccc4)CC3)C2)CC1]

Benzyl 4-(1-(tert-butoxycarbonyl)azetidine-3-yl)piperazine-1-carboxylate (52a) (see WO 2019195609 for the synthetic method) (110 mg, 0.29 mmol) was dissolved in 2 mL of dichloromethane, and 1 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (71 mg, 0.85 mmol) was added, and the mixture was stirred at room temperature for 30 min. Tert-butyl 4-formylpiperidine-1-carboxylate (60 mg, 0.28 mmol) was then added, and the mixture was stirred at room temperature for 1 h. Sodium triacetoxyborohydride (240 mg, 1.13 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 56a (88 mg, yield: 64%).

LCMS m/z=473.4 [M+1]⁺

### Step 2: tert-butyl 4-((3-(4-(4-((4-(2-(3-chloro-5-cyanophenyl)propan-2-yl)phenoxy)methyl)pyrimidin-2-yl)piperazin-1-yl)azetidin-1-yl)methyl)piperidine-1-carboxylate (56b)

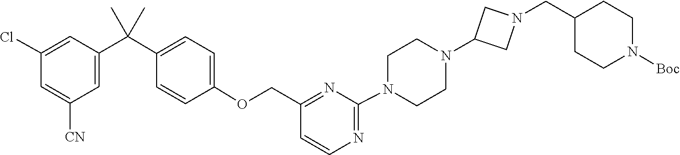 [SMILES: CC(C)(C)OC(=O)N1CCC(CN2CC(N3CCN(c4nccc(COc5ccc(C(C)(C)c6cc(Cl)cc(C#N)c6)cc5)n4)CC3)C2)CC1]

56a (0.200 g, 0.42 mmol) was dissolved in 5 mL of methanol, and 20 mg of 10% palladium on carbon was added. The mixture was reacted at room temperature under hydrogen atmosphere for 4 h. The reaction liquid was filtered, the filtrate was concentrated under reduced pressure, and the residue was dissolved with 5 mL of DMF. Solid potassium bicarbonate (84 mg, 0.84 mmol) and 3-chloro-5-(2-(4-((2-chloropyrimidin-4-yl)methoxy)phenyl)propan-2-yl)benzonitrile (9d) (170 mg, 0.43 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 56b (98 mg, yield from compound 9d: 33%).

LCMS m/z=700.3 [M+1]⁺

### Step 3: 3-chloro-5-(2-(4-((2-(4-(1-(piperidin-4-ylmethyl)azetidin-3-yl)piperazin-1-yl)pyrimidin-4-yl)methoxy)phenyl)propan-2-yl)benzonitrile (56c) trifluoroacetate

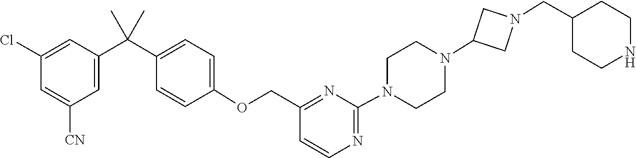 [SMILES: CC(C)(c1ccc(OCc2ccnc(N3CCN(C4CN(CC5CCNCC5)C4)CC3)n2)cc1)c1cc(Cl)cc(C#N)c1]

56b (91 mg, 0.13 mmol) was dissolved in 3 mL of dichloromethane, and 1.5 mL of trifluoroacetic acid was added. The mixture was stirred at room temperature for 4 h. The reaction liquid was concentrated under reduced pressure, to afford crude 56c trifluoroacetate (85 mg).

### Step 4: 3-chloro-5-(2-(4-((2-(4-(1-((1-(2-(2,6-dioxopiperidin-3-yl)-1,3-dioxoisoindolin-5-yl)piperidin-4-yl)methyl)azetidin-3-yl)piperazin-1-yl)pyrimidin-4-yl)methoxy)phenyl) propan-2-yl)benzonitrile (Compound 56) trifluoroacetate

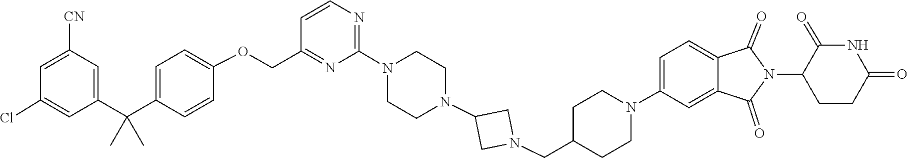 [SMILES: CC(C)(c1ccc(OCc2ccnc(N3CCN(C4CN(CC5CCN(c6ccc7c(c6)C(=O)N(C6CCC(=O)NC6=O)C7=O)CC5)C4)CC3)n2)cc1)c1cc(Cl)cc(C#N)c1]

The above crude 56c trifluoroacetate (85 mg) was dissolved in 6 mL of DMSO, and solid sodium bicarbonate (55 mg, 0.65 mmol), 0.1 mL of DIPEA and 2-(2,6-dioxopiperidin-3-yl)-5-fluoroisoindoline-1,3-dione (see WO 2017197056 for the synthetic method) (43 mg, 0.16 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 56 trifluoroacetate (41 mg).

¹H NMR (400 MHz, CDCl₃) δ 8.38 (d, 1H), 8.27 (br.s, 1H), 7.69 (d, 1H), 7.44 (d, 2H), 7.36-7.32 (m, 1H), 7.26-7.23 (m, 1H), 7.14-7.00 (m, 3H), 6.94-6.83 (m, 3H), 5.01-4.88 (m, 3H), 4.70-4.50 (m, 4H), 4.40-4.05 (m, 5H), 4.00-3.85 (m, 2H), 3.25-3.15 (m, 2H), 3.10-2.65 (m, 9H), 2.20-2.07 (m, 1H), 2.00-1.79 (m, 3H), 1.65 (s, 6H), 1.50-1.30 (m, 2H).

LCMS m/z=856.3 [M+1]⁺.

### Example 57

### 3-chloro-5-(2-(4-((2-(4-(1-((1-(2-(2,6-dioxopiperidin-3-yl)-1,3-dioxoisoindolin-5-yl)azetidin-3-yl)methyl)azetidin-3-yl)piperazin-1-yl)pyrimidin-4-yl)methoxy)phenyl) propan-2-yl)benzonitrile (Compound 57) trifluoroacetate

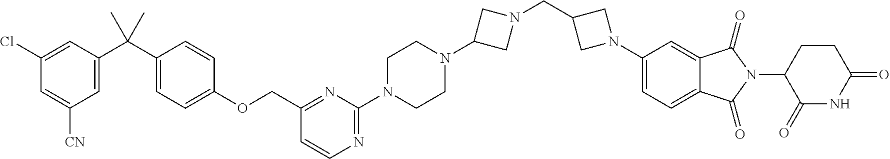 [SMILES: CC(C)(c1ccc(OCc2ccnc(N3CCN(C4CN(CC5CN(c6ccc7c(c6)C(=O)N(C6CCC(=O)NC6=O)C7=O)C5)C4)CC3)n2)cc1)c1cc(Cl)cc(C#N)c1]

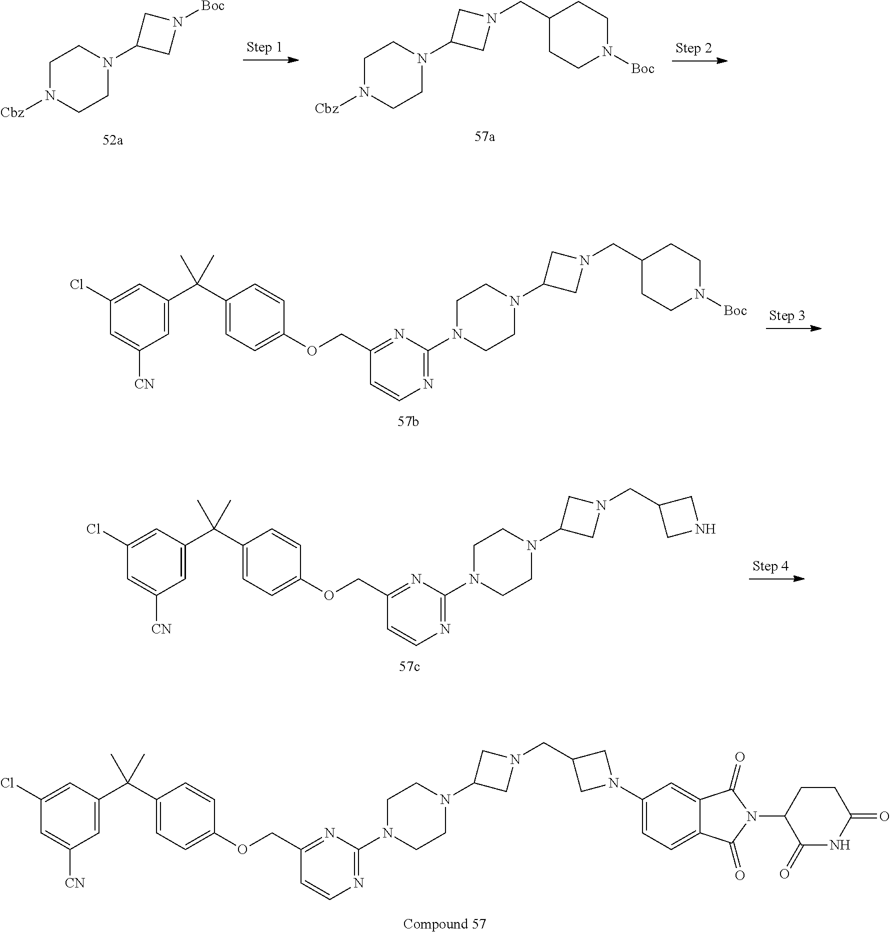 [SMILES: CC(C)(C)OC(=O)N1CC(N2CCN(C(=O)OCc3ccccc3)CC2)C1 ; CC(C)(C)OC(=O)N1CCC(CN2CC(N3CCN(C(=O)OCc4ccccc4)CC3)C2)CC1 ; CC(C)(C)OC(=O)N1CCC(CN2CC(N3CCN(c4nccc(COc5ccc(C(C)(C)c6cc(Cl)cc(C#N)c6)cc5)n4)CC3)C2)CC1 ; CC(C)(c1ccc(OCc2ccnc(N3CCN(C4CN(CC5CNC5)C4)CC3)n2)cc1)c1cc(Cl)cc(C#N)c1 ; CC(C)(c1ccc(OCc2ccnc(N3CCN(C4CN(CC5CN(c6ccc7c(c6)C(=O)N(C6CCC(=O)NC6=O)C7=O)C5)C4)CC3)n2)cc1)c1cc(Cl)cc(C#N)c1]

### Step 1: benzyl 4-(1-((1-(tert-butoxycarbonyl)azetidin-3-yl)methyl)azetidin-3-yl)piperazine-1-carboxylate (57a)

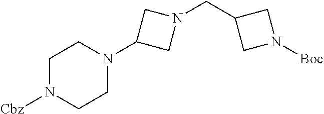 [SMILES: CC(C)(C)OC(=O)N1CC(CN2CC(N3CCN(C(=O)OCc4ccccc4)CC3)C2)C1]

Benzyl 4-(1-(tert-butoxycarbonyl)azetidine-3-yl)piperazine-1-carboxylate (52a) (see WO 2019195609 for the synthetic method) (301 mg, 0.80 mmol) was dissolved in 4 mL of dichloromethane, and 2 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (200 mg, 2.38 mmol) was added, and the mixture was stirred at room temperature for 30 min. Tert-butyl 3-formylazetidine-1-carboxylate (300 mg, 1.62 mmol) was then added, and the mixture was stirred at room temperature for 1 h. Sodium triacetoxyborohydride (680 mg, 3.2 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 57a (325 mg, yield: 91%).

LCMS m/z=445.3 [M+1]⁺

### Step 2: tert-butyl 3-((3-(4-(4-((4-(2-(3-chloro-5-cyanophenyl)propan-2-yl)phenoxy)methyl)pyrimidin-2-yl)piperazin-1-yl)azetidin-1-yl)methyl)azetidine-1-carboxylate (57b)

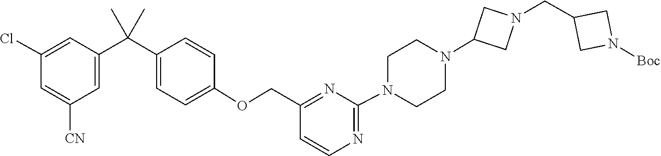 [SMILES: CC(C)(C)OC(=O)N1CC(CN2CC(N3CCN(c4nccc(COc5ccc(C(C)(C)c6cc(Cl)cc(C#N)c6)cc5)n4)CC3)C2)C1]

57a (0.170 g, 0.38 mmol) was dissolved in 5 mL of methanol, and 17 mg of 10% palladium on carbon was added. The mixture was reacted at room temperature under hydrogen atmosphere for 4 h. The reaction liquid was filtered, the filtrate was concentrated under reduced pressure, and the residue was dissolved with 10 mL of DMF. Solid potassium bicarbonate (76 mg, 0.76 mmol) and 3-chloro-5-(2-(4-((2-chloropyrimidin-4-yl)methoxy)phenyl)propan-2-yl)benzonitrile (9d) (150 mg, 0.38 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 57b (128 mg, yield from compound 9d: 50%).

LCMS m/z=672.3 [M+1]⁺

### Step 3: 3-(2-(4-((2-(4-(1-(azetidin-3-ylmethyl)azetidin-3-yl)piperazin-1-yl)pyrimidin-4-yl)methoxy)phenyl)propan-2-yl)-5-chlorobenzonitrile (57c) trifluoroacetate

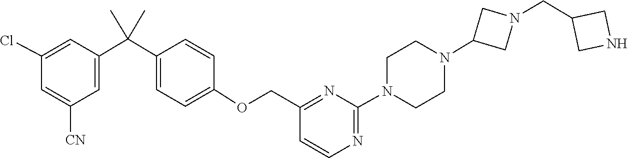 [SMILES: CC(C)(c1ccc(OCc2ccnc(N3CCN(C4CN(CC5CNC5)C4)CC3)n2)cc1)c1cc(Cl)cc(C#N)c1]

57b (128 mg, 0.19 mmol) was dissolved in 3 mL of dichloromethane, and 1.5 mL of trifluoroacetic acid was added. The mixture was stirred at room temperature for 4 h. The reaction liquid was concentrated under reduced pressure, to afford crude 57c trifluoroacetate (130 mg).

### Step 4: 3-chloro-5-(2-(4-((2-(4-(1-((1-(2-(2,6-dioxopiperidin-3-yl)-1,3-dioxoisoindolin-5-yl)azetidin-3-yl)methyl)azetidin-3-yl)piperazin-1-yl)pyrimidin-4-yl)methoxy)phenyl) propan-2-yl)benzonitrile (Compound 57) trifluoroacetate

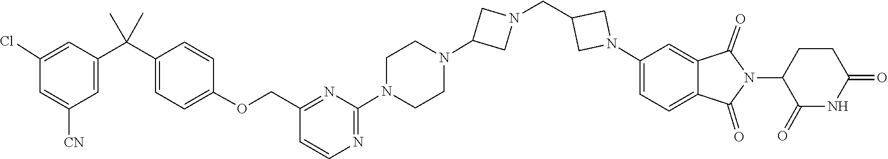 [SMILES: CC(C)(c1ccc(OCc2ccnc(N3CCN(C4CN(CC5CN(c6ccc7c(c6)C(=O)N(C6CCC(=O)NC6=O)C7=O)C5)C4)CC3)n2)cc1)c1cc(Cl)cc(C#N)c1]

The above crude 57c trifluoroacetate (130 mg) was dissolved in 10 mL of DMSO, and solid sodium bicarbonate (80 mg, 0.95 mmol), 0.20 mL of DIPEA and 2-(2,6-dioxopiperidin-3-yl)-5-fluoroisoindoline-1,3-dione (see WO 2017197056 for the synthetic method) (63 mg, 0.23 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 57 trifluoroacetate (53 mg).

¹H NMR (400 MHz, CDCl₃) δ 8.64 (br.s, 1H), 8.38 (d, 1H), 7.64 (d, 1H), 7.48-7.40 (m, 2H), 7.37-7.31 (m, 1H), 7.15-7.05 (m, 2H), 6.97-6.80 (m, 3H), 6.77-6.67 (m, 1H), 6.55-6.44 (m, 1H), 5.03-4.88 (m, 3H), 4.65-4.50 (m, 4H), 4.41-4.05 (m, 7H), 3.84-3.73 (m, 2H), 3.65-3.53 (m, 2H), 3.15-2.94 (m, 5H), 2.94-2.65 (m, 3H), 2.20-2.02 (m, 1H), 1.65 (s, 6H).

LCMS m/z=828.5 [M+1]⁺.

### Example 58

### 3-chloro-5-(2-(4-((2-(4-(1-((1-(2-(2,6-dioxopiperidin-3-yl)-1,3-dioxoisoindolin-5-yl)pyrrolidin-3-yl)methyl)azetidin-3-yl)piperazin-1-yl)pyrimidin-4-yl)methoxy)phenyl) propan-2-yl)benzonitrile (Compound 58) trifluoroacetate

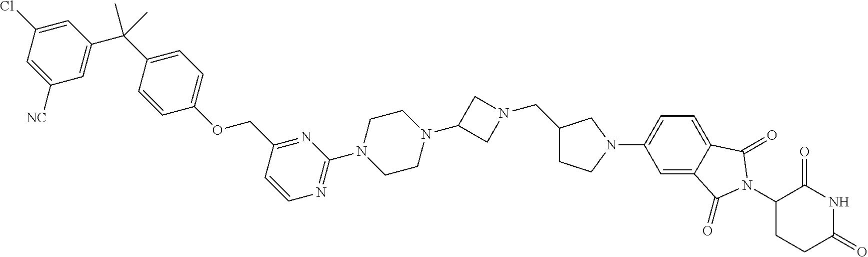 [SMILES: CC(C)(c1ccc(OCc2ccnc(N3CCN(C4CN(CC5CCN(c6ccc7c(c6)C(=O)N(C6CCC(=O)NC6=O)C7=O)C5)C4)CC3)n2)cc1)c1cc(Cl)cc(C#N)c1]

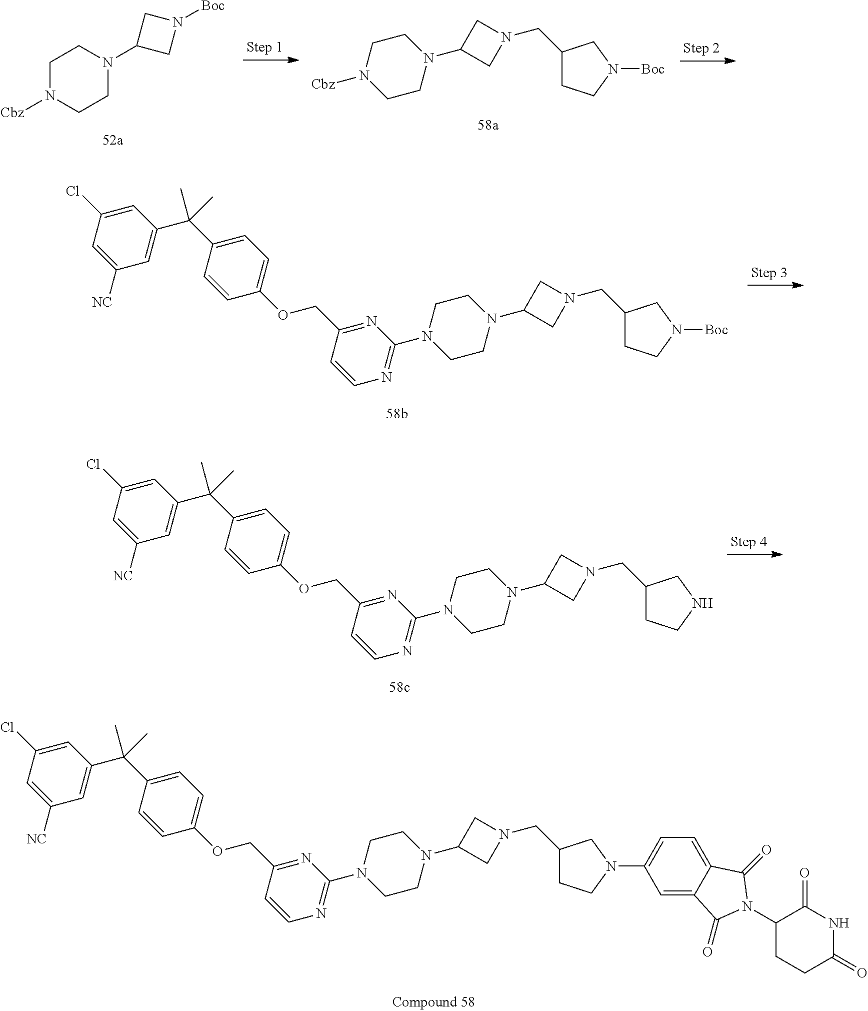 [SMILES: CC(C)(C)OC(=O)N1CC(N2CCN(C(=O)OCc3ccccc3)CC2)C1 ; CC(C)(C)OC(=O)N1CCC(CN2CC(N3CCN(C(=O)OCc4ccccc4)CC3)C2)C1 ; CC(C)(C)OC(=O)N1CCC(CN2CC(N3CCN(c4nccc(COc5ccc(C(C)(C)c6cc(Cl)cc(C#N)c6)cc5)n4)CC3)C2)C1 ; CC(C)(c1ccc(OCc2ccnc(N3CCN(C4CN(CC5CCNC5)C4)CC3)n2)cc1)c1cc(Cl)cc(C#N)c1 ; CC(C)(c1ccc(OCc2ccnc(N3CCN(C4CN(CC5CCN(c6ccc7c(c6)C(=O)N(C6CCC(=O)NC6=O)C7=O)C5)C4)CC3)n2)cc1)c1cc(Cl)cc(C#N)c1]

### Step 1: benzyl 4-(1-((1-(tert-butoxycarbonyl)pyrrolidin-3-yl)methyl)azetidin-3-yl)piperazine-1-carboxylate (58a)

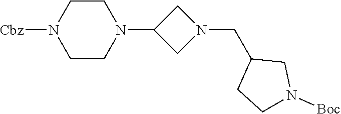 [SMILES: CC(C)(C)OC(=O)N1CCC(CN2CC(N3CCN(C(=O)OCc4ccccc4)CC3)C2)C1]

Benzyl 4-(1-(tert-butoxycarbonyl)azetidine-3-yl)piperazine-1-carboxylate (52a) (see WO 2019195609 for the synthetic method) (120 mg, 0.32 mmol) was dissolved in 2 mL of dichloromethane, and 1 mL of trifluoroacetic acid was added. The mixture was stirred at room temperature for 4 h. The reaction liquid was concentrated under reduced pressure, and the residue was dissolved with 5 mL of tetrahydrofuran. Solid sodium bicarbonate (81 mg, 0.96 mmol) was added, and the mixture was stirred at room temperature for 30 min. Tert-butyl 3-formylpyrrolidine-1-carboxylate (64 mg, 0.32 mmol) was then added, and the mixture was stirred at room temperature for 1 h. Sodium triacetoxyborohydride (270 mg, 1.27 mmol) was then added, and the resulting mixture was reacted at room temperature for 16 h. To the reaction liquid was slowly added 20 mL of saturated sodium bicarbonate solution, and the mixture was extracted with 40 mL of dichloromethane. The organic phase was washed with 60 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 58a (130 mg, yield: 89%).

LCMS m/z=459.2 [M+1]⁺

### Step 2: tert-butyl 3-((3-(4-(4-((4-(2-(3-chloro-5-cyanophenyl)propan-2-yl)phenoxy)methyl)pyrimidin-2-yl)piperazin-1-yl)azetidin-1-yl)methyl)pyrrolidine-1-carboxylate (58b)

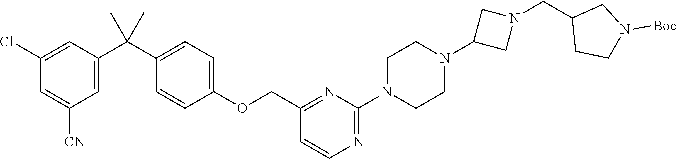 [SMILES: CC(C)(C)OC(=O)N1CCC(CN2CC(N3CCN(c4nccc(COc5ccc(C(C)(C)c6cc(Cl)cc(C#N)c6)cc5)n4)CC3)C2)C1]

58a (0.130 g, 0.28 mmol) was dissolved in 5 mL of methanol, and 13 mg of 10% palladium on carbon was added. The mixture was reacted at room temperature under hydrogen atmosphere for 4 h. The reaction liquid was filtered, the filtrate was concentrated under reduced pressure, and the residue was dissolved with 5 mL of DMF. Solid potassium bicarbonate (56 mg, 0.56 mmol) and 3-chloro-5-(2-(4-((2-chloropyrimidin-4-yl)methoxy)phenyl)propan-2-yl)benzonitrile (9d) (110 mg, 0.28 mmol) were added, and the mixture was warmed to 60° C. and reacted for 3 h. The reaction liquid was cooled to room temperature, and 80 mL of water was slowly added. The mixture was extracted with 90 mL of ethyl acetate, and the organic phase was washed with 40 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 58b (78 mg, yield from compound 9d: 41%).

LCMS m/z=686.3 [M+1]⁺

### Step 3: 3-chloro-5-(2-(4-((2-(4-(1-(pyrrolidin-3-ylmethyl)azetidin-3-yl)piperazin-1-yl)pyrimidin-4-yl)methoxy)phenyl)propan-2-yl)benzonitrile (58c) trifluoroacetate

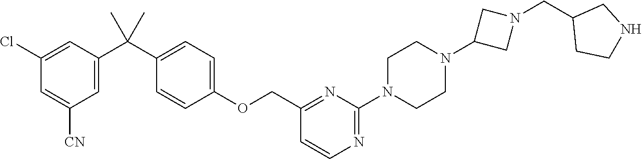 [SMILES: CC(C)(c1ccc(OCc2ccnc(N3CCN(C4CN(CC5CCNC5)C4)CC3)n2)cc1)c1cc(Cl)cc(C#N)c1]

58b (75 mg, 0.11 mmol) was dissolved in 3 mL of dichloromethane, and 1.5 mL of trifluoroacetic acid was added. The mixture was stirred at room temperature for 4 h. The reaction liquid was concentrated under reduced pressure, to afford crude 58c trifluoroacetate (88 mg).

### Step 4: 3-chloro-5-(2-(4-((2-(4-(1-((1-(2-(2,6-dioxopiperidin-3-yl)-1,3-dioxoisoindolin-5-yl)pyrrolidin-3-yl)methyl)azetidin-3-yl)piperazin-1-yl)pyrimidin-4-yl)methoxy)phenyl) propan-2-yl)benzonitrile (Compound 58) trifluoroacetate

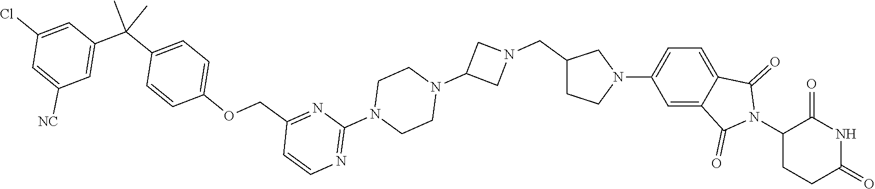 [SMILES: CC(C)(c1ccc(OCc2ccnc(N3CCN(C4CN(CC5CCN(c6ccc7c(c6)C(=O)N(C6CCC(=O)NC6=O)C7=O)C5)C4)CC3)n2)cc1)c1cc(Cl)cc(C#N)c1]

The above crude 58c trifluoroacetate (88 mg) was dissolved in 5 mL of DMSO, and solid sodium bicarbonate (46 mg, 0.55 mmol), 0.15 mL of DIPEA and 2-(2,6-dioxopiperidin-3-yl)-5-fluoroisoindoline-1,3-dione (see WO 2017197056 for the synthetic method) (36 mg, 0.13 mmol) were added. The reaction was stirred at 80° C. for 5 h. The reaction liquid was cooled to room temperature, and 50 mL of water was added. The mixture was extracted with 100 mL of ethyl acetate, and the organic phase was washed with 100 mL of water, dried over anhydrous sodium sulfate, and concentrated under reduced pressure, to afford a crude product. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 58 trifluoroacetate (24 mg).

¹H NMR (400 MHz, CDCl₃) δ 8.35 (d, 1H), 7.61 (d, 1H), 7.44 (d, 2H), 7.38-7.32 (m, 1H), 7.28-7.23 (m, 1H), 7.14-7.06 (m, 2H), 6.94-6.78 (m, 4H), 6.66-6.52 (m, 1H), 5.01-4.88 (m, 3H), 4.65-4.46 (m, 2H), 4.44-4.23 (m, 2H), 4.17-3.90 (m, 5H), 3.65-3.20 (m, 5H), 3.18-3.05 (m, 1H), 2.97-2.68 (m, 7H), 2.68-2.50 (m, 1H), 2.36-2.22 (m, 1H), 2.20-2.06 (m, 1H), 1.90-1.72 (m, 1H), 1.65 (s, 6H).

LCMS m/z=842.5 [M+1]⁺.

### Example 59

### 3-chloro-5-(2-(4-((2-(4-(7-(2-(2,6-dioxopiperidin-3-yl)-1,3-dioxoisoindolin-5-yl)-7,7′-diaza[2,7′-bispiro[3.5]nonan]-2′-yl)piperazin-1-yl)pyrimidin-4-yl)methoxy)phenyl)propan-2-yl)benzonitrile (Compound 59) trifluoroacetate

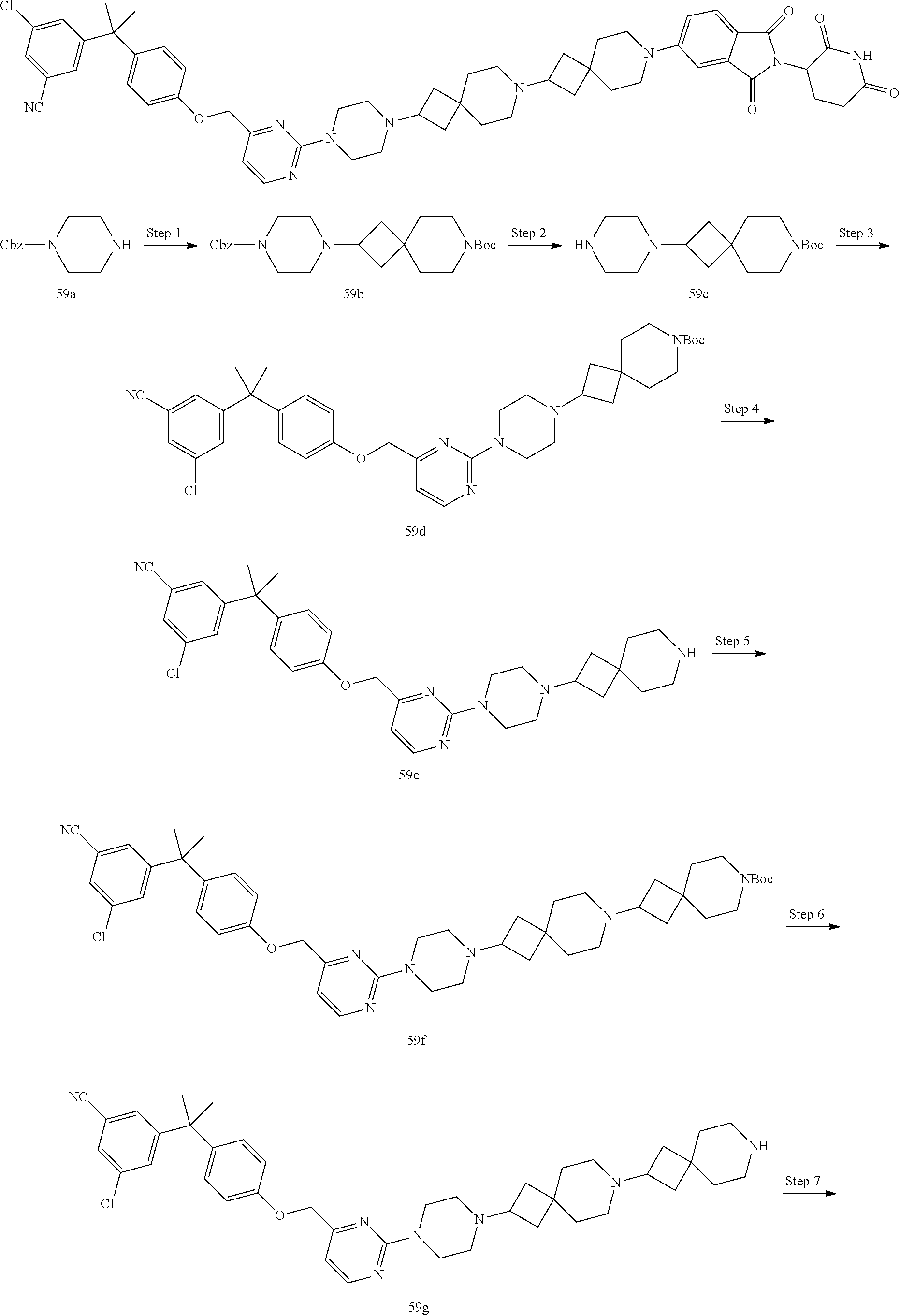

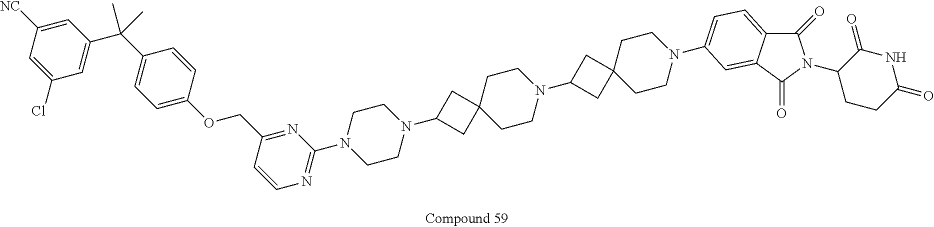 [SMILES: CC(C)(c1ccc(OCc2ccnc(N3CCN(C4CC5(CCN(C6CC7(CCN(c8ccc9c(c8)C(=O)N(C8CCC(=O)NC8=O)C9=O)CC7)C6)CC5)C4)CC3)n2)cc1)c1cc(Cl)cc(C#N)c1]

### Step 1: tert-buty 12-(4-((benzyloxy)carbonyl)piperazin-1-yl)-7-azaspiro[3.5]nonane-7-carboxylate (59b)

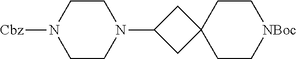 [SMILES: CC(C)(C)OC(=O)N1CCC2(CC1)CC(N1CCN(C(=O)OCc3ccccc3)CC1)C2]

Benzyl piperazine-1-carboxylate (59a) (2.2 g, 10 mmol) and tert-butyl 2-oxo-7-azaspiro[3.5]nonane-7-carboxylate (2.4 g, 10 mmol) were added to 100 mL of dichloromethane, and the mixture was reacted at room temperature for 2 h. Sodium triacetoxyborohydride (2.1 g, 10 mmol) was then added, and the mixture was reacted at room temperature for 12 h. To the reaction liquid were added 100 mL of dichloromethane and 100 mL of 1 mol/L aqueous sodium hydroxide solution. The organic phase was separated, dried over anhydrous sodium sulfate, and concentrated under reduced pressure. The crude product was separated and purified by silica gel column chromatography (ethyl acetate/petroleum ether (v/v)=1: 10-1:1), to afford 59b (4 g, yield: 90%).

LCMS m/z=444.3 [M+1]⁺

### Step 2: tert-butyl 2-(piperazin-1-yl)-7-azaspiro[3.5]nonane-7-carboxylate (59c)

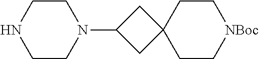 [SMILES: CC(C)(C)OC(=O)N1CCC2(CC1)CC(N1CCNCC1)C2]

59b (4 g, 9 mmol) was dissolved in methanol (100 mL), and 0.5 g of 10% palladium on carbon was added. The mixture was reacted at room temperature under hydrogen atmosphere for 16 h. The reaction system was filtered, and the filtrate was concentrated under reduced pressure, to afford crude 59c (2.5 g).

LCMS m/z=310.2 [M+1]⁺

### Step 3: tert-butyl 2-(4-(4-((4-(2-(3-chloro-5-cyanophenyl)propan-2-yl)phenoxy)methyl)pyrimidin-2-yl)piperazin-1-yl)-7-azaspiro[3.5]nonane-7-carboxylate (59d)

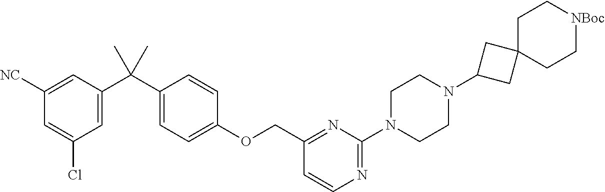 [SMILES: CC(C)(C)OC(=O)N1CCC2(CC1)CC(N1CCN(c3nccc(COc4ccc(C(C)(C)c5cc(Cl)cc(C#N)c5)cc4)n3)CC1)C2]

3-chloro-5-(2-(4-((2-chloropyrimidin-4-yl)methoxy)phenyl)propan-2-yl)benzonitrile (9d) (740 mg, 1.86 mmol) was dissolved in 15 mL of DMF, and the above crude 59c (760 mg) and solid potassium bicarbonate (690 mg, 6.9 mmol) were successively added. The mixture was warmed to 75° C. and reacted for 4 h. The reaction liquid was cooled to room temperature, and 60 mL of water was added. The mixture was extracted twice with 60 mL of ethyl acetate, and the organic phase was washed with 60 mL of saturated sodium chloride solution, dried over anhydrous sodium sulfate, and concentrated under reduced pressure.

The crude product was separated and purified by silica gel column chromatography (petroleum ether/ethyl acetate (v/v)=1:1), to afford 59d (1000 mg, yield from compound 9d: 80%).

LCMS m/z=671.3 [M+1]⁺

### Step 4: 3-(2-(4-((2-(4-(7-azaspiro[3.5]nonan-2-yl)piperazin-1-yl)pyrimidin-4-yl)methoxy)phenyl)propan-2-yl)-5-chlorobenzonitrile (59e)

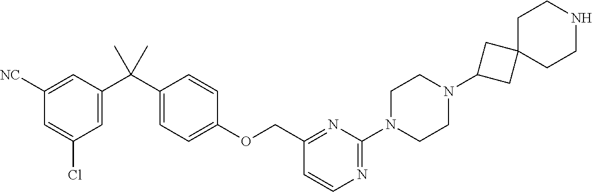 [SMILES: CC(C)(c1ccc(OCc2ccnc(N3CCN(C4CC5(CCNCC5)C4)CC3)n2)cc1)c1cc(Cl)cc(C#N)c1]

59d (1000 mg, 1.49 mmol) was dissolved in 30 mL of dichloromethane, and 10 mL of trifluoroacetic acid was added. The mixture was reacted at room temperature for 2 h. The reaction system was concentrated under reduced pressure, and 100 mL of dichloromethane was added. The mixture was adjusted to pH 9.0 with a saturated sodium bicarbonate solution, and the aqueous phase was extracted with 50 mL of dichloromethane. The organic phases were combined, dried over anhydrous sodium sulfate, and concentrated under reduced pressure, to afford crude 59e (800 mg).

LCMS m/z=571.3 [M+1]⁺

### Step 5: tert-butyl 2′-(4-(4-((4-(2-(3-chloro-5-cyanophenyl)propan-2-yl)phenoxy)methyl)pyrimidin-2-yl)piperazin-1-yl)-7,7′-diaza[2,7′-bispiro[3.5]nonane]-7-carboxylate (59f)

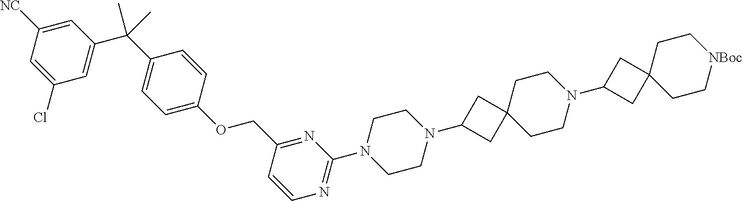 [SMILES: CC(C)(C)OC(=O)N1CCC2(CC1)CC(N1CCC3(CC1)CC(N1CCN(c4nccc(COc5ccc(C(C)(C)c6cc(Cl)cc(C#N)c6)cc5)n4)CC1)C3)C2]

The above crude 59e (97 mg) was added to 10 mL of 1,2-dichloroethane, and tert-butyl 2-oxo-7-azaspiro[3.5]nonane-7-carboxylate (72 mg, 0.3 mmol) was added. The mixture was reacted at room temperature for 1 h, and then sodium triacetoxyborohydride (63 mg, 0.3 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 59f (90 mg, two-step yield from compound 59d: 65%).

LCMS m/z=794.4 [M+1]⁺

### Step 6: 3-(2-(4-((2-(4-(7,7′-diaza[2,7′-bispiro[3.5]nonan]-2′-yl)piperazin-1-yl)pyrimidin-4-yl)methoxy)phenyl)propan-2-yl)-5-chlorobenzonitrile (59g)

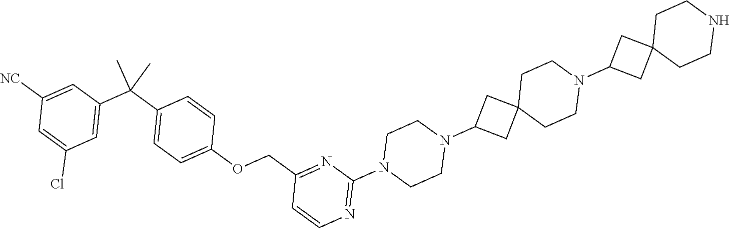 [SMILES: CC(C)(c1ccc(OCc2ccnc(N3CCN(C4CC5(CCN(C6CC7(CCNCC7)C6)CC5)C4)CC3)n2)cc1)c1cc(Cl)cc(C#N)c1]

59f (90 mg, 0.11 mmol) was dissolved in 6 mL of dichloromethane, and 2 mL of trifluoroacetic acid was added. The mixture was reacted at room temperature for 2 h. The reaction liquid was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 59g (80 mg).

### Step 7: 3-chloro-5-(2-(4-((2-(4-(7-(2-(2,6-dioxopiperidin-3-yl)-1,3-dioxoisoindolin-5-yl)-7,7′-diaza[2,7′-bispiro[3.5]nonan]-2′-yl)piperazin-1-yl)pyrimidin-4-yl)methoxy)phenyl)propan-2-yl)benzonitrile (Compound 59) trifluoroacetate

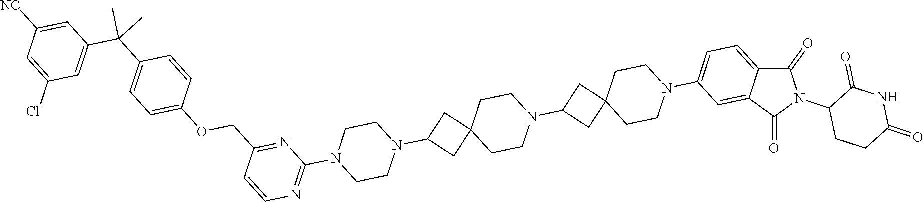 [SMILES: CC(C)(c1ccc(OCc2ccnc(N3CCN(C4CC5(CCN(C6CC7(CCN(c8ccc9c(c8)C(=O)N(C8CCC(=O)NC8=O)C9=O)CC7)C6)CC5)C4)CC3)n2)cc1)c1cc(Cl)cc(C#N)c1]

The above crude 59g (80 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 59 trifluoroacetate (32 mg).

¹H NMR (400 MHz, DMSO-d₆) δ 10.06 (s, 1H), 8.47 (d, 1H), 7.86-7.82 (m, 1H), 7.69-7.61 (m, 2H), 7.54-7.49 (m, 1H), 7.37-7.22 (m, 2H), 7.21-7.13 (m, 2H), 6.98-6.91 (m, 2H), 6.86 (d, 1H), 5.11-4.98 (m, 3H), 4.90-4.55 (m, 2H), 3.85-3.66 (m, 2H), 3.50-3.36 (m, 4H), 3.36-3.18 (m, 2H), 2.98-2.72 (m, 3H), 2.71-2.50 (m, 7H), 2.41-2.29 (m, 1H), 2.26-2.11 (m, 4H), 2.07-1.95 (m, 4H), 1.95-1.86 (m, 1H), 1.85-1.69 (m, 3H), 1.69-1.57 (m, 11H).

LCMS m/z=475.8 [M/2+1]⁺

### Example 60

### 3-chloro-5-(2-(4-((2-(4-(7-((1-(2-(2,6-dioxopiperidin-3-yl)-1,3-dioxoisoindolin-5-yl)piperidin-4-yl)methyl)-7-azaspiro[3.5]nonan-2-yl)piperazin-1-yl)pyrimidin-4-yl)methoxy)phenyl)propan-2-yl)benzonitrile (Compound 60) trifluoroacetate

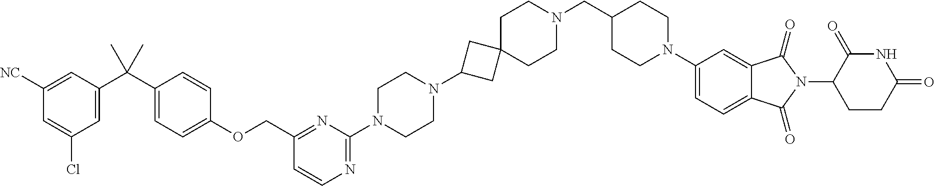 [SMILES: CC(C)(c1ccc(OCc2ccnc(N3CCN(C4CC5(CCN(CC6CCN(c7ccc8c(c7)C(=O)N(C7CCC(=O)NC7=O)C8=O)CC6)CC5)C4)CC3)n2)cc1)c1cc(Cl)cc(C#N)c1]

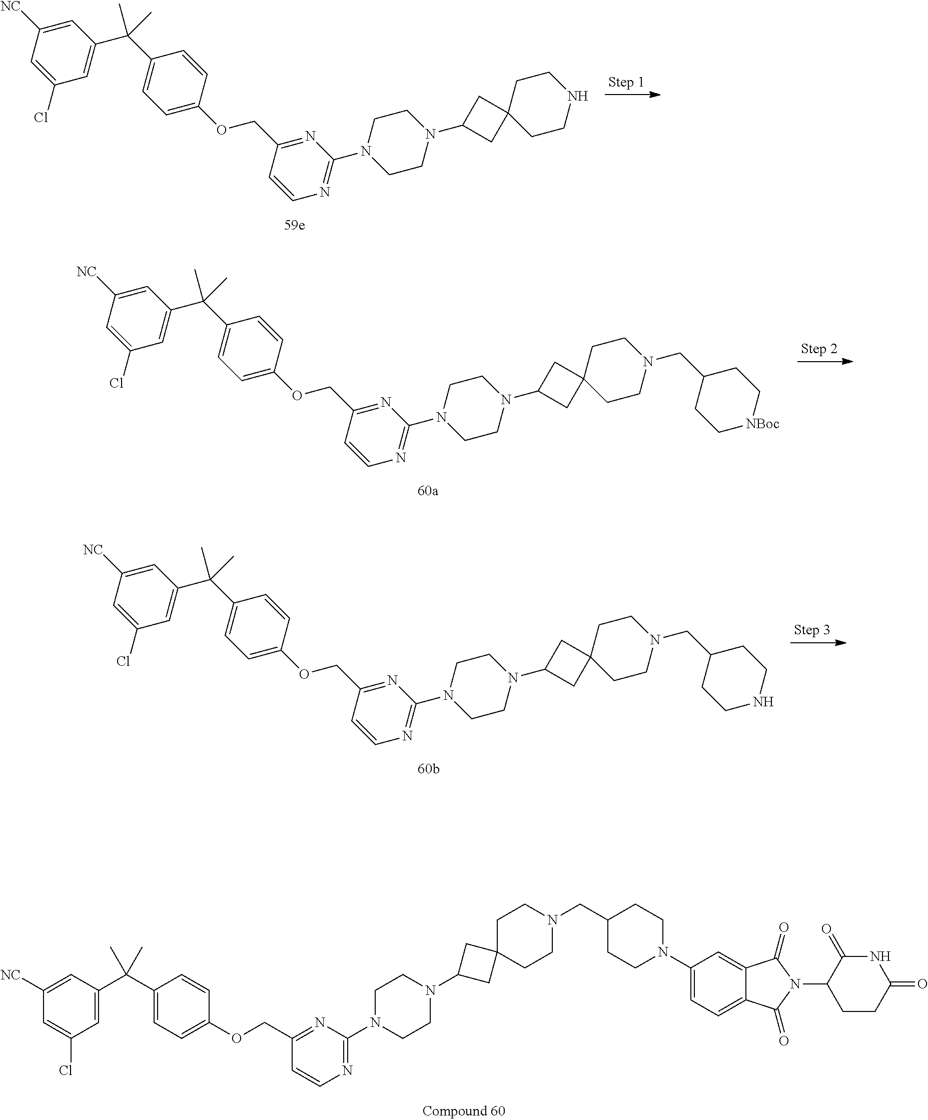 [SMILES: CC(C)(c1ccc(OCc2ccnc(N3CCN(C4CC5(CCNCC5)C4)CC3)n2)cc1)c1cc(Cl)cc(C#N)c1 ; CC(C)(C)OC(=O)N1CCC(CN2CCC3(CC2)CC(N2CCN(c4nccc(COc5ccc(C(C)(C)c6cc(Cl)cc(C#N)c6)cc5)n4)CC2)C3)CC1 ; CC(C)(c1ccc(OCc2ccnc(N3CCN(C4CC5(CCN(CC6CCNCC6)CC5)C4)CC3)n2)cc1)c1cc(Cl)cc(C#N)c1 ; CC(C)(c1ccc(OCc2ccnc(N3CCN(C4CC5(CCN(CC6CCN(c7ccc8c(c7)C(=O)N(C7CCC(=O)NC7=O)C8=O)CC6)CC5)C4)CC3)n2)cc1)c1cc(Cl)cc(C#N)c1]

### Step 1: tert-butyl 4-((2-(4-(4-((4-(2-(3-chloro-5-cyanophenyl)propan-2-yl)phenoxy)methyl)pyrimidin-2-yl)piperazin-1-yl)-7-azaspiro[3.5]nonan-7-yl)methyl)piperidine-1-carboxylate (60a)

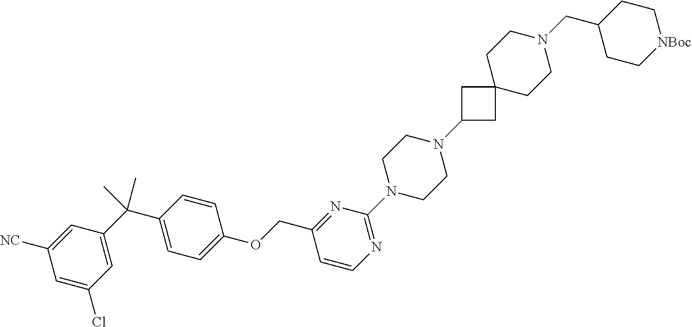 [SMILES: CC(C)(C)OC(=O)N1CCC(CN2CCC3(CC2)CC(N2CCN(c4nccc(COc5ccc(C(C)(C)c6cc(Cl)cc(C#N)c6)cc5)n4)CC2)C3)CC1]

The above crude 59e (90 mg) was added to 10 mL of 1,2-dichloroethane, and tert-butyl 4-formylpiperidine-1-carboxylate (64 mg, 0.3 mmol) was added. The mixture was reacted at room temperature for 1 h, and then sodium triacetoxyborohydride (63 mg, 0.3 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 60a (90 mg, two-step yield from compound 59d: 70%).

LCMS m/z=768.5 [M+1]⁺

### Step 2: 3-chloro-5-(2-(4-((2-(4-(7-(piperidin-4-ylmethyl)-7-azaspiro[3.5]nonan-2-yl)piperazin-1-yl)pyrimidin-4-yl)methoxy)phenyl)propan-2-yl)benzonitrile (60b)

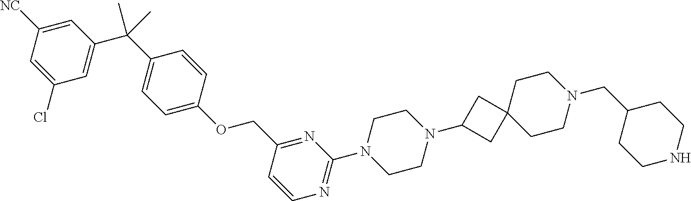 [SMILES: CC(C)(c1ccc(OCc2ccnc(N3CCN(C4CC5(CCN(CC6CCNCC6)CC5)C4)CC3)n2)cc1)c1cc(Cl)cc(C#N)c1]

60a (90 mg, 0.12 mmol) was dissolved in 6 mL of dichloromethane, and 2 mL of trifluoroacetic acid was added. The mixture was reacted at room temperature for 2 h. The reaction system was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 60b (72 mg).

### Step 3: 3-chloro-5-(2-(4-((2-(4-(7-((1-(2-(2,6-dioxopiperidin-3-yl)-1,3-dioxoisoindolin-5-yl)piperidin-4-yl)methyl)-7-azaspiro[3.5]nonan-2-yl)piperazin-1-yl)pyrimidin-4-yl)methoxy)phenyl)propan-2-yl)benzonitrile (Compound 60) trifluoroacetate

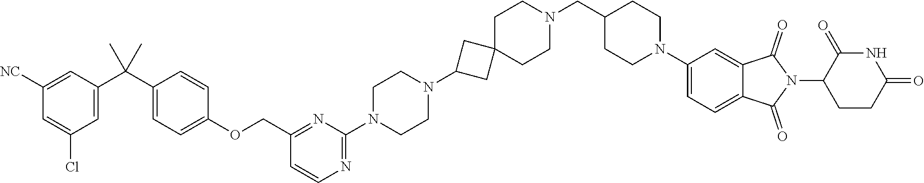 [SMILES: CC(C)(c1ccc(OCc2ccnc(N3CCN(C4CC5(CCN(CC6CCN(c7ccc8c(c7)C(=O)N(C7CCC(=O)NC7=O)C8=O)CC6)CC5)C4)CC3)n2)cc1)c1cc(Cl)cc(C#N)c1]

The above crude 60b (72 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 60 trifluoroacetate (25 mg).

¹H NMR (400 MHz, DMSO-d₆) δ 11.06 (s, 1H), 8.46 (d, 1H), 7.87-7.83 (m, 1H), 7.71-7.62 (m, 2H), 7.55-7.50 (m, 1H), 7.39-7.32 (m, 1H), 7.30-7.22 (m, 1H), 7.21-7.13 (m, 2H), 7.00-6.82 (m, 3H), 5.13-4.98 (m, 3H), 4.85-4.65 (m, 2H), 4.17-4.00 (m, 2H), 3.85-3.70 (m, 2H), 3.55-3.35 (m, 5H), 3.35-3.15 (m, 2H), 3.10-2.75 (m, 9H), 2.70-2.53 (m, 2H), 2.41-2.28 (m, 1H), 2.21-1.96 (m, 4H), 1.92-1.70 (m, 5H), 1.64 (s, 6H), 1.37-1.17 (m, 2H).

LCMS m/z=462.8 [M/2+1]⁺

### Example 61

### 3-chloro-5-(2-(4-((2-(4-(1-((1-(2-(2,6-dioxopiperidin-3-yl)-1,3-dioxoisoindolin-5-yl)azetidin-3-yl)methyl)pyrrolidin-3-yl)piperazin-1-yl)pyrimidin-4-yl)methoxy)phenyl) propan-2-yl)benzonitrile (Compound 61) trifluoroacetate

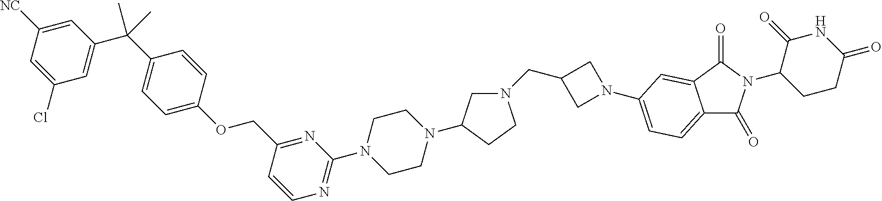 [SMILES: CC(C)(c1ccc(OCc2ccnc(N3CCN(C4CCN(CC5CN(c6ccc7c(c6)C(=O)N(C6CCC(=O)NC6=O)C7=O)C5)C4)CC3)n2)cc1)c1cc(Cl)cc(C#N)c1]

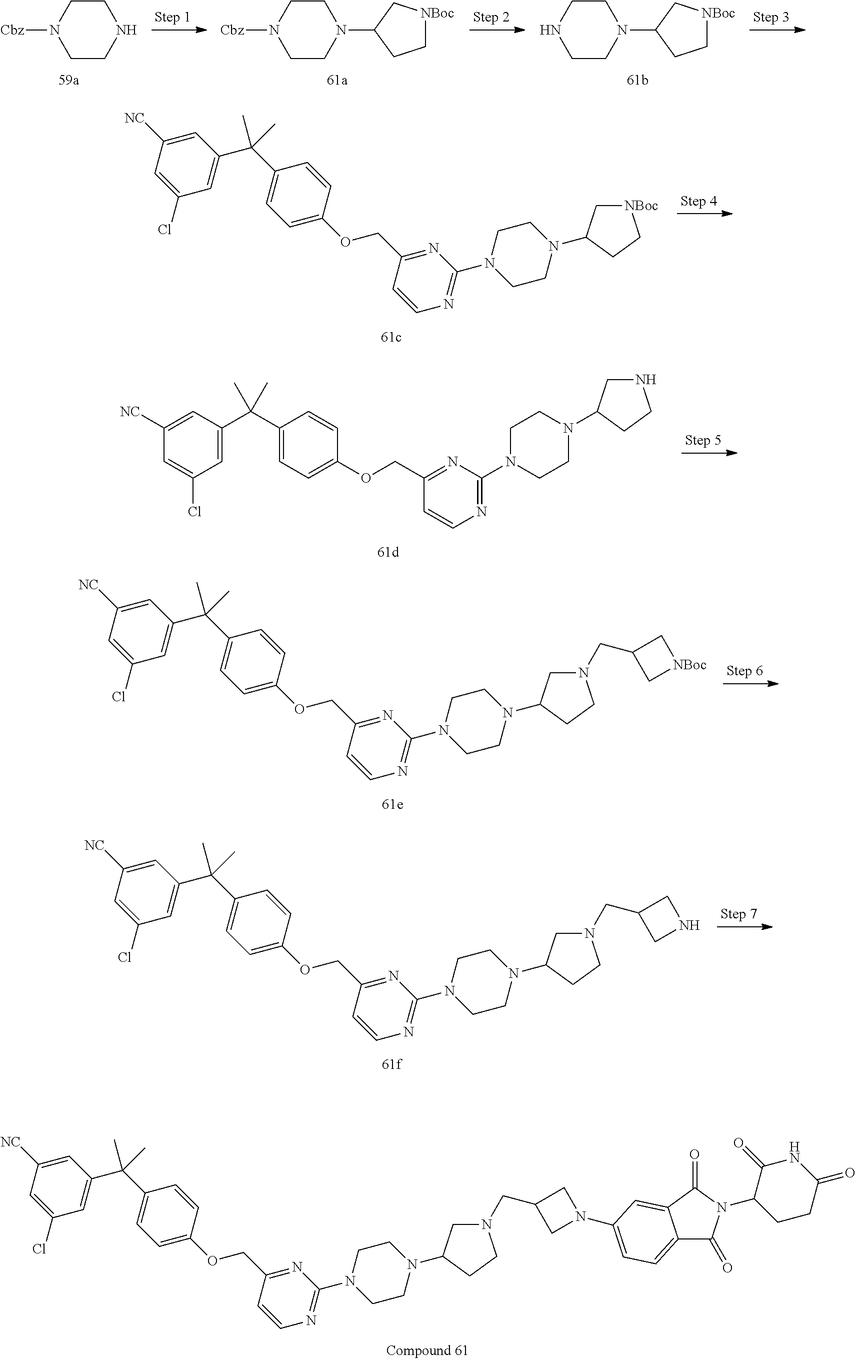

### Step 1: benzyl 4-(1-(tert-butoxycarbonyl)pyrrolidin-3-yl)piperazine-1-carboxylate (61a)

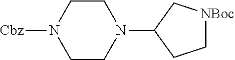 [SMILES: CC(C)(C)OC(=O)N1CCC(N2CCN(C(=O)OCc3ccccc3)CC2)C1]

Benzyl piperazine-1-carboxylate (59a) (6.6 g, 30 mmol) and tert-butyl 3-oxopyrrolidine-1-carboxylate (6.0 g, 32.4 mmol) were added to 100 mL of dichloromethane, and the mixture was reacted at room temperature for 2 h. Sodium triacetoxyborohydride (7.0 g, 33 mmol) was then added, and the mixture was reacted at room temperature for 12 h. To the reaction liquid were added 100 mL of dichloromethane and 100 mL of 1 mol/L aqueous sodium hydroxide solution. The organic phase was separated, dried over anhydrous sodium sulfate, and concentrated under reduced pressure. The crude product was separated and purified by silica gel column chromatography (ethyl acetate/petroleum ether (v/v)=1: 10-1:1), to afford 61a (9.0 g, yield: 77%).

LCMS m/z=390.3 [M+1]⁺

### Step 2: tert-butyl 3-(piperazin-1-yl)pyrrolidine-1-carboxylate (61b)

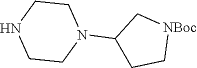 [SMILES: CC(C)(C)OC(=O)N1CCC(N2CCNCC2)C1]

61a (9.0 g, 23.11 mmol) was dissolved in methanol (100 mL), and 0.5 g of 10% palladium on carbon was added. The mixture was reacted at room temperature under hydrogen atmosphere for 16 h. The reaction system was filtered, and the filtrate was concentrated under reduced pressure, to afford crude 61b (5.5 g).

LCMS m/z=256.3 [M+1]⁺

### Step 3: tert-butyl 3-(4-(4-((4-(2-(3-chloro-5-cyanophenyl)propan-2-yl)phenoxy)methyl)pyrimidin-2-yl)piperazin-1-yl)pyrrolidine-1-carboxylate (61c)

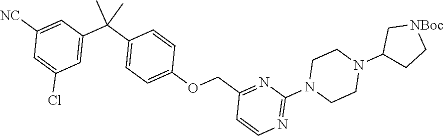 [SMILES: CC(C)(C)OC(=O)N1CCC(N2CCN(c3nccc(COc4ccc(C(C)(C)c5cc(Cl)cc(C#N)c5)cc4)n3)CC2)C1]

3-chloro-5-(2-(4-((2-chloropyrimidin-4-yl)methoxy)phenyl)propan-2-yl)benzonitrile (9d) (1.6 g, 4.02 mmol) was dissolved in 25 mL of DMF, and the above crude 61b (1.3 g) and solid potassium bicarbonate (1.1 g, 11.0 mmol) were successively added. The mixture was warmed to 75° C. and reacted for 4 h. The reaction liquid was cooled to room temperature, and 80 mL of water was added. The mixture was extracted twice with 80 mL of ethyl acetate, and the organic phase was washed with 60 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 61c (1.35 g, yield from compound 9d: 54%).

LCMS m/z=617.3 [M+1]⁺

### Step 4: 3-chloro-5-(2-(4-((2-(4-(pyrrolidin-3-yl)piperazin-1-yl)pyrimidin-4-yl)methoxy)phenyl)propan-2-yl)benzonitrile (61d)

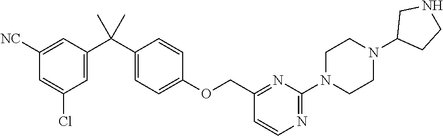 [SMILES: CC(C)(c1ccc(OCc2ccnc(N3CCN(C4CCNC4)CC3)n2)cc1)c1cc(Cl)cc(C#N)c1]

61c (1.35 g, 2.2 mmol) was dissolved in 30 mL of dichloromethane, and 10 mL of trifluoroacetic acid was added. The mixture was reacted at room temperature for 2 h. The reaction system was concentrated under reduced pressure, and 100 mL of dichloromethane was added. The mixture was adjusted to pH 9.0 with a saturated sodium bicarbonate solution, and the aqueous phase was extracted with 50 mL of dichloromethane. The organic phases were combined, dried over anhydrous sodium sulfate, and concentrated under reduced pressure, to afford crude 61d (950 mg).

LCMS m/z=517.3 [M+1]⁺

### Step 5: tert-butyl 3-((3-(4-(4-((4-(2-(3-chloro-5-cyanophenyl)propan-2-yl)phenoxy)methyl)pyrimidin-2-yl)piperazin-1-yl)pyrrolidin-1-yl)methyl)azetidine-1-carboxylate (61e)

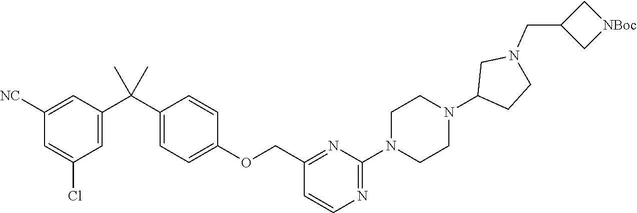 [SMILES: CC(C)(C)OC(=O)N1CC(CN2CCC(N3CCN(c4nccc(COc5ccc(C(C)(C)c6cc(Cl)cc(C#N)c6)cc5)n4)CC3)C2)C1]

The above crude 61d (90 mg) was added to 10 mL of 1,2-dichloroethane, and tert-butyl 3-formylazetidine-1-carboxylate (37 mg, 0.2 mmol) was added. The mixture was reacted at room temperature for 1 h, and then sodium triacetoxyborohydride (42 mg, 0.2 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 61e (100 mg, two-step yield from compound 61c: 70%).

LCMS m/z=686.4 [M+1]⁺

### Step 6: 3-(2-(4-((2-(4-(1-(azetidin-3-ylmethyl)pyrrolidin-3-yl)piperazin-1-yl)pyrimidin-4-yl)methoxy)phenyl)propan-2-yl)-5-chlorobenzonitrile (61f)

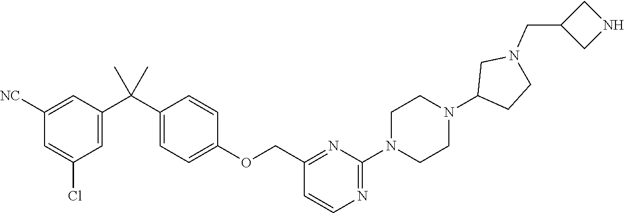 [SMILES: CC(C)(c1ccc(OCc2ccnc(N3CCN(C4CCN(CC5CNC5)C4)CC3)n2)cc1)c1cc(Cl)cc(C#N)c1]

61e (100 mg, 0.15 mmol) was dissolved in 6 mL of dichloromethane, and 2 mL of trifluoroacetic acid was added. The mixture was reacted at room temperature for 2 h. The reaction liquid was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 61f (90 mg).

### Step 7: 3-chloro-5-(2-(4-((2-(4-(1-((1-(2-(2,6-dioxopiperidin-3-yl)-1,3-dioxoisoindolin-5-yl)azetidin-3-yl)methyl)pyrrolidin-3-yl)piperazin-1-yl)pyrimidin-4-yl)methoxy)phenyl) propan-2-yl)benzonitrile (Compound 61) trifluoroacetate

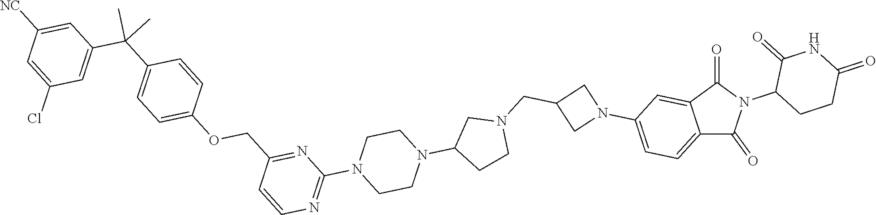 [SMILES: CC(C)(c1ccc(OCc2ccnc(N3CCN(C4CCN(CC5CN(c6ccc7c(c6)C(=O)N(C6CCC(=O)NC6=O)C7=O)C5)C4)CC3)n2)cc1)c1cc(Cl)cc(C#N)c1]

The above crude 61f (90 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 61 trifluoroacetate (22 mg).

¹H NMR (400 MHz, DMSO-d₆) δ 11.05 (s, 1H), 8.42 (d, 1H), 7.88-7.82 (m, 1H), 7.72-7.62 (m, 2H), 7.56-7.50 (m, 1H), 7.20-7.14 (m, 2H), 6.99-6.90 (m, 2H), 6.84-6.76 (m, 2H), 6.68 (dd, 1H), 5.11-4.96 (m, 3H), 4.27-4.15 (m, 2H), 3.94-3.75 (m, 6H), 3.24-3.10 (m, 5H), 3.00-2.76 (m, 5H), 2.70-2.51 (m, 3H), 2.31-1.93 (m, 5H), 1.64 (s, 6H).

LCMS m/z=842.2 [M+1]⁺

### Example 62: 3-chloro-5-(2-(4-((2-(4-(1-((1-(2-(2,6-dioxopiperidin-3-yl)-1,3-dioxoisoindolin-5-yl)pyrrolidin-3-yl)methyl)pyrrolidin-3-yl)piperazin-1-yl)pyrimidin-4-yl)methoxy)phenyl)propan-2-yl)benzonitrile (Compound 62) trifluoroacetate

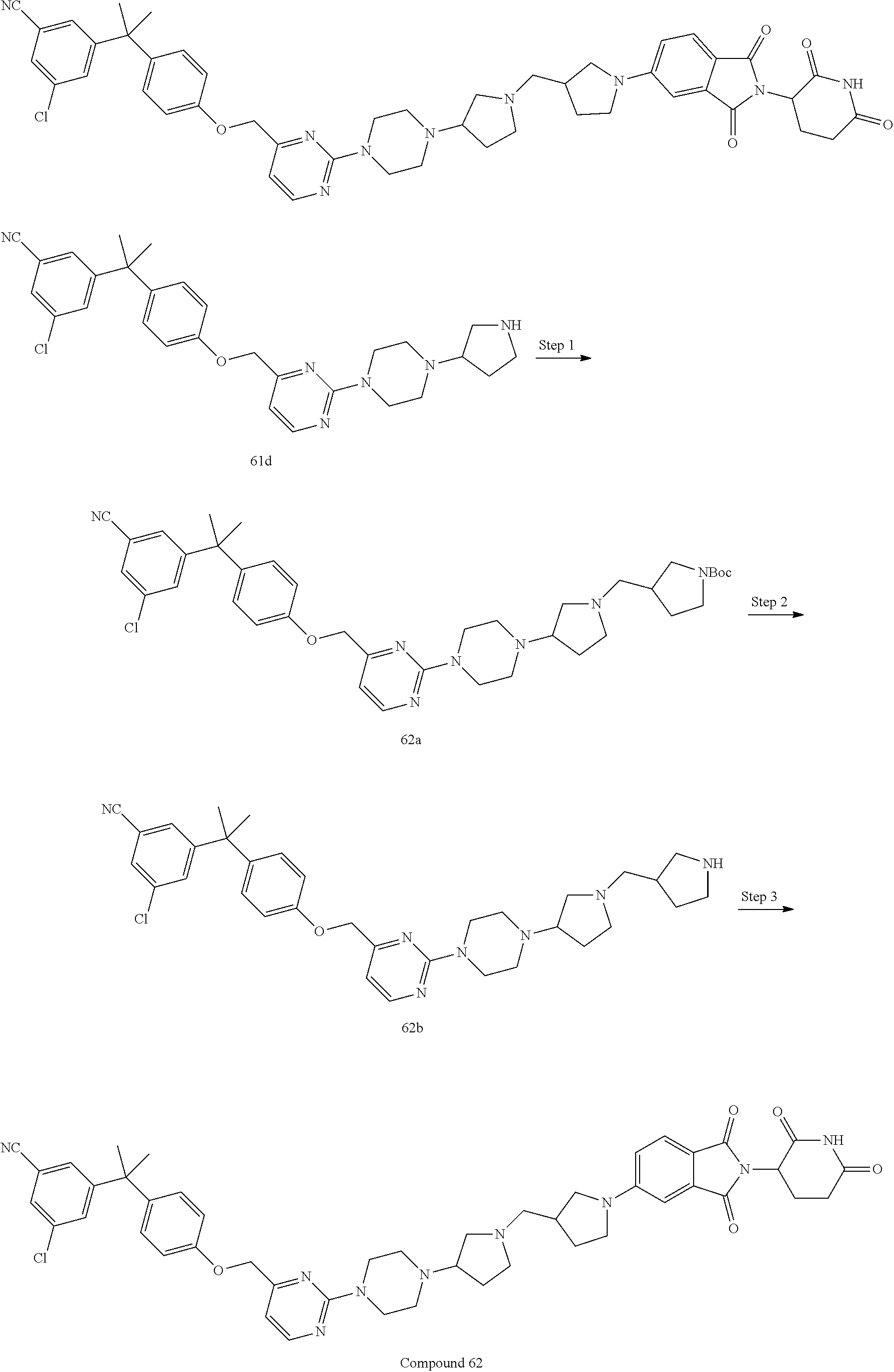 [SMILES: CC(C)(c1ccc(OCc2ccnc(N3CCN(C4CCN(CC5CCN(c6ccc7c(c6)C(=O)N(C6CCC(=O)NC6=O)C7=O)C5)C4)CC3)n2)cc1)c1cc(Cl)cc(C#N)c1 ; CC(C)(c1ccc(OCc2ccnc(N3CCN(C4CCNC4)CC3)n2)cc1)c1cc(Cl)cc(C#N)c1 ; CC(C)(C)OC(=O)N1CCC(CN2CCC(N3CCN(c4nccc(COc5ccc(C(C)(C)c6cc(Cl)cc(C#N)c6)cc5)n4)CC3)C2)C1 ; CC(C)(c1ccc(OCc2ccnc(N3CCN(C4CCN(CC5CCNC5)C4)CC3)n2)cc1)c1cc(Cl)cc(C#N)c1 ; CC(C)(c1ccc(OCc2ccnc(N3CCN(C4CCN(CC5CCN(c6ccc7c(c6)C(=O)N(C6CCC(=O)NC6=O)C7=O)C5)C4)CC3)n2)cc1)c1cc(Cl)cc(C#N)c1]

### Step 1: tert-butyl 3-((3-(4-(4-((4-(2-(3-chloro-5-cyanophenyl)propan-2-yl)phenoxy)methyl)pyrimidin-2-yl)piperazin-1-yl)pyrrolidin-1-yl)methyl)pyrrolidine-1-carboxylate (62a)

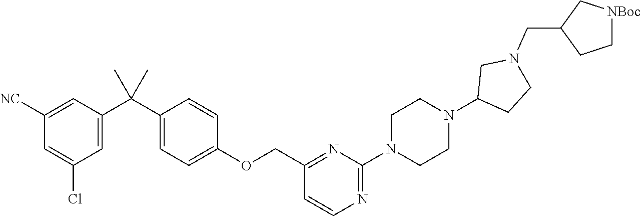 [SMILES: CC(C)(C)OC(=O)N1CCC(CN2CCC(N3CCN(c4nccc(COc5ccc(C(C)(C)c6cc(Cl)cc(C#N)c6)cc5)n4)CC3)C2)C1]

The above crude 61d (90 mg) was added to 10 mL of 1,2-dichloroethane, and tert-butyl 3-formylpyrrolidine-1-carboxylate (40 mg, 0.2 mmol) was added. The mixture was reacted at room temperature for 1 h, and then sodium triacetoxyborohydride (42 mg, 0.2 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 62a (110 mg, two-step yield from compound 61c: 75%).

LCMS m/z=700.4 [M+1]⁺

### Step 2: 3-chloro-5-(2-(4-((2-(4-(1-(pyrrolidin-3-ylmethyl)pyrrolidin-3-yl)piperazin-1-yl)pyrimidin-4-yl)methoxy)phenyl)propan-2-yl)benzonitrile (62b)

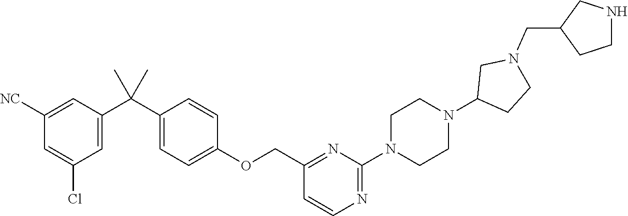 [SMILES: CC(C)(c1ccc(OCc2ccnc(N3CCN(C4CCN(CC5CCNC5)C4)CC3)n2)cc1)c1cc(Cl)cc(C#N)c1]

62a (110 mg, 0.16 mmol) was dissolved in 6 mL of dichloromethane, and 2 mL of trifluoroacetic acid was added. The mixture was reacted at room temperature for 2 h. The reaction liquid was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 62b (108 mg).

### Step 3: 3-chloro-5-(2-(4-((2-(4-(1-((1-(2-(2,6-dioxopiperidin-3-yl)-1,3-dioxoisoindolin-5-yl)pyrrolidin-3-yl)methyl)pyrrolidin-3-yl)piperazin-1-yl)pyrimidin-4-yl)methoxy)phenyl) propan-2-yl)benzonitrile (Compound 62) trifluoroacetate

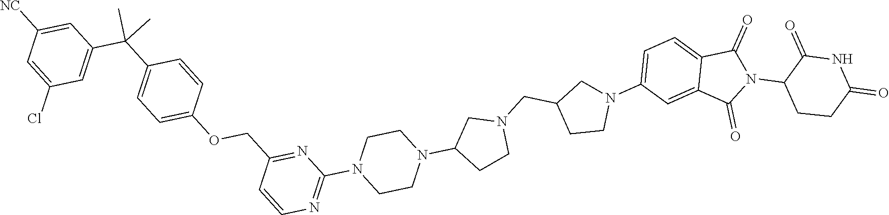 [SMILES: CC(C)(c1ccc(OCc2ccnc(N3CCN(C4CCN(CC5CCN(c6ccc7c(c6)C(=O)N(C6CCC(=O)NC6=O)C7=O)C5)C4)CC3)n2)cc1)c1cc(Cl)cc(C#N)c1]

The above crude 62b (108 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 62 trifluoroacetate (30 mg).

¹H NMR (400 MHz, DMSO-d₆) δ 11.05 (s, 1H), 8.44 (d, 1H), 7.88-7.81 (m, 1H), 7.72-7.62 (m, 2H), 7.56-7.50 (m, 1H), 7.21-7.13 (m, 2H), 6.99-6.91 (m, 3H), 6.87-6.78 (m, 2H), 5.11-4.97 (m, 3H), 4.00-3.76 (m, 9H), 3.75-3.65 (m, 1H), 3.62-3.50 (m, 1H), 3.50-3.29 (m, 3H), 3.27-3.17 (m, 1H), 3.17-2.96 (m, 4H), 2.96-2.68 (m, 2H), 2.65-2.51 (m, 2H), 2.41-2.10 (m, 3H), 2.10-1.98 (m, 1H), 1.92-1.75 (m, 1H), 1.64 (s, 6H).

LCMS m/z=428.8 [M/2+1]⁺

### Example 63: 3-chloro-5-(2-(4-((2-(4-(1-((1-(2-(2,6-dioxopiperidin-3-yl)-1,3-dioxoisoindolin-5-yl)piperidin-4-yl)methyl)pyrrolidin-3-yl)piperazin-1-yl)pyrimidin-4-yl)methoxy)phenyl)propan-2-yl)benzonitrile (Compound 63) trifluoroacetate

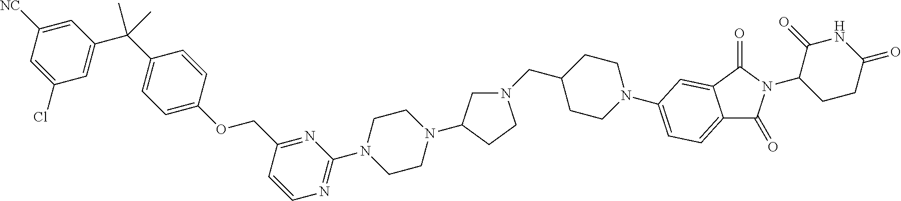 [SMILES: CC(C)(c1ccc(OCc2ccnc(N3CCN(C4CCN(CC5CCN(c6ccc7c(c6)C(=O)N(C6CCC(=O)NC6=O)C7=O)CC5)C4)CC3)n2)cc1)c1cc(Cl)cc(C#N)c1]

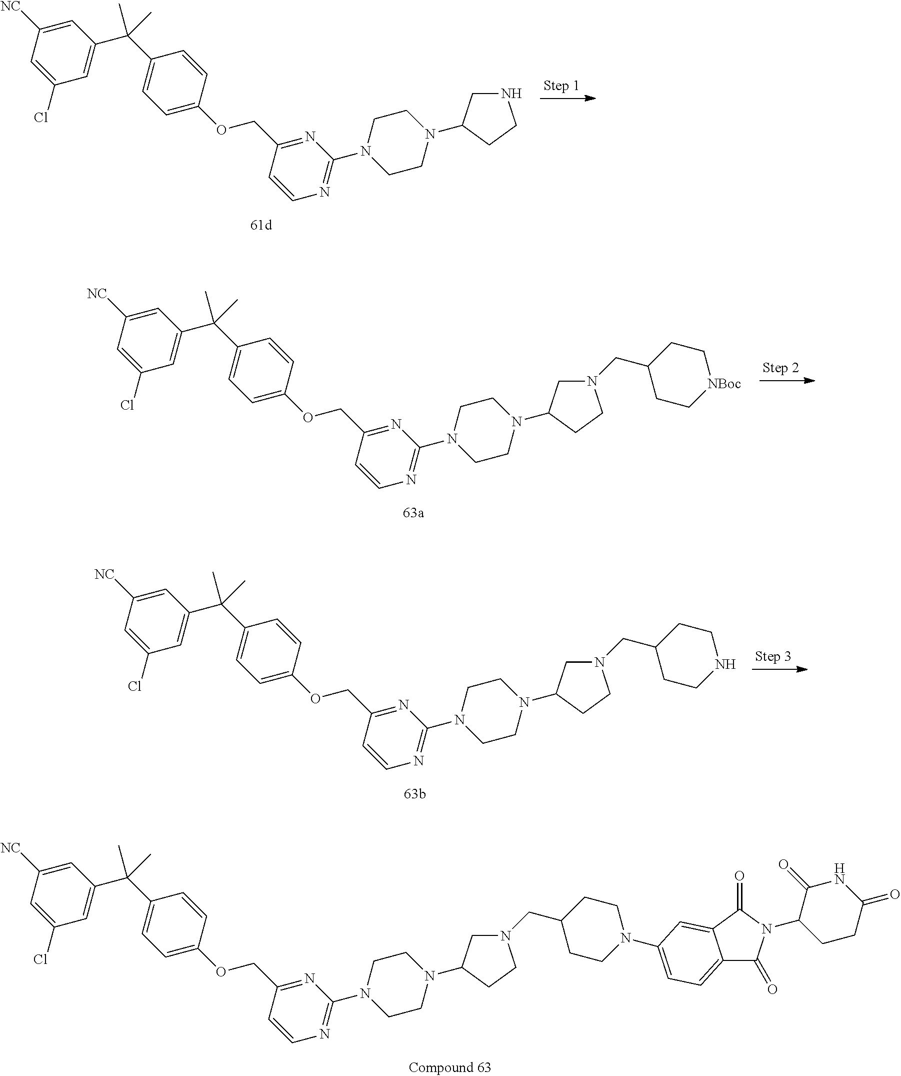 [SMILES: CC(C)(c1ccc(OCc2ccnc(N3CCN(C4CCNC4)CC3)n2)cc1)c1cc(Cl)cc(C#N)c1 ; CC(C)(C)OC(=O)N1CCC(CN2CCC(N3CCN(c4nccc(COc5ccc(C(C)(C)c6cc(Cl)cc(C#N)c6)cc5)n4)CC3)C2)CC1 ; CC(C)(c1ccc(OCc2ccnc(N3CCN(C4CCN(CC5CCNCC5)C4)CC3)n2)cc1)c1cc(Cl)cc(C#N)c1 ; CC(C)(c1ccc(OCc2ccnc(N3CCN(C4CCN(CC5CCN(c6ccc7c(c6)C(=O)N(C6CCC(=O)NC6=O)C7=O)CC5)C4)CC3)n2)cc1)c1cc(Cl)cc(C#N)c1]

### Step 1: tert-butyl 4-((3-(4-(4-((4-(2-(3-chloro-5-cyanophenyl)propan-2-yl)phenoxy)methyl)pyrimidin-2-yl)piperazin-1-yl)pyrrolidin-1-yl)methyl)piperidine-1-carboxylate (63a)

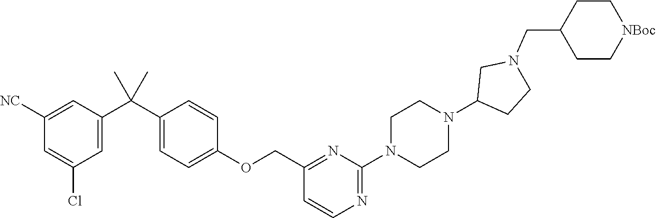 [SMILES: CC(C)(C)OC(=O)N1CCC(CN2CCC(N3CCN(c4nccc(COc5ccc(C(C)(C)c6cc(Cl)cc(C#N)c6)cc5)n4)CC3)C2)CC1]

The above crude 61d (90 mg) was added to 10 mL of 1,2-dichloroethane, and tert-butyl 4-formylpiperidine-1-carboxylate (43 mg, 0.2 mmol) was added. The mixture was reacted at room temperature for 1 h, and then sodium triacetoxyborohydride (42 mg, 0.2 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 63a (100 mg, two-step yield from compound 61c: 67%).

LCMS m/z=714.4 [M+1]⁺

### Step 2: 3-chloro-5-(2-(4-((2-(4-(1-(piperidin-4-ylmethyl)pyrrolidin-3-yl)piperazin-1-yl)pyrimidin-4-yl)methoxy)phenyl)propan-2-yl)benzonitrile (63b)

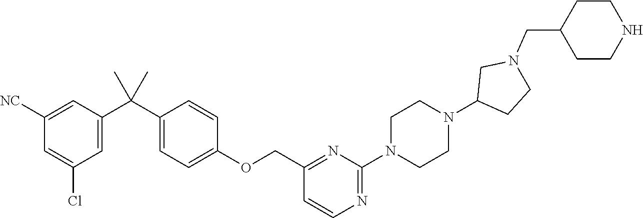 [SMILES: CC(C)(c1ccc(OCc2ccnc(N3CCN(C4CCN(CC5CCNCC5)C4)CC3)n2)cc1)c1cc(Cl)cc(C#N)c1]

63a (100 mg, 0.14 mmol) was dissolved in 6 mL of dichloromethane, and 2 mL of trifluoroacetic acid was added. The mixture was reacted at room temperature for 2 h. The reaction liquid was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 63b (98 mg).

### Step 3: 3-chloro-5-(2-(4-((2-(4-(1-((1-(2-(2,6-dioxopiperidin-3-yl)-1,3-dioxoisoindolin-5-yl)piperidin-4-yl)methyl)pyrrolidin-3-yl)piperazin-1-yl)pyrimidin-4-yl)methoxy)phenyl) propan-2-yl)benzonitrile (Compound 63) trifluoroacetate

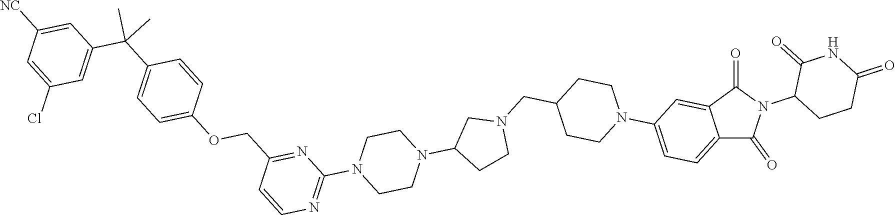 [SMILES: CC(C)(c1ccc(OCc2ccnc(N3CCN(C4CCN(CC5CCN(c6ccc7c(c6)C(=O)N(C6CCC(=O)NC6=O)C7=O)CC5)C4)CC3)n2)cc1)c1cc(Cl)cc(C#N)c1]

The above crude 63b (98 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 63 trifluoroacetate (18 mg).

¹H NMR (400 MHz, DMSO-d₆) δ 11.06 (s, 1H), 8.43 (d, 1H), 7.90-7.77 (m, 1H), 7.72-7.59 (m, 2H), 7.55-7.47 (m, 1H), 7.38-7.32 (m, 1H), 7.31-7.23 (m, 1H), 7.21-7.13 (m, 2H), 6.99-6.90 (m, 2H), 6.84-6.77 (m, 1H), 5.13-4.93 (m, 3H), 4.17-4.04 (m, 2H), 4.00-3.81 (m, 4H), 3.50-3.35 (m, 5H), 3.19-2.80 (m, 9H), 2.65-2.51 (m, 2H), 2.36-2.16 (m, 2H), 2.12-1.96 (m, 2H), 1.90-1.78 (m, 2H), 1.64 (s, 6H), 1.40-1.15 (m, 2H).

LCMS m/z=435.8 [M/2+1]⁺

### Example 64

### 3-chloro-5-(2-(4-((2-(4-(3-(4-(2-(2,6-dioxopiperidin-3-yl)-1,3-dioxoisoindolin-5-yl)piperazin-1-yl)azetidin-1-yl)piperidin-1-yl)pyrimidin-4-yl)methoxy)phenyl)propan-2-yl)benzonitrile (Compound 64) trifluoroacetate

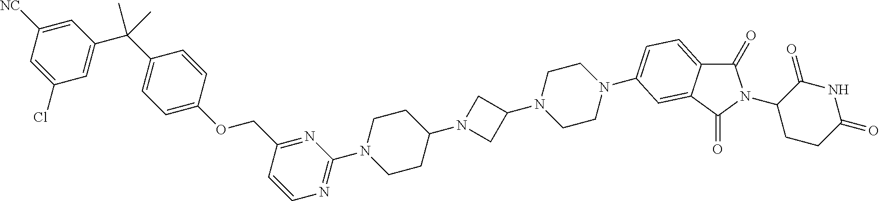 [SMILES: CC(C)(c1ccc(OCc2ccnc(N3CCC(N4CC(N5CCN(c6ccc7c(c6)C(=O)N(C6CCC(=O)NC6=O)C7=O)CC5)C4)CC3)n2)cc1)c1cc(Cl)cc(C#N)c1]

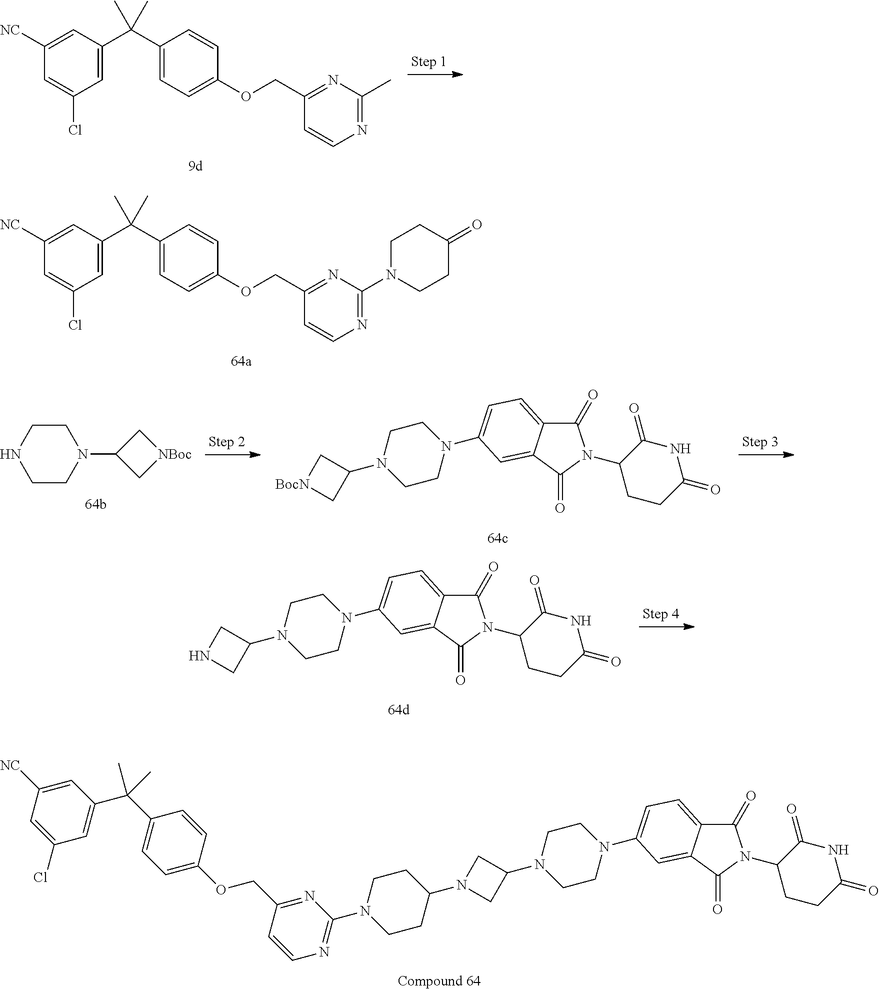 [SMILES: Cc1nccc(COc2ccc(C(C)(C)c3cc(Cl)cc(C#N)c3)cc2)n1 ; CC(C)(c1ccc(OCc2ccnc(N3CCC(=O)CC3)n2)cc1)c1cc(Cl)cc(C#N)c1 ; CC(C)(C)OC(=O)N1CC(N2CCNCC2)C1 ; CC(C)(C)OC(=O)N1CC(N2CCN(c3ccc4c(c3)C(=O)N(C3CCC(=O)NC3=O)C4=O)CC2)C1 ; O=C1CCC(N2C(=O)c3ccc(N4CCN(C5CNC5)CC4)cc3C2=O)C(=O)N1 ; CC(C)(c1ccc(OCc2ccnc(N3CCC(N4CC(N5CCN(c6ccc7c(c6)C(=O)N(C6CCC(=O)NC6=O)C7=O)CC5)C4)CC3)n2)cc1)c1cc(Cl)cc(C#N)c1]

### Step 1: 3-chloro-5-(2-(4-((2-(4-oxopiperidin-1-yl)pyrimidin-4-yl)methoxy)phenyl)propan-2-yl)benzonitrile (64a)

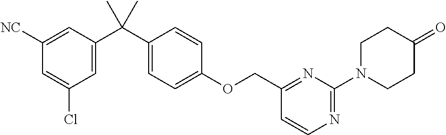 [SMILES: CC(C)(c1ccc(OCc2ccnc(N3CCC(=O)CC3)n2)cc1)c1cc(Cl)cc(C#N)c1]

3-chloro-5-(2-(4-((2-chloropyrimidin-4-yl)methoxy)phenyl)propan-2-yl)benzonitrile (9d) (550 mg, 1.38 mmol) was dissolved in 15 mL of DMF, and piperidine-4-one (200 mg, 2.02 mmol) and solid potassium bicarbonate (414 mg, 4.14 mmol) were successively added. The mixture was warmed to 70° C. and stirred for 2 h. The reaction liquid was cooled to room temperature, and 100 mL of water was added. The mixture was extracted twice with 50 mL of ethyl acetate, and the organic phase was washed with 100 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 64a (300 mg, yield from compound 9d: 47%).

LCMS m/z=461.2 [M+1]⁺

### Step 2: tert-butyl 3-(4-(2-(2,6-dioxopiperidin-3-yl)-1,3-dioxoisoindolin-5-yl)piperazin-1-yl)azetidine-1-carboxylate (64c)

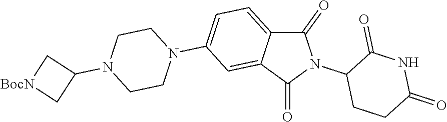 [SMILES: CC(C)(C)OC(=O)N1CC(N2CCN(c3ccc4c(c3)C(=O)N(C3CCC(=O)NC3=O)C4=O)CC2)C1]

Tert-butyl 3-(piperazin-1-yl)azetidine-1-carboxylate (64b) (see WO 2019195609 for the synthetic method) (350 mg, 1.45 mmol) was dissolved in 10 mL of DMSO, and 0.5 mL of DIPEA and 2-(2,6-dioxopiperidin-3-yl)-5-fluoroisoindoline-1,3-dione (see WO 2017197056 for the synthetic method) (552 mg, 2.0 mmol) were added. The reaction was stirred at 85° C. for 3 h. The reaction liquid was cooled to room temperature, and 200 mL of water was added to precipitate out a solid. The solid was filtered by suction, and the filter cake was dissolved with 200 mL of dichloromethane, dried over anhydrous sodium sulfate, and concentrated under reduced pressure. The crude product was separated and purified by silica gel column chromatography (dichloromethane/methanol (v/v)=15:1), to afford 64c (300 mg, yield: 42%).

### Step 3: 5-(4-(azetidin-3-yl)piperazin-1-yl)-2-(2,6-dioxopiperidin-3-yl)isoindoline-1,3-dione (64d)

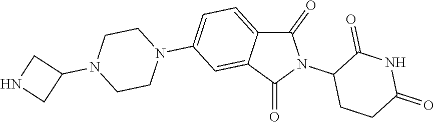 [SMILES: O=C1CCC(N2C(=O)c3ccc(N4CCN(C5CNC5)CC4)cc3C2=O)C(=O)N1]

64c (300 mg, 0.60 mmol) was dissolved in 8 mL of dichloromethane, and 2 mL of trifluoroacetic acid was added. The mixture was reacted at room temperature for 2 h. The reaction system was concentrated under reduced pressure, and 30 mL of dichloromethane was added. The mixture was adjusted to pH 9.0 with a saturated sodium bicarbonate solution, and the aqueous phase was extracted with 30 mL of dichloromethane, dried over anhydrous sodium sulfate, and concentrated under reduced pressure, to afford crude 64d (258 mg).

LCMS m/z=398.2 [M+1]⁺

### Step 4: 3-chloro-5-(2-(4-((2-(4-(3-(4-(2-(2,6-dioxopiperidin-3-yl)-1,3-dioxoisoindolin-5-yl)piperazin-1-yl)azetidin-1-yl)piperidin-1-yl)pyrimidin-4-yl)methoxy)phenyl)propan-2-yl)benzonitrile (Compound 64) trifluoroacetate

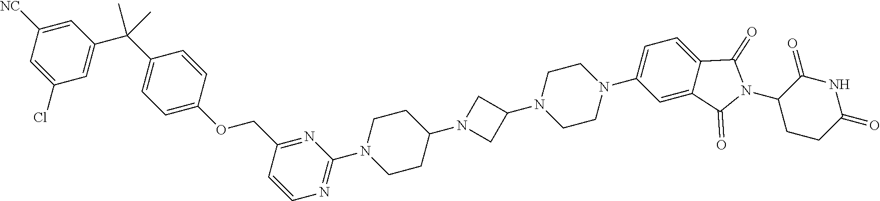 [SMILES: CC(C)(c1ccc(OCc2ccnc(N3CCC(N4CC(N5CCN(c6ccc7c(c6)C(=O)N(C6CCC(=O)NC6=O)C7=O)CC5)C4)CC3)n2)cc1)c1cc(Cl)cc(C#N)c1]

64a (100 mg, 0.22 mmol) and the above crude 64d (258 mg) were added to a mixed solvent of 20 mL of 1,2-dichloroethane and 2 mL of DMSO, respectively, and 1 mL of triethylamine was added. The mixture was reacted at room temperature for 2 h, and then sodium triacetoxyborohydride (63 mg, 0.3 mmol) was added. The resulting mixture was reacted at room temperature for 12 h. To the reaction liquid were added 50 mL of dichloromethane and 50 mL of 1 mol/L aqueous sodium hydroxide solution. The organic phase was separated, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 64 trifluoroacetate (35 mg).

¹H NMR (400 MHz, DMSO-d₆) δ 11.07 (s, 1H), 8.39 (d, 1H), 7.86-7.81 (m, 1H), 7.72 (d, 1H), 7.67-7.62 (m, 1H), 7.55-7.50 (m, 1H), 7.44-7.38 (m, 1H), 7.35-7.28 (m, 1H), 7.21-7.13 (m, 2H), 6.98-6.91 (m, 2H), 6.75 (d, 1H), 5.08 (dd, 1H), 4.99 (s, 2H), 4.82-4.69 (m, 2H), 4.32-4.11 (m, 4H), 3.58-3.38 (m, 5H), 2.98-2.80 (m, 3H), 2.73-2.51 (m, 7H), 2.10-1.93 (m, 3H), 1.73-1.55 (m, 6H), 1.36-1.19 (m, 2H).

LCMS m/z=842.5 [M+1]⁺

### Example 65

### 3-chloro-5-(2-(4-((2-(4-(3-((4-(2-(2,6-dioxopiperidin-3-yl)-1,3-dioxoisoindolin-4-yl)piperazin-1-yl)methyl)azetidin-1-yl)piperidin-1-yl)pyrimidin-4-yl)methoxy)phenyl) propan-2-yl)benzonitrile (Compound 65) trifluoroacetate

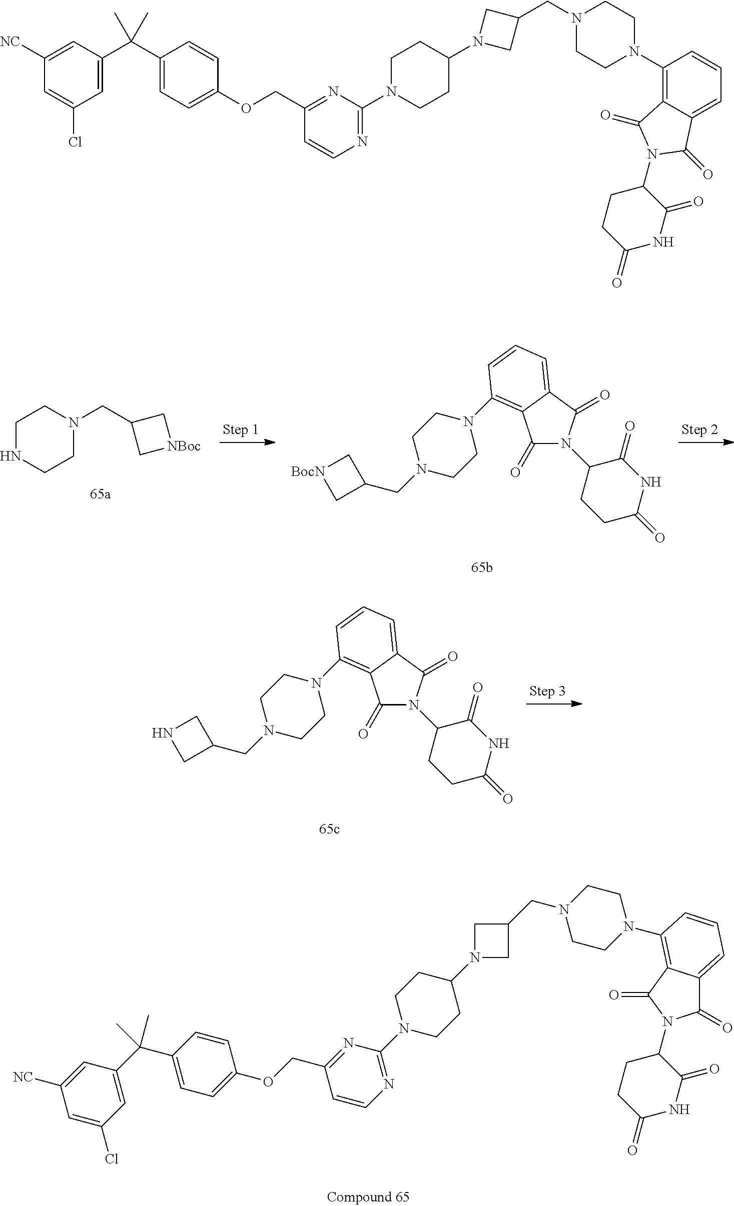 [SMILES: CC(C)(c1ccc(OCc2ccnc(N3CCC(N4CC(CN5CCN(c6cccc7c6C(=O)N(C6CCC(=O)NC6=O)C7=O)CC5)C4)CC3)n2)cc1)c1cc(Cl)cc(C#N)c1 ; CC(C)(C)OC(=O)N1CC(CN2CCNCC2)C1 ; CC(C)(C)OC(=O)N1CC(CN2CCN(c3cccc4c3C(=O)N(C3CCC(=O)NC3=O)C4=O)CC2)C1 ; O=C1CCC(N2C(=O)c3cccc(N4CCN(CC5CNC5)CC4)c3C2=O)C(=O)N1 ; CC(C)(c1ccc(OCc2ccnc(N3CCC(N4CC(CN5CCN(c6cccc7c6C(=O)N(C6CCC(=O)NC6=O)C7=O)CC5)C4)CC3)n2)cc1)c1cc(Cl)cc(C#N)c1]

### Step 1: tert-butyl 3-((4-(2-(2,6-dioxopiperidin-3-yl)-1,3-dioxoisoindolin-4-yl)piperazin-1-yl)methyl)azetidine-1-carboxylate (65b)

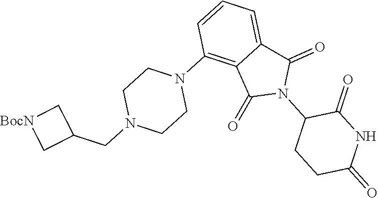 [SMILES: CC(C)(C)OC(=O)N1CC(CN2CCN(c3cccc4c3C(=O)N(C3CCC(=O)NC3=O)C4=O)CC2)C1]

Tert-butyl 3-(piperazin-1-ylmethyl)azetidine-1-carboxylate (65a) (see WO 2018071606 for the synthetic method) (400 mg, 1.57 mmol) was dissolved in 10 mL of DMSO, and 0.5 mL of DIPEA and 2-(2,6-dioxopiperidin-3-yl)-4-fluoroisoindoline-1,3-dione (see WO 2017197056 for the synthetic method) (552 mg, 2.0 mmol) were added. The reaction was stirred at 85° C. for 3 h. The reaction liquid was cooled to room temperature, and 200 mL of water was added to precipitate out a solid. The solid was filtered by suction, and the filter cake was dissolved with 200 mL of dichloromethane, dried over anhydrous sodium sulfate, and concentrated under reduced pressure. The crude product was separated and purified by silica gel column chromatography (dichloromethane/methanol (v/v)=15:1), to afford 65b (250 mg, yield: 31%).

LCMS m/z=512.3 [M+1]⁺

### Step 2: 4-(4-(azetidin-3-ylmethyl)piperazin-1-yl)-2-(2,6-dioxopiperidin-3-yl)isoindoline-1,3-dione (65c)

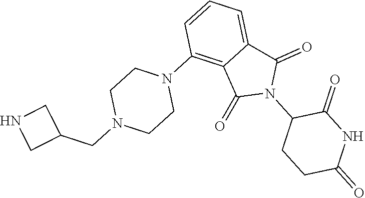 [SMILES: O=C1CCC(N2C(=O)c3cccc(N4CCN(CC5CNC5)CC4)c3C2=O)C(=O)N1]

65b (250 mg, 0.49 mmol) was dissolved in 8 mL of dichloromethane, and 2 mL of trifluoroacetic acid was added. The mixture was stirred at room temperature for 2 h. The reaction system was concentrated under reduced pressure, and 30 mL of dichloromethane was added. The mixture was adjusted to pH 9.0 with a saturated sodium bicarbonate solution, and the aqueous phase was extracted with 30 mL of dichloromethane, dried over anhydrous sodium sulfate, and concentrated under reduced pressure, to afford crude 65c (192 mg).

### Step 3: 3-chloro-5-(2-(4-((2-(4-(3-((4-(2-(2,6-dioxopiperidin-3-yl)-1,3-dioxoisoindolin-4-yl)piperazin-1-yl)methyl)azetidin-1-yl)piperidin-1-yl)pyrimidin-4-yl)methoxy)phenyl) propan-2-yl)benzonitrile (Compound 65) trifluoroacetate

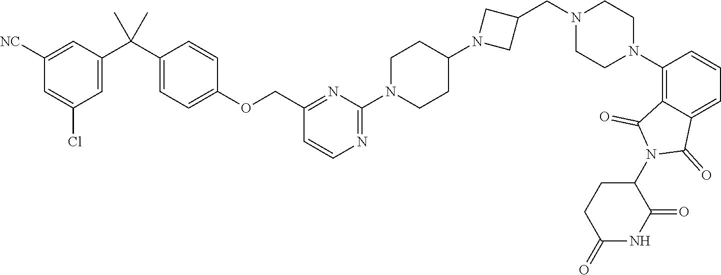 [SMILES: CC(C)(c1ccc(OCc2ccnc(N3CCC(N4CC(CN5CCN(c6cccc7c6C(=O)N(C6CCC(=O)NC6=O)C7=O)CC5)C4)CC3)n2)cc1)c1cc(Cl)cc(C#N)c1]

64a (100 mg, 0.22 mmol) and the above crude 65c (192 mg) were added to a mixed solvent of 20 mL of dichloroethane and 2 mL of DMSO, respectively, and 1 mL of triethylamine was added. The mixture was reacted at ambient temperature for 2 h, and sodium triacetoxyborohydride (63 mg, 0.3 mmol) was added. The resulting mixture was reacted at room temperature for 12 h. To the reaction liquid were added 50 mL of dichloromethane and 50 mL of 1 mol/L aqueous sodium hydroxide solution. The organic phase was separated, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 65 trifluoroacetate (30 mg).

1H NMR (400 MHz, DMSO-d6) δ 11.09 (s, 1H), 8.40 (d, 1H), 7.86-7.82 (m, 1H), 7.82-7.75 (m, 1H), 7.67-7.62 (m, 1H), 7.55-7.51 (m, 1H), 7.49 (d, 1H), 7.43 (d, 1H), 7.21-7.13 (m, 2H), 6.99-6.91 (m, 2H), 6.75 (d, 1H), 5.11 (dd, 1H), 4.99 (s, 2H), 4.82-4.70 (m, 2H), 4.42-4.00 (m, 4H), 3.80-3.20 (m, 12H), 2.99-2.82 (m, 3H), 2.68-2.45 (m, 2H), 2.12-1.92 (m, 3H), 1.64 (s, 6H), 1.36-1.19 (m, 2H).

LCMS m/z=856.4 [M+1]⁺

### Example 66

### 3-chloro-5-(2-(4-((2-(4-((1-(7-(2-(2,6-dioxopiperidin-3-yl)-1,3-dioxoisoindolin-5-yl)-7-azaspiro[3.5]nonan-2-yl)pyrrolidin-3-yl)methyl)piperazin-1-yl)pyrimidin-4-yl)methoxy)phenyl)propan-2-yl)benzonitrile (Compound 66) trifluoroacetate

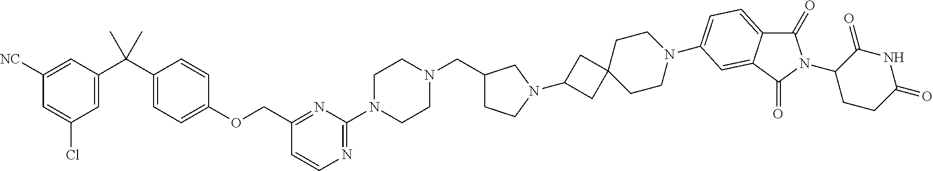 [SMILES: CC(C)(c1ccc(OCc2ccnc(N3CCN(CC4CCN(C5CC6(CCN(c7ccc8c(c7)C(=O)N(C7CCC(=O)NC7=O)C8=O)CC6)C5)C4)CC3)n2)cc1)c1cc(Cl)cc(C#N)c1]

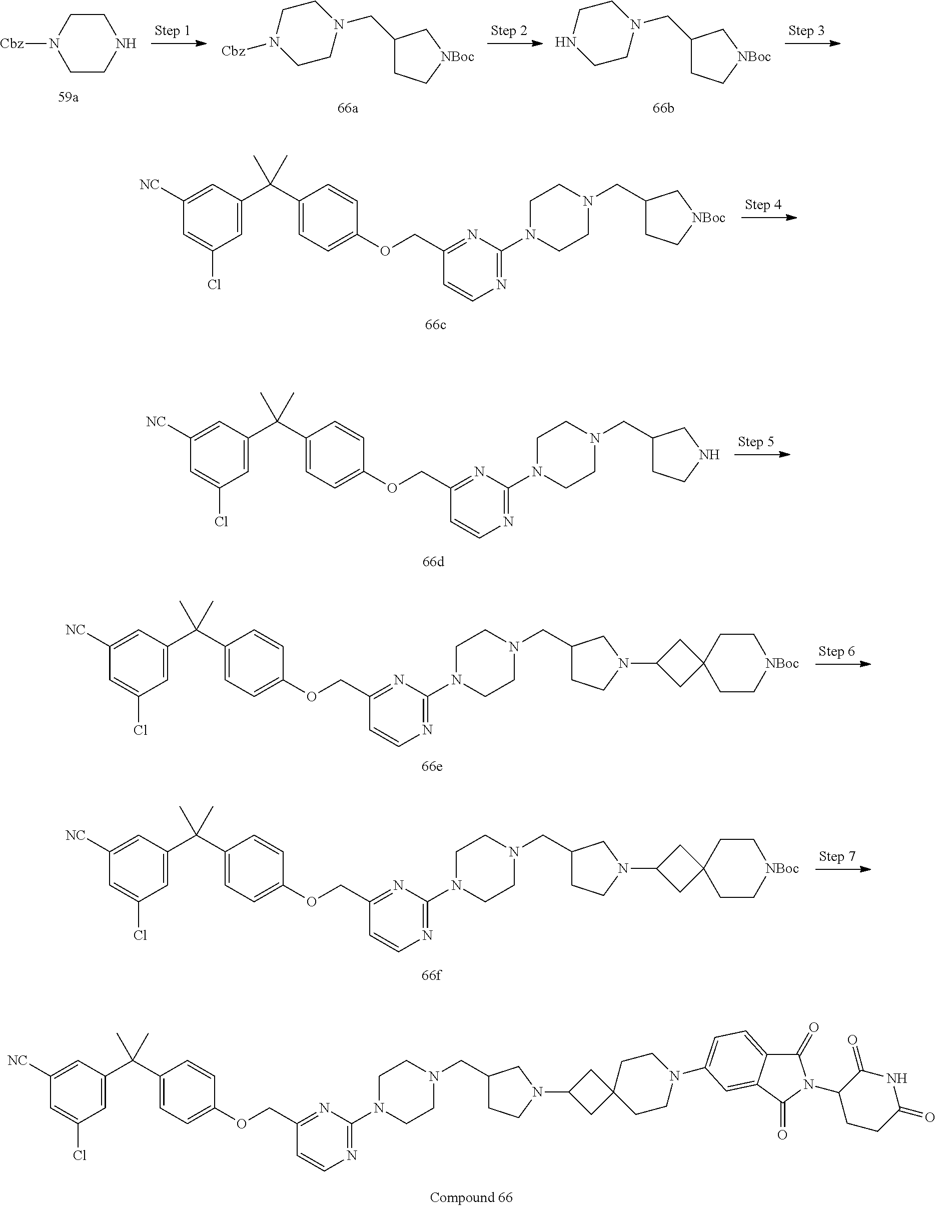

### Step 1: benzyl 4-((1-(tert-butoxycarbonyl)pyrrolidin-3-yl)methyl)piperazine-1-carboxylate (66a)

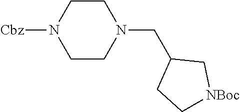 [SMILES: CC(C)(C)OC(=O)N1CCC(CN2CCN(C(=O)OCc3ccccc3)CC2)C1]

Benzyl piperazine-1-carboxylate (59a) (8.8 g, 40 mmol) and tert-butyl 3-formylpyrrolidine-1-carboxylate (8.0 g, 40 mmol) were added to 100 mL of dichloromethane, and the mixture was reacted at room temperature for 2 h. Sodium triacetoxyborohydride (8.4 g, 40 mmol) was then added, and the mixture was reacted at room temperature for 12 h. To the reaction liquid were added 100 mL of dichloromethane and 100 mL of 1 mol/L aqueous sodium hydroxide solution. The organic phase was separated, dried over anhydrous sodium sulfate, and concentrated under reduced pressure. The crude product was separated and purified by silica gel column chromatography (ethyl acetate/petroleum ether (v/v)=1: 10-1:1), to afford 66a (15.5 g, yield: 96%).

LCMS m/z=404.2 [M+1]⁺

### Step 2: tert-butyl 3-(piperazin-1-ylmethyl)pyrrolidine-1-carboxylate (66b)

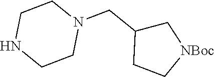 [SMILES: CC(C)(C)OC(=O)N1CCC(CN2CCNCC2)C1]

66a (15.5 g, 38.4 mmol) was dissolved in methanol (200 mL), and 2 g of 10% palladium on carbon was added. The mixture was reacted at room temperature under hydrogen atmosphere for 16 h. The reaction system was filtered, and the filtrate was concentrated under reduced pressure, to afford crude 66b (10.0 g).

LCMS m/z=270.3 [M+1]⁺

### Step 3: tert-butyl 3-((4-(4-((4-(2-(3-chloro-5-cyanophenyl)propan-2-yl)phenoxy)methyl)pyrimidin-2-yl)piperazin-1-yl)methyl)pyrrolidine-1-carboxylate (66c)

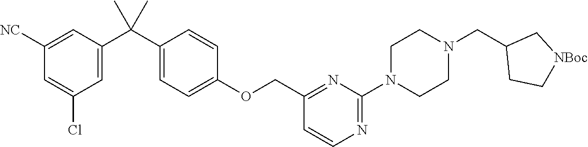 [SMILES: CC(C)(C)OC(=O)N1CCC(CN2CCN(c3nccc(COc4ccc(C(C)(C)c5cc(Cl)cc(C#N)c5)cc4)n3)CC2)C1]

3-chloro-5-(2-(4-((2-chloropyrimidin-4-yl)methoxy)phenyl)propan-2-yl)benzonitrile (9d) (660 mg, 1.66 mmol) was dissolved in 15 mL of DMF, and the above crude 66b (540 mg) and solid potassium bicarbonate (414 mg, 4.14 mmol) were successively added. The mixture was warmed to 75° C. and reacted for 4 h. The reaction liquid was cooled to room temperature, and 60 mL of water was added. The mixture was extracted twice with 60 mL of ethyl acetate, and the organic phase was washed with 60 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 66c (720 mg, yield from compound 9d: 69%).

LCMS m/z=631.1 [M+1]⁺

### Step 4: 3-chloro-5-(2-(4-((2-(4-(pyrrolidin-3-ylmethyl)piperazin-1-yl)pyrimidin-4-yl)methoxy)phenyl)propan-2-yl)benzonitrile (66d)

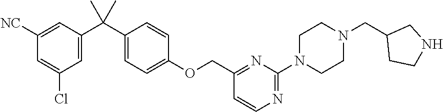 [SMILES: CC(C)(c1ccc(OCc2ccnc(N3CCN(CC4CCNC4)CC3)n2)cc1)c1cc(Cl)cc(C#N)c1]

66c (720 mg, 1.14 mmol) was dissolved in 30 mL of dichloromethane, and 10 mL of trifluoroacetic acid was added. The mixture was reacted at room temperature for 2 h. The reaction system was concentrated under reduced pressure, and 100 mL of dichloromethane was added. The mixture was adjusted to pH 9.0 with a saturated sodium bicarbonate solution, and the aqueous phase was extracted with 50 mL of dichloromethane. The organic phases were combined, dried over anhydrous sodium sulfate, and concentrated under reduced pressure, to afford crude 66d (550 mg).

LCMS m/z=531.3 [M+1]⁺

### Step 5: tert-butyl 2-(3-((4-(4-((4-(2-(3-chloro-5-cyanophenyl)propan-2-yl)phenoxy)methyl)pyrimidin-2-yl)piperazin-1-yl)methyl)pyrrolidin-1-yl)-7-azaspiro[3.5]nonane-7-carboxylate (66e)

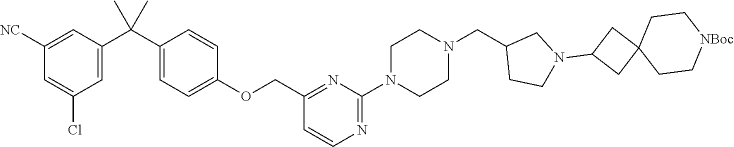 [SMILES: CC(C)(C)OC(=O)N1CCC2(CC1)CC(N1CCC(CN3CCN(c4nccc(COc5ccc(C(C)(C)c6cc(Cl)cc(C#N)c6)cc5)n4)CC3)C1)C2]

The above crude 66d (90 mg) was added to 10 mL of 1,2-dichloroethane, and tert-butyl 2-oxo-7-azaspiro[3.5]nonane-7-carboxylate (72 mg, 0.3 mmol) was added. The mixture was reacted at room temperature for 1 h, and then sodium triacetoxyborohydride (63 mg, 0.3 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 66e (80 mg, two-step yield from compound 66c: 57%).

LCMS m/z=754.4 [M+1]⁺

### Step 6: 3-(2-(4-((2-(4-((1-(7-azaspiro[3.5]nonan-2-yl)pyrrolidin-3-yl)methyl)piperazin-1-yl)pyrimidin-4-yl)methoxy)phenyl)propan-2-yl)-5-chlorobenzonitrile (66f)

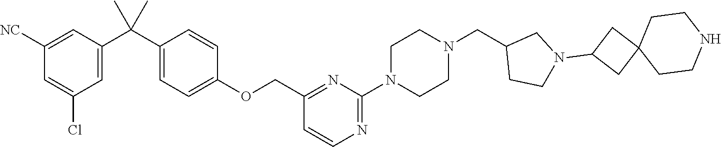 [SMILES: CC(C)(c1ccc(OCc2ccnc(N3CCN(CC4CCN(C5CC6(CCNCC6)C5)C4)CC3)n2)cc1)c1cc(Cl)cc(C#N)c1]

66e (80 mg, 0.11 mmol) was dissolved in 6 mL of dichloromethane, and 2 mL of trifluoroacetic acid was added. The mixture was reacted at room temperature for 2 h. The reaction liquid was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 66f (75 mg).

Step 7: 3-chloro-5-(2-(4-((2-(4-((1-(7-(2-(2,6-dioxopiperidin-3-yl)-1,3-dioxoisoindolin-5-yl)-7-azaspiro[3.5]nonan-2-yl)pyrrolidin-3-yl)methyl)piperazin-1-yl)pyrimidin-4-yl)methoxy)phenyl)propan-2-yl)benzonitrile (Compound 66) trifluoroacetate

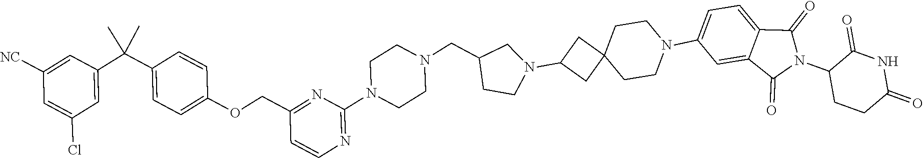 [SMILES: CC(C)(c1ccc(OCc2ccnc(N3CCN(CC4CCN(C5CC6(CCN(c7ccc8c(c7)C(=O)N(C7CCC(=O)NC7=O)C8=O)CC6)C5)C4)CC3)n2)cc1)c1cc(Cl)cc(C#N)c1]

The above crude 66f (75 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 66 trifluoroacetate (30 mg).

¹H NMR (400 MHz, DMSO-d₆) δ 11.06 (s, 1H), 8.47 (d, 1H), 7.87-7.81 (m, 1H), 7.70-7.61 (m, 2H), 7.55-7.50 (m, 1H), 7.40-7.30 (m, 1H), 7.30-7.22 (m, 1H), 7.22-7.12 (m, 2H), 7.00-6.90 (m, 2H), 6.86 (d, 1H), 5.13-4.95 (m, 3H), 3.96-3.60 (m, 6H), 3.48-3.36 (m, 4H), 3.36-3.16 (m, 5H), 3.16-3.03 (m, 1H), 3.03-2.70 (m, 3H), 2.66-2.50 (m, 6H), 2.32-2.14 (m, 2H), 2.07-1.93 (m, 3H), 1.76-1.52 (m, 10H).

LCMS m/z=455.9 [M/2+1]³⁰

### Example 67

### 3-chloro-5-(2-(4-((2-(4-((1-(1-(2-(2,6-dioxopiperidin-3-yl)-1,3-dioxoisoindolin-5-yl)piperidin-3-yl)pyrrolidin-3-yl)methyl)piperazin-1-yl)pyrimidin-4-yl)methoxy)phenyl) propan-2-yl)benzonitrile (Compound 67) trifluoroacetate

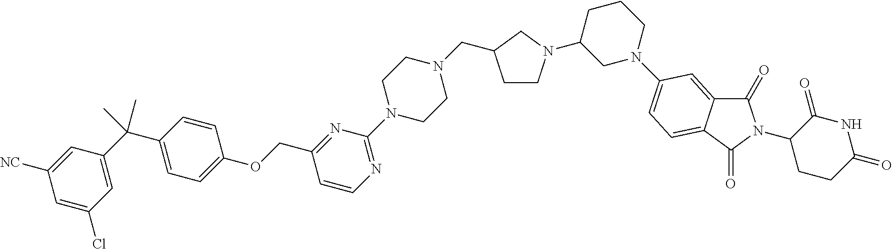 [SMILES: CC(C)(c1ccc(OCc2ccnc(N3CCN(CC4CCN(C5CCCN(c6ccc7c(c6)C(=O)N(C6CCC(=O)NC6=O)C7=O)C5)C4)CC3)n2)cc1)c1cc(Cl)cc(C#N)c1]

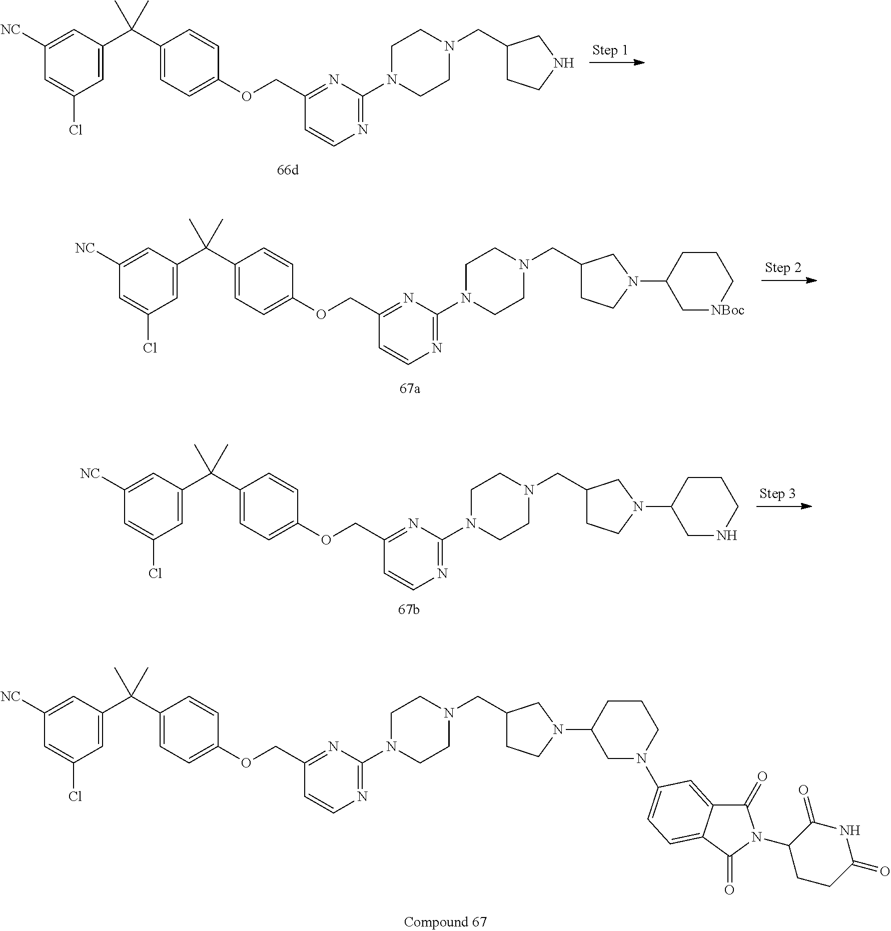 [SMILES: CC(C)(c1ccc(OCc2ccnc(N3CCN(CC4CCNC4)CC3)n2)cc1)c1cc(Cl)cc(C#N)c1 ; CC(C)(C)OC(=O)N1CCCC(N2CCC(CN3CCN(c4nccc(COc5ccc(C(C)(C)c6cc(Cl)cc(C#N)c6)cc5)n4)CC3)C2)C1 ; CC(C)(c1ccc(OCc2ccnc(N3CCN(CC4CCN(C5CCCNC5)C4)CC3)n2)cc1)c1cc(Cl)cc(C#N)c1 ; CC(C)(c1ccc(OCc2ccnc(N3CCN(CC4CCN(C5CCCN(c6ccc7c(c6)C(=O)N(C6CCC(=O)NC6=O)C7=O)C5)C4)CC3)n2)cc1)c1cc(Cl)cc(C#N)c1]

### Step 1: tert-butyl 3-(3-((4-(4-((4-(2-(3-chloro-5-cyanophenyl)propan-2-yl)phenoxy)methyl)pyrimidin-2-yl)piperazin-1-yl)methyl)pyrrolidin-1-yl)piperidine-1-carboxylate (67a)

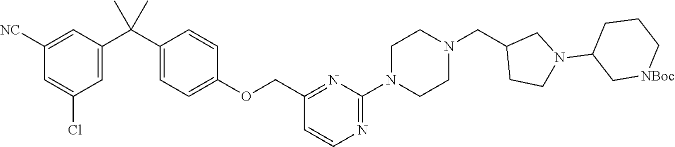 [SMILES: CC(C)(C)OC(=O)N1CCCC(N2CCC(CN3CCN(c4nccc(COc5ccc(C(C)(C)c6cc(Cl)cc(C#N)c6)cc5)n4)CC3)C2)C1]

The above crude 66d (90 mg) was added to 10 mL of 1,2-dichloroethane, and tert-butyl 3-oxopiperidine-1-carboxylate (60 mg, 0.3 mmol) was added. The mixture was reacted at room temperature for 1 h, and then sodium triacetoxyborohydride (63 mg, 0.3 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 67a (70 mg, two-step yield from compound 66c: 53%).

LCMS m/z=714.3 [M+1]⁺

### Step 2: 3-chloro-5-(2-(4-((2-(4-((1-(piperidin-3-yl)pyrrolidin-3-yl)methyl)piperazin-1-yl)pyrimidin-4-yl)methoxy)phenyl)propan-2-yl)benzonitrile (67b)

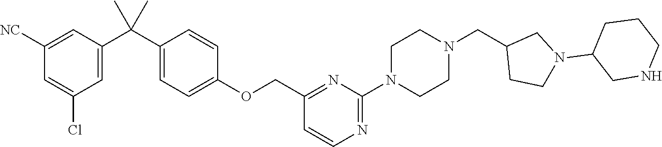 [SMILES: CC(C)(c1ccc(OCc2ccnc(N3CCN(CC4CCN(C5CCCNC5)C4)CC3)n2)cc1)c1cc(Cl)cc(C#N)c1]

67a (70 mg, 0.098 mmol) was dissolved in 6 mL of dichloromethane, and 2 mL of trifluoroacetic acid was added. The mixture was reacted at room temperature for 2 h. The reaction liquid was concentrated under reduced pressure, and 30 mL of dichloromethane was added. The mixture was adjusted to pH 9.0 with a saturated sodium bicarbonate solution. The organic phase was separated, dried over anhydrous sodium sulfate, and concentrated under reduced pressure, to afford crude 67b (59 mg).

### Step 3: 3-chloro-5-(2-(4-((2-(4-((1-(1-(2-(2,6-dioxopiperidin-3-yl)-1,3-dioxoisoindolin-5-yl)piperidin-3-yl)pyrrolidin-3-yl)methyl)piperazin-1-yl)pyrimidin-4-yl)methoxy)phenyl) propan-2-yl)benzonitrile (Compound 67) trifluoroacetate

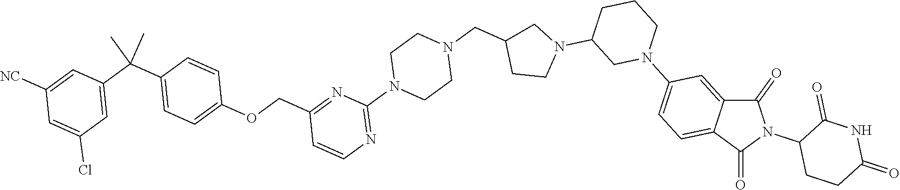 [SMILES: CC(C)(c1ccc(OCc2ccnc(N3CCN(CC4CCN(C5CCCN(c6ccc7c(c6)C(=O)N(C6CCC(=O)NC6=O)C7=O)C5)C4)CC3)n2)cc1)c1cc(Cl)cc(C#N)c1]

The above crude 67b (59 mg) was dissolved in 5 mL of DMSO, and 0.2 mL of DIPEA and 2-(2,6-dioxopiperidin-3-yl)-5-fluoroisoindoline-1,3-dione (see WO 2017197056 for the synthetic method) (50 mg, 0.18 mmol) were added. The reaction was stirred at 85° C. for 3 h. The reaction liquid was cooled to room temperature, and diluted with 50 mL of ethyl acetate. The organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 67 trifluoroacetate (15 mg).

¹H NMR (400 MHz, DMSO-d₆) δ 11.07 (s, 1H), 8.44 (d, 1H), 7.87-7.82 (m, 1H), 7.73 (d, 1H), 7.68-7.62 (m, 1H), 7.55-7.51 (m, 1H), 7.49-7.43 (m, 1H), 7.40-7.33 (m, 1H), 7.22-7.12 (m, 2H), 7.00-6.90 (m, 2H), 6.88-6.77 (m, 1H), 5.14-5.04 (m, 1H), 5.01 (s, 2H), 4.25-4.10 (m, 1H), 3.97-3.80 (m, 1H), 3.45-2.70 (m, 20H), 2.67-2.52 (m, 2H), 2.45-2.10 (m, 2H), 2.09-1.96 (m, 1H), 1.95-1.81 (m, 1H), 1.80-1.50 (m, 8H).

LCMS m/z=435.7 [M/2+1]⁺

### Example 68

### 3-chloro-5-(2-(4-((2-(9-((1-(2-(2,6-dioxopiperidin-3-yl)-1,3-dioxoisoindolin-5-yl)piperidin-4-yl)methyl)-3,9-diazaspiro[5.5]undecan-3-yl)pyrimidin-4-yl)methoxy)phenyl) propan-2-yl)benzonitrile (compound 68)

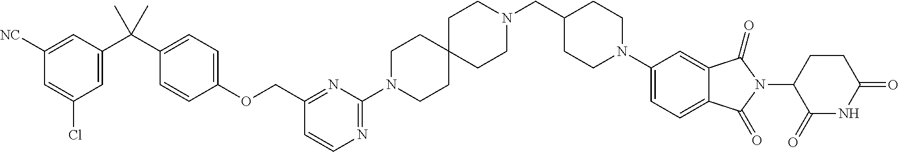 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)cc(C#N)c1]

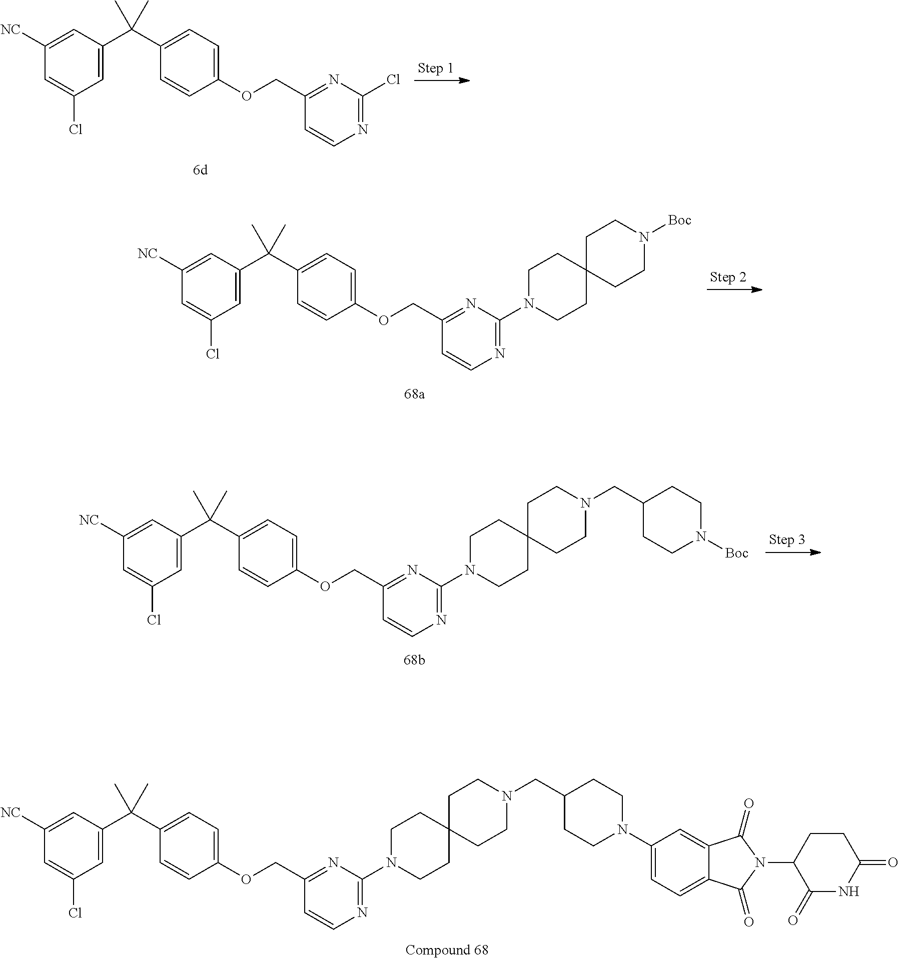 [SMILES: CC(C)(c1ccc(OCc2ccnc(Cl)n2)cc1)c1cc(Cl)cc(C#N)c1 ; CC(C)(C)OC(=O)N1CCC2(CC1)CCN(c1nccc(COc3ccc(C(C)(C)c4cc(Cl)cc(C#N)c4)cc3)n1)CC2 ; CC(C)(C)OC(=O)N1CCC(CN2CCC3(CC2)CCN(c2nccc(COc4ccc(C(C)(C)c5cc(Cl)cc(C#N)c5)cc4)n2)CC3)CC1 ; CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)cc(C#N)c1]

### Step 1: tert-butyl 9-(4-((4-(2-(3-chloro-5-cyanophenyl)propan-2-yl)phenoxy)methyl)pyrimidin-2-yl)-3,9-diazaspiro[5.5]undecane-3-carboxylate (68a)

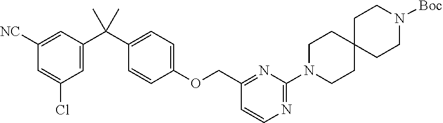 [SMILES: CC(C)(C)OC(=O)N1CCC2(CC1)CCN(c1nccc(COc3ccc(C(C)(C)c4cc(Cl)cc(C#N)c4)cc3)n1)CC2]

3-chloro-5-(2-(4-((2-chloropyrimidin-4-yl)methoxy)phenyl)propan-2-yl)benzonitrile (9d) (1.7 g, 4.28 mmol) was dissolved in 20 mL of DMF, and tert-butyl 3,9-diazaspiro[5.5]undecane-3-carboxylate (1.1 g, 4.32 mmol) and solid potassium bicarbonate (0.87 g, 8.70 mmol) were successively added. The mixture was warmed to 75° C. and reacted for 4 h. The reaction liquid was cooled to room temperature, and 30 mL of water was added. The mixture was extracted twice with 60 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (petroleum ether/ethyl acetate (v/v)=1:1), to afford 68a (2.1 g, yield: 80%).

### Step 2: tert-butyl 4-((9-(4-((4-(2-(3-chloro-5-cyanophenyl)propan-2-yl)phenoxy)methyl)pyrimidin-2-yl)-3,9-diazaspiro[5.5]undecan-3-yl)methyl)piperidine-1-carboxylate (68b)

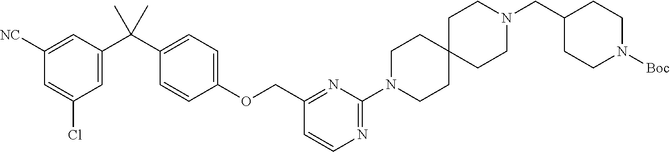 [SMILES: CC(C)(C)OC(=O)N1CCC(CN2CCC3(CC2)CCN(c2nccc(COc4ccc(C(C)(C)c5cc(Cl)cc(C#N)c5)cc4)n2)CC3)CC1]

68a (400 mg, 0.65 mmol) was dissolved in 6 mL of dichloromethane, and 4 mL of trifluoroacetic acid was added. The mixture was reacted at room temperature for 2 h. The reaction system was concentrated under reduced pressure, and 10 mL of tetrahydrofuran and solid sodium bicarbonate (160 mg, 1.90 mmol) were added. The mixture was stirred at room temperature for 0.5 h, and then tert-butyl 4-formylpiperidine-1-carboxylate (280 mg, 1.31 mmol) was added. The mixture was stirred at room temperature for 0.5 h, and then sodium triacetoxyborohydride (690 mg, 3.26 mmol) was added. The resulting mixture was reacted at room temperature for 16 h. To the reaction liquid was slowly added 50 mL of saturated sodium bicarbonate solution, and the mixture was extracted with 100 mL of dichloromethane. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 68b (312 mg, yield: 67%).

### Step 3: 3-chloro-5-(2-(4-((2-(9-((1-(2-(2,6-dioxopiperidin-3-yl)-1,3-dioxoisoindolin-5-yl)piperidin-4-yl)methyl)-3,9-diazaspiro[5.5]undecan-3-yl)pyrimidin-4-yl)methoxy)phenyl) propan-2-yl)benzonitrile (Compound 68)

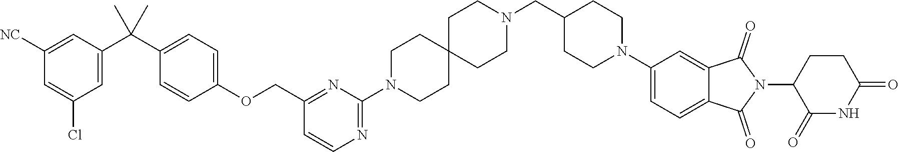 [SMILES: CC(C)(c1ccc(OCc2ccnc(N3CCC4(CCN(CC5CCN(c6ccc7c(c6)C(=O)N(C6CCC(=O)NC6=O)C7=O)CC5)CC4)CC3)n2)cc1)c1cc(Cl)cc(C#N)c1]

68b (240 mg, 0.34 mmol) was dissolved in 5 mL of DCM, and 2 mL of trifluoroacetic acid was added. The mixture was stirred at room temperature for 2 h and concentrated under reduced pressure to afford an oil. The oil was dissolved in 8 mL of DMSO, and solid sodium bicarbonate (110 mg, 1.31 mmol) was added. The mixture was stirred at room temperature for 0.5 h, and then 0.28 mL of DIPEA and 2-(2,6-dioxopiperidin-3-yl)-5-fluoroisoindoline-1,3-dione (see WO 2017197056 for the synthetic method) (140 mg, 0.51 mmol) were added. The resulting mixture was warmed to 85° C. and reacted for 3 h. The reaction liquid was cooled to room temperature, and 20 mL of water was added to precipitate out a large number of yellow solids. The yellow solids were filtered by suction, dissolved in 50 mL of mixed solvent (dichloromethane/methanol (v/v)=10:1), dried over anhydrous sodium sulfate, and concentrated under reduced pressure. The crude product was then separated and purified by silica gel column chromatography (dichloromethane/methanol (v/v)=1:0-9:1), to afford compound 68 (85 mg, yield from compound 68b: 29%).

¹H NMR (400 MHz, DMSO-d₆) δ 11.05 (s, 1H), 8.32 (d, 1H), 7.86-7.81 (m, 1H), 7.68-7.61 (m, 2H), 7.54-7.49 (m, 1H), 7.33-7.27 (m, 1H), 7.22 (dd, 1H), 7.19-7.12 (m, 2H), 6.97-6.90 (m, 2H), 6.64 (d, 1H), 5.06 (dd, 1H), 4.96 (s, 2H), 4.10-3.94 (m, 2H), 3.80-3.62 (m, 4H), 3.04-2.80 (m, 3H), 2.70-2.51 (m, 2H), 2.46-2.24 (m, 4H), 2.23-2.07 (m, 2H), 2.07-1.93 (m, 1H), 1.90-1.72 (m, 3H), 1.64 (s, 6H), 1.57-1.45 (m, 4H), 1.45-1.36 (m, 4H), 1.20-1.06 (m, 2H).

LCMS m/z=869.3 [M+1]⁺

### Example 69

### 3-chloro-5-(2-(4-((2-(7-((1-(2-(2,6-dioxopiperidin-3-yl)-1,3-dioxoisoindolin-5-yl)piperidin-4-yl)methyl)-2,7-diazaspiro[3.5]nonan-2-yl)pyrimidin-4-yl)methoxy)phenyl) propan-2-yl)benzonitrile (Compound 69) trifluoroacetate

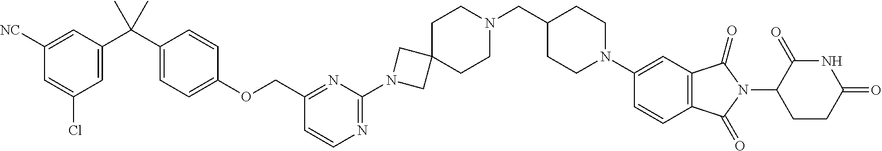 [SMILES: CC(C)(c1ccc(OCc2ccnc(N3CC4(CCN(CC5CCN(c6ccc7c(c6)C(=O)N(C6CCC(=O)NC6=O)C7=O)CC5)CC4)C3)n2)cc1)c1cc(Cl)cc(C#N)c1]

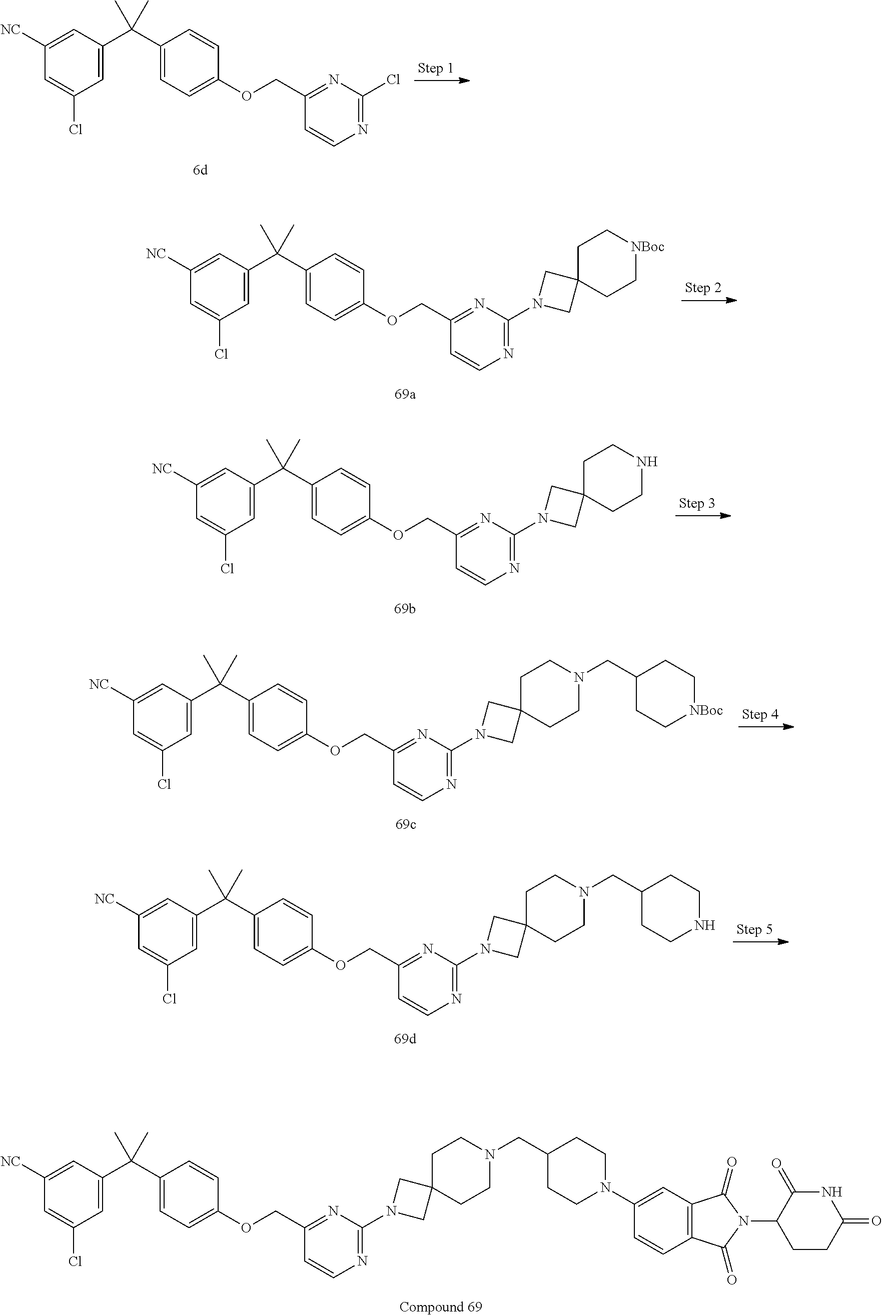 [SMILES: CC(C)(c1ccc(OCc2ccnc(Cl)n2)cc1)c1cc(Cl)cc(C#N)c1 ; CC(C)(C)OC(=O)N1CCC2(CC1)CN(c1nccc(COc3ccc(C(C)(C)c4cc(Cl)cc(C#N)c4)cc3)n1)C2 ; CC(C)(c1ccc(OCc2ccnc(N3CC4(CCNCC4)C3)n2)cc1)c1cc(Cl)cc(C#N)c1 ; CC(C)(C)OC(=O)N1CCC(CN2CCC3(CC2)CN(c2nccc(COc4ccc(C(C)(C)c5cc(Cl)cc(C#N)c5)cc4)n2)C3)CC1 ; CC(C)(c1ccc(OCc2ccnc(N3CC4(CCN(CC5CCNCC5)CC4)C3)n2)cc1)c1cc(Cl)cc(C#N)c1 ; CC(C)(c1ccc(OCc2ccnc(N3CC4(CCN(CC5CCN(c6ccc7c(c6)C(=O)N(C6CCC(=O)NC6=O)C7=O)CC5)CC4)C3)n2)cc1)c1cc(Cl)cc(C#N)c1]

Step 1: tert-butyl-2-(4-((4-(2-(3-chloro-5-cyanophenyl)propan-2-yl)phenoxy)methyl)pyrimidin-2-yl)-2,7-diazaspiro [3,5]nonane-7-carboxyate (69a)

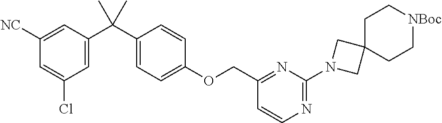 [SMILES: CC(C)(C)OC(=O)N1CCC2(CC1)CN(c1nccc(COc3ccc(C(C)(C)c4cc(Cl)cc(C#N)c4)cc3)n1)C2]

3-chloro-5-(2-(4-((2-chloropyrimidin-4-yl)methoxy)phenyl)propan-2-yl)benzonitrile (9d) (0.25 g, 0.63 mmol) was dissolved in 5 mL of N, N-dimethylformamide, and tert-butyl 2,7-diazaspiro[3.5]nonane-7-carboxylate (0.22 g, 0.97 mmol) and solid potassium bicarbonate (0.16 g, 1.60 mmol) were successively added. The mixture was warmed to 60° C. and reacted for 2 h. The reaction liquid was cooled to room temperature, and 30 mL of water was added. The mixture was extracted with ethyl acetate (30 mL×3), and the organic phase was washed with 100 mL of saturated sodium chloride solution, dried over anhydrous sodium sulfate, and concentrated under reduced pressure. The crude product was separated and purified by silica gel column chromatography (dichloromethane/methanol (v/v)=99:1), to afford 69a (0.26 g, yield: 70%).

### Step 2: 3-(2-(4-((2-(2,7-diazaspiro[3.5]nonan-2-yl)pyrimidin-4-yl)methoxy)phenyl)propan-2-yl)-5-chlorobenzonitrile (69b) trifluoroacetate

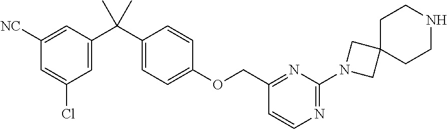 [SMILES: CC(C)(c1ccc(OCc2ccnc(N3CC4(CCNCC4)C3)n2)cc1)c1cc(Cl)cc(C#N)c1]

69a (0.26 g, 0.44 mmol) was dissolved in 2 mL of dichloromethane, and trifluoroacetic acid (3.06 g, 26.84 mmol) was added. The mixture was stirred at room temperature for 2 h. The reaction system was concentrated under reduced pressure, to afford crude 69b trifluoroacetate (0.26 g).

### Step 3: tert-butyl 4-((2-(4-((4-(2-(3-chloro-5-cyanophenyl)propan-2-yl)phenoxy)methyl)pyrimidin-2-yl)-2,7-diazaspiro[3.5]nonan-7-yl)methyl)piperidine-1-carboxylate (69c)

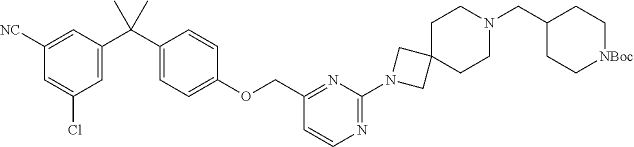 [SMILES: CC(C)(C)OC(=O)N1CCC(CN2CCC3(CC2)CN(c2nccc(COc4ccc(C(C)(C)c5cc(Cl)cc(C#N)c5)cc4)n2)C3)CC1]

The above crude 69b trifluoroacetate (0.12 g) was dissolved in 5 mL of dichloromethane, and solid sodium bicarbonate (0.11 g, 1.31 mmol) was added. The mixture was stirred at room temperature for 0.5 h, and then tert-butyl 4-formylpiperidine-1-carboxylate (0.064 g, 0.3 mmol) and sodium triacetoxyborohydride (0.11 g, 0.52 mmol) were added, respectively. The resulting mixture was reacted at room temperature for 16 h. To the reaction system was added 5 mL of saturated sodium bicarbonate solution. The mixture was extracted with ethyl acetate (10 mL×3), and the organic phase was washed with 20 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 69c (0.17 g, two-step yield from compound 69a: >99%).

LCMS m/z=685.3 [M+1]⁺

### Step 4: 3-chloro-5-(2-(4-((2-(7-(piperidin-4-ylmethyl)-2,7-diazaspiro[3.5]nonan-2-yl)pyrimidin-4-yl)methoxy)phenyl)propan-2-yl)benzonitrile (69d) trifluoroacetate

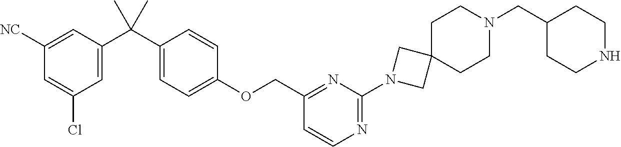 [SMILES: CC(C)(c1ccc(OCc2ccnc(N3CC4(CCN(CC5CCNCC5)CC4)C3)n2)cc1)c1cc(Cl)cc(C#N)c1]

69c (0.17 g, 0.25 mmol) was dissolved in 2 mL of dichloromethane, and trifluoroacetic acid (3.06 g, 26.84 mmol) was added. The mixture was reacted at room temperature for 2 h. The reaction system was concentrated under reduced pressure, to afford crude 69d trifluoroacetate (0.18 g).

### Step 5: 3-chloro-5-(2-(4-((2-(7-((1-(2-(2,6-dioxopiperidin-3-yl)-1,3-dioxoisoindolin-5-yl)piperidin-4-yl)methyl)-2,7-diazaspiro[3.5]nonan-2-yl)pyrimidin-4-yl)methoxy)phenyl) propan-2-yl)benzonitrile (Compound 69) trifluoroacetate

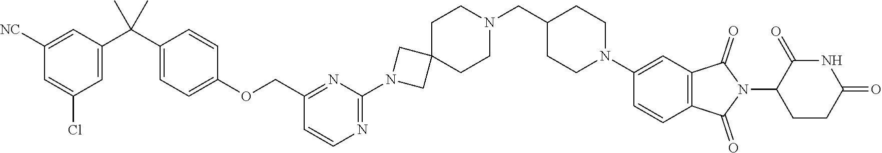 [SMILES: CC(C)(c1ccc(OCc2ccnc(N3CC4(CCN(CC5CCN(c6ccc7c(c6)C(=O)N(C6CCC(=O)NC6=O)C7=O)CC5)CC4)C3)n2)cc1)c1cc(Cl)cc(C#N)c1]

To a reaction flask were respectively added the crude 69d trifluoroacetate (0.18 g), 2-(2,6-dioxopiperidin-3-yl)-5-fluoroisoindoline-1,3-dione (see WO 2017197056 for the synthetic method) (0.20 g, 0.72 mmol) and diisopropylethylamine (0.3 mL). 5 mL of DMSO was added, and the mixture was warmed to 80° C. and reacted for 4 h. The reaction liquid was cooled to room temperature, and 20 mL of water was added. The mixture was extracted with dichloromethane (20 mL×3), and the organic phase was washed with 20 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 69 trifluoroacetate (58 mg).

¹H NMR (400 MHz, DMSO-d₆) δ 11.06 (s, 1H), 8.36 (d, 1H), 7.87-7.82 (m, 1H), 7.71-7.62 (m, 2H), 7.55-7.50 (m, 1H), 7.39-7.33 (m, 1H), 7.31-7.23 (m, 1H), 7.21-7.12 (m, 2H), 6.98-6.90 (m, 2H), 6.76 (d, 1H), 5.07 (dd, 1H), 4.96 (s, 2H), 4.15-4.03 (m, 2H), 3.88 (s, 2H), 3.81 (s, 2H), 3.55-3.40 (m, 2H), 3.10-2.80 (m, 7H), 2.70-2.51 (m, 2H), 2.23-1.90 (m, 6H), 1.90-1.77 (m, 2H), 1.64 (s, 6H), 1.37-1.20 (m, 2H).

LCMS m/z=841.3 [M+1]⁺.

### Example 70: 3-chloro-5-(2-(4-((2-(7-((1-(2-(2,6-dioxopiperidin-3-yl)-1,3-dioxoisoindolin-5-yl)azetidin-3-yl)methyl)-2,7-diazaspiro[3.5]nonan-2-yl)pyrimidin-4-yl)methoxy)phenyl) propan-2-yl)benzonitrile (Compound 70) trifluoroacetate

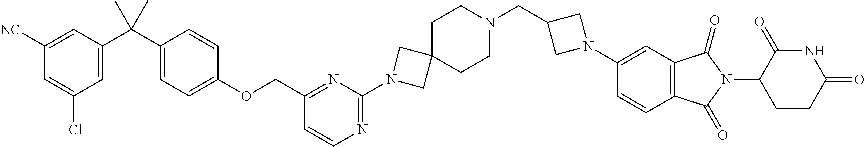 [SMILES: CC(C)(c1ccc(OCc2ccnc(N3CC4(CCN(CC5CN(c6ccc7c(c6)C(=O)N(C6CCC(=O)NC6=O)C7=O)C5)CC4)C3)n2)cc1)c1cc(Cl)cc(C#N)c1]

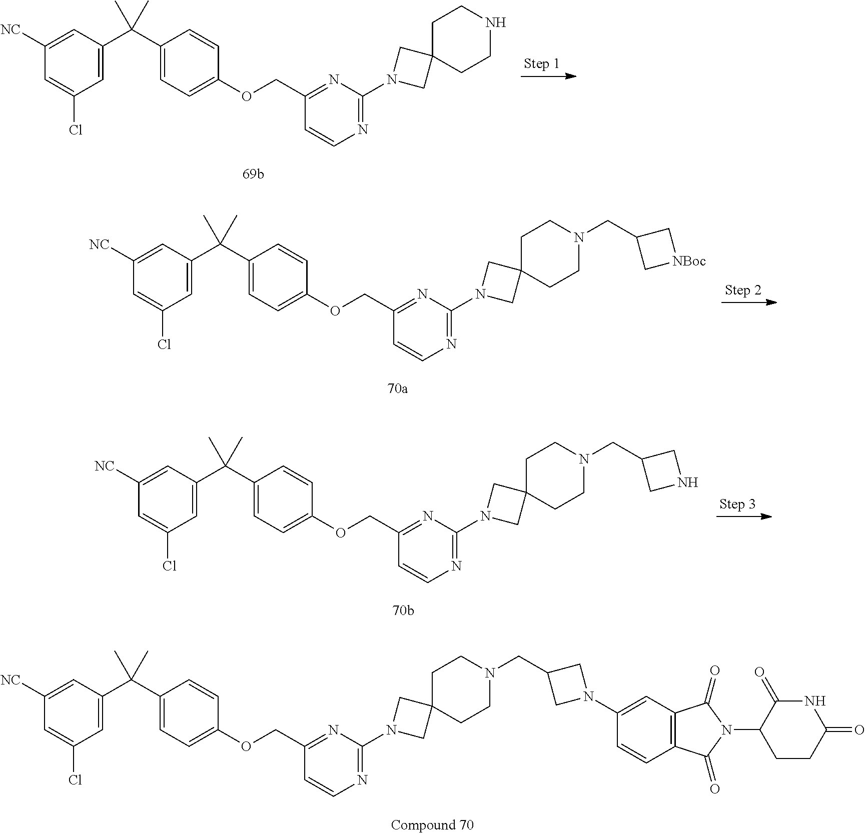 [SMILES: CC(C)(c1ccc(OCc2ccnc(N3CC4(CCNCC4)C3)n2)cc1)c1cc(Cl)cc(C#N)c1 ; CC(C)(C)OC(=O)N1CC(CN2CCC3(CC2)CN(c2nccc(COc4ccc(C(C)(C)c5cc(Cl)cc(C#N)c5)cc4)n2)C3)C1 ; CC(C)(c1ccc(OCc2ccnc(N3CC4(CCN(CC5CNC5)CC4)C3)n2)cc1)c1cc(Cl)cc(C#N)c1 ; CC(C)(c1ccc(OCc2ccnc(N3CC4(CCN(CC5CN(c6ccc7c(c6)C(=O)N(C6CCC(=O)NC6=O)C7=O)C5)CC4)C3)n2)cc1)c1cc(Cl)cc(C#N)c1]

### Step 1: tert-butyl 3-((2-(4-((4-(2-(3-chloro-5-cyanophenyl)propan-2-yl)phenoxy)methyl)pyrimidin-2-yl)-2,7-diazaspiro[3.5]nonan-7-yl)methyl)azetidine-1-carboxylate (70a)

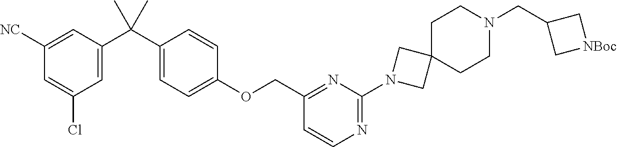 [SMILES: CC(C)(C)OC(=O)N1CC(CN2CCC3(CC2)CN(c2nccc(COc4ccc(C(C)(C)c5cc(Cl)cc(C#N)c5)cc4)n2)C3)C1]

The above crude 69b trifluoroacetate (0.12 g) was dissolved in 5 mL of dichloromethane, and solid sodium bicarbonate (0.11 g, 1.31 mmol) was added. The mixture was stirred at room temperature for 0.5 h, and then tert-butyl 3-formylazetidine-1-carboxylate (0.046 g, 0.25 mmol) and sodium triacetoxyborohydride (0.11 g, 0.52 mmol) were added, respectively. The resulting mixture was reacted at room temperature for 16 h. To the reaction system was added 5 mL of saturated sodium bicarbonate solution. The mixture was extracted with ethyl acetate (10 mL×3), and the organic phase was washed with 20 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 70a (0.16 g, two-step yield from compound 69a: >99%).

LCMS m/z=657.3 [M+1]⁺

### Step 2: 3-(2-(4-((2-(7-(azetidin-3-ylmethyl)-2,7-diazaspiro[3.5]nonan-2-yl)pyrimidin-4-yl)methoxy)phenyl)propan-2-yl)-5-chlorobenzonitrile (70b) trifluoroacetate

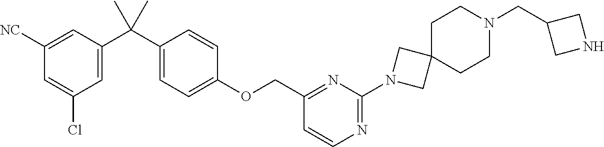 [SMILES: CC(C)(c1ccc(OCc2ccnc(N3CC4(CCN(CC5CNC5)CC4)C3)n2)cc1)c1cc(Cl)cc(C#N)c1]

70a (0.16 g, 0.24 mmol) was dissolved in 2 mL of dichloromethane, and trifluoroacetic acid (3.06 g, 26.84 mmol) was added. The mixture was reacted at room temperature for 2 h. The reaction system was concentrated under reduced pressure, to afford crude 70b trifluoroacetate (0.20 g).

### Step 3: 3-chloro-5-(2-(4-((2-(7-((1-(2-(2,6-dioxopiperidin-3-yl)-1,3-dioxoisoindolin-5-yl)azetidin-3-yl)methyl)-2,7-diazaspiro[3.5]nonan-2-yl)pyrimidin-4-yl)methoxy)phenyl) propan-2-yl)benzonitrile (Compound 70) trifluoroacetate

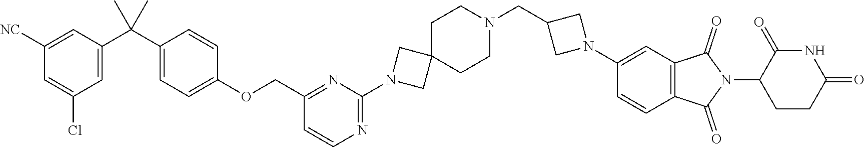 [SMILES: CC(C)(c1ccc(OCc2ccnc(N3CC4(CCN(CC5CN(c6ccc7c(c6)C(=O)N(C6CCC(=O)NC6=O)C7=O)C5)CC4)C3)n2)cc1)c1cc(Cl)cc(C#N)c1]

To a reaction flask were respectively added the above crude 70b trifluoroacetate (0.20 g), 2-(2,6-dioxopiperidin-3-yl)-5-fluoroisoindoline-1,3-dione (see WO 2017197056 for the synthetic method) (0.20 g, 0.72 mmol) and diisopropylethylamine (0.3 mL). 5 mL of DMSO was added, and the mixture was warmed to 80° C. and reacted for 4 h. The reaction liquid was cooled to room temperature, and 20 mL of water was added. The mixture was extracted with dichloromethane (20 mL×3), and the organic phase was washed with 20 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 70 trifluoroacetate (35 mg).

¹H NMR (400 MHz, DMSO-d₆) δ 11.05 (s, 1H), 8.36 (d, 1H), 7.88-7.80 (m, 1H), 7.72-7.62 (m, 2H), 7.56-7.48 (m, 1H), 7.21-7.13 (m, 2H), 6.98-6.88 (m, 2H), 6.84-6.63 (m, 3H), 5.06 (dd, 1H), 4.97 (s, 2H), 4.31-4.20 (m, 2H), 3.96-3.74 (m, 6H), 3.54-3.35 (m, 4H), 3.35-3.22 (m, 1H), 3.10-2.80 (m, 3H), 2.71-2.50 (m, 2H), 2.20-1.82 (m, 5H), 1.64 (s, 6H).

LCMS m/z=813.3 [M+1]⁺.

### Example 71

### 3-chloro-5-(2-(4-((2-(4-(1-(1-((1-(2,6-dioxopiperidin-3-yl)-3-methyl-2-oxo-2,3-dihydro-1H-benzo[d]imidazol-5-yl)methyl)azetidin-3-yl)piperidin-4-yl)piperazin-1-yl)pyrimidin-4-yl)methoxy)phenyl)propan-2-yl)benzonitrile (Compound 71) trifluoroacetate

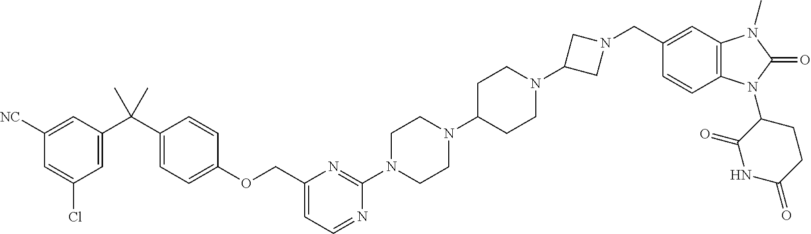 [SMILES: Cn1c(=O)n(C2CCC(=O)NC2=O)c2ccc(CN3CC(N4CCC(N5CCN(c6nccc(COc7ccc(C(C)(C)c8cc(Cl)cc(C#N)c8)cc7)n6)CC5)CC4)C3)cc21]

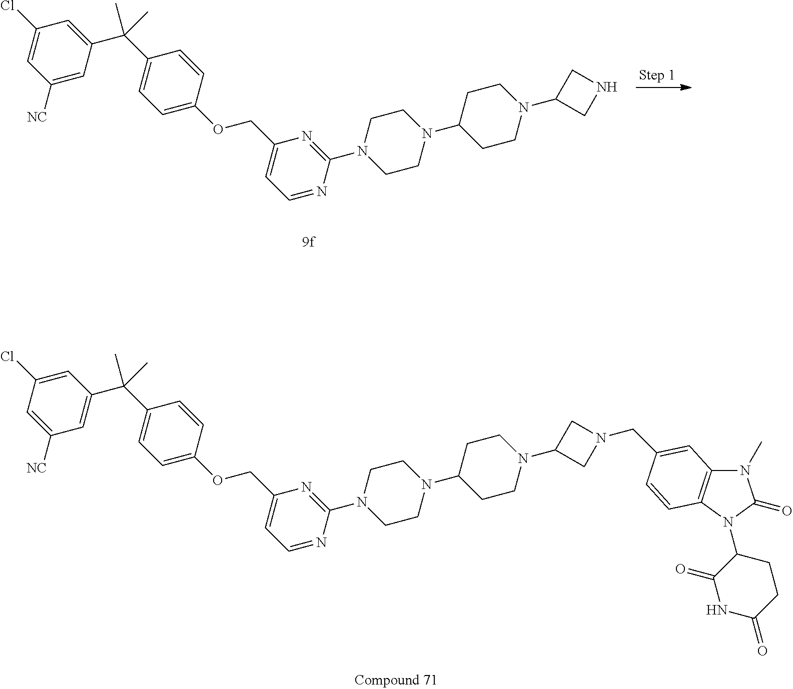 [SMILES: CC(C)(c1ccc(OCc2ccnc(N3CCN(C4CCN(C5CNC5)CC4)CC3)n2)cc1)c1cc(Cl)cc(C#N)c1 ; Cn1c(=O)n(C2CCC(=O)NC2=O)c2ccc(CN3CC(N4CCC(N5CCN(c6nccc(COc7ccc(C(C)(C)c8cc(Cl)cc(C#N)c8)cc7)n6)CC5)CC4)C3)cc21]

The above crude 3-(2-(4-((2-(4-(1-(azetidin-3-yl)piperidin-4-yl)piperazin-1-yl)pyrimidin-4-yl)methoxy)phenyl)propan-2-yl)-5-chlorobenzonitrile (9f) (70 mg) was dissolved in 4 mL of anhydrous tetrahydrofuran, and solid sodium bicarbonate (25 mg, 0.30 mmol) was added. The mixture was stirred at room temperature for 10 min, and then 1-(2,6-dioxopiperidin-3-yl)-3-methyl-2-oxo-2,3-dihydro-1H-benzo[d]imidazole-5-carbaldehyde (see WO 2020113233 for the synthetic method) (42 mg, 0.15 mmol) and 0.02 mL of acetic acid were successively added. The mixture was stirred at room temperature for 2 h, and then sodium triacetoxyborohydride (31 mg, 0.15 mmol) was added. The resulting mixture was reacted at room temperature for 16 h. To the reaction liquid was added solid sodium bicarbonate (100 mg), and the mixture was stirred at room temperature for 15 min and then filtered. The filtrate was concentrated under reduced pressure, and the crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 71 trifluoroacetate (12 mg).

¹H NMR (400 MHz, DMSO-d₆) δ 11.11 (s, 1H), 8.46 (d, 1H), 7.87-7.82 (m, 1H), 7.68-7.62 (m, 1H), 7.55-7.50 (m, 1H), 7.34-7.29 (m, 1H), 7.26-7.12 (m, 4H), 7.00-6.91 (m, 2H), 6.85 (d, 1H), 5.40 (dd, 1H), 5.02 (s, 2H), 4.95-4.47 (m, 2H), 4.45-4.30 (m, 2H), 4.20-4.01 (m, 4H), 3.89-3.81 (m, 1H), 3.54-3.40 (m, 4H), 3.36 (s, 3H), 3.34-2.97 (m, 7H), 2.97-2.84 (m, 1H), 2.81-2.57 (m, 2H), 2.22-1.96 (m, 3H), 1.80-1.57 (m, 8H).

LCMS m/z=857.4 [M+1]⁺

### Example 72

### 3-chloro-5-(2-(4-((2-(5-((1-(2-(2,6-dioxopiperidin-3-yl)-1,3-dioxoisoindolin-5-yl)piperidin-4-yl)methyl)hexahydropyrrolo[3,4-c]pyrrol-2(1H)-yl)pyrimidin-4-yl)methoxy)phenyl) propan-2-yl)benzonitrile (Compound 72) trifluoroacetate

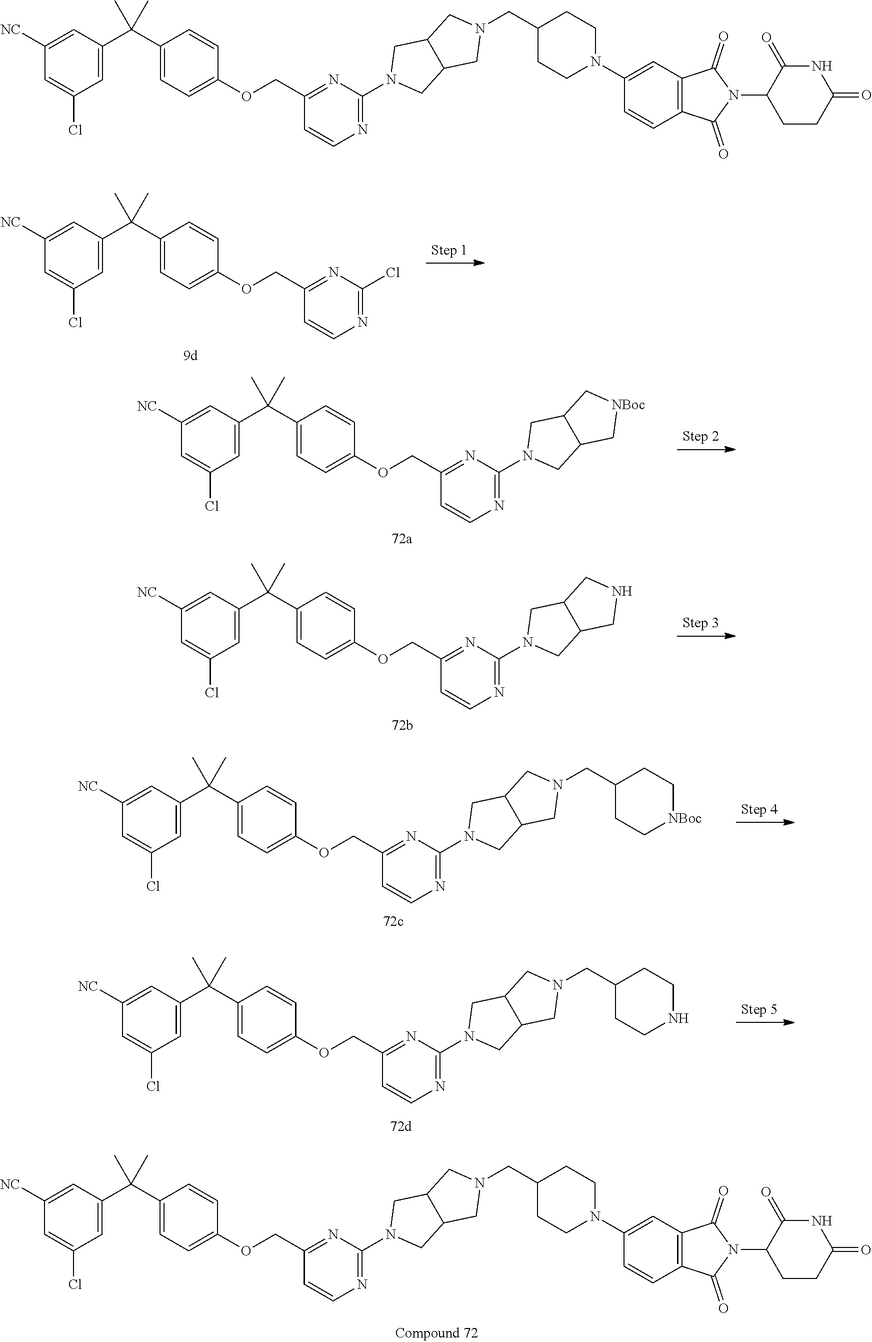

### Step 1: tert-butyl-5-(4-((4-(2-(3-chloro-5-cyanophenyl)propan-2-yl)phenoxy)methyl)pyrimidin-2-yl)hexahydropyrrolo[3,4-c]pyrrole-2(1H)-carboxylate (72a)

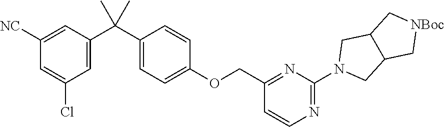 [SMILES: CC(C)(C)OC(=O)N1CC2CN(c3nccc(COc4ccc(C(C)(C)c5cc(Cl)cc(C#N)c5)cc4)n3)CC2C1]

3-chloro-5-(2-(4-((2-chloropyrimidin-4-yl)methoxy)phenyl)propan-2-yl)benzonitrile (9d) (0.25 g, 0.63 mmol) was dissolved in 5 mL of N,N-dimethylformamide, and tert-butyl hexahydropyrrolo[3,4-c]pyrrole-2(1H)-carboxylate (0.20 g, 0.94 mmol) and solid potassium bicarbonate (0.16 g, 1.60 mmol) were successively added. The mixture was warmed to 60° C. and reacted for 2 h. The reaction liquid was cooled to room temperature, and 30 mL of water was added. The mixture was extracted with ethyl acetate (30 mL×3), and the organic phase was washed with 100 mL of saturated sodium chloride solution, dried over anhydrous sodium sulfate, and concentrated under reduced pressure. The crude product was separated and purified by silica gel column chromatography (dichloromethane/methanol (v/v)=99:1), to afford 72a (0.25 g, yield: 69%).

### Step 2: 3-chloro-5-(2-(4-((2-(hexahydropyrrolo[3,4-c]pyrrol-2(1H)-yl)pyrimidin-4-yl)methoxy)phenyl)propan-2-yl)benzonitrile (72b) trifluoroacetate

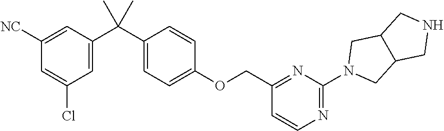 [SMILES: CC(C)(c1ccc(OCc2ccnc(N3CC4CNCC4C3)n2)cc1)c1cc(Cl)cc(C#N)c1]

72a (0.25 g, 0.44 mmol) was dissolved in 2 mL of dichloromethane, and trifluoroacetic acid (3.06 g, 26.84 mmol) was added. The mixture was stirred at room temperature for 2 h. The reaction system was concentrated under reduced pressure, to afford crude 72b trifluoroacetate (0.26 g).

### Step 3: tert-butyl 4-((5-(4-((4-(2-(3-chloro-5-cyanophenyl)propan-2-yl)phenoxy)methyl)pyrimidin-2-yl)hexahydropyrrolo[3,4-c]pyrrol-2(1H)-yl)methyl)piperidine-1-carboxylate (72c)

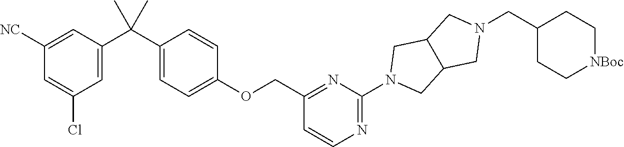 [SMILES: CC(C)(C)OC(=O)N1CCC(CN2CC3CN(c4nccc(COc5ccc(C(C)(C)c6cc(Cl)cc(C#N)c6)cc5)n4)CC3C2)CC1]

The above crude 72b trifluoroacetate (0.13 g) was dissolved in 5 mL of dichloromethane, and solid sodium bicarbonate (0.11 g, 1.31 mmol) was added. The mixture was stirred at room temperature for 0.5 h, and then tert-butyl 4-formylpiperidine-1-carboxylate (0.046 g, 0.22 mmol) and sodium triacetoxyborohydride (0.11 g, 0.52 mmol) were added, respectively. The resulting mixture was reacted at room temperature for 16 h. To the reaction system was added 5 mL of saturated sodium bicarbonate solution. The mixture was extracted with ethyl acetate (10 mL×3), and the organic phase was washed with 20 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 72c (0.14 g, two-step yield from compound 72a: 95%).

LCMS m/z=671.3 [M+1]⁺

### Step 4: 3-chloro-5-(2-(4-((2-(5-(piperidin-4-ylmethyl)hexahydropyrrolo[3,4-c]pyrrol-2(1H)-yl)pyrimidin-4-yl)methoxy)phenyl)propan-2-yl)benzonitrile (72d) trifluoroacetate

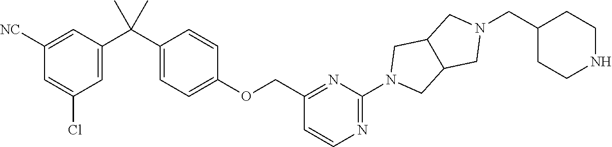 [SMILES: CC(C)(c1ccc(OCc2ccnc(N3CC4CN(CC5CCNCC5)CC4C3)n2)cc1)c1cc(Cl)cc(C#N)c1]

72c (0.14 g, 0.21 mmol) was dissolved in 2 mL of dichloromethane, and trifluoroacetic acid (3.06 g, 26.84 mmol) was added. The mixture was reacted at room temperature for 2 h. The reaction system was concentrated under reduced pressure, to afford crude 72d trifluoroacetate (0.15 g).

### Step 5: 3-chloro-5-(2-(4-((2-(5-((1-(2-(2,6-dioxopiperidin-3-yl)-1,3-dioxoisoindolin-5-yl)piperidin-4-yl)methyl)hexahydropyrrolo[3,4-c]pyrrol-2(1H)-yl)pyrimidin-4-yl)methoxy)phenyl)propan-2-yl)benzonitrile (Compound 72) trifluoroacetate

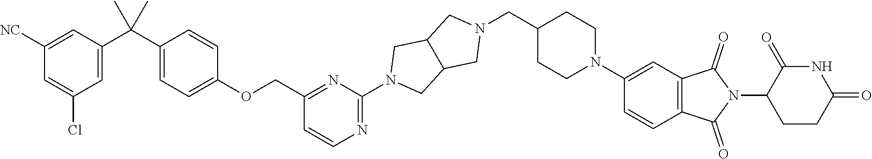 [SMILES: CC(C)(c1ccc(OCc2ccnc(N3CC4CN(CC5CCN(c6ccc7c(c6)C(=O)N(C6CCC(=O)NC6=O)C7=O)CC5)CC4C3)n2)cc1)c1cc(Cl)cc(C#N)c1]

To a reaction flask were respectively added the above crude 72d trifluoroacetate (0.15 g), 2-(2,6-dioxopiperidin-3-yl)-5-fluoroisoindoline-1,3-dione (see WO 2017197056 for the synthetic method) (0.20 g, 0.72 mmol) and diisopropylethylamine (0.3 mL). 5 mL of DMSO was added, and the mixture was warmed to 80° C. and reacted for 4 h. The reaction liquid was cooled to room temperature, and 20 mL of water was added. The mixture was extracted with dichloromethane (20 mL×3), and the organic phase was washed with 20 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 72 trifluoroacetate (35 mg).

¹H NMR (400 MHz, DMSO-d₆) δ 11.06 (s, 1H), 8.39 (d, 1H), 7.87-7.80 (m, 1H), 7.71-7.60 (m, 2H), 7.55-7.50 (m, 1H), 7.39-7.22 (m, 2H), 7.21-7.13 (m, 2H), 7.00-6.90 (m, 2H), 6.77 (d, 1H), 5.12-4.92 (m, 3H), 4.15-3.86 (m, 3H), 3.85-3.55 (m, 4H), 3.45-2.75 (m, 10H), 2.70-2.50 (m, 2H), 2.13-1.93 (m, 2H), 1.92-1.77 (m, 2H), 1.64 (s, 6H), 1.37-1.15 (m, 2H).

LCMS m/z=827.3 [M+1]⁺.

### Example 73

### 3-chloro-5-(2-(4-((2-(5-((1-(2-(2,6-dioxopiperidin-3-yl)-1,3-dioxoisoindolin-5-yl)azetidin-3-yl)methyl)hexahydropyrrolo[3,4-c]pyrrol-2(1H)-yl)pyrimidin-4-yl)methoxy)phenyl) propan-2-yl)benzonitrile (Compound 73) trifluoroacetate

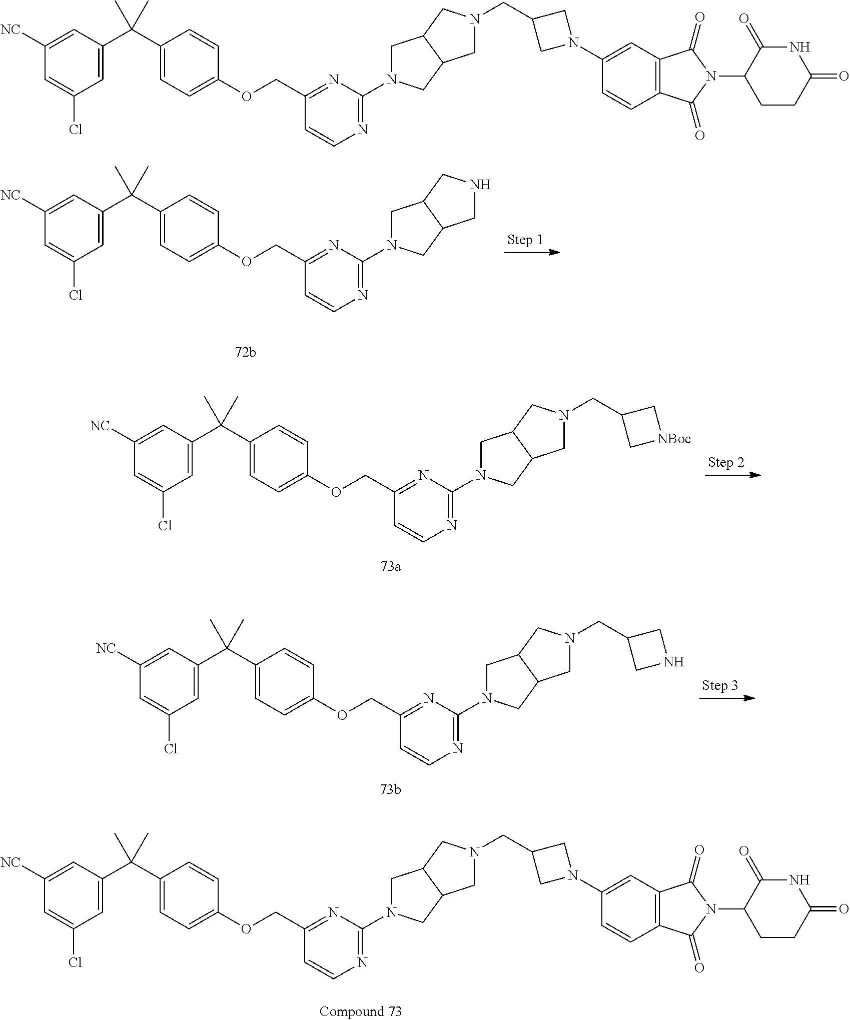 [SMILES: CC(C)(c1ccc(OCc2ccnc(N3CC4CN(CC5CN(c6ccc7c(c6)C(=O)N(C6CCC(=O)NC6=O)C7=O)C5)CC4C3)n2)cc1)c1cc(Cl)cc(C#N)c1 ; CC(C)(c1ccc(OCc2ccnc(N3CC4CNCC4C3)n2)cc1)c1cc(Cl)cc(C#N)c1 ; CC(C)(C)OC(=O)N1CC(CN2CC3CN(c4nccc(COc5ccc(C(C)(C)c6cc(Cl)cc(C#N)c6)cc5)n4)CC3C2)C1 ; CC(C)(c1ccc(OCc2ccnc(N3CC4CN(CC5CNC5)CC4C3)n2)cc1)c1cc(Cl)cc(C#N)c1 ; CC(C)(c1ccc(OCc2ccnc(N3CC4CN(CC5CN(c6ccc7c(c6)C(=O)N(C6CCC(=O)NC6=O)C7=O)C5)CC4C3)n2)cc1)c1cc(Cl)cc(C#N)c1]

### Step 1: tert-butyl 3-((5-(4-((4-(2-(3-chloro-5-cyanophenyl)propan-2-yl)phenoxy)methyl)pyrimidin-2-yl)hexahydropyrrolo[3,4-c]pyrrol-2(1H)-yl)methyl)azetidine-1-carboxylate (73a)

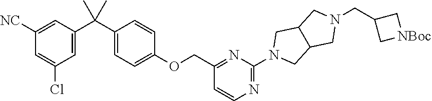 [SMILES: CC(C)(C)OC(=O)N1CC(CN2CC3CN(c4nccc(COc5ccc(C(C)(C)c6cc(Cl)cc(C#N)c6)cc5)n4)CC3C2)C1]

The above crude 72b trifluoroacetate (0.13 g) was dissolved in 5 mL of dichloromethane, and solid sodium bicarbonate (0.11 g, 1.31 mmol) was added. The mixture was stirred at room temperature for 0.5 h, and then tert-butyl 3-formylazetidine-1-carboxylate (0.046 g, 0.25 mmol) and sodium triacetoxyborohydride (0.11 g, 0.52 mmol) were added, respectively. The resulting mixture was reacted at room temperature for 16 h. To the reaction system was added 5 mL of saturated sodium bicarbonate solution. The mixture was extracted with ethyl acetate (10 mL×3), and the organic phase was washed with 20 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 73a (0.16 g, two-step yield from compound 72a: >99%).

LCMS m/z=643.3 [M+1]⁺

### Step 2: 3-(2-(4-((2-(5-(azetidin-3-ylmethyl)hexahydropyrrolo[3,4-c]pyrrol-2(1H)-yl)pyrimidin-4-yl)methoxy)phenyl)propan-2-yl)-5-chlorobenzonitrile (73b) trifluoroacetate

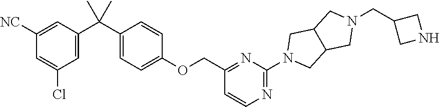 [SMILES: CC(C)(c1ccc(OCc2ccnc(N3CC4CN(CC5CNC5)CC4C3)n2)cc1)c1cc(Cl)cc(C#N)c1]

73a (0.16 g, 0.25 mmol) was dissolved in 2 mL of dichloromethane, and trifluoroacetic acid (3.06 g, 26.84 mmol) was added. The mixture was stirred at room temperature for 2 h. The reaction system was concentrated under reduced pressure, to afford crude 73b trifluoroacetate (0.18 g).

### Step 3: 3-chloro-5-(2-(4-((2-(5-((1-(2-(2,6-dioxopiperidin-3-yl)-1,3-dioxoisoindolin-5-yl)azetidin-3-yl)methyl)hexahydropyrrolo[3,4-c]pyrrol-2(1H)-yl)pyrimidin-4-yl)methoxy)phenyl)propan-2-yl)benzonitrile (Compound 73) trifluoroacetate

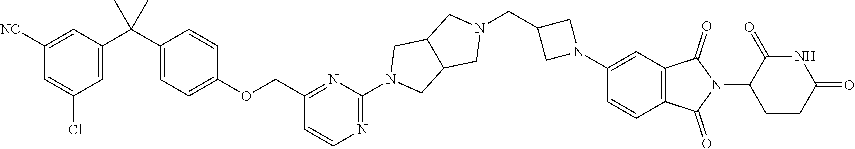 [SMILES: CC(C)(c1ccc(OCc2ccnc(N3CC4CN(CC5CN(c6ccc7c(c6)C(=O)N(C6CCC(=O)NC6=O)C7=O)C5)CC4C3)n2)cc1)c1cc(Cl)cc(C#N)c1]

To a reaction flask were respectively added the above crude 73b trifluoroacetate (0.18 g), 2-(2,6-dioxopiperidin-3-yl)-5-fluoroisoindoline-1,3-dione (see WO 2017197056 for the synthetic method) (0.20 g, 0.72 mmol) and diisopropylethylamine (0.3 mL). 5 mL of DMSO was added, and the mixture was warmed to 80° C. and reacted for 4 h. The reaction liquid was cooled to room temperature, and 20 mL of water was added. The mixture was extracted with dichloromethane (20 mL×3), and the organic phase was washed with 20 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 73 trifluoroacetate (45 mg).

¹H NMR (400 MHz, DMSO-d₆) δ 11.05 (s, 1H), 8.43-8.36 (m, 1H), 7.87-7.82 (m, 1H), 7.71-7.62 (m, 2H), 7.56-7.49 (m, 1H), 7.22-7.13 (m, 2H), 7.00-6.90 (m, 2H), 6.83-6.74 (m, 2H), 6.71-6.62 (m, 1H), 5.10-4.94 (m, 3H), 4.27-4.15 (m, 2H), 3.94-3.68 (m, 5H), 3.65-3.47 (m, 5H), 3.45-3.04 (m, 4H), 3.03-2.78 (m, 2H), 2.64-2.45 (m, 2H), 2.06-1.95 (m, 1H), 1.64 (s, 6H).

LCMS m/z=799.3 [M+1]⁺.

### Example 74

### 3-chloro-5-(2-(4-((2-(1′-((1-((1-(2,6-dioxopiperidin-3-yl)-3-methyl-2-oxo-2,3-dihydro-1H-benzo[d]imidazol-4-yl)methyl)piperidin-4-yl)methyl)-[4,4′-bipiperidin]-1-yl)pyrimidin-4-yl)methoxy)phenyl)propan-2-yl)benzonitrile (Compound 74) trifluoroacetate

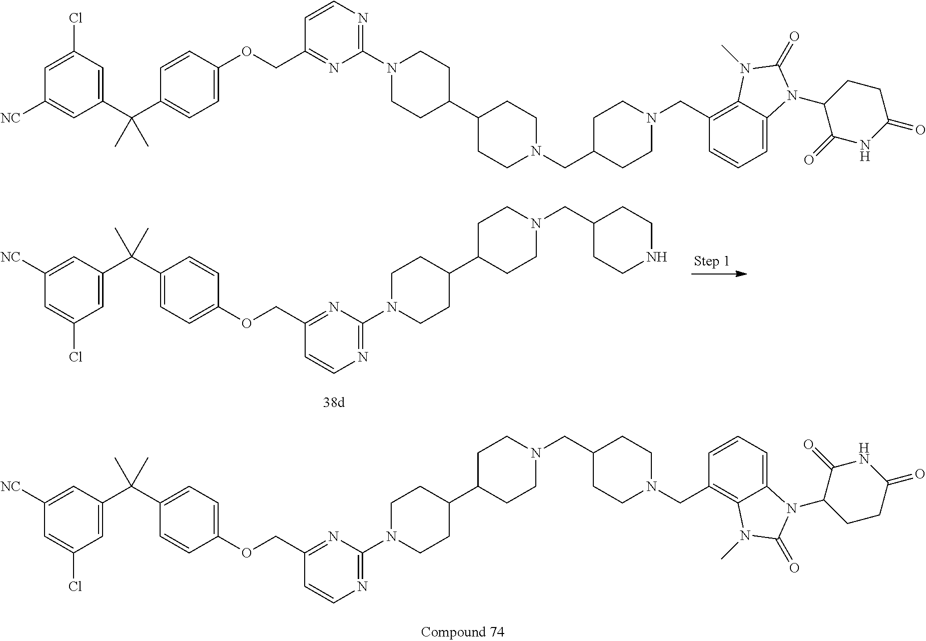

The above crude 38d trifluoroacetate (240 mg) was dissolved in 15 mL of THF, and solid sodium bicarbonate (91 mg, 1.08 mmol) was added. The mixture was stirred at room temperature for 20 min, and then 1-(2,6-dioxopiperidin-3-yl)-3-methyl-2-oxo-2,3-dihydro-1H-benzo[d]imidazole-4-carbaldehyde (see WO 2020113233 for the synthetic method) (118 mg, 0.41 mmol) and 0.05 mL of acetic acid were successively added. The mixture was stirred at room temperature for 60 min, and then sodium triacetoxyborohydride (115 mg, 0.54 mmol) was added. The resulting mixture was reacted at room temperature for 16 h. To the reaction system was slowly added 20 mL of saturated sodium bicarbonate solution, and the mixture was extracted twice with 80 mL of DCM. The organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 74 trifluoroacetate (8 mg).

¹H NMR (400 MHz, DMSO-d₆) δ 11.12 (s, 1H), 8.33 (d, 1H), 7.88-7.80 (m, 1H), 7.68-7.62 (m, 1H), 7.56-7.50 (m, 1H), 7.35-7.05 (m, 5H), 6.98-6.90 (m, 2H), 6.66 (d, 1H), 5.44 (dd, 1H), 4.96 (s, 2H), 4.78-4.52 (m, 4H), 3.61 (s, 3H), 3.59-3.42 (m, 4H), 3.38-3.02 (m, 4H), 3.02-2.57 (m, 8H), 2.20-1.55 (m, 14H), 1.55-1.28 (m, 5H), 1.20-1.01 (m, 2H).

LCMS m/z=898.4 [M+1]⁺

### Example 75

### 3-chloro-5-(2-(4-((2-(2-(1-(1-(2-(2,6-dioxopiperidin-3-yl)-1,3-dioxoisoindolin-5-yl)azetidin-3-yl)piperidin-4-yl)-2,7-diazaspiro[3.5]nonan-7-yl)pyrimidin-4-yl)methoxy)phenyl)propan-2-yl)benzonitrile (Compound 75) trifluoroacetate

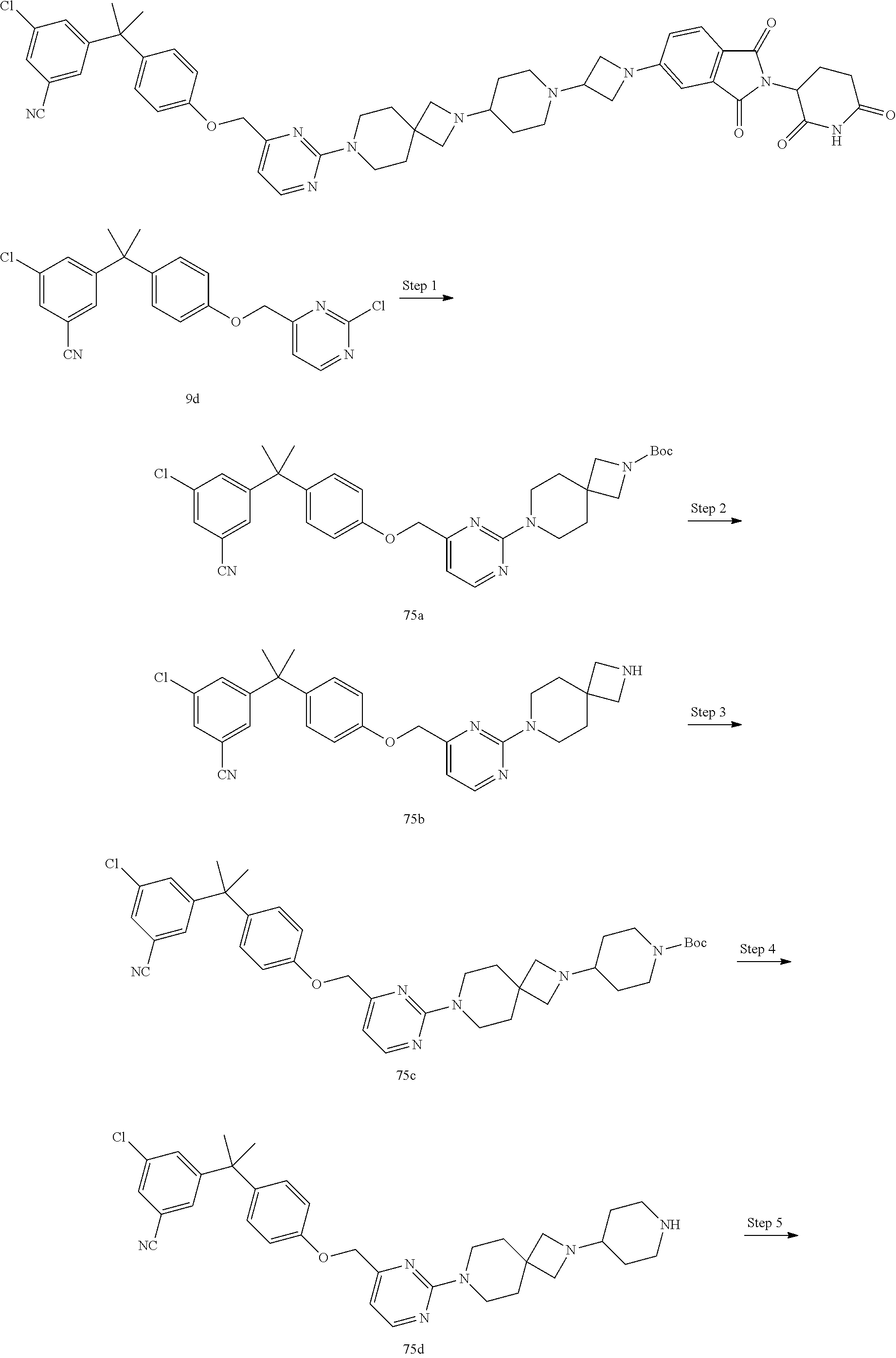 [SMILES: CC(C)(c1ccc(OCc2ccnc(N3CCC4(CC3)CN(C3CCN(C5CN(c6ccc7c(c6)C(=O)N(C6CCC(=O)NC6=O)C7=O)C5)CC3)C4)n2)cc1)c1cc(Cl)cc(C#N)c1 ; CC(C)(c1ccc(OCc2ccnc(Cl)n2)cc1)c1cc(Cl)cc(C#N)c1 ; CC(C)(C)OC(=O)N1CC2(CCN(c3nccc(COc4ccc(C(C)(C)c5cc(Cl)cc(C#N)c5)cc4)n3)CC2)C1 ; CC(C)(c1ccc(OCc2ccnc(N3CCC4(CC3)CNC4)n2)cc1)c1cc(Cl)cc(C#N)c1 ; CC(C)(C)OC(=O)N1CCC(N2CC3(CCN(c4nccc(COc5ccc(C(C)(C)c6cc(Cl)cc(C#N)c6)cc5)n4)CC3)C2)CC1 ; CC(C)(c1ccc(OCc2ccnc(N3CCC4(CC3)CN(C3CCNCC3)C4)n2)cc1)c1cc(Cl)cc(C#N)c1]

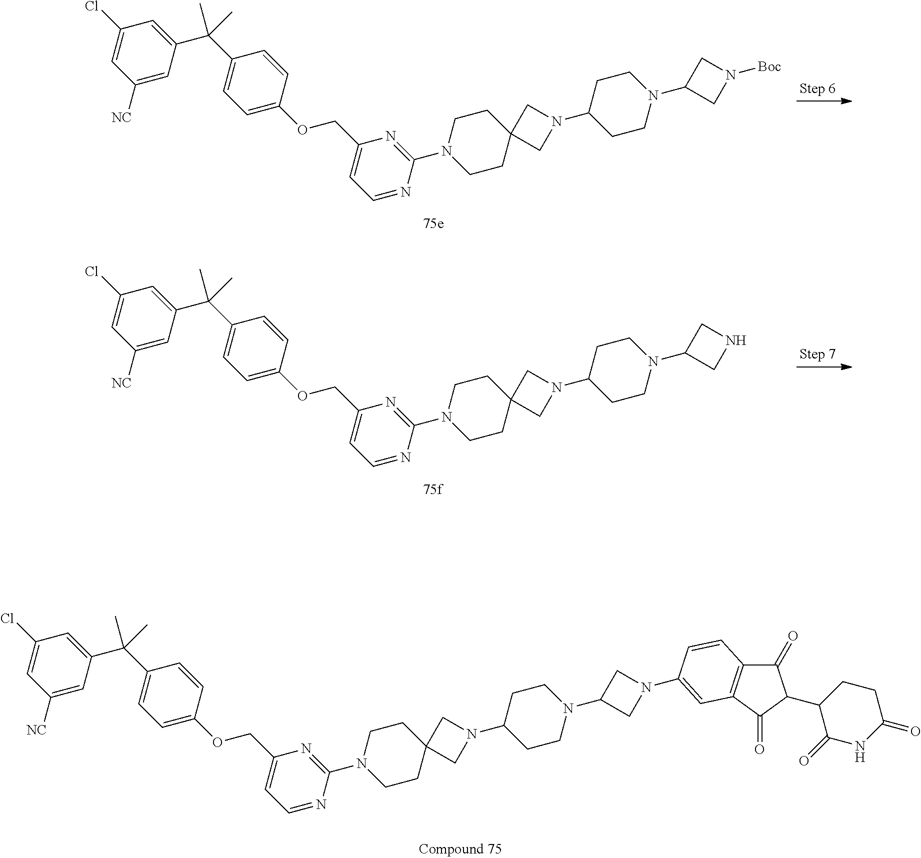 [SMILES: CC(C)(C)OC(=O)N1CC(N2CCC(N3CC4(CCN(c5nccc(COc6ccc(C(C)(C)c7cc(Cl)cc(C#N)c7)cc6)n5)CC4)C3)CC2)C1 ; CC(C)(c1ccc(OCc2ccnc(N3CCC4(CC3)CN(C3CCN(C5CNC5)CC3)C4)n2)cc1)c1cc(Cl)cc(C#N)c1 ; CC(C)(c1ccc(OCc2ccnc(N3CCC4(CC3)CN(C3CCN(C5CN(c6ccc7c(c6)C(=O)C(C6CCC(=O)NC6=O)C7=O)C5)CC3)C4)n2)cc1)c1cc(Cl)cc(C#N)c1]

### Step 1: tert-butyl 7-(4-((4-(2-(3-chloro-5-cyanophenyl)propan-2-yl)phenoxy)methyl)pyrimidin-2-yl)-2,7-diazaspiro[3.5]nonane-2-carboxylate (75a)

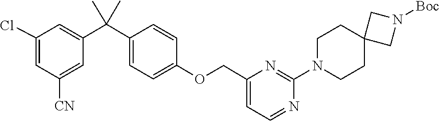 [SMILES: CC(C)(C)OC(=O)N1CC2(CCN(c3nccc(COc4ccc(C(C)(C)c5cc(Cl)cc(C#N)c5)cc4)n3)CC2)C1]

3-chloro-5-(2-(4-((2-chloropyrimidin-4-yl)methoxy)phenyl)propan-2-yl)benzonitrile (9d) (200 mg, 0.50 mmol) was dissolved in 15 ml of DMF, and tert-butyl 2,7-diazaspiro[3.5]nonane-2-carboxylate (170 mg, 0.75 mmol) and solid potassium bicarbonate (101 mg, 1.01 mmol) were added. The mixture was warmed to 60° C. and reacted for 3 h. The reaction liquid was cooled to room temperature, and 50 mL of water was added. The mixture was extracted with ethyl acetate (30 mL×3), and the organic phase was washed with water (50 mL×2), dried over anhydrous sodium sulfate, and concentrated under reduced pressure. The crude product was separated and purified by silica gel column chromatography (dichloromethane/methanol (v/v)=20:1), to afford 75a (280 mg, yield: 95%).

LCMS m/z=588.5 [M+1]⁺

### Step 2: 3-(2-(4-((2-(2,7-diazaspiro[3.5]nonan-7-yl)pyrimidin-4-yl)methoxy)phenyl)propan-2-yl)-5-chlorobenzonitrile (75b) trifluoroacetate

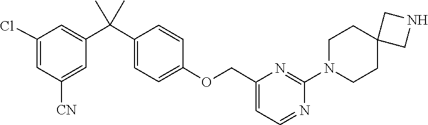 [SMILES: CC(C)(c1ccc(OCc2ccnc(N3CCC4(CC3)CNC4)n2)cc1)c1cc(Cl)cc(C#N)c1]

75a (120 mg, 0.20 mmol) was dissolved in 10 mL of DCM, and 4 mL of trifluoroacetic acid was added. The mixture was stirred at room temperature for 2 h. The reaction liquid was concentrated under reduced pressure, to afford crude 75b trifluoroacetate (135 mg).

LCMS m/z=488.2 [M+1]⁺

### Step 3: tert-butyl 4-(7-(4-((4-(2-(3-chloro-5-cyanophenyl)propan-2-yl)phenoxy)methyl)pyrimidin-2-yl)-2,7-diazaspiro[3.5]nonan-2-yl)piperidine-1-carboxylate (75c)

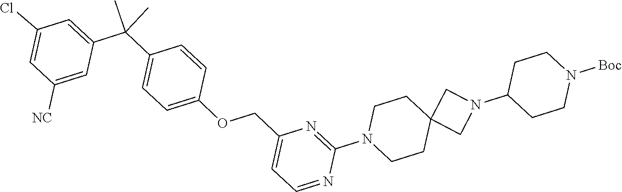 [SMILES: CC(C)(C)OC(=O)N1CCC(N2CC3(CCN(c4nccc(COc5ccc(C(C)(C)c6cc(Cl)cc(C#N)c6)cc5)n4)CC3)C2)CC1]

The above crude 75b trifluoroacetate (135 mg) was dissolved in 15 mL of THF, and solid sodium bicarbonate (50 mg, 0.60 mmol) was added. The mixture was stirred at room temperature for 20 min, and then tert-butyl 4-oxopiperidine-1-carboxylate (81 mg, 0.41 mmol) and 0.05 mL of acetic acid were successively added. The mixture was stirred at room temperature for 60 min, and then sodium triacetoxyborohydride (85 mg, 0.40 mmol) was added. The resulting mixture was reacted at room temperature for 16 h. To the reaction system was slowly added 20 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=15:1), to afford 75c (130 mg, two-step yield from compound 75a: 97%).

LCMS m/z=671.3 [M+1]⁺

### Step 4: 3-chloro-5-(2-(4-((2-(2-(piperidin-4-yl)-2,7-diazaspiro[3.5]nonan-7-yl)pyrimidin-4-yl)methoxy)phenyl)propan-2-yl)benzonitrile (75d) trifluoroacetate

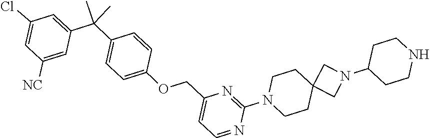 [SMILES: CC(C)(c1ccc(OCc2ccnc(N3CCC4(CC3)CN(C3CCNCC3)C4)n2)cc1)c1cc(Cl)cc(C#N)c1]

75c (70 mg, 0.10 mmol) was dissolved in 10 mL of DCM, and 4 mL of trifluoroacetic acid was added. The mixture was reacted at room temperature for 2 h. The reaction liquid was concentrated under reduced pressure, to afford crude 75d trifluoroacetate (75 mg).

LCMS m/z=571.3 [M+1]⁺

### Step 5: tert-butyl 3-(4-(7-(4-((4-(2-(3-chloro-5-cyanophenyl)propan-2-yl)phenoxy)methyl)pyrimidin-2-yl)-2,7-diazaspiro[3.5]nonan-2-yl)piperidin-1-yl)azetidine-1-carboxylate (75e)

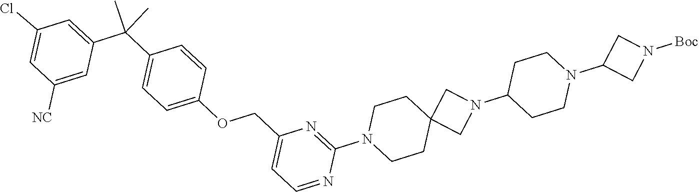 [SMILES: CC(C)(C)OC(=O)N1CC(N2CCC(N3CC4(CCN(c5nccc(COc6ccc(C(C)(C)c7cc(Cl)cc(C#N)c7)cc6)n5)CC4)C3)CC2)C1]

The above crude 75d trifluoroacetate (75 mg) was dissolved in 15 mL of THF, and solid sodium bicarbonate (36 mg, 0.43 mmol) was added. The mixture was stirred at room temperature for 20 min, and then tert-butyl 3-oxoazetidine-1-carboxylate (36 mg, 0.21 mmol) and 0.05 mL of acetic acid were successively added. The mixture was stirred at room temperature for 60 min, and then sodium triacetoxyborohydride (45 mg, 0.21 mmol) was added. The resulting mixture was reacted at room temperature for 16 h. To the reaction system was slowly added 20 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=8:1), to afford 75e (45 mg, two-step yield from compound 75c: 62%).

LCMS m/z=726.4 [M+1]⁺

### Step 6: 3-(2-(4-((2-(2-(1-(azetidin-3-yl)piperidin-4-yl)-2,7-diazaspiro[3.5]nonan-7-yl)pyrimidin-4-yl)methoxy)phenyl)propan-2-yl)-5-chlorobenzonitrile (75f) trifluoroacetate

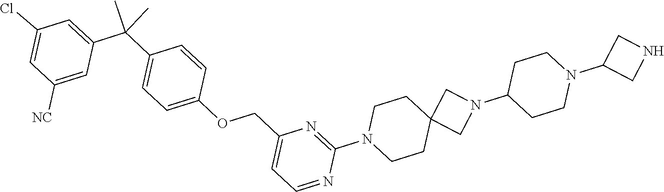 [SMILES: CC(C)(c1ccc(OCc2ccnc(N3CCC4(CC3)CN(C3CCN(C5CNC5)CC3)C4)n2)cc1)c1cc(Cl)cc(C#N)c1]

75e (45 mg, 0.062 mmol) was dissolved in 5 mL of DCM, and 2 mL of trifluoroacetic acid was added. The mixture was reacted at room temperature for 2 h. The reaction liquid was concentrated under reduced pressure, to afford crude 75f trifluoroacetate (60 mg).

LCMS m/z=626.3 [M+1]⁺

### Step 7: 3-chloro-5-(2-(4-((2-(2-(1-(1-(2-(2,6-dioxopiperidin-3-yl)-1,3-dioxoisoindolin-5-yl)azetidin-3-yl)piperidin-4-yl)-2,7-diazaspiro[3.5]nonan-7-yl)pyrimidin-4-yl)methoxy)phenyl)propan-2-yl)benzonitrile (Compound 75) trifluoroacetate

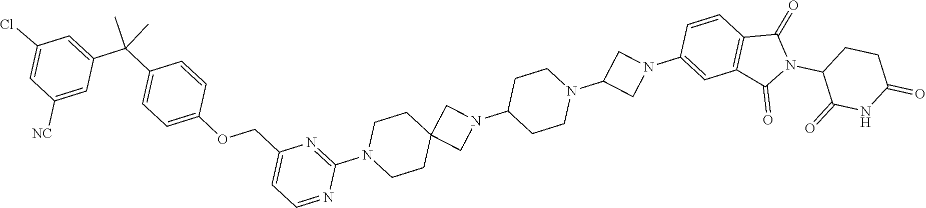 [SMILES: CC(C)(c1ccc(OCc2ccnc(N3CCC4(CC3)CN(C3CCN(C5CN(c6ccc7c(c6)C(=O)N(C6CCC(=O)NC6=O)C7=O)C5)CC3)C4)n2)cc1)c1cc(Cl)cc(C#N)c1]

The above crude 75f trifluoroacetate (60 mg) was dissolved in 4 mL of DMSO, and solid sodium bicarbonate (26 mg, 0.31 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (25 mg, 0.091 mmol) were added. The resulting mixture was warmed to 80° C. and reacted for 5 h. The reaction liquid was cooled to room temperature, and 50 mL of water was added. The mixture was filtered, and the solid (filter cake) was collected, washed with 20 mL of water, dissolved with 100 mL of DCM, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 75 trifluoroacetate (15 mg).

¹H NMR (400 MHz, DMSO-d₆) δ 11.06 (s, 1H), 8.36 (d, 1H), 7.87-7.82 (m, 1H), 7.72 (d, 1H), 7.66-7.63 (m, 1H), 7.54-7.51 (m, 1H), 7.21-7.13 (m, 2H), 6.98-6.90 (m, 2H), 6.90-6.84 (m, 1H), 6.78-6.66 (m, 2H), 5.07 (dd, 1H), 4.97 (s, 2H), 4.38-3.92 (m, 10H), 3.84-3.35 (m, 7H), 2.97-2.81 (m, 1H), 2.80-2.51 (m, 3H), 2.25-1.95 (m, 3H), 1.94-1.70 (m, 4H), 1.70-1.40 (m, 8H).

LCMS m/z=882.3 [M+1]⁺.

### Example 76

### 3-chloro-5-(2-(4-((2-(2-(1′-(2-(2,6-dioxopiperidin-3-yl)-1,3-dioxoisoindolin-5-yl)-[1,3′-biazetidin]-3-yl)-2,7-diazaspiro[3.5]nonan-7-yl)pyrimidin-4-yl)methoxy)phenyl)propan-2-yl)benzonitrile (Compound 76) trifluoroacetate

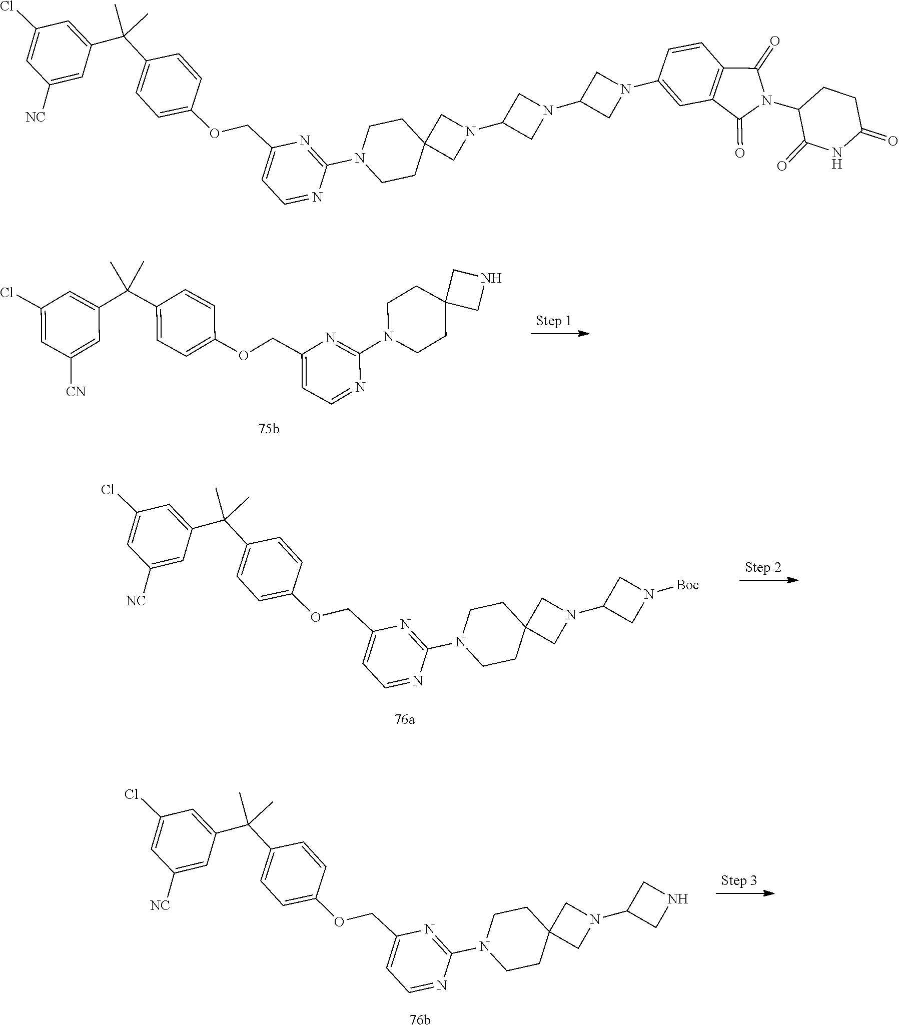 [SMILES: CC(C)(c1ccc(OCc2ccnc(N3CCC4(CC3)CN(C3CN(C5CN(c6ccc7c(c6)C(=O)N(C6CCC(=O)NC6=O)C7=O)C5)C3)C4)n2)cc1)c1cc(Cl)cc(C#N)c1 ; CC(C)(c1ccc(OCc2ccnc(N3CCC4(CC3)CNC4)n2)cc1)c1cc(Cl)cc(C#N)c1 ; CC(C)(C)OC(=O)N1CC(N2CC3(CCN(c4nccc(COc5ccc(C(C)(C)c6cc(Cl)cc(C#N)c6)cc5)n4)CC3)C2)C1 ; CC(C)(c1ccc(OCc2ccnc(N3CCC4(CC3)CN(C3CNC3)C4)n2)cc1)c1cc(Cl)cc(C#N)c1]

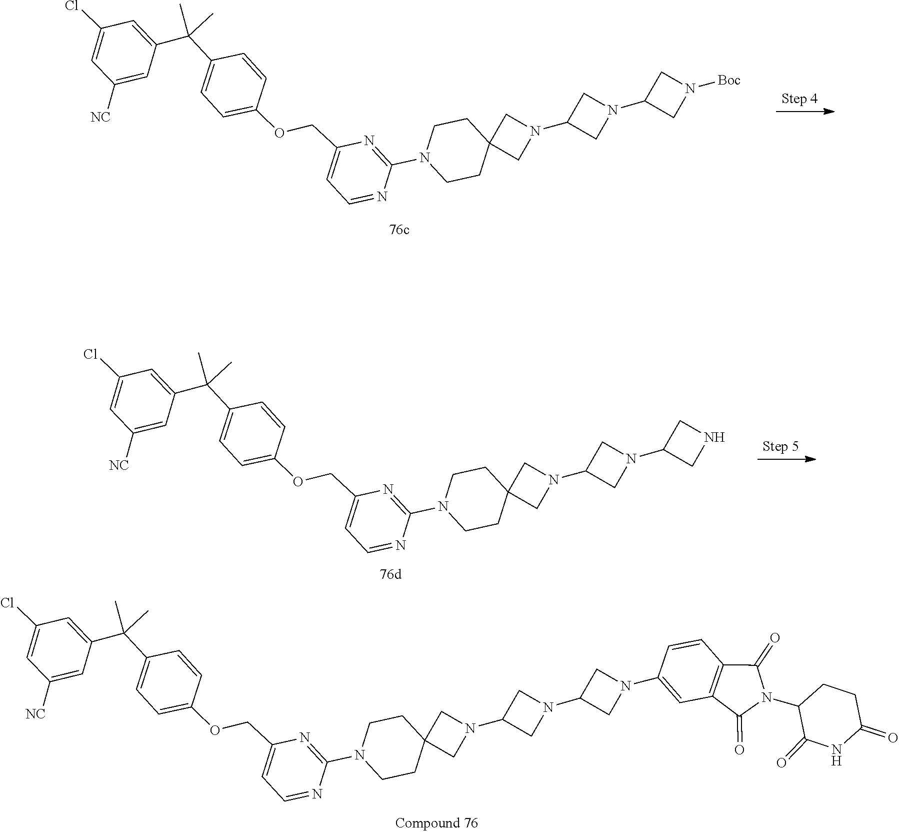 [SMILES: CC(C)(C)OC(=O)N1CC(N2CC(N3CC4(CCN(c5nccc(COc6ccc(C(C)(C)c7cc(Cl)cc(C#N)c7)cc6)n5)CC4)C3)C2)C1 ; CC(C)(c1ccc(OCc2ccnc(N3CCC4(CC3)CN(C3CN(C5CNC5)C3)C4)n2)cc1)c1cc(Cl)cc(C#N)c1 ; CC(C)(c1ccc(OCc2ccnc(N3CCC4(CC3)CN(C3CN(C5CN(c6ccc7c(c6)C(=O)N(C6CCC(=O)NC6=O)C7=O)C5)C3)C4)n2)cc1)c1cc(Cl)cc(C#N)c1]

### Step 1: tert-butyl 3-(7-(4-((4-(2-(3-chloro-5-cyanophenyl)propan-2-yl)phenoxy)methyl)pyrimidin-2-yl)-2,7-diazaspiro[3.5]nonan-2-yl)azetidine-1-carboxylate (76a)

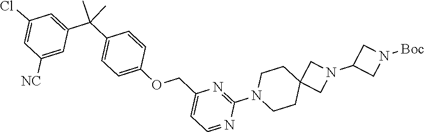 [SMILES: CC(C)(C)OC(=O)N1CC(N2CC3(CCN(c4nccc(COc5ccc(C(C)(C)c6cc(Cl)cc(C#N)c6)cc5)n4)CC3)C2)C1]

The above crude 75b trifluoroacetate (135 mg) was dissolved in 15 mL of THF, and solid sodium bicarbonate (50 mg, 0.60 mmol) was added. The mixture was stirred at room temperature for 20 min, and then tert-butyl 3-oxoazetidine-1-carboxylate (70 mg, 0.41 mmol) and 0.05 mL of acetic acid were successively added. The mixture was stirred at room temperature for 60 min, and then sodium triacetoxyborohydride (85 mg, 0.40 mmol) was added. The resulting mixture was reacted at room temperature for 16 h. To the reaction system was slowly added 20 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=15:1), to afford 76a (120 mg, two-step yield from compound 75a: >99%).

LCMS m/z=643.3 [M+1]⁺

### Step 2: 3-(2-(4-((2-(2-(azetidin-3-yl)-2,7-diazaspiro[3.5]nonan-7-yl)pyrimidin-4-yl)methoxy)phenyl)propan-2-yl)-5-chlorobenzonitrile (76b) trifluoroacetate

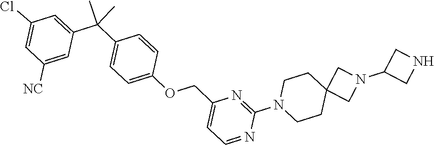 [SMILES: CC(C)(c1ccc(OCc2ccnc(N3CCC4(CC3)CN(C3CNC3)C4)n2)cc1)c1cc(Cl)cc(C#N)c1]

76a (60 mg, 0.093 mmol) was dissolved in 10 mL of DCM, and 4 mL of trifluoroacetic acid was added. The mixture was reacted at room temperature for 2 h. The reaction liquid was concentrated under reduced pressure, to afford crude 76b trifluoroacetate (70 mg).

LCMS m/z=543.3 [M+1]⁺

### Step 3: tert-butyl 3-(7-(4-((4-(2-(3-chloro-5-cyanophenyl)propan-2-yl)phenoxy)methyl)pyrimidin-2-yl)-2,7-diazaspiro[3.5]nonan-2-yl)-[1,3′-biazetidine]-1′-carboxylate (76c)

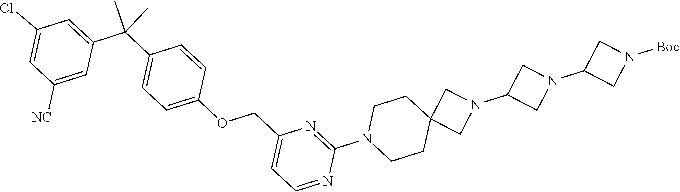 [SMILES: CC(C)(C)OC(=O)N1CC(N2CC(N3CC4(CCN(c5nccc(COc6ccc(C(C)(C)c7cc(Cl)cc(C#N)c7)cc6)n5)CC4)C3)C2)C1]

The above crude 76b trifluoroacetate (70 mg) was dissolved in 15 mL of THF, and solid sodium bicarbonate (32 mg, 0.38 mmol) was added. The mixture was stirred at room temperature for 20 min, and then tert-butyl 3-oxoazetidine-1-carboxylate (32 mg, 0.19 mmol) and 0.05 mL of acetic acid were successively added. The mixture was stirred at room temperature for 60 min, and then sodium triacetoxyborohydride (40 mg, 0.19 mmol) was added. The resulting mixture was reacted at room temperature for 16 h. To the reaction system was slowly added 20 mL of saturated sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=8:1), to afford 76c (45 mg, two-step yield from compound 76a: 69%).

LCMS m/z=698.3 [M+1]⁺

### Step 4: 3-(2-(4-((2-(2-([1,3′-biazetidin]-3-yl)-2,7-diazaspiro[3.5]nonan-7-yl)pyrimidin-4-yl)methoxy)phenyl)propan-2-yl)-5-chlorobenzonitrile (76d) trifluoroacetate

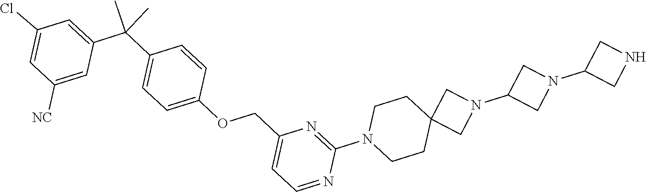 [SMILES: CC(C)(c1ccc(OCc2ccnc(N3CCC4(CC3)CN(C3CN(C5CNC5)C3)C4)n2)cc1)c1cc(Cl)cc(C#N)c1]

76c (45 mg, 0.065 mmol) was dissolved in 5 mL of DCM, and 2 mL of trifluoroacetic acid was added. The mixture was reacted at room temperature for 2 h. The reaction liquid was concentrated under reduced pressure, to afford crude 76d trifluoroacetate (60 mg).

LCMS m/z=598.3 [M+1]⁺

### Step 5: 3-chloro-5-(2-(4-((2-(2-(1′-(2-(2,6-dioxopiperidin-3-yl)-1,3-dioxoisoindolin-5-yl)-[1,3′-biazetidin]-3-yl)-2,7-diazaspiro[3.5]nonan-7-yl)pyrimidin-4-yl)methoxy)phenyl) propan-2-yl)benzonitrile (Compound 76) trifluoroacetate

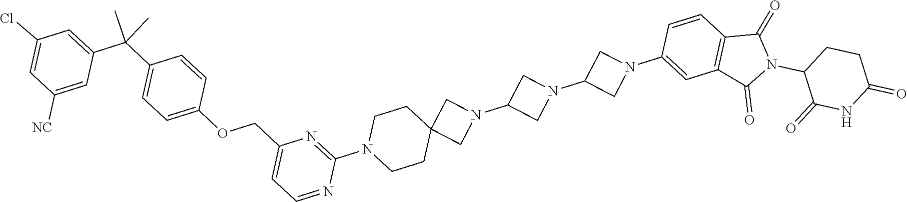 [SMILES: CC(C)(c1ccc(OCc2ccnc(N3CCC4(CC3)CN(C3CN(C5CN(c6ccc7c(c6)C(=O)N(C6CCC(=O)NC6=O)C7=O)C5)C3)C4)n2)cc1)c1cc(Cl)cc(C#N)c1]

The above crude 76d trifluoroacetate (60 mg) was dissolved in 4 mL of DMSO, and solid sodium bicarbonate (26 mg, 0.31 mmol) was added. The mixture was stirred at room temperature for 10 min, and then 0.2 mL of DIPEA and 2-(2,6-dioxopiperidin-3-yl)-5-fluoroisoindoline-1,3-dione (see WO 2017197056 for the synthetic method) (25 mg, 0.091 mmol) were added. The resulting mixture was warmed to 80° C. and reacted for 5 h. The reaction liquid was cooled to room temperature, and 50 mL of water was added. The mixture was filtered, and the solid (filter cake) was collected, washed with 20 mL of water, dissolved with 100 mL of DCM, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 76 trifluoroacetate (10 mg).

¹H NMR (400 MHz, DMSO-d₆) δ 11.06 (s, 1H), 8.46-8.28 (m, 1H), 7.95-7.45 (m, 4H), 7.27-6.55 (m, 7H), 5.16-4.87 (m, 3H), 4.38-4.06 (m, 3H), 4.06-3.60 (m, 15H), 3.00-2.79 (m, 1H), 2.70-2.51 (m, 2H), 2.13-1.92 (m, 1H), 1.92-1.74 (m, 4H), 1.65 (s, 6H).

LCMS m/z=854.3 [M+1]⁺.

### Example 77

### 3-chloro-5-(2-(4-((2-(4-((9-(2-(2,6-dioxopiperidin-3-yl)-1,3-dioxoisoindolin-5-yl)-3,9-diazaspiro[5.5]undecan-3-yl)methyl)piperidin-1-yl)pyrimidin-4-yl)methoxy)phenyl) propan-2-yl)benzonitrile (Compound 77) trifluoroacetate

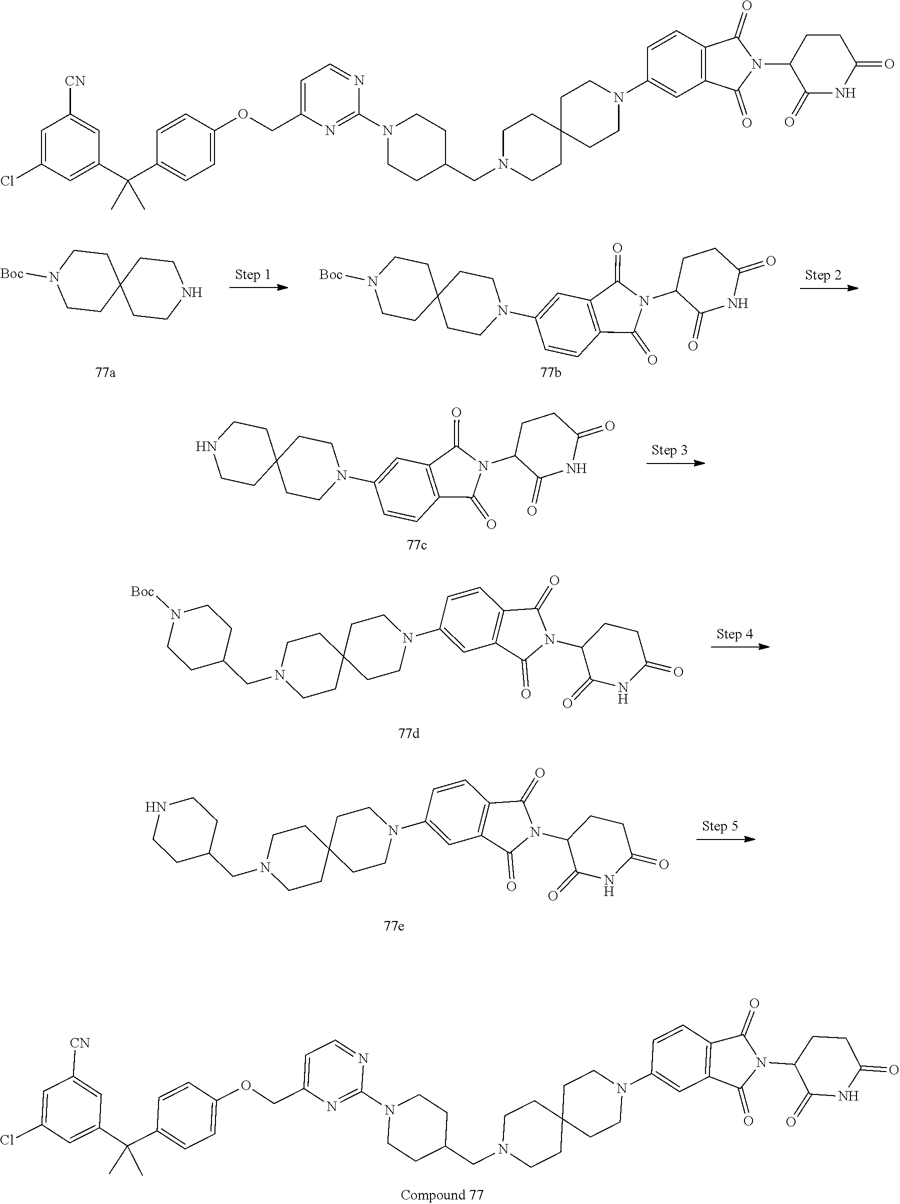

### Step 1: tert-butyl 9-(2-(2,6-dioxopiperidin-3-yl)-1,3-dioxoisoindolin-5-yl)-3,9-diazaspiro[5.5]undecane-3-carboxylate (77b)

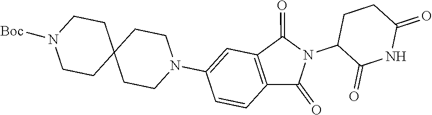 [SMILES: CC(C)(C)OC(=O)N1CCC2(CC1)CCN(c1ccc3c(c1)C(=O)N(C1CCC(=O)NC1=O)C3=O)CC2]

Tert-butyl 3,9-diazaspiro[5.5]undecane-3-carboxylate (77a) (850 mg, 3.34 mmol) was dissolved in 20 mL of DMSO, and 2 mL of DIPEA and 2-(2,6-dioxopiperidin-3-yl)-5-fluoroisoindoline-1,3-dione (see WO 2017197056 for the synthetic method) (1.38 g, 5.00 mmol) were added. The mixture was warmed to 80° C. and reacted for 5 h. The reaction liquid was cooled to room temperature, and 50 mL of water was added. The mixture was filtered, and the solid (filter cake) was collected, washed with 50 mL of water, dissolved with 100 mL of DCM, dried over anhydrous sodium sulfate, and concentrated under reduced pressure. The crude product was then separated and purified by silica gel column chromatography (dichloromethane/methanol (v/v)=20:1), to afford 77b (1.5 g, yield: 88%).

LCMS m/z=511.3 [M+1]⁺

### Step 2: 2-(2,6-dioxopiperidin-3-yl)-5-(3,9-diazaspiro[5.5]undecan-3-yl)isoindoline-1,3-dione (77c) trifluoroacetate

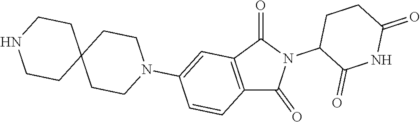 [SMILES: O=C1CCC(N2C(=O)c3ccc(N4CCC5(CCNCC5)CC4)cc3C2=O)C(=O)N1]

77b (250 mg, 0.49 mmol) was dissolved in 10 mL of DCM, and 4 mL of trifluoroacetic acid was added. The mixture was stirred at room temperature for 2 h. The reaction liquid was concentrated under reduced pressure, to afford crude 77c trifluoroacetate (290 mg).

LCMS m/z=411.2 [M+1]⁺

### Step 3: tert-butyl 4-((9-(2-(2,6-dioxopiperidin-3-yl)-1,3-dioxoisoindolin-5-yl)-3,9-diazaspiro[5.5]undecan-3-yl)methyl)piperidine-1-carboxylate (77d)

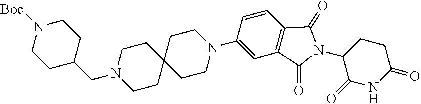 [SMILES: CC(C)(C)OC(=O)N1CCC(CN2CCC3(CC2)CCN(c2ccc4c(c2)C(=O)N(C2CCC(=O)NC2=O)C4=O)CC3)CC1]

The above crude 77c trifluoroacetate (290 mg) was dissolved in 15 mL of THF, and solid sodium bicarbonate (123 mg, 1.46 mmol) was added. The mixture was stirred at room temperature for 20 min, and then tert-butyl 4-formylpiperidine-1-carboxylate (210 mg, 0.98 mmol) and 0.1 mL of acetic acid were successively added. The mixture was stirred at room temperature for 60 min, and then sodium triacetoxyborohydride (210 mg, 0.99 mmol) was added. The resulting mixture was reacted at room temperature for 16 h. To the reaction system was slowly added 20 mL of saturated aqueous sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=15:1), to afford 77d (280 mg, two-step yield from compound 77b: 94%).

LCMS m/z=608.3 [M+1]⁺

### Step 4: 2-(2,6-dioxopiperidin-3-yl)-5-(9-(piperidin-4-ylmethyl)-3,9-diazaspiro[5.5]undecan-3-yl)isoindoline-1,3-dione (77e) trifluoroacetate

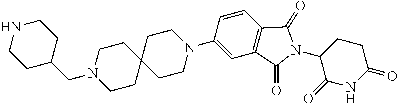 [SMILES: O=C1CCC(N2C(=O)c3ccc(N4CCC5(CCN(CC6CCNCC6)CC5)CC4)cc3C2=O)C(=O)N1]

77d (150 mg, 0.30 mmol) was dissolved in 10 mL of DCM, and 4 mL of trifluoroacetic acid was added. The mixture was stirred at room temperature for 2 h. The reaction liquid was concentrated under reduced pressure, to afford crude 77e trifluoroacetate (175 mg).

LCMS m/z=508.4 [M+1]⁺

### Step 5: 3-chloro-5-(2-(4-((2-(4-((9-(2-(2,6-dioxopiperidin-3-yl)-1,3-dioxoisoindolin-5-yl)-3,9-diazaspiro[5.5]undecan-3-yl)methyl)piperidin-1-yl)pyrimidin-4-yl)methoxy)phenyl) propan-2-yl)benzonitrile (Compound 77) trifluoroacetate

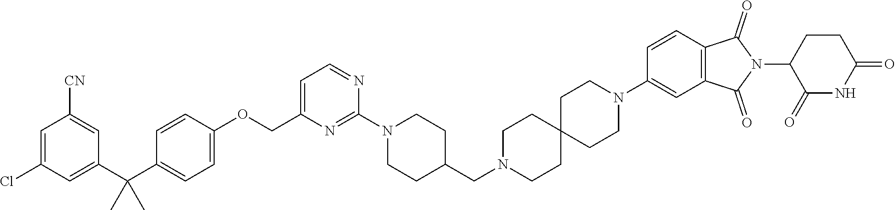 [SMILES: CC(C)(c1ccc(OCc2ccnc(N3CCC(CN4CCC5(CC4)CCN(c4ccc6c(c4)C(=O)N(C4CCC(=O)NC4=O)C6=O)CC5)CC3)n2)cc1)c1cc(Cl)cc(C#N)c1]

3-chloro-5-(2-(4-((2-chloropyrimidin-4-yl)methoxy)phenyl)propan-2-yl)benzonitrile (9d) (82 mg, 0.21 mmol) was dissolved in 20 mL of DMF, and the above crude 77e trifluoroacetate (175 mg) and solid potassium bicarbonate (103 mg, 1.03 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 77 trifluoroacetate (50 mg).

¹H NMR (400 MHz, DMSO-d₆) δ 11.06 (s, 1H), 8.36 (d, 1H), 7.86-7.82 (m, 1H), 7.72-7.60 (m, 2H), 7.57-7.49 (m, 1H), 7.38-7.22 (m, 2H), 7.21-7.13 (m, 2H), 6.99-6.91 (m, 2H), 6.69 (d, 1H), 5.07 (dd, 1H), 4.98 (s, 2H), 4.75-4.55 (m, 2H), 3.56-3.32 (m, 6H), 3.18-2.81 (m, 7H), 2.68-2.51 (m, 2H), 2.20-1.95 (m, 2H), 1.95-1.57 (m, 14H), 1.55-1.43 (m, 2H), 1.30-1.08 (m, 2H).

LCMS m/z=869.3 [M+1]⁺

### Example 78

### 3-chloro-5-(2-(4-((2-(4-((1′-(2-(2,6-dioxopiperidin-3-yl)-6-fluoro-1,3-dioxoisoindolin-5-yl)-[4,4′-bipiperidin]-1-yl)methyl)piperidin-1-yl)pyrimidin-4-yl)methoxy)phenyl)propan-2-yl)benzonitrile (Compound 78)

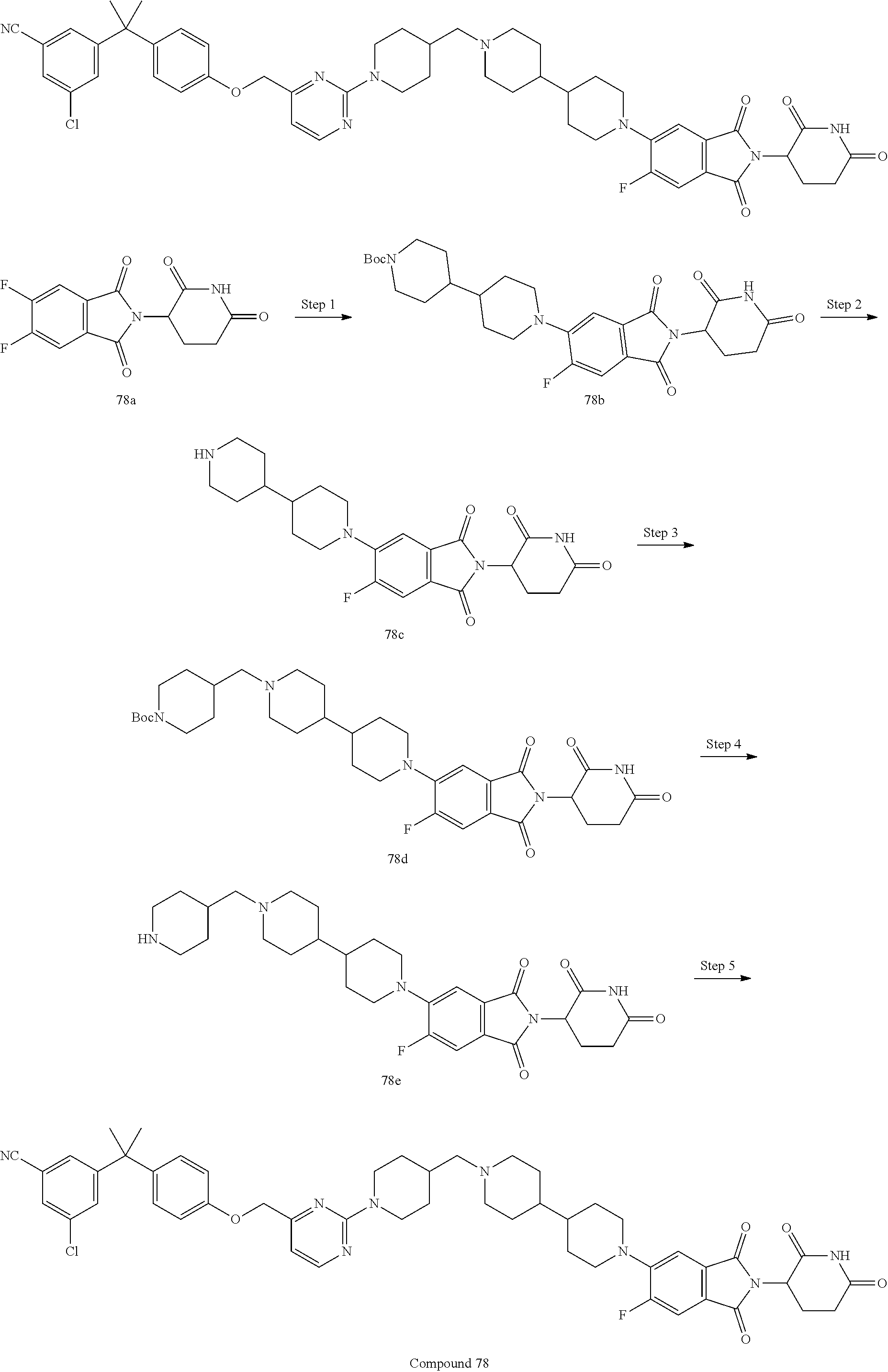

### Step 1: tert-butyl 1′(2-(2,6-dioxopiperidin-3-yl)-6-fluoro-1,3-dioxoisoindolin-5-yl)-[4,4′-bipiperidine]-1-carboxylate (78b)

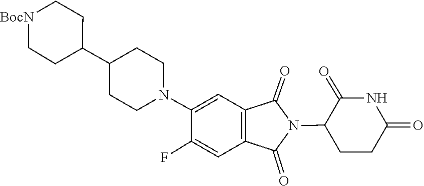 [SMILES: CC(C)(C)OC(=O)N1CCC(C2CCN(c3cc4c(cc3F)C(=O)N(C3CCC(=O)NC3=O)C4=O)CC2)CC1]

2-(2,6-dioxopiperidin-3-yl)-5,6-difluoroisoindoline-1,3-dione (78a) (see WO 2020239103 for the synthetic method) (500 mg, 1.7 mmol) was dissolved in 10 mL of DMF, and tert-butyl [4,4′-bipiperidine]-1-carboxylate (460 mg, 1.71 mmol) and solid potassium bicarbonate (266 mg, 2.66 mmol) were successively added. The mixture was warmed to 90° C. and reacted for 4 h. The reaction liquid was cooled to room temperature, and 30 mL of water was added. The mixture was extracted twice with 30 mL of ethyl acetate, and the organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=20:1), to afford 78b (900 mg, 98%).

LCMS m/z=543.2 [M+1]⁺

### Step 2: 5-([4,4′-bipiperidin]-1-yl)-2-(2,6-dioxopiperidin-3-yl)-6-fluoroisoindoline-1,3-dione (78c)

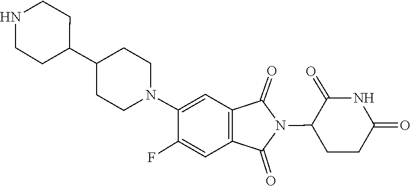 [SMILES: O=C1CCC(N2C(=O)c3cc(F)c(N4CCC(C5CCNCC5)CC4)cc3C2=O)C(=O)N1]

78b (900 mg, 1.66 mmol) was dissolved in 5 mL of dichloromethane, and 4 mL of trifluoroacetic acid was added. The mixture was stirred at room temperature for 4 h. The reaction system was concentrated under reduced pressure, and 50 mL of dichloromethane was added. The mixture was adjusted to pH 9 with a saturated sodium bicarbonate solution, extracted with 50 mL of dichloromethane, dried over anhydrous sodium sulfate, and concentrated under reduced pressure, to afford crude 78c (700 mg).

LCMS m/z=443.1 [M+1]⁺

### Step 3: tert-butyl 4-((1′-(2-(2,6-dioxopiperidin-3-yl)-6-fluoro-1,3-dioxoisoindolin-5-yl)-[4,4′-bipiperidin]-1-yl)methyl)piperidine-1-carboxylate (78d)

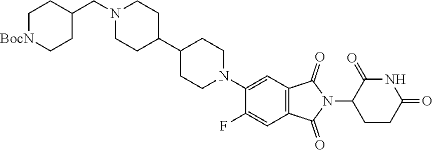 [SMILES: CC(C)(C)OC(=O)N1CCC(CN2CCC(C3CCN(c4cc5c(cc4F)C(=O)N(C4CCC(=O)NC4=O)C5=O)CC3)CC2)CC1]

The above crude 78c (300 mg) was dissolved in 15 mL of 1,2-dichloroethane, and tert-butyl 4-formylpiperidine-1-carboxylate (200 mg, 0.94 mmol) was added. The mixture was stirred at room temperature for 1 h, and then sodium triacetoxyborohydride (300 mg, 1.42 mmol) was added. The resulting mixture was stirred at room temperature for 16 h. To the reaction liquid was slowly added 20 mL of saturated sodium bicarbonate solution, and the mixture was extracted twice with 30 mL of dichloromethane. The organic phase was washed with 50 mL of saturated sodium chloride solution, dried over anhydrous sodium sulfate, and concentrated under reduced pressure. The crude product was then separated and purified by silica gel column chromatography (dichloromethane/methanol (v/v)=10:1), to afford 78d (300 mg, two-step yield from compound 78b: 66%).

LCMS m/z=640.4 [M+1]⁺

### Step 4: 2-(2,6-dioxopiperidin-3-yl)-5-fluoro-6-(1′-(piperidin-4-ylmethyl)-[4,4′-bipiperidin]-1-yl)isoindoline-1,3-dione (78e) trifluoroacetate

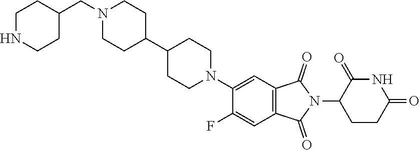 [SMILES: O=C1CCC(N2C(=O)c3cc(F)c(N4CCC(C5CCN(CC6CCNCC6)CC5)CC4)cc3C2=O)C(=O)N1]

78d (300 mg, 0.47 mmol) was dissolved in 2 mL of DCM, and 1 mL of trifluoroacetic acid was added. The mixture was stirred at room temperature for 4 h. The reaction system was concentrated under reduced pressure, to afford crude 78e trifluoroacetate (310 mg).

LCMS m/z=540.2 [M+1]⁺

### Step 5: 3-chloro-5-(2-(4-((2-(4-((1′-(2-(2,6-dioxopiperidin-3-yl)-6-fluoro-1,3-dioxoisoindolin-5-yl)-[4,4′-bipiperidin]-1-yl)methyl)piperidin-1-yl)pyrimidin-4-yl)methoxy)phenyl)propan-2-yl)benzonitrile (Compound 78)

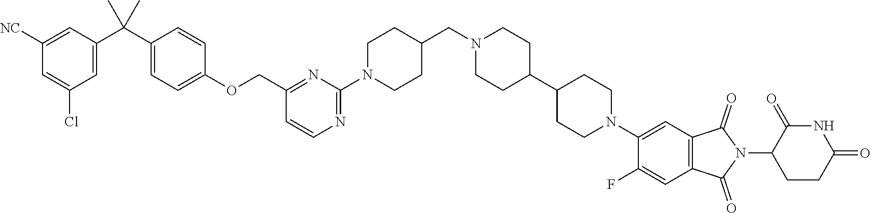 [SMILES: CC(C)(c1ccc(OCc2ccnc(N3CCC(CN4CCC(C5CCN(c6cc7c(cc6F)C(=O)N(C6CCC(=O)NC6=O)C7=O)CC5)CC4)CC3)n2)cc1)c1cc(Cl)cc(C#N)c1]

The above crude 78e trifluoroacetate (310 mg) was dissolved in 15 mL of DMF, and 3-chloro-5-(2-(4-((2-chloropyrimidin-4-yl)methoxy)phenyl)propan-2-yl)benzonitrile (9d) (200 mg, 0.5 mmol) and solid potassium bicarbonate (500 mg, 5 mmol) were successively added. The mixture was warmed to 90° C. and reacted for 2 h. The reaction liquid was cooled to room temperature, and 30 mL of water was added. The mixture was extracted twice with 30 mL of ethyl acetate, and the organic phase was washed with 50 mL of saturated sodium chloride solution, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min). Then the preparative liquid was adjusted to pH 9 with a saturated sodium bicarbonate solution and extracted twice with 50 mL of ethyl acetate. The organic phase was washed twice with 50 mL of purified water, dried over anhydrous sodium sulfate, and concentrated under reduced pressure, to afford compound 78 (100 mg, two-step yield from compound 78d: 24%).

¹H NMR (400 MHz, CDCl₃) δ 8.29 (d, 1H), 8.06 (br.s, 1H), 7.50-7.30 (m, 5H), 7.13-7.02 (m, 2H), 6.93-6.84 (m, 2H), 6.66 (d, 1H), 5.00-4.88 (m, 3H), 4.84-4.68 (m, 2H), 3.75-3.63 (m, 2H), 3.07-2.64 (m, 9H), 2.35-2.07 (m, 3H), 1.98-1.77 (m, 6H), 1.77-1.55 (m, 9H), 1.50-1.05 (m, 8H).

LCMS m/z=451.3 [M/2+1]⁺

### Example 79

### 3-chloro-5-(2-(4-((2-(1′-((1-(2-(2,6-dioxopiperidin-3-yl)-1,3-dioxoisoindolin-5-yl)azetidin-3-yl)methyl)-[4,4′-bipiperidin]-1-yl)pyrimidin-4-yl)methoxy)phenyl)propan-2-yl)benzonitrile (Compound 79)

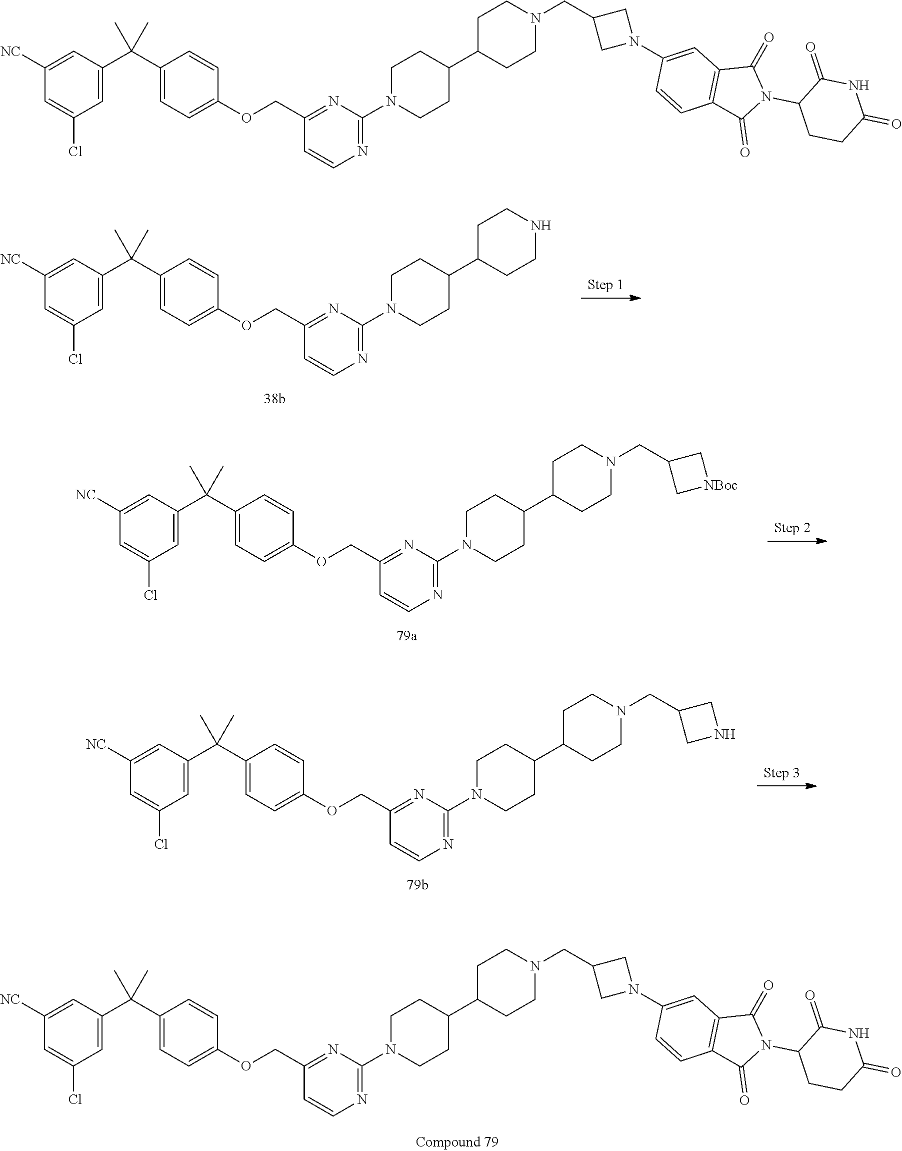 [SMILES: CC(C)(c1ccc(OCc2ccnc(N3CCC(C4CCN(CC5CN(c6ccc7c(c6)C(=O)N(C6CCC(=O)NC6=O)C7=O)C5)CC4)CC3)n2)cc1)c1cc(Cl)cc(C#N)c1 ; CC(C)(c1ccc(OCc2ccnc(N3CCC(C4CCNCC4)CC3)n2)cc1)c1cc(Cl)cc(C#N)c1 ; CC(C)(C)OC(=O)N1CC(CN2CCC(C3CCN(c4nccc(COc5ccc(C(C)(C)c6cc(Cl)cc(C#N)c6)cc5)n4)CC3)CC2)C1 ; CC(C)(c1ccc(OCc2ccnc(N3CCC(C4CCN(CC5CNC5)CC4)CC3)n2)cc1)c1cc(Cl)cc(C#N)c1 ; CC(C)(c1ccc(OCc2ccnc(N3CCC(C4CCN(CC5CN(c6ccc7c(c6)C(=O)N(C6CCC(=O)NC6=O)C7=O)C5)CC4)CC3)n2)cc1)c1cc(Cl)cc(C#N)c1]

### Step 1: tert-butyl 3-((1′-(4-((4-(2-(3-chloro-5-cyanophenyl)propan-2-yl)phenoxy)methyl)pyrimidin-2-yl)-[4,4′-bipiperidin]-1-yl)methyl)azetidine-1-carboxylate (79a)

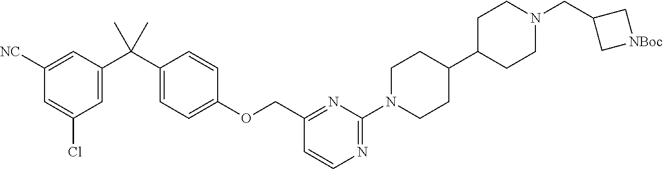 [SMILES: CC(C)(C)OC(=O)N1CC(CN2CCC(C3CCN(c4nccc(COc5ccc(C(C)(C)c6cc(Cl)cc(C#N)c6)cc5)n4)CC3)CC2)C1]

The above crude 3-(2-(4-((2-([4,4′-bipiperidin]-1-yl)pyrimidin-4-yl)methoxy)phenyl)propan-2-yl)-5-chlorobenzonitrile (38b) (350 mg) was dissolved in 25 mL of 1,2-dichloroethane, and tert-butyl 3-formylazetidine-1-carboxylate (240 mg, 1.30 mmol) was added. The mixture was stirred at room temperature for 1 h, and then sodium triacetoxyborohydride (420 mg, 1.98 mmol) was added. The resulting mixture was reacted at room temperature for 16 h. To the reaction liquid was slowly added 50 mL of saturated sodium bicarbonate solution, and the mixture was extracted twice with 100 mL of dichloromethane. The organic phase was washed with 50 mL of saturated sodium chloride solution, dried over anhydrous sodium sulfate, and concentrated under reduced pressure. The crude product was then separated and purified by silica gel column chromatography (dichloromethane/methanol (v/v)=1:0-10:1), to afford 79a (400 mg, two-step yield from compound 38a: 77%).

LCMS m/z=699.4 [M+1]⁺

### Step 2: 3-(2-(4-((2-(1′-(azetidin-3-ylmethyl)-[4,4′-bipiperidin]-1-yl)pyrimidin-4-yl)methoxy)phenyl)propan-2-yl)-5-chlorobenzonitrile (79b) trifluoroacetate

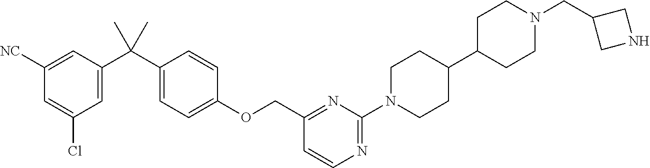 [SMILES: CC(C)(c1ccc(OCc2ccnc(N3CCC(C4CCN(CC5CNC5)CC4)CC3)n2)cc1)c1cc(Cl)cc(C#N)c1]

79a (0.38 g, 0.54 mmol) was dissolved in 5 mL of DCM, and 2 mL of trifluoroacetic acid was added. The mixture was reacted at room temperature for 2 h. The reaction system was concentrated under reduced pressure, to afford crude 79b trifluoroacetate (0.39 g).

LCMS m/z=599.2 [M+1]⁺

### Step 3: 3-chloro-5-(2-(4-((2-(1′-((1-(2-(2,6-dioxopiperidin-3-yl)-1,3-dioxoisoindolin-5-yl)azetidin-3-yl)methyl)-[4,4′-bipiperidin]-1-yl)pyrimidin-4-yl)methoxy)phenyl)propan-2-yl)benzonitrile (compound 79)

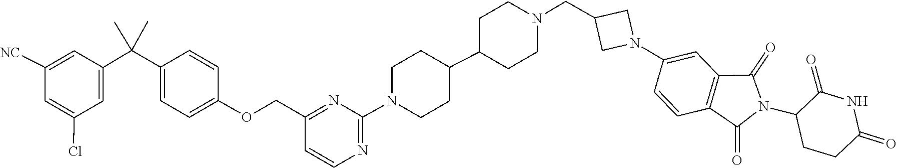 [SMILES: CC(C)(c1ccc(OCc2ccnc(N3CCC(C4CCN(CC5CN(c6ccc7c(c6)C(=O)N(C6CCC(=O)NC6=O)C7=O)C5)CC4)CC3)n2)cc1)c1cc(Cl)cc(C#N)c1]

The above crude 79b trifluoroacetate (0.39 g) was dissolved in 25 mL of DMSO, and solid potassium bicarbonate was added to adjust pH to 9. 1 mL of DIPEA and 2-(2,6-dioxopiperidin-3-yl)-5-fluoroisoindoline-1,3-dione (see WO 2017197056 for the synthetic method) (150 mg, 0.54 mmol) were added, and the mixture was warmed to 80° C. and reacted for 8 h. The reaction liquid was cooled to room temperature, and 30 mL of saturated sodium bicarbonate solution was slowly added. The mixture was extracted twice with 60 mL of ethyl acetate, and the organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 m filter membrane, to prepare into a sample liquid. Mobile phase system: acetonitrile/water (containing 0.1% TFA). Gradient elution method: gradient elution with acetonitrile from 5% to 60% (elution time: 15 min). Then the preparative liquid was adjusted to pH 9 with a saturated sodium bicarbonate solution and extracted twice with 50 mL of ethyl acetate. The organic phase was washed twice with 50 mL of purified water, dried over anhydrous sodium sulfate, and concentrated under reduced pressure, to afford compound 79 (220 mg, two-step yield from compound 79a: 48%).

¹H NMR (400 MHz, CDCl₃) δ 8.53 (br.s, 1H), 8.29 (d, 1H), 7.64 (d, 1H), 7.47-7.35 (m, 3H), 7.12-7.05 (m, 2H), 6.93-6.80 (m, 3H), 6.66 (d, 1H), 6.49 (dd, 1H), 4.98-4.76 (m, 5H), 4.25-4.05 (m, 2H), 3.79-3.64 (m, 2H), 3.25-2.50 (m, 10H), 2.18-2.06 (m, 1H), 2.04-1.88 (m, 1H), 1.85-1.52 (m, 12H), 1.47-1.05 (m, 5H).

LCMS m/z=428.3 [M/2+1]⁺

### Example 80

### 3-chloro-5-(2-(4-((2-(4-((2-(2,6-dioxopiperidin-3-yl)-1,3-dioxoisoindolin-5-yl)ethynyl)piperidin-1-yl)pyrimidin-4-yl)methoxy)phenyl)propan-2-yl)benzonitrile (Compound 80) trifluoroacetate

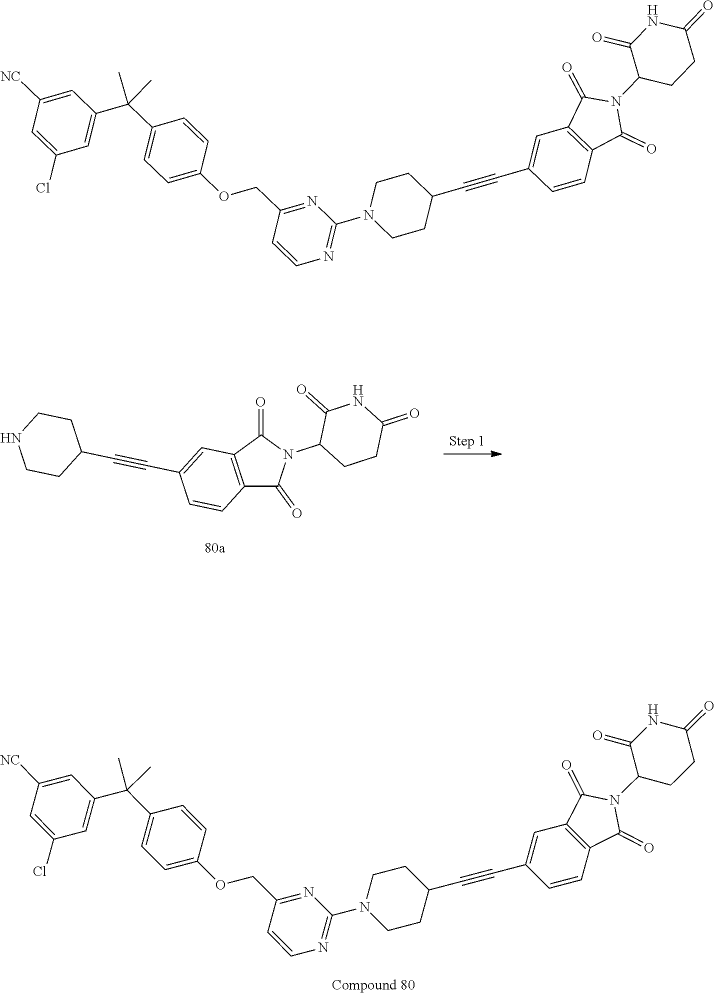 [SMILES: CC(C)(c1ccc(OCc2ccnc(N3CCC(C#Cc4ccc5c(c4)C(=O)N(C4CCC(=O)NC4=O)C5=O)CC3)n2)cc1)c1cc(Cl)cc(C#N)c1 ; O=C1CCC(N2C(=O)c3ccc(C#CC4CCNCC4)cc3C2=O)C(=O)N1 ; CC(C)(c1ccc(OCc2ccnc(N3CCC(C#Cc4ccc5c(c4)C(=O)N(C4CCC(=O)NC4=O)C5=O)CC3)n2)cc1)c1cc(Cl)cc(C#N)c1]

2-(2,6-dioxopiperidin-3-yl)-5-(piperidin-4-yl)ethynyl)isoindoline-1,3-dione (80a) trifluoroacetate (see CN112390785 for the synthetic method) (0.15 g) was added to 4 mL of DMSO, and solid sodium bicarbonate (52 mg, 0.52 mmol) was added. The mixture was stirred at room temperature for 1 h, and then 0.5 mL of DIPEA and 3-chloro-5-(2-(4-((2-chloropyrimidin-4-yl)methoxy)phenyl)propan-2-yl)benzonitrile (9d) (0.15 g, 0.38 mmol) were successively added. The resulting mixture was warmed to 80° C. and reacted for 5 h. The reaction liquid was cooled to room temperature, and 30 mL of water was added. The mixture was extracted with 50 mL of ethyl acetate, and the organic phase was washed with 20 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 80 trifluoroacetate (30 mg).

¹H NMR (400 MHz, CD₃OD) δ 8.29 (d, 1H), 7.89-7.80 (m, 3H), 7.61-7.56 (m, 1H), 7.53-7.46 (m, 2H), 7.22-7.14 (m, 2H), 7.00-6.92 (m, 2H), 6.87 (d, 1H), 5.18-5.05 (m, 3H), 4.33-4.19 (m, 2H), 3.71-3.55 (m, 2H), 3.15-3.00 (m, 1H), 2.96-2.62 (m, 3H), 2.23-2.09 (m, 1H), 2.09-1.97 (m, 2H), 1.85-1.70 (m, 2H), 1.67 (s, 6H).

LCMS m/z=727.1 [M+1]⁺

### Example 81

### 3-chloro-5-(2-(4-((2-(5-(1-(1-(2-(2,6-dioxopiperidin-3-yl)-1,3-dioxoisoindolin-5-yl)azetidin-3-yl)piperidin-4-yl)hexahydropyrrolo[3,4-c]pyrrol-2(1H)-yl)pyrimidin-4-yl)methoxy)phenyl)propan-2-yl)benzonitrile (Compound 81) trifluoroacetate

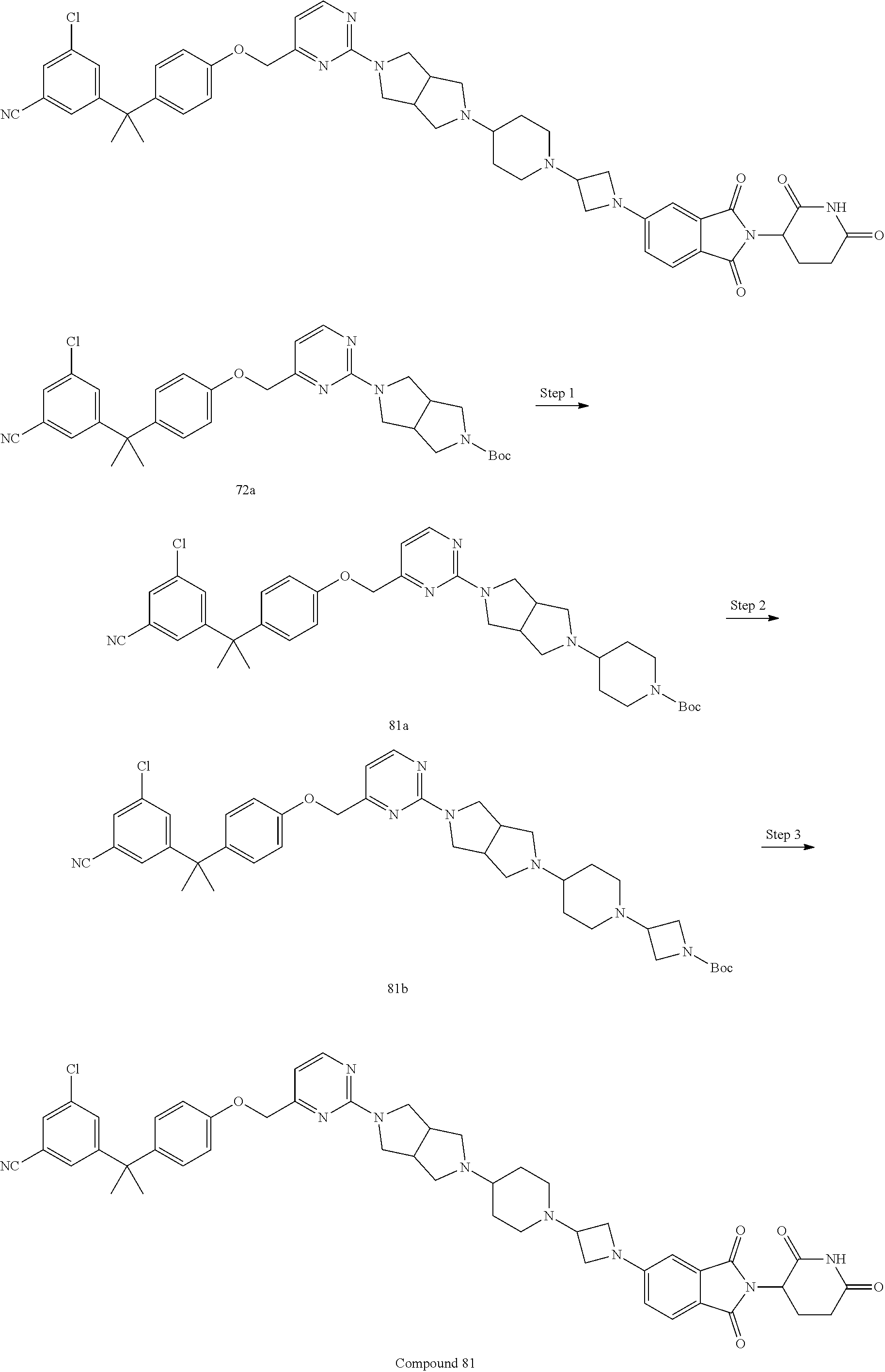 [SMILES: CC(C)(c1ccc(OCc2ccnc(N3CC4CN(C5CCN(C6CN(c7ccc8c(c7)C(=O)N(C7CCC(=O)NC7=O)C8=O)C6)CC5)CC4C3)n2)cc1)c1cc(Cl)cc(C#N)c1 ; CC(C)(C)OC(=O)N1CC2CN(c3nccc(COc4ccc(C(C)(C)c5cc(Cl)cc(C#N)c5)cc4)n3)CC2C1 ; CC(C)(C)OC(=O)N1CCC(N2CC3CN(c4nccc(COc5ccc(C(C)(C)c6cc(Cl)cc(C#N)c6)cc5)n4)CC3C2)CC1 ; CC(C)(C)OC(=O)N1CC(N2CCC(N3CC4CN(c5nccc(COc6ccc(C(C)(C)c7cc(Cl)cc(C#N)c7)cc6)n5)CC4C3)CC2)C1 ; CC(C)(c1ccc(OCc2ccnc(N3CC4CN(C5CCN(C6CN(c7ccc8c(c7)C(=O)N(C7CCC(=O)NC7=O)C8=O)C6)CC5)CC4C3)n2)cc1)c1cc(Cl)cc(C#N)c1]

### Step 1: tert-butyl 4-(5-(4-((4-(2-(3-chloro-5-cyanophenyl)propan-2-yl)phenoxy)methyl) pyrimidin-2-yl) hexahydropyrrolo[3,4-c]pyrrol-2(1H)-yl)piperidine-1-carboxylate (81a)

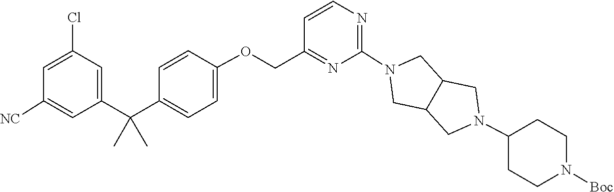 [SMILES: CC(C)(C)OC(=O)N1CCC(N2CC3CN(c4nccc(COc5ccc(C(C)(C)c6cc(Cl)cc(C#N)c6)cc5)n4)CC3C2)CC1]

72a (170 mg, 0.3 mmol) was dissolved in 4 mL of dichloromethane, and 2 mL trifluoroacetic acid was added. The mixture was stirred at room temperature for 2 h. The reaction system was concentrated under reduced pressure, and 10 mL of tetrahydrofuran and solid sodium bicarbonate (76 mg, 0.9 mmol) were added. The mixture was stirred at room temperature for 0.5 h, and then N-BOC-piperidone (120 mg, 0.6 mmol) was added. The mixture was stirred at room temperature for further 0.5 h, and then sodium triacetoxyborohydride (320 mg, 1.5 mmol) was added. The resulting mixture was reacted at room temperature for 16 h. To the reaction liquid was slowly added 20 mL of saturated sodium bicarbonate solution, and the mixture was extracted with 50 mL of dichloromethane. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 81a (161 mg, yield: 82%).

LCMS m/z=657.3 [M+1]⁺

### Step 2: tert-butyl 3-(4-(5-(4-((4-(2-(3-chloro-5-cyanophenyl)propan-2-yl)phenoxy)methyl)pyrimidin-2-yl)hexahydropyrrolo[3,4-c]pyrrol-2(1H)-yl)piperidin-1-yl)azetidine-1-carboxylate (81b)

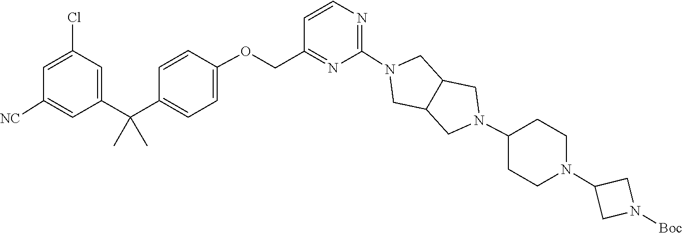 [SMILES: CC(C)(C)OC(=O)N1CC(N2CCC(N3CC4CN(c5nccc(COc6ccc(C(C)(C)c7cc(Cl)cc(C#N)c7)cc6)n5)CC4C3)CC2)C1]

81a (79 mg, 0.12 mmol) was dissolved in 2 mL of dichloromethane, and 1 mL of trifluoroacetic acid was added. The mixture was stirred at room temperature for 2 h. The reaction system was concentrated under reduced pressure, and 10 mL of tetrahydrofuran and solid sodium bicarbonate (30 mg, 0.36 mmol) were added. The mixture was stirred at room temperature for 0.5 h, and then N-BOC-azetidinone (41 mg, 0.24 mmol) was added. The mixture was stirred at room temperature for further 0.5 h, and then sodium triacetoxyborohydride (130 mg, 0.61 mmol) was added. The resulting mixture was reacted at room temperature for 16 h. To the reaction liquid was slowly added 10 mL of saturated sodium bicarbonate solution, and the mixture was extracted with 30 mL of dichloromethane. The organic phase was washed with 30 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 81b (74 mg, yield: 87%).

LCMS m/z=712.3 [M+1]⁺

### Step 3: 3-chloro-5-(2-(4-((2-(5-(1-(1-(2-(2,6-dioxopiperidin-3-yl)-1,3-dioxoisoindolin-5-yl)azetidin-3-yl)piperidin-4-yl)hexahydropyrrolo[3,4-c]pyrrol-2(1H)-yl)pyrimidin-4-yl)methoxy)phenyl)propan-2-yl)benzonitrile (Compound 81) trifluoroacetate

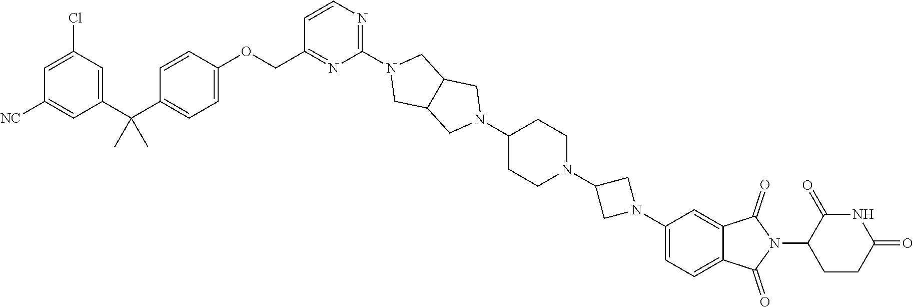 [SMILES: CC(C)(c1ccc(OCc2ccnc(N3CC4CN(C5CCN(C6CN(c7ccc8c(c7)C(=O)N(C7CCC(=O)NC7=O)C8=O)C6)CC5)CC4C3)n2)cc1)c1cc(Cl)cc(C#N)c1]

81b (74 mg, 0.1 mmol) was dissolved in 2 mL of DCM, and 0.7 mL of trifluoroacetic acid was added. The mixture was stirred at room temperature for 2 h. The reaction system was concentrated under reduced pressure to afford an oil. The oil was dissolved in 5 mL of DMSO, and solid sodium bicarbonate (34 mg, 0.4 mmol) was added. The mixture was stirred at room temperature for 0.5 h, and then 0.15 mL of DIPEA and 2-(2,6-dioxopiperidin-3-yl)-5-fluoroisoindoline-1,3-dione (see WO 2017197056 for the synthetic method) (28 mg, 0.1 mmol) were added. The resulting mixture was warmed to 85° C. and reacted for 3 h. The reaction liquid was cooled to room temperature, and 20 mL of water was added to precipitate out a large number of yellow solids. The yellow solids were filtered by suction, dissolved in 50 mL of mixed solvent (dichloromethane/methanol (v/v)=10:1), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 81 trifluoroacetate (34 mg).

¹H NMR (400 MHz, DMSO-d₆) δ 11.06 (s, 1H), 8.39 (d, 1H), 7.88-7.82 (m, 1H), 7.75-7.60 (m, 2H), 7.56-7.49 (m, 1H), 7.21-7.13 (m, 2H), 6.98-6.90 (m, 2H), 6.89-6.83 (m, 1H), 6.80-6.68 (m, 2H), 5.12-4.93 (m, 3H), 4.37-3.80 (m, 10H), 3.55-2.80 (m, 11H), 2.70-2.50 (m, 2H), 2.35-2.11 (m, 2H), 2.10-1.94 (m, 1H), 1.90-1.55 (m, 8H).

LCMS m/z=868.3 [M+1]⁺

### Example 82

### 3-chloro-5-(2-(4-((2-(5-(1-((1-(2-(2,6-dioxopiperidin-3-yl)-1,3-dioxoisoindolin-5-yl) piperidin-4-yl)methyl)piperidin-4-yl)hexahydropyrrolo[3,4-c]pyrrol-2(1H)-yl)pyrimidin-4-yl)methoxy)phenyl)propan-2-yl)benzonitrile (Compound 82) trifluoroacetate

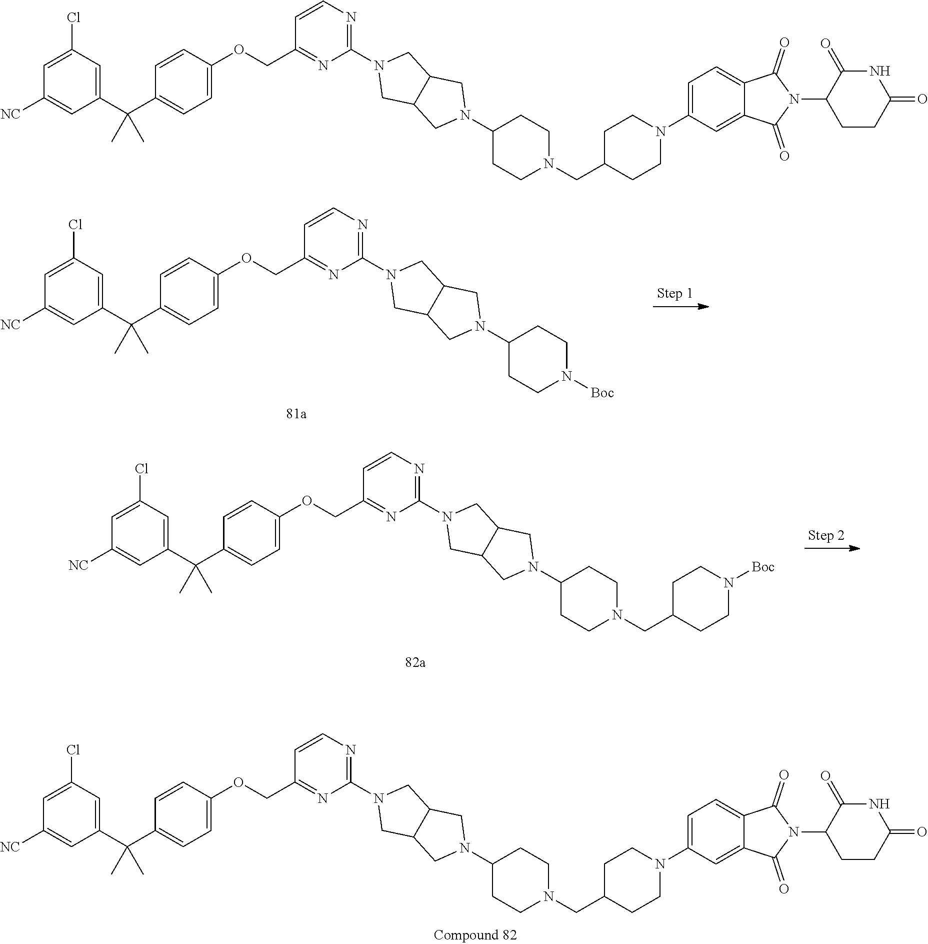 [SMILES: CC(C)(c1ccc(OCc2ccnc(N3CC4CN(C5CCN(CC6CCN(c7ccc8c(c7)C(=O)N(C7CCC(=O)NC7=O)C8=O)CC6)CC5)CC4C3)n2)cc1)c1cc(Cl)cc(C#N)c1 ; CC(C)(C)OC(=O)N1CCC(N2CC3CN(c4nccc(COc5ccc(C(C)(C)c6cc(Cl)cc(C#N)c6)cc5)n4)CC3C2)CC1 ; CC(C)(C)OC(=O)N1CCC(CN2CCC(N3CC4CN(c5nccc(COc6ccc(C(C)(C)c7cc(Cl)cc(C#N)c7)cc6)n5)CC4C3)CC2)CC1 ; CC(C)(c1ccc(OCc2ccnc(N3CC4CN(C5CCN(CC6CCN(c7ccc8c(c7)C(=O)N(C7CCC(=O)NC7=O)C8=O)CC6)CC5)CC4C3)n2)cc1)c1cc(Cl)cc(C#N)c1]

### Step 1: tert-butyl 4-((4-(5-(4-((4-(2-(3-chloro-5-cyanophenyl)propan-2-yl)phenoxy)methyl)pyrimidin-2-yl)hexahydropyrrolo[3,4-c]pyrrol-2(1H)-yl)piperidin-1-yl)methyl)piperidine-1-carboxylate (82a)

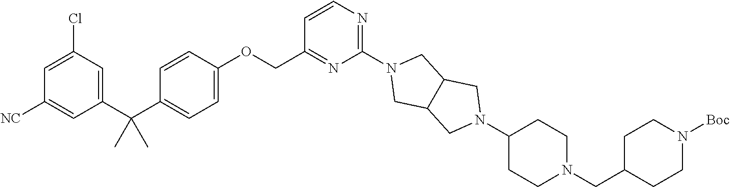 [SMILES: CC(C)(C)OC(=O)N1CCC(CN2CCC(N3CC4CN(c5nccc(COc6ccc(C(C)(C)c7cc(Cl)cc(C#N)c7)cc6)n5)CC4C3)CC2)CC1]

81a (79 mg, 0.12 mmol) was dissolved in 4 mL of dichloromethane, and 2 mL of trifluoroacetic acid was added. The mixture was stirred at room temperature for 2 h. The reaction system was concentrated under reduced pressure, and 10 mL of tetrahydrofuran and sodium bicarbonate (30 mg, 0.36 mmol) were added. The mixture was stirred at room temperature for 0.5 h, and then N-tertbutoxycarbonylpiperidine-4-carbaldehyde (51 mg, 0.24 mmol) was added. The mixture was stirred at room temperature for further 0.5 h, and then sodium triacetoxyborohydride (130 mg, 0.61 mmol) was added. The resulting mixture was reacted at room temperature for 16 h. To the reaction liquid was slowly added 20 mL of saturated sodium bicarbonate solution, and the mixture was extracted with 50 mL of dichloromethane. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 82a (88 mg, yield: 97%).

### Step 2: 3-chloro-5-(2-(4-((2-(5-(1-((1-(2-(2,6-dioxopiperidin-3-yl)-1,3-dioxoisoindolin-5-yl)piperidin-4-yl)methyl)piperidin-4-yl)hexahydropyrrolo[3,4-c]pyrrol-2(1H)-yl)pyrimidin-4-yl)methoxy)phenyl)propan-2-yl)benzonitrile (Compound 82) trifluoroacetate

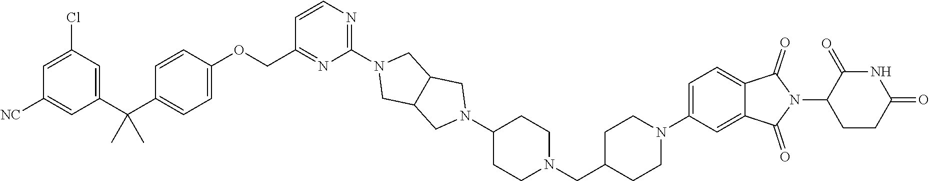 [SMILES: CC(C)(c1ccc(OCc2ccnc(N3CC4CN(C5CCN(CC6CCN(c7ccc8c(c7)C(=O)N(C7CCC(=O)NC7=O)C8=O)CC6)CC5)CC4C3)n2)cc1)c1cc(Cl)cc(C#N)c1]

82a (88 mg, 0.12 mmol) was dissolved in 2 mL of DCM, and 1 mL of trifluoroacetic acid was added. The mixture was stirred at room temperature for 2 h. The reaction system was concentrated under reduced pressure to afford an oil. The oil was dissolved in 5 mL of DMSO, and solid sodium bicarbonate (40 mg, 0.48 mmol) was added. The mixture was stirred at room temperature for 0.5 h, and then 0.1 mL of DIPEA and 2-(2,6-dioxopiperidin-3-yl)-5-fluoroisoindoline-1,3-dione (see WO 2017197056 for the synthetic method) (33 mg, 0.12 mmol) were added. The resulting mixture was warmed to 85° C. and reacted for 3 h. The reaction liquid was cooled to room temperature, and 25 mL of water was added to precipitate out a large number of yellow solids. The yellow solids were filtered by suction, dissolved in 50 mL of mixed solvent (dichloromethane/methanol (v/v)=10:1), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 82 trifluoroacetate (25 mg).

¹H NMR (400 MHz, DMSO-d₆) δ 11.06 (s, 1H), 8.39 (d, 1H), 7.87-7.83 (m, 1H), 7.70-7.62 (m, 2H), 7.55-7.50 (m, 1H), 7.38-7.32 (m, 1H), 7.30-7.22 (m, 1H), 7.22-7.13 (m, 2H), 7.00-6.88 (m, 2H), 6.81-6.71 (m, 1H), 5.11-4.92 (m, 3H), 4.15-3.55 (m, 12H), 3.40-3.20 (m, 1H), 3.17-2.75 (m, 9H), 2.69-2.50 (m, 2H), 2.36-1.76 (m, 8H), 1.64 (s, 6H), 1.35-1.16 (m, 2H).

LCMS m/z=455.9 [(M/2+1]⁺.

### Example 83

### 3-chloro-5-(2-(4-((2-(9-((1-(2-(2,6-dioxopiperidin-3-yl)-1,3-dioxoisoindolin-5-yl)pyrrolidin-3-yl)methyl)-3,9-diazaspiro[5.5]undecan-3-yl)pyrimidin-4-yl)methoxy)phenyl)propan-2-yl)benzonitrile (Compound 83)

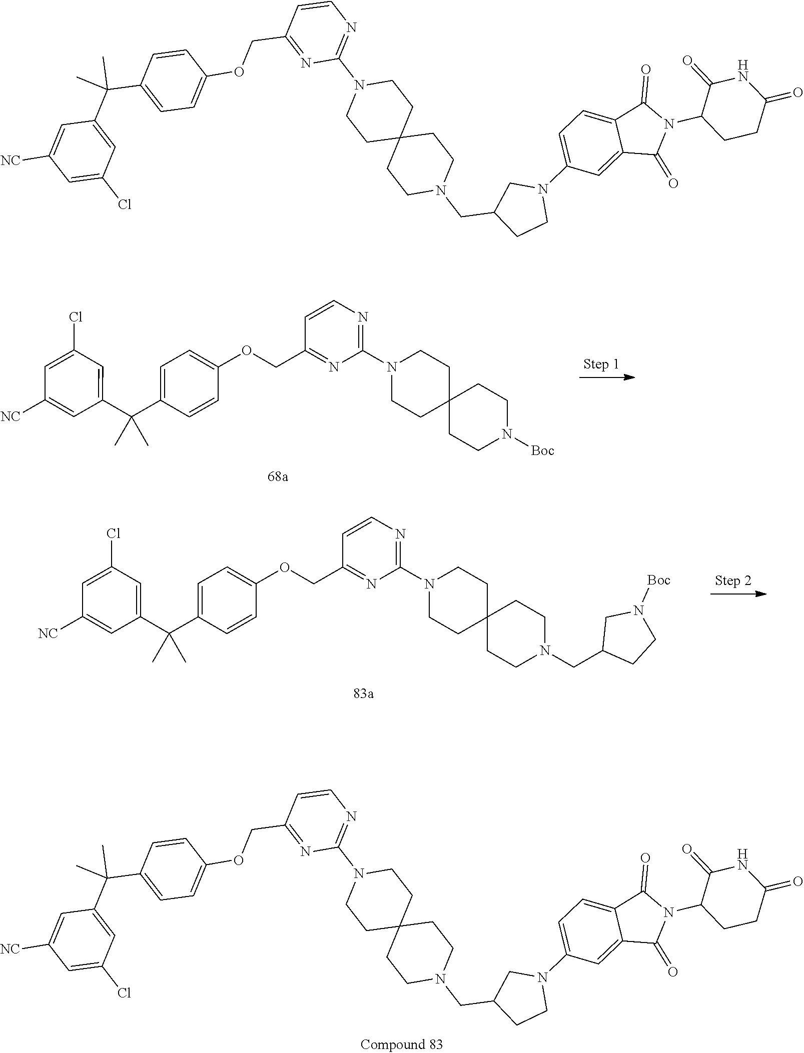 [SMILES: CC(C)(c1ccc(OCc2ccnc(N3CCC4(CCN(CC5CCN(c6ccc7c(c6)C(=O)N(C6CCC(=O)NC6=O)C7=O)C5)CC4)CC3)n2)cc1)c1cc(Cl)cc(C#N)c1 ; CC(C)(C)OC(=O)N1CCC2(CC1)CCN(c1nccc(COc3ccc(C(C)(C)c4cc(Cl)cc(C#N)c4)cc3)n1)CC2 ; CC(C)(C)OC(=O)N1CCC(CN2CCC3(CC2)CCN(c2nccc(COc4ccc(C(C)(C)c5cc(Cl)cc(C#N)c5)cc4)n2)CC3)C1 ; CC(C)(c1ccc(OCc2ccnc(N3CCC4(CCN(CC5CCN(c6ccc7c(c6)C(=O)N(C6CCC(=O)NC6=O)C7=O)C5)CC4)CC3)n2)cc1)c1cc(Cl)cc(C#N)c1]

### Step 1: tert-butyl 3-((9-(4-((4-(2-(3-chloro-5-cyanophenyl)propan-2-yl)phenoxy)methyl)pyrimidin-2-yl)-3,9-diazaspiro[5.5]undecan-3-yl)methyl)pyrrolidine-1-carboxylate (83a)

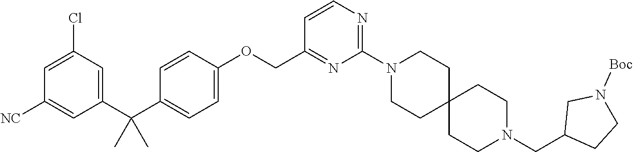 [SMILES: CC(C)(C)OC(=O)N1CCC(CN2CCC3(CC2)CCN(c2nccc(COc4ccc(C(C)(C)c5cc(Cl)cc(C#N)c5)cc4)n2)CC3)C1]

68a (200 mg, 0.32 mmol) was dissolved in 6 mL of dichloromethane, and 4 mL of trifluoroacetic acid was added. The mixture was stirred at room temperature for 2 h. The reaction system was concentrated under reduced pressure, and 8 mL of tetrahydrofuran and solid sodium bicarbonate (81 mg, 0.96 mmol) were added. The mixture was stirred at room temperature for 0.5 h, and then 1-Boc-3-pyrrolidinecarbaldehyde (190 mg, 0.95 mmol) was added. The mixture was stirred at room temperature for 0.5 h, and then sodium triacetoxyborohydride (340 mg, 1.6 mmol) was added. The resulting mixture was reacted at room temperature for 16 h. To the reaction liquid was slowly added 50 mL of saturated sodium bicarbonate solution, and the mixture was extracted with 100 mL of dichloromethane. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=1:0-9:1), to afford 83a (105 mg, yield: 47%).

### Step 2: 3-chloro-5-(2-(4-((2-(9-((1-(2-(2,6-dioxopiperidin-3-yl)-1,3-dioxoisoindolin-5-yl)pyrrolidin-3-yl)methyl)-3,9-diazaspiro[5.5]undecan-3-yl)pyrimidin-4-yl)methoxy)phenyl)propan-2-yl)benzonitrile (Compound 83)

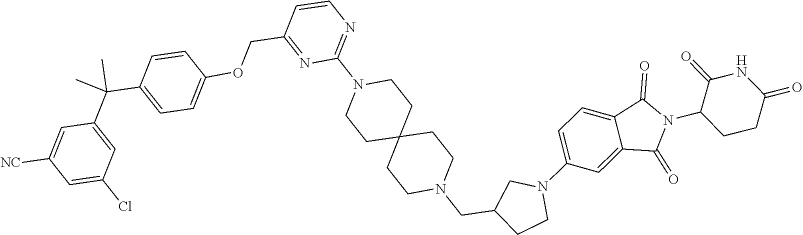 [SMILES: CC(C)(c1ccc(OCc2ccnc(N3CCC4(CCN(CC5CCN(c6ccc7c(c6)C(=O)N(C6CCC(=O)NC6=O)C7=O)C5)CC4)CC3)n2)cc1)c1cc(Cl)cc(C#N)c1]

83a (101 mg, 0.14 mmol) was dissolved in 4 mL of DCM, and 1 mL of trifluoroacetic acid was added. The mixture was stirred at room temperature for 2 h and concentrated under reduced pressure to afford an oil. The oil was dissolved in 8 mL of DMSO, and solid sodium bicarbonate (35 mg, 0.42 mmol) was added. The mixture was stirred at room temperature for 0.5 h, and 0.15 mL of DIPEA and 2-(2,6-dioxopiperidin-3-yl)-5-fluoroisoindoline-1,3-dione (see WO 2017197056 for the synthetic method) (58 mg, 0.21 mmol) were added. The resulting mixture was warmed to 85° C. and reacted for 3 h. The reaction liquid was cooled to room temperature, and 20 mL of water was added to precipitate out a large number of yellow solids. The yellow solids were filtered by suction, dissolved in 50 mL of mixed solvent (dichloromethane/methanol (v/v)=10:1), dried over anhydrous sodium sulfate, and concentrated under reduced pressure. The crude product was then separated and purified by silica gel column chromatography (dichloromethane/methanol (v/v)=1:0-9:1), to afford compound 83 (70 mg, yield: 59%).

¹H NMR (400 MHz, DMSO-d₆) δ 11.04 (s, 1H), 8.32 (d, 1H), 7.86-7.82 (m, 1H), 7.68-7.61 (m, 2H), 7.54-7.50 (m, 1H), 7.20-7.12 (m, 2H), 6.98-6.75 (m, 4H), 6.64 (d, 1H), 5.10-4.92 (m, 3H), 3.78-3.66 (m, 4H), 3.60-3.35 (m, 3H), 3.18-3.08 (m, 1H), 2.95-2.80 (m, 1H), 2.69-2.51 (m, 3H), 2.48-2.28 (m, 6H), 2.20-1.91 (m, 2H), 1.81-1.70 (m, 1H), 1.64 (s, 6H), 1.56-1.35 (m, 8H).

LCMS m/z=855.4 [M+1]⁺

### Example 84

### 3-chloro-5-(2-(4-[(2-(9-[(1-(2-(2,6-dioxopiperidin-3-yl)-1,3-dioxo-2,3-dihydro-1H-isoindol-4-yl)piperidin-4-yl)methyl]-3,9-diazaspiro[5.5]undecan-3-yl)pyrimidin-4-yl)methoxy]phenyl)propan-2-yl)benzonitrile (Compound 84)

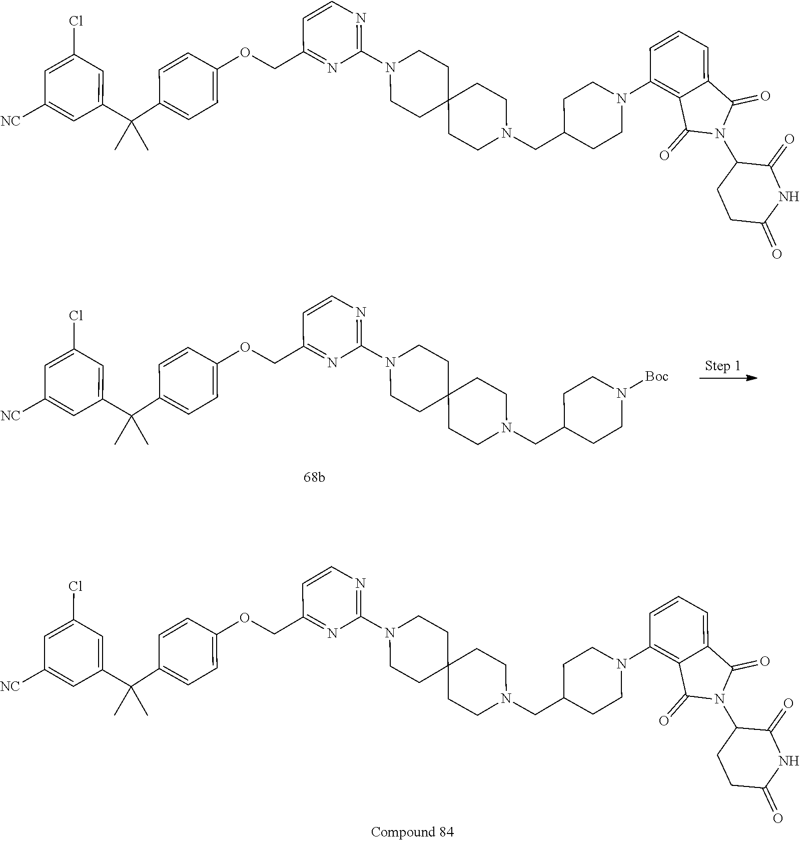 [SMILES: CC(C)(c1ccc(OCc2ccnc(N3CCC4(CCN(CC5CCN(c6cccc7c6C(=O)N(C6CCC(=O)NC6=O)C7=O)CC5)CC4)CC3)n2)cc1)c1cc(Cl)cc(C#N)c1 ; CC(C)(C)OC(=O)N1CCC(CN2CCC3(CC2)CCN(c2nccc(COc4ccc(C(C)(C)c5cc(Cl)cc(C#N)c5)cc4)n2)CC3)CC1 ; CC(C)(c1ccc(OCc2ccnc(N3CCC4(CCN(CC5CCN(c6cccc7c6C(=O)N(C6CCC(=O)NC6=O)C7=O)CC5)CC4)CC3)n2)cc1)c1cc(Cl)cc(C#N)c1]

68b (380 mg, 0.53 mmol) was dissolved in 6 mL of DCM, and 2 mL of trifluoroacetic acid was added. The mixture was stirred at room temperature for 2 h and concentrated under reduced pressure to afford an oil. The oil was dissolved in 10 mL of DMSO, and solid sodium bicarbonate (180 mg, 2.14 mmol) was added. The mixture was stirred at room temperature for 0.5 h, and then 0.45 mL of DIPEA and 2-(2,6-dioxopiperidin-3-yl)-4-fluoroisoindoline-1,3-dione (see WO 2017197056 for the synthetic method) (150 mg, 0.54 mmol) were added. The resulting mixture was warmed to 85° C. and reacted for 3 h. The reaction liquid was cooled to room temperature, and 20 mL of water was added to precipitate out a large number of yellow solids. The yellow solids were filtered by suction, dissolved in 50 mL of mixed solvent (dichloromethane/methanol (v/v)=10:1), dried over anhydrous sodium sulfate, and concentrated under reduced pressure. The crude product was then separated and purified by silica gel column chromatography (dichloromethane/methanol (v/v)=1:0-9:1), to afford compound 84 (77 mg, yield: 17%).

¹H NMR (400 MHz, DMSO-d₆) δ 11.06 (s, 1H), 8.33 (d, 1H), 7.88-7.80 (m, 1H), 7.73-7.60 (m, 2H), 7.55-7.47 (m, 1H), 7.38-7.25 (m, 2H), 7.22-7.10 (m, 2H), 7.00-6.87 (m, 2H), 6.64 (d, 1H), 5.08 (dd, 1H), 4.96 (s, 2H), 3.82-3.60 (m, 6H), 2.97-2.77 (m, 3H), 2.72-2.51 (m, 2H), 2.44-1.95 (m, 7H), 1.90-1.67 (m, 3H), 1.64 (s, 6H), 1.57-1.27 (m, 10H).

LCMS m/z=869.3 [M+1]⁺.

### Example 85

### 3-chloro-5-(2-(4-((2-(4-((1′-(1-(2,6-dioxopiperidin-3-yl)-3-methyl-2-oxo-2,3-dihydro-1H-benzo[d]imidazol-4-yl)-[4,4′-bipiperidin]-1-yl)methyl)piperidin-1-yl)pyrimidin-4-yl)methoxy)phenyl)propan-2-yl)benzonitrile (Compound 85) trifluoroacetate

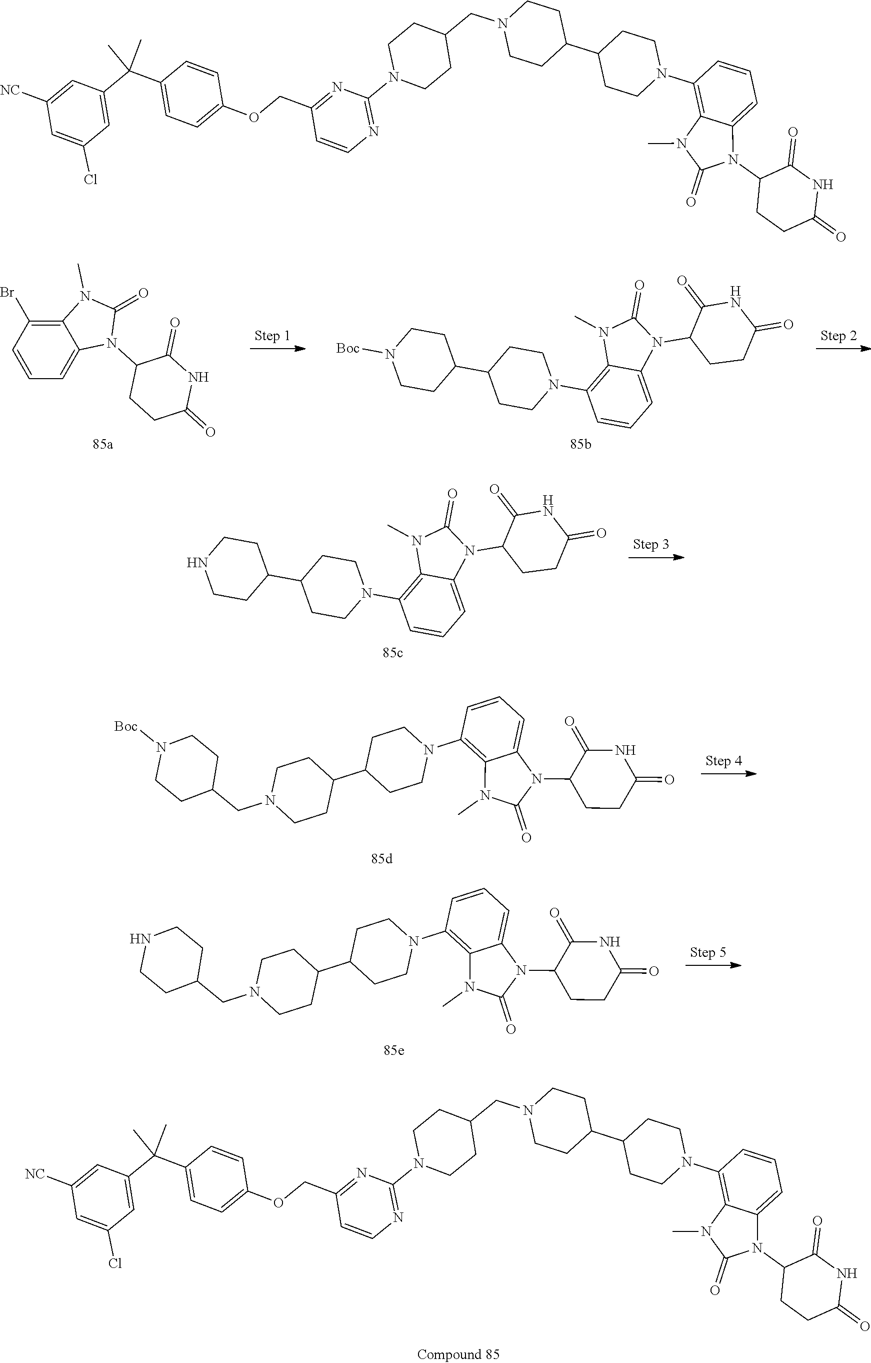

### Step 1: tert-butyl 1′-(1-(2,6-dioxopiperidin-3-yl)-3-methyl-2-oxo-2,3-dihydro-1H-benzo[d]imidazol-4-yl)-[4,4′-bipiperidine]-1-carboxylate (85b)

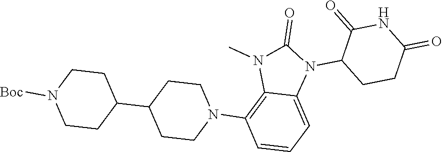 [SMILES: Cn1c(=O)n(C2CCC(=O)NC2=O)c2cccc(N3CCC(C4CCN(C(=O)OC(C)(C)C)CC4)CC3)c21]

85a (see WO 2020113233 for the synthetic method) (700 mg, 2.07 mmol) and tert-butyl [4,4′-bipiperidine]-1-carboxylate (832 mg, 3.10 mmol) were added to 20 mL of toluene, and the mixture was subjected to nitrogen replacement 3 times. RuPhos-Pd-G3 (CAS: 1445085-77-7) (170 mg, 0.20 mmol) was added, and the mixture was subjected to nitrogen replacement 3 times. 1 mol/L solution of LiHMDS in tetrahydrofuran (8.3 mL, 8.3 mmol) was added, and the mixture was subjected to nitrogen replacement 3 times, warmed to 80° C. and reacted for 2 h. The reaction liquid was cooled to room temperature, and 40 mL of saturated aqueous ammonium chloride solution was slowly added to quench the reaction. The mixture was extracted three times with 50 mL of DCM, and the organic phase was washed with 50 mL of water, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water. Gradient elution method: gradient elution with acetonitrile from 5% to 60% (elution time: 15 min), lyophilization was performed to obtain 85b (45 mg, yield: 4%).

LCMS m/z=526.2 [M+1]⁺

### Step 2: 3-(4-([4,4′-bipiperidin]-1-yl)-3-methyl-2-oxo-2,3-dihydro-1H-benzo[d]imidazol-1-yl)piperidine-2,6-dione (85c) trifluoroacetate

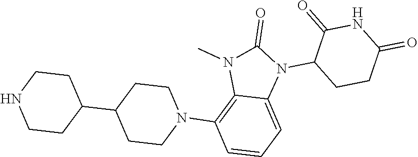 [SMILES: Cn1c(=O)n(C2CCC(=O)NC2=O)c2cccc(N3CCC(C4CCNCC4)CC3)c21]

85b (45 mg, 0.086 mmol) was dissolved in 10 mL of DCM, and 4 mL of trifluoroacetic acid was added. The mixture was stirred at room temperature for 2 h. The reaction liquid was concentrated under reduced pressure, to afford crude 85c trifluoroacetate (60 mg).

LCMS m/z=426.2 [M+1]⁺

### Step 3: tert-butyl 4-((1′-(1-(2,6-dioxopiperidin-3-yl)-3-methyl-2-oxo-2,3-dihydro-1H-benzo[d]imidazol-4-yl)-[4,4′-bipiperidin]-1-yl)methyl)piperidine-1-carboxylate (85d)

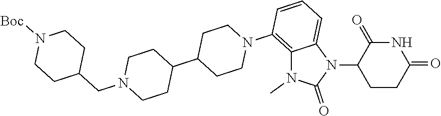 [SMILES: Cn1c(=O)n(C2CCC(=O)NC2=O)c2cccc(N3CCC(C4CCN(CC5CCN(C(=O)OC(C)(C)C)CC5)CC4)CC3)c21]

The above crude 85c trifluoroacetate (60 mg) was dissolved in 15 mL of THF, and solid sodium bicarbonate (22 mg, 0.26 mmol) was added. The mixture was stirred at room temperature for 20 min, and then tert-butyl 4-formylpiperidine-1-carboxylate (37 mg, 0.17 mmol) and 0.1 mL of acetic acid were added. The mixture was stirred at room temperature for 60 min, and then sodium triacetoxyborohydride (37 mg, 0.17 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 85d (50 mg, two-step yield from compound 85b: 93%).

LCMS m/z=623.5 [M+1]⁺

### Step 4: 3-(3-methyl-2-oxo-4-(1′-(piperidin-4-ylmethyl)-[4,4′-bipiperidin]-1-yl)-2,3-dihydro-1H-benzo[d]imidazol-1-yl)piperidine-2,6-dione (85e) trifluoroacetate

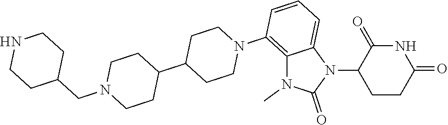 [SMILES: Cn1c(=O)n(C2CCC(=O)NC2=O)c2cccc(N3CCC(C4CCN(CC5CCNCC5)CC4)CC3)c21]

85d) (50 mg, 0.08 mmol) was dissolved in 10 mL of DCM, and 4 mL of trifluoroacetic acid was added. The mixture was stirred at room temperature for 2 h. The reaction liquid was concentrated under reduced pressure, to afford crude 85e trifluoroacetate (65 mg).

LCMS m/z=523.3 [M+1]⁺

### Step 5: 3-chloro-5-(2-(4-((2-(4-((1′-(1-(2,6-dioxopiperidin-3-yl)-3-methyl-2-oxo-2,3-dihydro-1H-benzo[d]imidazol-4-yl)-[4,4′-bipiperidin]-1-yl)methyl)piperidin-1-yl)pyrimidin-4-yl)methoxy)phenyl)propan-2-yl)benzonitrile (Compound 85) trifluoroacetate

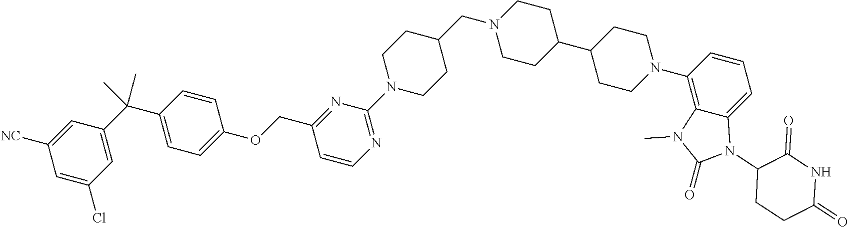 [SMILES: Cn1c(=O)n(C2CCC(=O)NC2=O)c2cccc(N3CCC(C4CCN(CC5CCN(c6nccc(COc7ccc(C(C)(C)c8cc(Cl)cc(C#N)c8)cc7)n6)CC5)CC4)CC3)c21]

3-chloro-5-(2-(4-((2-chloropyrimidin-4-yl)methoxy)phenyl)propan-2-yl)benzonitrile (9d) (48 mg, 0.12 mmol) was dissolved in 10 mL of DMF, and the above crude 85e trifluoroacetate (65 mg) and solid potassium bicarbonate (40 mg, 0.40 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 85 trifluoroacetate (12 mg).

¹H NMR (400 MHz, DMSO-d₆) δ 11.06 (s, 1H), 8.34 (d, 1H), 7.90-7.75 (m, 1H), 7.70-7.45 (m, 2H), 7.30-7.08 (m, 2H), 7.05-6.80 (m, 5H), 6.67 (d, 1H), 5.45-5.23 (m, 1H), 4.96 (s, 2H), 4.75-4.55 (m, 2H), 3.70-3.45 (m, 5H), 3.22-3.04 (m, 3H), 3.04-2.79 (m, 6H), 2.79-2.51 (m, 4H), 2.25-1.85 (m, 4H), 1.85-1.70 (m, 4H), 1.63 (s, 6H), 1.58-1.31 (m, 5H), 1.31-1.03 (m, 3H).

LCMS m/z=884.3 [M+1]⁺

### Example 86

### 3-chloro-5-(2-(4-((2-(3-((9-(2-(2,6-dioxopiperidin-3-yl)-1,3-dioxoisoindolin-5-yl)-3,9-diazaspiro[5.5]undecan-3-yl)methyl)pyrrolidin-1-yl)pyrimidin-4-yl)methoxy)phenyl) propan-2-yl)benzonitrile (Compound 86) trifluoroacetate

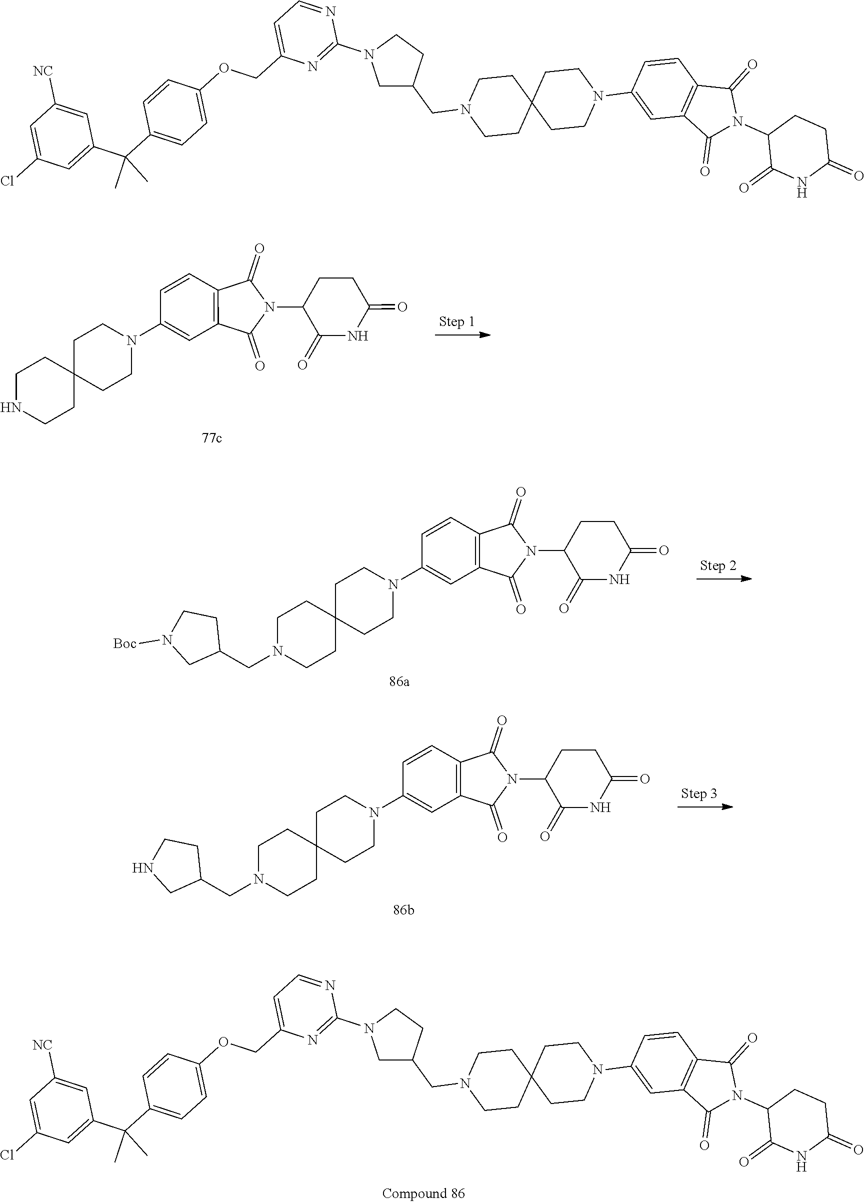 [SMILES: CC(C)(c1ccc(OCc2ccnc(N3CCC(CN4CCC5(CC4)CCN(c4ccc6c(c4)C(=O)N(C4CCC(=O)NC4=O)C6=O)CC5)C3)n2)cc1)c1cc(Cl)cc(C#N)c1 ; O=C1CCC(N2C(=O)c3ccc(N4CCC5(CCNCC5)CC4)cc3C2=O)C(=O)N1 ; CC(C)(C)OC(=O)N1CCC(CN2CCC3(CC2)CCN(c2ccc4c(c2)C(=O)N(C2CCC(=O)NC2=O)C4=O)CC3)C1 ; O=C1CCC(N2C(=O)c3ccc(N4CCC5(CCN(CC6CCNC6)CC5)CC4)cc3C2=O)C(=O)N1 ; CC(C)(c1ccc(OCc2ccnc(N3CCC(CN4CCC5(CC4)CCN(c4ccc6c(c4)C(=O)N(C4CCC(=O)NC4=O)C6=O)CC5)C3)n2)cc1)c1cc(Cl)cc(C#N)c1]

### Step 1: tert-butyl 3-((9-(2-(2,6-dioxopiperidin-3-yl)-1,3-dioxoisoindolin-5-yl)-3,9-diazaspiro[5.5]undecan-3-yl)methyl)pyrrolidine-1-carboxylate (86a)

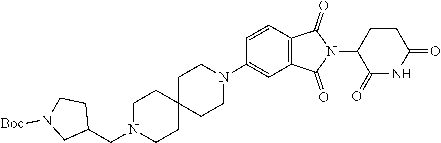 [SMILES: CC(C)(C)OC(=O)N1CCC(CN2CCC3(CC2)CCN(c2ccc4c(c2)C(=O)N(C2CCC(=O)NC2=O)C4=O)CC3)C1]

The above crude 2-(2,6-dioxopiperidin-3-yl)-5-(3,9-diazaspiro[5.5]undecan-3-yl)isoindoline-1,3-dione (77c) trifluoroacetate (290 mg) was dissolved in 15 mL of THF, and solid sodium bicarbonate (123 mg, 1.46 mmol) was added. The mixture was stirred at room temperature for 20 min, and then tert-butyl 3-formylpyrrolidine-1-carboxylate (195 mg, 0.98 mmol) and 0.1 mL of acetic acid were successively added. The mixture was stirred at room temperature for 60 min, and then sodium triacetoxyborohydride (210 mg, 0.99 mmol) was added. The resulting mixture was reacted at room temperature for 16 h. To the reaction system was slowly added 20 mL of saturated aqueous sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=15:1), to afford 86a (280 mg, two-step yield from compound 77b: 96%).

LCMS m/z=594.4 [M+1]⁺

### Step 2: 2-(2,6-dioxopiperidin-3-yl)-5-(9-(pyrrolidin-3-ylmethyl)-3,9-diazaspiro[5.5]undecan-3-yl)isoindoline-1,3-dione (86b) trifluoroacetate

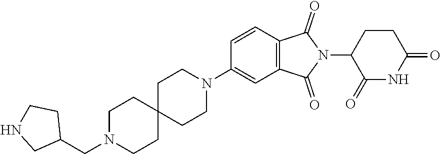 [SMILES: O=C1CCC(N2C(=O)c3ccc(N4CCC5(CCN(CC6CCNC6)CC5)CC4)cc3C2=O)C(=O)N1]

86a (150 mg, 0.25 mmol) was dissolved in 10 mL of DCM, and 4 mL of trifluoroacetic acid was added. The mixture was stirred at room temperature for 2 h. The reaction liquid was concentrated under reduced pressure, to afford crude 86b trifluoroacetate (175 mg).

LCMS m/z=494.3 [M+1]⁺

### Step 3: 3-chloro-5-(2-(4-((2-(3-((9-(2-(2,6-dioxopiperidin-3-yl)-1,3-dioxoisoindolin-5-yl)-3,9-diazaspiro[5.5]undecan-3-yl)methyl)pyrrolidin-1-yl)pyrimidin-4-yl)methoxy)phenyl) propan-2-yl)benzonitrile (Compound 86) trifluoroacetate

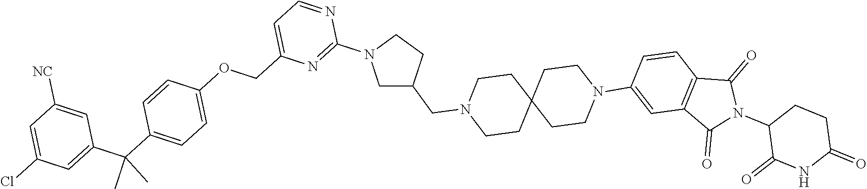 [SMILES: CC(C)(c1ccc(OCc2ccnc(N3CCC(CN4CCC5(CC4)CCN(c4ccc6c(c4)C(=O)N(C4CCC(=O)NC4=O)C6=O)CC5)C3)n2)cc1)c1cc(Cl)cc(C#N)c1]

3-chloro-5-(2-(4-((2-chloropyrimidin-4-yl)methoxy)phenyl)propan-2-yl)benzonitrile (9d) (100 mg, 0.26 mmol) was dissolved in 20 ml of DMF, and the above crude 86b trifluoroacetate (175 mg) and solid potassium bicarbonate (125 mg, 1.25 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 86 trifluoroacetate (45 mg).

¹H NMR (400 MHz, DMSO-d₆) δ 11.06 (s, 1H), 8.36 (d, 1H), 7.87-7.82 (m, 1H), 7.72-7.62 (m, 2H), 7.56-7.50 (m, 1H), 7.38-7.32 (m, 1H), 7.26 (dd, 1H), 7.21-7.12 (m, 2H), 6.98-6.90 (m, 2H), 6.72 (d, 1H), 5.13-4.93 (m, 3H), 3.93-3.82 (m, 1H), 3.75-3.66 (m, 1H), 3.56-3.36 (m, 7H), 3.35-3.00 (m, 5H), 2.97-2.70 (m, 2H), 2.69-2.51 (m, 2H), 2.26-2.12 (m, 1H), 2.08-1.85 (m, 3H), 1.83-1.42 (m, 13H).

LCMS m/z=855.3 [M+1]⁺

### Example 87

### 3-chloro-5-(2-(4-((2-(3-((9-(2-(2,6-dioxopiperidin-3-yl)-1,3-dioxoisoindolin-5-yl)-3,9-diazaspiro[5.5]undecan-3-yl)methyl)azetidin-1-yl)pyrimidin-4-yl)methoxy)phenyl)propan-2-yl)benzonitrile (Compound 87) trifluoroacetate

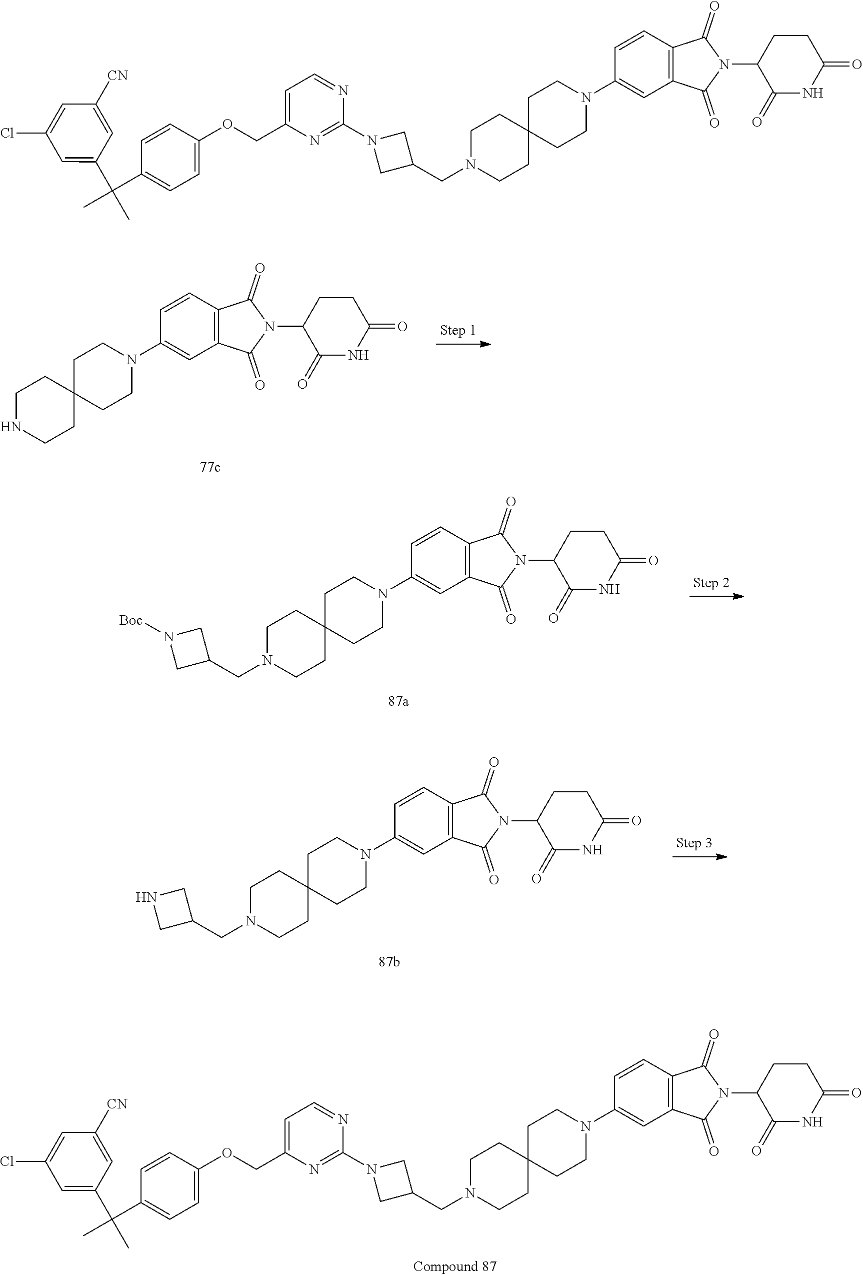 [SMILES: CC(C)(c1ccc(OCc2ccnc(N3CC(CN4CCC5(CC4)CCN(c4ccc6c(c4)C(=O)N(C4CCC(=O)NC4=O)C6=O)CC5)C3)n2)cc1)c1cc(Cl)cc(C#N)c1 ; O=C1CCC(N2C(=O)c3ccc(N4CCC5(CCNCC5)CC4)cc3C2=O)C(=O)N1 ; CC(C)(C)OC(=O)N1CC(CN2CCC3(CC2)CCN(c2ccc4c(c2)C(=O)N(C2CCC(=O)NC2=O)C4=O)CC3)C1 ; O=C1CCC(N2C(=O)c3ccc(N4CCC5(CCN(CC6CNC6)CC5)CC4)cc3C2=O)C(=O)N1 ; CC(C)(c1ccc(OCc2ccnc(N3CC(CN4CCC5(CC4)CCN(c4ccc6c(c4)C(=O)N(C4CCC(=O)NC4=O)C6=O)CC5)C3)n2)cc1)c1cc(Cl)cc(C#N)c1]

### Step 1: tert-butyl 3-((9-(2-(2,6-dioxopiperidin-3-yl)-1,3-dioxoisoindolin-5-yl)-3,9-diazaspiro[0.5]undecan-3-yl)methyl)azetidine-1-carboxylate (87a)

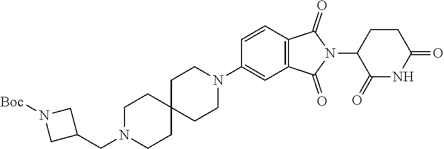 [SMILES: CC(C)(C)OC(=O)N1CC(CN2CCC3(CC2)CCN(c2ccc4c(c2)C(=O)N(C2CCC(=O)NC2=O)C4=O)CC3)C1]

The above crude 77c trifluoroacetate (290 mg) was dissolved in 15 mL of THF, and solid sodium bicarbonate (123 mg, 1.46 mmol) was added. The mixture was stirred at room temperature for 20 min, and then tert-butyl 3-formylazetidine-1-carboxylate (210 mg, 1.14 mmol) and 0.1 mL of acetic acid were successively added. The mixture was stirred at room temperature for 60 min, and then sodium triacetoxyborohydride (210 mg, 0.99 mmol) was added. The resulting mixture was reacted at room temperature for 16 h. To the reaction system was slowly added 20 mL of saturated aqueous sodium bicarbonate solution, and the mixture was extracted with 80 mL of DCM. The organic phase was washed with 50 mL of water, dried over anhydrous sodium sulfate, and concentrated under reduced pressure. The crude product was separated and purified by silica gel column chromatography (dichloromethane/methanol (v/v)=15:1), to afford 87a (260 mg, two-step yield from compound 77b: 92%).

LCMS m/z=580.3 [M+1]⁺

### Step 2: 5-(9-(azetidin-3-ylmethyl)-3,9-diazaspiro[5.5]undecan-3-yl)-2-(2,6-dioxopiperidin-3-yl)isoindoline-1,3-dione (87b) trifluoroacetate

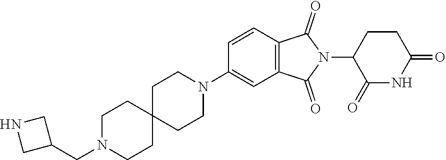 [SMILES: O=C1CCC(N2C(=O)c3ccc(N4CCC5(CCN(CC6CNC6)CC5)CC4)cc3C2=O)C(=O)N1]

87a (150 mg, 0.26 mmol) was dissolved in 10 mL of DCM, and 4 mL of trifluoroacetic acid was added. The mixture was stirred at room temperature for 2 h. The reaction liquid was concentrated under reduced pressure, to afford crude 87b trifluoroacetate (175 mg).

LCMS m/z=480.3 [M+1]⁺

### Step 3: 3-chloro-5-(2-(4-((2-(3-((9-(2-(2,6-dioxopiperidin-3-yl)-1,3-dioxoisoindolin-5-yl)-3,9-diazaspiro[5.5]undecan-3-yl)methyl)azetidin-1-yl)pyrimidin-4-yl)methoxy)phenyl) propan-2-yl)benzonitrile (Compound 87) trifluoroacetate

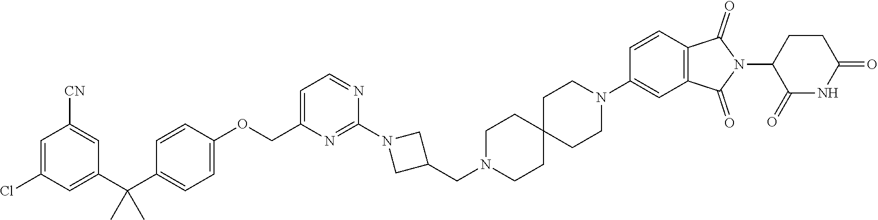 [SMILES: CC(C)(c1ccc(OCc2ccnc(N3CC(CN4CCC5(CC4)CCN(c4ccc6c(c4)C(=O)N(C4CCC(=O)NC4=O)C6=O)CC5)C3)n2)cc1)c1cc(Cl)cc(C#N)c1]

3-chloro-5-(2-(4-((2-chloropyrimidin-4-yl)methoxy)phenyl)propan-2-yl)benzonitrile (9d) (100 mg, 0.26 mmol) was dissolved in 20 ml of DMF, and the above crude 5-(9-(azetidin-3-ylmethyl)-3,9-diazaspiro[5.5]undecan-3-yl)-2-(2,6-dioxopiperidin-3-yl)isoindoline-1,3-dione (87b) trifluoroacetate (175 mg) and solid potassium bicarbonate (125 mg, 1.25 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 87 trifluoroacetate (30 mg).

¹H NMR (400 MHz, DMSO-d₆) δ 11.06 (s, 1H), 8.38 (d, 1H), 7.98-7.80 (m, 1H), 7.72-7.62 (m, 2H), 7.56-7.50 (m, 1H), 7.38-7.31 (m, 1H), 7.30-7.22 (m, 1H), 7.21-7.14 (m, 2H), 6.98-6.90 (m, 2H), 6.80 (d, 1H), 5.07 (dd, 1H), 4.98 (s, 2H), 4.30-4.16 (m, 2H), 3.96-3.80 (m, 2H), 3.66-3.48 (m, 4H), 3.38-3.25 (m, 4H), 3.23-3.00 (m, 3H), 2.97-2.82 (m, 1H), 2.73-2.52 (m, 2H), 2.09-1.84 (m, 3H), 1.80-1.42 (m, 12H).

LCMS m/z=841.3 [M+1]⁺

### Example 88

### 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)benzonitrile (Compound 88) trifluoroacetate

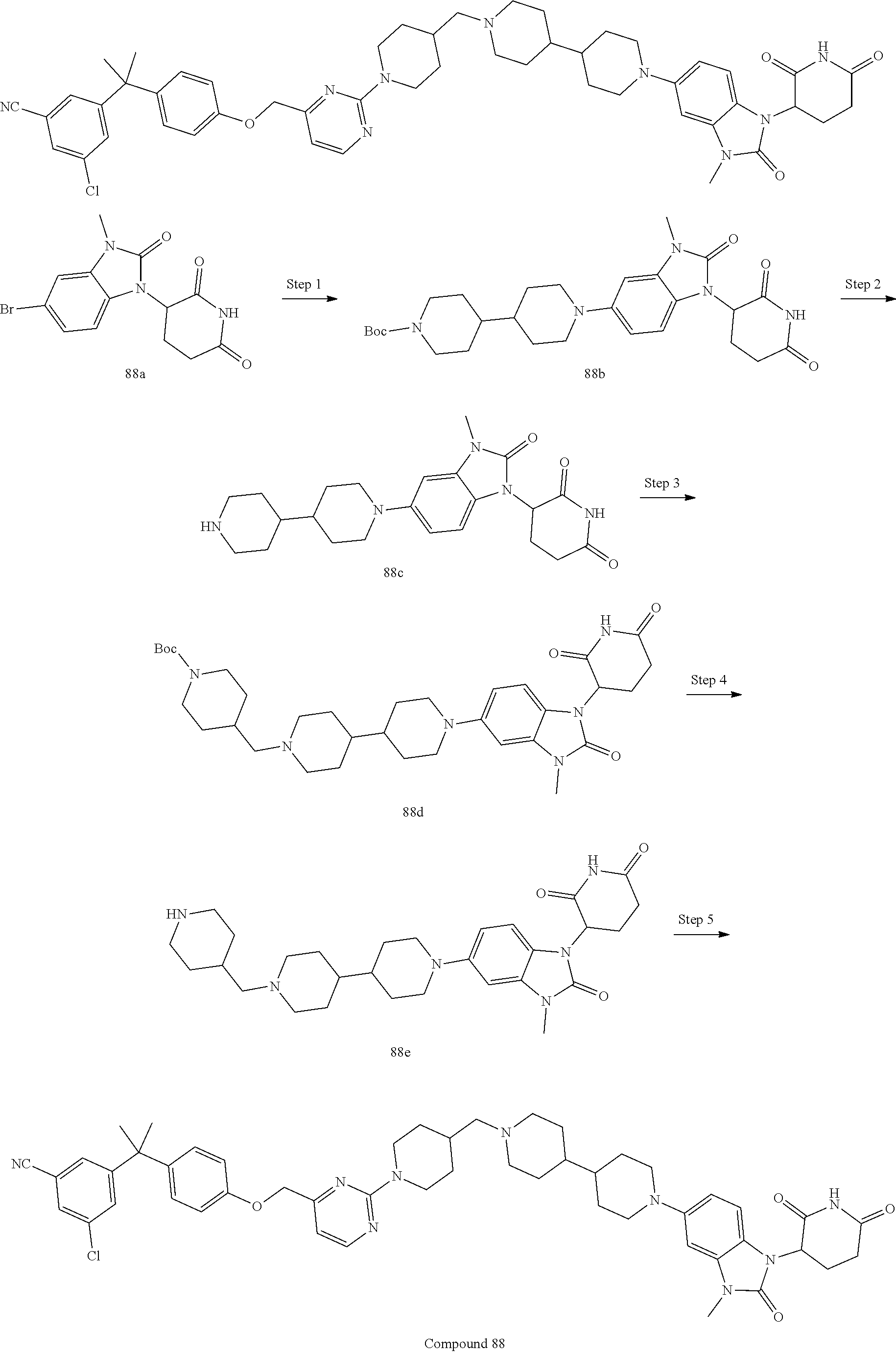

### Step 1: tert-butyl 1′-(1-(2,6-dioxopiperidin-3-yl)-3-methyl-2-oxo-2,3-dihydro-1H-benzo[d]imidazol-5-yl)-[4,4′-bipiperidine]-1-carboxylate (88b)

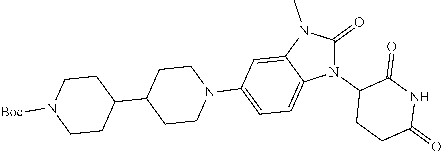 [SMILES: Cn1c(=O)n(C2CCC(=O)NC2=O)c2ccc(N3CCC(C4CCN(C(=O)OC(C)(C)C)CC4)CC3)cc21]

88a (see WO 2020113233 for the synthetic method) (1.0 g, 2.96 mmol) and tert-butyl [4,4′-bipiperidine]-1-carboxylate (1.20 g, 4.47 mmol) were added to 35 mL of toluene, and the mixture was subjected to nitrogen replacement 3 times. RuPhos-Pd-G3 (CAS: 1445085-77-7) (250 mg, 0.29 mmol) and RuPhos (140 mg, 0.30 mmol) were added, and the mixture was subjected to nitrogen replacement 3 times. 1 mol/L solution of LiHMDS in tetrahydrofuran (12 mL, 12.0 mmol) was added, and the mixture was subjected to nitrogen replacement 3 times, warmed to 80° C. and reacted for 2 h. The reaction liquid was cooled to room temperature, and 40 mL of saturated aqueous ammonium chloride solution was slowly added. The mixture was extracted three times with 50 mL of DCM, and the organic phase was washed with 50 mL of water, dried over anhydrous sodium sulfate, and concentrated under reduced pressure. The crude product was separated and purified by silica gel column chromatography (ethyl acetate/petroleum ether (v/v)=0:1-1:0), to afford 88b (650 mg, yield: 42%).

LCMS m/z=526.2 [M+1]⁺

### Step 2: 3-(5-([4,4′-bipiperidin]-1-yl)-3-methyl-2-oxo-2,3-dihydro-1H-benzo[d]imidazol-1-yl)piperidine-2,6-dione (88c) trifluoroacetate

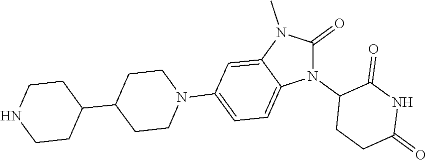 [SMILES: Cn1c(=O)n(C2CCC(=O)NC2=O)c2ccc(N3CCC(C4CCNCC4)CC3)cc21]

88b (200 mg, 0.38 mmol) was dissolved in 10 mL of DCM, and 4 mL of trifluoroacetic acid was added. The mixture was stirred at room temperature for 2 h. The reaction liquid was concentrated under reduced pressure, to afford crude 88c trifluoroacetate (240 mg).

LCMS m/z=426.2 [M+1]⁺

### Step 3: tert-butyl 4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidine-1-carboxylate (88d)

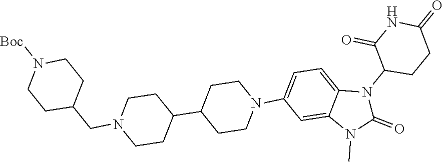 [SMILES: Cn1c(=O)n(C2CCC(=O)NC2=O)c2ccc(N3CCC(C4CCN(CC5CCN(C(=O)OC(C)(C)C)CC5)CC4)CC3)cc21]

The above crude 88c trifluoroacetate (240 mg) was dissolved in 15 mL of THF, and solid sodium bicarbonate (85 mg, 1.01 mmol) was added. The mixture was stirred at room temperature for 20 min, and then tert-butyl 4-formylpiperidine-1-carboxylate (138 mg, 0.65 mmol) and 0.1 mL of acetic acid were added. The mixture was stirred at room temperature for 60 min, and then sodium triacetoxyborohydride (137 mg, 0.65 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 88d (220 mg, two-step yield from compound 88b: 93%).

LCMS m/z=623.5 [M+1]⁺

### Step 4: 3-(3-methyl-2-oxo-5-(1′-(piperidin-4-ylmethyl)-[4,4′-bipiperidin]-1-yl)-2,3-dihydro-1H-benzo[d]imidazol-1-yl)piperidine-2,6-dione (88e) trifluoroacetate

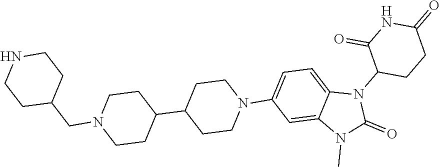 [SMILES: Cn1c(=O)n(C2CCC(=O)NC2=O)c2ccc(N3CCC(C4CCN(CC5CCNCC5)CC4)CC3)cc21]

88d (200 mg, 0.32 mmol) was dissolved in 10 mL of DCM, and 4 mL of trifluoroacetic acid was added. The mixture was stirred at room temperature for 2 h. The reaction liquid was concentrated under reduced pressure, to afford crude 88e trifluoroacetate (230 mg).

LCMS m/z=523.3 [M+1]⁺

### Step 5: 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)benzonitrile (Compound 88) trifluoroacetate

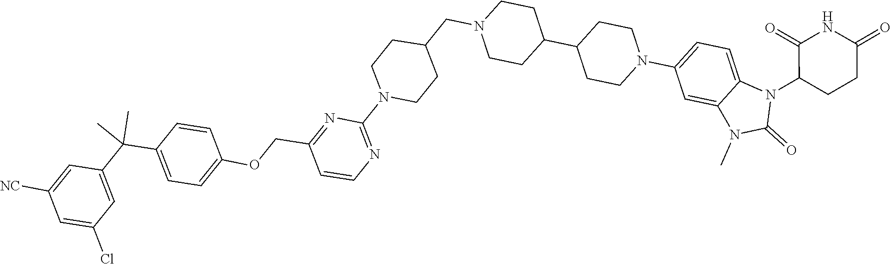 [SMILES: Cn1c(=O)n(C2CCC(=O)NC2=O)c2ccc(N3CCC(C4CCN(CC5CCN(c6nccc(COc7ccc(C(C)(C)c8cc(Cl)cc(C#N)c8)cc7)n6)CC5)CC4)CC3)cc21]

3-chloro-5-(2-(4-((2-chloropyrimidin-4-yl)methoxy)phenyl)propan-2-yl)benzonitrile (9d) (154 mg, 0.38 mmol) was dissolved in 10 ml of DMF, and the above crude 88e trifluoroacetate (230 mg) and solid potassium bicarbonate (256 mg, 2.56 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 88 trifluoroacetate (75 mg).

¹H NMR (400 MHz, DMSO-d₆) δ 11.10 (s, 1H), 8.36 (d, 1H), 7.90-7.80 (m, 1H), 7.70-7.60 (m, 1H), 7.56-7.49 (m, 1H), 7.45-7.04 (m, 5H), 7.04-6.86 (m, 2H), 6.70 (d, 1H), 5.48-5.28 (m, 1H), 4.98 (s, 2H), 4.74-4.58 (m, 2H), 3.75-3.57 (m, 4H), 3.36 (s, 3H), 3.06-2.82 (m, 8H), 2.80-2.56 (m, 3H), 2.23-1.75 (m, 8H), 1.73-1.36 (m, 12H), 1.26-1.06 (m, 2H).

LCMS m/z=884.3 [M+1]⁺

### Example 89

### 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)-4-fluoropiperidin-1-yl)pyrimidin-4-yl)methoxy)phenyl)propan-2-yl)benzonitrile (Compound 89)

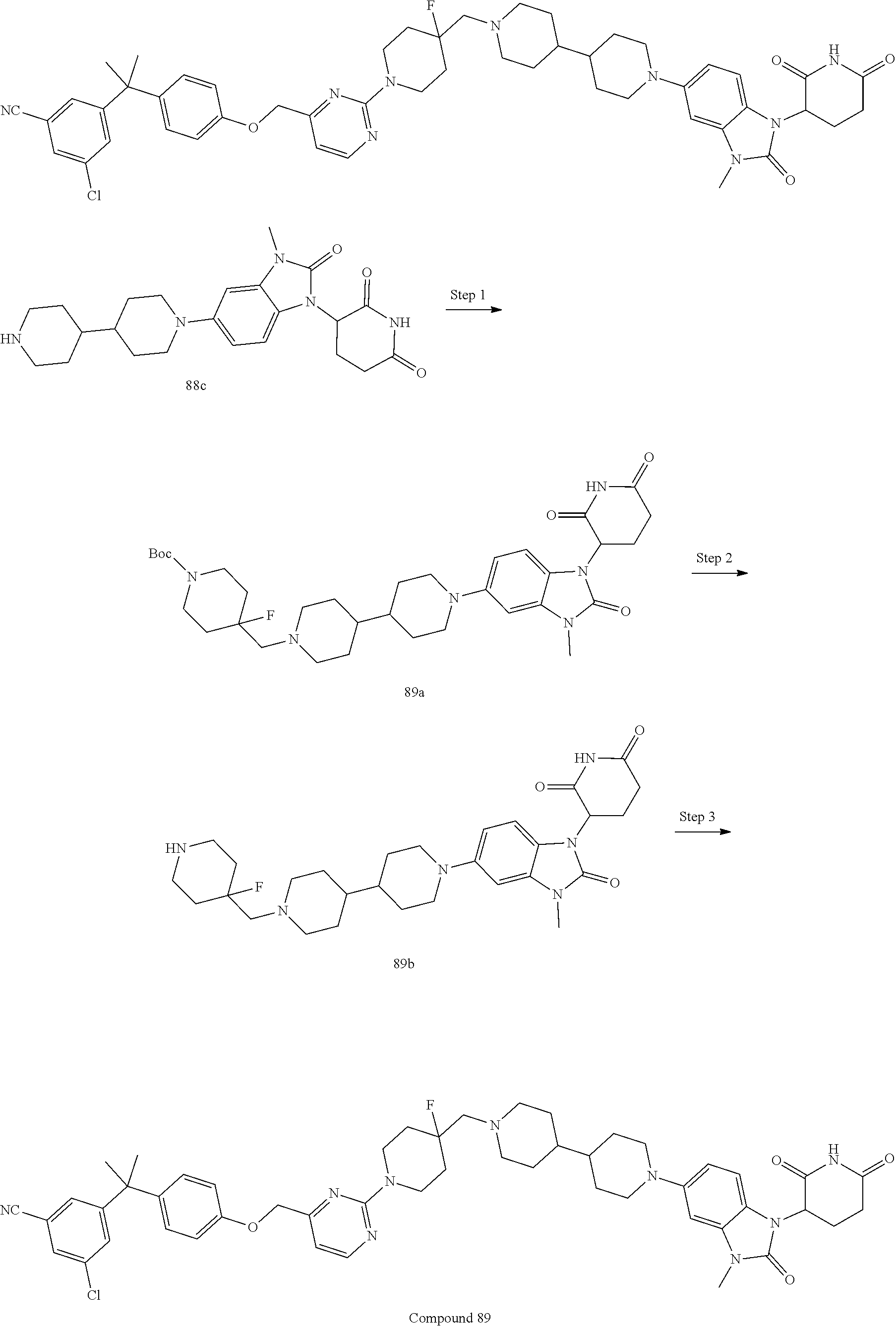 [SMILES: Cn1c(=O)n(C2CCC(=O)NC2=O)c2ccc(N3CCC(C4CCN(CC5(F)CCN(c6nccc(COc7ccc(C(C)(C)c8cc(Cl)cc(C#N)c8)cc7)n6)CC5)CC4)CC3)cc21 ; Cn1c(=O)n(C2CCC(=O)NC2=O)c2ccc(N3CCC(C4CCNCC4)CC3)cc21 ; Cn1c(=O)n(C2CCC(=O)NC2=O)c2ccc(N3CCC(C4CCN(CC5(F)CCN(C(=O)OC(C)(C)C)CC5)CC4)CC3)cc21 ; Cn1c(=O)n(C2CCC(=O)NC2=O)c2ccc(N3CCC(C4CCN(CC5(F)CCNCC5)CC4)CC3)cc21 ; Cn1c(=O)n(C2CCC(=O)NC2=O)c2ccc(N3CCC(C4CCN(CC5(F)CCN(c6nccc(COc7ccc(C(C)(C)c8cc(Cl)cc(C#N)c8)cc7)n6)CC5)CC4)CC3)cc21]

### Step 1: tert-butyl 4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)-4-fluoropiperidine-1-carboxylate (89a)

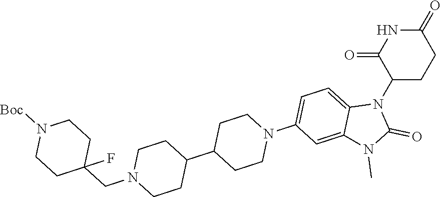 [SMILES: Cn1c(=O)n(C2CCC(=O)NC2=O)c2ccc(N3CCC(C4CCN(CC5(F)CCN(C(=O)OC(C)(C)C)CC5)CC4)CC3)cc21]

The above 88c trifluoroacetate (240 mg) was dissolved in 15 mL of THF, and solid sodium bicarbonate (128 mg, 1.52 mmol) was added. The mixture was stirred at room temperature for 20 min, and then tert-butyl 4-fluoro-4-formylpiperidine-1-carboxylate (see WO 2014063587 for the synthetic method) (176 mg, 0.76 mmol) and 0.1 mL of acetic acid were added. The mixture was stirred at room temperature for 60 min, and then sodium triacetoxyborohydride (161 mg, 0.76 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 89a (110 mg, two-step yield from compound 88b: 45%).

LCMS m/z=641.4 [M+1]⁺

### Step 2: 3-(5-(1′-((4-fluoropiperidin-4-yl)methyl)-[4,4′-bipiperidin]-1-yl)-3-methyl-2-oxo-2,3-dihydro-1H-benzo[d]imidazol-1-yl)piperidine-2,6-dione (89b) trifluoroacetate

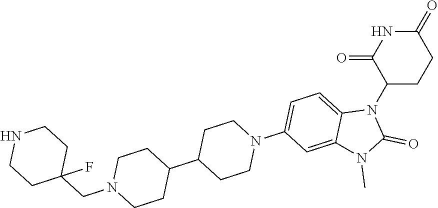 [SMILES: Cn1c(=O)n(C2CCC(=O)NC2=O)c2ccc(N3CCC(C4CCN(CC5(F)CCNCC5)CC4)CC3)cc21]

89a (110 mg, 0.17 mmol) was dissolved in 10 mL of DCM, and 4 mL of trifluoroacetic acid was added. The mixture was stirred at room temperature for 2 h. The reaction liquid was concentrated under reduced pressure, to afford crude 89b trifluoroacetate (140 mg).

LCMS m/z=541.4 [M+1]⁺

### Step 3: 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)-4-fluoropiperidin-1-yl)pyrimidin-4-yl)methoxy)phenyl)propan-2-yl)benzonitrile (Compound 89)

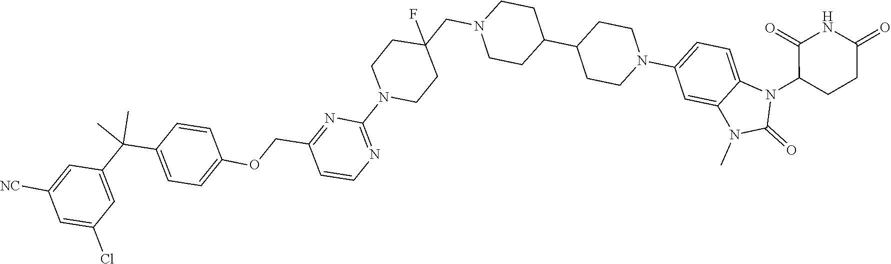 [SMILES: Cn1c(=O)n(C2CCC(=O)NC2=O)c2ccc(N3CCC(C4CCN(CC5(F)CCN(c6nccc(COc7ccc(C(C)(C)c8cc(Cl)cc(C#N)c8)cc7)n6)CC5)CC4)CC3)cc21]

3-chloro-5-(2-(4-((2-chloropyrimidin-4-yl)methoxy)phenyl)propan-2-yl)benzonitrile (9d) (105 mg, 0.26 mmol) was dissolved in 6 ml of DMF, and the above crude 89b trifluoroacetate (140 mg) and solid potassium bicarbonate (136 mg, 1.36 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 89 (25 mg, two-step yield from compound 89a: 16%).

¹H NMR (400 MHz, DMSO-d₆) δ 11.04 (s, 1H), 8.36 (d, 1H), 7.88-7.78 (m, 1H), 7.70-7.60 (m, 1H), 7.56-7.44 (m, 1H), 7.25-7.10 (m, 2H), 7.04-6.86 (m, 3H), 6.85-6.75 (m, 1H), 6.73-6.55 (m, 2H), 5.40-5.20 (m, 1H), 4.99 (s, 2H), 4.48-4.25 (m, 2H), 3.72-3.50 (m, 2H), 3.30 (s, 3H), 3.29-3.18 (m, 2H), 3.00-2.80 (m, 3H), 2.76-2.40 (m, 6H), 2.14-1.50 (m, 17H), 1.40-0.94 (m, 6H).

LCMS m/z=451.9 [M/2+1]⁺

### Example 90

### 3-chloro-5-(2-(4-((2-(4-((4-(1-(2,6-dioxopiperidin-3-yl)-3-methyl-2-oxo-2,3-dihydro-1H-benzo[d]imidazol-5-yl)-[1,4′-bipiperidin]-1′-yl)methyl)-4-fluoropiperidin-1-yl)pyrimidin-4-yl)methoxy)phenyl)propan-2-yl)benzonitrile (Compound 90) trifluoroacetate

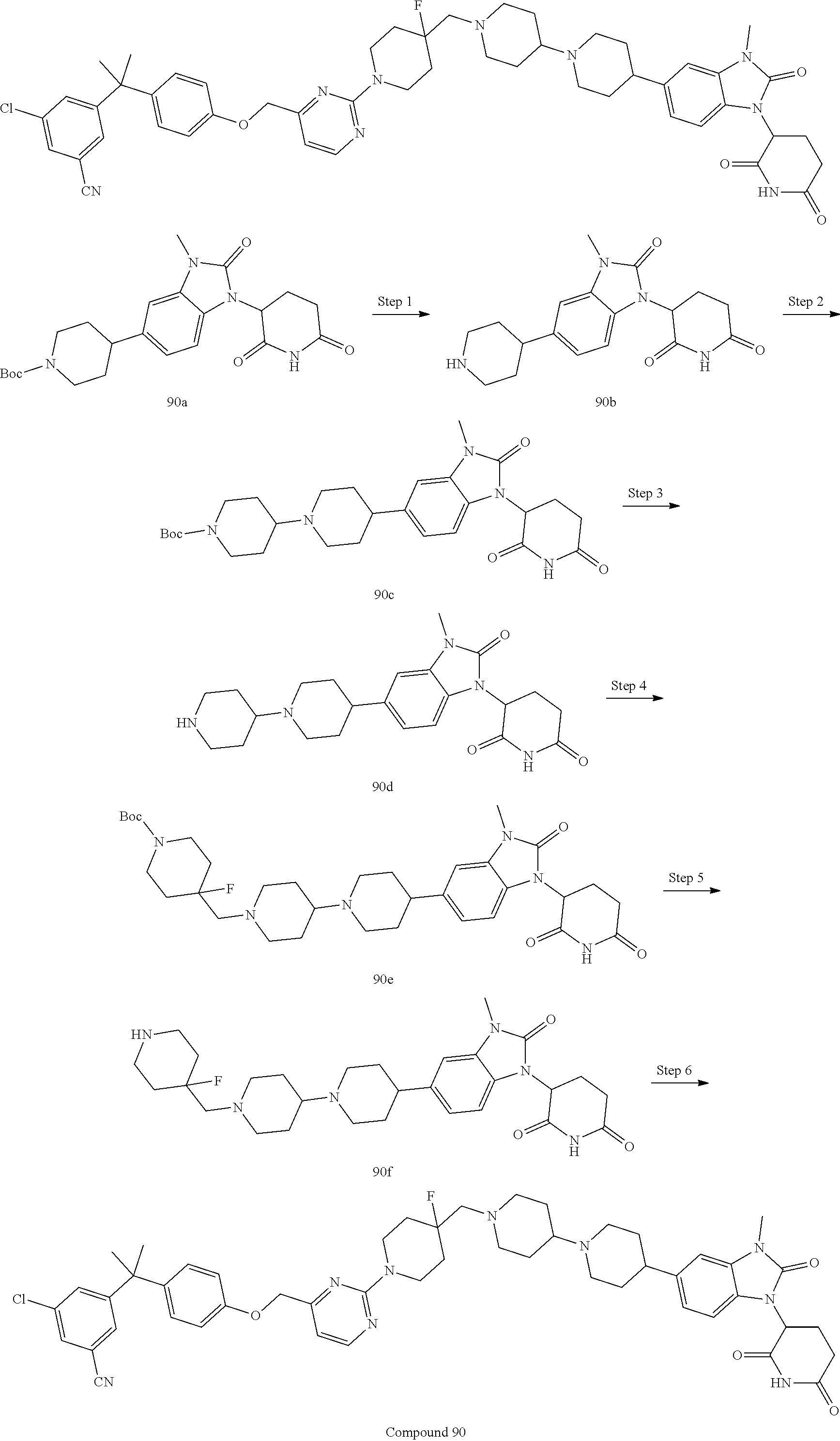

### Step 1: 3-(3-methyl-2-oxo-5-(piperidin-4-yl)-2,3-dihydro-1H-benzo[d]imidazol-1-yl)piperidine-2,6-dione (90b) trifluoroacetate

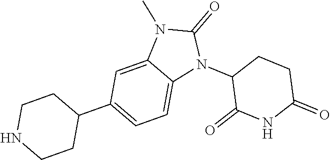 [SMILES: Cn1c(=O)n(C2CCC(=O)NC2=O)c2ccc(C3CCNCC3)cc21]

Tert-butyl 4-(1-(2,6-dioxopiperidin-3-yl)-3-methyl-2-oxo-2,3-dihydro-1H-benzo[d]imidazol-5-yl)piperidine-1-carboxylate (90a) (see WO 2020264499 for the synthetic method) (150 mg, 0.34 mmol) was dissolved in 10 mL of DCM, and 4 mL of trifluoroacetic acid was added. The mixture was stirred at room temperature for 2 h. The reaction liquid was concentrated under reduced pressure, to afford crude 90b trifluoroacetate (180 mg).

LCMS m/z=343.1 [M+1]⁺

### Step 2: tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate (90c)

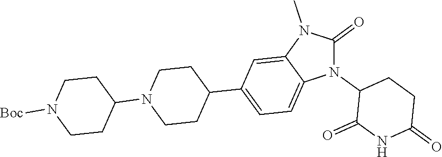 [SMILES: Cn1c(=O)n(C2CCC(=O)NC2=O)c2ccc(C3CCN(C4CCN(C(=O)OC(C)(C)C)CC4)CC3)cc21]

The above crude 90b trifluoroacetate (180 mg) was dissolved in 15 mL of THF, and solid sodium bicarbonate (57 mg, 0.68 mmol) was added. The mixture was stirred at room temperature for 20 min, and then N-Boc-4-piperidone (135 mg, 0.68 mmol) and 0.1 mL of acetic acid were successively added. The mixture was warmed to 50° C. and stirred for 2 h, and then cooled to room temperature. Sodium triacetoxyborohydride (145 mg, 0.68 mmol) was added, and the resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 90c (130 mg, two-step yield from compound 90a: 73%).

LCMS m/z=526.3 [M+1]⁺

### Step 3: 3-(5-([1,4′-bipiperidin]-4-yl)-3-methyl-2-oxo-2,3-dihydro-1H-benzo[d]imidazol-1-yl)piperidine-2,6-dione (90d) trifluoroacetate

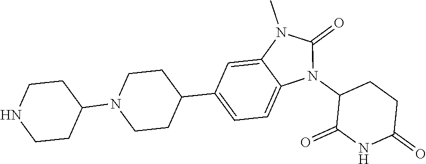 [SMILES: Cn1c(=O)n(C2CCC(=O)NC2=O)c2ccc(C3CCN(C4CCNCC4)CC3)cc21]

90c (120 mg, 0.23 mmol) was dissolved in 10 mL of DCM, and 4 mL of trifluoroacetic acid was added. The mixture was stirred at room temperature for 2 h. The reaction liquid was concentrated under reduced pressure, to afford crude 90d trifluoroacetate (150 mg).

LCMS m/z=426.3 [M+1]⁺

### Step 4: tert-butyl 4-((4-(1-(2,6-dioxopiperidin-3-yl)-3-methyl-2-oxo-2,3-dihydro-1H-benzo[d]imidazol-5-yl)-[1,4′-bipiperidin]-1′-yl)methyl)-4-fluoropiperidine-1-carboxylate (90e)

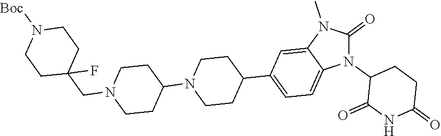 [SMILES: Cn1c(=O)n(C2CCC(=O)NC2=O)c2ccc(C3CCN(C4CCN(CC5(F)CCN(C(=O)OC(C)(C)C)CC5)CC4)CC3)cc21]

The above crude 90d trifluoroacetate (150 mg) was dissolved in 15 mL of THF, and solid sodium bicarbonate (85 mg, 1.01 mmol) was added. The mixture was stirred at room temperature for 20 min, and then tert-butyl 4-fluoro-4-formylpiperidine-1-carboxylate (see WO 2014063587 for the synthetic method) (110 mg, 0.48 mmol) and 0.1 mL of acetic acid were added. The mixture was stirred at room temperature for 2 h, and then sodium triacetoxyborohydride (105 mg, 0.50 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 90e (80 mg, two-step yield from compound 90c: 54%).

LCMS m/z=641.3 [M+1]⁺

### Step 5: 3-(5-(1′-((4-fluoropiperidin-4-yl)methyl)-[1,4′-bipiperidin]-4-yl)-3-methyl-2-oxo-2,3-dihydro-1H-benzo[d]imidazol-1-yl)piperidine-2,6-dione (90f) trifluoroacetate

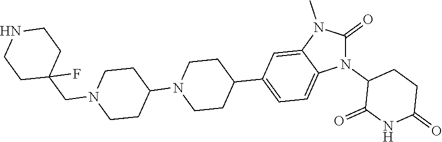 [SMILES: Cn1c(=O)n(C2CCC(=O)NC2=O)c2ccc(C3CCN(C4CCN(CC5(F)CCNCC5)CC4)CC3)cc21]

90e (80 mg, 0.12 mmol) was dissolved in 10 mL of DCM, and 4 mL of trifluoroacetic acid was added. The mixture was stirred at room temperature for 2 h. The reaction liquid was concentrated under reduced pressure, to afford crude 90f trifluoroacetate (100 mg).

LCMS m/z=541.3 [M+1]⁺

### Step 6: 3-chloro-5-(2-(4-((2-(4-((4-(1-(2,6-dioxopiperidin-3-yl)-3-methyl-2-oxo-2,3-dihydro-1H-benzo[d]imidazol-5-yl)-[1,4′-bipiperidin]-1′-yl)methyl)-4-fluoropiperidin-1-yl)pyrimidin-4-yl)methoxy)phenyl)propan-2-yl)benzonitrile (Compound 90) trifluoroacetate

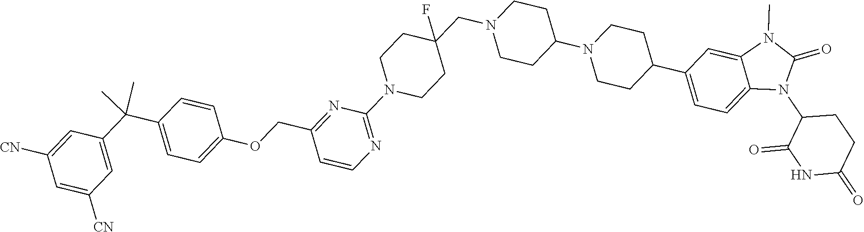

3-chloro-5-(2-(4-((2-chloropyrimidin-4-yl)methoxy)phenyl)propan-2-yl)benzonitrile (9d) (74 mg, 0.18 mmol) was dissolved in 6 ml of DMF, and the above crude 90f trifluoroacetate (100 mg) and solid potassium bicarbonate (100 mg, 1.00 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 90 trifluoroacetate (45 mg).

¹H NMR (400 MHz, DMSO-d₆) δ 11.08 (s, 1H), 8.40 (d, 1H), 7.90-7.80 (m, 1H), 7.70-7.60 (m, 1H), 7.57-7.49 (m, 1H), 7.24-6.85 (m, 7H), 6.75 (d, 1H), 5.42-5.29 (m, 1H), 5.00 (s, 2H), 4.55-4.39 (m, 2H), 3.65-3.50 (m, 2H), 3.35 (s, 3H), 3.34-3.05 (m, 8H), 3.00-2.80 (m, 4H), 2.80-2.56 (m, 3H), 2.30-1.86 (m, 11H), 1.85-1.54 (m, 8H).

LCMS m/z=902.3 [M+1]⁺

### Example 91

### 3-chloro-5-(2-(4-((2-(4-((4-(1-(2,6-dioxopiperidin-3-yl)-3-methyl-2-oxo-2,3-dihydro-1H-benzo[d]imidazol-5-yl)piperidin-1-yl)methyl)-[1,4′-bipiperidin]-1′-yl)pyrimidin-4-yl)methoxy)phenyl)propan-2-yl)benzonitrile (Compound 91) trifluoroacetate

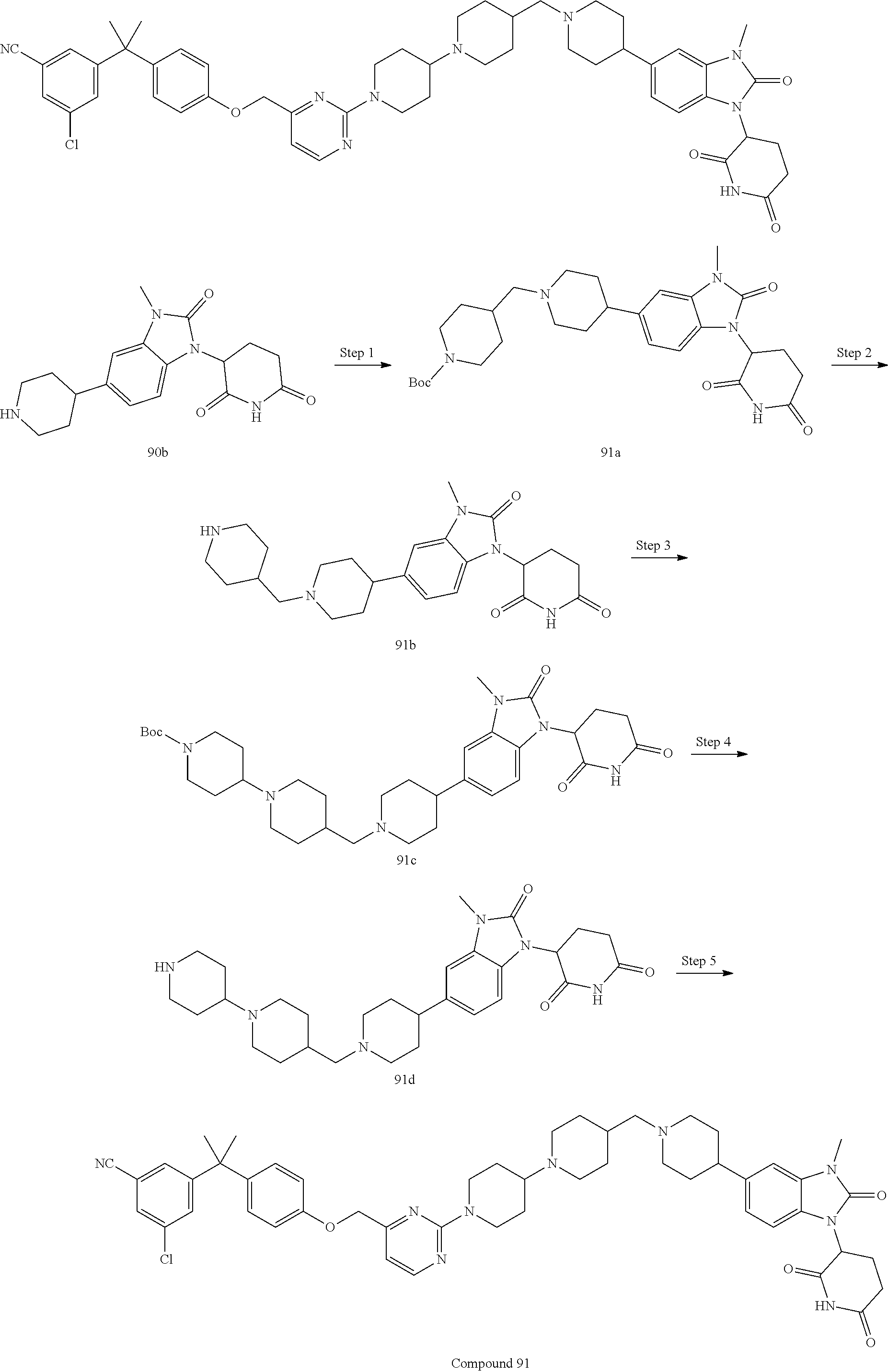

### Step: 1: tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate (91a)

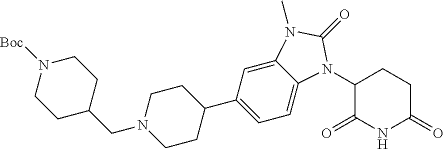 [SMILES: Cn1c(=O)n(C2CCC(=O)NC2=O)c2ccc(C3CCN(CC4CCN(C(=O)OC(C)(C)C)CC4)CC3)cc21]

The above crude 90b trifluoroacetate (150 mg) was dissolved in 15 mL of THF, and solid sodium bicarbonate (45 mg, 0.54 mmol) was added. The mixture was stirred at room temperature for 20 min, and then tert-butyl 4-formylpiperidine-1-carboxylate (116 mg, 0.54 mmol) and 0.1 mL of acetic acid were successively added. The mixture was stirred at room temperature for 60 min, and then sodium triacetoxyborohydride (115 mg, 0.54 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 91a (130 mg, two-step yield from compound 90a: 85%).

LCMS m/z=540.3 [M+1]⁺

### Step 2: 3-(3-methyl-2-oxo-5-(1-(piperidin-4-ylmethyl)piperidin-4-yl)-2,3-dihydro-1H-benzo[d]imidazol-1-yl)piperidine-2,6-dione (91b) trifluoroacetate

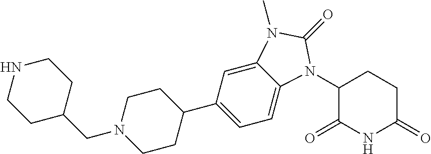 [SMILES: Cn1c(=O)n(C2CCC(=O)NC2=O)c2ccc(C3CCN(CC4CCNCC4)CC3)cc21]

91a (130 mg, 0.24 mmol) was dissolved in 10 mL of DCM, and 4 mL of trifluoroacetic acid was added. The mixture was stirred at room temperature for 2 h. The reaction liquid was concentrated under reduced pressure, to afford crude 91b trifluoroacetate (160 mg).

LCMS m/z=440.3 [M+1]⁺

### Step 3: tert-butyl 4-((4-(1-(2,6-dioxopiperidin-3-yl)-3-methyl-2-oxo-2,3-dihydro-1H-benzo[d]imidazol-5-yl)piperidin-1-yl)methyl)-[1,4′-bipiperidine]-1′-carboxylate (91c)

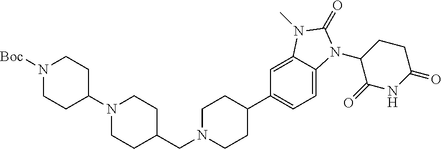 [SMILES: Cn1c(=O)n(C2CCC(=O)NC2=O)c2ccc(C3CCN(CC4CCN(C5CCN(C(=O)OC(C)(C)C)CC5)CC4)CC3)cc21]

The above crude 91b trifluoroacetate (245 mg) was dissolved in 15 mL of THF, and solid sodium bicarbonate (138 mg, 1.64 mmol) was added. The mixture was stirred at room temperature for 20 min, and then N-Boc-4-piperidone (325 mg, 1.63 mmol) and 0.1 mL of acetic acid were added. The mixture was stirred at room temperature for 60 min, and then sodium triacetoxyborohydride (350 mg, 1.65 mmol) was added. The resulting mixture was reacted at room temperature for 16 h. To the reaction system was slowly added 10 mL of saturated sodium bicarbonate solution, and the mixture was extracted with 50 mL of DCM.

The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 91c (180 mg, two-step yield from compound 91a: 79%).

LCMS m/z=623.4 [M+1]⁺

### Step 4: 3-(5-(1-([1,4′-bipiperidin]-4-ylmethyl)piperidin-4-yl)-3-methyl-2-oxo-2,3-dihydro-1H-benzo[d]imidazol-1-yl)piperidine-2,6-dione (91d) trifluoroacetate

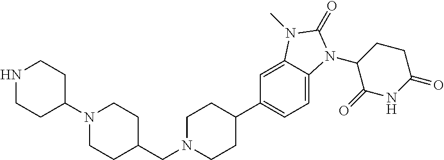 [SMILES: Cn1c(=O)n(C2CCC(=O)NC2=O)c2ccc(C3CCN(CC4CCN(C5CCNCC5)CC4)CC3)cc21]

91c (90 mg, 0.14 mmol) was dissolved in 10 mL of DCM, and 4 mL of trifluoroacetic acid was added. The mixture was stirred at room temperature for 2 h. The reaction liquid was concentrated under reduced pressure, to afford crude 91d trifluoroacetate (110 mg).

LCMS m/z=523.4 [M+1]⁺

### Step 5: 3-chloro-5-(2-(4-((2-(4-((4-(1-(2,6-dioxopiperidin-3-yl)-3-methyl-2-oxo-2,3-dihydro-1H-benzo[d]imidazol-5-yl)piperidin-1-yl)methyl)-[1,4′-bipiperidin]-1′-yl)pyrimidin-4-yl)methoxy)phenyl)propan-2-yl)benzonitrile (Compound 91) trifluoroacetate

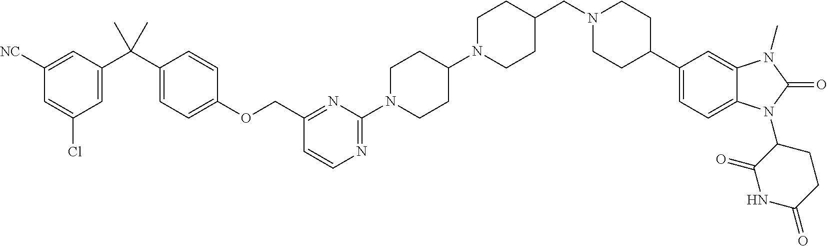 [SMILES: Cn1c(=O)n(C2CCC(=O)NC2=O)c2ccc(C3CCN(CC4CCN(C5CCN(c6nccc(COc7ccc(C(C)(C)c8cc(Cl)cc(C#N)c8)cc7)n6)CC5)CC4)CC3)cc21]

3-chloro-5-(2-(4-((2-chloropyrimidin-4-yl)methoxy)phenyl)propan-2-yl)benzonitrile (9d) (86 mg, 0.21 mmol) was dissolved in 6 mL of DMF, and the above crude 91d trifluoroacetate (110 mg) and solid potassium bicarbonate (116 mg, 1.16 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 91 trifluoroacetate (60 mg).

¹H NMR (400 MHz, DMSO-d₆) δ 11.08 (s, 1H), 8.40 (d, 1H), 7.90-7.80 (m, 1H), 7.70-7.60 (m, 1H), 7.57-7.45 (m, 1H), 7.25-7.13 (m, 2H), 7.13-6.86 (m, 5H), 6.75 (d, 1H), 5.45-5.29 (m, 1H), 4.99 (s, 2H), 4.94-4.75 (m, 2H), 3.73-3.45 (m, 5H), 3.34 (s, 3H), 3.33-3.15 (m, 1H), 3.15-2.80 (m, 9H), 2.78-2.57 (m, 2H), 2.27-1.86 (m, 10H), 1.75-1.35 (m, 10H).

LCMS m/z=884.4 [M+1]⁺

### Example 92

### 3-chloro-5-(2-(4-((2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)pyrimidin-4-yl)methoxy)phenyl)propan-2-yl)-2-methoxybenzonitrile (Compound 92) trifluoroacetate

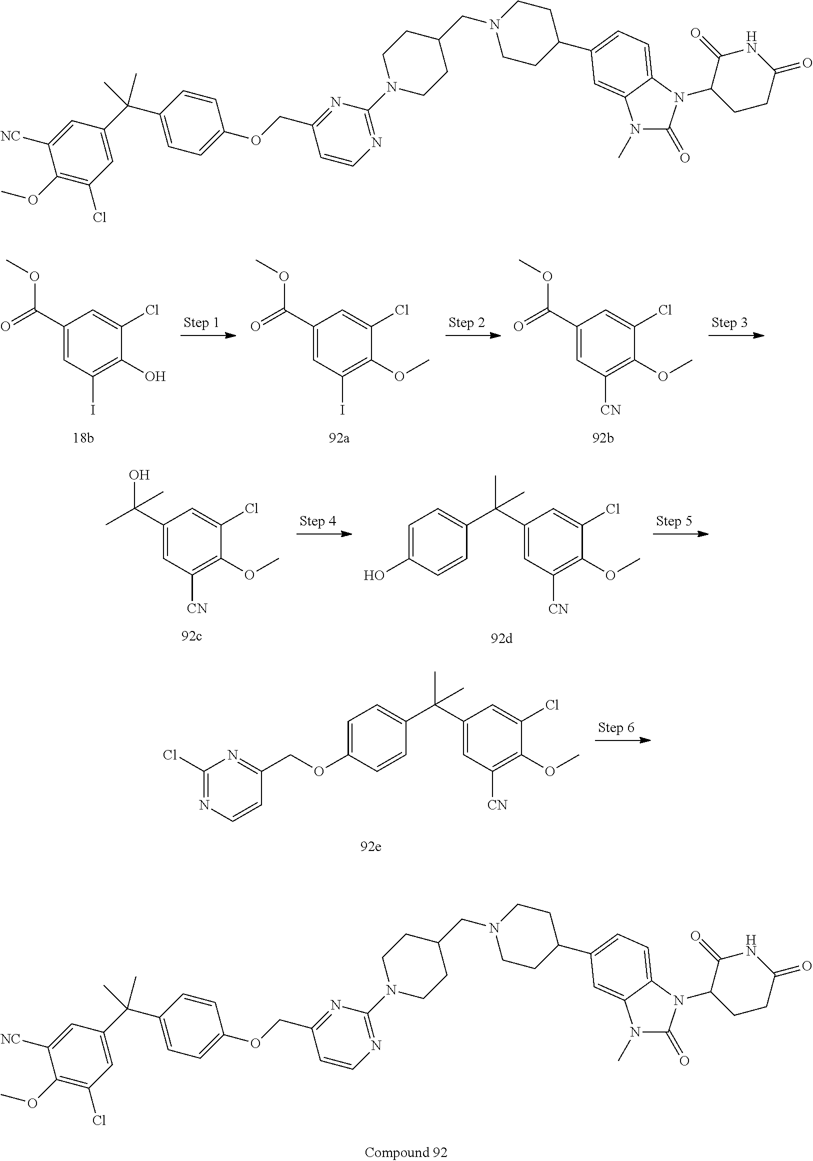

### Step 1: methyl 3-chloro-5-iodo-4-methoxybenzoate (92a)

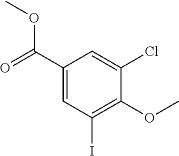

The above crude methyl 3-chloro-4-hydroxy-5-iodobenzoate (18b) (11.05 g) was dissolved in 100 mL of DMF, and iodomethane (9.02 g, 63.55 mmol) and potassium carbonate (10.66 g, 77.1 mmol) were added. The mixture was stirred at room temperature for 16 h. To the reaction liquid was slowly added 200 mL of water, and the mixture was extracted with 200 mL of ethyl acetate. The organic phase was washed with a saturated sodium chloride solution (50 mL×2), dried over anhydrous sodium sulfate, and concentrated under reduced pressure, to afford crude 92a (11.50 g).

### Step 2: methyl 3-chloro-5-cyano-4-methoxybenzoate (92b)

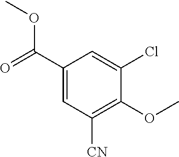

The above crude 92a (11.50 g) was dissolved in 100 mL of N-methylpyrrolidone, and copper cyanide (3.84 g, 42.9 mmol) was added. The mixture was warmed to 160° C. and reacted for 2 h. The reaction liquid was cooled to room temperature and filtered. The filter cake was washed with 100 mL of mixed solvent (ethyl acetate/methanol (v/v)=10:1), and to the filtrate was slowly added 100 mL of water. The mixture was extracted with 200 mL of ethyl acetate, and the organic phase was washed with a saturated sodium chloride solution (50 mL×2), dried over anhydrous sodium sulfate, and concentrated under reduced pressure. The crude product was separated and purified by silica gel column chromatography (ethyl acetate/petroleum ether (v/v)=0:1-1:5), to afford 92b (7.01 g, three-step yield from compound 18a: 79%).

### Step 3: 3-chloro-5-(2-hydroxypropan-2-yl)-2-methoxybenzonitrile (92c)

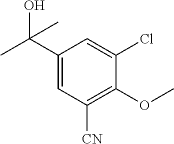

40 mL of 3 mol/L solution of methylmagnesium bromide in 2-methyl tetrahydrofuran was added to 35 mL of tetrahydrofuran, and the mixture was cooled to 0° C. 70 mL of solution of methyl 3-chloro-5-cyano-4-methoxybenzoate (92b) (7.01 g, 31.11 mmol) in tetrahydrofuran was then added dropwise, and the mixture was reacted at 0° C. for further 2 h. At 0° C., to the reaction liquid was added dropwise 200 mL of saturated ammonium chloride solution to quench the reaction. The mixture was extracted with 200 mL of ethyl acetate, washed with 100 mL of saturated sodium chloride solution, dried over anhydrous sodium sulfate, and concentrated under reduced pressure, to afford crude 92c (6.1 g).

### Step 4: 3-chloro-5-(2-(4-hydroxyphenyl)propan-2-yl)-2-methoxybenzonitrile (92d)

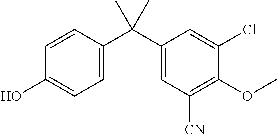 [SMILES: COc1c(Cl)cc(C(C)(C)c2ccc(O)cc2)cc1C#N]

The above crude 92c (6.12 g) was dissolved in 65 mL of carbon tetrachloride, and phenol (2.85 g, 30.3 mmol) was added. The mixture was cooled to 0° C., and boron trifluoride diethyl etherate (7.90 g, 55.65 mmol) was added. The resulting mixture was then warmed to room temperature and reacted for 3 h. To the reaction liquid was slowly added 100 mL of water, and the mixture was extracted with 150 mL of dichloromethane. The organic phase was washed with 100 mL of saturated sodium chloride solution, dried over anhydrous sodium sulfate, and concentrated under reduced pressure. The crude product was separated and purified by silica gel column chromatography (ethyl acetate/petroleum ether (v/v)=0:1-3:7), to afford 92d (4.00 g, two-step yield from compound 92b: 42%).

### Step 5: 3-chloro-5-(2-(4-((2-chloropyrimidin-4-yl)methoxy)phenyl)propan-2-yl)-2-methoxybenzonitrile (92e)

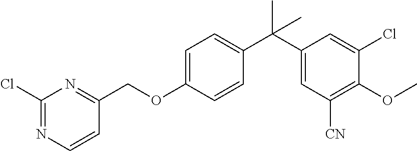 [SMILES: COc1c(Cl)cc(C(C)(C)c2ccc(OCc3ccnc(Cl)n3)cc2)cc1C#N]

92d (2.82 g, 9.34 mmol) was dissolved in 70 mL of acetonitrile, and 2-chloro-4-(chloromethyl)pyrimidine (1.53 g, 9.38 mmol) and potassium carbonate (2.59 g, 18.74 mmol) were successively added. The mixture was warmed to 70° C. and reacted for 20 h. The reaction liquid was cooled to room temperature, filtered, and concentrated under reduced pressure. The crude product was separated and purified by silica gel column chromatography (ethyl acetate/petroleum ether (v/v)=0:1-3:7), to afford 92e (3.66 g, yield: 91%).

### Step 6: 3-chloro-5-(2-(4-((2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)pyrimidin-4-yl)methoxy)phenyl)propan-2-yl)-2-methoxybenzonitrile (Compound 92) trifluoroacetate

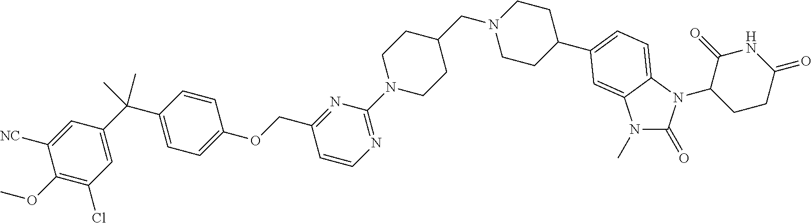 [SMILES: COc1c(Cl)cc(C(C)(C)c2ccc(OCc3ccnc(N4CCC(CN5CCC(c6ccc7c(c6)n(C)c(=O)n7C6CCC(=O)NC6=O)CC5)CC4)n3)cc2)cc1C#N]

92e (155 mg, 0.36 mmol) was dissolved in 6 mL of DMF, and the above crude 91b trifluoroacetate (160 mg) and solid potassium bicarbonate (144 mg, 1.44 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 92 trifluoroacetate (35 mg).

¹H NMR (400 MHz, DMSO-d₆) δ 11.08 (s, 1H), 8.37 (d, 1H), 7.64-7.53 (m, 2H), 7.24-7.14 (m, 2H), 7.11-7.00 (m, 2H), 7.00-6.85 (m, 3H), 6.69 (d, 1H), 5.45-5.27 (m, 1H), 4.98 (s, 2H), 4.77-4.56 (m, 2H), 3.96 (s, 3H), 3.70-3.57 (m, 2H), 3.39-3.22 (m, 4H), 3.15-2.82 (m, 7H), 2.80-2.58 (m, 2H), 2.28-1.76 (m, 8H), 1.63 (s, 6H), 1.28-1.11 (m, 2H).

LCMS m/z=831.3 [M+1]⁺

### Example 93

### 3-chloro-5-(2-(4-((2-(9-((4-(1-(2,6-dioxopiperidin-3-yl)-3-methyl-2-oxo-2,3-dihydro-1H-benzo[d]imidazol-5-yl)piperidin-1-yl)methyl)-3-azaspiro[5.5]undecan-3-yl)pyrimidin-4-yl)methoxy)phenyl)propan-2-yl)-2-methoxybenzonitrile (Compound 93) trifluoroacetate

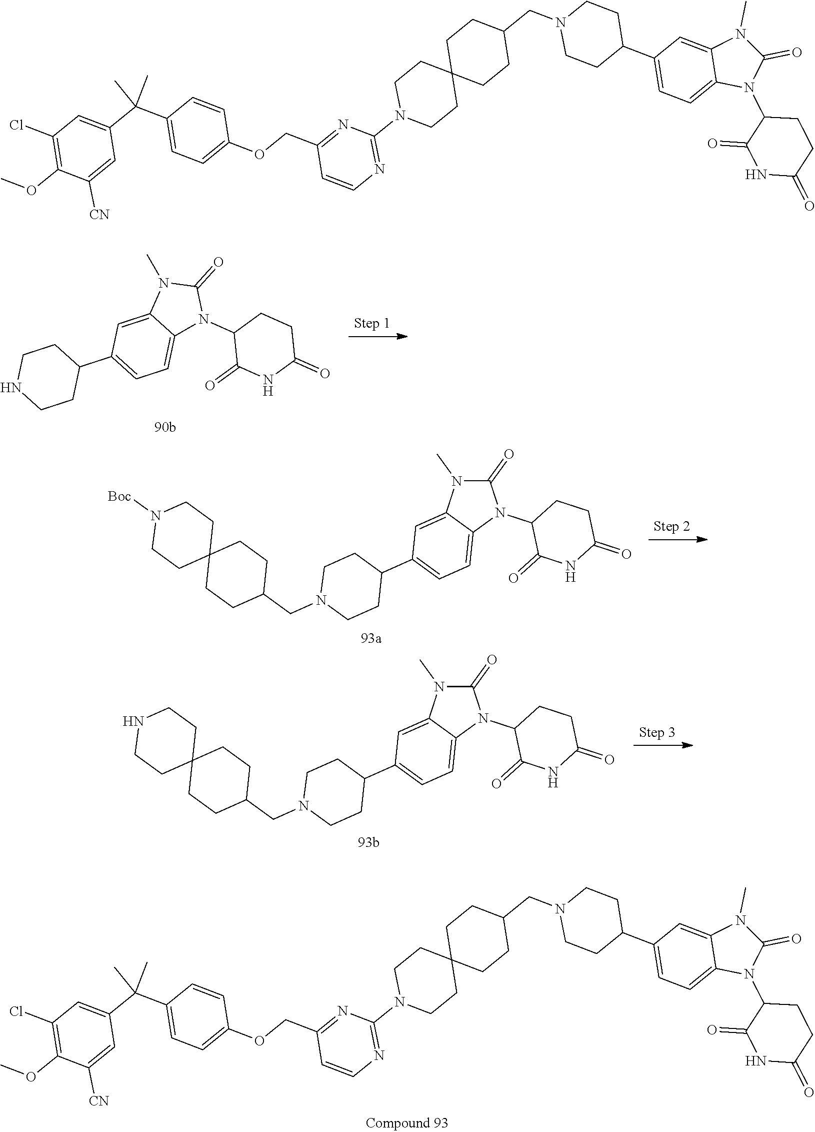 [SMILES: COc1c(Cl)cc(C(C)(C)c2ccc(OCc3ccnc(N4CCC5(CCC(CN6CCC(c7ccc8c(c7)n(C)c(=O)n8C7CCC(=O)NC7=O)CC6)CC5)CC4)n3)cc2)cc1C#N ; Cn1c(=O)n(C2CCC(=O)NC2=O)c2ccc(C3CCNCC3)cc21 ; Cn1c(=O)n(C2CCC(=O)NC2=O)c2ccc(C3CCN(CC4CCC5(CC4)CCN(C(=O)OC(C)(C)C)CC5)CC3)cc21 ; Cn1c(=O)n(C2CCC(=O)NC2=O)c2ccc(C3CCN(CC4CCC5(CCNCC5)CC4)CC3)cc21 ; COc1c(Cl)cc(C(C)(C)c2ccc(OCc3ccnc(N4CCC5(CCC(CN6CCC(c7ccc8c(c7)n(C)c(=O)n8C7CCC(=O)NC7=O)CC6)CC5)CC4)n3)cc2)cc1C#N]

### Step 1: tert-butyl 9-((4-(1-(2,6-dioxopiperidin-3-yl)-3-methyl-2-oxo-2,3-dihydro-1H-benzo[d]imidazol-5-yl)piperidin-1-yl)methyl)-3-azaspiro[5.5]undecane-3-carboxylate (93a)

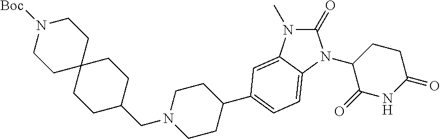 [SMILES: Cn1c(=O)n(C2CCC(=O)NC2=O)c2ccc(C3CCN(CC4CCC5(CC4)CCN(C(=O)OC(C)(C)C)CC5)CC3)cc21]

The above crude 90b trifluoroacetate (180 mg) was dissolved in 15 mL of THF, and solid sodium bicarbonate (57 mg, 0.68 mmol) was added. The mixture was stirred at room temperature for 20 min, and then tert-butyl 9-formyl-3-azaspiro[5.5]undecane-3-carboxylate (see WO 2014059232 for the synthetic method) (204 mg, 0.72 mmol) and 0.1 mL of acetic acid were added. The mixture was stirred at room temperature for 60 min, and then sodium triacetoxyborohydride (144 mg, 0.68 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 93a (120 mg, two-step yield from compound 90a: 58%).

LCMS m/z=608.4 [M+1]⁺

### Step 2: 3-(5-(1-((3-azaspiro[5.5]undecan-9-yl)methyl)piperidin-4-yl)-3-methyl-2-oxo-2,3-dihydro-1H-benzo[d]imidazol-1-yl)piperidine-2,6-dione (93b) trifluoroacetate

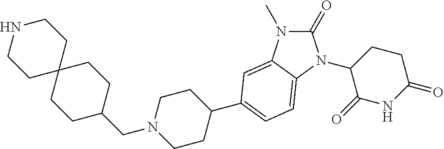 [SMILES: Cn1c(=O)n(C2CCC(=O)NC2=O)c2ccc(C3CCN(CC4CCC5(CCNCC5)CC4)CC3)cc21]

93a (120 mg, 0.20 mmol) was dissolved in 10 mL of DCM, and 4 mL of trifluoroacetic acid was added. The mixture was stirred at room temperature for 2 h. The reaction liquid was concentrated under reduced pressure, to afford crude 93b trifluoroacetate (160 mg).

LCMS m/z=508.3 [M+1]⁺

### Step 3: 3-chloro-5-(2-(4-((2-(9-((4-(1-(2,6-dioxopiperidin-3-yl)-3-methyl-2-oxo-2,3-dihydro-1H-benzo[d]imidazol-5-yl)piperidin-1-yl)methyl)-3-azaspiro[5.5]undecan-3-yl)pyrimidin-4-yl)methoxy)phenyl)propan-2-yl)-2-methoxybenzonitrile (Compound 93) trifluoroacetate

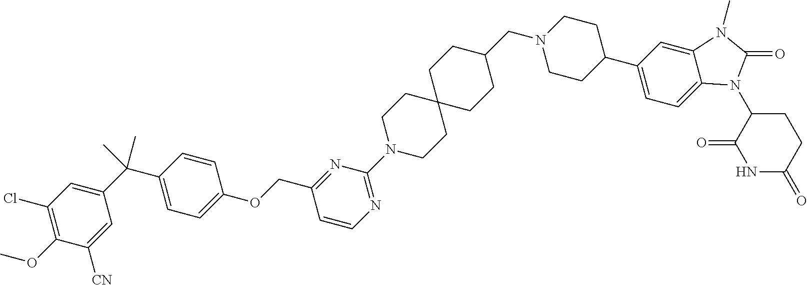 [SMILES: COc1c(Cl)cc(C(C)(C)c2ccc(OCc3ccnc(N4CCC5(CCC(CN6CCC(c7ccc8c(c7)n(C)c(=O)n8C7CCC(=O)NC7=O)CC6)CC5)CC4)n3)cc2)cc1C#N]

92e (135 mg, 0.32 mmol) was dissolved in 6 mL of DMF, and the above crude 3-(5-(1-((3-azaspiro[5.5]undecan-9-yl)methyl)piperidin-4-yl)-3-methyl-2-oxo-2,3-dihydro-1H-benzo[d]imidazol-1-yl)piperidine-2,6-dione (93b) trifluoroacetate (160 mg) and solid potassium bicarbonate (208 mg, 2.08 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 93 trifluoroacetate (10 mg).

¹H NMR (400 MHz, DMSO-d₆) δ 11.08 (s, 1H), 8.34 (d, 1H), 7.64-7.53 (m, 2H), 7.24-6.85 (m, 7H), 6.66 (d, 1H), 5.43-5.30 (m, 1H), 4.97 (s, 2H), 3.96 (s, 3H), 3.80-3.68 (m, 4H), 3.66-3.60 (m, 2H), 3.35 (s, 3H), 3.17-2.80 (m, 6H), 2.80-2.56 (m, 2H), 2.15-1.93 (m, 5H), 1.93-1.43 (m, 13H), 1.40-1.12 (m, 6H).

LCMS m/z=899.4 [M+1]⁺

### Example 94

### 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-ethoxybenzonitrile (Compound 94) trifluoroacetate

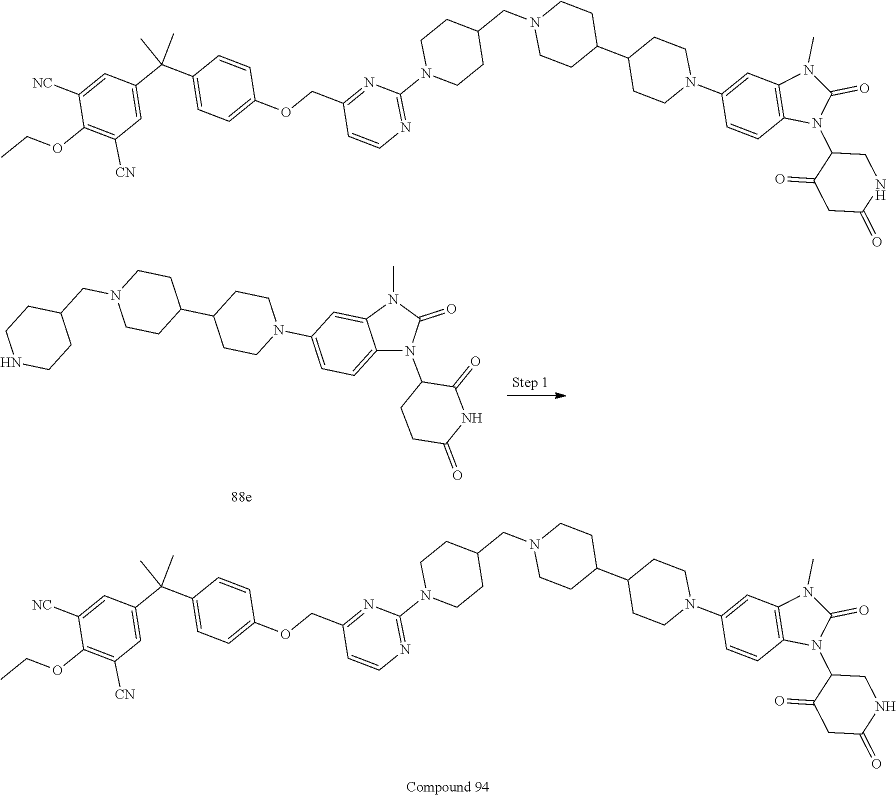 [SMILES: CCOc1c(C#N)cc(C(C)(C)c2ccc(OCc3ccnc(N4CCC(CN5CCC(C6CCN(c7ccc8c(c7)n(C)c(=O)n8C7CNC(=O)CC7=O)CC6)CC5)CC4)n3)cc2)cc1C#N ; Cn1c(=O)n(C2CCC(=O)NC2=O)c2ccc(N3CCC(C4CCN(CC5CCNCC5)CC4)CC3)cc21 ; CCOc1c(C#N)cc(C(C)(C)c2ccc(OCc3ccnc(N4CCC(CN5CCC(C6CCN(c7ccc8c(c7)n(C)c(=O)n8C7CNC(=O)CC7=O)CC6)CC5)CC4)n3)cc2)cc1C#N]

The above crude 88e trifluoroacetate (0.5 g) was dissolved in 20 mL of DMF, and solid potassium bicarbonate (0.47 g, 4.7 mmol) was added. The mixture was stirred at room temperature for 0.5 h, and then 3-chloro-5-(2-(4-((2-chloropyrimidin-4-yl)methoxy)phenyl)propan-2-yl)-2-ethoxybenzonitrile (18g) (0.84 g, 1.90 mmol) was added. The resulting mixture was warmed to 80° C. and stirred for 4 h. The reaction liquid was cooled to room temperature, and 20 mL of water was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 94 trifluoroacetate (0.1 g).

¹H NMR (400 MHz, DMSO-d₆) δ 11.09 (s, 1H), 8.36 (d, 1H), 7.63-7.52 (m, 2H), 7.45-7.00 (m, 5H), 6.98-6.90 (m, 2H), 6.69 (d, 1H), 5.42-5.32 (m, 1H), 4.97 (s, 2H), 4.72-4.62 (m, 2H), 4.19 (q, 2H), 3.73-3.49 (m, 4H), 3.36 (s, 3H), 3.32-3.08 (m, 2H), 3.04-2.81 (m, 7H), 2.79-2.56 (m, 2H), 2.25-1.74 (m, 8H), 1.63 (s, 6H), 1.60-1.40 (m, 6H), 1.37 (t, 3H), 1.23-1.08 (m, 2H).

LCMS m/z=310.3 [M/3+1]⁺

### Example 95

### 3-chloro-5-(2-(4-((2-(1′-(1-(2,6-dioxopiperidin-3-yl)-3-methyl-2-oxo-2,3-dihydro-1H-benzo[d]imidazol-5-yl)-[4,4′-bipiperidin]-1-yl)pyrimidin-4-yl)methoxy)phenyl)propan-2-yl)-2-methoxybenzonitrile (Compound 95)

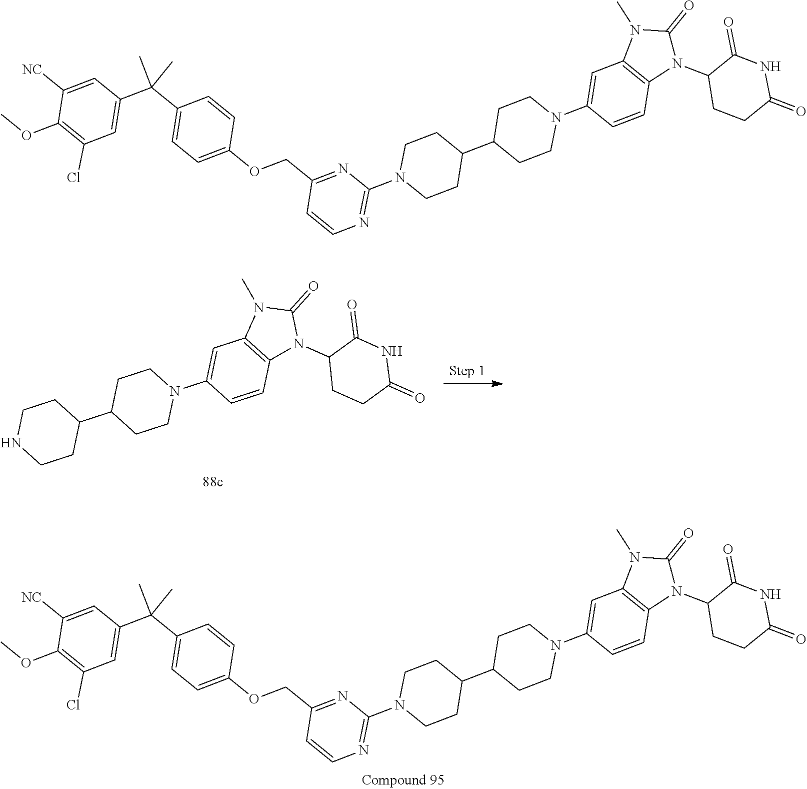 [SMILES: COc1c(Cl)cc(C(C)(C)c2ccc(OCc3ccnc(N4CCC(C5CCN(c6ccc7c(c6)n(C)c(=O)n7C6CCC(=O)NC6=O)CC5)CC4)n3)cc2)cc1C#N ; Cn1c(=O)n(C2CCC(=O)NC2=O)c2ccc(N3CCC(C4CCNCC4)CC3)cc21 ; COc1c(Cl)cc(C(C)(C)c2ccc(OCc3ccnc(N4CCC(C5CCN(c6ccc7c(c6)n(C)c(=O)n7C6CCC(=O)NC6=O)CC5)CC4)n3)cc2)cc1C#N]

The above crude 88c trifluoroacetate (60 mg) was dissolved in 5.0 mL of DMF, and solid potassium bicarbonate (138 mg, 1.38 mmol) was added. The mixture was stirred at room temperature for 0.5 h, and then 3-chloro-5-(2-(4-((2-chloropyrimidin-4-yl)methoxy)phenyl)propan-2-yl)-2-methoxybenzonitrile (92e) (60.0 mg, 0.14 mmol) was added. The resulting mixture was warmed to 80° C. and stirred for 4 h. The reaction liquid was cooled to room temperature, and 50 mL of water was added. The mixture was extracted with ethyl acetate (40 mL×3), and the organic phase was washed with 50 mL of saturated sodium chloride solution, dried over anhydrous sodium sulfate, and concentrated under reduced pressure. The crude product was then separated and purified by a thick preparative plate (developing agent: ethyl acetate/petroleum ether (v/v)=4:1), to afford compound 95 (37 mg, two-step yield from compound 88b: 48%).

¹H NMR (400 MHz, CDCl₃) δ 8.34-8.26 (m, 1H), 8.07-7.97 (m, 1H), 7.45-7.39 (m, 1H), 7.34-7.29 (m, 1H), 7.28-7.22 (m, 1H), 7.14-7.06 (m, 2H), 6.92-6.85 (m, 2H), 6.82-6.60 (m, 3H), 5.26-5.13 (m, 1H), 4.95 (s, 2H), 4.90-4.78 (m, 2H), 4.04 (s, 3H), 3.75-3.54 (m, 2H), 3.43 (s, 3H), 3.05-2.60 (m, 5H), 2.30-2.18 (m, 1H), 2.02-1.75 (m, 4H), 1.63 (s, 6H), 1.60-1.45 (m, 4H), 1.33-1.18 (m, 4H).

LCMS m/z=817.3 [M+1]⁺

### Example 96

### 3-chloro-5-(2-(4-((2-(9-(1-(2,6-dioxopiperidin-3-yl)-3-methyl-2-oxo-2,3-dihydro-1H-benzo[d]imidazol-5-yl)-3,9-diazaspiro[5.5]undecan-3-yl)pyrimidin-4-yl)methoxy)phenyl) propan-2-yl)-2-methoxybenzonitrile (Compound 96)

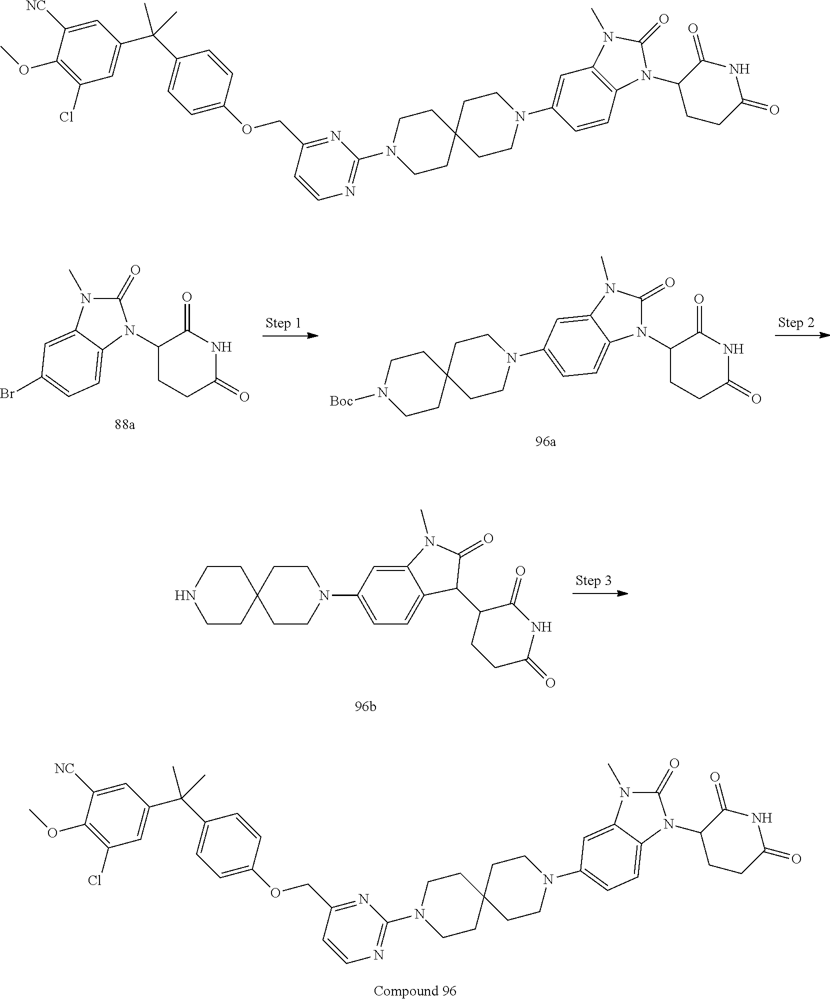 [SMILES: COc1c(Cl)cc(C(C)(C)c2ccc(OCc3ccnc(N4CCC5(CCN(c6ccc7c(c6)n(C)c(=O)n7C6CCC(=O)NC6=O)CC5)CC4)n3)cc2)cc1C#N ; Cn1c(=O)n(C2CCC(=O)NC2=O)c2ccc(Br)cc21 ; Cn1c(=O)n(C2CCC(=O)NC2=O)c2ccc(N3CCC4(CCN(C(=O)OC(C)(C)C)CC4)CC3)cc21 ; CN1C(=O)C(C2CCC(=O)NC2=O)c2ccc(N3CCC4(CCNCC4)CC3)cc21 ; COc1c(Cl)cc(C(C)(C)c2ccc(OCc3ccnc(N4CCC5(CCN(c6ccc7c(c6)n(C)c(=O)n7C6CCC(=O)NC6=O)CC5)CC4)n3)cc2)cc1C#N]

### Step 1: tert-butyl 9-(1-(2,6-dioxopiperidin-3-yl)-3-methyl-2-oxo-2,3-dihydro-1H-benzo[d]imidazol-5-yl)-3,9-diazaspiro[5.5]undecane-3-carboxylate (96a)

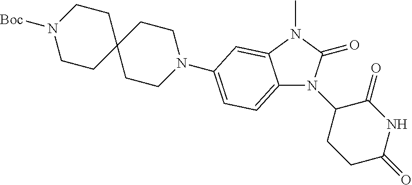 [SMILES: Cn1c(=O)n(C2CCC(=O)NC2=O)c2ccc(N3CCC4(CCN(C(=O)OC(C)(C)C)CC4)CC3)cc21]

To a 50 mL three-necked flask were successively added 3-(5-bromo-3-methyl-2-oxo-2,3-dihydro-1H-benzo[d]imidazol-1-yl)piperidine-2,6-dione (88a) (see WO 2020113233 for the synthetic method) (260.0 mg, 0.77 mmol), tert-butyl 3,9-diazaspiro[5.5]undecane-3-carboxylate (230.0 mg, 0.904 mmol), RuPhos-Pd-G3 (CAS: 1445085-77-7) (120.0 mg, 0.139 mmol) and RuPhos (68.6 mg, 0.147 mmol), and the mixture was subjected to nitrogen replacement 3 times. Under nitrogen atmosphere, to the reaction system were respectively added toluene (5.0 mL) and 1 mol/L solution of LiHMDS in tetrahydrofuran (4.0 mL, 4.0 mmol), and the mixture was warmed to 80° C. and reacted for 2 h. The reaction liquid was cooled to room temperature, and a saturated ammonium chloride solution (5.0 mL) was slowly added. The mixture was extracted with ethyl acetate (15×3 mL), and the organic phase was washed with 30 mL of saturated sodium chloride solution, dried over anhydrous sodium sulfate, and concentrated under reduced pressure.

The crude product was separated and purified by silica gel column chromatography (ethyl acetate/petroleum ether (v/v)=0:1-1:0), to afford 96a (90 mg, yield: 23%).

### Step 2: 3-(3-methyl-2-oxo-5-(3,9-diazaspiro[5.5]undecan-3-yl)-2,3-dihydro-1H-benzo[d]imidazol-1-yl)piperidine-2,6-dione (96b) trifluoroacetate

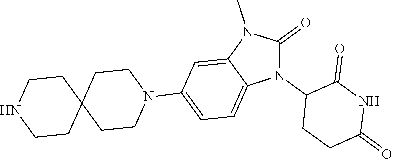 [SMILES: Cn1c(=O)n(C2CCC(=O)NC2=O)c2ccc(N3CCC4(CCNCC4)CC3)cc21]

To a reaction flask were respectively added 96a (90.0 mg, 0.176 mmol), trifluoroacetic acid (2.0 mL) and dichloromethane (4.0 mL), and the mixture was reacted at room temperature for 1 h. The reaction liquid was concentrated under reduced pressure, to afford crude 96b trifluoroacetate (70 mg).

### Step 3: 3-chloro-5-(2-(4-((2-(9-(1-(2,6-dioxopiperidin-3-yl)-3-methyl-2-oxo-2,3-dihydro-1H-benzo[d]imidazol-5-yl)-3,9-diazaspiro[5.5]undecan-3-yl)pyrimidin-4-yl)methoxy)phenyl)propan-2-yl)-2-methoxybenzonitrile (Compound 96)

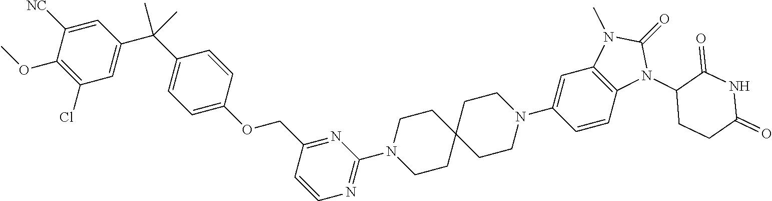 [SMILES: COc1c(Cl)cc(C(C)(C)c2ccc(OCc3ccnc(N4CCC5(CCN(c6ccc7c(c6)n(C)c(=O)n7C6CCC(=O)NC6=O)CC5)CC4)n3)cc2)cc1C#N]

The above crude 96b trifluoroacetate (70 mg) was dissolved in 5.0 mL of DMF, and solid potassium bicarbonate (150 mg, 1.50 mmol) was added. The mixture was stirred at room temperature for 0.5 h, and then 92e (70.0 mg, 0.16 mmol) was added. The resulting mixture was warmed to 80° C. and stirred for 4 h. The reaction liquid was cooled to room temperature, and 50 mL of water was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then separated and purified by a thick preparative plate (developing agent: ethyl acetate/petroleum ether (v/v)=5.7:1), to afford compound 96 (35 mg, two-step yield from compound 96a: 25%).

¹H NMR (400 MHz, CDCl₃) δ 8.31 (d, 1H), 8.07 (s, 1H), 7.43 (d, 1H), 7.31 (d, 1H), 7.28-7.24 (m, 1H), 7.13-7.06 (m, 2H), 6.92-6.85 (m, 2H), 6.82-6.62 (m, 3H), 5.19 (dd, 1H), 4.95 (s, 2H), 4.04 (s, 3H), 3.90-3.78 (m, 4H), 3.43 (s, 3H), 3.37-3.05 (m, 4H), 3.04-2.60 (m, 3H), 2.29-2.18 (m, 1H), 2.05-1.70 (m, 4H), 1.70-1.45 (m, 10H).

LCMS m/z=803.3 [M+1]⁺

### Example 97

### 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-methoxybenzonitrile (Compound 97)

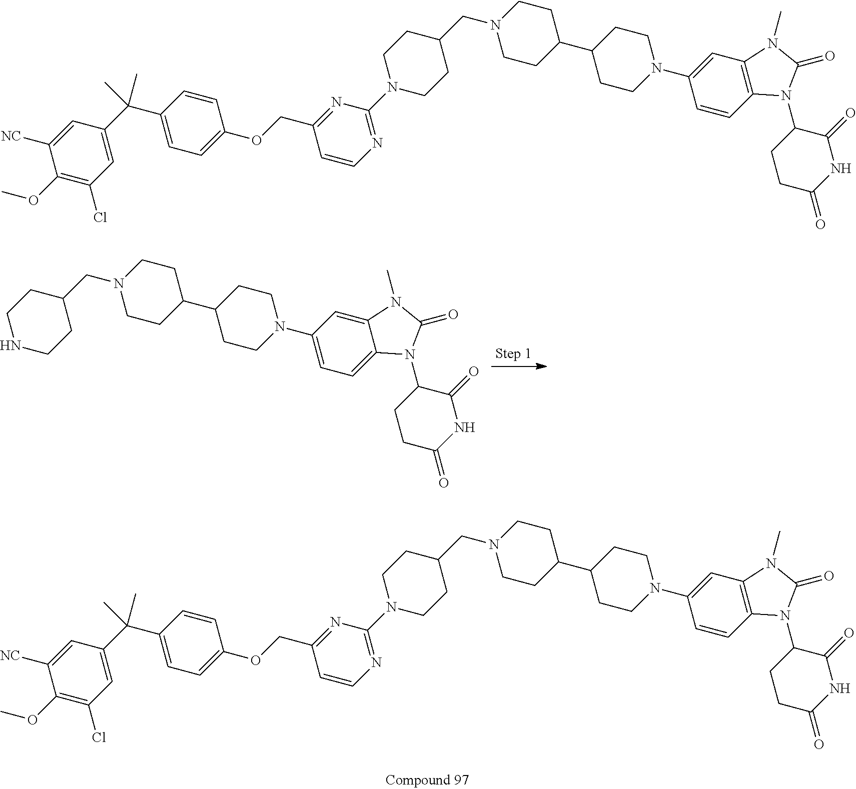 [SMILES: COc1c(Cl)cc(C(C)(C)c2ccc(OCc3ccnc(N4CCC(CN5CCC(C6CCN(c7ccc8c(c7)n(C)c(=O)n8C7CCC(=O)NC7=O)CC6)CC5)CC4)n3)cc2)cc1C#N ; Cn1c(=O)n(C2CCC(=O)NC2=O)c2ccc(N3CCC(C4CCN(CC5CCNCC5)CC4)CC3)cc21 ; COc1c(Cl)cc(C(C)(C)c2ccc(OCc3ccnc(N4CCC(CN5CCC(C6CCN(c7ccc8c(c7)n(C)c(=O)n8C7CCC(=O)NC7=O)CC6)CC5)CC4)n3)cc2)cc1C#N]

The above crude 88e trifluoroacetate (2.60 g) was dissolved in 20 mL of DMF, and solid potassium bicarbonate (3.0 g, 30 mmol) was added. The mixture was stirred at room temperature for 0.5 h, and then 92e (2.00 g, 4.67 mmol) was added. The resulting mixture was warmed to 80° C. and stirred for 4 h. The reaction liquid was cooled to room temperature, and 20 mL of water was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 97 trifluoroacetate (1.25 g).

LCMS m/z=457.8 [M/2+1]+

3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-methoxybenzonitrile (Compound 97) trifluoroacetate (200 mg) was dissolved in 60 mL of dichloromethane, and 30 mL of 25% ammonia water was added. The mixture was stirred vigorously, and the organic phase was separated, washed with 25% ammonia water (30 mL×3), dried over anhydrous sodium sulfate, and concentrated under reduced pressure, to afford 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-methoxybenzonitrile (Compound 97) (160 mg, two-step yield from compound 92e: 23%).

¹H NMR (400 MHz, CDCl₃) δ 8.38 (br.s, 1H), 8.29 (d, 1H), 7.42 (d, 1H), 7.32 (d, 1H), 7.13-7.05 (m, 2H), 6.92-6.83 (m, 2H), 6.74-6.58 (m, 4H), 5.18 (dd, 1H), 4.94 (s, 2H), 4.83-4.70 (m, 2H), 4.04 (s, 3H), 3.64-3.52 (m, 2H), 3.40 (s, 3H), 3.05-2.55 (m, 9H), 2.34-2.06 (m, 3H), 2.00-1.67 (m, 9H), 1.63 (s, 6H), 1.52-1.04 (m, 8H).

LCMS m/z=457.8 [M/2+1]⁺

### Example 98

### 3-chloro-2-(2-chloroethoxy)-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)benzonitrile (Compound 98) trifluoroacetate

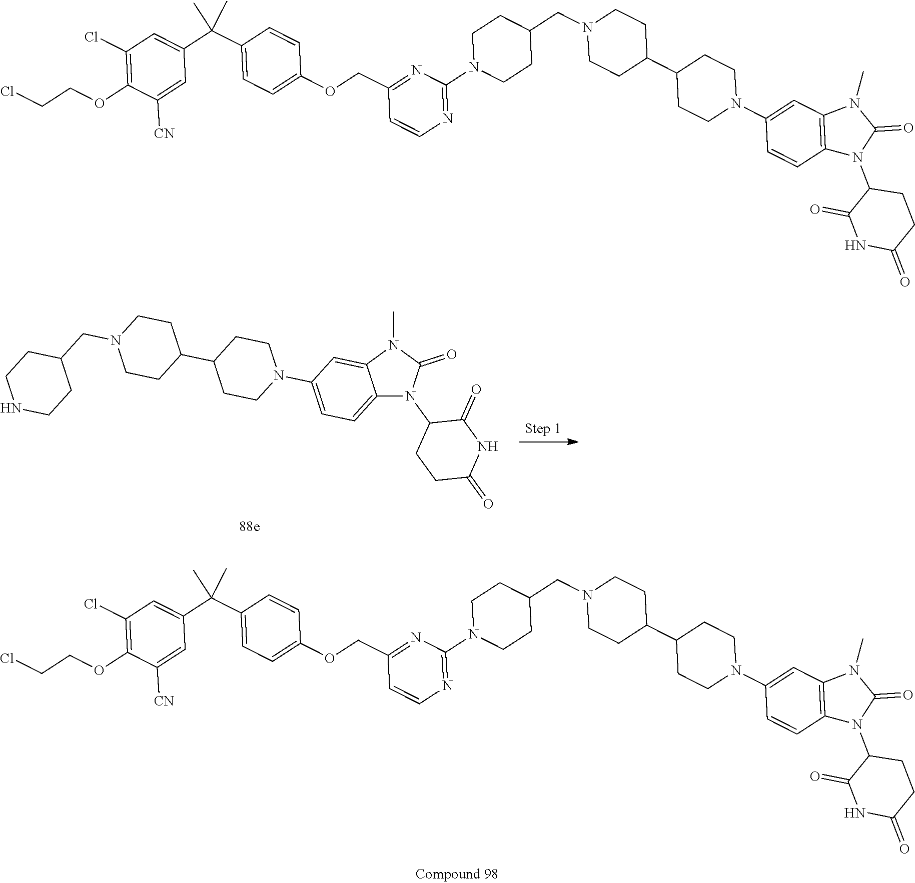 [SMILES: Cn1c(=O)n(C2CCC(=O)NC2=O)c2ccc(N3CCC(C4CCN(CC5CCN(c6nccc(COc7ccc(C(C)(C)c8cc(Cl)c(OCCCl)c(C#N)c8)cc7)n6)CC5)CC4)CC3)cc21 ; Cn1c(=O)n(C2CCC(=O)NC2=O)c2ccc(N3CCC(C4CCN(CC5CCNCC5)CC4)CC3)cc21 ; Cn1c(=O)n(C2CCC(=O)NC2=O)c2ccc(N3CCC(C4CCN(CC5CCN(c6nccc(COc7ccc(C(C)(C)c8cc(Cl)c(OCCCl)c(C#N)c8)cc7)n6)CC5)CC4)CC3)cc21]

The above crude 88e trifluoroacetate (120 mg) was dissolved in 2 mL of DMF, and solid potassium bicarbonate (84 mg, 0.84 mmol) was added. The mixture was stirred at room temperature for 0.5 h, and then 3-chloro-2-(2-chloroethoxy)-5-(2-(4-((2-chloropyrimidin-4-yl)methoxy)phenyl)propan-2-yl)benzonitrile (4a) (see WO 2020081999 for the synthetic method) (45 mg, 0.094 mmol) was added. The resulting mixture was warmed to 80° C. and stirred for 4 h. The reaction liquid was cooled to room temperature, and 5 mL of water was added. The mixture was extracted with ethyl acetate (10 mL×3), and the organic phase was washed with 10 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 98 trifluoroacetate (40 mg).

¹H NMR (400 MHz, DMSO-d₆) δ 11.09 (s, 1H), 8.36 (d, 1H), 7.64-7.54 (m, 2H), 7.45-6.85 (m, 7H), 6.69 (d, 1H), 5.42-5.30 (m, 1H), 4.98 (s, 2H), 4.75-4.60 (m, 2H), 4.45-4.38 (m, 2H), 3.99-3.91 (m, 2H), 3.72-3.51 (m, 4H), 3.35 (s, 3H), 3.30-3.06 (m, 2H), 3.03-2.83 (m, 7H), 2.78-2.57 (m, 2H), 2.25-1.75 (m, 8H), 1.70-1.35 (m, 12H), 1.26-1.05 (m, 2H).

LCMS m/z=481.8 [M/2+1]⁺

### Example 99

### 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl) methoxy)phenyl)propan-2-yl)-2-(methoxy-d3)benzonitrile (Compound 99) trifluoroacetate

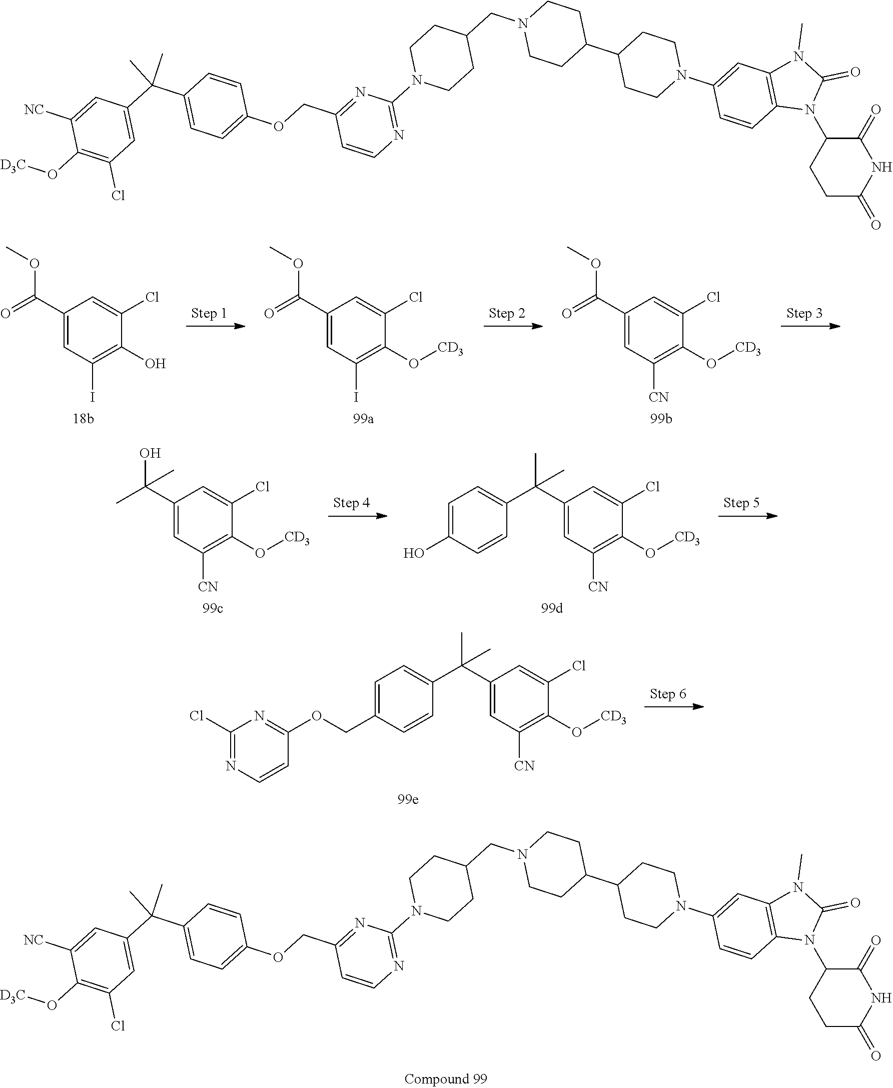

### Step 1: methyl 3-chloro-5-iodo-4-(methoxy-d₃)benzoate (99a)

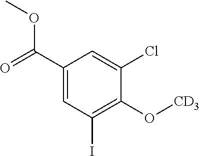 [SMILES: [2H]C([2H])([2H])Oc1c(Cl)cc(C(=O)OC)cc1I]

The above crude methyl 3-chloro-4-hydroxy-5-iodobenzoate (18b) (5.0 g) was dissolved in 20 mL of DMF, and deuterated iodomethane (3.48 g, 24 mmol) and potassium carbonate (3.32 g, 24 mmol) were added. The mixture was stirred at room temperature for 16 h. To the reaction liquid was slowly added 50 mL of water, and the mixture was extracted with 50 mL of ethyl acetate. The organic phase was washed with a saturated sodium chloride solution (50 mL×2), dried over anhydrous sodium sulfate, and concentrated under reduced pressure. The crude product was separated and purified by silica gel column chromatography (petroleum ether/ethyl acetate (v/v)=100:1-10:1), to afford 99a (5.0 g, two-step yield from compound 18a: 85%).

### Step 2: methyl 3-chloro-5-cyano-4-(methoxy-d₃)benzoate (99b)

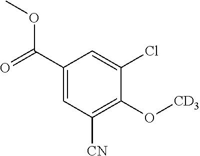 [SMILES: [2H]C([2H])([2H])Oc1c(Cl)cc(C(=O)OC)cc1C#N]

Methyl 3-chloro-5-iodo-4-(methoxy-d₃)benzoate (99a) (5.0 g, 15.2 mmol) was dissolved in 20 mL of N-methylpyrrolidone, and copper cyanide (1.33 g, 14.85 mmol) was added. The mixture was warmed to 160° C. and reacted for 2 h. The reaction liquid was cooled to room temperature and filtered. The filter cake was washed with 100 mL of mixed solvent (ethyl acetate/methanol (v/v)=10:1), and to the filtrate was slowly added 30 mL of water. The mixture was extracted with 50 mL of ethyl acetate, and the organic phase was washed with a saturated sodium chloride solution (20 mL×3), dried over anhydrous sodium sulfate, and concentrated under reduced pressure. The crude product was separated and purified by silica gel column chromatography (petroleum ether/ethyl acetate (v/v)=100:0-5:1), to afford 99b (2.8 g, yield: 81%1).

### Step 3: 3-chloro-5-(2-hydroxypropan-2-yl)-2-(methoxy-d₃)benzonitrile (99c)

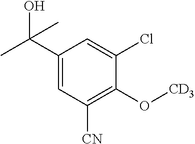 [SMILES: [2H]C([2H])([2H])Oc1c(Cl)cc(C(C)(C)O)cc1C#N]

12 mL of 3 mol/L solution of methylmagnesium bromide in 2-methyl tetrahydrofuran was added to 10 mL of tetrahydrofuran, and the mixture was cooled to 0° C. A solution of 99b (2.0 g, 8.75 mmol) in tetrahydrofuran (20 mL) was then added dropwise, and the mixture was reacted at 0° C. for further 2 h. To the reaction liquid was added dropwise 60 mL of saturated ammonium chloride solution, and the mixture was extracted with 60 mL of ethyl acetate, washed with 60 mL of saturated sodium chloride solution, dried over anhydrous sodium sulfate, and concentrated under reduced pressure. The crude product was separated and purified by silica gel column chromatography (petroleum ether/ethyl acetate (v/v)=100:0-10:1), to afford 99c (710 mg, yield: 35%).

Step 4: 3-chloro-5-(2-(4-hydroxyphenyl)propan-2-yl)-2-(methoxy-d)benzonitrile (99d)

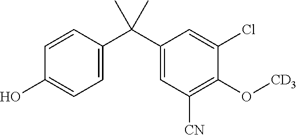 [SMILES: [2H]C([2H])([2H])Oc1c(Cl)cc(C(C)(C)c2ccc(O)cc2)cc1C#N]

99c (710 mg, 3.1 mmol) was dissolved in 10 mL of carbon tetrachloride, and phenol (350 mg, 3.72 mmol) was added. The mixture was cooled to 0° C., and boron trifluoride diethyl etherate (880 mg, 6.2 mmol) was added. The resulting mixture was then warmed to room temperature and reacted for 3 h. To the reaction liquid was slowly added 10 mL of water, and the mixture was extracted with 20 mL of dichloromethane. The organic phase was washed with 20 mL of saturated sodium chloride solution, dried over anhydrous sodium sulfate, and concentrated under reduced pressure. The crude product was separated and purified by silica gel column chromatography (petroleum ether/ethyl acetate (v/v)=100: 0-3:1), to afford 99d (800 mg, yield: 85%).

Step 5: 3-chloro-5-(2-(4-((2-chloropyrimidin-4-yl)methoxy)phenyl)propan-2-yl)-2-(methoxy-d₃)benzonitrile (99e)

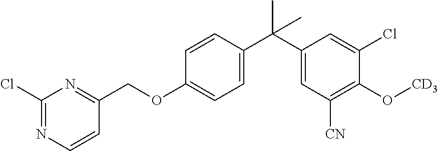 [SMILES: [2H]C([2H])([2H])Oc1c(Cl)cc(C(C)(C)c2ccc(OCc3ccnc(Cl)n3)cc2)cc1C#N]

99d (800 mg, 2.62 mmol) was dissolved in 30 mL of acetonitrile, and 2-chloro-4-(chloromethyl)pyrimidine (470 mg, 2.88 mmol) and potassium carbonate (1.09 g, 7.89 mmol) were successively added. The mixture was warmed to 70° C. and reacted for 20 h. The reaction liquid was cooled to room temperature, filtered, and concentrated under reduced pressure. The crude product was separated and purified by silica gel column chromatography (ethyl acetate/petroleum ether (v/v)=0:1-3:7), to afford 99e (750 mg, yield: 67%).

LCMS m/z=431.1 [M+1]⁺

### Step 6: 3-chloro-5-(2-(4-((2-(4-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-(methoxy-d3)benzonitrile (Compound 99) trifluoroacetate

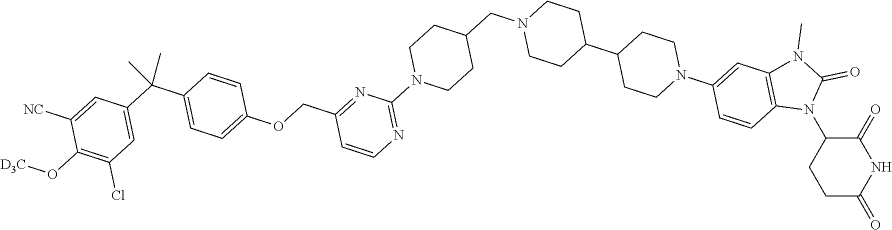 [SMILES: [2H]C([2H])([2H])Oc1c(Cl)cc(C(C)(C)c2ccc(OCc3ccnc(N4CCC(CN5CCC(C6CCN(c7ccc8c(c7)n(C)c(=O)n8C7CCC(=O)NC7=O)CC6)CC5)CC4)n3)cc2)cc1C#N]

The above crude 88e trifluoroacetate (110 mg) was dissolved in 5 mL of DMF, and solid potassium bicarbonate (126 mg, 1.26 mmol) was added. The mixture was stirred at room temperature for 0.5 h, and then 99e (100 mg, 0.23 mmol) was added. The resulting mixture was warmed to 80° C. and stirred for 4 h. The reaction liquid was cooled to room temperature, and 10 mL of water was added. The mixture was extracted with ethyl acetate (20 mL×3), and the organic phase was washed with 10 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 99 trifluoroacetate (75 mg).

¹H NMR (400 MHz, DMSO-d₆) δ 11.10 (s, 1H), 8.36 (d, 1H), 7.68-7.50 (m, 1H), 7.50-7.30 (m, 2H), 7.30-7.05 (m, 4H), 7.02-6.85 (m, 2H), 6.69 (d, 1H), 5.45-5.33 (m, 1H), 5.01-4.93 (m, 2H), 4.73-4.61 (m, 2H), 3.72-3.51 (m, 4H), 3.45-3.18 (m, 5H), 3.14-2.82 (m, 7H), 2.79-2.59 (m, 2H), 2.22-1.75 (m, 8H), 1.73-1.38 (m, 12H), 1.23-1.08 (m, 2H).

LCMS m/z=459.3 [M/2+1]⁺

### Example 100

### 3-chloro-5-(2-(4-((2-(9-(1-((1-(2,6-dioxopiperidin-3-yl)-3-methyl-2-oxo-2,3-dihydro-1H-benzo[d]imidazol-4-yl)methyl)piperidin-4-yl)-3,9-diazaspiro[5.5]undecan-3-yl)pyrimidin-4-yl)methoxy)phenyl)propan-2-yl)benzonitrile (Compound 100) trifluoroacetate

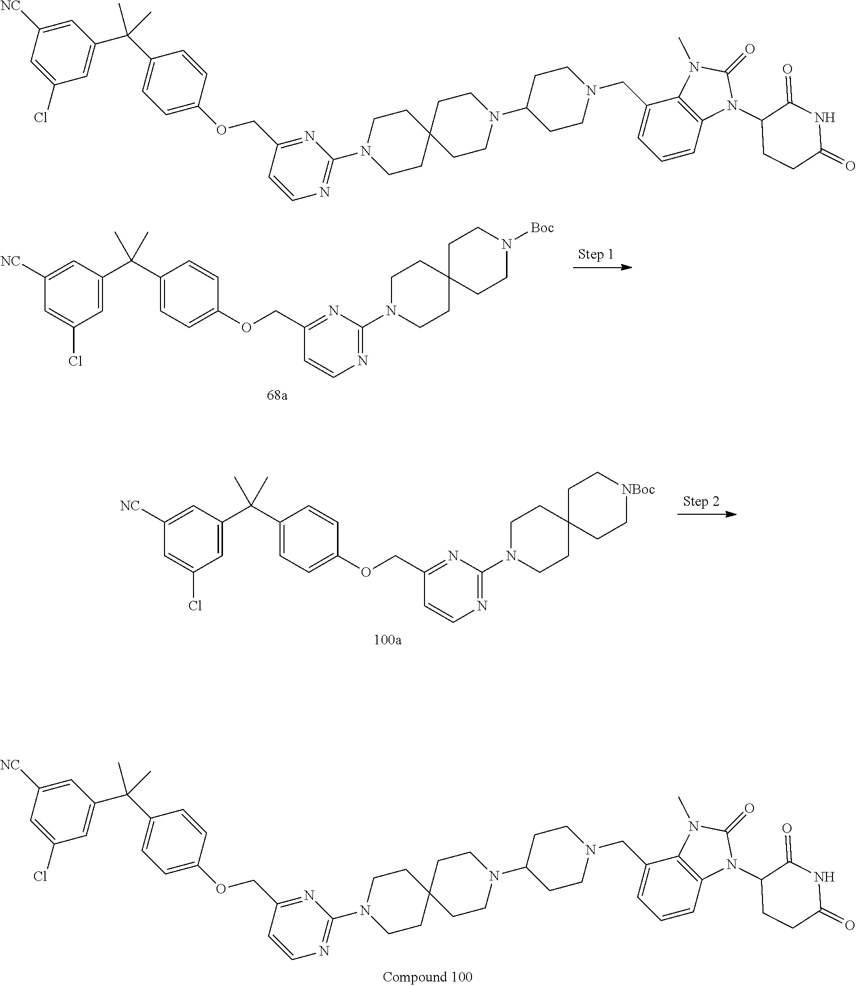 [SMILES: Cn1c(=O)n(C2CCC(=O)NC2=O)c2cccc(CN3CCC(N4CCC5(CCN(c6nccc(COc7ccc(C(C)(C)c8cc(Cl)cc(C#N)c8)cc7)n6)CC5)CC4)CC3)c21 ; CC(C)(C)OC(=O)N1CCC2(CC1)CCN(c1nccc(COc3ccc(C(C)(C)c4cc(Cl)cc(C#N)c4)cc3)n1)CC2 ; CC(C)(C)OC(=O)N1CCC2(CC1)CCN(c1nccc(COc3ccc(C(C)(C)c4cc(Cl)cc(C#N)c4)cc3)n1)CC2 ; Cn1c(=O)n(C2CCC(=O)NC2=O)c2cccc(CN3CCC(N4CCC5(CCN(c6nccc(COc7ccc(C(C)(C)c8cc(Cl)cc(C#N)c8)cc7)n6)CC5)CC4)CC3)c21]

Step 1: tert-butyl 4-(9-(4-((4-(2-(3-chloro-5-cyanophenyl)propan-2-yl)phenoxy)methyl)pyrimidin-2-yl)-3,9-diazaspiro[5.5]undecan-3-yl)piperidine-1-carboxylate (100a)

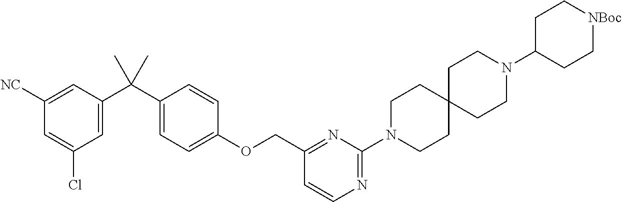 [SMILES: CC(C)(C)OC(=O)N1CCC(N2CCC3(CCN(c4nccc(COc5ccc(C(C)(C)c6cc(Cl)cc(C#N)c6)cc5)n4)CC3)CC2)CC1]

68a (400 mg, 0.65 mmol) was dissolved in 6 mL of dichloromethane, and 4 mL of trifluoroacetic acid was added. The mixture was reacted at room temperature for 2 h. The reaction system was concentrated under reduced pressure, and 10 mL of tetrahydrofuran and solid sodium bicarbonate (160 mg, 1.90 mmol) were added. The mixture was filtered by suction, and the filtrate was concentrated under reduced pressure. The residue was dissolved in 10 mL of 1,2-dichloroethane, and tert-butyl 4-oxopiperidine-1-carboxylate (120 mg, 0.6 mmol) was added. The mixture was reacted at room temperature for 1 h, and then sodium triacetoxyborohydride (130 mg, 0.6 mmol) was added. The resulting mixture was reacted at room temperature for 12 h. To the reaction liquid was added 30 mL of dichloromethane, and the mixture was adjusted to pH 9.0 with a saturated aqueous sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 100a (190 mg, yield: 42%).

LCMS m/z=699.3 [M+1]⁺

### Step 2: 3-chloro-5-(2-(4-((2-(9-(1-((1-(2,6-dioxopiperidin-3-yl)-3-methyl-2-oxo-2,3-dihydro-1H-benzo[d]imidazol-4-yl)methyl)piperidin-4-yl)-3,9-diazaspiro[5.5]undecan-3-yl)pyrimidin-4-yl)methoxy)phenyl)propan-2-yl)benzonitrile (Compound 100) trifluoroacetate

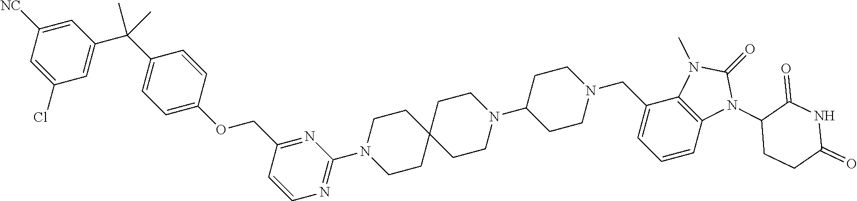 [SMILES: Cn1c(=O)n(C2CCC(=O)NC2=O)c2cccc(CN3CCC(N4CCC5(CCN(c6nccc(COc7ccc(C(C)(C)c8cc(Cl)cc(C#N)c8)cc7)n6)CC5)CC4)CC3)c21]

100a (190 mg, 0.27 mmol) was dissolved in 6 mL of dichloromethane, and 2 mL of trifluoroacetic acid was added. The mixture was reacted at room temperature for 2 h. The reaction liquid was concentrated under reduced pressure and dissolved in 30 mL of dichloromethane. The system was adjusted to pH 9.0 with a saturated aqueous sodium bicarbonate solution, and the organic phase was separated, dried over anhydrous sodium sulfate, and concentrated under reduced pressure, to afford a crude product. The above crude was dissolved in 10 mL of 1,2-dichloroethane, and 1-(2,6-dioxopiperidin-3-yl)-3-methyl-2-oxo-2,3-dihydro-1H-benzo[d]imidazole-4-carbaldehyde (see WO 2020113233 for the synthetic method) (86 mg, 0.3 mmol) was added. The mixture was reacted at room temperature for 1 h, and then sodium triacetoxyborohydride (130 mg, 0.61 mmol) was added. The resulting mixture was reacted at room temperature for 12 h. To the reaction liquid was added 30 mL of dichloromethane, and the mixture was adjusted to pH 9.0 with a saturated sodium bicarbonate solution. The organic phase was separated, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 m filter membrane, to prepare into a sample liquid. Mobile phase system: acetonitrile/water (containing 0.1% TFA). Gradient elution method: purification by gradient elution with acetonitrile from 5% to 60% (elution time: 15 min), lyophilization was performed to obtain compound 100 trifluoroacetate (110 mg).

¹H NMR (400 MHz, DMSO-d₆) δ 11.11 (s, 1H), 8.35 (d, 1H), 7.86-7.82 (m, 1H), 7.67-7.62 (m, 1H), 7.55-7.49 (m, 1H), 7.31-7.06 (m, 5H), 7.00-6.88 (m, 2H), 6.67 (d, 1H), 5.43 (dd, 1H), 4.97 (s, 2H), 4.70-4.20 (m, 2H), 3.82-3.69 (m, 4H), 3.63 (s, 3H), 3.40-3.23 (m, 4H), 3.20-3.00 (m, 3H), 2.99-2.59 (m, 5H), 2.36-2.15 (m, 2H), 2.10-1.75 (m, 5H), 1.74-1.50 (m, 10H), 1.46-1.30 (m, 2H).

LCMS m/z=435.9 [M/2+1]⁺

### Example 101

### 3-chloro-5-(2-(4-((2-(9-(7-((1-(2,6-dioxopiperidin-3-yl)-3-methyl-2-oxo-2,3-dihydro-1H-benzo[d]imidazol-4-yl)methyl)-7-azaspiro[3.5]nonan-2-yl)-3,9-diazaspiro[5.5]undecan-3-yl)pyrimidin-4-yl)methoxy)phenyl)propan-2-yl)benzonitrile (Compound 101) trifluoroacetate

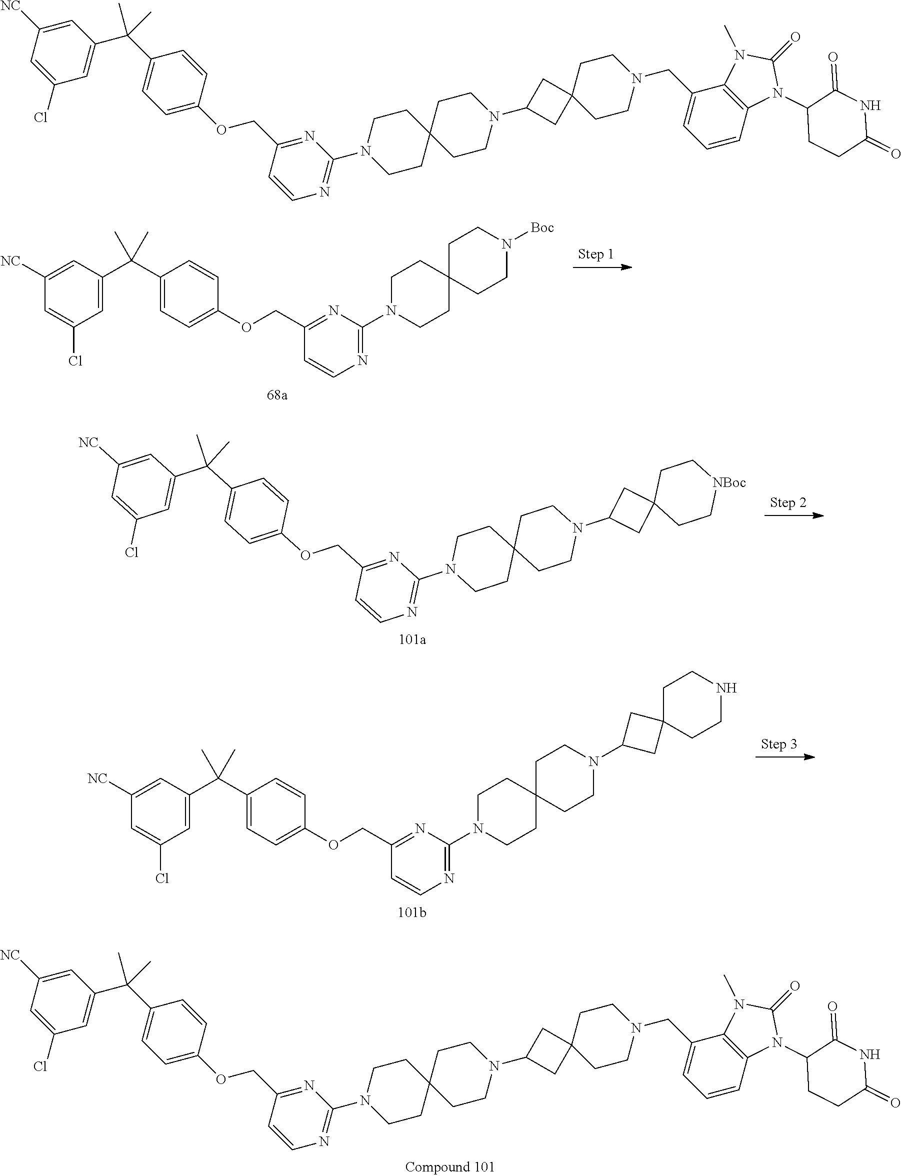

### Step 1: tert-butyl 2-(9-(4-((4-(2-(3-chloro-5-cyanophenyl)propan-2-yl)phenoxy)methyl)pyrimidin-2-yl)-3,9-diazaspiro[5.5]undecan-3-yl)-7-azaspiro[3.5]nonane-7-carboxylate (101a)

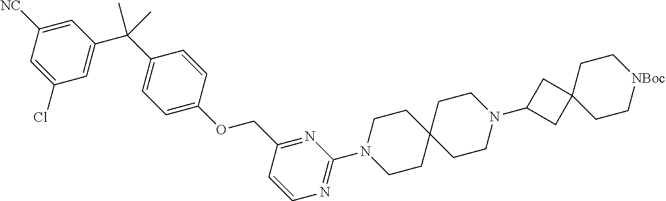 [SMILES: CC(C)(C)OC(=O)N1CCC2(CC1)CC(N1CCC3(CCN(c4nccc(COc5ccc(C(C)(C)c6cc(Cl)cc(C#N)c6)cc5)n4)CC3)CC1)C2]

Tert-butyl 9-(4-((4-(2-(3-chloro-5-cyanophenyl)propan-2-yl)phenoxy)methyl)pyrimidin-2-yl)-3,9-diazaspiro[5.5]undecane-3-carboxylate (68a) (400 mg, 0.65 mmol) was dissolved in 6 mL of dichloromethane, and 4 mL of trifluoroacetic acid was added. The mixture was reacted at room temperature for 2 h. The reaction system was concentrated under reduced pressure, and 10 mL of tetrahydrofuran and solid sodium bicarbonate (160 mg, 1.90 mmol) were added. The mixture was filtered by suction, and the filtrate was concentrated under reduced pressure. The residue was dissolved in 10 mL of 1,2-dichloroethane, and tert-butyl 2-oxo-7-azaspiro[3.5]nonane-7-carboxylate (290 mg, 1.2 mmol) was added. The mixture was reacted at room temperature for 1 h, and then sodium triacetoxyborohydride (251 mg, 1.2 mmol) was added. The resulting mixture was reacted at room temperature for 12 h. To the reaction liquid was added 30 mL of dichloromethane, and the mixture was adjusted to pH 9.0 with a saturated aqueous sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 101a (310 mg, yield: 65%).

LCMS m/z=370.4 [M/2+1]⁺

### Step 2: 3-(2-(4-((2-(9-(7-azaspiro[3.5]nonan-2-yl)-3,9-diazaspiro[5.5]undecan-3-yl)pyrimidin-4-yl)methoxy)phenyl)propan-2-yl)-5-chlorobenzonitrile (101b)

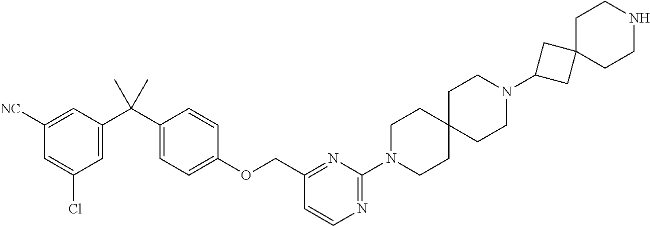 [SMILES: CC(C)(c1ccc(OCc2ccnc(N3CCC4(CC3)CCN(C3CC5(CCNCC5)C3)CC4)n2)cc1)c1cc(Cl)cc(C#N)c1]

101a (310 mg, 0.42 mmol) was dissolved in 6 mL of dichloromethane, and 2 mL of trifluoroacetic acid was added. The mixture was reacted at room temperature for 2 h. The reaction liquid was concentrated under reduced pressure and dissolved in 30 mL of dichloromethane. The mixture was adjusted to pH 9.0 with a saturated aqueous sodium bicarbonate solution, and the organic phase was separated, dried over anhydrous sodium sulfate, and concentrated under reduced pressure, to afford crude 101b (250 mg).

LCMS m/z=639.3 [M+1]⁺

### Step 3: 3-chloro-5-(2-(4-((2-(9-(7-((1-(2,6-dioxopiperidin-3-yl)-3-methyl-2-oxo-2,3-dihydro-1H-benzo[d]imidazol-4-yl)methyl)-7-azaspiro[3.5]nonan-2-yl)-3,9-diazaspiro[5.5]undecan-3-yl)pyrimidin-4-yl)methoxy)phenyl)propan-2-yl)benzonitrile (Compound 101) trifluoroacetate

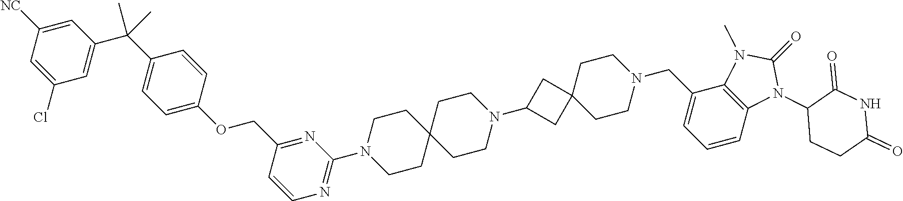 [SMILES: Cn1c(=O)n(C2CCC(=O)NC2=O)c2cccc(CN3CCC4(CC3)CC(N3CCC5(CCN(c6nccc(COc7ccc(C(C)(C)c8cc(Cl)cc(C#N)c8)cc7)n6)CC5)CC3)C4)c21]

The above crude 101b (150 mg) was dissolved in 10 mL of 1,2-dichloroethane, and 1-(2,6-dioxopiperidin-3-yl)-3-methyl-2-oxo-2,3-dihydro-1H-benzo[d]imidazole-4-carbaldehyde (see WO 2020113233 for the synthetic method) (72 mg, 0.25 mmol) was added. The mixture was reacted at room temperature for 1 h, and then sodium triacetoxyborohydride (63 mg, 0.3 mmol) was added. The resulting mixture was reacted at room temperature for 12 h. To the reaction liquid was added 30 mL of dichloromethane, and the mixture was adjusted to pH 9.0 with a saturated aqueous sodium bicarbonate solution. The organic phase was separated, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: purification by gradient elution with acetonitrile from 5% to 60% (elution time: 15 min), lyophilization was performed to obtain compound 101 trifluoroacetate (116 mg).

¹H NMR (400 MHz, DMSO-d₆) δ 11.12 (s, 1H), 8.35 (d, 1H), 7.88-7.80 (m, 1H), 7.70-7.60 (m, 1H), 7.56-7.48 (m, 1H), 7.33-7.08 (m, 5H), 6.98-6.90 (m, 2H), 6.68 (d, 1H), 5.44 (dd, 1H), 4.97 (s, 2H), 4.70-4.50 (m, 2H), 3.88-3.68 (m, 6H), 3.61 (s, 3H), 3.30-2.82 (m, 8H), 2.80-2.58 (m, 2H), 2.20-1.84 (m, 8H), 1.84-1.69 (m, 3H), 1.69-1.45 (m, 10H), 1.45-1.34 (m, 2H).

LCMS m/z=455.9 [M/2+1]⁺

### Example 102

### 3-chloro-5-(2-(4-((2-(4-(((1-(1-(2,6-dioxopiperidin-3-yl)-3-methyl-2-oxo-2,3-dihydro-1H-benzo[d]imidazol-5-yl)piperidin-4-yl)(methyl)amino)methyl)piperidin-1-yl)pyrimidin-4-yl)methoxy)phenyl)propan-2-yl)-2-ethoxybenzonitrile (Compound 102) trifluoroacetate

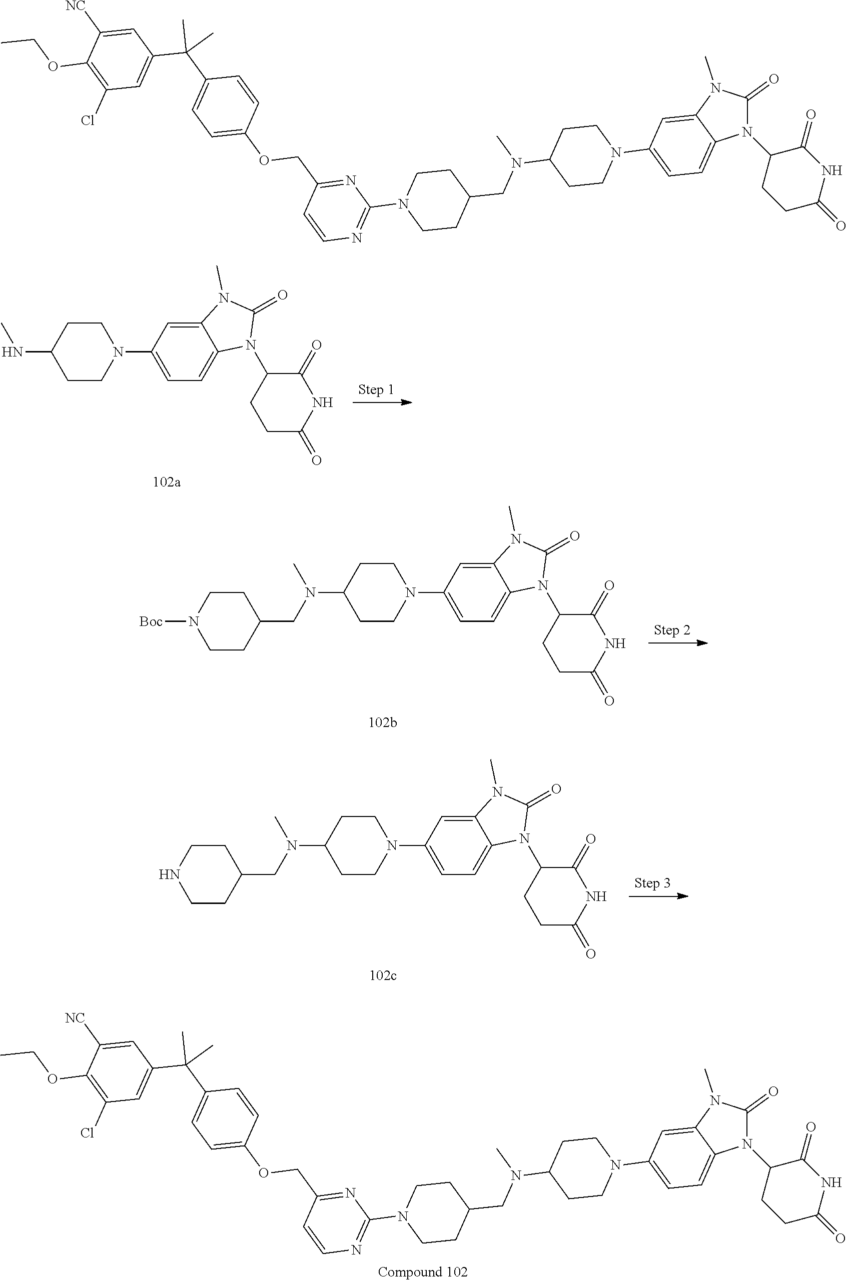 [SMILES: CCOc1c(Cl)cc(C(C)(C)c2ccc(OCc3ccnc(N4CCC(CN(C)C5CCN(c6ccc7c(c6)n(C)c(=O)n7C6CCC(=O)NC6=O)CC5)CC4)n3)cc2)cc1C#N ; CNC1CCN(c2ccc3c(c2)n(C)c(=O)n3C2CCC(=O)NC2=O)CC1 ; CN(CC1CCN(C(=O)OC(C)(C)C)CC1)C1CCN(c2ccc3c(c2)n(C)c(=O)n3C2CCC(=O)NC2=O)CC1 ; CN(CC1CCNCC1)C1CCN(c2ccc3c(c2)n(C)c(=O)n3C2CCC(=O)NC2=O)CC1 ; CCOc1c(Cl)cc(C(C)(C)c2ccc(OCc3ccnc(N4CCC(CN(C)C5CCN(c6ccc7c(c6)n(C)c(=O)n7C6CCC(=O)NC6=O)CC5)CC4)n3)cc2)cc1C#N]

### Step 1: tert-butyl 4-(((1-(1-(2,6-dioxopiperidin-3-yl)-3-methyl-2-oxo-2,3-dihydro-1H-benzo[d]imidazol-5-yl)piperidin-4-yl)(methyl)amino)methyl)piperidine-1-carboxylate (102b)

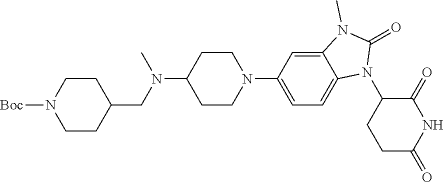 [SMILES: CN(CC1CCN(C(=O)OC(C)(C)C)CC1)C1CCN(c2ccc3c(c2)n(C)c(=O)n3C2CCC(=O)NC2=O)CC1]

3-(3-methyl-5-(4-(methylamino)piperidin-1-yl)-2-oxo-2,3-dihydro-1H-benzo[d]imidazol-1-yl)piperidine-2,6-dione (102a) hydrochloride (see WO 2020264499 for the synthetic method) (2.0 g) was dissolved in 20 mL of tetrahydrofuran, and 2 mL of DMSO and solid sodium bicarbonate (1.26 g, 15 mmol) were added. The mixture was stirred at room temperature for 0.5 h and then filtered by suction. To the filtrate were respectively added tert-butyl 4-formylpiperidine-1-carboxylate (2.13 g, 10 mmol) and glacial acetic acid (0.5 mL). The mixture was reacted at room temperature for 0.5 h, and then sodium triacetoxyborohydride (2.12 g, 10 mmol) was added. The resulting mixture was reacted at room temperature for 16 h. To the reaction liquid was added 10 mL of aqueous sodium bicarbonate solution, and the mixture was extracted with ethyl acetate (30 mL×3). The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 102b (2.50 g).

LCMS m/z=569.3 [M+1]⁺

### Step 2: 3-(3-methyl-5-(4-(methyl(piperidin-4-ylmethyl)amino)piperidin-1-yl)-2-oxo-2,3-dihydro-1H-benzo[d]imidazol-1-yl)piperidine-2,6-dione (102c) trifluoroacetate

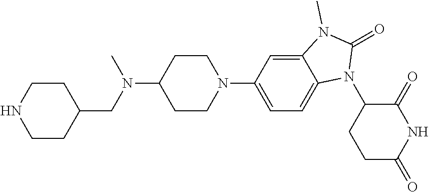 [SMILES: CN(CC1CCNCC1)C1CCN(c2ccc3c(c2)n(C)c(=O)n3C2CCC(=O)NC2=O)CC1]

To a reaction flask were successively added 102b (2.50 g, 4.4 mmol), trifluoroacetic acid (20 mL) and dichloromethane (20 mL), and the mixture was reacted at room temperature for 2 h. The reaction liquid was concentrated under reduced pressure, to afford crude 102c trifluoroacetate (2.50 g).

### Step 3: 3-chloro-5-(2-(4-((2-(4-(((1-(1-(2,6-dioxopiperidin-3-yl)-3-methyl-2-oxo-2,3-dihydro-1H-benzo[d]imidazol-5-yl)piperidin-4-yl)(methyl)amino)methyl)piperidin-1-yl)pyrimidin-4-yl)methoxy)phenyl)propan-2-yl)-2-ethoxybenzonitrile (Compound 102) trifluoroacetate

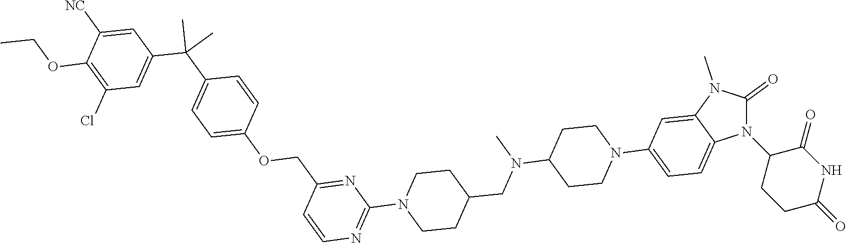 [SMILES: CCOc1c(Cl)cc(C(C)(C)c2ccc(OCc3ccnc(N4CCC(CN(C)C5CCN(c6ccc7c(c6)n(C)c(=O)n7C6CCC(=O)NC6=O)CC5)CC4)n3)cc2)cc1C#N]

The above crude 102c trifluoroacetate (2.50 g) was dissolved in 20 mL of DMF, and solid potassium bicarbonate (3.0 g, 30 mmol) was added. The mixture was stirred at room temperature for 0.5 h, and then 3-chloro-5-(2-(4-((2-chloropyrimidin-4-yl)methoxy)phenyl)propan-2-yl)-2-ethoxybenzonitrile (18g) (2.00 g, 4.52 mmol) was added. The resulting mixture was warmed to 80° C. and stirred for 4 h. The reaction liquid was cooled to room temperature, and 20 mL of water was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 102 trifluoroacetate (0.6 g).

¹H NMR (400 MHz, DMSO-d₆) δ 11.06 (s, 1H), 8.36 (d, 1H), 7.63-7.52 (m, 2H), 7.23-7.13 (m, 2H), 7.04-6.86 (m, 4H), 6.76-6.65 (m, 2H), 5.30 (dd, 1H), 4.98 (s, 2H), 4.75-4.62 (m, 2H), 4.19 (q, 2H), 3.84-3.71 (m, 2H), 3.48-3.37 (m, 2H), 3.32 (s, 3H), 3.24-3.12 (m, 1H), 3.02-2.55 (m, 10H), 2.21-1.72 (m, 8H), 1.63 (s, 6H), 1.37 (t, 3H), 1.30-1.08 (m, 2H).

LCMS m/z=874.4 [M+1]⁺.

### Example 103

### 3-chloro-5-(2-(4-((2-(3-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)azetidin-1-yl)pyrimidin-4-yl)methoxy)phenyl)propan-2-yl)benzonitrile (Compound 103) trifluoroacetate

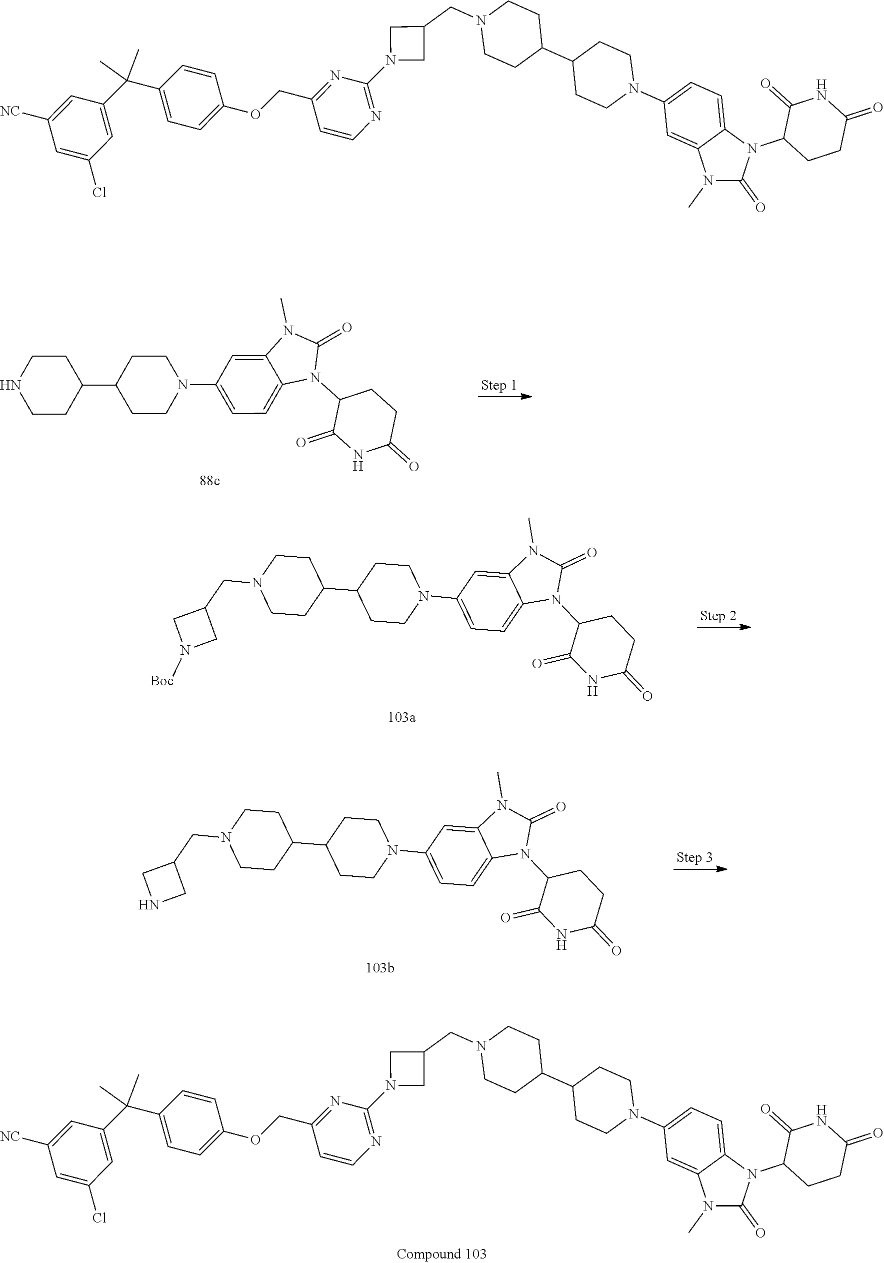 [SMILES: Cn1c(=O)n(C2CCC(=O)NC2=O)c2ccc(N3CCC(C4CCN(CC5CN(c6nccc(COc7ccc(C(C)(C)c8cc(Cl)cc(C#N)c8)cc7)n6)C5)CC4)CC3)cc21 ; Cn1c(=O)n(C2CCC(=O)NC2=O)c2ccc(N3CCC(C4CCNCC4)CC3)cc21 ; Cn1c(=O)n(C2CCC(=O)NC2=O)c2ccc(N3CCC(C4CCN(CC5CN(C(=O)OC(C)(C)C)C5)CC4)CC3)cc21 ; Cn1c(=O)n(C2CCC(=O)NC2=O)c2ccc(N3CCC(C4CCN(CC5CNC5)CC4)CC3)cc21 ; Cn1c(=O)n(C2CCC(=O)NC2=O)c2ccc(N3CCC(C4CCN(CC5CN(c6nccc(COc7ccc(C(C)(C)c8cc(Cl)cc(C#N)c8)cc7)n6)C5)CC4)CC3)cc21]

### Step 1: tert-butyl 3-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)azetidine-1-carboxylate (103a)

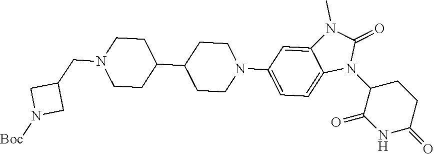 [SMILES: Cn1c(=O)n(C2CCC(=O)NC2=O)c2ccc(N3CCC(C4CCN(CC5CN(C(=O)OC(C)(C)C)C5)CC4)CC3)cc21]

The above crude 3-(5-([4,4′-bipiperidin]-1-yl)-3-methyl-2-oxo-2,3-dihydro-1H-benzo[d]imidazol-1-yl)piperidine-2,6-dione (88c) trifluoroacetate (210 mg) was dissolved in 25 mL of THF, and solid sodium bicarbonate (86 mg, 1.02 mmol) was added. The mixture was stirred at room temperature for 20 min, and then tert-butyl 4-formylazetidine-1-carboxylate (127 mg, 0.69 mmol) and 0.1 mL of acetic acid were added. The mixture was stirred at room temperature for 60 min, and then sodium triacetoxyborohydride (145 mg, 0.68 mmol) was added. The resulting mixture was reacted at room temperature for 16 h. To the reaction system was slowly added 40 mL of saturated aqueous sodium bicarbonate solution, and the mixture was extracted with 100 mL of DCM. The organic phase was washed with 80 mL of water, dried over anhydrous sodium sulfate, and concentrated under reduced pressure. The crude product was separated and purified by silica gel column chromatography (dichloromethane/methanol (v/v)=15:1), to afford 103a (180 mg, two-step yield from compound 88b: 91%).

LCMS m/z=595.3 [M+1]⁺

### Step 2: 3-(5-(1′-(azetidin-3-ylmethyl)-[4,4′-bipiperidin]-1-yl)-3-methyl-2-oxo-2,3-dihydro-1H-benzo[d]imidazol-1-yl)piperidine-2,6-dione (103b) trifluoroacetate

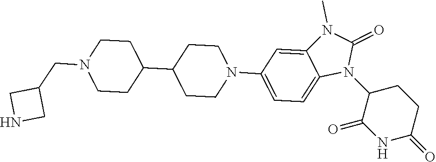 [SMILES: Cn1c(=O)n(C2CCC(=O)NC2=O)c2ccc(N3CCC(C4CCN(CC5CNC5)CC4)CC3)cc21]

103a (180 mg, 0.30 mmol) was dissolved in 10 mL of DCM, and 4 mL of trifluoroacetic acid was added. The mixture was stirred at room temperature for 2 h. The reaction liquid was concentrated under reduced pressure, to afford crude 103b trifluoroacetate (200 mg).

LCMS m/z=495.3 [M+1]⁺

### Step 3: 3-chloro-5-(2-(4-((2-(3-((1′-(1-(2,6-dioxopiperidin-3-yl)-3-methyl-2-oxo-2,3-dihydro-1H-benzo[d]imidazol-5-yl)-[4,4′-bipiperidin]-1-yl)methyl)azetidin-1-yl)pyrimidin-4-yl)methoxy)phenyl)propan-2-yl)benzonitrile (Compound 103) trifluoroacetate

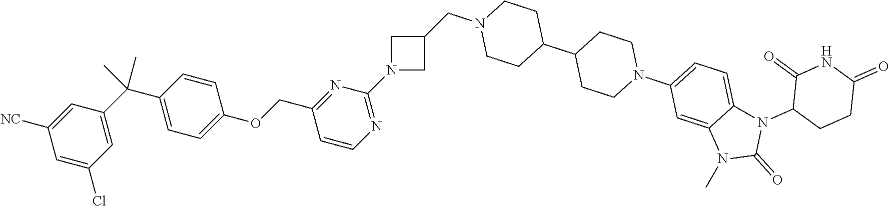 [SMILES: Cn1c(=O)n(C2CCC(=O)NC2=O)c2ccc(N3CCC(C4CCN(CC5CN(c6nccc(COc7ccc(C(C)(C)c8cc(Cl)cc(C#N)c8)cc7)n6)C5)CC4)CC3)cc21]

3-chloro-5-(2-(4-((2-chloropyrimidin-4-yl)methoxy)phenyl)propan-2-yl)benzonitrile (9d) (162 mg, 0.40 mmol) was dissolved in 6 ml of DMF, and the above crude 103b trifluoroacetate (200 mg) and solid potassium bicarbonate (265 mg, 2.65 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 103 trifluoroacetate (70 mg).

¹H NMR (400 MHz, DMSO-d₆) δ 11.11 (s, 1H), 8.37 (d, 1H), 7.88-7.80 (m, 1H), 7.69-7.61 (m, 1H), 7.56-7.50 (m, 1H), 7.48-7.32 (m, 1H), 7.26-7.10 (m, 4H), 7.00-6.89 (m, 2H), 6.80 (d, 1H), 5.39 (dd, 1H), 4.98 (s, 2H), 4.30-4.14 (m, 2H), 3.96-3.82 (m, 2H), 3.75-3.60 (m, 2H), 3.50-3.40 (m, 4H), 3.40-3.30 (m, 5H), 3.28-3.13 (m, 1H), 3.03-2.83 (m, 3H), 2.80-2.58 (m, 2H), 2.10-1.86 (m, 5H), 1.72-1.36 (m, 12H).

LCMS m/z=856.4 [M+1]⁺

### Example 104

### 3-chloro-5-(2-(4-((2-(1″-(1-(2,6-dioxopiperidin-3-yl)-3-methyl-2-oxo-2,3-dihydro-1H-benzo[d]imidazol-5-yl)-[4,1′:4′,4″-terpiperidin]-1-yl)pyrimidin-4-yl)methoxy)phenyl) propan-2-yl)benzonitrile (compound 104) trifluoroacetate

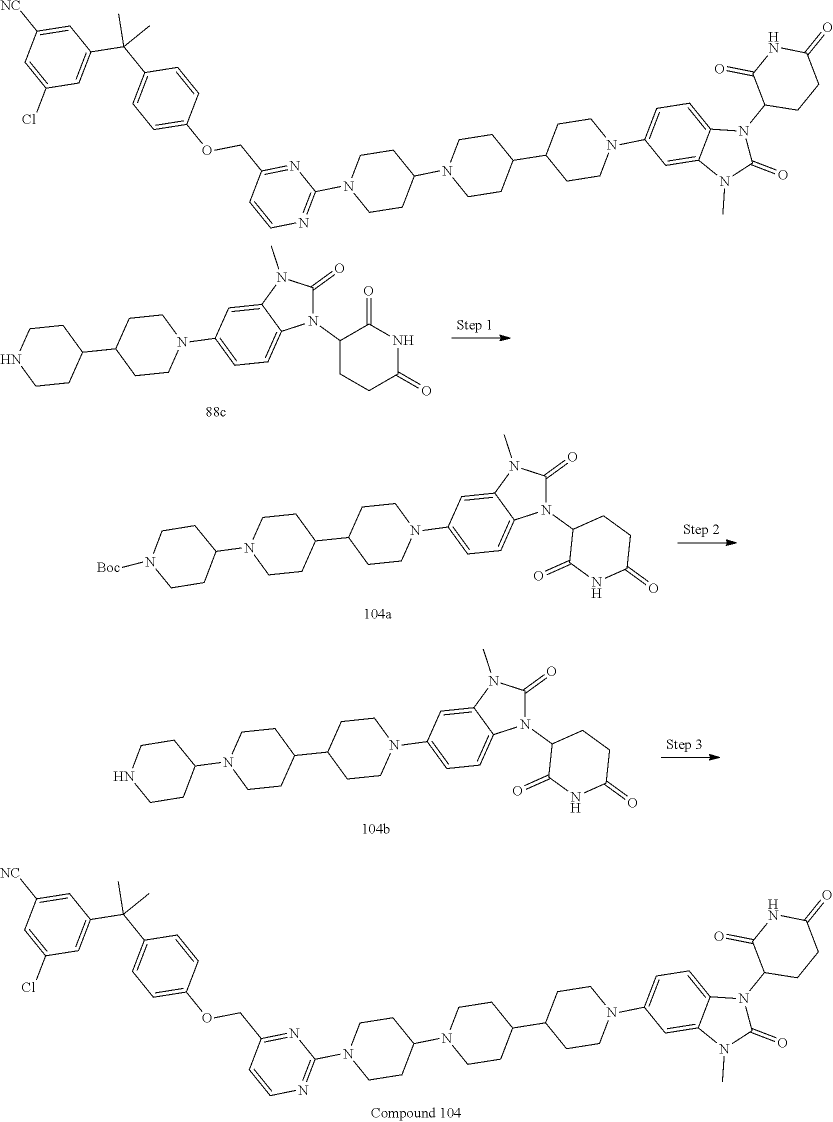

### Step 1: tert-butyl 1″-(1-(2,6-dioxopiperidin-3-yl)-3-methyl-2-oxo-2,3-dihydro-1H-benzo[d]imidazol-5-yl)-[4,1′:4′,4″-terpiperidine]-1-carboxylate (104a)

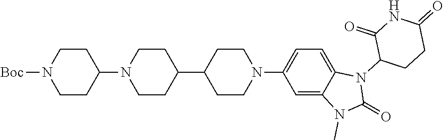 [SMILES: Cn1c(=O)n(C2CCC(=O)NC2=O)c2ccc(N3CCC(C4CCN(C5CCN(C(=O)OC(C)(C)C)CC5)CC4)CC3)cc21]

The above crude 3-(5-([4,4′-bipiperidin]-1-yl)-3-methyl-2-oxo-2,3-dihydro-1H-benzo[d]imidazol-1-yl)piperidine-2,6-dione (88c) trifluoroacetate (210 mg) was dissolved in 25 mL of THF, and solid sodium bicarbonate (86 mg, 1.02 mmol) was added. The mixture was stirred at room temperature for 20 min, and then N-Boc-4-piperidone (127 mg, 0.64 mmol) and 0.1 mL of acetic acid were added. The mixture was warmed to 50° C., stirred for 60 min, and then cooled to room temperature. Sodium triacetoxyborohydride (145 mg, 0.68 mmol) was added, and the resulting mixture was reacted at room temperature for 16 h. To the reaction system was slowly added 40 mL of saturated aqueous sodium bicarbonate solution, and the mixture was extracted with 100 mL of DCM. The organic phase was washed with 80 mL of water, dried over anhydrous sodium sulfate, and concentrated under reduced pressure. The crude product was separated and purified by silica gel column chromatography (dichloromethane/methanol (v/v)=15:1), to afford 104a (65 mg, two-step yield from compound 88b: 32%).

LCMS m/z=609.3 [M+1]⁺

### Step 2: 3-(5-([4,1′:4′,4″-terpiperidin]-1″-yl)-3-methyl-2-oxo-2,3-dihydro-1H-benzo[d]imidazol-1-yl)piperidine-2,6-dione (104b) trifluoroacetate

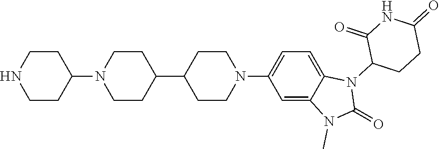 [SMILES: Cn1c(=O)n(C2CCC(=O)NC2=O)c2ccc(N3CCC(C4CCN(C5CCNCC5)CC4)CC3)cc21]

104a (65 mg, 0.11 mmol) was dissolved in 10 mL of DCM, and 4 mL of trifluoroacetic acid was added. The mixture was stirred at room temperature for 2 h. The reaction liquid was concentrated under reduced pressure, to afford crude 104b trifluoroacetate (80 mg).

LCMS m/z=509.3 [M+1]⁺

### Step 3: 3-chloro-5-(2-(4-((2-(1″-(1-(2,6-dioxopiperidin-3-yl)-3-methyl-2-oxo-2,3-dihydro-1H-benzo[d]imidazol-5-yl)-[4,1′:4′,4″-terpiperidin]-1-yl)pyrimidin-4-yl)methoxy)phenyl) propan-2-yl)benzonitrile (Compound 104) trifluoroacetate

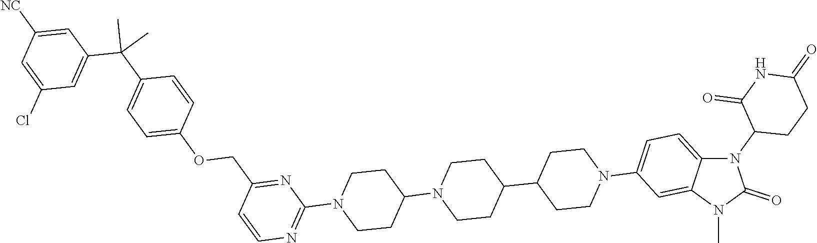 [SMILES: Cn1c(=O)n(C2CCC(=O)NC2=O)c2ccc(N3CCC(C4CCN(C5CCN(c6nccc(COc7ccc(C(C)(C)c8cc(Cl)cc(C#N)c8)cc7)n6)CC5)CC4)CC3)cc21]

3-chloro-5-(2-(4-((2-chloropyrimidin-4-yl)methoxy)phenyl)propan-2-yl)benzonitrile (9d) (62 mg, 0.15 mmol) was dissolved in 6 mL of DMF, and the above crude 104b trifluoroacetate (80 mg) and solid potassium bicarbonate (72 mg, 0.72 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 104 trifluoroacetate (15 mg).

¹H NMR (400 MHz, DMSO-d₆) δ 11.09 (s, 1H), 8.45-8.35 (m, 1H), 7.88-7.81 (m, 1H), 7.67-7.62 (m, 1H), 7.56-7.50 (m, 1H), 7.45-6.89 (m, 7H), 6.80-6.70 (m, 1H), 5.45-5.30 (m, 1H), 4.99 (s, 2H), 4.90-4.75 (m, 2H), 4.30-3.73 (m, 2H), 3.70-3.58 (m, 2H), 3.58-3.42 (m, 3H), 3.35 (s, 3H), 3.30-3.05 (m, 2H), 3.05-2.80 (m, 5H), 2.79-2.56 (m, 2H), 2.20-1.77 (m, 7H), 1.70-1.27 (m, 12H).

LCMS m/z=870.4 [M+1]⁺

### Example 105

### 3-chloro-5-(2-(4-((2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)pyrimidin-4-yl)methoxy)phenyl)propan-2-yl)benzonitrile (Compound 105) trifluoroacetate

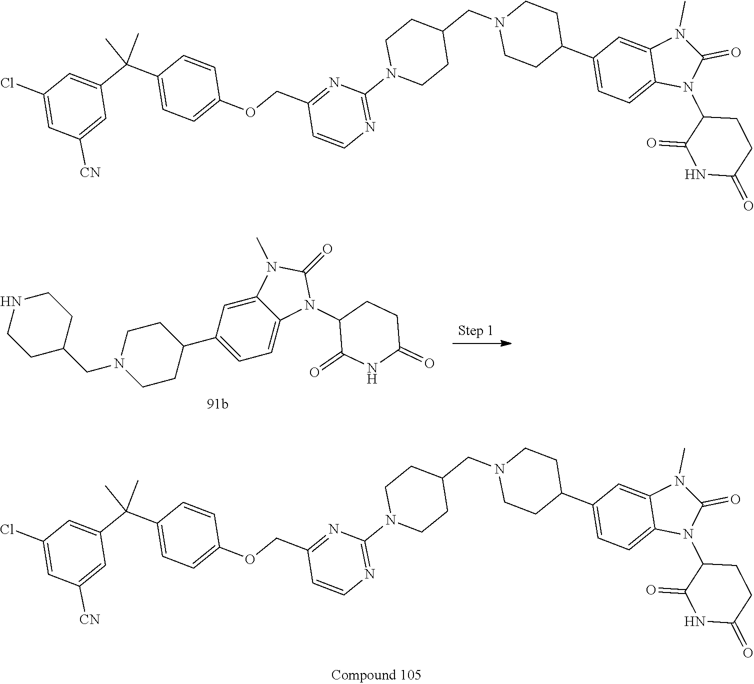 [SMILES: Cn1c(=O)n(C2CCC(=O)NC2=O)c2ccc(C3CCN(CC4CCN(c5nccc(COc6ccc(C(C)(C)c7cc(Cl)cc(C#N)c7)cc6)n5)CC4)CC3)cc21 ; Cn1c(=O)n(C2CCC(=O)NC2=O)c2ccc(C3CCN(CC4CCNCC4)CC3)cc21 ; Cn1c(=O)n(C2CCC(=O)NC2=O)c2ccc(C3CCN(CC4CCN(c5nccc(COc6ccc(C(C)(C)c7cc(Cl)cc(C#N)c7)cc6)n5)CC4)CC3)cc21]

3-chloro-5-(2-(4-((2-chloropyrimidin-4-yl)methoxy)phenyl)propan-2-yl)benzonitrile (9d) (166 mg, 0.40 mmol) was dissolved in 6 mL of DMF, and the above crude 91b trifluoroacetate (170 mg) and solid potassium bicarbonate (140 mg, 1.40 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 105 trifluoroacetate (80 mg).

¹H NMR (400 MHz, DMSO-d₆) δ 11.09 (s, 1H), 8.37 (d, 1H), 7.87-7.81 (m, 1H), 7.68-7.62 (m, 1H), 7.56-7.50 (m, 1H), 7.23-7.13 (m, 2H), 7.12-7.02 (m, 2H), 7.00-6.90 (m, 3H), 6.70 (d, 1H), 5.36 (dd, 1H), 4.99 (s, 2H), 4.78-4.59 (m, 2H), 3.71-3.58 (m, 2H), 3.42-3.31 (m, 3H), 3.20-2.82 (m, 8H), 2.80-2.56 (m, 2H), 2.27-1.79 (m, 8H), 1.64 (s, 6H), 1.28-1.10 (m, 2H).

LCMS m/z=801.3 [M+1].

### Example 106

### 3-chloro-5-(2-(4-((2-(2-(4-(1-(2,6-dioxopiperidin-3-yl)-3-methyl-2-oxo-2,3-dihydro-1H-benzo[d]imidazol-5-yl)piperidin-1-yl)-7-azaspiro[3.5]nonan-7-yl)pyrimidin-4-yl)methoxy)phenyl)propan-2-yl)benzonitrile (Compound 106) trifluoroacetate

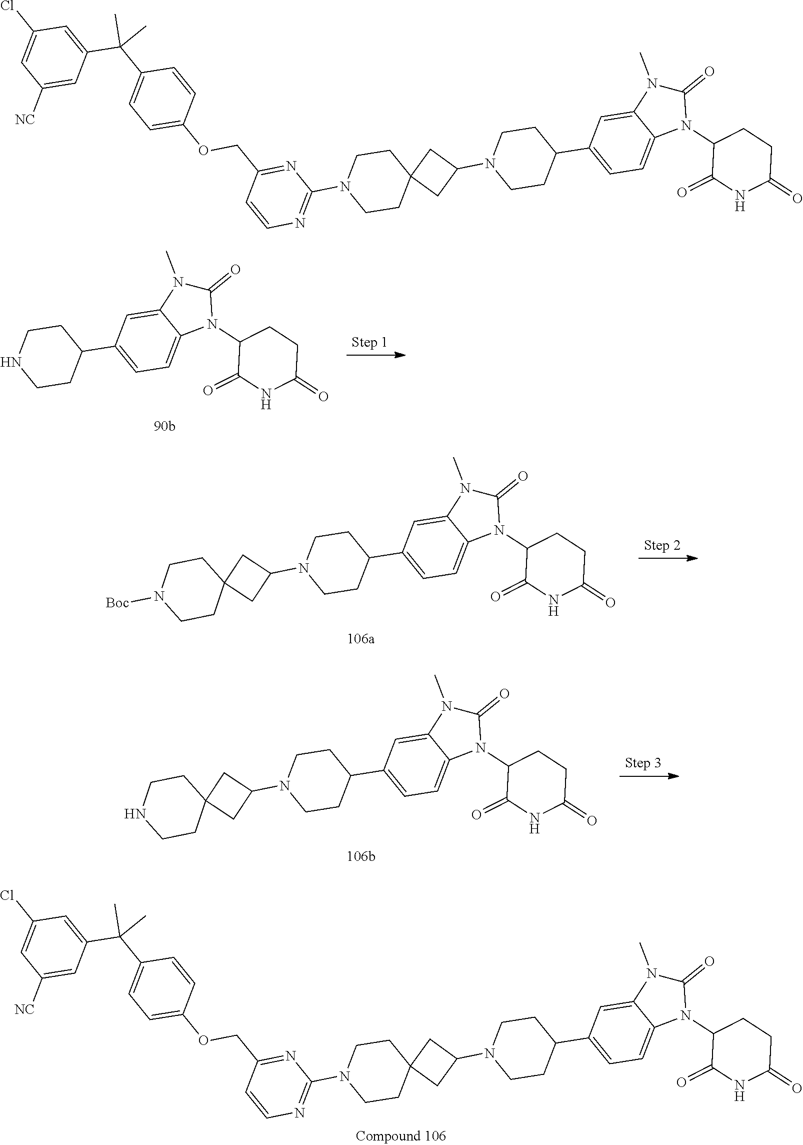 [SMILES: Cn1c(=O)n(C2CCC(=O)NC2=O)c2ccc(C3CCN(C4CC5(CCN(c6nccc(COc7ccc(C(C)(C)c8cc(Cl)cc(C#N)c8)cc7)n6)CC5)C4)CC3)cc21 ; Cn1c(=O)n(C2CCC(=O)NC2=O)c2ccc(C3CCNCC3)cc21 ; Cn1c(=O)n(C2CCC(=O)NC2=O)c2ccc(C3CCN(C4CC5(CCN(C(=O)OC(C)(C)C)CC5)C4)CC3)cc21 ; Cn1c(=O)n(C2CCC(=O)NC2=O)c2ccc(C3CCN(C4CC5(CCNCC5)C4)CC3)cc21 ; Cn1c(=O)n(C2CCC(=O)NC2=O)c2ccc(C3CCN(C4CC5(CCN(c6nccc(COc7ccc(C(C)(C)c8cc(Cl)cc(C#N)c8)cc7)n6)CC5)C4)CC3)cc21]

### Step 1: tert-buty 2-(4-(1-(2,6-dioxopiperidin-3-yl)-3-methyl-2-oxo-23-dihydro-1H-benzo[d]imidazol-5-yl)piperidin-1-yl)-7-azaspiro[3.5]nonane-7-carboxylate (106a)

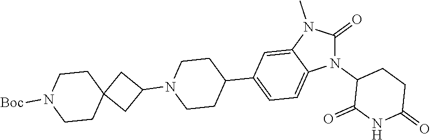 [SMILES: Cn1c(=O)n(C2CCC(=O)NC2=O)c2ccc(C3CCN(C4CC5(CCN(C(=O)OC(C)(C)C)CC5)C4)CC3)cc21]

The above crude 3-(3-methyl-2-oxo-5-(piperidin-4-yl)-2,3-dihydro-1H-benzo[d]imidazol-1-yl)piperidine-2,6-dione (90b) trifluoroacetate (250 mg) was dissolved in 15 mL of THF, and solid sodium bicarbonate (118 mg, 1.40 mmol) was added. The mixture was stirred at room temperature for 20 min, and then tert-butyl 2-oxo-7-azaspiro[3.5]nonane-7-carboxylate (205 mg, 0.86 mmol) and 0.1 mL of acetic acid were successively added. The mixture was warmed to 50° C., stirred for 2 h, and then cooled to room temperature. Sodium triacetoxyborohydride (191 mg, 0.90 mmol) was added, and the resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 106a (220 mg, two-step yield from compound 90a: 82%).

LCMS m/z=566.3 [M+1]⁺

### Step 2: 3-(5-(1-(7-azaspiro[3.5]nonan-2-yl)piperidin-4-yl)-3-methyl-2-oxo-2,3-dihydro-1H-benzo[d]imidazol-1-yl)piperidine-2,6-dione (106b) trifluoroacetate

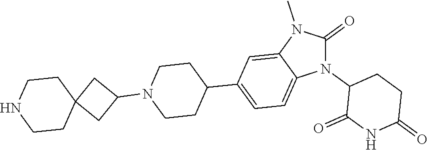 [SMILES: Cn1c(=O)n(C2CCC(=O)NC2=O)c2ccc(C3CCN(C4CC5(CCNCC5)C4)CC3)cc21]

106a (220 mg, 0.39 mmol) was dissolved in 10 mL of DCM, and 4 mL of trifluoroacetic acid was added. The mixture was stirred at room temperature for 2 h. The reaction liquid was concentrated under reduced pressure, to afford crude 106b trifluoroacetate (250 mg).

LCMS m/z=466.3 [M+1]⁺

### Step 3: 3-chloro-5-(2-(4-((2-(2-(4-(1-(2,6-dioxopiperidin-3-yl)-3-methyl-2-oxo-2,3-dihydro-1H-benzo[d]imidazol-5-yl)piperidin-1-yl)-7-azaspiro[3.5]nonan-7-yl)pyrimidin-4-yl)methoxy)phenyl)propan-2-yl)benzonitrile (Compound 106) trifluoroacetate

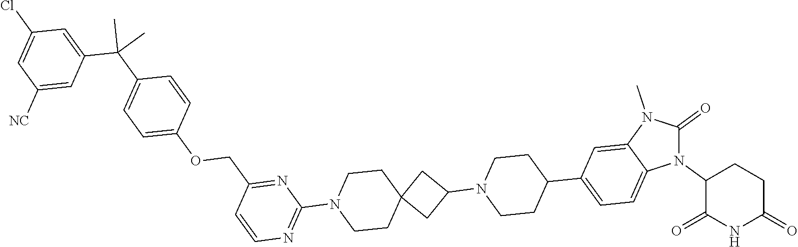 [SMILES: Cn1c(=O)n(C2CCC(=O)NC2=O)c2ccc(C3CCN(C4CC5(CCN(c6nccc(COc7ccc(C(C)(C)c8cc(Cl)cc(C#N)c8)cc7)n6)CC5)C4)CC3)cc21]

3-chloro-5-(2-(4-((2-chloropyrimidin-4-yl)methoxy)phenyl)propan-2-yl)benzonitrile (9d) (108 mg, 0.26 mmol) was dissolved in 6 mL of DMF, and the above crude 106b trifluoroacetate (125 mg) and solid potassium bicarbonate (108 mg, 1.08 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 106 trifluoroacetate (40 mg).

¹H NMR (400 MHz, DMSO-d₆) δ 11.08 (s, 1H), 8.35 (d, 1H), 7.88-7.81 (m, 1H), 7.69-7.62 (m, 1H), 7.56-7.50 (m, 1H), 7.22-7.13 (m, 2H), 7.13-7.00 (m, 2H), 7.00-6.87 (m, 3H), 6.68 (d, 1H), 5.36 (dd, 1H), 4.97 (s, 2H), 3.83-3.63 (m, 5H), 3.55-3.42 (m, 2H), 3.40-3.30 (m, 3H), 3.05-2.57 (m, 6H), 2.38-2.20 (m, 2H), 2.15-1.75 (m, 7H), 1.75-1.45 (m, 10H).

LCMS m/z=827.3 [M+1]⁺.

### Example 107

### 3-chloro-5-(2-(4-((2-(2-(4-(1-(2,6-dioxopiperidin-3-yl)-3-methyl-2-oxo-2,3-dihydro-1H-benzo[d]imidazol-5-yl)piperidin-1-yl)-7-azaspiro[3.5]nonan-7-yl)pyrimidin-4-yl)methoxy)phenyl)propan-2-yl)-2-ethoxybenzonitrile (Compound 107) trifluoroacetate

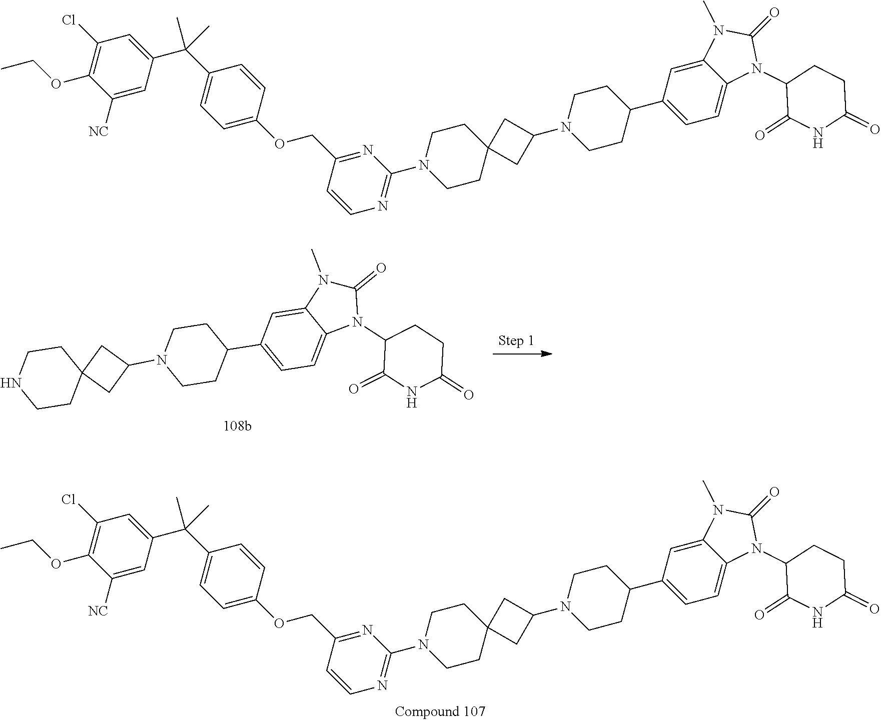 [SMILES: CCOc1c(Cl)cc(C(C)(C)c2ccc(OCc3ccnc(N4CCC5(CC4)CC(N4CCC(c6ccc7c(c6)n(C)c(=O)n7C6CCC(=O)NC6=O)CC4)C5)n3)cc2)cc1C#N ; Cn1c(=O)n(C2CCC(=O)NC2=O)c2ccc(C3CCN(C4CC5(CCNCC5)C4)CC3)cc21 ; CCOc1c(Cl)cc(C(C)(C)c2ccc(OCc3ccnc(N4CCC5(CC4)CC(N4CCC(c6ccc7c(c6)n(C)c(=O)n7C6CCC(=O)NC6=O)CC4)C5)n3)cc2)cc1C#N]

3-chloro-5-(2-(4-((2-chloropyrimidin-4-yl)methoxy)phenyl)propan-2-yl)-2-ethoxybenzonitrile (18g) (120 mg, 0.27 mmol) was dissolved in 6 mL of DMF, and the above crude 106b trifluoroacetate (125 mg) and solid potassium bicarbonate (108 mg, 1.08 mmol) were successively added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 107 trifluoroacetate (65 mg).

¹H NMR (400 MHz, DMSO-d₆) δ 11.08 (s, 1H), 8.38-8.31 (m, 1H), 7.63-7.53 (m, 2H), 7.23-6.85 (m, 7H), 6.71-6.66 (m, 1H), 5.42-5.30 (m, 1H), 4.97 (s, 2H), 4.20 (q, 2H), 3.90-3.62 (m, 5H), 3.54-3.44 (m, 2H), 3.40-3.32 (m, 3H), 3.03-2.80 (m, 4H), 2.80-2.57 (m, 2H), 2.40-2.15 (m, 2H), 2.15-1.75 (m, 7H), 1.74-1.50 (m, 10H), 1.37 (t, 3H).

LCMS m/z=871.3 [M+1]⁺

### Example 108

### 3-chloro-5-(2-(4-((2-(2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-7-azaspiro[3.5]nonan-7-yl)pyrimidin-4-yl)methoxy)phenyl)propan-2-yl)benzonitrile (Compound 108) trifluoroacetate

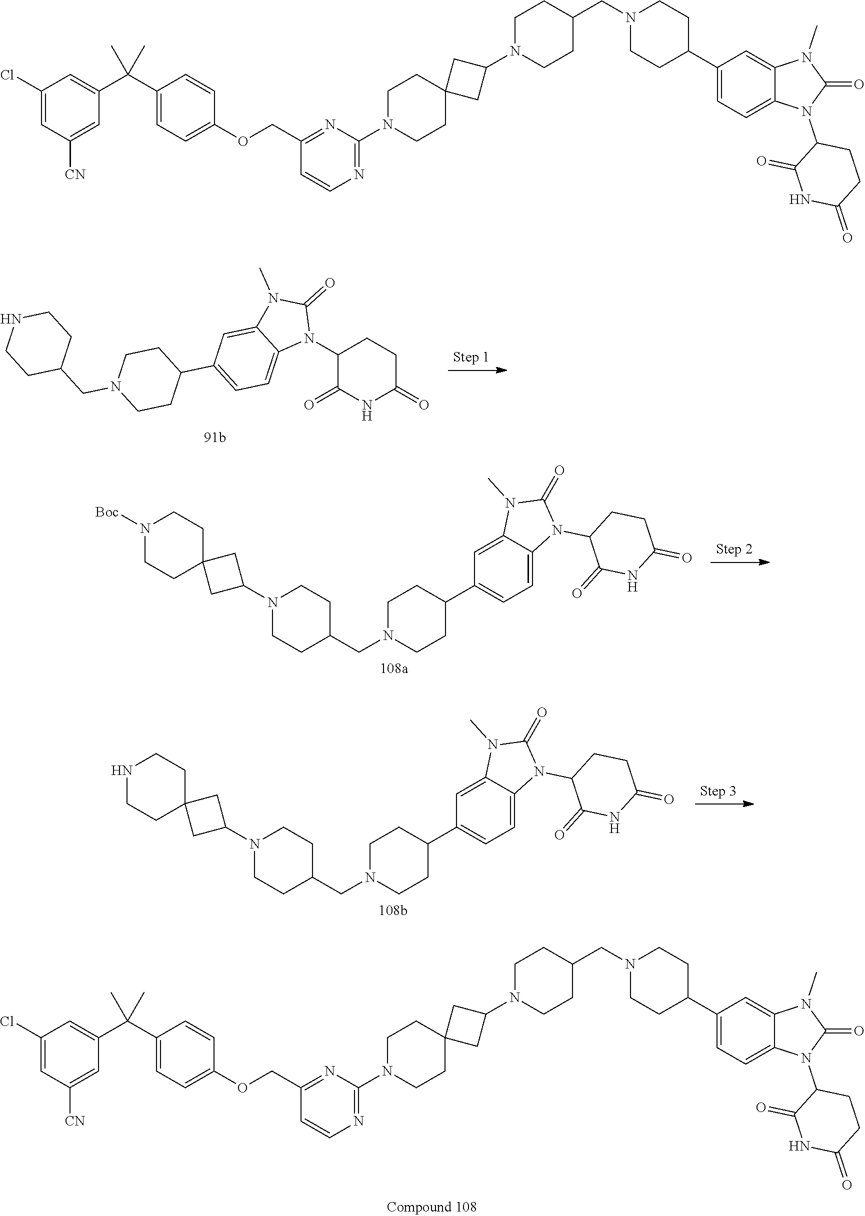 [SMILES: Cn1c(=O)n(C2CCC(=O)NC2=O)c2ccc(C3CCN(CC4CCN(C5CC6(CCN(c7nccc(COc8ccc(C(C)(C)c9cc(Cl)cc(C#N)c9)cc8)n7)CC6)C5)CC4)CC3)cc21 ; Cn1c(=O)n(C2CCC(=O)NC2=O)c2ccc(C3CCN(CC4CCNCC4)CC3)cc21 ; Cn1c(=O)n(C2CCC(=O)NC2=O)c2ccc(C3CCN(CC4CCN(C5CC6(CCN(C(=O)OC(C)(C)C)CC6)C5)CC4)CC3)cc21 ; Cn1c(=O)n(C2CCC(=O)NC2=O)c2ccc(C3CCN(CC4CCN(C5CC6(CCNCC6)C5)CC4)CC3)cc21 ; Cn1c(=O)n(C2CCC(=O)NC2=O)c2ccc(C3CCN(CC4CCN(C5CC6(CCN(c7nccc(COc8ccc(C(C)(C)c9cc(Cl)cc(C#N)c9)cc8)n7)CC6)C5)CC4)CC3)cc21]

### Step 1: tert-butyl 2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-7-azaspiro[3.5]nonane-7-carboxylate (108a)

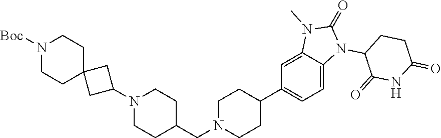 [SMILES: Cn1c(=O)n(C2CCC(=O)NC2=O)c2ccc(C3CCN(CC4CCN(C5CC6(CCN(C(=O)OC(C)(C)C)CC6)C5)CC4)CC3)cc21]

The above crude 91b trifluoroacetate (245 mg) was dissolved in 15 mL of THF, and solid sodium bicarbonate (138 mg, 1.64 mmol) was added. The mixture was stirred at room temperature for 20 min, and then tert-butyl 2-oxo-7-azaspiro[3.5]nonane-7-carboxylate (382 mg, 1.60 mmol) and 0.1 mL of acetic acid were added. The mixture was stirred at room temperature for 60 min, and then sodium triacetoxyborohydride (350 mg, 1.65 mmol) was added. The resulting mixture was reacted at room temperature for 16 h. To the reaction system was slowly added 10 mL of saturated sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 108a (160 mg, two-step yield from compound 91a: 66%).

LCMS m/z=663.4 [M+1]⁺

### Step 2: 3-(5-(1-((1-(7-azaspiro[3.5]nonan-2-yl)piperidin-4-yl)methyl)piperidin-4-yl)-3-methyl-2-oxo-2,3-dihydro-1H-benzo[d]imidazol-1-yl)piperidine-2,6-dione (108b) trifluoroacetate

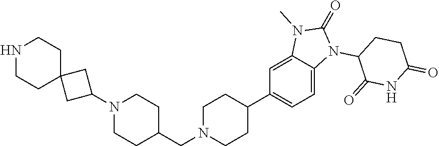 [SMILES: Cn1c(=O)n(C2CCC(=O)NC2=O)c2ccc(C3CCN(CC4CCN(C5CC6(CCNCC6)C5)CC4)CC3)cc21]

108a (80 mg, 0.12 mmol) was dissolved in 10 mL of DCM, and 4 mL of trifluoroacetic acid was added. The mixture was stirred at room temperature for 2 h. The reaction liquid was concentrated under reduced pressure, to afford crude 108b trifluoroacetate (100 mg).

LCMS m/z=563.4 [M+1]⁺

### Step 3: 3-chloro-5-(2-(4-((2-(2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-7-azaspiro[3.5]nonan-7-yl)pyrimidin-4-yl)methoxy)phenyl)propan-2-yl)benzonitrile (Compound 108) trifluoroacetate

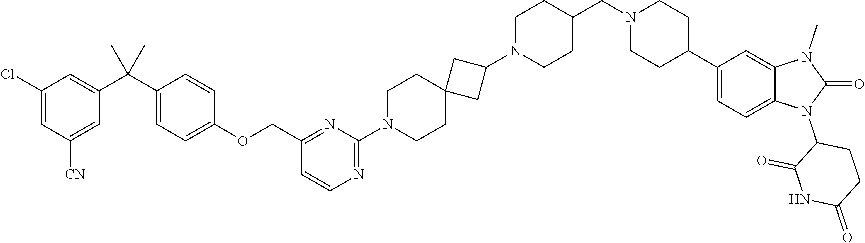 [SMILES: Cn1c(=O)n(C2CCC(=O)NC2=O)c2ccc(C3CCN(CC4CCN(C5CC6(CCN(c7nccc(COc8ccc(C(C)(C)c9cc(Cl)cc(C#N)c9)cc8)n7)CC6)C5)CC4)CC3)cc21]

3-chloro-5-(2-(4-((2-chloropyrimidin-4-yl)methoxy)phenyl)propan-2-yl)benzonitrile (9d) (72 mg, 0.17 mmol) was dissolved in 6 mL of DMF, and the above crude 108b trifluoroacetate (100 mg) and solid potassium bicarbonate (96 mg, 0.96 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 108 trifluoroacetate (35 mg).

¹H NMR (400 MHz, DMSO-d₆) δ 11.08 (s, 1H), 8.34 (d, 1H), 7.88-7.81 (m, 1H), 7.70-7.61 (m, 1H), 7.56-7.47 (m, 1H), 7.23-6.85 (m, 7H), 6.67 (d, 1H), 5.36 (dd, 1H), 4.97 (s, 2H), 3.85-3.55 (m, 7H), 3.55-3.40 (m, 2H), 3.40-3.29 (m, 3H), 3.25-2.98 (m, 4H), 2.98-2.82 (m, 2H), 2.82-2.56 (m, 4H), 2.30-1.72 (m, 12H), 1.72-1.30 (m, 12H).

LCMS m/z=924.3 [M+1]⁺.

### Example 109

### 3-chloro-5-(2-(4-((2-(2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-7-azaspiro[3.5]nonan-7-yl)pyrimidin-4-yl)methoxy)phenyl)propan-2-yl)-2-ethoxybenzonitrile (Compound 109) trifluoroacetate

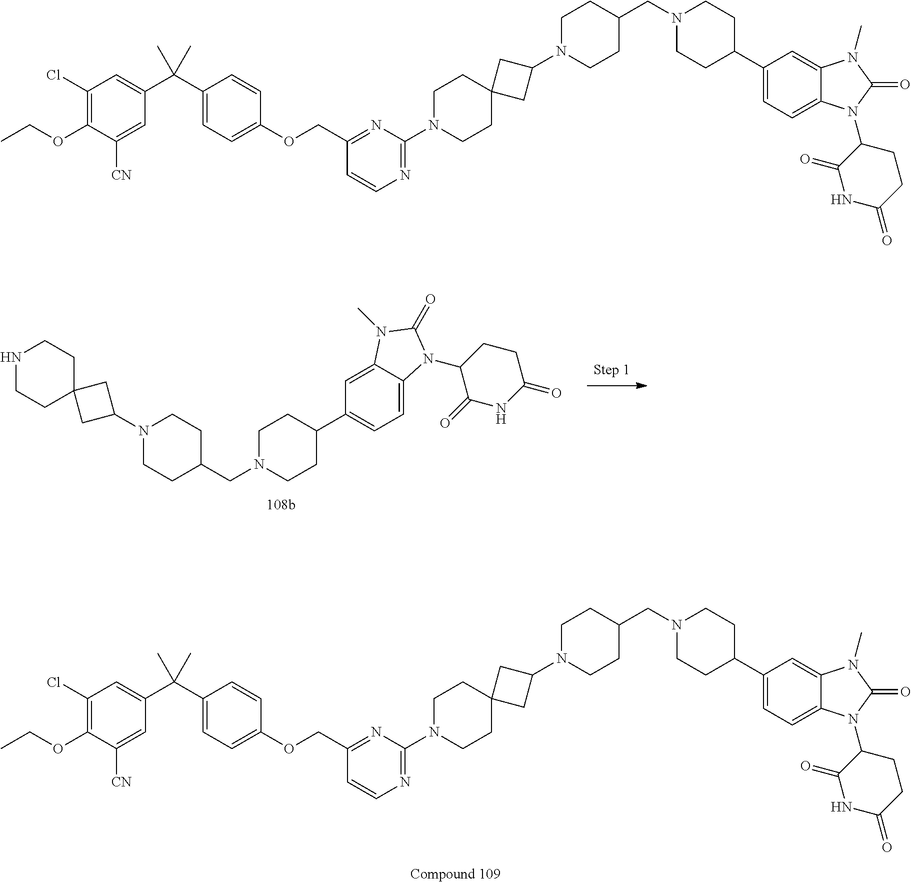 [SMILES: CCOc1c(Cl)cc(C(C)(C)c2ccc(OCc3ccnc(N4CCC5(CC4)CC(N4CCC(CN6CCC(c7ccc8c(c7)n(C)c(=O)n8C7CCC(=O)NC7=O)CC6)CC4)C5)n3)cc2)cc1C#N ; Cn1c(=O)n(C2CCC(=O)NC2=O)c2ccc(C3CCN(CC4CCN(C5CC6(CCNCC6)C5)CC4)CC3)cc21 ; CCOc1c(Cl)cc(C(C)(C)c2ccc(OCc3ccnc(N4CCC5(CC4)CC(N4CCC(CN6CCC(c7ccc8c(c7)n(C)c(=O)n8C7CCC(=O)NC7=O)CC6)CC4)C5)n3)cc2)cc1C#N]

3-chloro-5-(2-(4-((2-chloropyrimidin-4-yl)methoxy)phenyl)propan-2-yl)-2-ethoxybenzonitrile (18g) (80 mg, 0.18 mmol) was dissolved in 6 mL of DMF, and the above crude 108b trifluoroacetate (100 mg) and solid potassium bicarbonate (96 mg, 0.96 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 109 trifluoroacetate (40 mg).

¹H NMR (400 MHz, DMSO-d₆) δ 11.08 (s, 1H), 8.34 (d, 1H), 7.63-7.51 (m, 2H), 7.25-6.85 (m, 7H), 6.67 (d, 1H), 5.43-5.30 (m, 1H), 4.96 (s, 2H), 4.19 (q, 2H), 3.85-3.55 (m, 7H), 3.52-3.40 (m, 2H), 3.40-3.30 (m, 3H), 3.20-2.97 (m, 4H), 2.97-2.80 (m, 2H), 2.80-2.55 (m, 4H), 2.30-1.75 (m, 12H), 1.75-1.20 (m, 15H).

LCMS m/z=968.3 [M+1]⁺

### Example 110

### 5-(1′-((1-(4-((4-(2-(3-chloro-5-cyanophenyl)propan-2-yl)phenoxy)methyl)pyrimidin-2-yl)piperidin-4-yl)methyl)-[4,4′-bipiperidin]-1-yl)-N-(2,6-dioxopiperidin-3-yl)picolinamide (Compound 110) trifluoroacetate

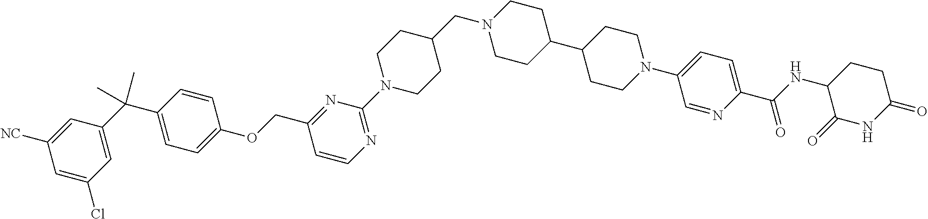 [SMILES: CC(C)(c1ccc(OCc2ccnc(N3CCC(CN4CCC(C5CCN(c6ccc(C(=O)NC7CCC(=O)NC7=O)nc6)CC5)CC4)CC3)n2)cc1)c1cc(Cl)cc(C#N)c1]

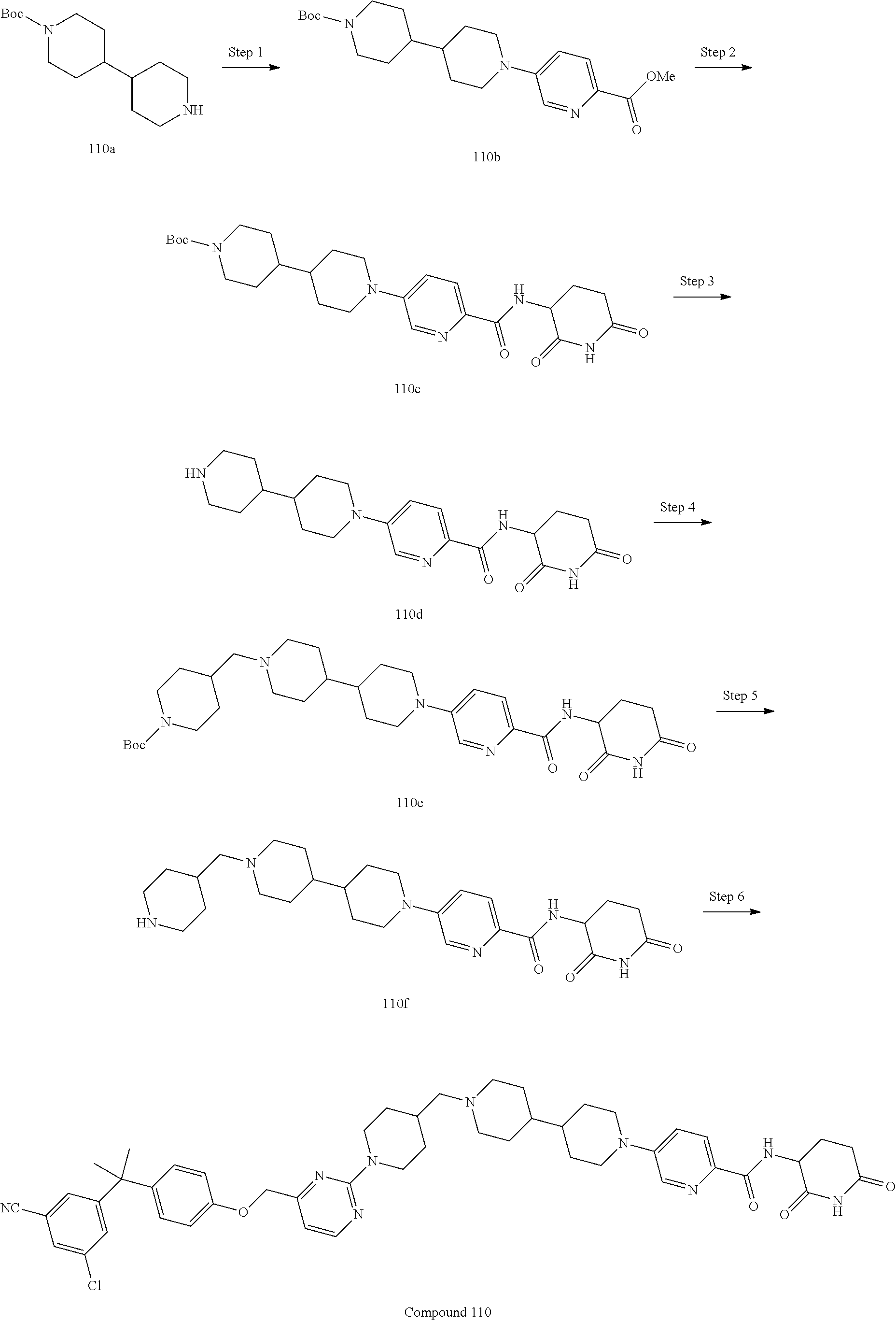

### Step 1: tert-butyl 1′-(6-(methoxycarbonyl)pyridin-3-yl)-[4,4-bipiperidine]-1-carboxylate (110b)

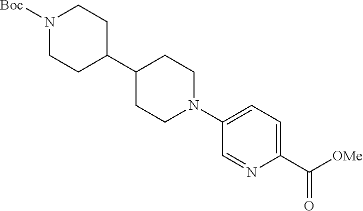 [SMILES: COC(=O)c1ccc(N2CCC(C3CCN(C(=O)OC(C)(C)C)CC3)CC2)cn1]

Tert-butyl [4,4′-bipiperidine]-1-carboxylate (110a) (5.4 g, 20.0 mmol) was added to 60 mL of DMSO, and methyl 5-fluoropyridinecarboxylate (3.46 g, 22.3 mmol) and triethylamine (6.06 g, 60.0 mmol) were successively added. The mixture was warmed to 95° C. and reacted for 3 h. The reaction liquid was cooled to room temperature, and 400 mL of purified water was added to precipitate out a solid. The solid was filtered, and the filter cake was dissolved with 200 mL of dichloromethane, dried over anhydrous sodium sulfate, and concentrated under reduced pressure. The crude product was separated and purified by silica gel column chromatography (dichloromethane/methanol (v/v)=10:1), to afford 110b (6.1 g, yield: 76%).

LCMS m/z=404.3 [M+1]⁺

### Step 2: tert-butyl 1′-(6-((2,6-dioxopiperidin-3-yl)carbamoyl)pyridin-3-yl)-[4,4′-bipiperidine]-1-carboxylate (110c)

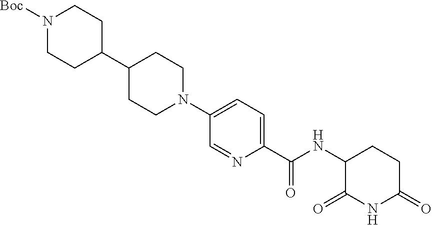 [SMILES: CC(C)(C)OC(=O)N1CCC(C2CCN(c3ccc(C(=O)NC4CCC(=O)NC4=O)nc3)CC2)CC1]

110b (2.0 g, 4.96 mmol) was added to 30 mL of tetrahydrofuran and 8 mL of water, and lithium hydroxide monohydrate (315 mg, 7.50 mmol) was added. The mixture was reacted at room temperature for 16 h. The reaction system was adjusted to pH 6 with 1 mol/L hydrochloric acid, and concentrated under reduced pressure, to afford crude tert-butyl 1′-(6-((2,6-dioxopiperidin-3-yl)carbamoyl)pyridin-3-yl)-[4,4′-bipiperidine]-1-carboxylate (110c) (2.3 g). The above crude (2.3 g) was added to 30 mL of DMF, and 3-aminopiperidine-2,6-dionehydrochloride (1.56 g, 9.48 mmol), DIPEA (2.6 g, 20.1 mmol) and HATU (2.83 g, 7.44 mmol) were successively added. The mixture was reacted at room temperature for 18 h. To the reaction liquid was added 100 mL of purified water to precipitate out a solid. The solid was filtered by suction, and the filter cake was dissolved with 200 mL of dichloromethane, and dried over anhydrous sodium sulfate. The crude product was separated and purified by silica gel column chromatography (dichloromethane/methanol (v/v)=10:1), to afford 110c (1.0 g, yield: 40%).

LCMS m/z=500.3 [M+1]⁺

### Step 3: 5-([4,4′-bipiperidin]-1-yl)-N-(2,6-dioxopiperidin-3-yl)picolinamide (110d) trifluoroacetate

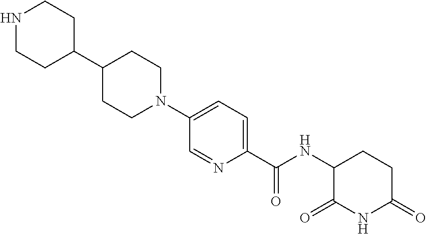 [SMILES: O=C1CCC(NC(=O)c2ccc(N3CCC(C4CCNCC4)CC3)cn2)C(=O)N1]

110c (0.5 g, 1.00 mmol) was dissolved in 10 mL of DCM, and 4 mL of trifluoroacetic acid was added. The mixture was stirred at room temperature for 2 h. The reaction liquid was concentrated under reduced pressure, to afford crude 110d trifluoroacetate (560 mg).

LCMS m/z=400.3 [M+1]⁺

### Step 4: tert-butyl 4-((1′-(6-((2,6-dioxopiperidin-3-yl)carbamoyl)pyridin-3-yl)-[4,4′-bipiperidin]-1-yl)methyl)piperidine-1-carboxylate (110e)

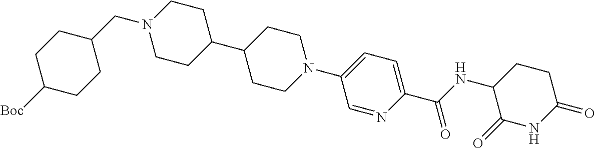 [SMILES: CC(C)(C)OC(=O)C1CCC(CN2CCC(C3CCN(c4ccc(C(=O)NC5CCC(=O)NC5=O)nc4)CC3)CC2)CC1]

The above crude 110d trifluoroacetate (560 mg) was dissolved in 25 mL of THF, and solid sodium bicarbonate (340 mg, 4.05 mmol) was added. The mixture was stirred at room temperature for 20 min, and then tert-butyl 4-formylpiperidine-1-carboxylate (434 mg, 2.03 mmol) and 0.2 mL of acetic acid were added. The mixture was stirred at room temperature for 60 min, and then sodium triacetoxyborohydride (423 mg, 2.00 mmol) was added. The resulting mixture was reacted at room temperature for 16 h. To the reaction system was slowly added 40 mL of saturated aqueous sodium bicarbonate solution, and the mixture was extracted with 150 mL of DCM. The organic phase was washed with 80 mL of water, dried over anhydrous sodium sulfate, and concentrated under reduced pressure. The crude product was separated and purified by silica gel column chromatography (dichloromethane/methanol (v/v)=15:1), to afford 110e (530 mg, two-step yield from compound 110c: 89%).

LCMS m/z=597.4 [M+1]⁺

### Step 5: N-(2,6-dioxopiperidin-3-yl)-5-(1′-(piperidin-4-ylmethyl)-[4,4′-bipiperidin]-1-yl)picolinamide (110f) trifluoroacetate

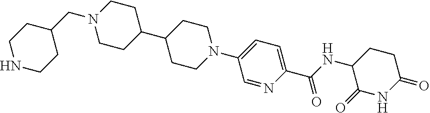 [SMILES: O=C1CCC(NC(=O)c2ccc(N3CCC(C4CCN(CC5CCNCC5)CC4)CC3)cn2)C(=O)N1]

110e (240 mg, 0.40 mmol) was dissolved in 10 mL of DCM, and 4 mL of trifluoroacetic acid was added. The mixture was stirred at room temperature for 2 h. The reaction liquid was concentrated under reduced pressure, to afford crude 110f trifluoroacetate (280 mg).

LCMS m/z=497.3 [M+1]⁺

### Step 6: 5-(1′-((1-(4-((4-(2-(3-chloro-5-cyanophenyl)propan-2-yl)phenoxy)methyl)pyrimidin-2-yl)piperidin-4-yl)methyl)-[4,4′-bipiperidin]-1-yl)-N-(2,6-dioxopiperidin-3-yl)picolinamide (compound 110) trifluoroacetate

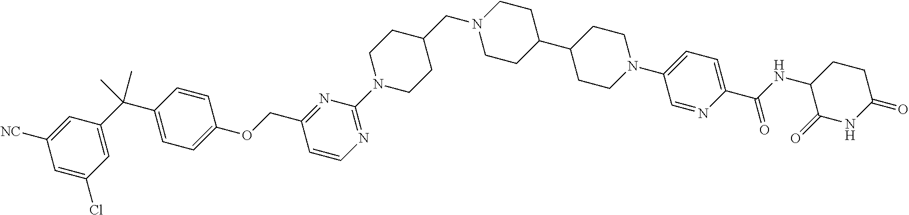 [SMILES: CC(C)(c1ccc(OCc2ccnc(N3CCC(CN4CCC(C5CCN(c6ccc(C(=O)NC7CCC(=O)NC7=O)nc6)CC5)CC4)CC3)n2)cc1)c1cc(Cl)cc(C#N)c1]

3-chloro-5-(2-(4-((2-chloropyrimidin-4-yl)methoxy)phenyl)propan-2-yl)benzonitrile (9d) (96 mg, 0.23 mmol) was dissolved in 6 mL of DMF, and the above crude 110f trifluoroacetate (140 mg) and solid potassium bicarbonate (160 mg, 1.60 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 110 trifluoroacetate (60 mg).

¹H NMR (400 MHz, DMSO-d₆) δ 10.84 (s, 1H), 8.70 (d, 1H), 8.42-8.24 (m, 2H), 7.93-7.80 (m, 2H), 7.70-7.60 (m, 1H), 7.57-7.50 (m, 1H), 7.43 (dd, 1H), 7.25-7.10 (m, 2H), 7.00-6.87 (m, 2H), 6.69 (d, 1H), 4.98 (s, 2H), 4.82-4.55 (m, 3H), 4.07-3.92 (m, 2H), 3.65-3.45 (m, 2H), 3.07-2.70 (m, 9H), 2.60-2.51 (m, 1H), 2.27-1.96 (m, 3H), 1.95-1.72 (m, 6H), 1.64 (s, 6H), 1.59-1.07 (m, 8H).

LCMS m/z=858.3 [M+1]⁺.

### Example 111

### 5-(1′-((1-(4-((4-(2-(3-chloro-5-cyano-4-ethoxyphenyl)propan-2-yl)phenoxy)methyl)pyrimidin-2-yl)piperidin-4-yl)methyl)-[4,4′-bipiperidin]-1-yl)-N-(2,6-dioxopiperidin-3-yl)picolinamide (compound 111) trifluoroacetate

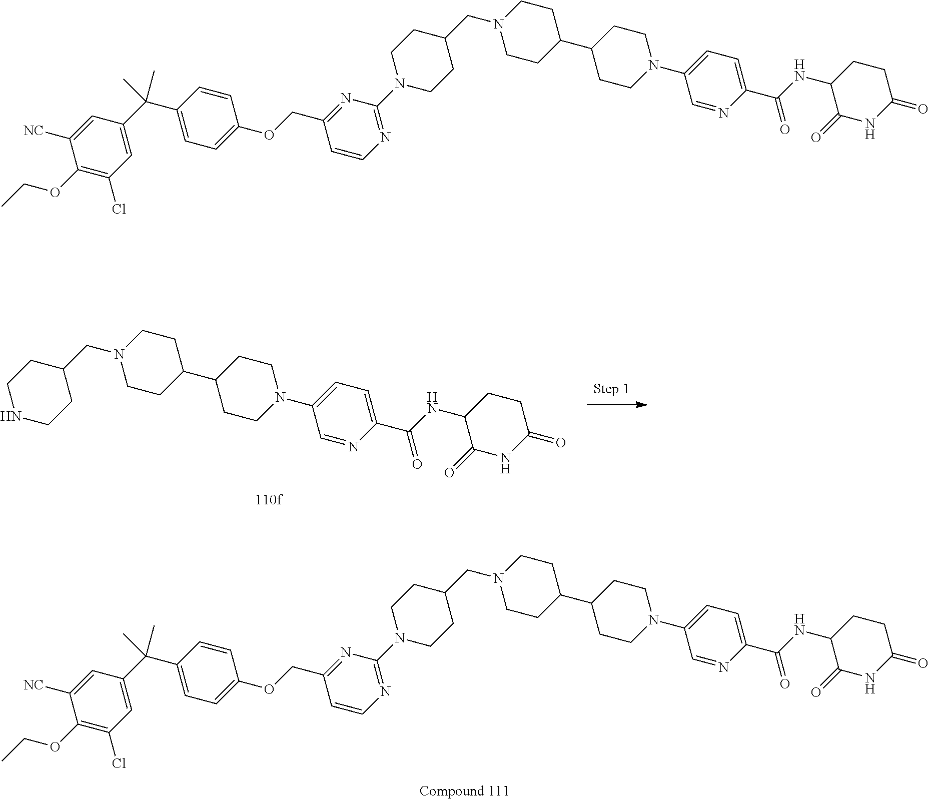 [SMILES: CCOc1c(Cl)cc(C(C)(C)c2ccc(OCc3ccnc(N4CCC(CN5CCC(C6CCN(c7ccc(C(=O)NC8CCC(=O)NC8=O)nc7)CC6)CC5)CC4)n3)cc2)cc1C#N ; O=C1CCC(NC(=O)c2ccc(N3CCC(C4CCN(CC5CCNCC5)CC4)CC3)cn2)C(=O)N1 ; CCOc1c(Cl)cc(C(C)(C)c2ccc(OCc3ccnc(N4CCC(CN5CCC(C6CCN(c7ccc(C(=O)NC8CCC(=O)NC8=O)nc7)CC6)CC5)CC4)n3)cc2)cc1C#N]

3-chloro-5-(2-(4-((2-chloropyrimidin-4-yl)methoxy)phenyl)propan-2-yl)-2-ethoxybenzonitrile (18g) (106 mg, 0.24 mmol) was dissolved in 6 mL of DMF, and the above crude 110f trifluoroacetate (140 mg) and solid potassium bicarbonate (160 mg, 1.60 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 111 trifluoroacetate (70 mg).

¹H NMR (400 MHz, DMSO-d₆) δ 10.84 (s, 1H), 8.70 (d, 1H), 8.40-8.23 (m, 2H), 7.87 (d, 1H), 7.62-7.51 (m, 2H), 7.42 (dd, 1H), 7.22-7.10 (m, 2H), 6.99-6.90 (m, 2H), 6.69 (d, 1H), 4.97 (s, 2H), 4.82-4.57 (m, 3H), 4.19 (q, 2H), 4.05-3.94 (m, 2H), 3.63-3.48 (m, 2H), 3.05-2.70 (m, 9H), 2.62-2.52 (m, 1H), 2.26-1.97 (m, 3H), 1.95-1.72 (m, 6H), 1.63 (s, 6H), 1.58-1.05 (m, 11H).

LCMS m/z=902.4 [M+1]⁺

### Example 112

### 5-(1′-((1-(4-((4-(2-(3-chloro-5-cyano-4-methoxyphenyl)propan-2-yl)phenoxy)methyl)pyrimidin-2-yl)piperidin-4-yl)methyl)-[4,4′-bipiperidin]-1-yl)-N-(2,6-dioxopiperidin-3-yl)picolinamide (compound 112) trifluoroacetate

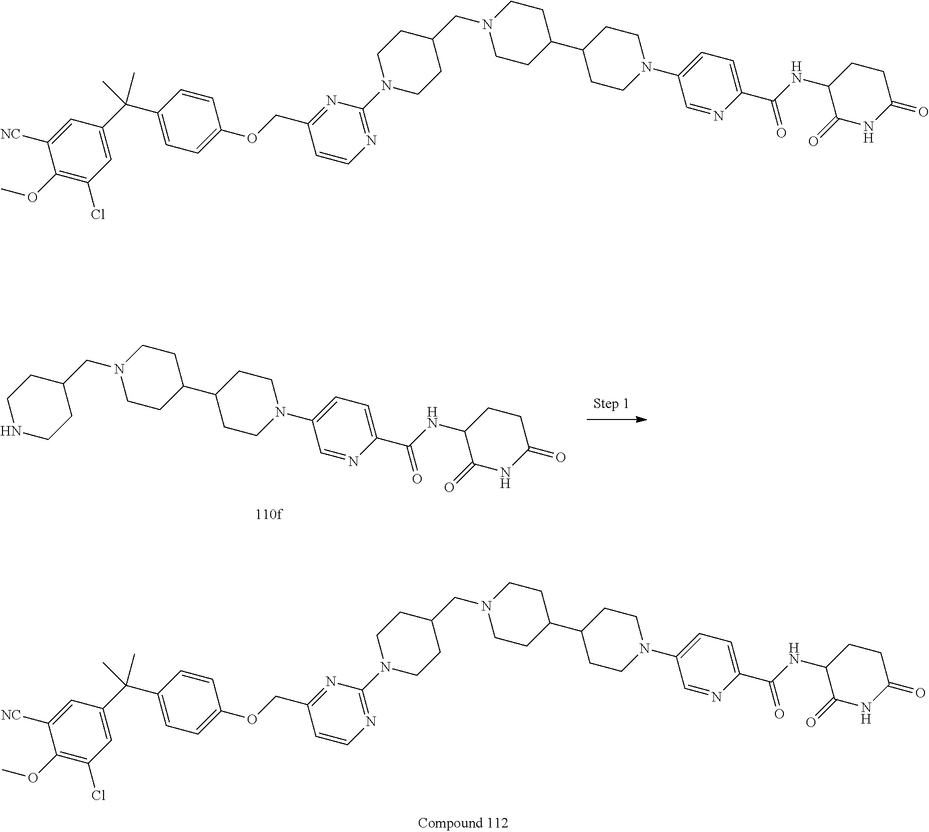 [SMILES: COc1c(Cl)cc(C(C)(C)c2ccc(OCc3ccnc(N4CCC(CN5CCC(C6CCN(c7ccc(C(=O)NC8CCC(=O)NC8=O)nc7)CC6)CC5)CC4)n3)cc2)cc1C#N ; O=C1CCC(NC(=O)c2ccc(N3CCC(C4CCN(CC5CCNCC5)CC4)CC3)cn2)C(=O)N1 ; COc1c(Cl)cc(C(C)(C)c2ccc(OCc3ccnc(N4CCC(CN5CCC(C6CCN(c7ccc(C(=O)NC8CCC(=O)NC8=O)nc7)CC6)CC5)CC4)n3)cc2)cc1C#N]

3-chloro-5-(2-(4-((2-chloropyrimidin-4-yl)methoxy)phenyl)propan-2-yl)-2-methoxybenzonitrile (92e) (106 mg, 0.25 mmol) was dissolved in 6 mL of DMF, and 110f trifluoroacetate (180 mg) and solid potassium bicarbonate (200 mg, 2.00 mmol) were successively added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 112 trifluoroacetate (80 mg).

¹H NMR (400 MHz, DMSO-d₆) δ 10.85 (s, 1H), 8.72 (d, 1H), 8.42-8.26 (m, 2H), 7.87 (d, 1H), 7.65-7.52 (m, 2H), 7.43 (dd, 1H), 7.25-7.10 (m, 2H), 7.01-6.89 (m, 2H), 6.69 (d, 1H), 4.98 (s, 2H), 4.83-4.59 (m, 3H), 4.40-3.96 (m, 5H), 3.64-3.46 (m, 2H), 3.05-2.70 (m, 9H), 2.63-2.50 (m, 1H), 2.26-1.96 (m, 3H), 1.95-1.71 (m, 6H), 1.62 (s, 6H), 1.59-1.05 (m, 8H).

LCMS m/z=888.4 [M+1]⁺

### Example 113

### 3-chloro-5-(2-(4-((2-(2-((4-(1-(2,6-dioxopiperidin-3-yl)-3-methyl-2-oxo-2,3-dihydro-1H-benzo[d]imidazol-5-yl)piperidin-1-yl)methyl)-7-azaspiro[3.5]nonan-7-yl)pyrimidin-4-yl)methoxy)phenyl)propan-2-yl)-2-methoxybenzonitrile (Compound 113) trifluoroacetate

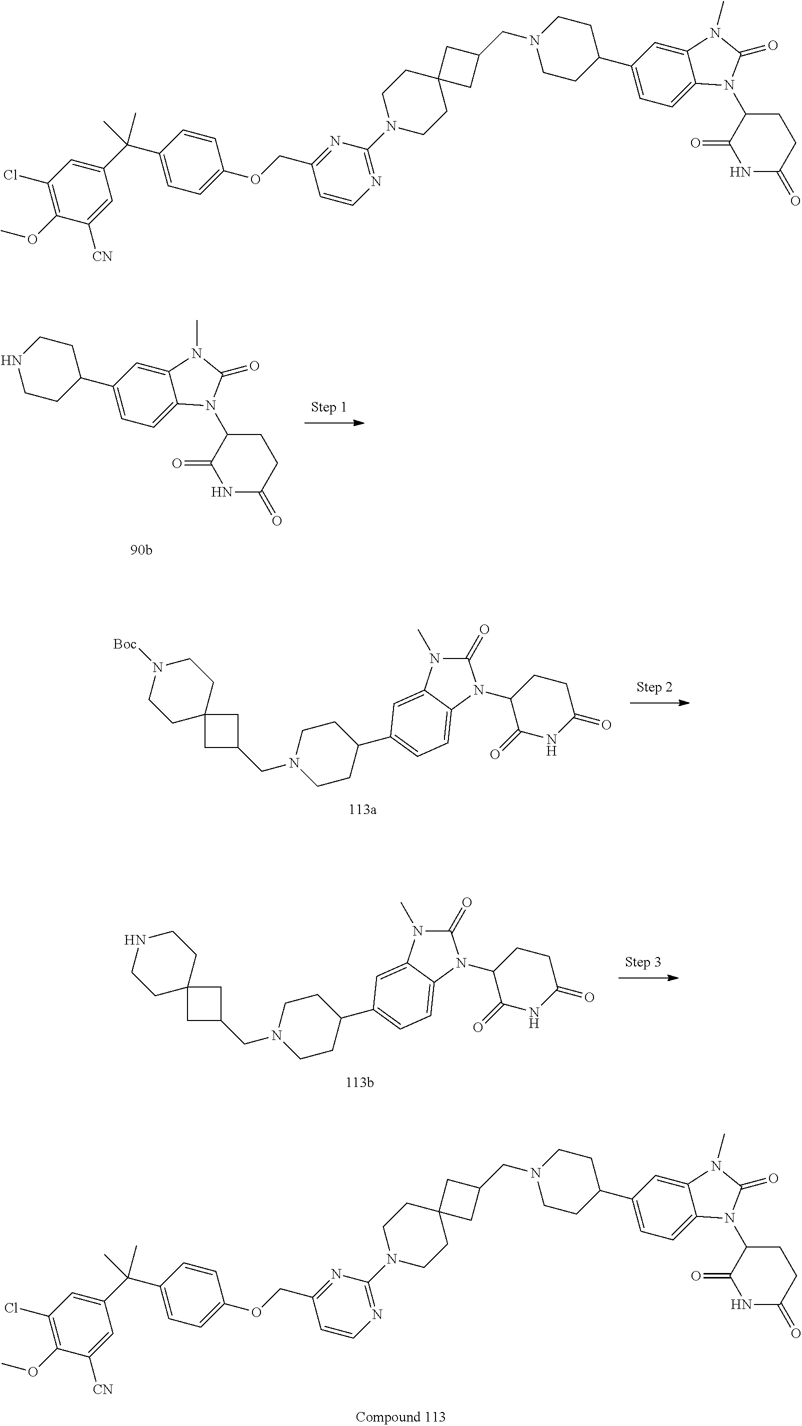 [SMILES: COc1c(Cl)cc(C(C)(C)c2ccc(OCc3ccnc(N4CCC5(CC4)CC(CN4CCC(c6ccc7c(c6)n(C)c(=O)n7C6CCC(=O)NC6=O)CC4)C5)n3)cc2)cc1C#N ; Cn1c(=O)n(C2CCC(=O)NC2=O)c2ccc(C3CCNCC3)cc21 ; Cn1c(=O)n(C2CCC(=O)NC2=O)c2ccc(C3CCN(CC4CC5(CCN(C(=O)OC(C)(C)C)CC5)C4)CC3)cc21 ; Cn1c(=O)n(C2CCC(=O)NC2=O)c2ccc(C3CCN(CC4CC5(CCNCC5)C4)CC3)cc21 ; COc1c(Cl)cc(C(C)(C)c2ccc(OCc3ccnc(N4CCC5(CC4)CC(CN4CCC(c6ccc7c(c6)n(C)c(=O)n7C6CCC(=O)NC6=O)CC4)C5)n3)cc2)cc1C#N]

### Step 1: tert-butyl 2-((4-(1-(2,6-dioxopiperidin-3-yl)-3-methyl-2-oxo-2,3-dihydro-1H-benzo[d]imidazol-5-yl)piperidin-1-yl)methyl)-7-azaspiro[3.5]nonane-7-carboxylate (113a)

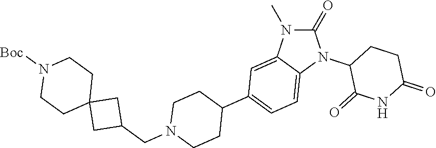 [SMILES: Cn1c(=O)n(C2CCC(=O)NC2=O)c2ccc(C3CCN(CC4CC5(CCN(C(=O)OC(C)(C)C)CC5)C4)CC3)cc21]

The above crude 3-(3-methyl-2-oxo-5-(piperidin-4-yl)-2,3-dihydro-1H-benzo[d]imidazol-1-yl)piperidine-2,6-dione (90b) trifluoroacetate (180 mg) was dissolved in 15 mL of THF, and solid sodium bicarbonate (57 mg, 0.68 mmol) was added. The mixture was stirred at room temperature for 20 min, and then tert-butyl 2-formyl-7-azaspiro[3.5]nonane-7-carboxylate (see 2021023105 for the synthetic method) (171 mg, 0.67 mmol) and 0.1 mL of acetic acid were successively added. The mixture was stirred at room temperature for 60 min, and then sodium triacetoxyborohydride (144 mg, 0.68 mmol) was added. The resulting mixture was reacted at room temperature for 16 h. To the reaction system was slowly added 10 mL of saturated aqueous sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 113a (130 mg, two-step yield from compound 90a: 66%).

LCMS m/z=580.4 [M+1]⁺

### Step 2: 3-(5-(1-((7-azaspiro[3.5]nonan-2-yl)methyl)piperidin-4-yl)-3-methyl-2-oxo-2,3-dihydro-1H-benzo[d]imidazol-1-yl)piperidine-2,6-dione (113b) trifluoroacetate

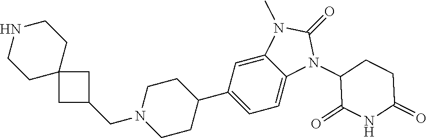 [SMILES: Cn1c(=O)n(C2CCC(=O)NC2=O)c2ccc(C3CCN(CC4CC5(CCNCC5)C4)CC3)cc21]

113a (120 mg, 0.21 mmol) was dissolved in 10 mL of DCM, and 4 mL of trifluoroacetic acid was added. The mixture was stirred at room temperature for 2 h. The reaction liquid was concentrated under reduced pressure, to afford crude 113b trifluoroacetate (160 mg).

LCMS m/z=480.3 [M+1]⁺

### Step 3: 3-chloro-5-(2-(4-((2-(2-((4-(1-(2,6-dioxopiperidin-3-yl)-3-methyl-2-oxo-2,3-dihydro-1H-benzo[d]imidazol-5-yl)piperidin-1-yl)methyl)-7-azaspiro[3.5]nonan-7-yl)pyrimidin-4-yl)methoxy)phenyl)propan-2-yl)-2-methoxybenzonitrile (Compound 113) trifluoroacetate

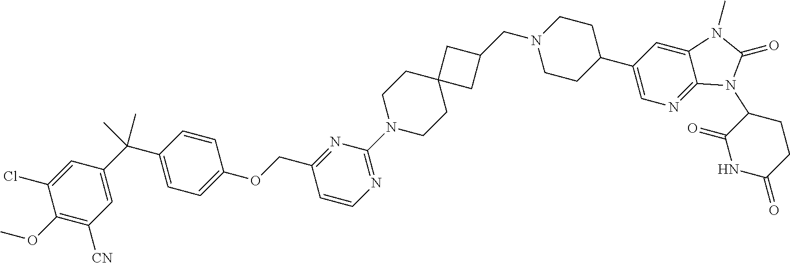 [SMILES: COc1c(Cl)cc(C(C)(C)c2ccc(OCc3ccnc(N4CCC5(CC4)CC(CN4CCC(c6cnc7c(c6)n(C)c(=O)n7C6CCC(=O)NC6=O)CC4)C5)n3)cc2)cc1C#N]

3-chloro-5-(2-(4-((2-chloropyrimidin-4-yl)methoxy)phenyl)propan-2-yl)-2-methoxybenzonitrile (92e) (121 mg, 0.28 mmol) was dissolved in 6 mL of DMF, and the above crude 113b trifluoroacetate (160 mg) and solid potassium bicarbonate (155 mg, 1.55 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 113 trifluoroacetate (30 mg).

¹H NMR (400 MHz, DMSO-d₆) δ 11.09 (s, 1H), 8.34 (d, 1H), 7.66-7.50 (m, 2H), 7.25-6.85 (m, 7H), 6.66 (d, 1H), 5.36 (dd, 1H), 4.97 (s, 2H), 3.96 (s, 3H), 3.83-3.70 (m, 2H), 3.70-3.61 (m, 2H), 3.57-3.45 (m, 2H), 3.39-3.32 (m, 3H), 3.32-3.17 (m, 2H), 3.15-2.57 (m, 7H), 2.20-1.80 (m, 7H), 1.75-1.40 (m, 12H).

LCMS m/z=871.4 [M+1]⁺.

### Example 114

### 3-chloro-5-(2-(4-((2-(4-(((1-(1-(2,6-dioxopiperidin-3-yl)-3-methyl-2-oxo-2,3-dihydro-1H-benzo[d]imidazol-5-yl)piperidin-4-yl)(methyl)amino)methyl)piperidin-1-yl)pyrimidin-4-yl)methoxy)phenyl)propan-2-yl)benzonitrile (Compound 114) trifluoroacetate

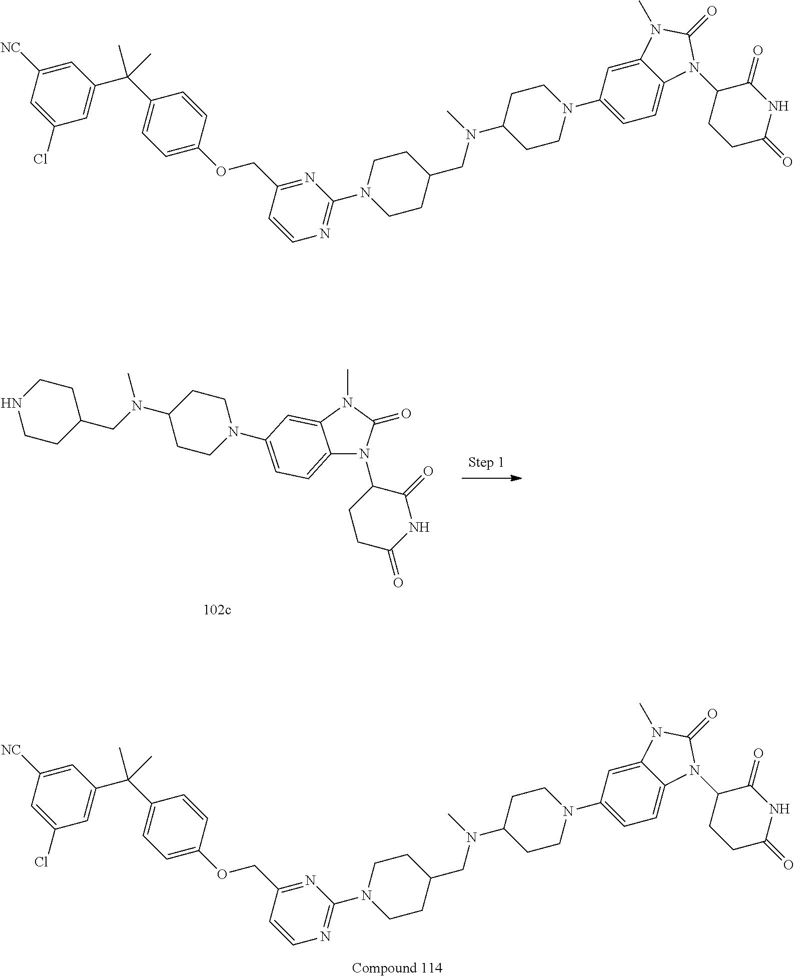 [SMILES: CN(CC1CCN(c2nccc(COc3ccc(C(C)(C)c4cc(Cl)cc(C#N)c4)cc3)n2)CC1)C1CCN(c2ccc3c(c2)n(C)c(=O)n3C2CCC(=O)NC2=O)CC1 ; CN(CC1CCNCC1)C1CCN(c2ccc3c(c2)n(C)c(=O)n3C2CCC(=O)NC2=O)CC1 ; CN(CC1CCN(c2nccc(COc3ccc(C(C)(C)c4cc(Cl)cc(C#N)c4)cc3)n2)CC1)C1CCN(c2ccc3c(c2)n(C)c(=O)n3C2CCC(=O)NC2=O)CC1]

The above crude 102c trifluoroacetate (2.50 g) was dissolved in 20 mL of DMF, and solid potassium bicarbonate (3.0 g, 30 mmol) was added. The mixture was stirred at room temperature for 0.5 h, and then 3-chloro-5-(2-(4-((2-chloropyrimidin-4-yl)methoxy)phenyl)propan-2-yl)benzonitrile (9d) (2.00 g, 4.94 mmol) was added. The resulting mixture was warmed to 80° C. and stirred for 4 h. The reaction liquid was cooled to room temperature, and 20 mL of water was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 114 trifluoroacetate (0.6 g).

¹H NMR (400 MHz, DMSO-d₆) δ 11.06 (s, 1H), 8.41-8.31 (m, 1H), 7.88-7.78 (m, 1H), 7.68-7.60 (m, 1H), 7.56-7.48 (m, 1H), 7.24-7.10 (m, 2H), 7.04-6.86 (m, 4H), 6.78-6.62 (m, 2H), 5.31 (dd, 1H), 4.98 (s, 2H), 4.77-4.62 (m, 2H), 3.90-3.75 (m, 2H), 3.51-3.33 (m, 2H), 3.32 (s, 3H), 3.24-3.12 (m, 1H), 3.04-2.75 (m, 8H), 2.75-2.56 (m, 2H), 2.23-1.72 (m, 8H), 1.64 (s, 6H), 1.30-1.09 (m, 2H).

LCMS m/z=830.3 [M+1]⁺.

### Example 115

### 3-chloro-5-(2-(4-((2-(2-(4-((4-(1-(2,6-dioxopiperidin-3-yl)-3-methyl-2-oxo-2,3-dihydro-1H-benzo[d]imidazol-4-yl)piperidin-1-yl)methyl)-4-fluoropiperidin-1-yl)-7-azaspiro[3.5]nonan-7-yl)pyrimidin-4-yl)methoxy)phenyl)propan-2-yl)benzonitrile (Compound 115) trifluoroacetate

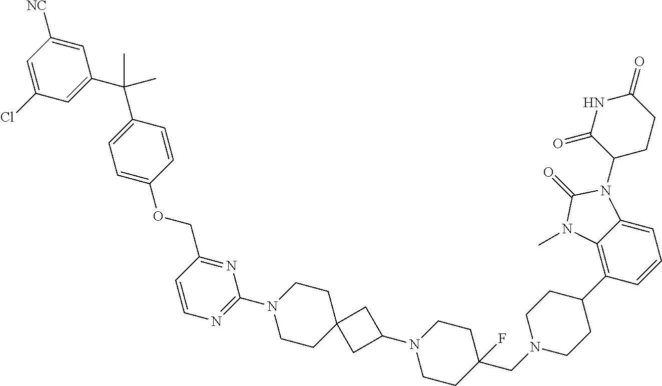 [SMILES: Cn1c(=O)n(C2CCC(=O)NC2=O)c2cccc(C3CCN(CC4(F)CCN(C5CC6(CCN(c7nccc(COc8ccc(C(C)(C)c9cc(Cl)cc(C#N)c9)cc8)n7)CC6)C5)CC4)CC3)c21]

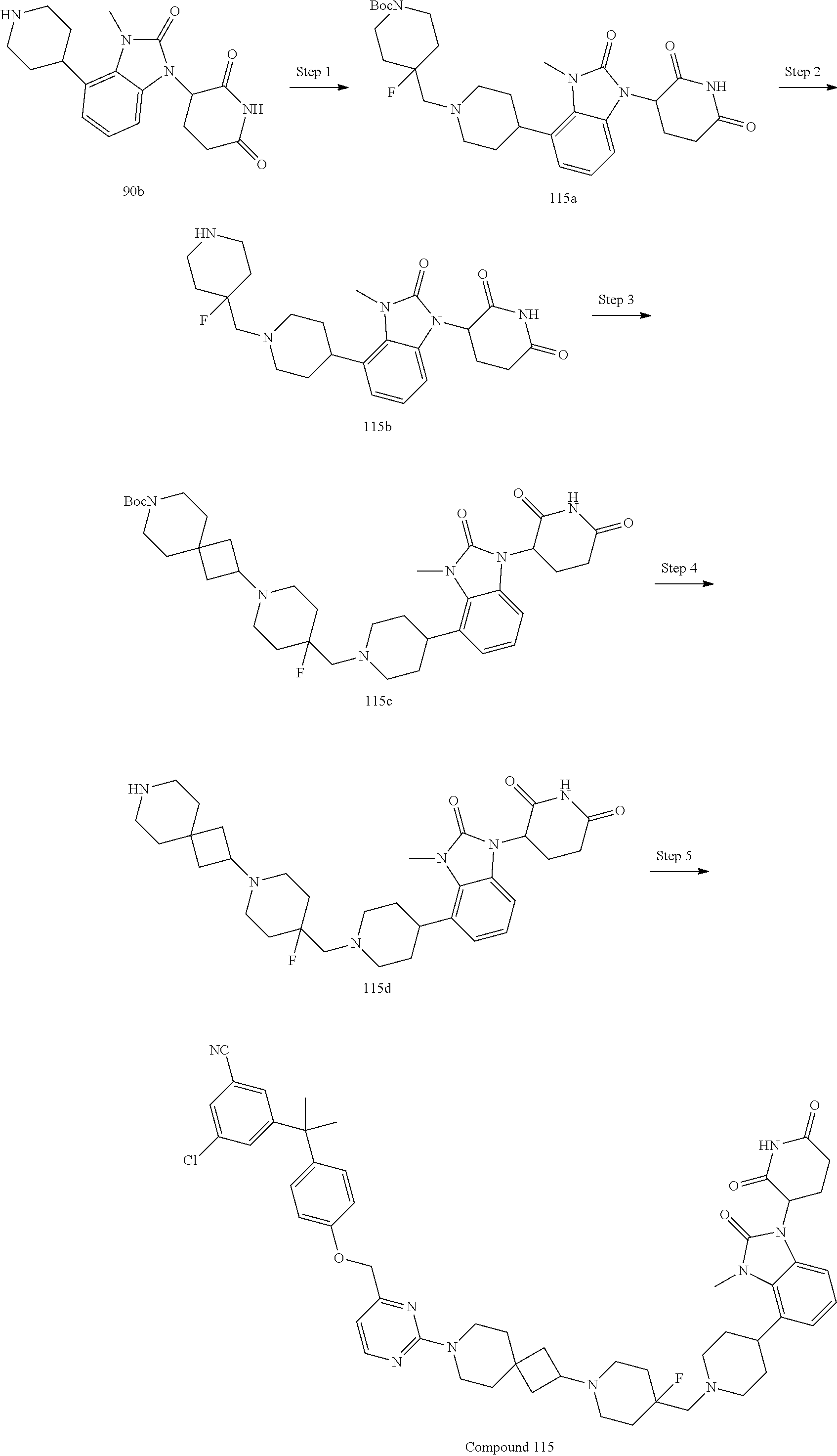 [SMILES: Cn1c(=O)n(C2CCC(=O)NC2=O)c2cccc(C3CCNCC3)c21 ; Cn1c(=O)n(C2CCC(=O)NC2=O)c2cccc(C3CCN(CC4(F)CCN(C(=O)OC(C)(C)C)CC4)CC3)c21 ; Cn1c(=O)n(C2CCC(=O)NC2=O)c2cccc(C3CCN(CC4(F)CCNCC4)CC3)c21 ; Cn1c(=O)n(C2CCC(=O)NC2=O)c2cccc(C3CCN(CC4(F)CCN(C5CC6(CCN(C(=O)OC(C)(C)C)CC6)C5)CC4)CC3)c21 ; Cn1c(=O)n(C2CCC(=O)NC2=O)c2cccc(C3CCN(CC4(F)CCN(C5CC6(CCNCC6)C5)CC4)CC3)c21 ; Cn1c(=O)n(C2CCC(=O)NC2=O)c2cccc(C3CCN(CC4(F)CCN(C5CC6(CCN(c7nccc(COc8ccc(C(C)(C)c9cc(Cl)cc(C#N)c9)cc8)n7)CC6)C5)CC4)CC3)c21]

### Step 1: tert-butyl 4-((4-(1-(2,6-dioxopiperidin-3-yl)-3-methyl-2-oxo-2,3-dihydro-1H-benzo[d]imidazol-4-yl)piperidin-1-yl)methyl)-4-fluoropiperidine-1-carboxylate (115a)

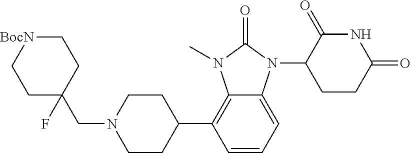 [SMILES: Cn1c(=O)n(C2CCC(=O)NC2=O)c2cccc(C3CCN(CC4(F)CCN(C(=O)OC(C)(C)C)CC4)CC3)c21]

The above crude 90b trifluoroacetate (900 mg) was dissolved in a mixed solvent of 10 mL of tetrahydrofuran and 10 mL of 1,2-dichloroethane, and to the reaction system were added dimethyl sulfoxide (0.5 mL) and solid sodium bicarbonate (0.84 g, 10.0 mmol). The mixture was stirred at room temperature for 0.5 h, and then tert-butyl 4-fluoro-4-formylpiperidine-1-carboxylate (see WO 2014063587 for the synthetic method) (1.39 g, 6.01 mmol) and glacial acetic acid (1.0 mL) were added. The mixture was stirred at room temperature for 2 h, and then sodium triacetoxyborohydride (1.27 g, 6.0 mmol) was added. The resulting mixture was reacted at room temperature for 16 h. To the reaction liquid was added a saturated sodium bicarbonate solution (20 mL) to quench the reaction, and the mixture was extracted with ethyl acetate (50 mL×3). The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 115a (1.30 g).

### Step 2: 3-(4-(1-((4-fluoropiperidin-4-yl)methyl)piperidin-4-yl)-3-methyl-2-oxo-2,3-dihydro-1H-benzo[d]imidazol-1-yl)piperidine-2,6-dione (115b) trifluoroacetate

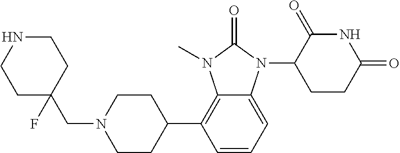 [SMILES: Cn1c(=O)n(C2CCC(=O)NC2=O)c2cccc(C3CCN(CC4(F)CCNCC4)CC3)c21]

115a (1.30 g, 2.33 mmol) was dissolved in 10 mL of dichloromethane, and trifluoroacetic acid (15.30 g, 134.20 mmol) was added. The mixture was stirred at room temperature for 2 h. The reaction system was concentrated under reduced pressure, to afford crude 115b trifluoroacetate (1.1 g).

### Step 3: tert-butyl 2-(4-((4-(1-(2,6-dioxopiperidin-3-yl)-3-methyl-2-oxo-2,3-dihydro-1H-benzo[d]imidazol-4-yl)piperidin-1-yl)methyl)-4-fluoropiperidin-1-yl)-7-azaspiro[3.5]nonane-7-carboxylate (115c)

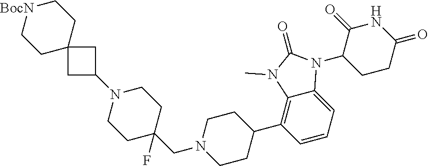 [SMILES: Cn1c(=O)n(C2CCC(=O)NC2=O)c2cccc(C3CCN(CC4(F)CCN(C5CC6(CCN(C(=O)OC(C)(C)C)CC6)C5)CC4)CC3)c21]

The above crude 115b trifluoroacetate (318 mg) was dissolved in 5 mL of tetrahydrofuran, and dimethyl sulfoxide (0.5 mL) and solid sodium bicarbonate (0.151 g, 1.8 mmol) were added. The mixture was stirred at room temperature for 0.5 h, and then tert-butyl 2-oxo-7-azaspiro[3.5]nonane-7-carboxylate (0.48 g, 2.00 mmol) and glacial acetic acid (1.0 mL) were added. The resulting mixture was warmed to 60° C. and reacted for 2 h. The reaction liquid was cooled to room temperature, and sodium triacetoxyborohydride (0.50 g, 2.36 mmol) was added. The resulting mixture was reacted at room temperature for 16 h. The reaction liquid was quenched with a saturated sodium bicarbonate solution (20 mL), and extracted with ethyl acetate (30 mL×3). The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 115c (83 mg, two-step yield from compound 115a: 18%).

### Step 4: 3-(4-(1-((4-fluoro-1-(7-azaspiro[3.5]nonan-2-yl)piperidin-4-yl)methyl)piperidin-4-yl)-3-methyl-2-oxo-2,3-dihydro-1H-benzo[d]imidazol-1-yl)piperidine-2,6-dione (115d) trifluoroacetate

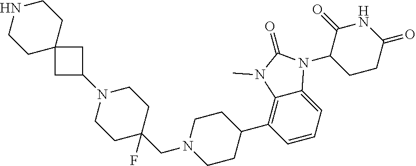 [SMILES: Cn1c(=O)n(C2CCC(=O)NC2=O)c2cccc(C3CCN(CC4(F)CCN(C5CC6(CCNCC6)C5)CC4)CC3)c21]

115c (83 mg, 0.122 mmol) was dissolved in 2 mL of dichloromethane, and trifluoroacetic acid (1.53 g, 13.42 mmol) was added. The mixture was stirred at room temperature for 2 h. The reaction liquid was concentrated under reduced pressure, to afford crude 115d trifluoroacetate (83 mg).

### Step 5: 3-chloro-5-(2-(4-((2-(2-(4-((4-(1-(2,6-dioxopiperidin-3-yl)-3-methyl-2-oxo-2,3-dihydro-1H-benzo[d]imidazol-4-yl)piperidin-1-yl)methyl)-4-fluoropiperidin-1-yl)-7-azaspiro[3.5]nonan-7-yl)pyrimidin-4-yl)methoxy)phenyl)propan-2-yl)benzonitrile (Compound 115) trifluoroacetate

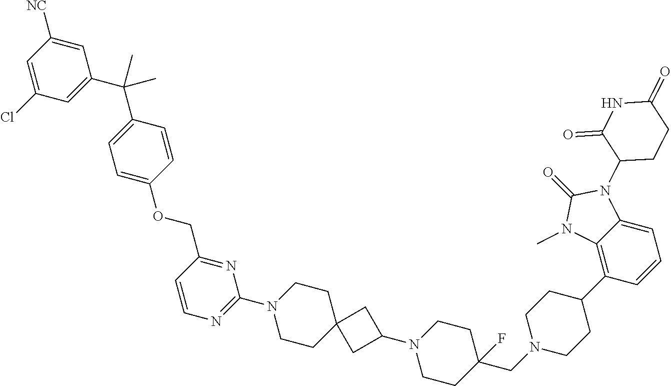 [SMILES: Cn1c(=O)n(C2CCC(=O)NC2=O)c2cccc(C3CCN(CC4(F)CCN(C5CC6(CCN(c7nccc(COc8ccc(C(C)(C)c9cc(Cl)cc(C#N)c9)cc8)n7)CC6)C5)CC4)CC3)c21]

The above crude 115d trifluoroacetate (83 mg) was dissolved in 5 mL of N,N dimethylformamide, and solid potassium bicarbonate (80 mg, 0.80 mmol) was added. The mixture was stirred at room temperature for 0.5 h, and then 3-chloro-5-(2-(4-((2-chloropyrimidin-4-yl)methoxy)phenyl)propan-2-yl)benzonitrile (9d) (95 mg, 0.23 mmol) was added. The resulting mixture was warmed to 80° C. and reacted for 4 h. The reaction liquid was cooled to room temperature, and 10 mL of water was added. The mixture was extracted with ethyl acetate (20 mL×3), and the organic phase was washed with 10 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 115 trifluoroacetate (35 mg).

¹H NMR (400 MHz, DMSO-d₆) δ 11.09 (s, 1H), 8.34 (d, 1H), 7.88-7.78 (m, 1H), 7.70-7.60 (m, 1H), 7.57-7.48 (m, 1H), 7.24-6.85 (m, 7H), 6.68 (d, 1H), 5.39 (dd, 1H), 4.97 (s, 2H), 3.95-3.22 (m, 17H), 3.06-2.81 (m, 3H), 2.80-2.56 (m, 2H), 2.49-2.31 (m, 2H), 2.30-1.90 (m, 11H), 1.76-1.44 (m, 10H).

LCMS m/z=942.4 [M+1]⁺

### Example 116

### 3-chloro-5-(2-(4-((2-(4-((4-(1-(2,6-dioxopiperidin-3-yl)-3-methyl-2-oxo-2,3-dihydro-1H-benzo[d]imidazol-5-yl)-[1,4′-bipiperidin]-1′-yl)methyl)piperidin-1-yl)pyrimidin-4-yl)methoxy)phenyl)propan-2-yl)-2-methoxybenzonitrile (Compound 116) trifluoroacetate

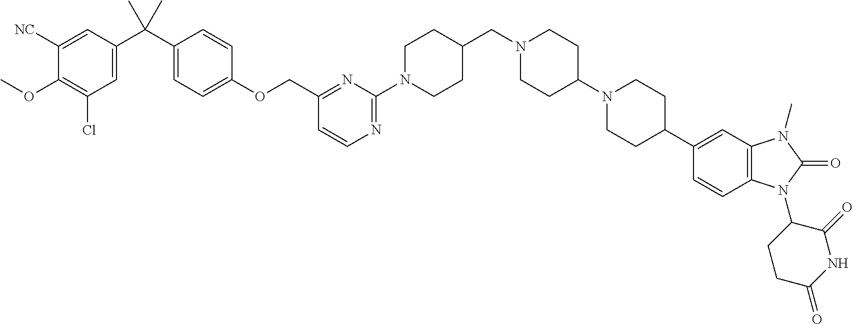 [SMILES: COc1c(Cl)cc(C(C)(C)c2ccc(OCc3ccnc(N4CCC(CN5CCC(N6CCC(c7ccc8c(c7)n(C)c(=O)n8C7CCC(=O)NC7=O)CC6)CC5)CC4)n3)cc2)cc1C#N]

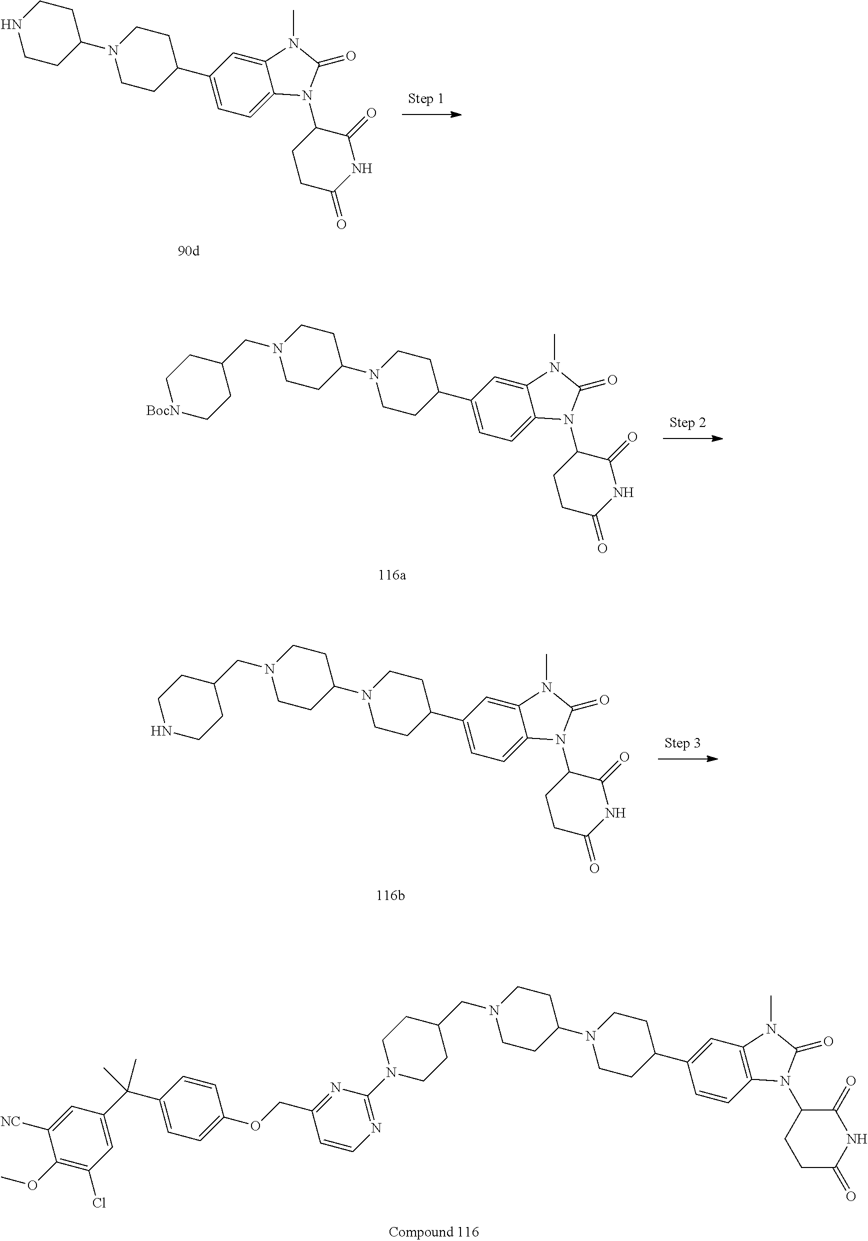 [SMILES: Cn1c(=O)n(C2CCC(=O)NC2=O)c2ccc(C3CCN(C4CCNCC4)CC3)cc21 ; Cn1c(=O)n(C2CCC(=O)NC2=O)c2ccc(C3CCN(C4CCN(CC5CCN(C(=O)OC(C)(C)C)CC5)CC4)CC3)cc21 ; Cn1c(=O)n(C2CCC(=O)NC2=O)c2ccc(C3CCN(C4CCN(CC5CCNCC5)CC4)CC3)cc21 ; COc1c(Cl)cc(C(C)(C)c2ccc(OCc3ccnc(N4CCC(CN5CCC(N6CCC(c7ccc8c(c7)n(C)c(=O)n8C7CCC(=O)NC7=O)CC6)CC5)CC4)n3)cc2)cc1C#N]

### Step 1: tert-butyl 4-((4-(1-(2,6-dioxopiperidin-3-yl)-3-methyl-2-oxo-2,3-dihydro-1H-benzo[d]imidazol-5-yl)-[1,4′-bipiperidin]-1′-yl)methyl)piperidine-1-carboxylate (116a)

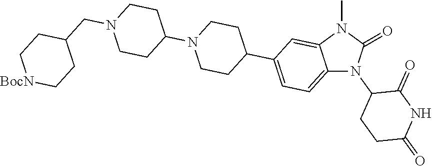 [SMILES: Cn1c(=O)n(C2CCC(=O)NC2=O)c2ccc(C3CCN(C4CCN(CC5CCN(C(=O)OC(C)(C)C)CC5)CC4)CC3)cc21]

The above crude 90d trifluoroacetate (40 mg) was dissolved in 5 mL of tetrahydrofuran, and dimethyl sulfoxide (0.5 mL) and solid sodium bicarbonate (0.011 g, 0.13 mmol) were added. The mixture was stirred at room temperature for 0.5 h, and then tert-butyl 4-formylpiperidine-1-carboxylate (0.053 g, 0.25 mmol) and glacial acetic acid (0.2 mL) were added. The mixture was reacted at room temperature for 2 h, and then sodium triacetoxyborohydride (0.053 g, 0.25 mmol) was added. The resulting mixture was reacted at room temperature for 16 h. The reaction liquid was quenched with a saturated sodium bicarbonate solution (10 mL), and extracted with ethyl acetate (20 mL×3). The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 116a (40 mg, two-step yield from compound 90c: >99%).

### Step 2: 3-(3-methyl-2-oxo-5-(1′-(piperidin-4-ylmethyl)-[1,4′-bipiperidin]-4-yl)-2,3-dihydro-1H-benzo[d]imidazol-1-yl)piperidine-2,6-dione (116b) trifluoroacetate

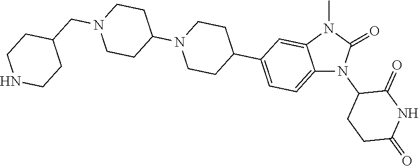 [SMILES: Cn1c(=O)n(C2CCC(=O)NC2=O)c2ccc(C3CCN(C4CCN(CC5CCNCC5)CC4)CC3)cc21]

116a (40 mg, 0.064 mmol) was dissolved in 2 mL of dichloromethane, and trifluoroacetic acid (1.53 g, 13.42 mmol) was added. The mixture was reacted at room temperature for 2 h. The reaction system was concentrated under reduced pressure, to afford crude 116b trifluoroacetate (40 mg).

### Step 3: 3-chloro-5-(2-(4-((2-(4-((4-(1-(2,6-dioxopiperidin-3-yl)-3-methyl-2-oxo-2,3-dihydro-1H-benzo[d]imidazol-5-yl)-[1,4′-bipiperidin]-1′-yl)methyl)piperidin-1-yl)pyrimidin-4-yl)methoxy)phenyl)propan-2-yl)-2-methoxybenzonitrile (Compound 116) trifluoroacetate

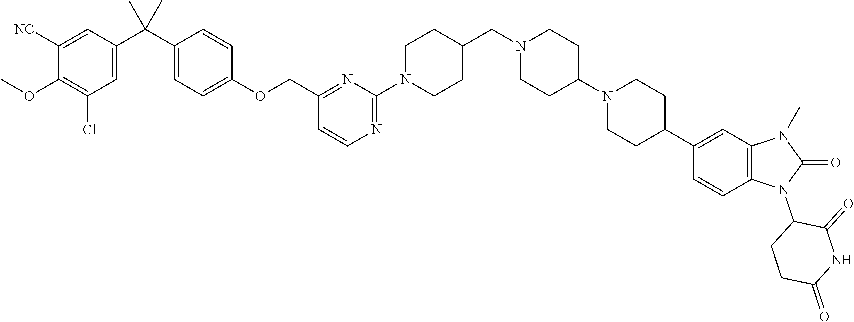 [SMILES: COc1c(Cl)cc(C(C)(C)c2ccc(OCc3ccnc(N4CCC(CN5CCC(N6CCC(c7ccc8c(c7)n(C)c(=O)n8C7CCC(=O)NC7=O)CC6)CC5)CC4)n3)cc2)cc1C#N]

The above crude 116b trifluoroacetate (40 mg) was dissolved in 5 mL of DMF, and solid potassium bicarbonate (63 mg, 0.63 mmol) was added. The mixture was reacted at room temperature for 0.5 h, and then 3-chloro-5-(2-(4-((2-chloropyrimidin-4-yl)methoxy)phenyl)propan-2-yl)-2-methoxybenzonitrile (92e) (50 mg, 0.12 mmol) was added. The resulting mixture was warmed to 80° C. and reacted for 4 h. The reaction liquid was cooled to room temperature, and water (10 mL) was added. The mixture was extracted with ethyl acetate (20 mL×3), and the organic phase was washed with 10 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 116 trifluoroacetate (23 mg).

¹H NMR (400 MHz, DMSO-d₆) δ 11.08 (s, 1H), 8.36 (d, 1H), 7.63-7.53 (m, 2H), 7.24-7.13 (m, 2H), 7.12-6.99 (m, 2H), 6.97-6.88 (m, 3H), 6.69 (d, 1H), 5.40-5.32 (m, 1H), 4.97 (s, 2H), 4.74-4.61 (m, 2H), 3.96 (s, 3H), 3.80-3.65 (m, 2H), 3.65-3.52 (m, 4H), 3.34 (s, 3H), 3.25-3.10 (m, 2H), 3.10-2.83 (m, 7H), 2.75-2.57 (m, 2H), 2.38-2.24 (m, 2H), 2.20-1.89 (m, 8H), 1.87-1.75 (m, 2H), 1.63 (s, 6H), 1.25-1.08 (m, 2H).

LCMS m/z=457.8 [M/2+1]⁺.

### Example 118

### 3-chloro-5-(2-(4-((2-(4-((2-(1-(2,6-dioxopiperidin-3-yl)-3-methyl-2-oxo-2,3-dihydro-1H-benzo[d]imidazol-5-yl)-2,7-diazaspiro[3.5]nonan-7-yl)methyl)piperidin-1-yl)pyrimidin-4-yl)methoxy)phenyl)propan-2-yl)-2-ethoxybenzonitrile (Compound 118) trifluoroacetate

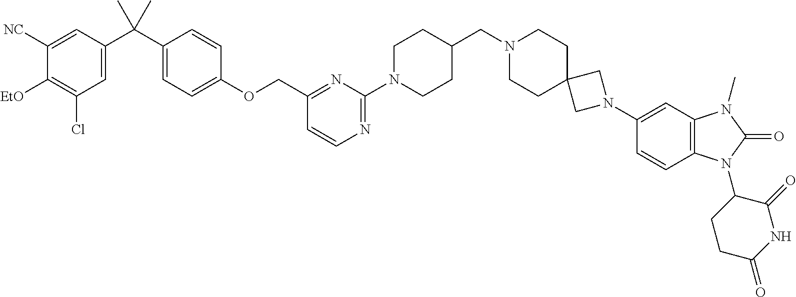 [SMILES: CCOc1c(Cl)cc(C(C)(C)c2ccc(OCc3ccnc(N4CCC(CN5CCC6(CC5)CN(c5ccc7c(c5)n(C)c(=O)n7C5CCC(=O)NC5=O)C6)CC4)n3)cc2)cc1C#N]

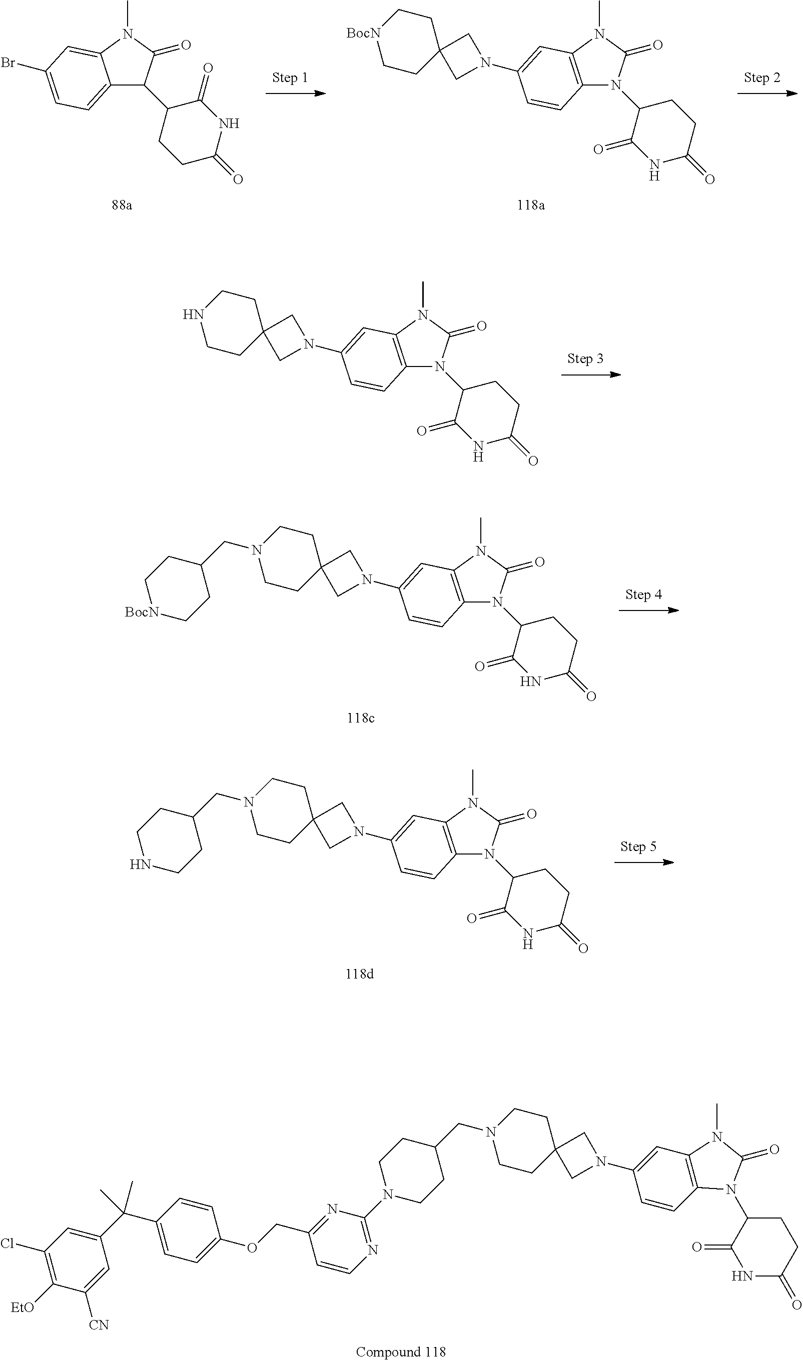 [SMILES: CN1C(=O)C(C2CCC(=O)NC2=O)c2ccc(Br)cc21 ; Cn1c(=O)n(C2CCC(=O)NC2=O)c2ccc(N3CC4(CCN(C(=O)OC(C)(C)C)CC4)C3)cc21 ; Cn1c(=O)n(C2CCC(=O)NC2=O)c2ccc(N3CC4(CCNCC4)C3)cc21 ; Cn1c(=O)n(C2CCC(=O)NC2=O)c2ccc(N3CC4(CCN(CC5CCN(C(=O)OC(C)(C)C)CC5)CC4)C3)cc21 ; Cn1c(=O)n(C2CCC(=O)NC2=O)c2ccc(N3CC4(CCN(CC5CCNCC5)CC4)C3)cc21 ; CCOc1c(Cl)cc(C(C)(C)c2ccc(OCc3ccnc(N4CCC(CN5CCC6(CC5)CN(c5ccc7c(c5)n(C)c(=O)n7C5CCC(=O)NC5=O)C6)CC4)n3)cc2)cc1C#N]

### Step 1: tert-butyl 2-(1-(2,6-dioxopiperidin-3-yl)-3-methyl-2-oxo-2,3-dihydro-1H-benzo[d]imidazol-5-yl)-2,7-diazaspiro[3.5]nonane-7-carboxylate (118a)

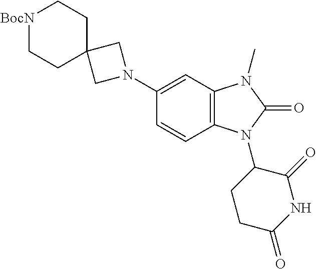 [SMILES: Cn1c(=O)n(C2CCC(=O)NC2=O)c2ccc(N3CC4(CCN(C(=O)OC(C)(C)C)CC4)C3)cc21]

Under nitrogen atmosphere, to a reaction flask were respectively added 3-(5-bromo-3-methyl-2-oxo-2,3-dihydro-1H-benzo[d]imidazol-1-yl)piperidine-2,6-dione (88a) (see WO 2020113233 for the synthetic method) (500 mg, 1.48 mmol), tert-butyl 2,7-diazaspiro[3.5]nonane-7-carboxylate (500 mg, 2.21 mmol), RuPhos-Pd-G3 (CAS: 1445085-77-7) (247 mg, 0.287 mmol) and RuPhos (138 mg, 0.296 mmol). The reaction system was subjected to nitrogen replacement three times, and toluene (10 mL) and 1 mol/L solution of LiHMDS in tetrahydrofuran (7.39 mL, 7.39 mmol) were added dropwise, respectively. The mixture was warmed to 80° C. and reacted for 4 h. The reaction system was cooled to room temperature, and under an ice bath, a saturated ammonium chloride solution (10 mL) was added to quench the reaction. The mixture was extracted with ethyl acetate (50 mL×3), and the organic phase was washed with 50 mL of saturated sodium chloride solution, dried over anhydrous sodium sulfate, and concentrated under reduced pressure. The crude product was separated and purified by silica gel column chromatography (ethyl acetate/petroleum ether (v/v)=2:1), to afford 118a (90 mg, yield: 13%).

### Step 2: 3-(3-methyl-2-oxo-5-(2,7-diazaspiro[3.5]nonan-2-yl)-2,3-dihydro-1H-benzo[d]imidazol-1-yl)piperidine-2,6-dione (118b) trifluoroacetate

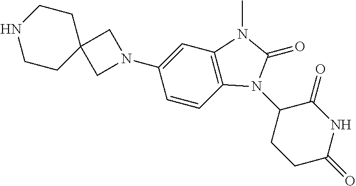 [SMILES: Cn1c(=O)n(C2CCC(=O)NC2=O)c2ccc(N3CC4(CCNCC4)C3)cc21]

118a (90 mg, 0.19 mmol) was dissolved in 2 mL of dichloromethane, and trifluoroacetic acid (1.53 g, 13.42 mmol) was added. The mixture was reacted at room temperature for 2 h. The reaction system was concentrated under reduced pressure, to afford crude 118b trifluoroacetate (90 mg).

### Step 3: tert-butyl 4-((2-(1-(2,6-dioxopiperidin-3-yl)-3-methyl-2-oxo-2,3-dihydro-1H-benzo[d]imidazol-5-yl)-2,7-diazaspiro[3.5]nonan-7-yl)methyl)piperidine-1-carboxylate (118c)

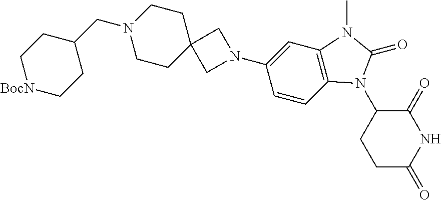 [SMILES: Cn1c(=O)n(C2CCC(=O)NC2=O)c2ccc(N3CC4(CCN(CC5CCN(C(=O)OC(C)(C)C)CC5)CC4)C3)cc21]

The above crude 118b trifluoroacetate (90 mg) was dissolved in 5 mL of tetrahydrofuran, and dimethyl sulfoxide (0.5 mL) and solid sodium bicarbonate (0.084 g, 1.0 mmol) were added. The mixture was stirred at room temperature for 0.5 h, and then tert-butyl 4-formylpiperidine-1-carboxylate (0.11 g, 0.55 mmol) and glacial acetic acid (0.5 mL) were added. The mixture was reacted at room temperature for 2 h, and sodium triacetoxyborohydride (0.11 g, 0.52 mmol) was added. The resulting mixture was reacted at room temperature for 16 h. The reaction liquid was quenched with a saturated sodium bicarbonate solution (10 mL), and extracted with ethyl acetate (20 mL×3). The organic phase was washed with 20 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 118c (70 mg, two-step yield from compound 118a: 63%).

### Step 4: 3-(3-methyl-2-oxo-5-(7-(piperidin-4-ylmethyl)-2,7-diazaspiro[3.5]nonan-2-yl)-2,3-dihydro-1H-benzo[d]imidazol-1-yl)piperidine-2,6-dione (118d) trifluoroacetate

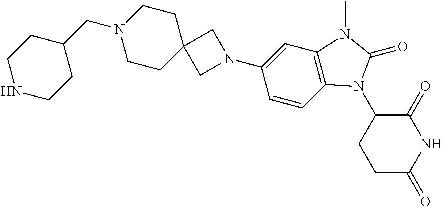 [SMILES: Cn1c(=O)n(C2CCC(=O)NC2=O)c2ccc(N3CC4(CCN(CC5CCNCC5)CC4)C3)cc21]

118c (70 mg, 0.12 mmol) was dissolved in 2 mL of dichloromethane, and trifluoroacetic acid (1.53 g, 13.42 mmol) was added. The mixture was reacted at room temperature for 2 h. The reaction system was concentrated under reduced pressure, to afford crude 118d trifluoroacetate (70 mg).

### Step 5: 3-chloro-5-(2-(4-((2-(4-((2-(1-(2,6-dioxopiperidin-3-yl)-3-methyl-2-oxo-2,3-dihydro-1H-benzo[d]imidazol-5-yl)-2,7-diazaspiro[3.5]nonan-7-yl)methyl)piperidin-1-yl)pyrimidin-4-yl)methoxy)phenyl)propan-2-yl)-2-ethoxybenzonitrile (Compound 118) trifluoroacetate

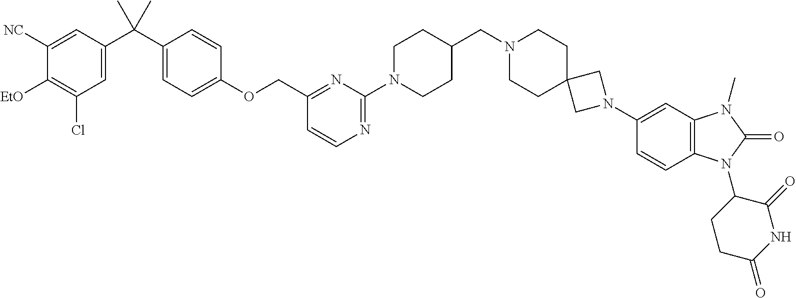 [SMILES: CCOc1c(Cl)cc(C(C)(C)c2ccc(OCc3ccnc(N4CCC(CN5CCC6(CC5)CN(c5ccc7c(c5)n(C)c(=O)n7C5CCC(=O)NC5=O)C6)CC4)n3)cc2)cc1C#N]

The above crude 118d trifluoroacetate (35 mg) was dissolved in 5 mL of DMF, and solid potassium bicarbonate (40 mg, 0.40 mmol) was added. The mixture was stirred at room temperature for 0.5 h, and then 3-chloro-5-(2-(4-((2-chloropyrimidin-4-yl)methoxy)phenyl)propan-2-yl)-2-ethoxybenzonitrile (18g) (44 mg, 0.10 mmol) was added. The resulting mixture was warmed to 80° C. and reacted for 4 h. The reaction liquid was cooled to room temperature, and water (10 mL) was added. The mixture was extracted with ethyl acetate (20 mL×3), and the organic phase was washed with 10 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 118 trifluoroacetate (24 mg).

¹H NMR (400 MHz, DMSO-d₆) δ 11.03 (s, 1H), 8.36 (d, 1H), 7.63-7.52 (m, 2H), 7.24-7.12 (m, 2H), 7.00-6.88 (m, 3H), 6.69 (d, 1H), 6.33-6.25 (m, 1H), 6.17-6.07 (m, 1H), 5.33-5.20 (m, 1H), 4.97 (s, 2H), 4.75-4.59 (m, 2H), 4.19 (q, 2H), 3.75-3.36 (m, 6H), 3.34-3.15 (m, 3H), 3.10-2.80 (m, 7H), 2.74-2.55 (m, 2H), 2.25-1.91 (m, 6H), 1.87-1.73 (m, 2H), 1.62 (s, 6H), 1.37 (t, 3H), 1.25-1.05 (m, 2H).

LCMS m/z=443.8 [M/2+1]⁺.

### Example 119

### 3-chloro-5-(2-(4-((2-(4-((1′-(4-(2,6-dioxopiperidin-3-yl)phenyl)-[4,4′-bipiperidin]-1-yl)methyl)piperidin-1-yl)pyrimidin-4-yl)methoxy)phenyl)propan-2-yl)-2-methoxybenzonitrile (Compound 119)

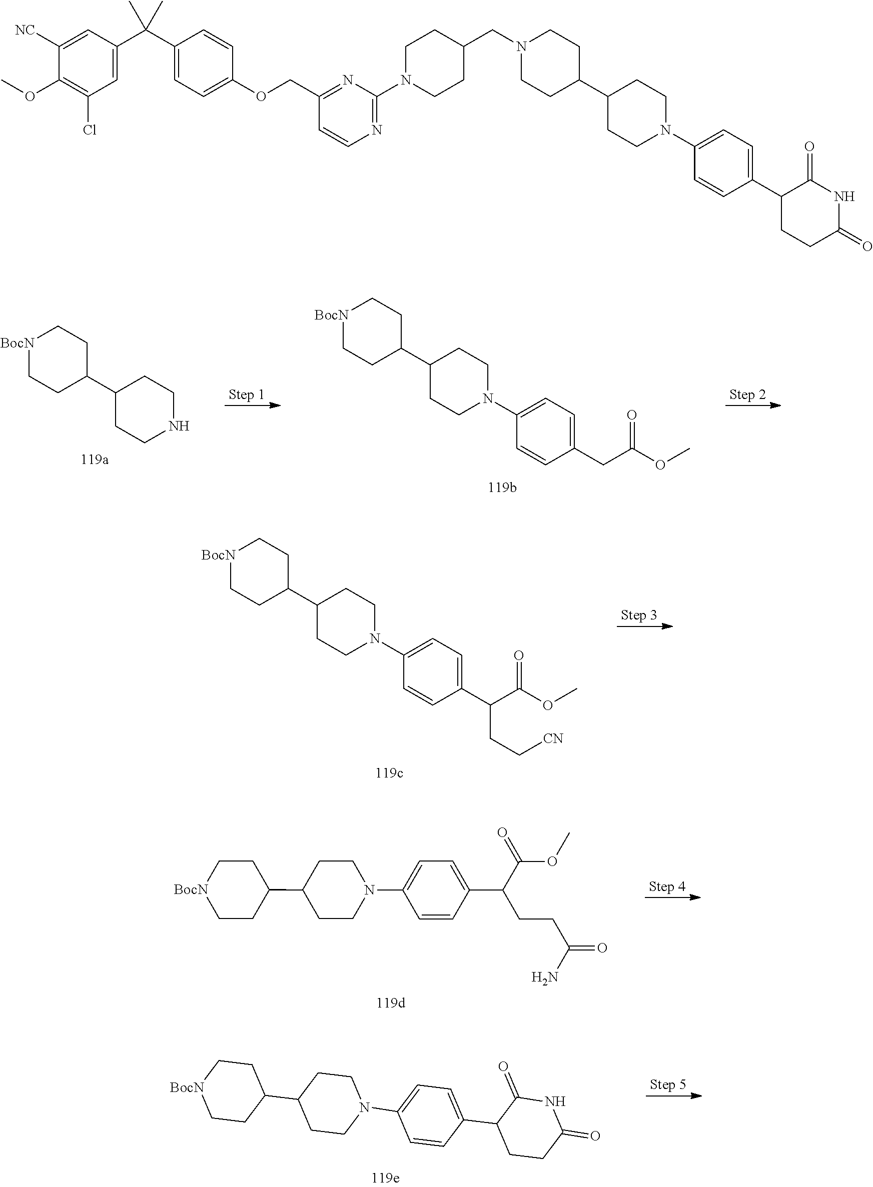 [SMILES: COc1c(Cl)cc(C(C)(C)c2ccc(OCc3ccnc(N4CCC(CN5CCC(C6CCN(c7ccc(C8CCC(=O)NC8=O)cc7)CC6)CC5)CC4)n3)cc2)cc1C#N ; CC(C)(C)OC(=O)N1CCC(C2CCNCC2)CC1 ; COC(=O)Cc1ccc(N2CCC(C3CCN(C(=O)OC(C)(C)C)CC3)CC2)cc1 ; COC(=O)C(CCC#N)c1ccc(N2CCC(C3CCN(C(=O)OC(C)(C)C)CC3)CC2)cc1 ; COC(=O)C(CCC(N)=O)c1ccc(N2CCC(C3CCN(C(=O)OC(C)(C)C)CC3)CC2)cc1 ; CC(C)(C)OC(=O)N1CCC(C2CCN(c3ccc(C4CCC(=O)NC4=O)cc3)CC2)CC1]

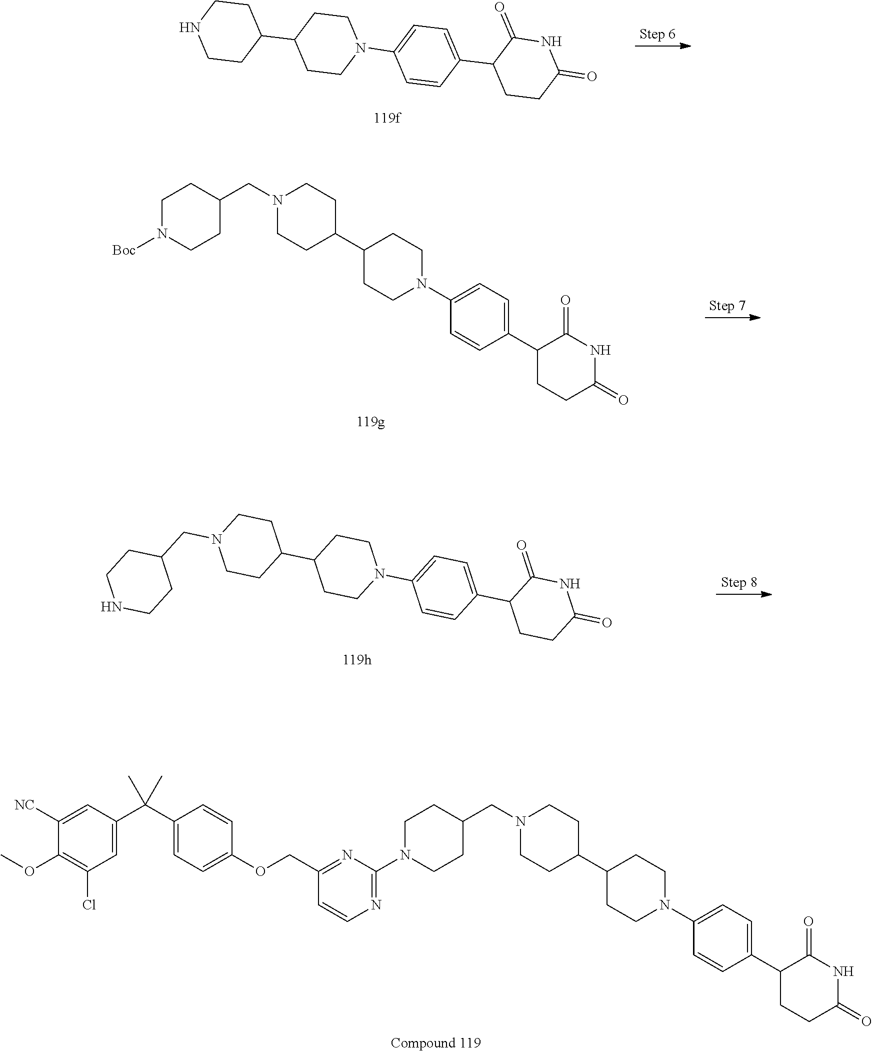 [SMILES: O=C1CCC(c2ccc(N3CCC(C4CCNCC4)CC3)cc2)C(=O)N1 ; CC(C)(C)OC(=O)N1CCC(CN2CCC(C3CCN(c4ccc(C5CCC(=O)NC5=O)cc4)CC3)CC2)CC1 ; O=C1CCC(c2ccc(N3CCC(C4CCN(CC5CCNCC5)CC4)CC3)cc2)C(=O)N1 ; COc1c(Cl)cc(C(C)(C)c2ccc(OCc3ccnc(N4CCC(CN5CCC(C6CCN(c7ccc(C8CCC(=O)NC8=O)cc7)CC6)CC5)CC4)n3)cc2)cc1C#N]

### Step 1: tert-butyl 1′-(4-(2-methoxy-2-oxoethyl)phenyl)-[4,4′-bipiperidine]-1-carboxylate (119b)

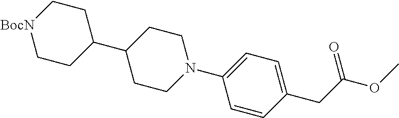 [SMILES: COC(=O)Cc1ccc(N2CCC(C3CCN(C(=O)OC(C)(C)C)CC3)CC2)cc1]

Under nitrogen protection, to a 250 mL single-necked flask were successively added tert-butyl [4,4′-bipiperidine]-1-carboxylate (119a) (5.0 g, 18.6 mmol), methyl p-bromophenylacetate (4.2 g, 18.3 mmol), cesium carbonate (12.09 g, 37.1 mmol), toluene (50.0 mL) and RuPhos-Pd-G2 (CAS: 1375325-68-0) (1.3 g, 1.67 mmol). The mixture was subjected to nitrogen replacement three times, warmed to 120° C. and reacted for 20 h. The reaction liquid was cooled to room temperature, and filtered by suction under reduced pressure. The filtrate was concentrated under reduced pressure, and the crude was separated and purified by silica gel column chromatography (petroleum ether/ethyl acetate (v/v)=2:1-3:2), to afford 119b (5.2 g, yield: 68%).

LCMS m/z=417.2 [M+1]⁺

### Step 2: tert-butyl 1′-(4-(4-cyano-1-methoxy-1-oxobutan-2-yl)phenyl)-[4,4′-bipiperidine]-1-carboxylate (119c)

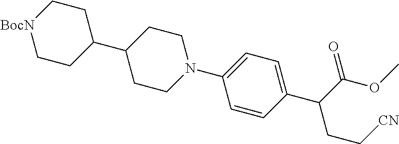 [SMILES: COC(=O)C(CCC#N)c1ccc(N2CCC(C3CCN(C(=O)OC(C)(C)C)CC3)CC2)cc1]

119b (2.3 g, 5.52 mmol) was dissolved in 30 mL of toluene, and acrylonitrile (439 mg, 8.3 mmol) and a 40% solution of N,N,N-trimethyl-1-phenylmethanamine hydroxide in methanol (CAS: 100-85-6) (114.8 mg (solution mass)) were added. The mixture was reacted at room temperature for 48 h. The reaction system was concentrated under reduced pressure, and 150 mL of water was added. The mixture was extracted with dichloromethane (60 mL×3), and the organic phase was washed with a saturated sodium chloride solution (50 mL×3), dried over anhydrous sodium sulfate, and concentrated under reduced pressure. The crude product was then separated and purified by silica gel column chromatography (petroleum ether/ethyl acetate (v/v)=1:2-1:3), to afford 119c (0.32 g, yield: 12%).

LCMS m/z=470.2 [M+1]⁺

### Step 3: tert-butyl 1′-(4-(5-amino-1-methoxy-1,5-dioxopentan-2-yl)phenyl)-[4,4′-bipiperidine]-1-carboxylate (119d)

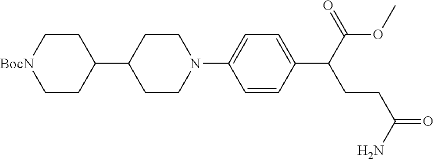 [SMILES: COC(=O)C(CCC(N)=O)c1ccc(N2CCC(C3CCN(C(=O)OC(C)(C)C)CC3)CC2)cc1]

119c (320.0 mg, 0.68 mmol) was dissolved in 15 mL of toluene, and indium trichloride tetrahydrate (79.6 mg, 0.272 mmol) and acetaldehyde oxime (108.2 mg, 1.83 mmol) were added. The mixture was warmed to 130° C. and reacted for 5 h. The reaction liquid was cooled to room temperature and concentrated under reduced pressure, and 100 mL of water was added. The mixture was extracted with ethyl acetate (50 mL×3), and the organic phase was washed with a saturated sodium chloride solution (60 mL×3), dried over anhydrous sodium sulfate, and concentrated under reduced pressure. The crude product was then separated and purified by silica gel column chromatography (petroleum ether/ethyl acetate (v/v)=1:1-0:1), to afford 119d (160.0 mg, yield: 48%).

LCMS m/z=488.3 [M+1]⁺

### Step 4: tert-butyl 1′-(4-(2,6-dioxopiperidin-3-yl)phenyl)-[4,4′-bipiperidine]-1-carboxylate (119e)

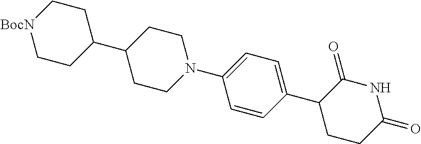 [SMILES: CC(C)(C)OC(=O)N1CCC(C2CCN(c3ccc(C4CCC(=O)NC4=O)cc3)CC2)CC1]

119d (160.0 mg, 0.328 mmol) was dissolved in 10.0 mL of acetonitrile, and a 40% solution of N,N,N-trimethyl-1-phenylmethanamine hydroxide in methanol (CAS: 100-85-6) (205.4 mg (solution mass)) was added. The mixture was warmed to 60° C. and reacted for 1.0 h. The reaction liquid was cooled to room temperature and concentrated under reduced pressure, and 150 mL of water was added. The mixture was extracted with dichloromethane (50 mL×3), and the organic phase was washed with a saturated sodium chloride solution (90.0 mL×3), dried over anhydrous sodium sulfate, and concentrated under reduced pressure. The crude product was then separated and purified by silica gel column chromatography (petroleum ether/ethyl acetate (v/v)=1:1-0:1), to afford 119e (115 mg, yield: 77%).

LCMS m/z=456.2 [M+1]⁺

### Step 5: 3-(4-([4,4′-bipiperidin]-1-yl)phenyl)piperidine-2,6-dione (119f) trifluoroacetate

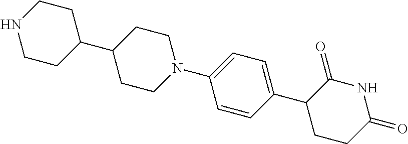 [SMILES: O=C1CCC(c2ccc(N3CCC(C4CCNCC4)CC3)cc2)C(=O)N1]

119e (115 mg, 0.252 mmol) was dissolved in 8 mL of dichloromethane, and trifluoroacetic acid (4 mL) was added. The mixture was reacted at room temperature for 1.0 h. The reaction liquid was concentrated under reduced pressure, to afford crude 119f trifluoroacetate (90.0 mg).

LCMS m/z=356.2 [M+1]⁺

### Step 6: tert-butyl 4-((1′-(4-(2,6-dioxopiperidin-3-yl)phenyl)-[4,4′-bipiperidin]-1-yl)methyl) piperidine-1-carboxylate (119g)

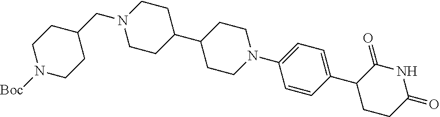 [SMILES: CC(C)(C)OC(=O)N1CCC(CN2CCC(C3CCN(c4ccc(C5CCC(=O)NC5=O)cc4)CC3)CC2)CC1]

The above crude 119f trifluoroacetate (90.0 mg) was dissolved in a mixed solvent of 4.0 mL of dichloromethane, 4.0 mL of tetrahydrofuran and 1.0 mL of dimethyl sulfoxide, and solid sodium bicarbonate (108.0 mg, 1.29 mmol) was added. The mixture was stirred at room temperature for 20 min, and then tert-butyl 4-formylpiperidine-1-carboxylate (107.0 mg, 0.502 mmol) was added. Acetic acid was added dropwise to adjust the reaction liquid to pH 6, and the mixture was stirred at room temperature for 1.0 h. Sodium triacetoxyborohydride (106.0 mg, 0.50 mmol) was then added, and the resulting mixture was reacted at room temperature for 4.0 h. To the reaction liquid was added a saturated sodium bicarbonate solution (2.0 mL) to quench the reaction, and the mixture was extracted with dichloromethane (30 mL×3). The organic phase was washed with a saturated sodium chloride solution (50 mL×3), dried over anhydrous sodium sulfate, and concentrated under reduced pressure. The crude product was then separated and purified by silica gel column chromatography (DCM/MeOH (v/v)=19:1-10:1), to afford 119g (95.0 mg, two-step yield from compound 119e: 68%).

LCMS m/z=553.2 [M+1]⁺

### Step 7: 3-(4-(1′-(piperidin-4-ylmethyl)-[4,4′-bipiperidin]-1-yl)phenyl)piperidine-2,6-dione (119h) trifluoroacetate

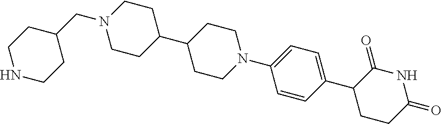 [SMILES: O=C1CCC(c2ccc(N3CCC(C4CCN(CC5CCNCC5)CC4)CC3)cc2)C(=O)N1]

119g (95.0 mg, 0.172 mmol) was dissolved in 10.0 mL of dichloromethane, and trifluoroacetic acid (4.0 mL) was added. The mixture was reacted at room temperature for 1.0 h. The reaction liquid was concentrated under reduced pressure, to afford crude 119h trifluoroacetate (85 mg).

LCMS m/z=453.2 [M+1]⁺

### Step 8: 3-chloro-5-(2-(4-((2-(4-((1′-(4-(2,6-dioxopiperidin-3-yl)phenyl)-[4,4′-bipiperidin]-1-yl)methyl)piperidin-1-yl)pyrimidin-4-yl)methoxy)phenyl)propan-2-yl)-2-methoxybenzonitrile (compound 119)

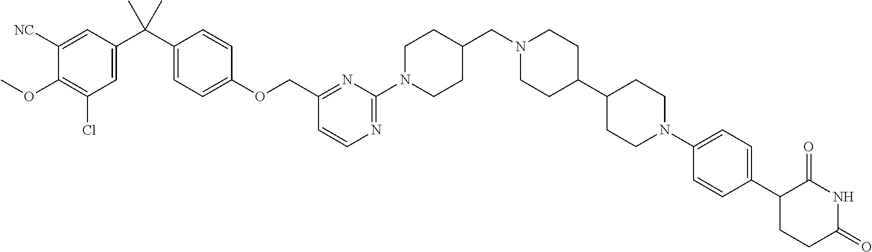 [SMILES: COc1c(Cl)cc(C(C)(C)c2ccc(OCc3ccnc(N4CCC(CN5CCC(C6CCN(c7ccc(C8CCC(=O)NC8=O)cc7)CC6)CC5)CC4)n3)cc2)cc1C#N]

The above crude 119h trifluoroacetate (85.0 mg) was dissolved in 5.0 mL of DMF, and solid potassium bicarbonate (150 mg, 1.5 mmol) and 3-chloro-5-(2-(4-((2-chloropyrimidin-4-yl)methoxy)phenyl)propan-2-yl)-2-methoxybenzonitrile (92e) (64.2 mg, 0.15 mmol) were successively added. The mixture was warmed to 70° C. and reacted for 4.0 h. The reaction liquid was cooled to room temperature, and water (60.0 mL) was added. The mixture was extracted with dichloromethane (30 mL×3), and the organic phase was washed with a saturated sodium chloride solution (50 mL×3), dried over anhydrous sodium sulfate, and concentrated under reduced pressure. The crude product was then separated and purified by a thick preparative plate (developing agent: DCM/MeOH (v/v)=15:1), to afford compound 119 (60.0 mg, two-step yield from compound 119g: 41%).

¹H NMR (400 MHz, CDCl₃) δ 8.29 (d, 1H), 8.00-7.85 (m, 1H), 7.45-7.29 (m, 2H), 7.14-7.00 (m, 4H), 7.00-6.83 (m, 4H), 6.66 (d, 1H), 4.94 (s, 2H), 4.85-4.65 (m, 2H), 4.04 (s, 3H), 3.78-3.65 (m, 3H), 3.04-2.55 (m, 8H), 2.32-2.09 (m, 4H), 1.96-1.55 (m, 14H), 1.50-1.00 (m, 9H).

LCMS m/z=844.1 [M+1]⁺

### Example 120

### 3-chloro-5-(2-(4-((2-(4-((1′-(4-(2,4-dioxotetrahydropyrimidin-1(2H)-yl)phenyl)-[4,4′-bipiperidin]-1-yl)methyl)piperidin-1-yl)pyrimidin-4-yl)methoxy)phenyl)propan-2-yl)-2-methoxybenzonitrile (compound 120)

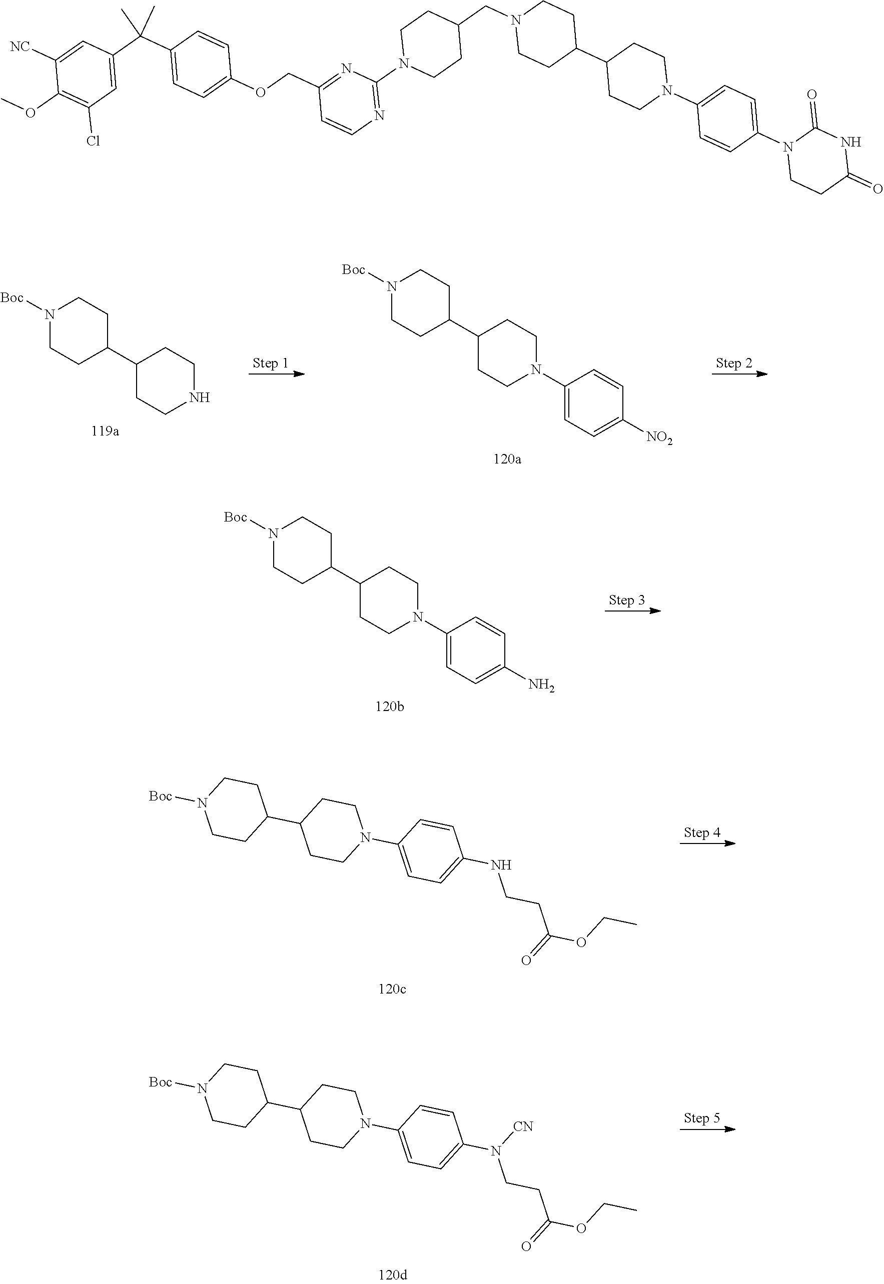 [SMILES: COc1c(Cl)cc(C(C)(C)c2ccc(OCc3ccnc(N4CCC(CN5CCC(C6CCN(c7ccc(N8CCC(=O)NC8=O)cc7)CC6)CC5)CC4)n3)cc2)cc1C#N ; CC(C)(C)OC(=O)N1CCC(C2CCNCC2)CC1 ; CC(C)(C)OC(=O)N1CCC(C2CCN(c3ccc([N+](=O)[O-])cc3)CC2)CC1 ; CC(C)(C)OC(=O)N1CCC(C2CCN(c3ccc(N)cc3)CC2)CC1 ; CCOC(=O)CCNc1ccc(N2CCC(C3CCN(C(=O)OC(C)(C)C)CC3)CC2)cc1 ; CCOC(=O)CCN(C#N)c1ccc(N2CCC(C3CCN(C(=O)OC(C)(C)C)CC3)CC2)cc1]

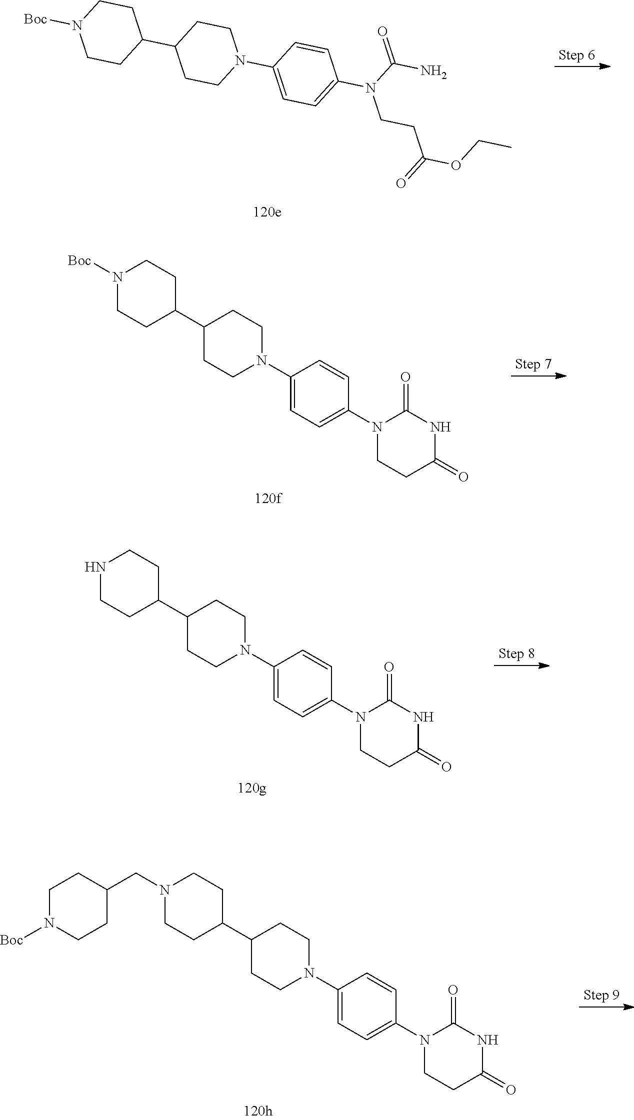 [SMILES: CCOC(=O)CCN(C(N)=O)c1ccc(N2CCC(C3CCN(C(=O)OC(C)(C)C)CC3)CC2)cc1 ; CC(C)(C)OC(=O)N1CCC(C2CCN(c3ccc(N4CCC(=O)NC4=O)cc3)CC2)CC1 ; O=C1CCN(c2ccc(N3CCC(C4CCNCC4)CC3)cc2)C(=O)N1 ; CC(C)(C)OC(=O)N1CCC(CN2CCC(C3CCN(c4ccc(N5CCC(=O)NC5=O)cc4)CC3)CC2)CC1]

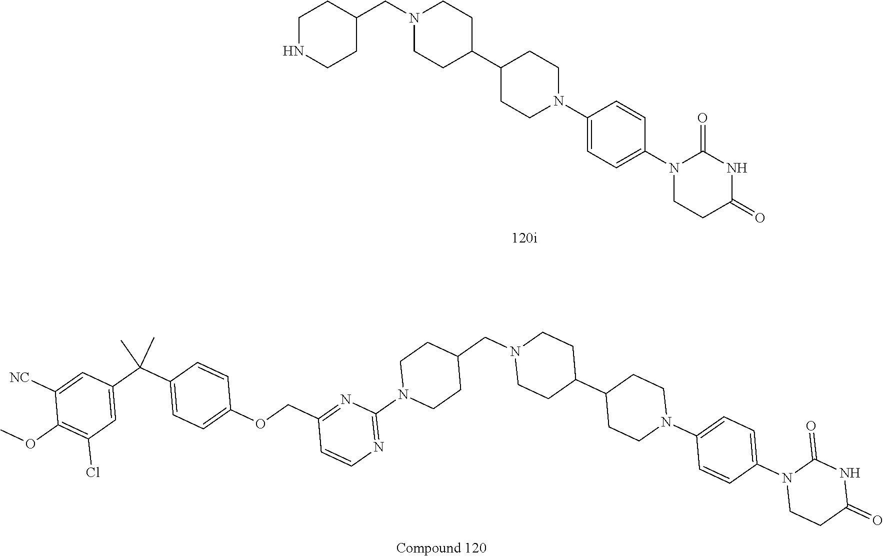 [SMILES: O=C1CCN(c2ccc(N3CCC(C4CCN(CC5CCNCC5)CC4)CC3)cc2)C(=O)N1 ; COc1c(Cl)cc(C(C)(C)c2ccc(OCc3ccnc(N4CCC(CN5CCC(C6CCN(c7ccc(N8CCC(=O)NC8=O)cc7)CC6)CC5)CC4)n3)cc2)cc1C#N]

### Step 2: tert-butyl 1′-(4-nitrophenyl)-[4,4′-bipiperidine]-1-carboxylate (120a)

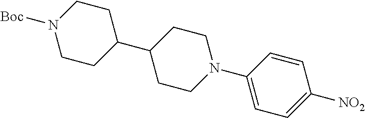 [SMILES: CC(C)(C)OC(=O)N1CCC(C2CCN(c3ccc([N+](=O)[O-])cc3)CC2)CC1]

Under nitrogen protection, to a 250 mL single-necked flask were successively added tert-butyl [4,4′-bipiperidine]-1-carboxylate (119a) (5.0 g, 18.6 mmol), p-fluoronitrobenzene (2.63 g, 18.6 mmol), potassium carbonate (7.7 g, 55.7 mmol) and DMF (60.0 mL), and the mixture was warmed to 50° C. and reacted for 4 h. The reaction liquid was cooled to room temperature and slowly added dropwise to 600.0 mL of ice water to precipitate out a solid. The solid was filtered by suction, and the filter cake was dried by blowing at a temperature of 50′C, to afford crude 120a (6.5 g).

LCMS m/z=390.2 [M+1]⁺

### Step 2: tert-butyl 1′-(4-aminophenyl)-[4,4′-bipiperidine]-1-carboxylate (120b)

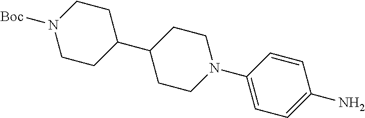 [SMILES: CC(C)(C)OC(=O)N1CCC(C2CCN(c3ccc(N)cc3)CC2)CC1]

To a 500 mL single-necked reaction flask were respectively added the above crude 120a (6.5 g), MeOH (130 mL), DCM (40 mL) and 10% palladium on carbon (650 mg), and the mixture was reacted at room temperature under hydrogen atmosphere for 16 h. The reaction system was filtered by suction, and the filtrate was concentrated under reduced pressure, to afford crude 120b (4.8 g).

LCMS m/z=360.2 [M+1]⁺

### Step 3: tert-butyl 1′-(4-((3-ethoxy-3-oxopropyl)amino)phenyl)-[4,4′-bipiperidine]-1-carboxylate (120c)

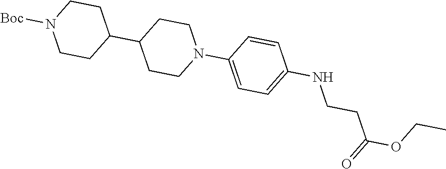 [SMILES: CCOC(=O)CCNc1ccc(N2CCC(C3CCN(C(=O)OC(C)(C)C)CC3)CC2)cc1]

The above crude 120b (3.8 g) was dissolved in 90.0 mL of 1,4-dioxane, and ethyl acrylate (2.11 g, 21.1 mmol) and DBU lactate (5.09 g, 21.00 mmol) were added. The mixture was warmed to 90° C. and reacted for 60 h. The reaction liquid was cooled to room temperature and concentrated under reduced pressure, and water (300 mL) was added. The mixture was extracted with ethyl acetate (160 mL×3), and the organic phase was washed with 300 mL of saturated sodium chloride solution, and dried over anhydrous sodium sulfate. The crude product was separated and purified by silica gel column chromatography (petroleum ether/ethyl acetate (v/v)=1:1-2:3), to afford 120c (1.8 g, three-step yield from compound 119a: 27%).

LCMS m/z=460.3 [M+1]⁺

### Step 4: tert-butyl 1′-(4-(N-(3-ethoxy-3-oxopropyl)cyanamido)phenyl)-[4,4′-bipiperidine]-1-carboxylate (120d)

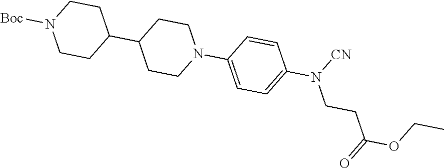 [SMILES: CCOC(=O)CCN(C#N)c1ccc(N2CCC(C3CCN(C(=O)OC(C)(C)C)CC3)CC2)cc1]

120c (1.8 g, 3.92 mmol) was dissolved in 35 mL of tetrahydrofuran, and solid sodium bicarbonate (3.37 g, 40.12 mmol) and cyanogen bromide (823.5 mg, 7.8 mmol) were added. The mixture was reacted at room temperature for 5.0 h. To the reaction liquid was added water (100.0 mL), and the mixture was extracted with ethyl acetate (60 mL×3). The organic phase was washed with 200 mL of saturated sodium chloride solution, and dried over anhydrous sodium sulfate. The crude product was separated and purified by silica gel column chromatography (petroleum ether/ethyl acetate (v/v)=1:2-1:3), to afford 120d (1.4 g, yield: 74%).

LCMS m/z=485.3 [M+1]⁺

### Step 5: tert-butyl 1′-(4-(1-(3-ethoxy-3-oxopropyl)ureido)phenyl)-[4,4′-bipiperidine]-1-carboxylate (120e)

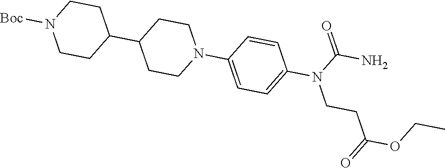 [SMILES: CCOC(=O)CCN(C(N)=O)c1ccc(N2CCC(C3CCN(C(=O)OC(C)(C)C)CC3)CC2)cc1]

120d (1.4 g, 2.89 mmol) was dissolved in 30.0 mL of toluene, and indium trichloride tetrahydrate (339 mg, 1.16 mmol) and acetaldehyde oxime (511.5 mg, 8.66 mmol) were successively added. The mixture was warmed to 130° C. and reacted for 6.0 h. The reaction liquid was cooled to room temperature and concentrated under reduced pressure, and water (200.0 mL) was added. The mixture was extracted with ethyl acetate (50 mL×3), and the organic phase was washed with a saturated sodium chloride solution (100.0 mL×3), dried over anhydrous sodium sulfate, and concentrated under reduced pressure. The crude product was then separated and purified by silica gel column chromatography (petroleum ether/ethyl acetate (v/v)=1:1-0:1), to afford 120e (0.9 g, yield: 62%).

LCMS m/z=503.3 [M+1]⁺

### Step 6: tert-butyl 1′-(4-(2,4-dioxotetrahydropyrimidin-1(2H)-yl)phenyl)-[4,4′-bipiperidine]-1-carboxylate (120f)

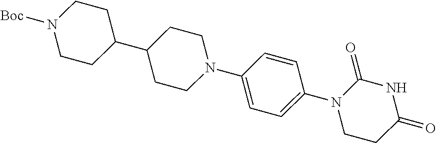 [SMILES: CC(C)(C)OC(=O)N1CCC(C2CCN(c3ccc(N4CCC(=O)NC4=O)cc3)CC2)CC1]

120e (0.9 g, 1.79 mmol) was dissolved in 20.0 mL of acetonitrile, and a 40% solution of N,N,N-trimethyl-1-phenylmethanamine hydroxide in methanol (CAS: 100-85-6) (1.12 g (solution mass)) was added. The mixture was warmed to 60° C. and reacted for 1.0 h. The reaction liquid was cooled to room temperature and concentrated under reduced pressure, and water (150.0 mL) was added. The mixture was extracted with dichloromethane (50 mL×3), and the organic phase was washed with a saturated sodium chloride solution (90.0 mL×3), dried over anhydrous sodium sulfate, and concentrated under reduced pressure. The crude product was then separated and purified by silica gel column chromatography (petroleum ether/ethyl acetate (v/v)=1:1-0:1), to afford 120f (0.57 g, yield: 70%).

LCMS m/z=457.2 [M+1]⁺

### Step 7: 1-(4-([4,4′-bipiperidin]-1-yl)phenyl)dihydropyrimidine-2,4(1H,3H)-dione (120g) trifluoroacetate

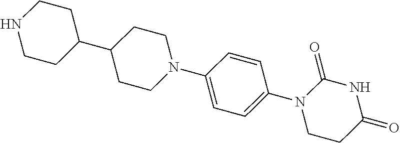 [SMILES: O=C1CCN(c2ccc(N3CCC(C4CCNCC4)CC3)cc2)C(=O)N1]

120f (140 mg, 0.307 mmol) was dissolved in 10.0 mL of dichloromethane, and trifluoroacetic acid (4.0 mL) was added. The mixture was reacted at room temperature for 1.0 h. The reaction liquid was concentrated under reduced pressure, to afford crude 120g trifluoroacetate (130 mg).

LCMS m/z=357.2 [M+1]⁺

### Step 8: tert-butyl 4-((1′-(4-(2,4-dioxotetrahydropyrimidin-1(2H)-yl)phenyl)-[4,4′-bipiperidin]-1-yl)methyl)piperidine-1-carboxylate (120h)

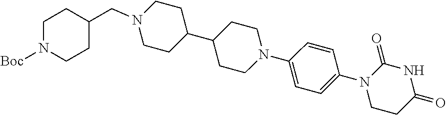 [SMILES: CC(C)(C)OC(=O)N1CCC(CN2CCC(C3CCN(c4ccc(N5CCC(=O)NC5=O)cc4)CC3)CC2)CC1]

The above crude 120g trifluoroacetate (130 mg) was dissolved in a mixed solvent of 4.0 mL of dichloromethane, 4.0 mL of tetrahydrofuran and 1.0 mL of dimethyl sulfoxide, and solid sodium bicarbonate (132.0 mg, 1.57 mmol) was added. The mixture was reacted at room temperature for 20.0 min, and then tert-butyl 4-formylpiperidine-1-carboxylate (130.0 mg, 0.610 mmol) was added. Acetic acid was added dropwise to adjust the solution to pH 6, and the mixture was stirred at room temperature for 1.0 h. Sodium triacetoxyborohydride (129.0 mg, 0.609 mmol) was then added, and the resulting mixture was reacted at room temperature for 4.0 h. To the reaction liquid was added a saturated sodium bicarbonate solution (2.0 mL) to quench the reaction, and the mixture was extracted with dichloromethane (30 mL×3). The organic phase was washed with a saturated sodium chloride solution (50 mL×3), dried over anhydrous sodium sulfate, and concentrated under reduced pressure. The crude product was then separated and purified by silica gel column chromatography (DCM/MeOH (v/v)=19:1-10:1), to afford 120h (120.0 mg, two-step yield from compound 120f: 71%).

LCMS m/z=554.3 [M+1]⁺

### Step 9:1-(4-(1′-(piperidin-4-ylmethyl)-[4,4′-bipiperidin]-1-yl)phenyl)dihydropyrimidine-2,4(1H,3H)-dione (120i) trifluoroacetate

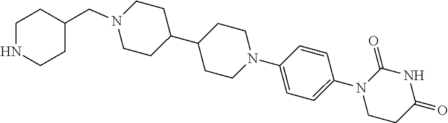 [SMILES: O=C1CCN(c2ccc(N3CCC(C4CCN(CC5CCNCC5)CC4)CC3)cc2)C(=O)N1]

120h (120 mg, 0.217 mmol) was dissolved in 10.0 mL of dichloromethane, and trifluoroacetic acid (4.0 mL) was added. The mixture was reacted at room temperature for 1.0 h. The reaction liquid was concentrated under reduced pressure, to afford crude 120i trifluoroacetate (80 mg).

LCMS m/z=454.3 [M+1]⁺

### Step 10: 3-chloro-5-(2-(4-((2-(4-((1′-(4-(2,4-dioxotetrahydropyrimidin-1(2H)-yl)phenyl)-[4,4′-bipiperidin]-1-yl)methyl)piperidin-1-yl)pyrimidin-4-yl)methoxy)phenyl)propan-2-yl)-2-methoxybenzonitrile (Compound 120)

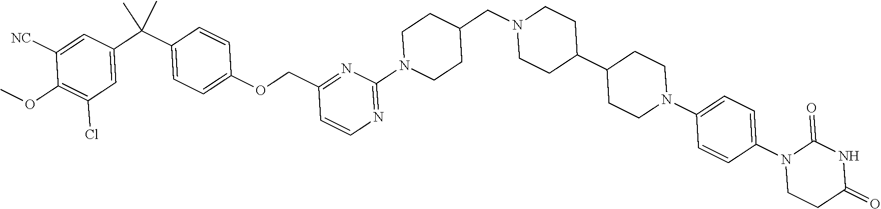 [SMILES: COc1c(Cl)cc(C(C)(C)c2ccc(OCc3ccnc(N4CCC(CN5CCC(C6CCN(c7ccc(N8CCC(=O)NC8=O)cc7)CC6)CC5)CC4)n3)cc2)cc1C#N]

The above crude 120i trifluoroacetate (80.0 mg) was dissolved in 6.0 mL of DMF, and solid potassium bicarbonate (140 mg, 1.4 mmol) and 3-chloro-5-(2-(4-((2-chloropyrimidin-4-yl)methoxy)phenyl)propan-2-yl)-2-methoxybenzonitrile (92e) (60.0 mg, 0.14 mmol) were successively added. The mixture was warmed to 70° C. and reacted for 4.0 h. The reaction liquid was cooled to room temperature, and water (60.0 mL) was added. The mixture was extracted with dichloromethane (30 mL×3), and the organic phase was washed with a saturated sodium chloride solution (50 mL×3), dried over anhydrous sodium sulfate, and concentrated under reduced pressure. The crude product was then separated and purified by a thick preparative plate (DCM/MeOH (v/v)=15:1), to afford compound 120 (60.0 mg, two-step yield from compound 120h: 33%).

¹H NMR (400 MHz, DMSO-d₆) δ 10.22 (s, 1H), 8.32 (d, 1H), 7.63-7.59 (m, 1H), 7.57-7.53 (m, 1H), 7.23-7.07 (m, 4H), 6.99-6.85 (m, 4H), 6.63 (d, 1H), 4.96 (s, 2H), 4.70-4.56 (m, 2H), 3.95 (s, 3H), 3.75-3.65 (m, 4H), 2.95-2.75 (m, 4H), 2.73-2.52 (m, 4H), 2.20-2.02 (m, 2H), 1.90-1.55 (m, 15H), 1.35-0.90 (m, 8H).

LCMS m/z=845.4 [M+1]⁺

### Example 121

### 3-chloro-5-(2-(4-((2-(7-(4-(1-(2,6-dioxopiperidin-3-yl)-3-methyl-2-oxo-2,3-dihydro-1H-benzo[d]imidazol-5-yl)piperidin-1-yl)-2-azaspiro[3.5]nonan-2-yl)pyrimidin-4-yl)methoxy)phenyl)propan-2-yl)-2-ethoxybenzonitrile (Compound 121) trifluoroacetate

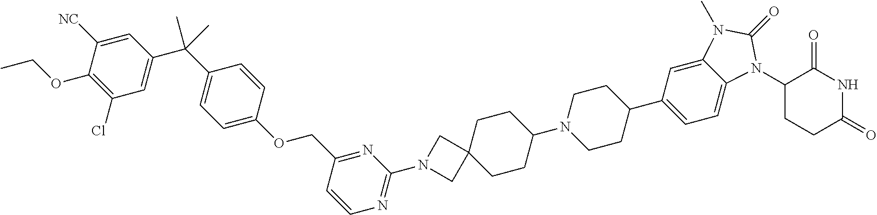 [SMILES: CCOc1c(Cl)cc(C(C)(C)c2ccc(OCc3ccnc(N4CC5(CCC(N6CCC(c7ccc8c(c7)n(C)c(=O)n8C7CCC(=O)NC7=O)CC6)CC5)C4)n3)cc2)cc1C#N]

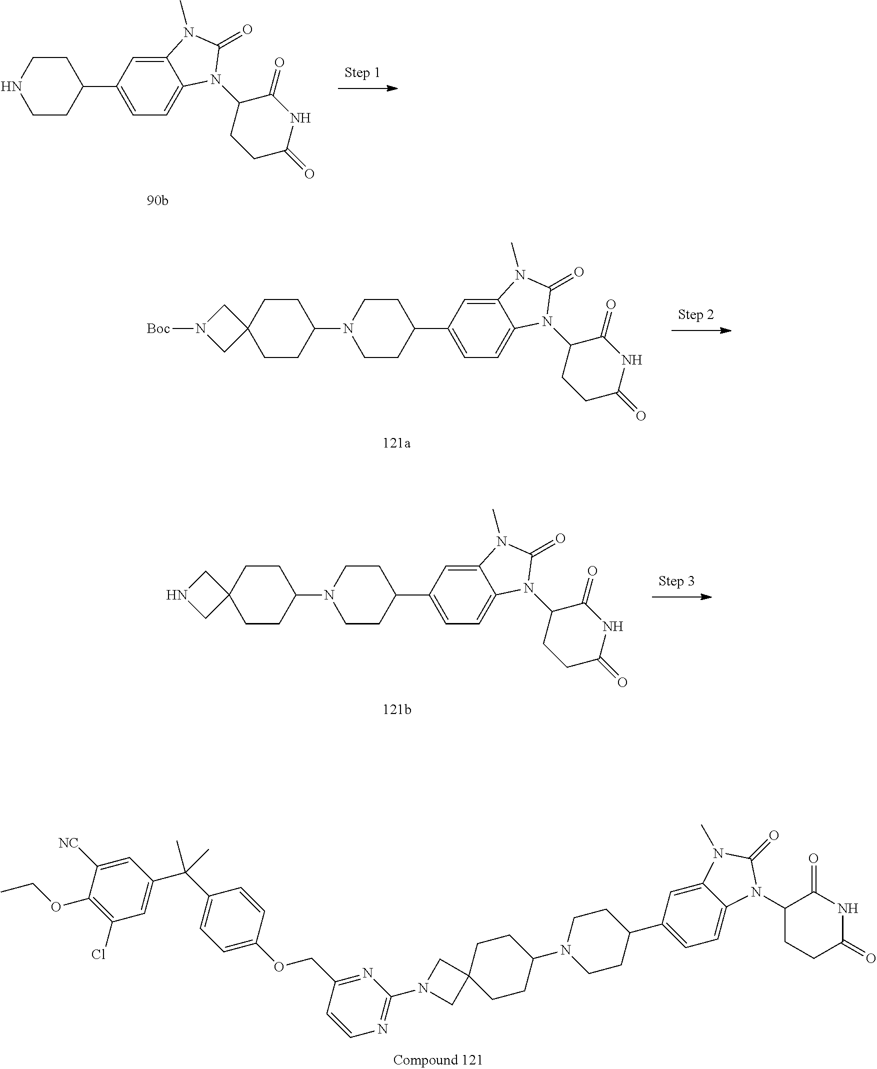 [SMILES: Cn1c(=O)n(C2CCC(=O)NC2=O)c2ccc(C3CCNCC3)cc21 ; Cn1c(=O)n(C2CCC(=O)NC2=O)c2ccc(C3CCN(C4CCC5(CC4)CN(C(=O)OC(C)(C)C)C5)CC3)cc21 ; Cn1c(=O)n(C2CCC(=O)NC2=O)c2ccc(C3CCN(C4CCC5(CC4)CNC5)CC3)cc21 ; CCOc1c(Cl)cc(C(C)(C)c2ccc(OCc3ccnc(N4CC5(CCC(N6CCC(c7ccc8c(c7)n(C)c(=O)n8C7CCC(=O)NC7=O)CC6)CC5)C4)n3)cc2)cc1C#N]

### Step 1: tert-butyl 7-(4-(1-(2,6-dioxopiperidin-3-yl)-3-methyl-2-oxo-2,3-dihydro-1H-benzo[d]imidazol-5-yl)piperidin-1-yl)-2-azaspiro[3.5]nonane-2-carboxylate (121a)

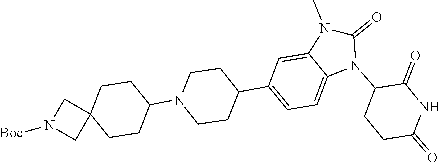 [SMILES: Cn1c(=O)n(C2CCC(=O)NC2=O)c2ccc(C3CCN(C4CCC5(CC4)CN(C(=O)OC(C)(C)C)C5)CC3)cc21]

The above crude 3-(3-methyl-2-oxo-5-(piperidin-4-yl)-2,3-dihydro-1H-benzo[d]imidazol-1-yl)piperidine-2,6-dione (90b) trifluoroacetate (2.0 g) was dissolved in 20 mL of tetrahydrofuran, and dimethyl sulfoxide (2 mL) and solid sodium bicarbonate (1.26 g, 15 mmol) were added. The mixture was stirred at room temperature for 0.5 h, and then filtered by suction. To the filtrate were added tert-butyl 7-oxo-2-azaspiro[3.5]nonane-carboxylate (2.39 g, 10 mmol) and glacial acetic acid (0.5 mL). The mixture was stirred at room temperature for 0.5 h, and then sodium triacetoxyborohydride (2.12 g, 10 mmol) was added. The resulting mixture was reacted at room temperature for 16 h. The reaction liquid was quenched with a sodium bicarbonate solution (10 mL), and extracted with ethyl acetate (30 mL×3). The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 121a (2.0 g, two-step yield from compound 90a: 94%).

### Step 2: 3-(5-(1-(2-azaspiro[3.5]nonan-7-yl)piperidin-4-yl)-3-methyl-2-oxo-2,3-dihydro-1H-benzo[d]imidazol-1-yl)piperidine-2,6-dione (121b) trifluoroacetate

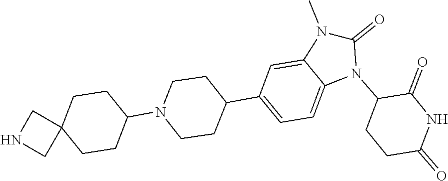 [SMILES: Cn1c(=O)n(C2CCC(=O)NC2=O)c2ccc(C3CCN(C4CCC5(CC4)CNC5)CC3)cc21]

To a reaction flask were successively added 121a (2.0 g, 3.5 mmol), trifluoroacetic acid (20 mL) and dichloromethane (20 mL), and the mixture was reacted at room temperature for 2 h. The reaction liquid was concentrated under reduced pressure, to afford crude 3-(5-(1-(2-azaspiro[3.5]nonan-7-yl)piperidin-4-yl)-3-methyl-2-oxo-2,3-dihydro-1H-benzo[d]imidazol-1-yl)piperidine-2,6-dione (121b) trifluoroacetate (2.0 g).

### Step 3: 3-chloro-5-(2-(4-((2-(7-(4-(1-(2,6-dioxopiperidin-3-yl)-3-methyl-2-oxo-2,3-dihydro-1H-benzo[d]imidazol-5-yl)piperidin-1-yl)-2-azaspiro[3.5]nonan-2-yl)pyrimidin-4-yl)methoxy)phenyl)propan-2-yl)-2-ethoxybenzonitrile (Compound 121) trifluoroacetate

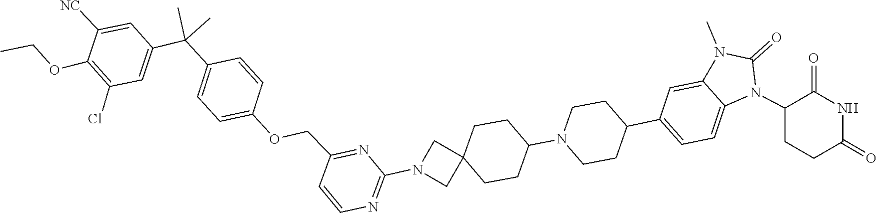 [SMILES: CCOc1c(Cl)cc(C(C)(C)c2ccc(OCc3ccnc(N4CC5(CCC(N6CCC(c7ccc8c(c7)n(C)c(=O)n8C7CCC(=O)NC7=O)CC6)CC5)C4)n3)cc2)cc1C#N]

The above crude 121b trifluoroacetate (2.00 g) was dissolved in 20 mL of DMF, and solid potassium bicarbonate (3.0 g, 30 mmol) was added. The mixture was stirred at room temperature for 0.5 h, and then 3-chloro-5-(2-(4-((2-chloropyrimidin-4-yl)methoxy)phenyl)propan-2-yl)-2-ethoxybenzonitrile (18g) (2.00 g, 4.52 mmol) was added. The resulting mixture was warmed to 80° C. and reacted for 4 h. The reaction liquid was cooled to room temperature, and water (20 mL) was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 121 trifluoroacetate (0.8 g).

¹H NMR (400 MHz, DMSO-d₆) δ 11.08 (s, 1H), 8.34 (d, 1H), 7.63-7.52 (m, 2H), 7.24-7.14 (m, 2H), 7.12-6.86 (m, 5H), 6.75 (d, 1H), 5.36 (dd, 1H), 4.97 (s, 2H), 4.19 (q, 2H), 3.81 (s, 2H), 3.73 (s, 2H), 3.62-3.47 (m, 2H), 3.35 (s, 3H), 3.32-3.06 (m, 3H), 3.00-2.83 (m, 2H), 2.78-2.56 (m, 2H), 2.20-1.85 (m, 9H), 1.75-1.47 (m, 10H), 1.37 (t, 3H).

LCMS m/z=871.3 [M+1]⁺

### Example 122

### 3-chloro-5-(2-(4-((2-(7-(4-(1-(2,6-dioxopiperidin-3-yl)-3-methyl-2-oxo-2,3-dihydro-1H-benzo[d]imidazol-5-yl)piperidin-1-yl)-2-azaspiro[3.5]nonan-2-yl)pyrimidin-4-yl)methoxy)phenyl)propan-2-yl)benzonitrile (Compound 122) trifluoroacetate

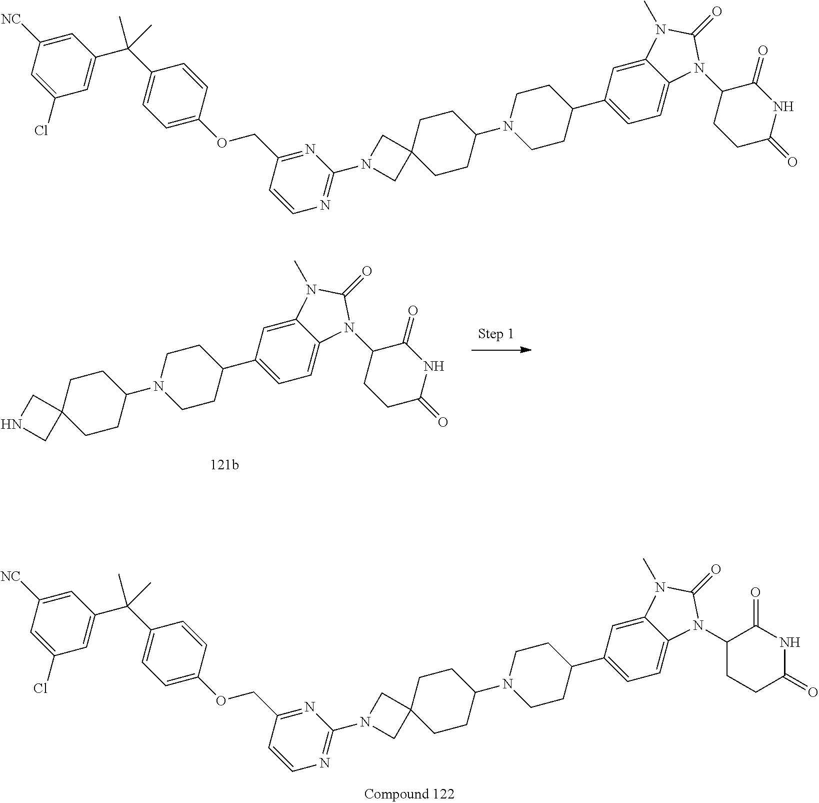 [SMILES: Cn1c(=O)n(C2CCC(=O)NC2=O)c2ccc(C3CCN(C4CCC5(CC4)CN(c4nccc(COc6ccc(C(C)(C)c7cc(Cl)cc(C#N)c7)cc6)n4)C5)CC3)cc21 ; Cn1c(=O)n(C2CCC(=O)NC2=O)c2ccc(C3CCN(C4CCC5(CC4)CNC5)CC3)cc21 ; Cn1c(=O)n(C2CCC(=O)NC2=O)c2ccc(C3CCN(C4CCC5(CC4)CN(c4nccc(COc6ccc(C(C)(C)c7cc(Cl)cc(C#N)c7)cc6)n4)C5)CC3)cc21]

The above crude 121b trifluoroacetate (2.00 g) was dissolved in 20 mL of DMF, and solid potassium bicarbonate (3.0 g, 30 mmol) was added. The mixture was stirred at room temperature for 0.5 h, and then 3-chloro-5-(2-(4-((2-chloropyrimidin-4-yl)methoxy)phenyl)propan-2-yl)benzonitrile (9d) (2.00 g, 4.88 mmol) was added. The resulting mixture was warmed to 80° C. and reacted for 4 h. The reaction liquid was cooled to room temperature, and water (20 mL) was added. The mixture was extracted with ethyl acetate (50 mL×3), and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 122 trifluoroacetate (0.65 g).

¹H NMR (400 MHz, DMSO-d₆) δ 11.08 (s, 1H), 8.35 (d, 1H), 7.88-7.82 (m, 1H), 7.69-7.62 (m, 1H), 7.56-7.50 (m, 1H), 7.22-7.14 (m, 2H), 7.12-6.88 (m, 5H), 6.75 (d, 1H), 5.43-5.27 (m, 1H), 4.97 (s, 2H), 3.82 (s, 2H), 3.73 (s, 2H), 3.50-3.42 (m, 2H), 3.35 (s, 3H), 3.32-3.07 (m, 3H), 3.00-2.84 (m, 2H), 2.79-2.58 (m, 2H), 2.20-1.87 (m, 9H), 1.73-1.49 (m, 10H).

LCMS m/z=827.3 [M+1]⁺

### Example 123

### 3-chloro-5-(2-(4-((2-(4-((1′-(1-(2,6-dioxopiperidin-3-yl)-6-fluoro-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-methoxybenzonitrile (Compound 123) trifluoroacetate

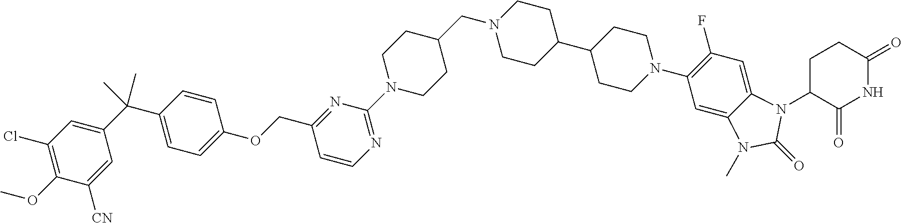 [SMILES: COc1c(Cl)cc(C(C)(C)c2ccc(OCc3ccnc(N4CCC(CN5CCC(C6CCN(c7cc8c(cc7F)n(C7CCC(=O)NC7=O)c(=O)n8C)CC6)CC5)CC4)n3)cc2)cc1C#N]

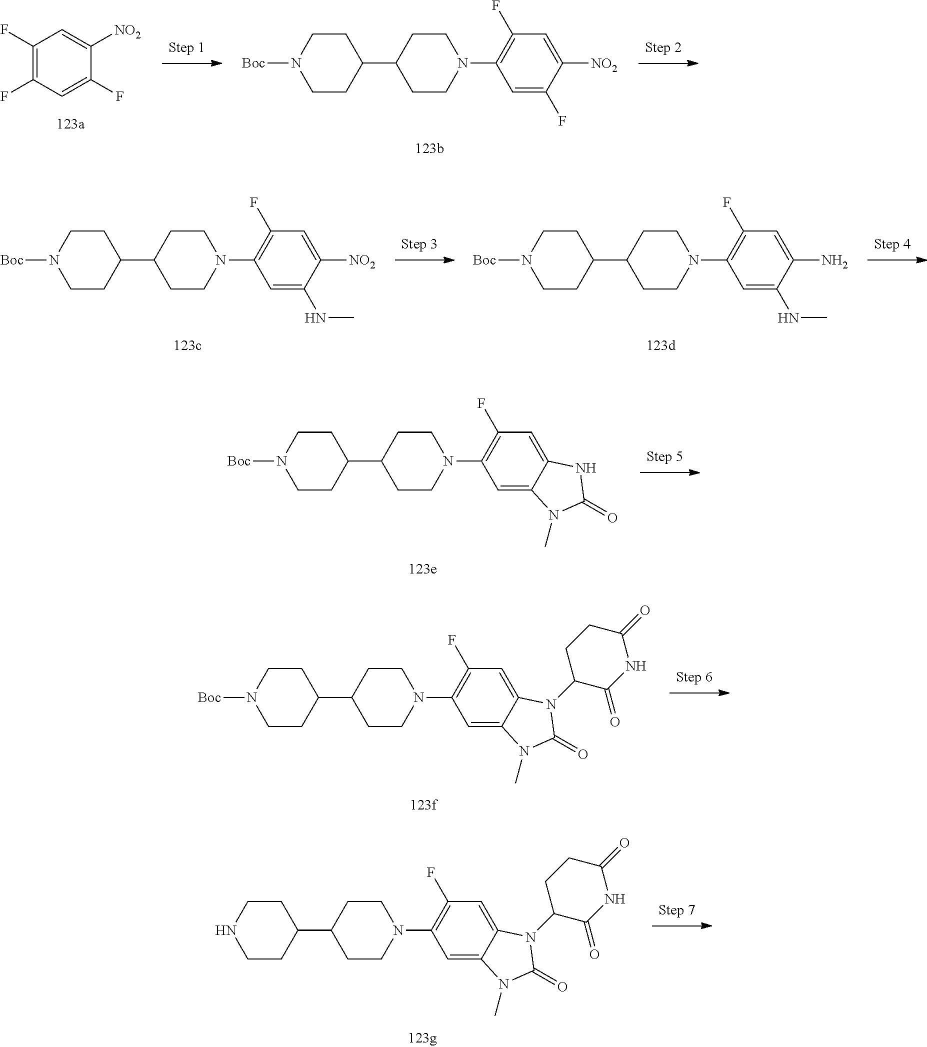

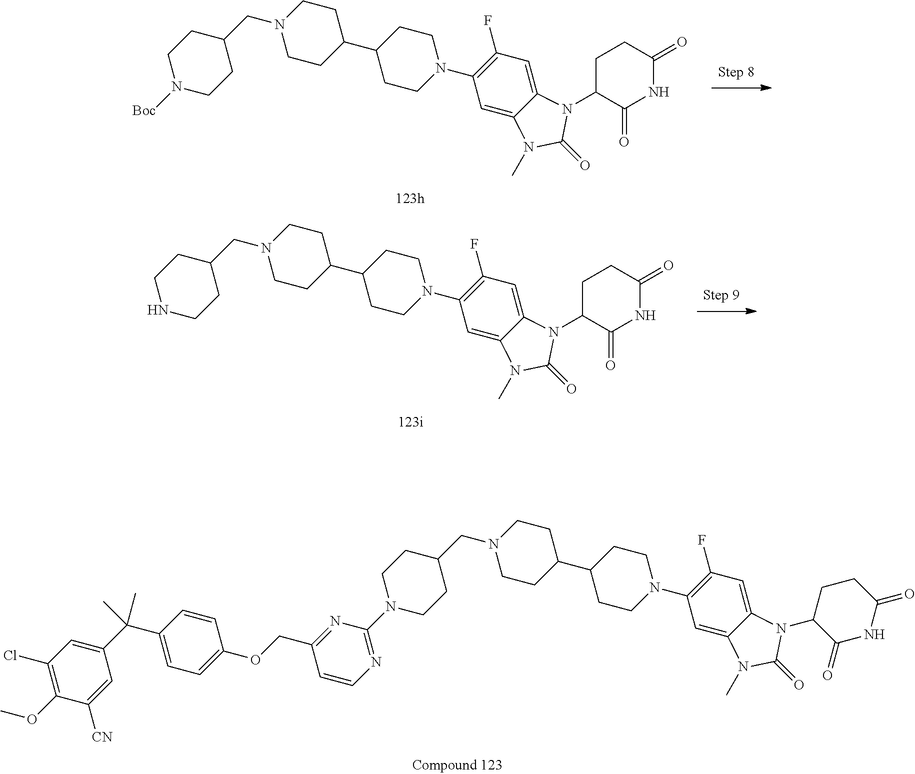 [SMILES: Cn1c(=O)n(C2CCC(=O)NC2=O)c2cc(F)c(N3CCC(C4CCN(CC5CCN(C(=O)OC(C)(C)C)CC5)CC4)CC3)cc21 ; Cn1c(=O)n(C2CCC(=O)NC2=O)c2cc(F)c(N3CCC(C4CCN(CC5CCNCC5)CC4)CC3)cc21 ; COc1c(Cl)cc(C(C)(C)c2ccc(OCc3ccnc(N4CCC(CN5CCC(C6CCN(c7cc8c(cc7F)n(C7CCC(=O)NC7=O)c(=O)n8C)CC6)CC5)CC4)n3)cc2)cc1C#N]

### Step 1: tert-butyl 1′-(2,5-difluoro-4-nitrophenyl)-[4,4′-bipiperidine]-1-carboxylate(123b)

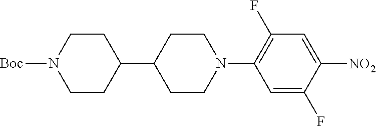 [SMILES: CC(C)(C)OC(=O)N1CCC(C2CCN(c3cc(F)c([N+](=O)[O-])cc3F)CC2)CC1]

Tert-butyl [4,4′-bipiperidine]-1-carboxylate (119a) (10 g, 37.26 mmol) and 1,2,4-trifluoro-5-nitrobenzene (123a) (6.92 g, 39.08 mmol) were dissolved in 80 mL of DMF, and solid potassium carbonate (5.66 g, 40.96 mmol) was added. The mixture was reacted at room temperature for 3 h. To the reaction liquid was added 500 mL of water, and the mixture was filtered by suction, to afford crude 123b (14.0 g).

LCMS m/z=426.3 [M+1]⁺

### Step 2: tert-butyl 1′-(2-fluoro-5-(methylamino)-4-nitrophenyl)-[4,4′-bipiperidine]-1-carboxylate (123c)

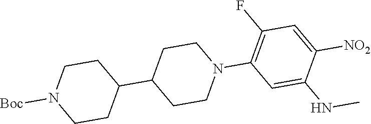 [SMILES: CNc1cc(N2CCC(C3CCN(C(=O)OC(C)(C)C)CC3)CC2)c(F)cc1[N+](=O)[O-]]

The above crude 123b (13 g) and methanamine hydrochloride (4.96 g, 73.46 mmol) were dissolved in 80 mL of acetonitrile, and solid potassium carbonate (10.25 g, 74.17 mmol) and potassium iodide (0.49 g, 2.95 mmol) were added. The mixture was placed in a sealed tube, warmed to 60° C. and reacted for 5 h. The reaction liquid was cooled to room temperature and filtered by suction. The filtrate was concentrated under reduced pressure, and the crude product was separated and purified by silica gel column chromatography (petroleum ether/ethyl acetate (v/v)=5:1-2:1), to afford 123c (12 g, two-step yield from compound 119a: 79%).

LCMS m/z=437.3 [M+1]⁺

### Step 3: tert-butyl 1′-(4-amino-2-fluoro-5-(methylamino)phenyl)-[4,4′-bipiperidine]-1-carboxylate (123d)

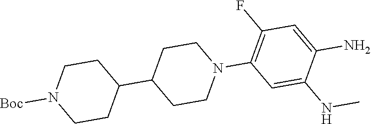 [SMILES: CNc1cc(N2CCC(C3CCN(C(=O)OC(C)(C)C)CC3)CC2)c(F)cc1N]

123c (12.0 g, 27.50 mmol) was dissolved in 150 mL of methanol, and 2.0 g of 10% Pd/C was added. The mixture was reacted at room temperature under hydrogen atmosphere for 15 h. The reaction liquid was filtered by suction. The filtrate was concentrated under reduced pressure, and the crude product was separated and purified by silica gel column chromatography (petroleum ether/ethyl acetate (v/v)=3:1-1:1), to afford 123d (8.5 g, yield: 76%).

### Step 4: tert-butyl 1′-(6-fluoro-3-methyl-2-oxo-2,3-dihydro-1H-benzo[d]imidazol-5-yl)-[4,4′-bipiperidine]-1-carboxylate (123e)

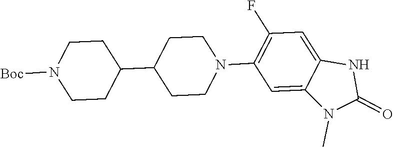 [SMILES: Cn1c(=O)[nH]c2cc(F)c(N3CCC(C4CCN(C(=O)OC(C)(C)C)CC4)CC3)cc21]

123d (8.5 g, 18.22 mmol) and CDI (5.0 g, 30.84 mmol) were dissolved in 80 mL of DCM, and triethylamine (4.2 g, 41.51 mmol) was added. The mixture was subjected to nitrogen replacement three times, warmed to 40° C. and reacted for 18 h. The reaction liquid was cooled to room temperature and filtered by suction. The filtrate was concentrated under reduced pressure, and the crude product was separated and purified by silica gel column chromatography (petroleum ether/ethyl acetate (v/v)=2:1-1:1), to afford 123e (8.2 g, yield: >99%).

LCMS m/z=433.2 [M+1]⁺

### Step 5: tert-butyl 1′-(1-(2,6-dioxopiperidin-3-yl)-6-fluoro-3-methyl-2-oxo-2,3-dihydro-1H-benzo[d]imidazol-5-yl)-[4,4′-bipiperidine]-1-carboxylate (123f)

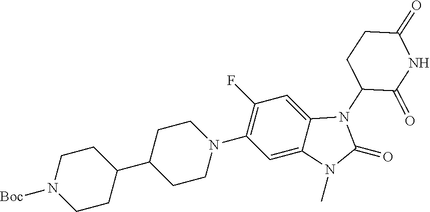 [SMILES: Cn1c(=O)n(C2CCC(=O)NC2=O)c2cc(F)c(N3CCC(C4CCN(C(=O)OC(C)(C)C)CC4)CC3)cc21]

123e (1.0 g, 2.31 mmol) was added to 30 mL of THF, and the mixture was subjected to nitrogen replacement three times and cooled to 0° C. 1 mol/L solution of LiHMDS in tetrahydrofuran (7 mL) was added, and the mixture was maintained at 0° C. and reacted for further 1 h. 3-bromopiperidine-2,6-dione (0.76 g, 3.96 mmol) was added, and the mixture was subjected to nitrogen replacement 3 times, warmed to 65° C. and reacted for 2 h. The reaction liquid was cooled to room temperature, and 40 mL of saturated aqueous ammonium chloride solution was added. The mixture was extracted with 100 mL of DCM, and the organic phase was washed with 50 mL of water, dried over anhydrous sodium sulfate, and concentrated under reduced pressure. The crude product was separated and purified by silica gel column chromatography (petroleum ether/ethyl acetate (v/v)=2:1-1:1), to afford 123f (0.16 g, yield: 13%).

LCMS m/z=544.3 [M+1]⁺

### Step 6: 3-(5-([4,4′-bipiperidin]-1-yl)-6-fluoro-3-methyl-2-oxo-2,3-dihydro-1H-benzo[d]imidazol-1-yl)piperidine-2,6-dione (123g) trifluoroacetate

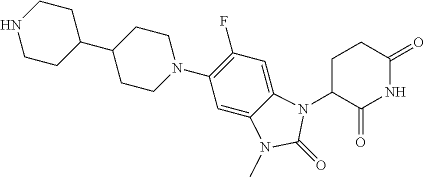 [SMILES: Cn1c(=O)n(C2CCC(=O)NC2=O)c2cc(F)c(N3CCC(C4CCNCC4)CC3)cc21]

123f (160 mg, 0.29 mmol) was dissolved in 10 mL of DCM, and 4 mL of trifluoroacetic acid was added. The mixture was reacted at room temperature for 2 h. The reaction liquid was concentrated under reduced pressure, to afford crude 123g trifluoroacetate (200 mg).

LCMS m/z=444.2 [M+1]⁺

### Step 7: tert-butyl 4-((1′-(1-(2,6-dioxopiperidin-3-yl)-6-fluoro-3-methyl-2-oxo-2,3-dihydro-1H-benzo[d]imidazol-5-yl)-[4,4′-bipiperidin]-1-yl)methyl)piperidine-1-carboxylate (123h)

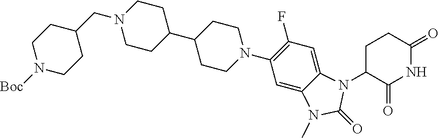 [SMILES: Cn1c(=O)n(C2CCC(=O)NC2=O)c2cc(F)c(N3CCC(C4CCN(CC5CCN(C(=O)OC(C)(C)C)CC5)CC4)CC3)cc21]

The above crude 123g trifluoroacetate (200 mg) was dissolved in 15 mL of THF, and solid sodium bicarbonate (75 mg, 0.89 mmol) was added. The mixture was stirred at room temperature for 20 min, and then tert-butyl 4-formylpiperidine-1-carboxylate (126 mg, 0.59 mmol) and 0.1 mL of acetic acid were successively added. The mixture was stirred at room temperature for 60 min, and then sodium triacetoxyborohydride (125 mg, 0.59 mmol) was added. The resulting mixture was reacted at room temperature for 16 h. To the reaction system was slowly added 10 mL of saturated sodium bicarbonate solution, and the mixture was extracted with 50 mL of DCM. The organic phase was washed with 20 mL of water, dried over anhydrous sodium sulfate, and concentrated under reduced pressure. The crude product was separated and purified by silica gel column chromatography (dichloromethane/methanol (v/v)=15:1), to afford 123h (160 mg, two-step yield from compound 123f: 86%).

LCMS m/z=641.4 [M+1]⁺

### Step 8: 3-(6-fluoro-3-methyl-2-oxo-5-(1′-(piperidin-4-ylmethyl)-[4,4′-bipiperidin]-1-yl)-2,3-dihydro-1H-benzo[d]imidazol-1-yl)piperidine-2,6-dione (123i) trifluoroacetate

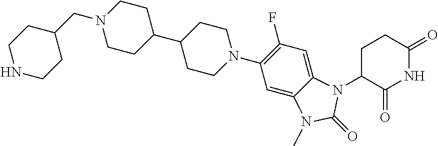 [SMILES: Cn1c(=O)n(C2CCC(=O)NC2=O)c2cc(F)c(N3CCC(C4CCN(CC5CCNCC5)CC4)CC3)cc21]

123h (160 mg, 0.25 mmol) was dissolved in 10 mL of DCM, and 4 mL of trifluoroacetic acid was added. The mixture was reacted at room temperature for 2 h. The reaction liquid was concentrated under reduced pressure, to afford crude 123i trifluoroacetate (200 mg).

LCMS m/z=541.3 [M+1]⁺

### Step 9: 3-chloro-5-(2-(4-((2-(4-((1′-(1-(2,6-dioxopiperidin-3-yl)-6-fluoro-3-methyl-2-oxo-2,3-dihydro-1H-benzo[d]imidazol-5-yl)-[4,4′-bipiperidin]-1-yl)methyl)piperidin-1-yl)pyrimidin-4-yl)methoxy)phenyl)propan-2-yl)-2-methoxybenzonitrile (Compound 123) trifluoroacetate

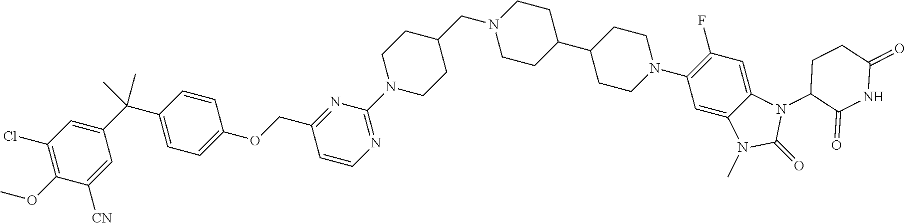 [SMILES: COc1c(Cl)cc(C(C)(C)c2ccc(OCc3ccnc(N4CCC(CN5CCC(C6CCN(c7cc8c(cc7F)n(C7CCC(=O)NC7=O)c(=O)n8C)CC6)CC5)CC4)n3)cc2)cc1C#N]

3-chloro-5-(2-(4-((2-chloropyrimidin-4-yl)methoxy)phenyl)propan-2-yl)-2-methoxybenzonitrile (92e) (145 mg, 0.34 mmol) was dissolved in 6 mL of DMF, and the above crude 123i trifluoroacetate (200 mg) and solid potassium bicarbonate (224 mg, 2.24 mmol) were added. The mixture was warmed to 60° C. and reacted for 3 h. The reaction liquid was cooled to room temperature, and 50 mL of water was added. The mixture was extracted with ethyl acetate (50 mL×3), and the organic phase was washed with water (50 mL×2), dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 123 trifluoroacetate (85 mg).

¹H NMR (400 MHz, CDCl₃) δ 8.90 (br.s, 1H), 8.33 (d, 1H), 7.54-7.27 (m, 3H), 7.16-7.06 (m, 2H), 6.95-6.73 (m, 4H), 5.30-5.13 (m, 1H), 4.99 (s, 2H), 4.87-4.65 (m, 2H), 4.03 (s, 3H), 3.89-3.50 (m, 4H), 3.50-3.19 (m, 5H), 3.19-2.49 (m, 9H), 2.33-2.10 (m, 2H), 2.09-1.74 (m, 9H), 1.63 (s, 6H), 1.56-1.21 (m, 5H).

LCMS m/z=932.4 [M+1]⁺

### Example 124

### 3-chloro-5-(2-(4-((2-(4-((4-(1-(2,6-dioxopiperidin-3-yl)-3-methyl-2-oxo-2,3-dihydro-1H-benzo[d]imidazol-4-yl)piperidin-1-yl)methyl)-4-fluoro-[1,4′-bipiperidin]-1′-yl)pyrimidin-4-yl)methoxy)phenyl)propan-2-yl)benzonitrile (Compound 124) trifluoroacetate

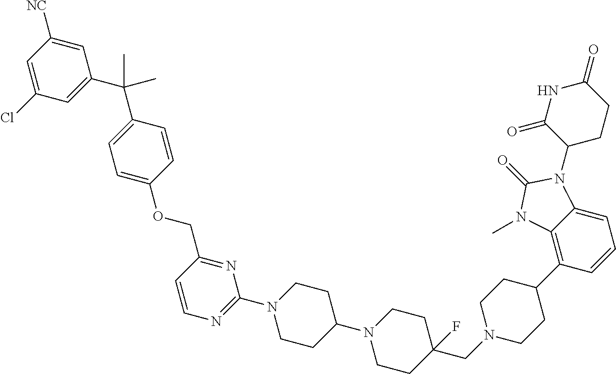 [SMILES: Cn1c(=O)n(C2CCC(=O)NC2=O)c2cccc(C3CCN(CC4(F)CCN(C5CCN(c6nccc(COc7ccc(C(C)(C)c8cc(Cl)cc(C#N)c8)cc7)n6)CC5)CC4)CC3)c21]

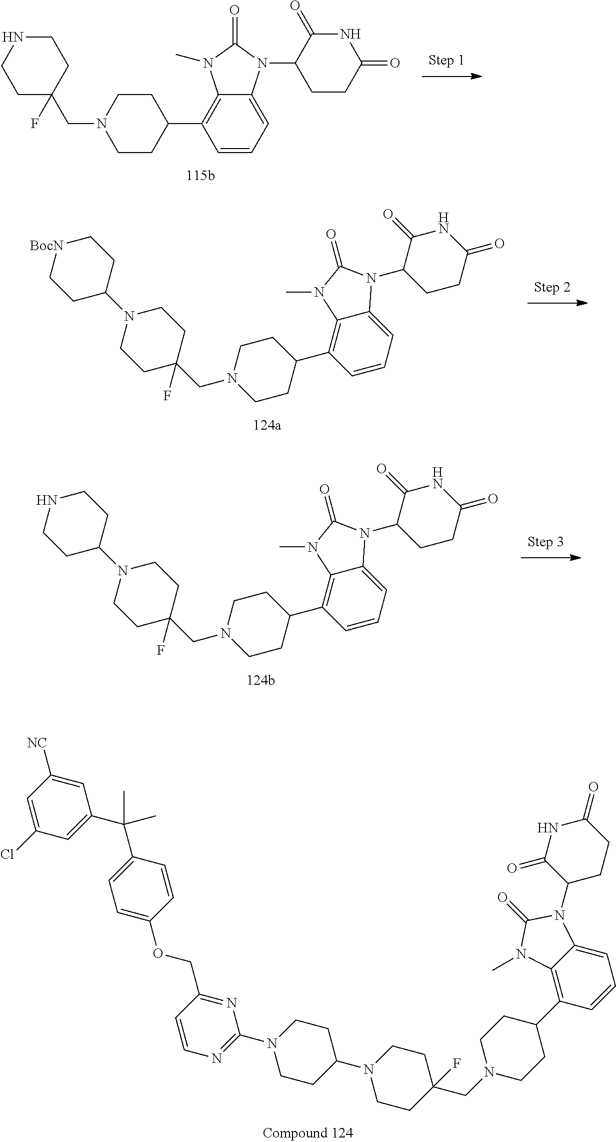 [SMILES: Cn1c(=O)n(C2CCC(=O)NC2=O)c2cccc(C3CCN(CC4(F)CCNCC4)CC3)c21 ; Cn1c(=O)n(C2CCC(=O)NC2=O)c2cccc(C3CCN(CC4(F)CCN(C5CCN(C(=O)OC(C)(C)C)CC5)CC4)CC3)c21 ; Cn1c(=O)n(C2CCC(=O)NC2=O)c2cccc(C3CCN(CC4(F)CCN(C5CCNCC5)CC4)CC3)c21 ; Cn1c(=O)n(C2CCC(=O)NC2=O)c2cccc(C3CCN(CC4(F)CCN(C5CCN(c6nccc(COc7ccc(C(C)(C)c8cc(Cl)cc(C#N)c8)cc7)n6)CC5)CC4)CC3)c21]

### Step 1: tert-butyl 4-((4-(1-(2,6-dioxopiperidin-3-yl)-3-methyl-2-oxo-2,3-dihydro-1H-benzo[d]imidazol-4-yl)piperidin-1-yl)methyl)-4-fluoro-[1,4′-bipiperidine]-1′-carboxylate (124a)

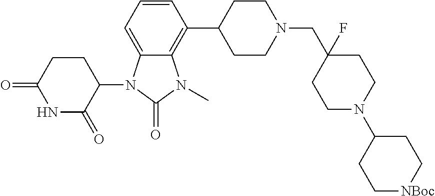 [SMILES: Cn1c(=O)n(C2CCC(=O)NC2=O)c2cccc(C3CCN(CC4(F)CCN(C5CCN(C(=O)OC(C)(C)C)CC5)CC4)CC3)c21]

The above crude 115b trifluoroacetate (318 mg) was dissolved in 5 mL of tetrahydrofuran, and dimethyl sulfoxide (0.5 mL) and solid sodium bicarbonate (0.151 g, 1.8 mmol) were added. The mixture was stirred at room temperature for 0.5 h, and then tert-butyl 4-oxopiperidine-1-carboxylate (0.48 g, 2.41 mmol) and glacial acetic acid (1.0 mL) were added. The mixture was warmed to 60° C. and reacted for 2 h, and the reaction liquid was cooled to room temperature. Sodium triacetoxyborohydride (0.50 g, 2.36 mmol) was added, and the resulting mixture was reacted at room temperature for 16 h. The reaction liquid was quenched with a saturated sodium bicarbonate solution (20 mL), and extracted with ethyl acetate (30 mL×3). The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 124a (76 mg, two-step yield from compound 115a: 18%).

### Step 2: 3-(4-(1-((4-fluoro-[1,4′-bipiperidin]-4-yl)methyl)piperidin-4-yl)-3-methyl-2-oxo-2,3-dihydro-1H-benzo[d]imidazol-1-yl)piperidine-2,6-dione (124b) trifluoroacetate

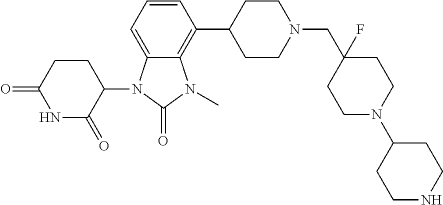 [SMILES: Cn1c(=O)n(C2CCC(=O)NC2=O)c2cccc(C3CCN(CC4(F)CCN(C5CCNCC5)CC4)CC3)c21]

124a (76 mg, 0.12 mmol) was dissolved in 2 mL of dichloromethane, and trifluoroacetic acid (1.53 g, 13.42 mmol) was added. The mixture was stirred at room temperature for 2 h. The reaction system was concentrated under reduced pressure, to afford crude 124b trifluoroacetate (80 mg).

### Step 3: 3-chloro-5-(2-(4-((2-(4-((4-(1-(2,6-dioxopiperidin-3-yl)-3-methyl-2-oxo-2,3-dihydro-1H-benzo[d]imidazol-4-yl)piperidin-1-yl)methyl)-4-fluoro-[1,4′-bipiperidin]-1′-yl)pyrimidin-4-yl)methoxy)phenyl)propan-2-yl)benzonitrile (Compound 124) trifluoroacetate

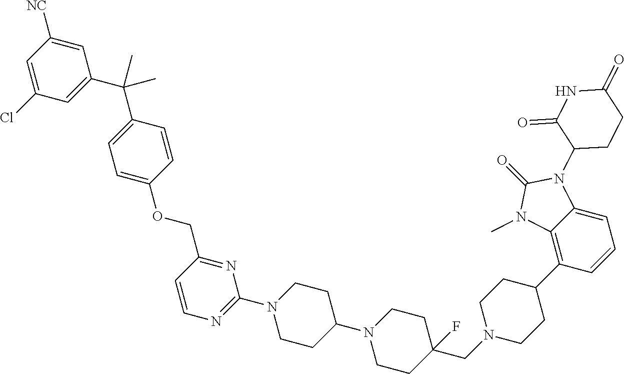 [SMILES: Cn1c(=O)n(C2CCC(=O)NC2=O)c2cccc(C3CCN(CC4(F)CCN(C5CCN(c6nccc(COc7ccc(C(C)(C)c8cc(Cl)cc(C#N)c8)cc7)n6)CC5)CC4)CC3)c21]

The above crude 124b trifluoroacetate (80 mg) was dissolved in 5 mL of N,N-dimethylformamide, and solid potassium bicarbonate (60 mg, 0.60 mmol) was added. The mixture was stirred at room temperature for 0.5 h, and then 3-chloro-5-(2-(4-((2-chloropyrimidin-4-yl)methoxy)phenyl)propan-2-yl)benzonitrile (9d) (80 mg, 0.194 mmol) was added. The resulting mixture was warmed to 80° C. and reacted for 4 h. The reaction liquid was cooled to room temperature, and 10 mL of water was added. The mixture was extracted with ethyl acetate (20 mL×3), and the organic phase was washed with 10 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 124 trifluoroacetate (35 mg, two-step yield from 124a: 32%).

¹H NMR (400 MHz, DMSO-d₆) δ 11.09 (s, 1H), 8.40 (d, 1H), 7.88-7.78 (m, 1H), 7.68-7.60 (m, 1H), 7.56-7.50 (m, 1H), 7.22-7.13 (m, 2H), 7.12-6.90 (m, 5H), 6.75 (d, 1H), 5.39 (dd, 1H), 5.00 (s, 2H), 4.90-4.79 (m, 2H), 3.76-3.45 (m, 11H), 3.44-3.25 (m, 2H), 3.24-3.04 (m, 2H), 3.00-2.81 (m, 3H), 2.80-2.55 (m, 2H), 2.47-2.30 (m, 2H), 2.28-1.90 (m, 9H), 1.74-1.50 (m, 8H).

LCMS m/z=902.4 [M+1]⁺

### Example 126

### 3-chloro-2-(2-chloroethoxy)-5-(2-(4-((2-(1′-((1-((1-(2,6-dioxopiperidin-3-yl)-3-methyl-2-oxo-2,3-dihydro-1H-benzo[d]imidazol-4-yl)methyl)piperidin-4-yl)methyl)-[4,4′-bipiperidin]-1-yl)pyrimidin-4-yl)methoxy)phenyl)propan-2-yl)benzonitrile (Compound 126) trifluoroacetate

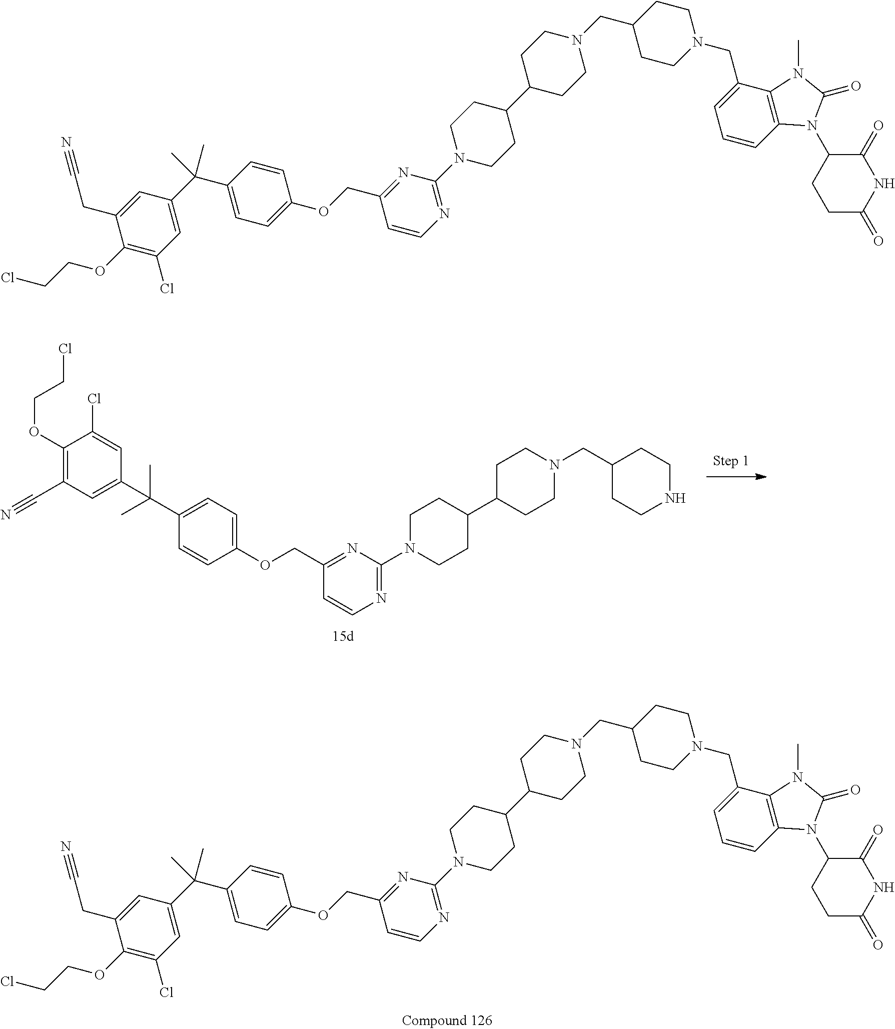 [SMILES: Cn1c(=O)n(C2CCC(=O)NC2=O)c2cccc(CN3CCC(CN4CCC(C5CCN(c6nccc(COc7ccc(C(C)(C)c8cc(Cl)c(OCCCl)c(CC#N)c8)cc7)n6)CC5)CC4)CC3)c21 ; CC(C)(c1ccc(OCc2ccnc(N3CCC(C4CCN(CC5CCNCC5)CC4)CC3)n2)cc1)c1cc(Cl)c(OCCCl)c(C#N)c1 ; Cn1c(=O)n(C2CCC(=O)NC2=O)c2cccc(CN3CCC(CN4CCC(C5CCN(c6nccc(COc7ccc(C(C)(C)c8cc(Cl)c(OCCCl)c(CC#N)c8)cc7)n6)CC5)CC4)CC3)c21]

The above crude 3-chloro-2-(2-chloroethoxy)-5-(2-(4-((2-(1′-(piperidin-4-ylmethyl)-[4,4′-bipiperidin]-1-yl)pyrimidin-4-yl)methoxy)phenyl)propan-2-yl)benzonitrile (15d) (52 mg) was dissolved in 5 mL of DMAc, and 1-(2,6-dioxopiperidin-3-yl)-3-methyl-2-oxo-2,3-dihydro-1H-benzo[d]imidazole-4-carbaldehyde (see WO 2020113233 for the synthetic method) (70 mg, 0.244 mmol) was added. The mixture was stirred at room temperature for 60 min, and then sodium triacetoxyborohydride (63 mg, 0.3 mmol) was added. The resulting mixture was stirred at room temperature for 16 h. To the reaction system was slowly added 30 mL of saturated sodium bicarbonate solution, and the mixture was extracted twice with 60 mL of ethyl acetate. The organic phase was washed with 50 mL of water, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min), lyophilization was performed to obtain compound 126 trifluoroacetate (8 mg).

¹H NMR (400 MHz, DMSO-d₆) δ 11.12 (s, 1H), 8.38-8.30 (m, 1H), 7.66-7.51 (m, 2H), 7.36-7.07 (m, 5H), 7.00-6.86 (m, 2H), 6.66 (d, 1H), 5.50-5.39 (m, 1H), 4.97 (s, 2H), 4.79-4.67 (m, 2H), 4.66-4.55 (m, 2H), 4.45-4.39 (m, 2H), 3.99-3.91 (m, 2H), 3.67-3.44 (m, 6H), 3.40-3.04 (m, 2H), 3.02-2.58 (m, 7H), 2.54-2.48 (m, 3H), 2.23-1.68 (m, 8H), 1.63 (s, 6H), 1.58-1.28 (m, 6H), 1.18-1.00 (m, 2H).

LCMS m/z=488.8 [M/2+1]⁺

### Example 127

### 3-chloro-5-(2-(4-(1-(2-(4-((1′-(2-(2,6-dioxopiperidin-3-yl)-6-fluoro-1,3-dioxoisoindolin-5-yl)-[4,4′-bipiperidin]-1-yl)methyl)piperidin-1-yl)pyrimidin-4-yl)ethoxy)phenyl)propan-2-yl)benzonitrile (Compound 127)

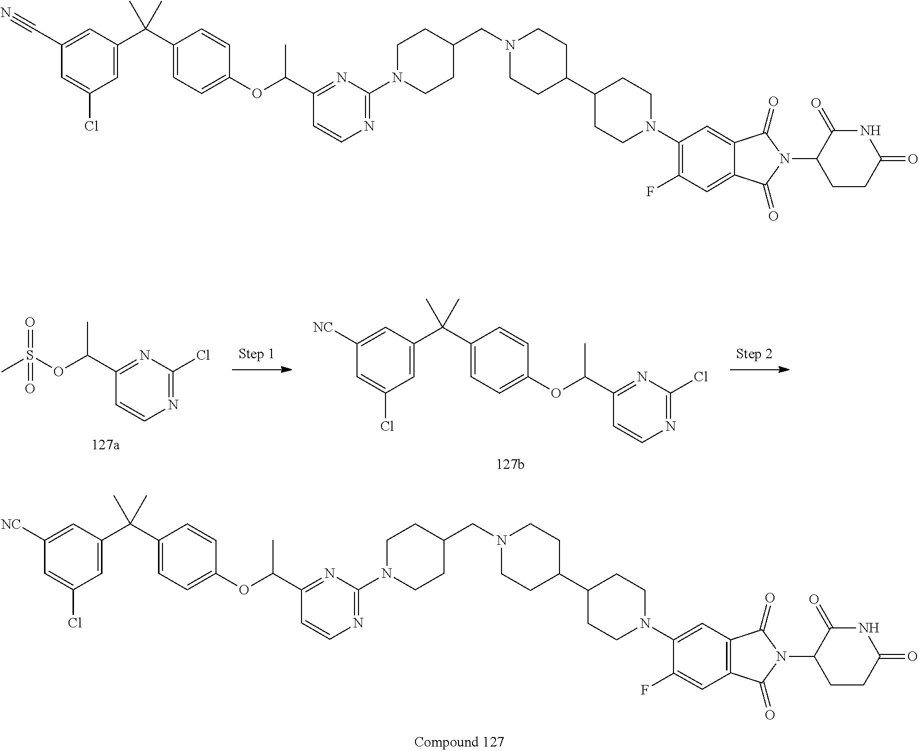 [SMILES: CC(Oc1ccc(C(C)(C)c2cc(Cl)cc(C#N)c2)cc1)c1ccnc(N2CCC(CN3CCC(C4CCN(c5cc6c(cc5F)C(=O)N(C5CCC(=O)NC5=O)C6=O)CC4)CC3)CC2)n1 ; CC(OS(C)(=O)=O)c1ccnc(Cl)n1 ; CC(Oc1ccc(C(C)(C)c2cc(Cl)cc(C#N)c2)cc1)c1ccnc(Cl)n1 ; CC(Oc1ccc(C(C)(C)c2cc(Cl)cc(C#N)c2)cc1)c1ccnc(N2CCC(CN3CCC(C4CCN(c5cc6c(cc5F)C(=O)N(C5CCC(=O)NC5=O)C6=O)CC4)CC3)CC2)n1]

### Step 1: 3-chloro-5-(2-(4-(1-(2-chloropyrimidin-4-yl)ethoxy)phenyl)propan-2-yl)benzonitrile (127b)

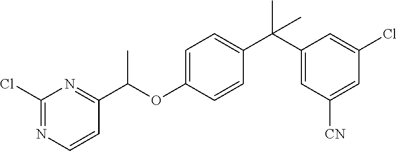 [SMILES: CC(Oc1ccc(C(C)(C)c2cc(Cl)cc(C#N)c2)cc1)c1ccnc(Cl)n1]

3-chloro-5-(2-(4-hydroxyphenyl)propan-2-yl)benzonitrile (9c) (0.50 g, 1.84 mmol) was dissolved in 15 mL of acetonitrile, and 1-(2-chloropyrimidin-4-yl)ethyl methanesulfonate (127a) (0.65 g, 2.75 mmol) (see WO 2020081999 for the synthetic method) and solid potassium carbonate (0.51 g, 3.69 mmol) were added. The mixture was warmed to 70° C. and reacted for 20 h. The reaction liquid was cooled to room temperature, and the reaction system was filtered and concentrated under reduced pressure. The crude product was separated and purified by silica gel column chromatography (ethyl acetate/petroleum ether (v/v)=0:1-1:5), to afford 127b (0.50 g, yield: 66%).

LCMS m/z=412.1 [M+1]⁺

### Step 2: 3-chloro-5-(2-(4-(1-(2-(4-((1′-(2-(2,6-dioxopiperidin-3-yl)-6-fluoro-1,3-dioxoisoindolin-5-yl)-[4,4′-bipiperidin]-1-yl)methyl)piperidin-1-yl)pyrimidin-4-yl)ethoxy)phenyl)propan-2-yl)benzonitrile (Compound 127)

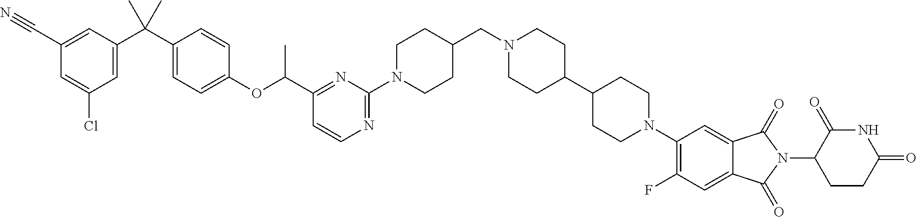 [SMILES: CC(Oc1ccc(C(C)(C)c2cc(Cl)cc(C#N)c2)cc1)c1ccnc(N2CCC(CN3CCC(C4CCN(c5cc6c(cc5F)C(=O)N(C5CCC(=O)NC5=O)C6=O)CC4)CC3)CC2)n1]

The above crude 2-(2,6-dioxopiperidin-3-yl)-5-fluoro-6-(1′-(piperidin-4-ylmethyl)-[4,4′-bipiperidin]-1-yl)isoindoline-1,3-dione (78e) trifluoroacetate (310 mg) was dissolved in 15 mL of DMF, and 127b (206 mg, 0.5 mmol) and solid potassium bicarbonate (500 mg, 5 mmol) were added. The mixture was warmed to 90° C. and reacted for 2 h. The mixture was cooled to room temperature, and 30 mL of water was added. The mixture was extracted twice with 30 mL of ethyl acetate, and the organic phase was washed with 50 mL of saturated sodium chloride solution, dried over anhydrous sodium sulfate, and concentrated under reduced pressure. The crude product was passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min). Then the preparative liquid was adjusted to pH 9 with a saturated sodium bicarbonate solution and extracted twice with 50 mL of ethyl acetate. The organic phase was washed twice with 50 mL of purified water, dried over anhydrous sodium sulfate, and concentrated under reduced pressure, to afford compound 127 (220 mg, yield from compound 127b: 48%).

¹H NMR (400 MHz, CDCl₃) δ 8.23 (d, 1H), 8.03 (br.s, 1H), 7.52-7.30 (m, 5H), 7.06-6.97 (m, 2H), 6.84-6.73 (m, 2H), 6.55 (d, 1H), 5.04 (q, 1H), 4.97-4.88 (m, 1H), 4.85-4.71 (m, 2H), 3.78-3.60 (m, 2H), 3.10-2.63 (m, 9H), 2.34-2.07 (m, 3H), 2.00-1.79 (m, 6H), 1.79-1.55 (m, 12H), 1.52-1.06 (m, 8H).

LCMS m/z=458.3 [M/2+1]⁺

### Example 128

### 3-chloro-5-(2-(4-((2-(9-(7-(2-(2,6-dioxopiperidin-3-yl)-1,3-dioxoisoindolin-5-yl)-7-azaspiro[3.5]nonan-2-yl)-3,9-diazaspiro[5.5]undecan-3-yl)pyrimidin-4-yl)methoxy)phenyl) propan-2-yl)benzonitrile (compound 128)

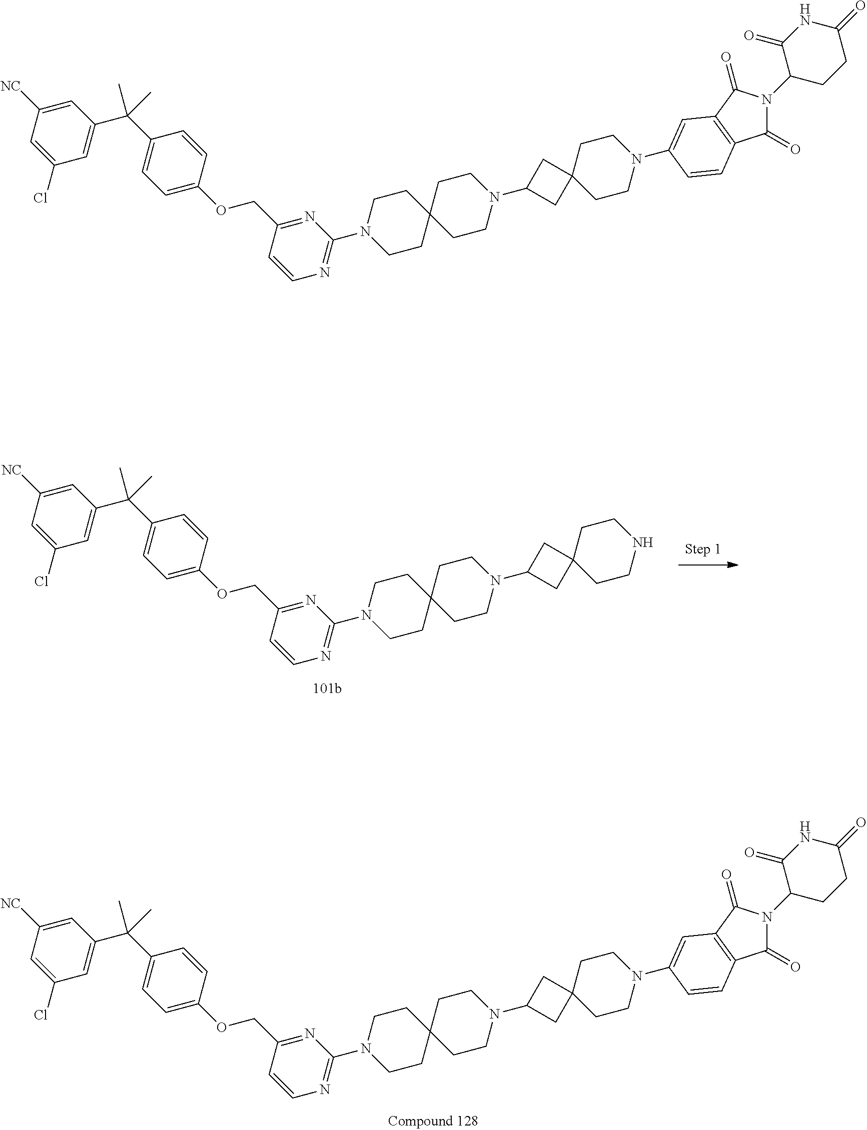 [SMILES: CC(C)(c1ccc(OCc2ccnc(N3CCC4(CC3)CCN(C3CC5(CCN(c6ccc7c(c6)C(=O)N(C6CCC(=O)NC6=O)C7=O)CC5)C3)CC4)n2)cc1)c1cc(Cl)cc(C#N)c1 ; CC(C)(c1ccc(OCc2ccnc(N3CCC4(CC3)CCN(C3CC5(CCNCC5)C3)CC4)n2)cc1)c1cc(Cl)cc(C#N)c1 ; CC(C)(c1ccc(OCc2ccnc(N3CCC4(CC3)CCN(C3CC5(CCN(c6ccc7c(c6)C(=O)N(C6CCC(=O)NC6=O)C7=O)CC5)C3)CC4)n2)cc1)c1cc(Cl)cc(C#N)c1]

To a reaction flask were successively added the above crude 3-(2-(4-((2-(9-(7-azaspiro[3.5]nonan-2-yl)-3,9-diazaspiro[5.5]undecan-3-yl)pyrimidin-4-yl)methoxy)phenyl) propan-2-yl)-5-chlorobenzonitrile (101b) (0.28 g), 2-(2,6-dioxopiperidin-3-yl)-5-fluoroisoindoline-1,3-dione (see WO 2017197056 for the synthetic method) (0.18 g, 0.65 mmol) and diisopropylethylamine (0.36 mL). 5 mL of dimethyl sulfoxide was added, and then the mixture was warmed to 80° C. and reacted for 4 h. The reaction liquid was cooled to room temperature, and 20 mL of water was added. The mixture was extracted with dichloromethane (20 mL×3), and the organic phase was washed with 20 mL of saturated sodium chloride solution, dried over anhydrous sodium sulfate, and concentrated under reduced pressure. The crude product was then separated and purified by silica gel column chromatography (dichloromethane/methanol (v/v)=10:1), to afford compound 128 (98 mg, two-step yield from compound 101a: 23%).

¹H NMR (400 MHz, DMSO-d₆) δ 11.05 (s, 1H), 8.35-8.29 (m, 1H), 7.87-7.81 (m, 1H), 7.68-7.60 (m, 2H), 7.55-7.49 (m, 1H), 7.34-7.11 (m, 4H), 6.98-6.90 (m, 2H), 6.63 (d, 1H), 5.06 (dd, 1H), 4.96 (s, 2H), 3.80-3.65 (m, 4H), 3.52-3.42 (m, 2H), 3.42-3.34 (m, 2H), 2.96-2.81 (m, 1H), 2.77-2.64 (m, 1H), 2.64-2.52 (m, 2H), 2.32-2.12 (m, 4H), 2.08-1.92 (m, 3H), 1.70-1.33 (m, 20H).

LCMS m/z=448.3 [M/2+1]⁺

### Example 129

### 3-chloro-5-(2-(4-((2-(9-((7-(2-(2,6-dioxopiperidin-3-yl)-1,3-dioxoisoindolin-5-yl)-7-azaspiro[3.5]nonan-2-yl)methyl)-3,9-diazaspiro[5.5]undecan-3-yl)pyrimidin-4-yl)methoxy)phenyl)propan-2-yl)benzonitrile (Compound 129)

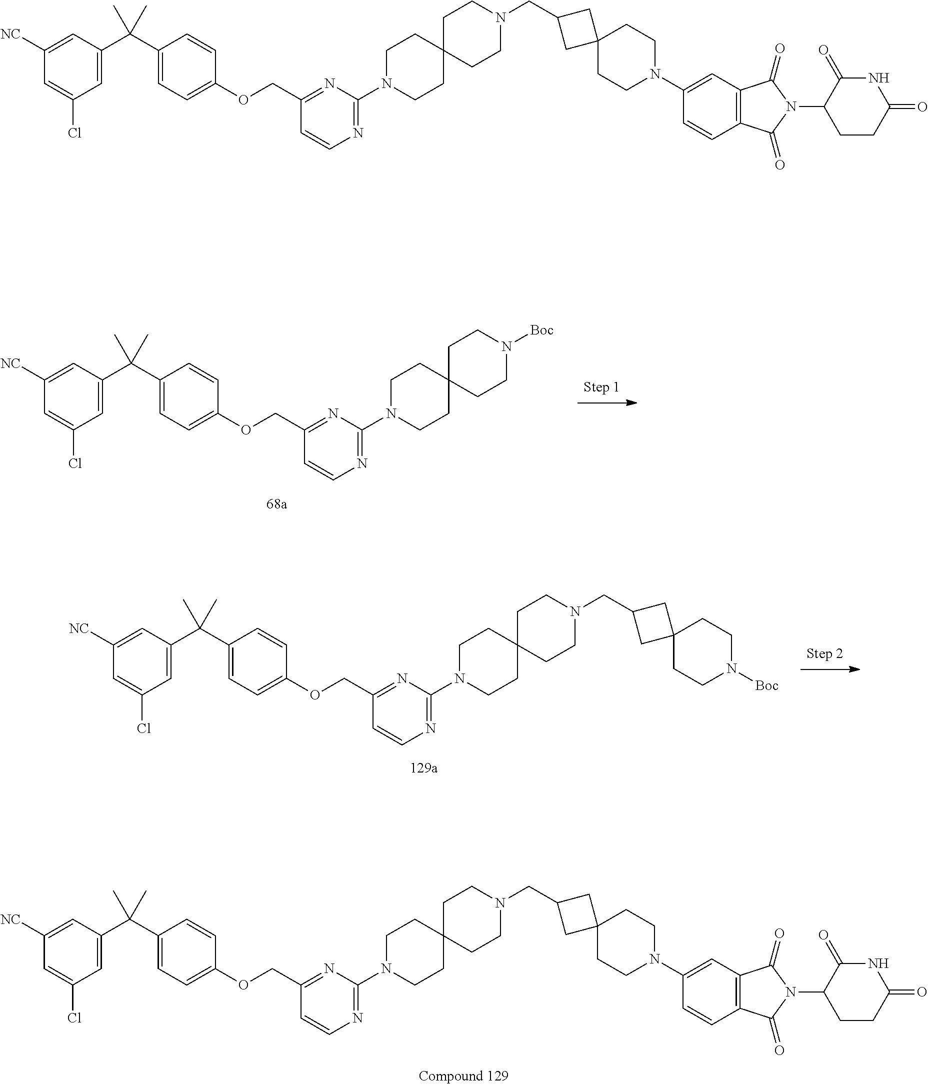 [SMILES: CC(C)(c1ccc(OCc2ccnc(N3CCC4(CCN(CC5CC6(CCN(c7ccc8c(c7)C(=O)N(C7CCC(=O)NC7=O)C8=O)CC6)C5)CC4)CC3)n2)cc1)c1cc(Cl)cc(C#N)c1 ; CC(C)(C)OC(=O)N1CCC2(CC1)CCN(c1nccc(COc3ccc(C(C)(C)c4cc(Cl)cc(C#N)c4)cc3)n1)CC2 ; CC(C)(C)OC(=O)N1CCC2(CC1)CC(CN1CCC3(CC1)CCN(c1nccc(COc4ccc(C(C)(C)c5cc(Cl)cc(C#N)c5)cc4)n1)CC3)C2 ; CC(C)(c1ccc(OCc2ccnc(N3CCC4(CCN(CC5CC6(CCN(c7ccc8c(c7)C(=O)N(C7CCC(=O)NC7=O)C8=O)CC6)C5)CC4)CC3)n2)cc1)c1cc(Cl)cc(C#N)c1]

### Step 1: tert-butyl 2-((9-(4-((4-(2-(3-chloro-5-cyanophenyl)propan-2-yl)phenoxy)methyl)pyrimidin-2-yl)-3,9-diazaspiro[5.5]undecan-3-yl)methyl)-7-azaspiro[3.5]nonane-7-carboxylate (129a)

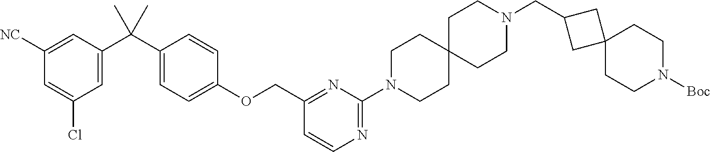 [SMILES: CC(C)(C)OC(=O)N1CCC2(CC1)CC(CN1CCC3(CC1)CCN(c1nccc(COc4ccc(C(C)(C)c5cc(Cl)cc(C#N)c5)cc4)n1)CC3)C2]

Tert-butyl 9-(4-((4-(2-(3-chloro-5-cyanophenyl)propan-2-yl)phenoxy)methyl)pyrimidin-2-yl)-3,9-diazaspiro[5.5]undecane-3-carboxylate (68a) (300 mg, 0.49 mmol) was dissolved in 7 mL of dichloromethane, and 3 mL of trifluoroacetic acid was added. The mixture was reacted at room temperature for 2 h. The reaction system was concentrated under reduced pressure and dissolved in 10 mL of tetrahydrofuran, and solid sodium bicarbonate (120 mg, 1.43 mmol) was added. The mixture was filtered by suction, the filtrate was concentrated under reduced pressure, and the residue was dissolved in 10 mL of tetrahydrofuran. Solid sodium bicarbonate (120 mg, 1.43 mmol) was added, and the mixture was reacted at room temperature for 0.5 h. Tert-butyl 2-formyl-7-azaspiro[3.5]nonane-7-carboxylate (see WO 2020264499 for the synthetic method) (250 mg, 0.99 mmol) was then added, and the mixture was reacted at room temperature for 1 h. Sodium triacetoxyborohydride (520 mg, 2.45 mmol) was then added, and the resulting mixture was reacted at room temperature for 12 h. To the reaction liquid was added 30 mL of dichloromethane, and the mixture was adjusted to pH 9.0 with a saturated aqueous sodium bicarbonate solution. The organic phase was separated, dried over anhydrous sodium sulfate, and concentrated under reduced pressure. The crude product was separated and purified by silica gel column chromatography (dichloromethane/methanol (v/v)=10:1), to afford 129a (430 mg, yield: >99%).

### Step 2: 3-chloro-5-(2-(4-((2-(9-((7-(2-(2,6-dioxopiperidin-3-yl)-1,3-dioxoisoindolin-5-yl)-7-azaspiro[3.5]nonan-2-yl)methyl)-3,9-diazaspiro[5.5]undecan-3-yl)pyrimidin-4-yl)methoxy)phenyl)propan-2-yl)benzonitrile (Compound 129)

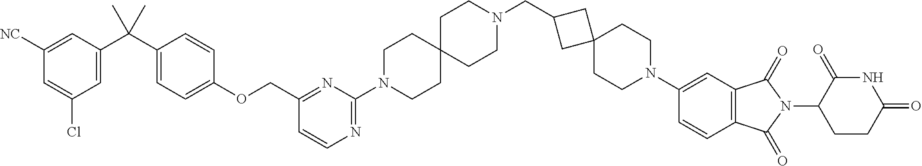 [SMILES: CC(C)(c1ccc(OCc2ccnc(N3CCC4(CCN(CC5CC6(CCN(c7ccc8c(c7)C(=O)N(C7CCC(=O)NC7=O)C8=O)CC6)C5)CC4)CC3)n2)cc1)c1cc(Cl)cc(C#N)c1]

129a (430 mg, 0.57 mmol) was dissolved in 8 mL of dichloromethane, and 4 mL of trifluoroacetic acid was added. The mixture was reacted at room temperature for 2 h. The reaction liquid was concentrated under reduced pressure and dissolved in 10 mL of dimethyl sulfoxide, and solid sodium bicarbonate (240 mg, 2.86 mmol) was added. The mixture was stirred at room temperature for 0.5 h, and then 2-(2,6-dioxopiperidin-3-yl)-5-fluoroisoindoline-1,3-dione (see WO 2017197056 for the synthetic method) (240 mg, 0.87 mmol) and diisopropylethylamine (0.47 mL) were added. The resulting mixture was warmed to 80° C. and reacted for 3 h. The reaction liquid was cooled to room temperature, and 20 mL of water was added. The mixture was extracted with ethyl acetate (30 mL×3), and the organic phase was washed with 20 mL of saturated sodium chloride solution, dried over anhydrous sodium sulfate, and concentrated under reduced pressure. The crude product was then separated and purified by silica gel column chromatography (dichloromethane/methanol (v/v)=10:1), to afford compound 129 (105 mg, yield: 20%).

¹H NMR (400 MHz, CDCl₃) δ 8.40 (br.s, 1H), 8.29 (d, 1H), 7.69-7.61 (m, 1H), 7.50-7.32 (m, 3H), 7.29-7.24 (m, 1H), 7.13-6.99 (m, 3H), 6.93-6.83 (m, 2H), 6.66 (d, 1H), 5.00-4.84 (m, 3H), 3.86-3.70 (m, 4H), 3.48-3.25 (m, 4H), 2.95-2.65 (m, 3H), 2.65-2.30 (m, 7H), 2.19-1.99 (m, 3H), 1.80-1.70 (m, 2H), 1.69-1.46 (m, 18H).

LCMS m/z=455.3 [M/2+1]⁺

### Example 130

### 3-chloro-5-(2-(4-((2-(3-((1′-(2-(2,6-dioxopiperidin-3-yl)-1,3-dioxoisoindolin-5-yl)-[4,4′-bipiperidin]-1-yl)methyl)pyrrolidin-1-yl)pyrimidin-4-yl)methoxy)phenyl)propan-2-yl)benzonitrile (Compound 130)

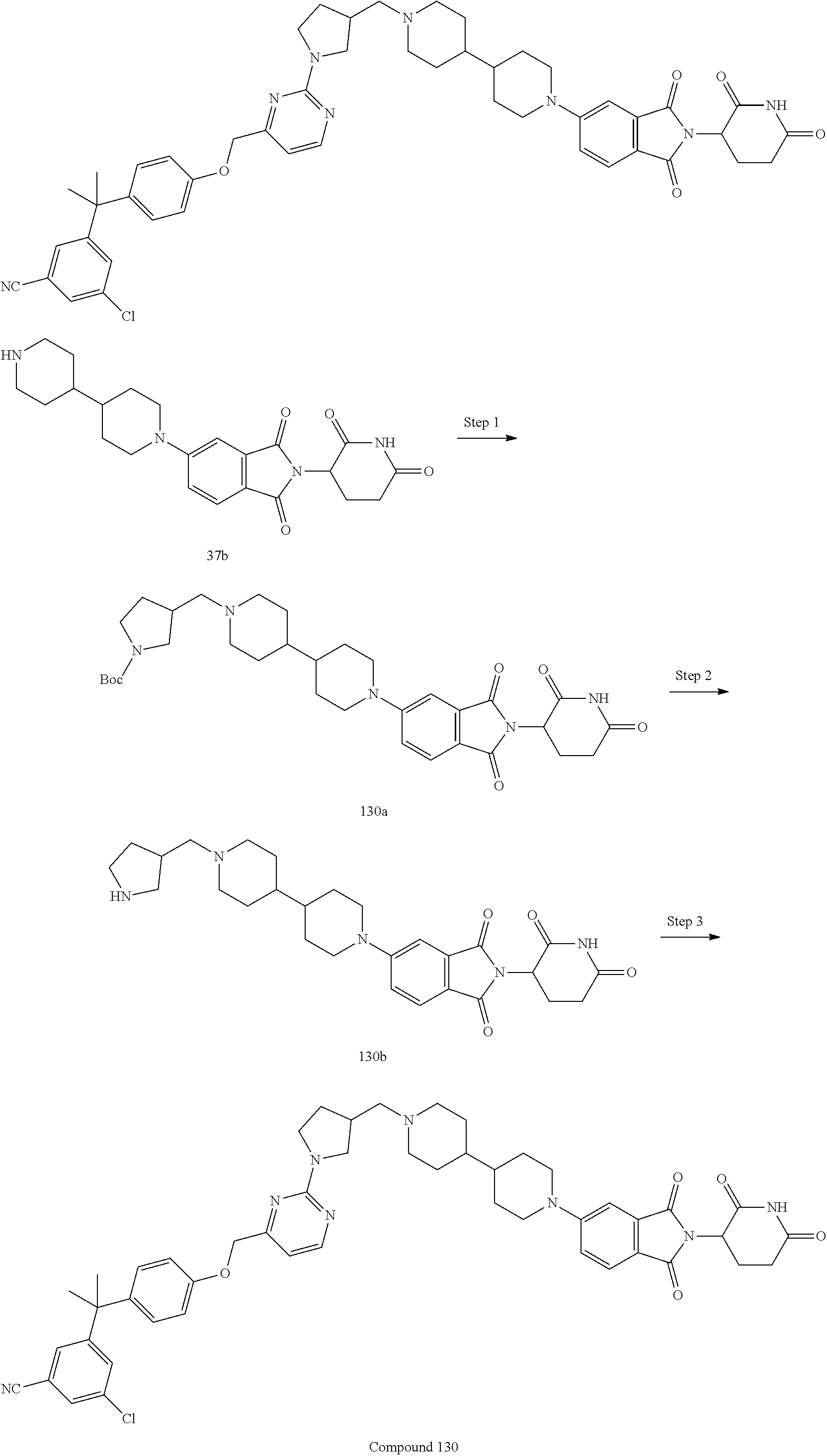

### Step 1: tert-butyl 3-((1′-(2-(2,6-dioxopiperidin-3-yl)-1,3-dioxoisoindolin-5-yl)-[4,4-bipiperidin]-1-yl)methyl)pyrrolidine-1-carboxylate (130a)

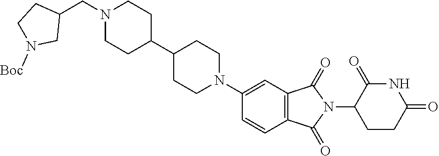 [SMILES: CC(C)(C)OC(=O)N1CCC(CN2CCC(C3CCN(c4ccc5c(c4)C(=O)N(C4CCC(=O)NC4=O)C5=O)CC3)CC2)C1]

The above crude 5-([4,4′-bipiperidin]-1-yl)-2-(2,6-dioxopiperidin-3-yl)isoindoline-1,3-dione (37b) (250 mg) was dissolved in 25 mL of 1,2-dichloroethane, and tert-butyl 3-formylpyrrolidine-1-carboxylate (240 mg, 1.20 mmol) was added. The mixture was stirred at room temperature for 1 h, and then sodium triacetoxyborohydride (380 mg, 1.79 mmol) was added. The resulting mixture was reacted at room temperature for 16 h. To the reaction liquid was slowly added 20 mL of saturated sodium bicarbonate solution, and the mixture was extracted twice with 60 mL of ethyl acetate. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 130a (230 mg, two-step yield from compound 37a: 48%).

LCMS m/z=608.3 [M+1]⁺

### Step 2: 2-(2,6-dioxopiperidin-3-yl)-5-(1′-(pyrrolidin-3-ylmethyl)-[4,4′-bipiperidin]-1-yl)isoindoline-1,3-dione (130b) trifluoroacetate

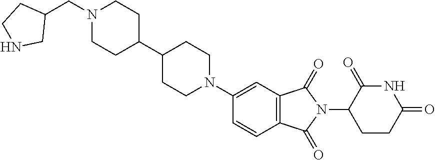 [SMILES: O=C1CCC(N2C(=O)c3ccc(N4CCC(C5CCN(CC6CCNC6)CC5)CC4)cc3C2=O)C(=O)N1]

Tert-butyl 3-((1′-(2-(2,6-dioxopiperidin-3-yl)-1,3-dioxoisoindolin-5-yl)-[4,4′-bipiperidin]-1-yl)methyl)pyrrolidine-1-carboxylate (130a) (230 mg, 0.38 mmol) was dissolved in 2 mL of DCM, and 1 mL of trifluoroacetic acid was added. The mixture was stirred at room temperature for 4 h. The reaction system was concentrated under reduced pressure, to afford crude 130b trifluoroacetate (230 mg).

LCMS m/z=508.3 [M+1]⁺

### Step 3: 3-chloro-5-(2-(4-((2-(3-((1′-(2-(2,6-dioxopiperidin-3-yl)-1,3-dioxoisoindolin-5-yl)-[4,4′-bipiperidin]-1-yl)methyl)pyrrolidin-1-yl)pyrimidin-4-yl)methoxy)phenyl)propan-2-yl)benzonitrile (Compound 130)

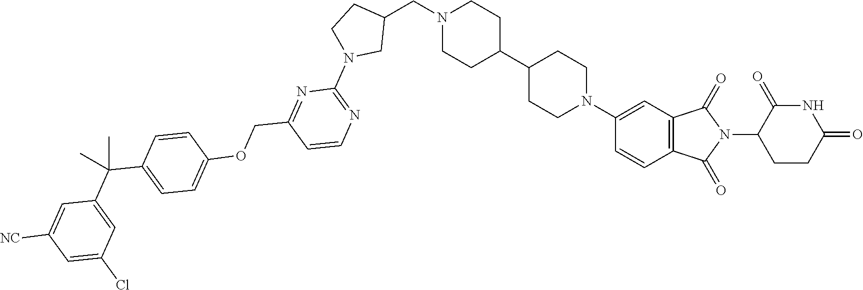 [SMILES: CC(C)(c1ccc(OCc2ccnc(N3CCC(CN4CCC(C5CCN(c6ccc7c(c6)C(=O)N(C6CCC(=O)NC6=O)C7=O)CC5)CC4)C3)n2)cc1)c1cc(Cl)cc(C#N)c1]

The above crude 130b trifluoroacetate (230 mg) was dissolved in 20 mL of DMF, and 3-chloro-5-(2-(4-((2-chloropyrimidin-4-yl)methoxy)phenyl)propan-2-yl)benzonitrile (9d) (150 mg, 0.38 mmol) and solid potassium bicarbonate (380 mg, 3.8 mmol) were successively added. The mixture was warmed to 90° C. and stirred for 4 h. The mixture was cooled to room temperature, and 30 mL of water was added. The mixture was extracted twice with 30 mL of ethyl acetate, and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min). The preparative liquid was adjusted to pH 9 with a saturated sodium bicarbonate solution and extracted twice with 50 mL of ethyl acetate. The organic phase was washed twice with 50 mL of purified water, dried over anhydrous sodium sulfate, and concentrated under reduced pressure, to afford compound 130 (150 mg, two-step yield from compound 130a: 45%).

¹H NMR (400 MHz, CDCl₃) δ 8.31 (d, 1H), 8.05 (s, 1H), 7.67 (d, 1H), 7.48-7.40 (m, 2H), 7.40-7.34 (m, 1H), 7.31-7.25 (m, 1H), 7.13-6.99 (m, 3H), 6.94-6.85 (m, 2H), 6.70 (d, 1H), 5.04-4.85 (m, 3H), 4.06-3.64 (m, 4H), 3.58-3.44 (m, 1H), 3.31-3.18 (m, 1H), 3.15-2.65 (m, 8H), 2.65-2.25 (m, 3H), 2.25-2.07 (m, 2H), 2.00-1.67 (m, 7H), 1.65 (s, 6H), 1.46-1.28 (m, 5H).

LCMS m/z=869.3 [M+1]⁺

### Example 131

### 3-chloro-5-(2-(4-((2-(1″-(2-(2,6-dioxopiperidin-3-yl)-1,3-dioxoisoindolin-5-yl)-[4,1′:4′,4″-terpiperidin]-1-yl)pyrimidin-4-yl)methoxy)phenyl)propan-2-yl)benzonitrile (Compound 131)

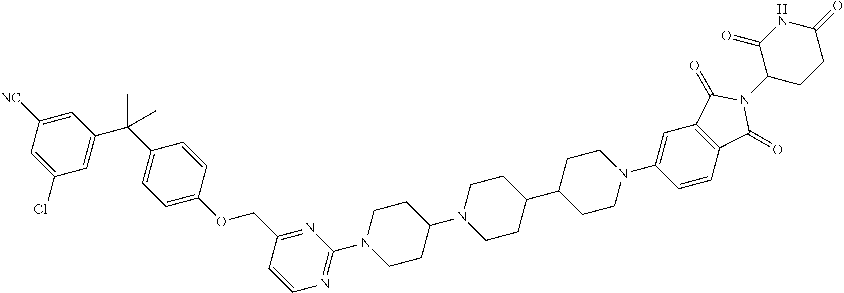 [SMILES: CC(C)(c1ccc(OCc2ccnc(N3CCC(N4CCC(C5CCN(c6ccc7c(c6)C(=O)N(C6CCC(=O)NC6=O)C7=O)CC5)CC4)CC3)n2)cc1)c1cc(Cl)cc(C#N)c1]

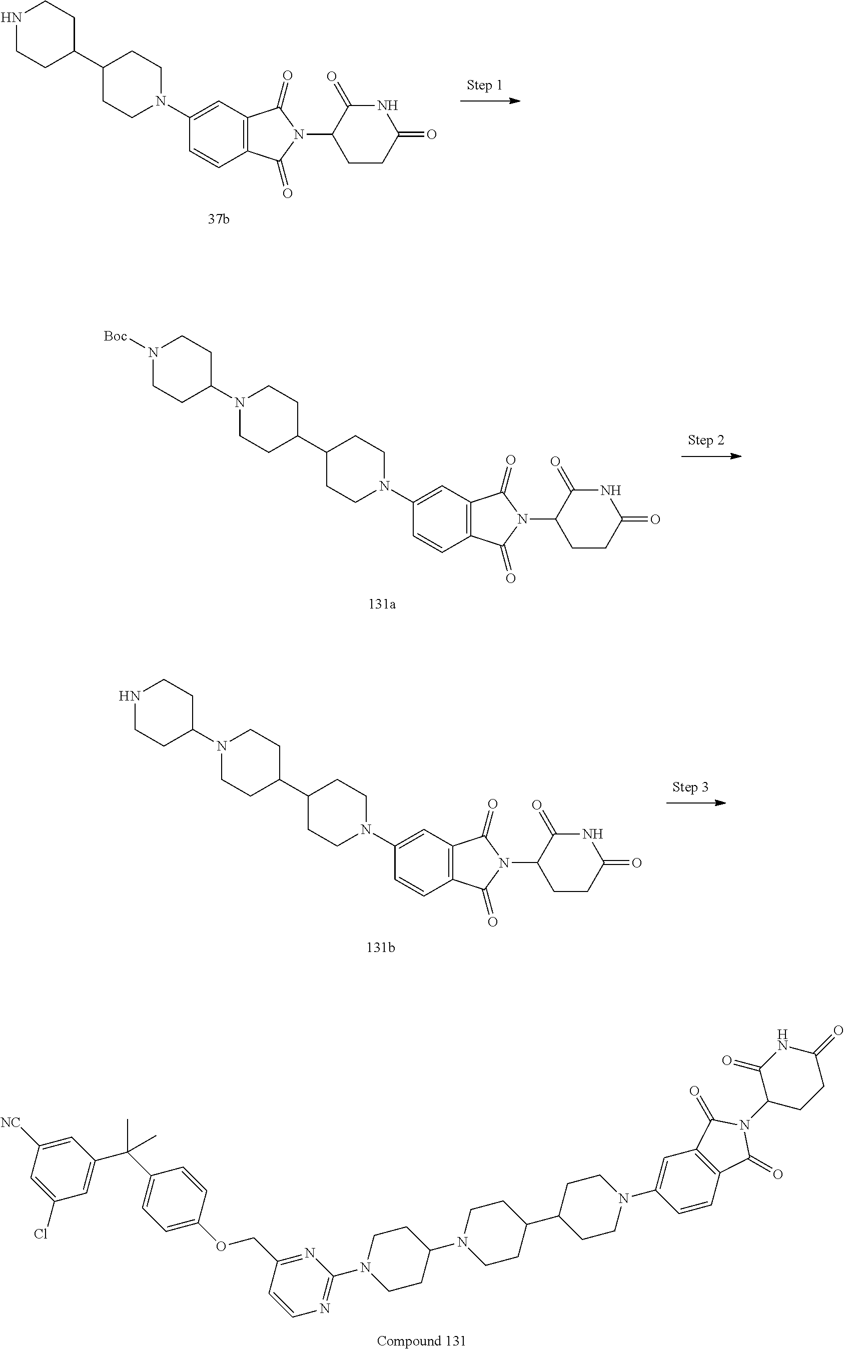 [SMILES: O=C1CCC(N2C(=O)c3ccc(N4CCC(C5CCNCC5)CC4)cc3C2=O)C(=O)N1 ; CC(C)(C)OC(=O)N1CCC(N2CCC(C3CCN(c4ccc5c(c4)C(=O)N(C4CCC(=O)NC4=O)C5=O)CC3)CC2)CC1 ; O=C1CCC(N2C(=O)c3ccc(N4CCC(C5CCN(C6CCNCC6)CC5)CC4)cc3C2=O)C(=O)N1 ; CC(C)(c1ccc(OCc2ccnc(N3CCC(N4CCC(C5CCN(c6ccc7c(c6)C(=O)N(C6CCC(=O)NC6=O)C7=O)CC5)CC4)CC3)n2)cc1)c1cc(Cl)cc(C#N)c1]

### Step 1: tert-butyl 1″-(2-(2,6-dioxopiperidin-3-yl)-1,3-dioxoisoindolin-5-yl)-[4,1′:4′,4″-terpiperidine]-1-carboxylate (131a)

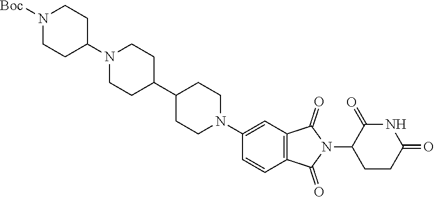 [SMILES: CC(C)(C)OC(=O)N1CCC(N2CCC(C3CCN(c4ccc5c(c4)C(=O)N(C4CCC(=O)NC4=O)C5=O)CC3)CC2)CC1]

The above crude 5-([4,4′-bipiperidin]-1-yl)-2-(2,6-dioxopiperidin-3-yl)isoindoline-1,3-dione (37b) (200 mg) was dissolved in 15 mL of 1,2-dichloroethane, and tert-butyl 4-oxopiperidine-1-carboxylate (200 mg, 1.01 mmol) was added. The mixture was stirred at room temperature for 1 h, and then sodium triacetoxyborohydride (300 mg, 1.42 mmol) was added. The resulting mixture was reacted at room temperature for 16 h. To the reaction liquid was slowly added 20 mL of saturated sodium bicarbonate solution, and the mixture was extracted twice with 60 mL of ethyl acetate. The organic phase was washed with 50 mL of saturated sodium chloride solution, dried over anhydrous sodium sulfate, and concentrated under reduced pressure. The crude product was separated and purified by silica gel column chromatography (dichloromethane/methanol (v/v)=10:1), to afford 131a (150 mg, two-step yield from compound 37a: 39%).

LCMS m/z=608.3 [M+1]⁺

### Step 2: 5-([4,1′:4′,4″-terpiperidin]-1″-yl)-2-(2,6-dioxopiperidin-3-yl)isoindoline-1,3-dione (131b) trifluoroacetate

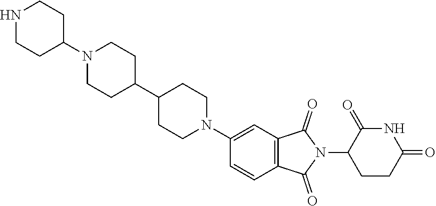 [SMILES: O=C1CCC(N2C(=O)c3ccc(N4CCC(C5CCN(C6CCNCC6)CC5)CC4)cc3C2=O)C(=O)N1]

Tert-butyl 1″-(2-(2,6-dioxopiperidin-3-yl)-1,3-dioxoisoindolin-5-yl)-[4,1′:4′,4″-terpiperidine]-1-carboxylate (131a) (150 mg, 0.25 mmol) was dissolved in 1 mL of DCM, and 0.5 mL of trifluoroacetic acid was added. The mixture was stirred at room temperature for 4 h. The reaction system was concentrated under reduced pressure, to afford crude 131b trifluoroacetate (150 mg).

LCMS m/z=508.0 [M+1]⁺

### Step 3: 3-chloro-5-(2-(4-((2-(1″-(2-(2,6-dioxopiperidin-3-yl)-1,3-dioxoisoindolin-5-yl)-[4,1′:4′,4″-terpiperidin]-1-yl)pyrimidin-4-yl)methoxy)phenyl)propan-2-yl)benzonitrile (Compound 131)

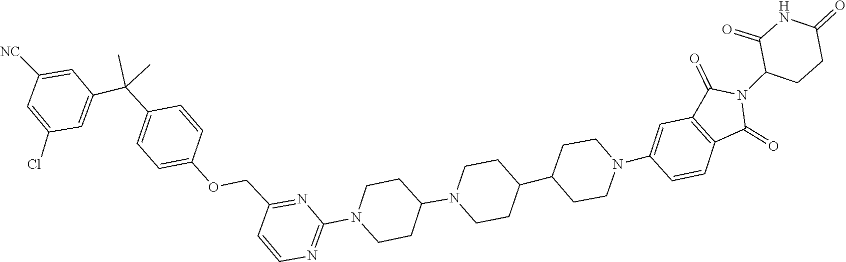 [SMILES: CC(C)(c1ccc(OCc2ccnc(N3CCC(N4CCC(C5CCN(c6ccc7c(c6)C(=O)N(C6CCC(=O)NC6=O)C7=O)CC5)CC4)CC3)n2)cc1)c1cc(Cl)cc(C#N)c1]

The above crude 131b trifluoroacetate (150 mg) was dissolved in 10 mL of DMF, and 3-chloro-5-(2-(4-((2-chloropyrimidin-4-yl)methoxy)phenyl)propan-2-yl)benzonitrile (9d) (120 mg, 0.30 mmol) and solid potassium bicarbonate (110 mg, 1.10 mmol) were successively added. The mixture was warmed to 90° C. and stirred for 4 h. The mixture was cooled to room temperature, and 30 mL of water was added. The mixture was extracted twice with 30 mL of ethyl acetate, and the organic phase was washed with 50 mL of saturated sodium chloride solution, dried over anhydrous sodium sulfate, and concentrated under reduced pressure. The crude product was then passed through Pre-HPLC (instrument and preparative column: using Glison GX-281 to prepare the liquid phase, preparative column model: Sunfire C18, 5 μm, inner diameter×length=30 mm×150 mm). Preparation method: the crude product was dissolved with methanol and dimethyl sulfoxide, and filtered with a 0.45 μm filter membrane, to prepare into a sample liquid. Mobile phase system: acetonitrile/water (containing 0.1% TFA). Gradient elution method: gradient elution with acetonitrile from 5% to 60% (elution time: 15 min). The preparative liquid was adjusted to pH 9 with a saturated sodium bicarbonate solution and extracted twice with 50 mL of ethyl acetate. The organic phase was washed twice with 50 mL of purified water, dried over anhydrous sodium sulfate, and concentrated under reduced pressure, to afford compound 131 (12 mg, two-step yield from compound 131a: 6%).

¹H NMR (400 MHz, CDCl₃) δ 8.30 (d, 1H), 8.03 (br.s, 1H), 7.66 (d, 1H), 7.47-7.40 (m, 2H), 7.40-7.33 (m, 1H), 7.29-7.25 (m, 1H), 7.14-6.99 (m, 3H), 6.93-6.83 (m, 2H), 6.69 (d, 1H), 5.02-4.78 (m, 5H), 4.06-3.90 (m, 2H), 3.30-2.60 (m, 10H), 2.40-1.69 (m, 11H), 1.65 (s, 6H), 1.63-1.47 (m, 4H), 1.40-1.19 (m, 2H).

LCMS m/z=869.8 [M+1]⁺

### Biological Test Examples

### 1. Experiment 1 on Inhibiting 22RV1 Cell Proliferation

Prostate cancer cells 22RV1 were purchased from ATCC, the cell medium was RPMI 1640+10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration, plated at 2000 cells/well and incubated overnight. On day 2, compounds at different concentrations were added, and the plate was placed in the incubator and incubated for further 5 days. After the incubation was completed, according to operation instructions for a CellTiter-Glo kit (Promega, G7573), 50 μL of CTG solution, which was already pre-melted and equilibrated to room temperature, was added to each well, and the mixtures were uniformly mixed for 2 min using a microplate shaker. The plate was placed at room temperature for 10 min, and then fluorescence signal values were measured using a microplate reader (PHERAstar FSX). The results were processed according to formula (1), the survival rate of the compound at different concentrations was calculated, and the IC₅₀ value of the compound with a survival rate of 50% was calculated using origin9.2 software with DoseResp function, wherein RLU_{compound} was the readout of the treated group, and RLU_{control} was the average value of the DMSO vehicle control group.

$$Growth \% = {RLU}_{c ompound} / RL {U}_{control} \times 100 \% \qquad formula ( 1 )$$

The results for IC₅₀ values on proliferation inhibition of 22RV1 cells were shown in Table 1.

TABLE 1
IC₅₀ values on proliferation inhibition of 22RV1 cells

| Serial | | |
|---|---|---|
| No. | Compound No. | IC₅₀ (μM) |
| 1 | Compound 1 | 0.835 |
| 2 | Compound 2 | 0.594 |
| 3 | Compound 3 | 1.237 |

Conclusion: the compounds of the present invention had a given inhibitory effect on proliferation of 22RV1 cells. For example, in the examples of the present invention, compounds 1-3 had IC₅₀ values of 0.5-1.5 μM on activity inhibition of 22RV1 cells. The specific values about activity were shown in Table 1.

### 2. Experiment 2 on Inhibiting 22RV1 Cell Proliferation

Prostate cancer cells 22RV1 were purchased from ATCC, the cell medium was RPMI 1640+10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration, plated at 2000 cells/well and incubated overnight. On day 2, compounds at different concentrations were added, and the plate was placed in the incubator and incubated for further 7 days. After the incubation was completed, according to operation instructions for a CellTiter-Glo kit (Promega, G7573), 50 μL of CTG solution, which was already pre-melted and equilibrated to room temperature, was added to each well, and the mixtures were uniformly mixed for 2 min using a microplate shaker. The plate was placed at room temperature for 10 min, and then fluorescence signal values were measured using a microplate reader (PHERAstar FSX). The results were processed according to formula (2), the survival rate of the compound at different concentrations was calculated, and the IC₅₀ value of the compound with a survival rate of 50% was calculated using origin9.2 software with DoseResp function, wherein RLU compound was the readout of the treated group, and RLU control was the average value of the DMSO vehicle control group.

$$Growth \% = RLU compound / RLU control \times 100 \% \qquad formula ( 2 )$$

The results for IC₅₀ values on proliferation inhibition of 22RV1 cells were shown in Table 2 and Table 3.

TABLE 2
IC50 values on proliferation inhibition of 22RV1 cells

| Serial | | IC₅₀ |
|---|---|---|
| No. | Compound No. | (μM) |
| 1 | Compound 5 | 1.3 |
| 2 | Compound 6 trifluoroacetate | 0.602 |
| 3 | Compound 7 | 0.959 |
| 4 | Compound 8 | 1.164 |
| 5 | Compound 9 trifluoroacetate | 0.582 |
| 6 | Compound 10 | 0.562 |
| 7 | Compound 11 | 0.356 |
| 8 | Compound 12 | 0.804 |
| 9 | Compound 13 | 0.721 |
| 10 | Compound 14 | 0.628 |
| 11 | Compound 16 | 0.802 |
| 12 | Compound 17 | 1.022 |
| 13 | Compound 18 | 0.452 |
| 14 | Compound 19 | 0.422 |
| | trifluoroacetate | |
| 15 | Compound 20 | 0.954 |
| 16 | Compound 21 | 0.507 |
| 17 | Compound 22 | 0.717 |
| | trifluoroacetate | |
| 18 | Compound 23 | 0.500 |
| 19 | Compound 24 | 0.569 |
| 20 | Compound 25 | 0.542 |
| 21 | Compound 26 | 0.548 |
| 22 | Compound 27 | 0.663 |
| 23 | Compound 28 | 0.634 |
| 24 | Compound 29 trifluoroacetate | 0.788 |
| 25 | Compound 30 trifluoroacetate | 0.834 |
| 26 | Compound 31 trifluoroacetate | 0.401 |
| 27 | Compound 32 | 0.325 |
| 28 | Compound 33 | 1.294 |
| 29 | Compound 34 trifluoroacetate | 0.461 |
| 30 | Compound 35 | 0.827 |
| 31 | Compound 37 trifluoroacetate | 0.507 |
| 32 | Compound 38 trifluoroacetate | 0.883 |
| 33 | Compound 39 trifluoroacetate | 0.936 |
| 34 | Compound 40 trifluoroacetate | 0.764 |
| 35 | Compound 41 trifluoroacetate | 0.633 |
| 36 | Compound 42 trifluoroacetate | 0.453 |
| 37 | Compound 43 trifluoroacetate | 0.094 |

TABLE 3
IC50 values on proliferation inhibition of 22RV1 cells

| Serial | | IC₅₀ |
|---|---|---|
| No. | Compound No. | (μM) |
| 1 | Compound 44 | 0.647 |
| | trifluoroacetate | |
| 2 | Compound 45 | 0.51 |
| 3 | Compound 46 | 0.461 |
| | trifluoroacetate | |
| 4 | Compound 47 | 0.724 |
| | trifluoroacetate | |
| 5 | Compound 48 | 0.608 |
| | trifluoroacetate | |
| 6 | Compound 49 | 0.494 |
| | trifluoroacetate | |
| 7 | Compound 50 | 0.398 |
| | trifluoroacetate | |
| 8 | Compound 51 | 0.825 |
| | trifluoroacetate | |
| 9 | Compound 52 | 0.956 |
| | trifluoroacetate | |
| 10 | Compound 53 | 0.623 |
| | trifluoroacetate | |
| 11 | Compound 54 | 0.414 |
| 12 | Compound 55 | 0.519 |
| | trifluoroacetate | |
| 13 | Compound 56 | 0.716 |
| | trifluoroacetate | |
| 14 | Compound 57 | 0.687 |
| | trifluoroacetate | |
| 15 | Compound 58 | 0.744 |
| | trifluoroacetate | |
| 16 | Compound 59 | 0.647 |
| | trifluoroacetate | |
| 17 | Compound 60 | 0.866 |
| | trifluoroacetate | |
| 18 | Compound 61 | 0.632 |
| | trifluoroacetate | |
| 19 | Compound 62 | 0.735 |
| | trifluoroacetate | |
| 20 | Compound 63 | 0.593 |
| | trifluoroacetate | |
| 21 | Compound 64 | 0.637 |
| | trifluoroacetate | |
| 22 | Compound 65 | 0.759 |
| | trifluoroacetate | |
| 23 | Compound 66 | 0.584 |
| | trifluoroacetate | |
| 24 | Compound 67 | 0.643 |
| | trifluoroacetate | |
| 25 | Compound 68 | 0.268 |
| 26 | Compound 69 | 0.371 |
| | trifluoroacetate | |
| 27 | Compound 70 | 0.612 |
| | trifluoroacetate | |
| 28 | Compound 71 | 0.811 |
| | trifluoroacetate | |
| 29 | Compound 72 | 0.464 |
| | trifluoroacetate | |
| 30 | Compound 73 | 0.097 |
| | trifluoroacetate | |
| 31 | Compound 74 | 0.474 |
| | trifluoroacetate | |
| 32 | Compound 75 | 0.275 |
| | trifluoroacetate | |
| 33 | Compound 76 | 0.475 |
| | trifluoroacetate | |
| 34 | Compound 77 | 0.192 |
| | trifluoroacetate | |
| 35 | Compound 78 | 0.246 |
| 36 | Compound 79 | 0.414 |
| 37 | Compound 81 | 0.484 |
| | trifluoroacetate | |
| 38 | Compound 82 | 0.413 |
| | trifluoroacetate | |
| 39 | Compound 83 | 0.58 |
| 40 | Compound 84 | 0.608 |
| 41 | Compound 85 | 0.316 |
| | trifluoroacetate | |
| 42 | Compound 86 | 0.278 |
| | trifluoroacetate | |
| 43 | Compound 87 | 0.303 |
| | trifluoroacetate | |
| 44 | Compound 88 | 0.899 |
| | trifluoroacetate | |
| 45 | Compound 90 | 0.627 |
| | trifluoroacetate | |
| 46 | Compound 91 | 0.427 |
| | trifluoroacetate | |
| 47 | Compound 92 | 0.9 |
| | trifluoroacetate | |
| 48 | Compound 93 | 0.554 |
| | trifluoroacetate | |
| 49 | Compound 94 | 0.678 |
| | trifluoroacetate | |
| 50 | Compound 97 | 0.421 |
| | trifluoroacetate | |
| 51 | Compound 98 | 0.621 |
| | trifluoroacetate | |
| 52 | Compound 99 | 0.427 |
| | trifluoroacetate | |
| 53 | Compound 100 | 0.577 |
| | trifluoroacetate | |
| 54 | Compound 101 | 0.699 |
| | trifluoroacetate | |
| 55 | Compound 102 | 0.641 |
| | trifluoroacetate | |
| 56 | Compound 103 | 0.823 |
| | trifluoroacetate | |
| 57 | Compound 104 | 0.664 |
| | trifluoroacetate | |
| 58 | Compound 105 | 0.575 |
| | trifluoroacetate | |
| 59 | Compound 106 | 0.55 |
| | trifluoroacetate | |
| 60 | Compound 107 | 0.526 |
| | trifluoroacetate | |
| 61 | Compound 108 | 0.251 |
| | trifluoroacetate | |
| 62 | Compound 109 | 0.33 |
| | trifluoroacetate | |
| 63 | Compound 110 | 0.446 |
| | trifluoroacetate | |
| 64 | Compound 111 | 0.29 |
| | trifluoroacetate | |
| 65 | Compound 112 | 0.463 |
| | trifluoroacetate | |
| 66 | Compound 113 | 0.419 |
| | trifluoroacetate | |
| 67 | Compound 114 | 0.665 |
| | trifluoroacetate | |
| 68 | Compound 115 | 0.955 |
| | trifluoroacetate | |
| 69 | Compound 116 | 0.821 |
| | trifluoroacetate | |
| 70 | Compound 118 | 1.107 |
| | trifluoroacetate | |
| 71 | Compound 119 | 0.78 |
| 72 | Compound 120 | 0.409 |
| 73 | Compound 121 | 0.842 |
| | trifluoroacetate | |
| 74 | Compound 122 | 1.008 |
| | trifluoroacetate | |
| 75 | Compound 123 | 0.527 |
| | trifluoroacetate | |
| 76 | Compound 124 | 0.576 |
| | trifluoroacetate | |
| 77 | Compound 126 | 0.611 |
| | trifluoroacetate | |
| 78 | Compound 127 | 0.549 |
| 79 | Compound 128 | 0.132 |
| 80 | Compound 129 | 0.569 |
| 81 | Compound 130 | 0.483 |
| 82 | Compound 131 | 0.507 |

Conclusion: the compounds of the present invention had a given inhibitory effect on proliferation of 22RV1 cells. For example, in the examples of the present invention, compounds 5, 7, 8, 10-14, 16-18, 20-21, 23-28, 32, 33, 35, 45, 54, 68, 78, 79, 83, 84, 119, 120 and 127-131, and compounds 6, 9, 19, 22, 29-31, 34, 37-43, 44, 46-53, 55-67, 69-77, 81-82, 85-94, 97-116 trifluoroacetate, compound 118 trifluoroacetate, compounds 121-124 trifluoroacetate and 126 trifluoroacetate had IC₅₀ values less than 5 μM on activity inhibition of 22RV1 cells. The specific values about activity were shown in Table 2 and Table 3.

### 3. Pharmacokinetic Test in Rats

Test objective: in this test, a single dose of test compounds was administered to SD rats intragastrically, and the concentrations of the test compounds in plasma of the rats were measured to evaluate pharmacokinetic characteristics of the test compounds in the rats.

Test animals: male SD rats, 200-250 g, 6-8 weeks old, 3 rats/compound, purchased from Chengdu Dossy Experimental Animals Co., Ltd.

Test method: on the day of the test, 3 SD rats were randomly grouped according to their body weights. The animals were fasted with water available for 12 to 14 hours one day before the administration of a test compound, and were fed 4 hours after the administration.

TABLE 4

| | | Administration information | | | | | | |
|---|---|---|---|---|---|---|---|---|
| | | | Administration | Administration | Administration | | | |
| | Number | Test | dosage* | concentration | volume | Collected | Mode of | |
| Group | Male | compound | (mg/kg) | (mg/mL) | (mL/kg) | sample | administration | Vehicle |
| G1 | 3 | Compound | 5 | 0.5 | 10 | Plasma | Oral | 5% DMSO + 5% |
| | | of the | | | | | (intragastrically) | Solutol + 30% |
| | | present | | | | | | PEG-400 + 60% |
| | | invention | | | | | | (20% SBE-β-CD) |

*Dosage is calculated on the basis of free base.

Sampling: before and after the administration, 0.1 mL of blood was taken from the orbits of the rats under isoflurane anesthesia, and placed in an EDTAK2 centrifuge tube. Centrifugation was carried out at 5000 rpm at 4C for 10 mm, and the plasma was collected.

Time points for plasma collection in G1 group: 0, 5 min, 15 min, 30 min, 1 h, 2 h, 4 h, 6 h, 8 h, and 24 h.

Before analysis and detection, all samples were stored at −80° C. The samples were analyzed quantitatively by LC-MS/MS.

TABLE 5
Pharmacokinetic parameters of compounds of
the present invention in plasma of rats

| | Mode of | |
|---|---|---|
| Test compound | administration* | AUC₀₋ₜ (ng/mL · h) |
| Compound 37 trifluoroacetate | i.g. (5 mg/kg) | 867 ± 66 |
| Compound 45 | i.g. (5 mg/kg) | 452 ± 137 |
| Compound 78 | i.g. (5 mg/kg) | 2352 ± 309 |
| Compound 88 trifluoroacetate | i.g. (5 mg/kg) | 740 ± 362 |
| Compound 94 trifluoroacetate | i.g. (5 mg/kg) | 1704 ± 335 |
| Compound 97 trifluoroacetate | i.g. (5 mg/kg) | 658 ± 133 |
| Compound 98 trifluoroacetate | i.g. (5 mg/kg) | 492 ± 266 |
| Compound 99 trifluoroacetate | i.g. (5 mg/kg) | 541 ± 80 |
| Compound 110 trifluoroacetate | i.g. (5 mg/kg) | 563 ± 350 |
| Compound 111 trifluoroacetate | i.g. (5 mg/kg) | 574 ± 59 |
| Compound 112 trifluoroacetate | i.g. (5 mg/kg) | 827 ± 211 |
| Compound 118 trifluoroacetate | i.g. (5 mg/kg) | 686 ± 336 |
| Compound 120 | i.g. (5 mg/kg) | 1431 ± 209 |
| Compound 123 trifluoroacetate | i.g. (5 mg/kg) | 1190 ± 724 |
| Compound 127 | i.g. (5 mg/kg) | 1857 ± 185 |
| Compound 128 | i.g. (5 mg/kg) | 604 ± 149 |
| Compound 129 | i.g. (5 mg/kg) | 812 ± 304 |
| Compound 130 | i.g. (5 mg/kg) | 665 ± 264 |
| Compound 131 | i.g. (5 mg/kg) | 446 ± 225 |

*Notes:
i.g. (intragastrical) administration of the compounds.

Conclusion: the compounds of the present invention had a given oral absorption in rats.

### 4. Degradation Experiment on Full-Length AR (AR-FL) and AR Splice Variants (AR-Vs) in 22RV1 Cells

Prostate cancer cells 22RV1 were purchased from ATCC, the cell medium was 1640+10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration and plated into a 6-well plate at 1 mL/well and 100000 cells/well. The next day, a 1% css-FBS phenol-free medium containing the compound to be tested was added, a 1% css-FBS phenol-free medium containing 0.2% DMSO was added to one well as DMSO vehicle control, and the 6-well plate was cultured in an incubator at 37° C. under 5% CO₂. After 24 h, the cells were digested with pancreatin and collected into a 1.5 mL centrifuge tube. 15 μL of RIPA lysate (containing 1×protease inhibitor cocktail) was added to each well, and the cells were lysed on ice for 15 min and then centrifuged at 12000 g and 4° C. for 10 min. The supernatant protein samples were collected, and protein quantification was performed by a BCA method. AR-FL and AR-Vs were detected using fully automatic protein expression quantitative analysis, which involved diluting the protein to be tested to 1 mg/mL. 4 μL of the diluted protein samples were added to 1 μL of 5×Master Mix (provided in the kit), and the prepared samples were denatured at 95° C. for 5 min and placed on ice for use. The primary antibodies, AR (CST, 5153S) and β-actin (CST, 3700), were diluted with Antibody Diluent II (provided in the kit) at a dilution ratio of 1:20 and 1:200, respectively. The secondary antibody was a mixed goat anti-mouse and goat anti-rabbit secondary antibody in a ratio of 1:1, and the color development solution was a mixed solution of Lumino-S and Peroxide in a ratio of 1:1. The prepared reagents were successively added to an assay plate according to instructions of the kit and detected on an instrument. Western blot band processing was performed using “Compass for SW”, a fully automatic protein expression quantitative analysis software, in which western blot bands were automatically simulated based on signal values. The degradation rate of AR-FL (3) or AR-Vs (4) relative to vehicle control at different compound concentrations was calculated according to formulas (3) and (4), wherein AR-FL_{compound} was the relative peak area of AR-FL in the treated group, and AR-FLₛₒₗᵥₑₙₜ was the relative peak area of AR-FL in the vehicle control group; AR-Vs_{compound} was the relative peak area of AR-Vs in the treated group, and AR-Vsₛₒₗᵥₑₙₜ was the relative peak area of AR-Vs in the vehicle control group.

$$Degradation rate of AR - FL = ( 1 - AR - {FL}_{c ompound} / AR - {FL}_{solvent} ) \times 100 \% \qquad formula ( 3 )$$

$$Degradation rate of AR - Vs = ( 1 - AR - {Vs}_{compound} / AR - {Vs}_{solvent} ) \times 100 \% \qquad formula ( 4 )$$

DC₅₀ calculation: the compound concentration DC₅₀ values at which the degradation rate of AR-FL or AR-Vs was 50% were calculated using OriginPro2015 software and analyzed using DoseResp function according to formula (3) or (4).

The results for DC₅₀ on degradation of AR-FL and AR-Vs by the compounds of the present invention were shown in Table 6.

TABLE 6
DC₅₀ on degradation of AR-FL and AR-Vs

| Serial | | AR-FL | AR-Vs |
|---|---|---|---|
| No. | Compound No. | DC₅₀ (μM) | DC₅₀ (μM) |
| 1 | Compound 97 | 1.184 | 1.067 |
| | trifluoroacetate | | |

The results for maximum degradation rate Dₘₐₓ% on AR-FL and AR-Vs by the compounds of the present invention were shown in Table 7.

TABLE 7
Maximum degradation rate Dₘₐₓ on degradation of AR-FL and AR-Vs

| Serial | | AR-FL | AR-Vs |
|---|---|---|---|
| No. | Compound No. | Dₘₐₓ % | Dₘₐₓ % |
| 1 | Compound 5 trifluoroacetate | 52.5 | — |
| 2 | Compound 6 trifluoroacetate | 57.6 | — |
| 3 | Compound 9 trifluoroacetate | 68.3 | 76.8 |
| 4 | Compound 10 | — | 64 |
| 5 | Compound 18 | 53 | 91 |
| 6 | Compound 88 trifluoroacetate | 64 | 73 |
| 7 | Compound 94 trifluoroacetate | 84 | 83 |
| 8 | Compound 110 trifluoroacetate | — | 56 |
| 10 | Compound 116 trifluoroacetate | 77 | 67 |
| 11 | Compound 126 trifluoroacetate | 98 | 95 |

Conclusion: the compounds of the present invention had a given degradation effect on AR-FL or AR-Vs in prostate cancer cells 22RV1.

### 5. Degradation Experiment on AR Splice Variant 7 (AR-V7) in 22RV1 Cells

Prostate cancer cells 22RV1 were purchased from ATCC, the cell medium was 1640+10% FBS, and the cells were cultured in an incubator at 37° C. under 5% CO₂. On day 1, the cells in an exponential phase were collected. With a 1% css-FBS phenol red-free medium, the cell suspension was adjusted to a corresponding concentration and plated into a 6-well plate at 1 mL/well and 300000 cells/well. The next day, a 1% css-FBS phenol-free medium containing the compound to be tested was added, a 1% css-FBS phenol-free medium containing 0.2% DMSO was added to one well as DMSO vehicle control, and the 6-well plate was cultured in an incubator at 37° C. under 5% CO₂. After 24 h, the cells were digested with pancreatin and collected into a 1.5 mL centrifuge tube. 15 μL of RIPA lysate (containing 1× protease inhibitor cocktail) was added to each well, and the cells were lysed on ice for 15 min and then centrifuged at 12000 g at 4° C. for 10 min. The supernatant protein samples were collected, and protein quantification was performed by a BCA method. AR-V7 was detected using fully automatic protein expression quantitative analysis, which involved diluting the protein to be tested to 2 mg/mL. 4 μL of the diluted protein samples were added to 1 μL of 5×Master Mix (provided in the kit), and the prepared samples were denatured at 95° C. for 5 min and placed on ice for use. The primary antibodies, AR V7 (CST, 19672S) and β-actin (CST, 3700), were diluted with Antibody Diluent II (provided in the kit) at a dilution ratio of 1:10 and 1:500, respectively. The secondary antibody was a mixed goat anti-mouse and goat anti-rabbit secondary antibody in a ratio of 1:1, and the color development solution was a mixed solution of Lumino-S and Peroxide in a ratio of 1:1. The prepared reagents were successively added to an assay plate according to instructions of the kit and detected on an instrument. Western blot band processing was performed using “Compass for SW”, a fully automatic protein expression quantitative analysis software, in which western blot bands were automatically simulated based on signal values. The degradation rate of AR-V7 (5) relative to vehicle control at different compound concentrations was calculated according to formula (5), wherein AR-V7_{compound} was the relative peak area of AR-V7 in the treated group, and AR-V7ₛₒₗᵥₑₙₜ was the relative peak area of AR-V7 in the vehicle control group.

$$AR - V 7 \% = ( 1 - AR - V {7}_{c ompound} / AR - V {7}_{solvent} ) \times 100 \% \qquad formula ( 5 )$$

DC₅₀ calculation: the compound concentration DC₅₀ values at which the degradation rate of AR-V7 was 50% were calculated using Graphpad software and analyzed using log(inhibitor) vs. response—Variable slope (four parameters) function according to formula (5).

The results for DC₅₀ on degradation of AR-V7 by the compounds of the present invention were shown in Table 8.

TABLE 8
DC₅₀ on degradation of AR-V7

| Serial | | AR-V7 DC50 |
|---|---|---|
| No. | Compound | (μM) |
| 1 | Compound 119 | 3.707 |

Conclusion: the compounds of the present invention had a given degradation effect on AR-V7 in prostate cancer cells 22RV1.

### 6. Pharmacokinetic Test in Mice

Test objective: a single dose of test compounds was administered to ICR mice intragastrically, and the concentrations of the test compounds in plasma of the mice were measured to evaluate pharmacokinetic characteristics of the test compounds in the mice.

Test animals: male ICR mice, about 22 g, 6-8 weeks old, 9 mice/compound, purchased from Chengdu Dossy Experimental Animals Co., Ltd.

Test method: on the day of the test, 9 ICR mice were randomly grouped according to their body weights. The animals were fasted with water available for 12 to 14 hours one day before the administration of a test compound, and were fed 4 hours after the administration. The administration was performed according to Table 9.

TABLE 9
Administration information

| | | Administration information | | | | | | |
|---|---|---|---|---|---|---|---|---|
| | | | Administration | Administration | Administration | | | |
| | Number | Test | dosage* | concentration | volume | Collected | Mode of | |
| Group | Male | compound | (mg/kg) | (mg/mL) | (mL/kg) | sample | administration | Vehicle |
| G1 | 9 | Compound | 5 | 0.5 | 10 | Plasma | Oral | 5% DMSO + 5% |
| | | of the | | | | | (intragastrically) | Solutol + 30% |
| | | present | | | | | | PEG-400 + 60% |
| | | invention | | | | | | (20% SBE-β-CD) |

*Dosage is calculated on the basis of free base.

Sampling: before and after the administration, 0.06 ml of blood was taken from the orbits of the mice under isoflurane anesthesia, and placed in an EDTAK2 centrifuge tube. Centrifugation was carried out at 5000 rpm at 4° C. for 10 min, and the plasma was collected.

Time points for blood collection in the G1 intragastric administration group: 0 min, 5 min, 15 min, 30 min, 1 h, 2 h, 4 h, 6 h, 8 h and 24 h. Before analysis and detection, all samples were stored at −80° C. The samples were analyzed quantitatively by LC-MS/MS.

Conclusion: the compounds of the present invention had a given oral absorption in mice.

### 7. Pharmacokinetic Test in Beagle Dogs

Test objective: a single dose of test compounds was administered to beagle dogs intravenously and intragastrically, and the concentrations of the test compounds in plasma of the beagle dogs were measured to evaluate pharmacokinetic characteristics of the test compounds in the beagle dogs.

Test animals: male beagle dogs, about 8-11 kg, 6 beagle dogs/compound, purchased from Beijing Marshall Biotechnology Co., Ltd.

Test method: on the day of the test, 6 beagle dogs were randomly grouped according to their body weights. The animals were fasted with water available for 12 to 14 hours one day before the administration of a test compound, and were fed 4 hours after the administration. The administration was performed according to Table 10.

TABLE 10
Administration information

| | | Administration information | | | | | | |
|---|---|---|---|---|---|---|---|---|
| | | | Administration | Administration | Administration | | | |
| | Number | Test | dosage* | concentration | volume | Collected | Mode of | |
| Group | Male | compound | (mg/kg) | (mg/mL) | (mL/kg) | sample | administration | Vehicle |
| G1 | 3 | Compound | 1 | 1 | 1 | Plasma | Intravenously | 5% DMA + 5% |
| | | of the | | | | | | Solutol + 90% Saline |
| G2 | 3 | present | 10 | 2 | 5 | | Oral | 5% DMSO + 5% |
| | | invention | | | | | (intragastrically) | Solutol + 30% |
| | | | | | | | | PEG-400 + 60% |
| | | | | | | | | (20% SBE-β-CD) |

*Dosage is calculated on the basis of free base.

Sampling: before and after the administration, 1 ml of blood was taken from the limb veins, and placed in an EDTAK2 centrifuge tube. Centrifugation was carried out at 5000 rpm at 4° C. for 10 min, and the plasma was collected.

Time points for blood collection: 0 min, 5 min, 15 min, 30 min, 1 h, 2 h, 4 h, 6 h, 8 h, 10 h, 12 h and 24 h. Before analysis and detection, all samples were stored at −80° C. The samples were analyzed quantitatively by LC-MS/MS.

Conclusion: the compounds of the present invention had a given oral absorption in beagle dogs. Compound 97 had an oral bioavailability higher than 15%.

### 8. Pharmacokinetic Test in Cynomolgus Monkeys

Test objective: a single dose of test compounds was administered to cynomolgus monkeys intragastrically, and the concentrations of the test compounds in plasma of the cynomolgus monkeys were measured to evaluate pharmacokinetic characteristics of the test compounds in the cynomolgus monkeys.

Test animals: male cynomolgus monkeys, about 3-5 kg, 3 cynomolgus monkeys/compound, purchased from Suzhou Xishan Zhongke Laboratory Animal Co., Ltd.

Test method: on the day of the test, 3 cynomolgus monkeys were randomly grouped according to their body weights. The animals were fasted with water available for 12 to 14 hours one day before the administration of a test compound, and were fed 4 hours after the administration. The administration was performed according to Table 11.

TABLE 11
Administration information

| | | Administration information | | | | | | |
|---|---|---|---|---|---|---|---|---|
| | | | Administration | Administration | Administration | | | |
| | Number | Test | dosage* | concentration | volume | Collected | Mode of | |
| Group | Male | compound | (mg/kg) | (mg/mL) | (mL/kg) | sample | administration | Vehicle |
| G1 | 3 | Compound | 5 | 1 | 5 | Plasma | Oral | 5% DMSO + 5% |
| | | of the | | | | | (intragastrically) | Solutol + 30% |
| | | present | | | | | | PEG-400 + 60% |
| | | invention | | | | | | (20% SBE-β-CD) |

*Dosage is calculated on the basis of free base.

Sampling: before and after the administration, 1 ml of blood was taken from the limb veins, and placed in an EDTAK2 centrifuge tube. Centrifugation was carried out at 5000 rpm at 4° C. for 10 min, and the plasma was collected.

Time points for blood collection in the G1 intragastric administration group: 0 min, 5 min, 15 min, 30 min, 1 h, 2 h, 4 h, 6 h, 8 h, 10 h, 12 h and 24 h. Before analysis and detection, all samples were stored at −80° C. The samples were analyzed quantitatively by LC-MS/MS.

Conclusion: the compounds of the present invention had a given oral absorption in cynomolgus monkeys.

Various modifications of the present invention, in addition to those described herein, will be apparent to those skilled in the art from the foregoing description. Such modifications are also intended to fall within the scope of the appended claims. Each reference cited in the present application, including all patents, patent applications, journal articles, books, and any other publications, is incorporated herein by reference in its entirety.

1. A compound or a stereoisomer, deuterated compound, solvate, prodrug, metabolite, pharmaceutically acceptable salt or co-crystal thereof, wherein the compound is selected from a compound as represented by general formula (I),
B-L-K (I);
L is selected from a bond or —C₁₋₅₀ hydrocarbyl-, wherein 0 to 20 methylene units in the hydrocarbyl are optionally further replaced by -Ak- or -Cy-;
each -Ak- is independently selected from —(CH₂)_{q}—, —(CH₂)_{q}—O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}—, —C(═O)—, —C(═O)—(CH₂)_{q}—NR^{L}, —(C≡C)_{q}— —CH═CH—, —Si(R^{L})₂—, —Si(OH)(R^{L})—, —Si(OH)₂—, —P(═O)(OR^{L})—, —P(═O)(R^{L})—, —S(═O)—, —S(═O)₂— or a bond;
each q is independently selected from 0, 1, 2, 3, 4, 5 or 6;
each R^{L} is independently selected from H, C₁₋₆ alkyl, 3- to 7-membered heterocyclyl, 3- to 7-membered cycloalkyl, phenyl or 5- to 6-membered heteroaryl;
each -Cy- is independently selected from a bond, 4- to 8-membered mono-heterocyclic ring, 4- to 10-membered fused heterocyclic ring, 5- to 12-membered spiro-heterocyclic ring, 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 heteroatoms selected from O, S or N;
B is selected from 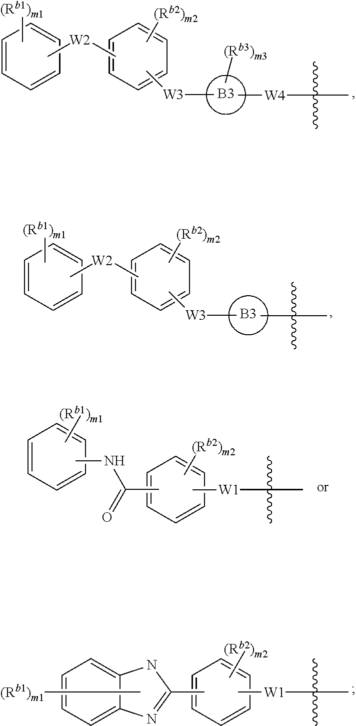
each W1 is independently selected from a bond, —O—, —S—, —NR^{w1}—, —NR^{w1}C(═O)—, —NR^{w1}S(═O)₂—, or —NR^{w1}S(═O)₂NR^{w1}—, wherein the left side thereof is directly linked to phenyl;
each W2 is independently selected from —NR^{w1}— or —(CR^{w2}R^{w3})ᵣ—, wherein the left side thereof is directly linked to phenyl;
each W3 is independently selected from —O(CR^{w2}R^{w3})ₜ—, —S(CR^{w2}R^{w3})ₜ—, or —NR^{w1}(CR^{w2}R^{w3})ₜ—, wherein the left side thereof is directly linked to phenyl;
W4 is selected from —O—, —S—, —NR^{w1}—, —NR^{w1}C(═O)—, —NR^{w1}S(═O)₂—, or —NR^{w1}S(═O)₂NR^{w1}—, wherein the left side thereof is directly linked to phenyl;
B3 is selected from 5- to 6-membered heteroaryl, wherein the heteroaryl contains 1 to 4 heteroatoms selected from O, S, or N;
each R^{w1} is independently selected from H, C₁₋₄ alkyl, or C₃₋₆ cycloalkyl;
each R^{w2} or R^{w3} is independently selected from H, F, Cl, Br, I, OH, CN, COOH, C₁₋₄ alkyl, C₁₋₄ alkenyl, C₁₋₄ alkynyl, C₁₋₄ alkoxy, —N(R^{b21})₂, —C(═O)N(R^{b21})₂ or C₃₋₆ cycloalkyl, wherein the alkyl, alkoxy, alkenyl, alkynyl or cycloalkyl is optionally further substituted with 0 to 4 substituents selected from H, F, Cl, Br, I, OH, ═O, NH₂, CN, COOH, C₁₋₄ alkyl, C₁₋₄ alkoxy, halogen-substituted C₁₋₄ alkyl, or cyano-substituted C₁₋₄ alkyl;
alternatively, R^{w2} and R^{w3} together with the carbon atoms to which they are attached form C₃₋₆ cycloalkyl or 3- to 8-membered heterocyclyl, wherein the cycloalkyl or heterocyclyl is optionally further substituted with 0 to 4 substituents selected from H, F, Cl, Br, I, OH, ═O, NH₂, CN, COOH, C₁₋₄ alkyl, C₁₋₄ alkoxy, halogen-substituted C₁₋₄ alkyl, or cyano-substituted C₁₋₄ alkyl, and the heterocyclyl contains 1 to 4 heteroatoms selected from O, S, or N;
each R^{b1} or R^{b2} is independently selected from H, F, Cl, Br, I, ═O, OH, CN, NO₂, COOH, C₁₋₄ alkyl, C₁₋₄ alkenyl, C₁₋₄ alkynyl, C₁₋₄ alkoxy, C₁₋₄ deuterated alkoxy, C₁₋₄ alkylthio, —(CH₂)ₙ—R^{b21}, —OR^{b21}, —N(R^{b21})₂, —C(═O)N(R^{b21})₂, —C(═O)OR^{b21}, —C(═O)R^{b22}, —NR^{b21}C(═O)R^{b22}, —NR^{b21}S(═O)₂R^{b22}, C₃₋₆ cycloalkyl, C₆₋₁₀ aryl, 5- to 10-membered heteroaryl or 4- to 10-membered heterocyclyl, wherein the alkyl, alkenyl, alkynyl, alkoxy, alkylthio, cycloalkyl, heterocyclyl, aryl or heteroaryl is optionally further substituted with 0 to 4 substituents selected from H, F, Cl, Br, I, OH, ═O, —N(R^{b21})₂, CN, COOH, C₁₋₄ alkyl, C₁₋₄ alkoxy, halogen-substituted C₁₋₄ alkyl, cyano-substituted C₁₋₄ alkyl, C₃₋₆ cycloalkyl, 5- to 10-membered heteroaryl or 4- to 10-membered heterocyclyl, and the heteroaryl or heterocyclyl contains 1 to 4 heteroatoms selected from O, S, or N;
each R^{b3} is independently selected from H, F, Cl, Br, I, ═O, OH, CN, COOH, C₁₋₄ alkyl, C₁₋₄ alkenyl, C₁₋₄ alkynyl, C₁₋₄ alkoxy, C₁₋₄ alkylthio, —(CH₂)ₙ—R^{b21}, —OR^{b21}, —N(R^{b21})₂, —C(═O)N(R^{b21})₂, —NR^{b21}C(═O)R^{b22}, —S(═O)₂N(R^{b21})₂, —NR^{b21}S(═O)₂R^{b22}, —C(═O)R^{b22}, or —S(═O)₂R^{b22}, wherein the alkyl, alkoxy, alkenyl, alkynyl or alkylthio is optionally further substituted with 0 to 4 substituents selected from H, F, Cl, Br, I, OH, ═O, —N(R^{b21})₂, CN, COOH, C₁₋₄ alkyl, C₁₋₄ alkoxy, halogen-substituted C₁₋₄ alkyl, or cyano-substituted C₁₋₄ alkyl;
each R^{b21} is independently selected from H, C₁₋₄ alkyl, C₃₋₆ cycloalkyl, C₆₋₁₀ aryl, 5- to 10-membered heteroaryl or 4- to 10-membered heterocyclyl, wherein the alkyl, cycloalkyl, aryl, heteroaryl or heterocyclyl is optionally further substituted with 0 to 4 substituents selected from H, F, Cl, Br, I, OH, ═O, NH₂, CN, CF₃, COOH, C₁₋₄ alkyl, C₃₋₆ cycloalkyl, or C₁₋₄ alkoxy, and the heteroaryl or heterocyclyl contains 1 to 4 heteroatoms selected from O, S, or N;
each R^{b22} is independently selected from H, C₁₋₄ alkyl, C₁₋₄ alkenyl, C₁₋₄ alkynyl, C₁₋₄ alkoxy, or C₃₋₆ cycloalkyl, wherein the alkyl, alkoxy, cycloalkyl, alkenyl, or alkynyl is optionally further substituted with 0 to 4 substituents selected from H, F, Cl, Br, I, OH, ═O, NH₂, CN, CF₃, COOH, C₁₋₄ alkyl, C₃₋₆ cycloalkyl, or C₁₋₄ alkoxy;
K is selected from 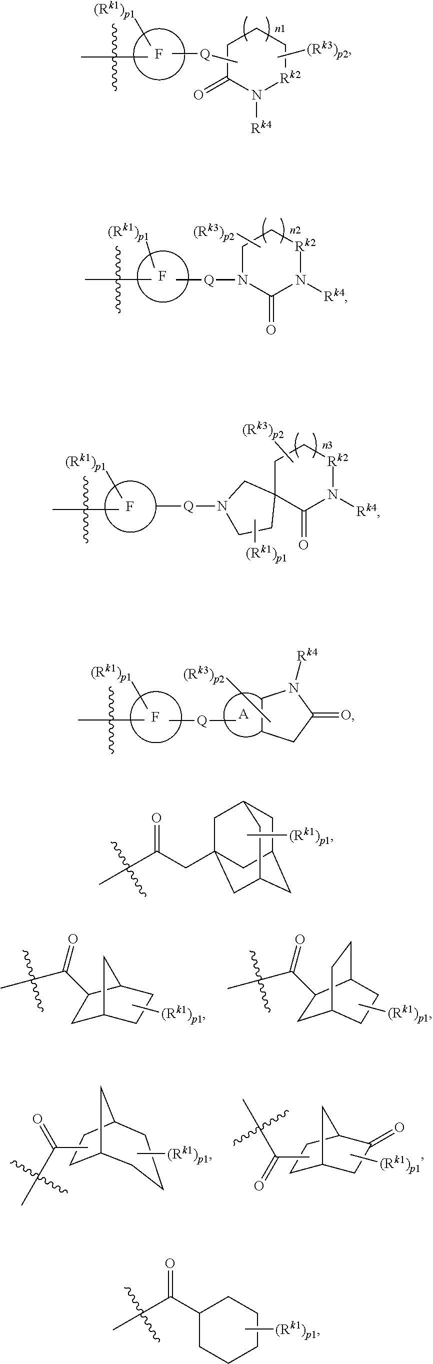 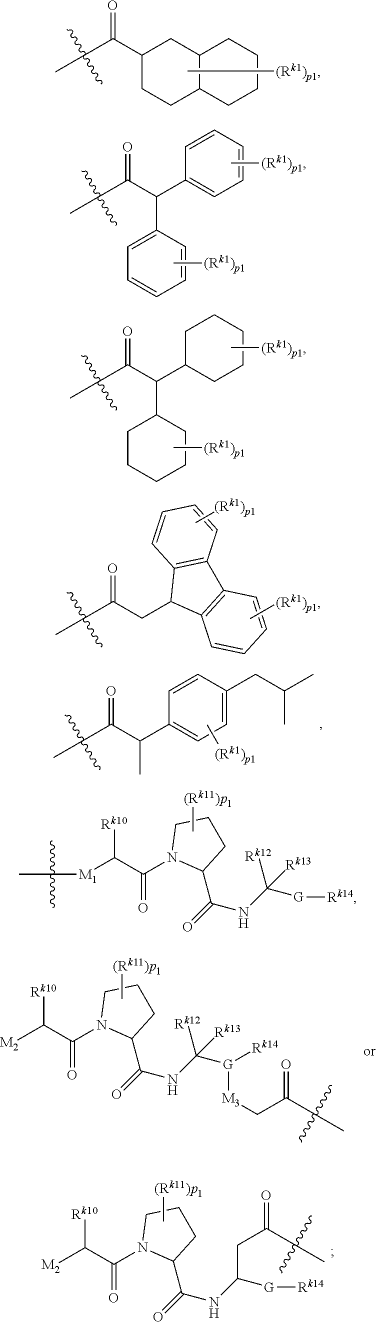
each Q is independently selected from a bond, —O—, —S—, —CH₂—, —NR^{q}—, —CO—, —NR^{q}CO—, —CONR^{q}— or 3- to 12-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S, or N;
R^{q} is selected from H or C₁₋₆ alkyl;
A is selected from C₃₋₁₀ carbocyclyl, C₆₋₁₀ aryl, 3- to 10-membered heterocyclyl or 5- to 10-membered heteroaryl, wherein the heterocyclyl or heteroaryl contains 1 to 4 heteroatoms selected from O, S, or N;
each F is independently selected from C₃₋₂₀ carbocyclyl, C₆₋₂₀ aryl, 3- to 20-membered heterocyclyl or 5- to 20-membered heteroaryl, wherein the heterocyclyl or heteroaryl contains 1 to 4 heteroatoms selected from O, S, or N;
each R^{k2} is independently selected from a bond, —CO—, —SO₂—, —SO— or —C(R^{k3})₂;
each R^{k1} is independently selected from H, F, Cl, Br, I, OH, ═O, NH₂, CN, COOH, CONH₂, C₁₋₆ alkyl or C₁₋₆ alkoxy, wherein the alkyl or alkoxy is optionally further substituted with 0 to 4 substituents selected from H, F, Cl, Br, I, OH, ═O, NH₂, CN, COOH, CONH₂, C₁₋₄ alkyl or C₁₋₄ alkoxy;
each R^{k3} is independently selected from H, F, Cl, Br, I, OH, ═O, NH₂, CN, COOH, CONH₂, C₁₋₆ alkyl, C₁₋₆ alkoxy, C₃₋₈ cycloalkyl or 3- to 8-membered heterocyclyl, wherein the alkyl, alkoxy, cycloalkyl or heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S, or N;
or two R^{k3} together with the carbon atoms or ring backbones to which they are directly attached form 3- to 8-membered carbocycle or 3- to 8-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S, or N;
each R^{k4} is independently selected from H, OH, NH₂, CN, CONH₂, C₁₋₆ alkyl, C₃₋₈ cycloalkyl or 3- to 8-membered heterocyclyl, wherein the alkyl, cycloalkyl or heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S, or N;
M₁ is selected from a bond, —CH₂—C(═O)NH— or —C(═O)CH₂NH—;
M₂ is selected from —NHC(═O)—C₁₋₆ alkyl, —NHC(═O)—C₃₋₆ cycloalkyl or 4- to 10-membered heterocyclyl, wherein the alkyl, cycloalkyl or heterocyclyl is optionally further substituted with 0 to 4 substituents selected from H, F, Cl, Br, I, ═O, OH, NH₂, C₁₋₄ alkyl or C₁₋₄ alkoxy, and the heterocyclyl contains 1 to 4 heteroatoms selected from O, S, or N;
M₃ is selected from —NH— or —O—;
R^{k10} is selected from C₁₋₆ alkyl, wherein the alkyl is optionally further substituted with 0 to 4 substituents selected from H, F, Cl, Br, I, ═O, OH, C₁₋₆ alkyl or C₃₋₆ cycloalkyl;
each R^{k11} is independently selected from H, F, Cl, Br, I, ═O, OH, SH, C₁₋₆ alkyl, C₁₋₆ alkoxy or C₁₋₆ alkylthio or —O—C(═O)—C₁₋₆ alkyl, wherein the alkyl, alkoxy or alkylthio is optionally further substituted with 0 to 4 substituents selected from H, F, Cl, Br, I, OH, C₁₋₄ alkyl or C₁₋₄ alkoxy;
R^{k12} and R^{k13} are each independently selected from H, C₁₋₆ alkyl or C₃₋₆ cycloalkyl, wherein the alkyl or cycloalkyl is optionally further substituted with 0 to 4 substituents selected from H, F, Cl, Br, I, ═O, OH, NH₂, C₁₋₄ alkyl or C₁₋₄ alkoxy;
R^{k14} is selected from 5- to 6-membered heteroaryl, wherein the heteroaryl is optionally further substituted with 0 to 4 substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 heteroatoms selected from N, O, or S;
G is selected from 6- to 10-membered aryl or 5- to 10-membered heteroaryl, wherein the aryl or heteroaryl is optionally further substituted with 0 to 4 substituents selected from H, F, Cl, Br, I, OH, ═O, CF₃, CN, C₁₋₄ alkyl, halogen-substituted C₁₋₄ alkyl, hydroxyl-substituted C₁₋₄ alkyl, C₁₋₄ alkoxy or C₃₋₆ cycloalkyl, and the heteroaryl contains 1 to 4 heteroatoms selected from N, O, or S;
n, r, and t are each independently selected from 0, 1, 2, 3 or 4;
m1, m2, m3, n1, n2, and n3 are each independently selected from 0, 1, 2 or 3;
each p1 or p2 is independently selected from 0, 1, 2, 3, 4 or 5.

2. The compound or the stereoisomer, deuterated compound, solvate, prodrug, metabolite, pharmaceutically acceptable salt or co-crystal thereof according to claim 1, wherein
L is selected from -Cy1-Ak1-Cy2-Ak2-Cy3-Ak3-Cy4-Ak4-;
Ak1, Ak2, Ak3 and Ak4 are each independently selected from —(CH₂)_{q}—, O, —(CH₂)_{q}—NR^{L}, NR^{L}C(═O), C(═O), C(═O)—(CH₂)_{q}—NR^{L}, C≡C or a bond;
each q is independently selected from 0, 1, 2, 3 or 4;
each R^{L} is independently selected from H or C₁₋₆ alkyl;
Cy1, Cy2, Cy3 and Cy4 are each independently selected from a bond, 4- to 7-membered mono-heterocyclic ring, 4- to 10-membered fused heterocyclic ring, 5- to 12-membered spiro-heterocyclic ring, 7- to 10-membered bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the aryl, heteroaryl, cycloalkyl, mono-heterocyclic ring, fused heterocyclic ring, spiro-heterocyclic ring or bridged-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the heteroaryl, mono-heterocyclic ring, fused heterocyclic ring, spiro-heterocyclic ring or bridged-heterocyclic ring contains 1 to 4 heteroatoms selected from O, S, or N;
provided that at least one of Cy1, Cy2, Cy3, and Cy4 cannot be a bond, and when three of them are selected from a bond, the remaining one cannot be selected from triazolyl.

3. The compound or the stereoisomer, deuterated compound, solvate, prodrug, metabolite, pharmaceutically acceptable salt or co-crystal thereof according to claim 1, wherein
Cy1, Cy2, Cy3 and Cy4 are each independently selected from a bond, 4- to 7-membered nitrogen-containing mono-heterocyclic ring, 4- to 10-membered nitrogen-containing fused heterocyclic ring, 5- to 12-membered nitrogen-containing spiro-heterocyclic ring, 7- to 10-membered nitrogen-containing bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the mono-heterocyclic ring, fused heterocyclic ring, bridged-heterocyclic ring, spiro-heterocyclic ring, cycloalkyl, aryl or heteroaryl is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring, spiro-heterocyclic ring or heteroaryl contains 1 to 4 heteroatoms selected from O, S, or N;
each R^{L} is independently selected from H or C₁₋₄ alkyl;
each R^{w1} is independently selected from H, methyl, ethyl, isopropyl, cyclopropyl or cyclobutyl;
each R^{w2} or R^{w3} is independently selected from H, F, Cl, Br, I, OH, NH₂, CN, COOH, CONH₂, methyl, ethyl, isopropyl, ethenyl, ethynyl, methoxy, ethoxy, propoxy, cyclopropyl or cyclobutyl, wherein the methyl, ethyl, isopropyl, ethenyl, ethynyl, methoxy, ethoxy, propoxy, cyclopropyl or cyclobutyl is optionally further substituted with 0 to 4 substituents selected from H, F, Cl, Br, I, OH, or NH₂;
alternatively, R^{w2} and R^{w3} together with the carbon atoms to which they are attached form cyclopropyl, cyclobutyl, cyclopentyl, azetidinyl, oxetanyl, pyrrolidinyl, or piperidyl, wherein the cyclopropyl, cyclobutyl, cyclopentyl, azetidinyl, oxetanyl, pyrrolidinyl, or piperidyl is optionally further substituted with 0 to 4 substituents selected from H, F, Cl, Br, I, OH, NH₂, CN, CF₃, or CHF₂;
each R^{b1} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, deuterated methoxy, methylthio, ethylthio, morpholinyl, piperazinyl, pyrrolidinyl, piperidyl or oxazolidinyl, wherein the methoxy, ethoxy, propoxy, isopropyloxy, methylthio, or ethylthio is optionally further substituted with 0 to 4 substituents selected from H, F, Cl, Br, I, OH, or NH₂;
each R^{b2} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, morpholinyl, piperazinyl, pyrrolidinyl, piperidyl or oxazolidinyl, wherein the methyl, ethyl, ethenyl, ethynyl, methoxy, ethoxy, propoxy, isopropyloxy, morpholinyl, piperazinyl, pyrrolidinyl, piperidyl or oxazolidinyl is optionally further substituted with 0 to 4 substituents selected from H, F, Cl, Br, I, OH, or NH₂;
each R^{b3} is independently selected from H, F, Cl, Br, I, ═O, CF₃, CHF₂, OH, CN, COOH, NH₂, CONH₂, S(═O)₂NH₂, S(═O)₂CH₃, methyl, ethyl, isopropyl, ethenyl, ethynyl, methoxy, ethoxy, propoxy, methylthio or ethylthio, wherein the methyl, ethyl, isopropyl, ethenyl, ethynyl, methoxy, ethoxy, propoxy, methylthio or ethylthio is optionally further substituted with 0 to 4 substituents selected from H, F, Cl, Br, I, OH, or NH₂;
r is selected from 1 or 2;
each t is independently selected from 0, 1 or 2;
K is selected from 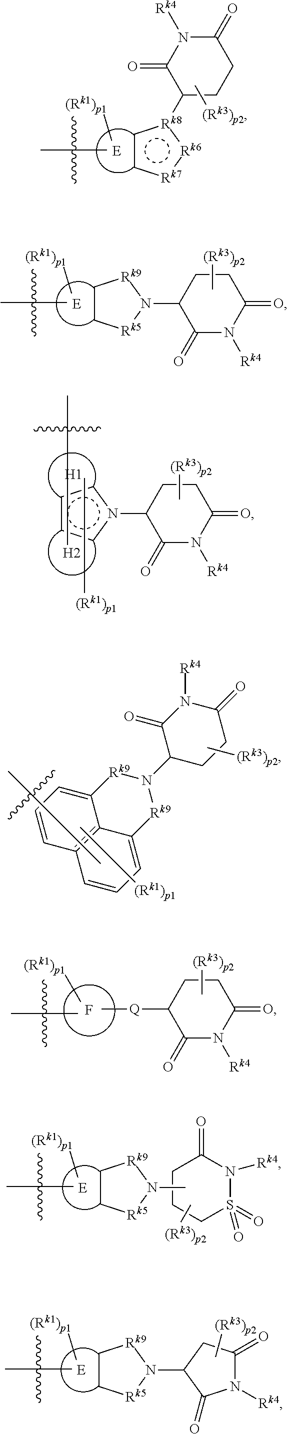 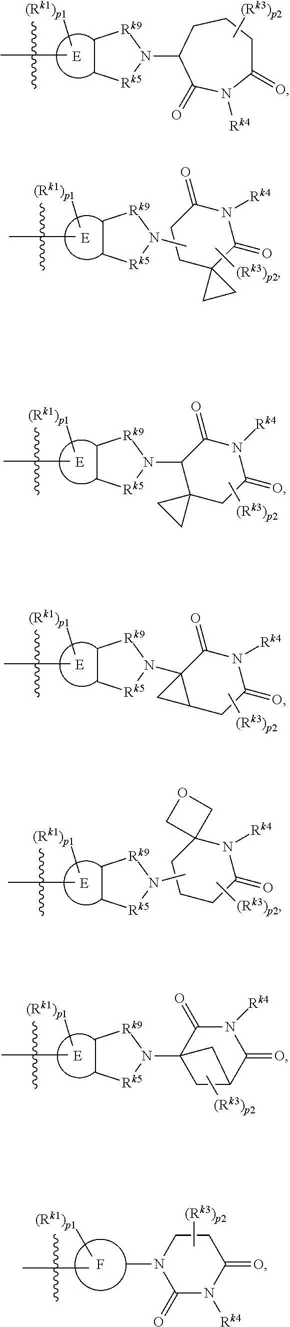 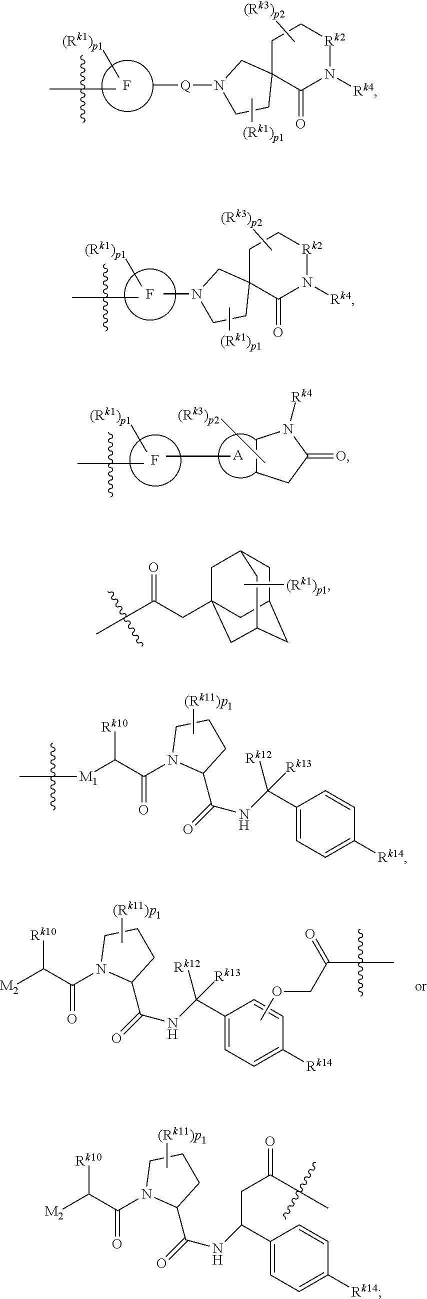
or K is selected from 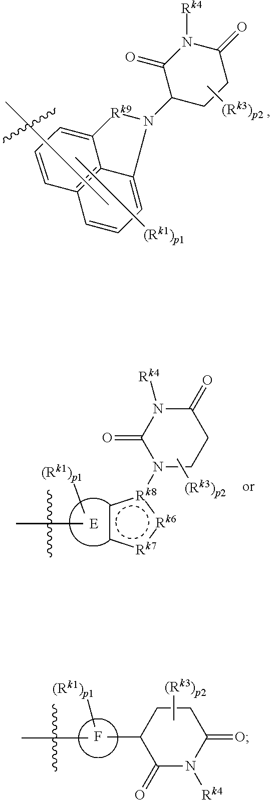 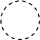
represents a ring selected from an aromatic ring or a non-aromatic ring;
each Q is independently selected from —O—, —S—, —CH₂—, —NR^{q}—, —CO—, —NR^{q}CO—, —CONR^{q}— or 4- to 7-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S, or N;
R^{q} is selected from H or C₁₋₄ alkyl;
R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 substituents selected from H, F, Cl, Br, I, OH, or NH₂;
or two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S, or N;
each R^{k4} is independently selected from H, OH, NH₂, CF₃, CN, or C₁₋₄ alkyl;
each R^{k5} is independently selected from CO, CH₂, SO₂ or 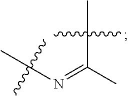 [SMILES: */N=C(\*)C]
each R^{k6} is independently selected from CO, CH, SO, SO₂, CH₂ or N;
each R^{k7} is independently selected from CO, CH, N, CH₂, O, S, N(CH₃) or NH;
each R^{k8} is independently selected from C, N or CH;
each R^{k9} is independently selected from CO, CH₂ or SO₂;
each A, H1 or H2 is independently selected from C₃₋₈ carbocycle, a benzene ring, 4- to 7-membered heterocycle or 5- to 6-membered heteroaryl, wherein the heterocycle or heteroaryl contains 1 to 4 heteroatoms selected from O, S, or N;
each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 heteroatoms selected from O, S, or N;
each F is independently selected from 3- to 7-membered monocycloalkyl, 4- to 10-membered fused cycloalkyl, 5- to 12-membered spiro cycloalkyl, 5- to 10-membered bridged cycloalkyl, 4- to 7-membered mono-heterocyclic ring, 4- to 10-membered fused heterocyclic ring, 5- to 12-membered spiro-heterocyclic ring, 5- to 10-membered bridged-heterocyclic ring, C₆₋₁₄ aryl or 5- to 10-membered heteroaryl, wherein the mono-heterocyclic ring, fused heterocyclic ring, spiro-heterocyclic ring, bridged-heterocyclic ring or heteroaryl contains 1 to 4 heteroatoms selected from O, S, or N.

4. The compound or the stereoisomer, deuterated compound, solvate, prodrug, metabolite, pharmaceutically acceptable salt or co-crystal thereof according to claim 3, wherein
R^{L} is selected from H, methyl or ethyl;
each q is independently selected from 0, 1 or 2;
Cy1, Cy2, Cy3 and Cy4 are each independently selected from a bond or one of the following substituted or unsubstituted groups: cyclopropyl, cyclobutyl, cyclopentyl, cyclohexyl, azetidinyl, azacyclopentyl, piperidine, azacyclohexenyl, morpholine, piperazine, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl-spiro-piperidine, azetidinyl-spiro-piperidine, azacyclopentyl-spiro-piperidine, azacyclohexyl-spiro-piperidine 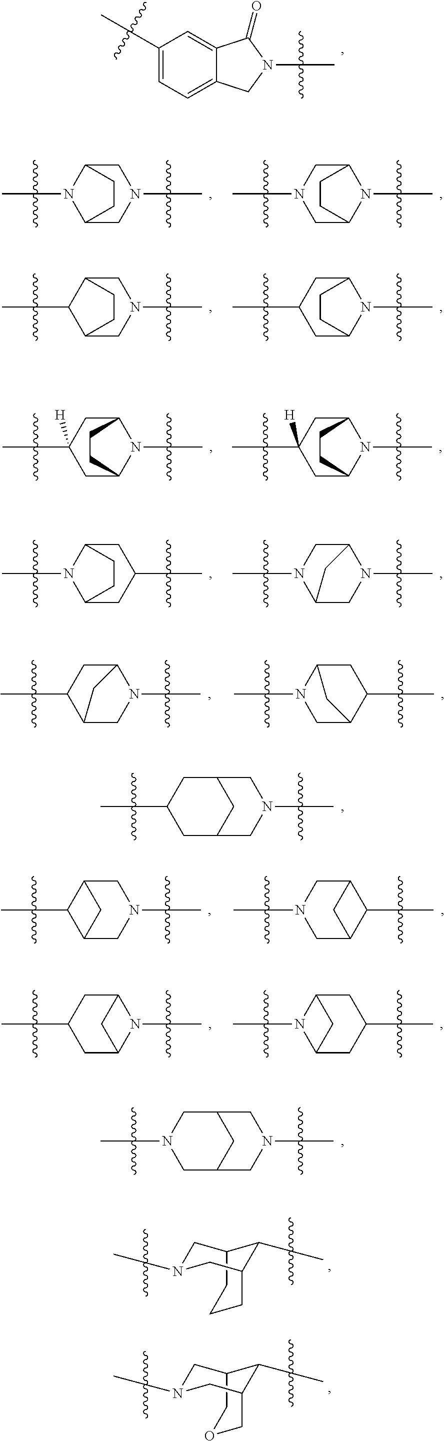 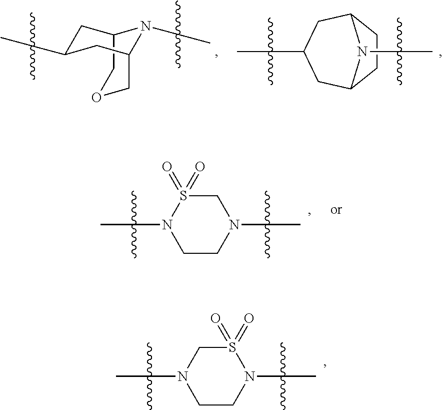 [SMILES: *C1CC2COCC(C1)N2* ; *C1CC2CCC(C1)N2* ; *N1CCN(*)S(=O)(=O)C1 ; *N1CCN(*)S(=O)(=O)C1]
which, when substituted, is optionally further substituted with 0 to 4 substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl or C₁₋₄ alkoxy;
B is selected from 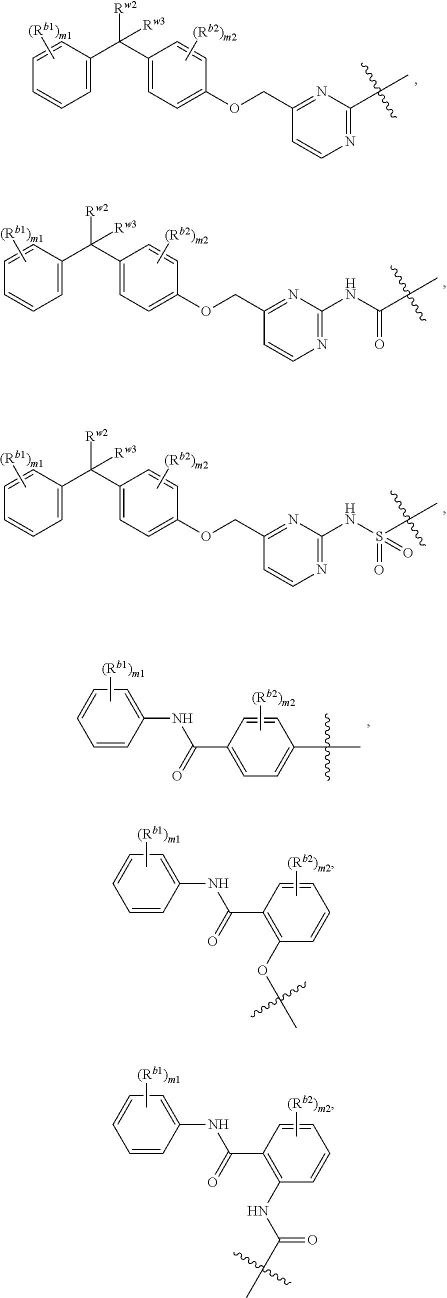 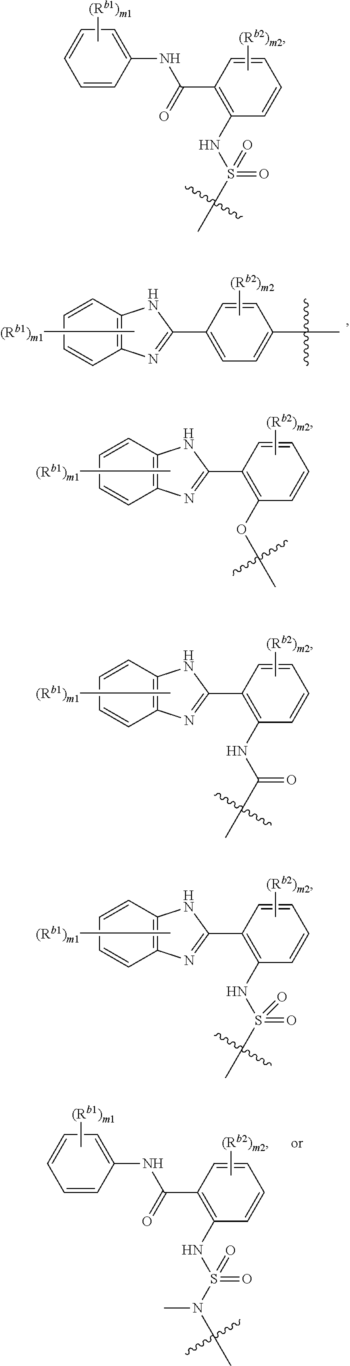 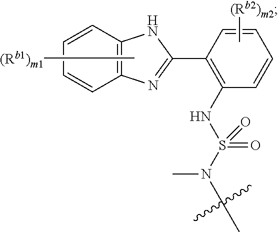
or B is selected from 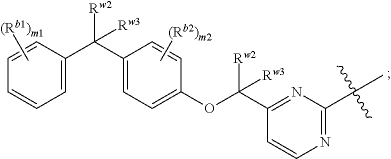
K is selected from 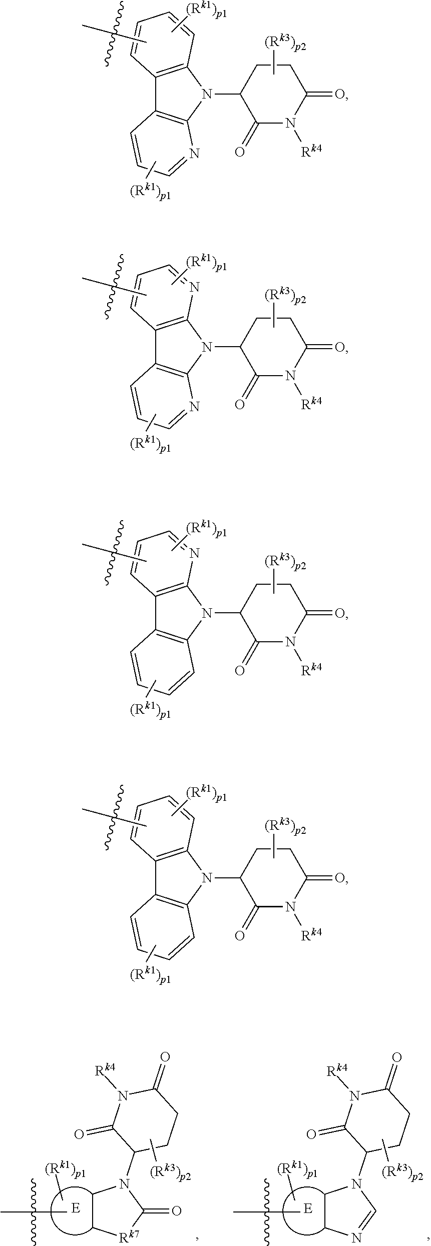 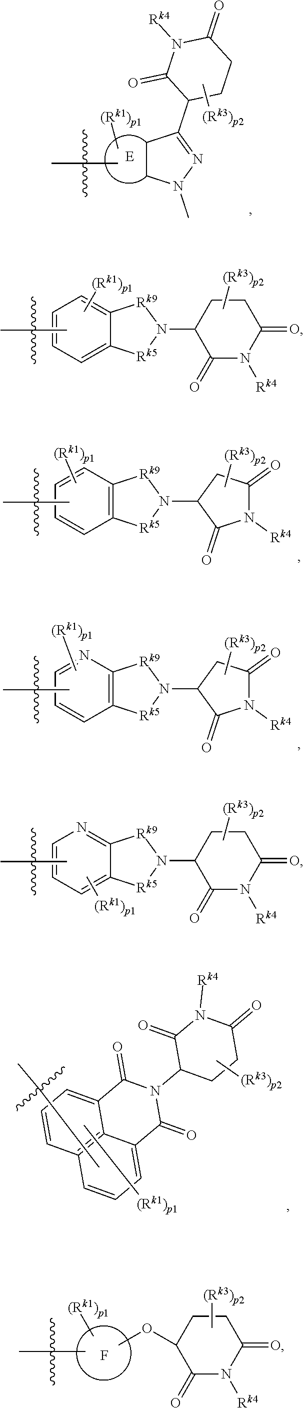 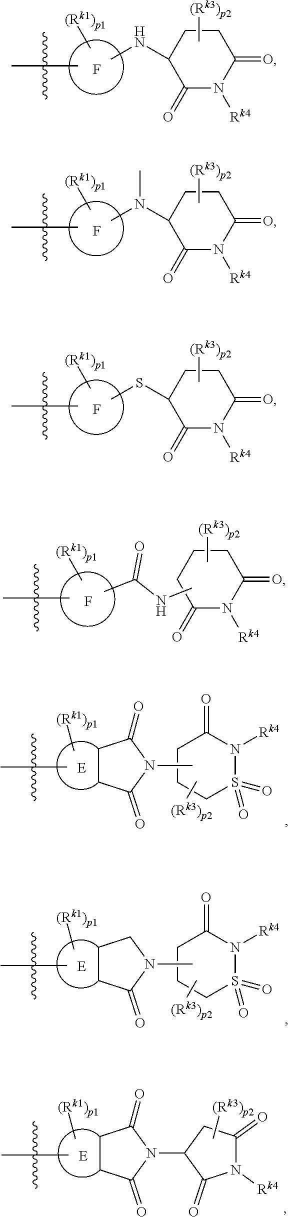 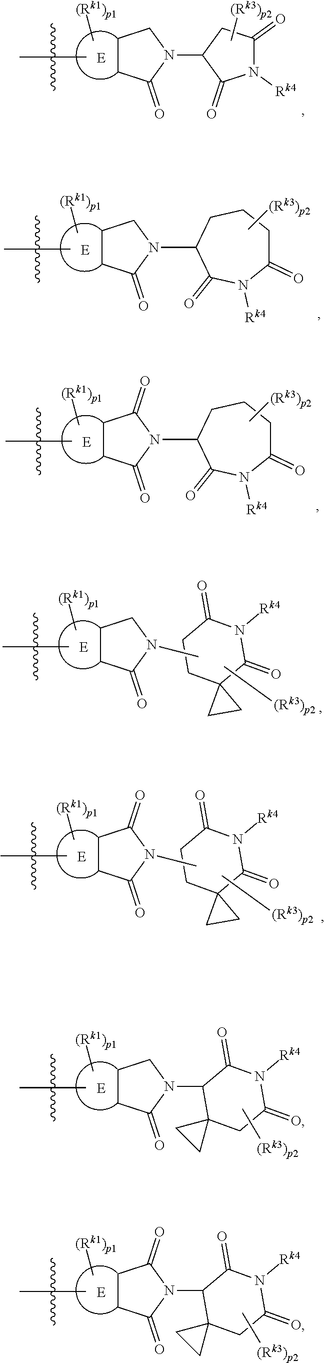 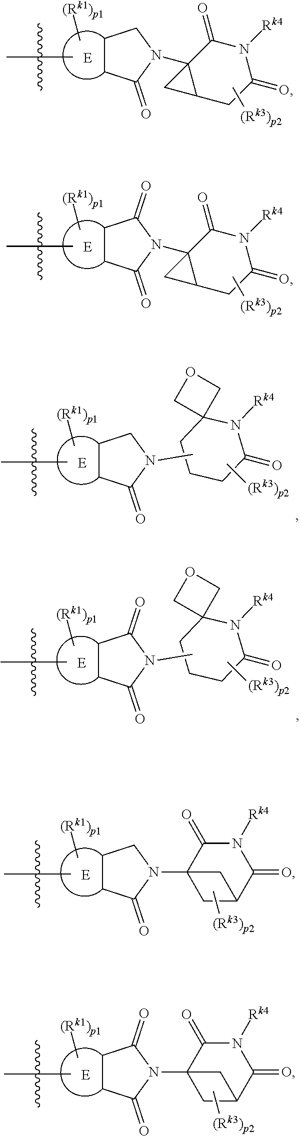 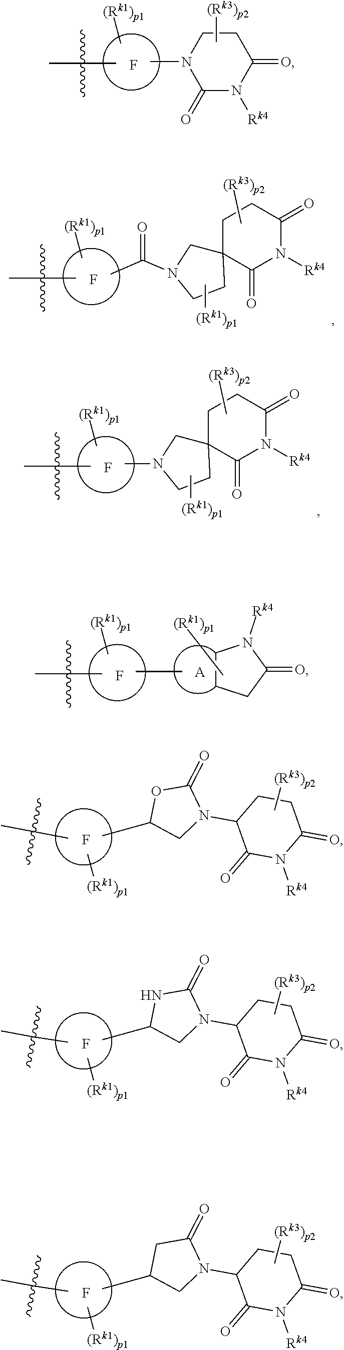 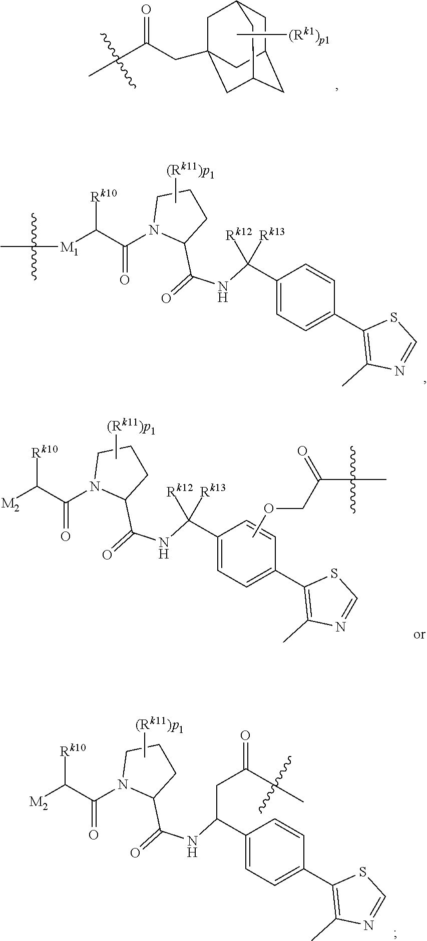
or K is selected from 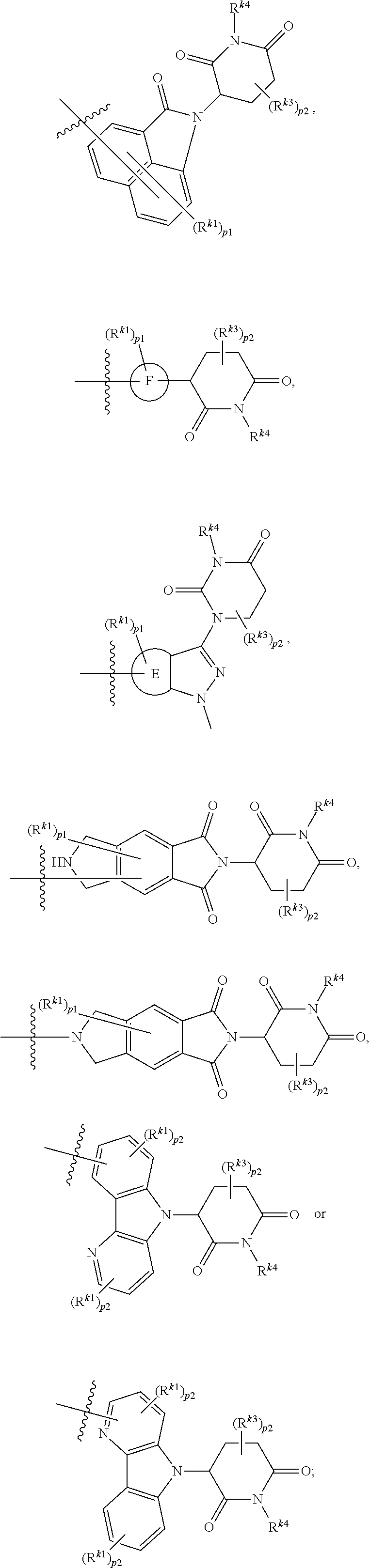
each E is independently selected from phenyl, pyridyl, pyridazinyl, pyrazinyl, pyrimidinyl, pyrrolyl, pyrazolyl, imidazoly, thiazolyl, furyl, thienyl or oxazolyl;
each A is independently selected from phenyl, pyridyl, pyridazinyl, pyrazinyl, pyrimidinyl, pyrrolyl, pyrazolyl, imidazoly, thiazolyl, furyl, thienyl or oxazolyl;
each F is independently selected from cyclopropyl, cyclobutyl, cyclopentyl, cyclohexyl, phenyl, naphthyl, anthryl, phenanthrenyl, azetidinyl, azacyclopentyl, piperidyl, morpholinyl, pyridyl, pyrimidinyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidinyl, benzopyridazinyl, pyrrolopyrrolyl, pyrrolopyridinyl, pyrrolopyrimidinyl, pyrrolopyridazinyl, pyrrolopyrazinyl, imidazopyrimidinyl, imidazopyridinyl, imidazopyrazinyl, imidazopyridazinyl, pyrazolopyridinyl, pyrazolopyrimidinyl, pyrazolopyridazinyl, pyrazolopyrazinyl, pyrimidopyridyl, pyrimidopyrazinyl, pyrimidopyridazinyl, pyrimidopyrimidinyl, pyridopyridyl, pyridopyrazinyl, pyridopyridazinyl, pyridazopyridazinyl, pyridazopyrazinyl or pyrazinopyrazinyl;
each R^{k7} is independently selected from CH₂, O, N(CH₃) or NH;
each p1 or p2 is independently selected from 0, 1 or 2.

5. The compound or the stereoisomer, deuterated compound, solvate, prodrug, metabolite, pharmaceutically acceptable salt or co-crystal thereof according to claim 4, wherein
Cy1, Cy2, Cy3 and Cy4 are each independently selected from a bond or one of the following substituted or unsubstituted groups: 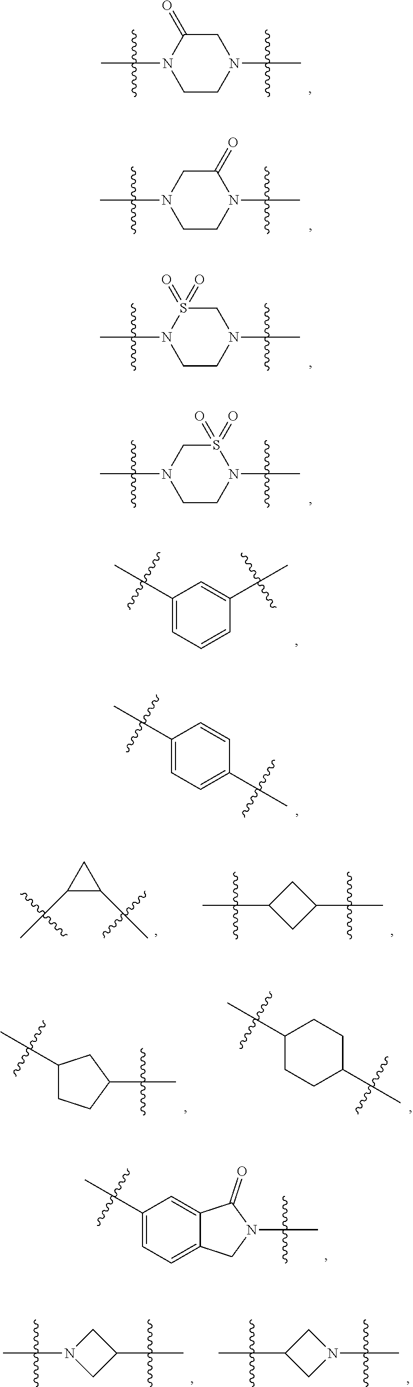 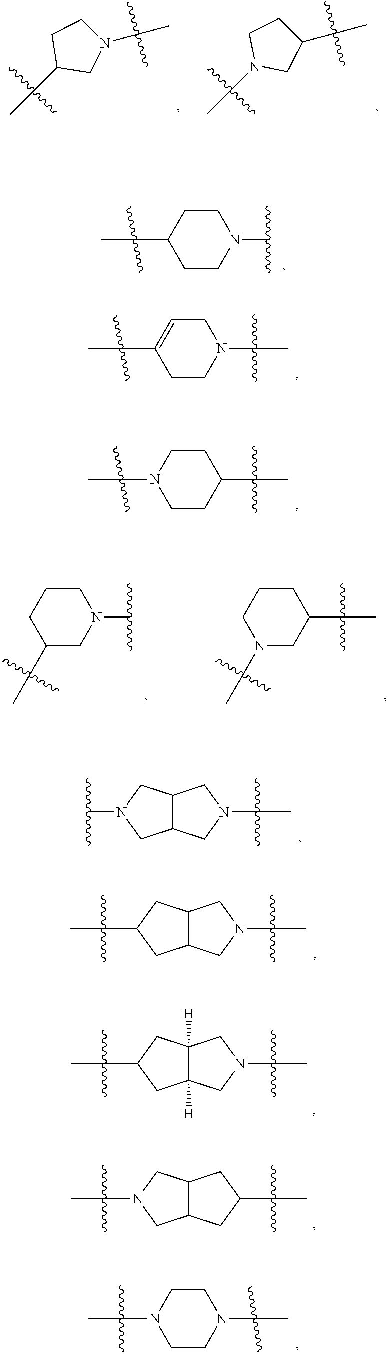 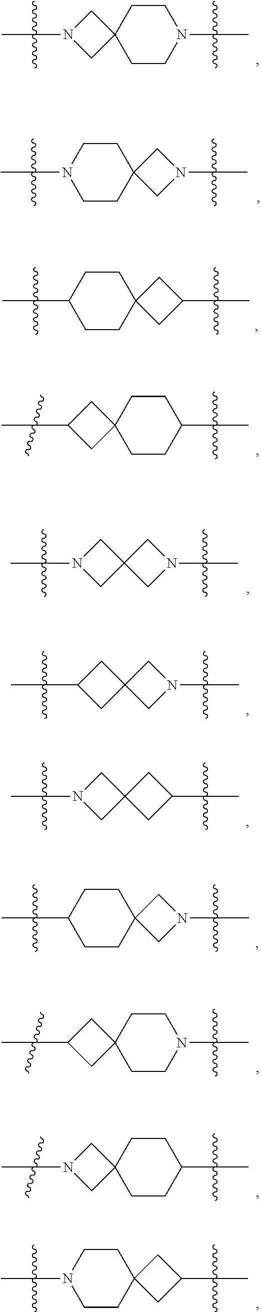 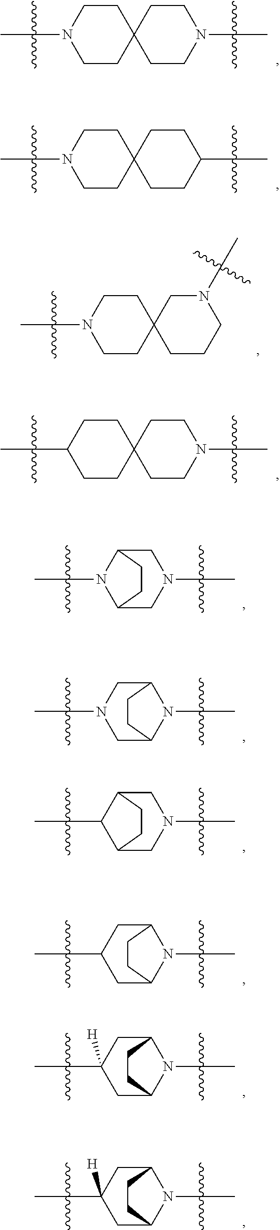 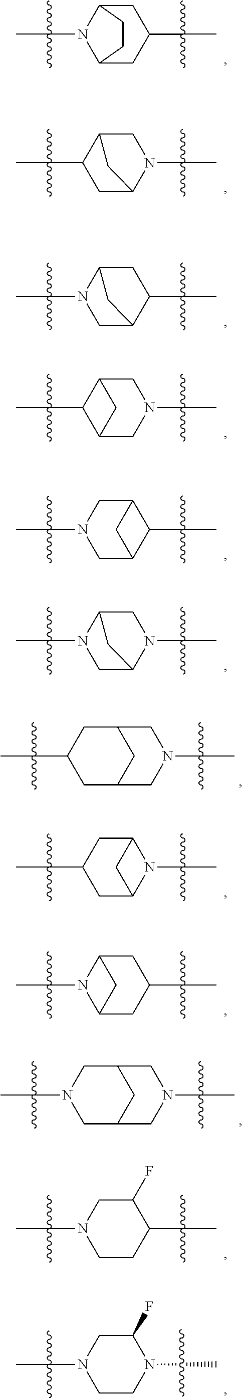 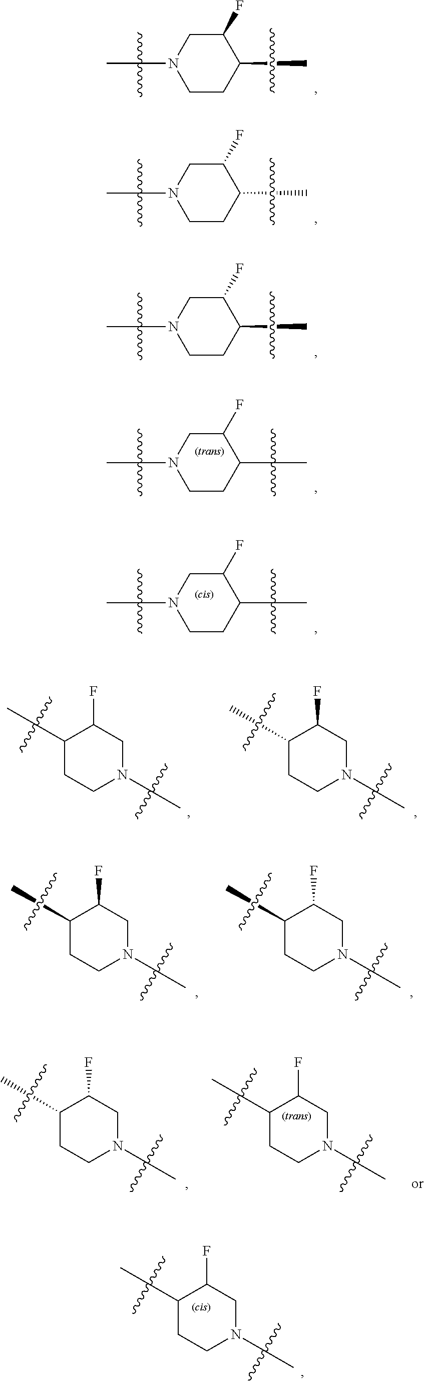
which, when substituted, is optionally further substituted with 0 to 4 substituents selected from H, F, CF₃, methyl, ═O, hydroxymethyl, COOH, CN or NH₂;
K is selected from 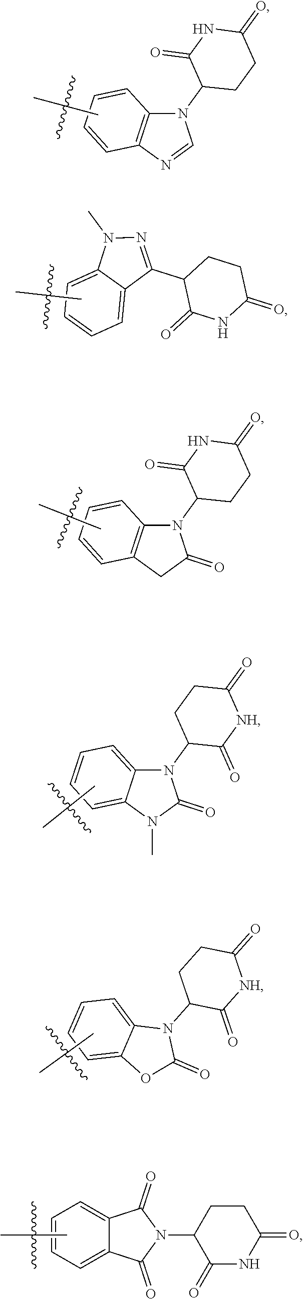 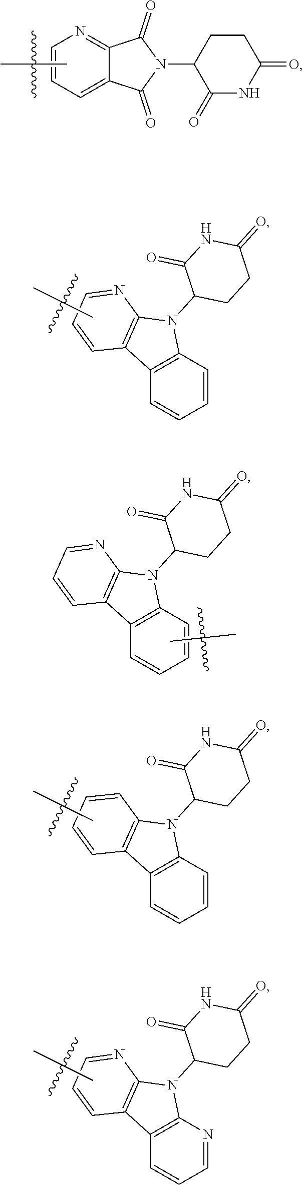 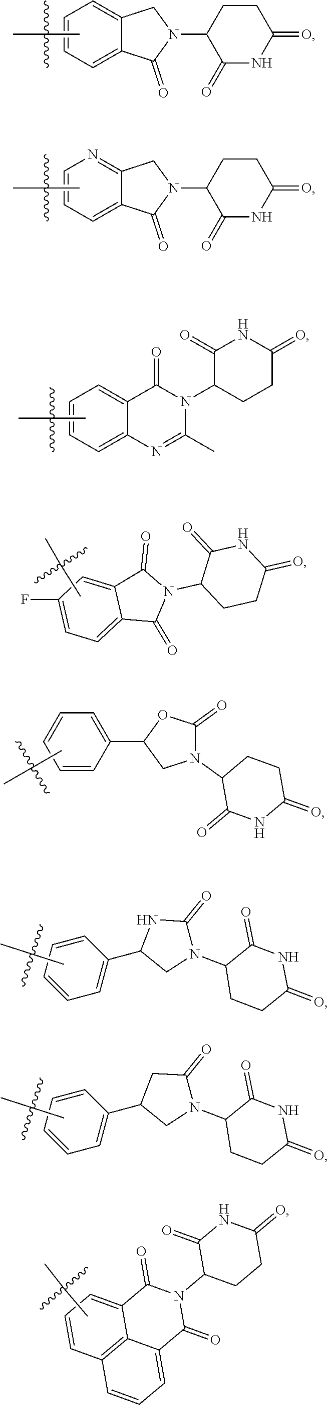 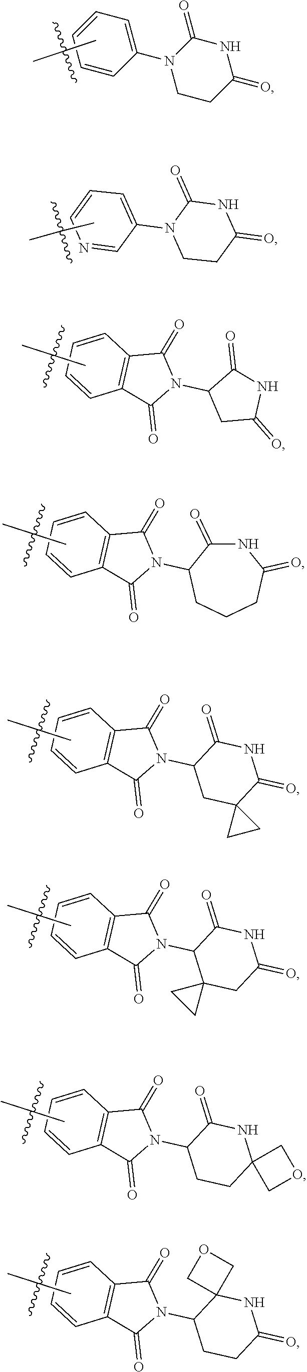 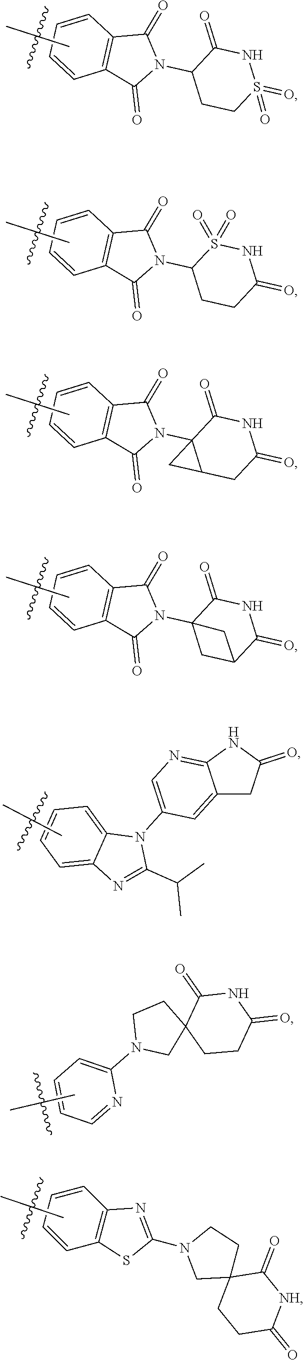 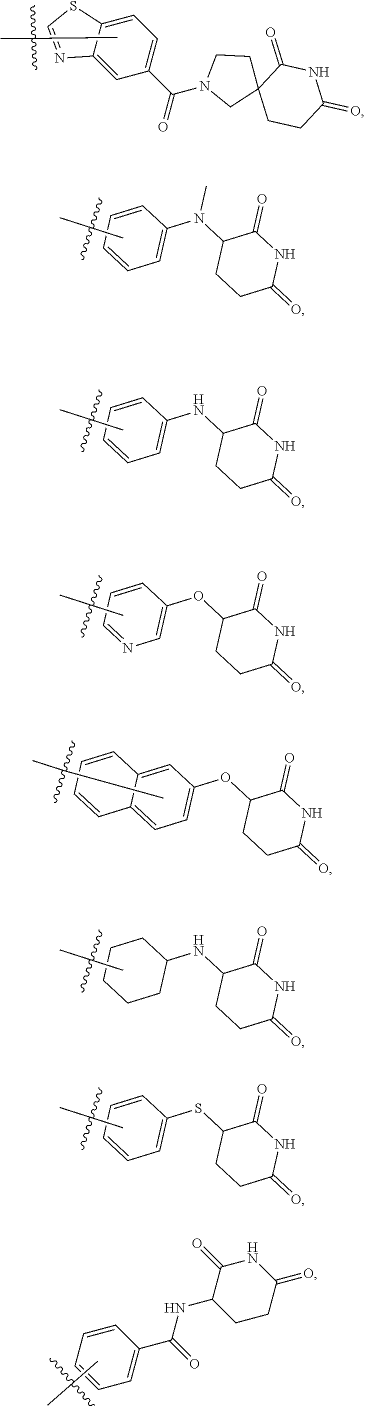 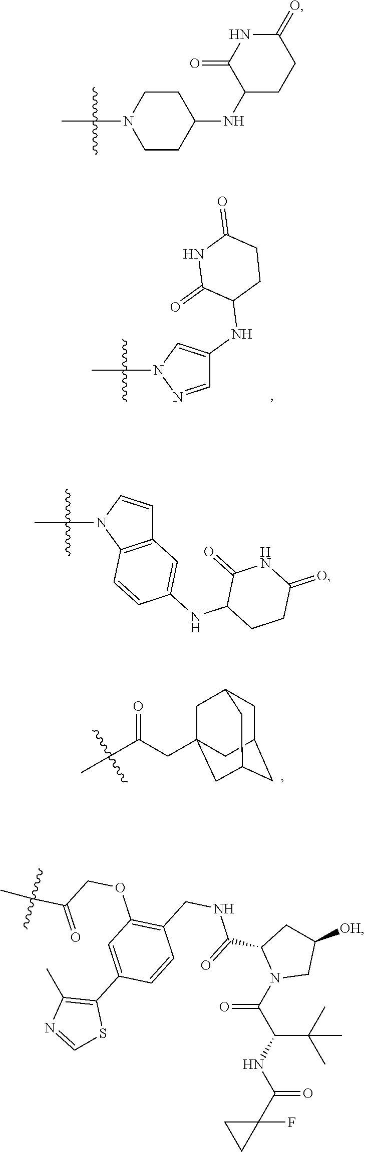 [SMILES: *N1CCC(NC2CCC(=O)NC2=O)CC1 ; *n1cc(NC2CCC(=O)NC2=O)cn1 ; *n1ccc2cc(NC3CCC(=O)NC3=O)ccc21 ; *C(=O)CC12CC3CC(CC(C3)C1)C2 ; *C(=O)COc1cc(-c2scnc2C)ccc1CNC(=O)[C@@H]1C[C@@H](O)CN1C(=O)[C@@H](NC(=O)C1(F)CC1)C(C)(C)C] 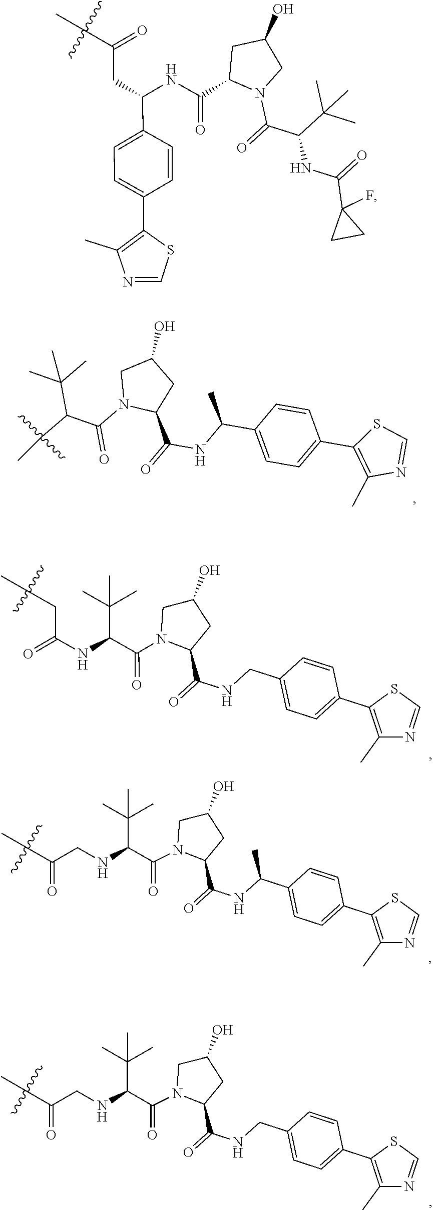 [SMILES: *C(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; *C(C(=O)N1C[C@H](O)C[C@H]1C(=O)N[C@@H](C)c1ccc(-c2scnc2C)cc1)C(C)(C)C ; *CC(=O)N[C@H](C(=O)N1C[C@H](O)C[C@H]1C(=O)NCc1ccc(-c2scnc2C)cc1)C(C)(C)C ; *C(=O)CN[C@H](C(=O)N1C[C@H](O)C[C@H]1C(=O)N[C@@H](C)c1ccc(-c2scnc2C)cc1)C(C)(C)C ; *C(=O)CN[C@H](C(=O)N1C[C@H](O)C[C@H]1C(=O)NCc1ccc(-c2scnc2C)cc1)C(C)(C)C] 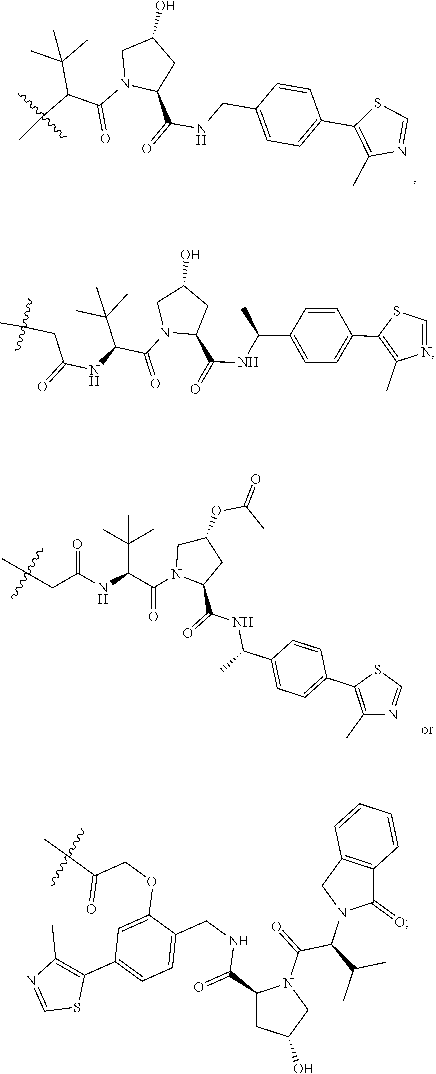 [SMILES: *C(C(=O)N1C[C@H](O)C[C@H]1C(=O)NCc1ccc(-c2scnc2C)cc1)C(C)(C)C ; *CC(=O)N[C@H](C(=O)N1C[C@H](O)C[C@H]1C(=O)N[C@@H](C)c1ccc(-c2scnc2C)cc1)C(C)(C)C ; *CC(=O)N[C@H](C(=O)N1C[C@H](OC(C)=O)C[C@H]1C(=O)N[C@@H](C)c1ccc(-c2scnc2C)cc1)C(C)(C)C ; *C(=O)COc1cc(-c2scnc2C)ccc1CNC(=O)[C@@H]1C[C@@H](O)CN1C(=O)[C@H](C(C)C)N1Cc2ccccc2C1=O]
or K is selected from

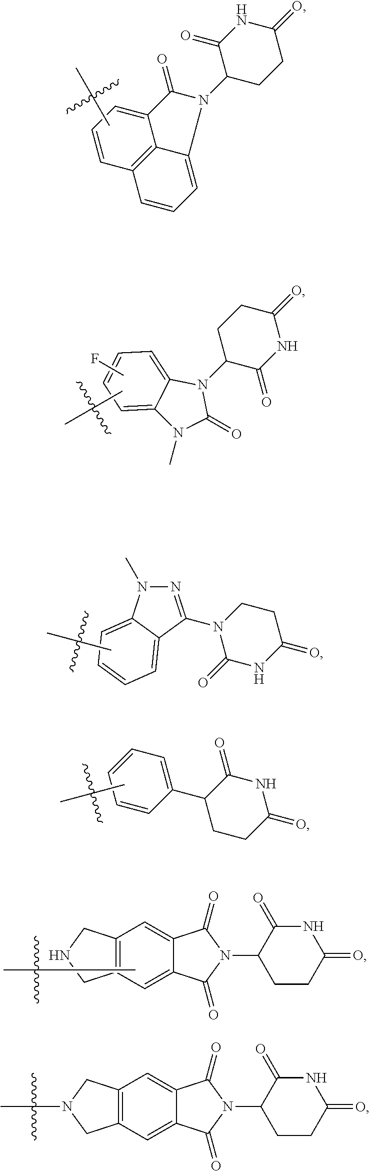

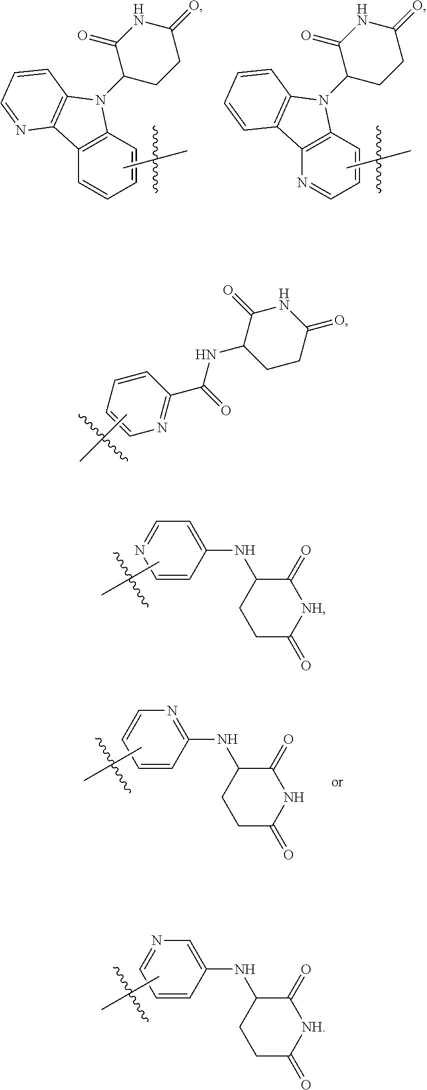

6. The compound or the stereoisomer, deuterated compound, solvate, prodrug, metabolite, pharmaceutically acceptable salt or co-crystal thereof according to claim 5, wherein
L is selected from a bond, 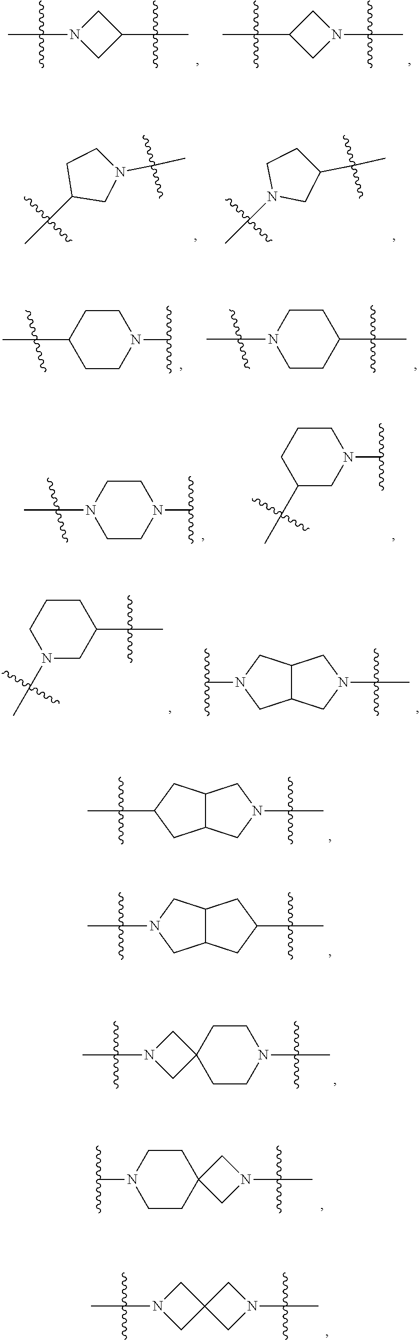 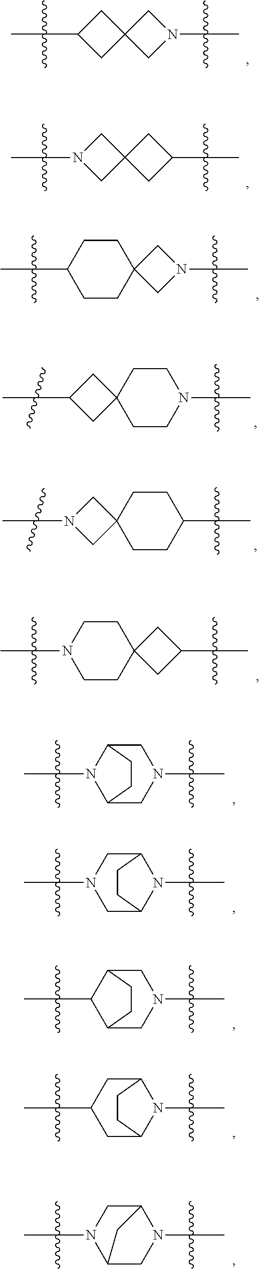 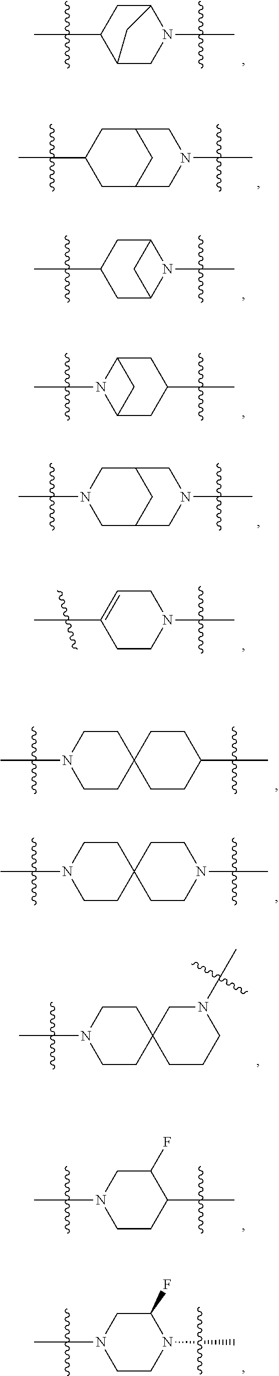 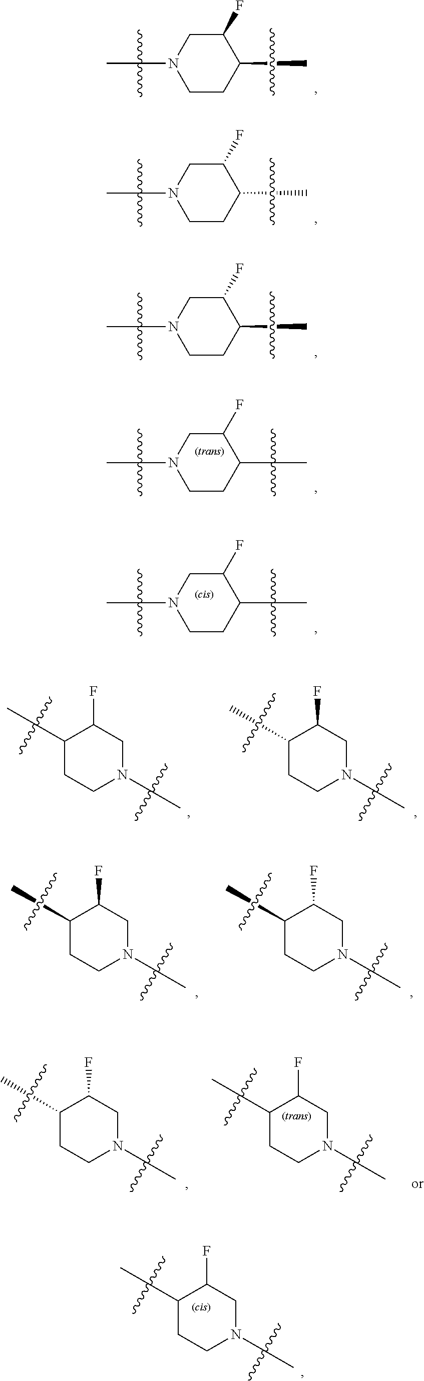
wherein the left side thereof is linked to B;
or L is selected from 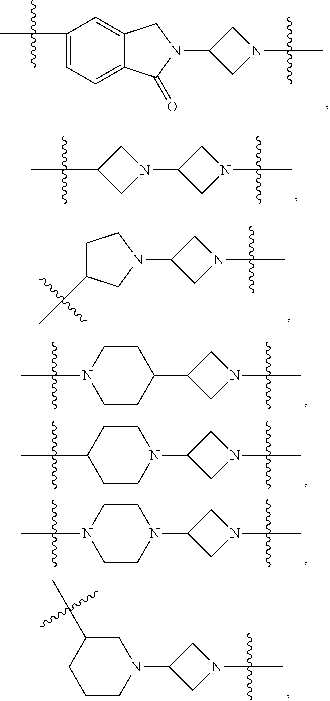 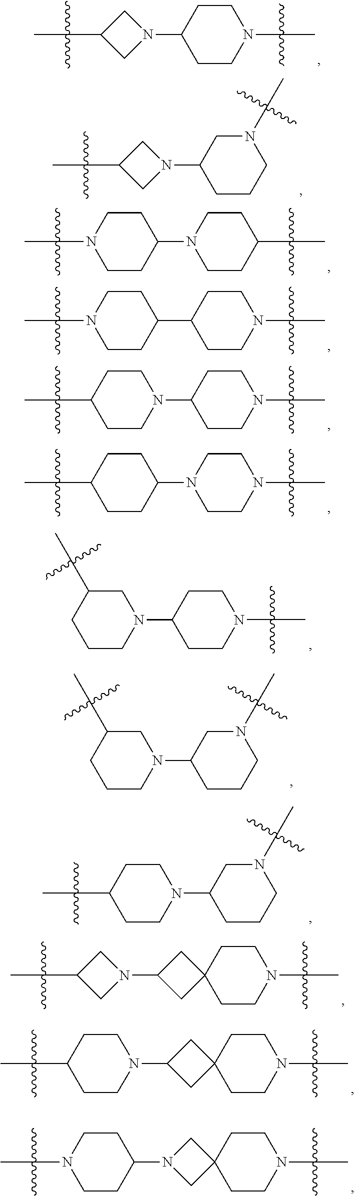 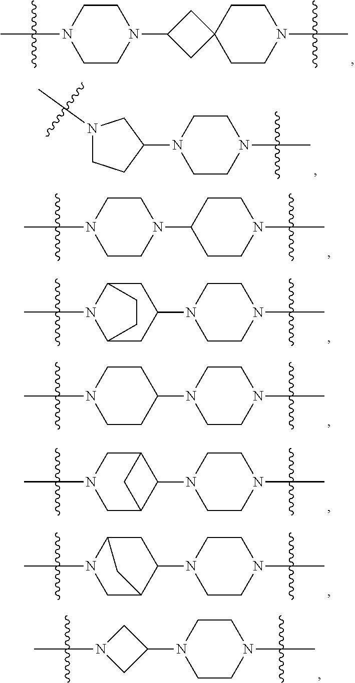 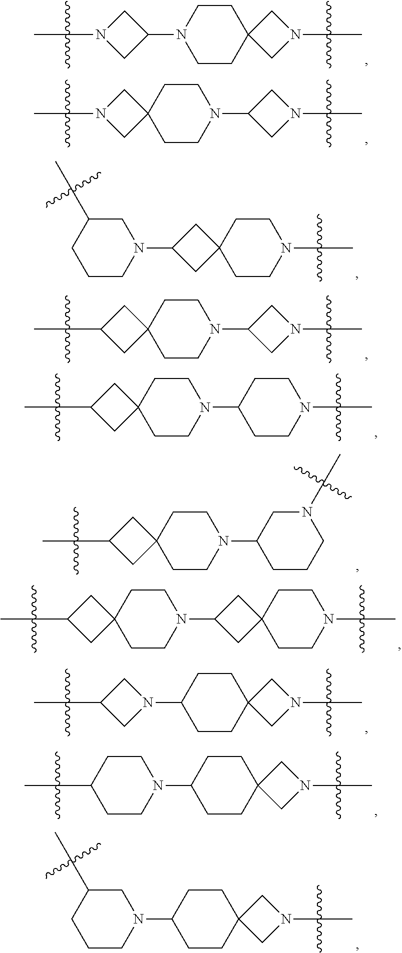 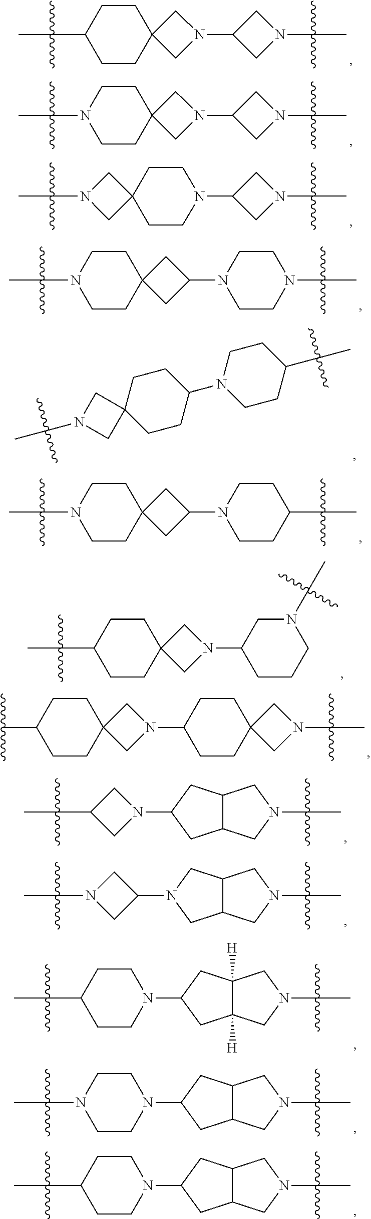 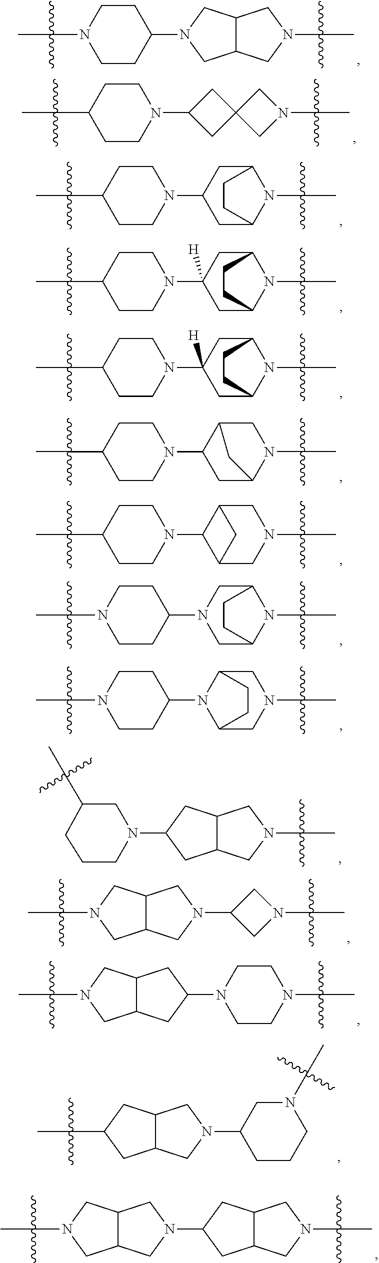 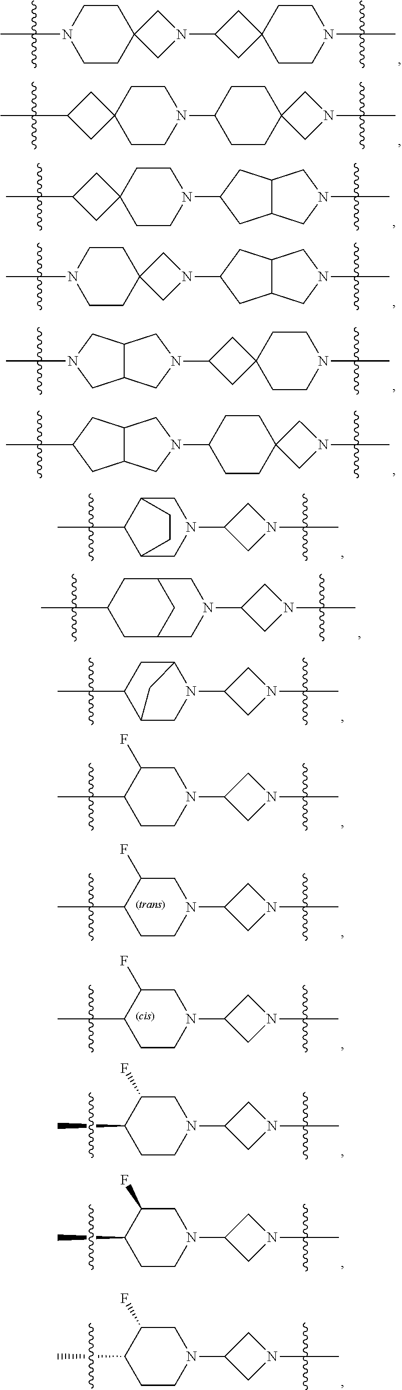 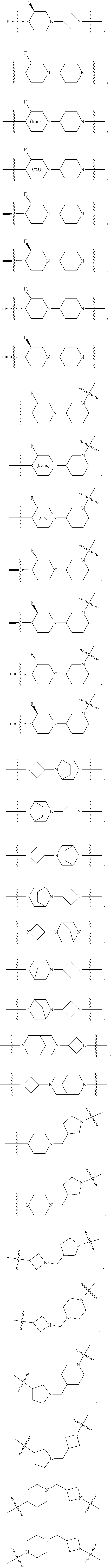 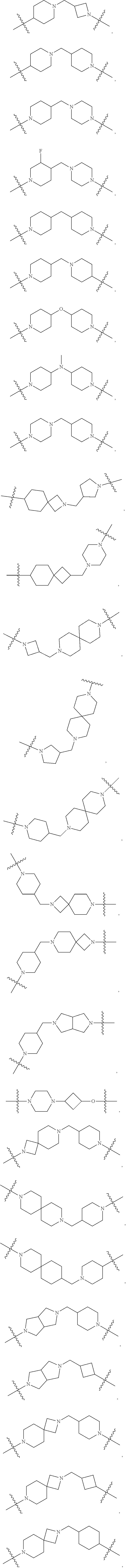 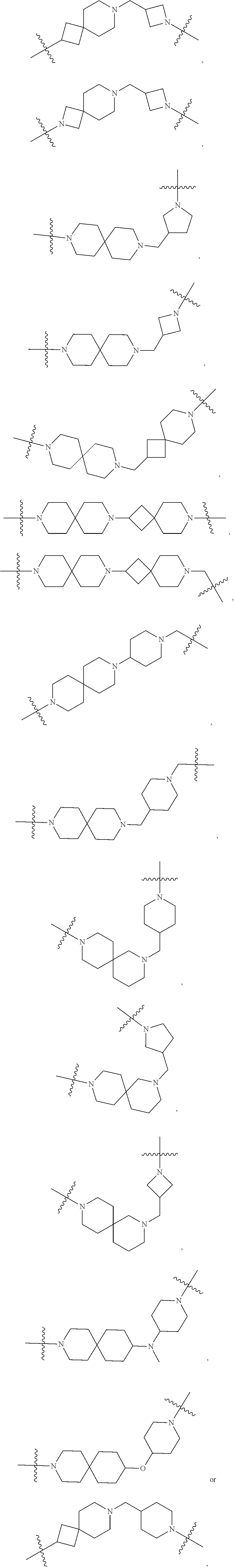
wherein the left side thereof is linked to B;
or L is selected from 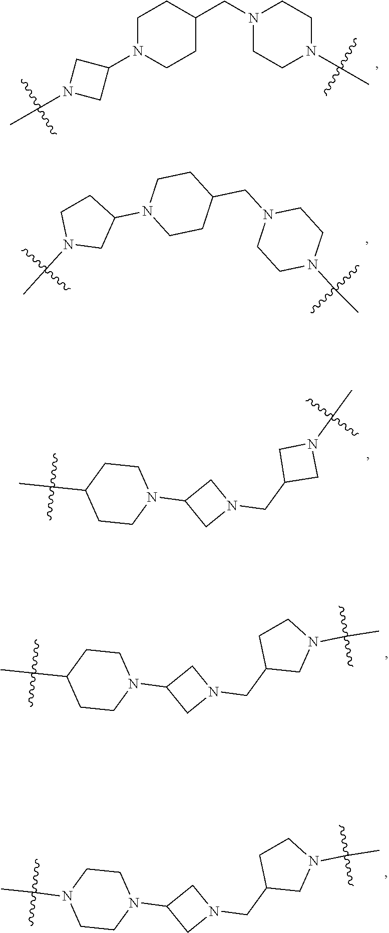 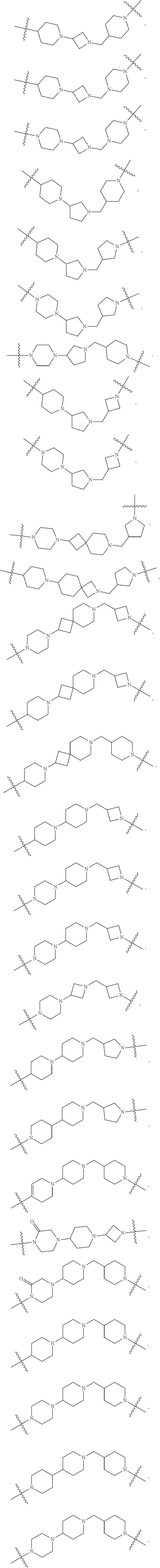 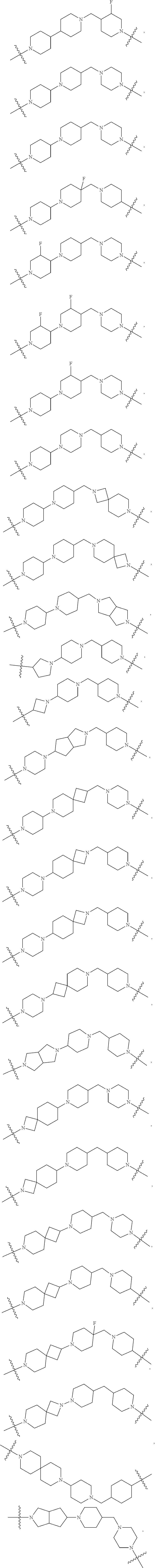 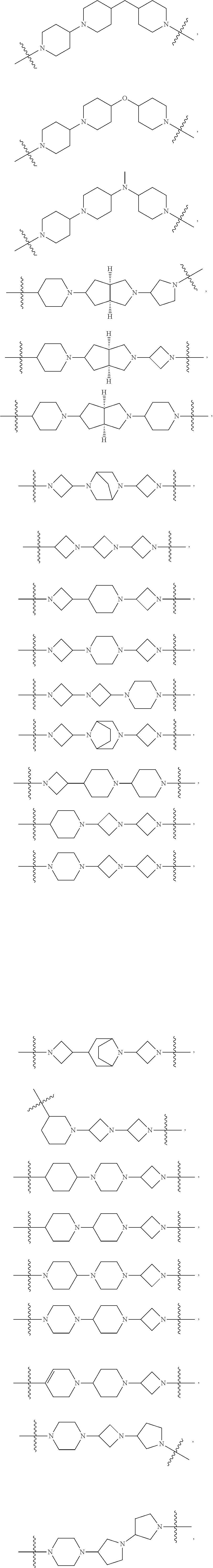 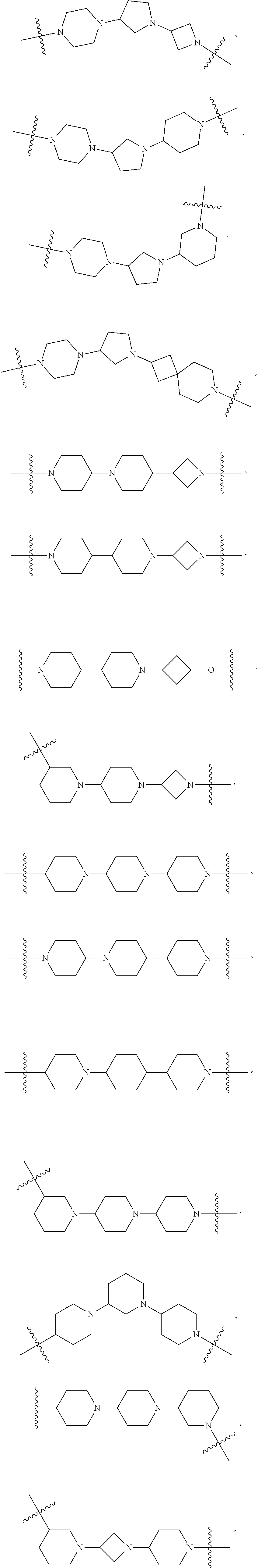 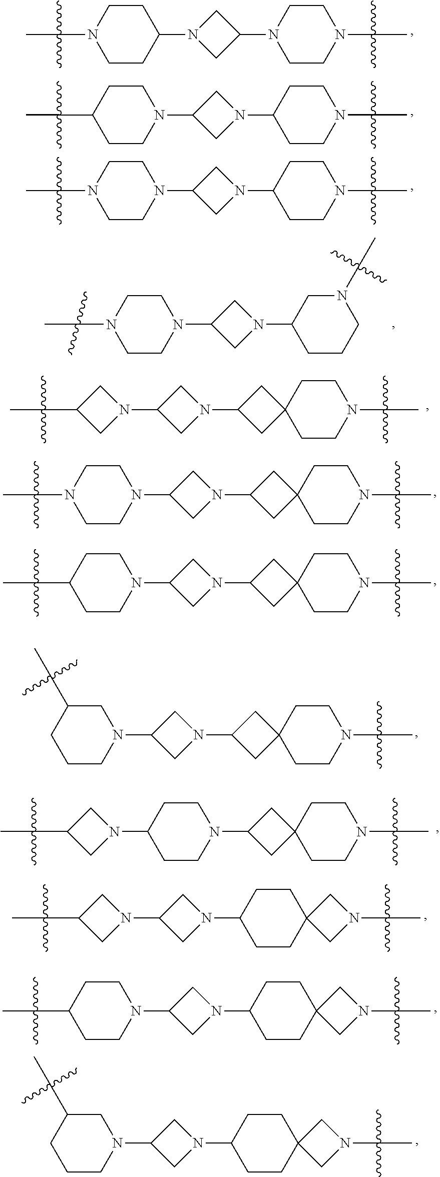 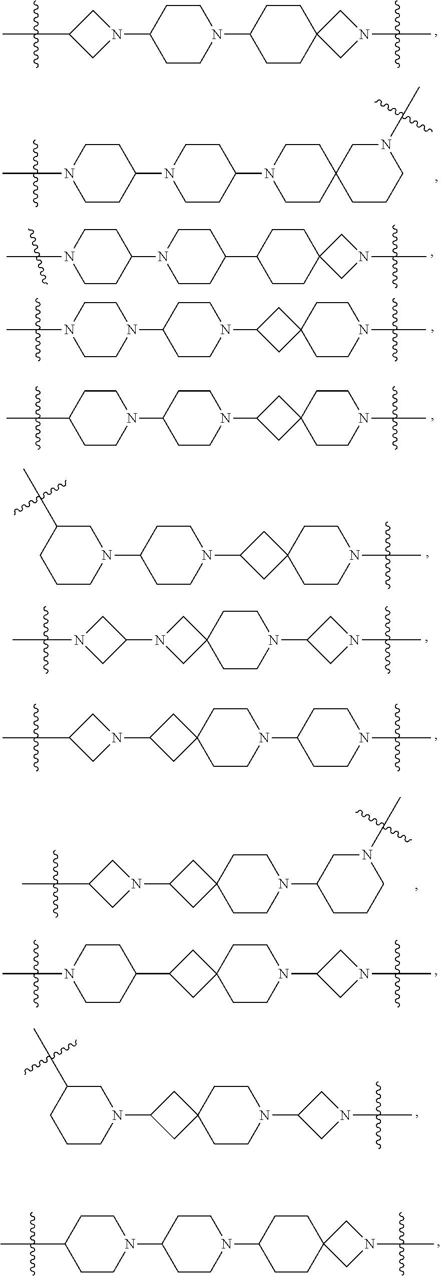 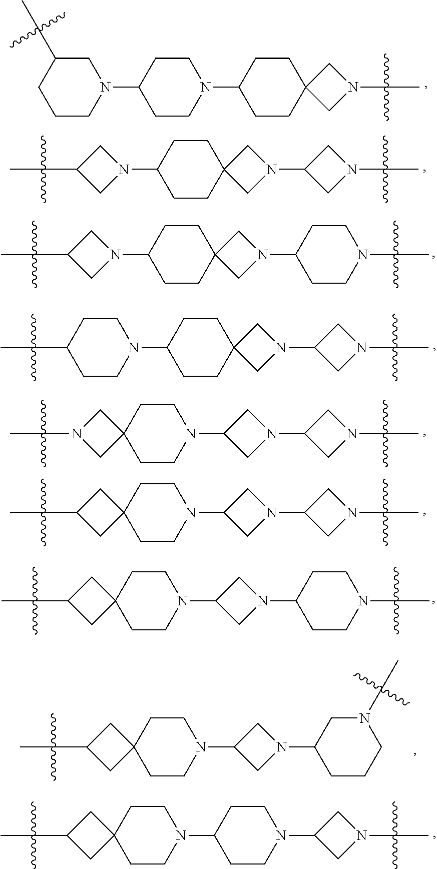 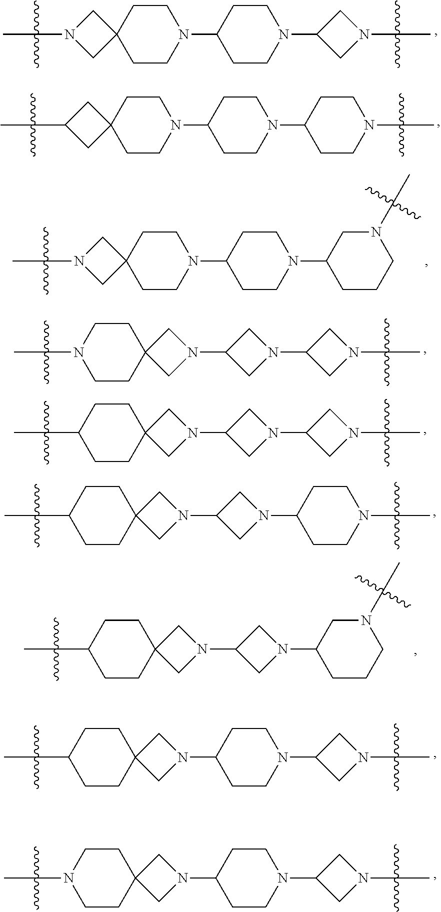 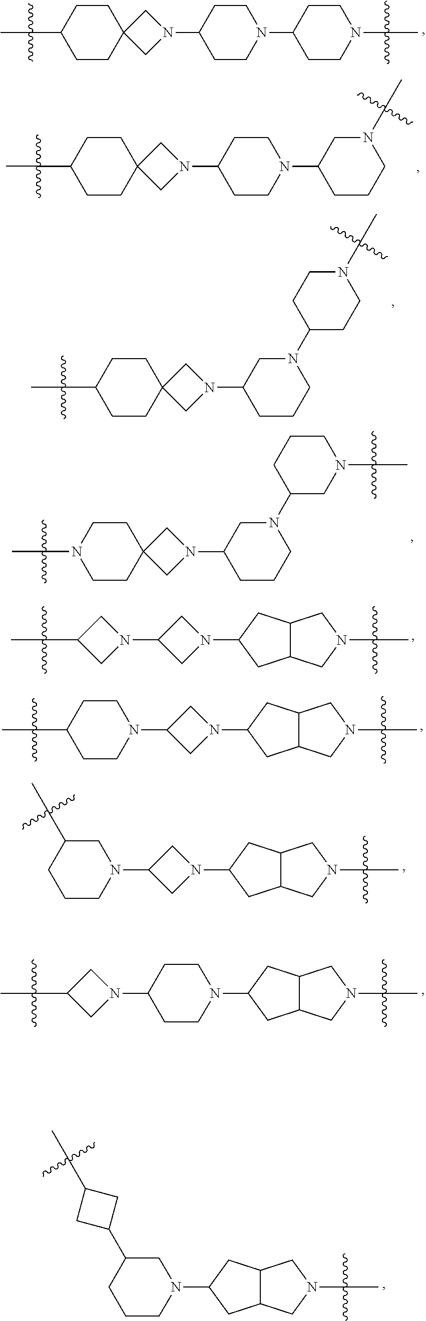 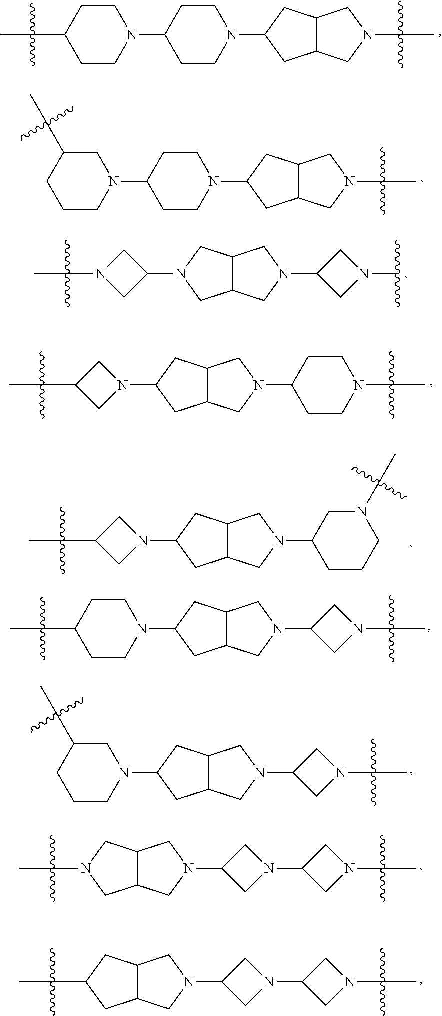 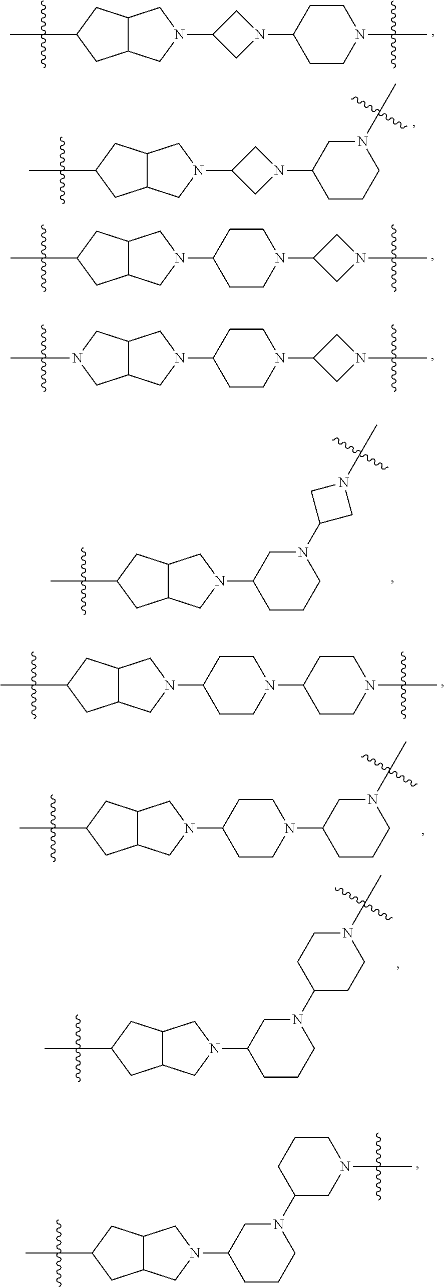 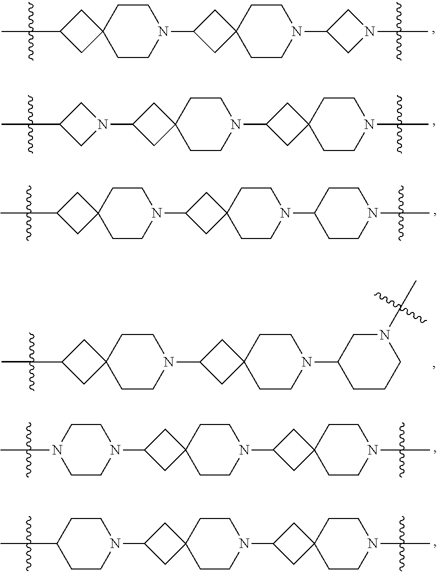 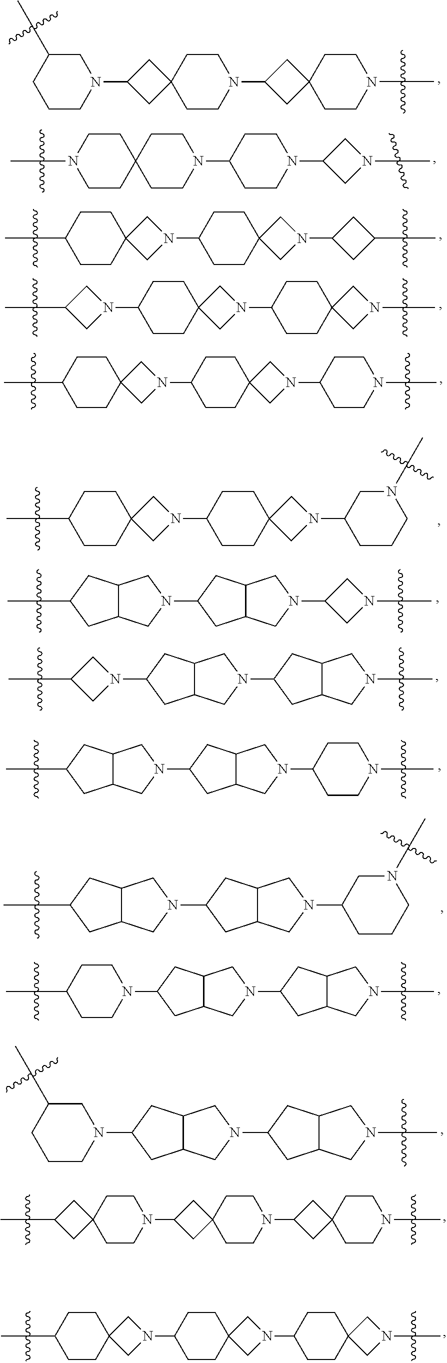 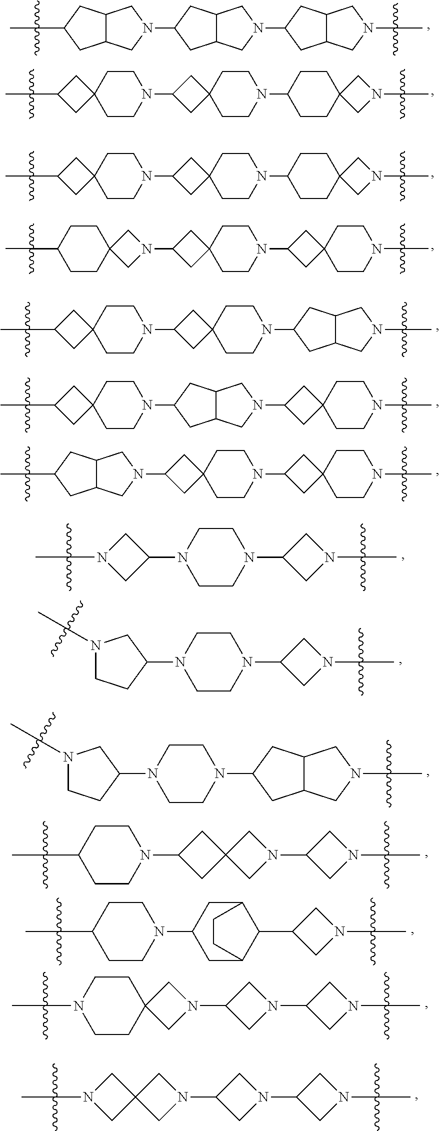 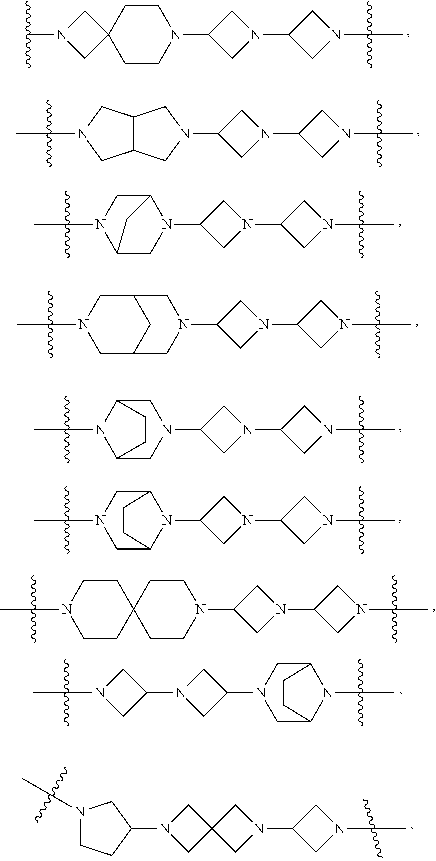 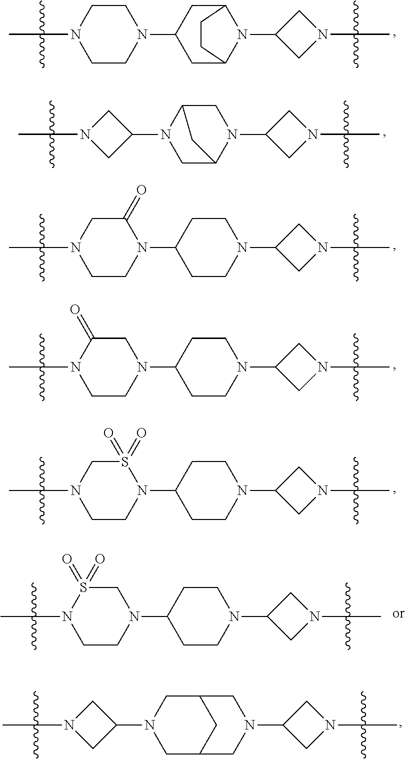
wherein the left side thereof is linked to B;
or L is selected from 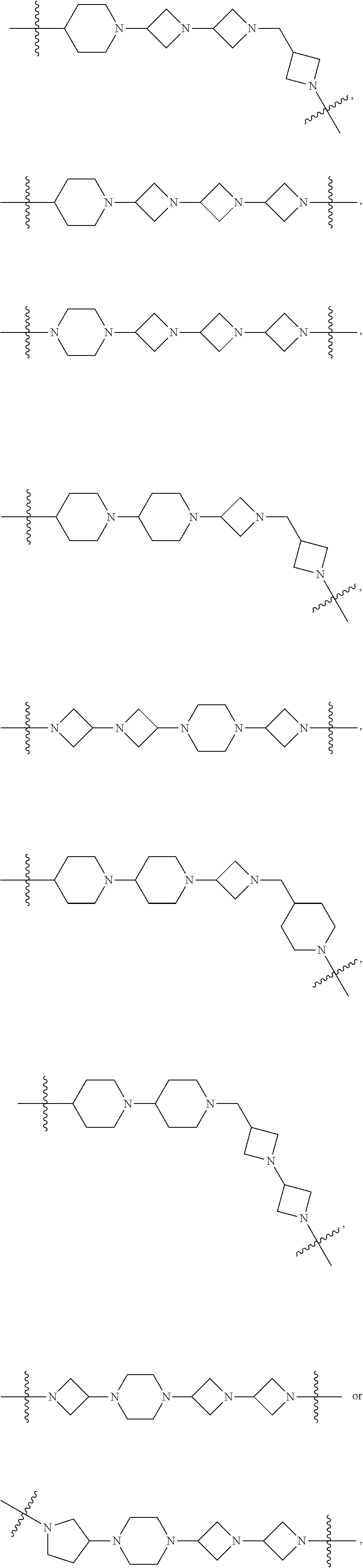
wherein the left side thereof is linked to B;
or L is selected from 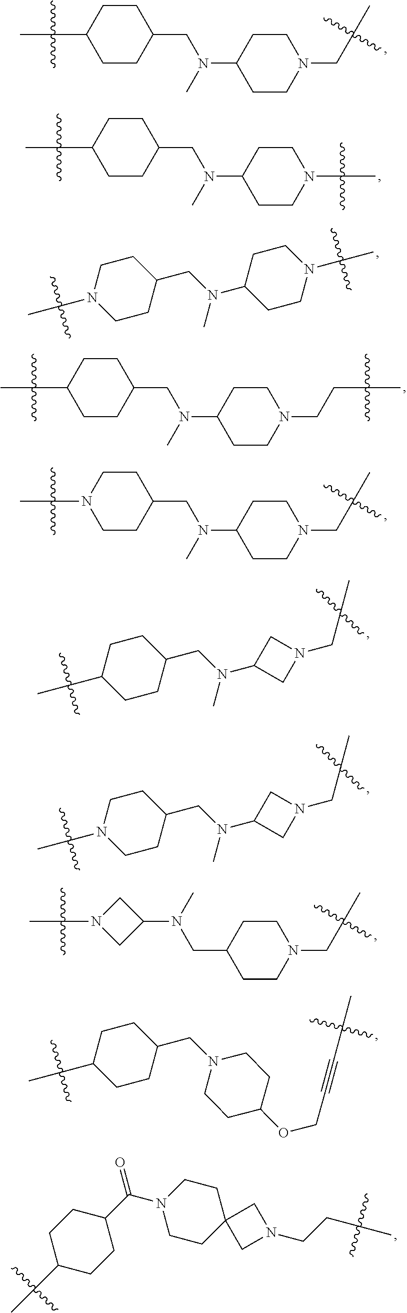 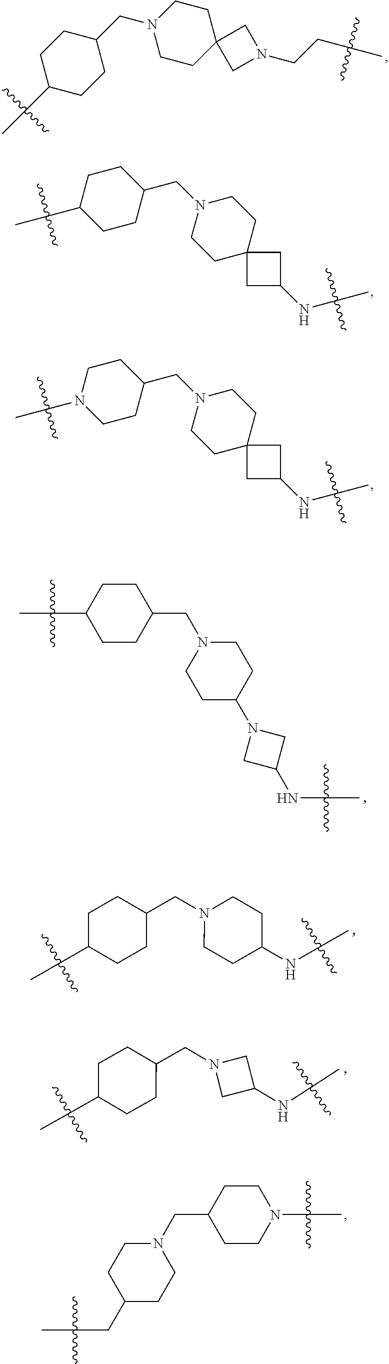 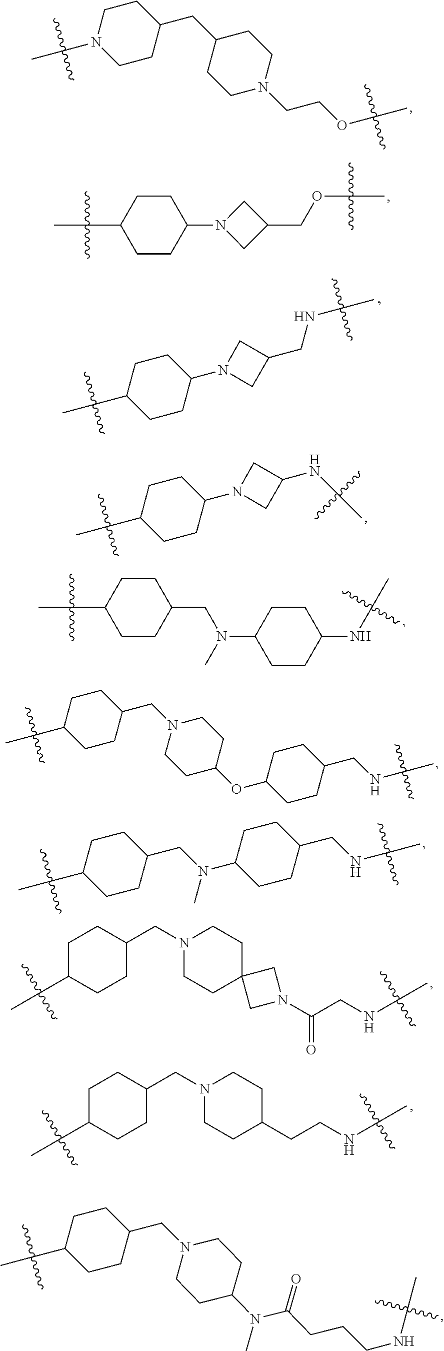 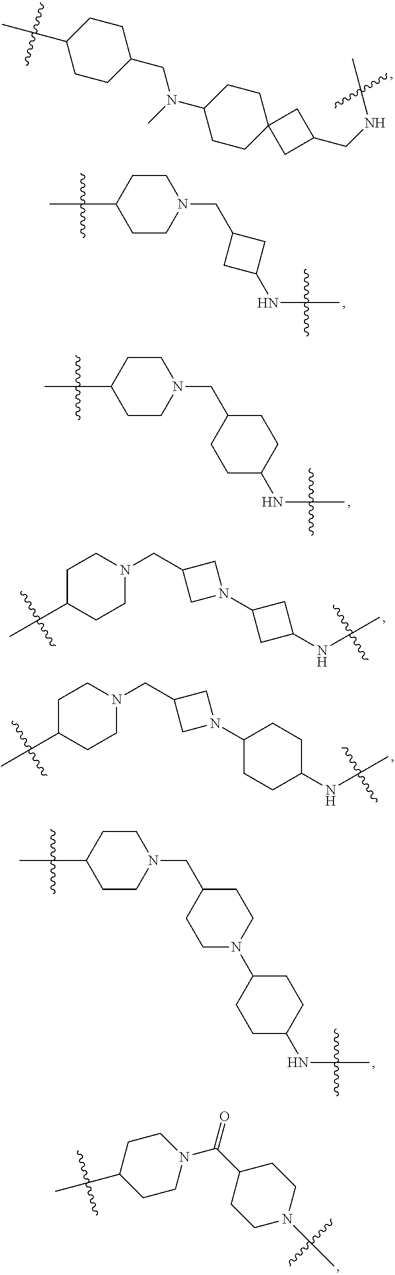 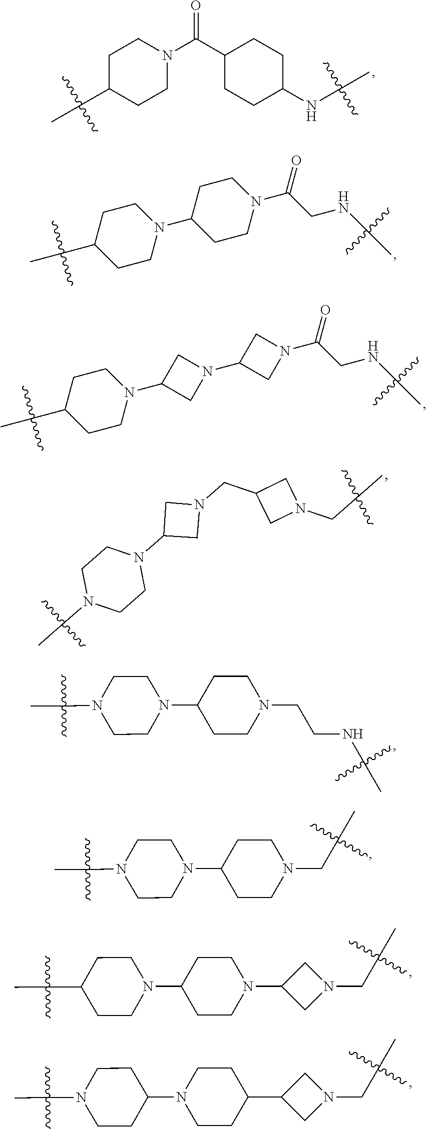 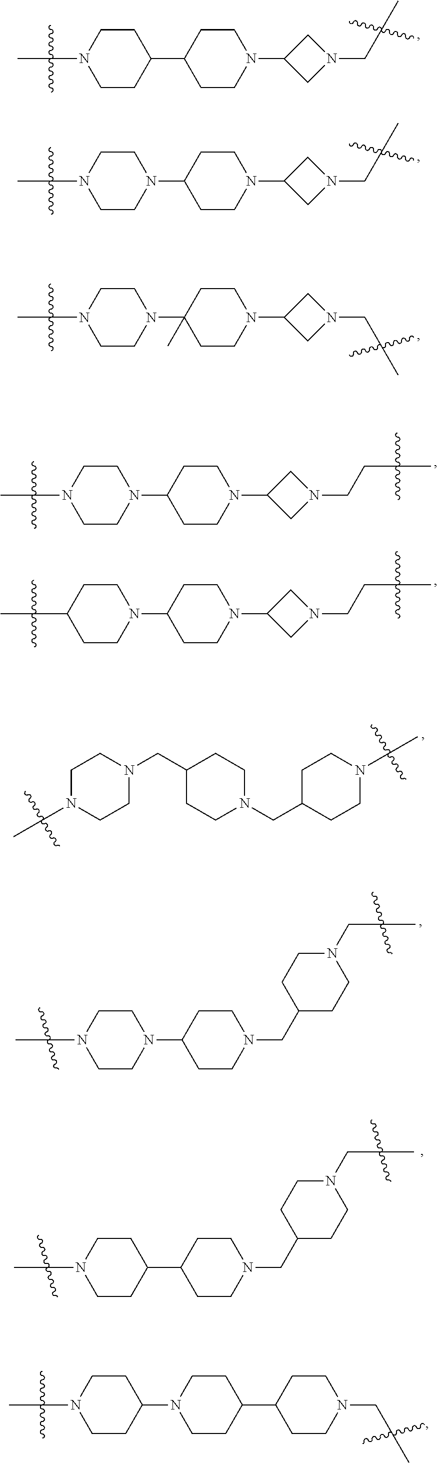 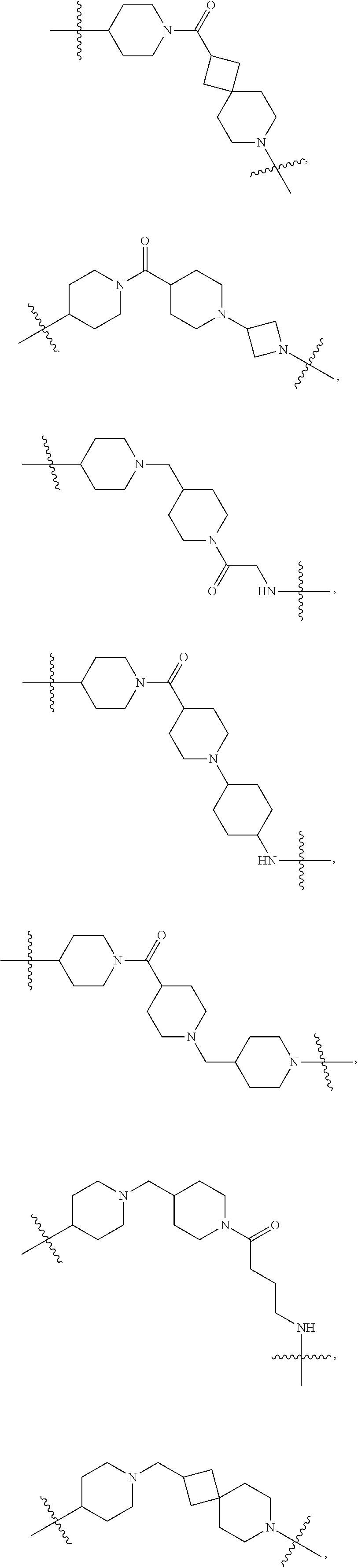 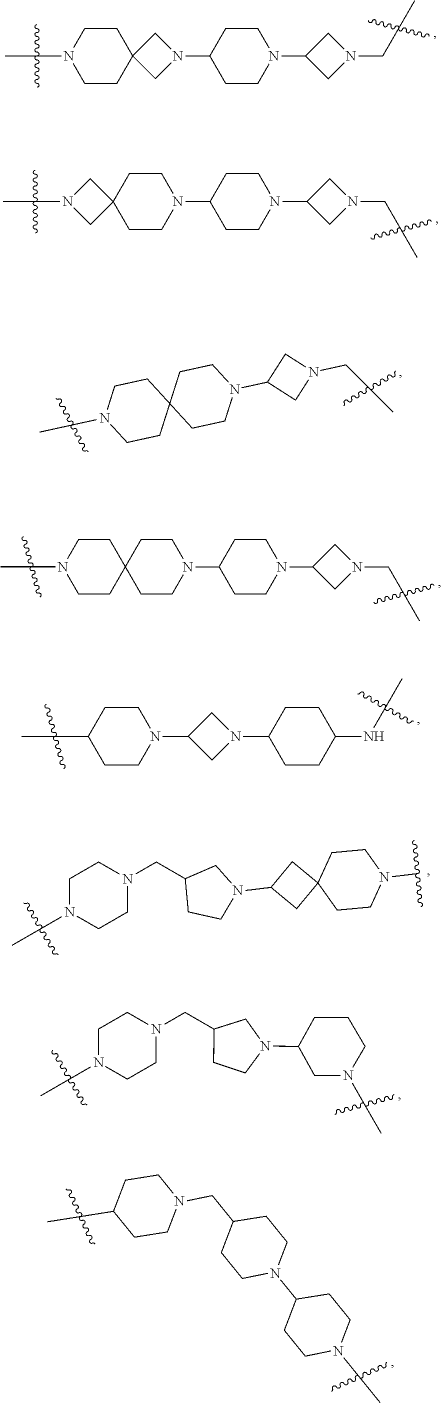 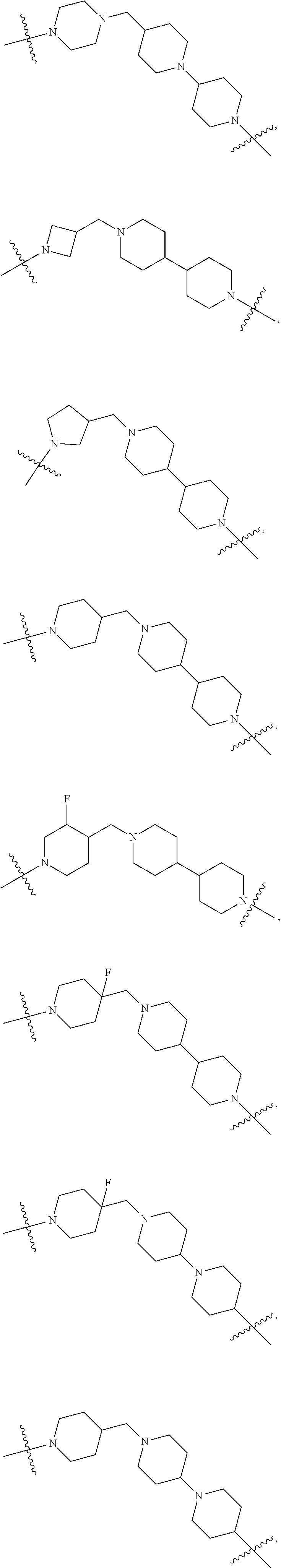 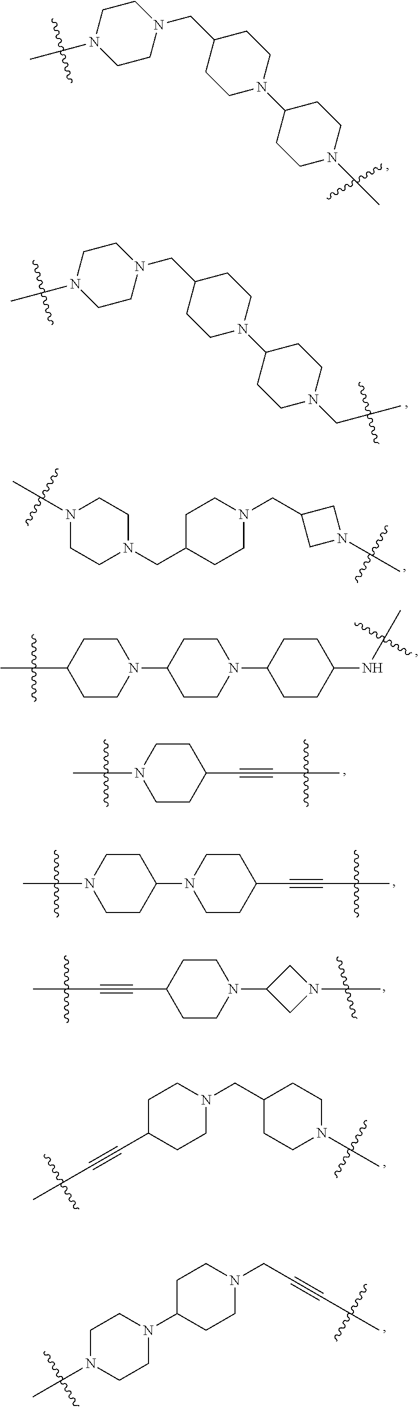 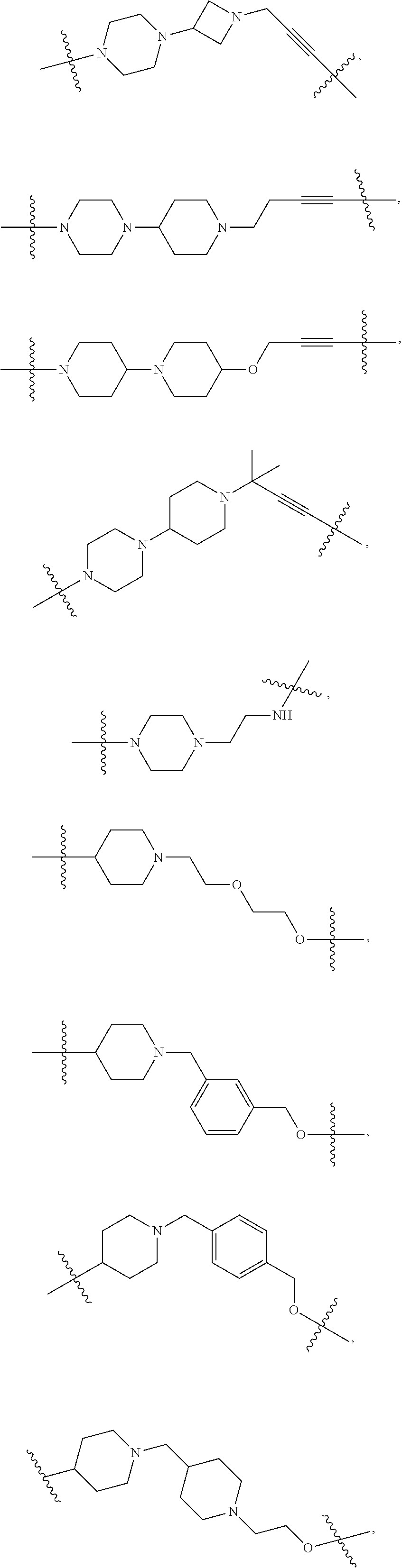 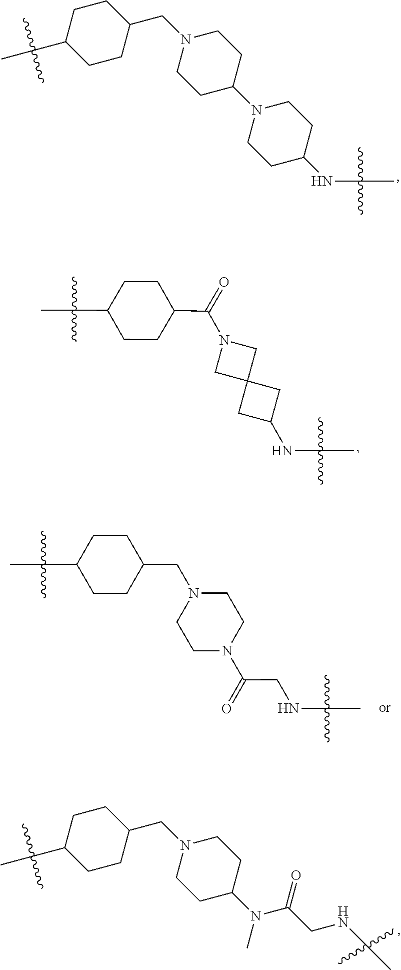 [SMILES: *NC1CCN(C2CCN(CC3CCC(*)CC3)CC2)CC1 ; *NC1CC2(C1)CN(C(=O)C1CCC(*)CC1)C2 ; *NCC(=O)N1CCN(CC2CCC(*)CC2)CC1 ; *NCC(=O)N(C)C1CCN(CC2CCC(*)CC2)CC1]
wherein the left side thereof is linked to B.

7. The compound or the stereoisomer, deuterated compound, solvate, prodrug, metabolite, pharmaceutically acceptable salt or co-crystal thereof according to claim 1, wherein
L is selected from -Cy1-Cy2-Cy3-Cy4-Ak1-Ak2-Ak3-Ak4-Ak5-, -Cy1-Ak1-Cy2-Ak2-Cy3-Ak3-Cy4-Ak4-Ak5-, -Ak1-Cy1-Ak2-Cy2-Ak3-Cy3-Ak4-Cy4-Ak5-, -Cy1-Ak1-Cy2-Ak2-Cy3-Cy4-Ak3-Ak4-Ak5-, -Cy1-Ak1-Cy2-Ak2-Ak3-Cy3-Cy4-Ak4-Ak5-, -Cy1-Ak1-Ak2-Ak3-Ak4-Ak5-Cy2-Cy3-Cy4-, -Cy1-Cy2-Ak1-Ak2-Ak3-Ak4-Ak5-Cy3-Cy4-, -Cy1-Cy2-Cy3-Ak1-Ak2-Ak3-Ak4-Ak5-Cy4-, -Cy1-Cy2-Cy3-Cy4-Ak1-Ak2-Ak3-Ak4-Ak5-, -Cy1-Ak1-Cy2-Cy3-Cy4-Ak2-Ak3-Ak4-Ak5-, -Cy1-Cy2-Ak1-Cy3-Cy4-Ak2-Ak3-Ak4-Ak5-, -Cy1-Cy2-Cy3-Ak1-Cy4-Ak2-Ak3-Ak4-Ak5-, -Cy1-Ak1-Ak2-Cy2-Cy3-Cy4-Ak3-Ak4-Ak5-, -Cy1-Cy2-Ak1-Ak2-Cy3-Cy4-Ak3-Ak4-Ak5-, -Cy1-Cy2-Cy3-Ak1-Ak2-Cy4-Ak3-Ak4-Ak5-, -Cy1-Ak1-Ak2-Ak3-Cy2-Cy3-Cy4-Ak4-Ak5-, -Cy1-Cy2-Ak1-Ak2-Ak3-Cy3-Cy4-Ak4-Ak5-, -Cy1-Cy2-Cy3-Ak1-Ak2-Ak3-Cy4-Ak4-Ak5-, -Cy1-Ak1-Ak2-Ak3-Ak4-Cy2-Cy3-Cy4-Ak5-, -Cy1-Cy2-Ak1-Ak2-Ak3-Ak4-Cy3-Cy4-Ak5-, -Cy1-Cy2-Cy3-Ak1-Ak2-Ak3-Ak4-Cy4-Ak5-, -Ak1-Ak2-Ak3-Ak4-Ak5-Cy1-Cy2-Cy3-Cy4-, -Ak1-Cy1-Cy2-Cy3-Cy4-Ak2-Ak3-Ak4-Ak5-, -Ak1-Ak2-Cy1-Cy2-Cy3-Cy4-Ak3-Ak4-Ak5-, -Ak1-Ak2-Ak3-Cy1-Cy2-Cy3-Cy4-Ak4-Ak5-, -Ak1-Ak2-Ak3-Ak4-Cy1-Cy2-Cy3-Cy4-Ak5-, -Ak1-Cy1-Ak2-Ak3-Ak4-Ak5-Cy2-Cy3-Cy4-, -Ak1-Cy1-Cy2-Ak2-Ak3-Ak4-Ak5-Cy3-Cy4-, -Ak1-Cy1-Cy2-Cy3-Ak2-Ak3-Ak4-Ak5-Cy4-, -Ak1-Ak2-Cy1-Ak3-Ak4-Ak5-Cy2-Cy3-Cy4-, -Ak1-Ak2-Cy1-Cy2-Ak3-Ak4-Ak5-Cy3-Cy4-, -Ak1-Ak2-Cy1-Cy2-Cy3-Ak3-Ak4-Ak5-Cy4-, -Ak1-Ak2-Ak3-Cy1-Ak4-Ak5-Cy2-Cy3-Cy4-, -Ak1-Ak2-Ak3-Cy1-Cy2-Ak4-Ak5-Cy3-Cy4-, -Ak1-Ak2-Ak3-Cy1-Cy2-Cy3-Ak4-Ak5-Cy4-, -Ak1-Ak2-Ak3-Ak4-Cy1-Ak5-Cy2-Cy3-Cy4-, -Ak1-Ak2-Ak3-Ak4-Cy1-Cy2-Ak5-Cy3-Cy4-, or -Ak1-Ak2-Ak3-Ak4-Cy1-Cy2-Cy3-Ak5-Cy4-;
Ak1, Ak2, Ak3, Ak4, and Ak5 are each independently selected from —(CH₂)_{q}—, —(CH₂)_{q}, —O—, —O—(CH₂)_{q}—, —(CH₂)_{q}—NR^{L}—, —NR^{L}—(CH₂)_{q}—, —(CH₂)_{q}—NR^{L}C(═O)—, —(CH₂)_{q}—C(═O)NR^{L}, —C(O)—, —C(═O)—(CH₂)_{q}—NR^{L}, —(C≡C)_{q}— or a bond;
each q is independently selected from 0, 1, 2, 3 or 4;
each R^{L} is independently selected from H or C₁₋₆ alkyl;
the definitions of Cy1, Cy2, Cy3 and Cy4 are the same as those of -Cy- in claim 1.

8. The compound or the stereoisomer, deuterated compound, solvate, prodrug, metabolite, pharmaceutically acceptable salt or co-crystal thereof according to claim 7, wherein
Cy1, Cy2, Cy3 and C₄ are each independently selected from a bond, 4- to 7-membered nitrogen-containing mono-heterocyclic ring, 4- to 10-membered nitrogen-containing fused heterocyclic ring, 5- to 12-membered nitrogen-containing spiro-heterocyclic ring, 7- to 10-membered nitrogen-containing bridged-heterocyclic ring, 3- to 7-membered monocycloalkyl, 4- to 10-membered fused cycloalkyl, 5- to 12-membered spiro cycloalkyl, 7- to 10-membered bridged cycloalkyl, 5- to 10-membered heteroaryl or 6- to 10-membered aryl, wherein the mono-heterocyclic ring, fused heterocyclic ring, bridged-heterocyclic ring, spiro-heterocyclic ring, cycloalkyl, aryl or heteroaryl is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring, bridged-heterocyclic ring, spiro-heterocyclic ring or heteroaryl contains 1 to 4 heteroatoms selected from O, S, or N;
each R^{L} is independently selected from H or C₁₋₄ alkyl;
each R^{w1} is independently selected from H, methyl, ethyl, isopropyl, cyclopropyl or cyclobutyl;
each R^{w2} or R^{w3} is independently selected from H, F, Cl, Br, I, OH, NH₂, CN, COOH, CONH₂, methyl, ethyl, isopropyl, ethenyl, ethynyl, methoxy, ethoxy, propoxy, cyclopropyl or cyclobutyl, wherein the methyl, ethyl, isopropyl, ethenyl, ethynyl, methoxy, ethoxy, propoxy, cyclopropyl or cyclobutyl is optionally further substituted with 0 to 4 substituents selected from H, F, Cl, Br, I, OH, or NH₂;
alternatively, R^{w2} and R^{w3} together with the carbon atoms to which they are attached form cyclopropyl, cyclobutyl, cyclopentyl, azetidinyl, oxetanyl, pyrrolidinyl, or piperidyl, wherein the cyclopropyl, cyclobutyl, cyclopentyl, azetidinyl, oxetanyl, pyrrolidinyl, or piperidyl is optionally further substituted with 0 to 4 substituents selected from H, F, Cl, Br, I, OH, NH₂, CN, CF₃, or CHF₂;
each R^{b1} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, deuterated methoxy, methylthio, ethylthio, morpholinyl, piperazinyl, pyrrolidinyl, piperidyl or oxazolidinyl, wherein the methoxy, ethoxy, propoxy, isopropyloxy, methylthio, or ethylthio is optionally further substituted with 0 to 4 substituents selected from H, F, Cl, Br, I, OH, or NH₂;
each R^{b2} is independently selected from H, F, Cl, Br, I, ═O, OH, NH₂, NO₂, COOH, NHCH₃, NHCH₂CH₃, NHCH(CH₃)₂, N(CH₃)₂, N(CH₂CH₃)₂, CN, CF₃, CHF₂, methyl, ethyl, ethenyl, ethynyl, methoxy, ethoxy, propoxy, isopropyloxy, morpholinyl, piperazinyl, pyrrolidinyl, piperidyl or oxazolidinyl, wherein the methyl, ethyl, ethenyl, ethynyl, methoxy, ethoxy, propoxy, isopropyloxy, morpholinyl, piperazinyl, pyrrolidinyl, piperidyl or oxazolidinyl is optionally further substituted with 0 to 4 substituents selected from H, F, Cl, Br, I, OH, or NH₂;
each R^{b3} is independently selected from H, F, Cl, Br, I, ═O, CF₃, CHF₂, OH, CN, COOH, NH₂, CONH₂, S(═O)₂NH₂, S(═O)₂CH₃, methyl, ethyl, isopropyl, ethenyl, ethynyl, methoxy, ethoxy, propoxy, methylthio or ethylthio, wherein the methyl, ethyl, isopropyl, ethenyl, ethynyl, methoxy, ethoxy, propoxy, methylthio or ethylthio is optionally further substituted with 0 to 4 substituents selected from H, F, Cl, Br, I, OH, or NH₂;
r is selected from 1 or 2;
each t is independently selected from 0, 1 or 2;
K is selected from 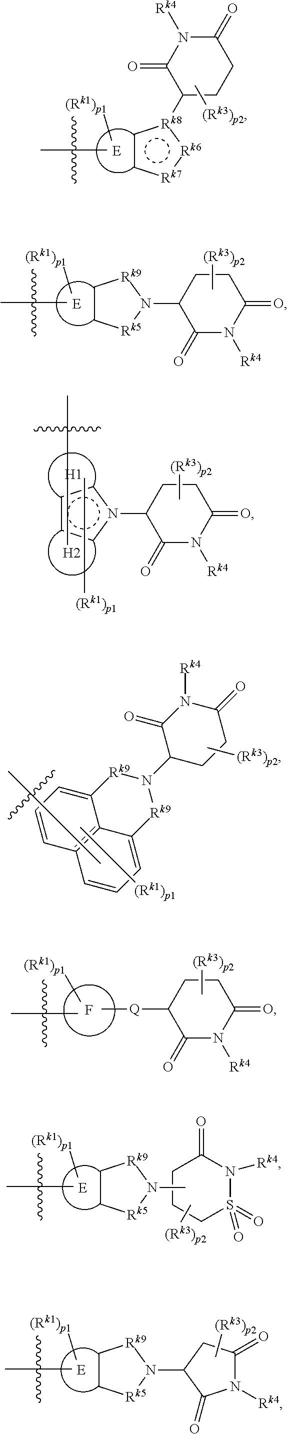 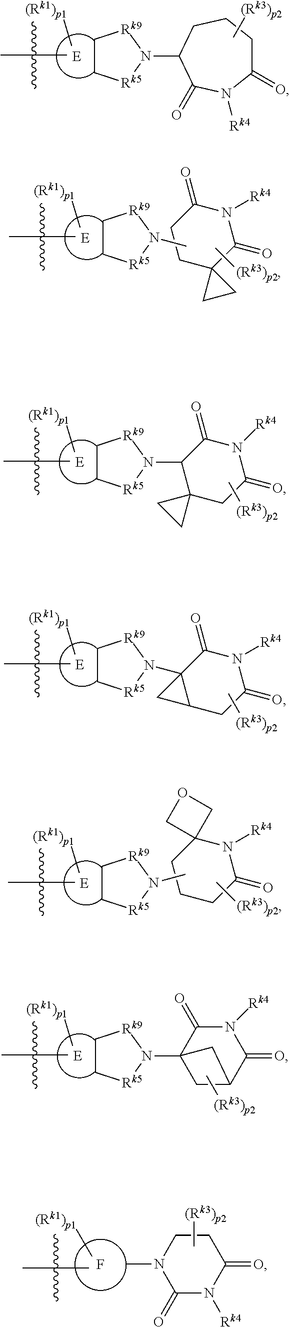 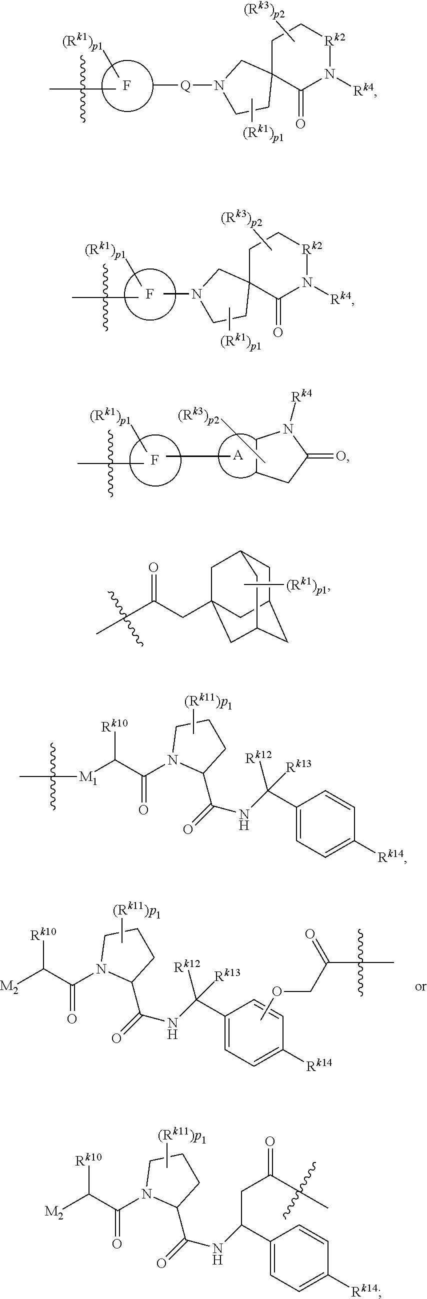
or K is selected from 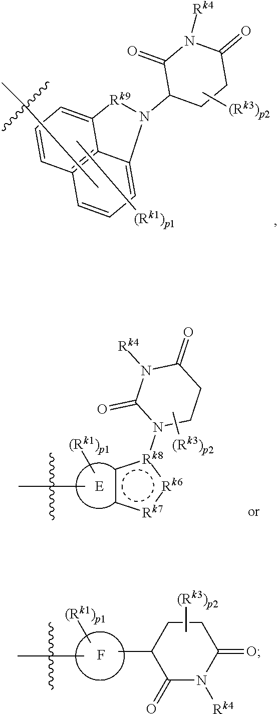 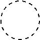
represents a ring selected from an aromatic ring or a non-aromatic ring;
each Q is independently selected from —O—, —S—, —CH₂—, —NR^{q}—, —CO—, —NR^{q}CO—, —CONR^{q}— or 4- to 7-membered heterocyclyl, wherein the heterocyclyl is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocyclyl contains 1 to 4 heteroatoms selected from O, S, or N;
R^{q} is selected from H or C₁₋₄ alkyl;
R^{k1} and R^{k3} are each independently selected from H, F, Cl, Br, I, OH, ═O, NH₂, CF₃, CN, COOH, CONH₂, C₁₋₄ alkyl or C₁₋₄ alkoxy, wherein the alkyl or alkoxy is optionally further substituted with 0 to 4 substituents selected from H, F, Cl, Br, I, OH, or NH₂;
or two R^{k3} together with the carbon atoms or ring backbones to which they are directly attached form 3- to 6-membered carbocycle or 3- to 7-membered heterocycle, wherein the carbocycle or heterocycle is optionally further substituted with 0 to 4 substituents selected from H, F, Cl, Br, I, OH, ═O, NH₂, CN, COOH, CONH₂, C₁₋₄ alkyl or C₁₋₄ alkoxy, and the heterocycle contains 1 to 4 heteroatoms selected from O, S, or N;
each R^{k4} is independently selected from H, OH, NH₂, CF₃, CN, or C₁₋₄ alkyl;
each R^{k5} is independently selected from CO, CH₂, SO₂ or 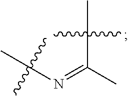 [SMILES: */N=C(\*)C]
each R^{k6} is independently selected from CO, CH, SO, SO₂, CH₂ or N;
each R^{k7} is independently selected from CO, CH, N, CH₂, O, S, N(CH₃) or NH;
each R^{k8} is independently selected from C, N or CH;
each R^{k9} is independently selected from CO, CH₂ or SO₂;
each A, H1 or H2 is independently selected from C₃₋₈ carbocycle, a benzene ring, 4- to 7-membered heterocycle or 5- to 6-membered heteroaryl, wherein the heterocycle or heteroaryl contains 1 to 4 heteroatoms selected from O, S, or N;
each E is independently selected from C₃₋₈ carbocycle, a benzene ring, 4- to 7-membered heterocycle, 8- to 12-membered heterocycle, 7- to 12-membered heteroaryl or 5- to 6-membered heteroaryl, wherein the heterocycle or heteroaryl contains 1 to 4 heteroatoms selected from O, S, or N;
each F is independently selected from 3- to 7-membered monocycloalkyl, 4- to 10-membered fused cycloalkyl, 5- to 12-membered spiro cycloalkyl, 5- to 10-membered bridged cycloalkyl, 4- to 7-membered mono-heterocyclic ring, 4- to 10-membered fused heterocyclic ring, 5- to 12-membered spiro-heterocyclic ring, 5- to 10-membered bridged-heterocyclic ring, C₆₋₁₄ aryl or 5- to 10-membered heteroaryl, wherein the mono-heterocyclic ring, fused heterocyclic ring, spiro-heterocyclic ring, bridged-heterocyclic ring or heteroaryl contains 1 to 4 heteroatoms selected from O, S, or N.

9. The compound or the stereoisomer, deuterated compound, solvate, prodrug, metabolite, pharmaceutically acceptable salt or co-crystal thereof according to claim 8, wherein
R^{L} is selected from H, methyl or ethyl;
each q is independently selected from 0, 1 or 2;
Cy1, Cy2, Cy3 and Cy4 are each independently selected from a bond or one of the following substituted or unsubstituted groups: cyclopropyl, cyclobutyl, cyclopentyl, cyclohexyl, azetidinyl, azacyclopentyl, piperidine, azacyclohexenyl, morpholine, piperazine, phenyl, cyclopropyl-fused-cyclopropyl, cyclopropyl-fused-cyclobutyl, cyclopropyl-fused-cyclopentyl, cyclopropyl-fused-cyclohexyl, cyclobutyl-fused-cyclobutyl, cyclobutyl-fused-cyclopentyl, cyclobutyl-fused-cyclohexyl, cyclopentyl-fused-cyclopentyl, cyclopentyl-fused-cyclohexyl, cyclohexyl-fused-cyclohexyl, cyclopropyl-spiro-cyclopropyl, cyclopropyl-spiro-cyclobutyl, cyclopropyl-spiro-cyclopentyl, cyclopropyl-spiro-cyclohexyl, cyclobutyl-spiro-cyclobutyl, cyclobutyl-spiro-cyclopentyl, cyclobutyl-spiro-cyclohexyl, cyclopentyl-spiro-cyclopentyl, cyclopentyl-spiro-cyclohexyl, cyclohexyl-spiro-cyclohexyl, cyclopropyl-fused-azetidinyl, cyclopropyl-fused-azacyclopentyl, cyclopropyl-fused-azacyclohexyl, cyclopropyl-fused-piperidine, cyclobutyl-fused-azetidinyl, cyclobutyl-fused-azacyclopentyl, cyclobutyl-fused-azacyclohexyl, cyclobutyl-fused-piperidine, cyclopentyl-fused-azetidinyl, cyclopentyl-fused-azacyclopentyl, cyclopentyl-fused-azacyclohexyl, cyclopentyl-fused-piperidine, cyclohexyl-fused-azetidinyl, cyclohexyl-fused-azacyclopentyl, cyclohexyl-fused-azacyclohexyl, cyclohexyl-fused-piperidine, azetidinyl-fused-azetidinyl, azetidinyl-fused-azacyclopentyl, azetidinyl-fused-azacyclohexyl, azetidinyl-fused-piperidine, azacyclopentyl-fused-azetidinyl, azacyclopentyl-fused-azacyclopentyl, azacyclopentyl-fused-azacyclohexyl, azacyclopentyl-fused-piperidine, azacyclohexyl-fused-azetidinyl, azacyclohexyl-fused-azacyclopentyl, azacyclohexyl-fused-azacyclohexyl, azacyclohexyl-fused-piperidine, cyclobutyl-spiro-azetidinyl, cyclobutyl-spiro-azacyclopentyl, cyclobutyl-spiro-azacyclohexyl, cyclopentyl-spiro-azetidinyl, cyclopentyl-spiro-azacyclopentyl, cyclopentyl-spiro-azacyclohexyl, cyclohexyl-spiro-azetidinyl, cyclohexyl-spiro-azacyclopentyl, cyclohexyl-spiro-azacyclohexyl, azetidinyl-spiro-azetidinyl, azetidinyl-spiro-azacyclopentyl, azetidinyl-spiro-azacyclohexyl, azacyclopentyl-spiro-azetidinyl, azacyclopentyl-spiro-azacyclopentyl, azacyclopentyl-spiro-azacyclohexyl, azacyclohexyl-spiro-azetidinyl, azacyclohexyl-spiro-azacyclopentyl, azacyclohexyl-spiro-azacyclohexyl, cyclobutyl-spiro-piperidine, cyclopentyl-spiro-piperidine, cyclohexyl-spiro-piperidine, azetidinyl-spiro-piperidine, azacyclopentyl-spiro-piperidine, azacyclohexyl-spiro-piperidine, 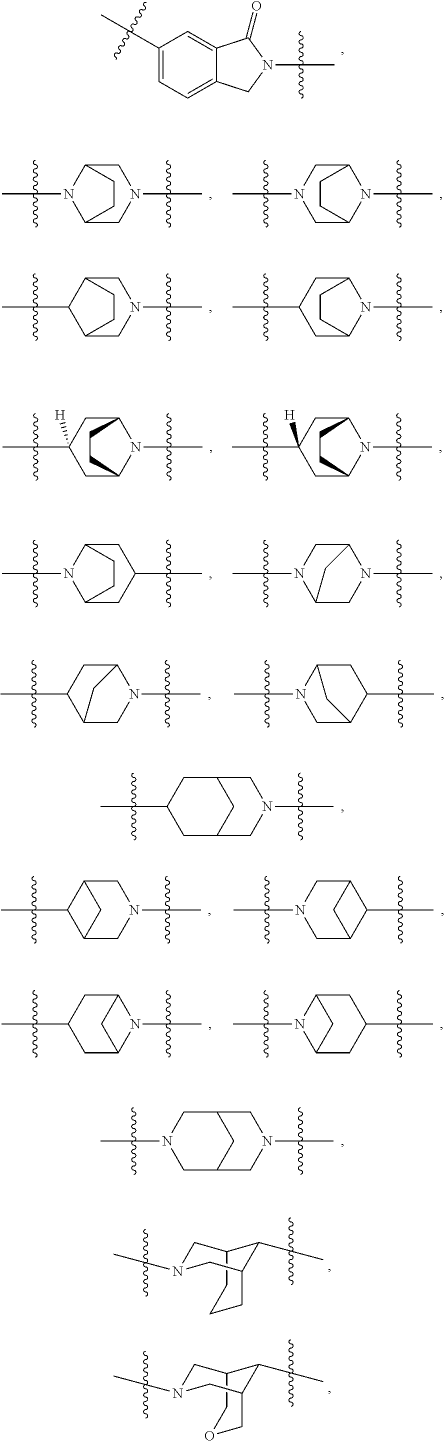 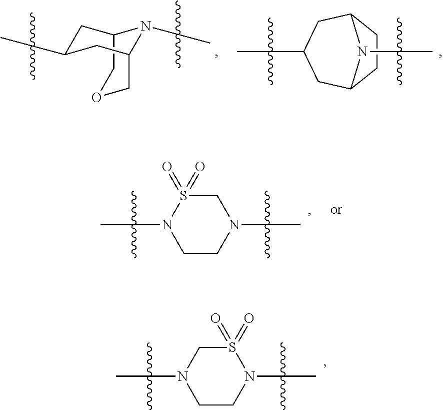 [SMILES: *C1CC2COCC(C1)N2* ; *C1CC2CCC(C1)N2* ; *N1CCN(*)S(=O)(=O)C1 ; *N1CCN(*)S(=O)(=O)C1]
which, when substituted, is optionally further substituted with 0 to 4 substituents selected from H, F, Cl, Br, I, OH, NH₂, COOH, CN, ═O, C₁₋₄ alkyl, halogen-substituted C₁₋₄ alkyl, hydroxyl-substituted C₁₋₄ alkyl or C₁₋₄ alkoxy;
B is selected from 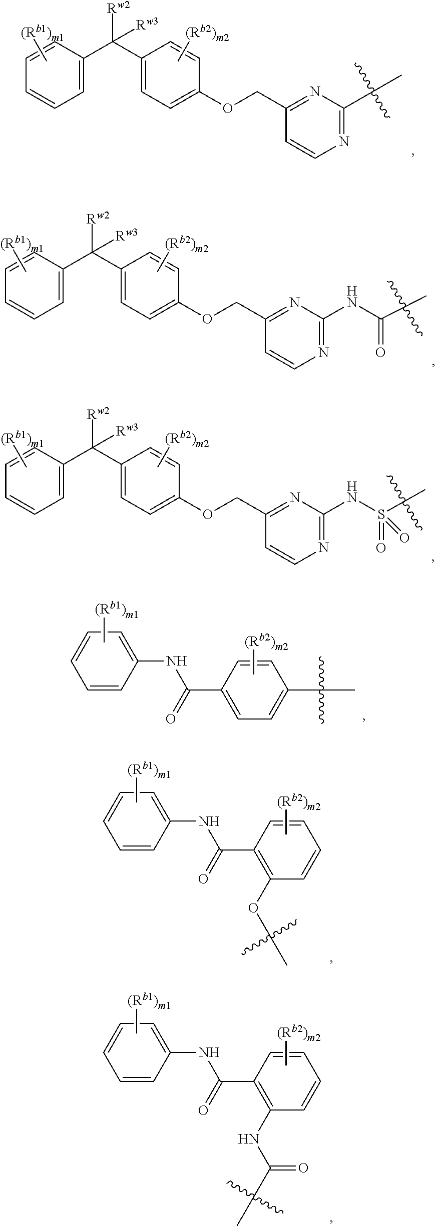 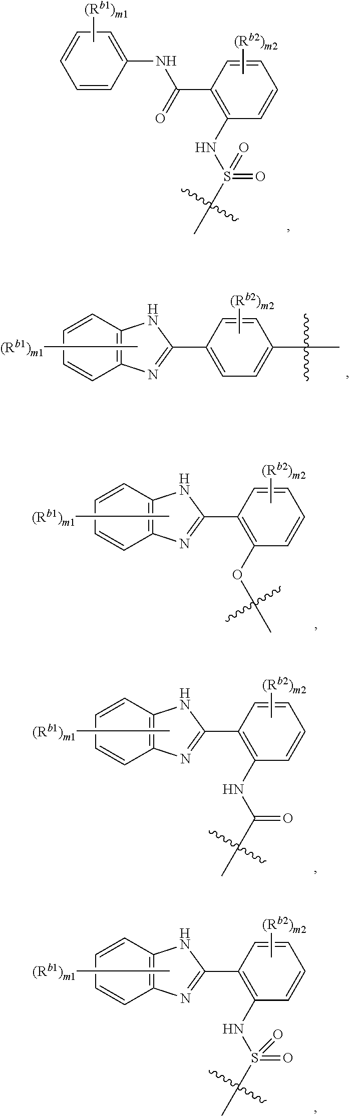 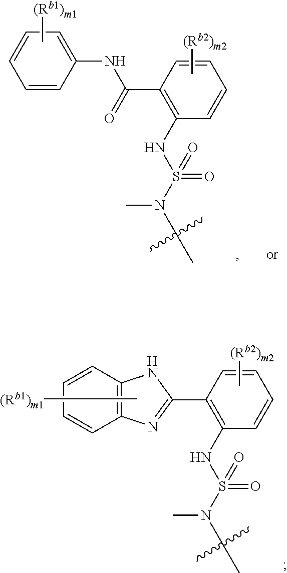
or B is selected from 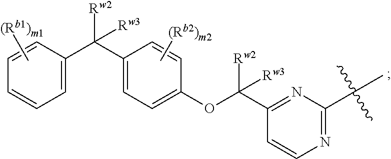
K is selected from 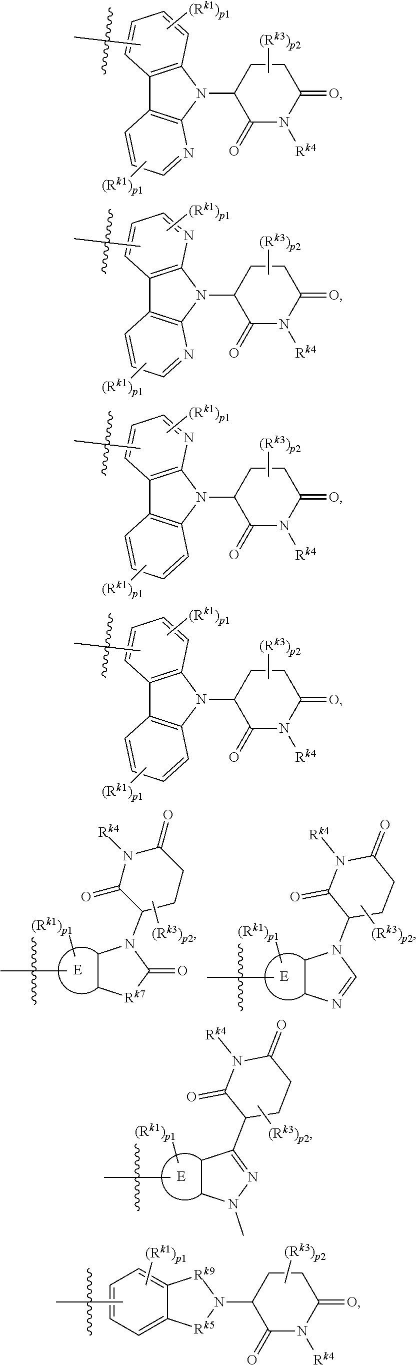 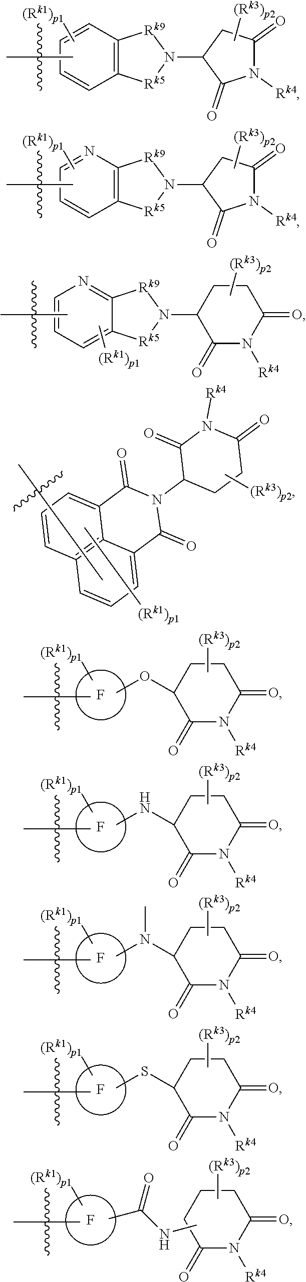 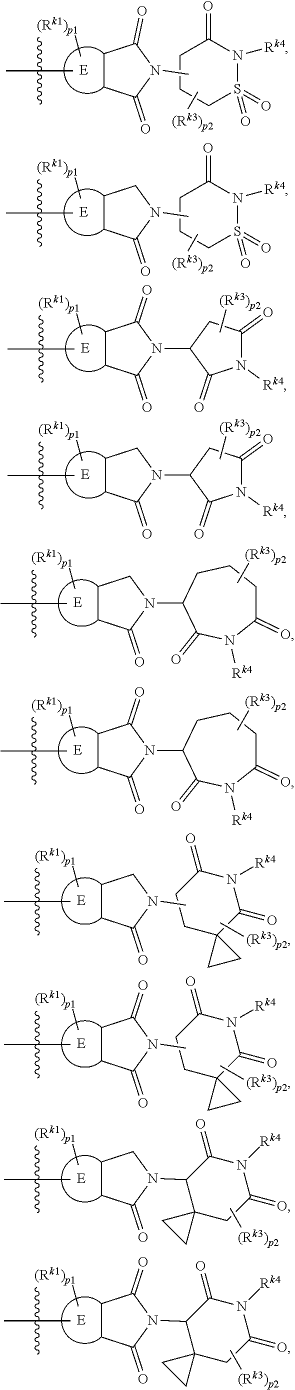 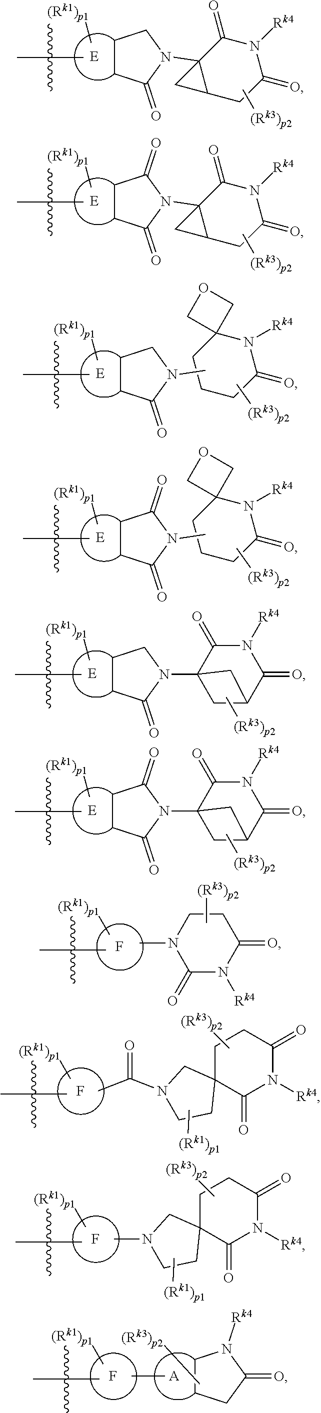 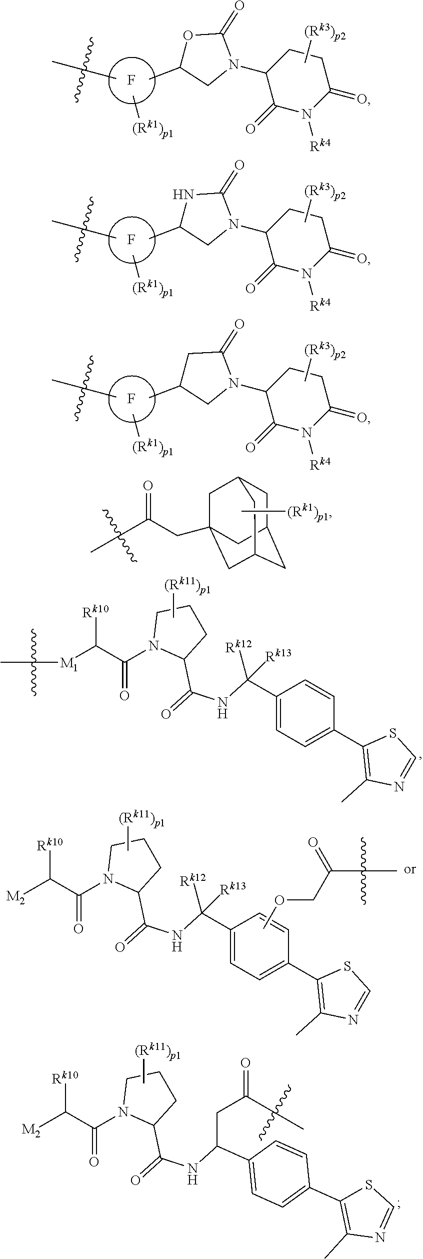
or K is selected from 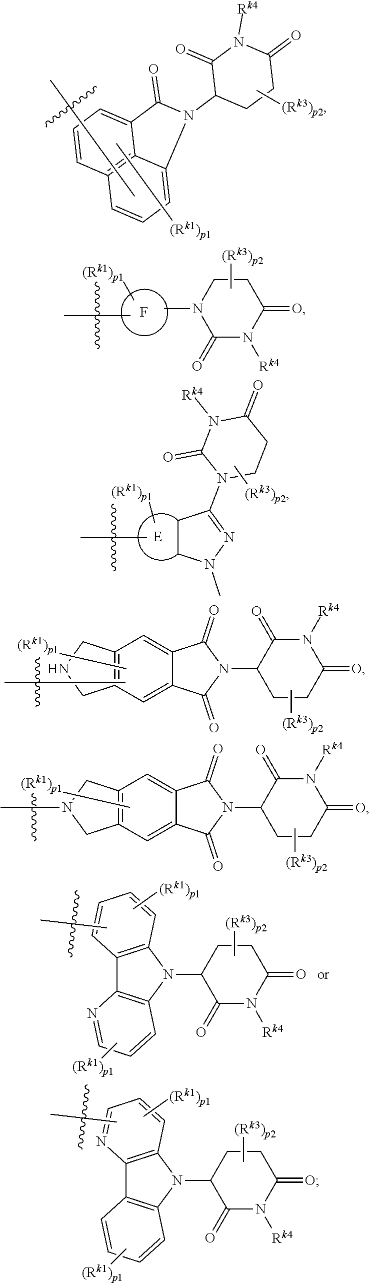
each E is independently selected from phenyl, pyridyl, pyridazinyl, pyrazinyl, pyrimidinyl, pyrrolyl, pyrazolyl, imidazoly, thiazolyl, furyl, thienyl or oxazolyl;
each A is independently selected from phenyl, pyridyl, pyridazinyl, pyrazinyl, pyrimidinyl, pyrrolyl, pyrazolyl, imidazoly, thiazolyl, furyl, thienyl or oxazolyl;
each F is independently selected from cyclopropyl, cyclobutyl, cyclopentyl, cyclohexyl, phenyl, naphthyl, anthryl, phenanthrenyl, azetidinyl, azacyclopentyl, piperidyl, morpholinyl, pyridyl, pyrimidinyl, pyridazinyl, pyrazinyl, triazinyl, pyrrolyl, pyrazolyl, imidazolyl, triazolyl, oxazolyl, furyl, thienyl, thiazolyl, benzoimidazolyl, benzopyrazolyl, benzothiazolyl, benzothienyl, benzofuryl, benzopyrrolyl, benzopyridyl, benzopyrazinyl, benzopyrimidinyl, benzopyridazinyl, pyrrolopyrrolyl, pyrrolopyridinyl, pyrrolopyrimidinyl, pyrrolopyridazinyl, pyrrolopyrazinyl, imidazopyrimidinyl, imidazopyridinyl, imidazopyrazinyl, imidazopyridazinyl, pyrazolopyridinyl, pyrazolopyrimidinyl, pyrazolopyridazinyl, pyrazolopyrazinyl, pyrimidopyridyl, pyrimidopyrazinyl, pyrimidopyridazinyl, pyrimidopyrimidinyl, pyridopyridyl, pyridopyrazinyl, pyridopyridazinyl, pyridazopyridazinyl, pyridazopyrazinyl or pyrazinopyrazinyl;
each R^{k7} is independently selected from CH₂, O, N(CH₃) or NH;
each p1 or p2 is independently selected from 0, 1 or 2.

10. The compound or the stereoisomer, deuterated compound, solvate, prodrug, metabolite, pharmaceutically acceptable salt or co-crystal thereof according to claim 9, wherein
Cy1, Cy2, Cy3 and Cy4 are each independently selected from a bond or one of the following substituted or unsubstituted groups: 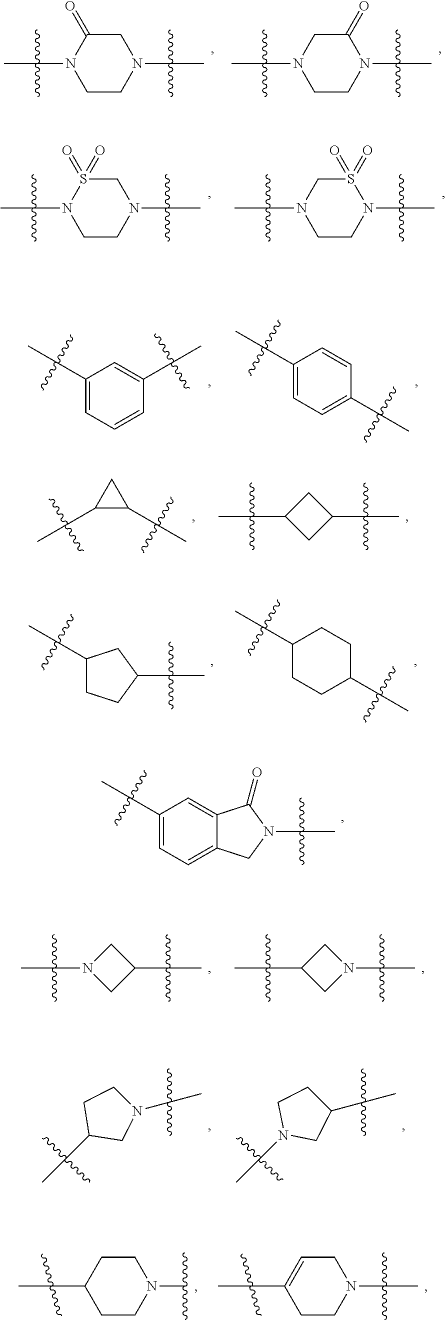 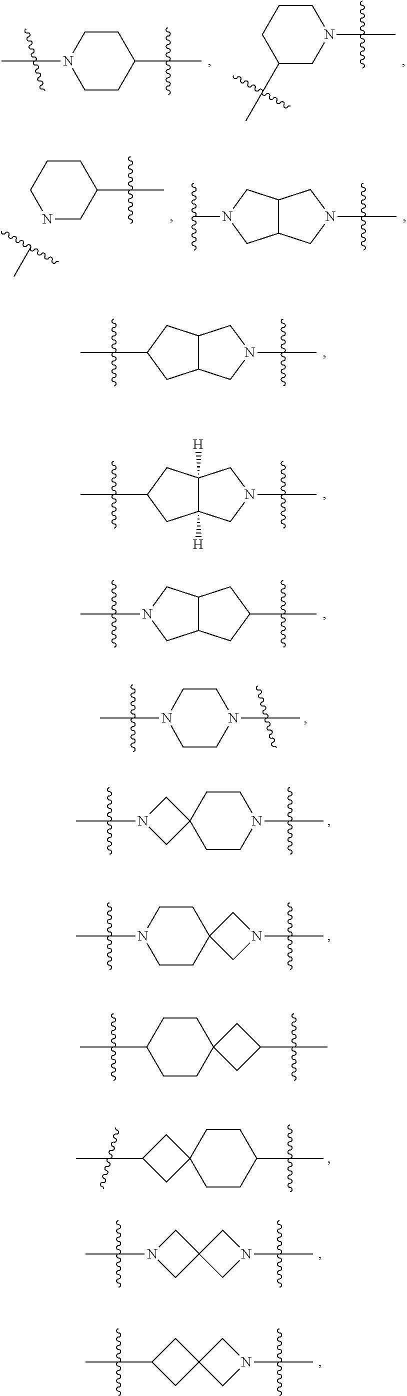 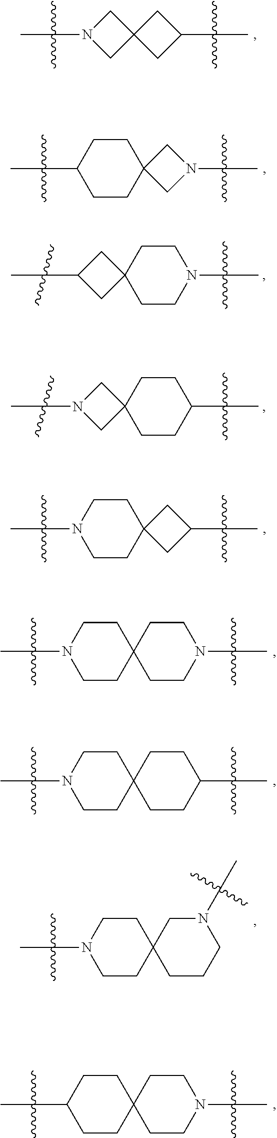 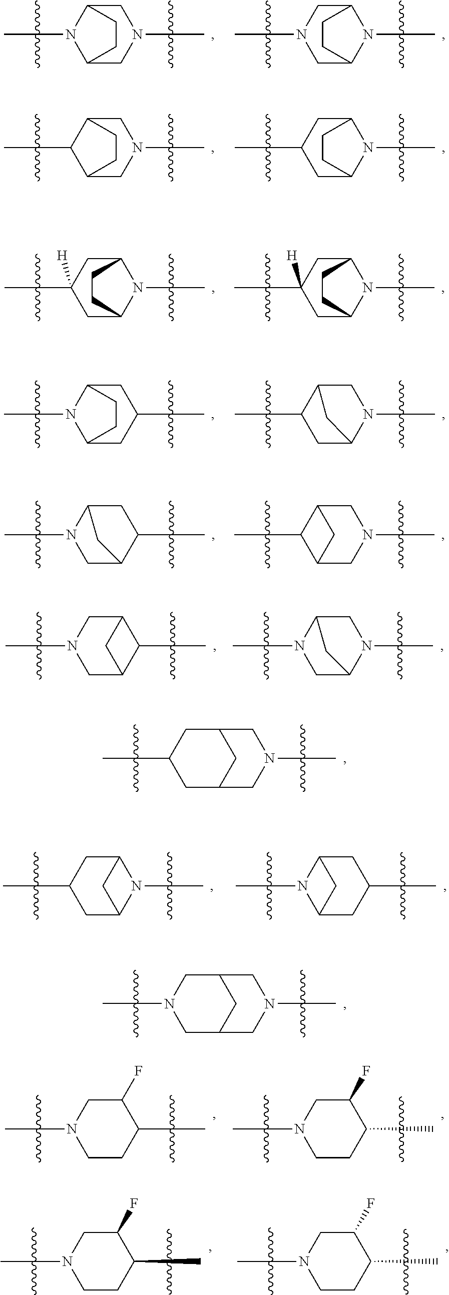 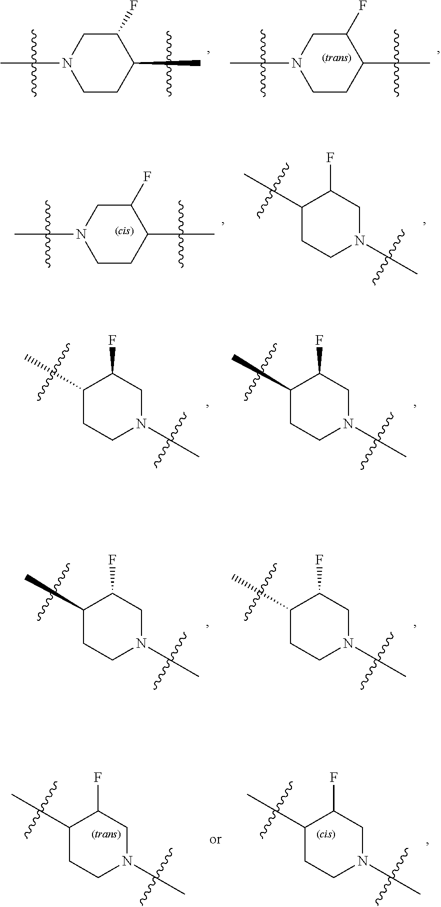
which, when substituted, is optionally further substituted with 0 to 4 substituents selected from H, F, CF₃, methyl, ═O, hydroxymethyl, COOH, CN or NH₂;
K is selected from 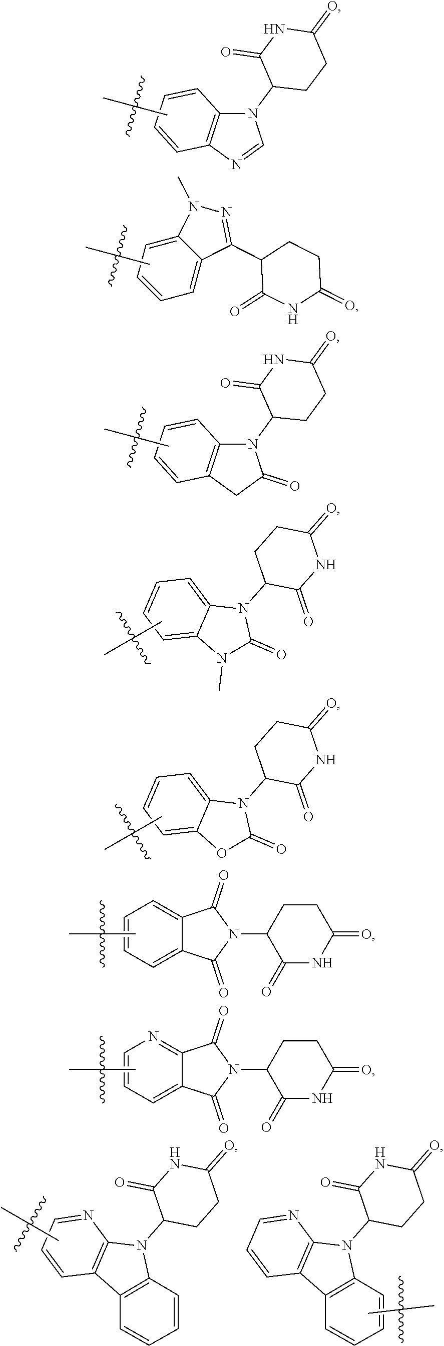 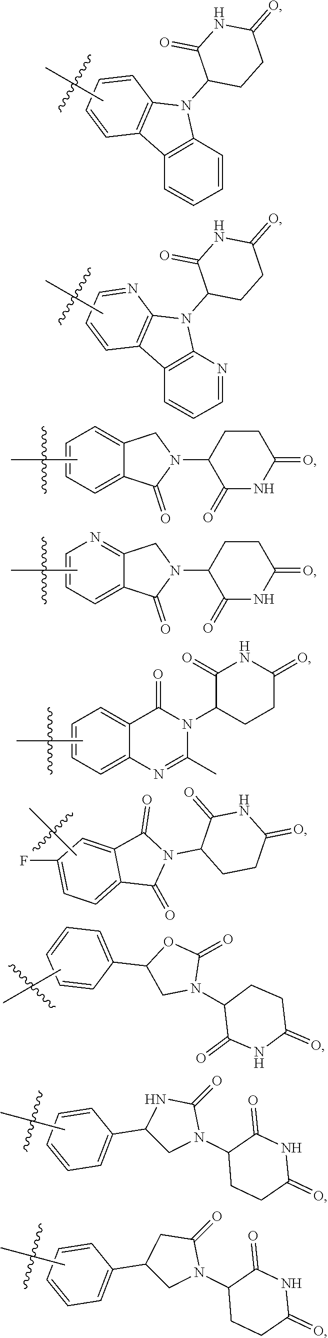 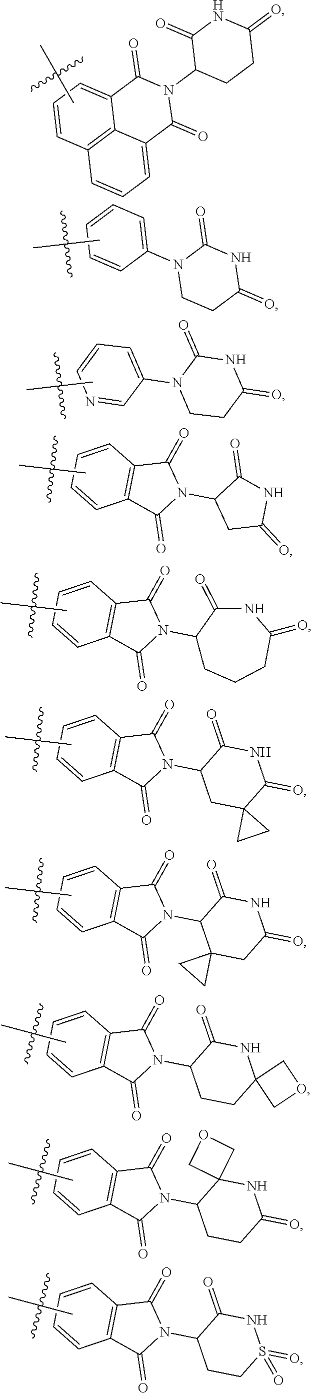 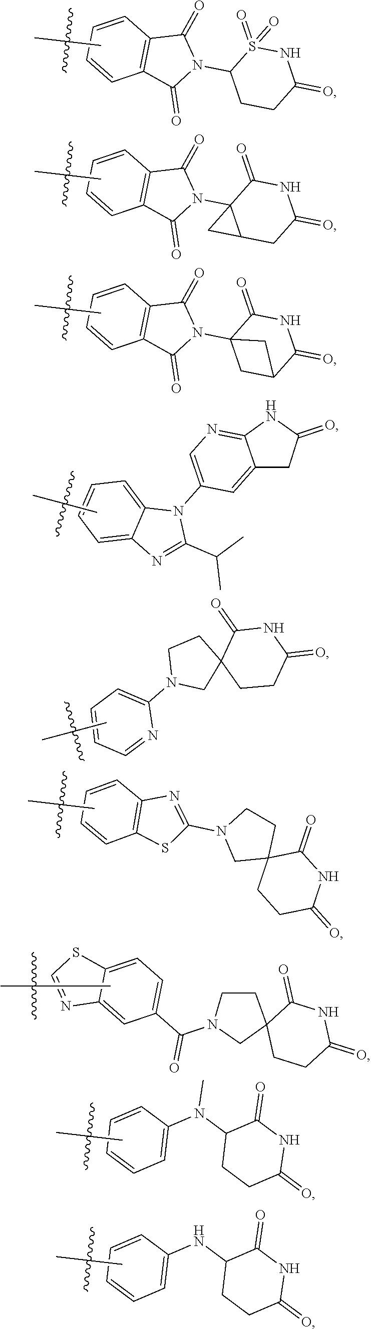 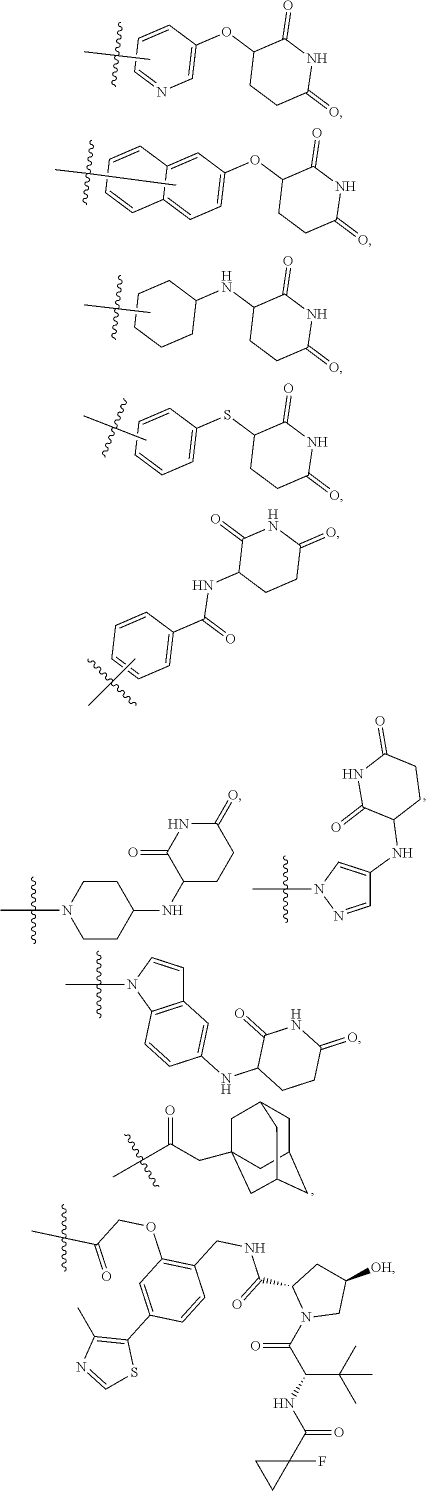 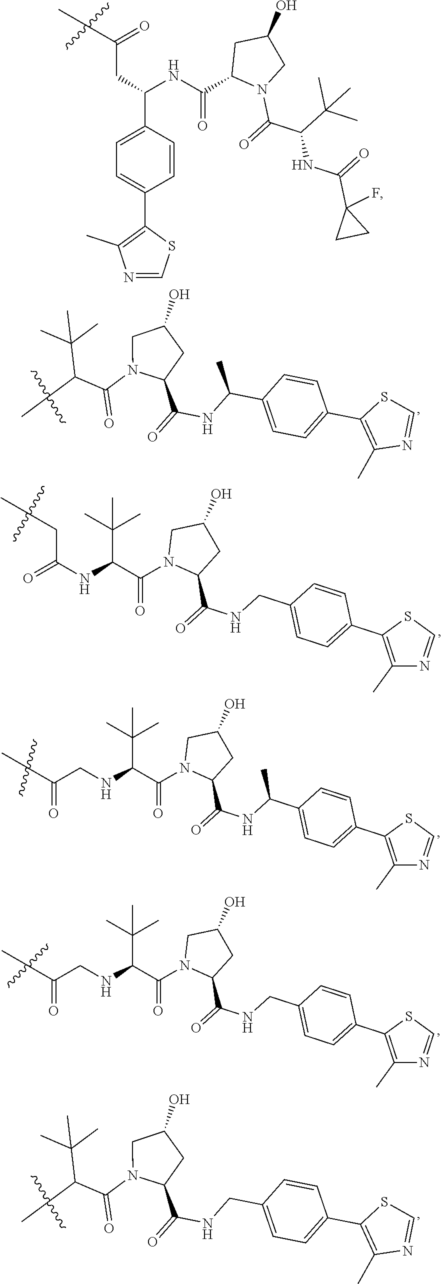 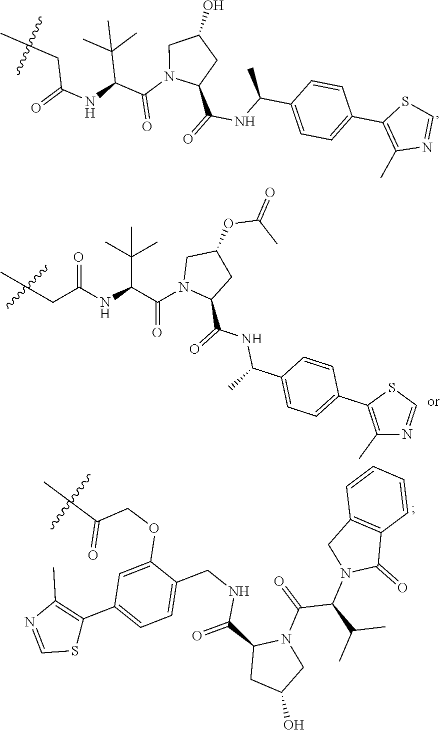
or K is selected from

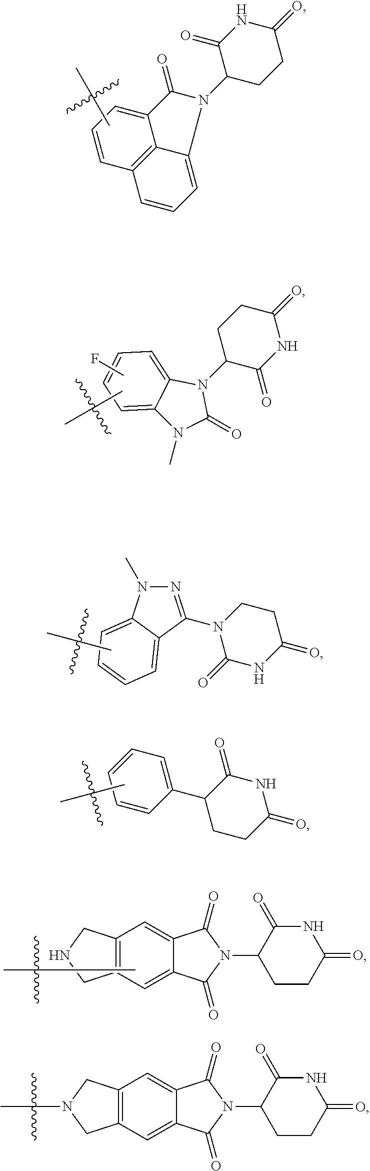

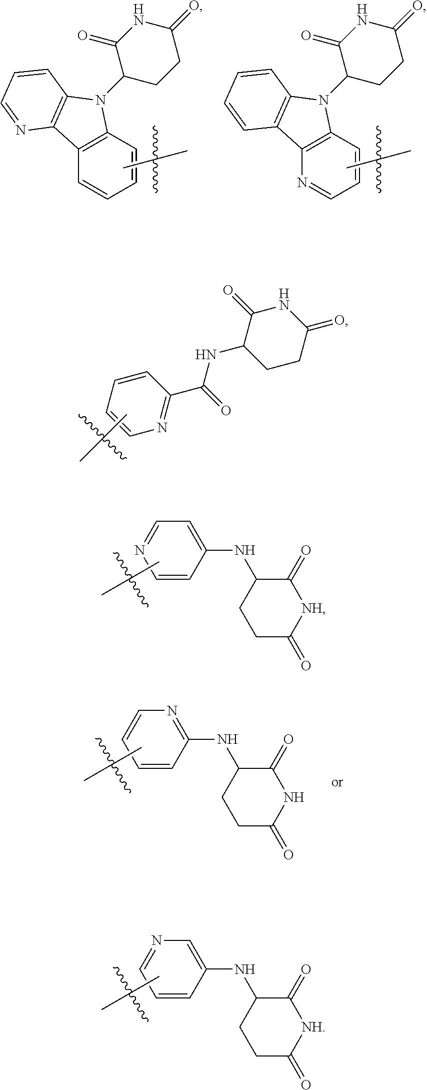

11. The compound or the stereoisomer, deuterated compound, solvate, prodrug, metabolite, pharmaceutically acceptable salt or co-crystal thereof according to claim 10, wherein
L is selected from -Cy1-, -Cy1-Ak1-, -Cy1-Ak1-Ak2-, -Cy1-Ak1-Ak2-Ak3-, -Cy1-Ak1-Ak2-Ak3-Ak4-, -Cy1-Cy2-, -Cy1-Ak1-Cy2-, -Cy1-Cy2-Ak2-, -Cy1-Ak1-Cy2-Ak2-, -Cy1-Ak1-Cy2-Ak2-Ak3-, -Cy1-Ak1-Cy2-Ak2-Ak3-Ak4-, -Cy1-Cy2-Ak2-Ak3-, -Cy1-Cy2-Ak2-Ak3-Ak4-, -Cy1-Ak1-Cy2-Ak2-Ak3-Ak4-, -Cy1-Ak1-Ak2-Cy3-, -Cy1-Ak1-Ak2-Cy3-Ak3-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Ak1-Cy2-Cy3-Ak3-, -Cy1-Cy2-Ak2-Cy3-Ak3-, -Cy1-Ak1-Cy2-Ak2-Cy3-, -Cy1-Ak1-Cy2-Ak2-Cy3-Ak3-, -Cy1-Cy2-Cy3-Ak3-Ak4-, -Cy1-Cy2-Cy3-Cy4-, -Cy1-Ak1-Cy2-Cy3-Cy4-, -Cy1-Cy2-Ak2-Cy3-Cy4-, -Cy1-Cy2-Cy3-Ak3-Cy4-, -Cy1-Cy2-Cy3-Cy4-Ak4-, -Cy1-Ak1-Cy2-Ak2-Cy3-Ak3-Cy4-, -Cy1-Ak1-Cy2-Ak2-Cy3-Cy4-, -Ak1-Ak2-Cy3-Cy4-, -Ak1-Cy2-Ak2-Cy3-, -Ak1-Cy2-Ak2-, -Ak1-Ak2-Ak3-Ak4-, -Ak1-Ak2-Ak3-, -Ak1-Ak2-, -Ak1-Ak2-Ak3-Ak4-Ak5-, -Cy1-Cy2-Cy3-Ak3-Ak4-Ak5-, -Cy1-Cy2-Ak2-Cy3-Ak3-Ak4-Ak5-, -Cy1-Ak1-Cy2-Ak2-Ak3-Ak4-Ak5-, -Cy1-Cy2-Cy3-Cy4-Ak4-Ak5-, -Cy1-Ak1-Ak2-Ak3-Ak4-Ak5-, -Ak1-Cy2-Ak2-Ak3-Ak4-Ak5-, -Ak1-Cy2-Ak2-Ak3-Ak4-, or -Ak1-Cy2-Ak2-Ak3-;
Ak1, Ak2, Ak3, Ak4, and Ak5 are each independently selected from —O—, —OCH₂—, —CH₂O—, —OCH₂CH₂—, —CH₂CH₂O—, —C≡C—, —CH₂—, —CH₂CH₂—, —CH₂CH₂CH₂—, —N(CH₃)—, —NH—, —CH₂N(CH₃)—, —CH₂NH—, —NHCH₂—, —CH₂CH₂N(CH₃)—, —CH₂CH₂NH—, —NHCH₂CH₂—, —C(═O)—, —C(═O)CH₂NH—, —CH₂C(═O)NH—, —C(═O)NH— or —NHC(═O)—;
preferably, L is selected from -Cy1-, -Cy1-Ak1-, -Cy1-Cy2-, -Cy1-Ak1-Cy2-, -Cy1-Cy2-Ak2-, -Cy1-Cy2-Cy3-, -Cy1-Ak1-Cy2-Cy3-, -Cy1-Cy2-Ak2-Cy3-, -Cy1-Cy2-Cy3-Ak3-, -Cy1-Cy2-Ak2-Cy3-Ak3- and -Cy1-Ak1-Cy2-Ak2-Cy3-;
Ak1, Ak2 and Ak3 are each independently selected from —C≡C—, —CH₂— and —CH₂—N(CH₃)—;
Cy1, Cy2 and Cy3 are each independently selected from 4- to 7-membered mono-heterocyclic ring, 4- to 10-membered fused heterocyclic ring, 5- to 12-membered spiro-heterocyclic ring, wherein the mono-heterocyclic ring, fused heterocyclic ring or spiro-heterocyclic ring is optionally further substituted with 0 to 4 substituents selected from H, F, Cl, Br, I, OH, COOH, CN, NH₂, ═O, C₁₋₄ alkyl, halogen-substituted C₁₋₄ alkyl, hydroxyl-substituted C₁₋₄ alkyl or C₁₋₄ alkoxy, and the mono-heterocyclic ring, fused heterocyclic ring or spiro-heterocyclic ring contains 1 to 4 heteroatoms selected from O, S, or N (preferably, N); preferably, Cy1, Cy2 and Cy3 are each independently selected from

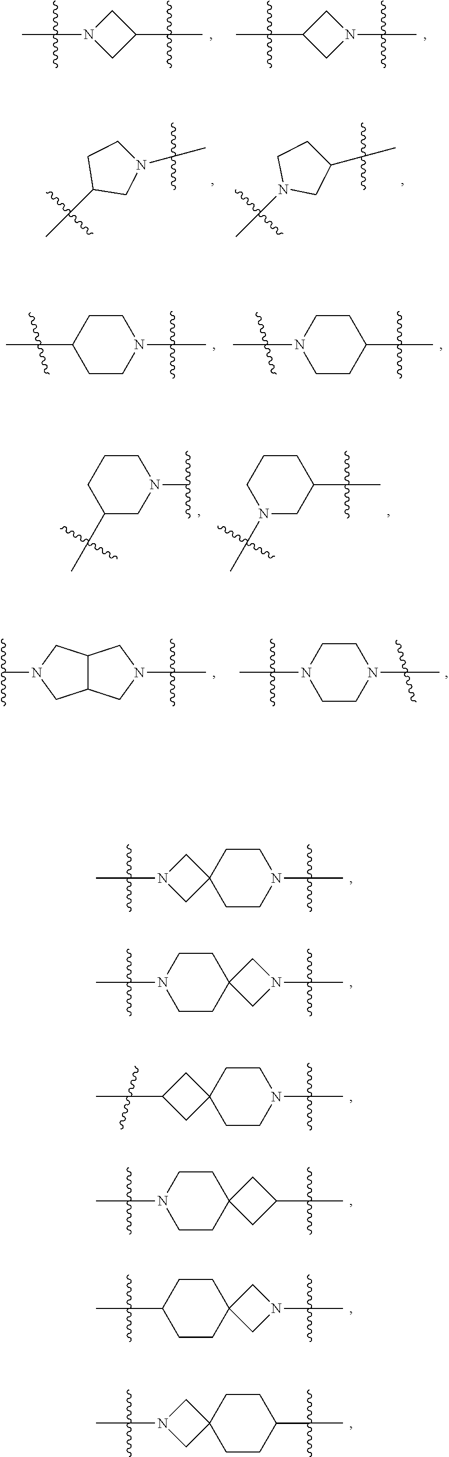

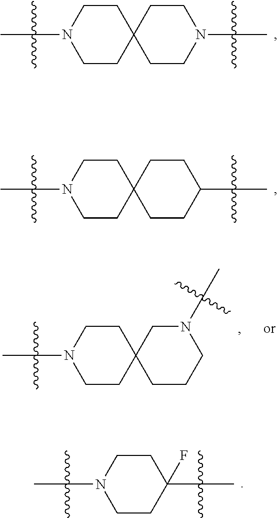 [SMILES: *N1CCC2(CC1)CCN(*)CC2 ; *C1CCC2(CC1)CCN(*)CC2 ; *N1CCC2(CCCN(*)C2)CC1 ; *N1CCC(*)(F)CC1]

12. The compound or the stereoisomer, deuterated compound, solvate, prodrug, metabolite, pharmaceutically acceptable salt or co-crystal thereof according to claim 11, wherein
L is selected from 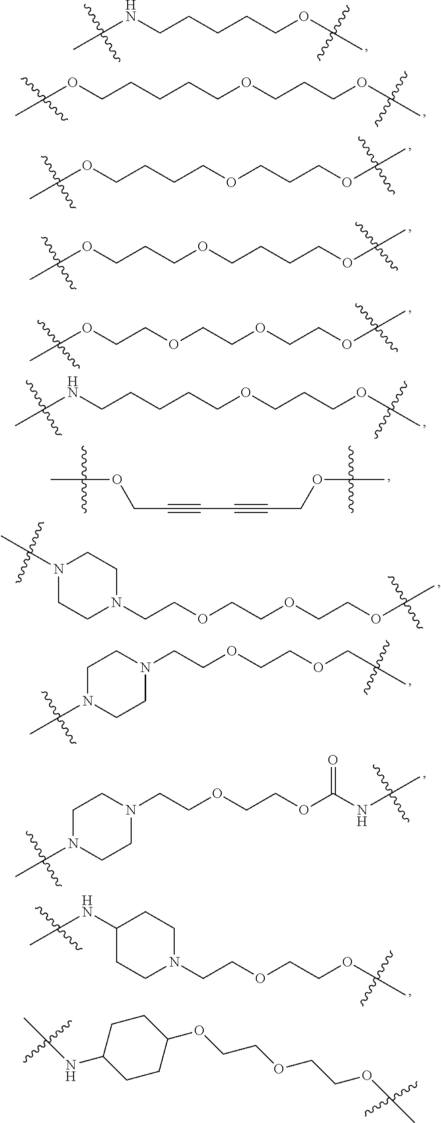 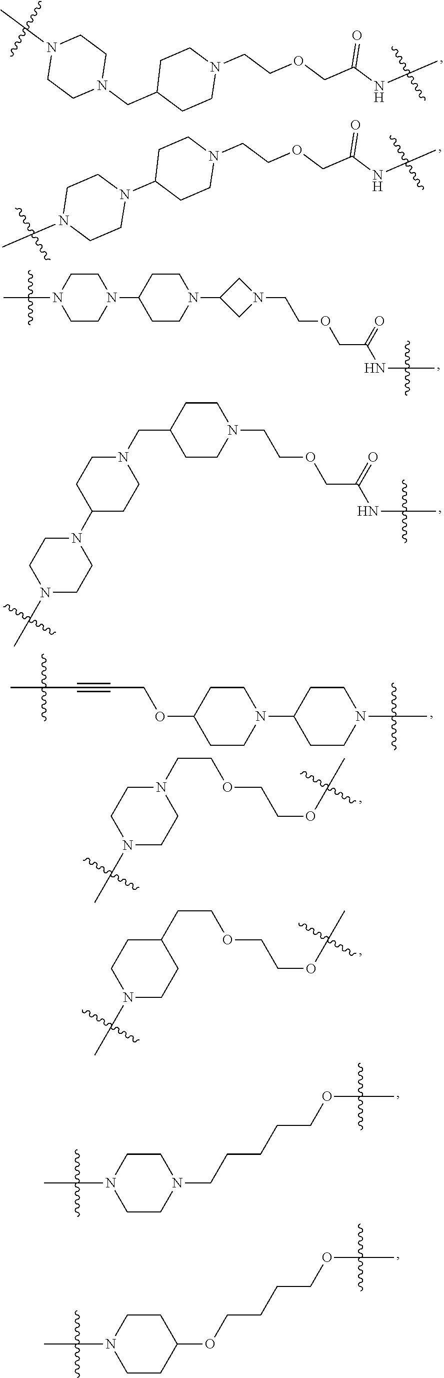 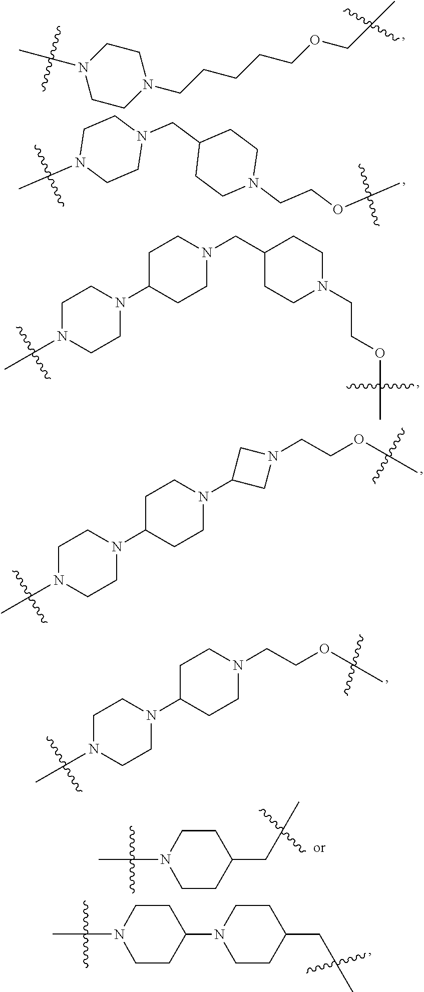
wherein the left side thereof is linked to B.

13. The compound or the stereoisomer, deuterated compound, solvate, prodrug, metabolite, pharmaceutically acceptable salt or co-crystal thereof according to claim 5, wherein
B is selected from 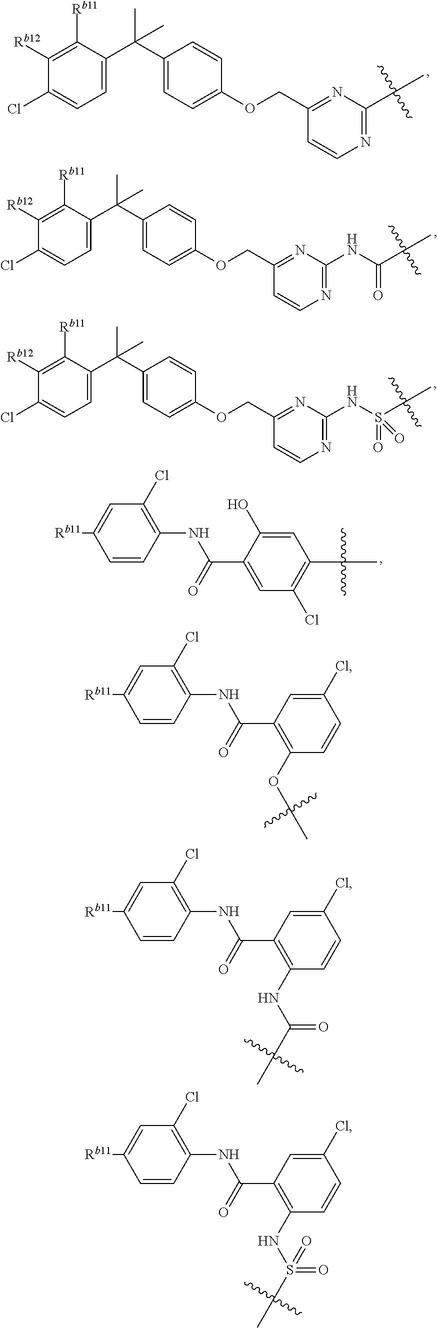 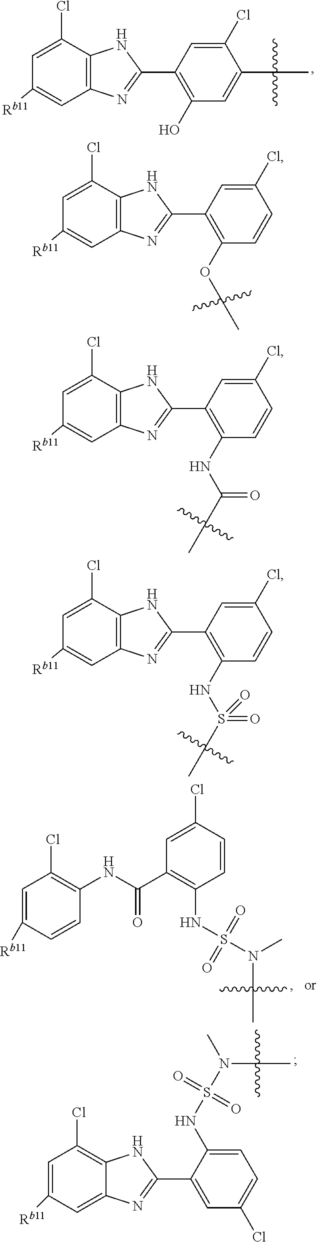
or B is selected from 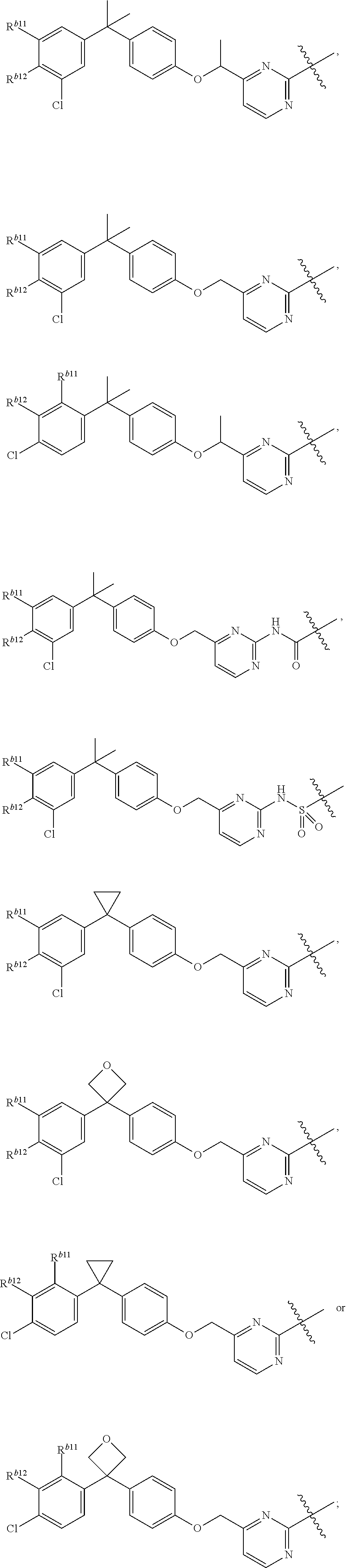
each R^{b11} is independently selected from F, Cl, Br, CF₃, CN, or NO₂;
each R^{b12} is independently selected from H, OCH₃, OCD₃, OCH₂CH₃, or OCH₂CH₂Cl;
K is selected from 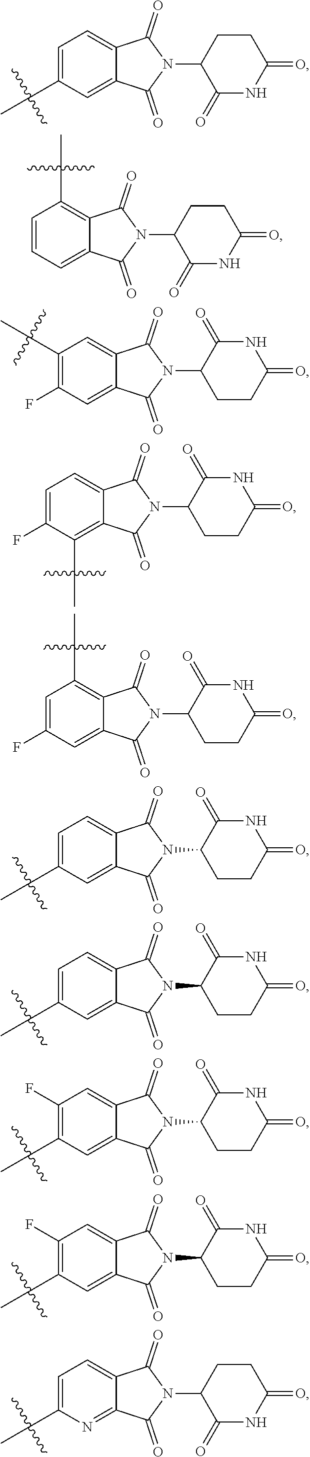 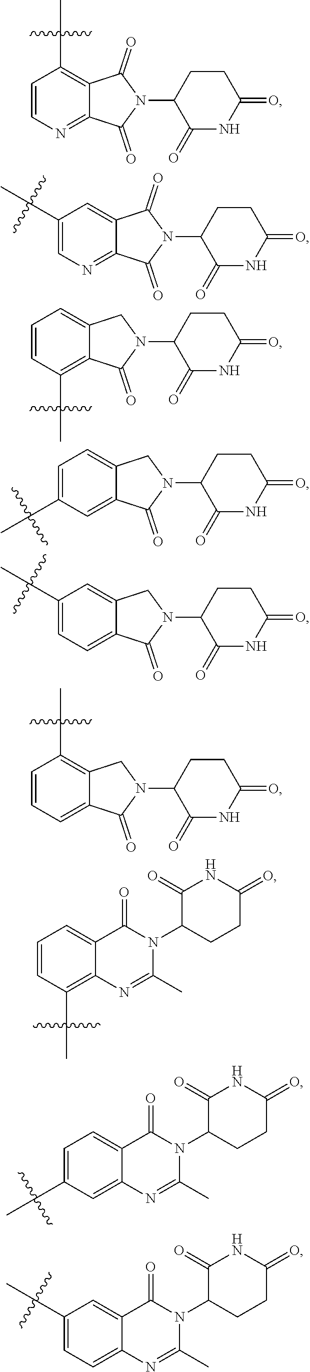 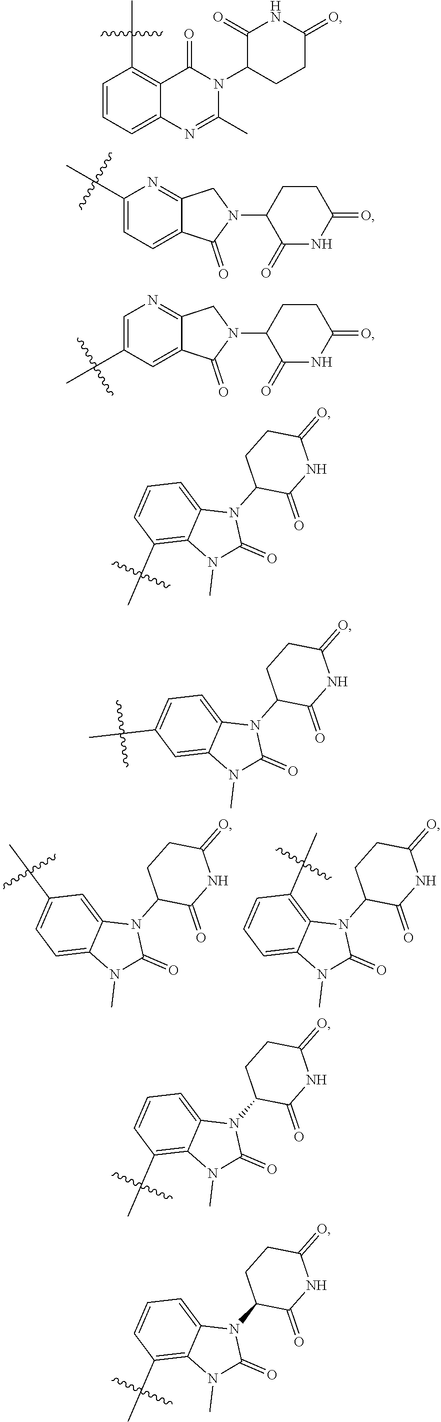 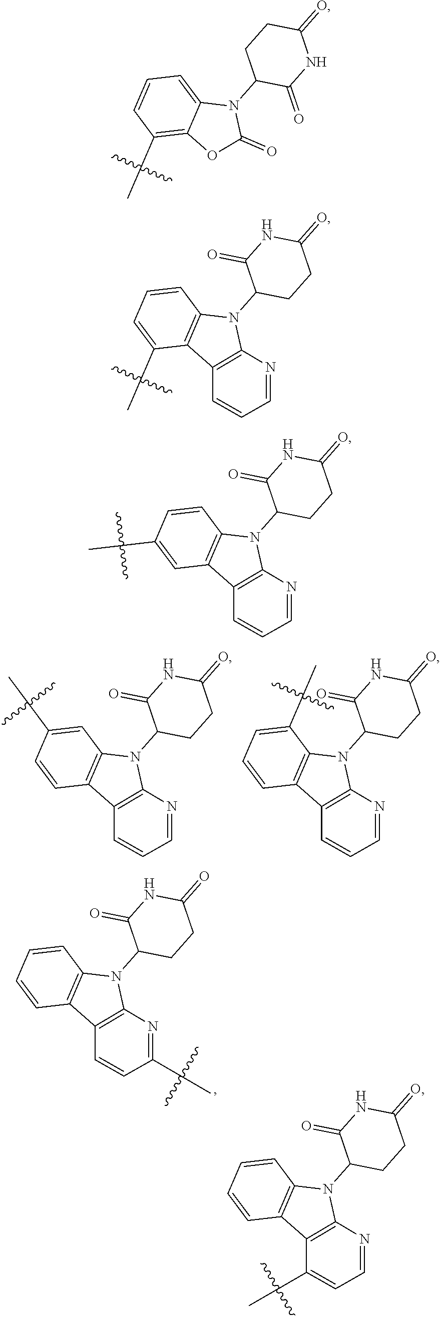 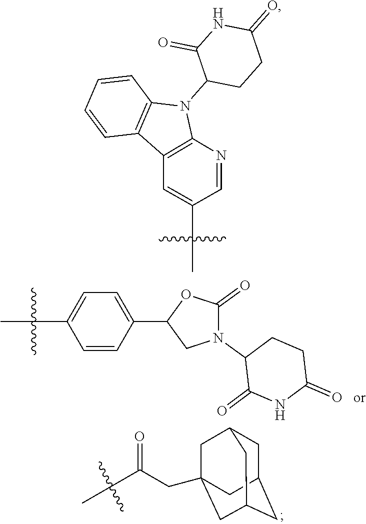
or K is selected from

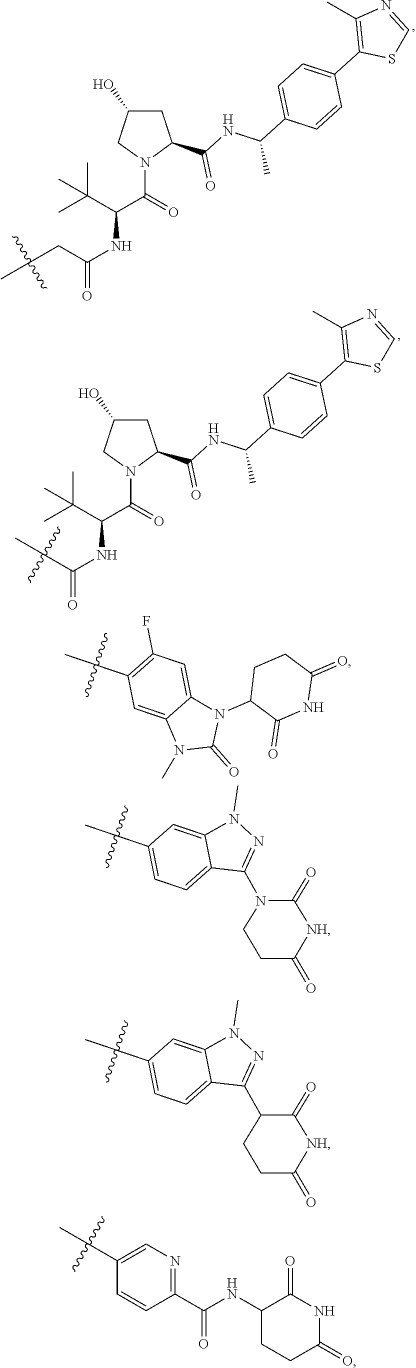

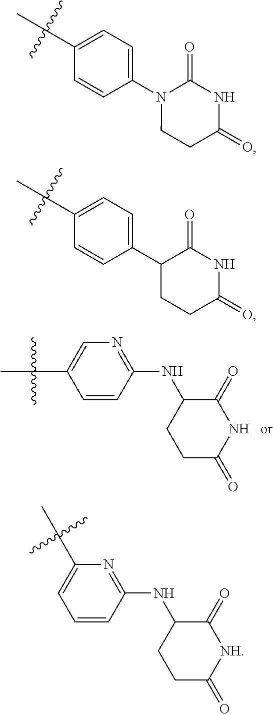

14. The compound or the stereoisomer, deuterated compound, solvate, prodrug, metabolite, pharmaceutically acceptable salt or co-crystal thereof according to claim 13, wherein
B is selected from 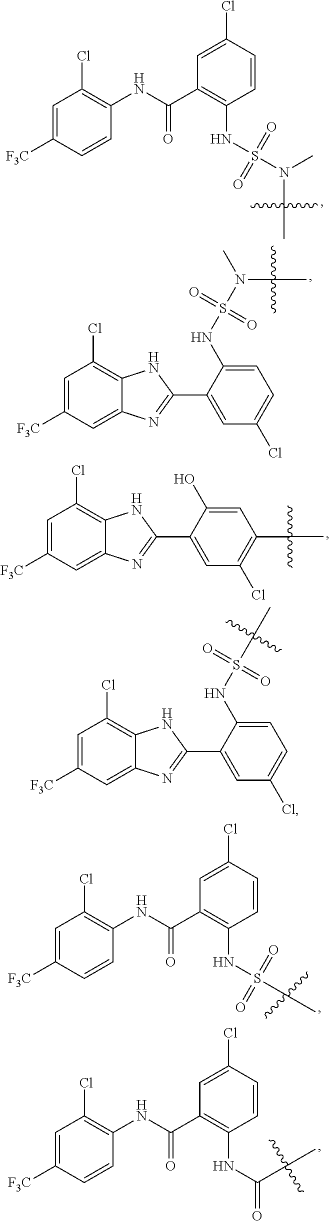 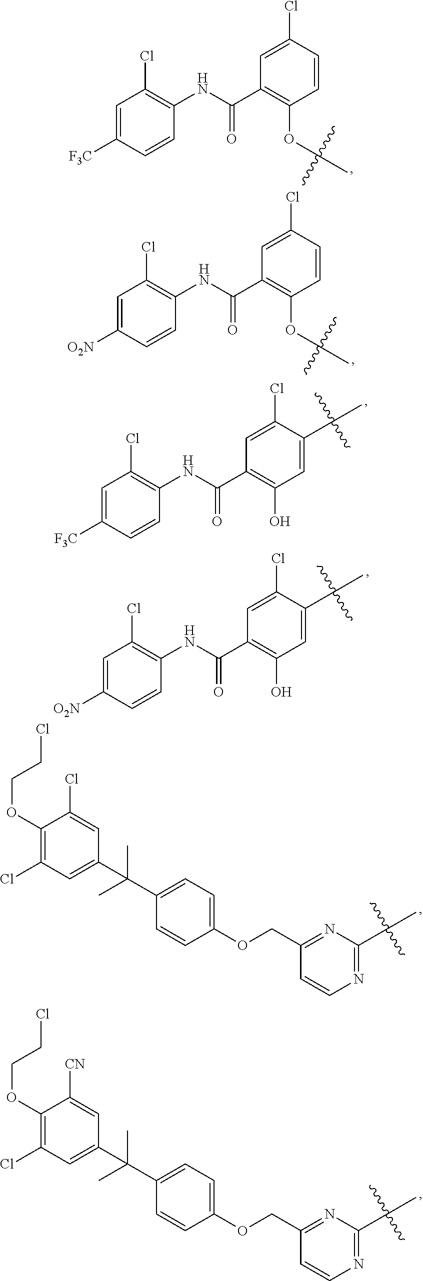 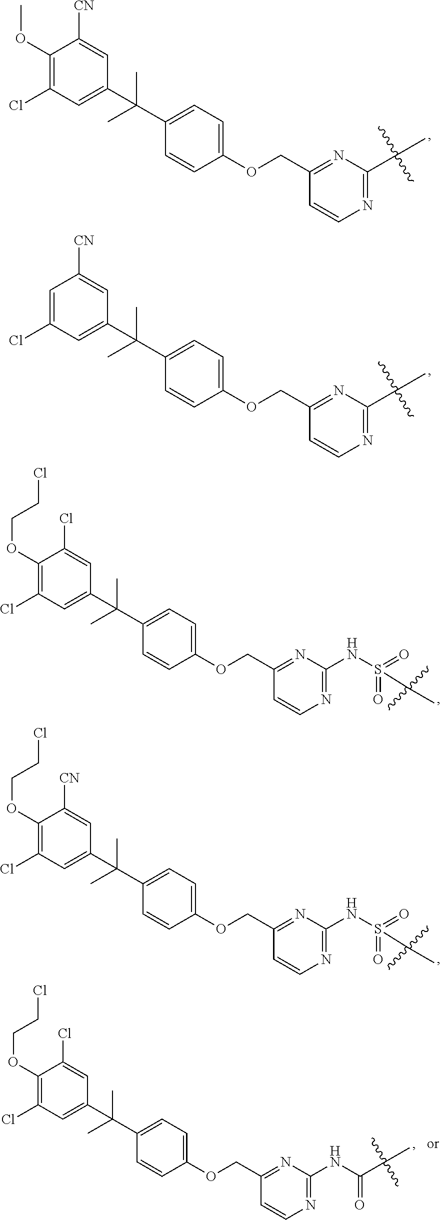 [SMILES: *c1nccc(COc2ccc(C(C)(C)c3cc(Cl)c(OC)c(C#N)c3)cc2)n1 ; *c1nccc(COc2ccc(C(C)(C)c3cc(Cl)cc(C#N)c3)cc2)n1 ; *S(=O)(=O)Nc1nccc(COc2ccc(C(C)(C)c3cc(Cl)c(OCCCl)c(Cl)c3)cc2)n1 ; *S(=O)(=O)Nc1nccc(COc2ccc(C(C)(C)c3cc(Cl)c(OCCCl)c(C#N)c3)cc2)n1 ; *C(=O)Nc1nccc(COc2ccc(C(C)(C)c3cc(Cl)c(OCCCl)c(Cl)c3)cc2)n1] 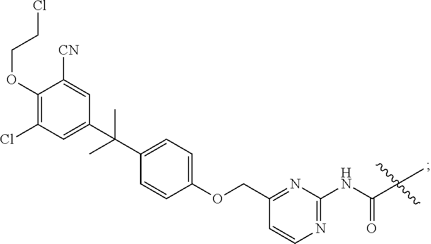 [SMILES: *C(=O)Nc1nccc(COc2ccc(C(C)(C)c3cc(Cl)c(OCCCl)c(C#N)c3)cc2)n1]
or B is selected from 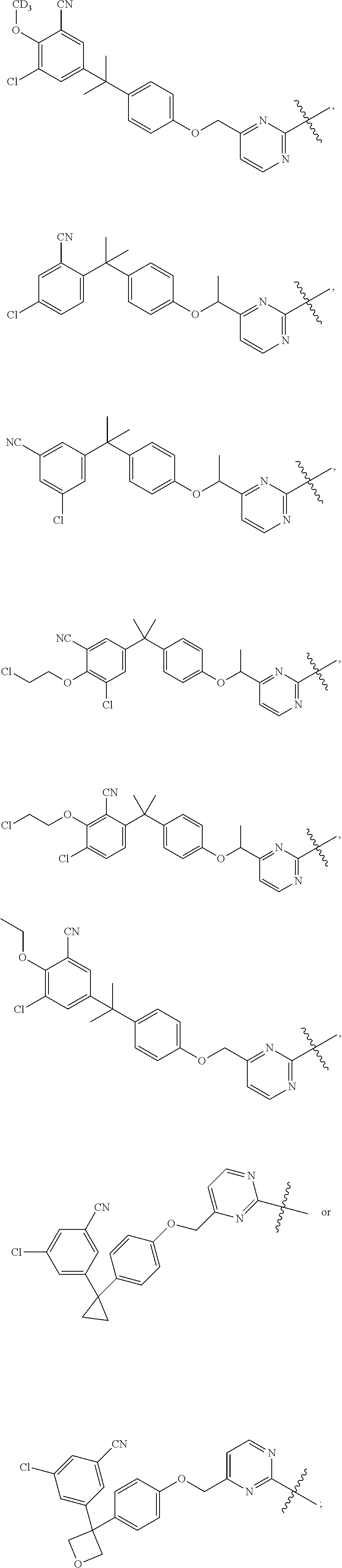
K is selected from 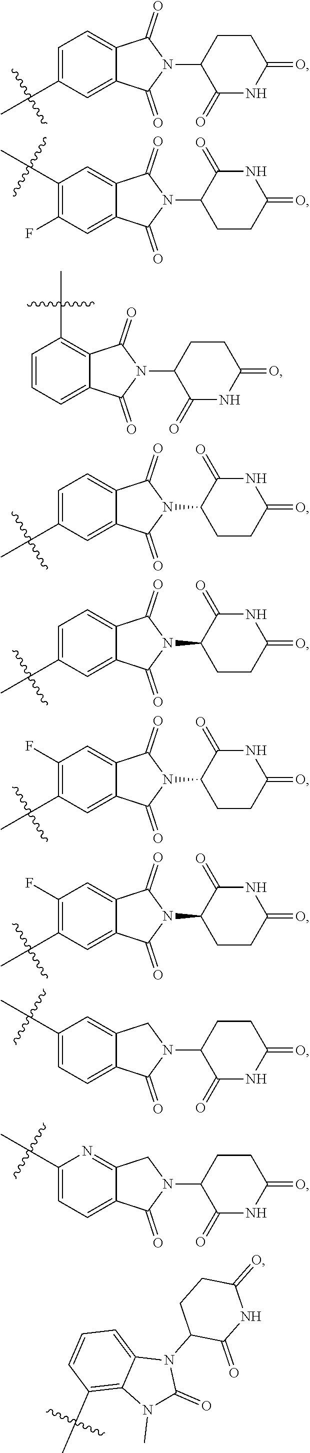 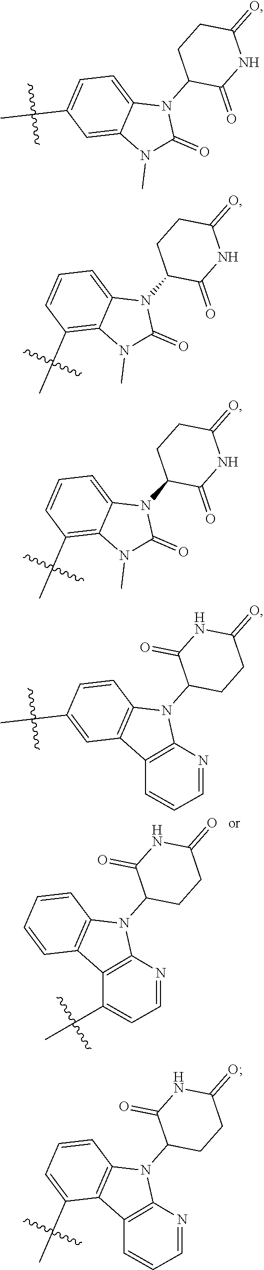
or K is selected from

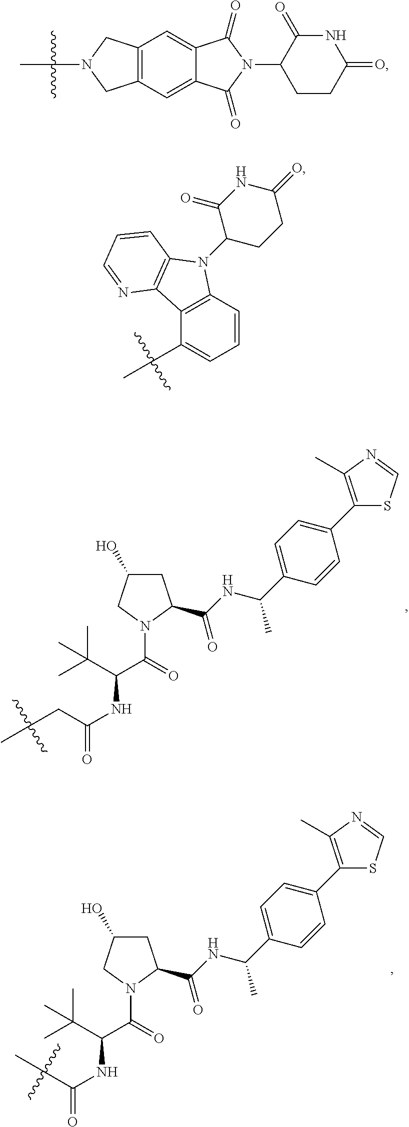 [SMILES: *N1Cc2cc3c(cc2C1)C(=O)N(C1CCC(=O)NC1=O)C3=O ; *c1cccc2c1c1ncccc1n2C1CCC(=O)NC1=O ; *CC(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

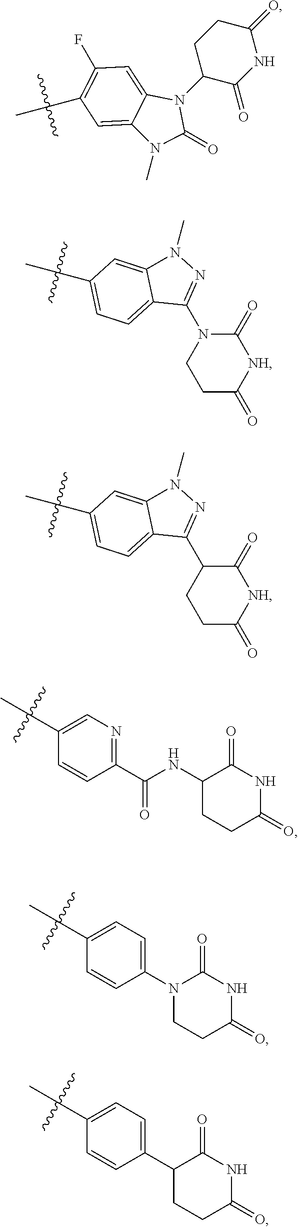 [SMILES: *c1cc2c(cc1F)n(C1CCC(=O)NC1=O)c(=O)n2C ; *c1ccc2c(N3CCC(=O)NC3=O)nn(C)c2c1 ; *c1ccc2c(C3CCC(=O)NC3=O)nn(C)c2c1 ; *c1ccc(C(=O)NC2CCC(=O)NC2=O)nc1 ; *c1ccc(N2CCC(=O)NC2=O)cc1 ; *c1ccc(C2CCC(=O)NC2=O)cc1]

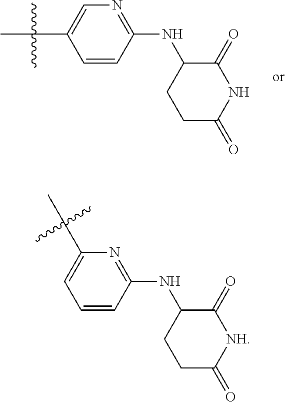 [SMILES: *c1ccc(NC2CCC(=O)NC2=O)nc1 ; *c1cccc(NC2CCC(=O)NC2=O)n1]

15. The compound or the stereoisomer, deuterated compound, solvate, prodrug, metabolite, pharmaceutically acceptable salt or co-crystal thereof according to claim 1, wherein the compound is selected from one of the compounds as shown in Table D.

16. A pharmaceutical composition, comprising the compound or the stereoisomer, deuterated compound, solvate, prodrug, metabolite, pharmaceutically acceptable salt or co-crystal thereof according to claim 1, and a pharmaceutically acceptable carrier.

17. (canceled)

18. (canceled)

19. (canceled)

20. A method for treating a disease related to abnormal activity or expression level of an AR or AR splice variant, comprising a step of administering to a subject in need thereof an effective amount of the compound or the stereoisomer, deuterated compound, solvate, prodrug, metabolite, pharmaceutically acceptable salt or co-crystal thereof according to claim 1.

21. The method according to claim 20, wherein the disease is an autoimmune disease, an inflammatory disease or cancer.

22. The method according to claim 21, wherein the cancer is prostate cancer.
